(19) United States
(12) Patent Application Publication
(10) Pub. No.: US 2020/0147235 A1
(43) Pub. Date: May 14, 2020

(54) ANTI-CD98 ANTIBODIES AND ANTIBODY DRUG CONJUGATES

(71) Applicant: AbbVie Inc., North Chicago, IL (US)

(72) Inventors: Lorenzo Benatuil, Northborough, MA (US); Milan Bruncko, Green Oaks, IL (US); Andrew S. Judd, Grayslake, IL (US); Yingchun Li, Buffalo Grove, IL (US); Andrew Mccluskey, Shrewsbury, MA (US); Andrew C. Phillips, Libertyville, IL (US); Darren C. Phillips, Glenview, IL (US); Jane Seagal, Newton, MA (US); Andrew J. Souers, Libertyville, IL (US)

(21) Appl. No.: 16/308,774

(22) PCT Filed: Jun. 8, 2017
(86) PCT No.: PCT/US2017/036645

§ 371 Date: Dec. 10, 2018

Related U.S. Application Data
(60) Provisional application No. 62/347,483, filed on Jun. 8, 2016.

(51) Int. Cl. A61K 47/68; A61K 31/337; C07K 16/28

(52) U.S. Cl. CPC A61K 47/6889; A61K 31/337; A61K 45/06; A61K 47/6803; A61K 47/6851; C07K 16/2896

(57) ABSTRACT

The invention relates to anti-CD98 antibodies and antibody drug conjugates (ADCs), including compositions and methods of using said antibodies and ADCs.

### RELATED APPLICATIONS

The present application is a 371 national phase application of PCT Application No. PCT/US2017/036645, which was filed on Jun. 8, 2017, and claims priority to U.S. Provisional Application No. 62/347,483, filed Jun. 8, 2016, the entire contents of which are hereby incorporated by reference herein.

### SEQUENCE LISTING

The instant application contains a Sequence Listing which has been submitted electronically in ASCII format and is hereby incorporated by reference in its entirety. Said ASCII copy, created on Jun. 1, 2017, is named 117813-10720_SL.txt and is 173,828 bytes in size.

### BACKGROUND OF THE INVENTION

CD98 (also referred to as CD98 heavy chain: 4F2 heavy chain; 4F2hc; SLC3A2) is an 80 kDa type II transmembrane glycoprotein chain which is known to be highly expressed in various types of cancer cells, CD98 forms a heterodimer with a protein of about 40 kDa having an amino acid transporter activity via a disulfide bond, and is expressed on the cell membrane. In particular, CD98 covalently links via a disulfide bond to one of several light chains (LAT1 (SLC7A5), SLC7A6, SLC7A7. SLC7A8, SLC7A10, or SLC7A11), which are L-type amino acid transporters. This interaction is required for the cell surface expression and amino acid transport function of the light chains, CD98 also associates with integrin f) subunits, thereby regulating integrin signaling that controls cell proliferation, survival, migration, and epithelial adhesion and polarity (Cai et al., J. Cell Sci. (2005) 1 18: 889-899; Haynes B. F, et al., J. Immunol., (1981), 126, 1409-1414; Lindsten T, et al., Mol. Cell. Biol., (1988), 8, 3820-3826; Teixeira S, et al., Eur. J. Biochem., (1991), 202, 819-826; L. A. Diaz Jr, et al., J Biol Regul Homeost Agents, (1998) 12, 25-32). The function of CD98 in regulating both amino acid transport and integrin signaling can contribute to the rapid proliferation and clonal expansion of lymphocytes and tumor cells (Cantor, et al. (2012) J. Cell Sci. 125:1373-82).

CD98 is overexpressed on the cell surface of almost all tumor cells, regardless of tissue origin, and increased expression of L-type amino acid transporter 1 (LAT 1; also known as SLC7A5) occurs in many types of human cancers, including breast, colon, oral, ovarian, esophageal, glioma and leukemia (Cantor (2012) J Cell Sci 2012; 125:1373-82). LAT1 forms a complex with CD98 and transports neutral amino acids having large side chains, such as leucine, valine, phenylalanine, tyrosine, tryptophan, methionine, histidine and the like in a sodium ion-independent manner. In addition, LAT1 is poorly, or not expressed in most normal tissues except for the brain, placenta, bone marrow and testis, but its expression increases together with CD98 in tissues of several human malignant tumors (Yanagida et al., Biochem. Biophys. Acta (2001), 1514, 291-302).

CD98 has been associated with cancer, see, for example, Estrach et al. (2014) Cancer Res 74(23): 6878) and Cantor and Ginsberg (2012) J Cell Sci 125(6):1373. The expression of CD98 is significantly higher in metastatic sites of human cancers than in the primary sites, suggesting that overexpression of LAT1/CD98 may be important for progression and metastasis of human cancers (Hayes, et al. International Journal of Cancer (2015) 137, 710-720). For example, LAT1/CD98 overexpression appears to be required for tumor metastasis in patients with colon cancer (Kaira et al., Cancer Sci. (2008) 99: 2380-2386). In addition, positive expression of CD98 was an independent factor for predicting a poor prognosis in resected non-small-cell lung cancer (Kaira et al., Ann. Surgical Oncol. (2009) 16(12):3473-81) and the overexpression of LAT1 and CD98 was found to be a pathological factor for prediction of prognosis in patients with resectable stage I pulmonary adenocarcinoma (Kaira et al., Lung Cancer (2009) 66:1.120-126.

Antibody drug conjugates (ADC) represent relatively a class of therapeutics comprising an antibody conjugated to a cytotoxic drug via a chemical linker. The therapeutic concept of ADCs is to combine binding capabilities of an antibody with a drug, where the antibody is used to deliver the drug to a tumor cell by means of binding to a target surface antigen.

Accordingly, there remains a need in the art for anti-CD98 antibodies and ADCs that can be used for therapeutic purposes in the treatment of cancer.

### SUMMARY OF THE INVENTION

In certain aspects, the present invention provides for anti-CD98 antibodies and antibody drug conjugates (ADCs) that specifically bind to CD98.

In certain embodiments of the invention, the antibodies, or antigen binding portions thereof, bind to CD98 (SEQ ID NO: 124) or the extracellular domain of CD98 (SEQ ID NO: 125), with a K_{d} of between about 1×10⁻⁶ M and about 1×10⁻¹¹ M, as determined by surface plasmon resonance.

In yet other embodiments of the invention, the anti-CD98 antibody drug conjugates (ADCs), e.g., an anti-CD98 antibody conjugated to a Bcl-xL inhibitor, inhibits tumor growth in an in vivo human non-small-cell lung carcinoma (NSCLC) xenograft assay.

In some embodiments, the antibody, or antigen binding portion thereof, that binds to human CD98, comprises a heavy chain variable region comprising a CDR3 having the amino acid sequence of SEQ ID NO: 17 and a light chain variable region comprising a CDR3 having the amino acid sequence of SEQ ID NO: 19. In other embodiments, the anti-CD98 antibody, or antigen binding portion thereof, comprises a heavy chain variable region comprising a CDR2 having the amino acid sequence of SEQ ID NO: 87 and a light chain variable region comprising a CDR2 having the amino acid sequence of SEQ ID NO: 7. In other embodiments, the anti-CD98 antibody, or antigen binding portion thereof, comprises a heavy chain variable region comprising a CDR1 having the amino acid sequence of SEQ ID NO: 16 and a light chain variable region comprising a CDR1 having the amino acid sequence of either SEQ ID NO: 13.

In some embodiments, the anti-CD98 antibody, or antigen binding portion thereof, that binds to human CD98, comprises a heavy chain variable region comprising a CDR3 having the amino acid sequence of SEQ ID NO: 17 and a light chain variable region comprising a CDR3 having the amino acid sequence of SEQ ID NO: 19. In other embodiments, the anti-CD98 antibody, or antigen binding portion thereof, comprises a heavy chain variable region comprising a CDR2 having the amino acid sequence of SEQ ID NO: 90, and a light chain variable region comprising a CDR2 having the amino acid sequence of SEQ ID NO: 7. In other embodiments, the anti-CD98 antibody, or antigen binding portion thereof, comprises a heavy chain variable region comprising a CDR1 having the amino acid sequence of SEQ ID NO: 16 and a light chain variable region comprising a CDR1 having the amino acid sequence of either SEQ ID NO: 13.

In some embodiments, the antibody, or antigen binding portion thereof, that binds to human CD98, comprises a heavy chain variable region comprising a CDR3 having the amino acid sequence of SEQ ID NO: 97 and a light chain variable region comprising a CDR3 having the amino acid sequence of SEQ ID NO: 95. In other embodiments, the anti-CD98 antibody, or antigen binding portion thereof, comprises a heavy chain variable region comprising a CDR2 having the amino acid sequence of SEQ ID NO: 92, and a light chain variable region comprising a CDR2 having the amino acid sequence of SEQ ID NO: 45. In other embodiments, the anti-CD98 antibody, or antigen binding portion thereof, comprises a heavy chain variable region comprising a CDR1 having the amino acid sequence of SEQ ID NO: 79 and a light chain variable region comprising a CDR1 having the amino acid sequence of either SEQ ID NO: 83.

In some embodiments, the antibody, or antigen binding portion thereof, that binds to human CD98, comprises a heavy chain variable region comprising a CDR3 having the amino acid sequence of SEQ ID NO: 97 and a light chain variable region comprising a CDR3 having the amino acid sequence of SEQ ID NO: 102. In other embodiments, the anti-CD98 antibody, or antigen binding portion thereof, comprises a heavy chain variable region comprising a CDR2 having the amino acid sequence of SEQ ID NO: 104, and a light chain variable region comprising a CDR2 having the amino acid sequence of SEQ ID NO: 45. In other embodiments, the anti-CD98 antibody, or antigen binding portion thereof, comprises a heavy chain variable region comprising a CDR1 having the amino acid sequence of SEQ ID NO: 79 and a light chain variable region comprising a CDR1 having the amino acid sequence of either SEQ ID NO: 83.

In some embodiments, the anti-CD98 antibody, or antigen binding portion thereof, comprises a heavy chain CDR3 domain comprising the amino acid sequence set forth in SEQ ID NO: 17, a heavy chain CDR2 domain comprising the amino acid sequence set forth in SEQ ID NO: 87, and a heavy chain CDR1 domain comprising the amino acid sequence set forth in SEQ ID NO: 16; and a light chain CDR3 domain comprising the amino acid sequence set forth in SEQ ID NO: 19, a light chain CDR2 domain comprising the amino acid sequence set forth in SEQ ID NO: 7, and a light chain CDR1 domain comprising the amino acid sequence set forth in SEQ ID NO: 13. In yet another embodiment, the anti-CD98 antibody, or antigen binding portion thereof, comprises a heavy-chain variable region comprising the amino acid sequence set forth in SEQ ID NO: 108, and a light chain variable region comprising the amino acid sequence set forth in SEQ ID NO: 107.

In some embodiments, an anti-CD98 antibody, or antigen-binding portion thereof, comprises a heavy chain comprising an amino acid sequence set forth in SEQ ID NO: 108, or a sequence having at least 90%, 95%, 96%, 97%, 98%, or 99% identity to SEQ ID NO: 108, and/or a light chain comprising an amino acid sequence set forth in SEQ ID NO: 107, or a sequence having at least 90%, 95%, 96%, 97%, 98%, or 99% identity to SEQ ID NO: 107.

In some embodiments, the anti-CD98 antibody, or antigen binding portion thereof, comprises a heavy chain CDR3 domain comprising the amino acid sequence set forth in SEQ ID NO: 17, a heavy chain CDR2 domain comprising the amino acid sequence set forth in SEQ ID NO: 90, and a heavy chain CDR1 domain comprising the amino acid sequence set forth in SEQ ID NO: 16; and a light chain CDR3 domain comprising the amino acid sequence set forth in SEQ ID NO: 19, a light chain CDR2 domain comprising the amino acid sequence set forth in SEQ ID NO: 7, and a light chain CDR1 domain comprising the amino acid sequence set forth in SEQ ID NO: 13. In yet another embodiment, the anti-CD98 antibody, or antigen binding portion thereof, comprises a heavy chain variable region comprising the amino acid sequence set forth in SEQ ID NO: 110, and a light chain variable region comprising the amino acid sequence set forth in SEQ ID NO: 107.

In some embodiments, an anti-CD98 antibody, or antigen-binding portion thereof, comprises an amino acid sequence set forth in SEQ ID NO: 110, or a sequence having at least 90%, 95%, 96%, 97%, 98%, or 99% identity to SEQ ID NO: 110, and/or a light chain comprising an amino acid sequence set forth in SEQ ID NO: 107, or a sequence having at least 90%, 95%, 96%, 97%, 98%, or 99% identity- to SEQ ID NO: 107.

In some embodiments, the anti-CD98 antibody, or antigen binding portion thereof, comprises a heavy chain CDR3 domain comprising the amino acid sequence set forth in SEQ ID NO: 97, a heavy chain CDR2 domain comprising the amino acid sequence set forth in SEQ ID NO: 92, and a heavy chain CDR1 domain comprising the amino acid sequence set forth in SEQ ID NO: 79; and a light chain CDR3 domain comprising the amino acid sequence set forth in SEQ ID NO: 95, a light chain CDR2 domain comprising the amino acid sequence set forth in SEQ ID NO: 45, and a light chain CDR1 domain comprising the amino acid sequence set forth in SEQ ID NO: 83. In yet another embodiment the anti-CD98 antibody, or antigen binding portion thereof, comprises a heavy chain variable region comprising the amino acid sequence set forth in SEQ ID NO: 115, and a light chain variable region comprising the amino acid sequence set forth in SEQ ID NO: 112.

In some embodiments, an anti-CD98 antibody, or antigen-binding portion thereof, comprises an amino acid sequence set forth in SEQ ID NO: 115, or a sequence having at least 90%, 95%, 96%, 97%, 98%, or 99% identity to SEQ ID NO: 115, and/or a light chain comprising an amino acid sequence set forth in SEQ ID NO: 112, or a sequence having at least 90% 95%, 96% 97%, 98% or 99% identity- to SEQ ID NO: 112.

In some embodiments, the anti-CD98 antibody, or antigen binding portion thereof, comprises a heavy chain CDR3 domain comprising the amino acid sequence set forth in SEQ ID NO: 97, a heavy chain CDR2 domain comprising the amino acid sequence set forth in SEQ ID NO: 104, and a heavy chain CDR1 domain comprising the amino acid sequence set forth in SEQ ID NO: 79; and a light chain CDR3 domain comprising the amino acid sequence set forth in SEQ ID NO: 102, a light chain CDR2 domain comprising the amino acid sequence set forth in SEQ ID NO: 45, and a light chain CDR1 domain comprising the amino acid sequence set forth in SEQ ID NO: 83. In yet another embodiment the anti-CD98 antibody, or antigen binding portion thereof, comprises a heavy chain variable region comprising the amino acid sequence set forth in SEQ ID NO: 118, and a light chain variable region comprising the amino acid sequence set forth in SEQ ID NO: 117.

In some embodiments, an anti-CD98 antibody, or antigen-binding portion thereof, comprises an amino acid sequence set forth in SEQ ID NO: 118, or a sequence having at least 90%, 95%, 96%, 97%, 98%, or 99% identity- to SEQ ID NO: 118, and/or a light chain comprising an amino acid sequence set forth in SEQ ID NO: 117, or a sequence having at least 90% 95%, 96% 97%, 98% or 99% identity to SEQ ID NO: 117.

In some embodiments, the antibody—that binds to human CD98, comprises a heavy chain variable region comprising a CDR3 having the amino acid sequence of SEQ ID NO: 17 and a light chain variable region comprising a CDR3 having the amino acid sequence of SEQ ID NO: 19. In other embodiments, the antibody comprises a heavy-chain variable region comprising a CDR2 having the amino acid sequence of SEQ ID NO: 87 and a light chain variable region comprising a CDR2 having the amino acid sequence of SEQ ID NO: 7. In other embodiments, the anti-CD98 antibody comprises a heavy chain variable region comprising a CDR1 having the amino acid sequence of SEQ ID NO: 16 and a light chain variable region comprising a CDR1 having the amino acid sequence of either SEQ ID NO: 13.

In some embodiments, the antibody that binds to human CD98, comprises a heavy chain variable region comprising a CDR3 having the amino acid sequence of SEQ ID NO: 17 and a light chain variable region comprising a CDR3 having the amino acid sequence of SEQ ID NO: 19. In other embodiments, the anti-CD98 antibody comprises a heavy chain variable region comprising a CDR2 having the amino acid sequence of SEQ ID NO: 90, and a light chain variable region comprising a CDR2 having the amino acid sequence of SEQ ID NO: 7. In other embodiments, the anti-CD98 antibody comprises a heavy chain variable region comprising a CDR1 having the amino acid sequence of SEQ ID NO: 16 and a light chain variable region comprising a CDR1 having the amino acid sequence of either SEQ ID NO: 13.

In some embodiments, the antibody that binds to human CD98, comprises a heavy chain variable region comprising a CDR3 having the amino acid sequence of SEQ ID NO: 97 and a light chain variable region comprising a CDR3 having the amino acid sequence of SEQ ID NO: 95. In other embodiments, the anti-CD98 antibody comprises a heavy chain variable region comprising a CDR2 having the amino acid sequence of SEQ ID NO: 92, and a light chain variable region comprising a CDR2 having the amino acid sequence of SEQ ID NO: 45. In other embodiments, the anti-CD98 antibody comprises a heavy chain variable region comprising a CDR1 having the amino acid sequence of SEQ ID NO: 79 and a light chain variable region comprising a CDR1 having the amino acid sequence of either SEQ ID NO: 83.

In some embodiments, the antibody that binds to human CD98, comprises a heavy chain variable region comprising a CDR3 having the amino acid sequence of SEQ ID NO: 97 and a light chain variable region comprising a CDR3 having the amino acid sequence of SEQ ID NO: 102. In other embodiments, the anti-CD98 antibody comprises a heavy chain variable region comprising a CDR2 having the amino acid sequence of SEQ ID NO: 104, and a light chain variable region comprising a CDR2 having the amino acid sequence of SEQ ID NO: 45. In other embodiments, the anti-CD98 antibody comprises a heavy chain variable region comprising a CDR1 having the amino acid sequence of SEQ ID NO: 79 and a light chain variable region comprising a CDR1 having the amino acid sequence of either SEQ ID NO: 83.

In some embodiments, the anti-CD98 antibody comprises a heavy chain CDR3 domain comprising the amino acid sequence set forth in SEQ ID NO: 17, a heavy chain CDR2 domain comprising the amino acid sequence set forth in SEQ ID NO: 87, and a heavy chain CDR1 domain comprising the amino acid sequence set forth in SEQ ID NO: 16; and a light chain CDR3 domain comprising the amino acid sequence set forth in SEQ ID NO: 19, a light chain CDR2 domain comprising the amino acid sequence set forth in SEQ ID NO: 7, and a light chain CDR1 domain comprising the amino acid sequence set forth in SEQ ID NO: 13. In yet another embodiment, the anti-CD98 antibody comprises a heavy chain variable region comprising the amino acid sequence set forth in SEQ ID NO: 108, and a light chain variable region comprising the amino acid sequence set forth in SEQ ID NO: 107.

In some embodiments, an anti-CD98 antibody comprises a heavy chain comprising an amino acid sequence set forth in SEQ ID NO: 108, or a sequence having at least 90%, 95%, 96%, 97%, 98%, or 99% identity to SEQ ID NO: 108, and/or a light chain comprising an amino acid sequence set forth in SEQ ID NO: 107, or a sequence having at least 90%, 95%, 96%, 97%, 98%, or 99% identity to SEQ ID NO: 107.

In some embodiments, the anti-CD98 antibody comprises a heavy chain CDR3 domain comprising the amino acid sequence set forth in SEQ ID NO: 17, a heavy chain CDR2 domain comprising the amino acid sequence set forth in SEQ ID NO: 90, and a heavy chain CDR1 domain comprising the amino acid sequence set forth in SEQ ID NO: 16; and a light chain CDR3 domain comprising the amino acid sequence set forth in SEQ ID NO: 19, a light chain CDR2 domain comprising the amino acid sequence set forth in SEQ ID NO: 7, and a light chain CDR1 domain comprising the amino acid sequence set forth in SEQ ID NO: 13. In yet another embodiment, the anti-CD98 antibody comprises a heavy chain variable region comprising the amino acid sequence set forth in SEQ ID NO: 110, and a light chain variable region comprising the amino acid sequence set forth in SEQ ID NO: 107.

In some embodiments, ananti-CD98 antibody comprises an amino acid sequence set forth in SEQ ID NO: 110, or a sequence having at least 90%, 95%, 96%, 97%, 98%, or 99% identity to SEQ ID NO: 110, and/or a light chain comprising an amino acid sequence set forth in SEQ ID NO: 107, or a sequence having at least 90%, 95%, %%, 97%, 98%, or 99% identity to SEQ ID NO: 107.

In some embodiments, the anti-CD98 antibody comprises a heavy chain CDR3 domain comprising the amino acid sequence set forth in SEQ ID NO: 97, a heavy chain CDR2 domain comprising the amino acid sequence set forth in SEQ ID NO: 92, and a heavy chain CDR1 domain comprising the amino acid sequence set forth in SEQ ID NO: 79; and a light chain CDR3 domain comprising the amino acid sequence set forth in SEQ ID NO: 95, a light chain CDR2 domain comprising the amino acid sequence set forth in SEQ ID NO: 45, and a light chain CDR1 domain comprising the amino acid sequence set forth in SEQ ID NO: 83. In yet another embodiment, the anti-CD98 antibody comprises a heavy chain variable region comprising the amino acid sequence set forth in SEQ ID NO: 115, and a light chain variable region comprising the amino acid sequence set forth in SEQ ID NO: 112.

In some embodiments, an anti-CD98 antibody comprises an amino acid sequence set forth in SEQ ID NO: 115, or a sequence having at least 90%, 95%, 96%, 97%, 98%, or 99% identity to SEQ ID NO: 115, and/or a light chain comprising an amino acid sequence set forth in SEQ ID NO: 112, or a sequence having at least 90%, 95%. %%, 97%, 98%, or 99% identity to SEQ ID NO: 112.

In some embodiments, the anti-CD98 antibody comprises a heavy chain CDR3 domain comprising the amino acid sequence set forth in SEQ ID NO: 97, a heavy chain CDR2 domain comprising the amino acid sequence set forth in SEQ ID NO: 104, and a heavy chain CDR1 domain comprising the amino acid sequence set forth in SEQ ID NO: 79; and a light chain CDR3 domain comprising the amino acid sequence set forth in SEQ ID NO: 102, a light chain CDR2 domain comprising the amino acid sequence set forth in SEQ ID NO: 45, and a light chain CDR1 domain comprising the amino acid sequence set forth in SEQ ID NO: 83. In yet another embodiment, the anti-CD98 antibody comprises a heavy chain variable region comprising the amino acid sequence set forth in SEQ ID NO: 118, and a light chain variable region comprising the amino acid sequence set forth in SEQ ID NO: 117.

In some embodiments, an anti-CD98 antibody comprises an amino acid sequence set forth in SEQ ID NO: 118, or a sequence having at least 90%, 95%, 96%, 97%, 98%, or 99% identity to SEQ ID NO: 118, and/or a light chain comprising an amino acid sequence set forth in SEQ ID NO: 117, or a sequence having at least 90%, 95%, 96%, 97%, 98%, or 99% identity to SEQ ID NO: 117.

In some embodiments, the antibody comprises an anti-human CD98 (hCD98) antibody comprising a heavy chain comprising the amino acid sequence set forth in SEQ ID NO: 158, and a light chain comprising the amino acid sequence set forth in SEQ ID NO: 159.

In some embodiments, the antibody comprises an anti-human CD98 (hCD98) antibody comprising a heavy chain comprising the amino acid sequence set forth in SEQ ID NO: 160, and a light chain comprising the amino acid sequence set forth in SEQ ID NO: 161.

In some embodiments, the antibody comprises an anti-human CD98 (hCD98) antibody comprising a heavy chain comprising the amino acid sequence set forth in SEQ ID NO: 162, and a light chain comprising the amino acid sequence set forth in SEQ ID NO: 163.

In some embodiments, the antibody comprises an anti-human CD98 (hCD98) antibody comprising a heavy chain comprising the amino acid sequence set forth in SEQ ID NO: 164, and a light chain comprising the amino acid sequence set forth in SEQ ID NO: 165.

In some embodiments, the anti-CD98 antibody, or antigen binding portion thereof, is an IgG isotype. In some embodiments, the antibody, or antigen binding portion thereof, is an IgG1 or an IgG4 isotype.

In other embodiments, the anti-CD98 antibody, or antigen binding portion thereof, has a K₀ of 1.5×10⁻⁸ or less as determined by surface plasmon resonance.

In some embodiments, the antibody, or antigen-binding portion thereof, binds cyno CD98.

In other embodiments, the anti-CD98 antibody, or antigen binding portion thereof, has a dissociation constant (K_{D}) to CD98 selected from the group consisting of: at most about 10⁻⁷ M; at most about 10⁻⁸ M; at most about 10⁻⁹ M: at most about 10⁻¹⁰ M; at most about 10⁻¹¹ M; at most about 10⁻¹² M; and at most 10⁻¹³ M.

In some embodiments, the anti-CD98 antibody, or antigen binding portion thereof, comprises a heavy-chain immunoglobulin constant domain of a human IgM constant domain, a human IgG1 constant domain, a human IgG2 constant domain, a human IgG3 constant domain, a human IgG4 constant domain, a human IgA constant domain, or a human IgE constant domain.

In other embodiments, the heavy chain immunoglobulin constant region domain is a human IgG1 constant domain. In some embodiments, the human IgG1 constant domain comprises an amino acid sequence of SEQ ID NO: 154 or SEQ ID NO: 155.

In some embodiments, the anti-CD98 antibody, or antigen binding portion thereof, is an IgG1 antibody and comprises a human Ig kappa constant domain or a human Ig lambda constant domain.

In other embodiments, the anti-CD98 antibody, or antigen binding portion thereof, competes with the antibody, or antigen binding portion thereof, of any one of the antibodies described herein. e.g., huAb102, huAb104, huAb108, and huAb110.

In one aspect, the invention comprises a pharmaceutical composition comprising an anti-CD98 antibody, or antigen binding portion thereof, e.g., huAb102, huAb104, huAb108, and huAb110, and a pharmaceutically acceptable carrier.

The invention also provides, in certain embodiments, isolated nucleic acids encoding an antibodies, or antigen binding portions thereof, like that described herein.

In other embodiments, the invention includes an anti-hCD98 antibody, or antigen binding portion thereof, comprising a heavy chain CDR set (CDR1, CDR2, and CDR3) selected from the group consisting of SEQ ID NOs: 16, 87, and 17; 16, 90 and 17; 79.92, and 97; and 79, 104, and 97, and a light chain CDR set (CDR1, CDR2, and CDR3) selected from the group consisting of SEQ ID NOs: 13, 7, and 19; 83, 45, and 95; and 83.45, and 102. In some embodiments, the anti-CD98 antibodies, or antigen binding portions thereof, comprises a heavy chain constant region comprising the amino acid sequence set forth in SEQ ID NO: 108 and/or a light chain constant region comprising the amino acid sequence set forth in SEQ ID NO: 107. In some embodiments, the anti-CD98 antibody, or antigen binding portion thereof, comprises a heavy chain constant region comprising the amino acid sequence set forth in SEQ ID NO: 110 and/or a light chain constant region comprising the amino acid sequence set forth in SEQ ID NO: 107. In some embodiments, the anti-CD98 antibody, or antigen binding portion thereof, comprises a heavy chain constant region comprising the amino acid sequence set forth in SEQ ID NO: 115 and/or a light chain constant region comprising the amino acid sequence set forth in SEQ ID NO: 112. In some embodiments, the anti-CD98 antibody, or antigen binding portion thereof, comprises a heavy chain constant region comprising the amino acid sequence set forth in SEQ ID NO: 118 and/or a light chain constant region comprising the amino acid sequence set forth in SEQ ID NO: 117.

In other embodiments, the invention includes an anti-hCD98 antibody comprising a heavy chain CDR set (CDR1, CDR2, and CDR3) selected from the group consisting of SEQ ID NOs: 16, 87, and 17; 16, 90 and 17; 79, 92, and 97; and 79, 104, and 97, and a light chain CDR set (CDR1, CDR2, and CDR3) selected from the group consisting of SEQ ID NOs: 13, 7, and 19; 83, 45, and 95; and 83, 45, and 102. In some embodiments, the anti-CD98 antibody comprises a heavy chain constant region comprising the amino acid sequence set forth in SEQ ID NO: 108 and/or a light chain constant region comprising the amino acid sequence set forth in SEQ ID NO: 107. In some embodiments, the anti-CD98 antibody comprises a heavy chain constant region comprising the amino acid sequence set forth in SEQ ID NO: 110 and/or a light chain constant region comprising the amino acid sequence set forth in SEQ ID NO: 107. In some embodiments, the anti-CD98 antibody comprises a heavy chain constant region comprising the amino acid sequence set forth in SEQ ID NO: 115 and/or a light chain constant region comprising the amino acid sequence set forth in SEQ ID NO: 112. In some embodiments, the anti-CD98 antibody comprises a heavy chain constant region comprising the amino acid sequence set forth in SEQ ID NO: 118 and/or a light chain constant region comprising the amino acid sequence set forth in SEQ ID NO: 117.

In some embodiments of the invention, the anti-CD98 antibody, or antigen binding portion thereof, comprises a heavy chain immunoglobulin constant domain selected from the group consisting of a human IgG constant domain, a human IgM constant domain, a human IgE constant domain, and a human IgA constant domain. In some embodiments, the IgG constant domain is selected from the group consisting of an IgG1 constant domain, an IgG2 constant domain, an IgG3 constant domain, and an IgG4 constant domain. In other embodiments, the antibody is a multispecific antibody.

In other embodiments of the invention, an antigen binding portion of an antibody is, for example, a Fab, a Fab′, a F(ab′)₂, a Fv, a disulfide linked Fv, an scFv, a single domain antibody, and a diabody.

In some embodiments, an anti-CD98 antibody of the invention is an IgG having four polypeptide chains which are two heavy chains and two light chains.

In another embodiment, the anti-CD98 antibody, or antigen binding portion thereof, is conjugated to an auristatin. In another embodiment, the anti-CD98 antibody, or antigen binding portion thereof, is conjugated to a Bcl-xL inhibitor.

In yet other embodiments of the invention, the anti-CD98 antibody, or antigen binding portion thereof, is conjugated to an imaging agent. In certain embodiments of the invention, the imaging agent is selected from the group consisting of a radiolabel, an enzyme, a fluorescent label, a luminescent label, a bioluminescent label, a magnetic label, and biotin. In other embodiments of the invention, the radiolabel is indium. In yet other embodiments, the invention includes a pharmaceutical composition comprising the antibody, or antigen binding portion thereof, and a pharmaceutically acceptable carrier.

The invention also includes, in some embodiments, an anti-CD98 antibody drug conjugate (ADC) comprising the anti-CD98 antibody, or antigen binding portion thereof, described herein, conjugated to at least one drug. In certain embodiments, the anti-CD98 antibody is conjugated to a Bcl-xL inhibitor to form an anti-hCD98 ADC.

In some embodiments, an anti-CD98 ADC of the invention comprises an IgG antibody having four polypeptide chains which are two heavy chains and two light chains.

In one embodiment of the invention, at least one drug is selected from the group consisting of an anti-apoptotic agent, a mitotic inhibitor, an anti-tumor antibiotic, an immunomodulating agent, a nucleic acid for gene therapy, an alkylating agent, an anti-angiogenic agent, an anti-metabolite, a boron-containing agent, a chemoprotective agent, a hormone agent, an anti-hormone agent, a corticosteroid, a photoactive therapeutic agent, an oligonucleotide, a radionuclide agent, a radiosensitizer, a topoisomerase inhibitor, and a kinase inhibitor. In certain embodiments, the mitotic inhibitor is a dolastatin, an auristatin, a maytansinoid, and a plant alkaloid. In certain embodiments, the drag is a dolastatin, an auristatin, a maytansinoid, and a plant alkaloid. An example of an auristatin is monomethylaurisalin F (MMAF) or monomethyauristatin E (MMAE). Examples of maytansinoids include, but are not limited to, DM1, DM2, DM3, and DM4. In certain embodiments, the anti-tumor antibiotic is selected from the group consisting of an actinomycine, an anthracycline, a calicheamicin, and a duocarmycin. In certain embodiments, the actinomycine is a pyrrolobenzodiazepine (PBD).

The invention also includes, in some embodiments, an ADC comprising an anti-CD98 antibody conjugated to a Bcl-xL inhibitor wherein the anti-CD98 antibody comprises a heavy chain variable region comprising a CDR3 domain comprising the amino acid sequence set forth in SEQ ID NO: 17, a heavy chain CDR2 domain comprising the amino acid sequence set forth in SEQ ID NO: 87, and a heavy chain CDR1 domain comprising the amino acid sequence set forth in SEQ ID NO: 16; and a light chain CDR3 domain comprising the amino acid sequence set forth in SEQ ID NO: 19, a light chain CDR2 domain comprising the amino acid sequence set forth in SEQ ID NO: 7, and a light chain CDR1 domain comprising the amino acid sequence set forth in SEQ ID NO: 13. In yet another embodiment, the anti-CD98 antibody, or antigen binding portion thereof, comprises a heavy chain variable region comprising the amino acid sequence set forth in SEQ ID NO: 108, and a light chain variable region comprising the amino acid sequence set forth in SEQ ID NO: 107.

The invention also includes, in some embodiments, an ADC comprising an anti-CD98 antibody conjugated to a Bcl-xL inhibitor, wherein the anti-CD98 antibody comprises a heavy chain CDR3 domain comprising the amino acid sequence set forth in SEQ ID NO: 17, a heavy chain CDR2 domain comprising the amino acid sequence set forth in SEQ ID NO: 90, and a heavy chain CDR1 domain comprising the amino acid sequence set forth in SEQ ID NO: 16; and a light chain CDR3 domain comprising the amino acid sequence set forth in SEQ ID NO: 19, a light chain CDR2 domain comprising the amino acid sequence set forth in SEQ ID NO: 7, and a light chain CDR1 domain comprising the amino acid sequence set forth in SEQ ID NO: 13. In yet another embodiment, the antibody, or antigen binding portion thereof, comprises a heavy chain variable region comprising the amino acid sequence set forth in SEQ ID NO: 110, and a light chain variable region comprising the amino acid sequence set forth in SEQ ID NO: 107.

The invention also includes, in some embodiments, an ADC comprising an anti-CD98 antibody conjugated to a Bcl-xL inhibitor, wherein the antibody comprises a heavy chain CDR3 domain comprising the amino acid sequence set forth in SEQ ID NO: 97, a heavy chain CDR2 domain comprising the amino acid sequence set forth in SEQ ID NO: 92, and a heavy chain CDR1 domain comprising the amino acid sequence set forth in SEQ ID NO: 79; and a light chain CDR3 domain comprising the amino acid sequence set forth in SEQ ID NO: 95, a light chain CDR2 domain comprising the amino acid sequence set forth in SEQ ID NO: 45, and a light chain CDR1 domain comprising the amino acid sequence set forth in SEQ ID NO: 83. In yet another embodiment, the anti-CD98 antibody, or antigen binding portion thereof, comprises a heavy chain variable region comprising the amino acid sequence set forth in SEQ ID NO: 115, and a light chain variable region comprising the amino acid sequence set forth in SEQ ID NO: 112.

The invention also includes, in some embodiments, an ADC comprising an anti-CD98 antibody conjugated to a Bcl-xL inhibitor, wherein the antibody comprises a heavy chain CDR3 domain comprising the amino acid sequence set forth in SEQ ID NO: 97, a heavy chain CDR2 domain comprising the amino acid sequence set forth in SEQ ID NO: 104, and a heavy chain CDR1 domain comprising the amino acid sequence set forth in SEQ ID NO: 79; and a light chain CDR3 domain comprising the amino acid sequence set forth in SEQ ID NO: 102, a light chain CDR2 domain comprising the amino acid sequence set forth in SEQ ID NO: 45, and a light chain CDR1 domain comprising the amino acid sequence set forth in SEQ ID NO: 83. In yet another embodiment, the anti_CD98 antibody, or antigen binding portion thereof, comprises a heavy chain variable region comprising the amino acid sequence set forth in SEQ ID NO: 118, and a light chain variable region comprising the amino acid sequence set forth in SEQ ID NO: 117.

The invention also includes, in some embodiments, an ADC comprising an anti-CD98 antibody conjugated to at least one drug (including, but not limited to, a Bcl-xL inhibitor), wherein between 1 to 8 molecules of the drug are conjugated to the antibody. In one embodiment, 1 to 4 molecules of the drug are conjugated to the antibody of the ADC. In one embodiment, 2 to 4 molecules of the drug are conjugated to the antibody of the ADC.

The invention also includes, in some embodiments, an ADC comprising an anti-CD98 antibody conjugated to at least one drug, wherein the drug is conjugated via a maleimidocaproyl, valine-citrulline linker. In a further embodiment, the drug is conjugated to the antibody via a maleimidocaproyl, valine-citrulline, p-aminobenzyloxycarbamyl (PABA) linker.

The invention also includes, in some embodiments, an ADC comprising an anti-CD98 IgG1 antibody covalently linked to a Bcl-xL inhibitor via a linker. In certain embodiments, the antibody comprises a heavy chain variable region comprising the amino acid sequence set forth in SEQ ID NO: 108, 110, 115, or 118, and comprises a light chain variable region comprising the amino acid sequence set forth in SEQ ID NO: 107, 112, or 117. In certain embodiments, 1 to 4 molecules of a Bcl-xL inhibitor are linked to the antibody. In certain embodiments, 2 to 4 molecules of the Bcl-xL inhibitor are linked to the anti-CD98 antibody.

The invention also includes, in some embodiments, an CD98-directed ADC comprising an IgG1 antibody specific for human CD98, a Bcl-xL inhibitor, and a linker that covalently attaches the Bcl-xL inhibitor to the antibody. In certain embodiments, the anti-CD98 antibody comprises a heavy chain CDR3 domain comprising the amino acid sequence set forth in SEQ ID NO: 17, a heavy chain CDR2 domain comprising the amino acid sequence set forth in SEQ ID NO: 87, and a heavy chain CDR1 domain comprising the amino acid sequence set forth in SEQ ID NO: 16; and a light chain CDR3 domain comprising the amino acid sequence set forth in SEQ ID NO: 19, a light chain CDR2 domain comprising the amino acid sequence set forth in SEQ ID NO: 7, and a light chain CDR1 domain comprising the amino acid sequence set forth in SEQ ID NO: 13. In yet another embodiment, the anti-CD98 antibody comprises a heavy chain variable region comprising the amino acid sequence set forth in SEQ ID NO: 108, and a light chain variable region comprising the amino acid sequence set forth in SEQ ID NO: 107. In other embodiments, the antibody comprises a heavy chain CDR3 domain comprising the amino acid sequence set forth in SEQ ID NO: 17, a heavy chain CDR2 domain comprising the amino acid sequence set forth in SEQ ID NO: 90, and a heavy chain CDR1 domain comprising the amino acid sequence set forth in SEQ ID NO: 16; and a light chain CDR3 domain comprising the amino acid sequence set forth in SEQ ID NO: 19, a light chain CDR2 domain comprising the amino acid sequence set forth in SEQ ID NO: 7, and a light chain CDR1 domain comprising the amino acid sequence set forth in SEQ ID NO: 13. In yet another embodiment, the anti_CD98 antibody, or antigen binding portion thereof, comprises a heavy chain variable region comprising the amino acid sequence set forth in SEQ ID NO: 110, and a light chain variable region comprising the amino acid sequence set forth in SEQ ID NO: 107. In other embodiments, the anti-CD98 antibody comprises a heavy chain CDR3 domain comprising the amino acid sequence set forth in SEQ ID NO: 97, a heavy chain CDR2 domain comprising the amino acid sequence set forth in SEQ ID NO: 92, and a heavy chain CDR1 domain comprising the amino acid sequence set forth in SEQ ID NO: 79: and a light chain CDR3 domain comprising the amino acid sequence set forth in SEQ ID NO: 95, a light chain CDR2 domain comprising the amino acid sequence set forth in SEQ ID NO: 45, and a light chain CDR1 domain comprising the amino acid sequence set forth in SEQ ID NO: 83. In yet another embodiment the anti-CD98 antibody comprises a heavy chain variable region comprising the amino acid sequence set forth in SEQ ID NO: 115, and a light chain variable region comprising the amino acid sequence set forth in SEQ ID NO: 112. In other embodiments, the anti-CD98 antibody comprises a heavy chain CDR3 domain comprising the amino acid sequence set forth in SEQ ID NO: 97, a heavy chain CDR2 domain comprising the amino acid sequence set forth in SEQ ID NO: 104, and a heavy chain CDR1 domain comprising the amino acid sequence set forth in SEQ ID NO: 79; and a light chain CDR3 domain comprising the amino acid sequence set forth in SEQ ID NO: 102, a light chain CDR2 domain comprising the amino acid sequence set forth in SEQ ID NO: 45, and a light chain CDR1 domain comprising the amino acid sequence set forth in SEQ ID NO: 83. In yet another embodiment, the anti-CD98 antibody comprises a heavy chain variable region comprising the amino acid sequence set forth in SEQ ID NO: 118, and a light chain variable region comprising the amino acid sequence set forth in SEQ ID NO: 117.

In yet other embodiments, the invention includes a pharmaceutical composition comprising an ADC mixture comprising a plurality of the ADC described herein, and a pharmaceutically acceptable carrier. In certain embodiments, the ADC mixture has an average drug to antibody ratio (DAR) of 2 to 4. In other embodiments the ADC mixture comprises ADCs each having a DAR of 2 to 8. In certain embodiments, the ADC mixture has an average drug to antibody (DAR) of about 2.4 to about 3.6.

In certain embodiments, the invention includes methods for treating a subject having cancer, comprising administering the pharmaceutical composition described herein to the subject such that the subject having cancer is treated. In one embodiment, the cancer is selected from the group consisting of breast cancer, lung cancer, a glioblastoma, prostate cancer, pancreatic cancer, colon cancer, head and neck cancer, kidney cancer, and a hematological cancer such as multiple myeloma, acute myeloid leukemia, or lymphoma. In one embodiment, the cancer is selected from the group consisting of breast cancer, ovarian cancer, lung cancer, a glioblastoma, prostate cancer, pancreatic cancer, colon cancer, colorectal cancer, head and neck cancer, mesothelioma, kidney cancer, squamous cell carcinoma, triple negative breast cancer, small cell lung cancer, and non-small cell lung cancer. In one embodiment, the cancer is breast cancer. In one embodiment, the cancer is lung cancer. In one embodiment, the cancer is prostate cancer. In one embodiment, the cancer is pancreatic cancer. In one embodiment, the cancer is colon cancer. In one embodiment, the cancer is head and neck cancer. In one embodiment, the cancer is kidney cancer. In one embodiment, the cancer is a hematological cancer. In certain embodiments, the hematological cancer is multiple myeloma. In certain embodiments, the hematological cancer is acute myeloid leukemia. In other embodiments, the hematological cancer is lymphoma. In one embodiment, the cancer is colorectal cancer. In one embodiment, the cancer is mesothelioma. In one embodiment, the cancer is squamous cell carcinoma. In one embodiment, the cancer is triple negative breast cancer. In one embodiment, the cancer is non-small cell lung cancer. In certain embodiments, the squamous cell carcinoma is squamous lung cancer or squamous head and neck cancer. In certain embodiments, the cancer is characterized as having EGFR overexpression. In other embodiments, the cancer is characterized as having an activating EGFR mutation, e.g, a mutation(s) that activates the EGFR signaling pathway- and/or mutation(s) that lead to overexpression of the EGFR protein. In specific exemplary embodiments, the activating EGFR mutation may be a mutation in the EGFR gene. In particular embodiments, the activating EGFR mutation is an exon 19 deletion mutation, a single-point substitution mutation L858R in exon 21, a T790M point mutation, and/or combinations thereof. In yet another embodiment, the cancer contains amplifications of CD98 or overexpresses CD98. In certain embodiments, the cancer is characterized as having CD98 overexpression. In certain embodiments, the cancer is characterized as having CD98 amplification.

The invention further includes, in certain embodiments, methods for inhibiting or decreasing solid tumor growth in a subject having a solid tumor, comprising administering the pharmaceutical composition described herein to the subject having the solid tumor, such that the solid tumor growth is inhibited or decreased. In certain embodiments, the solid tumor is characterized as having CD98 overexpression. In certain embodiments, the solid tumor is characterized as having CD98 amplification.

In one embodiment of the invention, the invention provides for methods for inhibiting or decreasing solid tumor growth in a subject having a solid tumor, comprising administering to the subject having the solid tumor an effective amount of the antibody or ADC described herein, such that the solid tumor growth is inhibited or decreased.

In certain embodiments, the solid tumor is an CD98 expressing solid tumor. In other embodiments, the solid tumor is a non-small cell lung carcinoma or a glioblastoma. In other embodiments, the solid tumor is a squamous cell carcinoma.

In one embodiment of the invention, the invention provides for a method for treating a subject having cancer, comprising administering an effective amount of an ADC comprising an anti-CD98 antibody conjugated to at least one Bcl-xL inhibitor, wherein the anti-CD98 antibody is an IgG isotype and comprises a heavy chain CDR3 domain comprising the amino acid sequence set forth in SEQ ID NO: 17, a heavy chain CDR2 domain comprising the amino acid sequence set forth in SEQ ID NO: 87, and a heavy chain CDR1 domain comprising the amino acid sequence set forth in SEQ ID NO: 16; and a light chain CDR3 domain comprising the amino acid sequence set forth in SEQ ID NO: 19, a light chain CDR2 domain comprising the amino acid sequence set forth in SEQ ID NO: 7, and a light chain CDR1 domain comprising the amino acid sequence set forth in SEQ ID NO: 13. In yet another embodiment, the anti-CD98 antibody comprises a heavy chain variable region comprising the amino acid sequence set forth in SEQ ID NO: 108, and a light chain variable region comprising the amino acid sequence set forth in SEQ ID NO: 107.

In one embodiment of the invention, the invention provides for a method for treating a subject having cancer, comprising administering an effective amount of an ADC comprising an anti-CD98 antibody conjugated to at least one Bcl-xL inhibitor, wherein the anti-CD98 antibody, or antigen binding portion thereof, is an IgG isotype and comprises a heavy chain CDR3 domain comprising the amino acid sequence set forth in SEQ ID NO: 17, a heavy chain CDR2 domain comprising the amino acid sequence set forth in SEQ ID NO: 90, and a heavy chain CDR1 domain comprising the amino acid sequence set forth in SEQ ID NO: 16; and a light chain CDR3 domain comprising the amino acid sequence set forth in SEQ ID NO: 19, a light chain CDR2 domain comprising the amino acid sequence set forth in SEQ ID NO: 7, and a light chain CDR1 domain comprising the amino acid sequence set forth in SEQ ID NO: 13. In yet another embodiment, the anti-CD98 antibody, or antigen binding portion thereof, comprises a heavy chain variable region comprising the amino acid sequence set forth in SEQ ID NO: 110, and a light chain variable region comprising the amino acid sequence set forth in SEQ ID NO: 107.

In one embodiment of the invention, the invention provides for a method for treating a subject having cancer, comprising administering an effective amount of an ADC comprising an anti-CD98 antibody conjugated to at least one Bcl-xL inhibitor, wherein the anti-CD98 antibody, or antigen binding portion thereof, is an IgG isotype and comprises a heavy chain CDR3 domain comprising the amino acid sequence set forth in SEQ ID NO: 97, a heavy chain CDR2 domain comprising the amino acid sequence set forth in SEQ ID NO: 92, and a heavy chain CDR1 domain comprising the amino acid sequence set forth in SEQ ID NO: 79; and a light chain CDR3 domain comprising the amino acid sequence set forth in SEQ ID NO: 95, a light chain CDR2 domain comprising the amino acid sequence set forth in SEQ ID NO: 45, and a light chain CDR1 domain comprising the amino acid sequence set forth in SEQ ID NO: 83. In yet another embodiment, the anti-CD98 antibody, or antigen binding portion thereof, comprises a heavy chain variable region comprising the amino acid sequence set forth in SEQ ID NO: 115, and a light chain variable region comprising the amino acid sequence set forth in SEQ ID NO: 112.

In one embodiment of the invention, the invention provides for a method for treating a subject having cancer, comprising administering an effective amount of an ADC comprising an anti-CD98 antibody conjugated to at least one Bcl-xL inhibitor, wherein the anti-CD98 antibody, or antigen binding portion thereof, is an IgG isotype and comprises a heavy chain CDR3 domain comprising the amino acid sequence set forth in SEQ ID NO: 97, a heavy chain CDR2 domain comprising the amino acid sequence set forth in SEQ ID NO: 104, and a heavy chain CDR1 domain comprising the amino acid sequence set forth in SEQ ID NO: 79; and a light chain CDR3 domain comprising the amino acid sequence set forth in SEQ ID NO: 102, a light chain CDR2 domain comprising the amino acid sequence set forth in SEQ ID NO: 45, and a light chain CDR1 domain comprising the amino acid sequence set forth in SEQ ID NO: 83. In yet another embodiment, the anti-CD98 antibody, or antigen binding portion thereof, comprises a heavy chain variable region comprising the amino acid sequence set forth in SEQ ID NO: 118, and a light chain variable region comprising the amino acid sequence set forth in SEQ ID NO: 117.

In certain embodiments, the invention includes methods for treating a subject having cancer, comprising administering the pharmaceutical composition described herein to the subject in combination with an additional agent or additional therapy. In certain embodiments, the additional agent is selected from the group consisting of an anti-PD1 antibody (e.g, pembrolizumab), an anti-PD-L1 antibody (e.g, atezolizumab), an anti-CTLA-4 antibody (e.g, ipilimumab), a MEK inhibitor (e.g, trametinib), an ERK inhibitor, a BRAF inhibitor (e.g, dabrafenib), osimertinib, erlotinib, gefitinib, sorafenib, a CDK9 inhibitor (e.g, dinaciclib), a MCL-1 inhibitor, temozolomide, a Bcl-xL inhibitor, a Bcl-2 inhibitor (e.g. veneloclax), ibrutinib, a mTOR inhibitor (e.g, everolimus), a PI3K inhibitor (e.g. buparlisib), duvelisib, idelalisib, an AKT inhibitor, a HER2 inhibitor (e.g. lapatinib), a taxane (e.g. docetaxel, paclitaxel, nab-paclitaxel), an ADC comprising an auristatin, an ADC comprising a PBD (e.g, rovalpituzumab tesirine), an ADC comprising a maytansinoid (e.g. TDM1), a TRAIL agonist, a proteasome inhibitor (e.g, bortezomib), and a nicotinamide phosphoribosyltransferase (NAMPT) inhibitor. In certain embodiments, the additional agent is an anti-CTLA-4 antibody (e.g., ipilimumab). In certain embodiments, the additional agent is ibrutinib. In certain embodiments, the additional agent is duvelisib. In certain embodiments, the additional agent is idelalisib. In certain embodiments, the additional agent is venetoclax. In certain embodiments, the additional agent is temozolomide.

The invention also provides, in certain embodiments, isolated nucleic acids encoding an antibodies, or antigen binding portions thereof, like that described herein. Further, the invention includes a vector comprising the nucleic acid, and a host cell, e.g., a prokaryotic or a eukaryotic cell (e.g., animal cell, a protest cell, a plant cell, and a fungal cell) comprising the vector. In embodiment of the invention, the animal cell is selected from the group consisting of a mammalian cell, an insect cell, and an avian cell. In one embodiment, the mammalian cell is selected from the group consisting of a CHO cell, a COS cell, and an Sp2/0 cell.

In certain embodiments, the invention features anti-hCD98 Antibody Drug Conjugates (ADC) comprising an anti-hCD98 antibody conjugated to a Bcl-xL inhibitor, wherein the antibody comprises a heavy chain CDR3 domain comprising the amino acid sequence set forth in SEQ ID NO: 17, a heavy chain CDR2 domain comprising the amino acid sequence set forth in SEQ ID NO: 87, and a heavy chain CDR1 domain comprising the amino acid sequence set forth in SEQ ID NO: 16; and a light chain CDR3 domain comprising the amino acid sequence set forth in SEQ ID NO: 19, a light chain CDR2 domain comprising the amino acid sequence set forth in SEQ ID NO: 7, and a light chain CDR1 domain comprising the amino acid sequence set forth in SEQ ID NO: 13. In yet another embodiment, the anti-CD98 antibody, or antigen binding portion thereof, comprises a heavy chain variable region comprising the amino acid sequence set forth in SEQ ID NO: 108, and a light chain variable region comprising the amino acid sequence set forth in SEQ ID NO: 107.

In other embodiments, the invention features anti-hCD98 Antibody Drug Conjugates (ADC) comprising an anti-hCD98 antibody conjugated to a Bcl-xL inhibitor, wherein the antibody comprises a heavy chain CDR3 domain comprising the amino acid sequence set forth in SEQ ID NO: 17, a heavy chain CDR2 domain comprising the amino acid sequence set forth in SEQ ID NO: 90, and a heavy chain CDR1 domain comprising the amino acid sequence set forth in SEQ ID NO: 16; and a light chain CDR3 domain comprising the amino acid sequence set forth in SEQ ID NO: 19, a light chain CDR2 domain comprising the amino acid sequence set forth in SEQ ID NO: 7, and a light chain CDR1 domain comprising the amino acid sequence set forth in SEQ ID NO: 13. In yet another embodiment, the anti-CD98 antibody, or antigen binding portion thereof, comprises a heavy chain variable region comprising the amino acid sequence set forth in SEQ ID NO: 110, and a light chain variable region comprising the amino acid sequence set forth in SEQ ID NO: 107.

In other embodiments, the invention features anti-hCD98 Antibody Drug Conjugates (ADC) comprising an anti-hCD98 antibody conjugated to a Bcl-xL inhibitor, wherein the antibody comprises a heavy chain CDR3 domain comprising the amino acid sequence set forth in SEQ ID NO: 97, a heavy chain CDR2 domain comprising the amino acid sequence set forth in SEQ ID NO: 92, and a heavy chain CDR1 domain comprising the amino acid sequence set forth in SEQ ID NO: 79; and a light chain CDR3 domain comprising the amino acid sequence set forth in SEQ ID NO: 95, a light chain CDR2 domain comprising the amino acid sequence set forth in SEQ ID NO: 45, and a light chain CDR1 domain comprising the amino acid sequence set forth in SEQ ID NO: 83. In yet another embodiment, the anti-CD98 antibody, or antigen binding portion thereof, comprises a heavy chain variable region comprising the amino acid sequence set forth in SEQ ID NO: 115, and a light chain variable region comprising the amino acid sequence set forth in SEQ ID NO: 112.

In other embodiments, the invention features anti-hCD98 Antibody Drug Conjugates (ADC) comprising an anti-hCD98 antibody conjugated to a Bcl-xL inhibitor, wherein the antibody comprises a heavy-chain CDR3 domain comprising the amino acid sequence set forth in SEQ ID NO: 97, a heavy chain CDR2 domain comprising the amino acid sequence set forth in SEQ ID NO: 104, and a heavy chain CDR1 domain comprising the amino acid sequence set forth in SEQ ID NO: 79; and a light chain CDR3 domain comprising the amino acid sequence set forth in SEQ ID NO: 102, a light chain CDR2 domain comprising the amino acid sequence set forth in SEQ ID NO: 45, and a light chain CDR1 domain comprising the amino acid sequence set forth in SEQ ID NO: 83. In yet another embodiment, the anti-CD98 antibody, or antigen binding portion thereof, comprises a heavy chain variable region comprising the amino acid sequence set forth in SEQ ID NO: 118, and a light chain variable region comprising the amino acid sequence set forth in SEQ ID NO: 117.

In yet another embodiment, the anti-CD98 antibody comprises an IgG heavy chain immunoglobulin constant domain. In still another embodiment, the IgG is an IgG1 or an IgG4 heavy chain immunoglobulin constant domain.

In one embodiment, the invention includes an ADC comprising an anti-hCD98 antibody conjugated to an auristatin, wherein the auristatin is monomethylaurisatin F (MMAF) or monomethyauristatin E (MMAE). In one embodiment, the invention includes an ADC, wherein the auristatin is monomethylaurisatin F (MMAF). In one embodiment the invention includes an ADC, wherein the auristatin is monomethyauristatin E (MMAE). In still another embodiment of the invention, the anti-CD98 antibody is covalently linked to the auristatin by a linker comprising maleimidocaproyl, valine-citrulline, p-aminobenzylalcohol (mc-vc-PABA).

In one embodiment, the invention includes an ADC comprising an anti-CD98 and a radiolabel, e.g, indium.

In one embodiment, an anti-CD98 antibody described herein is covalently linked to at least one pyrrolobenzodiazepine (PBD). In certain embodiments, the anti-CD98 antibody disclosed herein is linked to a PBD as described in FIG. 4 (i.e., SGD-1882).

In some embodiments, the invention features pharmaceutical compositions comprising the ADC described herein, and a pharmaceutically acceptable carrier. In certain embodiments, the invention features pharmaceuticals composition comprising an ADC mixture comprising the ADC described herein, wherein the average drug to antibody ratio (DAR) range in the ADC mixture is 2 to 4. In certain embodiments, the average drug to antibody ratio (DAR) range in the ADC mixture is 2.4 to 3.6.

In one embodiment, the invention features pharmaceutical compositions comprising an ADC mixture comprising anti-hCD98 Antibody Drug Conjugates (ADCs), and a pharmaceutically acceptable carrier, wherein the ADC mixture has an average Drug to Antibody Ratio (DAR) of 2 to 4, and wherein said ADC comprises a Bcl-xL inhibitor conjugated to an anti-hCD98 antibody comprising a heavy chain CDR3 domain comprising the amino acid sequence set forth in SEQ ID NO: 17, a heavy chain CDR2 domain comprising the amino acid sequence set forth in SEQ ID NO: 87, and a heavy chain CDR1 domain comprising the amino acid sequence set forth in SEQ ID NO: 16; and a light chain CDR3 domain comprising the amino acid sequence set forth in SEQ ID NO: 19, a light chain CDR2 domain comprising the amino acid sequence set forth in SEQ ID NO: 7, and a light chain CDR1 domain comprising the amino acid sequence set forth in SEQ ID NO: 13. In yet another embodiment the anti-CD98 antibody comprises a heavy chain variable region comprising the amino acid sequence set forth in SEQ ID NO: 108, and a light chain variable region comprising the amino acid sequence set forth in SEQ ID NO: 107.

In another embodiment, the invention features pharmaceutical compositions comprising an ADC mixture comprising anti-hCD98 Antibody Drug Conjugates (ADCs), and a pharmaceutically acceptable carrier, wherein the ADC mixture has an average Drug to Antibody Ratio (DAR) of 2 to 4, and wherein said ADC comprises a Bcl-xL inhibitor conjugated to an anti-hCD98 antibody comprising a heavy chain CDR3 domain comprising the amino acid sequence set forth in SEQ ID NO: 17, a heavy chain CDR2 domain comprising the amino acid sequence set forth in SEQ ID NO: 90, and a heavy chain CDR1 domain comprising the amino acid sequence set forth in SEQ ID NO: 16; and a light chain CDR3 domain comprising the amino acid sequence set forth in SEQ ID NO: 19, a light chain CDR2 domain comprising the amino acid sequence set forth in SEQ ID NO: 7, and a light chain CDR1 domain comprising the amino acid sequence set forth in SEQ ID NO: 13. In yet another embodiment, the anti-CD98 antibody comprises a heavy chain variable region comprising the amino acid sequence set forth in SEQ ID NO: 110, and a light chain variable region comprising the amino acid sequence set forth in SEQ ID NO: 107.

In yet another embodiment, the invention features pharmaceutical compositions comprising an ADC mixture comprising anti-hCD98 Antibody Drug Conjugates (ADCs), and a pharmaceutically acceptable carrier, wherein the ADC mixture has an average Drug to Antibody Ratio (DAR) of 2 to 4, and wherein said ADC comprises a Bcl-xL inhibitor conjugated to an anti-hCD98 antibody-comprising a heavy chain CDR2 domain comprising the amino acid sequence set forth in SEQ ID NO: 92, and a heavy chain CDR1 domain comprising the amino acid sequence set forth in SEQ ID NO: 79; and a light chain CDR3 domain comprising the amino acid sequence set forth in SEQ ID NO: 95, a light chain CDR2 domain comprising the amino acid sequence set forth in SEQ ID NO: 45, and a light chain CDR1 domain comprising the amino acid sequence set forth in SEQ ID NO: 83. In yet another embodiment, the anti-CD98 antibody comprises a heavy chain variable region comprising the amino acid sequence set forth in SEQ ID NO: 115, and a light chain variable region comprising the amino acid sequence set forth in SEQ ID NO: 112.

In a further embodiment, the invention features pharmaceutical compositions comprising an ADC mixture comprising anti-hCD98 Antibody Drug Conjugates (ADCs), and a pharmaceutically acceptable carrier, wherein the ADC mixture has an average Drug to Antibody Ratio (DAR) of 2 to 4, and wherein said ADC comprises a Bcl-xL inhibitor conjugated to an anti-hCD98 antibody comprising a heavy chain CDR2 domain comprising the amino acid sequence set forth in SEQ ID NO: 104, and a heavy chain CDR1 domain comprising the amino acid sequence set forth in SEQ ID NO: 79; and a light chain CDR3 domain comprising the amino acid sequence set forth in SEQ ID NO: 102, a light chain CDR2 domain comprising the amino acid sequence set forth in SEQ ID NO: 45, and a light chain CDR1 domain comprising the amino acid sequence set forth in SEQ ID NO: 83. In yet another embodiment, the anti-CD98 antibody comprises a heavy chain variable region comprising the amino acid sequence set forth in SEQ ID NO: 118, and a light chain variable region comprising the amino acid sequence set forth in SEQ ID NO: 117.

In other embodiments of the invention, the antibody comprises an IgG heavy chain immunoglobulin constant domain. In further embodiments, the invention includes an antibody having an IgG1 or an IgG4 heavy chain immunoglobulin constant domain. In one embodiment, the invention includes an antibody is an IgG1 isotype.

In yet another embodiment, the invention includes antibodies comprising a heavy chain comprising the amino acid sequence set forth in SEQ ID NO: 108, 110, 115, or 118, and a light chain comprising the amino acid sequence of SEQ ID NO: 107 or 112. In one embodiment, the invention features having a Bcl-xL inhibitor which is conjugated to the antibody by a linker.

In one embodiment of the invention, the invention provides methods for treating a subject having cancer, comprising administering a pharmaceutical composition comprising an antibody or ADC described herein to the subject, such that the subject having cancer is treated. In one embodiment, the cancer is selected from the group consisting of breast cancer, ovarian cancer, lung cancer, a glioblastoma, prostate cancer, pancreatic cancer, colon cancer, head and neck cancer, kidney cancer, and a hematological cancer such as multiple myeloma, lymphoma, and acute myeloid leukemia. In one embodiment, the cancer is selected from the group consisting of breast cancer, ovarian cancer, lung cancer, a glioblastoma, prostate cancer, pancreatic cancer, colon cancer, colorectal cancer, head and neck cancer, mesothelioma, kidney cancer, squamous cell carcinoma, triple negative breast cancer, small cell lung cancer, and non-small cell lung cancer. In yet another embodiment, the cancer contains overexpresses CD98. In one embodiment, the squamous cell carcinoma is squamous lung cancer or squamous head and neck cancer. In one embodiment, the cancer is an CD98 overexpressing cancer. In one embodiment, the cancer is breast cancer. In one embodiment, the cancer is lung cancer. In one embodiment, the cancer is prostate cancer. In one embodiment, the cancer is pancreatic cancer. In one embodiment, the cancer is colon cancer. In one embodiment, the cancer is head and neck cancer. In one embodiment, the cancer is kidney cancer. In one embodiment, the cancer is a hematological cancer. In certain embodiments, the hematological cancer is multiple myeloma. In certain embodiments, the hematological cancer is acute myeloid leukemia. In other embodiments, the hematological cancer is lymphoma. In one embodiment, the cancer is colorectal cancer. In one embodiment, the cancer is mesothelioma. In one embodiment, the cancer is squamous cell carcinoma. In one embodiment, the cancer is triple negative breast cancer. In one embodiment, the cancer is non-small cell lung cancer. In certain embodiments, the squamous cell carcinoma is squamous lung cancer or squamous head and neck cancer. In certain embodiments, the cancer is characterized as having EGFR overexpression. In other embodiments, the cancer is characterized as having an activating EGFR mutation, e.g, a mutation(s) that activates the EGFR signaling pathway and/or mutation(s) that lead to overexpression of the EGFR protein. In specific exemplary embodiments, the activating EGFR mutation may be a mutation in the EGFR gene. In particular embodiments, the activating EGFR mutation is an exon 19 deletion mutation, a single-point substitution mutation L858R in exon 21, a T790M point mutation, and/or combinations thereof.

In addition, in certain embodiments, the invention provides methods for inhibiting or decreasing solid tumor growth in a subject having a solid tumor, said method comprising administering the pharmaceutical composition described herein to the subject having the solid tumor, such that the solid tumor growth is inhibited or decreased. In one embodiment, the solid tumor is a non-small cell lung carcinoma or a glioblastoma. In yet another embodiment, the solid tumor is an CD98 overexpressing solid tumor. In yet another embodiment, the solid tumor is an CD98 amplified tumor. In one embodiment, the solid tumor is a non-small cell lung carcinoma having amplified CD98. In one embodiment, the solid tumor is a non-small cell lung carcinoma having CD98 overexpression. In one embodiment, the solid tumor is a glioblastoma having amplified CD98. In one embodiment, the solid tumor is a glioblastoma having CD98 overexpression.

In certain embodiments, the invention provides combination therapies whereby the pharmaceutical compositions described herein are administered to a subject in need thereof, (e.g., a subject having cancer or a solid tumor). The pharmaceutical compositions described herein may be administered at the same time as, prior to, or following administration of an additional agent or additional therapy. In certain embodiments, the additional agent is selected from the group consisting of an anti-PD1 antibody (e.g, pembrolizumab), an anti-PD-L1 antibody (e.g, atezolizumab), an anti-CTLA-4 antibody (e.g, ipilimumab), a MEK inhibitor (e.g, trametinib), an ERK inhibitor, a BRAF inhibitor (e.g, dabrafenib), osimertinib, erlotinib, gefitinib, sorafenib, a CDK9 inhibitor (e.g. dinaciclib), a MCL-1 inhibitor, temozolomide, a Bcl-xL inhibitor, a Bcl-2 inhibitor (e.g. venetoclax), ibrutinib, a mTOR inhibitor (e.g, everolimus), a PI3K inhibitor (e.g, buparlisib), duvelisib, idelalisib, an AKT inhibitor, a HER2 inhibitor (e.g. lapatinib), a taxane (e.g, docetaxel, paclitaxel, nab-paclitaxel), an ADC comprising an auristatin, an ADC comprising a PBD (e.g, rovalpituzumab tesirine), an ADC comprising a maytansinoid (e.g. TDM1), a TRAIL agonist, a proteasome inhibitor (e.g, bortezomib), and a nicotinamide phosphoribosyltransferase (NAMPT) inhibitor. In yet other embodiments, the additional agent is a chemotherapeutic agent. In certain embodiments, the additional therapy is radiation. In other embodiments, the additional agent is ibrutinib (Imbruvica®, Pharmacyclics). In other embodiments, the additional agent is duvelisib. In other embodiments, the additional agent is idelalisib (Zydelig®, Gilead Sciences, Inc.). In other embodiments, the additional agent is venetoclax (ABT-199/GDC-0199, AbbVie, Inc.). In certain embodiments, the additional agent is an anti-PD1 antibody (e.g., pembrolizumab (Keytruda®) or nivolumab). In certain embodiments, the additional agent is an anti-PD-L1 antibody (e.g, atezolizumab). In certain embodiments, the additional agent is an anti-CTLA-4 antibody (e.g., ipilimumab). In certain embodiments, the additional agent is temozolomide.

In certain embodiments, the invention features a chimeric antigen receptor (CAR) comprising antigen binding regions, e.g, CDRs, of the antibodies described herein or an scFv described herein. In certain embodiments, the invention features a CAR comprising a heavy chain CDR3 domain comprising the amino acid sequence set forth in SEQ ID NO: 17, a heavy chain CDR2 domain comprising the amino acid sequence set forth in SEQ ID NO: 87, and a heavy chain CDR1 domain comprising the amino acid sequence set forth in SEQ ID NO: 16; and a light chain CDR3 domain comprising the amino acid sequence set forth in SEQ ID NO: 19, a light chain CDR2 domain comprising the amino acid sequence set forth in SEQ ID NO: 7, and a light chain CDR1 domain comprising the amino acid sequence set forth in SEQ ID NO: 13. In certain embodiments, the invention features a CAR comprising a heavy chain variable region comprising the amino acid sequence set forth in SEQ ID NO: 108, and a light chain variable region comprising the amino acid sequence set forth in SEQ ID NO: 107.

In other embodiments, the invention features a CAR comprising a heavy chain CDR3 domain comprising the amino acid sequence set forth in SEQ ID NO: 17, a heavy chain CDR2 domain comprising the amino acid sequence set forth in SEQ ID NO: 90, and a heavy chain CDR1 domain comprising the amino acid sequence set forth in SEQ ID NO: 16; and a light chain CDR3 domain comprising the amino acid sequence set forth in SEQ ID NO: 19, a light chain CDR2 domain comprising the amino acid sequence set forth in SEQ ID NO: 7, and a light chain CDR1 domain comprising the amino acid sequence set forth in SEQ ID NO: 13. In other embodiments, the invention features a CAR comprising a heavy chain variable region comprising the amino acid sequence set forth in SEQ ID NO: 110, and a light chain variable region comprising the amino acid sequence set forth in SEQ ID NO: 107.

In other embodiments, the invention features a CAR comprising a heavy chain CDR3 domain comprising the amino acid sequence set forth in SEQ ID NO: 97, a heavy chain CDR2 domain comprising the amino acid sequence set forth in SEQ ID NO: 92, and a heavy chain CDR1 domain comprising the amino acid sequence set forth in SEQ ID NO: 79; and a light chain CDR3 domain comprising the amino acid sequence set forth in SEQ ID NO: 95, a light chain CDR2 domain comprising the amino acid sequence set forth in SEQ ID NO: 45, and a light chain CDR1 domain comprising the amino acid sequence set forth in SEQ ID NO: 83. In other embodiments, the invention features a CAR comprising a heavy chain variable region comprising the amino acid sequence set forth in SEQ ID NO: 115, and a light chain variable region comprising the amino acid sequence set forth in SEQ ID NO: 112.

In other embodiments, the invention features a CAR comprising a heavy chain CDR3 domain comprising the amino acid sequence set forth in SEQ ID NO: 97, a heavy chain CDR2 domain comprising the amino acid sequence set forth in SEQ ID NO: 104, and a heavy chain CDR1 domain comprising the amino acid sequence set forth in SEQ ID NO: 79; and a light chain CDR3 domain comprising the amino acid sequence set forth in SEQ ID NO: 102, a light chain CDR2 domain comprising the amino acid sequence set forth in SEQ ID NO: 45, and a light chain CDR1 domain comprising the amino acid sequence set forth in SEQ ID NO: 83. In other embodiments, the invention features a CAR comprising a heavy chain variable region comprising the amino acid sequence set forth in SEQ ID NO: 118, and a light chain variable region comprising the amino acid sequence set forth in SEQ ID NO: 117.

In some embodiments, the invention provides an anti-CD98 Antibody Drug Conjugate (ADC) comprising an anti-CD98 antibody of the invention, e.g., huAb102, huAb104, huAb105, huAb110, conjugated to a drug via a linker. In some embodiments, the drug is an auristatin or a pyrrolobenzodiazepine (PBD). In some embodiments, the drug is a Bcl-xL inhibitor. In some embodiments, the linker is a cleavable linker. In some embodiments, the linker is a non-cleavable linker. In some embodiments, the linker is maleimidocaproyl, valine-citrulline, p-aminobenzylalcohol (mc-vc-PABA).

In some embodiments, the anti-human CD98 (hCD98) antibody drug conjugate (ADC) comprises a drug linked to an anti-human CD98 (hCD98) antibody by way of a linker, wherein the drug is a Bcl-xL inhibitor according to structural formula (IIa) or (IIb):

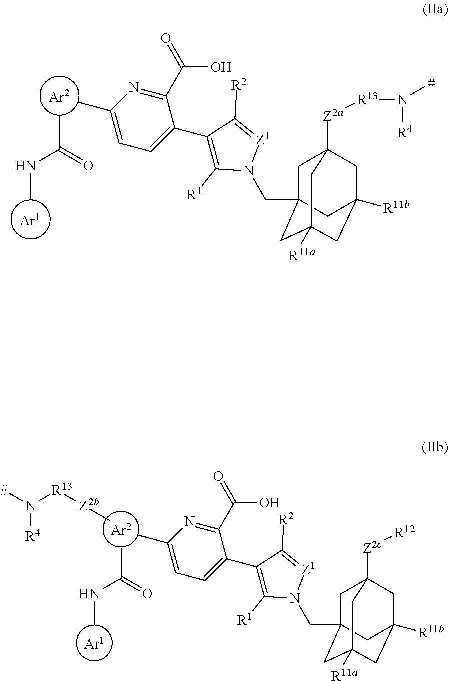

wherein:

Ar¹ is selected from

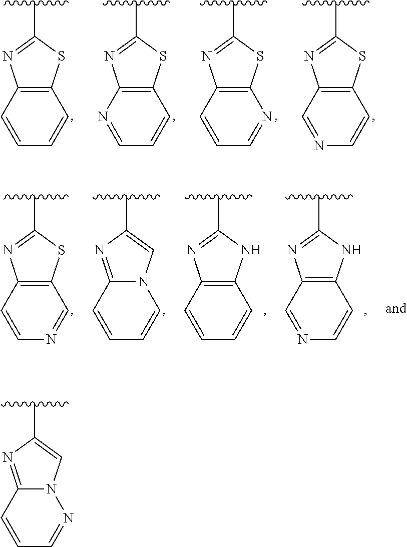

and is optionally substituted with one or more substituents independently selected from halo, hydroxy, nitro, lower alkyl, lower heteroalkyl, C₁₋₄alkoxy, amino, cyano and halomethyl;

Ar² is selected from

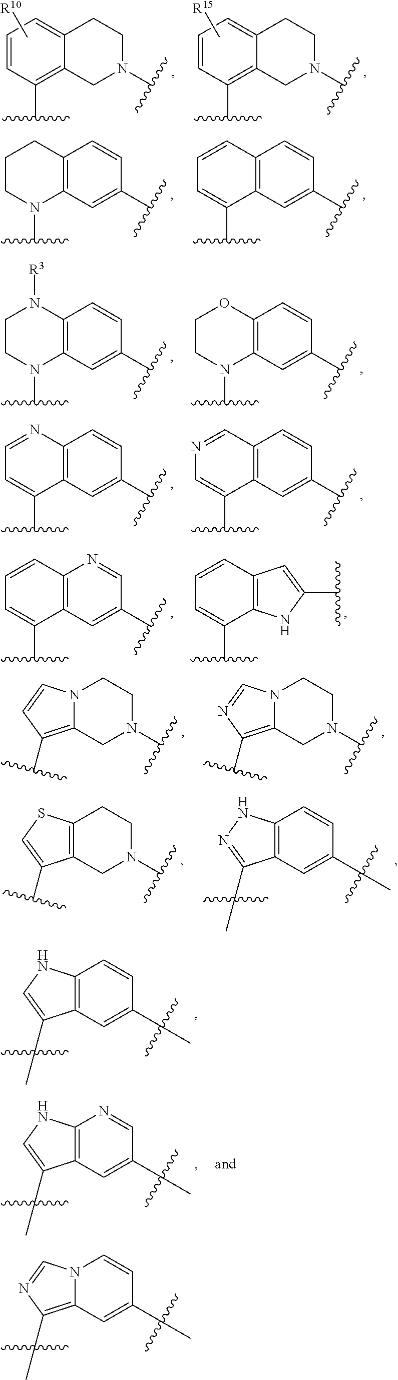

and is optionally substituted with one or more substituents independently selected from halo, hydroxy, nitro, lower alkyl, lower heteroalkyl, C₁₋₄alkoxy, amino, cyano and halomethyl, wherein the #—N(R⁴)—R¹³—Z^{2b}— substituent of formula (IIb) is attached to Ar² at any Ar² atom capable of being substituted;

Z¹ is selected from N, CH, C-halo and C—CN;

Z^{2a}, Z^{2b}, and Z^{2c} are each, independent from one another, selected from a bond, NR⁶, CR^{6a}R^{6b}, O, S, S(O), SO₂, NR⁶C(O), NR^{6a}C(O)NR^{6b}, and NR⁶C(O)O;

R¹ is selected from hydrogen, methyl, halo, halomethyl, ethyl and cyano;

R² is selected from hydrogen, methyl, halo, halomethyl and cyano;

R³ is selected from hydrogen, lower alkyl and lower heteroalkyl; R⁴ is selected from hydrogen, lower alkyl, monocyclic cycloalkyl, monocyclic heterocyclyl, and lower heteroalkyl or is taken together with an atom of R¹³ to form a cycloalkyl or heterocyclyl ring having between 3 and 7 ring atoms, wherein the lower alkyl, monocyclic cycloalkyl, monocyclic heterocyclyl, and lower heteroalkyl are optionally substituted with one or more halo, cyano, hydroxy, C₁₋₄alkoxy, monocyclic cycloalkyl, monocyclic heterocyclyl, C(O)NR^{6a}R^{6b}, S(O)₂NR^{6a}R^{6b}, NHC(O)CHR^{6a}R^{6b}, NHS(O)CHR^{6a}R^{6b}, NHS(O)₂CHR^{6a}R^{6b}, S(O)₂CHR^{6a}R^{6b} or S(O)₂NH₂ groups;

R⁶, R^{6a} and R^{6b} are each, independent from one another, selected from hydrogen,

lower alkyl, lower heteroalkyl, optionally substituted monocyclic cycloalkyl and monocyclic heterocyclyl, or are taken together with an atom from R¹³ to form a cycloalkyl or heterocyclyl ring having between 3 and 7 ring atoms;

R¹⁰ is selected from cyano, OR¹⁴, SR¹⁴, SOR¹⁴, SO₂R¹⁴, SO₂NR^{14a}R^{14b}, NR^{14a}R^{14b}, NHC(O)R¹⁴ and NHSO₂R¹⁴;

R^{11a} and R^{11b} are each, independently of one another, selected from hydrogen, halo, methyl, ethyl, halomethyl, hydroxyl, methoxy, CN, and SCH₃;

R¹² is selected from hydrogen, halo, cyano, lower alkyl, lower heteroalkyl, cycloalkyl, and heterocyclyl, wherein the alkyl, heteroalkyl, cycloalkyl, and heterocyclyl are optionally substituted with one or more halo, cyano, C₁₋₄alkoxy, monocyclic cycloalkyl, monocyclic heterocyclyl, NHC(O)CHR^{6a}R^{6b}, NHS(O)CHR^{6a}R^{6b}, NHS(O)CHR^{6a}R^{6b} or S(O)₂CHR^{6a}R^{6b} groups;

R¹³ is selected from a bond, optionally substituted lower alkylene, optionally substituted lower heteroalkyl tone, optionally substituted cycloalkyl or optionally substituted heterocyclyl;

R¹⁴ is selected from hydrogen, optionally substituted lower alkyl and optionally-substituted lower heteroalkyl;

R^{14a} and R^{14b} are each, independently of one another, selected from hydrogen, optionally substituted lower alkyl, and optionally substituted lower heteroalkyl, or are taken together with the nitrogen atom to which they are bonded to form an optionally substituted monocyclic cycloalkyl or monocyclic heterocyclyl ring;

R¹⁵ is selected from hydrogen, halo, C₁₋₆ alkanyl, C₂₋₄ alkenyl, C₂₋₄ alkynyl, and C₁₋₄ haloalkyl and C₁₋₄ hydroxyalkyl, with the proviso that when R¹⁵ is present, R⁴ is not C₁₋₄ alkyl, C₂₋₄ alkenyl, C₂₋₄ alkynyl, C₁₋₄ haloalkyl or C₁₋₄ hydroxyalkyl, wherein the R⁴ C₁₋₆ alkanyl, C₂₋₄ alkenyl, C₂₋₄ alkynyl, C₁₋₄ haloalkyl and C₁₋₄ hydroxyalkyl are optionally substituted with one or more substituents independently selected from OCH₃, OCH₂CH₂OCH₃, and OCH₂CH₂NHCH₃; and
# represents a point of attachment to a linker; and

wherein the anti-hCD98 antibody is selected from the group consisting of

an anti-hCD98 antibody comprising a heavy chain CDR3 domain comprising the amino acid sequence set forth in SEQ ID NO: 17, a heavy chain CDR2 domain comprising the amino acid sequence set forth in SEQ ID NO: 87, and a heavy chain CDR1 domain comprising the amino acid sequence set forth in SEQ ID NO: 16; a light chain CDR3 domain comprising the amino acid sequence set forth in SEQ ID NO: 19, a light chain CDR2 domain comprising the amino acid sequence set forth in SEQ ID NO: 7, and a light chain CDR1 domain comprising the amino acid sequence set forth in SEQ ID NO: 13;

an anti-hCD98 antibody comprising a heavy chain CDR3 domain comprising the amino acid sequence set forth in SEQ ID NO: 17, a heavy chain CDR2 domain comprising the amino acid sequence set forth in SEQ ID NO: 90, and a heavy chain CDR1 domain comprising the amino acid sequence set forth in SEQ ID NO: 16; a light chain CDR3 domain comprising the amino acid sequence set forth in SEQ ID NO: 19, a light chain CDR2 domain comprising the amino acid sequence set forth in SEQ ID NO: 7, and a light chain CDR1 domain comprising the amino acid sequence set forth in SEQ ID NO: 13;

an anti-hCD98 antibody comprising a heavy chain CDR3 domain comprising the amino acid sequence set forth in SEQ ID NO: 97, a heavy chain CDR2 domain comprising the amino acid sequence set forth in SEQ ID NO: 92, and a heavy chain CDR1 domain comprising the amino acid sequence set forth in SEQ ID NO: 79; a light chain CDR3 domain comprising the amino acid sequence set forth in SEQ ID NO: 95, a light chain CDR2 domain comprising the amino acid sequence set forth in SEQ ID NO: 45, and a light chain CDR1 domain comprising the amino acid sequence set forth in SEQ ID NO: 83; and

an anti-hCD98 antibody comprising a heavy chain CDR3 domain comprising the amino acid sequence set forth in SEQ ID NO: 97, a heavy chain CDR2 domain comprising the amino acid sequence set forth in SEQ ID NO: 104, and a heavy chain CDR1 domain comprising the amino acid sequence set forth in SEQ ID NO: 79; a light chain CDR3 domain comprising the amino acid sequence set forth in SEQ ID NO: 102, a light chain CDR2 domain comprising the amino acid sequence set forth in SEQ ID NO: 45, and a light chain CDR1 domain comprising the amino acid sequence set forth in SEQ ID NO: 83.

In some embodiments, the ADC comprises a compound according to structural formula (I):

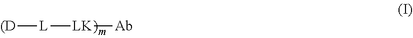

wherein:
D is the Bcl-xL inhibitor drag of formula (IIa) or (IIb);
L is the linker;
Ab is the anti-hCD98 antibody;
LK represents a covalent linkage linking the linker (L) to the anti-hCD98 antibody (Ab); and
m is an integer ranging from 1 to 20.

In some embodiments, Ar¹ is unsubstituted.

In some embodiments, Ar¹ is

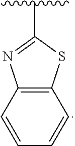 [SMILES: *c1nc2ccccc2s1]

In some embodiments, Ar² is unsubstituted.

In some embodiments, Ar² is

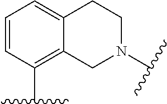 [SMILES: *c1cccc2c1CN(*)CC2]

which is optionally substituted at the 5-position with a group selected from hydroxyl, CM alkoxy, and cyano; or

Ar² is

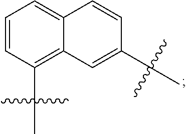 [SMILES: *c1ccc2cccc(*)c2c1]

or

Ar² is

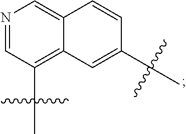 [SMILES: *c1ccc2cncc(*)c2c1]

or

Ar² is

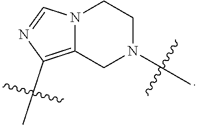 [SMILES: *c1ncn2c1CN(*)CC2]

In some embodiments, Z¹ is N. In some embodiments. Z^{2a} is O. In some embodiments, R¹ is methyl or chloro. In some embodiments, R² is hydrogen or methyl. In some embodiments, R² is hydrogen. In some embodiments, R⁴ is hydrogen or lower alkyl, wherein the lower alkyl is optionally substituted with CM alkoxy or C(O)NR^{6a}R^{6b}.

In some embodiments,

Z¹ is N, Z^{2a} is O, R¹ is methyl or chloro, R² is hydrogen, and Ar² is

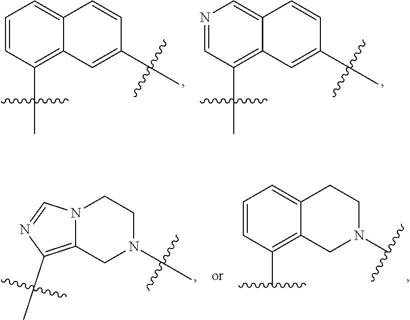 [SMILES: *c1ccc2cccc(*)c2c1 ; *c1ccc2cncc(*)c2c1 ; *c1ncn2c1CN(*)CC2 ; *c1cccc2c1CN(*)CC2]

wherein the

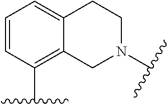 [SMILES: *c1cccc2c1CN(*)CC2]

is optionally substituted at the 5-position with a group selected from hydroxyl. C₁₋₄ alkoxy, and cyano.

In some embodiments, the drug is a Bcl-xL inhibitor according to structural formula (IIa).

In some embodiments, drug is a Bcl-xL inhibitor according to structural formula (IIa).

In some embodiments, Z^{2a} is CH₂ or O.

In some embodiments, R¹³ is selected from lower alkylene or lower heteroalkylene

In some embodiments, the group

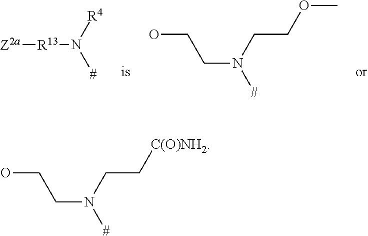

In some embodiments, the group

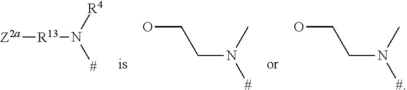

In some embodiments, the group

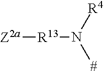

is selected from

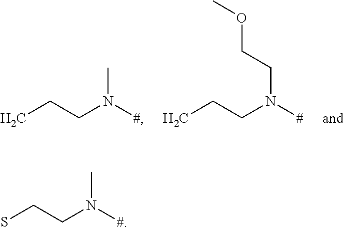

In some embodiments, the group

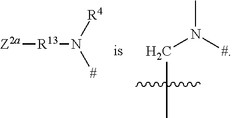

In some embodiments, Z^{2a} oxygen, R¹³ is CH₂CH₂, R⁴ is hydrogen or lower alkyl optionally substituted with C₁₋₄ alkoxy or C(O)NR^{6a}R^{6b}.

In some embodiments, the ADC comprises a compound according to structural formula (IIb).

In some embodiments. Z^{a} is a bond, O, or NR⁶, or and R¹³ is ethylene or optionally substituted heterocyclyl.

In some embodiments, Z^{2c} is O and R¹² is lower alkyl optionally substituted with one or more halo or C₁₋₄alkoxy

In some embodiments, the Bcl-xL inhibitor is selected from the group consisting of the following compounds modified in that the hydrogen corresponding to the # position of structural formula (IIa) or (IIb) is not present forming a monoradical:
6-[1-(1,3-benzothiazol-2-ylcarbamoyl)-1,2,3,4-tetrahydroquinolin-7-yl]-3-[1-({3,5-dimethyl-7-[2-<methylamino)ethoxy]tricyclo[3.3.1.1^{3,7}]dec-1-yl}methyl)-5-methyl-1H-pyrazol-4-yl]pyridine-2-carboxylic acid:
6-[4-(1,3-benzothiazol-2-ylcarbamoyl)-3,4-dihydro-2H-1,4-benzoxazin-6-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
6-[4-(1,3-benzothiazol-2-ylcarbamoyl)-1-methyl-1,2,3,4-tetrahydroquinoxalin-6-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
3-(1-{[3-(2-aminoethoxy)-5,7-dimethyltricyclo[3.3.1.1^{3,7}]dec-1-yl]methyl}-5-methyl-1H-pyrazol-4-yl)-6-[1-(1,3-benzothiazol-2-ylcarbamoyl)-5,6-dihydroimidazo[1,5-a]pyrazin-7(8H)-yl]pyridine-2-carboxylic acid;
3-(1-{[3-(2-aminoethoxy)-5,7-dimethyltricyclo[3.3.1.1^{3,7}]dec-1-yl]methyl}-5-methyl-1H-pyrazol-4-yl)-6-[8-(1,3-benzothiazol-2-ylcarbamoyl)-5-hydroxy-3,4-dihydroisoquinolin-2(1H)-yl]pyridine-2-carboxylic acid;
6-[8-(1,3-benzothiazol-2-ylcarbamoyl)naphthalen-2-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
3-[1-({3,5-dimethyl-7-[2-(methylamino)ethoxy]tricyclo[3.3.1.1^{3,7}]dec-1-yl}methyl)-5-methyl-1H-pyrazol-4-yl]-6-[8-([1,3]thiazolo[4,5-b]pyridin-2-ylcarbamoyl)naphthalen-2-yl]pyridine-2-carboxylic acid;
3-[1-({3,5-dimethyl-7-[2-(methylamino)ethoxy]tricyclo[3.3.1.1^{3,7}]dec-1-yl}methyl)-5-methyl-1H-pyrazol-4-yl]-6-[8-([1,3]thiazolo[5,4-b]pyridin-2-ylcarbamoyl)naphthalen-2-yl]pyridine-2-carboxylic acid;
6-[8-(1,3-benzothiazol-2-ylcarbamoyl)-5-methoxy-3,4-dihydroisoquinolin-2(1H)-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
6-[5-(1,3-benzothiazol-2-ylcarbamoyl)quinolin-3-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid:
6-[4-(1,3-benzothiazol-2-ylcarbamoyl)quinolin-6-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
6-[8-(1,3-benzothiazol-2-ylcarbamoyl)-5-methoxy-3,4-dihydroisoquinolin-2(1H)-yl]-3-{1-[(3-{2-[(2-methoxyethyl)amino]ethoxy}-5,7-dimethyltricyclo[3.3.1.1^{3,7}]dec-1-yl)methyl]-5-methyl-1H-pyrazol-4-yl}pyridine-2-carboxylic acid;
3-(1-{[3-(2-aminoethoxy)-5,7-dimethyltricyclo[3.3.1.13,7]dec-1-yl]methyl}-5-methyl-1H-pyrazol-4-yl)-6-[8-(1,3-benzothiazol-2-ylcarbamoyl)-5-cyano-3,4-dihydroisoquinolin-2(1H)-yl]pyridine-2-carboxylic acid;
6-[1-(1,3-benzothiazol-2-ylcarbamoyl)-1,2,3,4-tetrahydroquinolin-7-yl]-3-{1-[(3-{2-[(2-methoxyethyl)amino]ethoxy}-5,7-dimethyltricyclo[3.3.1.1^{3,7}]dec-1-yl)methyl]-5-methyl-1H-pyrazol-4-yl}pyridine-2-carboxylic acid;
6-[8-(1,3-benzothiazol-2-ylcarbamoyl)naphthalen-2-yl]-3-{1-[(3-{2-[(2-methoxyethyl)amino]ethoxy}-5,7-dimethyltricyclo[3.3.1.1^{3,7}]dec-1-yl)methyl]-5-methyl-1H-pyrazol-4-yl}pyridine-2-carboxylic acid;
6-[8-(1,3-benzothiazol-2-ylcarbamoyl)-3,4-dihydroisoquinolin-2(1H)-yl]-3-[1-({3,5-dimethyl-7-[2-(oxetan-3-ylamino)ethoxy]tricyclo[3.3.1.1^{3,7}]dec-1-yl}methyl)-5-methyl-1H-pyrazol-4-yl]pyridine-2-carboxylic acid;
6-[6-(3-aminopyrrolidin-1-yl)-8-(1,3-benzothiazol-2-ylcarbamoyl)-3,4-dihydroisoquinolin-2(1H)-yl]-3-(1-{[3-(2-methoxyethoxy)-5,7-dimethyltricyclo[3.3.1.1^{3,7}]dec-1-yl]methyl}-5-methyl-1H-pyrazol-4-yl)pyridine-2-carboxylic acid;
6-[8-(1,3-benzothiazol-2-ylcarbamoyl)-3,4-dihydroisoquinolin-2(1H)-yl]-3-{1-[(3,5-dimethyl-7-{2-[(2-sulfamoylethyl)amino]ethoxy}tricyclo[3.3.1.1^{3,7}]dec-1-yl)methyl]-5-methyl-1H-pyrazol-4-yl}pyridine-2-carboxylic acid;
3-(1-{[3-(2-aminoethoxy)-5,7-dimethyltricyclo[3.3.1.1^{3,7}]dec-1-yl]methyl}-5-methyl-1H-pyrazol-4-yl)-6-[3-(1,3-benzothiazol-2-ylcarbamoyl)-6,7-dihydrothieno[3,2-c]pyridin-5(4H)-yl]pyridine-2-carboxylic acid;
3-(1-{[3-(2-aminoethoxy)-5,7-dimethyltricyclo[3.3.1.1^{3,7}]dec-1-yl]methyl}-5-methyl-1H-pyrazol-4-yl)-6-[1-(1,3-benzothiazol-2-ylcarbamoyl)-3-(trifluoromethyl)-5,6-dihydroimidazo[1,5-a]pyrazin-7(8H)-yl]pyridine-2-carboxylic acid;
6-[8-(1,3-benzothiazol-2-ylcarbamoyl)-6-{methyl[2-(methylamino)ethyl]amino}-3,4-dihydroisoquinolin-2(1H)-yl]-3-(1-{[3-(2-methoxyethoxy)-5,7-dimethyltricyclo[3.3.1.1^{3,7}]dec-1-yl]methyl}-5-methyl-1H-pyrazol-4-yl)pyridine-2-carboxylic acid;
6-[8-(1,3-benzothiazol-2-ylcarbamoyl)-6-methoxy-3,4-dihydroisoquinolin-2(1H)-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
3-(1-{[3-(2-aminoethoxy)-5,7-dimethyltricyclo[3.3.1.1^{3,7}]dec-1-yl]methyl}-5-methyl-1H-pyrazol-4-yl)-6-[4-(1,3-benzothiazol-2-ylcarbamoyl)quinolin-6-yl]pyridine-2-carboxylic acid;
6-[5-amino-8-(1,3-benzothiazol-2-ylcarbamoyl)-3,4-dihydroisoquinolin-2(1H)-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
6-[8-(1,3-benzothiazol-2-ylcarbamoyl)-6-[3-(methylamino)prop-1-yn-1-yl]-3,4-dihydroisoquinolin-2(1H)-yl]-3-(1-{[3-(2-methoxyethoxy)-5,7-dimethyltricyclo[3.3.1.1^{3,7}]dec-1-yl]methyl}-5-methyl-1H-pyrazol-4-yl)pyridine-2-carboxylic acid;
6-[4-(1,3-benzothiazol-2-ylcarbamoyl)isoquinolin-6-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
6-[7-(1,3-benzothiazol-2-ylcarbamoyl)-1H-indol-2-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
3-(1-{[3-(2-aminoethoxy)-5,7-dimethyltricyclo[3.3.1.1^{3,7}]dec-1-yl]methyl}-5-methyl-1H-pyrazol-4-yl)-6-[7-(1,3-benzothiazol-2-ylcarbamoyl)-1H-indol-2-yl]pyridine-2-carboxylic acid;
6-[7-(1,3-benzothiazol-2-ylcarbamoyl)-3-methyl-1H-indol-2-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
6-[8-(1,3-benzothiazol-2-ylcarbamoyl)-3,4-dihydroisoquinolin-2(1H)-yl]-3-(1-{[3,5-dimethyl-7-(2-{[l-(methylsulfonyl)piperidin-4-yl]amino}ethoxy)tricyclo[3.3.1.1^{3,7}]dec-1-yl]methyl}-5-methyl-1H-pyrazol-4-yl)pyridine-2-carboxylic acid;
6-[8-(1,3-benzothiazol)-2-ylcarbamoyl)-3,4-dihydroisoquinolin-2(1H)-yl]-3-(1-{[3,5-dimethyl-7-(2-{[1-(methylsulfonyl)azetidin-3-yl]amino}ethoxy)tricyclo[3.3.1.1^{3,7}]dec-1-yl]methyl}-5-methyl-1H-pyrazol-4-yl)pyridine-2-carboxylic acid;
3-{1-[(3-{2-[(3-amino-3-oxopropyl)amino]ethoxy}-5,7-dimethyltricyclo[3.3.1.1^{3,7}]dec-1-yl)methyl]-5-methyl-1H-pyrazol-4-yl}-6-[8-(1,3-benzothiazol-2-ylcarbamoyl)-3,4-dihydroisoquinolin-2(1H)-yl]pyridine-2-carboxylic acid;
6-[3-(1,3-benzothiazol-2-ylcarbamoyl)-1H-indazol-5-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
6-[3-(1,3-benzothiazol-2-ylcarbamoyl)-1H-indol-5-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
6-[3-(1,3-benzothiazol-2-ylcarbamoyl)-1H-pyrrolo[2,3-b]pyridin-5-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
6-(8-(benzo[d]thiazol-2-ylcarbamoyl)-3,4-dihydroisoquinolin-2(1H)-yl)-3-(1-((3-(2-((2-(N,N-dimethylsulfamoyl)ethyl)amino)ethoxy)-5,7-dimethyladamantan-1-yl)methyl)-5-methyl-1H-pyrazol-4-yl)picolinic acid:
6-[8-(1,3-benzothiazol-2-ylcarbamoyl)naphthalen-2-yl]-3-{1-[(3-{2-[(3-hydroxypropyl)amino]ethoxy}-5,7-dimethyltricyclo[3.3.1.1^{3,7}]dec-1-yl)methyl]-5-methyl-1H-pyrazol-4-yl}pyridine-2-carboxylic acid;
6-[8-(1,3-benzothiazol-2-ylcarbamoyl)-3,4-dihydroisoquinolin-2(1H)-yl]-3-(1-{[3-(2-{[3-(dimethylamino)-3-oxopropyl]amino}ethoxy)-5,7-dimethyltricyclo[3.3.1.1^{3,7}]dec-1-yl]methyl}-5-methyl-1H-pyrazol-4-yl)pyridine-2-carboxylic acid;
6-[8-(1,3-benzothiazol-2-ylcarbamoyl)-3,4-dihydroisoquinolin-2(1H)-yl]-3-(1-{[3,5-dimethyl-7-(2-{[3-(methylamino)-3-oxopropyl]amino}ethoxy)tricyclo[3.3.1.1^{3,7}]dec-1-yl]methyl}-5-methyl-1H-pyrazol-4-yl)pyridine-2-carboxylic acid;
3-(1-{[3-(2-aminoacetamido)-5,7-dimethyltricyclo[3.3.1.1^{3,7}]decan-1-yl]methyl}-5-methyl-1H-pyrazol-4-yl)-6-{8-[(1,3-benzothiazol-2-yl)carbamoyl]-3,4-dihydroisoquinolin-2(1H)-yl}pyridine-2-carboxylic acid;
3-[1-({3-[(2-aminoethyl)sulfanyl]-5,7-dimethyltricyclo[3.3.1.1^{3,7}]dec-1-yl}methyl)-5-methyl-1H-pyrazol-4-yl]-6-[8-(1,3-benzothiazol-2-ylcarbamoyl)-3,4-dihydroisoquinolin-2(1H)-yl]pyridine-2-carboxylic acid;
3-(1-{[3-(3-aminopropyl)-5,7-dimethyltricyclo[3.3.1.1^{3,7}]dec-1-yl]methyl}-5-methyl-1H-pyrazol-4-yl)-6-[8-(1,3-benzothiazol-2-ylcarbamoyl)-3,4-dihydroisoquinolin-2(1H)-yl]pyridine-2-carboxylic acid; and
3-(1-{[3-(2-aminoethoxy)-5,7-dimethyltricyclo[3.3.1.1^{3,7}]decan-1-yl]methyl}-5-methyl-1H-pyrazol-4-yl)-6-{5-[(1,3-benzothiazol-2-yl)carbamoyl]quinolin-3-yl}pyridine-2-carboxylic acid.

In some embodiments, the linker is cleavable by a lysosomal enzyme. In some embodiments, the lysosomal enzyme is Cathepsin B.

In some embodiments, the linker comprises a segment according to structural formula (IVa), (IVb), (IVc), or (IVd):

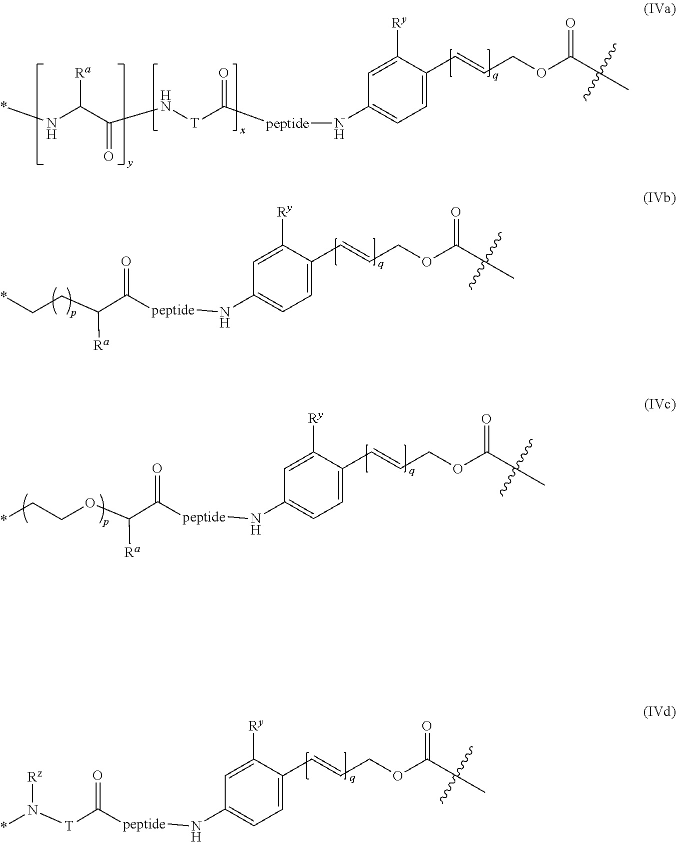

wherein:
peptide represents a peptide (illustrated N→C, wherein peptide includes the amino and carboxy “termini”) a cleavable by a lysosomal enzyme;
T represents a poly mer comprising one or more ethylene glycol units or an alkylene chain, or combinations thereof;
R^{a} is selected from hydrogen, C₁₋₆ alkyl, SO₃H and CH₂SO₃H;
R^{y} is hydrogen or C₁₋₄ alkyl-(O)ᵣ—(C₁₋₄ alkylene)ₛ-G¹ or C₁₋₄ alkyl-(N)—[(C₁₋₄ alkylene)-G¹]₂;
R^{z} is C₁₋₄ alkyl-(O)ᵣ—(C₁₋₄ alkylene)ₛ-G²;
G¹ is SO₃H, CO₂H. PEG 4-32, or sugar moiety;
G² is SO₃H, CO₂H, or PEG 4-32 moiety;
r is 0 or 1;
s is 0 or 1;
p is an integer ranging from 0 to 5;
q is 0 or 1;
x is 0 or 1;
y 0 or 1;
[symbol] represents the point of attachment of the linker to the Bcl-xL inhibitor, and
* represents the point of attachment to the remainder of the linker.

In some embodiments, the peptide is selected from the group consisting of Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val: Val-Ala: Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit.

In some embodiments, the lysosomal enzyme is β-glucuronidase or β-galactosidase.

In some embodiments, the linker comprises a segment according to structural formula (Va), (Vb), (Vc), (Vd), or (Ve):

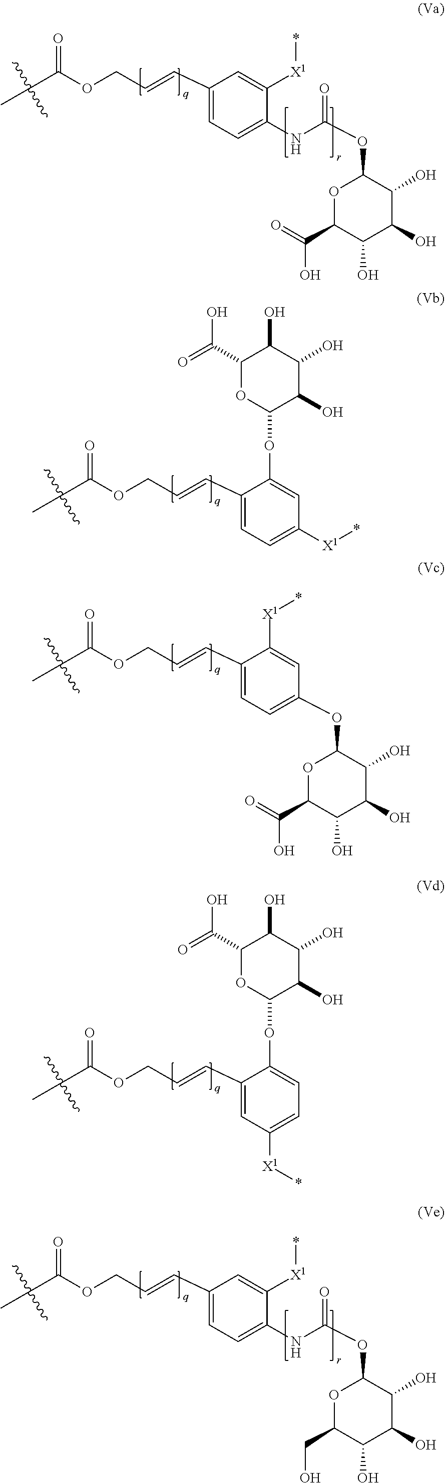

wherein:
q is 0 or 1;
r is 0 or 1;
X¹ is CH₂, O or NH;
[symbol] represents the point of attachment of the linker to the drug; and
* represents the point of attachment to the remainder of the linker.

In some embodiments, the linker comprises a segment according to structural formulae (VIIIa), (VIIIb), or (VIIIc):

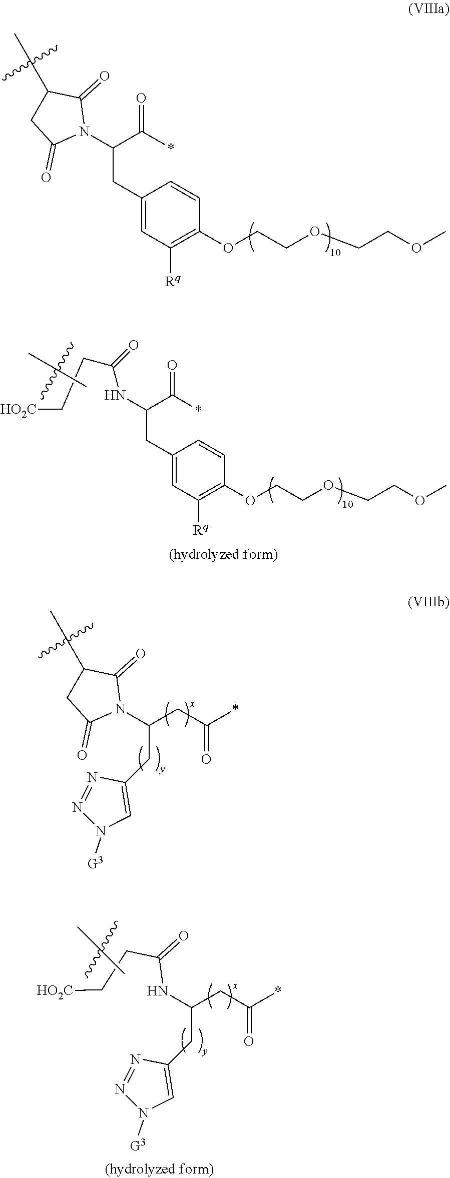

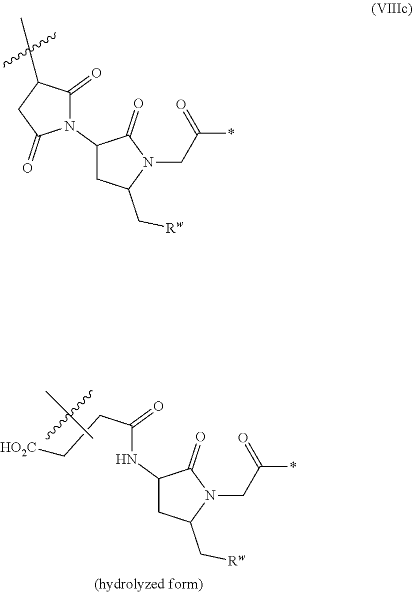

or a hydrolyzed derivative thereof, wherein:

R^{q} is H or —O—(CH₂CH₂O)₁₁—CH₃;

x is 0 or 1;

y is 0 or 1:

G³ is —CH₂CH₂CH₂SO₃H or —CH₂CH₂O—(CH₂CH₂O)₁₁—CH₃;

R^{w} is —O—CH₂CH₂SO₃H or —NH(CO)—CH₂CH₂O(CH₂CH₂O)₁₂—CH₃;

* represents the point of attachment to the remainder of the linker, and

[symbol] represents the point of attachment of the linker to the antibody, wherein when in the hydrolyzed form, [symbol] can be either at the α-position or β-position of the carboxylic acid next to it.

In some embodiments, the linker comprises a polyethylene glycol segment having from 1 to 6 ethylene glycol units.

In some embodiments, m is 2, 3 or 4.

In some embodiments, linker L comprises a segment according to structural formula IVa or IVb.

In some embodiments, linker L is selected from the group consisting of IVa.1-IVa.8. IVb.1-IVb.19, IVc.1-IVc.7, IVd.1-IVd.4, Va.1-Va.12, Vb.1-Vb.10, Vc.1-Vc.11, Vd.1-Vd.6, Ve.1-Ve.2, VIa.1, VIc.1-VIc.2, VId.1-VId.4, VIIa.1-VIIa.4, VIIb.1-VIIb.8, VIIc.1-VIIc.6 in either the closed or open form.

In some embodiments, linker L is selected from the group consisting of IVb.2, IVc.5, IVc.6, IVc.7, IVd.4. Vb.9. Vc.11. VIIa.1, VIIa.3. VIIc.1, VIIc.4, and VIIc.5, wherein the maleimide of each linker has reacted with the antibody Ab, forming a covalent attachment as either a succinimide (closed form) or succinamide (open form).

In some embodiments, linker L is selected from the group consisting of IVb.2, IVc.5, IVc.6, IVd.4, Vc.11, VIIa.1, VIIa.3, VIIc.1, VIIc.4, VIIc.5, wherein the maleimide of each linker has reacted with the antibody Ab, forming a covalent attachment as either a succinimide (closed form) or succinamide (open form).

In some embodiments, linker L is selected from the group consisting of IVb.2, Vc.11, VIIa.3, IVc.6, and VIIc.1, wherein [symbol] is the attachment point to drug D and @ is the attachment point to the LK, wherein when the linker is in the open form as shown below, @ can be either at the α-position or β-position of the carboxylic acid next to it:

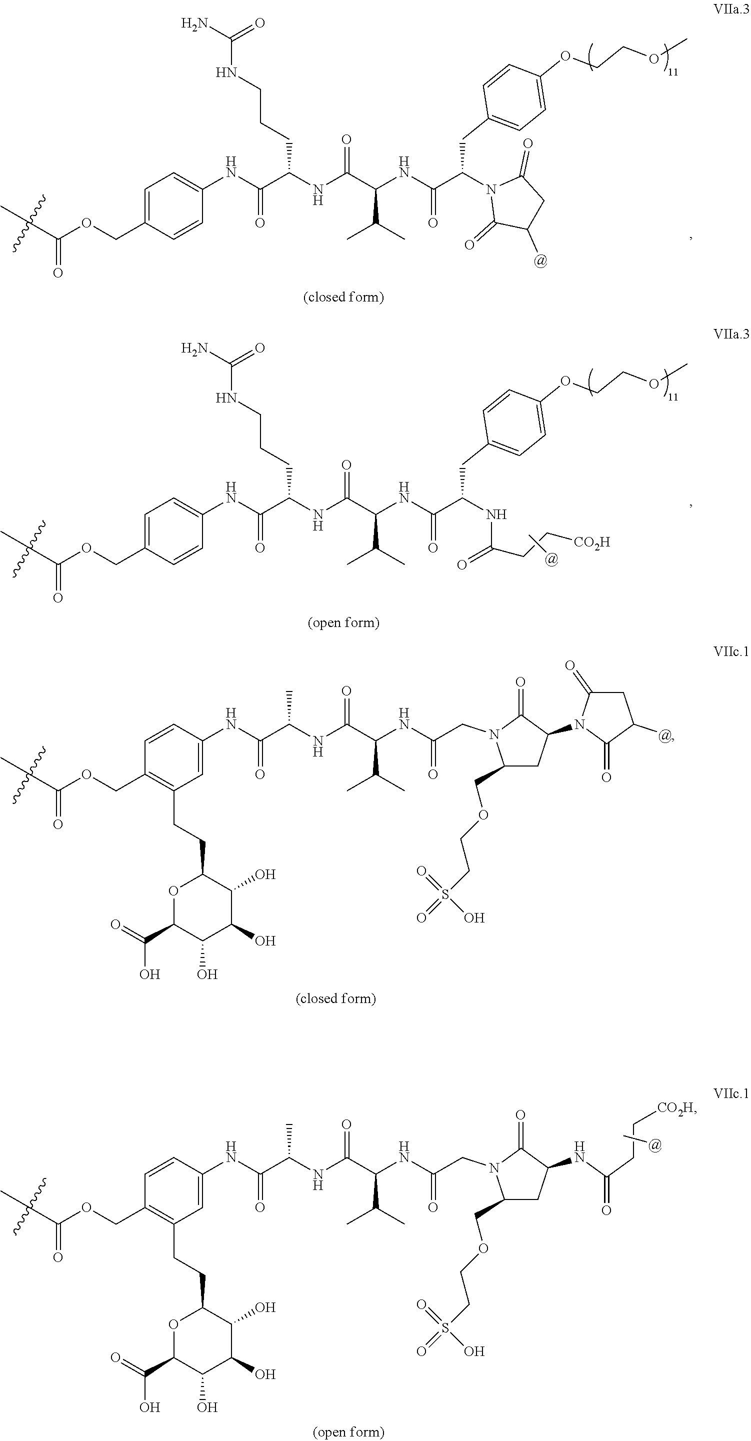

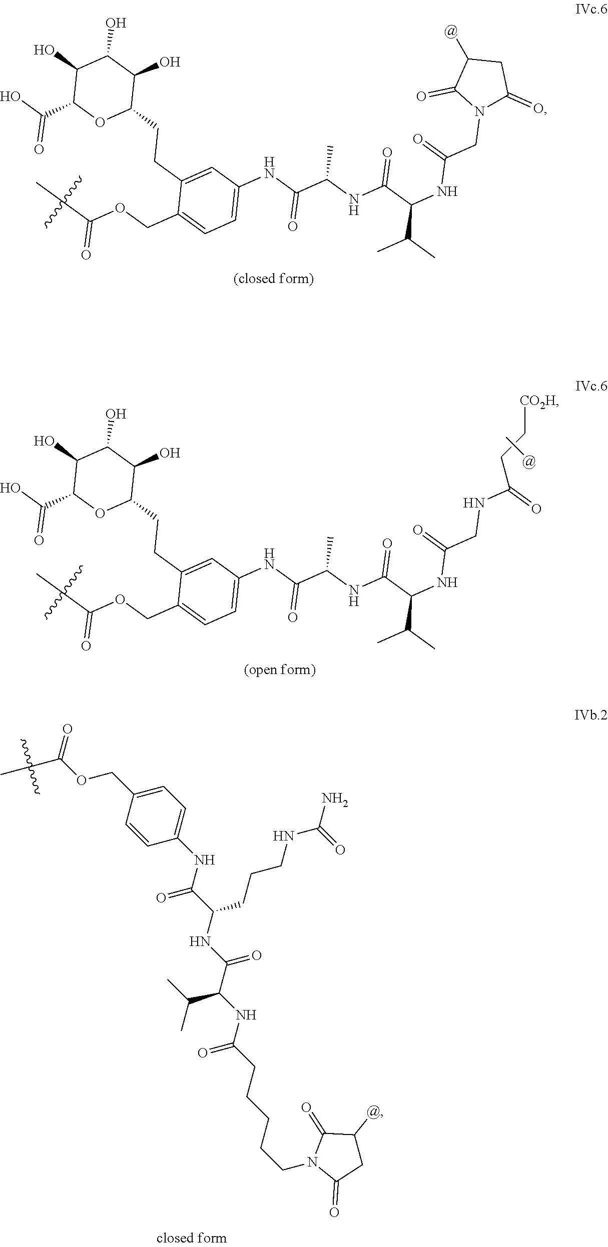

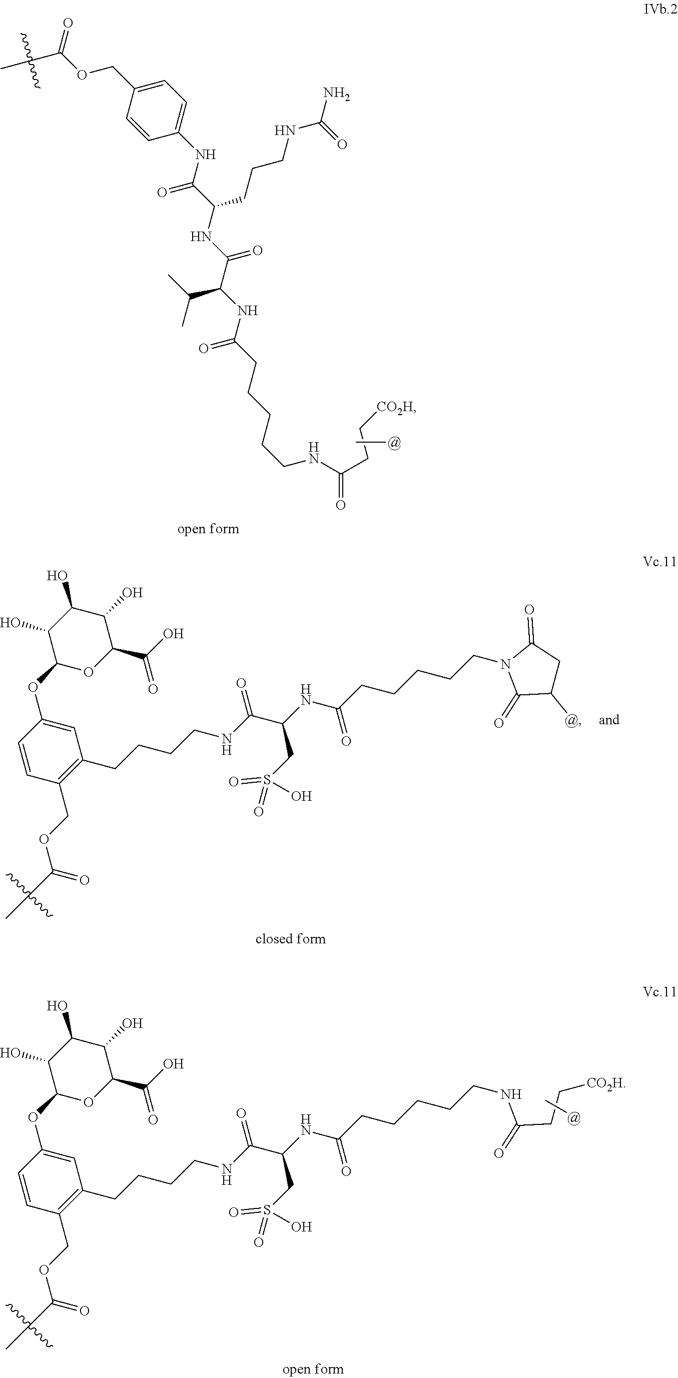

In some embodiments, LK is a linkage formed with an amino group on the anti-hCD98 antibody. In some embodiments, LK is an amide or a thiourea. In some embodiments, LK is a linkage formed with a sulfhydryl group on the anti-hCD98 antibody. In some embodiments, LK is a thioether.

In some embodiments. LK is selected from the group consisting of amide, thiourea and thioether; and m is an integer ranging from 1 to 8.

In some embodiments. D is a Bcl-xL inhibitor as defined herein; L is selected from the group consisting of linkers IVa.1-IVa.8, IVb.1-IVb.19, IVc.1-IVc.7, IVd.1-IVd.4, Va.1-Va.12, Vb.1-Vb.10, Vc.1-Vc.11, Vd.1-Vd.6, Ve.1-Ve.2, VIa.1.VIc.1-VIc.2, VId.1-VId.4. VIIa.1-VIIa.4, VIIb.1-VIIb.8, and VIIc.1-VIIc.6, wherein each linker has reacted with the antibody, Ab, forming a covalent attachment; LK is thioether; and m is an integer ranging from 1 to 8.

In some embodiments, D is the Bcl-xL inhibitor selected from the group consisting of the following compounds modified in that the hydrogen corresponding to the # position of structural formula (IIa) or (IIb) is not present, forming a monoradical:
3-(1-{[3-(2-aminoethoxy)-5,7-dimethyltricyclo[3.3.1.1^{3,7}]dec-1-yl]methyl}-5-methyl-1H-pyrazol-4-yl)-6-[1-(1,3-benzothiazol-2-ylcarbamoyl)-5,6-dihydroimidazo[1,5-a]pyrazin-7(8H)-yl]pyridine-2-carboxylic acid;
6-[8-(1,3-benzothiazol-2-ylcarbamoyl)naphthalen-2-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
6-[8-(1,3-benzothiazol-2-ylcarbamoyl)-5-methoxy-3,4-dihydroisoquinolin-2(1H)-yl]-3-{1-[(3-{2-[(2-methoxyethyl)amino]ethoxy}-5,7-dimethyltricyclo[3.3.1.1^{3,7}]dec-1-yl)methyl]-5-methyl-1H-pyrazol-4-yl} pyridine-2-carboxylic acid;
3-(1-{[3-(2-aminoethoxy)-5,7-dimethyltricyclo[3.3.1.1^{3,7}]dec-1-yl]methyl}-5-methyl-1H-pyrazol-4-yl)-6-[8-(1,3-benzothiazol-2-ylcarbamoyl)-5-cyano-3,4-dihydroisoquinolin-2(1H)-yl]pyridine-2-carboxylic acid;
6-[4-(1,3-benzothiazol-2-ylcarbamoyl)isoquinolin-6-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid; and
3-{1-[(3-{2-[(3-amino-3-oxopropyl)amino]ethoxy}-5,7-dimethyltricyclo[3.3.1.1^{3,7}]dec-1-yl)methyl]-5-methyl-1H-pyrazol-4-yl}-6-[8-(1,3-benzothiazol-2-ylcarbamoyl)-3,4-dihydroisoquinolin-2(1H)-yl]pyridine-2-carboxylic acid;
   L is selected from the group consisting of linkers IVb.2. IVc.5, IVc.6, IVc.7, IVd.4, Vb.9, Vc.11. VIIa.1, VIIa.3, VIIc.1, VIIc.4, and VIIc.5 in either closed or open forms;
   LK is thioether; and
   m is an integer ranging from 2 to 4.

In some embodiments, the ADC is selected from the group consisting of huAb102-ZT, huAb102-ZZ, huAb102-XW, huAb102-SE, huAb102-SR, huAb102-YG, huAb102-KZ, huAb104-ZT, huAb104-ZZ, huAb104-XW, huAb104-SE, huAb104-SR, huAb104-YG, huAb104-KZ, huAb108-ZT, huAb108-ZZ, huAb108-XW, huAb108SE, huAb108-SR, huAb108-YG, huAb108-KZ, huAb110-ZT, huAb110-ZZ, huAb110-XW, huAb110-SE, huAb110-SR, huAb110-YG, and huAb110-KZ, wherein huAb102, huAb104, huAb108, and huAb110 are the anti-hCD98 antibodies and KZ, SR, SE, XW, YG, ZT and ZZ are synthons disclosed in Table 5, and where in the synthons are either in open or closed form.

In some embodiments, the ADC is selected from the group consisting of formulae i-xiv:

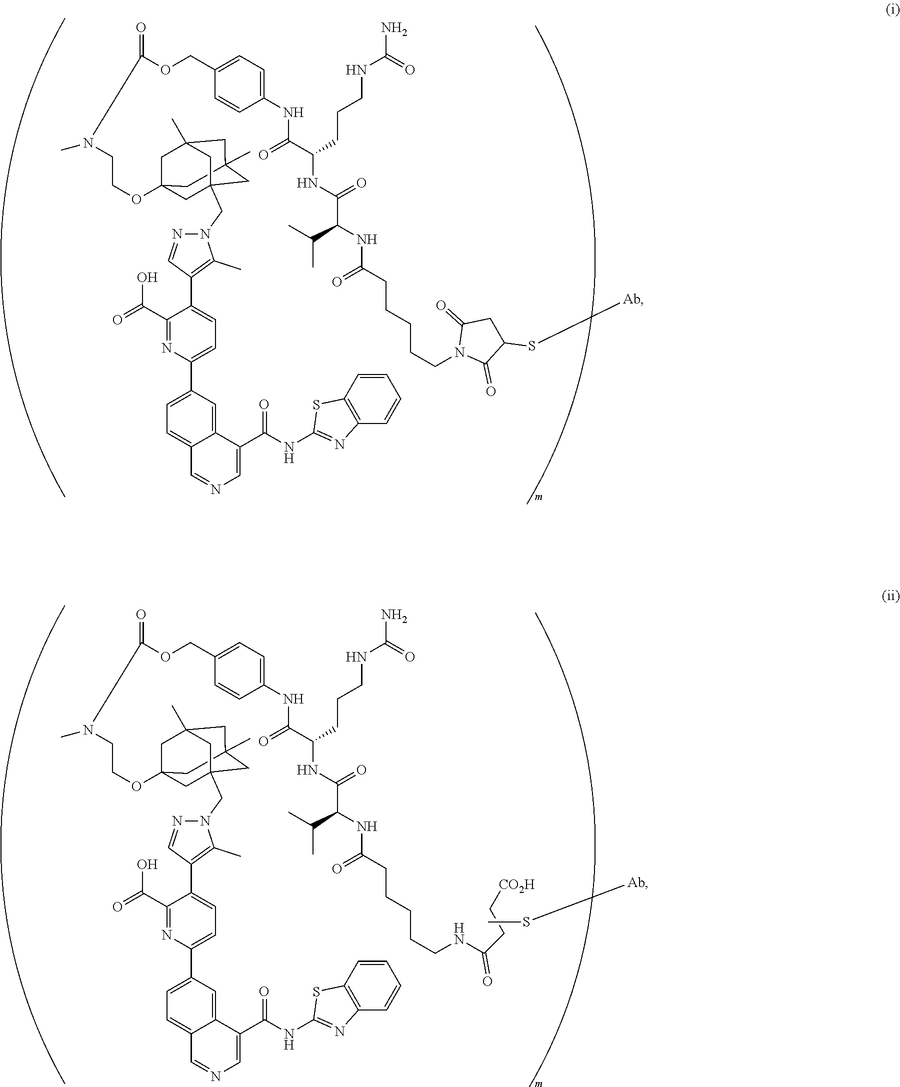

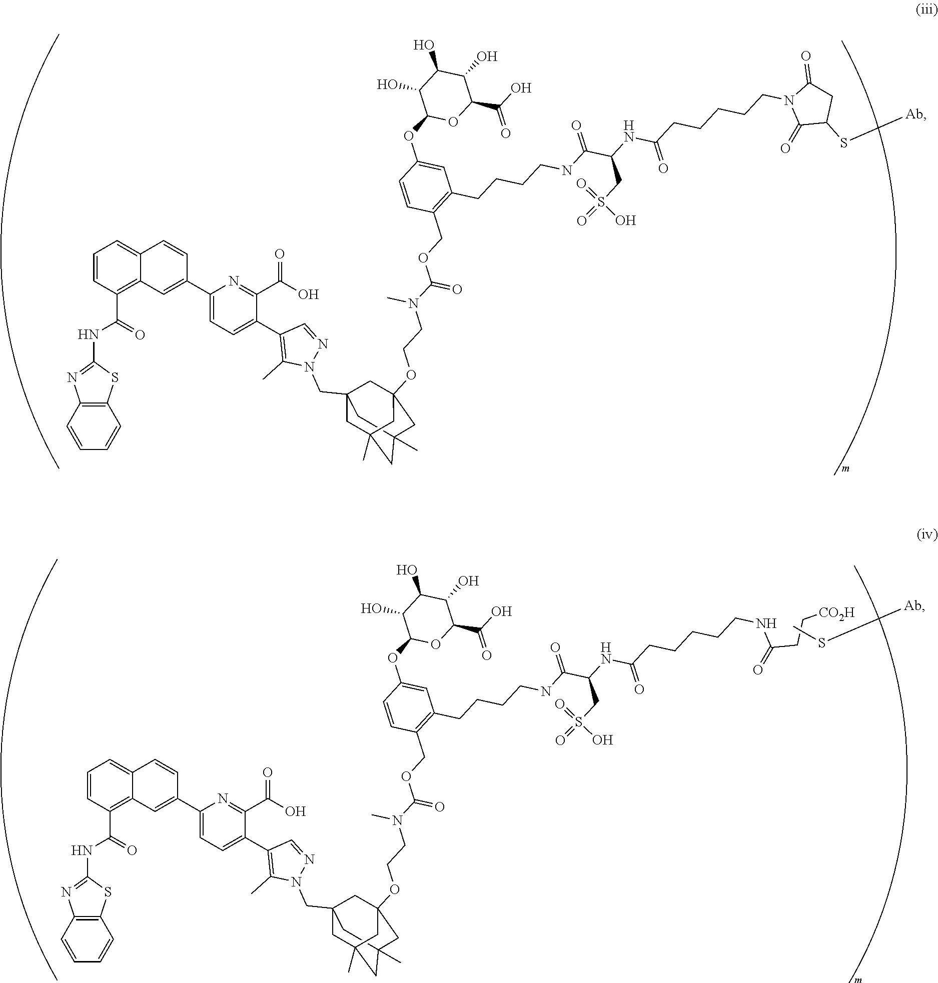

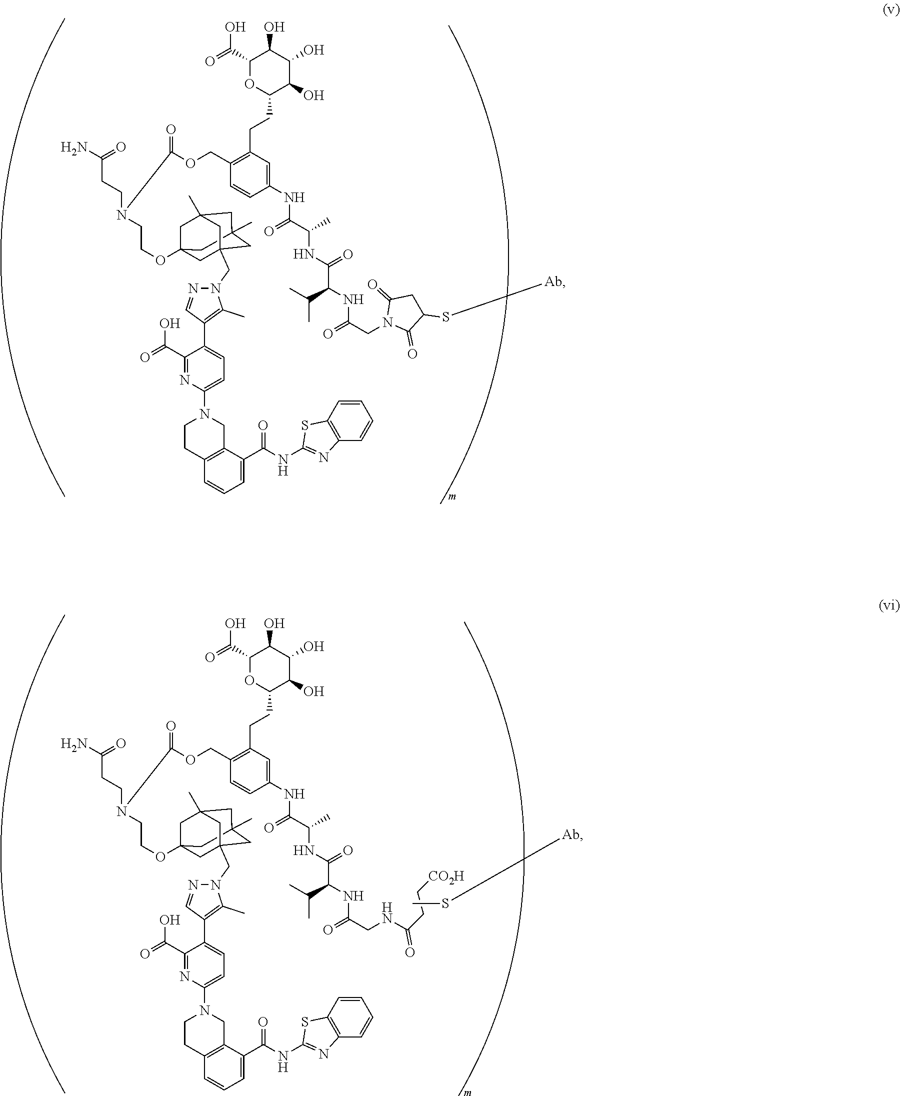

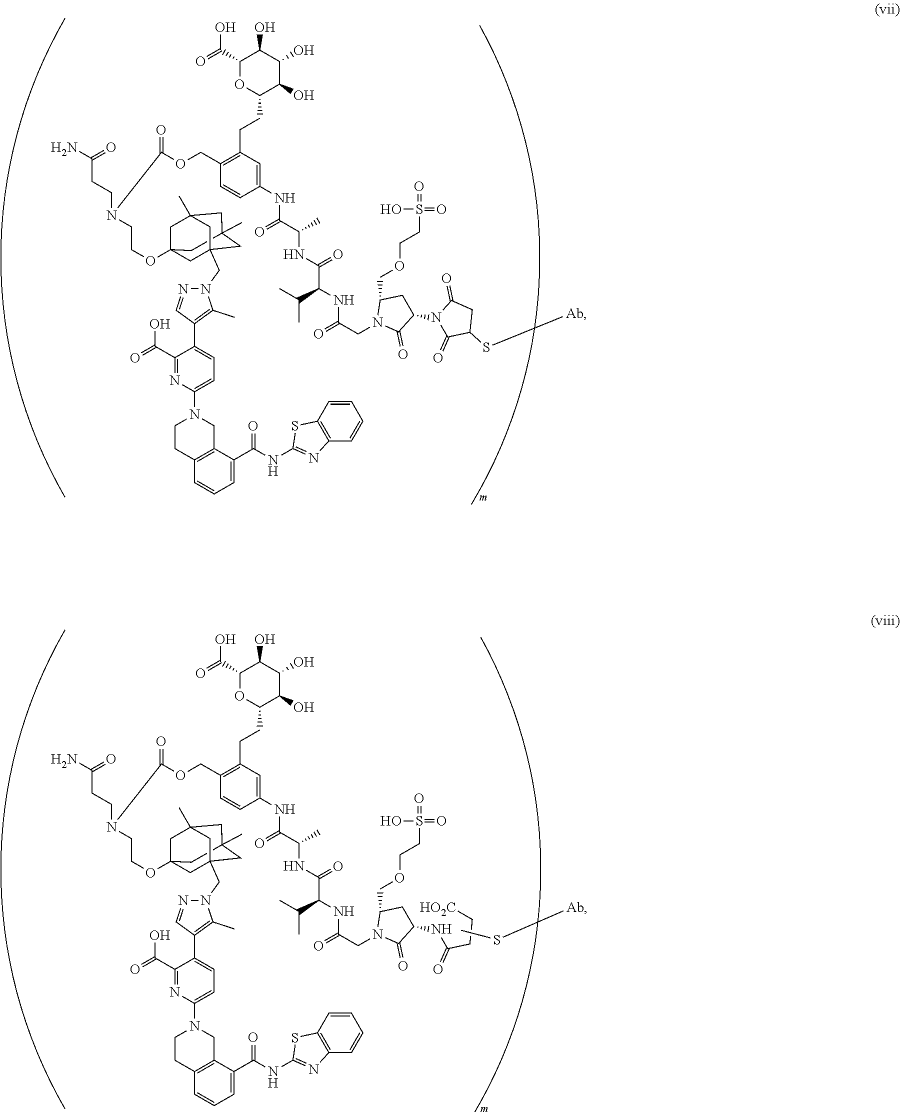

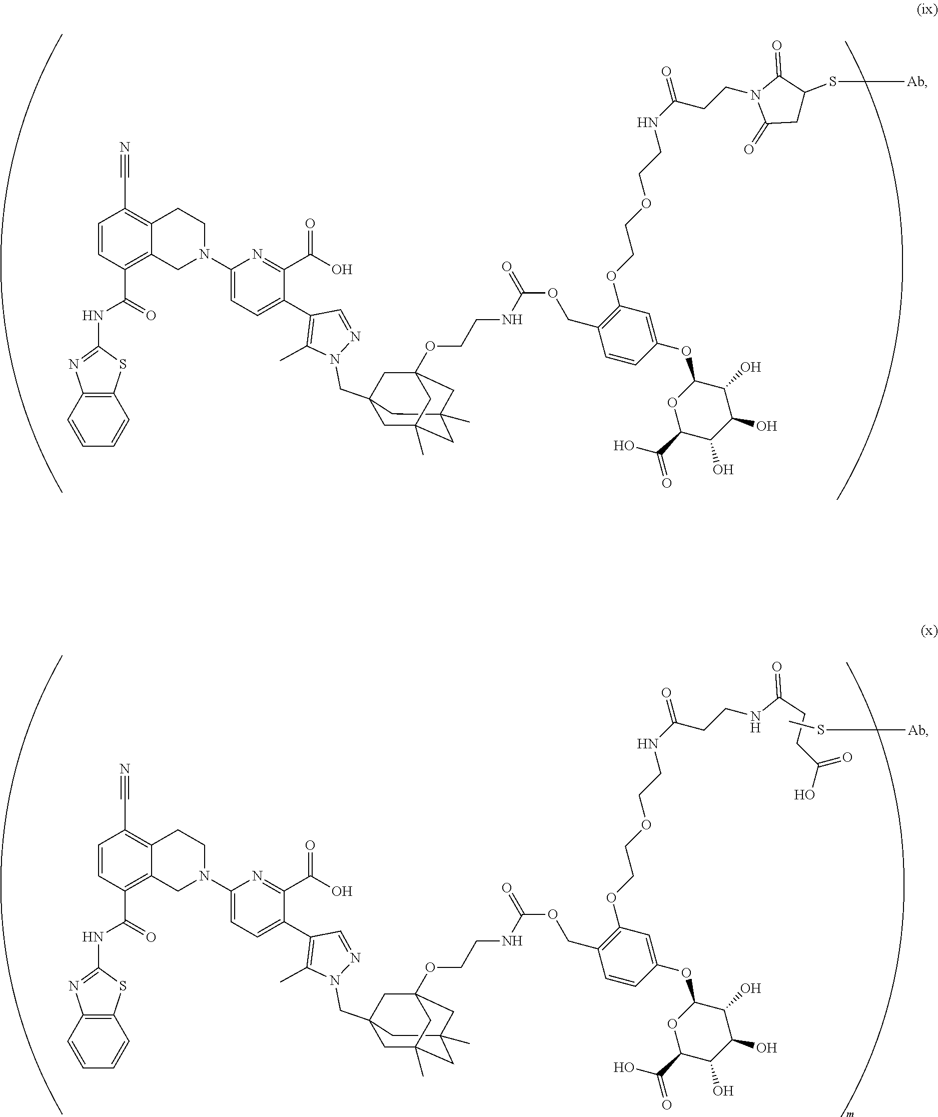

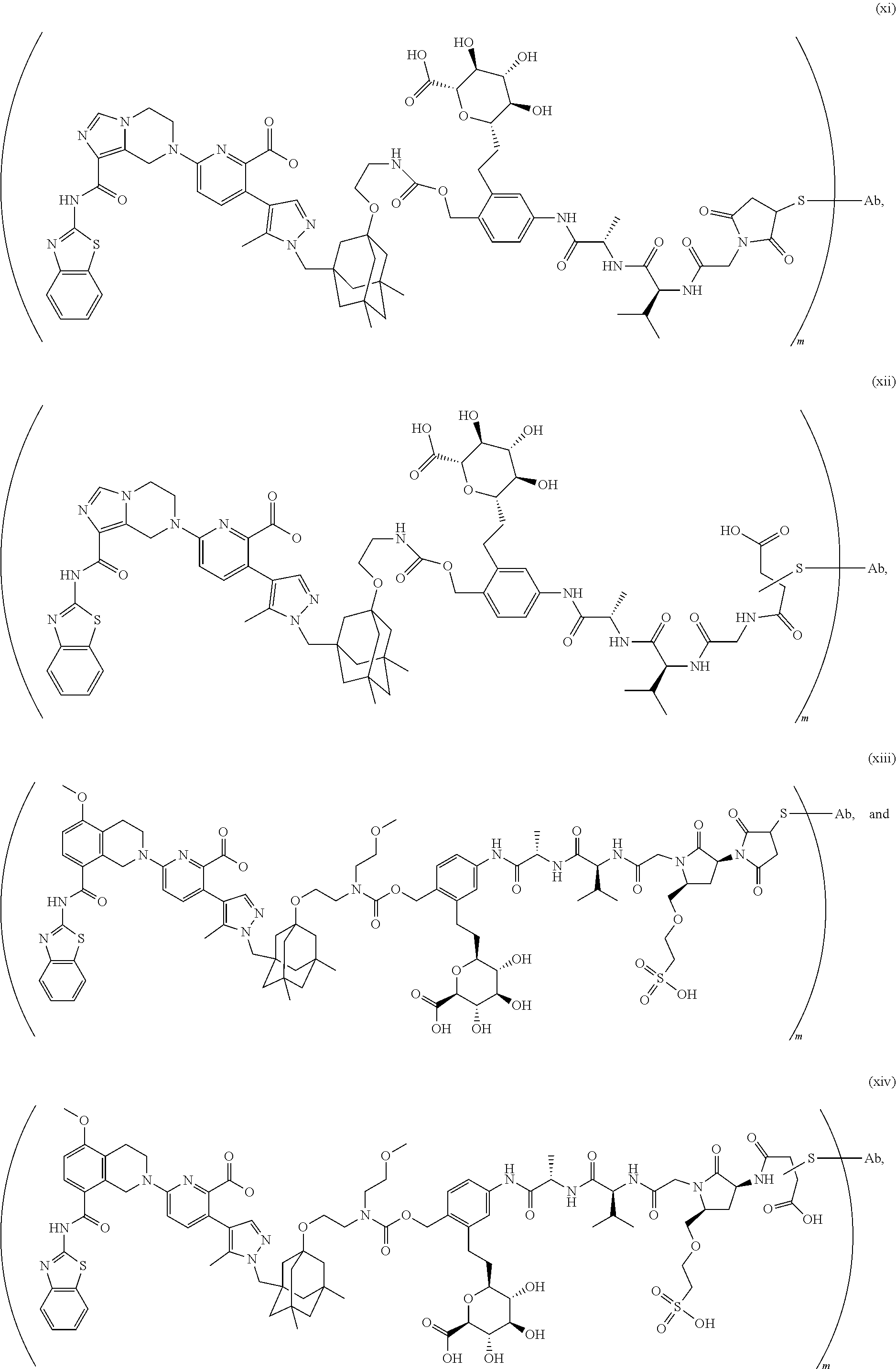

wherein m is an integer from 1 to 6. In a specific embodiment, m is 2. In a specific embodiment, Ab is the anti-hCD98 antibody, wherein the anti-hCD98 antibody comprises the heavy and light chain CDRs of huAb102. In another specific embodiment, Ab is the anti-hCD98 antibody, wherein the anti-hCD98 antibody comprises the heavy and light chain CDRs of huAb104. In a specific embodiment, Ab is the anti-hCD98 antibody, wherein the anti-hCD98 antibody comprises the heavy and light chain CDRs of huAb108. In another specific embodiment, Ab is the anti-hCD98 antibody, wherein the anti-hCD98 antibody comprises the heavy and light chain CDRs of huAb110.

In some embodiments, m is an integer from 2 to 6.

In some embodiments, the antibody comprises a heavy chain variable region comprising the amino acid sequence set forth in SEQ ID NO: 108, and a light chain variable region comprising the amino acid sequence set forth in SEQ ID NO: 107.

In some embodiments, the antibody comprises a heavy chain variable region comprising the amino acid sequence set forth in SEQ ID NO: 110, and a light chain variable region comprising the amino acid sequence set forth in SEQ ID NO: 107.

In some embodiments, the antibody comprises a heavy chain variable region comprising the amino acid sequence set forth in SEQ ID NO: 115, and a light chain variable region comprising the amino acid sequence set forth in SEQ ID NO: 112.

In some embodiments, the antibody comprises a heavy chain variable region comprising the amino acid sequence set forth in SEQ ID NO: 118, and a light chain variable region comprising the amino acid sequence set forth in SEQ ID NO: 117.

In some embodiments, the antibody is a monoclonal IgG1 antibody. In some embodiments, the antibody comprises a kappa light chain.

In some embodiments, the ADC is selected from the group consisting of huAb102-ZT, huAb102-ZZ, huAb102-XW, huAb102-SE, huAb102-SR, huAb102-YG, huAb102-KZ, huAb104-ZT, huAb104-ZZ, huAb104-XW, huAb104-SE, huAb104-SR, huAb104-YG, huAb104-KZ, huAb108-ZT, huAb108-ZZ, huAb108-XW, huAb108-SE, huAb108-SR, huAb108-YG, huAb108-KZ, huAb110-ZT, huAb110-ZZ, huAb110-XW, huAb110-SE, huAb110-SR, huAb110-YG, and huAb110-KZ.

In some embodiments, the invention provides a pharmaceutical composition comprising an effective amount of an ADC of the invention, and a pharmaceutically acceptable carrier. In some embodiments, the invention provides a pharmaceutical composition comprising an ADC mixture comprising a plurality of the ADC of the invention, and a pharmaceutically acceptable carrier.

In some embodiments, the ADC mixture has an average drug to antibody ratio (DAR) of 2 to 4. In some embodiments, the ADC mixture comprises ADCs each having a DAR of 2 to 8.

In some embodiments, the invention provides a method for treating cancer, comprising administering a therapeutically effective amount of the ADC of the invention to a subject in need thereof. In some embodiments, the cancer is selected from the group consisting of small cell lung cancer, non-small cell lung cancer, breast cancer, ovarian cancer, a glioblastoma, prostate cancer, pancreatic cancer, colon cancer, head and neck cancer, multiple myeloma, B cell lymphoma, T cell lymphoma, and acute lymphoblastic leukemia, chronic myeloid leukemia, chronic leukocytic leukemia, Hodgkin lymphoma, acute myeloid leukemia and kidney cancer.

In some embodiments, the cancer is a squamous cell carcinoma. In some embodiments, the squamous cell carcinoma is squamous lung cancer or squamous head and neck cancer. In some embodiments, the cancer is triple negative breast cancer. In some embodiments, the cancer is multiple myeloma. In some embodiments, the cancer is acute myeloid leukemia. In some embodiments, the cancer is non-small cell lung cancer.

In some embodiments, the invention provides a method for inhibiting or decreasing solid tumor growth in a subject having a solid tumor, said method comprising administering an effective amount of the ADC of the invention to the subject having the solid tumor, such that the solid tumor growth is inhibited or decreased.

In some embodiments, the solid tumor is a non-small cell lung carcinoma.

In some embodiments, ADC is administered in combination with an additional agent or an additional therapy. In some embodiments, the additional agent is selected from the group consisting of an anti-PD1 antibody (e.g, pembrolizumab), an anti-PD-L1 antibody (e.g, atezolizumab), an anti-CTLA-4 antibody (e.g, ipilimumab), a MEK inhibitor (e.g, trametinib), an ERK inhibitor, a BRAF inhibitor (e.g, dabrafenib), osimertinib, erlotinib, gefilinib, sorafenib, a CDK9 inhibitor (e.g. dinaciclib), a MCL-1 inhibitor, temozolomide, a Bcl-xL inhibitor, a Bcl-2 inhibitor (e.g. venetoclax), ibrutinib, a mTOR inhibitor (e.g, everolimus), a PI3K inhibitor (e.g, buparlisib), duvelisib, idelalisib, an AKT inhibitor, a HER2 inhibitor (e.g. lapatinib), a taxane (e.g, docetaxel, paclitaxel, nab-paclitaxel), an ADC comprising an auristatin, an ADC comprising a PBD (e.g, rovalpituzumab tesirine), an ADC comprising a maytansinoid (e g. TDM1), a TRAIL agonist, a proteasome inhibitor (e.g, bortezomib), and a nicotinamide phosphoribosyltransferase (NAMPT) inhibitor. In some embodiments, the additional therapy is radiation. In some embodiments, the additional agent is a chemotherapeutic agent.

In some embodiments, the cancer or tumor is characterized as having CD98 overexpression.

In some embodiments, the cancer or tumor is characterized as having an activating EGFR mutation. In some embodiments, the activating EGFR mutation is selected from the group consisting of an exon 19 deletion mutation, a single-point substitution mutation L858R in exon 21, a T790M point mutation, and combinations thereof.

In one aspect, the present invention provides a process for the preparation of an ADC according to structural formula (I):

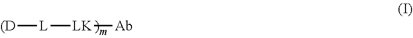

wherein:

D is the Bcl-xL inhibitor drug of formula (IIa) or (IIb) as disclosed herein;

L is the linker as disclosed herein;

Ab is a CD98 antibody, wherein the CD98 antibody comprises the heavy and light chain CDRs of huAb102, huAb014, huAb108, or huAb110;

LK represents a covalent linkage linking linker L to antibody Ab; and

m is an integer ranging from 1 to 20;

the process comprising:

treating an antibody in an aqueous solution with an effective amount of a disulfide reducing agent at 30-40° C. for at least 15 minutes, and then cooling the antibody solution to 20-27° C.;

adding to the reduced antibody solution a solution of water/dimethyl sulfoxide comprising a synthon selected from the group of 2.1 to 2.31 and 2.34 to 2.72 (Table 5);

adjusting the pH of the solution to a pH of 7.5 to 8.5;

allowing the reaction to run for 48 to 80 hours to form the ADC;

wherein the mass is shifted by 18±2 amu for each hydrolysis of a succinimide to a succinamide as measured by electron spray mass spectrometry; and

wherein the ADC is optionally purified by hydrophobic interaction chromatography.

In one embodiment, m is 2.

In another aspect, the present invention provides an ADC prepared by the process of claim 125 or 126.

### BRIEF DESCRIPTION OF THE DRAWINGS

FIG. 1 depicts antibody reduction, modification with a maleimide derivative to give a thiosuccinimide intermediate, and subsequent hydrolysis of thiosuccinimide moiety.

FIG. 2 depicts MS characterization of light chain and heavy chain of huAb108 prior to conjugation, 2) after conjugation to a maleimide derivative to give a thiosuccinimide intermediate and 3) post pH8-mediated hydrolysis of the thiosuccinimide ring.

FIG. 3 provides the structure of an Ab-malemidocaproyl-vc-PABA-MMAEADC (referred to herein as “Ab-vcMMAE”).

FIG. 4 depicts the structure of a PBD dimer (SGD-1882) conjugated to an antibody (Ab) via a maleimidocaproyl-valine-alanine linker (collectively referred to as SGD-1910).

### DETAILED DESCRIPTION OF THE INVENTION

Various aspects of the invention relate to anti-CD98 antibodies and antibody fragments, anti-CD98 ADCs, and pharmaceutical compositions thereof, as well as nucleic acids, recombinant expression vectors and host cells for making such antibodies and fragments. Methods of using the antibodies and ADCs described herein to detect human CD98, to inhibit human CD98 activity (m vitro or in vivo), and to treat cancers such as epithelial cancers, gastric cancer, breast cancer, ovarian cancer, colorectal cancer, head and neck cancers (e.g. glioblastomas), laryngeal cancer, esophageal cancer, lung cancer, kidney cancer, pancreatic cancer, mesothelioma, squamous cell carcinoma (e.g., squamous lung cancer or squamous head and neck cancer), triple negative breast cancer, small cell lung cancer, non-small cell lung cancer, hematological cancers such as multiple myeloma, acute myeloid leukemia, or lymphoma, and prostate cancer are also encompassed by the invention.

An outline of the Detailed Description of the Invention is provided below:

### I. Definitions

### II. Anti-CD98 Antibodies

II. A. Anti-CD98 Chimeric Antibodies

II. B. Humanized Anti-CD98 Antibodies

### III. Anti-CD98 Antibody Drug Conjugates (ADCs)

III. A. Anti-CD98/Bcl-xL Inhibitor ADCs
III.A.1. Bcl-xL Inhibitors
III.A.2. Bcl-xL Linkers
   Cleavable Linkers
   Non-Cleavable Linkers
   Groups Used to Attach Linkers to Anti-CD98 Antibodies
   Linker Selection Considerations
III.A.3. Bcl-xL ADC Synthons
III.A.4. Methods of Synthesis of Bcl-xL ADCs
III.A.5. General Methods for Synthesizing Bcl-xL Inhibitors
III.A.6. General Methods for Synthesizing Anti-CD98 ADCs

III.B. Anti-CD98 ADCs: Other Exemplary Drugs for Conjugation

III.C. Anti-CD98 ADCs: Other Exemplary Linkers

### IV. Purification of Anti-CD98 ADCs

### V. Uses of Anti-CD98 Antibodies and Anti-CD98 ADCs

### VI. Pharmaceutical Compositions

### I. Definitions

In order that the invention may be more readily understood, certain terms are first defined. In addition, it should be noted that whenever a value or range of values of a parameter are recited, it is intended that values and ranges intermediate to the recited values are also intended to be part of this invention.

The terms “anti-CD98 antibody”, as used herein, refers to an antibody that specifically binds to CD98. An antibody “which binds” an antigen of interest. i.e., CD98, is one capable of binding that antigen, e.g., the extracellular domain of CD98, with sufficient affinity such that the antibody is useful in targeting a cell expressing the antigen. In a preferred embodiment, the antibody specifically binds to human CD98 (hCD98), e.g., the extracellular domain of hCD98. Examples of anti-CD98 antibodies are disclosed in the Examples below. Unless otherwise indicated, the term “anti-CD98 antibody” is meant to refer to an antibody which binds to wild type CD98, including the extracellular domain of CD98, or any variant of CD98.

CD98 (also referred to as (also referred to as CD98 heavy chain; 4F2 heavy chain; 4F2hc; SLC3A2) is a type II transmembrane glycoprotein composed of 630 amino acid residues. The protein comprises a 75 amino acid N-terminal intracellular cytoplasmic domain, a single transmembrane domain, and a 425 amino acid C-terminal extracellular domain (Parmacek et al. (1989) Nucleic Acids Res. 17: 1915-1931). An exemplary amino acid sequence of wild-type human CD98 is provided below as SEQ ID NO: 124. The extracellular domain (ECD) of CD98 (SEQ ID NO: 125; underlined), includes amino acids 206-630 of SEQ ID NO: 124.

(SEQ ID NO: 124)
MELQPPEASI AVVSIPRQLP GSHSEAGVQG LSAGDDSELG
SHCVAQTGLE LLASGDPLPS ASQNAEMIET GSDCVTQAGL
QLLASSDPPA LASKNAEVTG TMSQDTEVDM KEVELNELEP
EKQPMNAASG AAMSLAGAEK NGLVKIKVAE DEAEAAAAAK
FTGLSKEELL KVAGSPGWVR TRWALLLLFW LGWLGMLAGA
VVIIVRAPRC RELPAQKWWH TGALYRIGDL QAFQGHGAGN
LAGLKGRLDY LSSLKVKGLV LGPIHKNQKD DVAQTDLLQI
DPNFGSKEDF DSLLQSAKKK SIRVILDLTP NYRGENSWFS
TQVDTVATKV KDALEFWLQA GVDGFQVRDI ENLKDASSFL
AEWQNITKGF SEDRLLIAGT NSSDLQQILS LLESNKDLLL
TSSYLSDSGS TGEHTKSLVT QYLNATGNRW CSWSLSQARL
LTSFLPAQLL RLYQLMLFTL PGTPVFSYGD EIGLDAAALP
GQPMEAPVML WDESSFPDIP GAVSANMTVK GQSEDPGSLL
SLFRRLSDQR SKERSLLHGD FHAFSAGPGL FSYIRHWDQN
ERFLVVLNFG DVGLSAGLQA SDLPASASLP AKADLLLSTQ
PGREEGSPLE LERLKLEPHE GLLLRFPYAA

“Biological activity of CD98” as used herein, refers to all inherent biological properties of the CD98, including, but not limited to, modulation of cell proliferation, survival and/or growth; modulation of integrin signaling; and modulation of amino acid transport.

The terms “specific binding” or “specifically binding”, as used herein, in reference to the interaction of an antibody or an ADC with a second chemical species, mean that the interaction is dependent upon the presence of a particular structure (e.g., an antigenic determinant or epitope) on the chemical species; for example, an antibody recognizes and binds to a specific protein structure rather than to proteins generally. If an antibody or ADC is specific for epitope “A”, the presence of a molecule containing epitope A (or free, unlabeled A), in a reaction containing labeled “A” and the antibody, will reduce the amount of labeled A bound to the antibody or ADC. By way of example, an antibody “binds specifically” to a target (antigen) if the antibody, when labeled, can be competed away from its target by the corresponding non-labeled antibody. In one embodiment, an antibody specifically binds to a target, e.g., CD98, if the antibody has a Ku for the target of at least about 10⁻⁴ M, 10⁻⁵ M, 10−6 M, 10−7 M, 10⁻⁸ M, 10⁻⁹ M, 10⁻¹⁰ M, 10⁻¹¹ M, 10⁻¹² M, or less (less meaning a number that is less than 10⁻¹², e.g. 10⁻¹³). In one embodiment, the term “specific binding to CD98” or “specifically binds to CD98,” as used herein, refers to an antibody or an ADC that binds to CD98 and has a dissociation constant (K_{D}) of 1.0×10⁻⁷ M or less, as determined by surface plasmon resonance. It shall be understood, however, that the antibody or ADC may be capable of specifically binding to two or more antigens which are related in sequence. For example, in one embodiment, an antibody can specifically bind to both human and a non-human (e.g., mouse or non-human primate) orthologs of CD98.

The term “antibody” or “Ab” refers to an immunoglobulin molecule that specifically binds to an antigen and comprises a heavy-(H) chain(s) and a light (L chain(s). Each heavy chain is comprised of a heavy chain variable region (abbreviated herein as HCVR or VH) and a heavy chain constant region. The heavy chain constant region is comprised of three domains. CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. An antibody can be of any type (e.g., IgG, IgE, IgM, IgD, IgA and IgY) and class (e.g., IgG1, IgG2, IgG 3, IgG4, IgA1 and IgA2) or subclass. While the term “antibody” is not intended to include antigen binding portions of an antibody (defined below), it is intended, in certain embodiments, to describe an antibody comprising a small number of amino acid deletions from the carboxy end of the heavy chain(s). Thus, in one embodiment, an antibody comprises a heavy chain having 1-5 amino acid deletions the carboxy-end of the heavy chain. In one embodiment, an antibody is a monoclonal antibody which is an IgG, having four polypeptide chains, two heavy (H) chains, and two light (L chains) that can bind to hEGFR. In one embodiment, an antibody is a monoclonal IgG antibody-comprising a lambda or a kappa light chain.

The term “antigen binding portion” or “antigen binding fragment” of an antibody (or simply “antibody portion” or “antibody fragment”), as used herein, refers to one or more fragments of an antibody that retain the ability to specifically bind to an antigen (e.g., hIL-13). It has been shown that the antigen binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546. Winter et al., PCT publication WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); sec e.g., Bird et at. (1988) Science 242:423-426: and Huston et at. (1988) Proc. Natl. Acad Sci. USA 85:5879-5883). Such single chain antibodies are also intended to be encompassed within the term “antigen binding portion” of an antibody-. In certain embodiments of the invention, scFv molecules may be incorporated into a fusion protein. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger. P., et al. (1993) Proc. Natl. Acad. Sci. USA 90:6444-6448; Poljak. R. J., et al. (1994) Structure 2: 1121-1123). Such antibody binding portions are known in the art (Kontermann and Dubel eds., Antibody Engineering (2001) Springer-Verlag. New York. 790 pp. (ISBN 3-540-41354-5).

An IgG (Immunoglobulin G) is a class of antibody comprising two heavy chains and two light chains arranged in a Y-shape An IgG constant domain refers to a heavy or light chain constant domain. Exemplary human IgG heavy chain and light chain constant domain amino acid sequences are known in the art and represented below.

### Sequence of Human IgG Heavy Chain Constant Domain and Light Chain Constant Domain

| | Sequence | Sequence |
|---|---|---|
| Protein | Identifier | 12345678901234567890123456789012 |
| Ig gamma-1 | SEQ ID NO: 154 | ASTKGPSVFPLAPSSKSTSGGTAALGCLV |
| constant region | | KDYFPEPVTVSWNSGALTSGVHTFPAVLQ |
| | | SSGLYSLSSVVTVPSSSLGTQTYICNVNH |
| | | KPSNTKVDKKVEPKSCDKTHTCPPCPAPE |
| | | LLGGPSVFLFPPKPKDTLMISRTPEVTCV |
| | | VVDVSHEDPEVKFNWYVDGVEVHNAKTKP |
| | | REEQYNSTYRVVSVLTVLHQDWLNGKEYK |
| | | CKVSNKALPAPIEKTISKAKGQPREPQVY |
| | | TLPPSREEMTKNQVSLTCLVKGFYPSDIA |
| | | VEWESNGQPENNYKTTPPVLDSDGSFFLY |
| | | SKLTVDKSRWQQGNVFSCSVMHEALHNHY |
| | | TQKSLSLSPGK |
| Ig gamma-1 | SEQ ID NO: 155 | ASTKGPSVFPLAPSSKSTSGGTAALGCLV |
| constant region | | KDYFPEPVTVSWNSGALTSGVHTFPAVLQ |
| mutant | | SSGLYSLSSVVTVPSSSLGTQTYICNVNH |
| | | KPSNTKVDKKVEPKSCDKTHTCPPCPAPE |
| | | AAGGPSVFLFPPKPKDTLMISRTPEVTCV |
| | | VVDVSHEDPEVKFNWYVDGVEVHNAKTKP |
| | | REEQYNSTYRVVSVLTVLHQDWLNGKEYK |
| | | CKVSNKALPAPIEKTISKAKGQPREPQVY |
| | | TLPPSREEMTKNQVSLTCLVKGFYPSDIA |
| | | VEWESNGQPENNYKTTPPVLDSDGSFFLY |
| | | SKLTVDKSRWQQGNVFSCSVMHEALHNHY |
| | | TQKSLSLSPGK |
| Ig Kappa | SEQ ID NO: 156 | RTVAAPSVFIFPPSDEQLKSGTASVVCLL |
| constant region | | NNFYPREAKVQWKVDNALQSGNSQESVTE |
| | | QDSKDSTYSLSSTLTLSKADYEKHKVYAC |
| | | EVTHQGLSSPVTKSFNRGEC |
| Ig Lambda | SEQ ID NO: 157 | QPKAAPSVTLFPPSSEELQANKATLVCLI |
| constant region | | SDFYPGAVTVAWKADSSPVKAGVETTTPS |
| | | KQSNNKYAASSYLSLTPEQWKSHRSYSCQ |
| | | VTHEGSTVEKTVAPTECS |

An “isolated antibody”, as used herein, is intended to refer to an antibody that is substantially free of other antibodies having different antigenic specificities (e.g., an isolated antibody that specifically binds CD98 is substantially free of antibodies that specifically bind antigens other than CD98). An isolated antibody that specifically binds CD98 may, however, have cross-reactivity to other antigens, such as CD98 molecules from other species. Moreover, an isolated antibody may be substantially free of other cellular material and/or chemicals.

The term “chimeric antibody” refers to antibodies which comprise heavy and light chain variable region sequences from one species and constant region sequences from another species, such as antibodies having murine heavy and light chain variable regions linked to human constant regions.

The term “humanized antibody” refers to antibodies which comprise heavy and light chain variable region sequences from a nonhuman species (e.g., a mouse) but in which at least a portion of the VH and/or VL sequence has been altered to be more “human-like”, i.e., more similar to human germline variable sequences. In particular, the term “humanized antibody” is an antibody or a variant, derivative, analog or fragment thereof which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 80%, preferably at least 85%, at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In other embodiments, a humanized antibody only contains a humanized heavy chain. In specific embodiments, a humanized antibody only contains a humanized variable domain of a light chain and/or humanized heavy chain.

The humanized antibody can be selected from any class of immunoglobulins, including IgM, IgG, IgD, IgA and IgE, and any isotype, including without limitation IgG1, IgG2, IgG3 and IgG4. The humanized antibody may comprise sequences from more than one class or isotype, and particular constant domains may be selected to optimize desired effector functions using techniques well-known in the art.

The terms “Rabat numbering.” “Rabat definitions,” and “Rabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion thereof (Rabat et al. (1971) Ann. NY Acad Sci. 190:382-391 and, Rabat, E. A., et at. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

As used herein, the term “CDR” refers to the complementarity determining region within antibody variable sequences. There are three CDRs in each of the variable regions of the heavy chain (HC) and the light chain (LC), which are designated CDR1, CDR2 and CDR3 (or specifically HC CDR1, HC CDR2, HC CDR3, LC CDR1, LC CDR2, and LC CDR3), for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk. J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined PR's within the variable region of a single, naturally occurring immunoglobulin chain. As used herein, a FR represents one of the four sub-regions, and FRs represents two or more of the four sub-regions constituting a framework region.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In a preferred embodiment, such mutations, however, will not be extensive. Usually, at least 80%, preferably at least 85%, more preferably at least 90% and most preferably at least 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker. From Genes to Clones (Verlagsgesellschaft, Weinheim, Germany 1987). In a family of immunoglobulins, each position in the consensus sequence is occupied by the amino acid occurring most frequently at that position in the family. If two amino acids occur equally frequently, either can be included in the consensus sequence.

The term “human acceptor framework”, as used herein, is meant to refer to a framework of an antibody or antibody fragment thereof comprising the amino acid sequence of a VH or VL framework derived from a human antibody or antibody fragment thereof or a human consensus sequence framework into which CDR's from a non-human species may be incorporated.

“Percent (%) amino acid sequence identity” with respect to a peptide or polypeptide sequence is defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. In one embodiment, the invention includes an amino acid sequence having at least 80%, at least 85%, at least 90%, at least 95%, at least 96%, at least 97%, at least 98% or at least 99% identity to an amino acid sequence set forth in any one of SEQ ID NOs: 1 to 31.35-40, or 50 to 85.

The term “multivalent antibody” is used herein to denote an antibody comprising two or more antigen binding sites. In certain embodiments, the multivalent antibody may be engineered to have the three or more antigen binding sites, and is generally not a naturally occurring antibody.

The term “multispecific antibody” refers to an antibody capable of binding two or more unrelated antigens. In one embodiment, the multispecific antibody is a bispecific antibody that is capable of binding to two unrelated antigens, e.g., a bispecific antibody that binds CD98 and CD3.

The term “dual variable domain” or “DVD,” as used interchangeably herein, are antigen binding proteins that comprise two or more antigen binding sites and are tetravalent or multivalent binding proteins. Such DVDs may be monospecific, i.e., capable of binding one antigen or multispecific, i.e. capable of binding two or more antigens. DVD binding proteins comprising two heavy chain DVD polypeptides and two light chain DVD polypeptides are referred to a DVD Ig. Each half of a DVD Ig comprises a heavy chain DVD polypeptide, and a light chain DVD polypeptide, and two antigen binding sites. Each binding site comprises a heavy chain variable domain and a light chain variable domain with a total of 6 CDRs involved in antigen binding per antigen binding site. In one embodiment, the CDRs described herein are used in an anti-CD98 DVD.

The term “chimeric antigen receptor” or “CAR” refers to a recombinant protein comprising at least (1) an antigen-binding region, e.g., a variable heavy or light chain of an antibody, (2) a transmembrane domain to anchor the CAR into a T cell, and (3) one or more intracellular signaling domains.

The term “activity” includes activities such as the binding specificity/affinity of an antibody or ADC for an antigen, for example, an anti-hCD98 antibody that binds to an hCD98 antigen and/or the neutralizing potency of an antibody, for example, an anti-hCD98 antibody whose binding to hCD98 inhibits the biological activity of hCD98, e.g., modulation of cell proliferation, survival and/or growth; modulation of integrin signaling; and modulation of amino acid transport in an CD98 expressing cell line, e.g., human lung carcinoma cell line A549, human lung carcinoma cell line NCI—H460, non-small cell lung cancer line EBC-1, small cell lung cancer line NCI—H146, non-small cell lung cancer line H2170, breast cancer cell line HCC38, a Molt-4 human acute lymphoblastic leukemia cell line, or a Jurkat acute T cell leukemia cell line.

The term “non small-cell lung carcinoma (NSCLC) xenograft assay.” as used herein, refers to an in vivo assay used to determine whether an anti-CD98 antibody or ADC, can inhibit tumor growth (e.g., further growth) and/or decrease tumor growth resulting from the transplantation of NSCLC cells into an immunodeficient mouse. An NSCLC xenograft assay includes transplantation of NSCLC cells into an immunodeficient mouse such that a tumor grows to a desired size, e.g., 200-250 mm³, whereupon the anti both or ADC is administered to the mouse to determine whether the antibody or ADC can inhibit and/or decrease tumor growth. In certain embodiments, the activity of the antibody or ADC is determined according to the percent tumor growth inhibition (% TGI) relative to a control antibody, e.g., a human IgG antibody (or collection thereof) which does not specifically bind tumor cells, e.g., is directed to an antigen not associated with cancer or is obtained from a source which is noncancerous (e.g., normal human serum). In such embodiments, the antibody (or ADC) and the control antibody are administered to the mouse at the same dose, with the same frequency, and via the same route. In one embodiment, the mouse used in the NSCLC xenograft assay is a severe combined immunodeficiency (SCID) mouse and/or an athymic CD-1 nude mouse. Examples of NSCLC cells that may be used in the NSCLC xenograft assay include, but are not limited to, H2170 cells (e.g., NCI—H2170[H2170] (ATCC® CRL-5928™).

The term “epitope” refers to a region of an antigen that is bound by an antibody or ADC. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. In certain embodiments, an antibody is said to specifically bind an antigen when it preferentially recognizes its target antigen in a complex mixture of proteins and/or macromolecules.

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB. Uppsala, Sweden and Piscataway, N.J.). For further descriptions, see Jönsson, U., et al. (1993) Ann. Biol. Clin. 51:19-26; Jönsson, U., et al (1991) Biotechniques 11:620-627; Johnsson, B., et al. (1995) J. Mol. Recognit. 8:125-131; and Johnnson, B., et al. (1991) Anal. Biochem. 198:268-277. In one embodiment, surface plasmon resonance is determined according to the methods described in Example 4

The term “kₒₙ” or “kₐ”, as used herein, is intended to refer to the on rate constant for association of an antibody to the antigen to form the antibody/antigen complex.

The term “k_{off}” or “k_{d}”, as used herein, is intended to refer to the off rate constant for dissociation of an antibody from the antibody/antigen complex.

The term “K_{D}”, as used herein, is intended to refer to the equilibrium dissociation constant of a particular antibody-antigen interaction (e.g., huAb102, huAb104, huAb108, or huAb110 antibody and CD98). K_{D} is calculated by kₐ/k_{d}.

The term “competitive binding”, as used herein, refers to a situation in which a first antibody competes with a second antibody, for a binding site on a third molecule, e.g., an antigen. In one embodiment, competitive binding between two antibodies is determined using FACS analysis.

The term “competitive binding assay” is an assay used to determine whether two or more antibodies bind to the same epitope. In one embodiment, a competitive binding assay is a competition fluorescent activated cell sorting (FACS) assay which is used to determine whether two or more antibodies bind to the same epitope by determining whether the fluorescent signal of a labeled antibody is reduced due to the introduction of a non-labeled antibody, where competition for the same epitope will lower the level of fluorescence. The term “labeled antibody” as used herein, refers to an antibody, or an antigen binding portion thereof, with a label incorporated that provides for the identification of the binding protein, e.g., an antibody. Preferably, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides (e.g., ³H ¹⁴C, ³⁵S, ⁹⁰Y, ⁹⁹Tc, ¹¹¹In, ¹²⁵I, ¹³¹I, ¹⁷⁷Lu, ¹⁶⁶HO, or ¹⁵³Sm); fluorescent labels (e.g., FITC, rhodamine, lanthanide phosphors), enzymatic labels (e.g., horseradish peroxidase, luciferase, alkaline phosphatase); chemiluminescent markers; biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates.

The term “antibody-drug-conjugate” or “ADC” refers to a binding protein, such as an antibody or antigen binding fragment thereof, chemically linked to one or more chemical drug(s) (also referred to herein as agent(s)) that may optionally be therapeutic or cytotoxic agents. In a preferred embodiment, an ADC includes an antibody, a cytotoxic or therapeutic drug, and a linker that enables attachment or conjugation of the drug to the antibody. An ADC typically has anywhere from 1 to 8 drugs conjugated to the antibody, including drug loaded species of 2, 4, 6, or 8. Non-limiting examples of drugs that may be included in the ADCs are mitotic inhibitors, antitumor antibiotics, immunomodulating agents, vectors for gene therapy, alkylating agents, antiangiogenic agents, antimetabolites, boron-containing agents, chemoprotective agents, hormones, antihormone agents, corticosteroids, photoactive therapeutic agents, oligonucleotides, radionuclide agents, topoisomerase inhibitors, kinase inhibitors, and radiosensitizers. In one embodiment, the drug is a Bcl-xL inhibitor. In a preferred embodiment, the ADC of the invention comprises an anti-CD98 monoclonal IgG antibody conjugated via a linker to a Bcl-xL inhibitor.

The terms “anti-CD98 antibody drug conjugate,” or “anti-CD98 ADC”, used interchangeably herein, refer to an ADC comprising an antibody that specifically binds to CD98, whereby the antibody is conjugated to one or more chemical agent(s). In a preferred embodiment, the anti-CD98 ADC binds to human CD98 (hCD98).

The term “Bcl-xL inhibitor”, as used herein, refers to a compound which antagonizes Bcl-xL activity in a cell. In one embodiment, a Bcl-xL inhibitor promotes apoptosis of a cell by inhibiting Bcl-xL activity.

The term “auristatin”, as used herein, refers to a family of antimitotic agents. Auristatin derivatives are also included within the definition of the term “auristatin”. Examples of auristatins include, but are not limited to, auristatin E (AE), monomethylauristatin E (MMAE), monomethylauristatin F (MMAF), and synthetic analogs of dolastatin. In one embodiment, an anti-CD98 antibody described herein is conjugated to an auristatin to form an anti-CD98 ADC.

As used herein, the term “mcMMAF” is used to refer to a linker/drug combination of maleimidocaproyl-monomethylauristatin F (MMAF).

Various chemical substituents are defined below. In some instances, the number of carbon atoms in a substituent (e.g., alkyl, alkanyl, alkenyl, alkynyl, cycloalkyl, heterocyclyl, heteroaryl, and aryl) is indicated by the prefix “Cₓ-C_{y}” or “C_{x-y}” wherein x is the minimum and y is the maximum number of carbon atoms. Thus, for example, “C₁-C₆ alkyl” refers to an alkyl containing from 1 to 6 carbon atoms. Illustrating further, “C₃-C₈ cycloalkyl” means a saturated hydrocarbon ring containing from 3 to 8 carbon ring atoms. If a substituent is described as being “substituted,” a hydrogen atom on a carbon or nitrogen is replaced with a non-hydrogen group. For example, a substituted alkyl substituent is an alkyl substituent in which at least one hydrogen atom on the alkyl is replaced with a non-hydrogen group. To illustrate, monofluoroalkyl is alkyl substituted with a fluoro radical, and difluoroalkyl is alkyl substituted with two fluoro radicals. It should be recognized that if there is more than one substitution on a substituent, each substitution may be identical or different (unless otherwise stated). If a substituent is described as being “optionally substituted”, the substituent may be either (1) not substituted or (2) substituted. Possible substituents include, but are not limited to, C₁-C₆ alkyl, C₂-C₆, alkenyl, C₂-C₆ alkynyl, aryl, cycloalkyl, heterocyclyl, heteroaryl, halogen, C₁-C₆ haloalkyl, oxo, —CN, NO₂, —OR^{xa}, —OC(O)R^{xz}, —OC(O)N(R^{xa})₂, —SR^{xa}, —S(O)₂R^{xa}, —S(O)₂N(R^{xa})₂, —C(O)R^{xa}, —C(O)OR^{xa}, —C(O)N(R^{xa})₂, —C(O)N(R^{xa})S(O)₂R^{xz}, —N(R^{xa})₂, —N(R^{xa})C(O)R^{xz}, —N(R^{xa})S(O)₂R^{xz}, —N(R^{xa})C(O)O(R^{xz}), —N(R^{xa})C(O)N(R^{xa})₂, —N(R^{xa})S(O)₂N(R^{xa})₂, —(C₁-C₆ alkylenyl)-CN, —(C₁-C₆ alkylenyl)-OR^{xa}, —(C₁-C₆ alkylenyl)-OC(O)R^{xz}, —(C₁-C₆ alkylenyl)-OC(O)N(R^{xa})₂, —(C₁-C₆ alkylenyl)-SR^{xa}, —(C₁-C₆ alkylenyl)-S(O)₂R^{xa}, —(C₁-C₆ alkylenyl)-S(O)₂N(R^{xa})₂, —(C₁-C₆ alkylenyl)-C(O)R^{xa}, —(C₁-C₆ alkylenyl)-C(O)OR^{xa}, —(C₁-C₆ alkylenyl)-C(O)N(R^{xa})₂, —(C₁-C₆ alkylenyl)-C(O)N(R^{xa})S(O)₂R^{xz}, —(C₁-C₆ alkylenyl)-N(R^{xa})₂, —(C₁-C₆ alkylenyl)-N(R^{xa})C(O)R^{xz}, —(C₁-C₆ alkylenyl)-N(R^{xa})S(O)₂R^{xz}, —(C₁-C₆ alkylenyl)-N(R^{xa})C(O)O(R^{xz}), —(C₁-C₆ alkylenyl)-N(R^{xa})C(O)N(R^{xa})₂, or —(C₁-C₆ alkylenyl)-N(R^{xa})S(O)₂N(R^{xa})₂; wherein R^{xa}, at each occurrence, is independently hydrogen, aryl, cycloalkyl, heterocyclyl, heteroaryl, C₁-C₆ alkyl, or C₁-C₆ haloalkyl; and R^{xz}, at each occurrence, is independently aryl, cycloalkyl, heterocyclyl, heteroaryl, C₁-C₆ alkyl or C₁-C₆ haloalkyl.

Various ADCs, synthons and Bcl-xL inhibitors comprising the ADCs and/or synthons are described in some embodiments herein by reference to structural formulae including substituents. It is to be understood that the various groups comprising substituents may be combined as valence and stability permit. Combinations of substituents and variables envisioned by this disclosure are only those that result in the formation of stable compounds. As used herein, the term “stable” refers to compounds that possess stability sufficient to allow manufacture and that maintain the integrity of the compound for a sufficient period of time to be useful for the purpose detailed herein.

As used herein, the following terms are intended to have the following meanings:

The term “alkoxy” refers to a group of the formula —OR^{xa}, where R^{xa} is an alkyl group. Representative alkoxy groups include methoxy, ethoxy, propoxy, tert-butoxy and the like.

The term “alkoxyalkyl” refers to an alkyl group substituted with an alkoxy group and may be represented by the general formula —R^{b}OR^{xa} where R^{b} is an alkylene group and R^{xa} is an alkyl group.

The term “alkyl” by itself or as part of another substituent refers to a saturated or unsaturated branched, straight-chain or cyclic monovalent hydrocarbon radical that is derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl, cycloprop-1-en-1-yl; cycloprop-2-en-1-yl, prop-1-yn-1-yl, prop-2-yn-1-yl, etc., butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.: and the like. Where specific levels of saturation are intended, the nomenclature “alkanyl.” “alkenyl” and/or “alkynyl” are used, as defined below. The term “lower alkyl” refers to alkyl groups with 1 to 6 carbons.

The term “alkanyl” by itself or as part of another substituent refers to a saturated branched, straight-chain or cyclic alkyl derived by the removal of one hydrogen atom from a single carbon atom of a parent alkane. Typical alkanyl groups include, but are not limited to, methyl; ethanyl; propanyls such as propan-1-yl, propan-2-yl (isopropyl), cyclopropan-1-yl, etc., butanyls such as butan-1-yl, butan-2-yl (sec-butyl), 2-methyl-propan-1-yl (isobutyl), 2-methyl-propan-2-yl (f-butyl), cyclobutan-1-yl, ere; and the like.

The term “alkenyl” by itself or as part of another substituent refers to an unsaturated branched, straight-chain or cyclic alkyl having at least one carbon-carbon double bond derived by the removal of one hydrogen atom from a single carbon atom of a parent alkene. Typical alkenyl groups include, but are not limited to, ethenyl; propenyls such as prop-1-en-1-yl, prop-1-en-2-yl, prop-2-en-1-yl, prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

The term “alkynyl” by itself or as part of another substituent refers to an un saturated branched, straight-chain or cyclic alkyl having at least one carbon-carbon triple bond derived by the removal of one hydrogen atom from a single carbon atom of a parent alkyne. Typical alkynyl groups include, but are not limited to, ethynyl; propenyls such as prop-1-yn-1-yl, prop-2-yn-1-yl, etc.; butynyls such as but-1-yn-1-yl, but-1-yn-3-yl, but-3-yn-1-yl, etc.; and the like.

The term “alkylamine” refers to a group of the formula —NHR^{xa} and “dialkylamine” refers to a group of the formula —NR^{xa} R^{xa}, where each R^{xa} is, independently of the others, an alkyl group.

The term “alkylene” refers to an alkane, alkene or alkyne group having two terminal monovalent radical centers derived by the removal of one hydrogen atom from each of the two terminal carbon atoms. Typical alkylene groups include, but are not limited to, methylene; and saturated or unsaturated ethylene: propylene; butylene; and the like. The term “lower alkylene” refers to alkylene groups with 1 to 6 carbons.

The term “heteroalkylene” refers to a divalent alkylene having one or more —CH₂— groups replaced with a thio, oxy, or —NR^{x3}— where R^{x3} is selected from hydrogen, lower alkyl and lower heteroalkyl. The heteroalkylene can be linear, branched, cyclic, bicyclic, or a combination thereof and can include up to 10 carbon atoms and up to 4 heteroatoms. The term “lower heteroalkylene” refers to alkylene groups with 1 to 4 carbon atoms and 1 to 3 heteroatoms.

The term “aryl” means an aromatic carbocyclyl containing from 6 to 14 carbon ring atoms. An aryl may be monocyclic or polycyclic (i.e., may contain more than one ring). In the case of polycyclic aromatic rings, only one ring the polycyclic system is required to be aromatic while the remaining ring(s) may be saturated, partially saturated or unsaturated. Examples of aryls include phenyl, naphthalenyl, indenyl, indanyl, and tetrahydronaphthyl.

The term “arylene” refers to an aryl group having two monovalent radical centers derived by the removal of one hydrogen atom from each of the two ring carbons. An exemplary arylene group is a phenylene.

An alkyl group may be substituted by a “carbonyl” which means that two hydrogen atoms from a single alkanylene carbon atom are removed and replaced with a double bond to an oxygen atom.

The prefix “halo” indicates that the substituent which includes the prefix is substituted with one or more independently selected halogen radicals. For example, haloalkyl means an alkyl substituent in which at least one hydrogen radical is replaced with a halogen radical. Typical halogen radicals include chloro, fluoro, bromo and iodo. Examples of haloalkyls include chloromethyl, 1-bromoethyl, fluoromethyl, difluoromethyl, trifluoromethyl, and 1,1,1-trifluoroethyl. It should be recognized that if a substituent is substituted by more than one halogen radical, those halogen radicals may be identical or different (unless otherwise stated).

The term “haloalkoxy” refers to a group of the formula —OR^{c}, where R^{c} is a haloalkyl.

The terms “heteroalkyl,” “heteroalkanyl,” “heteroalkenyl,” “heteroalkynyl,” and “heteroalkylene” refer to alkyl, alkanyl, alkenyl, alkynyl, and alkylene groups, respectively, in which one or more of the carbon atoms, e.g., 1, 2 or 3 carbon atoms, are each independently replaced with the same or different heteroatoms or heteroatomic groups. Typical heteroatoms and/or heteroatomic groups which can replace the carbon atoms include, but are not limited to, —O—, —S—, —S—O—, —NR^{c}—, —PH, —S(O)—, —S(O)₂—, —S(O)NR^{c}—, —S(O)₂NR^{c}—, and the like, including combinations thereof, where each R^{c} is independently hydrogen or C₁-C₆ alkyl. The term “lower heteroalkyl” refers to between 1 and 4 carbon atoms and between 1 and 3 heteroatoms.

The terms “cycloalkyl” and “heterocyclyl” refer to cyclic versions of “alkyl” and “heteroalkyl” groups, respectively. For heterocyclyl groups, a heteroatom can occupy the position that is attached to the remainder of the molecule. A cycloalkyl or heterocyclyl ring may be a single-ring (monocyclic) or have two or more rings (bicyclic or polycyclic).

Monocyclic cycloalkyl and heterocyclyl groups will typically contains from 3 to 7 ring atoms, more typically from 3 to 6 ring atoms, and even more typically 5 to 6 ring atoms. Examples of cycloalkyl groups include, but are not limited to, cyclopropyl; cyclobutyls such as cyclobutanyl and cyclobutenyl; cyclopentyls such as cyclopentanyl and cyclopentenyl; cyclohexyls such as cyclohexanyl and cyclohexenyl; and the like. Examples of monocyclic heterocyclyls include, but are not limited to, oxctane, furanyl, dihydrofuranyl, tetrahydrofuranyl, tetrahydropyranyl, thiophenyl (thiofuranyl), dihydrothiophenyl, tetrahydrothiophenyl, pyrrolyl, pyrrolinyl, pyrrolidinyl, imidazolyl, imidazolinyl, imidazolidinyl, pyrazolyl, pyrazolinyl, pyrazolidinyl, triazolyl, tetrazolyl, oxazolyl, oxazolidinyl, isoxazolidinyl, isoxazolyl, thiazolyl, isothiazolyl, thiazolinyl, isothiazolinyl, thiazolidinyl, isothiazolidinyl, thiodiazolyl, oxadiazolyl (including 1,2,3-oxadiazolyl, 1,2,4-oxadiazolyl, 1,2,5-oxadiazolyl (furazanyl), or 1,3,4-oxadiazolyl), oxatriazolyl (including 1,2,3,4-oxatriazolyl or 1,2,3,5-oxatriazolyl), dioxazolyl (including 1,2,3-dioxazolyl, 1,2,4-dioxazolyl, 1,3,2-dioxazolyl, or 1,3,4-dioxazolyl), 1,4-dioxanyl, dioxothiomorpholinyl, oxathiazolyl, oxathiolyl, oxathiolanyl, pyranyl, dihydropyranyl, thiopyranyl, tetrahydrothiopyranyl, pyridinyl (azinyl), piperidinyl, diazinyl (including pyridazinyl (1,2-diazinyl), pyrimidinyl (1,3-diazinyl), or pyrazinyl (1,4-diazinyl)), piperazinyl, triazinyl (including 1,3,5-triazinyl, 1,2,4-triazinyl, and 1,2,3-triazinyl)), oxazinyl (including 1,2-oxazinyl, 1,3-oxazinyl, or 1,4-oxazinyl)), oxathiazinyl (including 1,2,3-oxathiazinyl, 1,2,4-oxathiazinyl, 1,2,5-oxathiazinyl, or 1,2,6-oxathiazinyl)), oxadiazinyl (including 1,2,3-oxadiazinyl, 1,2,4-oxadiazinyl, 1,4,2-oxadiazinyl, or 1,3,5-oxadiazinyl)), morpholinyl, azepinyl, oxepinyl, thiepinyl, diazepinyl, pyridonyl (including pyrid-2(1H)-onyl and pyrid-4(1H)-onyl), furan-2(5H)-onyl, pyrimidonyl (including pyramid-2(1H)-onyl and pyramid-4(3H)-onyl), oxazol-2(3H)-onyl, 1H-imidazol-2(3H)-onyl, pyridazin-3(2H)-onyl, and pyrazin-2(1H)-onyl.

Polycyclic cycloalkyl and heterocyclyl groups contain more than one ring, and bicyclic cycloalkyl and heterocyclyl groups contain two rings. The rings may be in a bridged, fused or spiro orientation. Polycyclic cycloalkyl and heterocyclyl groups may include combinations of bridged, fused and/or spiro rings. In a spirocyclic cycloalkyl or heterocyclyl, one atom is common to two different rings. An example of a spirocycloalkyl is spiro[4.5]decane and an example of a spiroheterocyclyls is a spiropyrazoline.

In a bridged cycloalkyl or heterocyclyl, the rings share at least two common non-adjacent atoms. Examples of bridged cycloalkyls include, but are not limited to, adamantyl and norbomanyl rings. Examples of bridged heterocyclyls include, but are not limited to, 2-oxatricyclo[3.3.1.1^{3,7}]decanyl.

In a fused-ring cycloalkyl or heterocyclyl, two or more rings are fused together, such that two rings share one common bond. Examples of fused-ring cycloalkyls include decalin, naphthylene, tetralin, and anthracene. Examples of fused-ring heterocyclyls containing two or three rings include imidazopvrazinyl (including imidazo[1,2-a]pyrazinyl), imidazopyridinyl (including imidazo[1,2-a]pyridinyl), imidazopyridazinyl (including imidazo[1,2-b]pyridazinyl), thiazolopyridinyl (including thiazolo[5,4-c]pyridinyl, thiazolo[5,4-b]pyridinyl, thiazolo[4,5-b]pyridinyl, and thiazolo[4,5-c]pyridinyl), indolizinyl, pyranopyrrolyl, 4H-quinolizinyl, purinyl, naphthyridinyl, pyridopyridinyl (including pyrido[3,4-b]-pyridinyl, pyrido[3,2-b]-pyridinyl, or pyrido[4,3-b]-pyridinyl), and pteridinyl. Other examples of fused-ring hetetocyclyls include benzo-fused heterocyclyls, such as dihydrochromenyl, tetrahydroisoquinolinyl, indolyl, isoindolyl (isobenzazolyl, pseudoisoindolyl), indoleninyl (pseudoindolyl), isoindazolyl (benzpyrazolyl), benzazinyl (including quinolinyl (1-benzazinyl) or isoquinolinyl (2-benzazinyl)), phthalazinyl, quinoxalinyl, quinazolinyl, benzodiazinyl (including cinnolinyl (1,2-benzodiazinyl) or quinazolinyl (1,3-benzodiazinyl)), benzopyranyl (including chromanyl or isochromanyl), benzoxazinyl (including 1,3,2-benzoxazinyl, 1,4,2-benzoxazinyl, 2,3,1-benzoxazinyl, or 3,1,4-benzoxazinyl), benzo[d]thiazolyl and benzisoxazinyl (including 1,2-benzisoxazinyl or 1,4-benzisoxazinyl).

The term “heteroaryl” refers to an aromatic heterocyclyl containing from 5 to 14 ring atoms. A heteroaryl may be a single ring or 2 or 3 fused rings. Examples of heteroaryls include 6-membered rings such as pyridyl, pyrazyl, pyrimidinyl, pyridazinyl, and 1,3,5-, 1,2,4- or 1,2,3-triazinyl; 5-membered ring substituents such as triazolyl, pyrrolyl, imidazyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as imidazopyrazinyl (including imidazo[1,2-a]pyrazinyl)imidazopyridinyl (including imidazo[1,2-a]pyridinyl), imidazopyridazinyl (including imidazo[1,2-b]pyridazinyl), thiazolopyridinyl (including thiazolo[5,4-c]pyridinyl, thiazolo[5,4-b]pyridinyl, thiazolo[4,5-b]pyridinyl, and thiazolo[4,5-c]pyridinyl), benzo[d]thiazolyl, benzothiofuranyl, benzisoxazolyl, benzoxazolyl, purinyl, and anthranilyl; and 6/6-membered fused rings such as benzopyranyl, quinolinyl, isoquinolinyl, cinnolinyl, quinazolinyl, and benzoxazinyl. Heteroaryls may also be heterocycles having aromatic (4N+2 pi electron) resonance contributors such as pyridonyl (including pyrid-2(1H)-onyl and pyrid-4(1H)-onyl), pyrimidonyl (including pyramid-2(1H)-onyl and pyramid-4(3H)-onyl), pyridazin-3(2H)-onyl and pyrazin-2(1H)-onyl.

The term “sulfonate” as used herein means a salt or ester of a sulfonic acid.

The term “methyl sulfonate” as used herein means a methyl ester of a sulfonic acid group.

The term “carboxylate” as used herein means a salt or ester of a carboxylic acid.

The term “polyol”, as used herein, means a group containing more than two hydroxyl groups independently or as a portion of a monomer unit. Polyols include, but are not limited to, reduced C₂-C₆ carbohydrates, ethylene glycol, and glycerin.

The term “sugar” when used in context of “G¹” includes O-glycoside, —Y-glycoside, S-glycoside and C-glycoside (C-glycosyl) carbohydrate derivatives of the monosaccharide and disaccharide classes and may originate from naturally-occurring sources or may be synthetic in origin. For example “sugar” when used in context of “G¹” includes derivatives such as but not limited to those derived from glucuronic acid, galacturonic acid, galactose, and glucose among others. Suitable sugar substitutions include but are not limited to hydroxyl, amine, carboxylic acid, sulfonic acid, phosphonic acid, esters, and ethers.

The term “NHS ester” means the N-hydroxysuccinimide ester derivative of a carboxylic acid.

The term “amine” includes primary, secondary and tertiary aliphatic amines, including cyclic versions.

The term salt when used in context of “or salt thereof” include salts commonly used to form alkali metal salts and to form addition salts of free acids or free bases. In general, these salts typically may be prepared by conventional means by reacting, for example, the appropriate acid or base with a compound of the invention

Where a salt is intended to be administered to a patient (as opposed to, for example, being in use in an in vitro context), the salt preferably is pharmaceutically acceptable and/or physiologically compatible. The term “pharmaceutically acceptable” is used adjectivally in this patent application to mean that the modified noun is appropriate for use as a pharmaceutical product or as a part of a pharmaceutical product. The term “pharmaceutically acceptable salt” includes salts commonly used to form alkali metal salts and to form addition salts of free acids or free bases. In general, these salts typically may be prepared by conventional means by reacting, for example, the appropriate acid or base with a compound of the invention.

The term “drug-to-antibody ratio” or “DAR” refers to the number of drugs, e.g., a Bcl-xL inhibitor, attached to the antibody of the ADC. The DAR of an ADC can range from 1 to 8, although higher loads, e.g., 20, are also possible depending on the number of linkage site on an antibody. The term DAR may be used in reference to the number of drugs loaded onto an individual antibody, or, alternatively, may be used in reference to the average or mean DAR of a group of ADCs.

The term “undesired ADC species”, as used herein, refers to any drug loaded species which is to be separated from an ADC species having a different drug load. In one embodiment, the term undesired ADC species may refer to drug loaded species of 6 or more, i.e., ADCs with a DAR of 6 or more, including DAR6, DAR7, DAR8, and DAR greater than 8 (i.e., drug loaded species of 6, 7, 8, or greater than 8). In a separate embodiment, the term undesired ADC species may refer to drug loaded species of 8 or more, i.e., ADCs with a DAR of 8 or more, including DAR8, and DAR greater than 8 (i.e., drug loaded species of 8, or greater than 8).

The term “ADC mixture”, as used herein, refers to a composition containing a heterogeneous DAR distribution of ADCs. In one embodiment, an ADC mixture contains ADCs having a distribution of DARs of 1 to 8, e.g., 2, 4, 6, and 8 (i.e., drug loaded species of 2, 4, 6, and 8). Notably, degradation products may result such that DARs of 1, 3, 5, and 7 may also be included in the mixture. Further. ADCs within the mixture may also have DARs greater than 8. The ADC mixture results from interchain disulfide reduction followed by conjugation. In one embodiment, the ADC mixture comprises both ADCs with a DAR of 4 or less (i.e., a drug loaded species of 4 or less) and ADCs with a DAR of 6 or more (i.e., a drug loaded species of 6 or more).

The term “cancer” is meant to refer to or describe the physiological condition in mammals that is typically characterized by unregulated cell growth. Examples of cancer include, but are not limited to, carcinoma, lymphoma, blastema, sarcoma, and leukemia or lymphoid malignancies. More particular examples of such cancers include glioblastoma, small cell lung cancer, non-small cell lung cancer, lung cancer, colon cancer, colorectal cancer, head and neck cancer, breast cancer (e.g., triple negative breast cancer), pancreatic cancer, squamous cell tumors, squamous cell carcinoma (e.g., squamous cell lung cancer or squamous cell head and neck cancer), anal cancer, skin cancer, vulvar cancer, multiple myeloma, acute myeloid leukemia. In one embodiment, the antibodies or ADCs of the invention are administered to a patient having a tumor(s) containing amplifications of the CD98 gene. In one embodiment the antibodies or ADCs of the invention are administered to a patient having a solid tumor which is likely to overexpress CD98 In one embodiment, the antibodies or ADCs of the invention are administered to a patient having squamous cell Non-Small Cell Lung Cancer (NSCLC). In one embodiment, the antibodies or ADCs of the invention are administered to a patient having small cell lung cancer. In another embodiment, the antibodies or ADCs of the invention are administered to a patient having breast cancer. In another embodiment, the antibodies or ADCs of the invention are administered to a patient having ovarian cancer. In another embodiment, the antibodies or ADCs of the invention are administered to a patient having multiple myeloma. In another embodiment, the antibodies or ADCs of the invention are administered to a patient having acute myeloid leukemia. In one embodiment the antibodies or ADCs of the invention are administered to a patient having solid tumors, including advanced solid tumors. In certain embodiments, the antibodies or ADCs of the invention are administered to a patient having cancer that is characterized as having EGFR overexpression. In other embodiments, the antibodies or ADCs of the invention are administered to a patient having cancer that is characterized by an activating EGFR mutation, e.g., a mutation(s) that activates the EGFR signaling pathway and/or mutation(s) that lead to overexpression of the EGFR protein. In specific exemplary embodiments, the activating EGFR mutation may be a mutation in the EGFR gene. In particular embodiments, the activating EGFR mutation is an exon 19 deletion mutation, a single-point substitution mutation L858R in exon 21, a T790M point mutation, and/or combinations thereof.

The term “CD98 expressing tumor.” as used herein, refers to a tumor which expresses CD98 protein. In one embodiment, CD98 expression in a tumor is determined using immunohistochemical staining of tumor cell membranes, where any immunohistochemical staining above background level in a tumor sample indicates that the tumor is a CD98 expressing tumor. Methods for detecting expression of CD98 in a tumor are known in the art, e.g., the CD98 pharmDx™ Kit (Dako). In contrast a “CD98 negative tumor” is defined as a tumor having an absence of CD98 membrane staining above background in a tumor sample as determined by immunohistochemical techniques.

The terms “overexpress,” “overexpression,” or “overexpressed” interchangeably refer to a gene that is transcribed or translated at a delectably greater level, usually in a cancer cell, in comparison to a normal cell. Overexpression therefore refers to both overexpression of protein and RNA (due to increased transcription, post transcriptional processing, translation, post translational processing, altered stability, and altered protein degradation), as well as local overexpression due to altered protein traffic patterns (increased nuclear localization), and augmented functional activity, e.g., as in an increased enzyme hydrolysis of substrate. Thus, overexpression refers to either protein or RNA levels. Overexpression can also be by 50%, 60%, 70%, 80%, 90% or more in comparison to a normal cell or comparison cell. In certain embodiments, the anti-CD98 antibodies or ADCs of the invention are used to treat solid tumors likely to overexpress CD98.

The term “gene amplification”, as used herein, refers to a cellular process characterized by the production of multiple copies of any particular piece of DNA. For example, a tumor cell may amplify, or copy, chromosomal segments as a result of cell signals and sometimes environmental events. The process of gene amplification leads to the production of additional copies of the gene. In one embodiment, the gene is CD98, i.e., “CD98 amplification.” In one embodiment, the compositions and methods disclosed herein are used to treat a subject having CD98 amplified cancer.

The term “administering” as used herein is meant to refer to the delivery of a substance (e.g., an anti-CD98 antibody or ADC) to achieve a therapeutic objective (e.g., the treatment of a CD98-associated disorder). Modes of administration may be parenteral, enteral and topical. Parenteral administration is usually by injection, and includes, without limitation, intravenous, intramuscular, intraarterial, intrathecal intracapsular, intraorbital, intracardiac, intradermal, intraperitoneal, transtracheal, subcutaneous, subcuticular, intraarticular, subcapsular, subarachnoid, intraspinal and intrasternal injection and infusion.

The term “combination therapy”, as used herein, refers to the administration of two or more therapeutic substances, e.g., an anti-CD98 antibody or ADC and an additional therapeutic agent. The additional therapeutic agent may be administered concomitant with, prior to, or following the administration of the anti-CD98 antibody or ADC.

As used herein, the term “effective amount” or “therapeutically effective amount” refers to the amount of a drug, e.g., an antibody or ADC, which is sufficient to reduce or ameliorate the severity and/or duration of a disorder, e.g., cancer, or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent). The effective amount of an antibody or ADC may, for example, inhibit tumor growth (e.g., inhibit an increase in tumor volume), decrease tumor growth (e.g., decrease tumor volume), reduce the number of cancer cells, and/or relieve to some extent one or more of the symptoms associated with the cancer. The effective amount may, for example, improve disease free survival (DFS), improve overall survival (OS), or decrease likelihood of recurrence.

The term a “xenograft assay”, as used herein, refers to a human tumor xenograft assay, wherein human tumor cells are transplanted, either under the skin or into the organ type in which the tumor originated, into immunocompromised mice that do not reject human cells.

Various aspects of the invention are described in further detail in the following subsections.

### II. Anti-CD98 Antibodies

The invention is based, at least in part, on the identification of humanized anti-CD98 antibodies. In one embodiment, the present invention provides murine anti-CD98 antibodies, or antigen binding portions thereof. In another embodiment, the present invention provides chimeric anti-CD98 antibodies, or antigen binding portions thereof. In yet another embodiment, the present invention provides humanized anti-CD98 antibodies, or antigen binding portions thereof. In another aspect of the invention features antibody drug conjugates (ADCs) comprising an anti-CD98 antibody described herein and at least one drug(s), such as, but not limited to, a Bcl-xL inhibitor. The antibodies or ADCs of the invention have characteristics including, but not limited to, binding to wild-type CD98 in vitro, binding to wild-type CD98 on tumor cells expressing CD98, and decreasing or inhibiting tumor cellular proliferation or tumor growth.

One aspect of the invention features an anti-human CD98 (anti-hCD98) Antibody Drug Conjugate (ADC) comprising an anti-hCD98 antibody conjugated to a drug via a linker, wherein the drug is a Bcl-xL inhibitor. Exemplary anti-CD98 antibodies (and sequences thereof) that can be used in the ADCs described herein.

The anti-CD98 antibodies described herein provide the ADCs of the invention with the ability to bind to CD98 such that the cytotoxic Bcl-xL drug attached to the antibody may be delivered to the CD98-expressing cell, particularly a CD98 expressing cancer cell.

While the term “antibody” is used throughout, it should be noted that antibody fragments (i.e., antigen-binding portions of an anti-CD98 antibody) are also included in the invention and may be included in the embodiments (methods and compositions) described throughout. For example, an anti-CD98 antibody fragment may be conjugated to the Bcl-xL inhibitors described herein. In certain embodiments, an anti-CD98 antibody binding portion is a Fab, a Fab′, a F(ab′)₂, a Fv, a disulfide linked Fv, an scFv, a single domain antibody, or a diabody.

### II.A. Anti-CD98 Chimeric Antibodies

A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al. (1989) J. Immunol. Methods 125:191-202; U.S. Pat. Nos. 5,807,715; 4,816,567; and 4,816,397, which are incorporated herein by reference in their entireties. In addition, techniques developed for the production of “chimeric antibodies” (Morrison et al., 1984. Proc. Natl. Acad Set. 81:851-855; Neuberger et al, 1984, Nature 312:604-608; Takeda et al. 1985, Nature 314:452-454, each of which are incorporated herein by reference in their entireties) by splicing genes from a mouse antibody molecule of appropriate antigen specificity together with genes from a human antibody molecule of appropriate biological activity can be used.

As described in Example 3, fifteen anti-hCD98 murine antibodies were identified, i.e., Ab1-Ab15 (mouse antibodies Ab1, Ab2, Ab3, Ab4, and Ab5 and rat antibodies Ab6, Ab7, Ab8, Ab9, Ab10, Ab11, Ab12, Ab13, Ab14, and Ab15). The variable regions from these antibodies were sequenced and combined with human IgG1 sequences to form chimeric antibodies as described in Example 5.

Recombinant anti-CD98 chimeric antibodies corresponding to murine antibodies Ab1, Ab2, Ab3, Ab4, and Ab5, Ab6, Ab7, Ab8, Ab9, Ab10, Ab11, Ab12, Ab13, Ab14, and Ab15 were produced and include human IgG1 heavy chain and kappa light chain constant regions (described below in Example 5). These chimeric antibodies are identified in Table 5 as chAb1, chAb2, chAb3, chAb4, and chAb5, chAb6, chAb7, chAb8, chAb9, chAb10, chAb11, chAb12, chAb13, chAb14, and chAb15. Tables 6 and 7 provide the amino acid sequences of CDR, VH, and VL regions of chimeric antibodies chAb1, chAb2, chAb3, chAb4, and chAb5, chAb6, chAb7, chAb8, chAb9, chAb10, chAb11, chAb12, chAb13, chAb14, and chAb15.

Thus, in one aspect, the present invention is directed to an anti-CD98 antibody, or antigen-binding portion thereof, having a heavy chain variable region including an amino acid sequence set forth in SEQ ID NOs: 1, 9, 15, 20, 23, 28, 35, 39, 47, 52, 56, 60, 63, 70 or 78; and/or a light chain variable region including an amino acid sequence set forth in SEQ ID NOs: 5, 12, 18, 22, 26, 32, 38, 43, 49, 55, 58, 62, 67, 74, or 82.

In another aspect, the present invention is directed to an anti-CD98 antibody, or antigen-binding portion thereof, having a heavy chain variable region including an amino acid sequence as set forth in SEQ ID NO: 1, and a light chain variable region including an amino acid sequence set forth in SEQ ID NO: 5.

In another aspect, the present invention is directed to an anti-CD98 antibody, or antigen-binding portion thereof, having a heavy chain variable domain region including (a) a CDR1 having an amino acid sequence as set forth in SEQ ID NO: 2; (b) a CDR2 having an amino acid sequence as set forth in SEQ ID NO: 3; and (c) a CDR3 having an amino acid sequence as set forth in SEQ ID NO: 4; and a light chain variable region including (a) a CDR1 having an amino acid sequence as set forth in SEQ ID NO: 6; (b) a CDR2 having an amino acid sequence as set forth in SEQ ID NO: 7; and (c) a CDR3 having an amino acid sequence as set forth in SEQ ID NO: 8.

In another aspect, the present invention is directed to an anti-CD98 antibody, or antigen-binding portion thereof, having a heavy chain variable region including an amino acid sequence as set forth in SEQ ID NO: 9, and a light chain variable region including an amino acid sequence set forth in SEQ ID NO: 12.

In another aspect the present invention is directed to an anti-CD98 antibody, or antigen-binding portion thereof, having a heavy chain variable domain region including (a) a CDR1 having an amino acid sequence as set forth in SEQ ID NO: 10; (b) a CDR2 having an amino acid sequence as set forth in SEQ ID NO: 11; and (c) a CDR3 having an amino acid sequence as set forth in SEQ ID NO: 4; and a light chain variable region including (a) a CDR1 having an amino acid sequence as set forth in SEQ ID NO: 13 (b) a CDR2 having an amino acid sequence as set forth in SEQ ID NO: 7; and (c) a CDR3 having an amino acid sequence as set forth in SEQ ID NO: 14.

In another aspect, the present invention is directed to an anti-CD98 antibody, or antigen-binding portion thereof, having a heavy chain variable region including an amino acid sequence as set forth in SEQ ID NO: 15, and a light chain variable region including an amino acid sequence set forth in SEQ ID NO: 18.

In another aspect, the present invention is directed to an anti-CD98 antibody, or antigen-binding portion thereof, having a heavy chain variable domain region including (a) a CDR1 having an amino acid sequence as set forth in SEQ ID NO: 16; (b) a CDR2 having an amino acid sequence as set forth in SEQ ID NO: 11; and (c) a CDR3 having an amino acid sequence as set forth in SEQ ID NO: 17; and a light chain variable region including (a) a CDR1 having an amino acid sequence as set forth in SEQ ID NO: 13; (b) a CDR2 having an amino acid sequence as set forth in SEQ ID NO: 7; and (c) a CDR3 having an amino acid sequence as set forth in SEQ ID NO: 19.

In another aspect, the present invention is directed to an anti-CD98 antibody, or antigen-binding portion thereof, having a heavy chain variable region including an amino acid sequence as set forth in SEQ ID NO: 20, and a light chain variable region including an amino acid sequence set forth in SEQ ID NO: 22.

In another aspect, the present invention is directed to an anti-CD98 antibody, or antigen-binding portion thereof, having a heavy chain variable domain region including (a) a CDR1 having an amino acid sequence as set forth in SEQ ID NO: 2; (b) a CDR2 having an amino acid sequence as set forth in SEQ ID NO: 21; and (c) a CDR3 having an amino acid sequence as set forth in SEQ ID NO: 4; and a light chain variable region including (a) a CDR1 having an amino acid sequence as set forth in SEQ ID NO: 13; (b) a CDR2 having an amino acid sequence as set forth in SEQ ID NO: 7; and (c) a CDR3 having an amino acid sequence as set forth in SEQ ID NO: 8.

In another aspect, the present invention is directed to an anti-CD98 antibody, or antigen-binding portion thereof, having a heavy chain variable region including an amino acid sequence as set forth in SEQ ID NO: 23, and a light chain variable region including an amino acid sequence set forth in SEQ ID NO: 26.

In another aspect, the present invention is directed to an anti-CD98 antibody, or antigen-binding portion thereof, having a heavy chain variable domain region including (a) a CDR1 having an amino acid sequence as set forth in SEQ ID NO: 24; (b) a CDR2 having an amino acid sequence as set forth in SEQ ID NO: 11; and (c) a CDR3 having an amino acid sequence as set forth in SEQ ID NO: 25; and a light chain variable region including (a) a CDR1 having an amino acid sequence as set forth in SEQ ID NO: 13; (b) a CDR2 having an amino acid sequence as set forth in SEQ ID NO: 7; and (c) a CDR3 having an amino acid sequence as set forth in SEQ ID NO: 27.

In another aspect, the present invention is directed to an anti-CD98 antibody, or antigen-binding portion thereof, having a heavy chain variable region including an amino acid sequence as set forth in SEQ ID NO: 28, and a light chain variable region including an amino acid sequence set forth in SEQ ID NO: 32.

In another aspect the present invention is directed to an anti-CD98 antibody, or antigen-binding portion thereof, having a heavy chain variable domain region including (a) a CDR1 having an amino acid sequence as set forth in SEQ ID NO: 29; (b) a CDR2 having an amino acid sequence as set forth in SEQ ID NO: 30; and (c) a CDR3 having an amino acid sequence as set forth in SEQ ID NO: 31; and a light chain variable region including (a) a CDR1 having an amino acid sequence as set forth in SEQ ID NO: 33; (b) a CDR2 having an amino acid sequence as set forth in SEQ ID NO: 7; and (c) a CDR3 having an amino acid sequence as set forth in SEQ ID NO: 34.

In another aspect the present invention is directed to an anti-CD98 antibody, or antigen-binding portion thereof, having a heavy chain variable region including an amino acid sequence as set forth in SEQ ID NO: 35, and a light chain variable region including an amino acid sequence set forth in SEQ ID NO: 38.

In another aspect, the present invention is directed to an anti-CD98 antibody, or antigen-binding portion thereof, having a heavy chain variable domain region including (a) a CDR1 having an amino acid sequence as set forth in SEQ ID NO: 29; (b) a CDR2 having an amino acid sequence as set forth in SEQ ID NO: 36; and (c) a CDR3 having an amino acid sequence as set forth in SEQ ID NO: 37; and a light chain variable region including (a) a CDR1 having an amino acid sequence as set forth in SEQ ID NO: 33; (b) a CDR2 having an amino acid sequence as set forth in SEQ ID NO: 7; and (c) a CDR3 having an amino acid sequence as set forth in SEQ ID NO: 34.

In another aspect, the present invention is directed to an anti-CD98 antibody, or antigen-binding portion thereof, having a heavy chain variable region including an amino acid sequence as set forth in SEQ ID NO: 39, and a light chain variable region including an amino acid sequence set forth in SEQ ID NO: 43.

In another aspect, the present invention is directed to an anti-CD98 antibody, or antigen-binding portion thereof, having a heavy chain variable domain region including (a) a CDR1 having an amino acid sequence as set forth in SEQ ID NO: 40; (b) a CDR2 having an amino acid sequence as set forth in SEQ ID NO: 41; and (c) a CDR3 having an amino acid sequence as set forth in SEQ ID NO: 42; and a light chain variable region including (a) a CDR1 having an amino acid sequence as set forth in SEQ ID NO: 44; (b) a CDR2 having an amino acid sequence as set forth in SEQ ID NO: 45; and (c) a CDR3 having an amino acid sequence as set forth in SEQ ID NO: 46.

In another aspect, the present invention is directed to an anti-CD98 antibody, or antigen-binding portion thereof, having a heavy chain variable region including an amino acid sequence as set forth in SEQ ID NO: 47, and a light chain variable region including an amino acid sequence set forth in SEQ ID NO: 49.

In another aspect, the present invention is directed to an anti-CD98 antibody, or antigen-binding portion thereof, having a heavy chain variable domain region including (a) a CDR1 having an amino acid sequence as set forth in SEQ ID NO: 48; (b) a CDR2 having an amino acid sequence as set forth in SEQ ID NO: 30; and (c) a CDR3 having an amino acid sequence as set forth in SEQ ID NO: 37; and a light chain variable region including (a) a CDR1 having an amino acid sequence as set forth in SEQ ID NO: 50; (b) a CDR2 having an amino acid sequence as set forth in SEQ ID NO: 7; and (c) a CDR3 having an amino acid sequence as set forth in SEQ ID NO: 51.

In another aspect the present invention is directed to an anti-CD98 antibody, or antigen-binding portion thereof, having a heavy chain variable region including an amino acid sequence as set forth in SEQ ID NO: 52, and a light chain variable region including an amino acid sequence set forth in SEQ ID NO: 55.

In another aspect, the present invention is directed to an anti-CD98 antibody, or antigen-binding portion thereof, having a heavy chain variable domain region including (a) a CDR1 having an amino acid sequence as set forth in SEQ ID NO: 40; (b) a CDR2 having an amino acid sequence as set forth in SEQ ID NO: 53; and (c) a CDR3 having an amino acid sequence as set forth in SEQ ID NO: 54; and a light chain variable region including (a) a CDR1 having an amino acid sequence as set forth in SEQ ID NO: 44; (b) a CDR2 having an amino acid sequence as set forth in SEQ ID NO: 45; and (c) a CDR3 having an amino acid sequence as set forth in SEQ ID NO: 46.

In another aspect, the present invention is directed to an anti-CD98 antibody, or antigen-binding portion thereof, having a heavy chain variable region including an amino acid sequence as set forth in SEQ ID NO: 56, and a light chain variable region including an amino acid sequence set forth in SEQ ID NO: 58.

In another aspect, the present invention is directed to an anti-CD98 antibody, or antigen-binding portion thereof, having a heavy chain variable domain region including (a) a CDR1 having an amino acid sequence as set forth in SEQ ID NO: 40; (b) a CDR2 having an amino acid sequence as set forth in SEQ ID NO: 57; and (c) a CDR3 having an amino acid sequence as set forth in SEQ ID NO: 42; and a light chain variable region including (a) a CDR1 having an amino acid sequence as set forth in SEQ ID NO: 59; (b) a CDR2 having an amino acid sequence as set forth in SEQ ID NO: 45; and (c) a CDR3 having an amino acid sequence as set forth in SEQ ID NO: 46.

In another aspect, the present invention is directed to an anti-CD98 antibody, or antigen-binding portion thereof, having a heavy chain variable region including an amino acid sequence as set forth in SEQ ID NO: 60, and a light chain variable region including an amino acid sequence set forth in SEQ ID NO: 62.

In another aspect the present invention is directed to an anti-CD98 antibody, or antigen-binding portion thereof, having a heavy chain variable domain region including (a) a CDR1 having an amino acid sequence as set forth in SEQ ID NO: 40; (b) a CDR2 having an amino acid sequence as set forth in SEQ ID NO: 41; and (c) a CDR3 having an amino acid sequence as set forth in SEQ ID NO: 61; and a light chain variable region including (a) a CDR1 having an amino acid sequence as set forth in SEQ ID NO: 44; (b) a CDR2 having an amino acid sequence as set forth in SEQ ID NO: 45; and (c) a CDR3 having an amino acid sequence as set forth in SEQ ID NO: 46.

In another aspect, the present invention is directed to an anti-CD98 antibody, or antigen-binding portion thereof, having a heavy chain variable region including an amino acid sequence as set forth in SEQ ID NO: 63, and a light chain variable region including an amino acid sequence set forth in SEQ ID NO: 67.

In another aspect, the present invention is directed to an anti-CD98 antibody, or antigen-binding portion thereof, having a heavy chain variable domain region including (a) a CDR1 having an amino acid sequence as set forth in SEQ ID NO: 64; (b) a CDR2 having an amino acid sequence as set forth in SEQ ID NO: 65; and (c) a CDR3 having an amino acid sequence as set forth in SEQ ID NO: 66; and a light chain variable region including (a) a CDR1 having an amino acid sequence as set forth in SEQ ID NO: 68; (b) a CDR2 having an amino acid sequence as set forth in SEQ ID NO: 7; and (c) a CDR3 having an amino acid sequence as set forth in SEQ ID NO: 69.

In another aspect, the present invention is directed to an anti-CD98 antibody, or antigen-binding portion thereof, having a heavy chain variable region including an amino acid sequence as set forth in SEQ ID NO: 70, and a light chain variable region including an amino acid sequence set forth in SEQ ID NO: 74.

In another aspect, the present invention is directed to an anti-CD98 antibody, or antigen-binding portion thereof, having a heavy chain variable domain region including (a) a CDR1 having an amino acid sequence as set forth in SEQ ID NO: 71; (b) a CDR2 having an amino acid sequence as set forth in SEQ ID NO: 72; and (c) a CDR3 having an amino acid sequence as set forth in SEQ ID NO: 73; and a light chain variable region including (a) a CDR1 having an amino acid sequence as set forth in SEQ ID NO: 75 (b) a CDR2 having an amino acid sequence as set forth in SEQ ID NO: 76; and (c) a CDR3 having an amino acid sequence as set forth in SEQ ID NO: 77.

In another aspect, the present invention is directed to an anti-CD98 antibody, or antigen-binding portion thereof, having a heavy chain variable region including an amino acid sequence as set forth in SEQ ID NO: 78, and a light chain variable region including an amino acid sequence set forth in SEQ ID NO: 82.

In another aspect, the present invention is directed to an anti-CD98 antibody, or antigen-binding portion thereof, having a heavy chain variable domain region including (a) a CDR1 having an amino acid sequence as set forth in SEQ ID NO: 79; (b) a CDR2 having an amino acid sequence as set forth in SEQ ID NO: 80; and (c) a CDR3 having an amino acid sequence as set forth in SEQ ID NO: 81; and a light chain variable region including (a) a CDR1 having an amino acid sequence as set forth in SEQ ID NO: 83; (b) a CDR2 having an amino acid sequence as set forth in SEQ ID NO: 45; and (c) a CDR3 having an amino acid sequence as set forth in SEQ ID NO: 84.

### II.B. Humanized Anti-CD98 Antibodies

Following the production of chimeric antibodies chAb1, chAb2, chAb3, chAb4, and chAb5, chAb6, chAb7, chAb8, chAb9, chAb10, chAb11, chAb12, chAb13, chAb14, and chAb15, antibodies chAb3 and chAb15 were selected for humanization (described below in Example 12), resulting in the production of humanized antibodies huAb3 and huAb15.

The heavy chain variable sequence of huAb3 is provided in SEQ ID NO: 85 with CDR1. CDR2, and CDR3 sequences described in SEQ ID NOs: 16, 11, and 17 respectively. The light chain variable sequence of huAb3 is provided in SEQ ID NO: 88 with CDR1, CDR2, and CDR3 sequences described in SEQ ID NOs: 13, 7, and 19, respectively.

The heavy chain variable sequence of huAb15 is provided in SEQ ID NO: 122 with CDR1, CDR2, and CDR3 sequences described in SEQ ID NOs: 79.80, and 81, respectively. The light chain variable sequence of huAb15 is provided in SEQ ID NO: 123 with CDR1, CDR2, and CDR3 sequences described in SEQ ID NOs: 83, 45, and 84, respectively.

huAb3 and huAb15 were modified to remove specific amino acids contained in the variable regions, as described in Example 12 in order to remove post-translational modifications that had the potential to reduce affinity, potency, stability and/or homogeneity of the antibody. Variants of huAb3 and huAb1S were generated containing point mutations at each of the identified amino acids, including all possible amino acids except M, C, N, D, G, S, or P. Specifically, two different humanized antibodies were created based on chAb3, and are referred to herein as huAb3v1, huAb3v2, and seven different humanized antibodies were created based on chAb15, and are referred to herein as huAb15v1, huAb15v2, huAb15v3, huAb15v4, huAb15v5, huAb15v6, and huAb15v7 (sec Examples and 11). Humanized antibodies huAb3v1, huAb3v2, huAb15v1, huAb15v2, huAb15v3, huAb15v4, huAb15v5, huAb15v6, and huAb15v7, which maintained binding to human CD98, are listed in Table 14. The CDR, VH, and VL amino acid sequences of huAb3v1, huAb3v2, huAb15v1, huAb15v2, huAb15v3, huAb15v4, huAb15v5, huAb15v6, and huAb15v7 mAbs are listed in Table 15.

Thus, in one aspect, the present invention is directed to an anti-CD98 antibody, or antigen-binding portion thereof, having a heavy chain variable region including an amino acid sequence set forth in SEQ ID NOs: 83, 85, 89, 91, 96, 99, 103, or 122; and/or a light chain variable region including an amino acid sequence set forth in SEQ ID NOs: 88, 94, 98, 101, or 123.

In another aspect, the present invention is directed to an anti-CD98 antibody, or antigen-binding portion thereof, having a heavy chain variable region including an amino acid sequence as set forth in SEQ ID NO: 85, and a light chain variable region including an amino acid sequence set forth in SEQ ID NO: 88.

In another aspect, the present invention is directed to an anti-CD98 antibody, or antigen-binding portion thereof, having a heavy chain variable domain region including (a) a CDR1 having an amino acid sequence as set forth in SEQ ID NO: 16; (b) a CDR2 having an amino acid sequence as set forth in SEQ ID NO: 11; and (c) a CDR3 having an amino acid sequence as set forth in SEQ ID NO: 17; and a light chain variable region including (a) a CDR1 having an amino acid sequence as set forth in SEQ ID NO: 13; (b) a CDR2 having an amino acid sequence as set forth in SEQ ID NO: 7; and (c) a CDR3 having an amino acid sequence as set forth in SEQ ID NO: 19.

In another aspect, the present invention is directed to an anti-CD98 antibody, or antigen-binding portion thereof, having a heavy chain variable region including an amino acid sequence as set forth in SEQ ID NO: 122, and a light chain variable region including an amino acid sequence set forth in SEQ ID NO: 123.

In another aspect, the present invention is directed to an anti-CD98 antibody, or antigen-binding portion thereof, having a heavy chain variable domain region including (a) a CDR1 having an amino acid sequence as set forth in SEQ ID NO: 79; (b) a CDR2 having an amino acid sequence as set forth in SEQ ID NO: 80; and (c) a CDR3 having an amino acid sequence as set forth in SEQ ID NO: 81; and a light chain variable region including (a) a CDR1 having an amino acid sequence as set forth in SEQ ID NO: 83 (b) a CDR2 having an amino acid sequence as set forth in SEQ ID NO: 45; and (c) a CDR3 having an amino acid sequence as set forth in SEQ ID NO: 84.

In another aspect, the present invention is directed to an anti-CD98 antibody, or antigen-binding portion thereof, having a heavy chain variable region including an amino acid sequence as set forth in SEQ ID NO: 83, and a light chain variable region including an amino acid sequence set forth in SEQ ID NO: 88.

In another aspect the present invention is directed to an anti-CD98 antibody, or antigen-binding portion thereof, having a heavy chain variable domain region including (a) a CDR1 having an amino acid sequence as set forth in SEQ ID NO: 16; (b) a CDR2 having an amino acid sequence as set forth in SEQ ID NO: 87; and (c) a CDR3 having an amino acid sequence as set forth in SEQ ID NO: 17; and a light chain variable region including (a) a CDR1 having an amino acid sequence as set forth in SEQ ID NO: 13 (b) a CDR2 having an amino acid sequence as set forth in SEQ ID NO: 7; and (c) a CDR3 having an amino acid sequence as set forth in SEQ ID NO: 19.

In another aspect the present invention is directed to an anti-CD98 antibody, or antigen-binding portion thereof, having a heavy chain variable region including an amino acid sequence as set forth in SEQ ID NO: 89, and a light chain variable region including an amino acid sequence set forth in SEQ ID NO: 88.

In another aspect, the present invention is directed to an anti-CD98 antibody, or antigen-binding portion thereof, having a heavy chain variable domain region including (a) a CDR1 having an amino acid sequence as set forth in SEQ ID NO: 16; (b) a CDR2 having an amino acid sequence as set forth in SEQ ID NO: 90; and (c) a CDR3 having an amino acid sequence as set forth in SEQ ID NO: 17; and a light chain variable region including (a) a CDR1 having an amino acid sequence as set forth in SEQ ID NO: 13 (b) a CDR2 having an amino acid sequence as set forth in SEQ ID NO: 7; and (c) a CDR3 having an amino acid sequence as set forth in SEQ ID NO: 19.

In another aspect, the present invention is directed to an anti-CD98 antibody, or antigen-binding portion thereof, having a heavy chain variable region including an amino acid sequence as set forth in SEQ ID NO: 91, and a light chain variable region including an amino acid sequence set forth in SEQ ID NO: 94.

In another aspect, the present invention is directed to an anti-CD98 antibody, or antigen-binding portion thereof, having a heavy chain variable domain region including (a) a CDR1 having an amino acid sequence as set forth in SEQ ID NO: 79; (b) a CDR2 having an amino acid sequence as set forth in SEQ ID NO: 92; and (c) a CDR3 having an amino acid sequence as set forth in SEQ ID NO: 93; and a light chain variable region including (a) a CDR1 having an amino acid sequence as set forth in SEQ ID NO: 83 (b) a CDR2 having an amino acid sequence as set forth in SEQ ID NO: 45; and (c) a CDR3 having an amino acid sequence as set forth in SEQ ID NO: 95.

In another aspect, the present invention is directed to an anti-CD98 antibody, or antigen-binding portion thereof, having a heavy chain variable region including an amino acid sequence as set forth in SEQ ID NO: 96, and a light chain variable region including an amino acid sequence set forth in SEQ ID NO: 94.

In another aspect, the present invention is directed to an anti-CD98 antibody, or antigen-binding portion thereof, having a heavy chain variable domain region including (a) a CDR1 having an amino acid sequence as set forth in SEQ ID NO: 79; (b) a CDR2 having an amino acid sequence as set forth in SEQ ID NO: 92; and (c) a CDR3 having an amino acid sequence as set forth in SEQ ID NO: 97; and a light chain variable region including (a) a CDR1 having an amino acid sequence as set forth in SEQ ID NO: 83 (b) a CDR2 having an amino acid sequence as set forth in SEQ ID NO: 45; and (c) a CDR3 having an amino acid sequence as set forth in SEQ ID NO: 95.

In another aspect, the present invention is directed to an anti-CD98 antibody, or antigen-binding portion thereof, having a heavy chain variable region including an amino acid sequence as set forth in SEQ ID NO: 96, and a light chain variable region including an amino acid sequence set forth in SEQ ID NO: 98.

In another aspect the present invention is directed to an anti-CD98 antibody, or antigen-binding portion thereof, having a heavy chain variable domain region including (a) a CDR1 having an amino acid sequence as set forth in SEQ ID NO: 79; (b) a CDR2 having an amino acid sequence as set forth in SEQ ID NO: 92; and (c) a CDR3 having an amino acid sequence as set forth in SEQ ID NO: 97; and a light chain variable region including (a) a CDR1 having an amino acid sequence as set forth in SEQ ID NO: 83 (b) a CDR2 having an amino acid sequence as set forth in SEQ ID NO: 45; and (c) a CDR3 having an amino acid sequence as set forth in SEQ ID NO: 105.

In another aspect the present invention is directed to an anti-CD98 antibody, or antigen-binding portion thereof, having a heavy chain variable region including an amino acid sequence as set forth in SEQ ID NO: 99, and a light chain variable region including an amino acid sequence set forth in SEQ ID NO: 94.

In another aspect the present invention is directed to an anti-CD98 antibody, or antigen-binding portion thereof, having a heavy chain variable domain region including (a) a CDR1 having an amino acid sequence as set forth in SEQ ID NO: 79; (b) a CDR2 having an amino acid sequence as set forth in SEQ ID NO: 100; and (c) a CDR3 having an amino acid sequence as set forth in SEQ ID NO: 97; and a light chain variable region including (a) a CDR1 having an amino acid sequence as set forth in SEQ ID NO: 83 (b) a CDR2 having an amino acid sequence as set forth in SEQ ID NO: 45; and (c) a CDR3 having an amino acid sequence as set forth in SEQ ID NO: 95.

In another aspect the present invention is directed to an anti-CD98 antibody, or antigen-binding portion thereof, having a heavy chain variable region including an amino acid sequence as set forth in SEQ ID NO: 99, and a light chain variable region including an amino acid sequence set forth in SEQ ID NO: 101.

In another aspect the present invention is directed to an anti-CD98 antibody, or antigen-binding portion thereof, having a heavy chain variable domain region including (a) a CDR1 having an amino acid sequence as set forth in SEQ ID NO: 79; (b) a CDR2 having an amino acid sequence as set forth in SEQ ID NO: 100; and (c) a CDR3 having an amino acid sequence as set forth in SEQ ID NO: 97; and a light chain variable region including (a) a CDR1 having an amino acid sequence as set forth in SEQ ID NO: 83 (b) a CDR2 having an amino acid sequence as set forth in SEQ ID NO: 45; and (c) a CDR3 having an amino acid sequence as set forth in SEQ ID NO: 102.

In another aspect the present invention is directed to an anti-CD98 antibody, or antigen-binding portion thereof, having a heavy chain variable region including an amino acid sequence as set forth in SEQ ID NO: 103, and a light chain variable region including an amino acid sequence set forth in SEQ ID NO: 101.

In another aspect, the present invention is directed to an anti-CD98 antibody, or antigen-binding portion thereof, having a heavy chain variable domain region including (a) a CDR1 having an amino acid sequence as set forth in SEQ ID NO: 79; (b) a CDR2 having an amino acid sequence as set forth in SEQ ID NO: 104; and (c) a CDR3 having an amino acid sequence as set forth in SEQ ID NO: 97; and a light chain variable region including (a) a CDR1 having an amino acid sequence as set forth in SEQ ID NO: 83 (b) a CDR2 having an amino acid sequence as set forth in SEQ ID NO: 45; and (c) a CDR3 having an amino acid sequence as set forth in SEQ ID NO: 102.

In another aspect, the present invention is directed to an anti-CD98 antibody, or antigen-binding portion thereof, having a heavy chain variable region including an amino acid sequence as set forth in SEQ ID NO: 103, and a light chain variable region including an amino acid sequence set forth in SEQ ID NO: 98.

In another aspect the present invention is directed to an anti-CD98 antibody, or antigen-binding portion thereof, having a heavy chain variable domain region including (a) a CDR1 having an amino acid sequence as set forth in SEQ ID NO: 79; (b) a CDR2 having an amino acid sequence as set forth in SEQ ID NO: 104; and (c) a CDR3 having an amino acid sequence as set forth in SEQ ID NO: 97; and a light chain variable region including (a) a CDR1 having an amino acid sequence as set forth in SEQ ID NO: 83 (b) a CDR2 having an amino acid sequence as set forth in SEQ ID NO: 45; and (c) a CDR3 having an amino acid sequence as set forth in SEQ ID NO: 105.

Humanized antibodies huAb3v1, huAb3v2, huAb15v1, huAb15v2, and huAb15v6 were re-engineered using alternative framework regions in order to improve conjugation efficiency (as described in Example 14, below). Ten humanized framework engineered antibodies that maintained binding to human CD98 are listed in Table 18 as huAb101, huAb102, huAb103, huAb104, huAb105, huAb106, huAb107, huAb105, huAb109, and huAb110. The CDR, VH, and VL amino acid sequences of huAb101, huAb102, huAb103, huAb104, huAb105, huAb106, huAb107, huAb105, huAb109, and huAb1 10 mAbs are listed in Table 19.

The heavy chain variable sequence of huAb101 is provided in SEQ ID NO: 106 with CDR1, CDR2, and CDR3 sequences described in SEQ ID NOs: 16, 87, and 17, respectively. The light chain variable sequence of huAb101 is provided in SEQ ID NO: 107 with CDR1, CDR2, and CDR3 sequences described in SEQ ID NOs: 13, 7, and 19, respectively.

The heavy chain variable sequence of huAb102 is provided in SEQ ID NO: 108 with CDR1, CDR2, and CDR3 sequences described in SEQ ID NOs: 16, 87, and 17, respectively. The light chain variable sequence of huAb102 is provided in SEQ ID NO: 107 with CDR1, CDR2, and CDR3 sequences described in SEQ ID NOs: 13, 7, and 19, respectively.

The heavy chain variable sequence of huAb103 is provided in SEQ ID NO: 109 with CDR1, CDR2, and CDR3 sequences described in SEQ ID NOs: 16, 90, and 17, respectively. The light chain variable sequence of huAb103 is provided in SEQ ID NO: 107 with CDR1, CDR2, and CDR3 sequences described in SEQ ID NOs: 13, 7, and 19, respectively.

The heavy chain variable sequence of huAb104 is provided in SEQ ID NO: 110 with CDR1. CDR2, and CDR3 sequences described in SEQ ID NOs: 16, 90, and 17, respectively. The light chain variable sequence of huAb104 is provided in SEQ ID NO: 107 with CDR1, CDR2, and CDR3 sequences described in SEQ ID NOs: 13, 7, and 19, respectively.

The heavy chain variable sequence of huAb105 is provided in SEQ ID NO: 111 with CDR1. CDR2, and CDR3 sequences described in SEQ ID NOs: 79, 92, and 93, respectively. The light chain variable sequence of huAb105 is provided in SEQ ID NO: 112 with CDR1, CDR2, and CDR3 sequences described in SEQ ID NOs: 83, 45, and 95, respectively.

The heavy chain variable sequence of huAb106 is provided in SEQ ID NO: 113 with CDR1, CDR2, and CDR3 sequences described in SEQ ID NOs: 79, 92, and 93, respectively. The light chain variable sequence of huAb106 is provided in SEQ ID NO: 112 with CDR1, CDR2, and CDR3 sequences described in SEQ ID NOs: 83, 45, and 95, respectively.

The heavy chain variable sequence of huAb107 is provided in SEQ ID NO: 114 with CDR1, CDR2, and CDR3 sequences described in SEQ ID NOs: 79, 92, and 97, respectively. The light chain variable sequence of huAb107 is provided in SEQ ID NO: 112 with CDR1, CDR2, and CDR3 sequences described in SEQ ID NOs: 83, 45, and 95, respectively.

The heavy chain variable sequence of huAb108 is provided in SEQ ID NO: 115 with CDR1. CDR2, and CDR3 sequences described in SEQ ID NOs: 79, 92, and 97, respectively. The light chain variable sequence of huAb108 is provided in SEQ ID NO: 112 with CDR1, CDR2, and CDR3 sequences described in SEQ ID NOs: 83, 45, and 95, respectively.

The heavy chain variable sequence of huAb109 is provided in SEQ ID NO: 116 with CDR1. CDR2, and CDR3 sequences described in SEQ ID NOs: 79, 104, and 97, respectively. The light chain variable sequence of huAb109 is provided in SEQ ID NO: 117 with CDR1, CDR2, and CDR3 sequences described in SEQ ID NOs: 83, 45, and 102, respectively.

The heavy chain variable sequence of huAb110 is provided in SEQ ID NO: 118 with CDR1, CDR2, and CDR3 sequences described in SEQ ID NOs: 79, 104, and 97, respectively. The light chain variable sequence of huAb110 is provided in SEQ ID NO: 117 with CDR1, CDR2, and CDR3 sequences described in SEQ ID NOs: 83, 45, and 102, respectively.

Thus, in one aspect the present invention provides antibodies comprising variable and/or CDR sequences from a humanized antibody derived from chAb3 or chAb1S. In one embodiment, the invention features anti-CD98 antibodies which are derived from Ab3 have improved characteristics, e.g., improved binding affinity to isolated CD98 protein and improved binding to CD98 expressing cells, as described in the Examples below. Collectively these novel antibodies are referred to herein as “chAb3 variant antibodies” or “chAb1S variant antibodies.” Generally, the chAb3 variant antibodies retain the same epitope specificity as chAb3, and the chAb1S variant antibodies retain the same epitope specificity as chAb1S. In various embodiments, anti-CD98 antibodies, or antigen binding fragments thereof, of the invention are capable of modulating a biological function of CD98.

Thus, in one aspect, the present invention is directed to a humanized anti-CD98 antibody, or antigen-binding portion thereof, having a heavy chain variable region including an amino acid sequence set forth in SEQ ID NOs: 106, 108, 109, 110, 111, 113, 114, 115, 116, or 118; and/or a light chain variable region including an amino acid sequence set forth in SEQ ID NOs: 107, 112, or 117.

In another aspect, the present invention is directed to a humanized anti-CD98 antibody, or antigen binding portion thereof, of the invention comprises a heavy chain variable region comprising a CDR1 domain comprising an amino acid sequence as set forth in SEQ ID NO: 16 or 79; a CDR2 domain comprising an amino acid sequence as set forth in SEQ ID NO: 87, 90, 92, or 104; and a CDR3 domain comprising an amino acid sequence as set forth in SEQ ID NO: 17, 93, or 97; and a light chain variable region comprising a CDR1 domain comprising an amino acid sequence as set forth in SEQ ID NO: 13 or 83; a CDR2 domain comprising an amino acid sequence as set forth in SEQ ID NO: 7 or 45; and a CDR3 domain comprising an amino acid sequence as set forth in SEQ ID NO: 19, 95 or 102.

In another aspect, the present invention is directed to a humanized anti-CD98 antibody, or antigen-binding portion thereof, having a heavy chain variable region including an amino acid sequence as set forth in SEQ ID NO: 106 or 108, and a light chain variable region including an amino acid sequence set forth in SEQ ID NO: 107.

In another aspect, the present invention is directed to a humanized anti-CD98 antibody, or antigen-binding portion thereof, having a heavy chain variable domain region including (a) a CDR1 having an amino acid sequence as set forth in SEQ ID NO: 16; (b) a CDR2 having an amino acid sequence as set forth in SEQ ID NO: 87; and (c) a CDR3 having an amino acid sequence as set forth in SEQ ID NO: 17; and a light chain variable region including (a) a CDR1 having an amino acid sequence as set forth in SEQ ID NO: 13; (b) a CDR2 having an amino acid sequence as set forth in SEQ ID NO: 7; and (c) a CDR3 having an amino acid sequence as set forth in SEQ ID NO: 19.

In another aspect, the present invention is directed to a humanized anti-CD98 antibody, or antigen-binding portion thereof, having a heavy chain variable region including an amino acid sequence as set forth in SEQ ID NO: 109 or 110, and a light chain variable region including an amino acid sequence set forth in SEQ ID NO: 107.

In another aspect, the present invention is directed to a humanized anti-CD98 antibody, or antigen-binding portion thereof, having a heavy chain variable domain region including (a) a CDR1 having an amino acid sequence as set forth in SEQ ID NO: 16; (b) a CDR2 having an amino acid sequence as set forth in SEQ ID NO: 90; and (c) a CDR3 having an amino acid sequence as set forth in SEQ ID NO: 17; and a light chain variable region including (a) a CDR1 having an amino acid sequence as set forth in SEQ ID NO: 13; (b) a CDR2 having an amino acid sequence as set forth in SEQ ID NO: 7; and (c) a CDR3 having an amino acid sequence as set forth in SEQ ID NO: 19.

In another aspect, the present invention is directed to a humanized anti-CD98 antibody, or antigen-binding portion thereof, having a heavy chain variable region including an amino acid sequence as set forth in SEQ ID NO: 111 or 113, and a light chain variable region including an amino acid sequence set forth in SEQ ID NO: 112.

In another aspect, the present invention is directed to a humanized anti-CD98 antibody, or antigen-binding portion thereof, having a heavy chain variable domain region including (a) a CDR1 having an amino acid sequence as set forth in SEQ ID NO: 79; (b) a CDR2 having an amino acid sequence as set forth in SEQ ID NO: 92; and (c) a CDR3 having an amino acid sequence as set forth in SEQ ID NO: 93; and a light chain variable region including (a) a CDR1 having an amino acid sequence as set forth in SEQ ID NO: 83; (b) a CDR2 having an amino acid sequence as set forth in SEQ ID NO: 45; and (c) a CDR3 having an amino acid sequence as set forth in SEQ ID NO: 95.

In another aspect, the present invention is directed to a humanized anti-CD98 antibody, or antigen-binding portion thereof, having a heavy chain variable region including an amino acid sequence as set forth in SEQ ID NO: 114 or 115, and a light chain variable region including an amino acid sequence set forth in SEQ ID NO: 112.

In another aspect, the present invention is directed to a humanized anti-CD98 antibody, or antigen-binding portion thereof, having a heavy chain variable domain region including (a) a CDR1 having an amino acid sequence as set forth in SEQ ID NO: 79; (b) a CDR2 having an amino acid sequence as set forth in SEQ ID NO: 92; and (c) a CDR3 having an amino acid sequence as set forth in SEQ ID NO: 97; and a light chain variable region including (a) a CDR1 having an amino acid sequence as set forth in SEQ ID NO: 83; (b) a CDR2 having an amino acid sequence as set forth in SEQ ID NO: 45; and (c) a CDR3 having an amino acid sequence as set forth in SEQ ID NO: 95.

In another aspect, the present invention is directed to a humanized anti-CD98 antibody, or antigen-binding portion thereof, having a heavy chain variable region including an amino acid sequence as set forth in SEQ ID NO: 116 or 118, and a light chain variable region including an amino acid sequence set forth in SEQ ID NO: 117.

In another aspect, the present invention is directed to a humanized anti-CD98 antibody, or antigen-binding portion thereof, having a heavy chain variable domain region including (a) a CDR1 having an amino acid sequence as set forth in SEQ ID NO: 79; (b) a CDR2 having an amino acid sequence as set forth in SEQ ID NO: 104; and (c) a CDR3 having an amino acid sequence as set forth in SEQ ID NO: 97; and a light chain variable region including (a) a CDR1 having an amino acid sequence as set forth in SEQ ID NO: 83; (b) a CDR2 having an amino acid sequence as set forth in SEQ ID NO: 45; and (c) a CDR3 having an amino acid sequence as set forth in SEQ ID NO: 102.

Of the ten humanized antibodies huAb101, huAb102, huAb103, huAb104, huAb105, huAb106, huAb107, huAb105, huAb109, and huAb110, four (huAb102, huAb104, huAb108, and hAb110) were selected to be conjugated to various Bcl-xL inhibitors, as described in Example 16. In vitro potencies of these conjugates are listed in Table 23.

In another aspect, the invention provides an anti-CD98 antibody, or antigen binding fragment thereof, that specifically competes with an anti-CD98 antibody, or fragment thereof as described herein, wherein said competition can be detected in a competitive binding assay using said antibody, the human CD98 polypeptide, and the anti-CD98 antibody or fragment thereof. In particular embodiments, the competing antibody, or antigen binding portion thereof is an antibody, or antigen binding portion thereof that competes with huAb102, huAb104, huAb108, and hAb110.

In one embodiment, the anti-CD98 antibodies, or antigen binding portions thereof of the invention bind to CD98 (SEQ ID NO: 124) with a dissociation constant (K_{D}) of about 1×10⁻⁶ M or less, as determined by surface plasmon resonance. Alternatively, the antibodies, or antigen binding portions thereof, bind to CD98 (SEQ ID NO: 124) with a K_{D} of between about 1×10⁻⁶ M and about 1×10⁻¹¹ M, as determined by surface plasmon resonance. In a further alternative, antibodies, or antigen binding portions thereof, bind to CD98 (SEQ ID NO: 124) with a K_{D} of between about 1×10⁻⁶ M and about 1×10⁻¹⁰ M, as determined by surface plasmon resonance. Alternatively, antibodies, or antigen binding portions thereof, of the invention bind to CD98 (SEQ ID NO: 124) with a K_{D} of between about 1×10⁻⁶ M and about 5×10⁻¹⁰ M; a K_{D} of between about 1×10⁻⁶ M and about 1×10⁻⁹ M; a K_{d} of between about 1×10⁻⁶ M and about 5×10⁻⁹M; a K_{D} of between about 1×10⁻⁶ M and about 1×10⁻⁸ M; a K_{D} of between about 1×10⁻⁶ M and about 5×10⁻⁸ M; a K_{D} of between about 5.9×10⁻⁷ M and about 1.7×10⁻⁹ M; a K_{D} of between about 5.9×10⁻⁷ M and about 2.2×10⁻⁷ M, as determined by surface plasmon resonance.

It should be noted that anti-CD98 antibodies, or antigen binding portions thereof, having combinations of the aforementioned characteristics are also considered to be embodiments of the invention. For example, antibodies of the invention may bind to CD98 (SEQ ID NO: 124) with a dissociation constant (K_{D}) of about 1×10⁻⁶ M or less, as determined by surface plasmon resonance.

In one embodiment, the invention features an anti-CD98 antibody, or antigen binding portion thereof, which is the antibody huAb102. The huAb102 antibody comprises a heavy chain variable region comprising a CDR3 domain comprising the amino acid sequence of SEQ ID NO: 16, a CDR2 domain comprising the amino acid sequence of SEQ ID NO: 87, and a CDR1 domain comprising the amino acid sequence of SEQ ID NO: 17, and a light chain variable region comprising a CDR3 domain comprising the amino acid sequence of SEQ ID NO: 13, a CDR2 domain comprising the amino acid sequence of SEQ ID NO: 7, and a CDR1 domain comprising the amino acid sequence of SEQ ID NO: 19. In further embodiments, the invention provides an antibody having a heavy chain variable region comprising the amino acid sequence of SEQ ID NO: 108 and a light chain variable region comprising the amino acid sequence of SEQ ID NO: 107.

In one embodiment, the invention features an anti-CD98 antibody, or antigen binding portion thereof, which is the antibody huAb104. The huAb104 antibody comprises a heavy chain variable region comprising a CDR3 domain comprising the amino acid sequence of SEQ ID NO: 16, a CDR2 domain comprising the amino acid sequence of SEQ ID NO: 90, and a CDR1 domain comprising the amino acid sequence of SEQ ID NO: 17, and a light chain variable region comprising a CDR3 domain comprising the amino acid sequence of SEQ ID NO: 13, a CDR2 domain comprising the amino acid sequence of SEQ ID NO: 7, and a CDR1 domain comprising the amino acid sequence of SEQ ID NO: 19. In further embodiments, the invention provides an antibody having a heavy chain variable region comprising the amino acid sequence of SEQ ID NO: 110 and a tight chain variable region comprising the amino acid sequence of SEQ ID NO: 107.

In one embodiment, the invention features an anti-CD98 antibody, or antigen binding portion thereof, which is the antibody huAb108. The huAb108 antibody comprises a heavy chain variable region comprising a CDR3 domain comprising the amino acid sequence of SEQ ID NO: 79, a CDR2 domain comprising the amino acid sequence of SEQ ID NO: 92, and a CDR1 domain comprising the amino acid sequence of SEQ ID NO: 97, and a tight chain variable region comprising a CDR3 domain comprising the amino acid sequence of SEQ ID NO: 83, a CDR2 domain comprising the amino acid sequence of SEQ ID NO: 45, and a CDR1 domain comprising the amino acid sequence of SEQ ID NO: 95. In further embodiments, the invention provides an antibody having a heavy chain variable region comprising the amino acid sequence of SEQ ID NO: 115 and a tight chain variable region comprising the amino acid sequence of SEQ ID NO: 112.

In one embodiment, the invention features an anti-CD98 antibody, or antigen binding portion thereof, which is the antibody huAb110. The huAb110 antibody comprises a heavy chain variable region comprising a CDR3 domain comprising the amino acid sequence of SEQ ID NO: 79, a CDR2 domain comprising the amino acid sequence of SEQ ID NO: 104, and a CDR1 domain comprising the amino acid sequence of SEQ ID NO: 97, and a tight chain variable region comprising a CDR3 domain comprising the amino acid sequence of SEQ ID NO: 83, a CDR2 domain comprising the amino acid sequence of SEQ ID NO: 45, and a CDR1 domain comprising the amino acid sequence of SEQ ID NO: 102. In further embodiments, the invention provides an antibody having a heavy chain variable region comprising the amino acid sequence of SEQ ID NO: 118 and a tight chain variable region comprising the amino acid sequence of SEQ ID NO: 117.

In one embodiment, the anti-CD98 antibody, or antigen binding portion thereof, comprises a heavy chain variable region comprising an amino acid sequence selected from the group consisting of 106, 108, 109, 110, 111, 113, 114, 115, 116, and 118; and a tight chain variable region comprising an amino acid sequence selected from the group consisting of 107, 112, and 117.

In a further embodiment, the anti-CD98 antibody, or antigen binding portion thereof, of the invention comprises a heavy chain variable region comprising a CDR3 domain comprising an amino acid sequence as set forth in SEQ ID NO: 17, 93, or 97; a CDR2 domain comprising an amino acid sequence as set forth in SEQ ID NO: 87, 90, 92, or 104; and a CDR1 domain comprising an amino acid sequence as set forth in SEQ ID NO: 16, or 79; and a tight chain variable region comprising a CDR3 domain comprising an amino acid sequence as set forth in SEQ ID NO: 19, 95, or 102; a CDR2 domain comprising an amino acid sequence as set forth in SEQ ID NO: 7 or 45; and a CDR1 domain comprising an amino acid sequence as set forth in SEQ ID NO: 13 or 83.

The foregoing anti-CD98 antibody CDR sequences establish a novel family of CD98 binding proteins, isolated in accordance with this invention, and comprising antigen binding polypeptides that include the CDR sequences listed in Tables 6, 7, 15, and 19, as well as the Sequence Summary.

Anti-CD98 antibodies provided herein may comprise a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on the antibodies described herein (e.g., huAb102, huAb104, huAb105, or huAb110), or conservative modifications thereof, and wherein the antibodies retain the desired functional properties of the anti-CD98 antibodies described herein. Accordingly, the anti-CD98 antibody, or antigen binding portion thereof, may comprise a heavy chain variable region comprising CDR1, CDR2, and CDR3 sequences and a light chain variable region comprising CDR1, CDR2, and CDR3 sequences, wherein: (a) the heavy chain variable region CDR3 sequence comprises SEQ ID NO: 17 or 97, and conservative modifications thereof, e.g., 1, 2, 3, 4, 5, 1-2, 1-3, 1-4 or 1-5 conservative amino acid substitutions; (b) the light chain variable region CDR3 sequence comprises SEQ ID NO: 19, 95, or 102, and conservative modifications thereof, e.g., 1, 2, 3, 4, 5, 1-2, 1-3, 1-4 or 1-5 conservative amino acid substitutions; (c) the antibody specifically binds to CD98, and (d) the antibody exhibits 1, 2, 3, 4, 5, 6, or all of the following functional properties described herein, e.g., binding to human CD98. In a one embodiment, the heavy chain variable region CDR2 sequence comprises SEQ ID NO: 87, 90, 92, or 104, and conservative modifications thereof, e.g., 1, 2, 3, 4, 5, 1-2, 1-3, 1-4 or 1-5 conservative amino acid substitutions: and the light chain variable region CDR2 sequence comprises SEQ ID NO: 7 or 45, and conservative modifications thereof, e.g., 1, 2, 3, 4, 5, 1-2, 1-3, 1-4 or 1-5 conservative amino acid substitutions. In one embodiment, the heavy chain variable region CDR1 sequence comprises SEQ ID NO: 16 or 79, and conservative modifications thereof, e.g., 1, 2, 3, 4, 5, 1-2, 1-3, 1-4 or 1-5 conservative amino acid substitutions; and the light chain variable region CDR1 sequence comprises SEQ ID NO: 13 or 83, and conservative modifications thereof, e.g., 1, 2, 3, 4, 5, 1-2, 1-3, 1-4 or 1-5 conservative amino acid substitutions.

Conservative amino acid substitutions may also be made in portions of the antibodies other than, or in addition to, the CDRs. For example, conservative amino acid modifications may be made in a framework region or in the Fc region. A variable region or a heavy or light chain may comprise 1, 2, 3, 4, 5, 1-2, 1-3, 1-4, 1-5, 1-10, 1-15, 1-20, 1-25, or 1-50 conservative amino acid substitutions relative to the anti-CD98 antibody sequences provided herein. In certain embodiments, the anti-CD98 antibody comprises a combination of conservative and non-conservative amino acid modification. In one embodiment, the anti-CD98 antibody comprises a heavy chain variable region comprising SEQ ID NO: 108, 110, 115, or 118, and conservative modifications thereof, e.g., 1, 2, 3, 4, 5, 1-2, 1-3, 1-4 or 1-5 conservative amino acid substitutions; and a light chain variable region comprising SEQ ID NO: 107, 112, or 117, and conservative modifications thereof, e.g., 1, 2, 3, 4, 5, 1-2, 1-3, 1-4 or 1-5 conservative amino acid substitutions.

To generate and to select CDRs having preferred CD98 binding and/or neutralizing activity with respect to hCD98, standard methods known in the art for generating antibodies, or antigen binding portions thereof, and assessing the CD98 binding and/or neutralizing characteristics of those antibodies, or antigen binding portions thereof, may be used, including but not limited to those specifically described herein.

In certain embodiments, the antibody comprises a heavy chain constant region, such as an IgG1, IgG2, IgG3, IgG4, IgA, IgE, IgM, or IgD constant region. In certain embodiments, the anti-CD98 antibody, or antigen binding portion thereof, comprises a heavy chain immunoglobulin constant domain selected from the group consisting of a human IgG constant domain, a human IgM constant domain, a human IgE constant domain, and a human IgA constant domain. In further embodiments, the antibody, or antigen binding portion thereof, has an IgG1 heavy chain constant region, an IgG2 heavy chain constant region, an IgG3 constant region, or an IgG4 heavy chain constant region. Preferably, the heavy chain constant region is an IgG1 heavy chain constant region or an IgG4 heavy chain constant region. Furthermore, the antibody can comprise a light chain constant region, either a kappa light chain constant region or a lambda light chain constant region. Preferably, the antibody comprises a kappa light chain constant region. Alternatively, the antibody portion can be, for example, a Fab fragment or a single chain Fv fragment.

In certain embodiments, the anti-CD98 antibody binding portion is a Fab, a Fab″, a F(ab′)2, a Fv, a disulfide linked Fv, an scFv, a single domain antibody, or a diabody.

In certain embodiments, the anti-CD98 antibody, or antigen binding portion thereof, is a multispecific antibody, e.g. a bispecific antibody.

In certain embodiments, the anti-CD98 antibody, or antigen binding portion thereof, comprises a heavy chain constant region comprising the amino acid sequence set forth in SEQ ID NO: 108, 110, 115, or 118 and/or a light chain constant region comprising the amino acid sequence set forth in SEQ ID NO: 107, 112, or 117, e.g., huAb102, huAb104, huAB108, or huAb110.

Replacements of amino acid residues in the Fc portion to alter antibody effector function have been described (Winter, et al. U.S. Pat. Nos. 5,648,260 and 5,624,821, incorporated by reference herein). The Fc portion of an antibody mediates several important effector functions e.g, cytokine induction, ADCC, phagocytosis, complement dependent cytotoxicity (CDC) and half-life/clearance rate of antibody and antigen-antibody complexes. In some cases these effector functions are desirable for therapeutic antibody but in other cases might be unnecessary or even deleterious, depending on the therapeutic objectives. Certain human IgG isotypes, particularly IgG1 and IgG3, mediate ADCC and CDC via binding to FcγRs and complement C1q, respectively. Neonatal Fc receptors (FcRn) are the critical components determining the circulating half-life of antibodies. In still another embodiment at least one amino acid residue is replaced in the constant region of the antibody, for example the Fc region of the antibody, such that effector functions of the antibody are altered.

One embodiment of the invention includes a recombinant chimeric antigen receptor (CAR) comprising the binding regions of the antibodies described herein, e.g., the heavy and/or light chain CDRs of huAb102, huAb104, huAb108, or huAb110. A recombinant CAR, as described herein, may be used to redirect T cell specificity to an antigen in a human leukocyte antigen (HLA)-independent fashion Thus, CARs of the invention may be used in immunotherapy to help engineer a human subject's own immune cells to recognize and attack the subject's tumor (see, e.g., U.S. Pat. Nos. 6,410,319; 8,389,282; 8,822,647; 8,906,682; 8,911,993; 8,916,381; 8,975,071; and U.S. Patent Appln. Publ. No. US20140322275, each of which is incorporated by reference herein with respect to CAR technology). This type of immunotherapy is called adoptive cell transfer (ACT), and may be used to treat cancer in a subject in need thereof.

An anti-CD98 CAR of the invention preferably contains a extracellular antigen-binding domain specific for CD98, a transmembrane domain which is used to anchor the CAR into a T cell, and one or more intracellular signaling domains. In one embodiment of the invention, the CAR includes a transmembrane domain that comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154. In one embodiment of the invention, the CAR comprises a costimulatory domain, e.g., a costimulatory domain comprising a functional signaling domain of a protein selected from the group consisting of OX40, CD2, CD27, CD28, CD5, ICAM-1, LFA-1 (CD1 1a/CD18), ICOS (CD278), and 4-1BB (CD137). In certain embodiments of the invention, the CAR comprises an scFv comprising the CDR or variable regions described herein e.g., CDRs or variable regions from the huAb102, huAb104, huAb108, or huAb110 antibody, a transmembrane domain, a co-stimulatory domain (e.g., a functional signaling domain from CD28 or 4-1BB), and a signaling domain comprising a functional signaling domain from CD3 (e.g., CD3-zeta).

In certain embodiments, the invention includes a T cell comprising a CAR (also referred to as a CAR T cell) comprising antigen binding regions, e.g, CDRs, of the antibodies described herein or an scFv described herein.

In certain embodiments of the invention, the CAR comprises a variable heavy light chain comprising a CDR3 domain comprising the amino acid sequence set forth in SEQ ID NO: 19, a CDR2 domain comprising the amino acid sequence set forth in SEQ ID NO: 7, and a CDR1 domain comprising the amino acid sequence set forth in SEQ ID NO: 13; and a heavy chain variable region comprising a CDR3 domain comprising the amino acid sequence set forth in SEQ ID NO: 17, a CDR2 domain comprising the amino acid sequence set forth in SEQ ID NO: 87, and a CDR1 domain comprising the amino acid sequence set forth in SEQ ID NO: 16.

In certain embodiments of the invention, the CAR comprises a variable heavy light chain comprising a CDR3 domain comprising the amino acid sequence set forth in SEQ ID NO: 19, a CDR2 domain comprising the amino acid sequence set forth in SEQ ID NO: 7, and a CDR1 domain comprising the amino acid sequence set forth in SEQ ID NO: 13; and a heavy chain variable region comprising a CDR3 domain comprising the amino acid sequence set forth in SEQ ID NO: 17, a CDR2 domain comprising the amino acid sequence set forth in SEQ ID NO: 90, and a CDR1 domain comprising the amino acid sequence set forth in SEQ ID NO: 16.

In certain embodiments of the invention, the CAR comprises a variable heavy light chain comprising a CDR3 domain comprising the amino acid sequence set forth in SEQ ID NO: 95, a CDR2 domain comprising the amino acid sequence set forth in SEQ ID NO: 45, and a CDR1 domain comprising the amino acid sequence set forth in SEQ ID NO: 83: and a heavy chain variable region comprising a CDR3 domain comprising the amino acid sequence set forth in SEQ ID NO: 97, a CDR2 domain comprising the amino acid sequence set forth in SEQ ID NO: 92, and a CDR1 domain comprising the amino acid sequence set forth in SEQ ID NO: 79.

In certain embodiments of the invention, the CAR comprises a variable heavy light chain comprising a CDR3 domain comprising the amino acid sequence set forth in SEQ ID NO: 102, a CDR2 domain comprising the amino acid sequence set forth in SEQ ID NO: 45, and a CDR1 domain comprising the amino acid sequence set forth in SEQ ID NO: 83; and a heavy chain variable region comprising a CDR3 domain comprising the amino acid sequence set forth in SEQ ID NO: 97, a CDR2 domain comprising the amino acid sequence set forth in SEQ ID NO: 104, and a CDR1 domain comprising the amino acid sequence set forth in SEQ ID NO: 79.

One embodiment of the invention includes a labeled anti-CD98 antibody, or antibody portion thereof, where the antibody is derivatized or linked to one or more functional molecule(s) (e.g., another peptide or protein). For example, a labeled antibody can be derived by functionally linking an antibody or antibody portion of the invention (by chemical coupling, genetic fusion, noncovalent association or otherwise) to one or more other molecular entities, such as another antibody (e.g., a bispecific antibody or a diabody), a detectable agent, a pharmaceutical agent, a protein or peptide that can mediate the association of the antibody or antibody portion with another molecule (such as a streptavidin core region or a polyhistidine tag), and/or a cytotoxic or therapeutic agent selected from the group consisting of a mitotic inhibitor, an antitumor antibiotic, an immunomodulating agent, a vector for gene therapy, an alkylating agent, an antiangiogenic agent, an antimetabolite, a boron-containing agent, a chemoprotective agent a hormone, an antihormone agent a corticosteroid, a photoactive therapeutic agent an oligonucleotide, a radionuclide agent a topoisomerase inhibitor, a kinase inhibitor, a radiosensitizer, and a combination thereof.

Useful detectable agents with which an antibody, or antibody portion thereof, or ADC may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, glucose oxidase and the like. When an antibody is derivatized with a detectable enzyme, it is detected by adding additional reagents that the enzyme uses to produce a detectable reaction product. For example, when the detectable agent horseradish peroxidase is present the addition of hydrogen peroxide and diaminobenzidine leads to a colored reaction product, which is detectable. An antibody may also be derivatized with biotin, and detected through indirect measurement of avidin or streptavidin binding.

In one embodiment, the antibody or ADC of the invention is conjugated to an imaging agent. Examples of imaging agents that may be used in the compositions and methods described herein include, but are not limited to, a radiolabel (e.g., indium), an enzyme, a fluorescent label, a luminescent label, a bioluminescent label, a magnetic label, and biotin.

In one embodiment, the antibodies or ADCs are linked to a radiolabel, such as, but not limited to, indium (¹¹¹n). ¹¹¹Indium may be used to label the antibodies and ADCs described herein for use in identifying CD98 positive tumors. In a certain embodiment, anti-CD98 antibodies (or ADCs) described herein are labeled with ¹¹¹I via a bifunctional chelator which is a bifunctional cyclohexyl diethylenetriaminepentaacetic acid (DTPA) chelate (see U.S. Pat. No. 5,124,471; 5,434,287; and 5,286,850, each of which is incorporated herein by reference).

Another embodiment of the invention provides a glycosylated binding protein wherein the anti-CD98 antibody or antigen binding portion thereof comprises one or more carbohydrate residues. Nascent in vivo protein production may undergo further processing, known as post-translational modification. In particular, sugar (glycosyl) residues may be added enzymatically, a process known as glycosylation. The resulting proteins bearing covalently linked oligosaccharide side chains are known as glycosylated proteins or glycoproteins. Antibodies are glycoproteins with one or more carbohydrate residues in the Fc domain, as well as the variable domain. Carbohydrate residues in the Fc domain have important effect on the effector function of the Fc domain, with minimal effect on antigen binding or half-life of the antibody (R. Jefferis, Biotechnol. Prog. 21 (2005), pp. 11-16). In contrast, glycosylation of the variable domain may have an effect on the antigen binding activity of the antibody. Glycosylation in the variable domain may have a negative effect on antibody binding affinity, likely due to steric hindrance (Co, M. S., et al., Mol. Immunol. (1993) 30:1361-1367), or result in increased affinity for the antigen (Wallick. S. C., et al., Exp. Med (1988) 168:1099-1109; Wright, A, et al, EMBO J (1991) 10:2717-2723).

One aspect of the invention is directed to generating glycosylation site mutants in which the O- or N-linked glycosylation site of the binding protein has been mutated. One skilled in the art can generate such mutants using standard well-known technologies. Glycosylation site mutants that retain the biological activity, but have increased or decreased binding activity, are another object of the invention.

In still another embodiment, the glycosylation of the anti-CD98 antibody or antigen binding portion of the invention is modified. For example, an aglycosylated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region glycosylation sites to thereby eliminate glycosylation at that site. Such aglycosylation may increase the affinity of the antibody for antigen. Such an approach is described in further detail in PCT Publication WO2003016466A2, and U.S. Pat. Nos. 5,714,350 and 6,350,861, each of which is incorporated herein by reference in its entirety.

Additionally or alternatively, a modified anti-CD98 antibody of the invention can be made that has an altered type of glycosylation, such as a hypofucosylated antibody having reduced amounts of fucosyl residues or an antibody having increased bisecting GlcNAc structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. See, for example. Shields, R. L. et al. (2002) J. Biol. Chem. 277:26733-26740; Umana et al. (1999) Nat. Biotech. 17:176-1, as well as, European Patent No: EP 1,176,195; PCT Publications WO 03/035835; WO 99/54342 80, each of which is incorporated herein by reference in its entirety.

Protein glycosylation depends on the amino acid sequence of the protein of interest, as well as the host cell in which the protein is expressed. Different organisms may produce different glycosylation enzymes (e.g., glycosyltransferases and glycosidases), and have different substrates (nucleotide sugars) available. Due to such factors, protein glycosylation pattern, and composition of glycosyl residues, may differ depending on the host system in which the particular protein is expressed. Glycosyl residues useful in the invention may include, but are not limited to, glucose, galactose, mannose, fucose, n-acetylglucosamine and sialic acid. Preferably the glycosylated binding protein comprises glycosyl residues such that the glycosylation pattern is human.

Differing protein glycosylation may result in differing protein characteristics. For instance, the efficacy of a therapeutic protein produced in a microorganism host, such as yeast, and glycosylated utilizing the yeast endogenous pathway may be reduced compared to that of the same protein expressed in a mammalian cell, such as a CHO cell line. Such glycoproteins may also be immunogenic in humans and show reduced half-life in v/m after administration. Specific receptors in humans and other animals may recognize specific glycosyl residues and promote the rapid clearance of the protein from the bloodstream. Other adverse effects may include changes in protein folding, solubility, susceptibility to proteases, trafficking, transport, compartmentalization, secretion, recognition by other proteins or factors, antigenicity, or allergenicity. Accordingly, a practitioner may prefer a therapeutic protein with a specific composition and pattern of glycosylation, for example glycosylation composition and pattern identical, or at least similar, to that produced in human cells or in the species-specific cells of the intended subject animal.

Expressing glycosylated proteins different from that of a host cell may be achieved by genetically modifying the host cell to express heterologous glycosylation enzymes. Using recombinant techniques, a practitioner may generate antibodies or antigen binding portions thereof exhibiting human protein glycosylation. For example, yeast strains have been genetically modified to express non-naturally occurring glycosylation enzymes such that glycosylated proteins (glycoproteins) produced in these yeast strains exhibit protein glycosylation identical to that of animal cells, especially human cells (U S, patent Publication Nos. 20040018590 and 20020137134 and PCT publication WO2005100584 A2).

Antibodies may be produced by any of a number of techniques. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation. DEAE-dextran transfection and the like. Although it is possible to express antibodies in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Preferred mammalian host cells for expressing the recombinant antibodies of the invention include Chinese Hamster Ovary (CHO cells) (including dhfr-CHO cells, described in Urlaub and Chasin, (1980) Proc. Natl. Acad Sci. USA 77:4216-4220, used with a DHFR selectable marker, e.g., as described in R. J. Kaufman and P. A. Sharp (1982) Mol. Biol. 159:601-621), NS0 myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure are within the scope of the invention. For example, it may be desirable to transfect a host cell with DNA encoding functional fragments of either the light chain and/or the heavy chain of an antibody of this invention. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than the antigens of interest by crosslinking an antibody of the invention to a second antibody by standard chemical crosslinking methods.

In a preferred system for recombinant expression of an antibody, or antigen binding portion thereof, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. Still further the invention provides a method of synthesizing a recombinant antibody of the invention by culturing a host cell in a suitable culture medium until a recombinant antibody is synthesized. Recombinant antibodies of the invention may be produced using nucleic acid molecules corresponding to the amino acid sequences disclosed herein. In one embodiment, the nucleic acid molecules set forth in SEQ ID NOs: 86 and/or 87 are used in the production of a recombinant antibody. The method can further comprise isolating the recombinant antibody from the culture medium.

The N- and C-termini of antibody polypeptide chains of the present invention may differ from the expected sequence due to commonly observed post-translational modifications. For example, C-terminal lysine residues are often missing from antibody heavy chains. Dick et al. (2008) Biotechnol. Bioeng. 100:1132. N-terminal glutamine residues, and to a lesser extent glutamate residues, are frequently converted to pyroglutamate residues on both light and heavy chains of therapeutic antibodies. Dick et al. (2007) Biotechnol. Bioeng. 97:544; Liu et al. (2011) JBC 28611211; Liu et al. (2011) J. Biol. Chem. 286:11211.

### III. Anti-CD98 Antibody Drug Conjugates (ADCs)

Anti-CD98 antibodies described herein may be conjugated to a drug moiety to form an anti-CD98 Antibody Drug Conjugate (ADC). Antibody-drug conjugates (ADCs) may increase the therapeutic efficacy of antibodies in treating disease, e.g., cancer, due to the ability of the ADC to selectively deliver one or mote drug moiety(s) to target tissues, such as a tumor-associated antigen. e.g., CD98 expressing tumors. Thus, in certain embodiments, the invention provides anti-CD98 ADCs for therapeutic use, e.g., treatment of cancer.

Anti-CD98 ADCs of the invention comprise an anti-CD98 antibody, i.e., an antibody that specifically binds to human CD98, linked to one or more drug moieties. The specificity of the ADC is defined by the specificity of the antibody, i.e., anti-CD98. In one embodiment, an anti-CD98 antibody is linked to one or more cytotoxic drug(s) which is delivered internally to a transformed cancer cell expressing CD98.

Examples of drugs that may be used in the anti-CD98 ADC of the invention are provided below, as are linkers that may be used to conjugate the antibody and the one or more drug(s). The terms “drug,” “agent,” and “drug moiety” are used interchangeably herein. The terms “linked” and “conjugated” are also used interchangeably herein and indicate that the antibody and moiety are covalently linked.

In some embodiments, the ADC has the following formula (formula I):

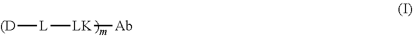

wherein Ab is the antibody, e.g., anti-CD98 antibody huAb102, huAb104, huAb105, or huAb110, and (D-L-LK) is a Drug-Linker-Covalent Linkage. The Drug-Linker moiety is made of L- which is a Linker, and -D, which is a drug moiety having, for example, cytostatic, cytotoxic, or otherwise therapeutic activity against a target cell, e.g., a cell expressing CD98; and m is an integer from 1 to 20. In some embodiments, m ranges from 1 to 8, 1 to 7, 1 to 6, 2 to 6, 1 to 5, 1 to 4, 2 to 4, or 1 to 3. The DAR of an ADC is equivalent to the “m” referred to in Formula I. In one embodiment, the ADC has a formula of Ab-(LK-L-D)ₘ, wherein Ab is an anti-CD98 antibody, e.g, huAb102, huAb104, huAb108, or huAb110, L is a linker, D is a drug, e.g., a Bcl-xL inhibitor. LK is a covalent linker, e.g. —S—, and m is 1 to 8 (or a DAR of 2-4). Additional details regarding drugs (D of Formula I) and linkers (L of Formula I) that may be used in the ADCs of the invention, as well as alternative ADC structures, are described below.

### III. A. Anti-CD98 ADCs: Bcl-xL Inhibitors, Linkers, Synthons, and Methods of Making Same

Dysregulated apoptotic pathways have also been implicated in the pathology of cancer. The implication that down-regulated apoptosis (and more particularly the Bcl-2 family of proteins) is involved in the onset of cancerous malignancy has revealed a novel way of targeting this still elusive disease. Research has shown, for example, the anti-apoptotic proteins, Bcl 2 and Bcl-xL, are over-expressed in many cancer cell types. See. Zhang, 2002. Nature Reviews/Drug Discovery 1:101; Kirkin et al., 2004. Biochimica Biophysica Acta 1644:229-249; and Amundson et al., 2000, Cancer Research 60:6101-6110. The effect of this deregulation is the survival of altered cells which would otherwise have undergone apoptosis in normal conditions. The repetition of these defects associated with unregulated proliferation is thought to be the starting point of cancerous evolution.

Aspects of the disclosure concern anti-hCD98 ADCs comprising an anti-hCD98 antibody conjugated to a drug via a linker, wherein the drug is a Bcl-xL inhibitor. In specific embodiments, the ADCs are compounds according to structural formula (I) below, or a pharmaceutically acceptable salt thereof, wherein Ab represents the anti-hCD98 antibody. D represents a Bcl-xL inhibitor drug (i.e., a compound of formula IIa or IIb as shown below), L represents a linker. LK represents a covalent linkage linking the linker (L) to the anti-hCD98 antibody (Ab) and m represents the number of D-L-LK units linked to the antibody, which is an integer ranging from 1 to 20. In certain embodiments, m is 2, 3 or 4.

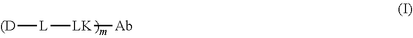

Specific embodiments of various Bcl-xL inhibitors per se, and various Bcl-xL inhibitors (D), linkers (L) and anti-CD98 antibodies (Ab) that can comprise the ADCs described herein, as well as the number of Bcl-xL inhibitors linked to the ADCs, are described in more detail below.

Examples of Bcl-xL inhibitors that may be used in the anti-CD98 ADC of the invention are provided below, as are linkers that may be used to conjugate the antibody and the one or more Bcl-xL inhibitor(s). The terms “linked” and “conjugated” are also used interchangeably herein and indicate that the antibody and moiety are covalently linked.

### III.A.1. Bcl-xL Inhibitors

The Bcl-xL inhibitors may be used as compounds or salts per se in the various methods described herein, or may be included as a component part of an ADC, e.g., as the drug (D) in formula (I).

Exemplary Bcl-xl inhibitors are described in International Publication No. WO 2016/094517, incorporated by reference in its entirety herein.

Specific embodiments of Bcl-xL inhibitors that may be used in unconjugated form, or that may be included as part of an ADC include compounds according to structural formula (IIa) or (IIb). In the present invention, when the Bcl-xL inhibitors are included as part of an ADC, # shown in formula (IIa) or (IIb) below represents a point of attachment to a linker, which indicates that they are represented in a monoradical form.

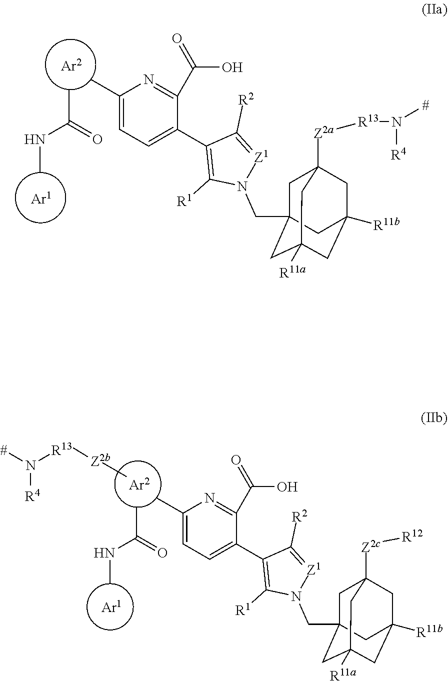

or salts thereof, wherein:

Ar¹ is selected from

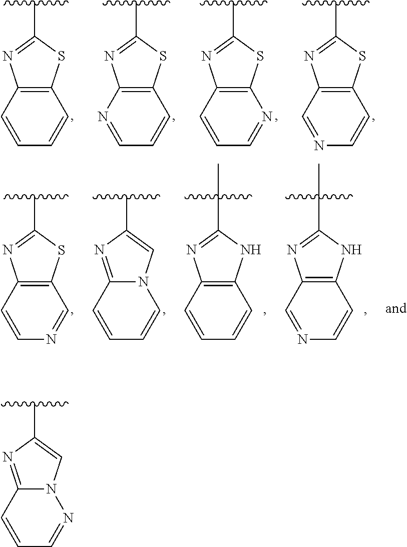

and is optionally substituted with one or more substituents independently selected from halo, hydroxy, nitro, lower alkyl, lower heteroalkyl, C₁₋₄alkoxy, amino, cyano and halomethyl;

Ar² is selected from

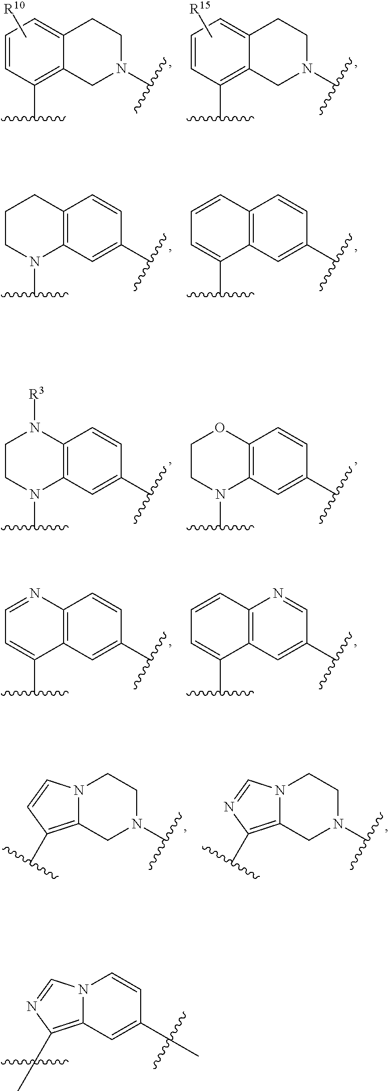

and is optionally substituted with one or more substituents independently selected from halo, hydroxy, nitro, lower alkyl, lower heteroalkyl, C₁₋₄alkoxy, amino, cyano and halomethyl, wherein the #—N(R⁴)—R¹³—Z^{2b}— substituent of formula (IIb) is attached to Ar² at any Ar² atom capable of being substituted;

Z¹ is selected from N, CH, C-halo and C—CN;

Z^{2a}, Z^{2b}, and Z^{2c} are each, independent from one another, selected from a bond, NR⁶, CR^{6a}R^{6b}, O, S, S(O), SO₂, NR⁶C(O), NR^{6a}C(O)NR^{6b}, and NR⁵C(O)O;

R¹ is selected from hydrogen, methyl, halo, halomethyl, ethyl and cyano;

R² is selected from hydrogen, methyl, halo, halomethyl and cyano;

R³ is selected from hydrogen, lower alkyl and lower heteroalkyl;

R⁴ is selected from hydrogen, lower alkyl, monocyclic cycloalkyl, monocyclic heterocyclyl, lower heteroalkyl or is taken together with an atom of R¹³ to form a cycloalkyl or heterocyclyl ring having between 3 and 7 ring atoms, wherein the lower alkyl, monocyclic cycloalkyl, monocyclic heterocyclyl, lower heteroalkyl are optionally substituted with one or more halo, cyano, C₁₋₄alkoxy, monocyclic cycloalkyl, monocyclic heterocyclyl, NHC(O)CR^{6a}R^{6b}, NHS(O)CR^{6a}R^{6b}. NHS(O)₂CR^{6a}R^{6b}, S(O)₂CR^{6a}R^{6b} or S(O)₂NH₂ groups;

R⁶, R^{6a} and R^{6b} are each, independent from one another, selected from hydrogen, lower alkyl, lower heteroalkyl, optionally substituted monocyclic cycloalklyl and monocyclic heterocyclyl, or are taken together with an atom from R¹³ to form a cycloalkyl or heterocyclyl ring having between 3 and 7 ring atoms;

R¹⁰ is selected from cyano, OR¹⁴, SR¹⁴, SOR¹⁴, SO₂R^{H}, SO₂NR^{14a}R^{14b}, NR^{14a}R^{14b}, NHC(O)R¹⁴ and NHSO₂R¹⁴;

R^{11a} and R^{11b} are each, independently of one another, selected from hydrogen, halo, methyl, ethyl, halomethyl, hydroxyl, methoxy, CN, and SCH₃;

R¹² is selected from hydrogen, halo, cyano, lower alkyl, lower heteroalkyl, cycloalkyl, or heterocyclyl, wherein the alkyl, heteroalkyl, cycloalkyl, or heterocyclyl are optionally substituted with one or more halo, cyano, C₁₋₄alkoxy, monocyclic cycloalkyl, monocyclic heterocyclyl, NHC(O)CR^{6a}R^{6b}, NHS(O)CR^{6a}R^{6b}, NHS(O)₂CR^{6a}R^{6b} or S(O)₂CR^{6a}R^{6b} groups;

R¹³ is selected from a bond, optionally substituted lower alkylene, optionally substituted lower heteroalkylene, optionally substituted cycloalkyl or optionally substituted heterocyclyl;

R¹⁴ is selected from hydrogen, optionally substituted lower alkyl and optionally substituted lower heteroalkyl;

R^{14a} and R^{14b} are each, independently of one another, selected from hydrogen, optionally substituted lower alkyl, optionally substituted lower heteroalkyl, or are taken together with the nitrogen atom to which they are bonded to form a monocyclic cycloalkyl or monocyclic heterocyclyl ring;

R¹⁵ is selected from hydrogen, halo, C₁₋₆ alkanyl, C₂₋₄ alkenyl, C₂₋₄ alkynyl, and C₁₋₄ haloalkyl and C₁₋₄ hydroxyalkyl, with the proviso that when R¹⁵ is present, R⁴ is not C₁₋₄ alkyl, C₂₋₄ alkenyl, C₂₋₄ alkynyl, C₁₋₄ haloalkyl or C₁₋₄ hydroxyalkyl, wherein the R⁴ C₁₋₆ alkanyl, C₂₋₄ alkenyl, C₂₋₄ alkynyl, C₁₋₄ haloalkyl and CM hydroxyalkyl are optionally substituted with one or more substituents independently selected from OCH), OCH₂CH₂OCH₃, and OCH₂CH₂NHCH₃; and

# represents a point of attachment to a linker or a hydrogen atom.

Specific embodiments of Bcl-xL inhibitors that may be used in unconjugated form, or that may be included as part of an ADC include compounds according to structural formula (IIa) or (IIb):

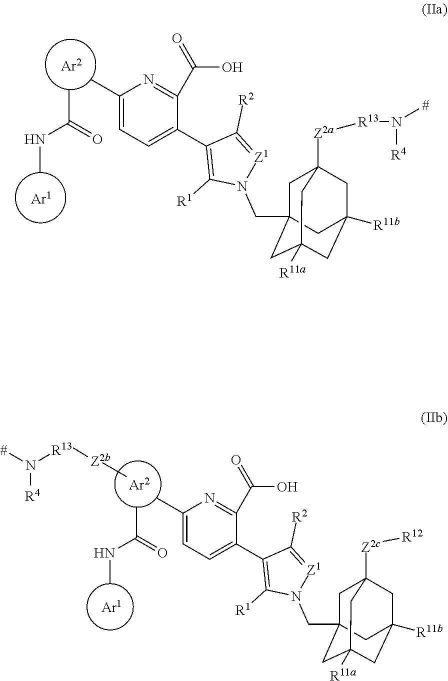

or salts thereof, wherein:

Ar¹ is selected from

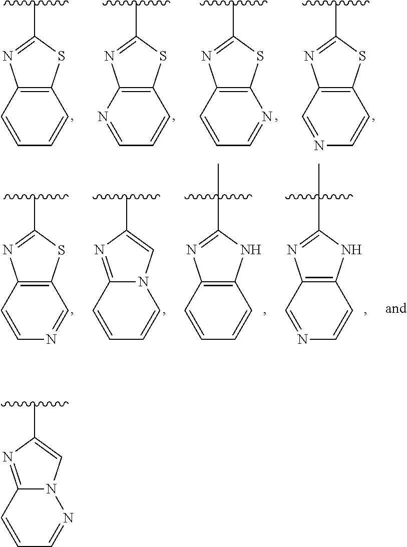

and is optionally substituted with one or more substituents independently selected from halo, hydroxy, nitro, lower alkyl, lower heteroalkyl, C₁₋₄alkoxy, amino, cyano and halomethyl;

Ar² is selected from

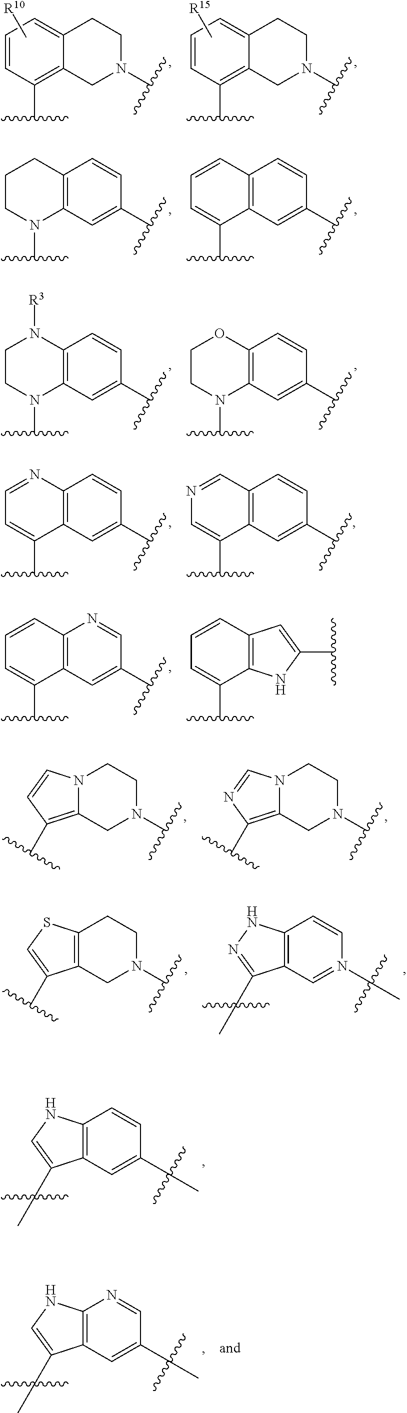

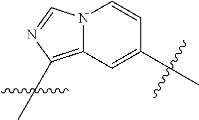 [SMILES: *c1ccn2cnc(*)c2c1]

and is optionally substituted with one or more substituents independently selected from halo, hydroxy, nitro, lower alkyl, lower heteroalkyl, C₁₋₄alkoxy, amino, cyano and halomethyl, wherein the #—N(R⁴)—R¹³—Z^{2b}— substituent of formula (IIb) is attached to Ar² at any Ar² atom capable of being substituted;

Z¹ is selected from N, CH, C-halo and C—CN;

Z^{2a}, Z^{2b}, and Z^{2c} are each, independent from one another, selected from a bond, NR⁶, CR^{6a}R^{6b}, O, S, S(O), S(O)₂, NR⁶C(O), NR^{6a}C(O)NR^{6b}, and NR⁶C(O)O;

R¹ is selected from hydrogen, methyl, halo, halomethyl, ethyl and cyano;

R² is selected from hydrogen, methyl, halo, halomethyl and cyano;

R³ is selected from hydrogen, lower alkyd and lower heteroalkyl;

R⁴ is selected from hydrogen, lower alkyl, monocyclic cycloalkyl, monocyclic heterocyclyl, and lower heteroalkyl or is taken together with an atom of R¹³ to form a cycloalkyl or heterocyclyl ring having between 3 and 7 ring atoms, wherein the lower alkyl, monocyclic cycloalkyl, monocyclic heterocyclyl, and lower heteroalkyl are optionally substituted with one or more halo, cyano, hydroxy, C₁₋₄alkoxy, monocyclic cycloalkyl, monocyclic heterocyclyl, C(O)NR^{6a}R^{6b}, S(O)₂NR^{6a}R^{6b}, NHC(O)CHR^{6a}R^{6b}, NHS(O)CHR^{6a}R^{6b}, NHS(O)CHR^{6a}R^{6b}, S(O)₂CHR^{6a}R^{6b} or S(O)₂NH₂ groups;

R⁶, R^{6a} and R^{6b} are each, independent from one another, selected from hydrogen, lower alkyl, lower heteroalkyl, optionally substituted monocyclic cycloalklyl and monocyclic heterocyclyl, or are taken together with an atom from R¹³ to form a cycloalkyl or heterocyclyl ring having between 3 and 7 ring atoms;

R¹⁰ is selected from cyano, OR¹⁴, SR¹⁴, SOR¹⁴, SO₂R¹⁴, SO₂NR^{14a}R^{14b}, NR^{14a}R^{14b}, NHC(O)R¹⁴ and NHSO₂R¹⁴;

R^{11a} and R^{11b} are each, independently of one another, selected from hydrogen, halo, methyl, ethyl, halomethyl, hydroxyl, methoxy, CN, and SCH₃;

R¹² is selected from hydrogen, halo, cyano, lower alkyl, lower heteroalkyl, cycloalkyl, and heterocyclyl, wherein the alkyl, heteroalkyl, cycloalkyl, and heterocyclyl are optionally substituted with one or more halo, cyano, C₁₋₄alkoxy, monocyclic cycloalkyl, monocyclic heterocyclyl, NHC(O)CHR^{6a}R^{6b}, NHS(O)CHR^{6a}R^{6b}, NHS(O)₂CHR^{6e}R^{6b} or S(O)₂CHR^{6a}, R^{6b} groups;

R¹³ is selected from a bond, optionally substituted lower alkylene, optionally substituted lower heteroalkylene, optionally substituted cycloalkyl or optionally substituted heterocyclyl:

R¹⁴ is selected from hydrogen, optionally substituted lower alkyl and optionally substituted lower heteroalkyl;

R^{14a} and R^{14b} are each, independently of one another, selected from hydrogen, optionally substituted lower alkyl, and optionally substituted lower heteroalkyl, or are taken together with the nitrogen atom to which they are bonded to form an optionally substituted monocyclic cycloalkyl or monocyclic heterocyclyl ring;

R¹⁵ is selected from hydrogen, halo, C₁₋₆ alkanyl, C₂₋₄ alkenyl, C₂₋₄ alkynyl, and C₁₋₄ haloalkyl and C₁₋₄ hydroxyalkyl, with the proviso that when R¹⁵ is present, R⁴ is not C₁₋₄ alkyl, C₂₋₄ alkenyl, C₂₋₄ alkynyl, C₁₋₄ haloalkyl C₁₋₄ hydroxyalkyl, wherein the R⁴ C₁₋₆ alkanyl, C₂₋₄ alkenyl, C₂₋₄ alkynyl, C₁₋₄ haloalkyl and C₁₋₄ hydroxyalkyl are optionally substituted with one or more substituents independently selected from OCH₃, OCH₂CH₂OCH₃, and OCH₂CH₂NHCH₃; and

# represents a point of attachment to a linker or a hydrogen atom.

Another embodiment of Bcl-xL inhibitors that may be used in unconjugatcd form, or that may be included as part of an ADC include compounds according to structural formula (IIa) or (IIb):

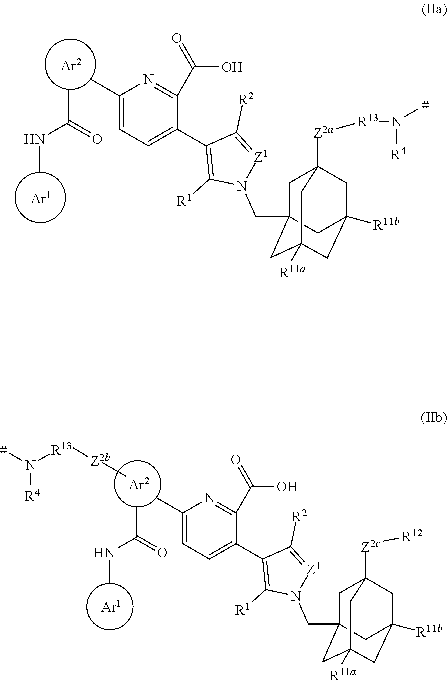

or salts thereof, wherein:

Ar¹ is selected from

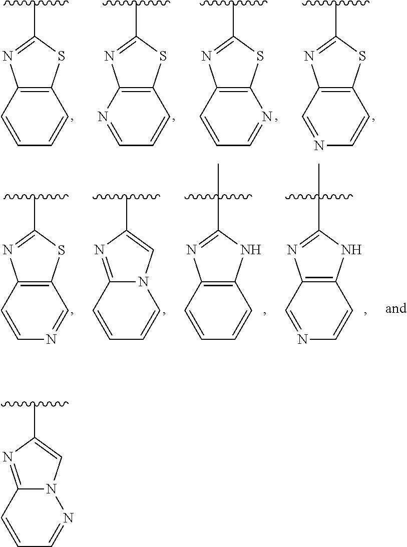

and is optionally substituted with one or more substituents independently selected from halo, hydroxy, nitro, lower alkyl, lower heteroalkyl, C₁₋₄ alkoxy, amino, cyano and halomethyl;
Ar² is selected from

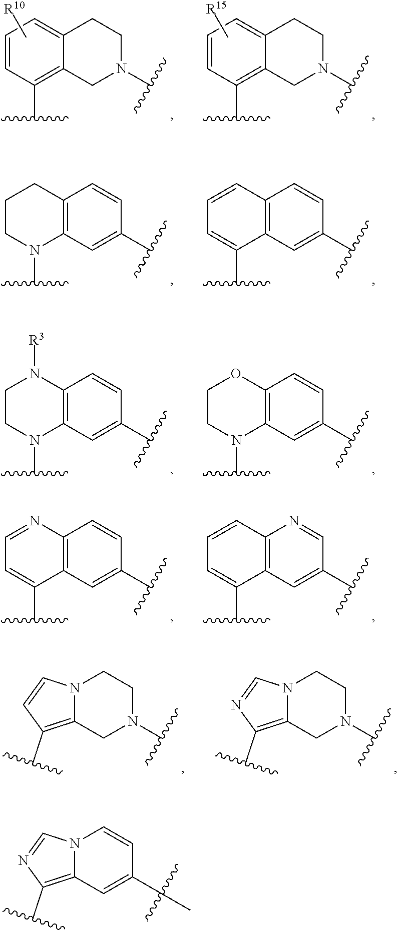

and is optionally substituted with one or more substituents independently selected from halo, hydroxy, nitro, lower alkyl, lower heteroalkyl, C₁₋₄ alkoxy, amino, cyano and halomethyl, wherein the #—N(R⁴)—R¹³—Z^{2b}— substituent of formula (IIb) is attached to Ar² at any Ar² atom capable of being substituted:
Z¹ is selected from N, CH, C-halo and C—CN;

Z^{2a}, Z^{2b}, and Z^{2c} are each, independent from one another, selected from a bond, NR⁶, CR^{6a}R^{6b}, O, S, S(O), SO₂, NR⁶C(O), NR^{6a}C(O)NR^{6b}, and NR⁶C(O)O;

R¹ is selected from hydrogen, methyl, halo, halomethyl, ethyl and cyano;

R² is selected from hydrogen, methyl, halo, halomethyl and cyano;

R³ is selected from hydrogen, lower alkyl and lower heteroalkyl;

R⁴ is selected from hydrogen, lower alkyl, monocyclic cycloalkyl, monocyclic heterocyclyl, lower heteroalkyl or is taken together with an atom of R¹³ to form a cycloalkyl or heterocyclyl ring having between 3 and 7 ring atoms, wherein the lower alkyl, monocyclic cycloalkyl, monocyclic heterocyclyl, lower heteroalkyl are optionally substituted with one or more halo, cyano, C₁₋₄alkoxy, monocyclic cycloalkyl, monocyclic heterocyclyl. NHC(O)CR^{6a}R^{6b}, NHS(O)CR^{6a}R^{6b}, NHS(O)₂CR^{6a}R^{6b}, S(O)₂CR^{6a}R^{6b} or S(O)₂NH₂ groups;

R⁶, R^{6a} and R^{6b} are each, independent from one another, selected from hydrogen, lower alkyl, lower heteroalkyl, optionally substituted monocyclic cycloalklyl and monocyclic heterocyclyl, or are taken together with an atom from R¹³ to form a cycloalkyl or heterocyclyl ring having between 3 and 7 ring atoms;

R¹⁰ is selected from cyano, OR¹⁴, SR¹⁴, SOR¹⁴, SOR¹⁴, SO₂NR^{14a}R^{14b}, NR^{14a}R^{14b}. NHC(O)R¹⁴ and NHSO₂R¹⁴;

R^{11a} and R^{11b} are each, independently of one another, selected from hydrogen, halo, methyl, ethyl, halomethyl, hydroxyl, methoxy, CN, and SCH₃;

R¹² is selected from hydrogen, halo, cyano, lower alkyl, lower heteroalkyl, cycloalkyl, or heterocyclyl, wherein the alkyl, heteroalkyl, cycloalkyl, or heterocyclyl are optionally substituted with one or more halo, cyano, C₁₋₄alkoxy, monocyclic cycloalkyl, monocyclic heterocyclyl, NHC(O)CR^{6a}R^{6b}, NHS(O)CR^{6a}R^{6b}, NHS(O)₂CR^{6a}R^{6b} or S(O)₂CR^{6a}R^{6b} groups;

R¹³ is selected from a bond, optionally substituted lower alkylene, optionally substituted lower heteroalkylene, optionally substituted cycloalkyl or optionally substituted heterocyclyl;

R¹⁴ is selected from hydrogen, optionally substituted lower alkyl and optionally substituted lower heteroalkyl;

R^{14e} and R^{14b} are each, independently of one another, selected from hydrogen, optionally substituted lower alkyl, optionally substituted lower heteroalkyl, or are taken together with the nitrogen atom to which they are bonded to form a monocyclic cycloalkyl or monocyclic heterocyclyl ring;

R¹⁵ is selected from hydrogen, halo. C₁₋₆ alkanyl, C₂₋₄ alkenyl, C₂₋₄ alkynyl, and C₁₋₄ haloalkyl and C₁₋₄ hydroxyalkyl, with the proviso that when R¹⁵ is present, R⁴ is not C₁₋₄ alkyl, C₂₋₄ alkenyl, C₂₋₄ alkynyl, C₁₋₄ haloalkyl or C₁₋₄ hydroxyalkyl, wherein the R⁴ C₁₋₆ alkanyl, C₂₋₄ alkenyl, C₂₋₄ alkynyl, C₁₋₄ haloalkyl and C₁₋₄ hydroxyalkyl are optionally substituted with one or more substituents independently selected from OCH₃, OCH₂CH₂OCH₃, and OCH₂CH₂NHCH₃; and

# represents a point of attachment to a linker or a hydrogen atom.

When a Bcl-xL inhibitor of structural formulae (IIa) and (IIb) is not a component of an ADC, # in formulae (IIa) and (IIb) represents the point of attachment to a hydrogen atom. When the Bcl-xL inhibitor is a component of an ADC. # in formulae (IIa) and (IIb) represents the point of attachment to a the linker. When a Bcl-xL inhibitor is a component of an ADC, the ADC may comprise one or more Bcl-xL inhibitors, which may be the same or different, but are typically the same.

In certain embodiments, Ar¹ of formula (IIa) or (IIb) is selected from

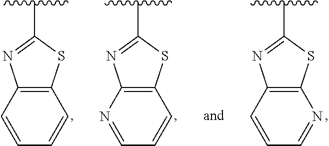

and is optionally substituted with one or more substituents independently selected from halo, cyano, methyl, and halomethyl. In particular embodiments, Ar¹ is

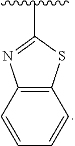 [SMILES: *c1nc2ccccc2s1]

In particular embodiments, Ar¹ is unsubstituted.

In all embodiments, the #—N(R⁴)—R¹³—Z^{2b}— substituent of formula (IIb) is attached to Ar² at any Ar² atom capable of being substituted.

In certain embodiments, Ar² of formula (IIa) or (IIb) is

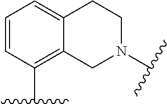 [SMILES: *c1cccc2c1CN(*)CC2]

which is optionally substituted at the 5-position with a group selected from hydroxyl, C₁₋₄ alkoxy, and cyano; or
Ar² is

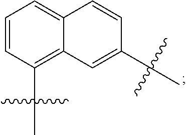 [SMILES: *c1ccc2cccc(*)c2c1]

or
Ar² is

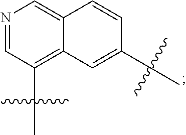 [SMILES: *c1ccc2cncc(*)c2c1]

or
Ar² is

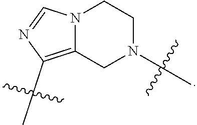 [SMILES: *c1ncn2c1CN(*)CC2]

In certain embodiments, Ar² of formula (IIa) or (IIb) is

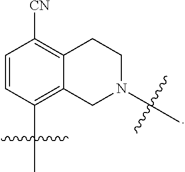 [SMILES: *c1ccc(C#N)c2c1CN(*)CC2]

In certain embodiments, Ar² of formula (IIa) or (IIb) is

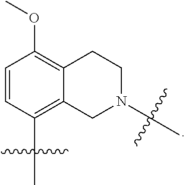 [SMILES: *c1ccc(OC)c2c1CN(*)CC2]

In certain embodiments, Ar² of formula (IIa) or (IIb) is

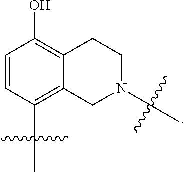 [SMILES: *c1ccc(O)c2c1CN(*)CC2]

In certain embodiments, Ar² of formula (IIa) or (IIb) is

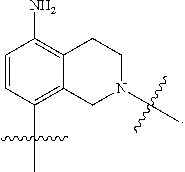 [SMILES: *c1ccc(N)c2c1CN(*)CC2]

In certain embodiments, Ar² of formula (IIa) or (IIb) is

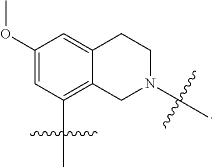 [SMILES: *c1cc(OC)cc2c1CN(*)CC2]

In certain embodiments, Ar² of formula (IIa) or (IIb) is

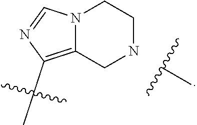

In certain embodiments, Ar² of formula (IIa) or (IIb) is

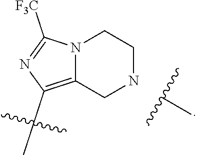

In certain embodiments, Ar² of formula (IIa) or (IIb) is

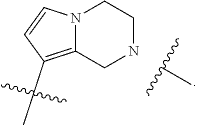

In certain embodiments, Ar² of formula (IIa) or (IIb) is

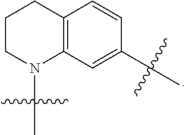 [SMILES: *c1ccc2c(c1)N(*)CCC2]

In certain embodiments, Ar² of formula (IIa) or (IIb) is

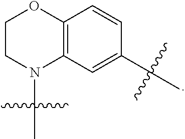 [SMILES: *c1ccc2c(c1)N(*)CCO2]

In certain embodiments, Ar² of formula (IIa) or (IIb) is

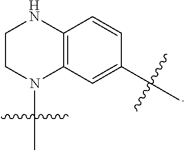 [SMILES: *c1ccc2c(c1)N(*)CCN2]

In certain embodiments, Ar² of formula (IIa) or (IIb) is

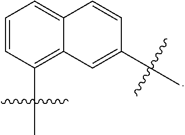 [SMILES: *c1ccc2cccc(*)c2c1]

In certain embodiments, Ar² of formula (IIa) or (IIb) is

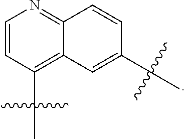 [SMILES: *c1ccc2nccc(*)c2c1]

In certain embodiments, Ar² of formula (IIa) or (IIb) is

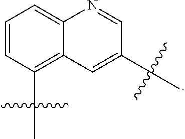 [SMILES: *c1cnc2cccc(*)c2c1]

In certain embodiments, Ar² of formula (IIa) or (IIb) is

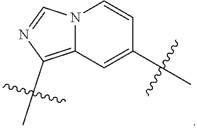 [SMILES: *c1ccn2cnc(*)c2c1]

In certain embodiments, Ar² of formula (IIa) or (IIb) is

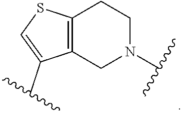 [SMILES: *c1csc2c1CN(*)CC2]

In certain embodiments, Ar² of formula (IIa) or (IIb) is

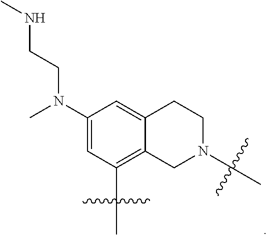 [SMILES: *c1cc(N(C)CCNC)cc2c1CN(*)CC2]

In certain embodiments, Ar² of formula (IIa) or (IIb) is

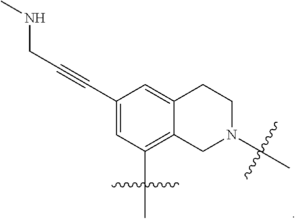 [SMILES: *c1cc(C#CCNC)cc2c1CN(*)CC2]

In certain embodiments, Ar² of formula (IIa) or (IIb) is

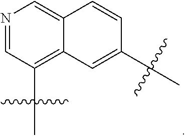 [SMILES: *c1ccc2cncc(*)c2c1]

In certain embodiments, Ar² of formula (IIa) or (IIb) is

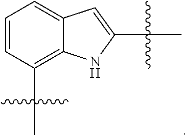 [SMILES: *c1cc2cccc(*)c2[nH]1]

In certain embodiments, Ar² of formula (IIa) or (IIb) is

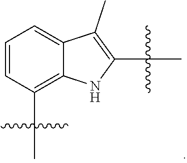 [SMILES: *c1[nH]c2c(*)cccc2c1C]

In certain embodiments, Ar² of formula (IIa) or (IIb) is

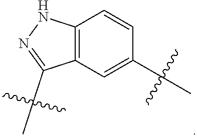 [SMILES: *c1ccc2[nH]nc(*)c2c1]

In certain embodiments, Ar² of formula (IIa) or (IIb) is

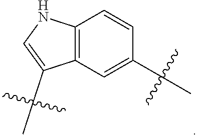 [SMILES: *c1ccc2[nH]cc(*)c2c1]

In certain embodiments, Ar² of formula (IIa) or (IIb) is

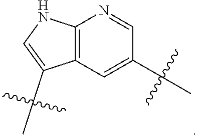 [SMILES: *c1cnc2[nH]cc(*)c2c1]

In certain embodiments, Ar² of formula (IIa) or (IIb) is

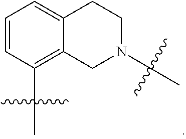 [SMILES: *c1cccc2c1CN(*)CC2]

In certain embodiments, Ar² of formula (IIa) is unsubstituted.

In certain embodiments, Ar² of formula (IIa) or (IIb) is

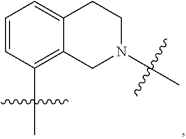 [SMILES: *c1cccc2c1CN(*)CC2]

which is substituted at the 5-position with a group selected from hydroxyl, C₁₋₄alkoxy, and cyano.

In certain embodiments, Z¹ of formula (IIa) or (IIb) is N.

In certain embodiments, R¹ of formula (IIa) or (IIb) is selected from methyl and chloro.

In certain embodiments, R² of formula (IIa) or (IIb) is selected from hydrogen and methyl. In particular embodiments, R² is hydrogen.

In certain embodiments, R⁴ of formula (IIa) or (IIb) is methyl.

In certain embodiments, R⁴ of formula (IIa) or (IIb) is (CF₂)₂OCH₃.

In certain embodiments, R⁴ of formula (IIa) or (IIb) is hydrogen.

In certain embodiments, R⁴ of formula (IIa) or (IIb) is monocyclic heterocyclyl, wherein the monocyclic heterocycloalkyl is substituted with one S(O)₂CH₃.

In certain embodiments, R⁴ of formula (IIa) or (IIb) is hydrogen or lower alkyl, wherein the lower alkyl is optionally substituted with C₁₋₄alkoxy or C(O)NR^{6a}R^{6b}.

In certain embodiments, R⁴ of formula (IIa) or (IIb) is lower alkyl, wherein the lower alkyl is substituted with C(O)NH₂.

In certain embodiments, R⁴ of formula (IIa) or (IIb) is lower alkyl, wherein the lower alkyl is substituted with S(O)₂NH₂.

In certain embodiments, R⁴ of formula (IIa) or (IIb) is lower alkyl, wherein the lower alkyl is substituted with hydroxy.

In certain embodiments, R⁴ of formula (IIa) or (IIb) is lower alkyl wherein the lower alkyl is substituted with C(O)N(CH₃)₂.

In certain embodiments, R⁴ of formula (IIa) or (IIb) is lower alkyl wherein the lower alkyl is substituted with C(O)NHCH₃.

In certain embodiments, R^{11a} and R^{11b} of formula (IIa) or (IIb) are the same. In a particular embodiment, R^{11a} and R^{11b} are each methyl. In another embodiment, R^{11a} and R^{11b} are each ethyl. In another embodiment, R^{11a} and R^{11b} are each methoxy.

In certain embodiments, R^{11a} and R^{11b} of formula (IIa) or (IIb) are independently selected from F, Br and Cl.

In certain embodiments, Z¹ is N, Z^{2a} is O, R¹ is methyl or chloro, R² is hydrogen, and Ar² is

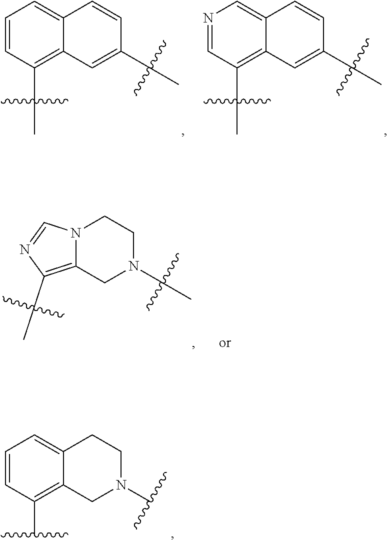 [SMILES: *c1ccc2cccc(*)c2c1 ; *c1ccc2cncc(*)c2c1 ; *c1ncn2c1CN(*)CC2 ; *c1cccc2c1CN(*)CC2]

wherein the

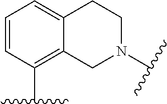 [SMILES: *c1cccc2c1CN(*)CC2]

is optionally substituted at the 5-position with a group selected from hydroxyl, C₁₋₄alkoxy, and cyano.

Certain embodiments pertain to a compound of formula (IIa). In certain embodiments, Z^{2a} of formula (IIa) is O.

In certain embodiments. Z^{2a} of formula (IIa) is CH₂ or O.

In certain embodiments, Z^{2a} of formula (IIa) is S.

In certain embodiments, Z^{2a} of formula (IIa) is CH₂.

In certain embodiments, Z^{2a} of formula (IIa) is NR⁶. In some such embodiments R⁶ is methyl.

In certain embodiments, Z^{2a} of formula (IIa) is NR⁶C(O). In some such embodiments R⁶ is hydrogen.

In certain embodiments, Z^{2a} of formula (IIa) is O, R¹³ is ethylene, and R⁴ is lower alkyl.

In certain embodiments. Z^{2a} of formula (IIa) is O, R¹³ is ethylene, and R⁴ is hydrogen or lower alkyl optionally substituted with C₁₋₄ alkoxy or C(O)NR^{6a}R^{6b}.

In certain embodiments, Z^{2a} of formula (IIa) is O, R¹³ is ethylene, and R⁴ is methyl.

In certain embodiments, Z^{2a} of formula (IIa) is O, R¹³ is ethylene, and R⁴ is hydrogen.

In certain embodiments, Z^{2a} of formula (IIa) is NR⁶C(O), R⁶ is hydrogen, R¹³ is methylene, and R⁴ is hydrogen.

In certain embodiments, Z^{2a} of formula (IIa) is S, R¹³ is ethylene, and R⁴ is hydrogen.

In certain embodiments, Z^{2a} of formula (IIa) is CH₂, R¹³ is ethylene, and R⁴ is hydrogen.

In certain embodiments, the group R¹³ in formula (IIa) is ethylene. In some such embodiments Z^{2a} is O.

In certain embodiments, the group R¹³ in formula (IIa) is propylene. In some such embodiments Z^{2a} is O.

In certain embodiments, the group R¹³ in formula (IIa) is selected from lower alkylene or lower heteroalkylene.

In certain embodiments, the group R¹³ in formula (IIa) is selected from (CH₂)₂O(CH₂)₂, (CH₂)₃O(CH₂)₂, (CH₂)₂O(CH₂)₃ and (CH₂)₃O(CH₂)₃. In some such embodiments Z^{2a} is O.

In certain embodiments, the group R¹³ in formula (IIa) is selected from (CH₂)₂(SO₂)(CH₂)₂, (CH₂)₃(SO₂)(CH₂)₂, (CH₂)₂(SO₂)(CH₂)₃ and (CH₂)₃(SO₂)(CH₂)₃. In some such embodiments Z^{2a} is O.

In certain embodiments, the group R¹³ in formula (IIa) is selected from (CH₂)₂(SO₂)(CH₂)₂, (CH₂)₃(SO₂)(CH₂)₃, (CH₂)₂(SO₂)(CH₂)₃ and (CH₂)₃(SO₂)(CH₂)₃. In some such embodiments Z^{2a} is O.

In certain embodiments, the group R¹³ in formula (IIa) is selected from (CH₂)₂S(CH₂)₂, (CH₂)₂S(CH₂)₃, (CH₂)₃S(CH₂)₂ and (CH₂)₃S(CH₂)₃. In some such embodiments Z^{2a} is O.

In certain embodiments, the group

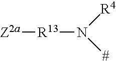

in formula (IIa) is

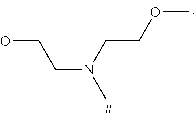

In certain embodiments, the group

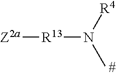

in formula (IIa) is

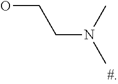

In certain embodiments, the group

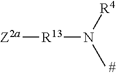

in formula (IIa) is

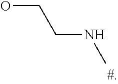

In certain embodiments, the group

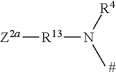

in formula (IIa) is

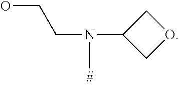

In certain embodiments, the group

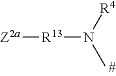

is selected from

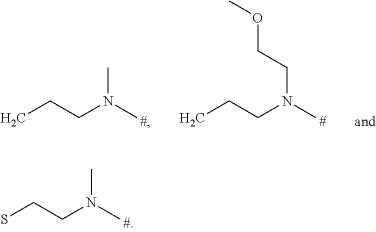

In certain embodiments, the group

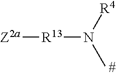

in formula (IIa) is

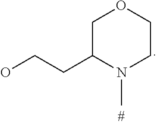

In certain embodiments, the group

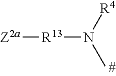

in formula (IIa) is selected from

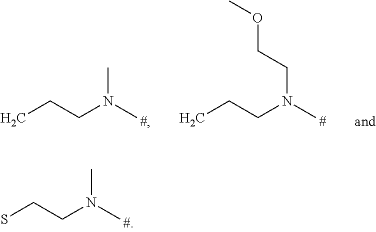

In certain embodiments, the group

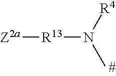

in formula (IIa) is

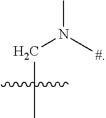 [SMILES: *CN(C)C]

In certain embodiments, the group

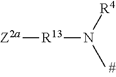

in formula (IIa) is

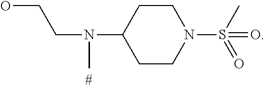

In certain embodiments, the group

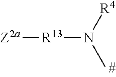

in formula (IIa) is

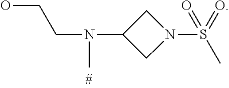

In certain embodiments, the group

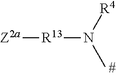

in formula (IIa) is

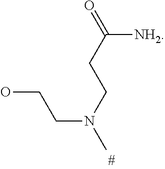

In certain embodiments, the group

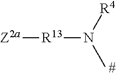

in formula (IIa) is

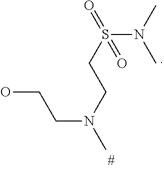

In certain embodiments, the group

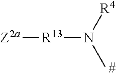

in formula (IIa) is

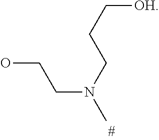

In certain embodiments, the group

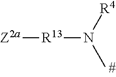

in formula (Ha) is

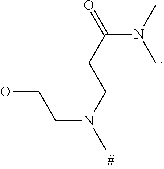

In certain embodiments, the group

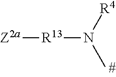

in formula (IIa) is

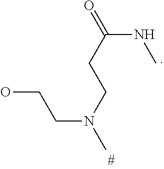

Certain embodiments pertain to a compound of formula (IIb).

In certain embodiments, the group Z^{a} in formula (IIb) is a bond. O, or NR⁶, or and R¹³ is ethylene or optionally substituted heterocyclyl.

In certain embodiments, the group Z^{2a} in formula (IIb) is NR⁶. In some such embodiments R⁶ is methyl.

In certain embodiments, the group Z^{2a} in formula (IIb) is NR⁶ and R¹³ is ethylene. In some such embodiments R⁶ is methyl.

In certain embodiments, the group Z^{2b} in formula (IIb) is O and R¹³ is ethylene. In some such embodiments R⁴ is methyl.

In certain embodiments, the group Z^{2b} in formula (IIb) is NR⁶, wherein the R⁶ group is taken together with an atom of R¹³ to form a ring having between 4 and 6 atoms. In some such embodiments the ring is a five membered ring.

In certain embodiments, the group Z^{2b} in formula (IIb) is methylene and the group R¹³ is methylene.

In certain embodiments, the group Z^{2b} in formula (IIb) is methylene and the group R¹³ is a bond.

In certain embodiments, the group Z^{2b} in formula (IIb) is oxygen and the group R¹³ is selected from (CH₂)₂O(CH₂)₂, (CH₂)₃O(CH₂)₂, (CH₂)₂O(CH₂)₃ and (CH₂)₃O(CH₂)₃. In some such embodiments R⁴ is methyl.

In certain embodiments, the group Z^{2c} in formula (IIb) is a bond and R¹² is OH.

In certain embodiments, the group Z^{2e} in formula (IIb) is a bond and R¹² is selected from F, Cl, Br and I.

In certain embodiments, the group Z^{2c} in formula (IIb) is a bond and R¹² is lower alkyl. In some such embodiments R¹² is methyl.

In certain embodiments, the group Z^{2a} in formula (IIb) is O and R¹² is a lower heteroalkyl. In some such embodiments R¹² is O(CH₂)₂OCH₃.

In certain embodiments, the group Z^{2c} in formula (IIb) is O and R¹² is lower alkyl optionally substituted with one or more halo or CM alkoxy.

In certain embodiments, the group Z^{2c} in formula (IIb) is O and R¹² is a lower alkyl. In particular embodiments R¹² is methyl.

In certain embodiments, the group Z^{2c} in formula (IIb) is S and R¹² is a lower alkyl. In some such embodiments R¹² is methyl.

Exemplary Bcl-xL inhibitors according to structural formulae (IIa)-(IIb) that may be used in the methods described herein in unconjugated form and/or included in the ADCs described herein include the following compounds, and/or a pharmaceutically acceptable salt thereof:

| Appln | Bcl-xL Inhibitory |
|---|---|
| Ex. No. | Compound |
| 1.1 | W3.01 |
| 1.2 | W3.02 |
| 1.3 | W3.03 |
| 1.4 | W3.04 |
| 1.5 | W3.05 |
| 1.6 | W3.06 |
| 1.7 | W3.07 |
| 1.8 | W3.08 |
| 1.9 | W3.09 |
| 1.10 | W3.10 |
| 1.11 | W3.11 |
| 1.12 | W3.12 |
| 1.13 | W3.13 |
| 1.14 | W3.14 |
| 1.15 | W3.15 |
| 1.16 | W3.16 |
| 1.17 | W3.17 |
| 1.18 | W3.18 |
| 1.19 | W3.19 |
| 1.20 | W3.20 |
| 1.21 | W3.21 |
| 1.22 | W3.22 |
| 1.23 | W3.23 |
| 1.24 | W3.24 |
| 1.25 | W3.25 |
| 1.26 | W3.26 |
| 1.27 | W3.27 |
| 1.28 | W3.28 |
| 1.29 | W3.29 |
| 1.30 | W3.30 |
| 1.31 | W3.31 |
| 1.32 | W3.32 |
| 1.33 | W3.33 |
| 1.34 | W3.34 |
| 1.35 | W3.35 |
| 1.36 | W3.36 |
| 1.37 | W3.37 |
| 1.38 | W3.38 |
| 1.39 | W3.39 |
| 1.40 | W3.40 |
| 1.41 | W3.41 |
| 1.42 | W3.42 |
| 1.43 | W3.43 |
| 1.44 (Control) | W3.44 |

Notably, when the Bcl-xL inhibitor of the present application is in conjugated form, the hydrogen corresponding to the # position of structural formula (IIa) or (IIb) is not present, forming a monoradical. For example, compound W3.01 (Example 1.1) is 6-[1-(1,3-benzothiazol-2-ylcarbamoyl)-1,2,3,4-tetrahydroquinolin-7-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid.

When it is in unconjugatcd form, it has the following structure:

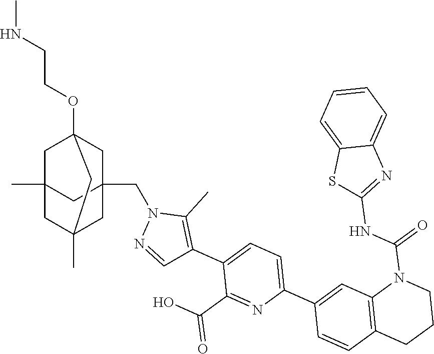 [SMILES: CNCCOC12CC3(C)CC(C)(CC(Cn4ncc(-c5ccc(-c6ccc7c(c6)N(C(=O)Nc6nc8ccccc8s6)CCC7)nc5C(=O)O)c4C)(C3)C1)C2]

When the same compound is included in the ADCs as shown in structural formula (IIa) or (IIb), the hydrogen corresponding to the # position is not present forming a monoradical.

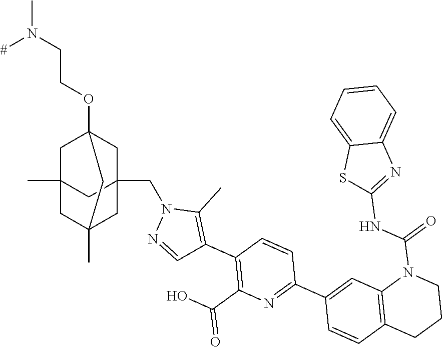

In certain embodiments, the Bcl-xL inhibitor is selected from the group consisting of W3.01, W3.02, W3.03, W3.04, W3.05, W3.06, W3.07, W3.08, W3.09, W3.10, W3.11, W3.12, W3.13. W3.14, W3.15, W3.16, W3.17, W3.18, W3.19, W3.20, W3.21, W3.22, W3.23, W3.24, W3.25, W3.26, W3.27, W3.28, W3.29, W3.30, W3.31, W3.32, W3.33, W3.34, W3.35, W3.36, W3.37, W3.38, W3.39, W3.40, W3.41, W3.42, W3.43, and pharmaceutically acceptable salts thereof (see Example 1 for compounds).

In certain embodiments, the ADC, or a pharmaceutically acceptable salt thereof, comprises a drug linked to an antibody by way of a linker, wherein the drug is a Bcl-xL inhibitor selected from the group consisting of W3.01, W3.02, W3.03, W3.04, W3.05, W3.06, W3.07, W3.08, W3.09, W3.10, W3.11, W3.12, W3.13, W3.14, W3.15, W3.16, W3.17, W3.18, W3.19, W3.20, W3.21, W3.22, W3.23, W3.24, W3.25, W3.26, W3.27, W3.28, W3.29, W3.30, W3.31, W3.32, W3.33, W3.34, W3.35, W3.36, W3.37, W3.38, W3.39, W3.40, W3.41, W3.42, W3.43.

In certain embodiments, the ADC, or a pharmaceutically acceptable salt thereof, the Bcl-xL inhibitor is selected from the group consisting of the following compounds modified in that the hydrogen corresponding to the # position of structural formula (IIa) or (IIb) is not present forming a monoradical:
6-[1-(1,3-benzothiazol-2-ylcarbamoyl)-1,2,3,4-tetrahydroquinolin-7-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
6-[4-(1,3-benzothiazol-2-ylcarbamoyl)-3,4-dihydro-2H-1,4-benzoxazin-6-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
6-[4-(1,3-benzothiazol-2-ylcarbamoyl)-1-methyl-1,2,3,4-tetrahydroquinoxalin-6-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
3-(1-{[3-(2-aminoethoxy)-5,7-dimethyltricyclo[3.3.1.1^{3,7}]dec-1-yl]methyl}-5-methyl-1H-pyrazol-4-yl)-6-[1-(1,3-benzothiazol-2-ylcarbamoyl)-5,6-dihydroimidazo[1,5-a]pyrazin-7(8H)-yl]pyridine-2-carboxylic acid;
3-(1-{[3-(2-aminoethoxy)-5,7-dimethyltricyclo[3.3.1.1^{3,7}]dec-1-yl]methyl}-5-methyl-1H-pyrazol-4-yl)-6-[1-(1,3-benzothiazol-2-ylcarbamoyl)-5-hydroxy-3,4-dihydroisoquinolin-2(1H)-yl]pyridine-2-carboxylic acid;
6-[8-(1,3-benzothiazol-2-ylcarbamoyl)naphthalen-2-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
3-[1-({3,5-dimethyl-7-[2-(methylamino)ethoxy]tricyclo[3.3.1.1^{3,7}]dec-1-yl}methyl)-5-methyl-1H-pyrazol-4-yl]-6-[8-([1,3]thiazolo[5,4-b]pyridin-2-ylcarbamoyl)naphthalen-2-yl]pyridine-2-carboxylic acid;
3-[1-({3,5-dimethyl-7-[2-(methylamino)ethoxy]tricyclo[3.3.1.1^{3,7}]dec-1-yl}methyl)-5-methyl-1H-pyrazol-4-yl]-6-[8-([1,3]thiazolo[4,5-b]pyridin-2-ylcarbamoyl)naphthalen-2-yl]pyridine-2-carboxylic acid;
6-[8-(1,3-benzothiazol-2-ylcarbamoyl)-5-methoxy-3,4-dihydroisoquinolin-2(1H)-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
6-[5-(1,3-benzothiazol-2-ylcarbamoyl)quinolin-3-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
6-[4-(1,3-benzothiazol-2-ylcarbamoyl)quinolin-6-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
6-[8-(1,3-benzothiazol-2-ylcarbamoyl)-5-methoxy-3,4-dihydroisoquinolin-2(1H)-yl]-3-{1-[(3-{2-[(2-methoxyethyl)amino]ethoxy}-5,7-dimethyltricyclo[3.3.1.1^{3,7}]dec-1-yl)methyl]-5-methyl-1H-pyrazol-4-yl}pyridine-2-carboxylic acid;
3-(1-{[3-(2-aminoethoxy)-5,7-dimethyltricyclo[3.3.1.1^{3,7}]dec-1-yl]methyl}-5-methyl-1H-pyrazol-4-yl)-6-[8-(1,3-benzothiazol-2-ylcarbamoyl)-5-cyano-3,4-dihydroisoquinolin-2(1H)-yl]pyridine-2-carboxylic acid;
6-[1-(1,3-benzothiazol-2-ylcarbamoyl)-1,2,3,4-tetrahydroquinolin-7-yl]-3-{1-[(3-{2-[(2-methoxyethyl)amino]ethoxy}-5,7-dimethyltricyclo[3.3.1.1^{3,7}]dec-1-yl)methyl]-5-methyl-1H-pyrazol-4-yl}pyridine-2-carboxylic acid;
6-[8-(1,3-benzothiazol-2-ylcarbamoyl)naphthalen-2-yl]-3-{1-[(3-{2-[(2-methoxyethyl)amino]ethoxy}-5,7-dimethyltricyclo[3.3.1.1^{3,7}]dec-1-yl)methyl]-5-methyl-1H-pyrazol-4-yl}pyridine-2-carboxylic acid;
6-[8-(1,3-benzothiazol-2-ylcarbamoyl)-3,4-dihydroisoquinolin-2(1H)-yl]-3-[1-({3,5-dimethyl-7-[2-(oxetan-3-ylamino)ethoxy]tricyclo[3.3.1.1^{3,7}]dec-1-yl}methyl)-5-methyl-1H-pyrazol-4-yl]pyridine-2-carboxylic acid;
6-[6-(3-aminopyrrolidin-1-yl)-8-(1,3-benzothiazol-2-ylcarbamoyl)-3,4-dihydroisoquinolin-2(1H)-yl]-3-(1-{[3-(2-methoxyethoxy)-5,7-dimethyltricyclo[3.3.1.1^{3,7}]dec-1-yl]methyl}-5-methyl-1H-pyrazol-4-yl)pyridine-2-carboxylic acid;
6-[8-(1,3-benzothiazol-2-ylcarbamoyl)-3,4-dihydroisoquinolin-2(1H)-yl]-3-{1-[(3,5-dimethyl-7-{2-[(2-sulfamoylethyl)amino]ethoxy}tricyclo[3.3.1.1^{3,7}]dec-1-yl)methyl]-5-methyl-1H-pyrazol-4-yl}pyridine-2-carboxylic acid;
3-(1-{[3-(2-aminoethoxy)-5,7-dimethyltricyclo[3.3.1.1^{3,7}]dec-1-yl]methyl}-5-methyl-1H-pyrazol-4-yl)-6-[3-(1,3-benzothiazol-2-ylcarbamoyl)-6,7-dihydrothieno[3,2-c]pyridin-5(4H)-yl]pyridine-2-carboxylic acid;
3-(1-{[3-(2-aminoethoxy)-5,7-dimethyltricyclo[3.3.1.1^{3,7}]dec-1-yl]methyl}-5-methyl-1H-pyrazol-4-yl)-6-[1-(1,3-benzothiazol-2-ylcarbamoyl)-3-(trifluoromethyl)-5,6-dihydroimidazo[1,5-a]pyrazin-7(8H)-yl]pyridine-2-carboxylic acid;
6-[8-(1,3-benzothiazol-2-ylcarbamoyl)-6-{methyl[2-(methylamino)ethyl]amino}-3,4-dihydroisoquinolin-2(1H)-yl]-3-(1-{[3-(2-methoxyethoxy)-5,7-dimethyltricyclo[3.3.1.1^{3,7}]dec-1-yl]methyl}-5-methyl-1H-pyrazol-4-yl)pyridine-2-carboxylic acid;
6-[8-(1,3-benzothiazol-2-ylcarbamoyl)-6-methoxy-3,4-dihydroisoquinolin-2(1H)-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
3-(1-{[3-(2-aminoethoxy)-5,7-dimethyltricyclo[3.3.1.1^{3,7}]dec-1-yl]methyl}-5-methyl-1H-pyrazol-4-yl)-6-[4-(1,3-benzothiazol-2-ylcarbamoyl)quinolin-6-yl]pyridine-2-carboxylic acid;
6-[5-amino-8-(1,3-benzothiazol-2-ylcarbamoyl)-3,4-dihydroisoquinolin-2(1H)-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
6-[8-(1,3-benzothiazol-2-ylcarbamoyl)-6-[3-(methylamino)prop-1-yn-1-yl]-3,4-dihydroisoquinolin-2(1H)-yl]-3-(1-{[3-(2-methoxyethoxy)-5,7-dimethyltricyclo[3.3.1.1^{3,7}]dec-1-yl]methyl}-5-methyl-1H-pyrazol-4-yl)pyridine-2-carboxylic acid;
6-[4-(1,3-benzothiazol-2-ylcarbamoyl)isoquinolin-6-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
6-[7-(1,3-benzothiazol-2-ylcarbamoyl)-1H-indol-2-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
3-(1-{[3-(2-aminoethoxy)-5,7-dimethyltricyclo[3.3.1.1^{3,7}]dec-1-yl]methyl}-5-methyl-1H-pyrazol-4-yl)-6-[7-(1,3-benzothiazol-2-ylcarbamoyl)-1H-indol-2-yl]pyridine-2-carboxylic acid;
6-[7-(1,3-benzothiazol-2-ylcarbamoyl)-3-methyl-1H-indol-2-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
6-[8-(1,3-benzothiazol-2-ylcarbamoyl)-3,4-dihydroisoquinolin-2(1H)-yl]-3-(1-{[3,5-dimethyl-7-(2-{[l-(methylsulfonyl)piperidin-4-yl]amino}ethoxy)tricyclo[3.3.1.1^{3,7}]dec-1-yl]methyl}-5-methyl-1H-pyrazol-4-yl)pyridine-2-carboxylic acid;
6-[8-(1,3-benzothiazol-2-ylcarbamoyl)-3,4-dihydroisoquinolin-2(1H)-yl]-3-(1-{[3,5-dimethyl-7-(2-{[l-(methylsulfonyl)azetidin-3-yl]amino}ethoxy)tricyclo[3.3.1.1^{3,7}]dec-1-yl]methyl}-5-methyl-1H-pyrazol-4-yl)pyridine-2-carboxylic acid;
3-{1-[(3-{2-[(3-amino-3-oxopropyl)amino]ethoxy}-5,7-dimethyltricyclo[3.3.1.1^{3,7}]dec-1-yl)methyl]-5-methyl-1H-pyrazol-4-yl}-6-[8-(1,3-benzothiazol-2-ylcarbamoyl)-3,4-dihydroisoquinolin-2(1H)-yl]pyridine-2-carboxylic acid;
6-[3-(1,3-benzothiazol-2-ylcarbamoyl)-1H-indazol-5-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
6-[3-(1,3-benzothiazol-2-ylcarbamoyl)-1H-indol-5-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
6-[3-(1,3-benzothiazol-2-ylcarbamoyl)-1H-pyrrolo[2,3-b]pyridin-5-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
6-(8-(benzo[d]thiazol-2-ylcarbamoyl)-3,4-dihydroisoquinolin-2(1H)-yl)-3-(1-((3-(2-((2-(N,N-dimethylsulfamoyl)ethyl)amino)ethoxy)-5,7-dimethyladamantan-1-yl)methyl)-5-methyl-1H-pyrazol-4-yl)picolinic acid:
6-[8-(1,3-benzothiazol-2-ylcarbamoyl)naphthalen-2-yl]-3-{1-[(3-{2-[(3-hydroxypropyl)amino]ethoxy}-5,7-dimethyltricyclo[3.3.1.1^{3,7}]dec-1-yl)methyl]-5-methyl-1H-pyrazol-4-yl}pyridine-2-carboxylic acid;
6-[8-(1,3-benzothiazol-2-ylcarbamoyl)-3,4-dihydroisoquinolin-2(1H)-yl]-3-(1-{[3-(2-{[3-(dimethylamino)-3-oxopropyl]amino}ethoxy)-5,7-dimethyltricyclo[3.3.1.1^{3,7}]dec-1-yl]methyl}-5-methyl-1H-pyrazol-4-yl)pyridine-2-carboxylic acid;
6-[8-(1,3-benzothiazol-2-ylcarbamoyl)-3,4-dihydroisoquinolin-2(1H)-yl]-3-(1-{[3,5-dimethyl-7-(2-{[3-(methylamino)-3-oxopropyl]amino}ethoxy)tricyclo[3.3.1.1^{3,7}]dec-1-yl]methyl}-5-methyl-1H-pyrazol-4-yl)pyridine-2-carboxylic acid;
3-(1-{[3-(2-aminoacetamido)-5,7-dimethyltricyclo[3.3.1.1^{3,7}]decan-1-yl]methyl}-5-methyl-1H-pyrazol-4-yl)-6-{8-[(1,3-benzothiazol-2-yl)carbamoyl]-3,4-dihydroisoquinolin-2(1H)-yl}pyridine-2-carboxylic acid;
3-[1-({3-[(2-aminoethyl)sulfanyl]-5,7-dimethyltricyclo[3.3.1.1^{3,7}]dec-1-yl}methyl)-5-methyl-1H-pyrazol-4-yl]-6-[8-(1,3-benzothiazol-2-ylcarbamoyl)-3,4-dihydroisoquinolin-2(1H)-yl]pyridine-2-carboxylic acid;
3-(1-{[3-(3-aminopropyl)-5,7-dimethyltricyclo[3.3.1.1^{3,7}]dec-1-yl]methyl}-5-methyl-1H-pyrazol-4-yl)-6-[8-(1,3-benzothiazol-2-ylcarbamoyl)-3,4-dihydroisoquinolin-2(1H)-yl]pyridine-2-carboxylic acid; and
3-(1-{[3-(2-aminoethoxy)-5,7-dimethyltricyclo[3.3.1.1^{3,7}]decan-1-yl]methyl}-5-methyl-1H-pyrazol-4-yl)-6-{5-[(1,3-benzothiazol-2-yl)carbamoyl]quinolin-3-yl}pyridine-2-carboxylic acid.

The Bcl-xL inhibitors bind to and inhibit anti-apoptotic Bcl-xL proteins, inducing apoptosis. The ability of specific Bcl-xL inhibitors according to structural formulae (IIa)-(IIb) to bind to and inhibit Bcl-xL activity may be confirmed in standard binding and activity assays, including, for example, the TR-FRET Bcl-xL binding assays described in Tao et al., 2014, ACS Med. Chem. Lett., 5:1088-1093.

Bcl-xL inhibitory activity may also be confirmed in standard cell-based cytotoxicity assays, such as the FL5.12 cellular and Molt-4 cytotoxicity assays described in Tao et al., 2014. ACS Med. Chem. Lett., 5:1088-1093. A specific Molt-4 cellular cytotoxicity assay that may be used to confirm Bcl-xL inhibitory activity of specific Bcl-xL inhibitors that are able to permeate cell membranes is provided in Example 5, below. Typically, such cell-permeable Bcl-xL inhibitors will exhibit an EC₅₀ of less than about 500 nM in the Molt-4 cytotoxicity assay of Example 5, but may exhibit a significantly lower EC₅₀, for example an EC₅₀ of less than about 250, 100, 50, 20, 10 or even 5 nM.

The process of mitochondrial outer-membrane permeabilization (MOMP) is controlled by the Bcl-2 family proteins. Specifically, MOMP is promoted by the pro-apoptotic Bcl-2 family proteins Bax and Bak which, upon activation oligomerize on the outer mitochondrial membrane and form pores, leading to release of cytochrome c (cyt c). The release of cyt c triggers formulation of the apoptosome which, in turn, results in caspase activation and other events that commit the cell to undergo programmed cell death (see, Goldstein et al., 2005, Cell Death and Differentiation 12:453-462). The oligomerization action of Bax and Bak is antagonized by the anti-apoptotic Bcl-2 family members, including Bcl-2 and Bcl-xL. Bcl-xL inhibitors, in cells that depend upon Bcl-xL for survival, can cause activation of Bax and/or Bak, MOMP, release of cyt c and downstream events leading to apoptosis. The process of cyt c release can be assessed via western blot of both mitochondrial and cytosolic fractions of cytochrome c in cells and used as a proxy measurement of apoptosis in cells.

As a means of detecting Bcl-xL inhibitory activity and consequent release of cyt c, the cells can be treated with an agent that causes selective pore formation in the plasma, but not mitochondrial, membrane. Specifically, the cholesterol/phospholipid ratio is much higher in the plasma membrane than the mitochondrial membrane. As a result, short incubation with low concentrations of the cholesterol-directed detergent digitonin selectively permeabilizes the plasma membrane without significantly affecting the mitochondrial membrane. This agent forms insoluble complexes with cholesterol leading to the segregation of cholesterol from its normal phospholipid binding sites. This action, in turn, leads to the formation of holes about 40-50 Å wide in the lipid bilaver. Once the plasma membrane is permeabilized, cytosolic components able to pass over digitonin-formed holes can be washed out, including the cytochrome C that was released from mitochondria to cytosol in the apoptotic cells (Campos, 2006. Cytometry A 69(6):515-523).

Although many of the Bcl-xL inhibitors of structural formulae (IIa)-(IIb) selectively or specifically inhibit Bcl-xL over other anti-apoptotic Bcl-2 family proteins, selective and/or specific inhibition of Bcl-xL is not necessary. The Bcl-xL inhibitors and ADCs comprising the compounds may also, in addition to inhibiting Bcl-xL, inhibit one or more other anti-apoptotic Bcl-2 family proteins, such as, for example, Bcl-2. In some embodiments, the Bcl-xL inhibitors and/or ADCs are selective and/or specific for Bcl-xL. By specific or selective is meant that the particular Bcl-xL inhibitor and/or ADC binds or inhibits Bcl-xL to a greater extent than Bcl-2 under equivalent assay-conditions. In specific embodiments, the Bcl-xL inhibitors and/or ADCs exhibit in the range of about 10-fold, 100-fold, or even greater specificity or selectivity for Bcl-xL than Bcl-2 in binding assays.

### III.A.2. Bcl-xL Linkers

In the ADCs described herein, the Bcl-xL inhibitors (described in Section III. A) are linked to the anti-CD98 antibody by way of linkers. The linker linking a Bcl-xL inhibitor to the anti-CD98 antibody of an ADC may be short long, hydrophobic, hydrophilic, flexible or rigid, or may be composed of segments that each independently has one or more of the above-mentioned properties such that the linker may include segments having different properties. The linkers may be polyvalent such that they covalently link more than one Bcl-xL inhibitor to a single site on the antibody, or monovalent such that covalently they link a single Bcl-xL inhibitor to a single site on the antibody.

As will be appreciated by skilled artisans, the linkers link the Bcl-xL inhibitors to the anti-CD98 antibody by forming a covalent linkage to the Bcl-xL inhibitor at one location and a covalent linkage to antibody at another. The covalent linkages are formed by reaction between functional groups on the linker and functional groups on the inhibitors and antibody. As used herein, the expression “linker” is intended to include (i) unconjugatcd forms of the linker that include a functional group capable of covalently linking the linker to a Bcl-xL inhibitor and a functional group capable of covalently linking the linker to an anti-CD98 antibody; (ii) partially conjugated forms of the linker that include a functional group capable of covalently linking the linker to an anti-CD98 antibody and that is covalently linked to a Bcl-xL inhibitor, or vice versa; and (iii) fully conjugated forms of the linker that is covalently linked to both a Bcl-xL inhibitor and an anti-CD98 antibody. In some specific embodiments of intermediate synthons and ADCs described herein, moieties comprising the functional groups on the linker and covalent linkages formed between the linker and antibody are specifically illustrated as R^{x} and LK, respectively. One embodiment pertains to an ADC formed by contacting an antibody that binds a cell surface receptor or tumor associated antigen expressed on a tumor cell with a synthon described herein under conditions in which the synthon covalently links to the anti-CD98 antibody. One embodiment pertains to a method of making an ADC formed by contacting a synthon described herein under conditions in which the synthon covalently links to the anti-CD98 antibody. One embodiment pertains to a method of inhibiting Bcl-xL activity in a cell that expresses Bcl-xL, comprising contacting the cell with an ADC described herein that is capable of binding the cell, under conditions in which the ADC binds the cell.

Exemplary polyvalent linkers that may be used to link many Bcl-xL inhibitors to an antibody are described, for example, in U.S. Pat. No. 8,399,512; U.S. Published Application No. 2010/0152725; U.S. Pat. Nos. 8,524,214; 8,349,308; U.S. Published Application No. 2013/189218; U.S. Published Application No. 2014/017265; WO 2014/093379; WO 2014/093394; WO 2014/093640, the contents of which are incorporated herein by reference in their entireties. For example, the Fleximer® linker technology developed by Mersana et al, has the potential to enable high-DAR ADCs with good physicochemical properties. As shown below, the Fleximer® linker technology is based on incorporating drug molecules into a solubilizing poly-acetal backbone via a sequence of ester bonds. The methodology renders highly-loaded ADCs (DAR up to 20) whilst maintaining good physicochemical properties. This methodology could be utilized with Bcl-xL inhibitors as shown in the Scheme below.

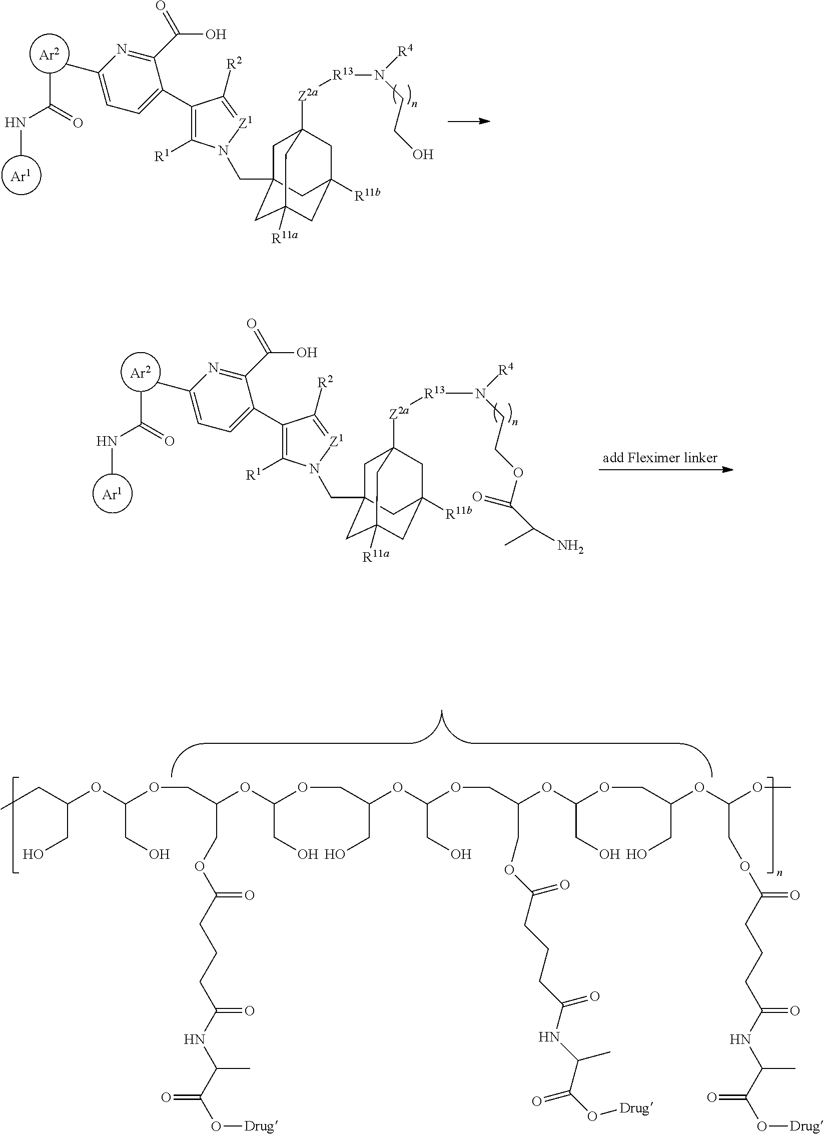

To utilize the Fleximer® linker technology depicted in the scheme above, an aliphatic alcohol can be present or introduced into the Bcl-xL inhibitor. The alcohol moiety is then conjugated to an alanine moiety, which is then synthetically incorporated into the Fleximer® linker. Liposomal processing of the ADC in vitro releases the parent alcohol-containing drug.

Additional examples of dendritic type linkers can be found in US 2006/116422; US 2005/271615; de Groot et al., (2003) Angew. Chem. Int. Ed. 42:4490-4494; Amir et al., (2003) Angew. Chem. Int. Ed. 42:4494-4499; Shamis et al., (2004) J. Am. Chem. Soc. 126:1726-1731; Sun et al., (2002) Bioorganic & Medicinal Chemistry Letters 12:2213-2215; Sun et al., (2003) Bioorganic & Medicinal Chemistry 11:1761-1768; King et al., (2002) Tetrahedron Letters 43:1987-1990.

Exemplary monovalent linkers that may be used are described, for example, in Nolting, 2013, Antibody-Drug Conjugates, Methods in Molecular Biology 1045:71-100; Kitson et al., 2013. CROs/CMOs—Chemica Oggi—Chemistry Today 31(4): 30-36; Ducry et al., 2010, Bioconjugate Chem. 21:5-13; Zhao et al., 2011, J. Med. Chem. 54:3606-3623; U.S. Pat. Nos. 7,223,837; 8,568,728; 8,535,678; and WO2004010957, the content of each of which is incorporated herein by reference in their entireties.

By way of example and not limitation, some cleavable and noncleavable linkers that may be included in the ADCs described herein are described below.

### Cleavable Linkers

In certain embodiments, the linker selected is cleavable in vitro and in vivo. Cleavable linkers may include chemically or enzymatically unstable or degradable linkages. Cleavable linkers generally rely on processes inside the cell to liberate the drug, such as reduction in the cytoplasm, exposure to acidic conditions in the lysosome, or cleavage by specific proteases or other enzymes within the cell. Cleavable linkers generally incorporate one or more chemical bonds that are either chemically or enzymatically cleavable while the remainder of the linker is noncleavable.

In certain embodiments, a linker comprises a chemically labile group such as hydrazone and/or disulfide groups. Linkers comprising chemically labile groups exploit differential properties between the plasma and some cytoplasmic compartments. The intracellular conditions to facilitate drug release for hydrazone containing linkers are the acidic environment of endosomes and lysosomes, while the disulfide containing linkers are reduced in the cytosol, which contains high thiol concentrations, e.g., glutathione. In certain embodiments, the plasma stability of a linker comprising a chemically labile group may be increased by introducing steric hindrance using substituents near the chemically labile group.

Acid-labile groups, such as hydrazone, remain intact during systemic circulation in the blood's neutral pH environment (pH 7.3-7.S) and undergo hydrolysis and release the drug once the ADC is internalized into mildly acidic endosomal (pH 5.0-6.5) and lysosomal (pH 4.5-5.0) compartments of the cell. This pH dependent release mechanism has been associated with nonspecific release of the drug. To increase the stability of the hydrazone group of the linker, the linker may be varied by chemical modification, e.g., substitution, allowing tuning to achieve more efficient release in the lysosome with a minimized loss in circulation.

Hydrazone-containing linkers may contain additional cleavage sites, such as additional acid-labile cleavage sites and/or enzymatically labile cleavage sites. ADCs including exemplary hydrazone-containing linkers include the following structures:

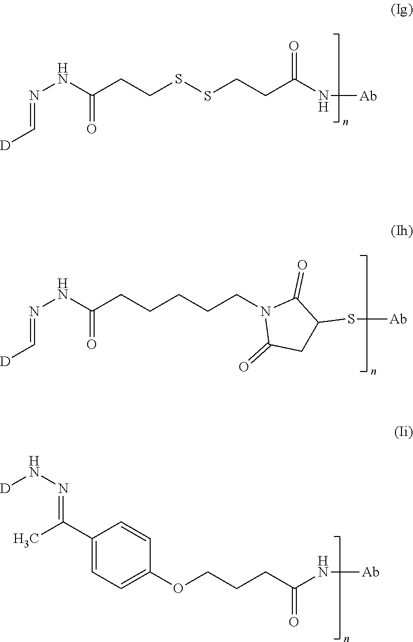

wherein D and Ab represent the drug and Ab, respectively, and n represents the number of drug-linkers linked to the anti-CD98 antibody. In certain linkers such as linker (Ig), the linker comprises two cleavable groups—a disulfide and a hydrazone moiety. For such linkers, effective release of the unmodified free drug requires acidic pH or disulfide reduction and acidic pH. Linkers such as (Ih) and (Ii) have been shown to be effective with a single hydrazone cleavage site.

Other acid-labile groups that may be included in linkers include cis-aconityl-containing linkers, cis-Aconityl chemistry uses a carboxylic acid juxtaposed to an amide bond to accelerate amide hydrolysis under acidic conditions.

Cleavable linkers may also include a disulfide group. Disulfides are thermodynamically stable at physiological pH and are designed to release the drug upon internalization inside cells, wherein the cytosol provides a significantly more reducing environment compared to the extracellular environment. Scission of disulfide bonds generally requires the presence of a cytoplasmic thiol cofactor, such as (reduced) glutathione (GSH), such that disulfide-containing linkers are reasonable stable in circulation, selectively releasing the drug in the cytosol. The intracellular enzyme protein disulfide isomerase, or similar enzymes capable of cleaving disulfide bonds, may also contribute to the preferential cleavage of disulfide bonds inside cells. GSH is reported to be present in cells in the concentration range of 0.5-10 mM compared with a significantly lower concentration of GSH or cysteine, the most abundant low-molecular weight thiol, in circulation at approximately 5 μM. Tumor cells, where irregular blood flow leads to a hypoxic state, result in enhanced activity of reductive enzymes and therefore even higher glutathione concentrations. In certain embodiments, the in vivo stability of a disulfide-containing linker may be enhanced by chemical modification of the linker, e.g., use of steric hindrance adjacent to the disulfide bond.

ADCs including exemplary disulfide-containing linkers include the following structures:

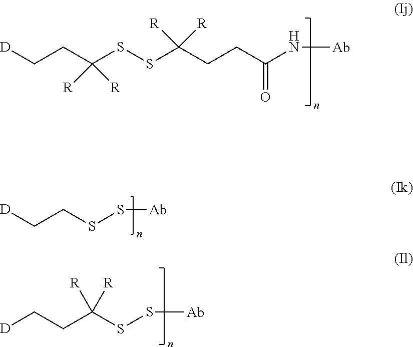

wherein D and Ab represent the drug and antibody, respectively, n represents the number of drug-linkers linked to the anti-CD98 antibody and R is independently selected at each occurrence from hydrogen or alkyl, for example. In certain embodiments, increasing steric hindrance adjacent to the disulfide bond increases the stability of the linker. Structures such as (Ij) and (II) show increased in vivo stability when one or more R groups is selected from a lower alkyl such as methyl.

Another type of linker that may be used is a linker that is specifically cleaved by an enzyme. In one embodiment, the linker is cleavable by a lysosomal enzyme. Such linkers are typically peptide-based or include peptidic regions that act as substrates for enzymes. Peptide based linkers tend to be more stable in plasma and extracellular milieu than chemically labile linkers. Peptide bonds generally have good serum stability, as lysosomal proteolytic enzymes have very low activity in blood due to endogenous inhibitors and the unfavorably high pH value of blood compared to lysosomes. Release of a drug from an anti-CD98 antibody occurs specifically due to the action of lysosomal proteases, e.g., cathepsin and plasmin. These proteases may be present at elevated levels in certain tumor tissues. In certain embodiments, the linker is cleavable by a lysosomal enzyme. In certain embodiments, the linker is cleavable by a lysosomal enzyme, and the lysosomal enzyme is Cathepsin B. In certain embodiments, the linker is cleavable by a lysosomal enzyme, and the lysosomal enzyme is β-glucuronidase or β-galactosidase. In certain embodiments, the linker is cleavable by a lysosomal enzyme, and the lysosomal enzyme is β-glucuronidase. In certain embodiments, the linker is cleavable by a lysosomal enzyme, and the lysosomal enzyme is β-galactosidase.

In exemplary embodiments, the cleavable peptide is selected from tetrapeptides such as Gly-Phe-Leu-Gly (SEQ ID NO: 166). Ala-Leu-Ala-Leu (SEQ ID NO: 167) or dipeptides such as Val-Cit, Val-Ala, and Phe-Lys. In certain embodiments, dipeptides are preferred over longer polypeptides due to hydrophobicity of the longer peptides.

A variety of dipeptide-based cleavable linkers useful for linking drugs such as doxorubicin, mitomycin, camptothecin, tallysomycin and auristatin/auristatin family members to antibodies have been described (see, Dubowchik et al., 1998, J. Org. Chem. 67:1866-1872; Dubowchik et al., 1998. Bioorg. Med. Chem. Lett. 8:3341-3346; Walker et al., 2002, Bioorg. Med Chem. Lett. 12:217-219; Walker et al., 2004. Bioorg. Med. Chem. Lett. 14:4323-4327; and Francisco et al., 2003, Blood 102:1458-1465, the contents of each of which are incorporated herein by reference). All of these dipeptide linkers, or modified versions of these dipeptide linkers, may be used in the ADCs described herein. Other dipeptide linkers that may be used include those found in ADCs such as Seattle Genetics' Brentuximab Vendotin SGN-35 (Adcetris™). Seattle Genetics SGN-75 (anti-CD-70, MC-monomethyl auristatin F(MMAF). Celldex Therapeutics glembatumumab (CDX-011) (anti-NMB, Val-Cit-monomethyl auristatin E(MMAE), and Cytogen PSMA-ADC (PSMA-ADC-1301) (anti-PSMA, Val-Cit-MMAE).

Enzymatically cleavable linkers may include a self-immolative spacer to spatially separate the drug from the site of enzymatic cleavage. The direct attachment of a drug to a peptide linker can result in proteolytic release of an amino acid adduct of the drag, thereby impairing its activity. The use of a self-immolative spacer allows for the elimination of the fully active, chemically unmodified drug upon amide bond hydrolysis.

One self-immolative spacer is the bifunctional para-aminobenzyl alcohol group, which is linked to the peptide through the amino group, forming an amide bond, while amine containing drugs may be attached through carbamate functionalities to the benzylic hydroxyl group of the linker (to give a p-amidobenzylcarbamate, PABC) The resulting prodrugs are activated upon protease-mediated cleavage, leading to a 1,6-elimination reaction releasing the unmodified drug, carbon dioxide, and remnants of the linker group. The following scheme depicts the fragmentation of p-amidobenzyl carbamate and release of the drug:

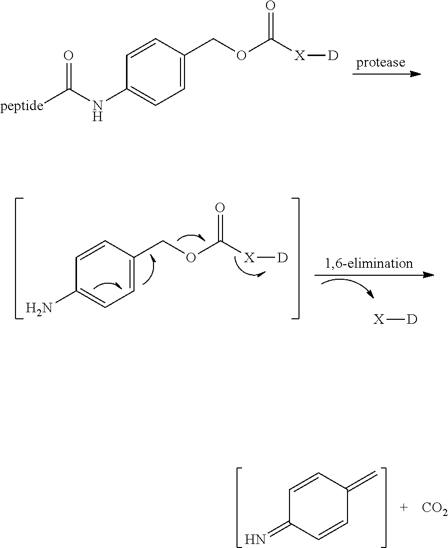

wherein X-D represents the unmodified drug.

Heterocyclic variants of this self-immolative group have also been described. See U.S. Pat. No. 7,989,434.

In certain embodiments, the enzymatically cleavable linker is a β-glucuronic acid-based linker. Facile release of the drug may be realized through cleavage of the β-glucuronide glycosidic bond by the lysosomal enzyme β-glucuronidase. This enzyme is present abundantly within lysosomes and is overexpressed in some tumor types, while the enzyme activity outside cells is low. β-Glucuronic acid-based linkers may be used to circumvent the tendency of an ADC to undergo aggregation due to the hydrophilic nature of β-glucuronides. In certain embodiments, β-glucuronic acid-based linkers are preferred as linkers for ADCs linked to hydrophobic drugs. The following scheme depicts the release of the drug from and ADC containing a β-glucuronic acid-based linker:

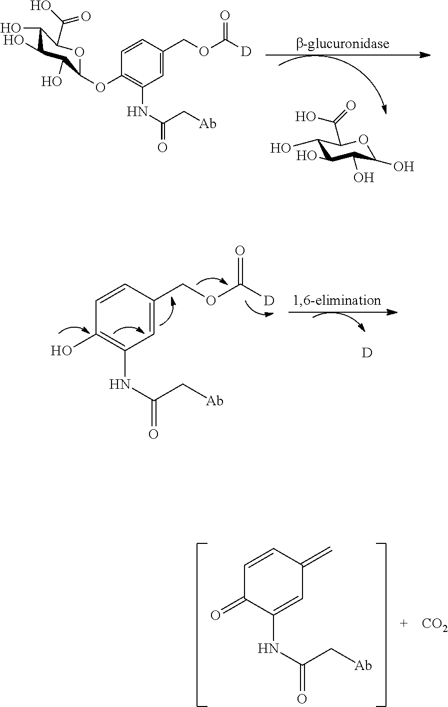

A variety of cleavable β-glucuronic acid-based linkers useful for linking drags such as auristatins, camptothecin and doxorubicin analogues. CBI minor-groove binders, and psymberin to antibodies have been described (see, Jeffrey et al., 2006, Bioconjug. Chem. 17:831-840; Jeffrey et al., 2007, Bioorg. Med Chem. Lett. 17:2278-2280; and Jiang et al., 2005, J. Am. Chem. Soc. 127:11254-11255, the contents of each of which are incorporated herein by reference). All of these β-glucuronic acid-based linkers may be used in the ADCs described herein. In certain embodiments, the enzymatically cleavable linker is a β-galactoside-based linker. β-Galactoside is present abundantly within lysosomes, while the enzyme activity outside cells is low.

Additionally, Bcl-xL inhibitors containing a phenol group can be covalently bonded to a linker through the phenolic oxygen. One such linker, described in U.S. Patent App. No. 2009/0318668, relies on a methodology in which a diamino-ethane “SpaceLink” is used in conjunction with traditional “PABO”-based self-immolative groups to deliver phenols. The cleavage of the linker is depicted schematically below using a Bcl-xL inhibitor of the disclosure.

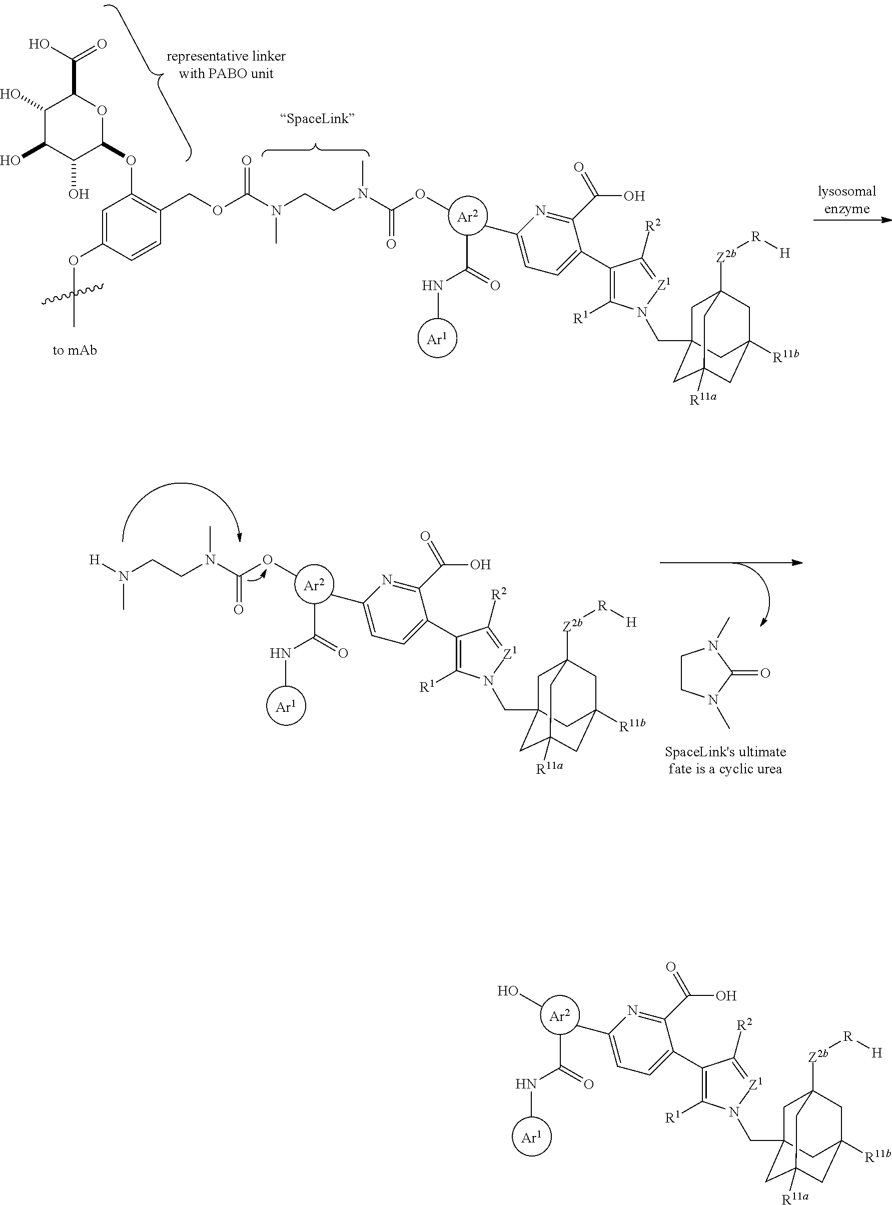

Cleavable linkers may include noncleavable portions or segments, and/or cleavable segments or portions may be included in an otherwise non-cleavable linker to render it cleavable. By way of example only, polyethylene glycol (PEG) and related polymers may include cleavable groups in the polymer backbone. For example, a polyethylene glycol or polymer linker may include one or more cleavable groups such as a disulfide, a hydrazone or a dipeptide.

Other degradable linkages that may be included in linkers include ester linkages formed by the reaction of PEG carboxylic acids or activated PEG carboxylic acids with alcohol groups on a biologically active agent wherein such ester groups generally hydrolyze under physiological conditions to release the biologically active agent. Hydrolytically degradable linkages include, but are not limited to, carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including but not limited to, at the end of a polymer, and a 5′ hydroxyl group of an oligonucleotide.

In certain embodiments, the linker comprises an enzymatically cleavable peptide moiety, for example, a linker comprising structural formula (IVa), (IVb), (IVc), or (IVd):

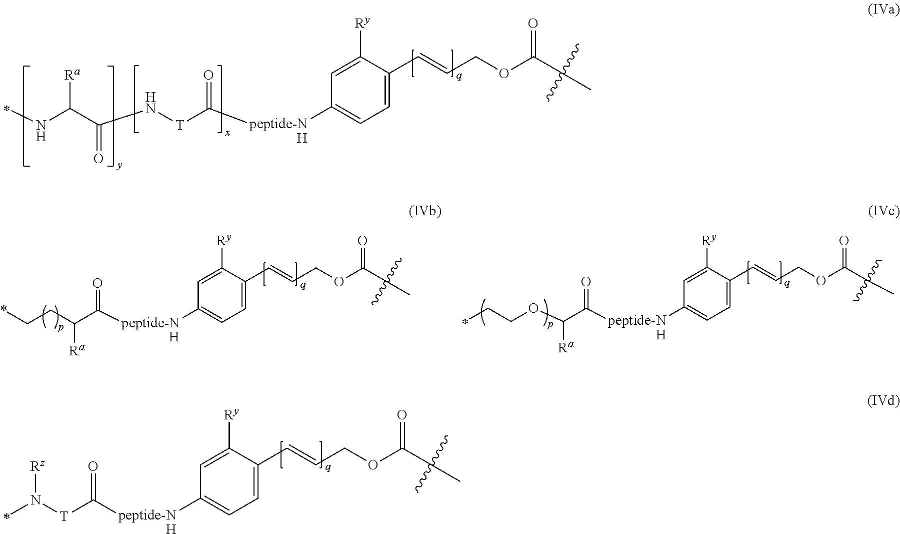

or a pharmaceutically acceptable salt thereof, wherein:

peptide represents a peptide (illustrated N→C, wherein peptide includes the amino and carboxy “termini”) a cleavable by a lysosomal enzyme;

T represents a polymer comprising one or more ethylene glycol units or an alkylene chain, or combinations thereof:

R^{a} is selected from hydrogen, C₁₋₆alkyl, SO₃H and CH₂SO₃H;

R^{y} is hydrogen or C₁₋₄ alkyl-(O)ᵣ—(C₁₋₄ alkylene)ₛ-G¹ or C₁₋₄ alkyl-(N)—[(C₁₋₄ alkylene)-G¹]₂;

R^{z} is CM alkyl-(O)ᵣ—(C₁₋₄ alkylene)ₛ-G²;

G¹ is SO₃H, CO₂H, PEG 4-32, or sugar moiety;

G² is SO₃H, CO₂H, or PEG 4-32 moiety;

r is 0 or 1;

s is 0 or 1;

p is an integer ranging from 0 to 5;

q is 0 or 1:

x is 0 or 1;

y is 0 or 1;

[symbol] represents the point of attachment of the linker to the Bcl-xL inhibitor; and

* represents the point of attachment to the remainder of the linker.

In certain embodiments, the linker comprises an enzymatically cleavable peptide moiety, for example, a linker comprising structural formula (IVa), (IVb), (IVc), (IVd) or a pharmaceutically acceptable salt thereof.

In certain embodiments, linker L comprises a segment according to structural formula IVa or IVb or a pharmaceutically acceptable salt thereof.

In certain embodiments, the peptide is selected from a tripeptide or a dipeptide. In particular embodiments, the dipeptide is selected from: Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser, Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit, or a pharmaceutically acceptable salt thereof.

Exemplary embodiments of linkers according to structural formula (IVa) that may be included in the ADCs described herein include the linkers illustrated below (as illustrated, the linkers include a group suitable for covalently linking the linker to an antibody):

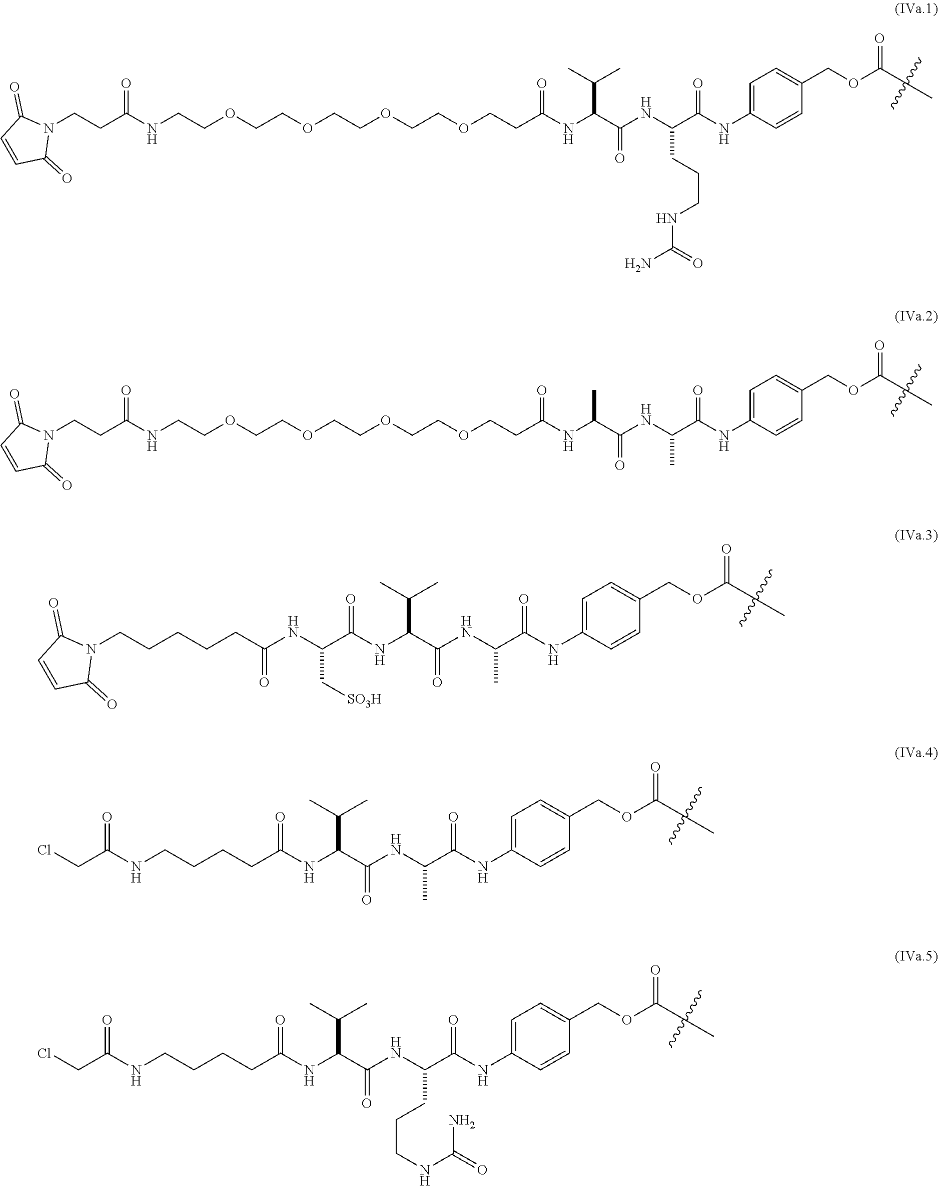 [SMILES: *C(=O)OCc1ccc(NC(=O)[C@H](CCCNC(N)=O)NC(=O)[C@@H](NC(=O)CCOCCOCCOCCOCCNC(=O)CCN2C(=O)C=CC2=O)C(C)C)cc1 ; *C(=O)OCc1ccc(NC(=O)[C@H](C)NC(=O)[C@H](C)NC(=O)CCOCCOCCOCCOCCNC(=O)CCN2C(=O)C=CC2=O)cc1 ; *C(=O)OCc1ccc(NC(=O)[C@H](C)NC(=O)[C@@H](NC(=O)[C@H](CS(=O)(=O)O)NC(=O)CCCCCN2C(=O)C=CC2=O)C(C)C)cc1 ; *C(=O)OCc1ccc(NC(=O)[C@H](C)NC(=O)[C@@H](NC(=O)CCCCNC(=O)CCl)C(C)C)cc1 ; *C(=O)OCc1ccc(NC(=O)[C@H](CCCNC(N)=O)NC(=O)[C@@H](NC(=O)CCCCNC(=O)CCl)C(C)C)cc1]

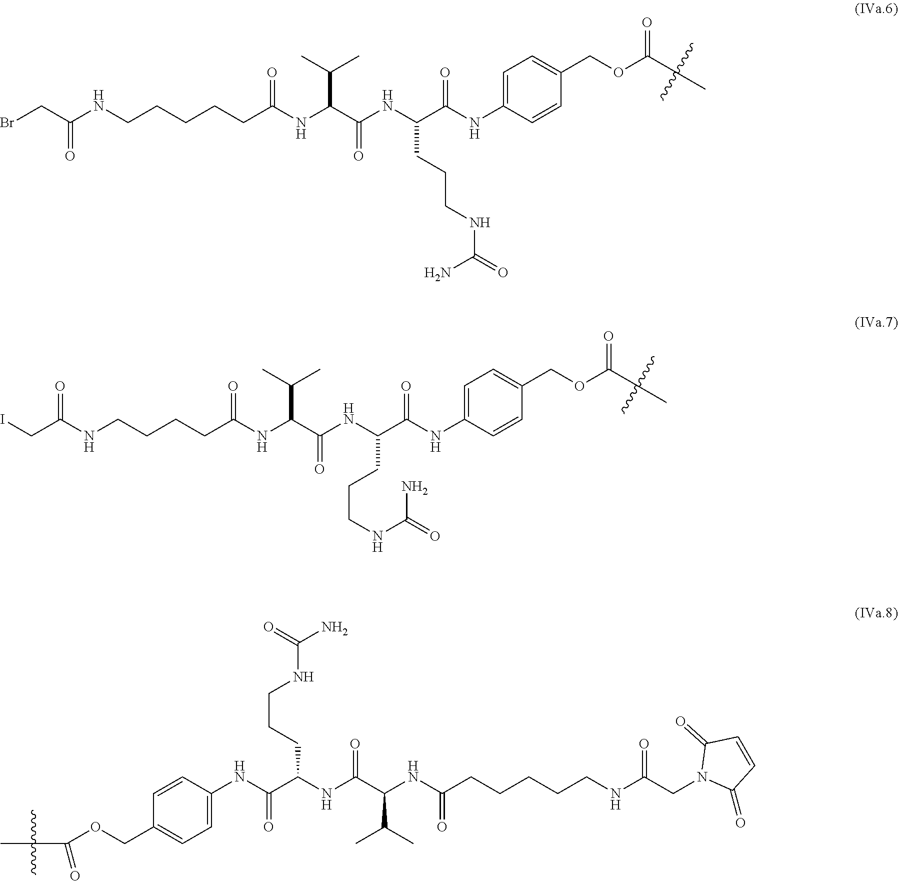 [SMILES: *C(=O)OCc1ccc(NC(=O)[C@H](CCCNC(N)=O)NC(=O)[C@@H](NC(=O)CCCCCNC(=O)CBr)C(C)C)cc1 ; *C(=O)OCc1ccc(NC(=O)[C@H](CCCNC(N)=O)NC(=O)[C@@H](NC(=O)CCCCNC(=O)CI)C(C)C)cc1 ; *C(=O)OCc1ccc(NC(=O)[C@H](CCCNC(N)=O)NC(=O)[C@@H](NC(=O)CCCCCNC(=O)CN2C(=O)C=CC2=O)C(C)C)cc1]

Exemplary embodiments of linkers according to structural formula (IVb), (IVc), or (IVd) that may be included in the ADCs described herein include the linkers illustrated below (as illustrated, the linkers include a group suitable for covalently linking the linker to an antibody):

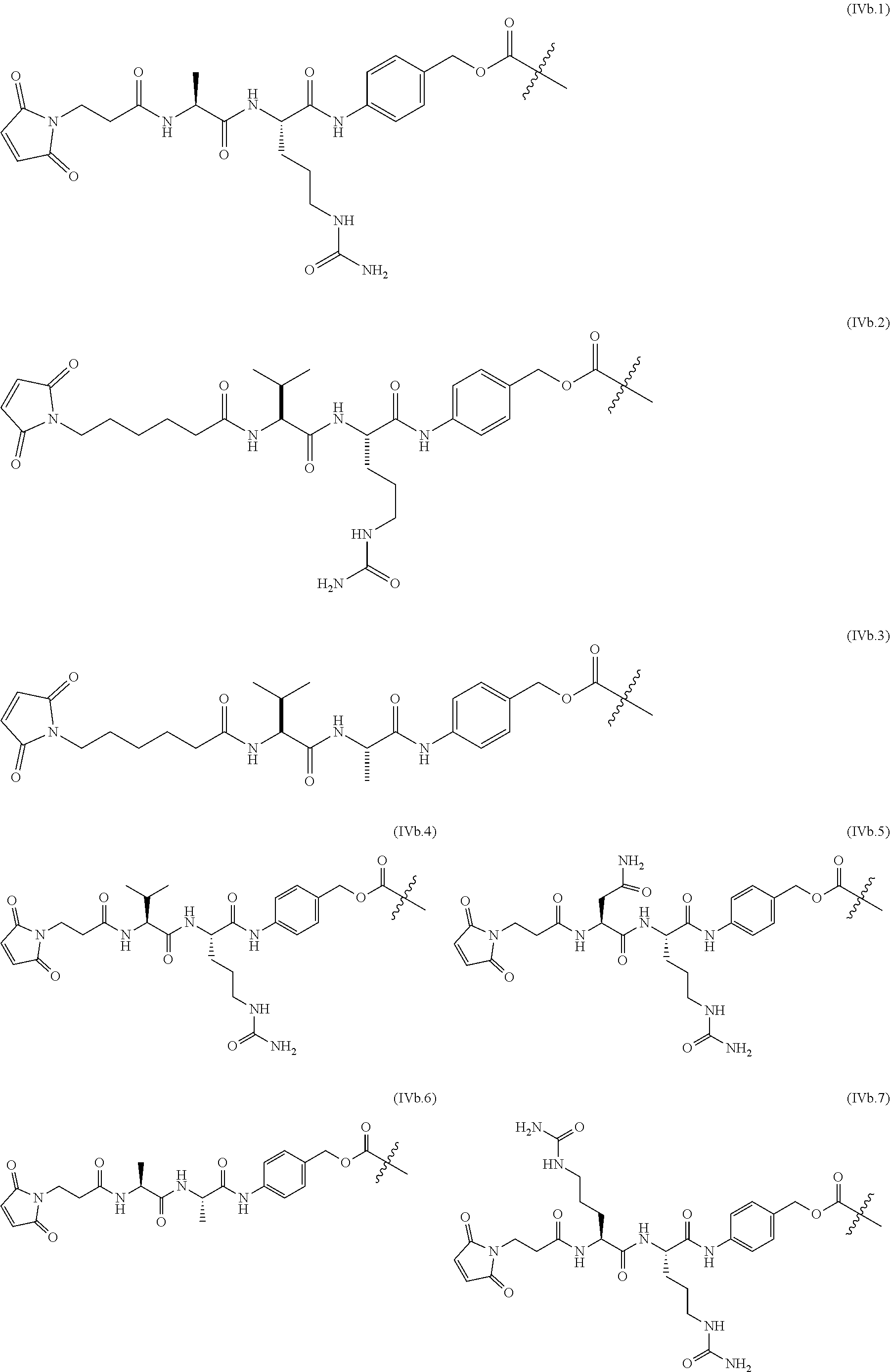

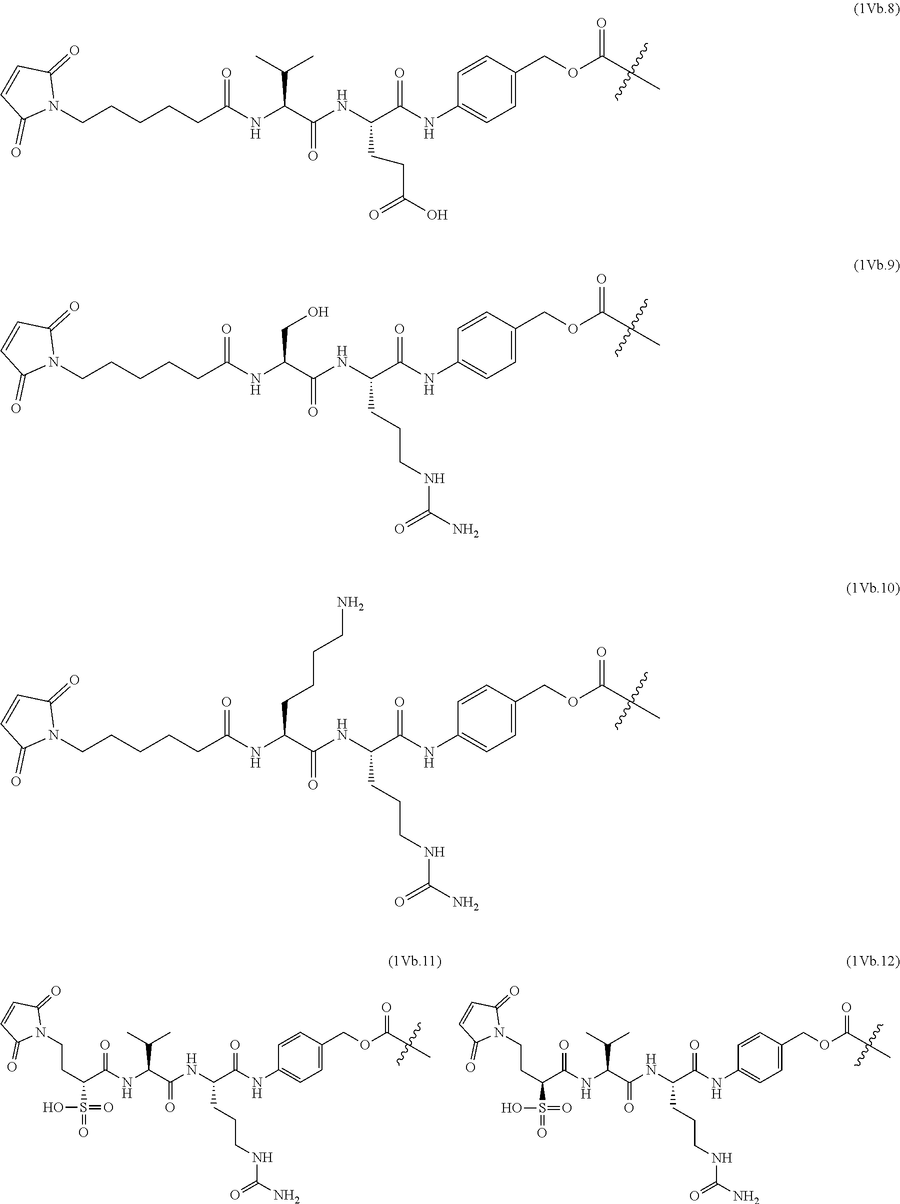 [SMILES: *C(=O)OCc1ccc(NC(=O)[C@H](CCC(=O)O)NC(=O)[C@@H](NC(=O)CCCCCN2C(=O)C=CC2=O)C(C)C)cc1 ; *C(=O)OCc1ccc(NC(=O)[C@H](CCCNC(N)=O)NC(=O)[C@H](CO)NC(=O)CCCCCN2C(=O)C=CC2=O)cc1 ; *C(=O)OCc1ccc(NC(=O)[C@H](CCCNC(N)=O)NC(=O)[C@H](CCCCN)NC(=O)CCCCCN2C(=O)C=CC2=O)cc1 ; *C(=O)OCc1ccc(NC(=O)[C@H](CCCNC(N)=O)NC(=O)[C@@H](NC(=O)[C@@H](CCN2C(=O)C=CC2=O)S(=O)(=O)O)C(C)C)cc1 ; *C(=O)OCc1ccc(NC(=O)[C@H](CCCNC(N)=O)NC(=O)[C@@H](NC(=O)[C@H](CCN2C(=O)C=CC2=O)S(=O)(=O)O)C(C)C)cc1]

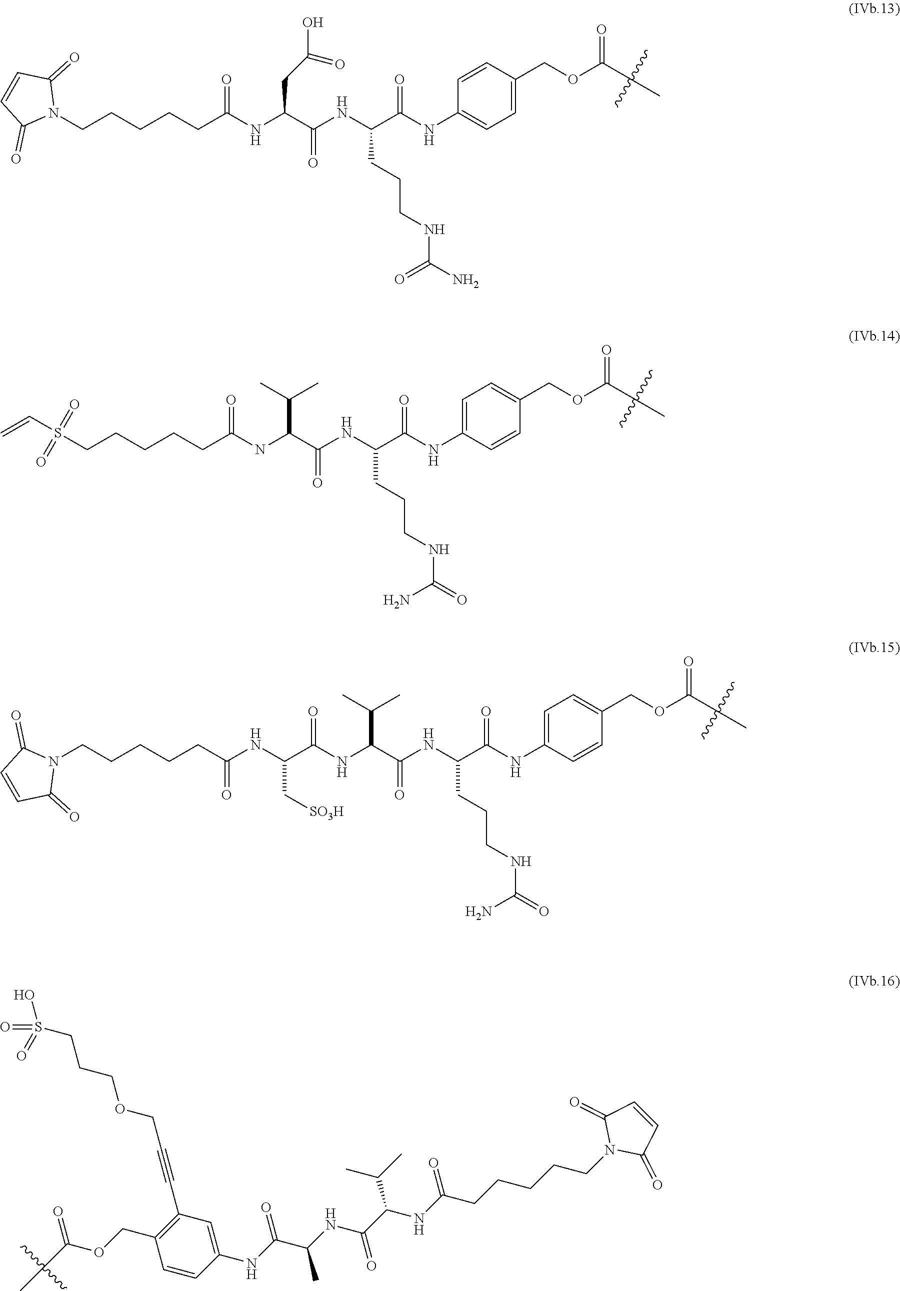 [SMILES: *C(=O)OCc1ccc(NC(=O)[C@H](CCCNC(N)=O)NC(=O)[C@H](CC(=O)O)NC(=O)CCCCCN2C(=O)C=CC2=O)cc1 ; *C(=O)OCc1ccc(NC(=O)[C@H](CCCNC(N)=O)NC(=O)[C@@H](NC(=O)CCCCCS(=O)(=O)C=C)C(C)C)cc1 ; *C(=O)OCc1ccc(NC(=O)[C@H](CCCNC(N)=O)NC(=O)[C@@H](NC(=O)[C@H](CS(=O)(=O)O)NC(=O)CCCCCN2C(=O)C=CC2=O)C(C)C)cc1 ; *C(=O)OCc1ccc(NC(=O)[C@H](C)NC(=O)[C@@H](NC(=O)CCCCCN2C(=O)C=CC2=O)C(C)C)cc1C#CCOCCCS(=O)(=O)O]

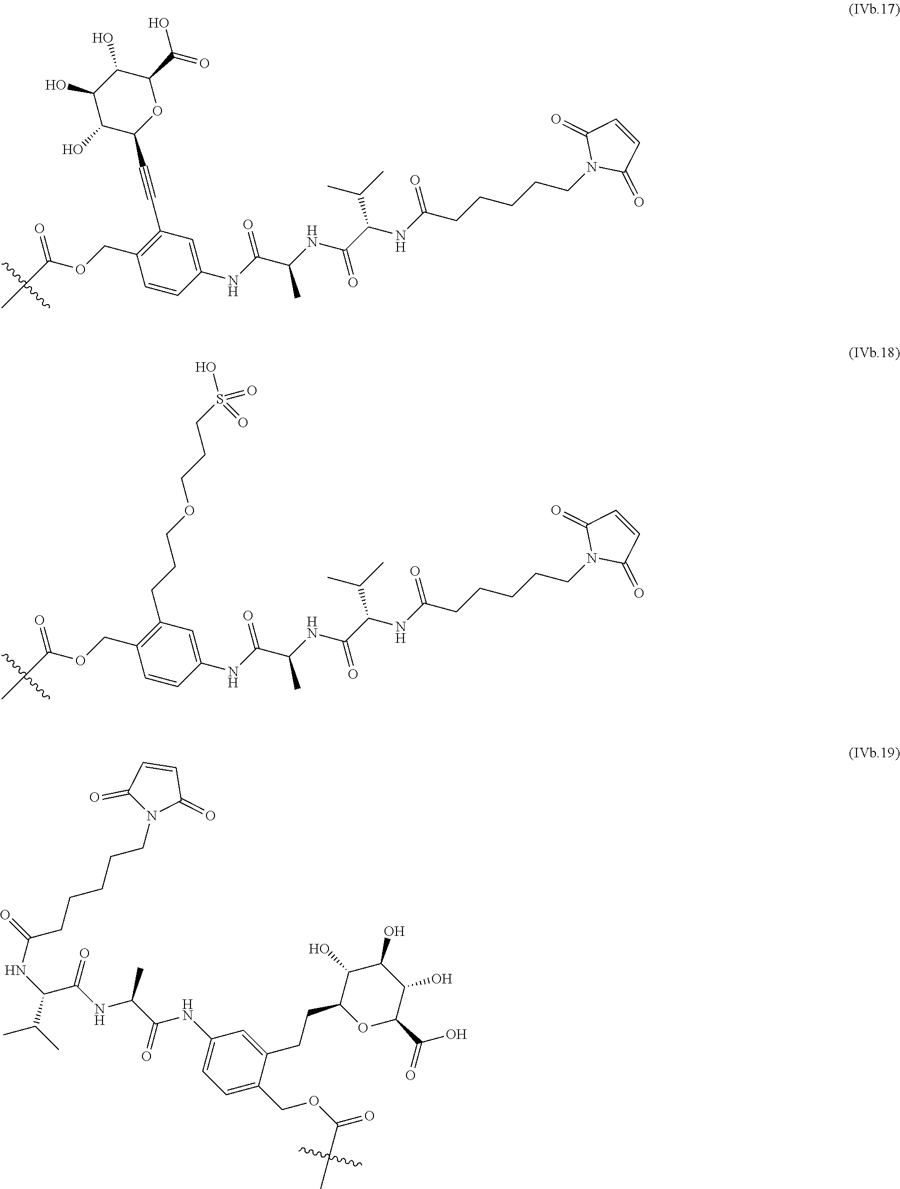 [SMILES: *C(=O)OCc1ccc(NC(=O)[C@H](C)NC(=O)[C@@H](NC(=O)CCCCCN2C(=O)C=CC2=O)C(C)C)cc1C#C[C@@H]1O[C@H](C(=O)O)[C@@H](O)[C@H](O)[C@H]1O ; *C(=O)OCc1ccc(NC(=O)[C@H](C)NC(=O)[C@@H](NC(=O)CCCCCN2C(=O)C=CC2=O)C(C)C)cc1CCCOCCCS(=O)(=O)O ; *C(=O)OCc1ccc(NC(=O)[C@H](C)NC(=O)[C@@H](NC(=O)CCCCCN2C(=O)C=CC2=O)C(C)C)cc1CC[C@@H]1O[C@H](C(=O)O)[C@@H](O)[C@H](O)[C@H]1O]

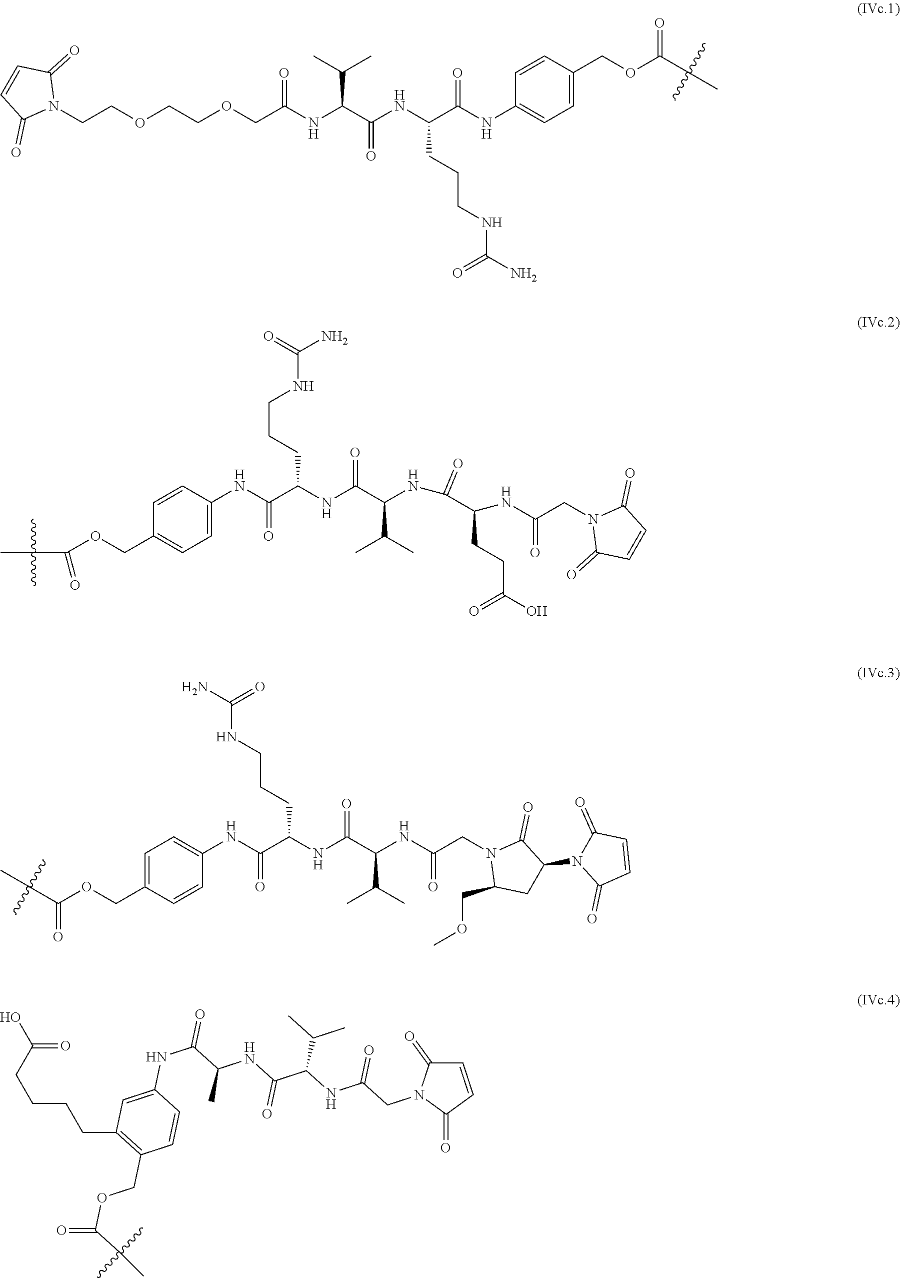 [SMILES: *C(=O)OCc1ccc(NC(=O)[C@H](CCCNC(N)=O)NC(=O)[C@@H](NC(=O)COCCOCCN2C(=O)C=CC2=O)C(C)C)cc1 ; *C(=O)OCc1ccc(NC(=O)[C@H](CCCNC(N)=O)NC(=O)[C@@H](NC(=O)[C@H](CCC(=O)O)NC(=O)CN2C(=O)C=CC2=O)C(C)C)cc1 ; *C(=O)OCc1ccc(NC(=O)[C@H](CCCNC(N)=O)NC(=O)[C@@H](NC(=O)CN2C(=O)[C@@H](N3C(=O)C=CC3=O)C[C@H]2COC)C(C)C)cc1 ; *C(=O)OCc1ccc(NC(=O)[C@H](C)NC(=O)[C@@H](NC(=O)CN2C(=O)C=CC2=O)C(C)C)cc1CCCCC(=O)O]

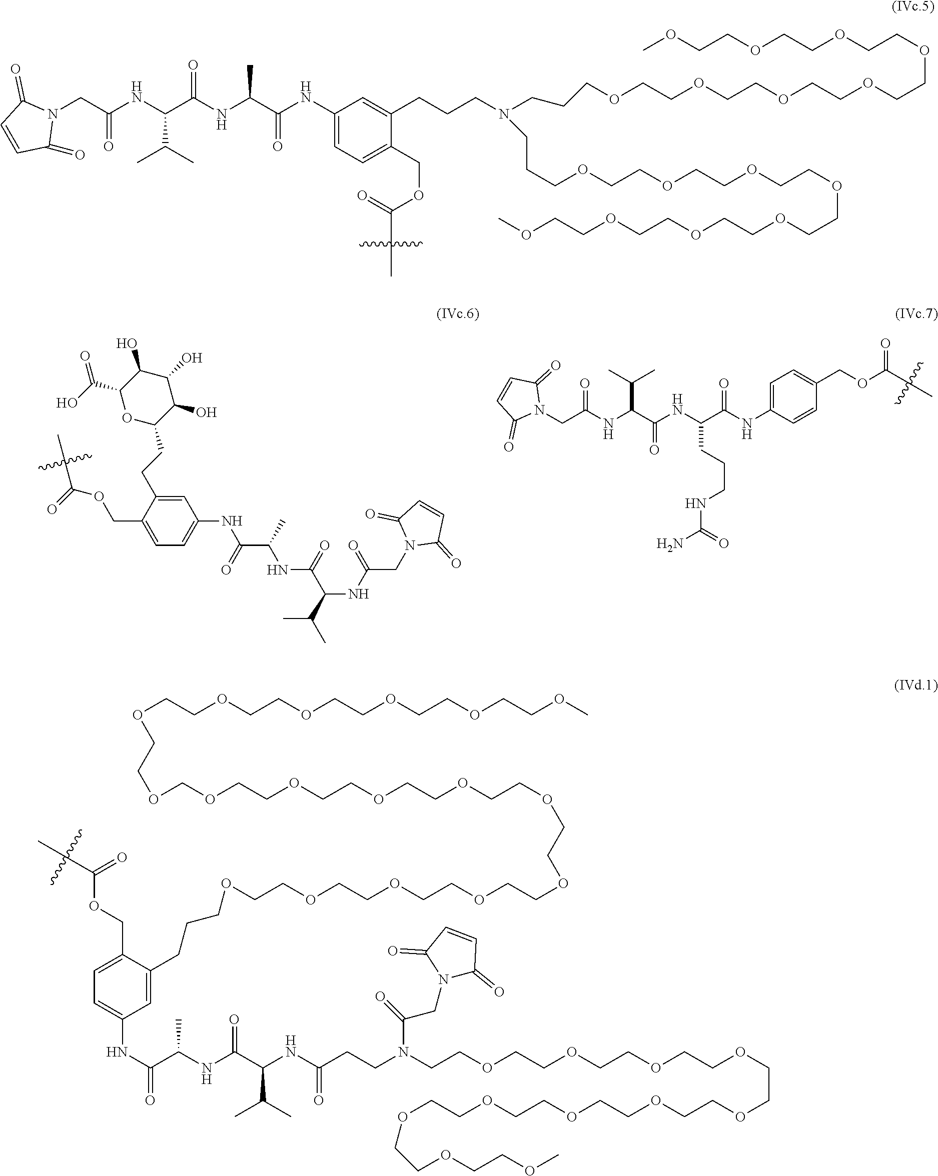

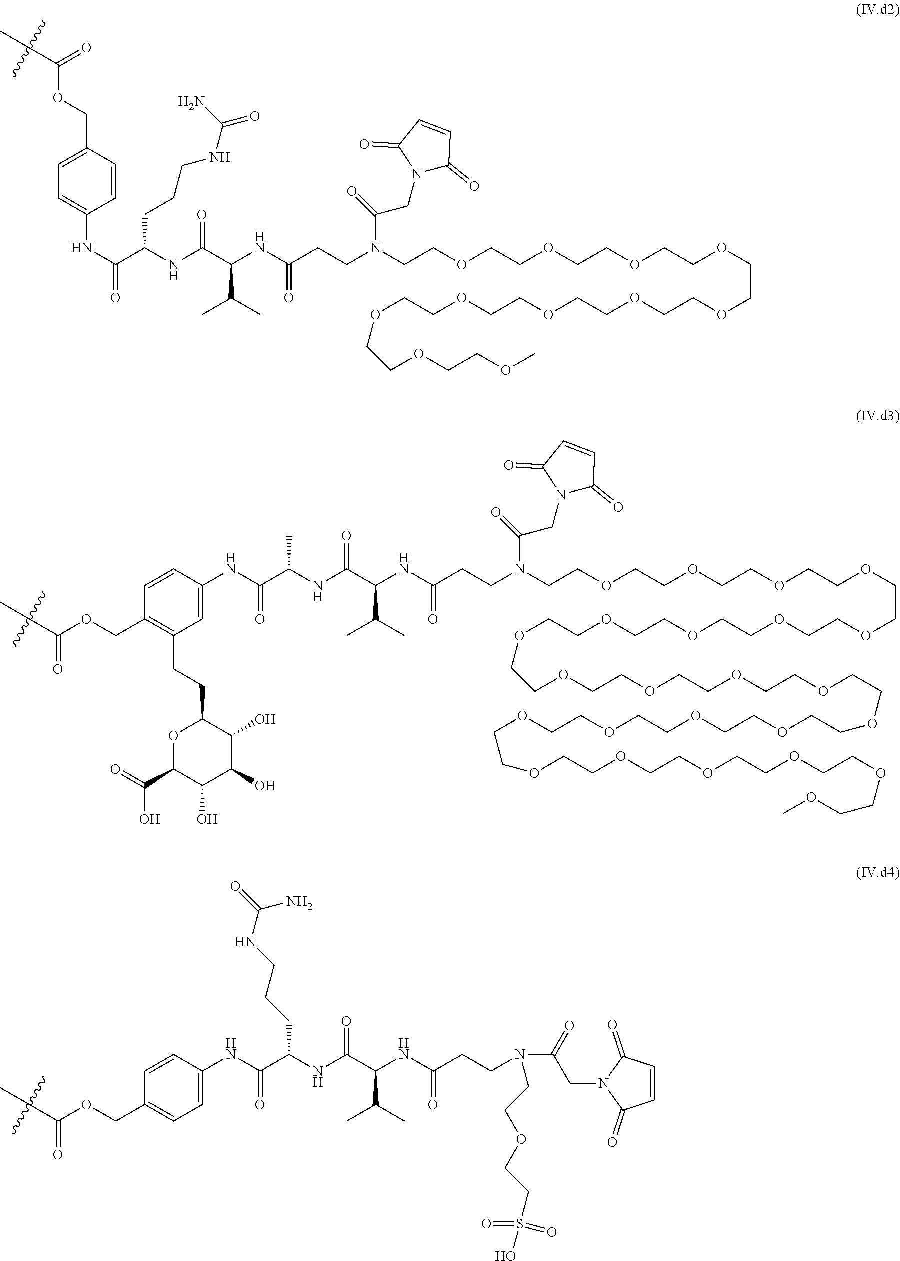 [SMILES: *C(=O)OCc1ccc(NC(=O)[C@H](CCCNC(N)=O)NC(=O)[C@@H](NC(=O)CCN(CCOCCOCCOCCOCCOCCOCCOCCOCCOCCOCCOC)C(=O)CN2C(=O)C=CC2=O)C(C)C)cc1 ; *C(=O)OCc1ccc(NC(=O)[C@H](C)NC(=O)[C@@H](NC(=O)CCN(CCOCCOCCOCCOCCOCCOCCOCCOCCOCCOCCOCCOCCOCCOCCOCCOCCOCCOCCOCCOCCOCCOCCOCCOC)C(=O)CN2C(=O)C=CC2=O)C(C)C)cc1CC[C@@H]1O[C@H](C(=O)O)[C@@H](O)[C@H](O)[C@H]1O ; *C(=O)OCc1ccc(NC(=O)[C@H](CCCNC(N)=O)NC(=O)[C@@H](NC(=O)CCN(CCOCCS(=O)(=O)O)C(=O)CN2C(=O)C=CC2=O)C(C)C)cc1]

In certain embodiments, the linker comprises an enzymatically cleavable sugar moiety, for example, a linker comprising structural formula (Va), (Vb), (Vc), (Vd), or (Ve):

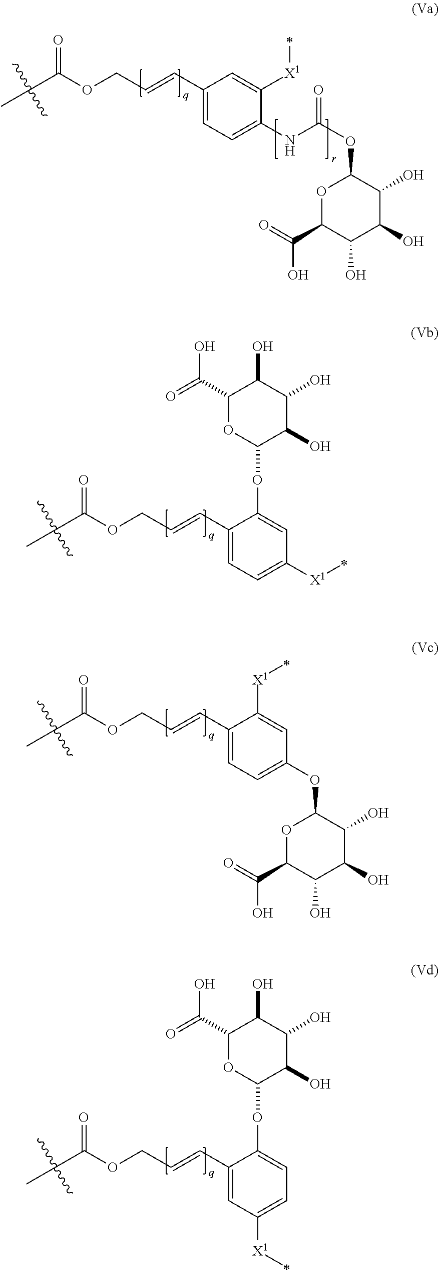

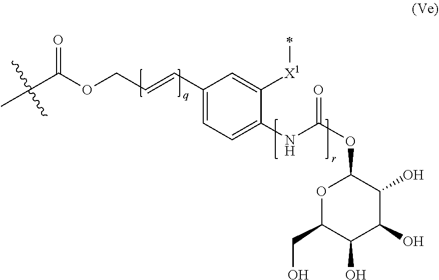

or a pharmaceutically acceptable salt thereof, wherein:
q is 0 or 1;
r is 0 or 1;
X¹ is CH₂, O or NH;
[symbol] represents the point of attachment of the linker to the drug; and
* represents the point of attachment to the remainder of the linker.

Exemplary embodiments of linkers according to structural formula (Va) that may be included in the ADCs described herein include the linkers illustrated below (as illustrated, the linkers include a group suitable for covalently linking the linker to an anti-CD98 antibody):

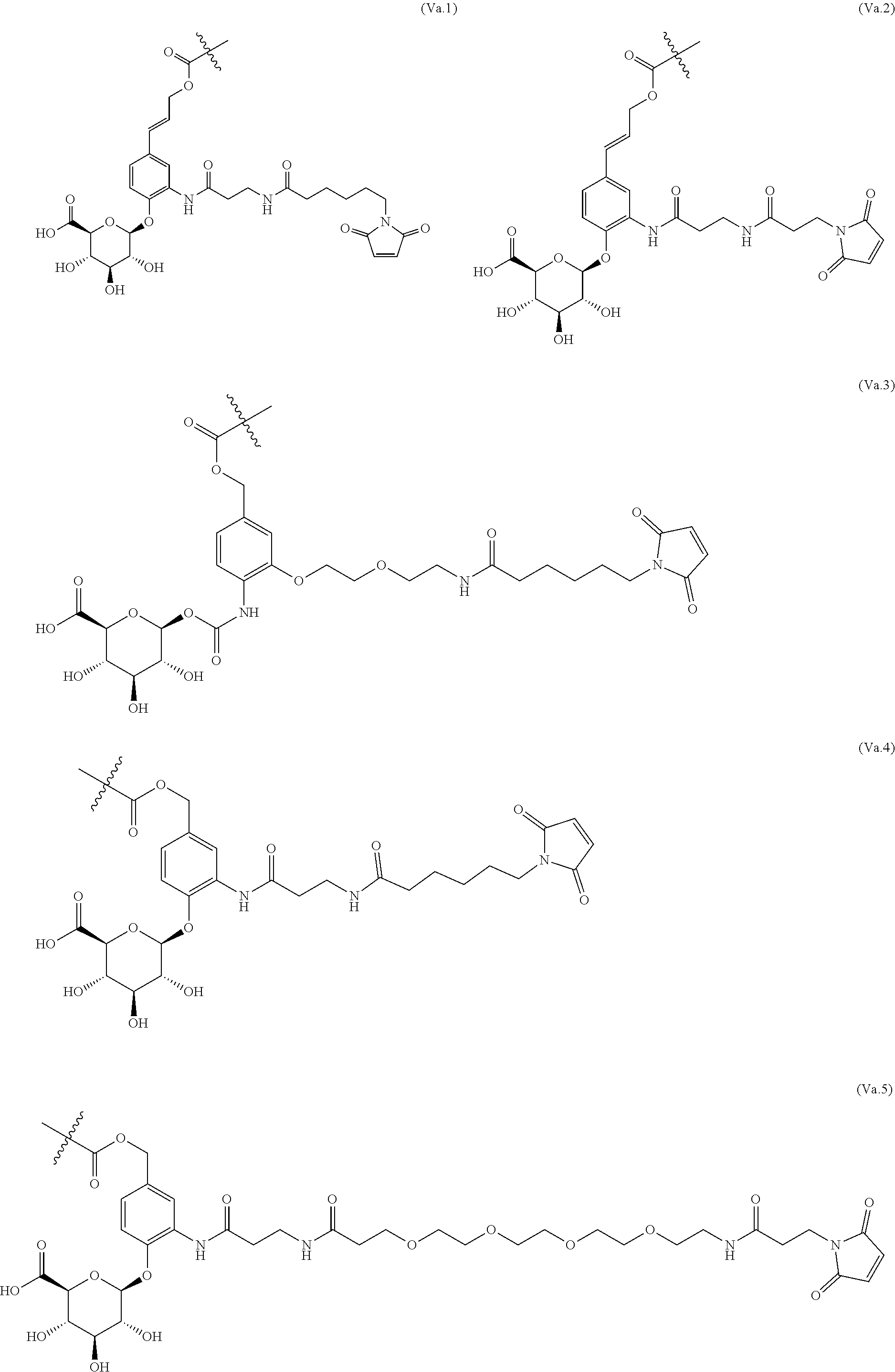

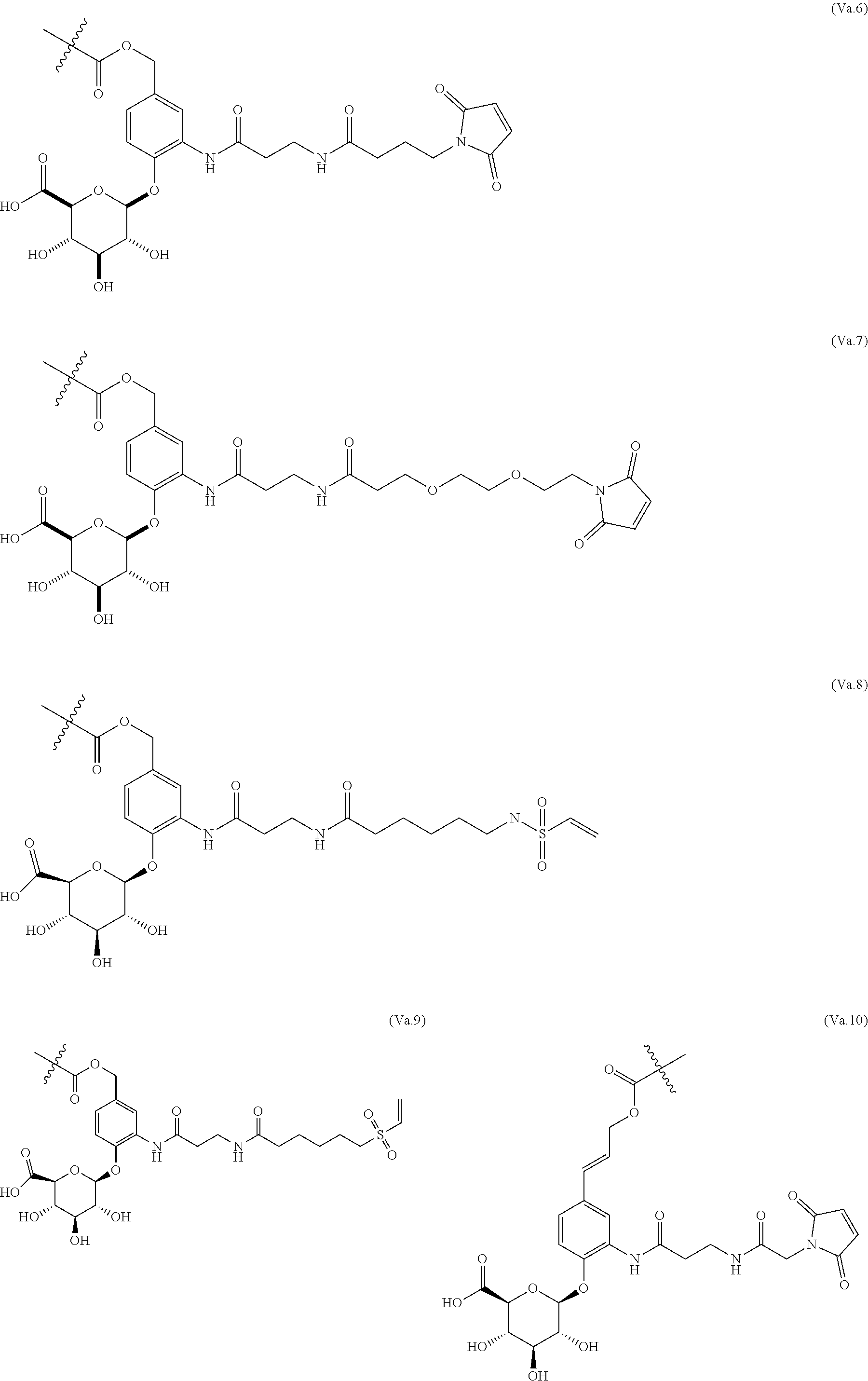

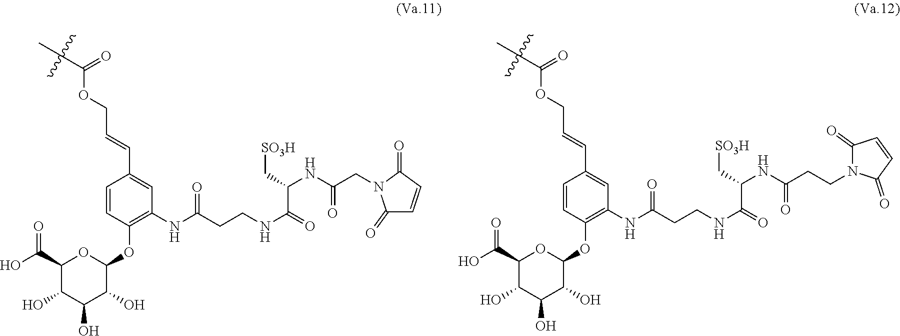 [SMILES: *C(=O)OC/C=C/c1ccc(O[C@@H]2O[C@H](C(=O)O)[C@@H](O)[C@H](O)[C@H]2O)c(NC(=O)CCNC(=O)[C@H](CS(=O)(=O)O)NC(=O)CN2C(=O)C=CC2=O)c1 ; *C(=O)OC/C=C/c1ccc(O[C@@H]2O[C@H](C(=O)O)[C@@H](O)[C@H](O)[C@H]2O)c(NC(=O)CCNC(=O)[C@H](CS(=O)(=O)O)NC(=O)CCN2C(=O)C=CC2=O)c1]

Exemplary embodiments of linkers according to structural formula (Vb) that may be included in the ADCs described herein include the linkers illustrated below (as illustrated, the linkers include a group suitable for covalently linking the linker to an anti-CD98 antibody):

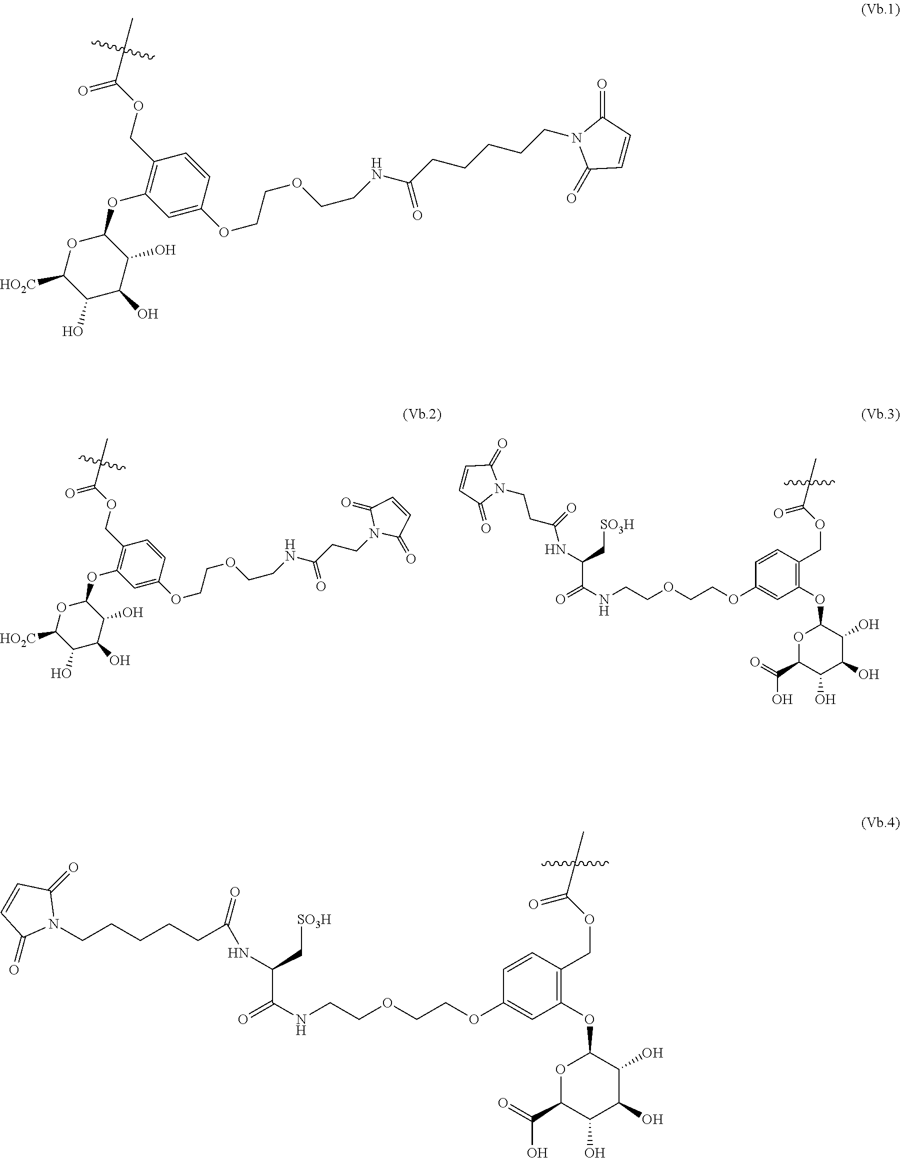 [SMILES: *C(=O)OCc1ccc(OCCOCCNC(=O)CCCCCN2C(=O)C=CC2=O)cc1O[C@@H]1O[C@H](C(=O)O)[C@@H](O)[C@H](O)[C@H]1O ; *C(=O)OCc1ccc(OCCOCCNC(=O)CCN2C(=O)C=CC2=O)cc1O[C@@H]1O[C@H](C(=O)O)[C@@H](O)[C@H](O)[C@H]1O ; *C(=O)OCc1ccc(OCCOCCNC(=O)[C@H](CS(=O)(=O)O)NC(=O)CCN2C(=O)C=CC2=O)cc1O[C@@H]1O[C@H](C(=O)O)[C@@H](O)[C@H](O)[C@H]1O ; *C(=O)OCc1ccc(OCCOCCNC(=O)[C@H](CS(=O)(=O)O)NC(=O)CCCCCN2C(=O)C=CC2=O)cc1O[C@@H]1O[C@H](C(=O)O)[C@@H](O)[C@H](O)[C@H]1O]

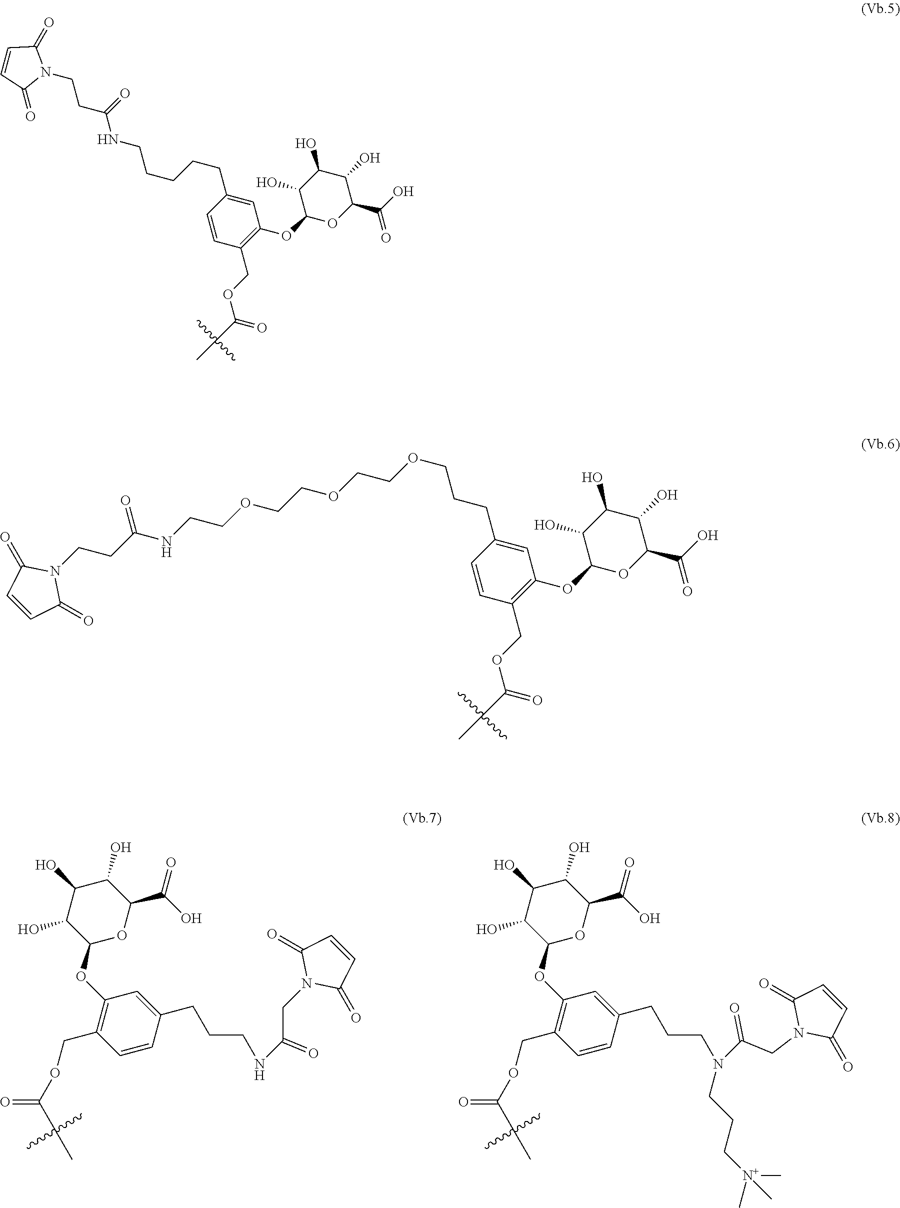 [SMILES: *C(=O)OCc1ccc(CCCCCNC(=O)CCN2C(=O)C=CC2=O)cc1O[C@@H]1O[C@H](C(=O)O)[C@@H](O)[C@H](O)[C@H]1O ; *C(=O)OCc1ccc(CCCOCCOCCOCCNC(=O)CCN2C(=O)C=CC2=O)cc1O[C@@H]1O[C@H](C(=O)O)[C@@H](O)[C@H](O)[C@H]1O ; *C(=O)OCc1ccc(CCCNC(=O)CN2C(=O)C=CC2=O)cc1O[C@@H]1O[C@H](C(=O)O)[C@@H](O)[C@H](O)[C@H]1O ; *C(=O)OCc1ccc(CCCN(CCC[N+](C)(C)C)C(=O)CN2C(=O)C=CC2=O)cc1O[C@@H]1O[C@H](C(=O)O)[C@@H](O)[C@H](O)[C@H]1O]

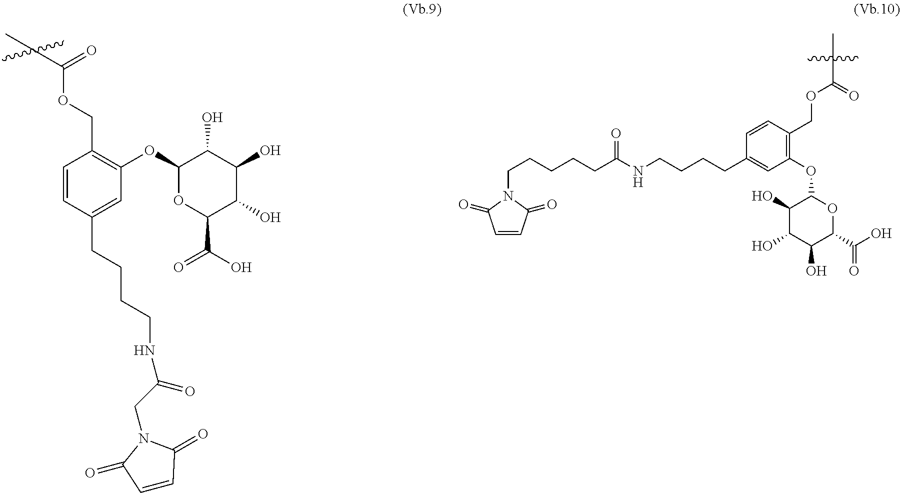

Exemplary embodiments of linkers according to structural formula (Vc) that may be included in the ADCs described herein include the linkers illustrated below (as illustrated, the linkers include a group suitable for covalently linking the linker to an anti-CD98 antibody):

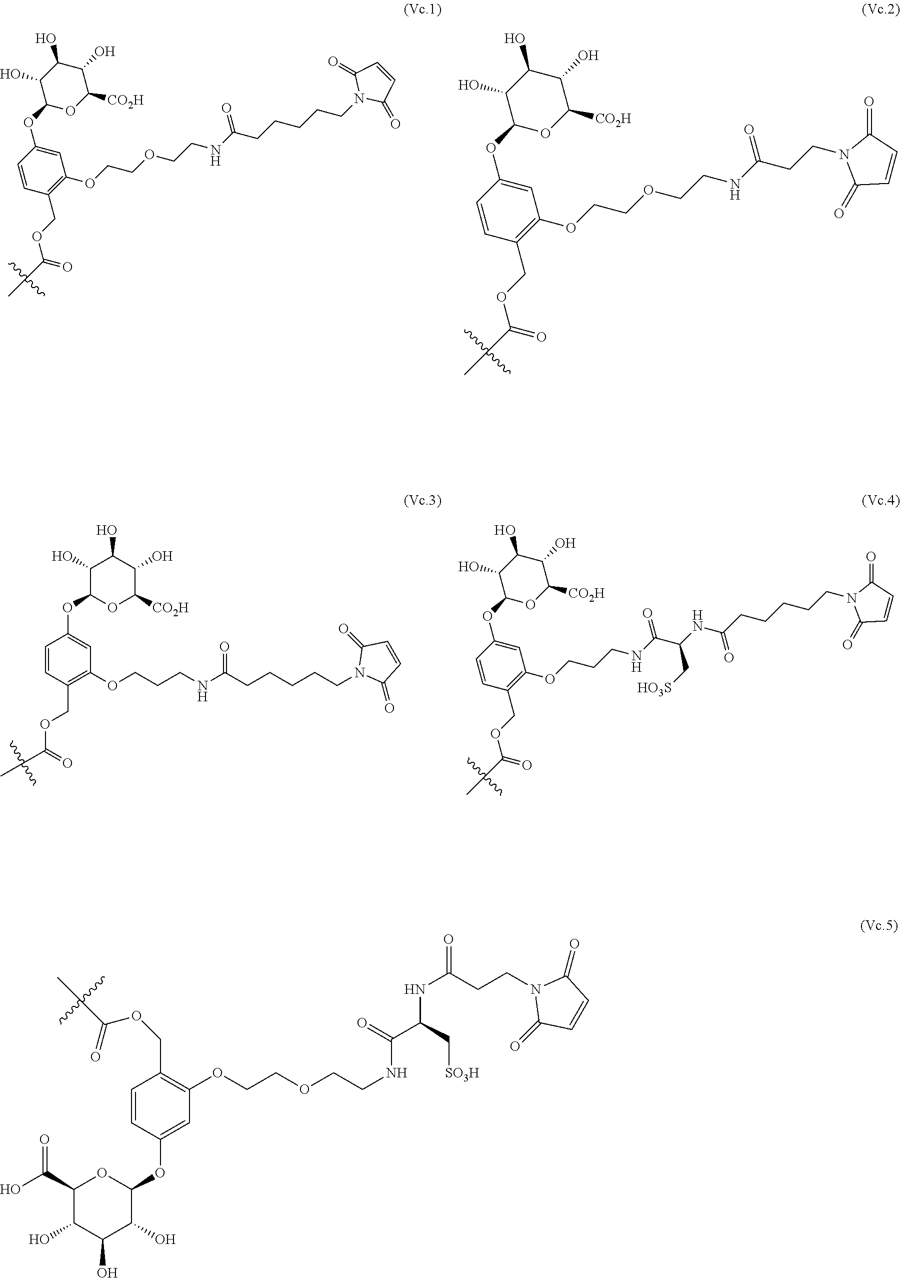

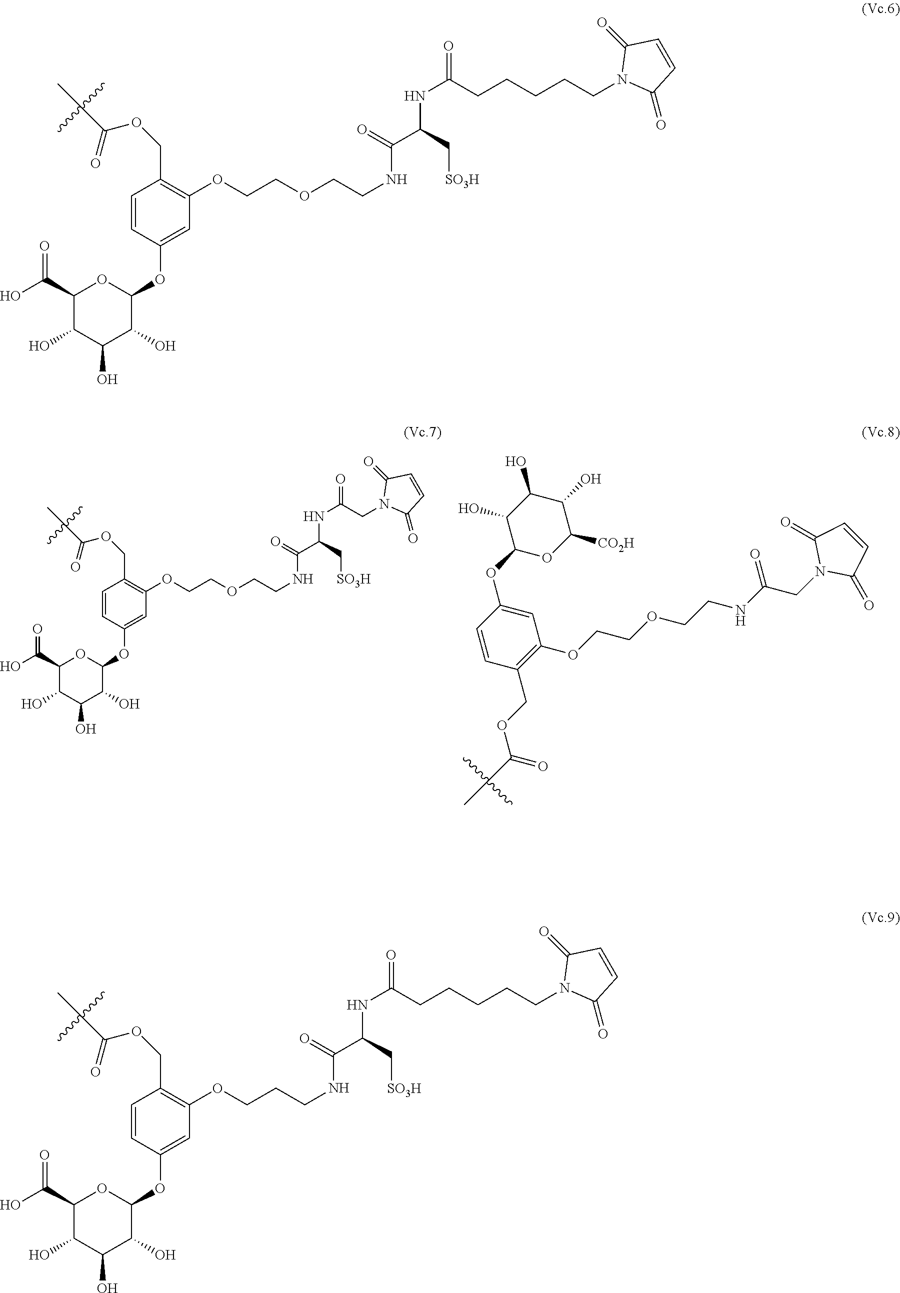 [SMILES: *C(=O)OCc1ccc(O[C@@H]2O[C@H](C(=O)O)[C@@H](O)[C@H](O)[C@H]2O)cc1OCCOCCNC(=O)[C@H](CS(=O)(=O)O)NC(=O)CCCCCN1C(=O)C=CC1=O ; *C(=O)OCc1ccc(O[C@@H]2O[C@H](C(=O)O)[C@@H](O)[C@H](O)[C@H]2O)cc1OCCOCCNC(=O)[C@H](CS(=O)(=O)O)NC(=O)CN1C(=O)C=CC1=O ; *C(=O)OCc1ccc(O[C@@H]2O[C@H](C(=O)O)[C@@H](O)[C@H](O)[C@H]2O)cc1OCCOCCNC(=O)CN1C(=O)C=CC1=O ; *C(=O)OCc1ccc(O[C@@H]2O[C@H](C(=O)O)[C@@H](O)[C@H](O)[C@H]2O)cc1OCCCNC(=O)[C@H](CS(=O)(=O)O)NC(=O)CCCCCN1C(=O)C=CC1=O]

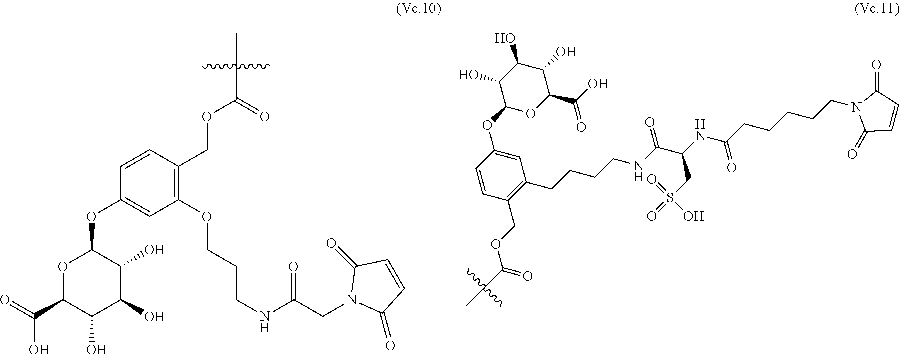 [SMILES: *C(=O)OCc1ccc(O[C@@H]2O[C@H](C(=O)O)[C@@H](O)[C@H](O)[C@H]2O)cc1OCCCNC(=O)CN1C(=O)C=CC1=O ; *C(=O)OCc1ccc(O[C@@H]2O[C@H](C(=O)O)[C@@H](O)[C@H](O)[C@H]2O)cc1CCCCNC(=O)[C@H](CS(=O)(=O)O)NC(=O)CCCCCN1C(=O)C=CC1=O]

Exemplary embodiments of linkers according to structural formula (Vd) that may be included in the ADCs described herein include the linkers illustrated below (as illustrated, the linkers include a group suitable for covalently linking the linker to an anti-CD98 antibody):

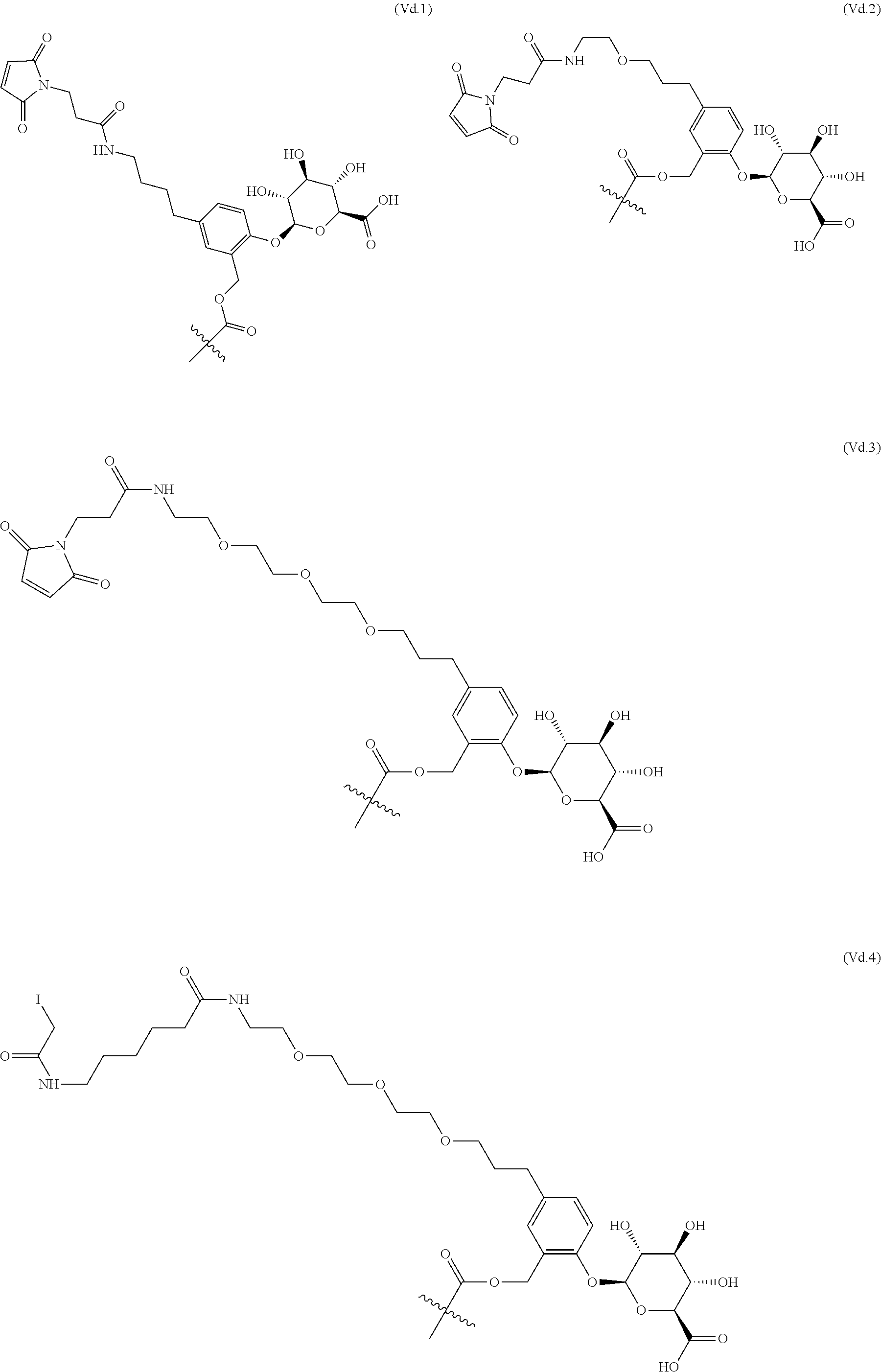 [SMILES: *C(=O)OCc1cc(CCCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCOCCOCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCOCCOCCNC(=O)CCCCCNC(=O)CI)ccc1O[C@@H]1O[C@H](C(=O)O)[C@@H](O)[C@H](O)[C@H]1O]

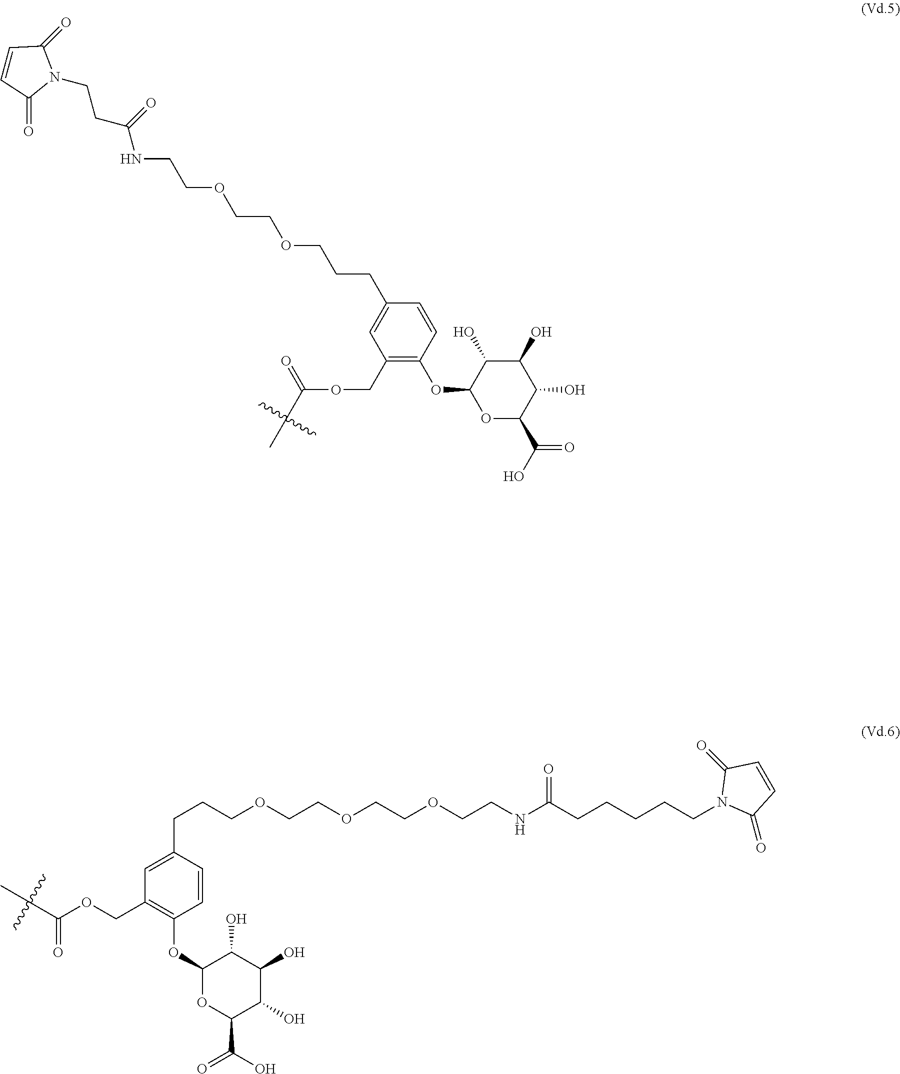 [SMILES: *C(=O)OCc1cc(CCCOCCOCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCOCCOCCNC(=O)CCCCCN2C(=O)C=CC2=O)ccc1O[C@@H]1O[C@H](C(=O)O)[C@@H](O)[C@H](O)[C@H]1O]

Exemplary embodiments of linkers according to structural formula (Ve) that may be included in the ADCs described herein include the linkers illustrated below (as illustrated, the linkers include a group suitable for covalently linking the linker to an anti-CD98 antibody):

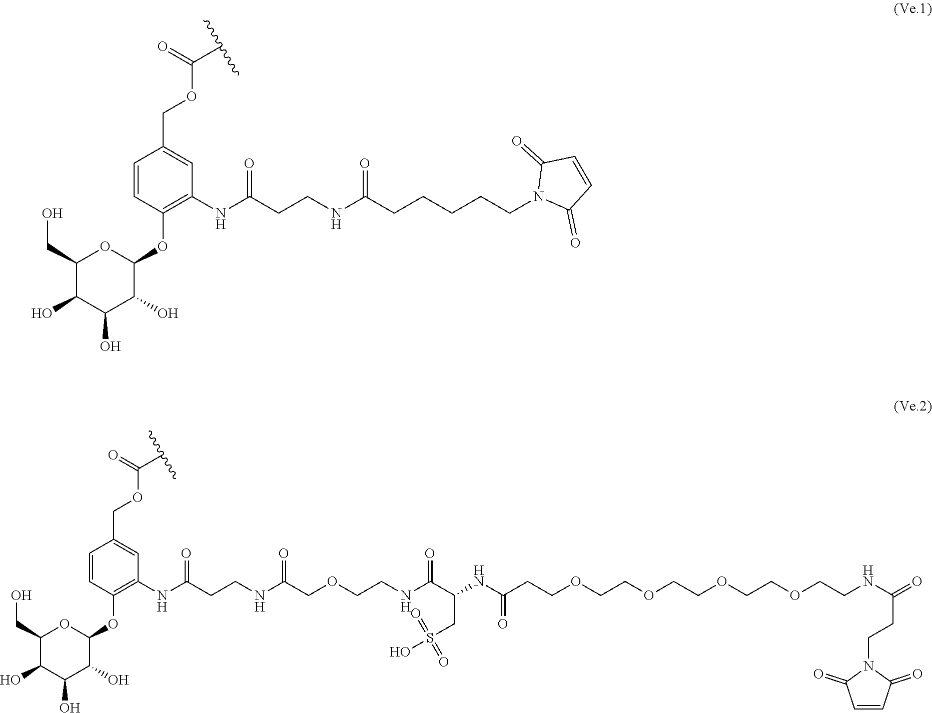 [SMILES: *C(=O)OCc1ccc(O[C@@H]2O[C@H](CO)[C@H](O)[C@H](O)[C@H]2O)c(NC(=O)CCNC(=O)CCCCCN2C(=O)C=CC2=O)c1 ; *C(=O)OCc1ccc(O[C@@H]2O[C@H](CO)[C@H](O)[C@H](O)[C@H]2O)c(NC(=O)CCNC(=O)COCCNC(=O)[C@@H](CS(=O)(=O)O)NC(=O)CCOCCOCCOCCOCCNC(=O)CCN2C(=O)C=CC2=O)c1]

Although cleavable linkers may provide certain advantages, the linkers comprising the ADC described herein need not be cleavable. For noncleavable linkers, the drug release does not depend on the differential properties between the plasma and some cytoplasmic compartments. The release of the drug is postulated to occur after internalization of the ADC via antigen-mediated endocytosis and delivery to lysosomal compartment, where the anti-CD98 antibody is degraded to the level of amino acids through intracellular proteolytic degradation. This process releases a drug derivative, which is formed by the drug, the linker, and the amino acid residue to which the linker was covalently attached. The amino-acid drug metabolites from conjugates with noncleavable linkers are more hydrophilic and generally less membrane permeable, which leads to less bystander effects and less nonspecific toxicides compared to conjugates with a cleavable linker. In general, ADCs with noncleavable linkers have greater stability in circulation than ADCs with cleavable linkers. Non-cleavable linkers may be alkylene chains, or maybe polymeric in natures, such as, for example, based upon polyalkylene glycol polymers, amide polymers, or may include segments of alkylene chains, polyalkylene glycols and/or amide polymers. In certain embodiments, the linker comprises a polyethylene glycol segment having from 1 to 6 ethylene glycol units.

A variety of non-cleavable linkers used to link drugs to antibodies have been described. (See, Jeffrey et al, 2006, Bioconjug. Chem. 17:831-840; Jeffrey et al., 2007, Bioorg. Med Chem. Lett. 17:2278-2280; and Jiang et al, 2005. J. Am. Chem. Soc. 127:11254-11255, the contents of which are incorporated herein by reference). All of these linkers may be included in the ADCs described herein.

In certain embodiments, the linker is non-cleavable in vivo, for example a linker according to structural formula (VIa), (VIb), (VIc) or (VId) (as illustrated, the linkers include a group suitable for covalently linking the linker to an anti-CD98 antibody:

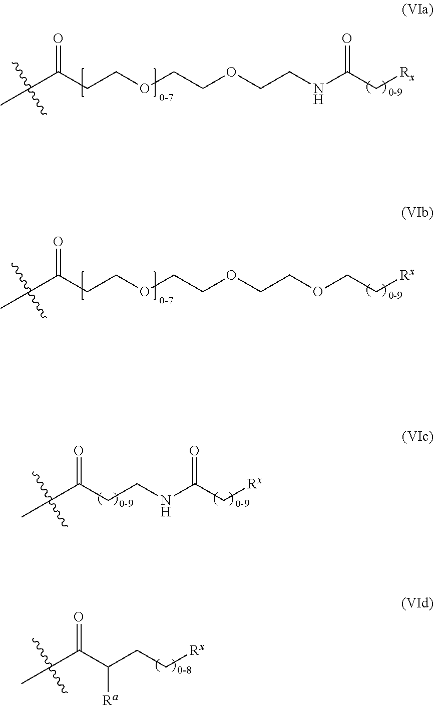

or a pharmaceutically acceptable salt thereof, wherein:

R^{a} is selected from hydrogen, alkyl, sulfonate and methyl sulfonate;

R^{x} is a moiety including a functional group capable of covalently linking the linker to an antibody; and

[symbol] represents the point of attachment of the linker to the Bcl-xL inhibitor.

Exemplary embodiments of linkers according to structural formula (VIa)-(VId) that may be included in the ADCs described herein include the linkers illustrated below (as illustrated, the linkers include a group suitable for covalently linking the linker to an anti-CD98 antibody, and “ [symbol] ” represents the point of attachment to a Bcl-xL inhibitor):

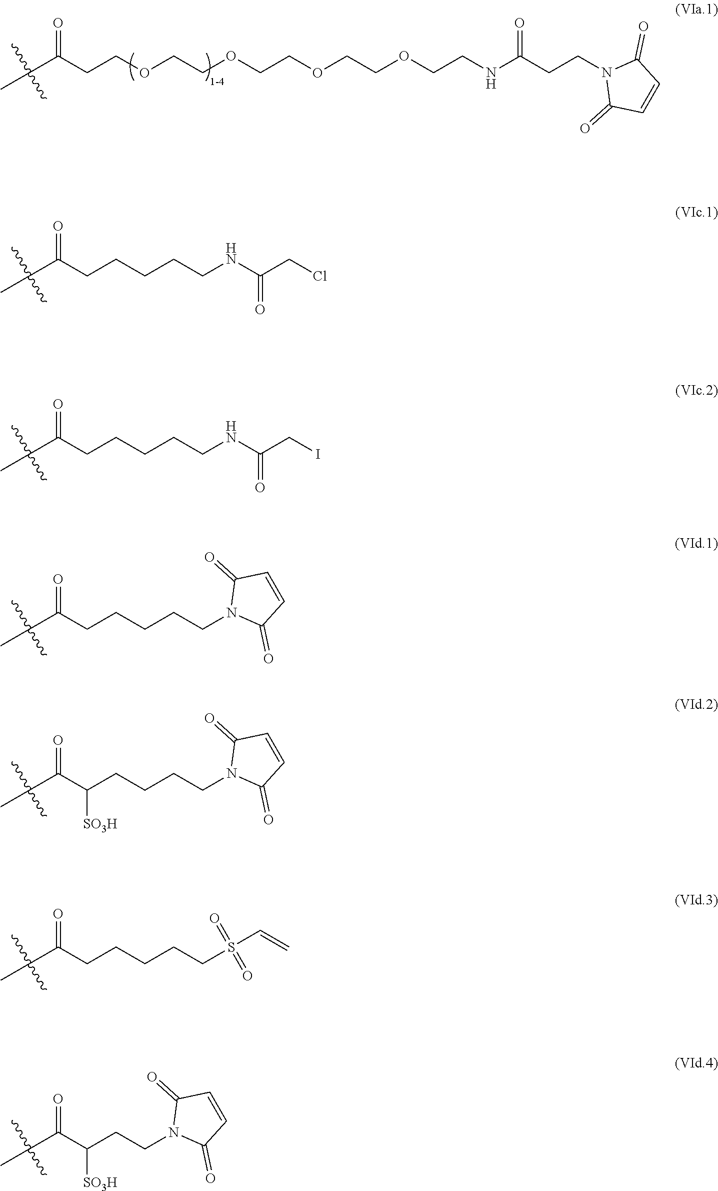

### Groups Used to Attach Linkers to Anti-CD98 Antibodies

Attachment groups can be electrophilic in nature and include: maleimide groups, activated disulfides, active esters such as NHS esters and HOBt esters, haloformates, acid halides, alkyl and benzyl halides such as haloacetamides. As discussed below, there are also emerging technologies related to “self-stabilizing” maleimides and “bridging disulfides” that can be used in accordance with the disclosure.

One example of a “self-stabilizing” maleimide group that hydrolyzes spontaneously under antibody conjugation conditions to give an ADC species with improved stability is depicted in the schematic below. See U S. Published Application No. 2013/0309256, International Application Publication No. WO 2013/173337. Tumey et al, 2014, Bioconjugate Chem. 25: 1871-1880, and Lyon et al., 2014, Nat. Biotechnol. 32: 1059-1062. Thus, the maleimide attachment group is reacted with a sulfhydryl of an antibody to give an intermediate succinimide ring. The hydrolyzed form of the attachment group is resistant to deconjugation in the presence of plasma proteins. Normal system:

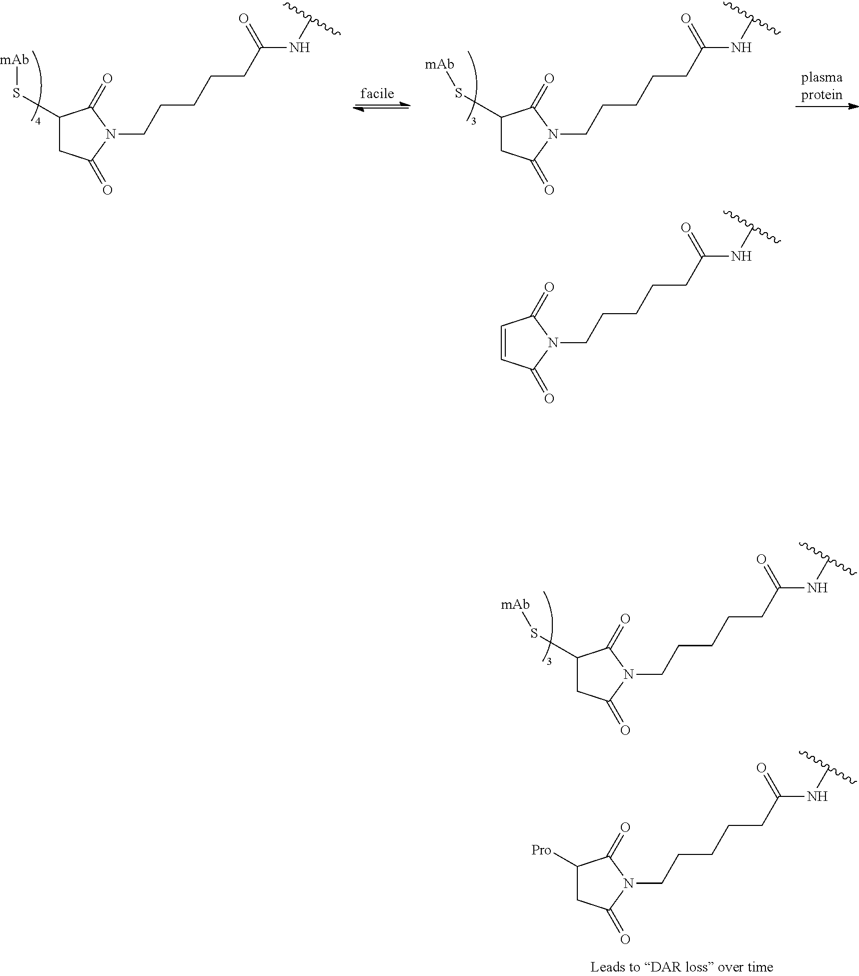

### Self-Stabilizing Attachment

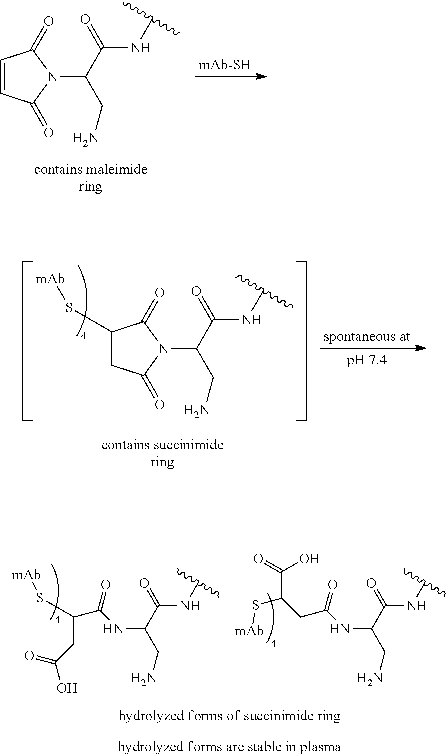

As shown above, the maleimide ring of a linker may react with an antibody Ab, forming a covalent attachment as either a succinimide (closed form) or succinamide (open form).

Polytherics has disclosed a method for bridging a pair of sulfhydryl groups derived from reduction of a native hinge disulfide bond. See, Badescu et al., 2014, Bioconjugate Chem. 25:1124-1136. The reaction is depicted in the schematic below. An advantage of this methodology is the ability to synthesize homogenous DAR4 ADCs by full reduction of IgGs (to give 4 pairs of sulfhydryls) followed by reaction with 4 equivalents of the alkylating agent. ADCs containing “bridged disulfides” are also claimed to have increased stability.

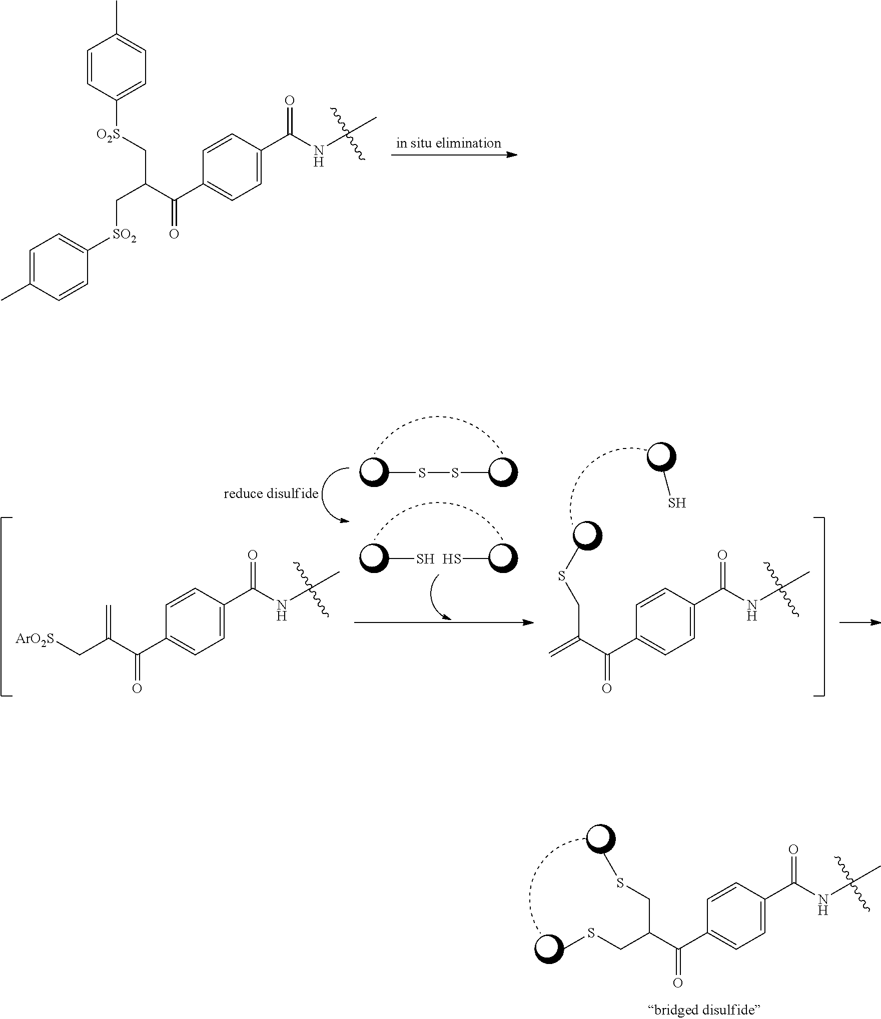

Similarly, as depicted below, a maleimide derivative that is capable of bridging a pair of sulfhydryl groups has been developed. See U S. Published Application No. 2013/0224228.

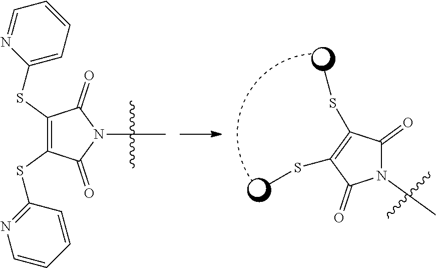 [SMILES: *N1C(=O)C(Sc2ccccn2)=C(Sc2ccccn2)C1=O ; *N1C(=O)C(SC)=C(SC)C1=O]

In certain embodiments the attachment moiety comprises the structural formulae (VIIa), (VIIb), or (VIIc):

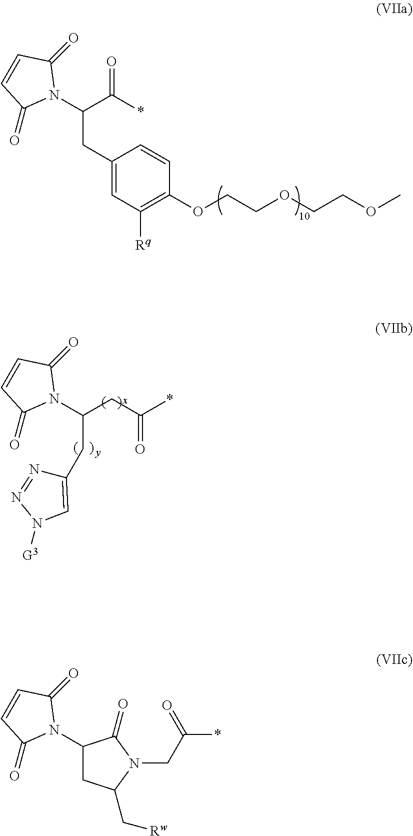

or a pharmaceutically acceptable salt thereof, wherein:

R^{q} is H or —O—(CH₂CH₂O)₁₁—CH₃;

x is 0 or 1;

y is 0 or 1:

G³ is —CH₂CH₂CH₂SO₃H or —CH₂CH₂O—(CH₂CH₂O)₁₁—CH₃;

R^{w} is —O—CH₂CH₂SO₃H or —NH(CO)—CH₂CH₂O—(CH₂CH₂O)₁₂—CH₃; and

* represents the point of attachment to the remainder of the linker.

In certain embodiments, the linker comprises a segment according to structural formulae (VIIIa), (VIIIb), or (VIIIc):

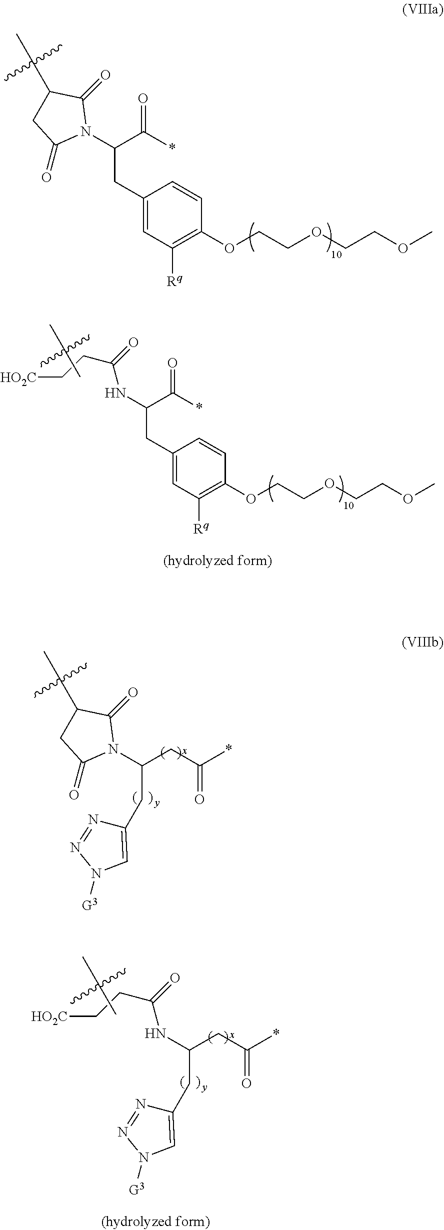

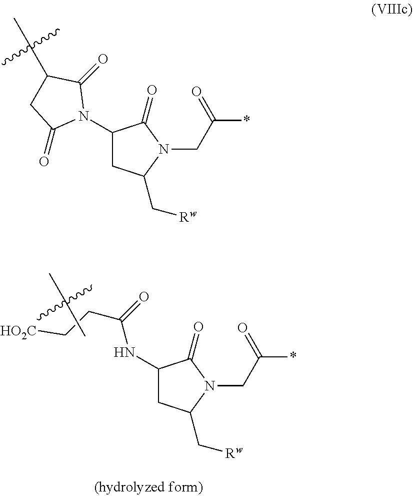

or a hydrolyzed derivative or a pharmaceutically acceptable salt thereof, wherein:

R^{q} is H or —O—(CH₂CH₂O)₁₁—CH₃;

x is 0 or 1;

y is 0 or 1:

G³ is —CH₂CH₂CH₂SO₃H or —CH₂CH₂O—(CH₂CH₂O)₁₁—CH₃;

R^{w} is —O—CH₂CH₂SO₃H or —NH(CO)—CH₂CH₂O—(CH₂CH₂O)₁₂—CH₃;

* represents the point of attachment to the remainder of the linker; and

[symbol] represents the point of attachment of the linker to the antibody, wherein when in the hydrolyzed form, [symbol] can be either at the α-position or β-position of the carboxylic acid next to it.

Exemplary embodiments of linkers according to structural formula (VIIa) and (VIIb) that may be included in the ADCs described herein include the linkers illustrated below (as illustrated, the linkers include a group suitable for covalently linking the linker to an antibody):

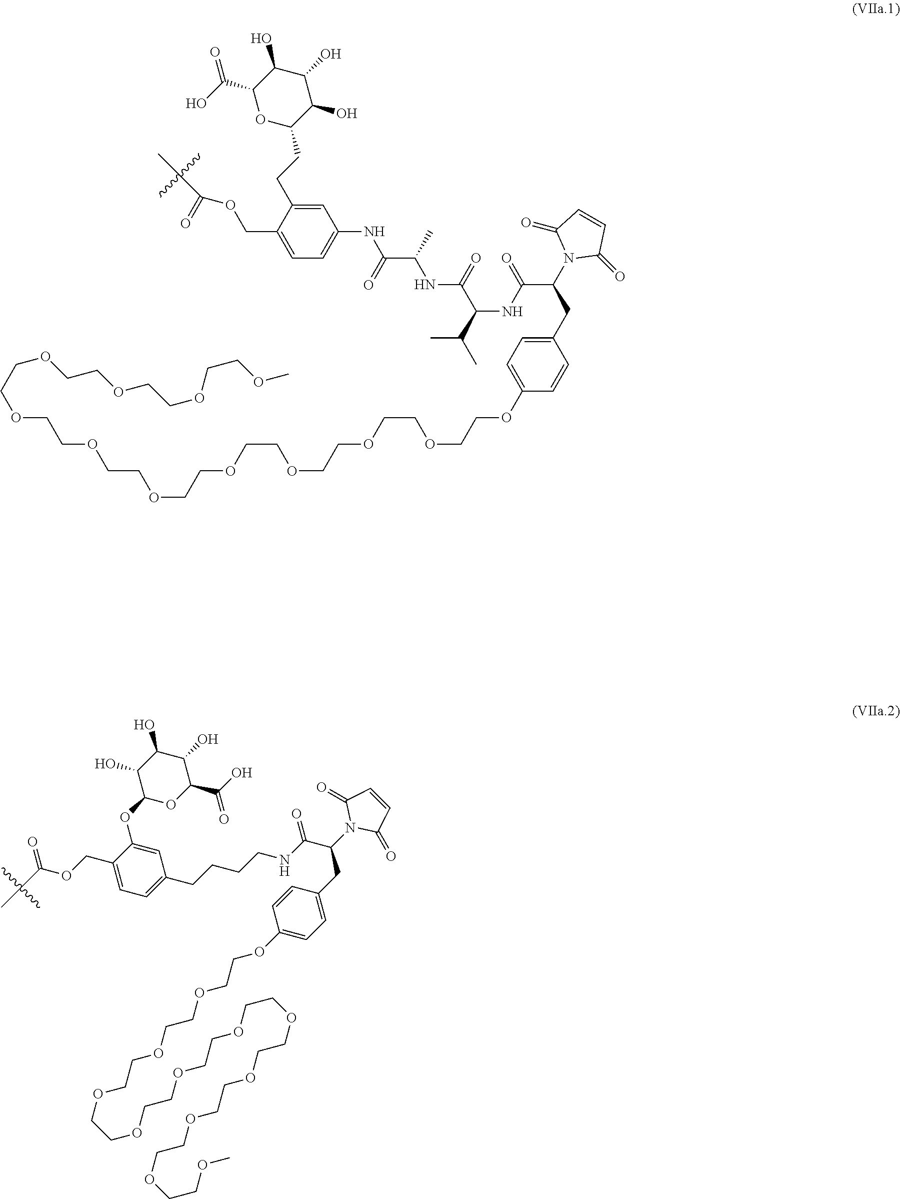 [SMILES: *C(=O)OCc1ccc(NC(=O)[C@H](C)NC(=O)[C@@H](NC(=O)[C@H](Cc2ccc(OCCOCCOCCOCCOCCOCCOCCOCCOCCOCCOCCOC)cc2)N2C(=O)C=CC2=O)C(C)C)cc1CC[C@@H]1O[C@H](C(=O)O)[C@@H](O)[C@H](O)[C@H]1O ; *C(=O)OCc1ccc(CCCCNC(=O)[C@H](Cc2ccc(OCCOCCOCCOCCOCCOCCOCCOCCOCCOCCOCCOC)cc2)N2C(=O)C=CC2=O)cc1O[C@@H]1O[C@H](C(=O)O)[C@@H](O)[C@H](O)[C@H]1O]

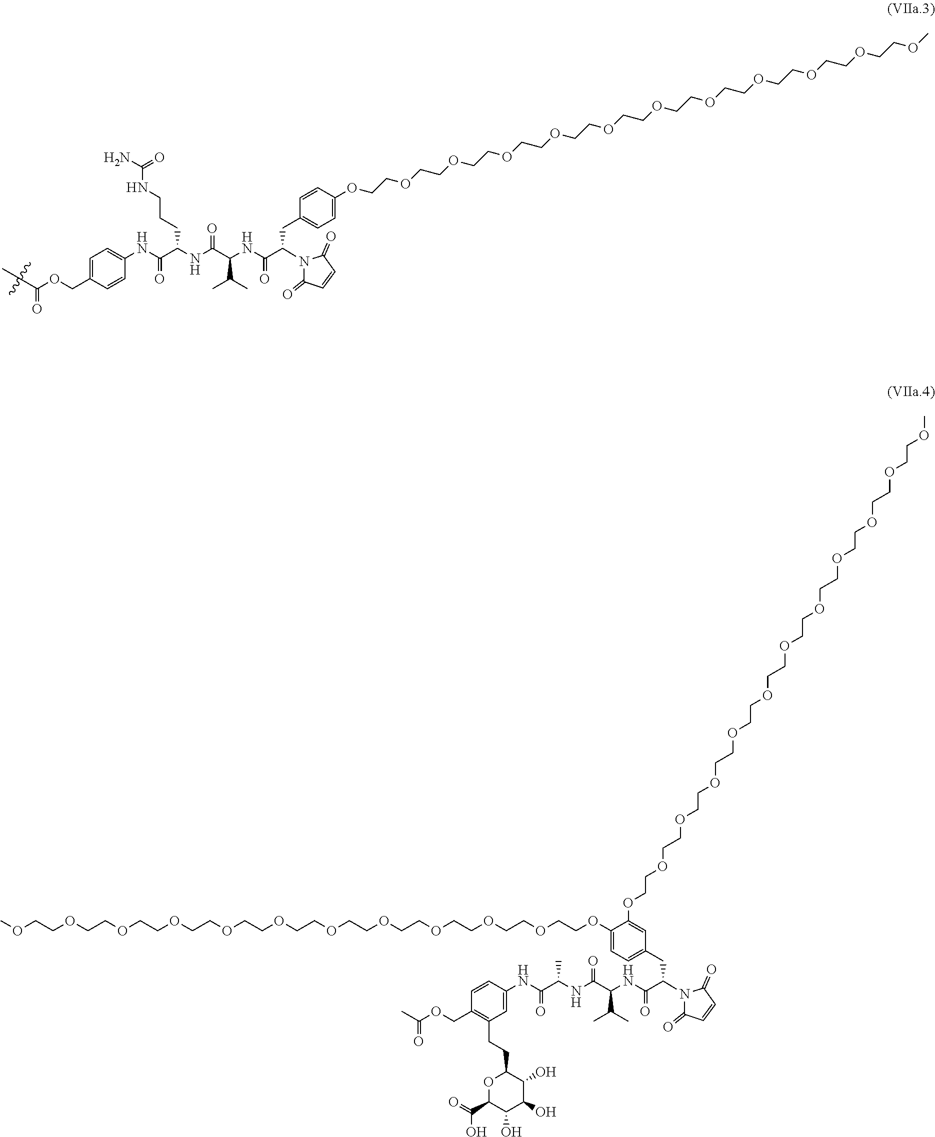

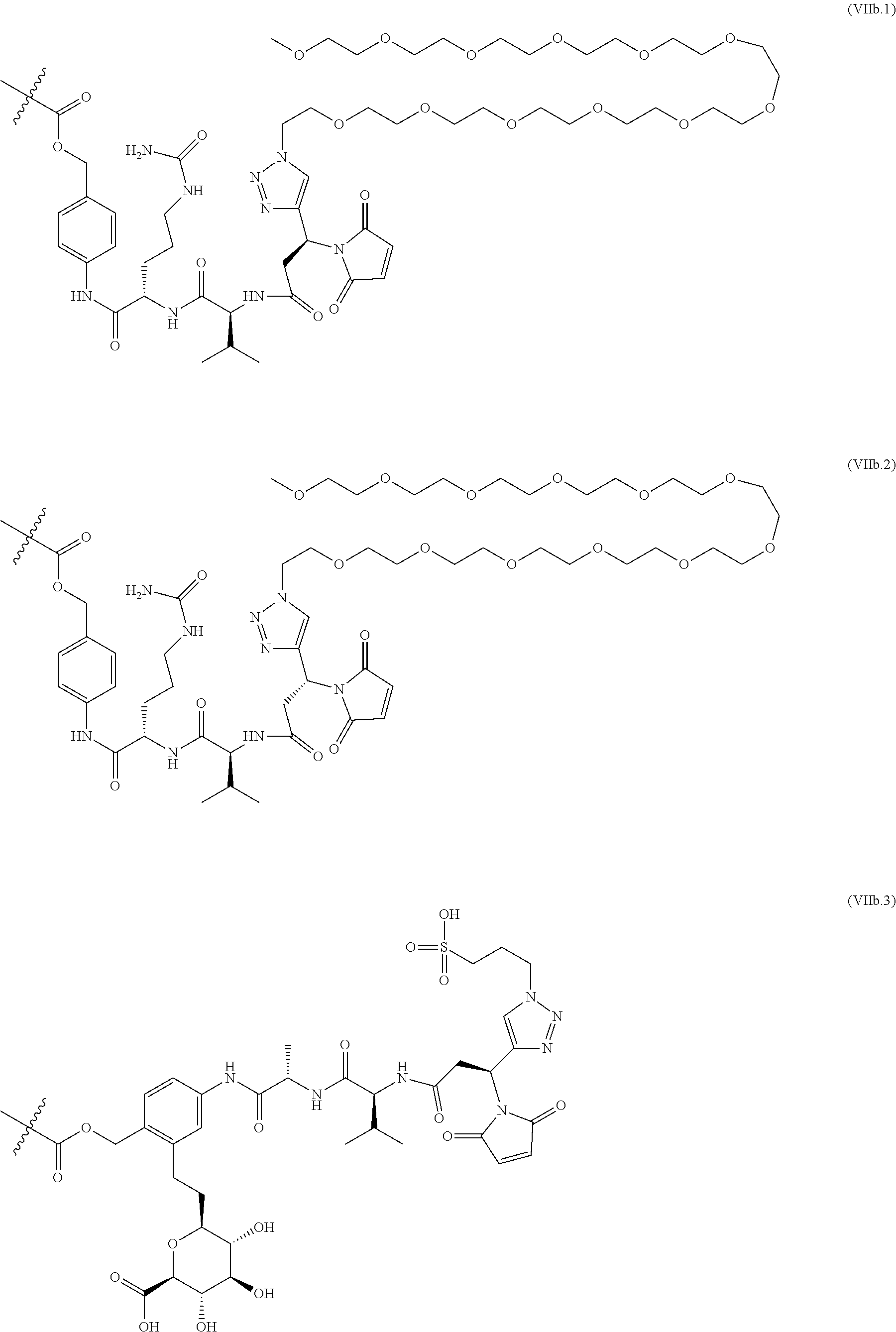 [SMILES: *C(=O)OCc1ccc(NC(=O)[C@H](CCCNC(N)=O)NC(=O)[C@@H](NC(=O)C[C@@H](c2cn(CCOCCOCCOCCOCCOCCOCCOCCOCCOCCOCCOCCOC)nn2)N2C(=O)C=CC2=O)C(C)C)cc1 ; *C(=O)OCc1ccc(NC(=O)[C@H](CCCNC(N)=O)NC(=O)[C@@H](NC(=O)C[C@H](c2cn(CCOCCOCCOCCOCCOCCOCCOCCOCCOCCOCCOCCOC)nn2)N2C(=O)C=CC2=O)C(C)C)cc1 ; *C(=O)OCc1ccc(NC(=O)[C@H](C)NC(=O)[C@@H](NC(=O)C[C@@H](c2cn(CCCS(=O)(=O)O)nn2)N2C(=O)C=CC2=O)C(C)C)cc1CC[C@@H]1O[C@H](C(=O)O)[C@@H](O)[C@H](O)[C@H]1O]

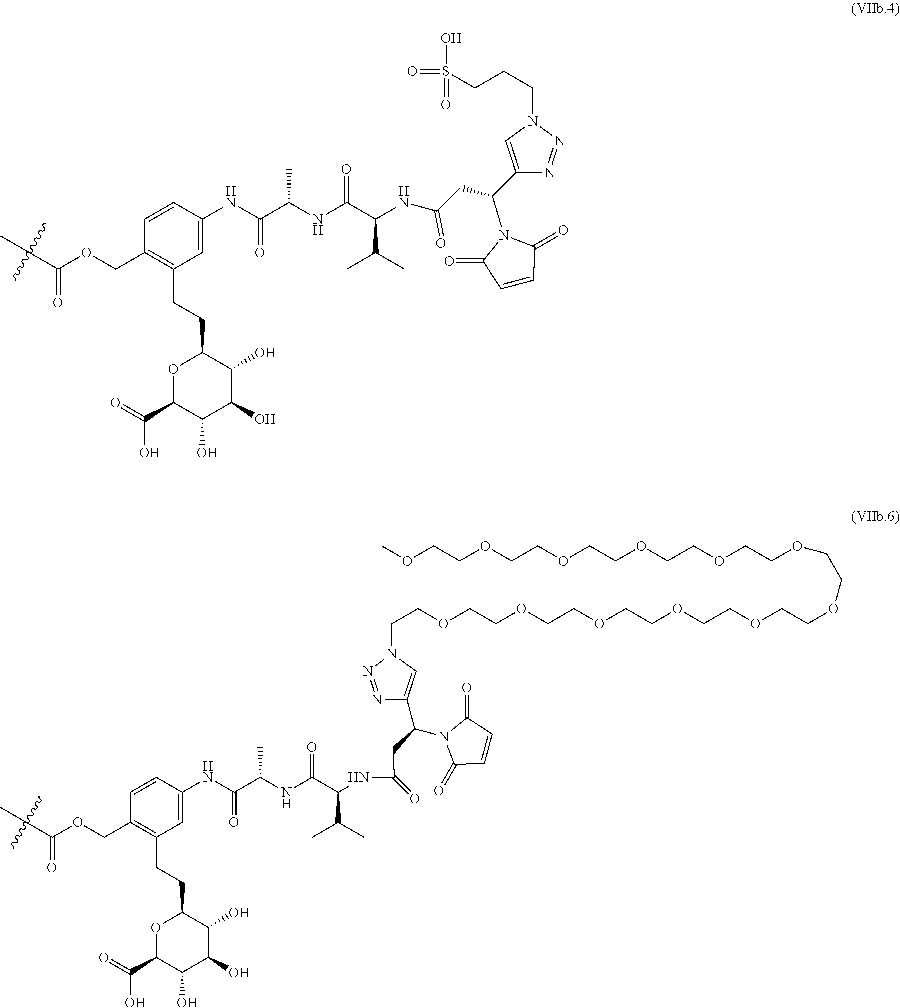 [SMILES: *C(=O)OCc1ccc(NC(=O)[C@H](C)NC(=O)[C@@H](NC(=O)C[C@H](c2cn(CCCS(=O)(=O)O)nn2)N2C(=O)C=CC2=O)C(C)C)cc1CC[C@@H]1O[C@H](C(=O)O)[C@@H](O)[C@H](O)[C@H]1O ; *C(=O)OCc1ccc(NC(=O)[C@H](C)NC(=O)[C@@H](NC(=O)C[C@@H](c2cn(CCOCCOCCOCCOCCOCCOCCOCCOCCOCCOCCOCCOC)nn2)N2C(=O)C=CC2=O)C(C)C)cc1CC[C@@H]1O[C@H](C(=O)O)[C@@H](O)[C@H](O)[C@H]1O]

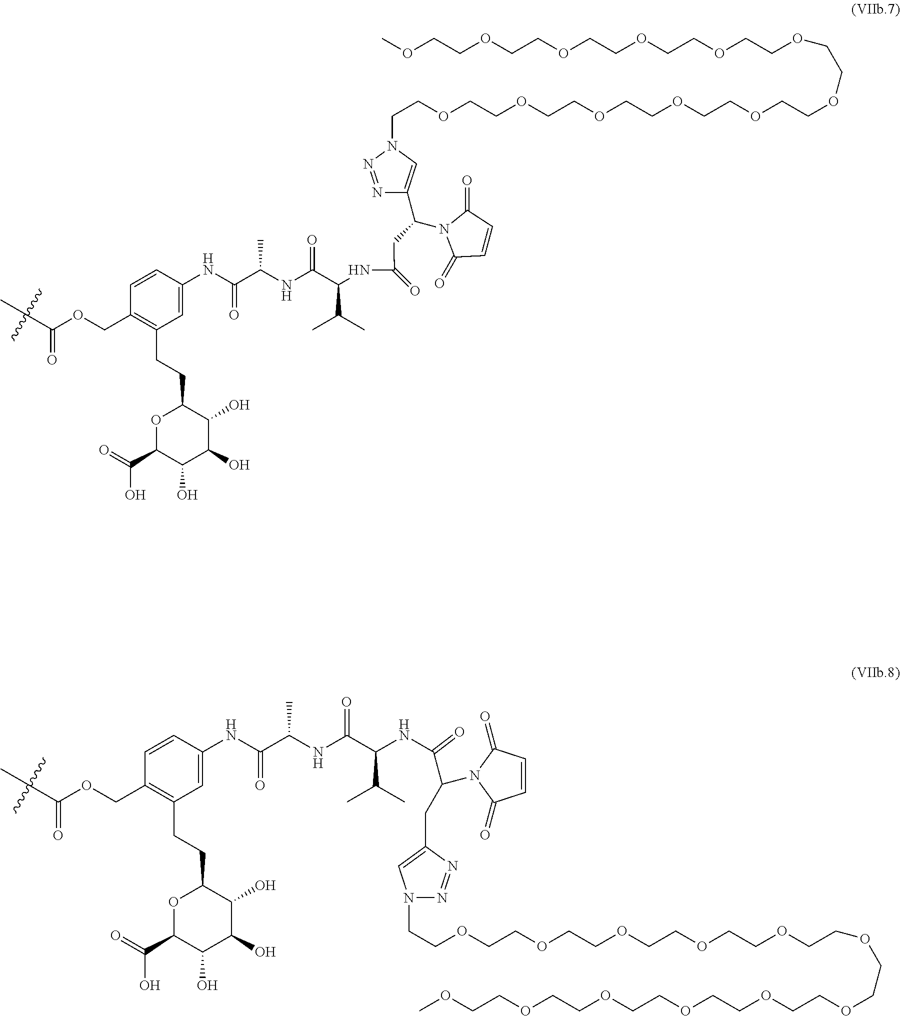 [SMILES: *C(=O)OCc1ccc(NC(=O)[C@H](C)NC(=O)[C@@H](NC(=O)C[C@H](c2cn(CCOCCOCCOCCOCCOCCOCCOCCOCCOCCOCCOCCOC)nn2)N2C(=O)C=CC2=O)C(C)C)cc1CC[C@@H]1O[C@H](C(=O)O)[C@@H](O)[C@H](O)[C@H]1O ; *C(=O)OCc1ccc(NC(=O)[C@H](C)NC(=O)[C@@H](NC(=O)C(Cc2cn(CCOCCOCCOCCOCCOCCOCCOCCOCCOCCOCCOCCOC)nn2)N2C(=O)C=CC2=O)C(C)C)cc1CC[C@@H]1O[C@H](C(=O)O)[C@@H](O)[C@H](O)[C@H]1O]

Exemplary embodiments of linkers according to structural formula (VIIc) that may be included in the ADCs described herein include the linkers illustrated below (as illustrated, the linkers include a group suitable for covalently linking the linker to an antibody):

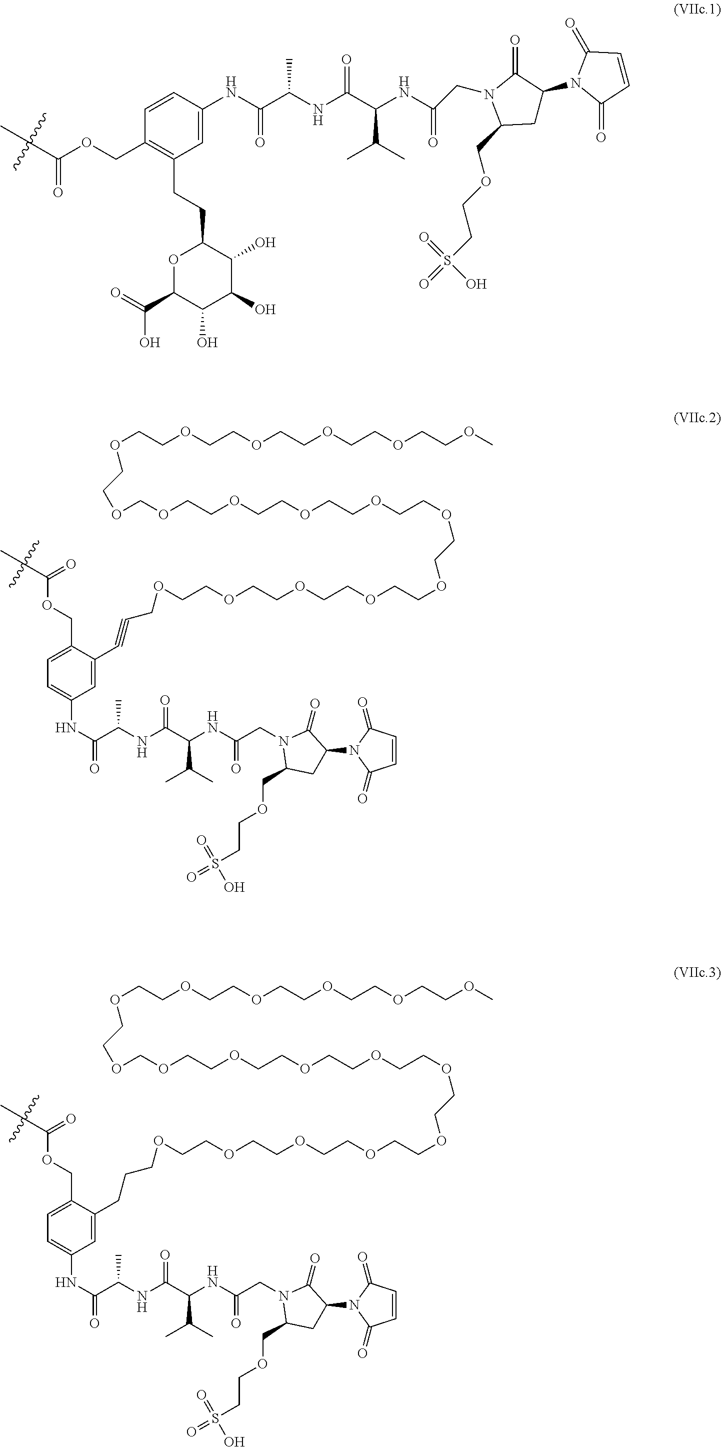 [SMILES: *C(=O)OCc1ccc(NC(=O)[C@H](C)NC(=O)[C@@H](NC(=O)CN2C(=O)[C@@H](N3C(=O)C=CC3=O)C[C@H]2COCCS(=O)(=O)O)C(C)C)cc1CC[C@@H]1O[C@H](C(=O)O)[C@@H](O)[C@H](O)[C@H]1O ; *C(=O)OCc1ccc(NC(=O)[C@H](C)NC(=O)[C@@H](NC(=O)CN2C(=O)[C@@H](N3C(=O)C=CC3=O)C[C@H]2COCCS(=O)(=O)O)C(C)C)cc1C#CCOCCOCCOCCOCCOCCOCCOCCOCCOCCOCOCCOCCOCCOCCOCCOCCOC ; *C(=O)OCc1ccc(NC(=O)[C@H](C)NC(=O)[C@@H](NC(=O)CN2C(=O)[C@@H](N3C(=O)C=CC3=O)C[C@H]2COCCS(=O)(=O)O)C(C)C)cc1CCCOCCOCCOCCOCCOCCOCCOCCOCCOCCOCOCCOCCOCCOCCOCCOCCOC]

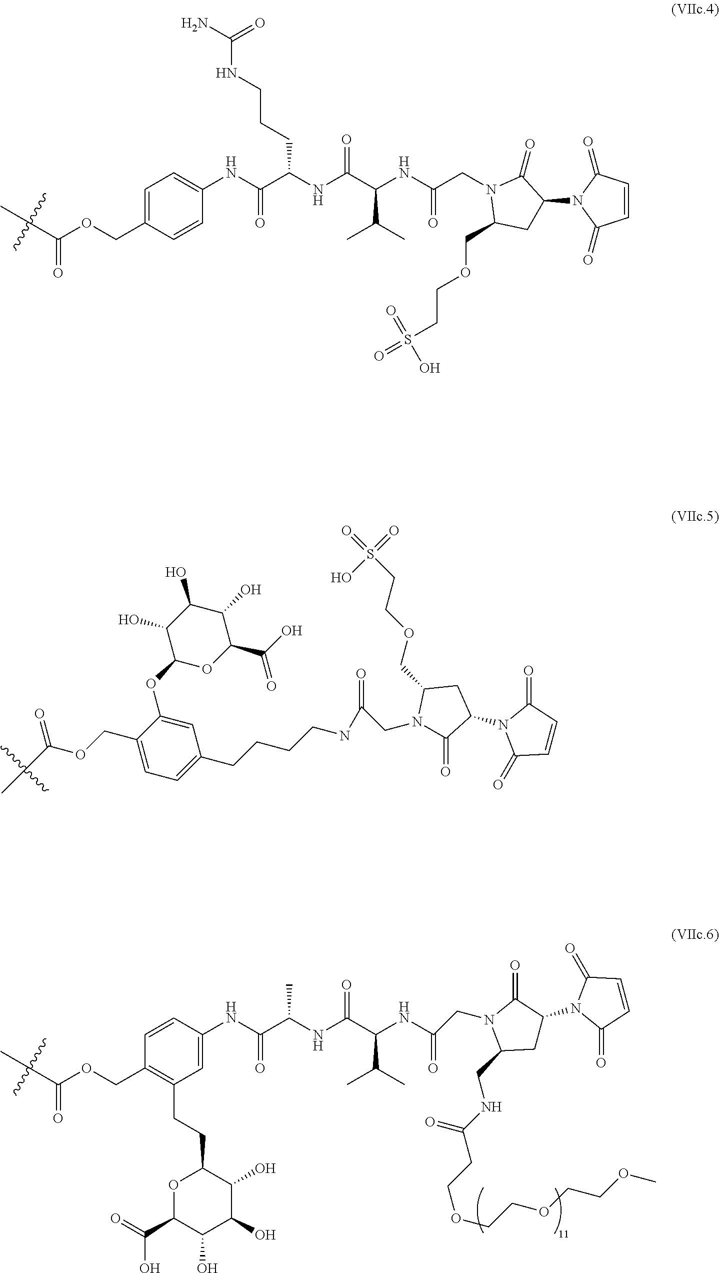

In certain embodiments, L is selected from the group consisting of IVa.1-IVa.8, IVb.1-IVb.19, IVc.1-IVc.7, IVd.1-IVd.4, Va.1-Va.12, Vb.1-Vb.10, Vc.1-Vc.11. Vd.1-Vd.6, Ve.1-Ve.2, VIa.1, VIc.1-VIc.2, VId.1-VId.4, VIIa.1-VIIa.4, VIIb.1-VIIb.8. VIIc.1-VIIc.6 in either the closed or open form.

In certain embodiments, L is selected from the group consisting of IVb.2, IVc.5, IVc.6, IVc.7, IVd.4. Vb.9, Vc.11, VIIa.1, VIIa.3, VIIc.1. VIIc.4, and VIIc.5, wherein the maleimide of each linker has reacted with the antibody Ab, forming a covalent attachment as either a succinimide (closed form) or succinamide (open form).

In certain embodiments, linker L is selected from the group consisting of IVb.2, IVc.5. IVc.6, IVd.4, Vc.11, VIIa.1, VIIa.3, VIIe.1, VIIc.4, VIIc.5, wherein the maleimide of each linker has reacted with the antibody Ab, forming a covalent attachment as either a succinimide (closed form) or succinamide (open form).

In certain embodiments, linker L is selected from the group consisting of IVb.2, Vc.11, VIIa.3, IVc.6, and VIIe.1, wherein [symbol] is the attachment point to drug D and @ is the attachment point to the LK, wherein when the linker is in the open form as shown below, @ can be either at the α-position or β-position of the carboxylic acid next to it:

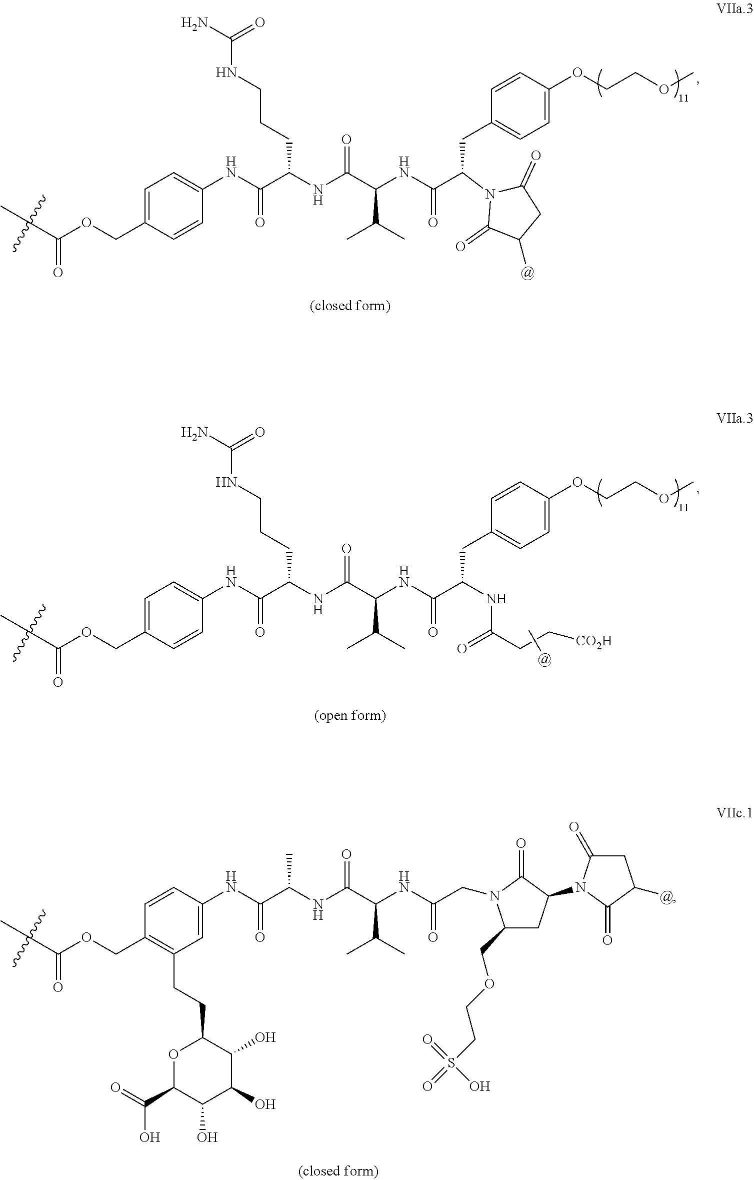

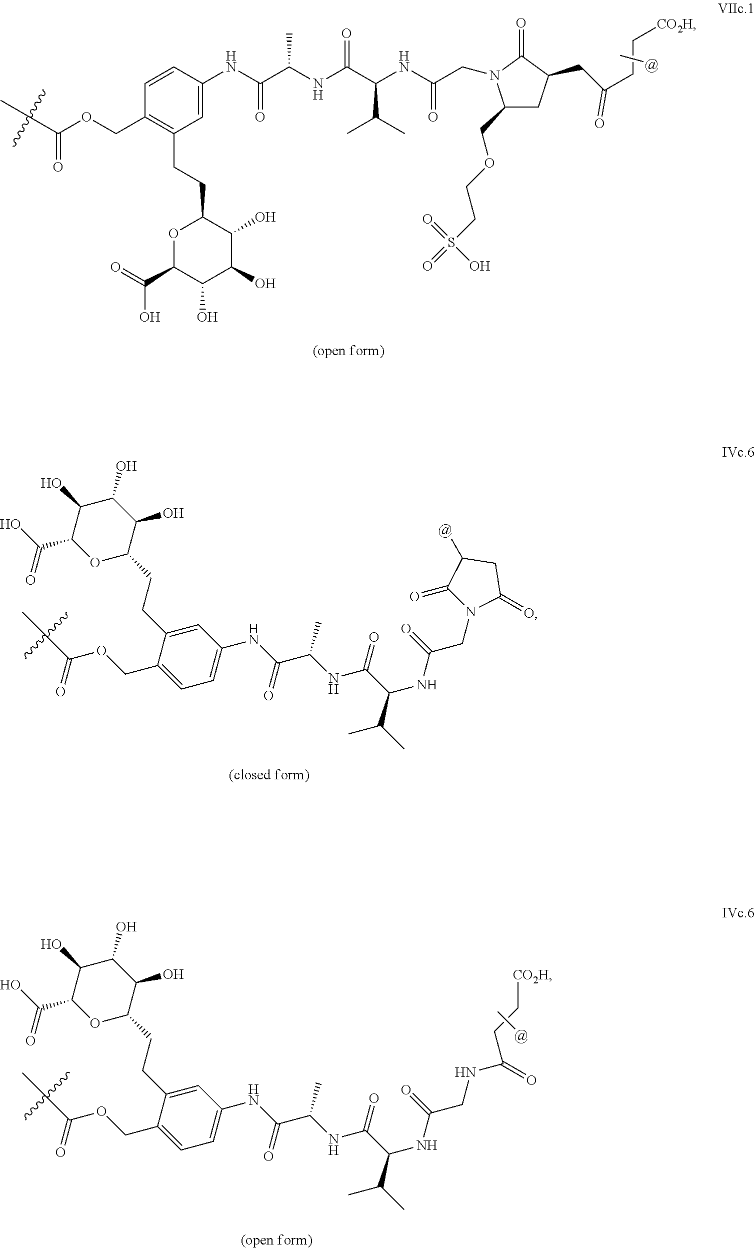

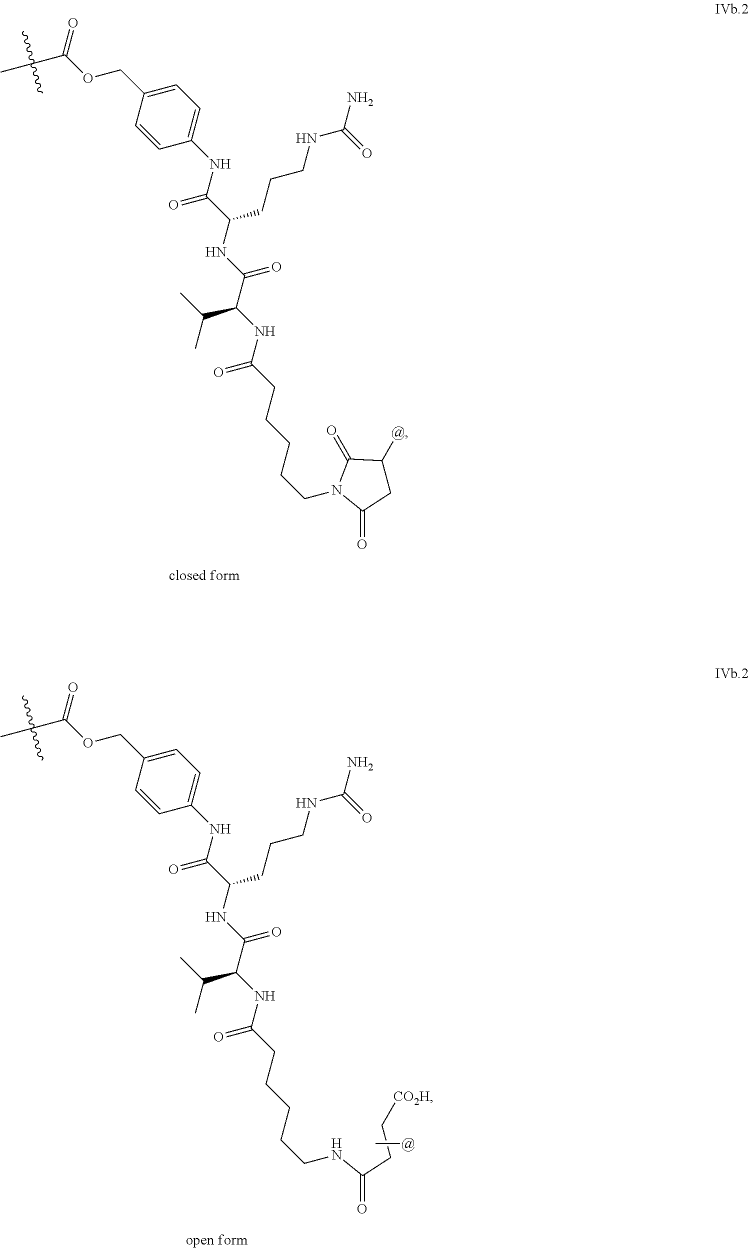

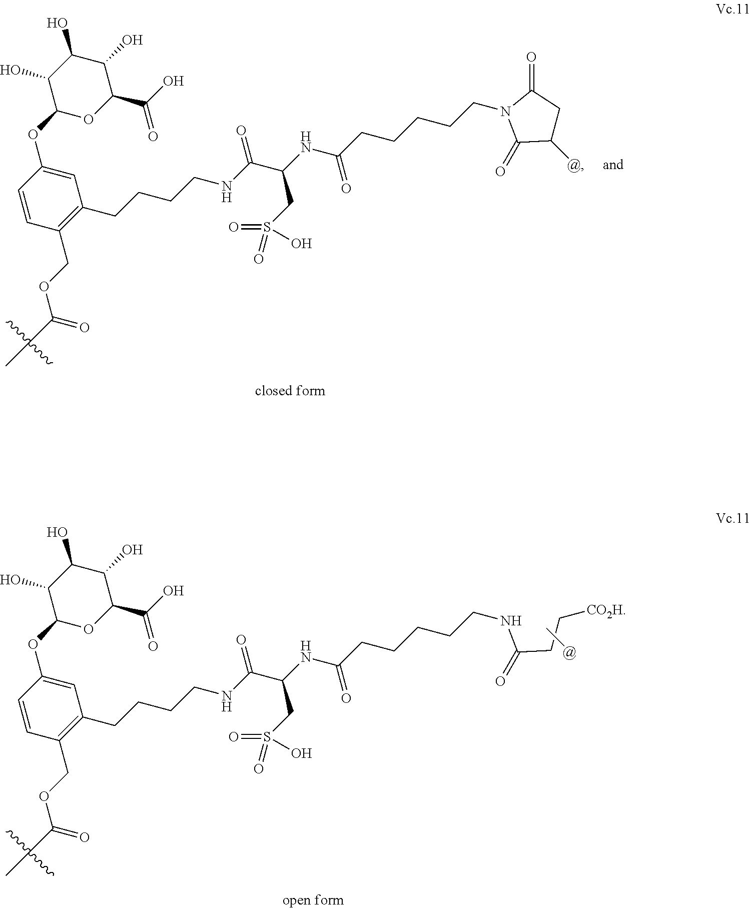

### Bcl-xL Linker Selection Considerations

As is known by skilled artisans, the linker selected for a particular ADC may be influenced by a variety of factors, including but not limited to, the site of attachment to the antibody (e.g., lys, cys or other amino acid residues), structural constraints of the drug pharmacophore and the lipophilicity of the drug. The specific linker selected for an ADC should seek to balance these different factors for the specific antibody/drug combination. For a review of the factors that are influenced by choice of linkers in ADCs, see Nolting. Chapter 5 “Linker Technology in Antibody-Drug Conjugates.” In: Antibody-Drug Conjugates: Methods in Molecular Biology, vol. 1045, pp. 71-100, Laurent Ducry (Ed.). Springer Science & Business Medica, LLC, 2013.

For example, ADCs have been observed to effect killing of bystander antigen-negative cells present in the vicinity of the antigen-positive tumor cells. The mechanism of bystander cell killing by ADCs has indicated that metabolic products formed during intracellular processing of the ADCs may play a role. Neutral cytotoxic metabolites generated by metabolism of the ADCs in antigen-positive cells appear to play a role in bystander cell killing while charged metabolites may be prevented from diffusing across the membrane into the medium and therefore cannot affect bystander killing. In certain embodiments, the linker is selected to attenuate the by stander killing effect caused by cellular metabolites of the ADC. In certain embodiments, the linker is selected to increase the bystander killing effect.

The properties of the linker may also impact aggregation of the ADC under conditions of use and/or storage. Typically. ADCs reported in the literature contain no more than 3-4 drug molecules per antibody molecule (see, e.g., Chari, 2008, Acc Chem Res 41:98-107). Attempts to obtain higher drug-to-antibody ratios (“DAR”) often failed, particularly if both the drug and the linker were hydrophobic, due to aggregation of the ADC (King et al., 2002, J Med Chem 45:4336-4343; Hollander et al., 2008. Bioconjugate Chem 19:358-361; Burke et al., 2009 Bioconjugate Chem 20:1242-1250). In many instances, DARs higher than 3-4 could be beneficial as a means of increasing potency. In instances where the Bcl-xL inhibitor is hydrophobic in nature, it may be desirable to select linkers that are relatively hydrophilic as a means of reducing ADC aggregation, especially in instances where DARS greater than 3-4 are desired. Thus, in certain embodiments, the linker incorporates chemical moieties that reduce aggregation of the ADCs during storage and/or use. A linker may incorporate polar or hydrophilic groups such as charged groups or groups that become charged under physiological pH to reduce the aggregation of the ADCs. For example, a linker may incorporate charged groups such as salts or groups that deprotonate, e.g., carboxylates, or protonate, e.g., amines, at physiological pH.

Exemplary polyvalent linkers that have been reported to yield DARs as high as 20 that may be used to link numerous Bcl-xL inhibitors to an antibody are described in U.S. Pat. No. 8,399,512; U.S. Published Application No. 2010/0152725; U.S. Pat. Nos. 8,524,214; 8,349,308; U.S. Published Application No. 2013/189218; U.S. Published Application No. 2014/017265; WO 2014/093379; WO 2014/093394; WO 2014/093640, the content of which are incorporated herein by reference in their entireties.

In particular embodiments, the aggregation of the ADCs during storage or use is less than about 40% as determined by size-exclusion chromatography (SEC). In particular embodiments, the aggregation of the ADCs during storage or use is less than 35%, such as less than about 30%, such as less than about 25%, such as less than about 20%, such as less than about 15%, such as less than about 10%, such as less than about 5%, such as less than about 4%, or even less, as determined by size-exclusion chromatography (SEC).

### IIIA.3. Bcl-xL ADC Synthons

Antibody-Drag Conjugate synthons are synthetic intermediates used to form ADCs. The synthons are generally compounds according to structural formula (III):

D-L-R^{x} (III)

or salts thereof, wherein D is a Bcl-xL inhibitor as previously described, L is a linker as previously described, and R^{x} is a reactive group suitable for linking the synthon to an antibody. In specific embodiments, the ADC synthons are compounds according to structural formulae (IIIa) and (IIIb), or salts thereof, where the various substituents are as previously defined for structural formulae (IIa) and (IIb), respectively, and L and R^{x} are as defined for structural formula (III):

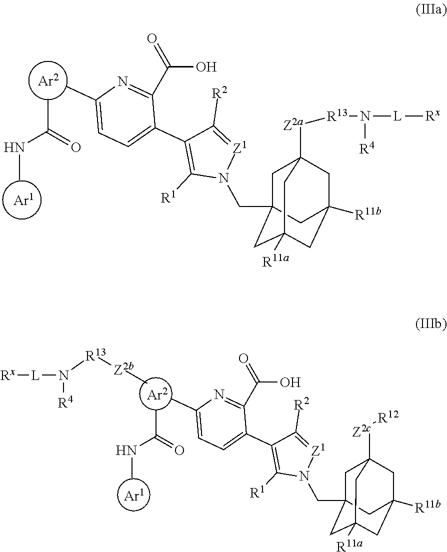

To synthesize an ADC, an intermediate synthon according to structural formula (III), or a salt thereof, is contacted with an antibody of interest under conditions in which functional group R^{x} reacts with a “complementary” functional group on the antibody, F^{x}, to form a covalent linkage.

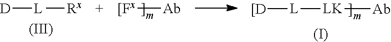

The identities of groups R^{x} and F^{x} will depend upon the chemistry used to link the synthon to the antibody. Generally, the chemistry used should not alter the integrity of the antibody, for example its ability to bind its target. Preferably, the binding properties of the conjugated antibody will closely resemble those of the unconjugated antibody. A variety of chemistries and techniques for conjugating molecules to biological molecules such as antibodies are known in the art and in particular to antibodies, are well-known. See, e.g., Amon et al., “Monoclonal Antibodies For Immunotargeting Of Drugs In Cancer Therapy,” in: Monoclonal Antibodies And Cancer Therapy, Reisfeld et al., Eds., Alan R. Liss, Inc., 1985; Hellstrom et al., “Antibodies For Drug Delivery.” in: Controlled Drug Delivery, Robinson et al., Eds., Marcel Dekker, Inc., 2nd Ed. 1987; Thorpe, “Antibody Carriers Of Cytotoxic Agents In Cancer Therapy: A Review,” in: Monoclonal Antibodies '84: Biological And Clinical Applications, Pinchera et al., Eds., 1985; “Analysis. Results, and Future Prospective of the Therapeutic Use of Radiolabeled Antibody In Cancer Therapy,” in: Monoclonal Antibodies For Cancer Detection And Therapy, Baldwin et al., Eds., Academic Press, 1985; Thorpe et al., 1982, Immunol. Rev. 62:119-58; PCT publication WO 89/12624. Any of these chemistries may be used to link the synthons to an antibody.

In one embodiment, R^{x} comprises a functional group capable of linking the synthon to an amino group on an antibody. In another embodiment, R^{x} comprises an NHS-ester or an isothiocyanate. In another embodiment, R^{x} comprises a functional group capable of linking the synthon to a sulfhydryl group on an antibody. In another embodiment, R^{x} comprises a haloacetyl or a maleimide. In another embodiment, L is selected from IVa or IVb and salts thereof; and R^{x} comprises a functional group selected from the group consisting of NHS-ester, isothiocyanate, haloacetyl and maleimide.

Typically, the synthons are linked to the side chains of amino acid residues of the antibody, including, for example, the primary amino group of accessible lysine residues or the sulfhydryl group of accessible cysteine residues. Free sulfhydryl groups may be obtained by reducing interchain disulfide bonds.

In one embodiment LK is a linkage formed with an amino group on the anti-hCD98 antibody Ab. In another embodiment, LK is an amide or a thiourea. In another embodiment. LK is a linkage formed with a sulfhydryl group on the anti-hCD98 antibody Ab. In another embodiment, LK is a thioether.

In one embodiment, LK is selected from the group consisting of amide, thiourea and thioether; and m is an integer ranging from 1 to 8.

A number of functional groups R^{x} and chemistries useful for linking synthons to accessible lysine residues are known, and include by way of example and not limitation NHS-esters and isothiocyanates.

A number of functional groups R^{x} and chemistries useful for linking synthons to accessible free sulfhydryl groups of cysteine residues are known, and include by way of example and not limitation haloacetyls and maleimides.

However, conjugation chemistries are not limited to available side chain groups. Side chains such as amines may be converted to other useful groups, such as hydroxyls, by linking an appropriate small molecule to the amine. This strategy can be used to increase the number of available linking sites on the antibody by conjugating multifunctional small molecules to side chains of accessible amino acid residues of the antibody. Functional groups R^{x} suitable for covalently linking the synthons to these “converted” functional groups are then included in the synthons.

The antibody may also be engineered to include amino acid residues for conjugation. An approach for engineering antibodies to include non-genetically encoded amino acid residues useful for conjugating drugs in the context of ADCs is described in Axup et al., 2003, Proc Natl Acad Sci 109:16101-16106 and Tian et al., 2014. Proc Natl Acad Sci 111:1776-1771, as are chemistries and functional group useful for linking synthons to the non-encoded amino acids.

Exemplary synthons useful for making ADCs described herein include, but are not limited to, the following synthons listed below in Table 5.

TABLE 5

| Appln | | |
|---|---|---|
| Ex. No. | Synthon | Synthon Structure |
| 2.1 | BS | 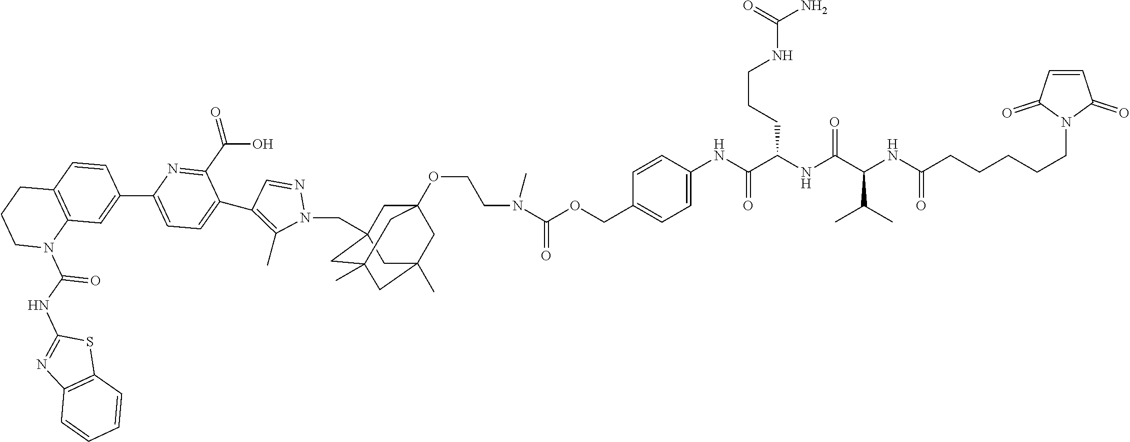 [SMILES: Cc1c(-c2ccc(-c3ccc4c(c3)N(C(=O)Nc3nc5ccccc5s3)CCC4)nc2C(=O)O)cnn1CC12CC3(C)CC(C)(C1)CC(OCCN(C)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.2 | DK | 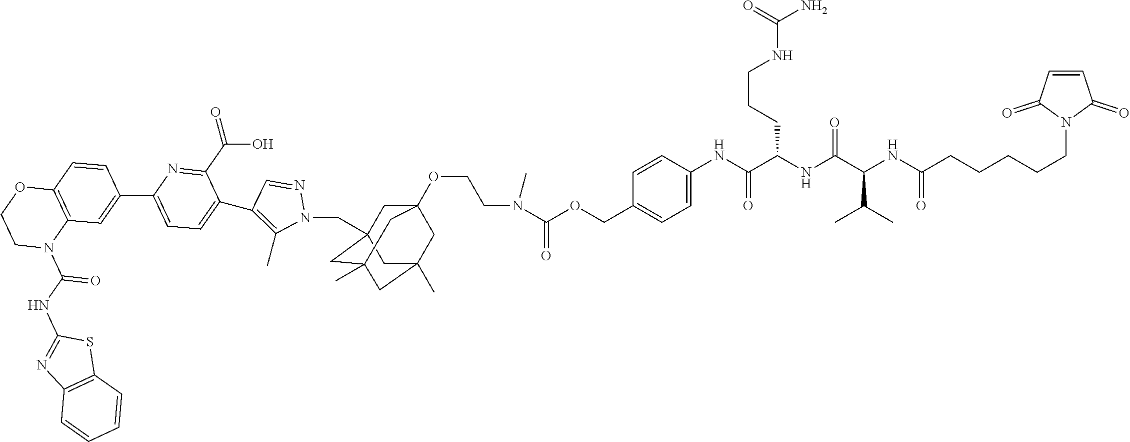 [SMILES: Cc1c(-c2ccc(-c3ccc4c(c3)N(C(=O)Nc3nc5ccccc5s3)CCO4)nc2C(=O)O)cnn1CC12CC3(C)CC(C)(C1)CC(OCCN(C)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.3 | DQ | 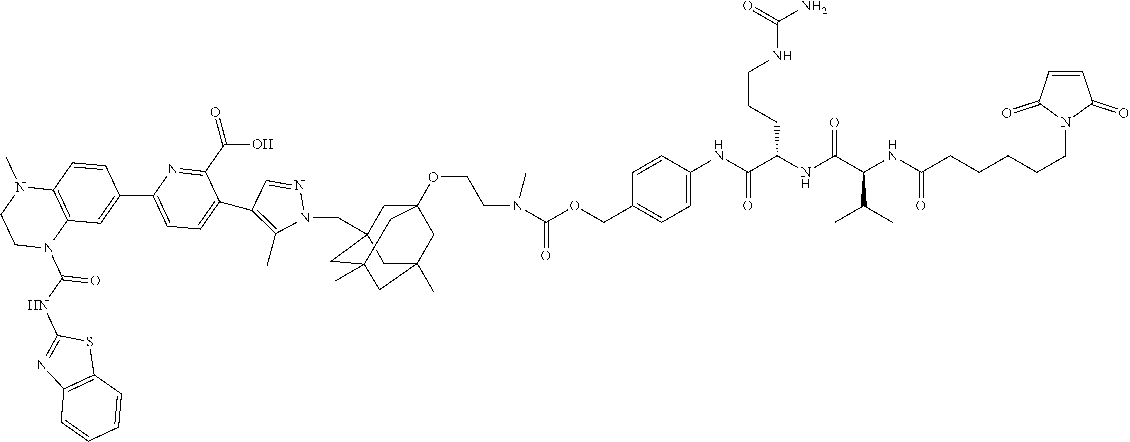 [SMILES: Cc1c(-c2ccc(-c3ccc4c(c3)N(C(=O)Nc3nc5ccccc5s3)CCN4C)nc2C(=O)O)cnn1CC12CC3(C)CC(C)(C1)CC(OCCN(C)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.4 | DJ | 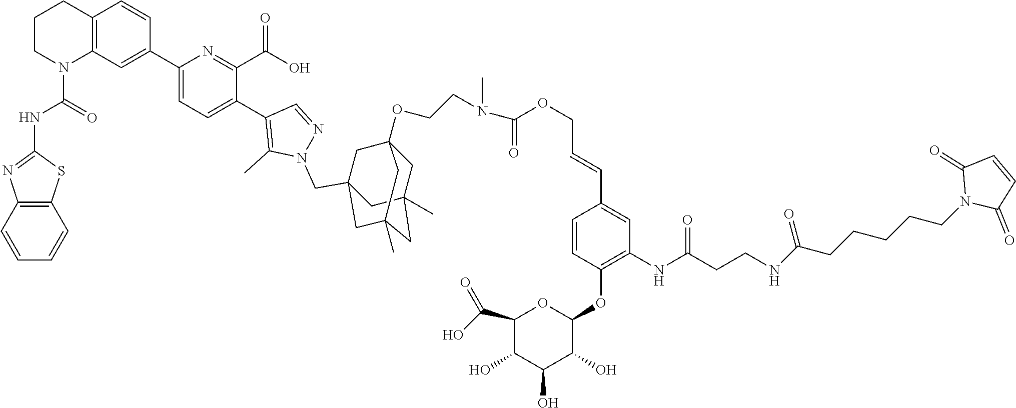 [SMILES: Cc1c(-c2ccc(-c3ccc4c(c3)N(C(=O)Nc3nc5ccccc5s3)CCC4)nc2C(=O)O)cnn1CC12CC3(C)CC(C)(C1)CC(OCCN(C)C(=O)OC/C=C/c1ccc(O[C@@H]4O[C@H](C(=O)O)[C@@H](O)[C@H](O)[C@H]4O)c(NC(=O)CCNC(=O)CCCCCN4C(=O)C=CC4=O)c1)(C3)C2] |
| 2.5 | DO | 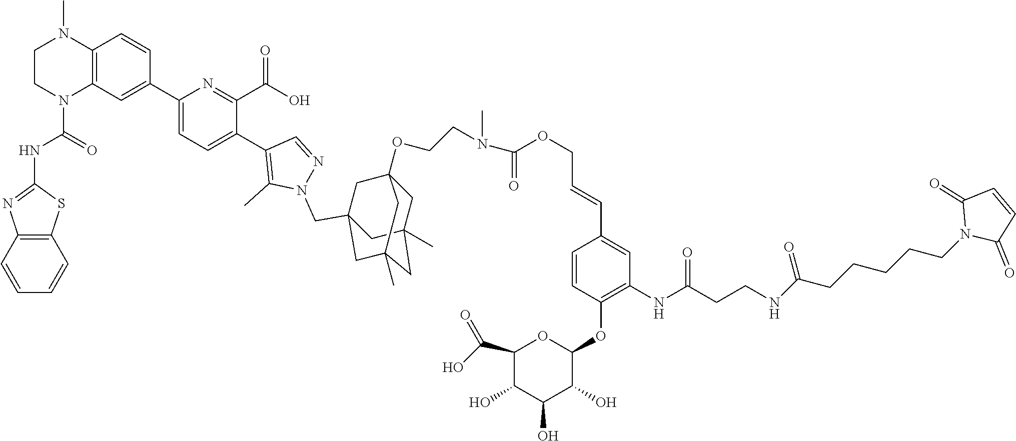 [SMILES: Cc1c(-c2ccc(-c3ccc4c(c3)N(C(=O)Nc3nc5ccccc5s3)CCN4C)nc2C(=O)O)cnn1CC12CC3(C)CC(C)(C1)CC(OCCN(C)C(=O)OC/C=C/c1ccc(O[C@@H]4O[C@H](C(=O)O)[C@@H](O)[C@H](O)[C@H]4O)c(NC(=O)CCNC(=O)CCCCCN4C(=O)C=CC4=O)c1)(C3)C2] |
| 2.6 | DP | 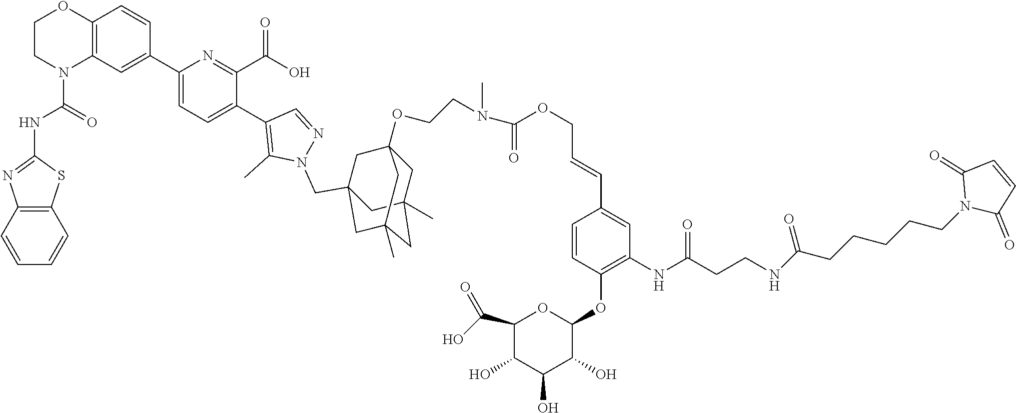 [SMILES: Cc1c(-c2ccc(-c3ccc4c(c3)N(C(=O)Nc3nc5ccccc5s3)CCO4)nc2C(=O)O)cnn1CC12CC3(C)CC(C)(C1)CC(OCCN(C)C(=O)OC/C=C/c1ccc(O[C@@H]4O[C@H](C(=O)O)[C@@H](O)[C@H](O)[C@H]4O)c(NC(=O)CCNC(=O)CCCCCN4C(=O)C=CC4=O)c1)(C3)C2] |
| 2.7 | HO | 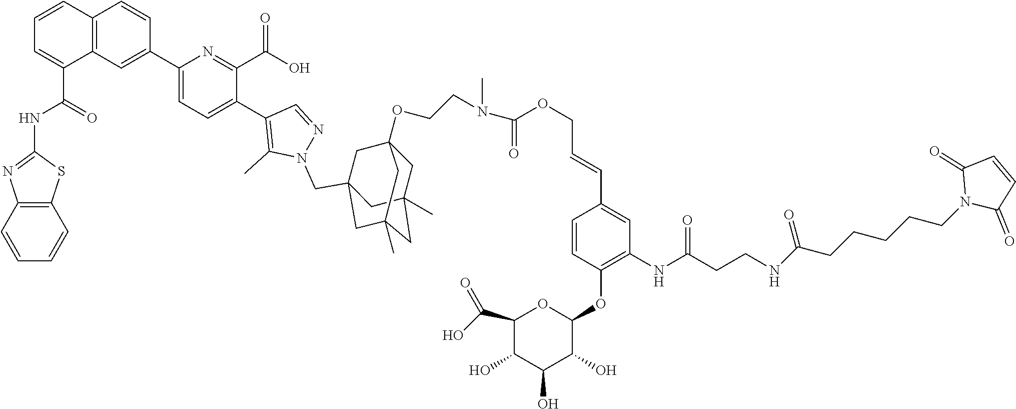 [SMILES: Cc1c(-c2ccc(-c3ccc4cccc(C(=O)Nc5nc6ccccc6s5)c4c3)nc2C(=O)O)cnn1CC12CC3(C)CC(C)(C1)CC(OCCN(C)C(=O)OC/C=C/c1ccc(O[C@@H]4O[C@H](C(=O)O)[C@@H](O)[C@H](O)[C@H]4O)c(NC(=O)CCNC(=O)CCCCCN4C(=O)C=CC4=O)c1)(C3)C2] |
| 2.8 | IT | 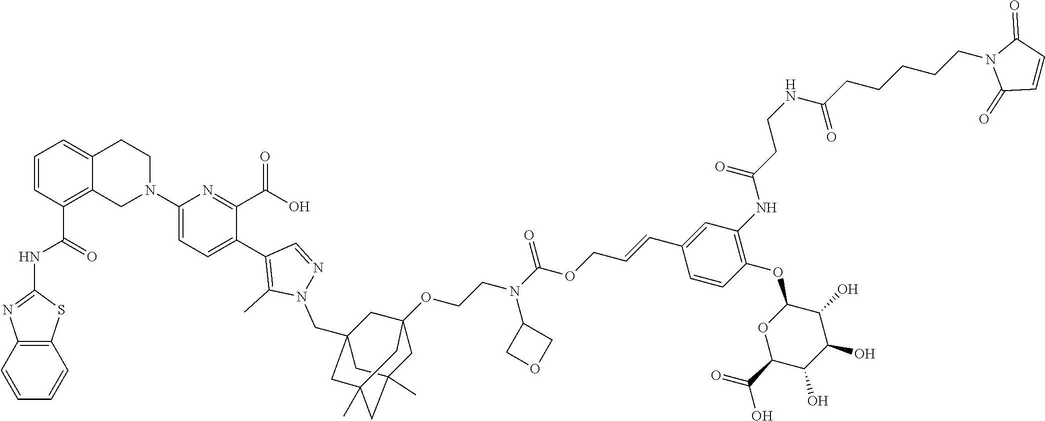 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(C(=O)OC/C=C/c1ccc(O[C@@H]4O[C@H](C(=O)O)[C@@H](O)[C@H](O)[C@H]4O)c(NC(=O)CCNC(=O)CCCCCN4C(=O)C=CC4=O)c1)C1COC1)(C3)C2] |
| 2.9 | KA | 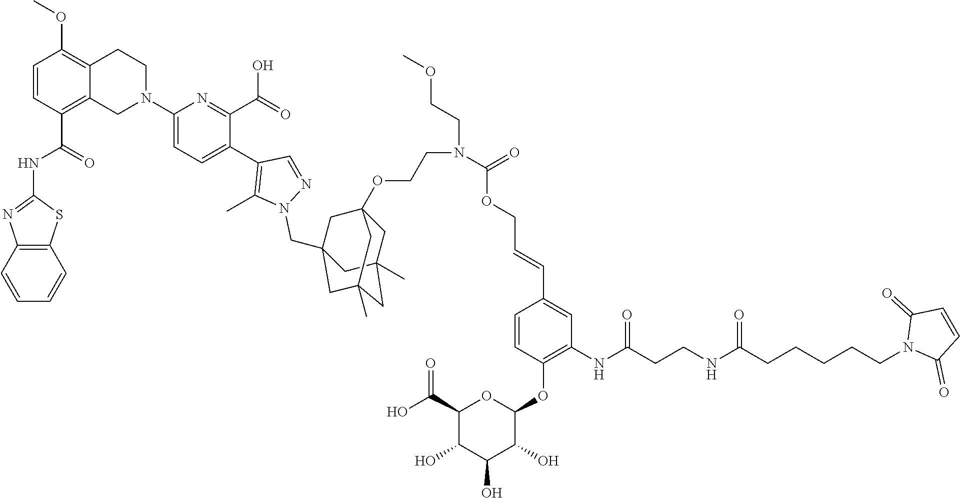 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OC/C=C/c1ccc(O[C@@H]2O[C@H](C(=O)O)[C@@H](O)[C@H](O)[C@H]2O)c(NC(=O)CCNC(=O)CCCCCN2C(=O)C=CC2=O)c1] |
| 2.10 | KB | 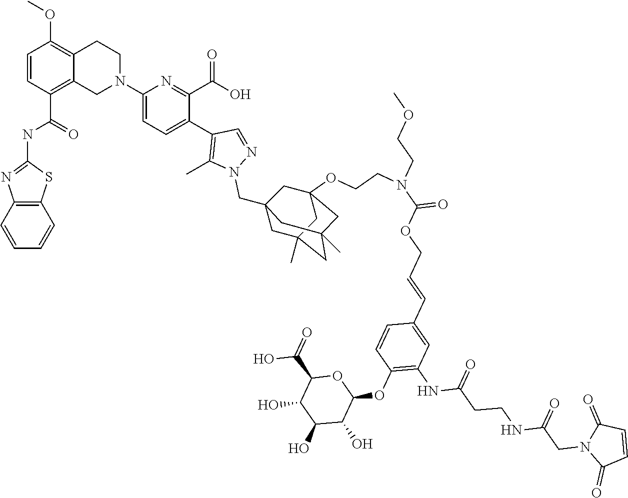 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OC/C=C/c1ccc(O[C@@H]2O[C@H](C(=O)O)[C@@H](O)[C@H](O)[C@H]2O)c(NC(=O)CCNC(=O)CN2C(=O)C=CC2=O)c1] |
| 2.11 | KT | 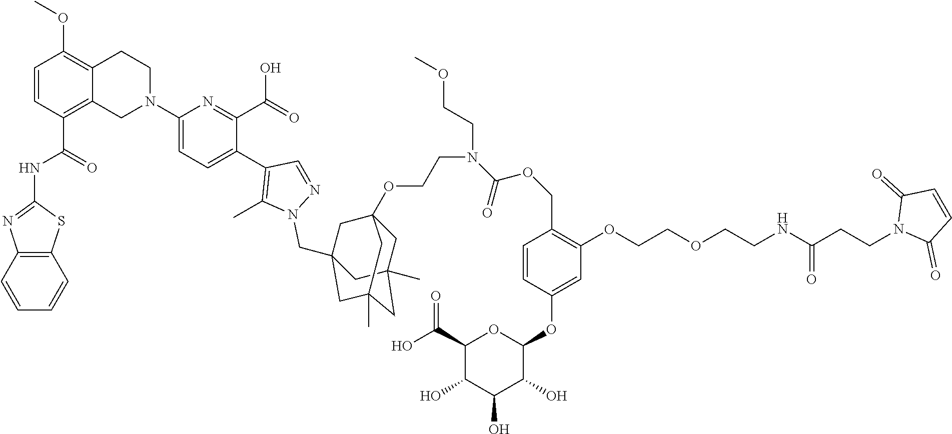 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OCc1ccc(O[C@@H]2O[C@H](C(=O)O)[C@@H](O)[C@H](O)[C@H]2O)cc1OCCOCCNC(=O)CCN1C(=O)C=CC1=O] |
| 2.12 | KU | 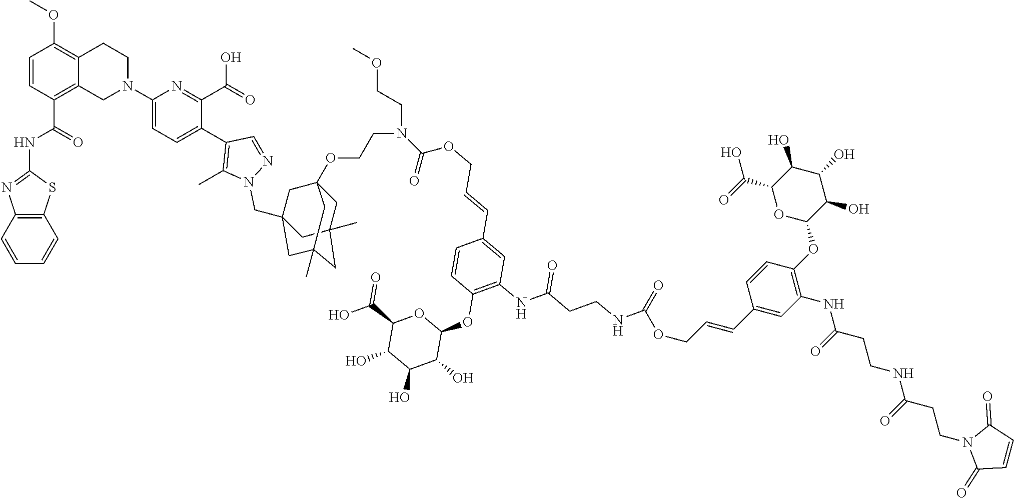 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OC/C=C/c1ccc(O[C@@H]2O[C@H](C(=O)O)[C@@H](O)[C@H](O)[C@H]2O)c(NC(=O)CCNC(=O)OC/C=C/c2ccc(O[C@@H]3O[C@H](C(=O)O)[C@@H](O)[C@H](O)[C@H]3O)c(NC(=O)CCNC(=O)CCN3C(=O)C=CC3=O)c2)c1] |
| 2.13 | KV | 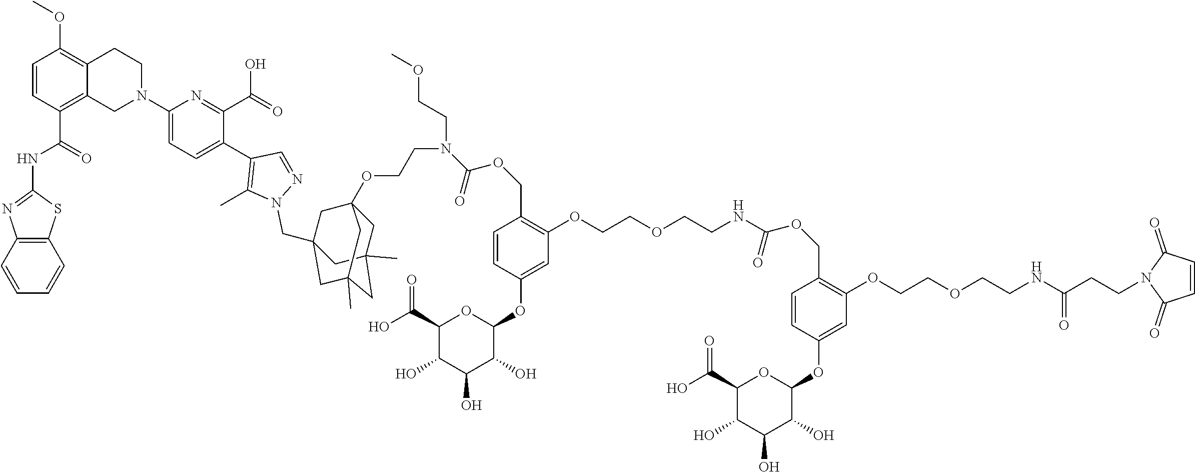 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OCc1ccc(O[C@@H]2O[C@H](C(=O)O)[C@@H](O)[C@H](O)[C@H]2O)cc1OCCOCCNC(=O)OCc1ccc(O[C@@H]2O[C@H](C(=O)O)[C@@H](O)[C@H](O)[C@H]2O)cc1OCCOCCNC(=O)CCN1C(=O)C=CC1=O] |
| 2.14 | KW | 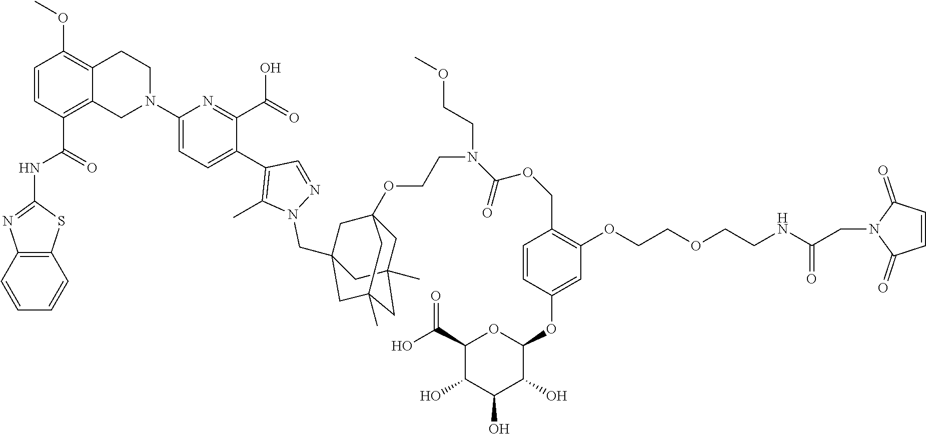 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OCc1ccc(O[C@@H]2O[C@H](C(=O)O)[C@@H](O)[C@H](O)[C@H]2O)cc1OCCOCCNC(=O)CN1C(=O)C=CC1=O] |
| 2.15 | DC | 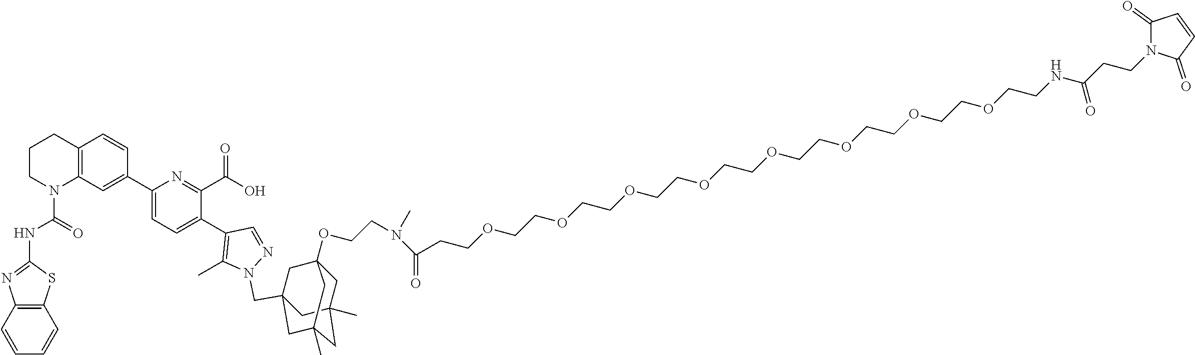 [SMILES: Cc1c(-c2ccc(-c3ccc4c(c3)N(C(=O)Nc3nc5ccccc5s3)CCC4)nc2C(=O)O)cnn1CC12CC3(C)CC(C)(C1)CC(OCCN(C)C(=O)CCOCCOCCOCCOCCOCCOCCOCCOCCNC(=O)CCN1C(=O)C=CC1=O)(C3)C2] |
| 2.16 | KZ | 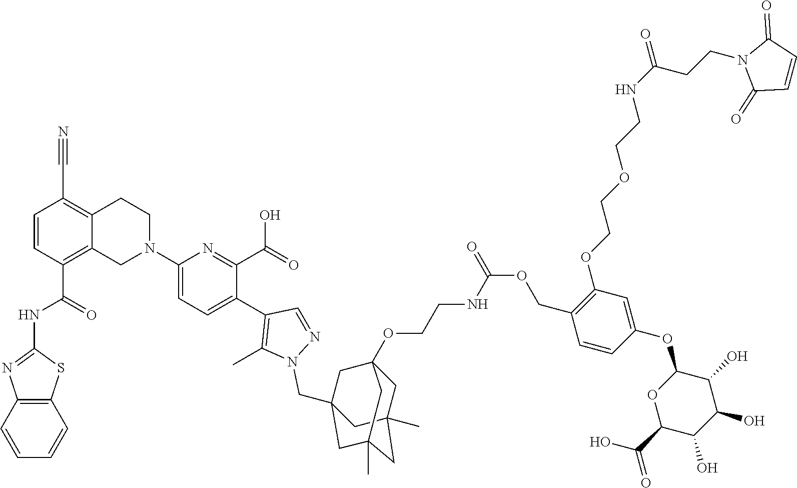 [SMILES: Cc1c(-c2ccc(N3CCc4c(C#N)ccc(C(=O)Nc5nc6ccccc6s5)c4C3)nc2C(=O)O)cnn1CC12CC3(C)CC(C)(C1)CC(OCCNC(=O)OCc1ccc(O[C@@H]4O[C@H](C(=O)O)[C@@H](O)[C@H](O)[C@H]4O)cc1OCCOCCNC(=O)CCN1C(=O)C=CC1=O)(C3)C2] |
| 2.17 | LW | 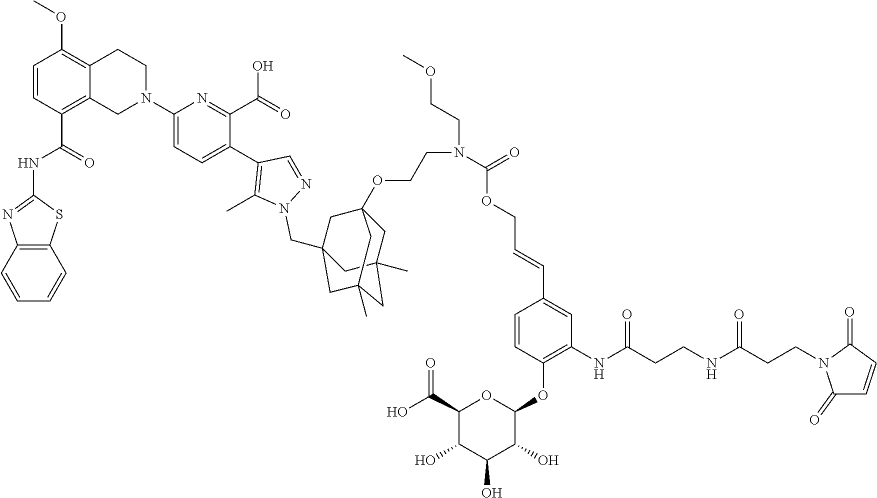 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OC/C=C/c1ccc(O[C@@H]2O[C@H](C(=O)O)[C@@H](O)[C@H](O)[C@H]2O)c(NC(=O)CCNC(=O)CCN2C(=O)C=CC2=O)c1] |
| 2.18 | LY | 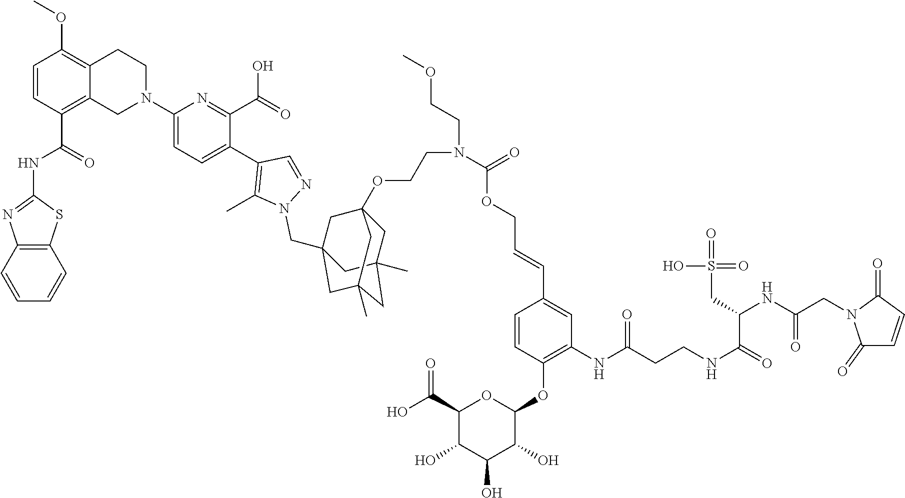 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OC/C=C/c1ccc(O[C@@H]2O[C@H](C(=O)O)[C@@H](O)[C@H](O)[C@H]2O)c(NC(=O)CCNC(=O)[C@H](CS(=O)(=O)O)NC(=O)CN2C(=O)C=CC2=O)c1] |
| 2.19 | LZ | 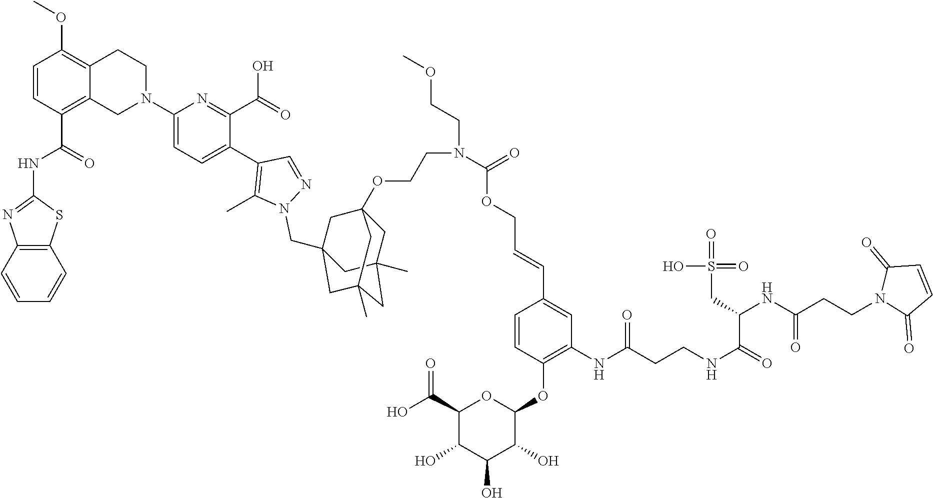 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OC/C=C/c1ccc(O[C@@H]2O[C@H](C(=O)O)[C@@H](O)[C@H](O)[C@H]2O)c(NC(=O)CCNC(=O)[C@H](CS(=O)(=O)O)NC(=O)CCN2C(=O)C=CC2=O)c1] |
| 2.20 | MB | 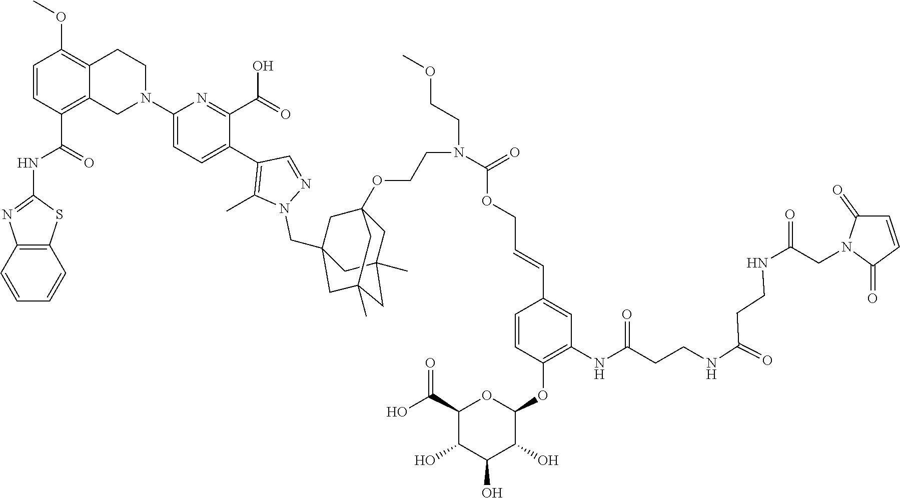 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OC/C=C/c1ccc(O[C@@H]2O[C@H](C(=O)O)[C@@H](O)[C@H](O)[C@H]2O)c(NC(=O)CCNC(=O)CCNC(=O)CN2C(=O)C=CC2=O)c1] |
| 2.21 | MC | 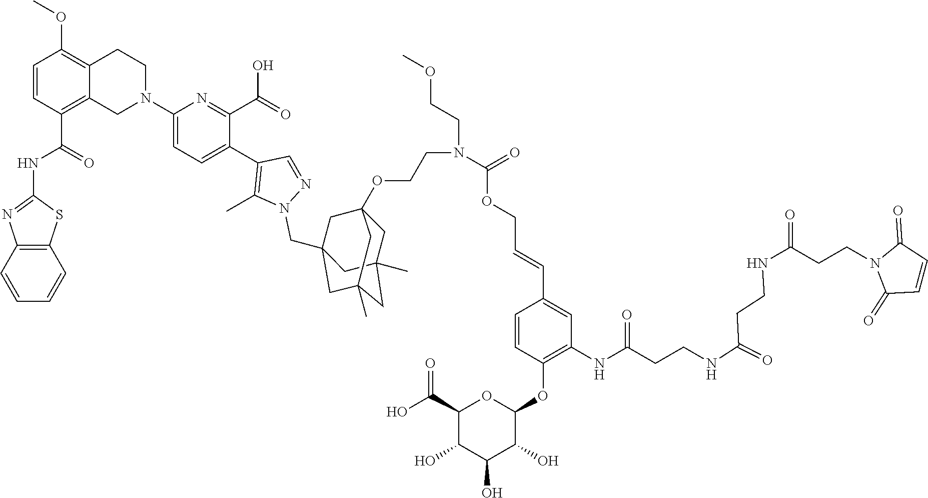 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OC/C=C/c1ccc(O[C@@H]2O[C@H](C(=O)O)[C@@H](O)[C@H](O)[C@H]2O)c(NC(=O)CCNC(=O)CCNC(=O)CCN2C(=O)C=CC2=O)c1] |
| 2.22 | ME | 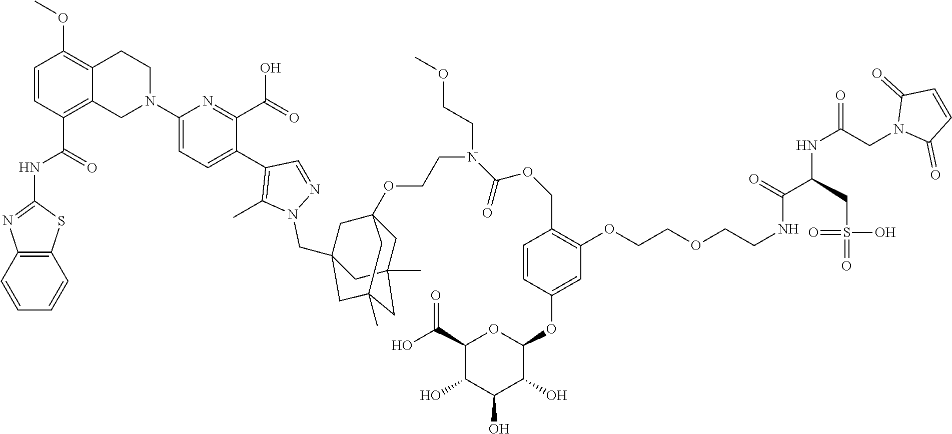 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OCc1ccc(O[C@@H]2O[C@H](C(=O)O)[C@@H](O)[C@H](O)[C@H]2O)cc1OCCOCCNC(=O)[C@H](CS(=O)(=O)O)NC(=O)CN1C(=O)C=CC1=O] |
| 2.23 | MF | 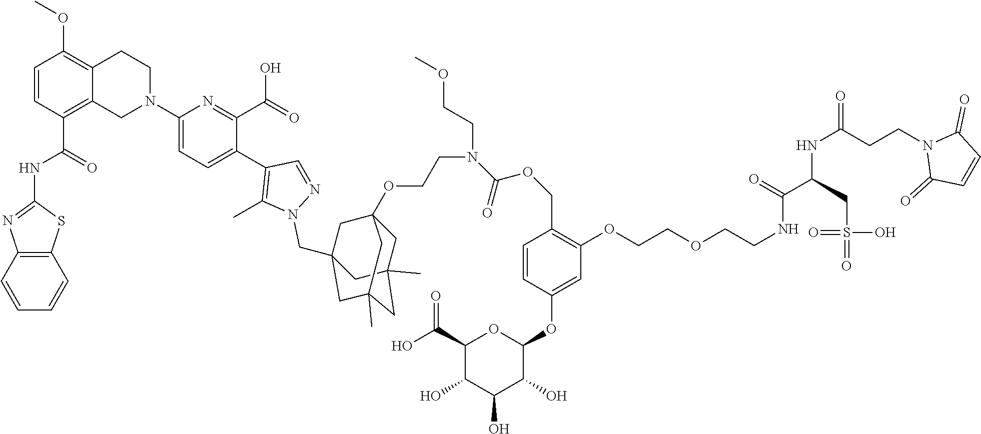 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OCc1ccc(O[C@@H]2O[C@H](C(=O)O)[C@@H](O)[C@H](O)[C@H]2O)cc1OCCOCCNC(=O)[C@H](CS(=O)(=O)O)NC(=O)CCN1C(=O)C=CC1=O] |
| 2.24 | MH | 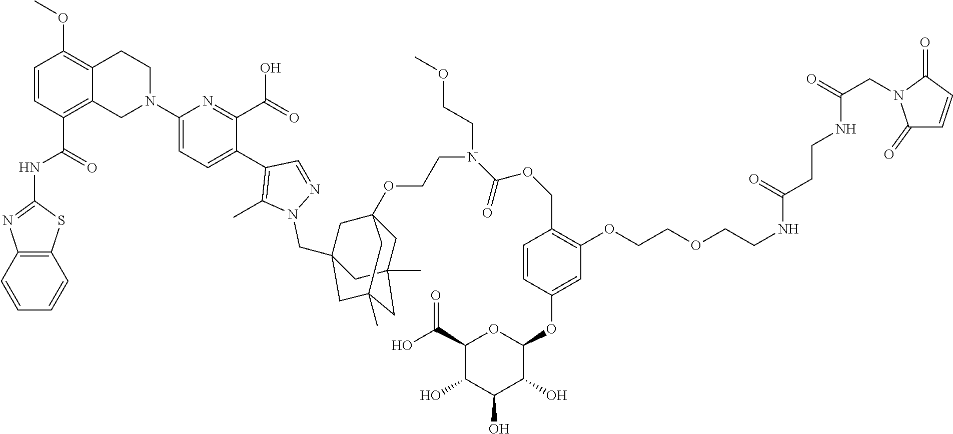 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OCc1ccc(O[C@@H]2O[C@H](C(=O)O)[C@@H](O)[C@H](O)[C@H]2O)cc1OCCOCCNC(=O)CCNC(=O)CN1C(=O)C=CC1=O] |
| 2.25 | MI | 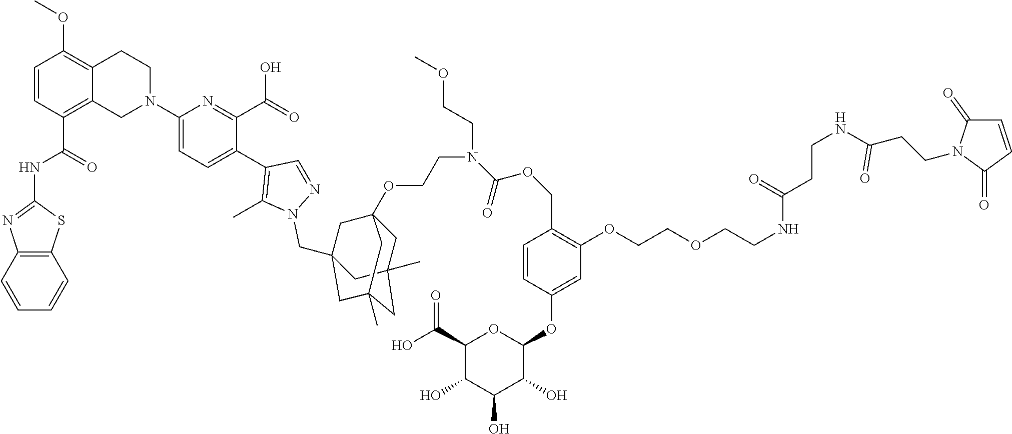 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OCc1ccc(O[C@@H]2O[C@H](C(=O)O)[C@@H](O)[C@H](O)[C@H]2O)cc1OCCOCCNC(=O)CCNC(=O)CCN1C(=O)C=CC1=O] |
| 2.26 | NJ | 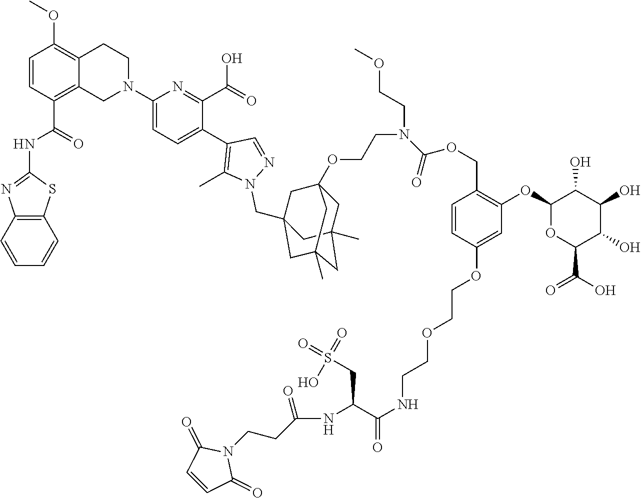 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OCc1ccc(OCCOCCNC(=O)[C@H](CS(=O)(=O)O)NC(=O)CCN2C(=O)C=CC2=O)cc1O[C@@H]1O[C@H](C(=O)O)[C@@H](O)[C@H](O)[C@H]1O] |
| 2.27 | NK | 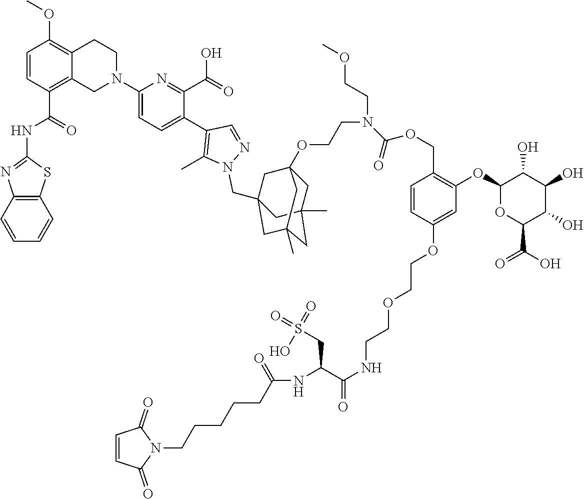 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OCc1ccc(OCCOCCNC(=O)[C@H](CS(=O)(=O)O)NC(=O)CCCCCN2C(=O)C=CC2=O)cc1O[C@@H]1O[C@H](C(=O)O)[C@@H](O)[C@H](O)[C@H]1O] |
| 2.28 | NL | 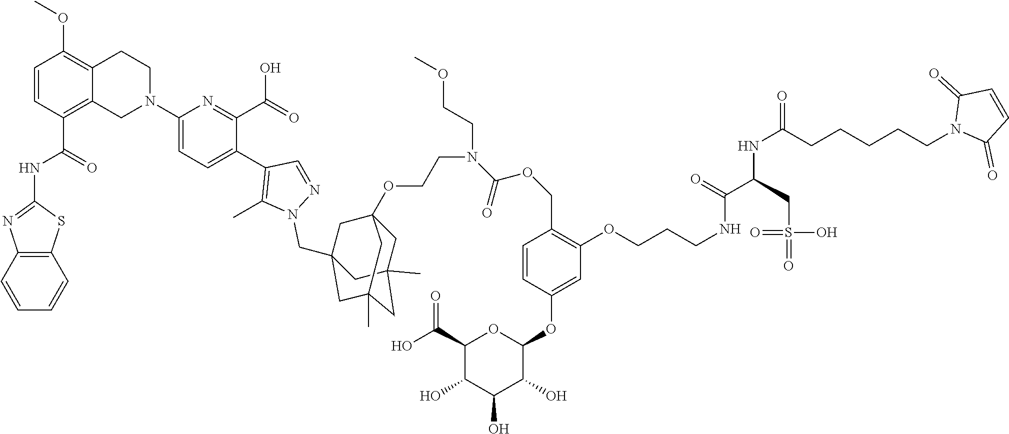 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OCc1ccc(O[C@@H]2O[C@H](C(=O)O)[C@@H](O)[C@H](O)[C@H]2O)cc1OCCCNC(=O)[C@H](CS(=O)(=O)O)NC(=O)CCCCCN1C(=O)C=CC1=O] |
| 2.29 | NM | 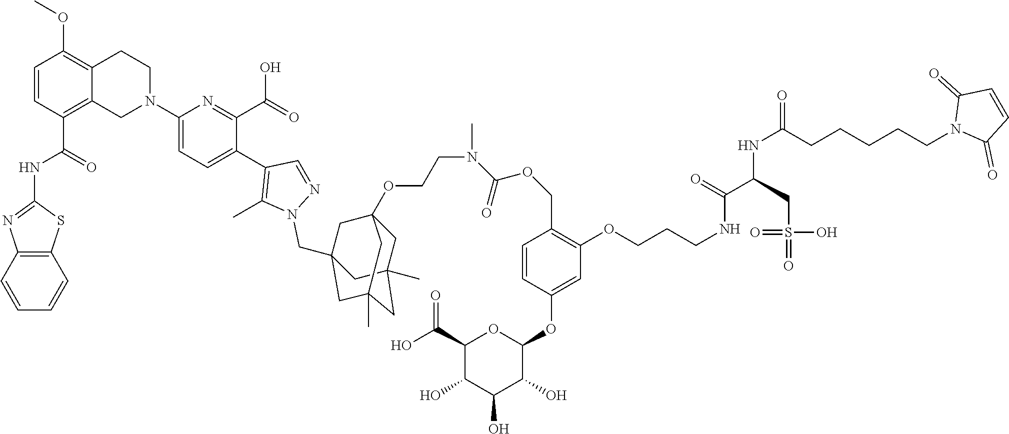 [SMILES: COc1ccc(C(=O)Nc2nc3ccccc3s2)c2c1CCN(c1ccc(-c3cnn(CC45CC6(C)CC(C)(C4)CC(OCCN(C)C(=O)OCc4ccc(O[C@@H]7O[C@H](C(=O)O)[C@@H](O)[C@H](O)[C@H]7O)cc4OCCCNC(=O)[C@H](CS(=O)(=O)O)NC(=O)CCCCCN4C(=O)C=CC4=O)(C6)C5)c3C)c(C(=O)O)n1)C2] |
| 2.30 | NR | 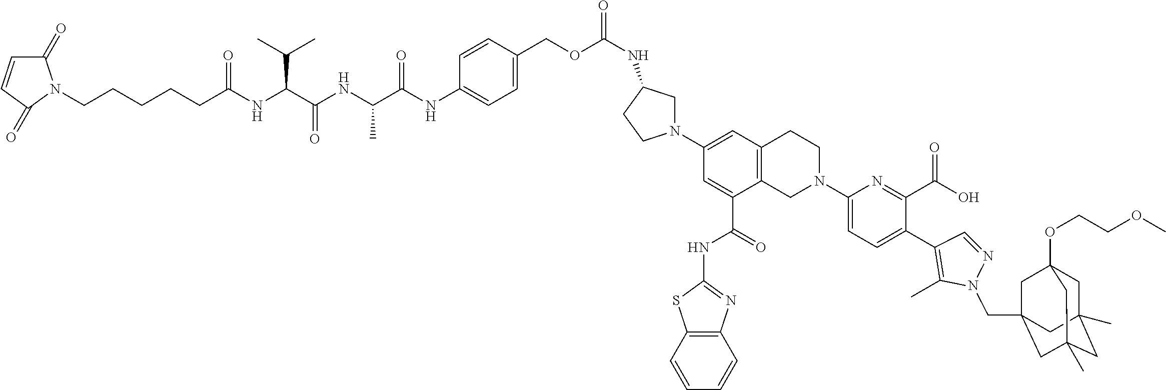 [SMILES: COCCOC12CC3(C)CC(C)(CC(Cn4ncc(-c5ccc(N6CCc7cc(N8CC[C@H](NC(=O)OCc9ccc(NC(=O)[C@H](C)NC(=O)[C@@H](NC(=O)CCCCCN%10C(=O)C=CC%10=O)C(C)C)cc9)C8)cc(C(=O)Nc8nc9ccccc9s8)c7C6)nc5C(=O)O)c4C)(C3)C1)C2] |
| 2.31 | EB | 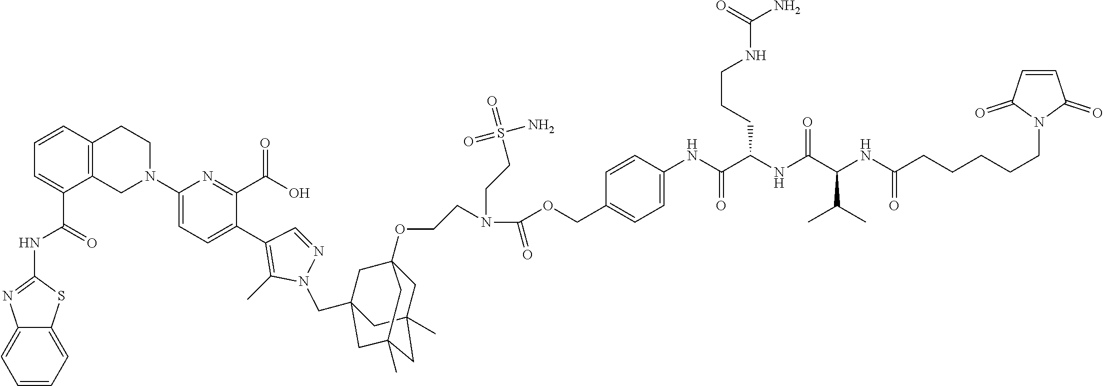 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(N)(=O)=O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.34 | OG | 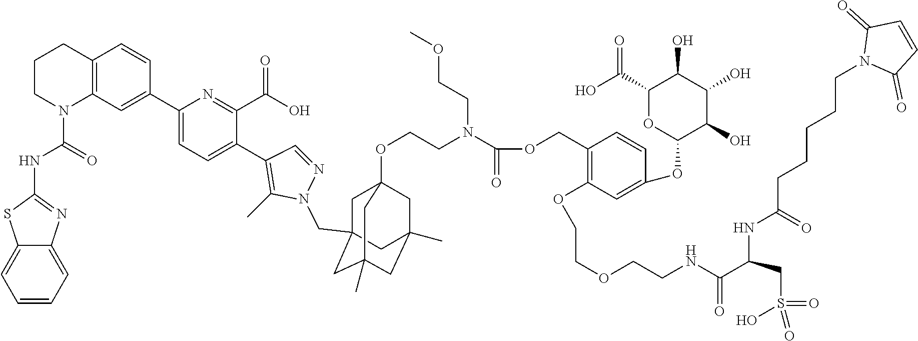 [SMILES: COCCN(CCOC12CC3(C)CC(C)(CC(Cn4ncc(-c5ccc(-c6ccc7c(c6)N(C(=O)Nc6nc8ccccc8s6)CCC7)nc5C(=O)O)c4C)(C3)C1)C2)C(=O)OCc1ccc(O[C@@H]2O[C@H](C(=O)O)[C@@H](O)[C@H](O)[C@H]2O)cc1OCCOCCNC(=O)[C@H](CS(=O)(=O)O)NC(=O)CCCCCN1C(=O)C=CC1=O] |
| 2.35 | OH | 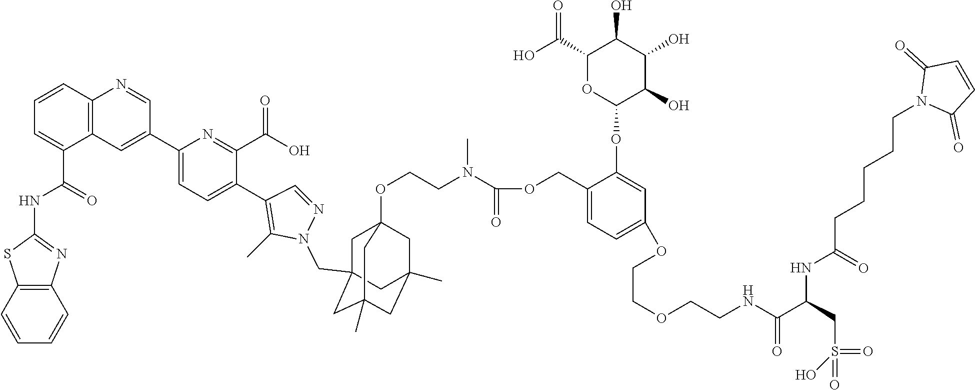 [SMILES: Cc1c(-c2ccc(-c3cnc4cccc(C(=O)Nc5nc6ccccc6s5)c4c3)nc2C(=O)O)cnn1CC12CC3(C)CC(C)(C1)CC(OCCN(C)C(=O)OCc1ccc(OCCOCCNC(=O)[C@H](CS(=O)(=O)O)NC(=O)CCCCCN4C(=O)C=CC4=O)cc1O[C@@H]1O[C@H](C(=O)O)[C@@H](O)[C@H](O)[C@H]1O)(C3)C2] |
| 2.36 | ON | 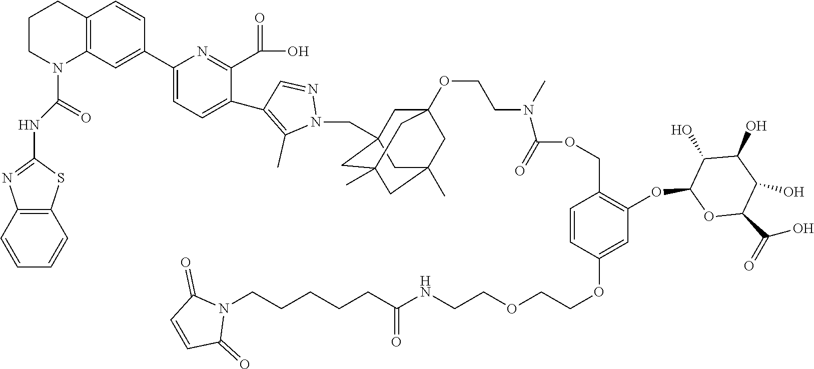 [SMILES: Cc1c(-c2ccc(-c3ccc4c(c3)N(C(=O)Nc3nc5ccccc5s3)CCC4)nc2C(=O)O)cnn1CC12CC3(C)CC(C)(C1)CC(OCCN(C)C(=O)OCc1ccc(OCCOCCNC(=O)CCCCCN4C(=O)C=CC4=O)cc1O[C@@H]1O[C@H](C(=O)O)[C@@H](O)[C@H](O)[C@H]1O)(C3)C2] |
| 2.37 | OT | 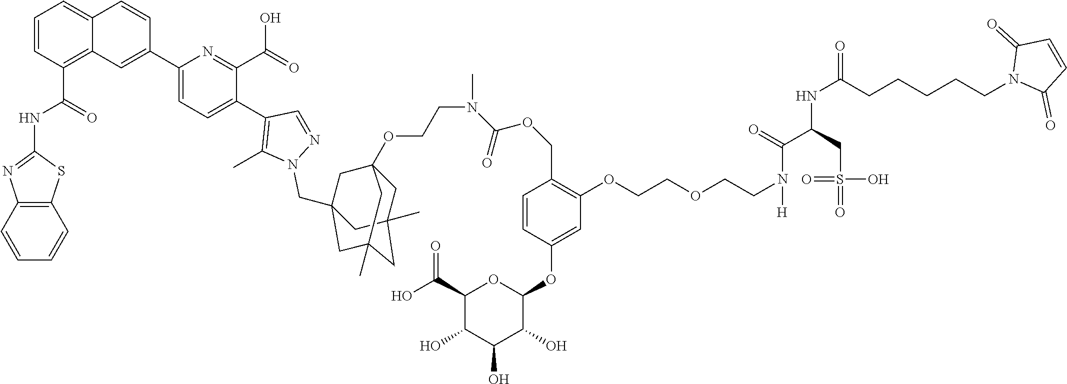 [SMILES: Cc1c(-c2ccc(-c3ccc4cccc(C(=O)Nc5nc6ccccc6s5)c4c3)nc2C(=O)O)cnn1CC12CC3(C)CC(C)(C1)CC(OCCN(C)C(=O)OCc1ccc(O[C@@H]4O[C@H](C(=O)O)[C@@H](O)[C@H](O)[C@H]4O)cc1OCCOCCNC(=O)[C@H](CS(=O)(=O)O)NC(=O)CCCCCN1C(=O)C=CC1=O)(C3)C2] |
| 2.38 | OP | 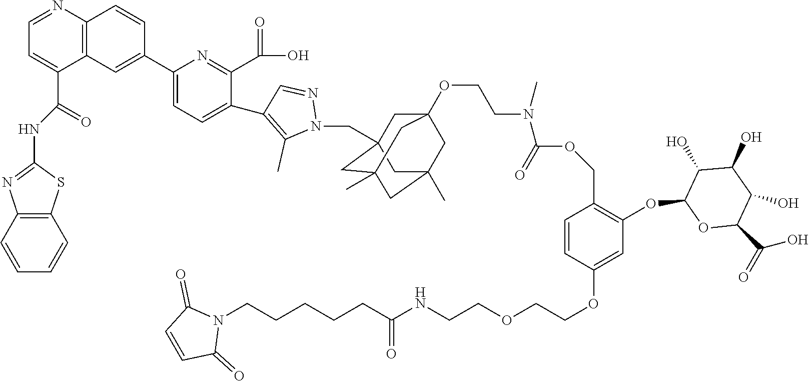 [SMILES: Cc1c(-c2ccc(-c3ccc4nccc(C(=O)Nc5nc6ccccc6s5)c4c3)nc2C(=O)O)cnn1CC12CC3(C)CC(C)(C1)CC(OCCN(C)C(=O)OCc1ccc(OCCOCCNC(=O)CCCCCN4C(=O)C=CC4=O)cc1O[C@@H]1O[C@H](C(=O)O)[C@@H](O)[C@H](O)[C@H]1O)(C3)C2] |
| 2.39 | OU | 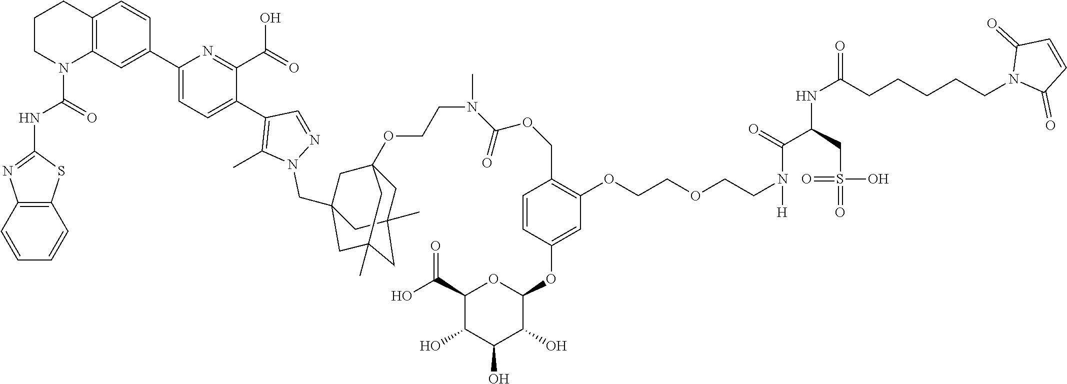 [SMILES: Cc1c(-c2ccc(-c3ccc4c(c3)N(C(=O)Nc3nc5ccccc5s3)CCC4)nc2C(=O)O)cnn1CC12CC3(C)CC(C)(C1)CC(OCCN(C)C(=O)OCc1ccc(O[C@@H]4O[C@H](C(=O)O)[C@@H](O)[C@H](O)[C@H]4O)cc1OCCOCCNC(=O)[C@H](CS(=O)(=O)O)NC(=O)CCCCCN1C(=O)C=CC1=O)(C3)C2] |
| 2.40 | OO | 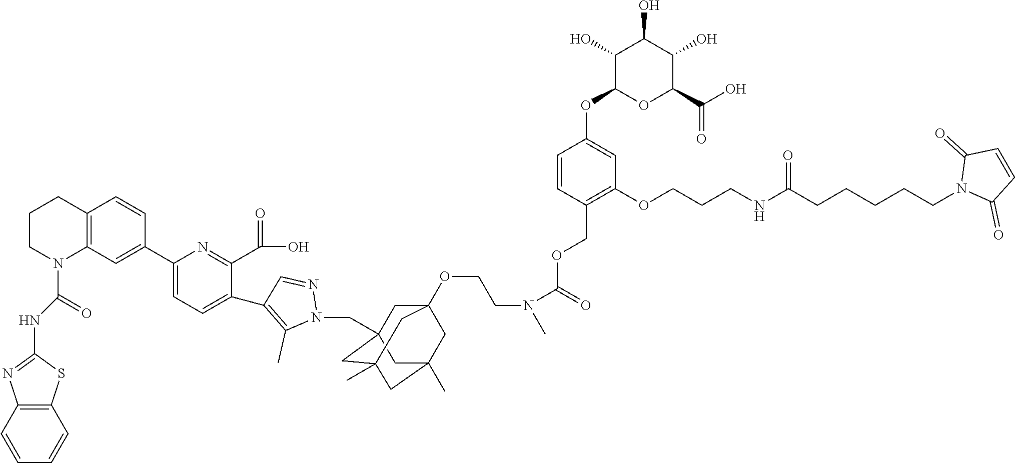 [SMILES: Cc1c(-c2ccc(-c3ccc4c(c3)N(C(=O)Nc3nc5ccccc5s3)CCC4)nc2C(=O)O)cnn1CC12CC3(C)CC(C)(C1)CC(OCCN(C)C(=O)OCc1ccc(O[C@@H]4O[C@H](C(=O)O)[C@@H](O)[C@H](O)[C@H]4O)cc1OCCCNC(=O)CCCCCN1C(=O)C=CC1=O)(C3)C2] |
| 2.41 | OQ | 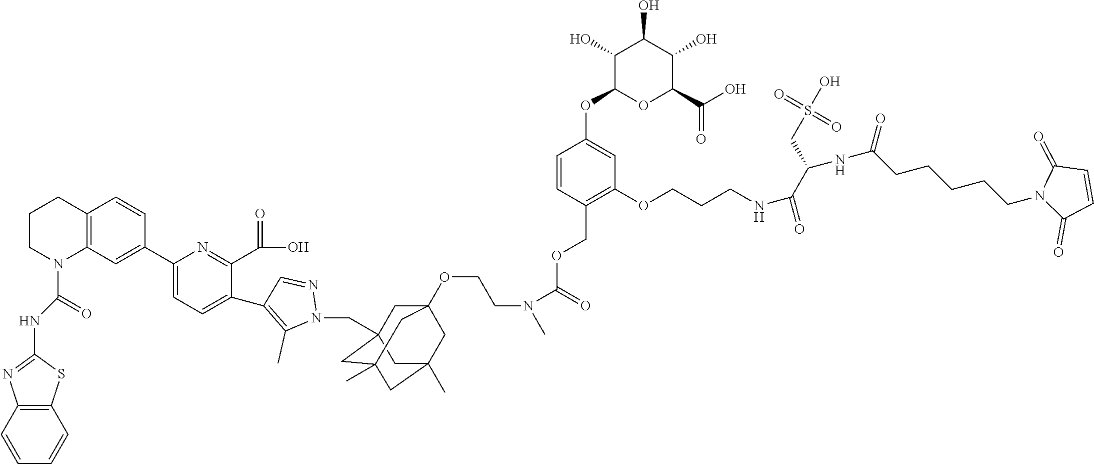 [SMILES: Cc1c(-c2ccc(-c3ccc4c(c3)N(C(=O)Nc3nc5ccccc5s3)CCC4)nc2C(=O)O)cnn1CC12CC3(C)CC(C)(C1)CC(OCCN(C)C(=O)OCc1ccc(O[C@@H]4O[C@H](C(=O)O)[C@@H](O)[C@H](O)[C@H]4O)cc1OCCCNC(=O)[C@H](CS(=O)(=O)O)NC(=O)CCCCCN1C(=O)C=CC1=O)(C3)C2] |
| 2.42 | OR | 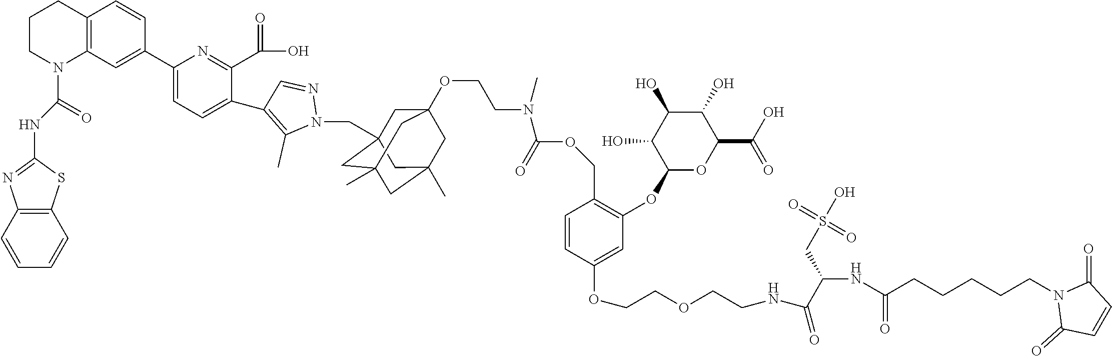 [SMILES: Cc1c(-c2ccc(-c3ccc4c(c3)N(C(=O)Nc3nc5ccccc5s3)CCC4)nc2C(=O)O)cnn1CC12CC3(C)CC(C)(C1)CC(OCCN(C)C(=O)OCc1ccc(OCCOCCNC(=O)[C@H](CS(=O)(=O)O)NC(=O)CCCCCN4C(=O)C=CC4=O)cc1O[C@@H]1O[C@H](C(=O)O)[C@@H](O)[C@H](O)[C@H]1O)(C3)C2] |
| 2.43 | OS | 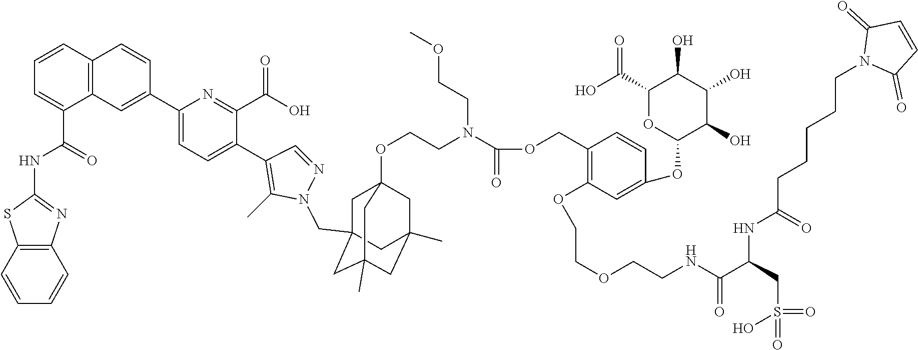 [SMILES: COCCN(CCOC12CC3(C)CC(C)(CC(Cn4ncc(-c5ccc(-c6ccc7cccc(C(=O)Nc8nc9ccccc9s8)c7c6)nc5C(=O)O)c4C)(C3)C1)C2)C(=O)OCc1ccc(O[C@@H]2O[C@H](C(=O)O)[C@@H](O)[C@H](O)[C@H]2O)cc1OCCOCCNC(=O)[C@H](CS(=O)(=O)O)NC(=O)CCCCCN1C(=O)C=CC1=O] |
| 2.44 | OX | 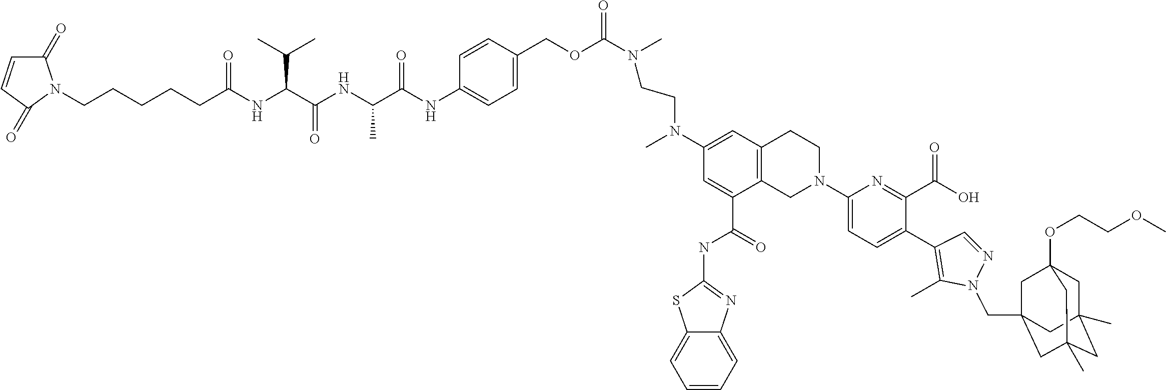 [SMILES: COCCOC12CC3(C)CC(C)(CC(Cn4ncc(-c5ccc(N6CCc7cc(N(C)CCN(C)C(=O)OCc8ccc(NC(=O)[C@H](C)NC(=O)[C@@H](NC(=O)CCCCCN9C(=O)C=CC9=O)C(C)C)cc8)cc(C(=O)Nc8nc9ccccc9s8)c7C6)nc5C(=O)O)c4C)(C3)C1)C2] |
| 2.45 | OZ | 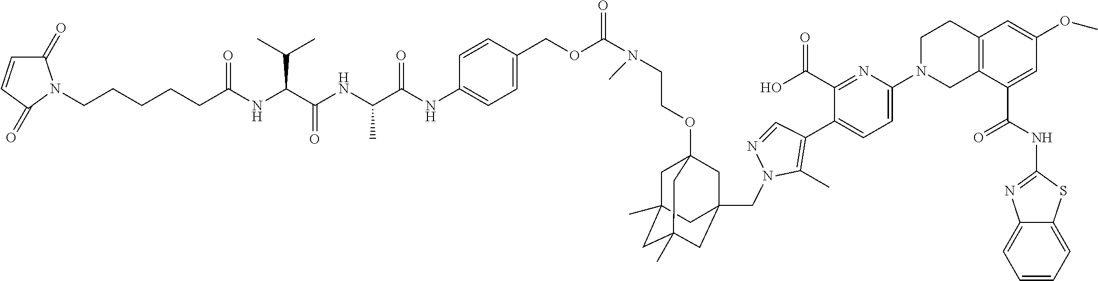 [SMILES: COc1cc2c(c(C(=O)Nc3nc4ccccc4s3)c1)CN(c1ccc(-c3cnn(CC45CC6(C)CC(C)(C4)CC(OCCN(C)C(=O)OCc4ccc(NC(=O)[C@H](C)NC(=O)[C@@H](NC(=O)CCCCCN7C(=O)C=CC7=O)C(C)C)cc4)(C6)C5)c3C)c(C(=O)O)n1)CC2] |
| 2.46 | PA | 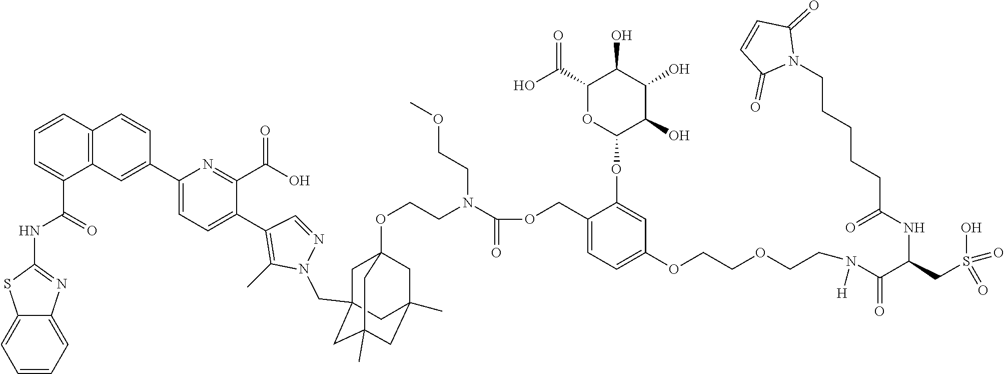 [SMILES: COCCN(CCOC12CC3(C)CC(C)(CC(Cn4ncc(-c5ccc(-c6ccc7cccc(C(=O)Nc8nc9ccccc9s8)c7c6)nc5C(=O)O)c4C)(C3)C1)C2)C(=O)OCc1ccc(OCCOCCNC(=O)[C@H](CS(=O)(=O)O)NC(=O)CCCCCN2C(=O)C=CC2=O)cc1O[C@@H]1O[C@H](C(=O)O)[C@@H](O)[C@H](O)[C@H]1O] |
| 2.47 | QL | 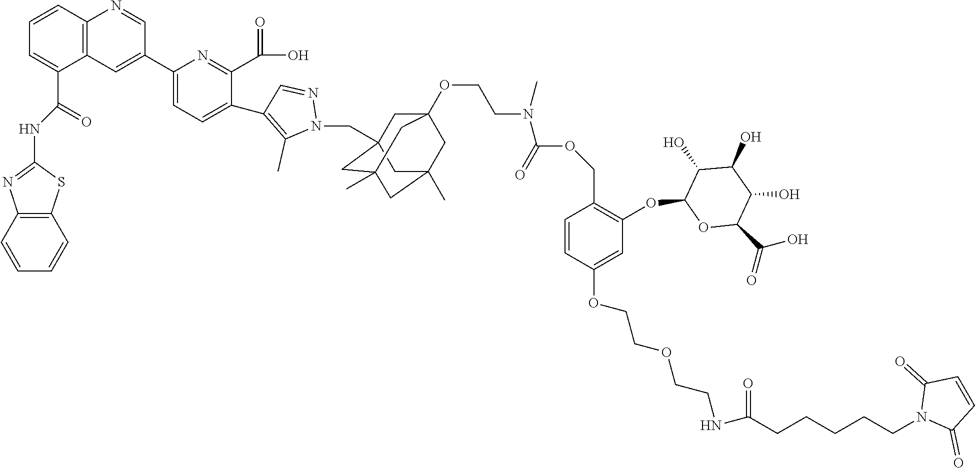 [SMILES: Cc1c(-c2ccc(-c3cnc4cccc(C(=O)Nc5nc6ccccc6s5)c4c3)nc2C(=O)O)cnn1CC12CC3(C)CC(C)(C1)CC(OCCN(C)C(=O)OCc1ccc(OCCOCCNC(=O)CCCCCN4C(=O)C=CC4=O)cc1O[C@@H]1O[C@H](C(=O)O)[C@@H](O)[C@H](O)[C@H]1O)(C3)C2] |
| 2.48 | QM | 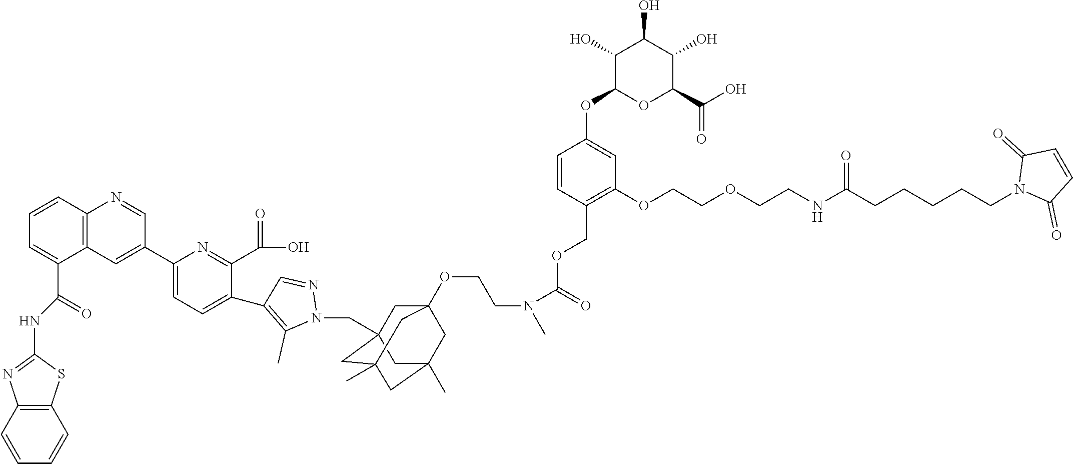 [SMILES: Cc1c(-c2ccc(-c3cnc4cccc(C(=O)Nc5nc6ccccc6s5)c4c3)nc2C(=O)O)cnn1CC12CC3(C)CC(C)(C1)CC(OCCN(C)C(=O)OCc1ccc(O[C@@H]4O[C@H](C(=O)O)[C@@H](O)[C@H](O)[C@H]4O)cc1OCCOCCNC(=O)CCCCCN1C(=O)C=CC1=O)(C3)C2] |
| 2.49 | QN | 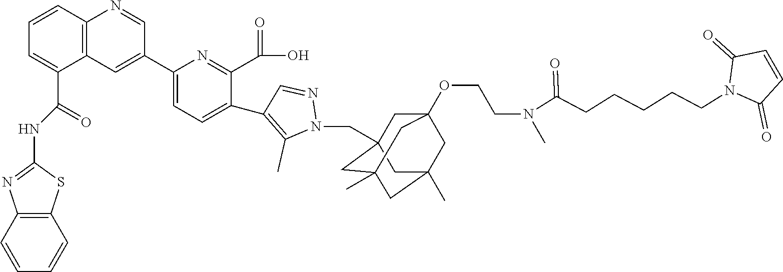 [SMILES: Cc1c(-c2ccc(-c3cnc4cccc(C(=O)Nc5nc6ccccc6s5)c4c3)nc2C(=O)O)cnn1CC12CC3(C)CC(C)(C1)CC(OCCN(C)C(=O)CCCCCN1C(=O)C=CC1=O)(C3)C2] |
| 2.50 | QT | 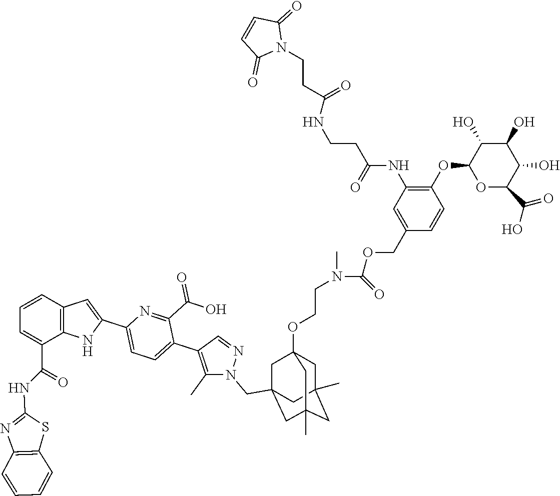 [SMILES: Cc1c(-c2ccc(-c3cc4cccc(C(=O)Nc5nc6ccccc6s5)c4[nH]3)nc2C(=O)O)cnn1CC12CC3(C)CC(C)(C1)CC(OCCN(C)C(=O)OCc1ccc(O[C@@H]4O[C@H](C(=O)O)[C@@H](O)[C@H](O)[C@H]4O)c(NC(=O)CCNC(=O)CCN4C(=O)C=CC4=O)c1)(C3)C2] |
| 2.51 | RF | 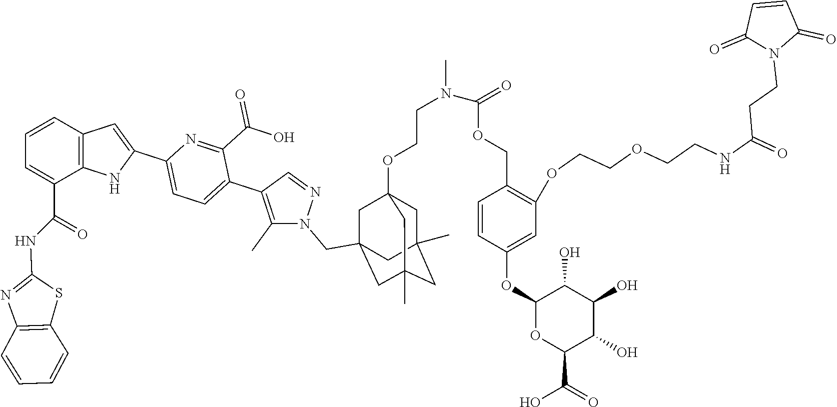 [SMILES: Cc1c(-c2ccc(-c3cc4cccc(C(=O)Nc5nc6ccccc6s5)c4[nH]3)nc2C(=O)O)cnn1CC12CC3(C)CC(C)(C1)CC(OCCN(C)C(=O)OCc1ccc(O[C@@H]4O[C@H](C(=O)O)[C@@H](O)[C@H](O)[C@H]4O)cc1OCCOCCNC(=O)CCN1C(=O)C=CC1=O)(C3)C2] |
| 2.52 | RG | 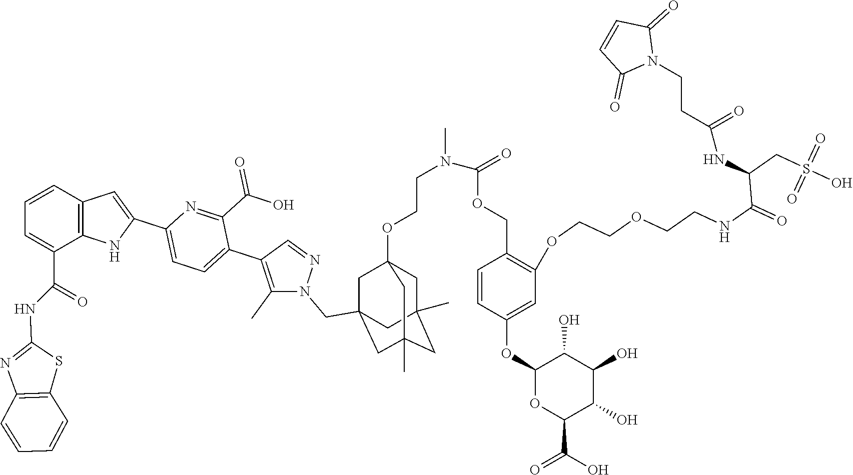 [SMILES: Cc1c(-c2ccc(-c3cc4cccc(C(=O)Nc5nc6ccccc6s5)c4[nH]3)nc2C(=O)O)cnn1CC12CC3(C)CC(C)(C1)CC(OCCN(C)C(=O)OCc1ccc(O[C@@H]4O[C@H](C(=O)O)[C@@H](O)[C@H](O)[C@H]4O)cc1OCCOCCNC(=O)[C@H](CS(=O)(=O)O)NC(=O)CCN1C(=O)C=CC1=O)(C3)C2] |
| 2.53 | SF | 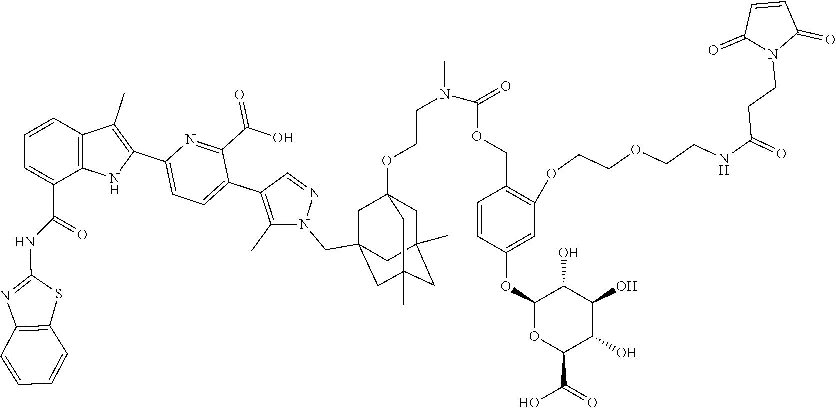 [SMILES: Cc1c(-c2ccc(-c3cnn(CC45CC6(C)CC(C)(C4)CC(OCCN(C)C(=O)OCc4ccc(O[C@@H]7O[C@H](C(=O)O)[C@@H](O)[C@H](O)[C@H]7O)cc4OCCOCCNC(=O)CCN4C(=O)C=CC4=O)(C6)C5)c3C)c(C(=O)O)n2)[nH]c2c(C(=O)Nc3nc4ccccc4s3)cccc12] |
| 2.54 | SR | 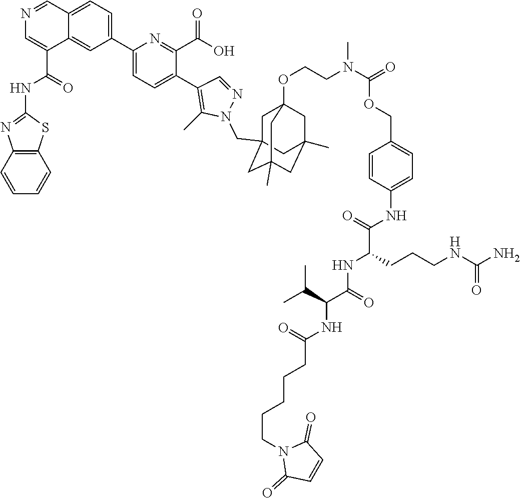 [SMILES: Cc1c(-c2ccc(-c3ccc4cncc(C(=O)Nc5nc6ccccc6s5)c4c3)nc2C(=O)O)cnn1CC12CC3(C)CC(C)(C1)CC(OCCN(C)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.55 | YZ | 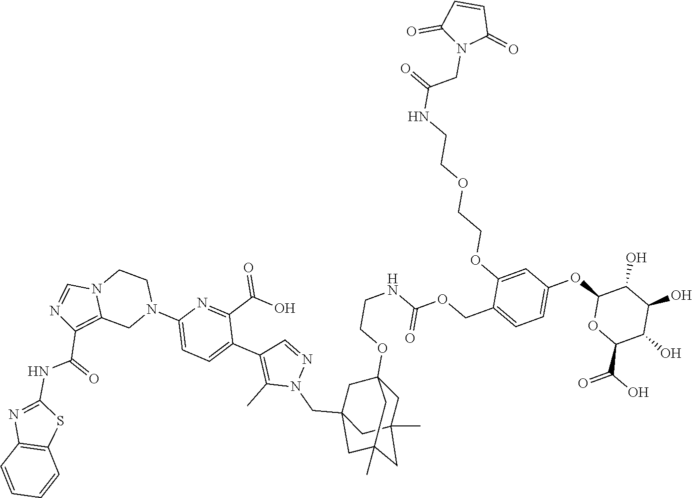 [SMILES: Cc1c(-c2ccc(N3CCn4cnc(C(=O)Nc5nc6ccccc6s5)c4C3)nc2C(=O)O)cnn1CC12CC3(C)CC(C)(C1)CC(OCCNC(=O)OCc1ccc(O[C@@H]4O[C@H](C(=O)O)[C@@H](O)[C@H](O)[C@H]4O)cc1OCCOCCNC(=O)CN1C(=O)C=CC1=O)(C3)C2] |
| 2.56 | QR | 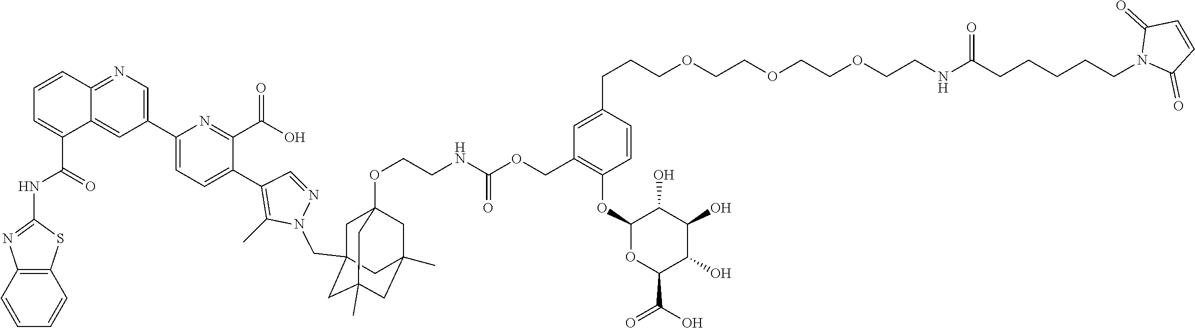 [SMILES: Cc1c(-c2ccc(-c3cnc4cccc(C(=O)Nc5nc6ccccc6s5)c4c3)nc2C(=O)O)cnn1CC12CC3(C)CC(C)(C1)CC(OCCNC(=O)OCc1cc(CCCOCCOCCOCCNC(=O)CCCCCN4C(=O)C=CC4=O)ccc1O[C@@H]1O[C@H](C(=O)O)[C@@H](O)[C@H](O)[C@H]1O)(C3)C2] |
| 2.57 | SE | 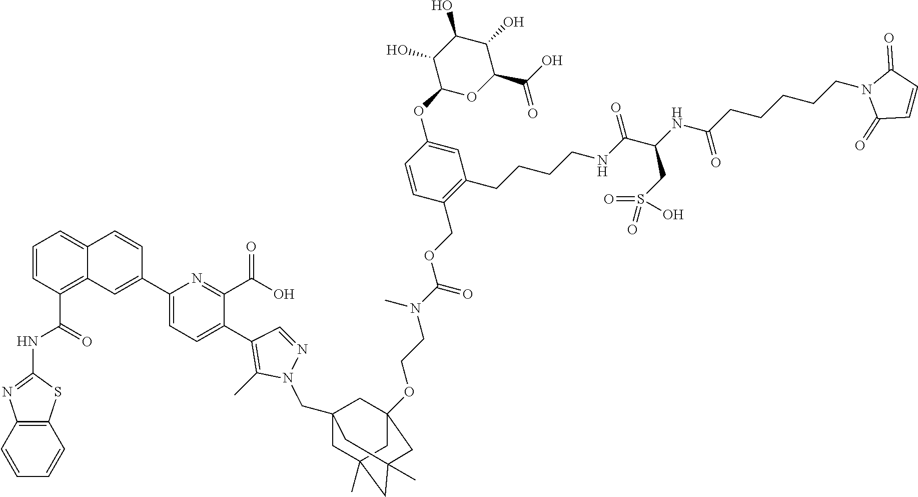 [SMILES: Cc1c(-c2ccc(-c3ccc4cccc(C(=O)Nc5nc6ccccc6s5)c4c3)nc2C(=O)O)cnn1CC12CC3(C)CC(C)(C1)CC(OCCN(C)C(=O)OCc1ccc(O[C@@H]4O[C@H](C(=O)O)[C@@H](O)[C@H](O)[C@H]4O)cc1CCCCNC(=O)[C@H](CS(=O)(=O)O)NC(=O)CCCCCN1C(=O)C=CC1=O)(C3)C2] |
| 2.58 | UH | 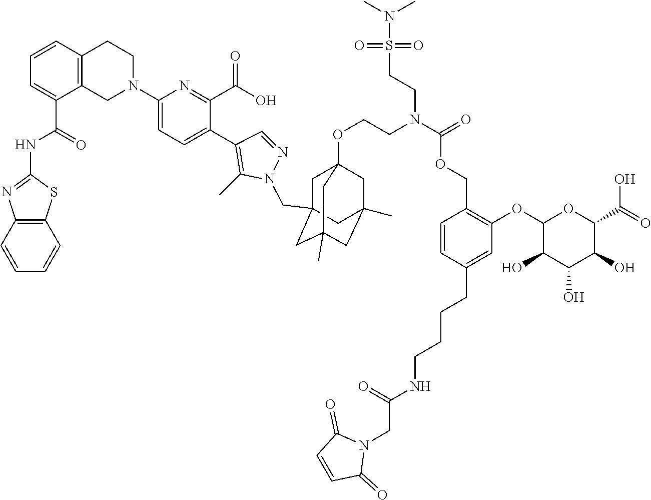 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)N(C)C)C(=O)OCc1ccc(CCCCNC(=O)CN4C(=O)C=CC4=O)cc1OC1O[C@H](C(=O)O)[C@@H](O)[C@H](O)[C@H]1O)(C3)C2] |
| 2.59 | UI | 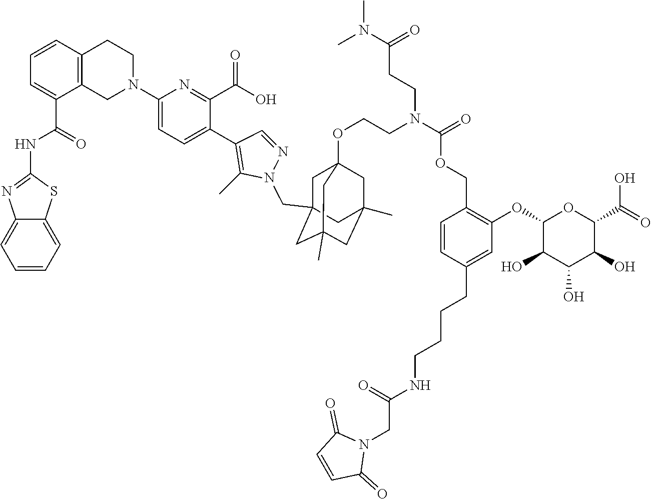 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCC(=O)N(C)C)C(=O)OCc1ccc(CCCCNC(=O)CN4C(=O)C=CC4=O)cc1O[C@@H]1O[C@H](C(=O)O)[C@@H](O)[C@H](O)[C@H]1O)(C3)C2] |
| 2.60 | US | 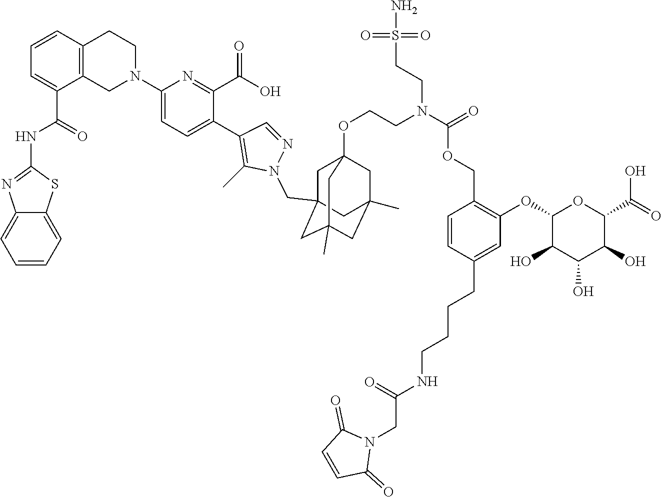 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(N)(=O)=O)C(=O)OCc1ccc(CCCCNC(=O)CN4C(=O)C=CC4=O)cc1O[C@@H]1O[C@H](C(=O)O)[C@@H](O)[C@H](O)[C@H]1O)(C3)C2] |
| 2.61 | UY | 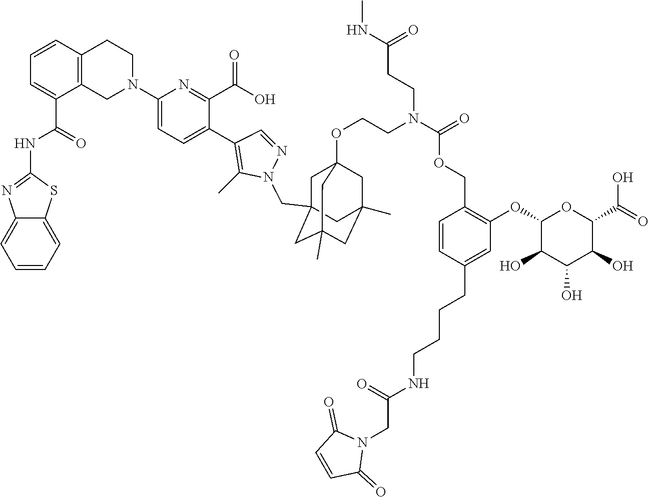 [SMILES: CNC(=O)CCN(CCOC12CC3(C)CC(C)(CC(Cn4ncc(-c5ccc(N6CCc7cccc(C(=O)Nc8nc9ccccc9s8)c7C6)nc5C(=O)O)c4C)(C3)C1)C2)C(=O)OCc1ccc(CCCCNC(=O)CN2C(=O)C=CC2=O)cc1O[C@@H]1O[C@H](C(=O)O)[C@@H](O)[C@H](O)[C@H]1O] |
| 2.62 | UX | 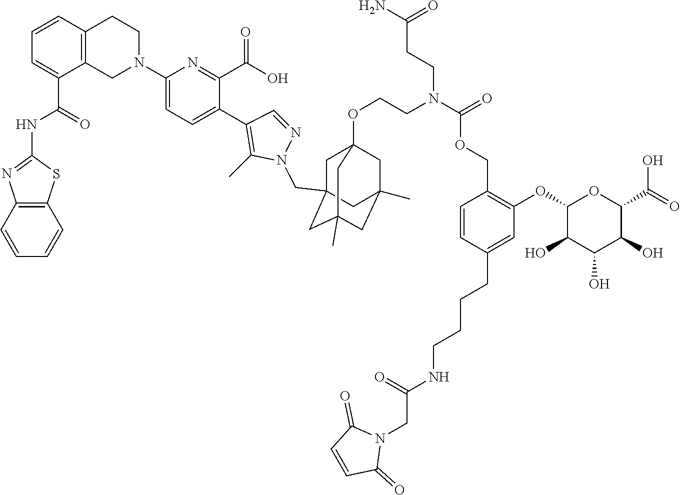 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCC(N)=O)C(=O)OCc1ccc(CCCCNC(=O)CN4C(=O)C=CC4=O)cc1O[C@@H]1O[C@H](C(=O)O)[C@@H](O)[C@H](O)[C@H]1O)(C3)C2] |
| 2.63 | WZ | 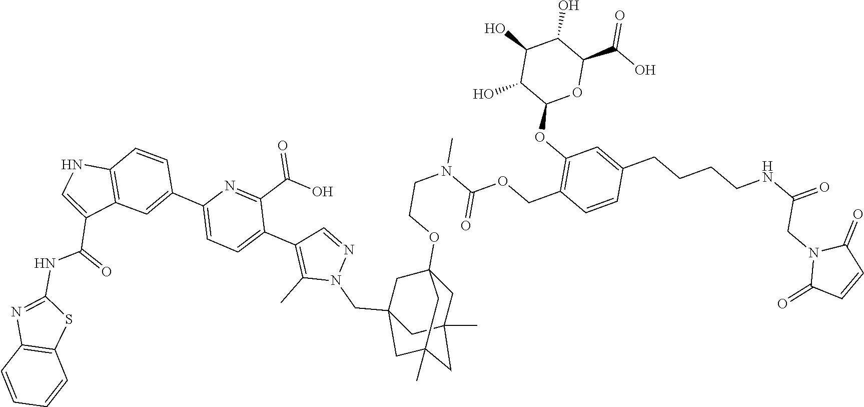 [SMILES: Cc1c(-c2ccc(-c3ccc4[nH]cc(C(=O)Nc5nc6ccccc6s5)c4c3)nc2C(=O)O)cnn1CC12CC3(C)CC(C)(C1)CC(OCCN(C)C(=O)OCc1ccc(CCCCNC(=O)CN4C(=O)C=CC4=O)cc1O[C@@H]1O[C@H](C(=O)O)[C@@H](O)[C@H](O)[C@H]1O)(C3)C2] |
| 2.64 | XO | 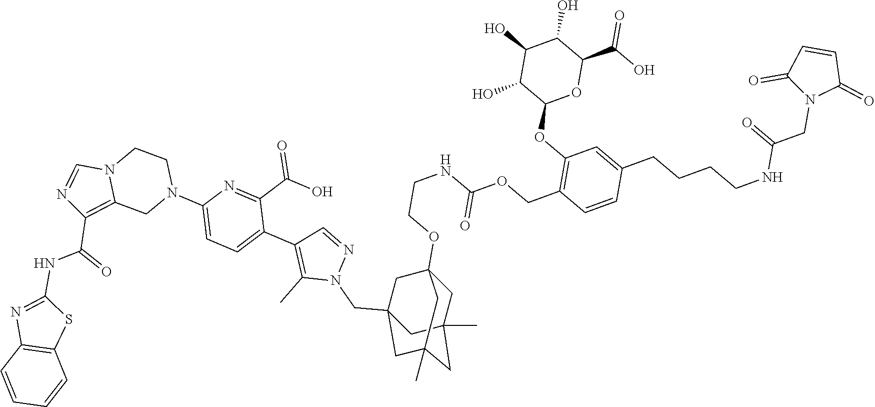 [SMILES: Cc1c(-c2ccc(N3CCn4cnc(C(=O)Nc5nc6ccccc6s5)c4C3)nc2C(=O)O)cnn1CC12CC3(C)CC(C)(C1)CC(OCCNC(=O)OCc1ccc(CCCCNC(=O)CN4C(=O)C=CC4=O)cc1O[C@@H]1O[C@H](C(=O)O)[C@@H](O)[C@H](O)[C@H]1O)(C3)C2] |
| 2.65 | XW | 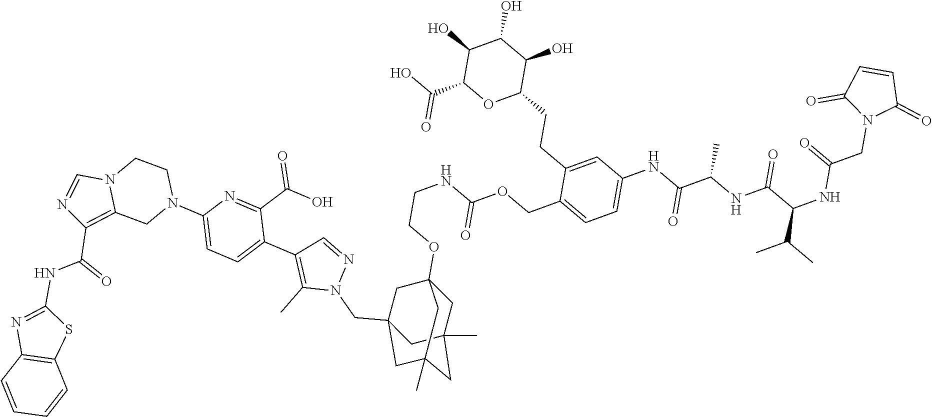 [SMILES: Cc1c(-c2ccc(N3CCn4cnc(C(=O)Nc5nc6ccccc6s5)c4C3)nc2C(=O)O)cnn1CC12CC3(C)CC(C)(C1)CC(OCCNC(=O)OCc1ccc(NC(=O)[C@H](C)NC(=O)[C@@H](NC(=O)CN4C(=O)C=CC4=O)C(C)C)cc1CC[C@@H]1O[C@H](C(=O)O)[C@@H](O)[C@H](O)[C@H]1O)(C3)C2] |
| 2.66 | YG | 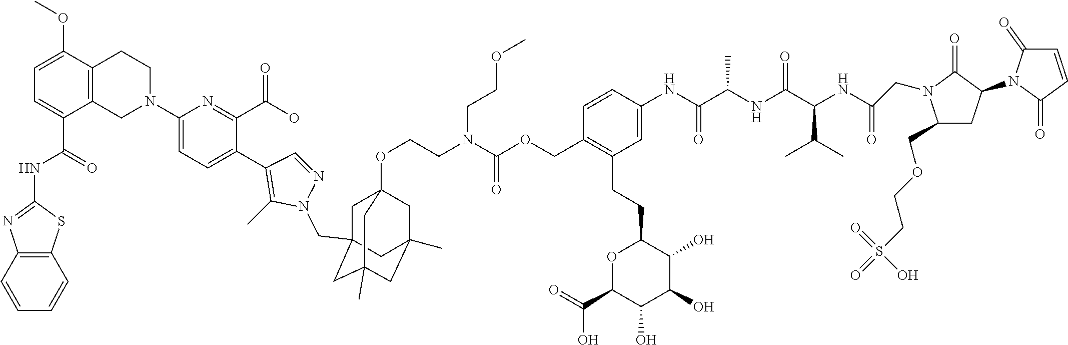 [SMILES: COCCN(CCOC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2)C(=O)OCc1ccc(NC(=O)[C@H](C)NC(=O)[C@@H](NC(=O)CN2C(=O)[C@@H](N3C(=O)C=CC3=O)C[C@H]2COCCS(=O)(=O)O)C(C)C)cc1CC[C@@H]1O[C@H](C(=O)O)[C@@H](O)[C@H](O)[C@H]1O] |
| 2.67 | ZT | 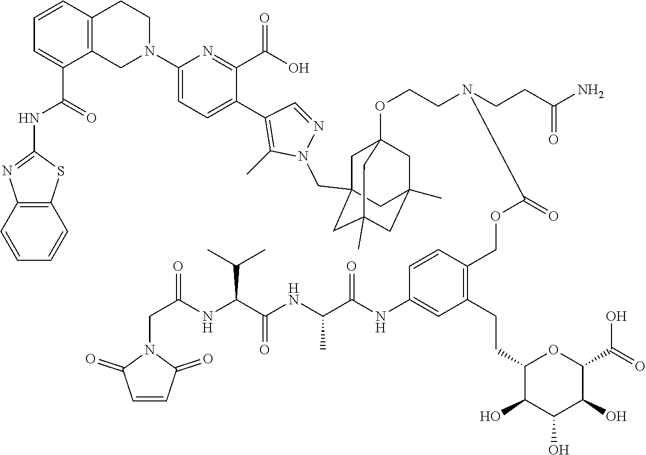 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCC(N)=O)C(=O)OCc1ccc(NC(=O)[C@H](C)NC(=O)[C@@H](NC(=O)CN4C(=O)C=CC4=O)C(C)C)cc1CC[C@@H]1O[C@H](C(=O)O)[C@@H](O)[C@H](O)[C@H]1O)(C3)C2] |
| 2.68 | AAN | 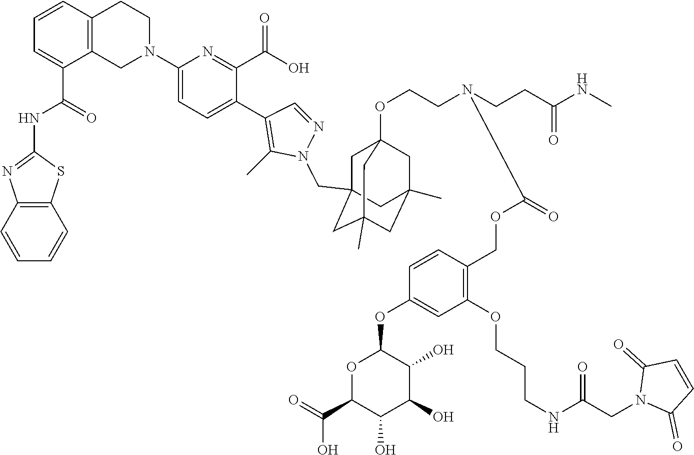 [SMILES: CNC(=O)CCN(CCOC12CC3(C)CC(C)(CC(Cn4ncc(-c5ccc(N6CCc7cccc(C(=O)Nc8nc9ccccc9s8)c7C6)nc5C(=O)O)c4C)(C3)C1)C2)C(=O)OCc1ccc(O[C@@H]2O[C@H](C(=O)O)[C@@H](O)[C@H](O)[C@H]2O)cc1OCCCNC(=O)CN1C(=O)C=CC1=O] |
| 2.69 | AAO | 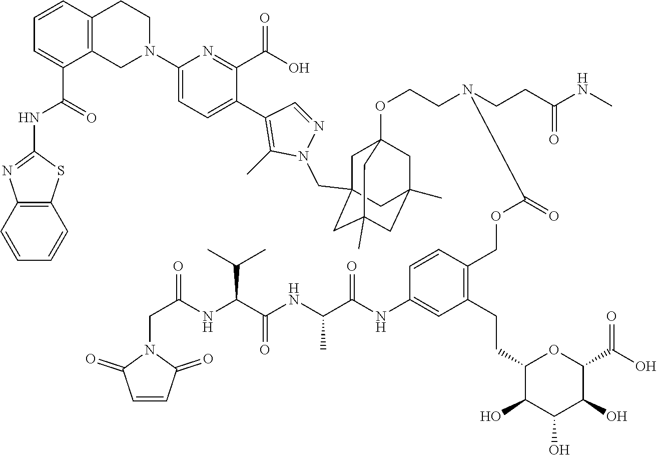 [SMILES: CNC(=O)CCN(CCOC12CC3(C)CC(C)(CC(Cn4ncc(-c5ccc(N6CCc7cccc(C(=O)Nc8nc9ccccc9s8)c7C6)nc5C(=O)O)c4C)(C3)C1)C2)C(=O)OCc1ccc(NC(=O)[C@H](C)NC(=O)[C@@H](NC(=O)CN2C(=O)C=CC2=O)C(C)C)cc1CC[C@@H]1O[C@H](C(=O)O)[C@@H](O)[C@H](O)[C@H]1O] |
| 2.70 | AAP | 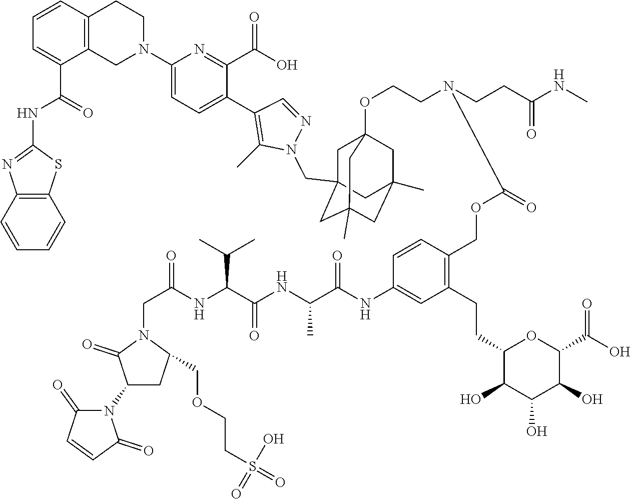 [SMILES: CNC(=O)CCN(CCOC12CC3(C)CC(C)(CC(Cn4ncc(-c5ccc(N6CCc7cccc(C(=O)Nc8nc9ccccc9s8)c7C6)nc5C(=O)O)c4C)(C3)C1)C2)C(=O)OCc1ccc(NC(=O)[C@H](C)NC(=O)[C@@H](NC(=O)CN2C(=O)[C@@H](N3C(=O)C=CC3=O)C[C@H]2COCCS(=O)(=O)O)C(C)C)cc1CC[C@@H]1O[C@H](C(=O)O)[C@@H](O)[C@H](O)[C@H]1O] |
| 2.71 | ABF | 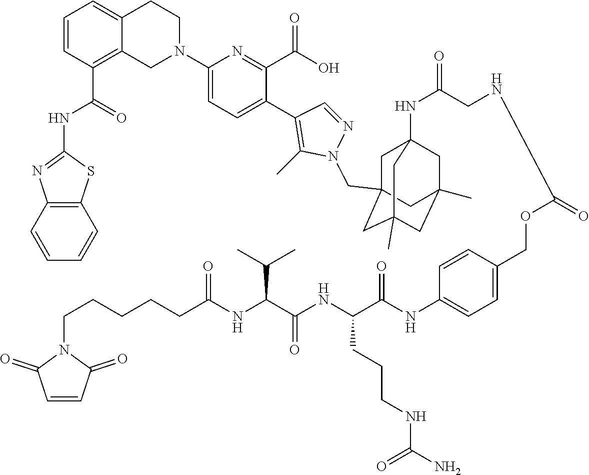 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(NC(=O)CNC(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.72 | ZZ | 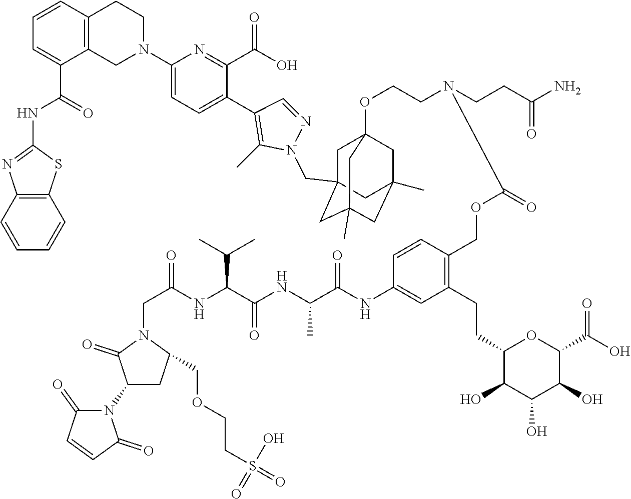 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCC(N)=O)C(=O)OCc1ccc(NC(=O)[C@H](C)NC(=O)[C@@H](NC(=O)CN4C(=O)[C@@H](N5C(=O)C=CC5=O)C[C@H]4COCCS(=O)(=O)O)C(C)C)cc1CC[C@@H]1O[C@H](C(=O)O)[C@@H](O)[C@H](O)[C@H]1O)(C3)C2] |
| 2.73 (con- trol) | CZ | 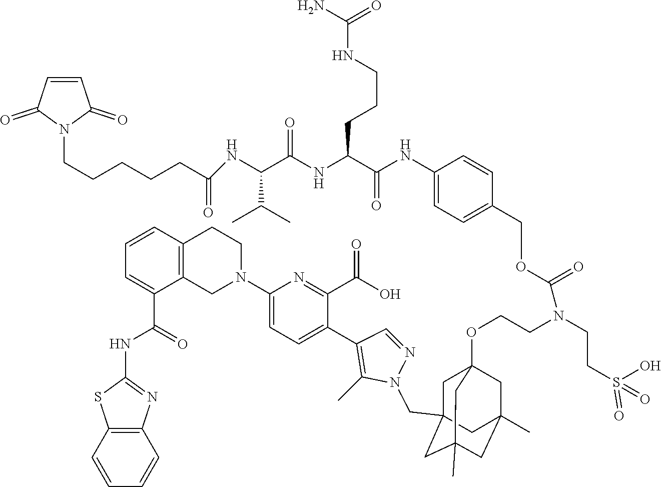 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.74 (con- trol) | TX | 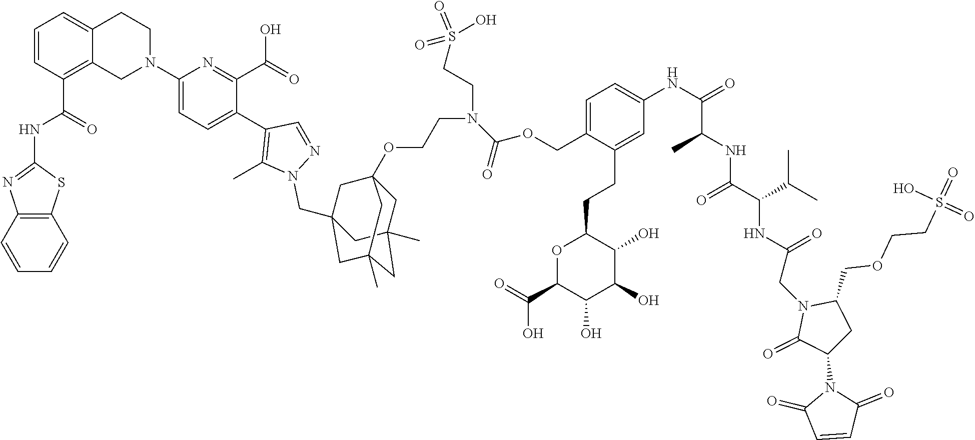 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)OCc1ccc(NC(=O)[C@H](C)NC(=O)[C@@H](NC(=O)CN4C(=O)[C@@H](N5C(=O)C=CC5=O)C[C@H]4COCCS(=O)(=O)O)C(C)C)cc1CC[C@@H]1O[C@H](C(=O)O)[C@@H](O)[C@H](O)[C@H]1O)(C3)C2] |
| 2.75 (con- trol) | LB | 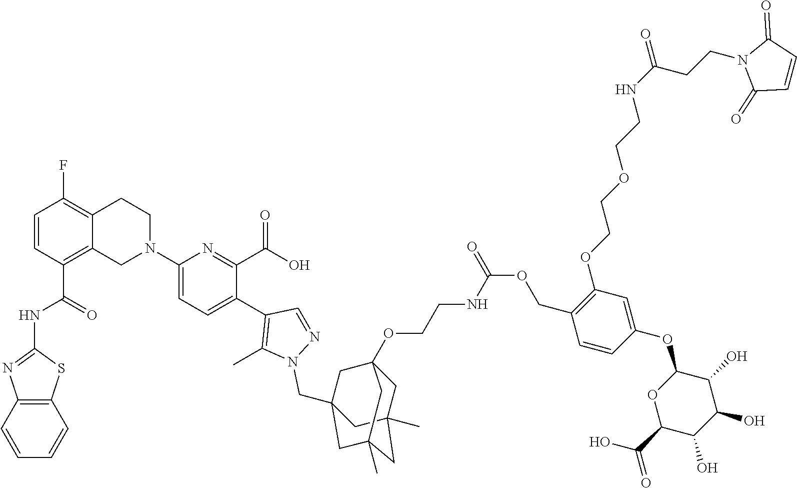 [SMILES: Cc1c(-c2ccc(N3CCc4c(F)ccc(C(=O)Nc5nc6ccccc6s5)c4C3)nc2C(=O)O)cnn1CC12CC3(C)CC(C)(C1)CC(OCCNC(=O)OCc1ccc(O[C@@H]4O[C@H](C(=O)O)[C@@H](O)[C@H](O)[C@H]4O)cc1OCCOCCNC(=O)CCN1C(=O)C=CC1=O)(C3)C2] |
| 2.76 (con- trol) | WD | 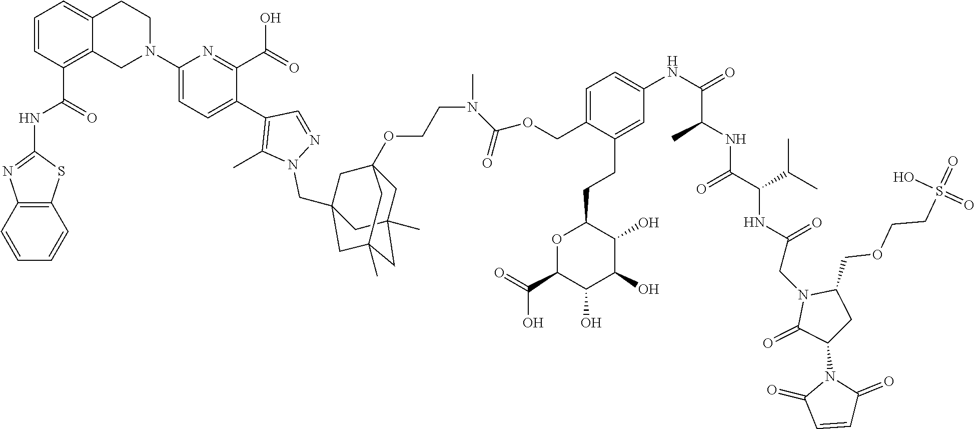 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(C)C(=O)OCc1ccc(NC(=O)[C@H](C)NC(=O)[C@@H](NC(=O)CN4C(=O)[C@@H](N5C(=O)C=CC5=O)C[C@H]4COCCS(=O)(=O)O)C(C)C)cc1CC[C@@H]1O[C@H](C(=O)O)[C@@H](O)[C@H](O)[C@H]1O)(C3)C2] |
| 2.77 (con- trol) | TV | 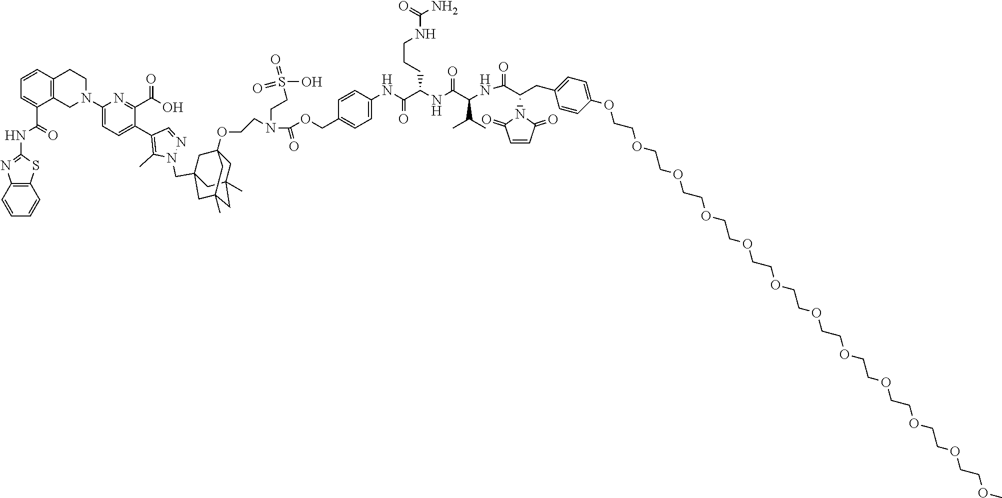 [SMILES: COCCOCCOCCOCCOCCOCCOCCOCCOCCOCCOCCOc1ccc(C[C@@H](C(=O)N[C@H](C(=O)N[C@@H](CCCNC(N)=O)C(=O)Nc2ccc(COC(=O)N(CCOC34CC5(C)CC(C)(CC(Cn6ncc(-c7ccc(N8CCc9cccc(C(=O)Nc%10nc%11ccccc%11s%10)c9C8)nc7C(=O)O)c6C)(C5)C3)C4)CCS(=O)(=O)O)cc2)C(C)C)N2C(=O)C=CC2=O)cc1] |
| 2.78 (con- trol) | YY | 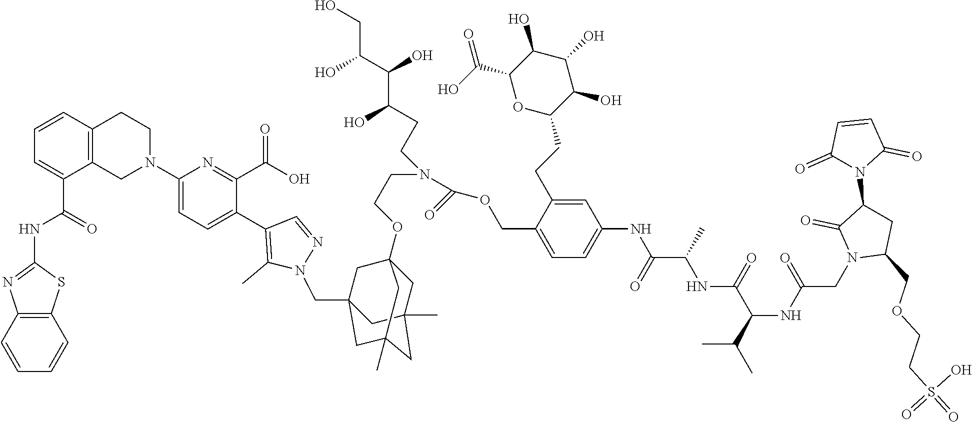 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CC[C@@H](O)[C@H](O)[C@H](O)CO)C(=O)OCc1ccc(NC(=O)[C@H](C)NC(=O)[C@@H](NC(=O)CN4C(=O)[C@@H](N5C(=O)C=CC5=O)C[C@H]4COCCS(=O)(=O)O)C(C)C)cc1CC[C@@H]1O[C@H](C(=O)O)[C@@H](O)[C@H](O)[C@H]1O)(C3)C2] |
| 2.79 (con- trol) | AAA | 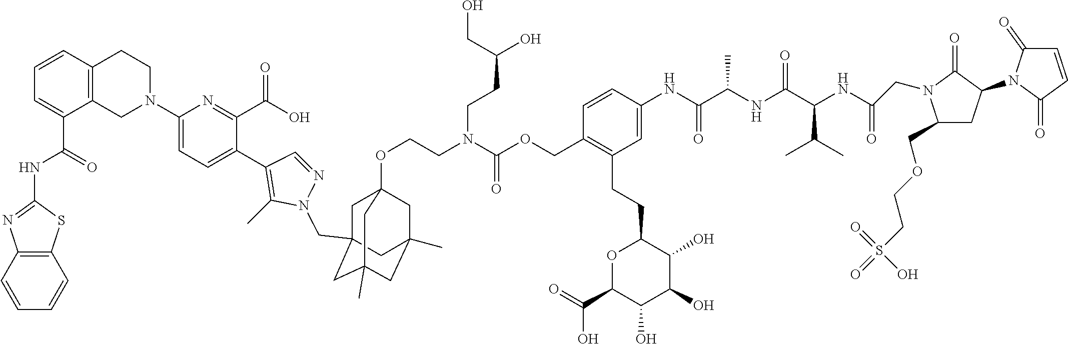 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CC[C@H](O)CO)C(=O)OCc1ccc(NC(=O)[C@H](C)NC(=O)[C@@H](NC(=O)CN4C(=O)[C@@H](N5C(=O)C=CC5=O)C[C@H]4COCCS(=O)(=O)O)C(C)C)cc1CC[C@@H]1O[C@H](C(=O)O)[C@@H](O)[C@H](O)[C@H]1O)(C3)C2] |
| 2.80 (con- trol) | AAD | 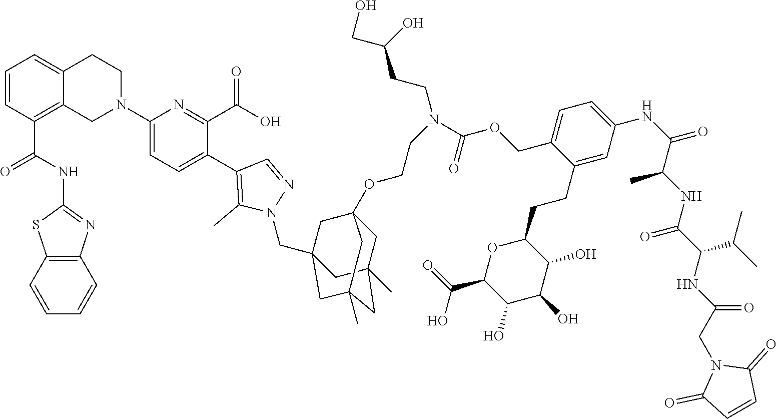 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CC[C@H](O)CO)C(=O)OCc1ccc(NC(=O)[C@H](C)NC(=O)[C@@H](NC(=O)CN4C(=O)C=CC4=O)C(C)C)cc1CC[C@@H]1O[C@H](C(=O)O)[C@@H](O)[C@H](O)[C@H]1O)(C3)C2] |

In certain embodiments, the synthon is selected from the group consisting of synthon examples 2.1, 2.2, 2.3, 2.4, 2.5, 2.6, 2.7, 2.8, 2.9, 2.10, 2.11, 2.12, 2.13, 2.14, 2.15, 2.16, 2.17, 2.18, 2.19, 2.20, 2.21, 2.22, 2.23, 2.24, 2.25, 2.26, 2.27, 2.28, 2.29, 2.30, 2.31, 2.34, 2.35, 2.36, 2.37, 2.38, 2.39, 2.40, 2.41, 2.42, 2.43, 2.44, 2.45, 2.46, 2.47, 2.48, 2.49, 2.50, 2.51, 2.52, 2.53, 2.54, 2.55, 2.56, 2.57, 2.58, 2.59, 2.60, 2.61, 2.62, 2.63, 2.64, 2.65, 2.66, 2.67, 2.68, 2.69, 2.70, 2.71, 2.72, and pharmaceutically acceptable salts thereof. The corresponding compound names of these synthons are provided below:
N-[6-(2,5-dioxo-2,5-dihydro-1H-pyrrol-1-yl)hexanoyl]-L-valyl-N-{4-[({[2-({3-[(4-{6-[l-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]phenyl}-N⁵-carbamoyl-L-ornithinamide;
N-[6-(2,5-dioxo-2,5-dihydro-1H-pyrrol-1-yl)hexanoyl]-L-valyl-N-{4-[({[2-({3-[(4-{6-[4-(1,3-benzothiazol-2-ylcarbamoyl)-3,4-dihydro-2H-1,4-benzoxazin-6-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]phenyl}-N⁵-carbamoyl-L-ornithinamide;
N-[6-(2,5-dioxo-2,5-dihydro-1H-pyrrol-1-yl)hexanoyl]-L-valyl-N-{4-[({[2-({3-[(4-{6-[4-(1,3-benzothiazol-2-ylcarbamoyl)-1-methyl-1,2,3,4-tetrahydroquinoxalin-6-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]phenyl}-N⁵-carbamoyl-L-ornithinamide;
4-[(1E)-3-({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl beta-D-glucopyranosiduronic acid;
4-[(1E)-3-({[2-({3-[(4-{6-[4-(1,3-benzothiazol-2-ylcarbamoyl)-1-methyl-1,2,3,4-tetrahydroquinoxalin-6-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl beta-D-glucopyranosiduronic acid;
4-[(1E)-3-({[2-({3-[(4-{6-[4-(1,3-benzothiazol-2-ylcarbamoyl)-3,4-dihydro-2H-1,4-benzoxazin-6-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl beta-D-glucopyranosiduronic acid;
4-[(1E)-3-({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl beta-D-glucopyranosiduronic acid;
4-[(1E)-3-({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.13.7]dec-1-yl}oxy)ethyl](oxetan-3-yl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl beta-D-glucopyranosiduronic acid;
4-[(1E)-3-({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl beta-D-glucopyranosiduronic acid;
4-[(1E)-3-({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)prop-1-en-1-yl]-2-({N-[(2,5-dioxo-2,5-dihydro-1H-pyrrol-1-yl)acetyl]-beta-alanyl}amino)phenyl beta-D-glucopyranosiduronic acid;
4-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-3-[2-(2-{[3-(2,5-dioxo-2,5-dihydro-1H-pyrrol-1-yl)propanoyl]amino} ethoxy)ethoxy]phenyl beta-D-glucopyranosiduronic acid;
6-[8-(1,3-benzothiazol-2-ylcarbamoyl)-5-methoxy-3,4-dihydroisoquinolin-2(1H)-yl]-3-(l-{[3-(2-{[({(2E)-3-[4-{[(2S,3R,4S,5S,6S)-6-carboxy-3,4,5-trihydroxytetrahydro-2H-pyran-2-yl]oxy}-3-({3-[({[(2E)-3-(4-{[(2S,3R,4S,5S,6S)-6-carboxy-3,4,5-trihydroxytetrahydro-2H-pyran-2-yl]oxy}-3-[(3-{[3-(2,5-dioxo-2,5-dihydro-1H-pyrrol-1-yl)propanoyl]amino}propanoyl)amino]phenyl)prop-2-en-1-yl]oxy} carbonyl)amino]propanoyl}amino)phenyl]prop-2-en-1-yl}oxy)carbonyl](2-methoxyethyl)amino}ethoxy)-5,7-dimethyltricyclo[3.3.1.1^{3,7}]dec-1-yl]methyl}-5-methyl-1H-pyrazol-4-yl)pyridine-2-carboxylic;
4-[({[2-(2-{2-[({[2-({3-[4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-5-(beta-D-glucopyranuronosyloxy)phenoxy}ethoxy)ethyl]carbamoyl}oxy)methyl]-3-[2-(2-{[3-(2,5-dioxo-2,5-dihydro-1H-pyrrol-1-yl)propanoyl]amino}ethoxy)ethoxy]phenyl beta-D-glucopyranosiduronic acid;
4-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-3-[2-(2-{[(2,5-dioxo-2,5-dihydro-1H-pyrrol-1-yl)acetyl]amino}ethoxy)ethoxy]phenyl beta-D-glucopyranosiduronic acid;
6-[1-(1,3-benzothiazol-2-ylcarbamoyl)-1,2,3,4-tetrahydroquinolin-7-yl]-3-{1-[(3-{[34-(2,5-dioxo-2,5-dihydro-1H-pyrrol-1-yl)-3-methyl-4,32-dioxo-7,10,13,16,19,22,25,28-octaoxa-3,31-diazatetratriacont-1-yl]oxy}-5,7-dimethyltricyclo[3.3.1.1^{3,7}]dec-1-yl)methyl]-5-methyl-1H-pyrazol-4-yl}pyridine-2-carboxylic acid;
4-[({[2-({3-[(4-{6-[8-(1,3-benzothiazol-2-ylcarbamoyl)-5-cyano-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]carbamoyl}oxy)methyl]-3-[2-(2-{[3-(2,5-dioxo-2,5-dihydro-1H-pyrrol-1-yl)propanoyl]amino}ethoxy)ethoxy]phenyl beta-D-glucopyranosiduronic acid;
4-[(1E)-3-({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)prop-1-en-1-yl]-2-({N-[3-(2,5-dioxo-2,5-dihydro-1H-pyrrol-1-yl)propanoyl]-beta-alanyl}amino)phenyl beta-D-glucopyranosiduronic acid;
N-[(2,5-dioxo-2,5-dihydro-1H-pyrrol-1-yl)acetyl]-3-sulfo-L-alanyl-N-{5-[(1E)-3-({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl] (2-methoxyethyl)carbamoyl}oxy)prop-1-en-1-yl]-2-(beta-D-glucopyranuronosyloxy)phenyl}-beta-alaninamide;
N-[3-(2,5-dioxo-2,5-dihydro-1H-pyrrol-1-yl)propanoyl]-3-sulfo-L-alanyl-N-{5-[(1E)-3-({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)prop-1-en-1-yl]-2-(beta-D-glucopyranuronosyloxy)phenyl}-beta-alaninamide;
N-[(2,5-dioxo-2,5-dihydro-1H-pyrrol-1-yl)acetyl]-beta-alanyl-N-{5-[(1E)-3-({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)prop-1-en-1-yl]-2-(beta-D-glucopyranuronosyloxy)phenyl}-beta-alaninamide;
N-[3-(2,5-dioxo-2,5-dihydro-1H-pyrrol-1-yl)propanoyl]-beta-alanyl-N-{5-[(1E)-3-({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)prop-1-en-1-yl]-2-(beta-D-glucopyranuronosyloxy)phenyl}-beta-alaninamide;
4-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-3-{2-[2-({N-[(2,5-dioxo-2,5-dihydro-1H-pyrrol-1-yl)acetyl]-3-sulfo-L-alanyl}amino)ethoxy]ethoxy}phenyl beta-D-glucopyranosiduronic acid;
4-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-3-{2-[2-({N-[3-(2,5-dioxo-2,5-dihydro-1H-pyrrol-1-yl)propanoyl]-3-sulfo-L-alanyl}amino)ethoxy]ethoxy}phenyl beta-D-glucopyranosiduronic acid;
4-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-3-{2-[2-({N-[(2,5-dioxo-2,5-dihydro-1H-pyrrol-1-yl)acetyl]-beta-alanyl}amino)ethoxy]ethoxy}phenyl beta-D-glucopyranosiduronic acid;
4-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-3-{2-[2-({N-[3-(2,5-dioxo-2,5-dihydro-1H-pyrrol-1-yl)propanoyl]-beta-alanyl}amino)ethoxy]ethoxy}phenyl beta-D-glucopyranosiduronic acid;
2-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-5-{2-[2-({N-[3-(2,5-dioxo-2,5-dihydro-1H-pyrrol-1-yl)propanoyl]-3-sulfo-L-alanyl}amino)ethoxy]ethoxy}phenyl beta-D-glucopyranosiduronic acid;
2-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-5-{2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid;
4-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-3-[3-({N-[6-(2,5-dioxo-2,5-dihydro-1H-pyrrol-1-yl)hexanoyl]-3-sulfo-L-alanyl}amino)propoxy]phenyl beta-D-glucopyranosiduronic acid;
4-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-[3-({N-[6-(2,5-dioxo-2,5-dihydro-1H-pyrrol-1-yl)hexanoyl]-3-sulfo-L-alanyl}amino)propoxy]phenyl beta-D-glucopyranosiduronic acid;
N-[6-(2,5-dioxo-2,5-dihydro-1H-pyrrol-1-yl)hexanoyl]-L-valyl-N-{4-[({[(3S)-1-{8-(1,3-benzothiazol-2-ylcarbamoyl)-2-[6-carboxy-5-(1-{[3-(2-methoxy ethoxy)-5,7-dimethyltricyclo[3.3.1.1^{3,7}]dec-1-yl]methyl}-5-methyl-1H-pyrazol-4-yl)pyridin-2-yl]-1, 2, 3, 4-tetrahydroisoquinolin-6-yl}pyrrolidin-3-yl]carbamoyl}oxy)methyl]phenyl}-L-alaninamide;
N-[6-(2,5-dioxo-2,5-dihydro-1H-pyrrol-1-yl)hexanoyl]-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfamoylethyl)carbamoyl}oxy)methyl]phenyl}-N⁵-carbamoyl-L-omithinamide;
4-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-3-{2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid;
2-[({[2-({3-[(4-{6-[5-(1,3-benzothiazol-2-ylcarbamoyl)quinolin-3-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-5-{2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid;
2-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-5-[2-(2-{[6-(2,5-dioxo-2,5-dihydro-1H-pyrrol-1-yl)hexanoy]amino}ethoxy)ethoxy]phenyl beta-D-glucopyranosiduronic acid;
4-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-{2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid;
2-[({[2-({3-[(4-{6-[4-(1,3-benzothiazol-2-ylcarbamoyl)quinolin-6-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-5-[2-(2-{[6-(2,5-dioxo-2,5-dihydro-1H-pyrrol-1-yl)hexanoyl]amino}ethoxy)ethoxy]phenyl beta-D-glucopyranosiduronic acid;
4-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-{2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid;
4-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-(3-{[6-(2,5-dioxo-2,5-dihydro-1H-pyrrol-1-yl)hexanoyl]amino}propoxy)phenyl beta-D-glucopyranosiduronic acid;
4-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-[3-({N-[6-(2,5-dioxo-2,5-dihydro-1H-pyrrol-1-yl)hexanoyl]-3-sulfo-L-alanyl}amino)propoxy]phenyl beta-D-glucopyranosiduronic acid;
2-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-5-{2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid;
4-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-3-{2-[2-({N-[6-(2,5-dioxo-2,5-dihydro-1H-pyrrol-1-yl)hexanoyl]-3-sulfo-L-alanyl}amino)ethoxy]ethoxy} phenyl beta-D-glucopyranosiduronic acid;
N-[6-(2,5-dioxo-2,5-dihydro-1H-pyrrol-1-yl)hexanoyl]-L-valyl-N-[4-({[{2-[{8-(1,3-benzothiazol-2-ylcarbamoyl)-2-[6-carboxy-5-(1-{[3-(2-methoxyethoxy)-5,7-dimethyltricyclo[3.3.1.1^{3,7}]dec-1-yl]methyl}-5-methyl-1H-pyrazol-4-yl)pyridin-2-yl]-1,2,3,4-tetrahydroisoqiunolin-6-yl}(methyl)amino]ethyl}(methyl)carbamoyl]oxy}methyl)phenyl]-L-alaninamide;
N-[6-(2,5-dioxo-2,5-dihydro-1H-pyrrol-1-yl)hexanoyl]-L-valyl-N-{4-[({[2-({3-[(4-{6-[8-(1,3-benzothiazol-2-ylcarbamoyl)-6-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]phenyl}-L-alaninamide;
2-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxyethyl)carbamoyl}oxy)methyl]-5-{2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid;
2-[({[2-({3-[(4-{6-[5-(1,3-benzothiazol-2-ylcarbamoyl)quinolin-3-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-5-[2-(2-{[6-(2,5-dioxo-2,5-dihydro-1H-pyrrol-1-yl)hexanoyl]amino}ethoxy)ethoxy]phenyl beta-D-glucopyranosiduronic acid;
4-[({[2-({3-[(4-{6-[5-(1,3-benzothiazol-2-ylcarbamoyl)quinolin-3-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-[2-(2-{[6-(2,5-dioxo-2,5-dihydro-1H-pyrrol-1-yl)hexanoyl]amino}ethoxy)ethoxy]phenyl beta-D-glucopyranosiduronic acid;
6-[5-(1,3-benzothiazol-2-ylcarbamoyl)quinolin-3-yl]-3-(1-{[3-(2-{[6-(2,5-dioxo-2,5-dihydro-1H-pyrrol-1-yl)hexanoyl](methyl)amino}ethoxy)-5,7-dimethyltricyclo[3.3.1.1^{3,7}]dec-1-yl]methyl}-5-methyl-1H-pyrazol-4-yl)pyridine-2-carboxylic acid;
4-[({[2-({3-[(4-{6-[7-(1,3-benzothiazol-2-ylcarbamoyl)-1H-indol-2-yl]-2-carboxypyridin-3-yl}-5-methyl-1H-pyrazol-1-yl)methyl]-5,7-dimethyltricyclo[3.3.1.1¹⁷]dec-1-yl}oxy)ethyl](methyl)carbamoyl}oxy)methyl]-2-({N-[3-(2,5-dioxo-2,5-dihydro-1H-pyrrol-1-yl)propanoyl]-beta-alanyl}amino)phenyl beta-D-glucopyranosiduronic acid;
4-[({[2-({3-[(4-{6-[7-(1,3-benzothiazol-2-ylcarbamoyl)-1H-indol-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-[2-(2-{[3-(2,5-dioxo-2,5-dihydro-1H-pyrrol-1-yl)propanoyl]amino}ethoxy)ethoxy]phenyl beta-D-glucopyranosiduronic acid;
4-[({[2-({3-[(4-{6-[7-(1,3-benzothiazol-2-ylcarbamoyl)-1H-indol-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-{2-[2-({N-[3-(2,5-dioxo-2,5-dihydro-1H-pyrrol-1-yl)propanoyl]-3-sulfo-L-alanyl}amino)ethoxy]ethoxy}phenyl beta-D-glucopyranosiduronic acid;
4-[({[2-({3-[(4-{6-[7-(1,3-benzothiazol-2-ylcarbamoyl)-3-methyl-1H-indol-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-[2-(2-{[3-(2,5-dioxo-2,5-dihydro-1H-pyrrol-1-yl)propanoyl]amino}ethoxy)ethoxy)]phenyl beta-D-glucopyranosiduronic acid;
N-[6-(2,5-dioxo-2,5-dihydro-1H-pyrrol-1-yl)hexanoyl]-L-valyl-N-{4-[({[2-({3-[(4-{6-[4-(1,3-benzothiazol-2-ylcarbamoyl)isoquinolin-6-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]phenyl}-N⁵-carbamoyl-L-omithinamide;
4-[({[2-({3-[(4-{6-[1-(1,3-benzothiazol-2-ylcarbamoyl)-5,6-dihydroimidazo[1,5-a]pyrazin-7(8H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]carbamoyl}oxy)methyl]-3-[2-(2-{[(2,5-dioxo-2,5-dihydro-1H-pyrrol-1-yl)acetyl]amino}ethoxy)ethoxy]phenyl beta-D-glucopyranosiduronic acid;
2-[({[2-({3-[(4-{6-[5-(1,3-benzothiazol-2-ylcarbamoyl)quinolin-3-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]carbamoyl}oxy)methyl]-4-[19-(2,5-dioxo-2,5-dihydro-1H-pyrrol-1-yl)-14-oxo-4,7,10-trioxa-13-azanonadec-1-yl]phenyl beta-D-glucopyranosiduronic acid;
4-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-3-[4-({N-[6-(2,5-dioxo-2,5-dihydro-1H-pyrrol-1-yl)hexanoyl]-3-sulfo-L-alanyl}amino)butyl]phenyl beta-D-glucopyranosiduronic acid;
2-{6-[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]-2-methyl-3,3-dioxido-7-oxo-8-oxa-3lambda⁶-thia-2,6-diazanonan-9-yl}-5-(4-{[(2,5-dioxo-2,5-dihydro-1H-pyrrol-1-yl)acetyl]amino}butyl)phenyl beta-D-glucopyranosiduronic acid;
6-[8-(1,3-benzothiazol-2-ylcarbamoyl)-3,4-dihydroisoquinolin-2(1H)-yl]-3-(1-{[3-(2-{({[2-{[(2S,3R,4S,5S,6S)-6-carboxy-3,4,5-trihydroxytetrahydro-2H-pyran-2-yl]oxy}-4-(4-{[(2,5-dioxo-2,5-dihydro-1H-pyrrol-1-yl)acetyl]amino}butyl)benzyl]oxy}carbonyl)[3-(dimethylamino)-3-oxopropyl]amino}ethoxy)-5,7-dimethyltricyclo[3.3.1.1^{3,7}]dec-1-yl]methyl}-5-methyl-1H-pyrazol-4-yl)pyridine-2-carboxylic acid;
2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfamoylethyl)carbamoyl}oxy)methyl]-5-(4-{[(2,5-dioxo-2,5-dihydro-1H-pyrrol-1-yl)acetyl]amino}butyl)phenyl beta-D-glucopyranosiduronic acid;
6-[8-(1,3-benzothiazol-2-ylcarbamoyl)-3,4-dihydroisoquinolin-2(1H)-yl]-3-(1-{[3-(2-{({[2-{[(2S,3R,4S,5S,6S)-6-carboxy-3,4,5-trihydroxytetrahydro-2H-pyran-2-yl]oxy}-4-(4-{[(2,5-dioxo-2,5-dihydro-1H-pyrrol-1-yl)acetyl]amino}butyl)benzyl]oxy}carbonyl)[3-(methylamino)-3-oxopropyl]amino}ethoxy)-5,7-dimethyltricyclo[3.3.1.1^{3,7}]dec-1-yl]methyl}-5-methyl-1H-pyrazol-4-yl)pyridine-2-carboxylic acid;
3-{1-[(3-{2-[(3-amino-3-oxopropyl)({[2-{[(2S,3R,4S,5S,6S)-6-carboxy-3,4,5-trihydroxytetrahydro-2H-pyran-2-yl]oxy}-4-(4-{[(2,5-dioxo-2,5-dihydro-1H-pyrrol-1-yl)acetyl]amino}butyl)benzyl]oxy}carbonyl)amino]ethoxy}-5,7-dimethyltricyclo[3.3.1.1^{3,7}]dec-1-yl)methyl]-5-methyl-1H-pyrazol-4-yl}-6-[8-(1,3-benzothiazol-2-ylcarbamoyl)-3,4-dihydroisoquinolin-2(1H)-yl]pyridine-2-carboxylic acid;
2-[({[2-({3-[(4-{6-[3-(1,3-benzothiazol-2-ylcarbamoyl)-1H-indol-5-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-5-(4-{[(2,5-dioxo-2,5-dihydro-1H-pyrrol-1-yl)acetyl]amino}butyl)phenyl beta-D-glucopyranosiduronic acid;
2-[({[2-({3-[(4-{6-[1-(1,3-benzothiazol-2-ylcarbamoyl)-5,6-dihydroimidazo[1,5-a]pyrazin-7(8H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]carbamoyl}oxy)methyl]-5-(4-{[(2,5-dioxo-2,5-dihydro-1H-pyrrol-1-yl)acetyl]amino}butyl)phenyl beta-D-glucopyranosiduronic acid;
(6S)-2,6-anhydro-6-(2-{2-[({[2-({3-[(4-{6-[1-(1,3-benzothiazol-2-ylcarbamoyl)-5,6-dihydroimidazo[1,5-a]pyrazin-7(8H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]carbamoyl}oxy)methyl]-5-({N-[(2,5-dioxo-2,5-dihydro-1H-pyrrol-1-yl)acetyl]-L-valyl-L-alanyl}amino)phenyl}ethyl)-L-gulonic acid;
(6S)-2,6-anhydro-6-[2-(2-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-methoxy ethyl)carbamoyl}oxy)methyl]-5-{[N-({(3S,5S)-3-(2,5-dioxo-2,5-dihydro-1H-pyrrol-1-yl)-2-oxo-5-[(2-sulfoethoxy)methyl]pyrrolidin-1-yl}acetyl)-L-valyl-L-alanyl]amino}phenyl)ethyl]-L-gulonic acid;
8-[2-({[(3-amino-3-oxopropyl) {2-[(3-{[4-(6-{8-[(1,3-benzothiazol-2-yl)carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.1^{3,7}]decan-1-yl)oxy]ethyl}carbamoyl]oxy}methyl)-5-{[(2S)-2-({(2S)-2-[2-(2,5-dioxo-2,5-dihydro-1H-pyrrol-1-yl)acetamido]-3-methylbutanoyl}amino)propanoyl]amino}phenyl]-2,6-anhydro-7,8-dideoxy-L-glycero-L-gulo-octonic acid;
4-{[({2-[(3-{[4-(6-{8-[(1,3-benzothiazol)-2-yl)carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.1^{3,7}]decan-1-yl)oxy]ethyl}[3-(methylamino)-3-oxopropyl]carbamoyl)oxy]methyl}-3-{3-[2-(2,5-dioxo-2,5-dihydro-1H-pyrrol-1-yl)acetamido]propoxy}phenyl beta-D-glucopyranosiduronic acid;
2,6-anhydro-8-(2-{[({2-[(3-{[4-(6-{8-[(1,3-benzothiazol-2-yl)carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.1^{3,7}]decan-1-yl)oxy]ethyl}[3-(methylamino)-3-oxopropyl]carbamoyl)oxy]methyl}-5-{[(2S)-2-({(2S)-2-[2-(2,5-dioxo-2,5-dihydro-1H-pyrrol-1-yl)acetamido]-3-methylbutanoyl}amino)propanoyl]amino}phenyl)-7,8-dideoxy-L-glycero-L-gulo-octonic acid;
2,6-anhydro-8-(2-{[({2-[(3-{[4-(6-{8-[(1,3-benzothiazol-2-yl)carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.1^{3,7}]decan-1-yl)oxy]ethyl}[3-(methylamino)-3-oxopropyl]carbamoyl)oxy]methyl}-5-{[(2S)-2-{[(2S)-2-(2-{(3S,5S)-3-(2,5-dioxo-2,5-dihydro-1H-pyrrol-1-yl)-2-oxo-5-[(2-sulfoethoxy)methyl]pyrrolidin-1-yl}acetamido-3-methylbutanoyl]amino}propanoyl]amino}phenyl)-7,8-dideoxy-L-glycero-L-gulo-octonic acid;
6-{8-[(1,3-benzothiazol-2-yl)carbamoyl]-3,4-dihydroisoquinolin-2(1H)-yl}-3-[1-({3-[2-({[(4-{[(2S)-5-(carbamoylamino)-2-{[(2S)-2-{[6-(2,5-dioxo-2,5-dihydro-1H-pyrrol-1-yl)hexanoyl]amino}-3-methylbutanoyl]amino}pentanoyl]amino}phenyl)methoxy]carbonyl}amino)acetamido]-5,7-dimethyltricyclo[3.3.1.1^{3,7}]decan-1-yl}methyl)-5-methyl-1H-pyrazol-4-yl]pyridine-2-carboxylic acid; and
8-[2-({[(3-amino-3-oxopropyl){2-[(3-{[4-(6-{8-[(1,3-benzothiazol-2-yl)carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.1^{3,7}]decan-1-yl)oxy]ethyl}carbamoyl]oxy}methyl)-5-{[(2S)-2-{[(2S)-2-(2-{(3S,5S)-3-(2,5-dioxo-2,5-dihydro-1H-pyrrol-1-yl)-2-oxo-5-[(2-sulfoethoxy)methyl]pyrrolidin-1-yl}acetamido)-3-methylbutanoyl]amino}propanoyl]amino}phenyl]-2,6-anhydro-7,8-dideoxy-L-glycero-L-gulo-octonic acid.

In certain embodiments, the ADC, or a pharmaceutically acceptable salt thereof, comprises

D is the Bcl-xL inhibitor selected from the group consisting of the following compounds modified in that the hydrogen corresponding to the # position is not present, forming a monoradical:

W3.01, W3.02, W3.03, W3.04, W3.05, W3.06, W3.07, W3.08, W3.09, W3.10, W3.11, W3.12, W3.13, W3.14, W3.15, W3.16, W3.17, W3.18, W3.19, W3.20, W3.21, W3.22, W3.23, W3.24, W3.25, W3.26, W3.27, W3.28, W3.29, W3.30, W3.31, W3.32, W3.33, W3.34, W3.35, W3.36, W3.37, W3.38, W3.39, W3.40, W3.41, W3.42, and W3.43 and pharmaceutically acceptable salts thereof;

L is selected from the group consisting of linkers IVa.1-IVa.8, IVb.1-IVb.19, IVc.1-IVc.7. IVd.1-IVd.4, Va.1-Va.12, Vb.1-Vb.10, Vc.1-Vc.11, Vd.1-Vd.6, Ve.1-Ve.2, VIa.1, VIc.1-Vlc.2, VId.1-VId.4, VIIa.1-VIIa.4, VIIb.1-VIIb.8, and VIIc.1-VIIc.6, wherein each linker has reacted with the antibody, Ab, forming a covalent attachment;

LK is thioether, and

m is an integer ranging from 1 to 8.

In certain embodiments, the ADC, or a pharmaceutically acceptable salt thereof,

D is the Bcl-xL inhibitor selected from the group consisting of the following compounds modified in that the hydrogen corresponding to the # position is not present, forming a monoradical:
3-(1-{[3-(2-aminoethoxy)-5,7-dimethyltricyclo[3.3.1.1^{3,7}]dec-1-yl]methyl}-5-methyl-1H-pyrazol-4-yl)-6-[1-(1,3-benzothiazol-2-ylcarbamoyl)-5,6-dihydroimidazo[1,5-a]pyrazin-7(8H)-yl]pyridine-2-carboxylic acid;
6-[8-(1,3-benzothiazol-2-ylcarbamoyl)naphthalen-2-yl]-3-[1-({3,5-dimethyl-7-[2-(methylamino)ethoxy]tricyclo[3.3.1.1¹⁷]dec-1-yl}methyl)-5-methyl-1H-pyrazol-4-yl]pyridine-2-carboxylic acid;
6-[8-(1,3-benzothiazol-2-ylcarbamoyl)-5-methoxy-3,4-dihydroisoquinolin-2(1H)-yl]-3-{1-[(3-{2-[(2-methoxyethyl)amino]ethoxy}-5,7-dimethyltricyclo[3.3.1.1^{3,7}]dec-1-yl)methyl]-5-methyl-1H-pyrazol-4-yl} pyridine-2-carboxylic acid;
3-(1-{[3-(2-aminoethoxy)-5,7-dimethyltricyclo[3.3.1.1^{3,7}]dec-1-yl]methyl}-5-methyl-1H-pyrazol-4-yl)-6-[8-(1,3-benzothiazol-2-ylcarbamoyl)-5-cyano-3,4-dihydroisoquinolin-2(1H)-yl]pyridine-2-carboxylic acid;
6-[4-(1,3-benzothiazol-2-ylcarbamoyl)isoquinolin-6-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid;
3-{1-[(3-{2-[(3-amino-3-oxopropyl)amino]ethoxy}-5,7-dimethyltricyclo[3.3.1.1^{3,7}]dec-1-yl)methyl]-5-methyl-1H-pyrazol-4-yl}-6-[8-(1,3-benzothiazol-2-ylcarbamoyl)-3,4-dihydroisoquinolin-2(1H)-yl]pyridine-2-carboxylic acid;

and pharmaceutically acceptable salts thereof;

L is selected from the group consisting of linkers IVb.2, IVc.5, IVc.6, IVc.7, Vc.11. IVd.4, Vb.9, Vc.11, VIIa.1, VIIa.3. VIIc.1, VIIc.4, and VIIc.5 in either closed or open forms, and pharmaceutically acceptable salts thereof;

LK is thioether; and

m is an integer ranging from 2 to 4.

To form an ADC, the maleimide ring of a synthon (for example, the synthons listed in Table 5) may react with an antibody Ab, forming a covalent attachment as either a succinimide (closed form) or succinamide (open form). Similarly, other functional groups, e.g., acetyl halide or vinyl sulfone may react with an antibody, Ab, forming a covalent attachment.

In certain embodiments, the ADC, or a pharmaceutically acceptable salt thereof, is selected from the group consisting of huAb102-ZT, huAb102-ZZ, huAb102-XW, huAb102-SE, huAb3102-SR, huAb102-YG, huAb102-KZ, huAb104-ZT, huAb104-ZZ, huAb104-XW, huAb104-SE, huAb104-SR, huAb104-YG, huAb104-KZ, huAb108-ZT, huAb108-ZZ, huAb108-XW, huAb108-SE, huAb108-SR, huAb108-YG, huAb108-KZ, huAb110-ZT, hu110-ZZ, huAb110-XW, huAb110-SE, huAb3110-SR, huAb110-YG, and huAb110-KZ, wherein ZT, ZZ. XW, SE, SR, and YG are synthons disclosed in Table 5, and where in the synthons are either in open or closed form.

In certain embodiments, the ADC, or a pharmaceutically acceptable salt thereof, is

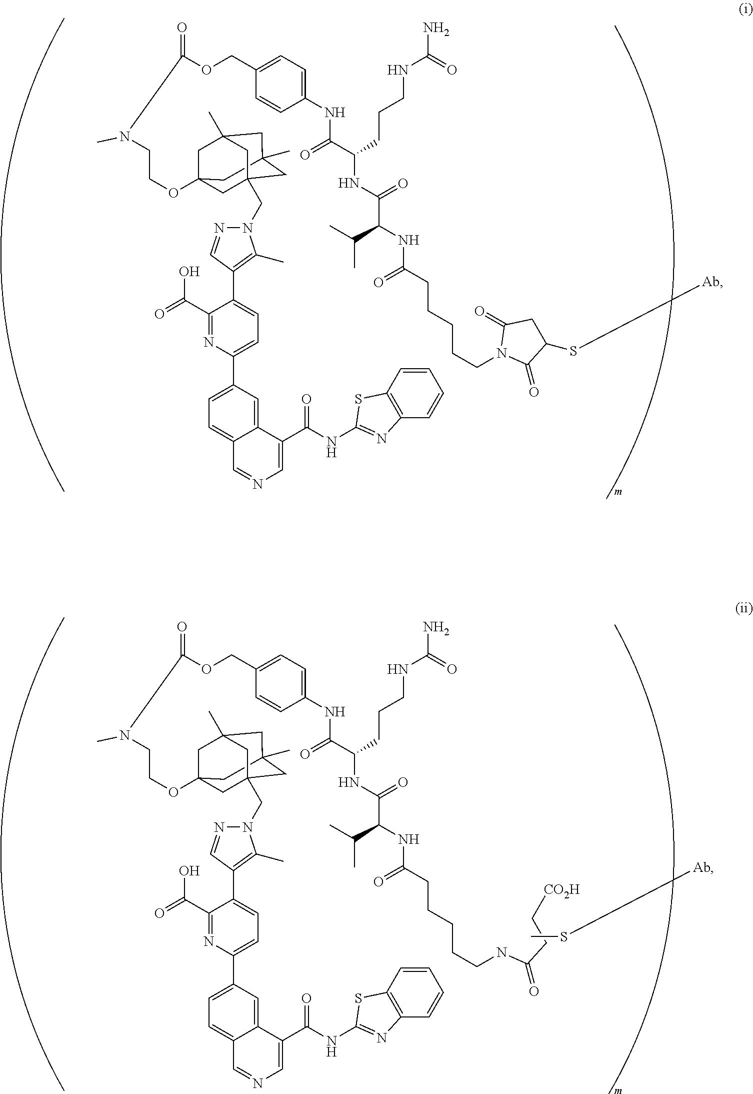

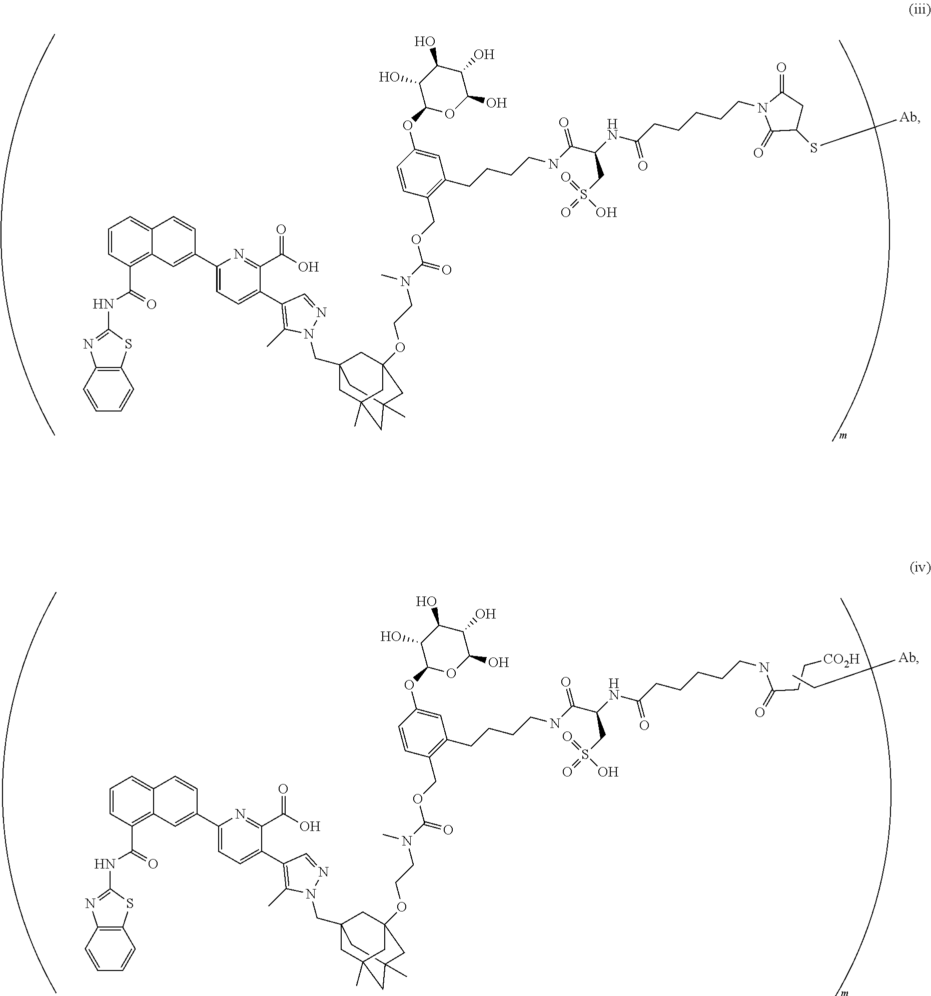

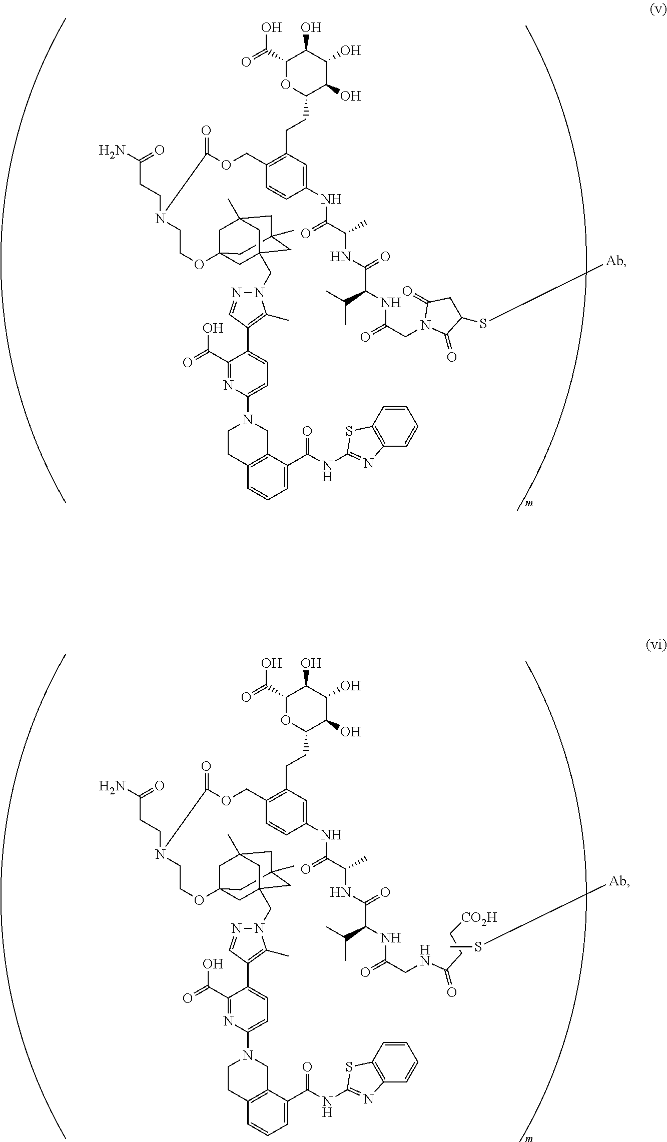

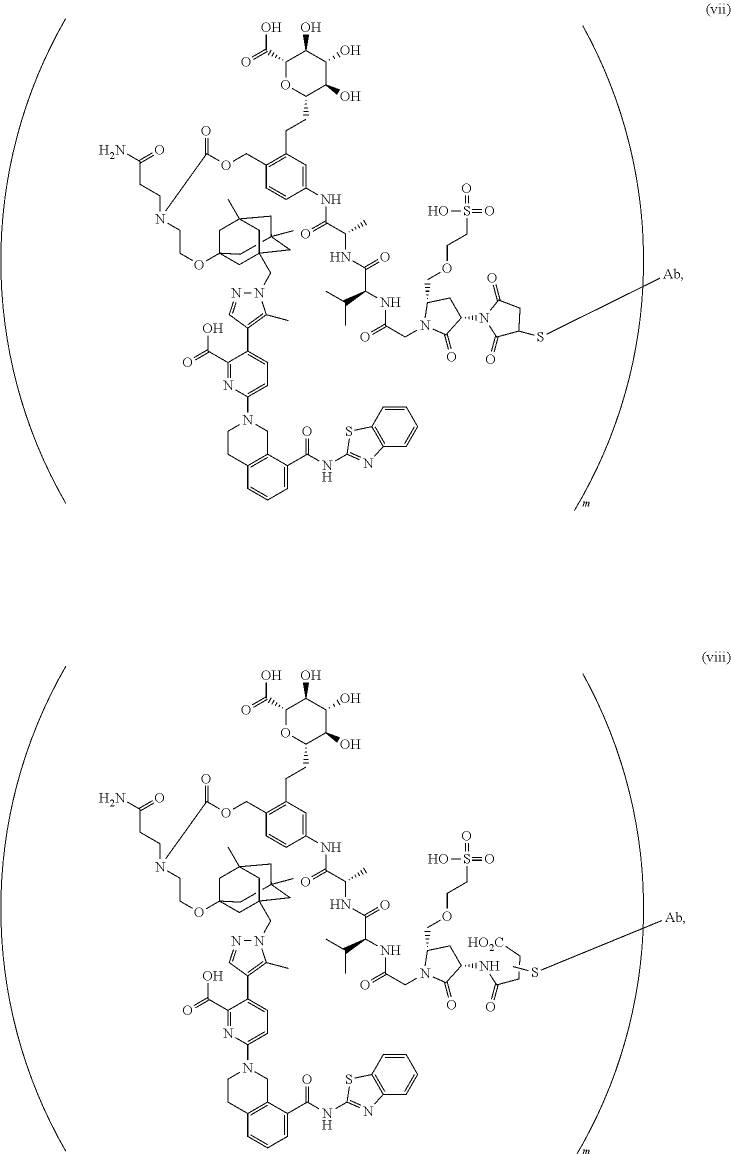

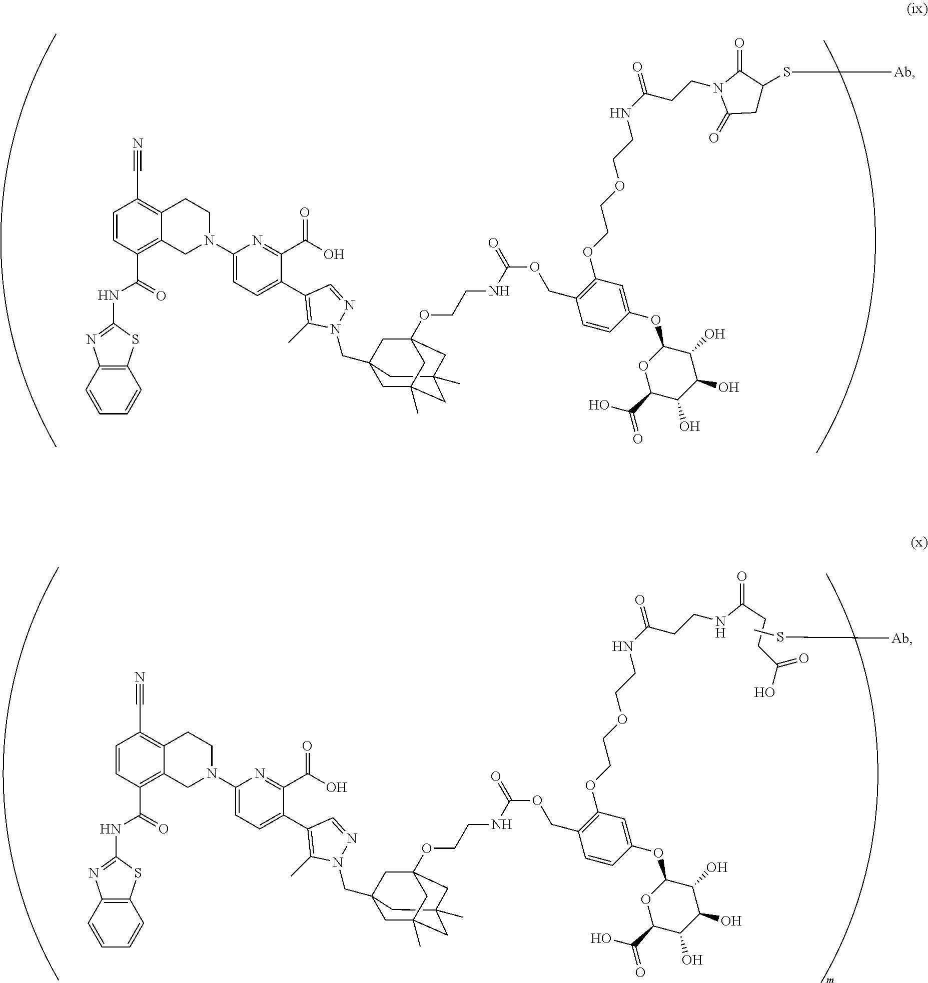

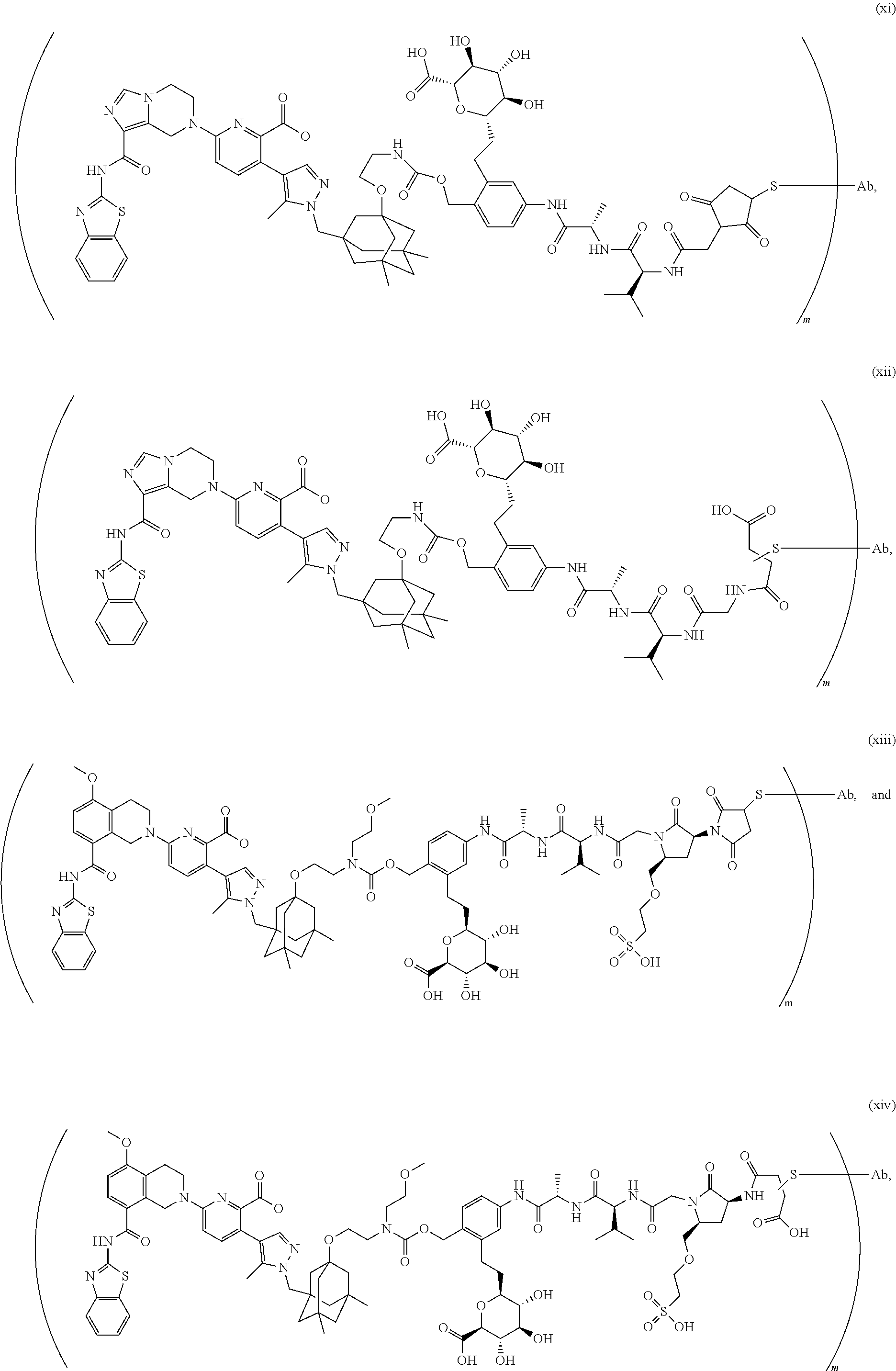

wherein m is an integer from 1 to 6. In a specific embodiment, m is an integer from 2 to 6.

In one embodiment, the ADC, or a pharmaceutically acceptable salt thereof, is

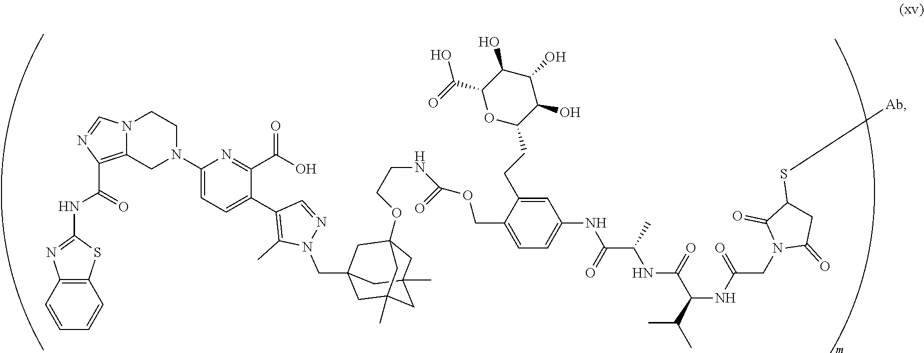

wherein m is 2, Ab is the anti-hCD98 antibody, wherein the anti-hCD98 antibody comprises the heavy and light chain CDRs of huAb102.

In one embodiment, the ADC, or a pharmaceutically acceptable salt thereof, is

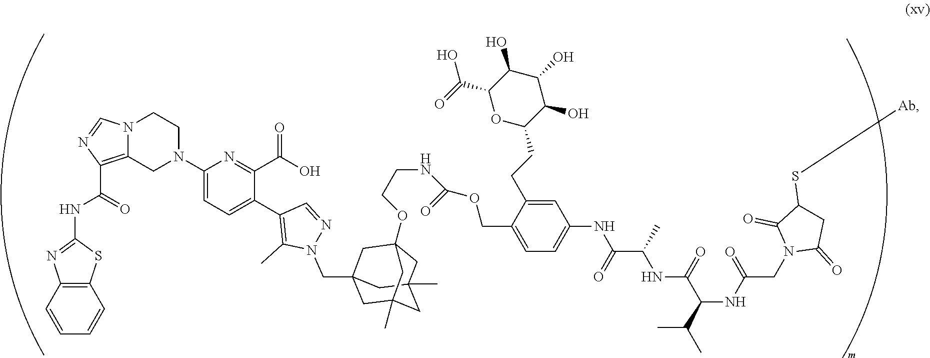

wherein m is 2, Ab is the anti-hCD98 antibody, wherein the anti-hCD98 antibody comprises the heavy and light chain CDRs of huAb104.

In one embodiment, the ADC, or a pharmaceutically acceptable salt thereof, is

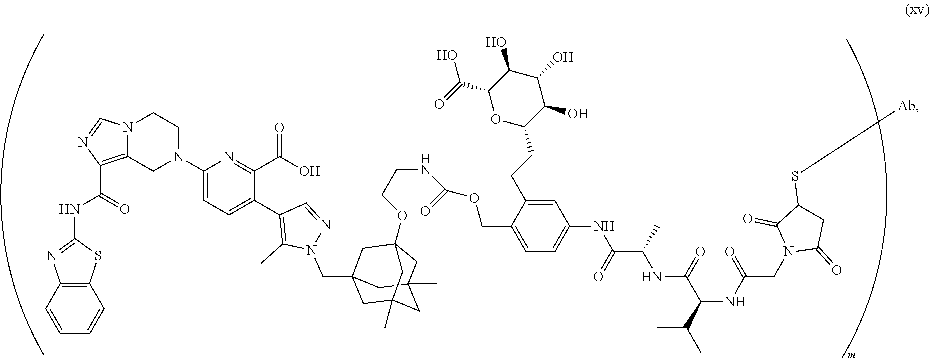

wherein m is 2, Ab is the anti-hCD98 antibody, wherein the anti-hCD98 antibody comprises the heavy and light chain CDRs of huAb104.

In one embodiment, the ADC, or a pharmaceutically acceptable salt thereof, is

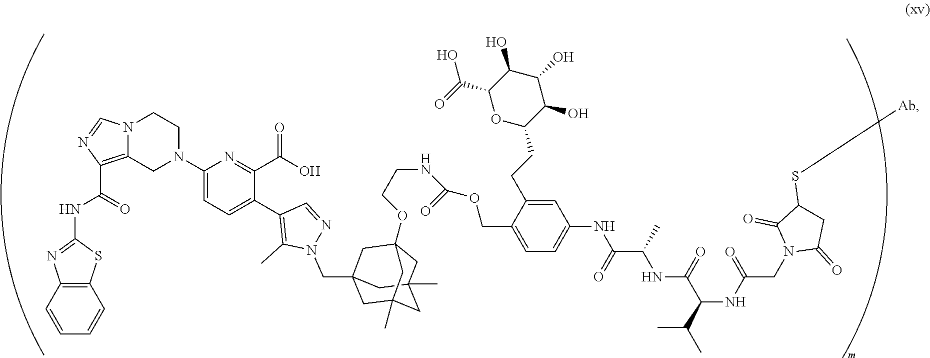

wherein m is 2, Ab is the anti-hCD98 antibody, wherein the anti-hCD98 antibody comprises the heavy and light chain CDRs of huAb110.

In one embodiment, the ADC, or a pharmaceutically acceptable salt thereof, is

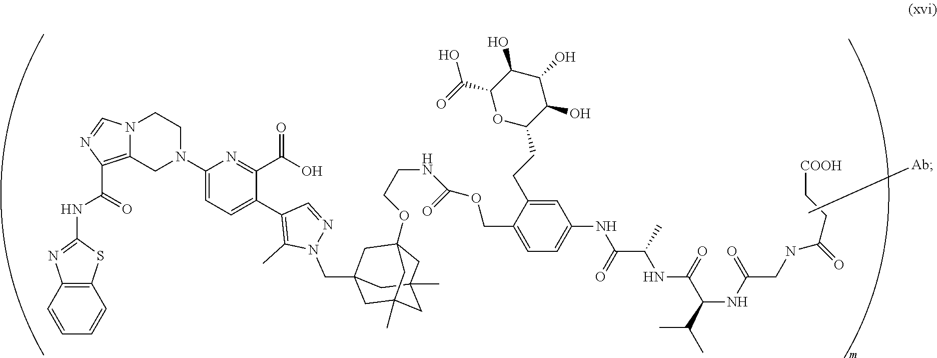

wherein m is 2, Ab is the anti-hCD98 antibody, wherein the anti-hCD98 antibody comprises the heavy and light chain CDRs of huAb102.

In one embodiment, the ADC, or a pharmaceutically acceptable salt thereof, is

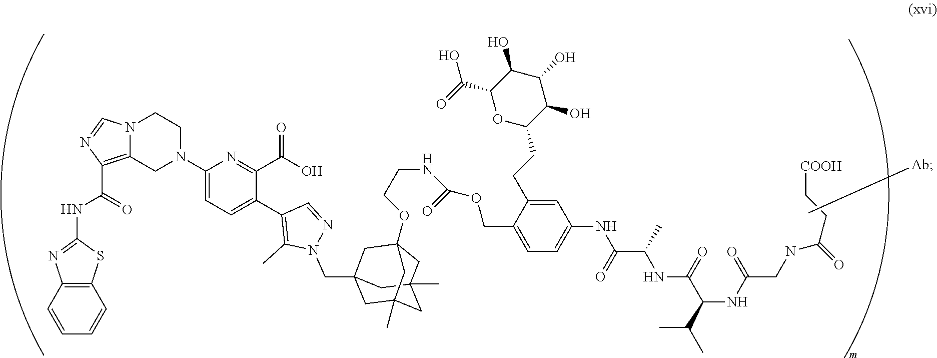

wherein m is 2, Ab is the anti-hCD98 antibody, wherein the anti-hCD98 antibody comprises the heavy and light chain CDRs of huAb104.

In one embodiment, the ADC, or a pharmaceutically acceptable salt thereof, is

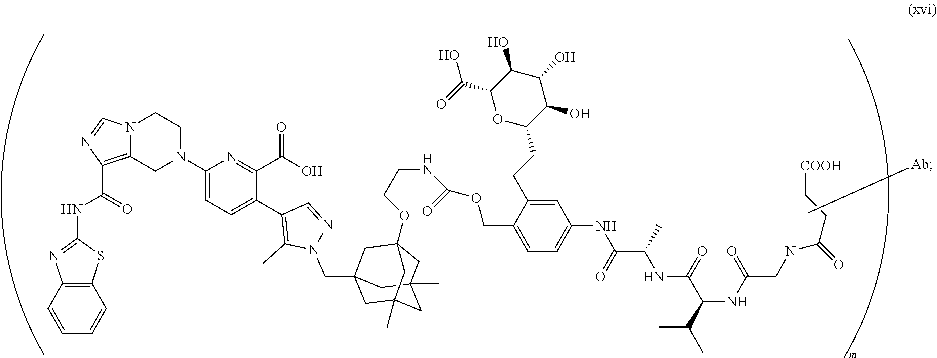

wherein m is 2, Ab is the anti-hCD98 antibody, wherein the anti-hCD98 antibody comprises the heavy and light chain CDRs of huAb108.

In one embodiment, the ADC, or a pharmaceutically acceptable salt thereof, is

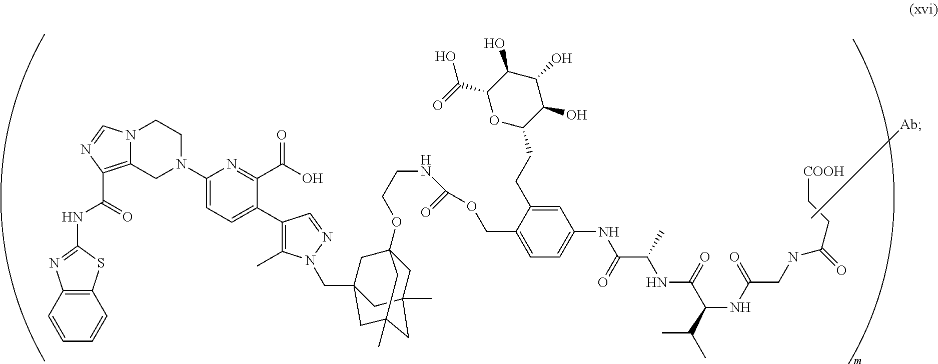

wherein m is 2, Ab is the anti-hCD98 antibody, wherein the anti-hCD98 antibody comprises the heavy and light chain CDRs of huAb110.

### IIIA.4. Methods of Synthesis of Bcl-xL ADCs

The Bcl-xL inhibitors and synthons described herein may be synthesized using standard, known techniques of organic chemistry. General schemes for synthesizing Bcl-xL inhibitors and synthons that may be used as-is or modified to synthesize the full scope of Bcl-xL inhibitors and synthons described herein are provided below. Specific methods for synthesizing exemplary Bcl-xL inhibitors and synthons that may be useful for guidance are provided in the Examples section.

ADCs may likewise be prepared by standard methods, such as methods analogous to those described in Hamblett et al., 2004. “Effects of Drug Loading on the Antitumor Activity of a Monoclonal Antibody Drug Conjugate”, Clin Cancer Res. 10:7063-7070; Doronina et al., 2003, “Development of potent and highly efficacious monoclonal antibody auristatin conjugates for cancer therapy.” Sat. Biotechnol. 21 (7): 778-784; and Francisco et al., 2003. “cAClO-vcMMAE, an anti-CD30-monomethylauristatin E conjugate with potent and selective antitumor activity.” Blood 102:1458-1465. For example, ADC's with four drugs per antibody may be prepared by partial reduction of the antibody with an excess of a reducing reagent such as DTT or TCEP at 37° C. for 30 min, then the buffer exchanged by elution through SEPHADEX® G-25 resin with 1 nM DTPA in DPBS. The eluent is diluted with further DPBS, and the thiol concentration of the antibody may be measured using 5,5′-dithiobis(2-nitrobenzoic acid) [Ellman's reagent]. An excess, for example 5-fold, of a linker-drug synthon is added at 4° C. for 1 hour, and the conjugation reaction may be quenched by addition of a substantial excess, for example 20-fold, of cysteine. The resulting ADC mixture may be purified on SEPHADEX G-25 equilibrated in PBS to remove unreacted synthons, desalted if desired, and purified by size-exclusion chromatography. The resulting ADC may then be then sterile-filtered, for example, through a 0.2 μm filter, and lyophilized if desired for storage. In certain embodiments, ail of the interchain cysteine disulfide bonds are replaced by linker-drug conjugates. One embodiment pertains to a method of making an ADC, comprising contacting a synthon described herein with an antibody under conditions in which the synthon covalently links to the antibody.

Specific methods for synthesizing exemplary ADCs that may be used to synthesize the full range of ADCs described herein are provided in the Examples section.

### IIIA.5. General Methods for Synthesizing Bcl-xL Inhibitors

In the schemes below, the various substituents Ar¹, Ar², Z¹, R⁴, R¹⁰, R^{11a} and R^{11b} are as defined in the Detailed Description section.

5.1.1 Synthesis of Compound (9)

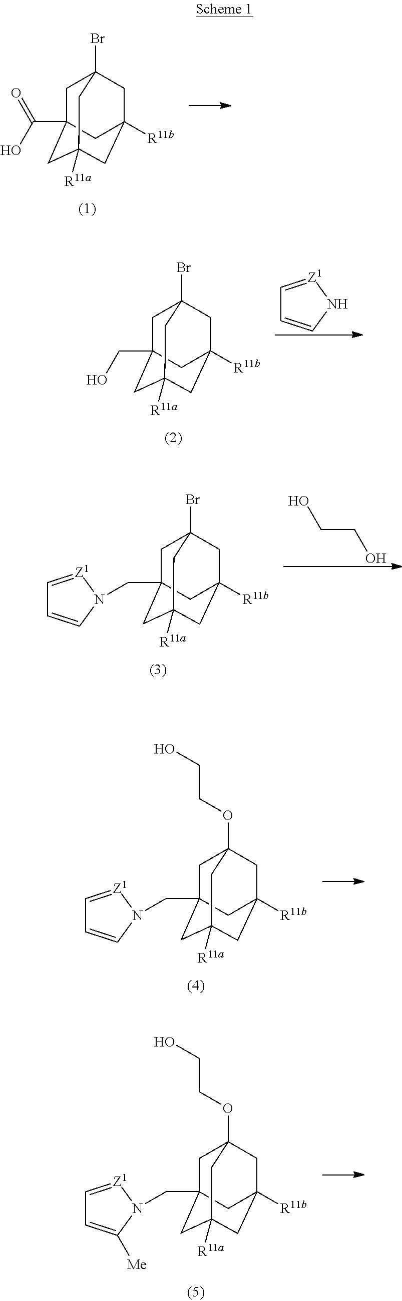

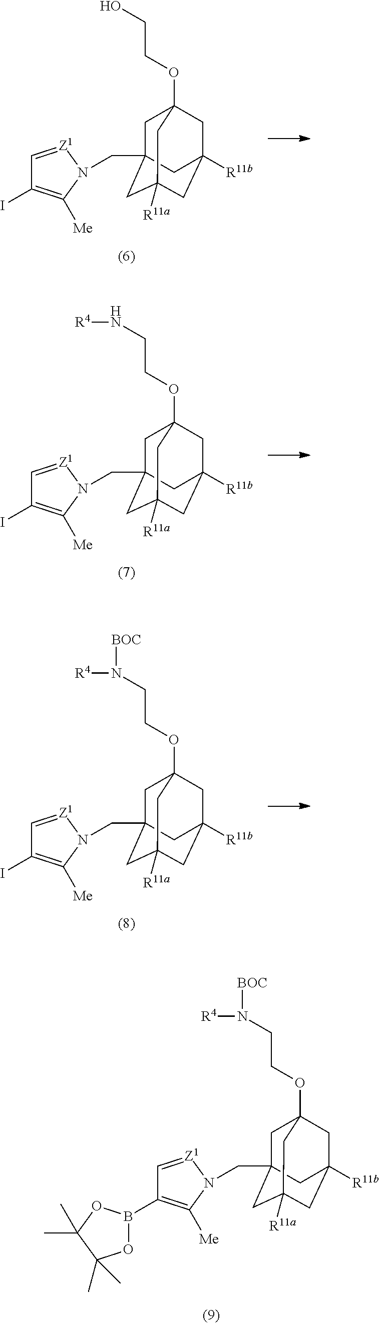

The synthesis of compound (9) is described in Scheme 1. Compound (1) can be treated with BH₃.THF to afford compound (2). The reaction is typically performed at ambient temperature in a solvent, such as, but not limited to, tetrahydrofuran. Compound (3) can be prepared by treating compound (2) with

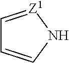

in the presence of cyanomethylenetributylphosphorane. The reaction is typically performed at an elevated temperature in a solvent such as, but not limited to, toluene. Compound (3) can be treated with ethane-1,2-diol in the presence of a base such as, but not limited to, triethylamine, to provide compound (4). The reaction is typically performed at an elevated temperature, and the reaction may be performed under microwave conditions. Compound (4) can be treated with a strong base, such as, but not limited to, n-butyllithium, followed by the addition of iodomethane, to provide compound (5). The addition and reaction is typically performed in a solvent such as, but not limited to, tetrahydrofuran, at a reduced temperature before warming up to ambient temperature for work up. Compound (5) can be treated with N-iodosuccinimide to provide compound (6). The reaction is typically performed at ambient temperature is a solvent such as, but not limited to, N,N-dimethylformamide. Compound (7) can be prepared by reacting compound (6) with methanesulfonyl chloride, in the presence of a base such as, but not limited to, triethylamine, followed by the addition of NHR⁴. The reaction with methanesulfonyl chloride is typically performed at low temperature, before increasing the temperature for the reaction with NHR⁴, and the reaction is typically performed in a solvent such as, but not limited to tetrahydrofuran. Compound (7) can be reacted with di-tert-butyl dicarbonate in the presence of 4-dimethylaminopyridine to provide compound (8). The reaction is typically performed at ambient temperature in a solvent such as, but not limited to tetrahydrofuran. The borylation of compound (8) to provide compound (9) can be performed under conditions described herein and readily available in the literature.

5.1.2, Synthesis of Compound (12)

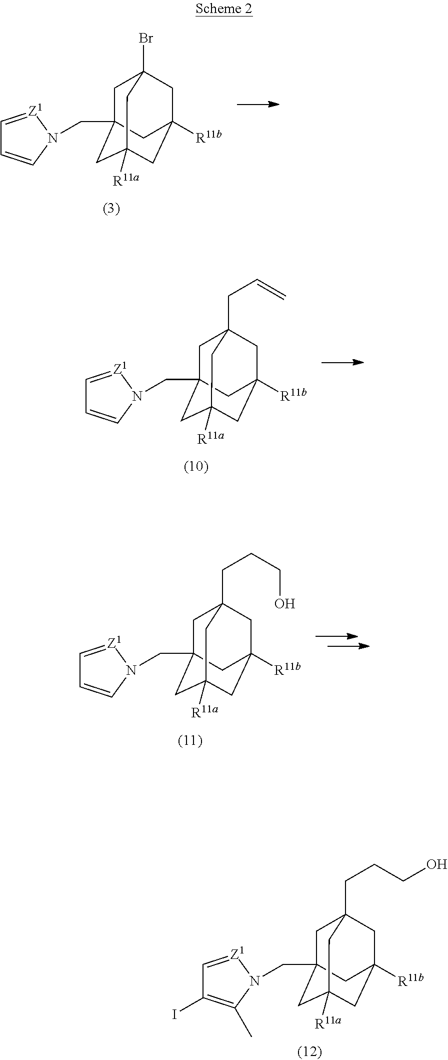

The synthesis of intermediate (12) is described in Scheme 2. Compound (3) can be treated with tri-n-butyl-allylstannane in the presence of ZnCl₂.Et₂O or N, N′-azoisobutyronitrile (AIBN) to provide compound (10) (Yamamoto et al., 1998, Heterocyles 47:765-780). The reaction is typically performed at −78° C. in a solvent, such as, but not limited to dichloromethane. Compound (10) can be treated under standard conditions known in the art for hydroboration/oxidation to provide compound (11). For example, treatment of compound (10) with a reagent such as BH₃.THF in a solvent such as, but not limited to, tetrahydrofuran followed by treatment of the intermediate alkylborane adduct with an oxidant such as, but not limited to, hydrogen peroxide in the presence of a base such as, but not limited to, sodium hydroxide would provide compound (11) (Brown et al., 1968, J. Am. Chem. Soc., 86:397). Typically the addition of BH₃.THF is performed at low temperature before warming to ambient temperature, which is followed by the addition of hydrogen peroxide and sodium hydroxide to generate the alcohol product. Compound (12) can be generated according to Scheme 1, as previously described for compound (9).

5.1.3. Synthesis of Compound (15)

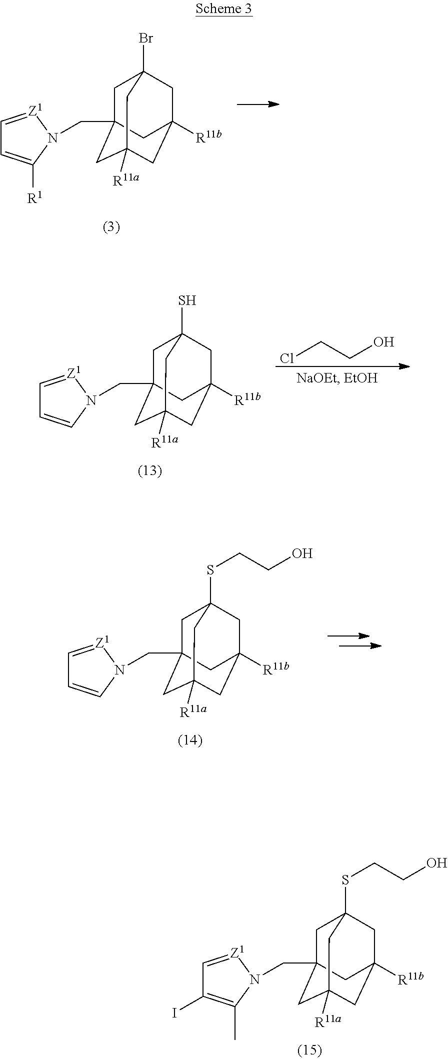

The synthesis of intermediate (15), is described in Scheme 3. Compound (3) can be reacted with thiourea in a solvent mixture of acetic acid and 48% aqueous HBr solution at 100° C. to yield an intermediate that can be subsequently treated with sodium hydroxide in a solvent mixture such as, but not limited to, 20% v/v ethanol in water to provide compound (13). Compound (13) can be reacted with 2-chloroethanol in the presence of a base such as, but not limited to, sodium ethoxide to provide compound (14). The reaction is typically performed at ambient or elevated temperatures in a solvent such as, but not limited to, ethanol. Compound (15) can be generated according to Scheme 1, as previously described for compound (9).

5.1.4. Synthesis of Compound (22)

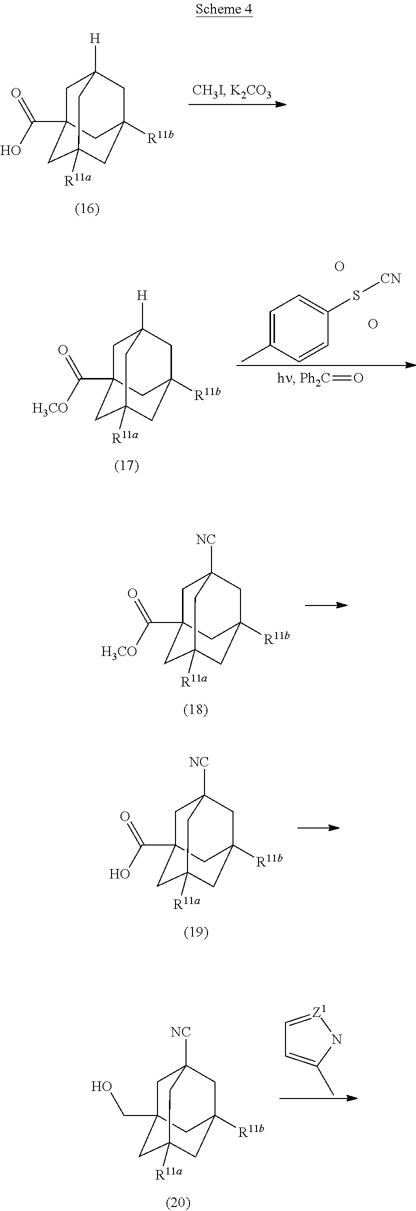

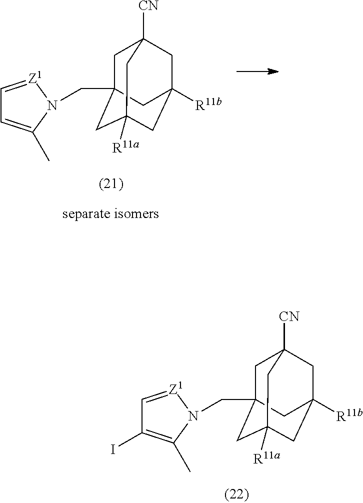

The synthesis of compound (22) is described in Scheme 4. Compound (16) can be reacted with iodomethane in the presence of a base such as, but not limited to, potassium carbonate to provide compound (17). The reaction is typically conducted at ambient or elevated temperature in a solvent such as, but not limited to, acetone or N,N-dimethylformamide. Compound (17) can be reacted under photochemical conditions with tosyl cyanide in the presence of benzophenone to provide compound (18) (see Kamijo et al., Org. Left., 2011, 13:5928-5931). The reaction is typically run at ambient temperature in a solvent such as, but not limited to, acetonitrile or benzene using a Riko 100 W medium pressure mercury lamp as the light source. Compound (18) can be reacted with lithium hydroxide in a solvent system such as, but not limited to, mixtures of water and tetrahydrofuran or water and methanol to provide compound (19). Compound (19) can be treated with BH₃.THF to provide compound (20). The reaction is typically performed at ambient temperature in a solvent, such as, but not limited to, tetrahydrofuran. Compound (21) can be prepared by treating compound (20) with

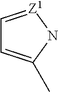

in the presence of cyanomethylenetributylphosphorane. The reaction is typically performed at an elevated temperature in a solvent such as, but not limited to, toluene. Compound (21) can be treated with N-iodosuccinimide to provide compound (22). The reaction is typically performed at ambient temperature is a solvent such as, but not limited to, N,N-dimethylformamide.

5.1.5. Synthesis of Compound (24)

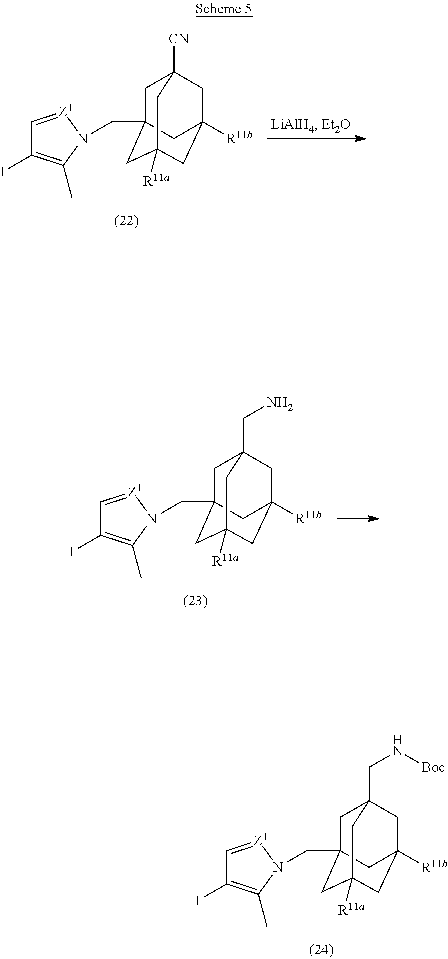

The synthesis of compound (24) is described in Scheme 5. Compound (22) can be treated with a reducing agent such as, but not limited to, lithium aluminum hydride in a solvent such as, but not limited to, diethyl ether or tetrahydrofuran to provide compound (23). Typically the reaction is performed at 0° C. before warming to ambient or elevated temperature. Compound (23) can be reacted with di-tert-butyl dicarbonate under standard conditions described herein or in the literature to provide compound (24).

5.1.6. Synthesis of Compound (24a)

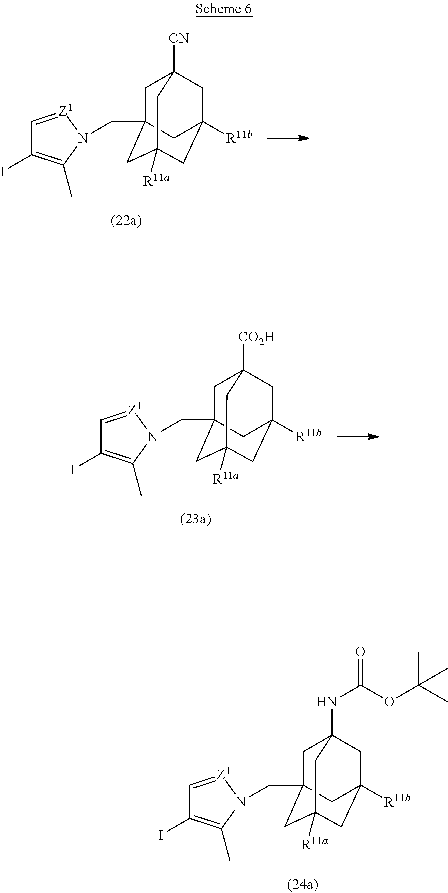

The synthesis of intermediate (24a) is described in Scheme 6. Compound (22a) can be hydrolyzed using conditions described in the literature to provide compound (23a). Typically the reaction is run in the presence of potassium hydroxide in a solvent such as, but not limited to, ethylene glycol at elevated temperatures (see Roberts et al., 1994, J. Org. Chem., 1994, 59:6464-6469; Yang et al, 2013, Org. Lett., 15:690-693). Compound (24a) can be made from compound (23a) by Curtius rearrangement using conditions described in the literature. For example, compound (23a) can be reacted with sodium azide in the presence of tetrabutylammonium bromide, zinc(II) Inflate and di-tert-butyl dicarbonate to provide compound (24a) (see Lebel et al., Org. Lett., 2005, 7:4107-4110). Typically the reaction is run at elevated temperatures, preferably from 40-50° C., in a solvent such as, but not limited to, tetrahydrofuran.

5.1.7. Synthesis of Compound (29)

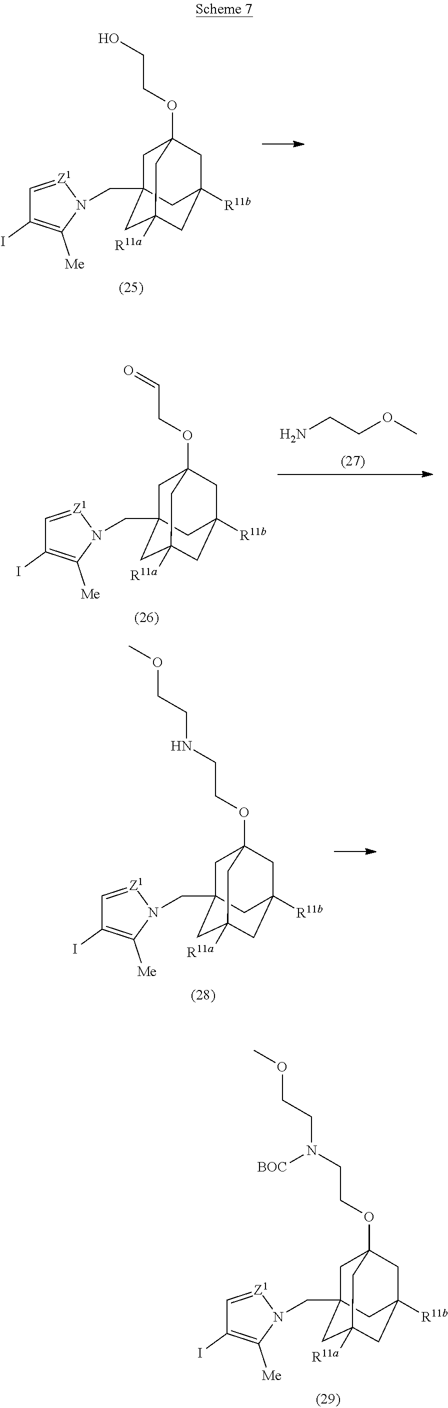

Scheme 7 describes a functionalization of the adamantane ring substituent. Dimethyl sulfoxide can be reacted with oxalyl chloride, followed by the addition of compound (25), in the presence of a base such as, but not limited to triethylamine, to provide compound (26). The reaction is typically performed at low temperature in a solvent such as, but not limited to, dichloromethane. Compound (27) can be reacted with compound (26), followed by treatment with sodium borohydride, to provide compound (28). The reaction is typically performed at ambient temperature in a solvent such as, but not limited to, dichloromethane, methanol, or mixtures thereof. Compound (29) can be prepared by reacting compound (28) with di-tert-butyl dicarbonate, in the presence of N,N-dimethylpyridin-4-amine. The reaction is typically performed at ambient temperature in a solvent such as, but not limited to, tetrahydrofuran.

5.1.8. Synthesis of Compound (35)

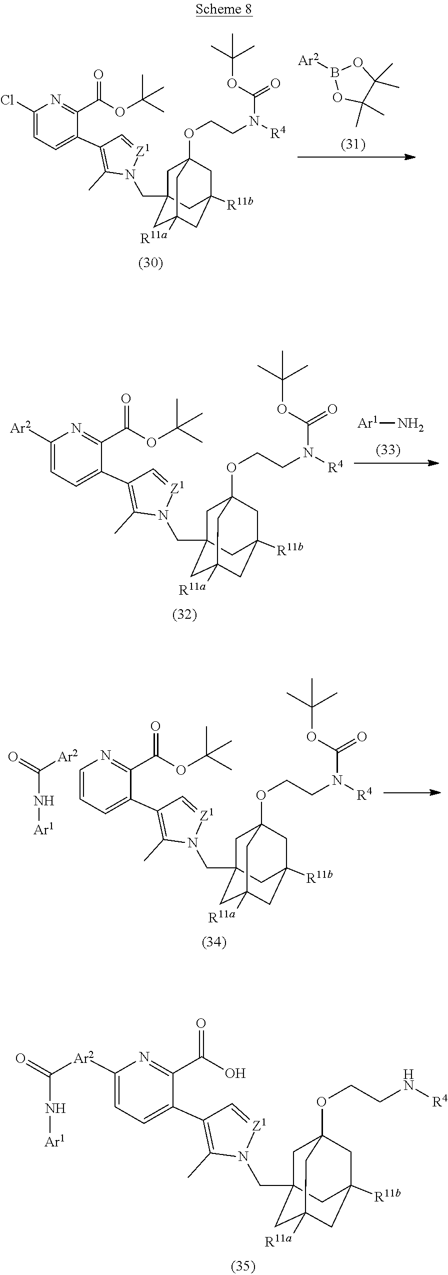

As shown in Scheme 8, compound (30) can be reacted with compound (31) under Suzuki coupling conditions described herein and readily available in the literature, to provide compound (32). Compound (34) can be prepared by reacting compound (32) with compound (33) under conditions described herein, and readily available in the literature. Compound (35) can be prepared by treating compound (34) with an acid such as, but not limited to, trifluoroacetic acid. The reaction is typically performed at ambient temperature in a solvent such as, but not limited to, dichloromethane.

5.1.9. Synthesis of Compound (43)

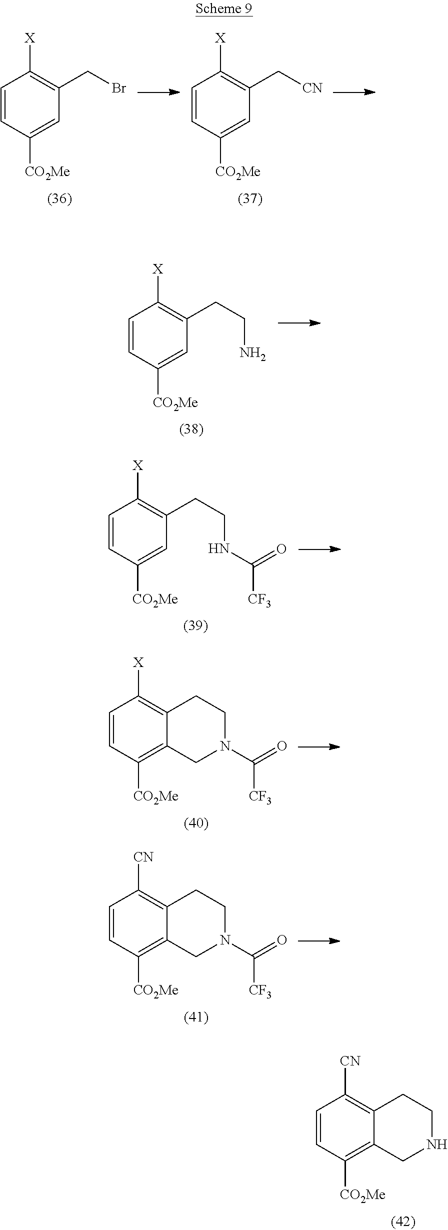

Scheme 9 describes the synthesis of substituted 1,2,3,4-tetrahydroisoquinoline intermediates. Trimethylsilanecarbonitrile can be treated with tetrabutylammonium fluoride and then reacted with compound (36), wherein X is Br or I, to provide compound (37). The additions are typically performed at ambient temperature before heating to an elevated temperature, in a solvent such as, but not limited to, tetrahydrofuran, acetonitrile, or mixtures thereof. Compound (37) can be treated with borane to provide compound (38). The reaction is typically performed at ambient temperature in a solvent such as, but not limited to, tetrahydrofuran. Compound (39) can be prepared by treating compound (38) with trifluoroacetic anhydride, in the presence of a base such as, but no limited to, triethylamine. The reaction is initially performed at low temperature before warming to ambient temperature in a solvent such as, but not limited to, dichloromethane. Compound (39) can be treated with paraformaldehyde in the presence of sulfuric acid to provide compound (40). The reaction is typically performed at ambient temperature. Compound (41) can be prepared by reacting compound (40) with dicyanozinc in the presence of a catalyst such as, but not limited to, tetrakis(triphenylphosphine)palladium(0). The reaction is typically perforated at an elevated temperature under a nitrogen atmosphere in a solvent such as, but not limited to, N,N-dimethylformamide. Compound (41) can be treated with potassium carbonate to provide compound (42). The reaction is typically performed at ambient temperature in a solvent such as, but not limited to, methanol, tetrahydrofuran, water, or mixtures thereof.

5.1.10 Synthesis of Compound (47)

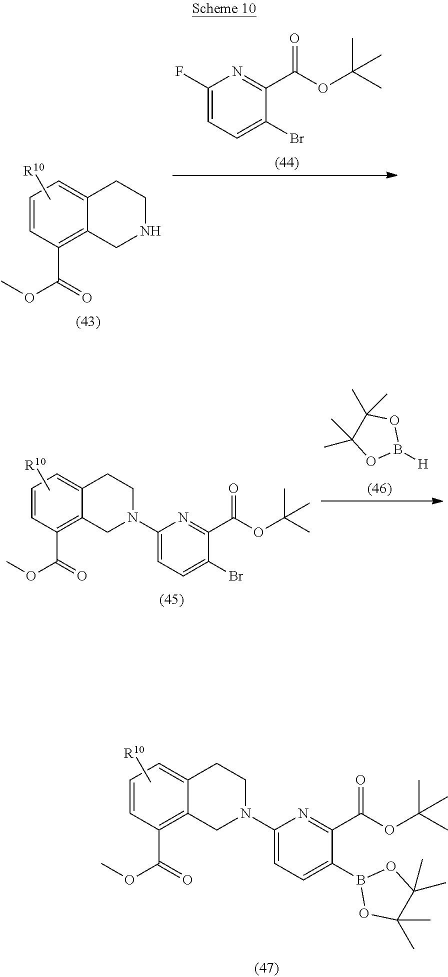

As shown in Scheme 10, compound (45) can be prepared by reacting compound (43), with tert-butyl 3-bromo-6-fluoropicolinate (44) in the presence of a base, such as, but not limited to, N,N-diisopropylethylamine or triethylamine. The reaction is typically performed under an inert atmosphere at an elevated temperature, in a solvent, such as, but not limited to, dimethyl sulfoxide. Compound (45) can be reacted with 4,4,5,5-tetramethyl-1,3,2-dioxaborolane (46), under borylation conditions described herein or in the literature to provide compound (47).

5.1.11. Synthesis of Compound (53)

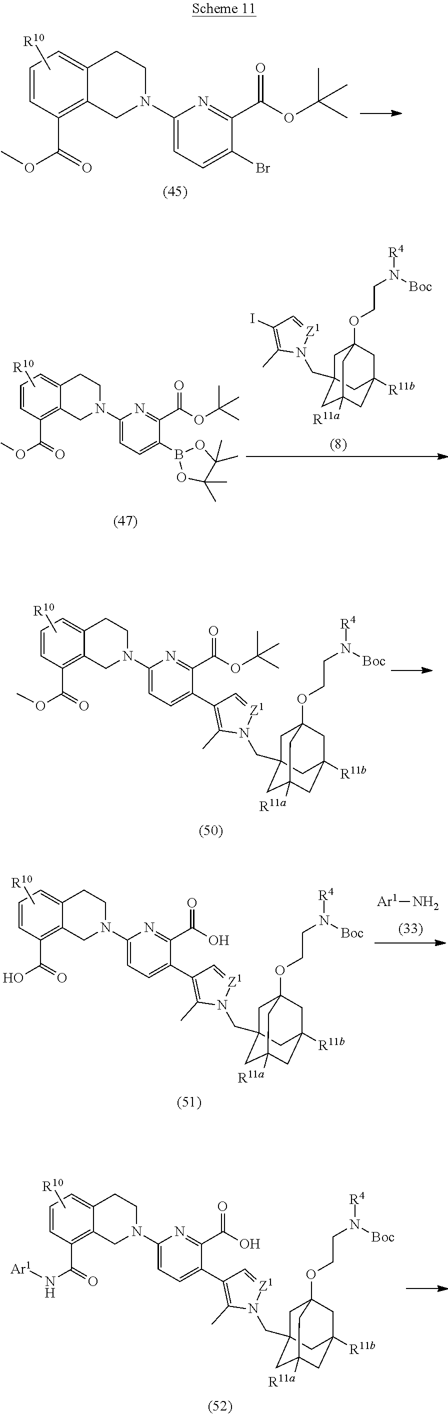

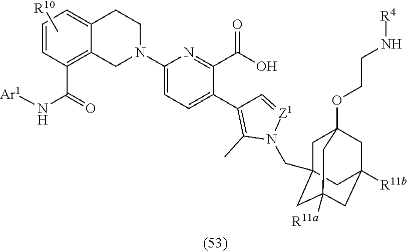

Scheme 11 describes the synthesis of optionally substituted 1,2,3,4-tetrahydroisoquinoline Bcl-xL inhibitors. Compound (47) can be prepared by reacting compound (45) with pinacolborane, in the presence of a base such as but not limited to triethylamine, and a catalyst such as but not limited to [1, 1′-bis(diphenylphosphino)ferrocene]dichloropalladium(II). The reaction is typically performed at an elevated temperature in a solvent such as, but not limited to acetonitrile. Compound (50) can be prepared by reacting compound (47) with compound (8) under Suzuki coupling conditions described herein and readily available in the literature. Compound (50) can be treated with lithium hydroxide to provide compound (51). The reaction is typically performed at ambient temperature in a solvent such as, but not limited to, tetrahydrofuran, methanol, water, or mixtures thereof. Compound (51) can be reacted with compound (33) under amidation conditions described herein and readily available in the literature to provide compound (52). Compound (53) can be prepared by treating compound (52) with an acid such as, but not limited to, trifluoroacetic acid. The reaction is typically performed at ambient temperature in a solvent such as, but not limited to, dichloromethane.

5.1.12. Synthesis of Compound (66)

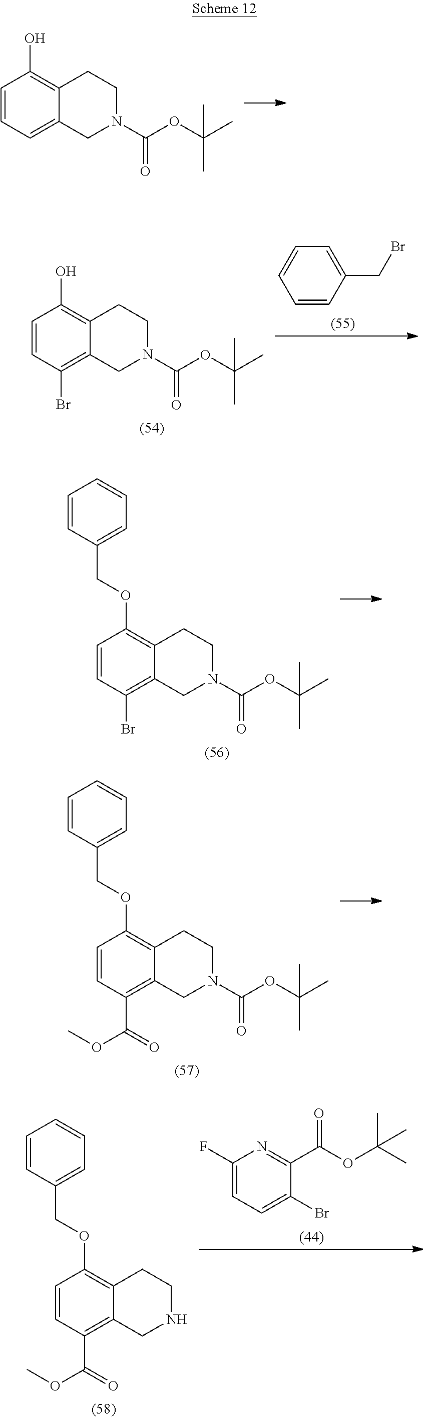

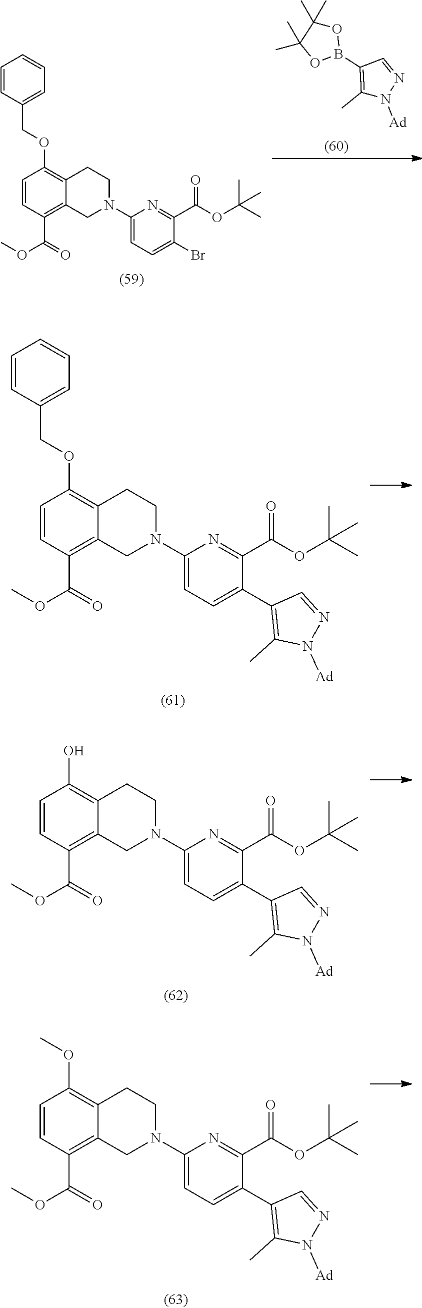

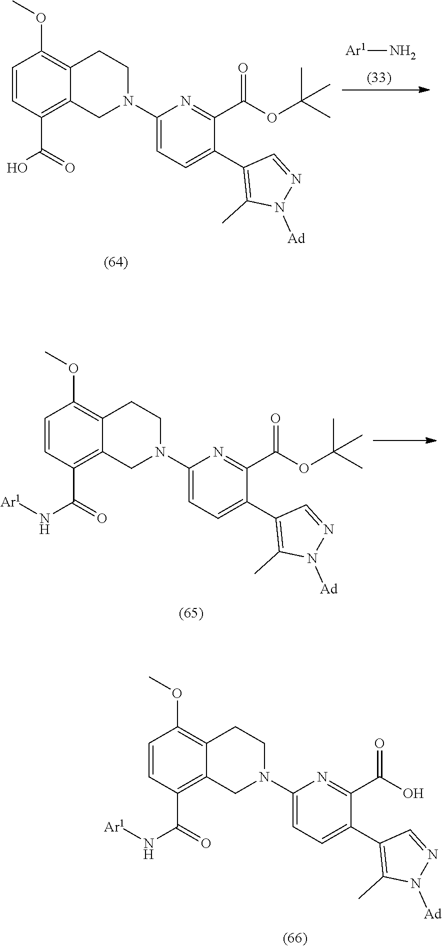

Scheme 12 describes the synthesis of 5-methoxy 1,2,3,4-tetrahydroisoquinoline Bcl-xL inhibitors, tert-Butyl 8-bromo-5-hydroxy-3,4-dihydroisoquinoline-2(1H)-carboxylate (54) can be prepared by treating tert-butyl 5-hydroxy-3,4-dihydroisoquinoline-2(1H)-carboxylate with N-bromosuccinimide. The reaction is typically performed at ambient temperature in a solvent such as, but not limited to N,N-dimethylformamide. Butyl 8-bromo-5-hydroxy-3,4-dihydroisoquinoline-2(1H)-carboxylate (54) can be reacted with benzyl bromide (55) in the presence of a base such as, but not limited to, potassium carbonate to provide tert-butyl 5-(benzyloxy)-8-bromo-3,4-dihydroisoquinoline-2(1H)-carboxylate (56). The reaction is typically performed at an elevated temperature in a solvent such as, but not limited to, acetone, tert-Butyl 5-(benzyloxy)-8-bromo-3,4-dihydroisoquinoline-2(1H)-carboxylate (56) can be treated with carbon monoxide in the presence of methanol and a base such as, but not limited to, triethylamine, and a catalyst such as but not limited to [1,1′-bis(diphenylphosphino)ferrocene]dichloropalladium(II), to provide 2-tert-butyl 8-methyl 5-(benzyloxy)-3,4-dihydroisoquinoline-2.8(1H)-dicarboxylate (57). The reaction is typically performed at an elevated temperature. Methyl 5-(benzyloxy)-1,2,3,4-tetrahydroisoquinoline-8-carboxylate (58) can be prepared by treating 2-tert-butyl 8-methyl 5-(benzyloxy)-3,4-dihydroisoquinoline-2,8(1H)-dicarboxylate (57) with hydrochloric acid. The reaction is typically performed at ambient temperature, in a solvent such as, but not limited to, tetrahydrofuran, dioxane, or mixtures thereof. Methyl 5-(benzyloxy)-1,2,3,4-tetrahydroisoquinoline-8-carboxylate (58) can be reacted with tert-butyl 3-bromo-6-fluoropicolinate (44) in the presence of a base such as, but not limited to, triethylamine, to provide methyl 5-(benzyloxy)-2-(5-bromo-6-(tert-butoxycarbonyl)pyridin-2-yl)-1,2,3,4-tetrahydroisoquinoline-8-carboxylate (59). The reaction is typically performed at elevated temperature in a solvent such as, but not limited to, dimethyl sulfoxide. Methyl 5-(benzyloxy)-2-(5-bromo-6-(tert-butoxycarbonyl)pyridin-2-yl)-1,2,3,4-tetrahydroisoquinoline-8-carboxylate (59) can be reacted with compound (60), wherein Ad is a methyladamantane moiety of the compounds of the disclosure (e.g., the compounds of formula (IIa) and (IIb)) under Suzuki coupling conditions described herein and readily available in the literature, to provide compound (61). Compound (61) can be treated with hydrogen gas in the presence of palladium hydroxide to provide compound (62). The reaction is typically performed at elevated temperature in a solvent such as, but not limited to, tetrahydrofuran. Compound (63) can be prepared by reacting compound (62) with (trimethylsilyl)diazomethane. The reaction is typically performed at ambient temperature, in a solvent such as, but not limited to, dichloromethane, methanol, diethyl ether, or mixtures thereof. Compound (63) can be treated with lithium hydroxide to provide compound (64). The reaction is typically performed at ambient temperature in a solvent such as, but not limited to, tetrahydrofuran, methanol, water, or mixtures thereof. Compound (64) can be reacted with compound (33) under amidation conditions described herein and readily available in the literature to provide compound (65). Compound (66) can be prepared by treating compound (65) with hydrochloric acid. The reaction is typically performed at ambient temperature in a solvent such as, but not limited to, dioxane.

### 5.2 General Methods for Synthesizing Synthons

In the schemes below, the various substituents Ar¹, Ar², Z¹, R⁴, R^{11a} and R^{11b} are as defined in the Detailed Description section.

5.2.1. Synthesis of Compound (89)

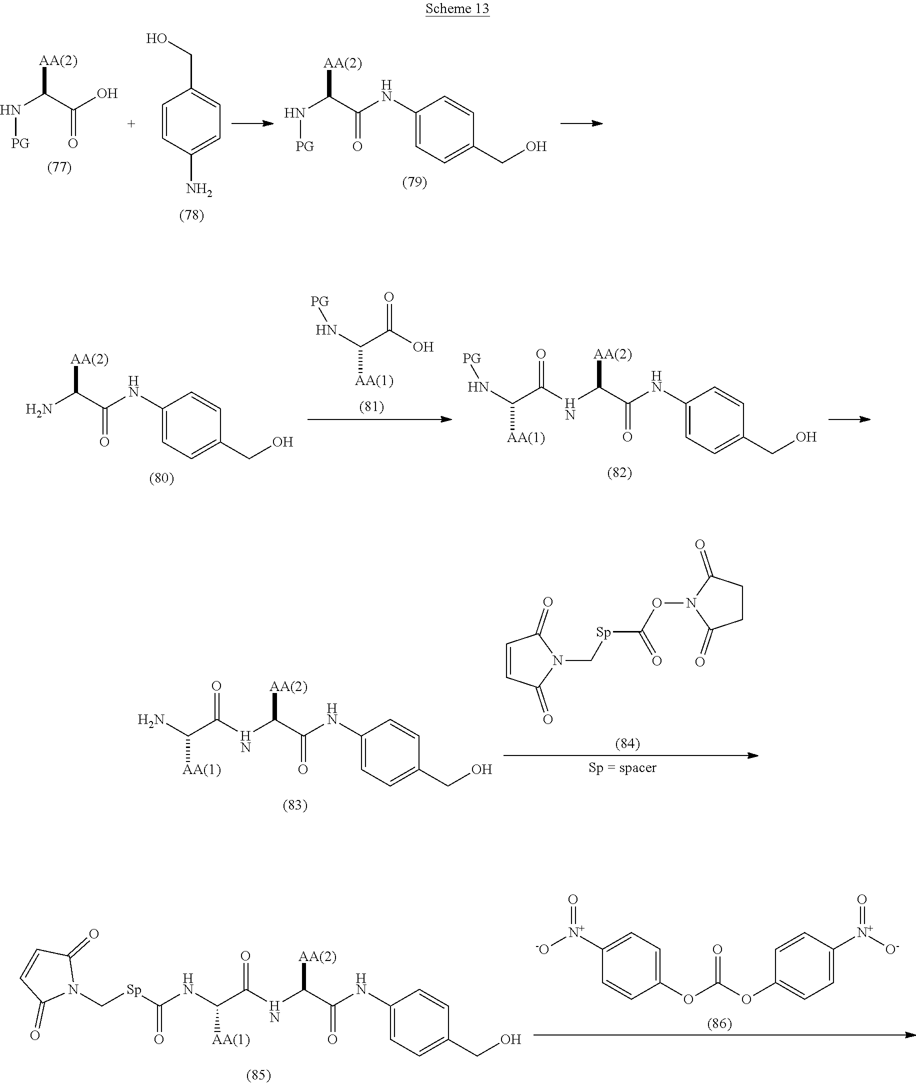

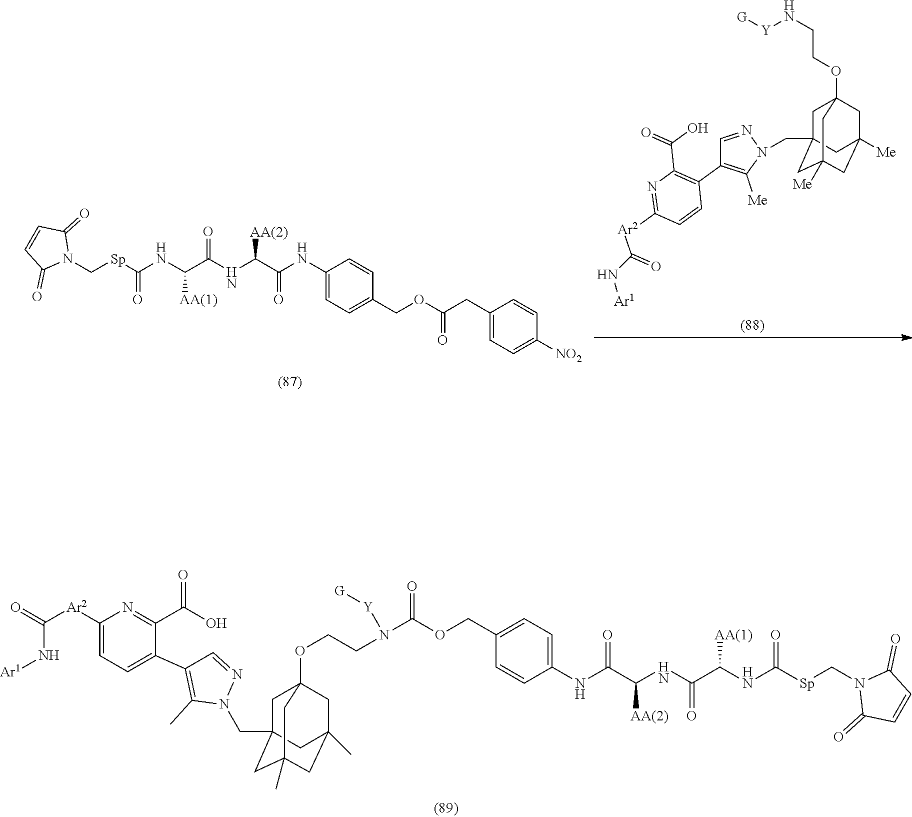

As shown in scheme 13, compounds of formula (77), wherein PC is an appropriate base labile protecting group and AA(2) is Cit, Ala, or Lys, can be reacted with 4-(aminophenyl)methanol (78), under amidation conditions described herein or readily available in the literature to provide compound (79). Compound (80) can be prepared by reacting compound (79) with a base such as, but not limited to, diethylamine. The reaction is typically performed at ambient temperature in a solvent such as but not limited to N,N-dimethylformamide. Compound (81), wherein PC is an appropriate base or acid labile protecting group and AA(1) is Val or Phe, can be reacted with compound (80), under amidation conditions described herein or readily available in the literature to provide compound (82). Compound (83) can be prepared by treating compound (82) with diethylamine or trifluoroacetic acid, as appropriate. The reaction is typically performed at ambient temperature in a solvent such as but not limited to dichloromethane. Compound (84), wherein Sp is a spacer, can be reacted with compound (83) to provide compound (85). The reaction is typically performed at ambient temperature in a solvent such as but not limited to N,N-dimethylformamide. Compound (85) can be reacted with bis(4-nitrophenyl) carbonate (86) in the presence of a base such as, but not limited to N,N-diisopropylethylamine, to provide compounds (87). The reaction is typically performed at ambient temperature in a solvent such as but not limited to N,N-dimethylformamide. Compounds (87) can be reacted with compounds of formula (88) in the presence of a base such as, but not limited to, N,N-diisopropylethylamine, to provide compound (89). The reaction is typically performed at ambient temperature in a solvent such as, but not limited to, N,N-dimethylformamide.

5.2.2. Synthesis of Compounds (94) and (96)

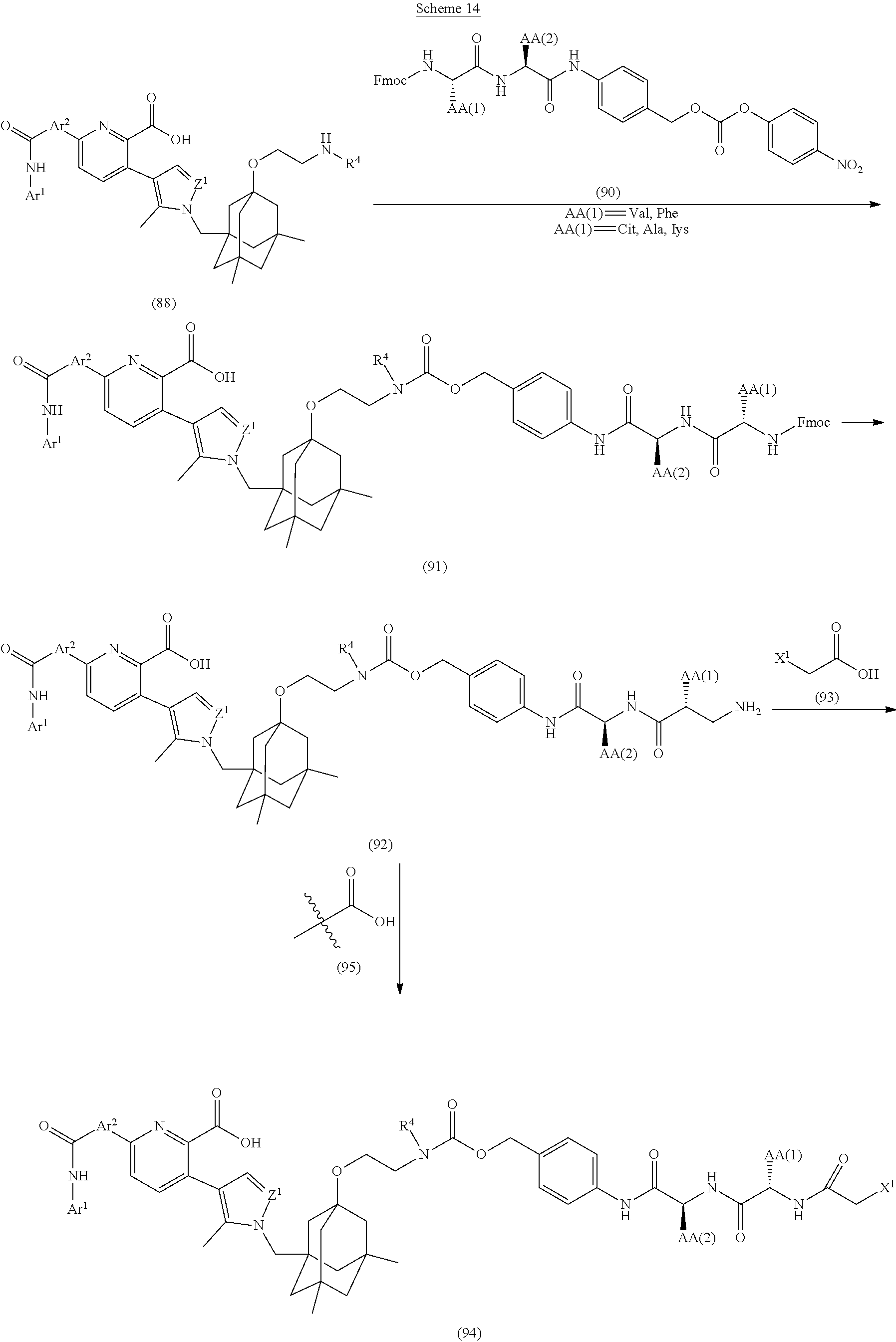

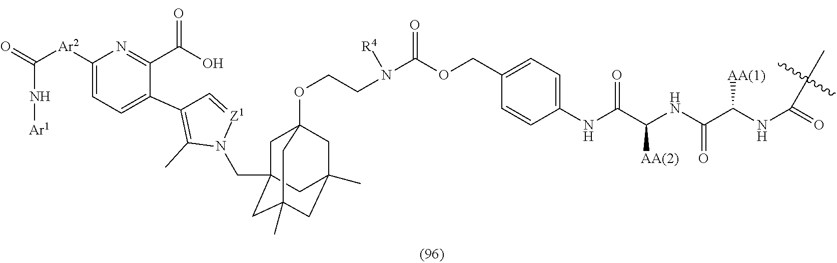

Scheme 14 describes the installment of alternative mAb-linker attachments to dipeptide synthons. Compound (88), wherein can be reacted with compound (90) in the presence of a base such as, but not limited to, N-ethyl-N-isopropylpropan-2-amine, to provide compound (91). The reaction is typically performed at ambient temperature in a solvent such as but not limited to N,N-dimethylformamide. Compound (92) can be prepared by reacting compound (91) with diethylamine. The reaction is typically performed at ambient temperature in a solvent such as but not limited to N,N-dimethylformamide. Compound (93), wherein X¹ is Cl, Br, or I, can be reacted with compound (92), under amidation conditions described herein or readily available in the literature to provide compound (94). Compound (92) can be reacted with compounds of formula (95) under amidation conditions described herein or readily available in the literature to provide compound (96).

5.2.3. Synthesis of Compound (1061

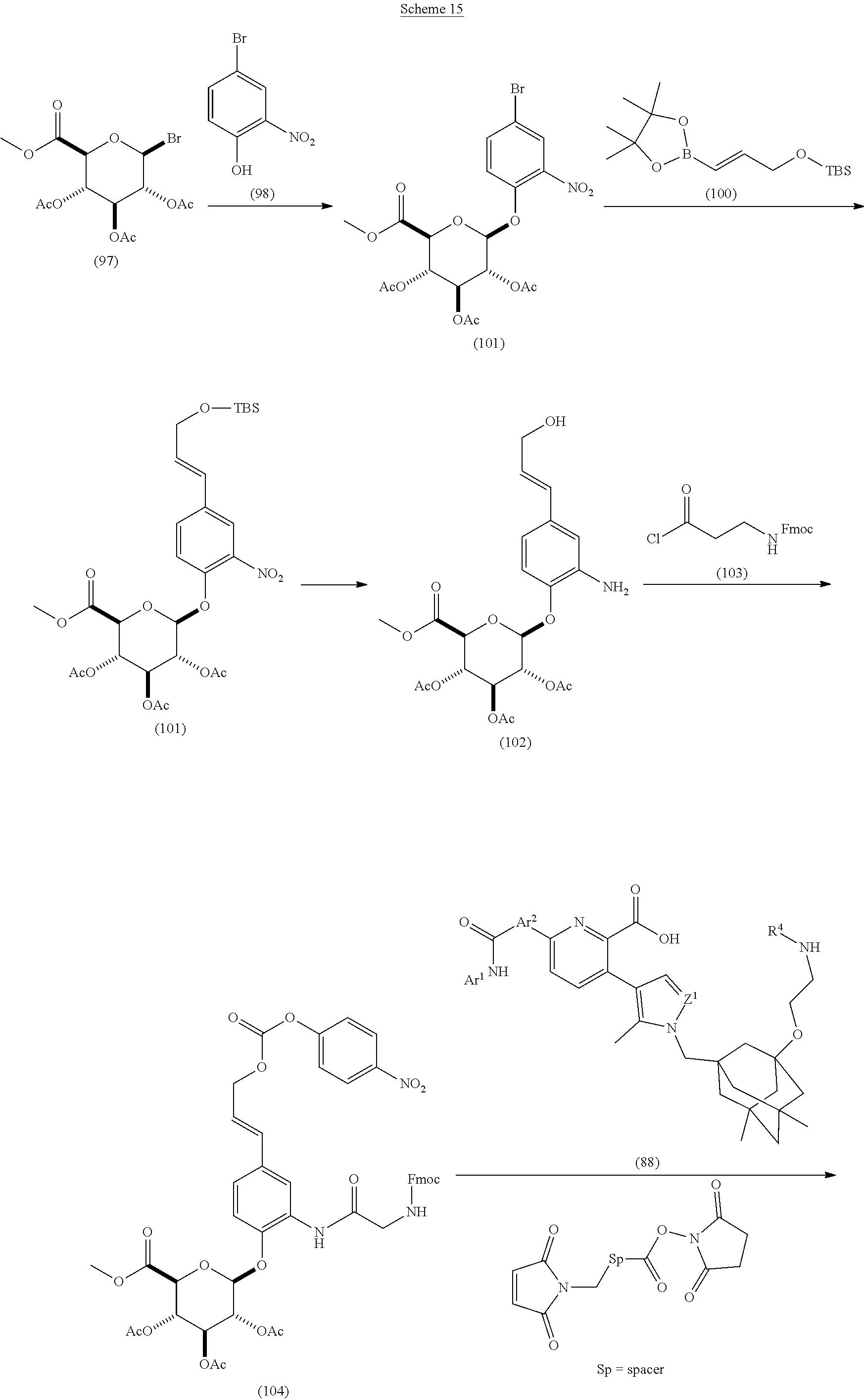

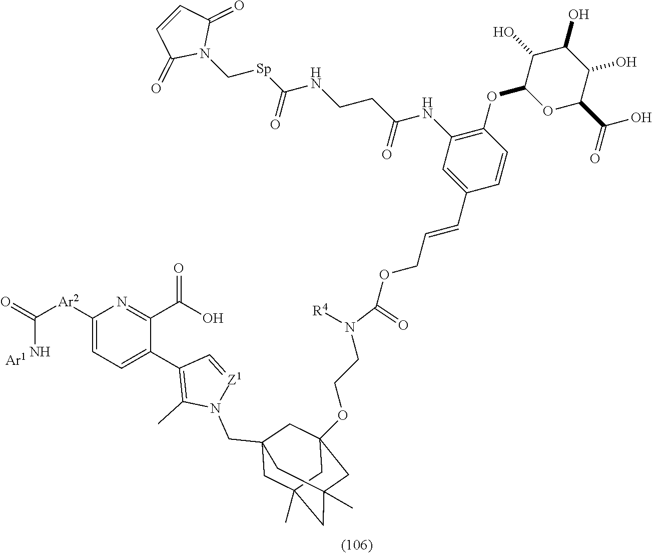

Scheme 15 describes the synthesis of vinyl glucuronide linker intermediates and synthons. (2R,3R,4S,5S,6S)-2-Bromo-6-(methoxycarbonyl)tetrahydro-2H-pyran-3,4,5-triyl triacetate (97) can be treated with silver oxide, followed by 4-bromo-2-nitrophenol (98) to provide (2S,3R,4S,5S,6S)-2-(4-bromo-2-nitrophenoxy)-6-(methoxycarbonyl)tetrahydro-2H-pyran-3,4,5-triyl triacetate (99). The reaction is typically performed at ambient temperature in a solvent, such as, but not limited to, acetonitrile. (2S,3R,4S,5S,6S)-2-(4-Bromo-2-nitrophenoxy)-6-(methoxycarbonyl)tetrahydro-2H-pyran-3,4,5-triyl triacetate (99) can be reacted with (E)-tert-butyldimethyl((3-(4,4,5,5-tetramethyl-1,3,2-dioxaborolan-2-yl)alkyl)oxy)silane (100) in the presence of a base such as, but not limited to, sodium carbonate, and a catalyst such as but not limited to tris(dibenzylideneacetone)dipalladium (Pd2(dba)3), to provide (2S,3R,4S,5S,6S)-2-(4-((E)-3-((tert-butyldimethylsilyl)oxy)prop-1-en-1-yl)-2-nitrophenoxy)-6-(methoxycarbonyl)tetrahydro-2H-pyran-3,4,5-triyl triacetate (101). The reaction is typically performed at an elevated temperature in a solvent, such as, but not limited to, tetrahydrofuran. (2S,3R,4S,5S,6S)-2-(2-amino-4-((E)-3-hydroxyprop-1-en-1-yl)phenoxy)-6-(methoxycarbonyl)tetrahydro-2H-pyran-3,4,5-triyl triacetate (102) can be prepared by reacting (2S,3R,4S,5S,6S)-2-(4-((E)-3-((tert-butyldimethylsilyl)oxy)prop-1-en-1-yl)-2-nitrophenoxy)-6-(methoxycarbonyl)tetrahydro-2H-pyran-3,4,5-triyl triacetate (101) with zinc in the presence of an acid such as, but not limited to, hydrochloric acid. The addition is typically performed at low temperature before warming to ambient temperature in a solvent such as, but not limited to, tetrahydrofuran, water, or mixtures thereof. (2S,3R,4S,5S,6S)-2-(2-amino-4-((E)-3-hydroxyprop-1-en-1-yl)phenoxy)-6-(methoxycarbonyl)tetrahydro-2H-pyran-3,4,5-triyl triacetate (102) can be reacted with (9H-fluoren-9-yl)methyl (3-chloro-3-oxopropyl)carbamate (103), in the presence of a base such as, but not limited to, N,N-diisopropylethylamine, to provide (2S,3R,4S,5S,6S)-2-(2-(3-((((9H-fluoren-9-yl)methoxy)carbonyl)amino)propanamido)-4-((E)-3-hydroxyprop-1-en-1-yl)phenoxy)-6-(methoxycarbonyl)tetrahydro-2H-pyran-3,4,5-triyl triacetate (104). The addition is typically performed at low temperature before warming to ambient temperature in a solvent such as, but not limited to, dichloromethane. Compound (88) can be reacted with (2S,3R,4S,5S,6S)-2-(2-(3-((((9H-fluoren-9-yl)methoxy)carbonyl)amino)propanamido)-4-((E)-3-hydroxyprop-1-en-1-yl)phenoxy)-6-(methoxycarbonyl)tetrahydro-2H-pyran-3,4,5-triyl triacetate (104) in the presence of a base such as, but not limited to, N-ethyl-N-isopropylpropan-2-amine, followed by work up and reaction with compound (105) in the presence of a base such as, but not limited to, N,N-diisopropylethylamine to provide compound (106). The reactions are typically performed at ambient temperature in a solvent such as, but not limited to N,N-dimethylformamide.

5.2.4. Synthesis of Compound (115)

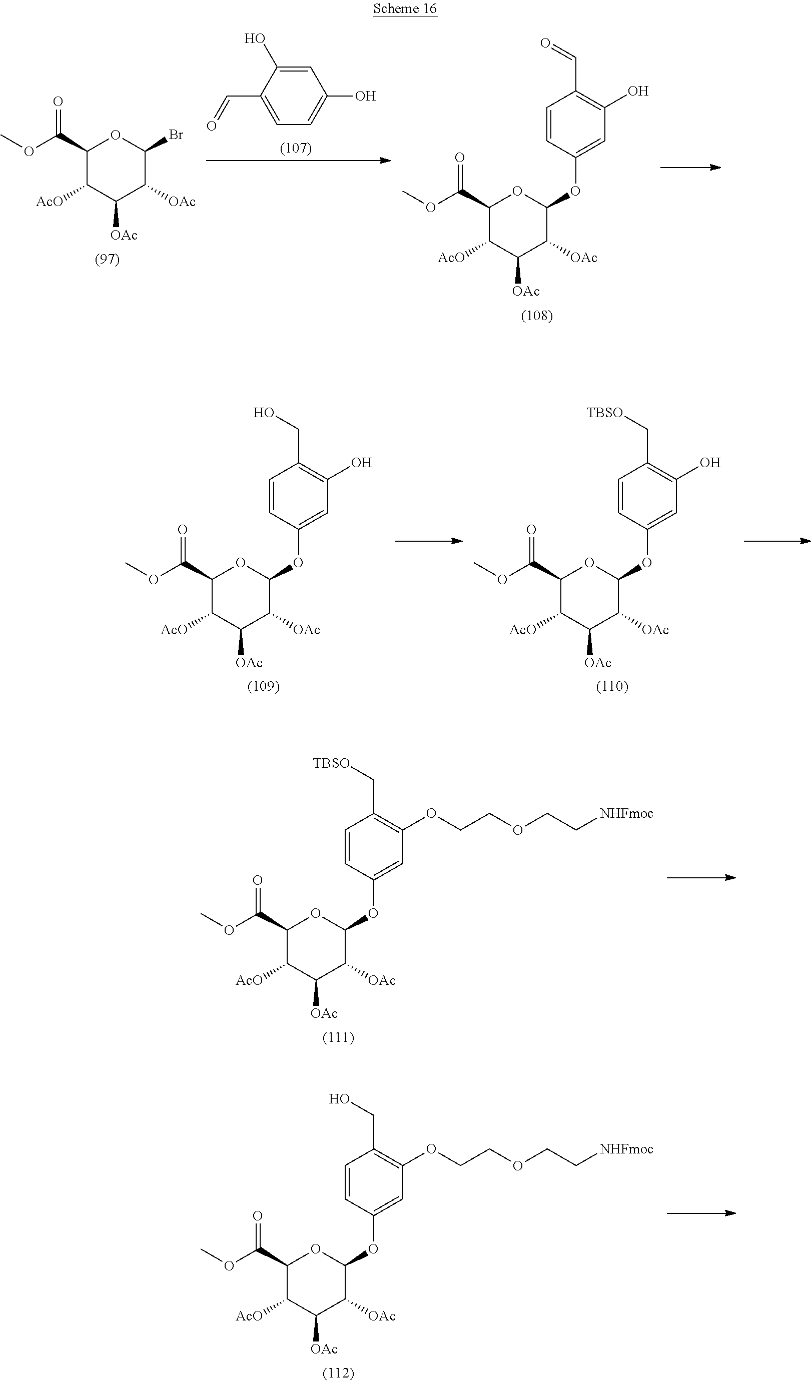

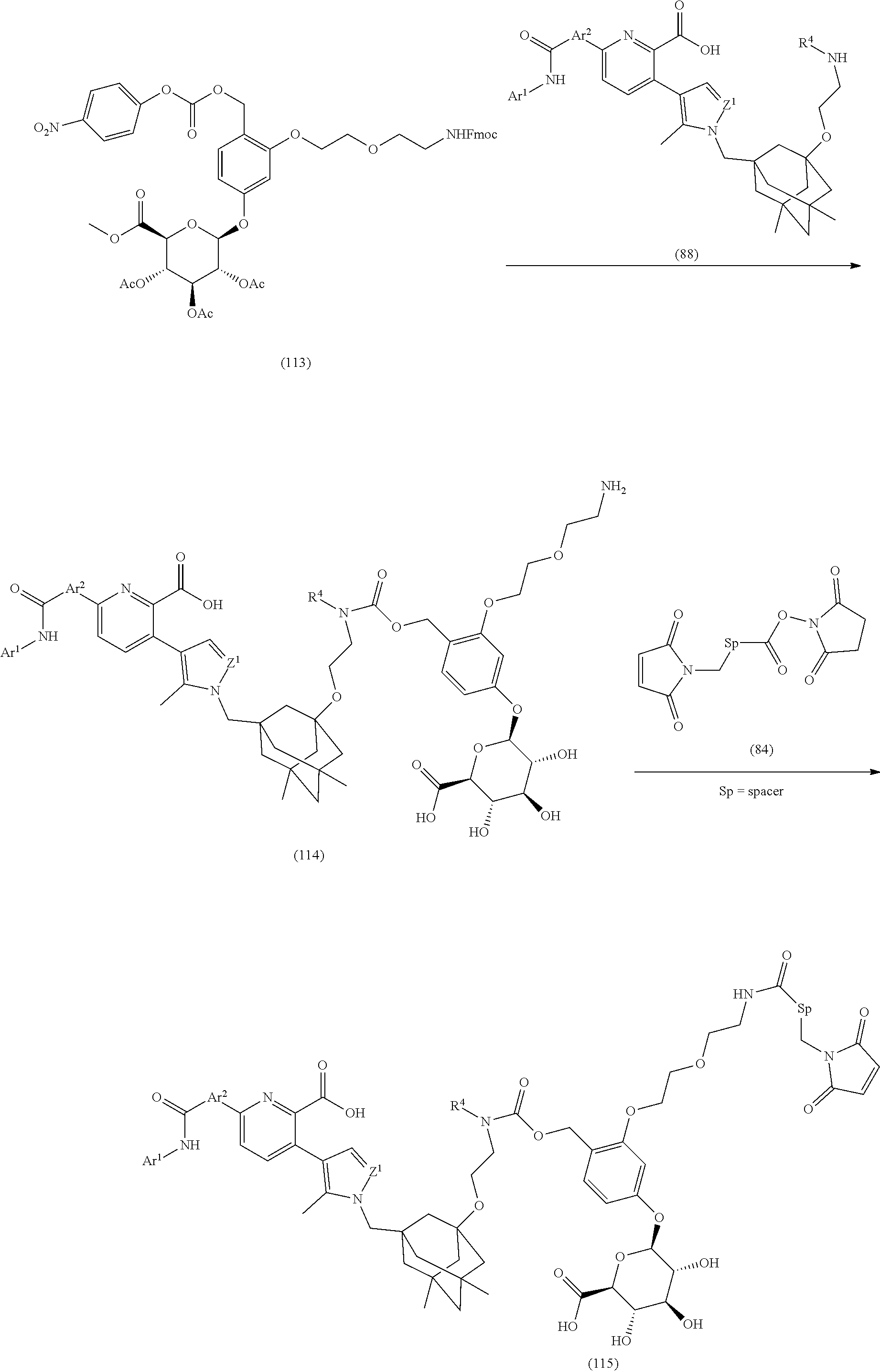

Scheme 16 describes the synthesis of a representative 2-ether glucuronide linker intermediate and synthon. (2S,3R,4S,5S,6S)-2-Bromo-6-(methoxycarbonyl)tetrahydro-2H-pyran-3,4,5-triyl triacetate (97) can be reacted with 2,4-dihydroxybenzaldehyde (107) in the presence of silver carbonate to provide (2S,3R,4S,5S,6S)-2-(4-formyl-3-hydroxyphenoxy)-6-(methoxycarbonyl)tetrahydro-2H-pyran-3,4,5-triyl triacetate (108). The reaction is typically performed at an elevated temperature in a solvent, such as, but not limited to, acetonitrile. (2S,3R,4S,5S,6S)-2-(4-Formyl-3-hydroxyphenoxy)-6-(methoxycarbonyl)tetrahydro-2H-pyran-3,4,5-triyl triacetate (108) can be treated with sodium borohydride to provide (2S,3R,4S,5S,6S)-2-(3-hydroxy-4-(hydroxymethyl)phenoxy)-6-(methoxycarbonyl)tetrahydro-2H-pyran-3,4,5-triyl triacetate (109). The addition is typically performed at low temperature before warming to ambient temperature in a solvent such as but not limited to tetrahydrofuran, methanol, or mixtures thereof. (2S,3R,4S,5S,6S)-2-(4-(((tert-butyldimethylsilyl)oxy)methyl)-3-hydroxyphenoxy)-6-(methoxycarbonyl)tetrahydro-2H-pyran-3,4,5-triyl triacetate (110) can be prepared by reacting (2S,3R,4S,5S,6S)-2-(3-hydroxy-4-(hydroxymethyl)phenoxy)-6-(methoxycarbonyl)tetrahydro-2H-pyran-3,4,5-triyl triacetate (109) with tert-butyldimethylsilyl chloride in the presence of imidazole. The reaction is typically performed at low temperature in a solvent, such as, but not limited to, dichloromethane. (2S,3R,4S,5S,6S)-2-(3-(2-(2-((((9H-Fluoren-9-yl)methoxy)carbonyl)amino)ethoxy)ethoxy)-4-(((tert-butyldimethylsilyl)oxy)methyl)phenoxy)-6-(methoxycarbonyl)tetrahydro-2H-pyran-3,4,5-triyl triacetate (111) can be prepared by reacting (2S,3R,4S,5S,6S)-2-(4-(((tert-butyldimethylsilyl)oxy)methyl)-3-hydroxyphenoxy)-6-(methoxycarbonyl)tetrahydro-2H-pyran-3,4,5-triyl triacetate (110) with (9H-fluoren-9-yl)methyl (2-(2-hydroxyethoxy)ethyl)carbamate in the presence of triphenylphosphine and a azodicarboxylate such as, but not limited to, di-tert-butyl diazene-1,2-dicarboxylate. The reaction is typically performed at ambient temperature in a solvent such as but not limited to toluene. (2S,3R,4S,5S,6S)-2-(3-(2-(2-((((9H-Fluoren-9-yl)methoxy)carbonyl)amino)ethoxy)ethoxy)-4-(((tert-butyldimethylsilyl)oxy)methyl)phenoxy)-6-(methoxycarbonyl)tetrahydro-2H-pyran-3,4,5-triyl triacetate (111) can be treated with acetic acid to provide (2S,3R,4S,5S,6S)-2-(3-(2-(2-((((9H-fluoren-9-yl)methoxy)carbonyl)amino)ethoxy)ethoxy)-4-(hydroxymethyl)phenoxy)-6-(methoxycarbonyl)tetrahydro-2H-pyran-3,4,5-triyl triacetate (112). The reaction is typically performed at ambient temperature in a solvent such as but not limited to water, tetrahydrofuran, or mixtures thereof. (2S,3R,4S,5S,6S)-2-(3-(2-(2-((((9H-Fluoren-9-yl)methoxy)carbonyl)amino)ethoxy)ethoxy)-4-((((4-nitrophenoxy)carbonyl)oxy)methyl)phenoxy)-6-(methoxycarbonyl)tetrahydro-2H-pyran-3,4,5-triyl triacetate (113) can be prepared by reacting (2S,3R,4S,5S,6S)-2-(3-(2-(2-((((9H-fluoren-9-yl)methoxy)carbonyl)amino)ethoxy)ethoxy)-4-(hydroxymethyl)phenoxy)-6-(methoxycarbonyl)tetrahydro-2H-pyran-3,4,5-triyl triacetate (91) with bis(4-nitrophenyl) carbonate in the presence of a base such as but not limited to N-ethyl-N-isopropylpropan-2-amine. The reaction is typically performed at ambient temperature in a solvent such as but not limited to N,N-dimethylformamide. (2S,3R,4S,5S,6S)-2-(3-(2-(2-((((9H-Fluoren-9-yl)methoxy)carbonyl)amino)ethoxy)ethoxy)-4-((((4-nitrophenoxy)carbonyl)oxy)methyl)phenoxy)-6-(methoxycarbonyl)tetrahydro-2H-pyran-3,4,5-triyl triacetate (113) can be treated with compound (88) in the presence of a base such as but not limited to N-ethyl-N-isopropylpropan-2-amine, followed by treatment with lithium hydroxide to provide a compound (114). The reaction is typically performed at ambient temperature in a solvent such as but not limited to N,N-dimethylformamide, tetrahydrofuran, methanol, or mixtures thereof. Compound (115) can be prepared by reacting compound (114) with compound (84) in the presence of a base such as but not limited to N-ethyl-N-isopropylpropan-2-amine. The reaction is typically performed at ambient temperature in a solvent such as but not limited to N,N-dimethylformamide.

5.2.5. Synthesis of Compound (119)

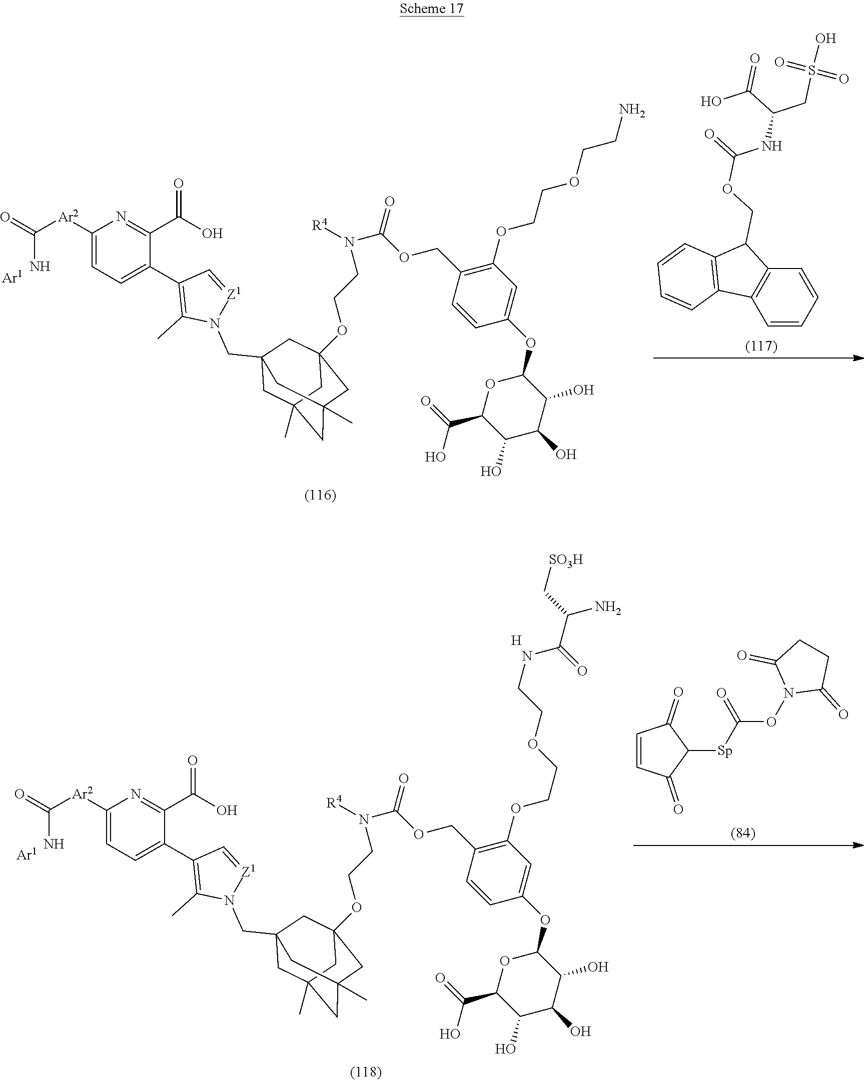

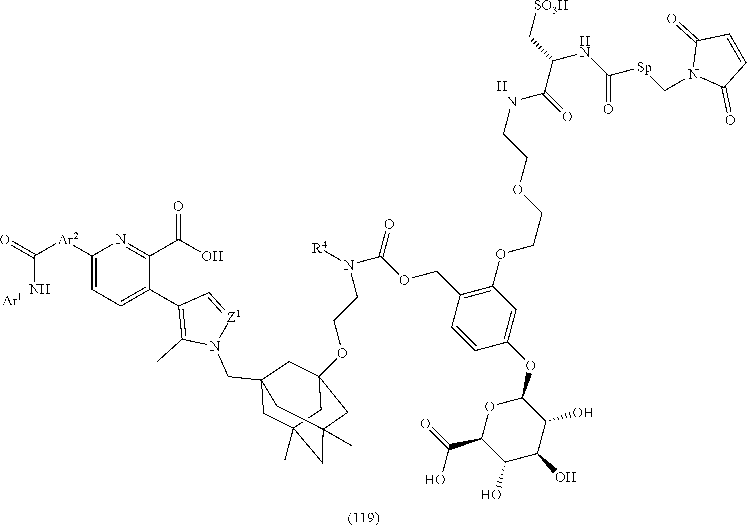

Scheme 17 describes the introduction of a second solubilizing group to a sugar linker. Compound (116) can be reacted with (R)-2-((((9H-fluoren-9-yl)methoxy)carbonyl)amino)-3-sulfopropanoic acid (117), under amidation conditions described herein or readily available in the literature, followed by treatment with a base such as but not limited to diethylamine, to provide compound (118). Compound (118) can be reacted with compound (84), wherein Sp is a spacer, under amidation conditions described herein or readily available in the literature, to provide compound (119).

5.2.6. Synthesis of Compound (129)

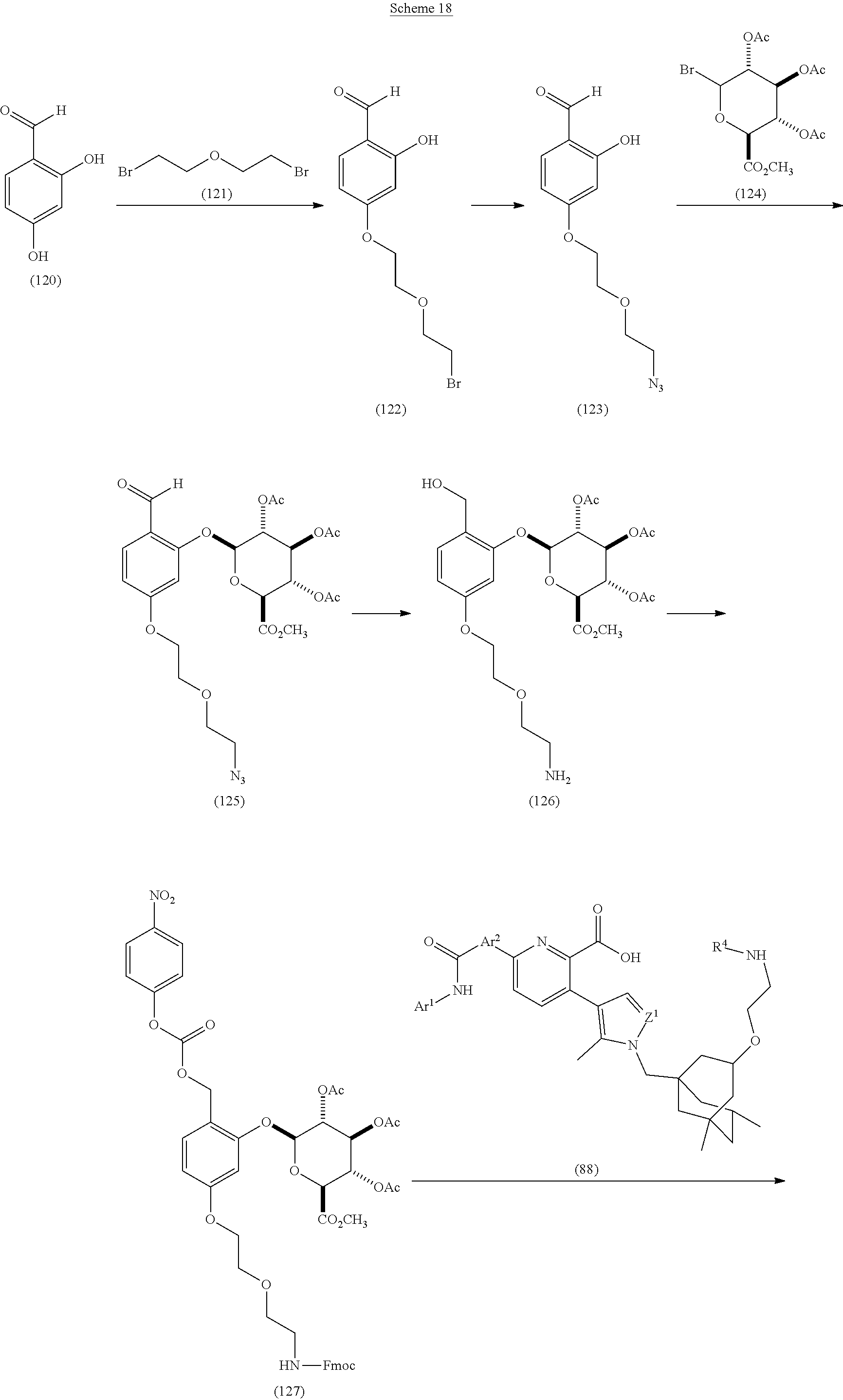

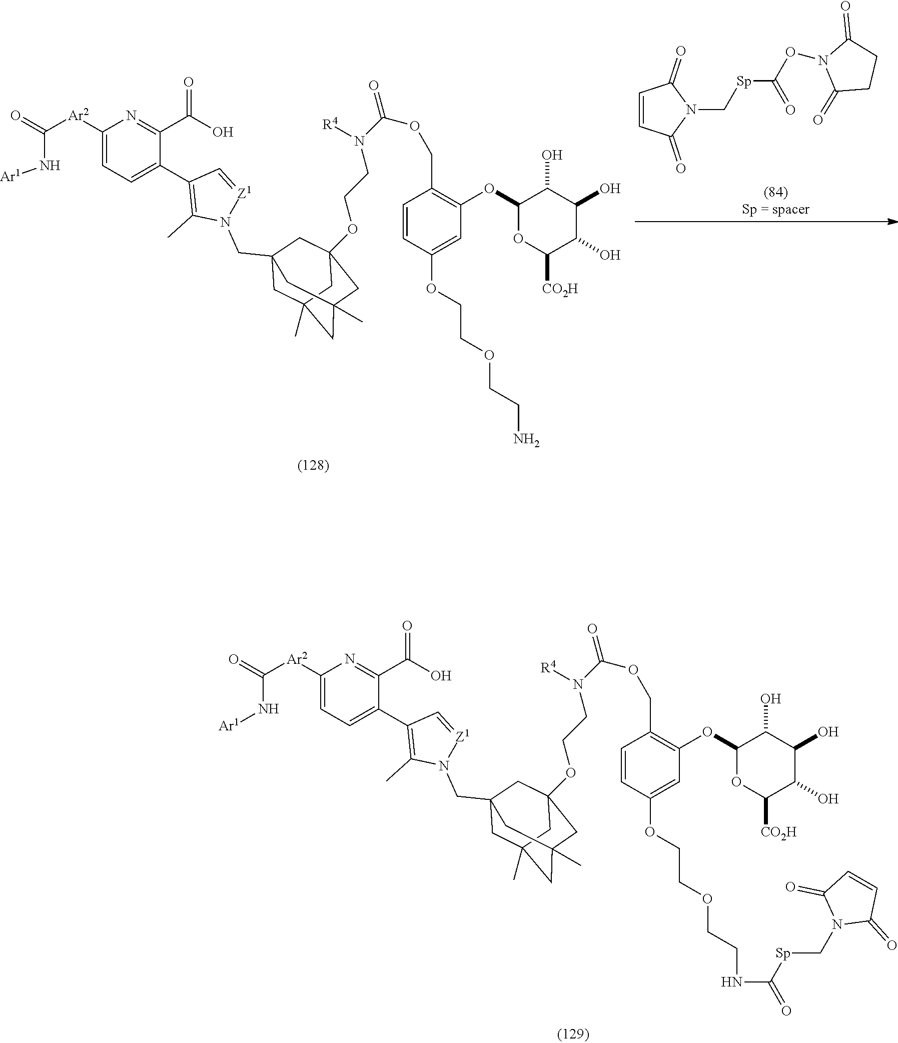

Scheme 18 describes the synthesis of 4-ether glucuronide linker intermediates and synthons, 4-(2-(2-Bromoethoxy)ethoxy)-2-hydroxybenzaldehyde (122) can be prepared by reacting 2,4-dihydroxybenzaldehyde (120) with 1-bromo-2-(2-bromoethoxy)ethane (121) in the presence of a base such as, but not limited to, potassium carbonate. The reaction is typically performed at an elevated temperature in a solvent such as but not limited to acetonitrile. 4-(2-(2-Bromoethoxy)ethoxy)-2-hydroxybenzaldehyde (122) can be treated with sodium azide to provide 4-(2-(2-azidoethoxy)ethoxy)-2-hydroxybenzaldehyde (123). The reaction is typically performed at ambient temperature in a solvent such as but not limited to N,N-dimethylformamide. (2S,3R,4S,5S,6S)-2-(5-(2-(2-Azidoethoxy)ethoxy)-2-formylphenoxy)-6-(methoxycarbonyl)tetrahydro-2H-pyran-3,4,5-triyl triacetate (125) can be prepared by reacting 4-(2-(2-azidoethoxy)ethoxy)-2-hydroxybenzaldehyde (123) with (3R,4S,5S,6S)-2-bromo-6-(methoxycarbonyl)tetrahydro-2H-pyran-3,4,5-triyl triacetate (124) in the presence of silver oxide. The reaction is typically performed at ambient temperature in a solvent such as, but not limited to, acetonitrile. Hydrogenation of (2S,3R,4S,5S,6S)-2-(5-(2-(2-azidoethoxy)ethoxy)-2-formylphenoxy)-6-(methoxycarbonyl)tetrahydro-2H-pyran-3,4,5-triyl triacetate (125) in the presence of Pd/C will provide (2S,3R,4S,5S,6S)-2-(5-(2-(2-aminoethoxy)ethoxy)-2-(hydroxymethyl)phenoxy)-6-(methoxycarbonyl)tetrahydro-2H-pyran-3,4,5-triyl triacetate (126). The reaction is typically performed at ambient temperature in a solvent such as, but not limited to, tetrahydrofuran. (2S,3R,4S,5S,6S)-2-(5-(2-(2-((((9H-Fluoren-9-yl)methoxy)carbonyl)amino)ethoxy)ethoxy)-2-(hydroxymethyl)phenoxy)-6-(methoxycarbonyl)tetrahydro-2H-pyran-3,4,5-triyl triacetate (127) can be prepared by treating (2S,3R,4S,5S,6S)-2-(5-(2-(2-aminoethoxy)ethoxy)-2-(hydroxymethyl)phenoxy)-6-(methoxycarbonyl)tetrahydro-2H-pyran-3,4,5-triyl triacetate (126) with (9H-fluoren-9-yl)methyl carbonochloridate in the presence of a base, such as, but not limited to, N-ethyl-N-isopropylpropan-2-amine. The reaction is typically performed at low temperature in a solvent such as, but not limited to, dichloromethane. Compound (88) can be reacted with (2S,3R,4S,5S,6S)-2-(5-(2-(2-((((9H-Fluoren-9-yl)methoxy)carbonyl)amino)ethoxy)ethoxy)-2-(hydroxymethyl)phenoxy)-6-(methoxycarbonyl)tetrahydro-2H-pyran-3,4,5-triyl triacetate (127) in the presence of a base, such as, but not limited to, N-ethyl-N-isopropylpropan-2-amine, followed by treatment with lithium hydroxide to provide compound (128). The reaction is typically performed at low temperature in a solvent such as, but not limited to, N,N-dimethylformamide. Compound (129) can be prepared by reacting compound (128) with compound (84) in the presence of a base such as, but not limited to, N-ethyl-N-isopropylpropan-2-amine. The reaction is typically performed at ambient temperature in a solvent such as but not limited to N,N-dimethylformamide.

5.2.7. Synthesis of Compound (139)

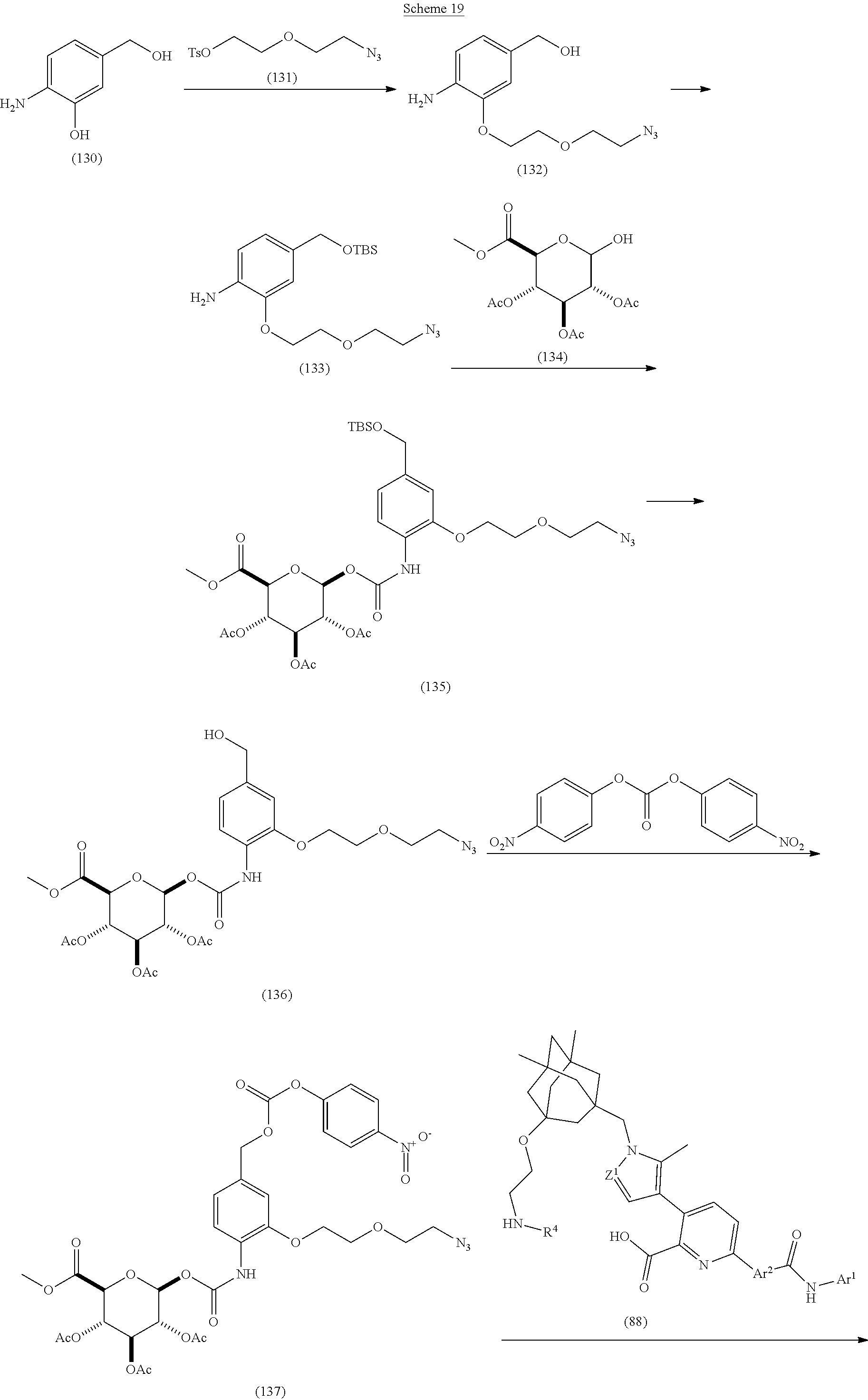

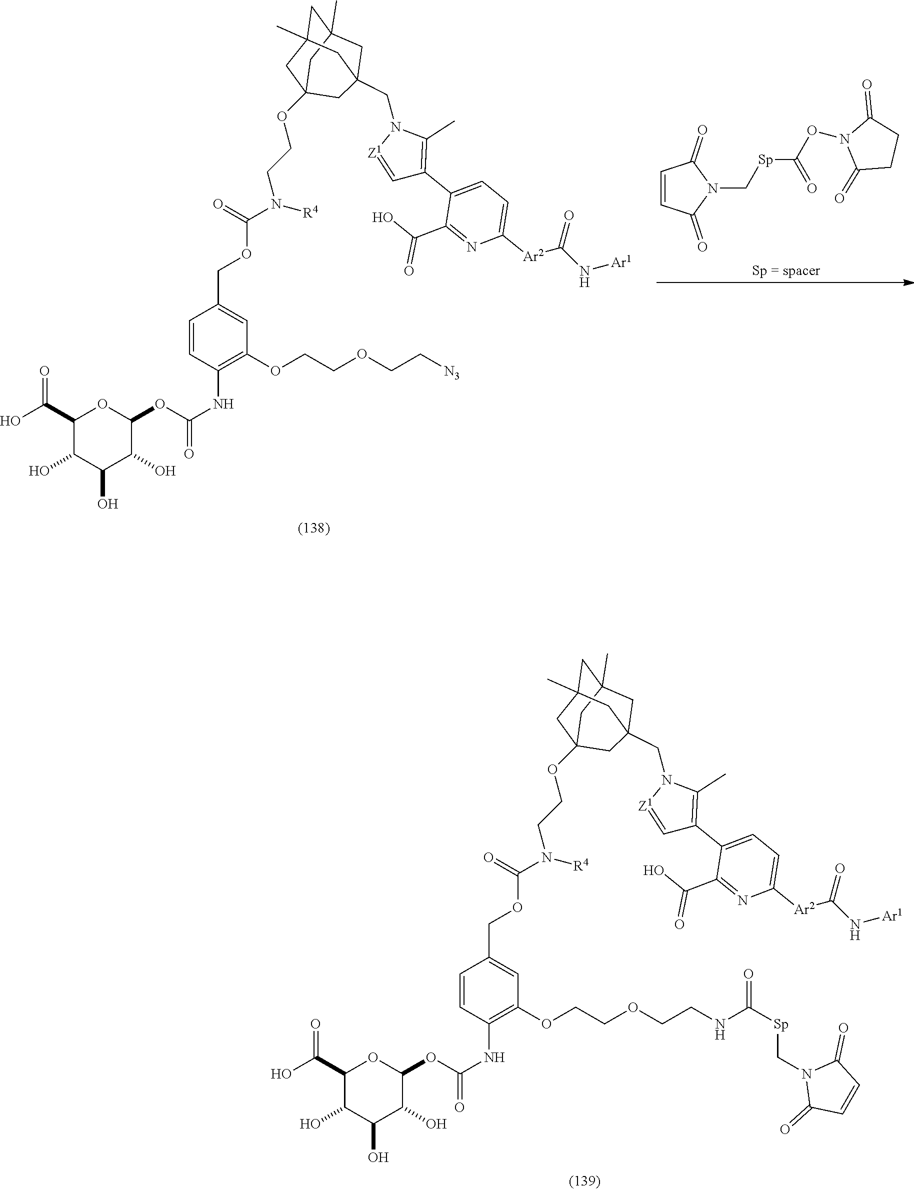

Scheme 19 describes the synthesis of carbamate glucuronide intermediates and synthons. 2-Amino-5-(hydroxymethyl)phenol (130) can be treated with sodium hydride and then reacted with 2-(2-azidoethoxy)ethyl 4-methylbenzenesulfonate (131) to provide (4-amino-3-(2-(2-azidoethoxy)ethoxy)phenyl)methanol (132). The reaction is typically performed at an elevated temperature in a solvent such as, but not limited to N,N-dimethylformamide. 2-(2-(2-Azidoethoxy)ethoxy)-4-(((tert-butyldimethylsilyl)oxy)methyl)aniline (133) can be prepared by reacting (4-amino-3-(2-(2-azidoethoxy)ethoxy)phenyl)methanol (132) with tert-butyldimethylchlorosilane in the presence of imidazole. The reaction is typically performed at ambient temperature in a solvent such as, but not limited to tetrahydrofuran. 2-(2-(2-Azidoethoxy)ethoxy)-4-(((tert-butyldimethylsilyl)oxy)methyl)aniline (133) can be treated with phosgene, in the presence of a base such as but not limited to triethylamine, followed by reaction with (3R,4S,5S,6S)-2-hydroxy-6-(methoxycarbonyl)tetrahydro-2H-pyran-3,4,5-triyl triacetate (134) in the presence of a base such as but not limited to triethylamine, to provide 2S,3R,4S,5S,6S)-2-(((2-(2-(2-azidoethoxy)ethoxy)-4-(((tert-butyldimethylsilyl)oxy)methyl)phenyl)carbamoyl)oxy)-6-(methoxycarbonyl)tetrahydro-2H-pyran-3,4,5-triyl triacetate (135). The reaction is typically performed in a solvent such as, but not limited to, toluene, and the additions are typically performed at low temperature, before warming up to ambient temperature after the phosgene addition and heating at an elevated temperature after the (3R,4S,5S,6S)-2-hydroxy-6-(methoxycarbonyl)tetrahydro-2H-pyran-3,4,5-triyl triacetate (134) addition. (2S,3R,4S,5S,6S)-2-(((2-(2-(2-Azidoethoxy)ethoxy)-4-(hydroxymethyl)phenyl)carbamoyl)oxy)-6-(methoxycarbonyl)tetrahydro-2H-pyran-3,4,5-triyl triacetate (136) can be prepared by reacting 2S,3R,4S,5S,6S)-2-(((2-(2-(2-azidoethoxy)ethoxy)-4-(((tert-butyldimethylsilyl)oxy)methyl)phenyl)carbamoyl)oxy)-6-(methoxycarbonyl)tetrahydro-2H-pyran-3,4,5-triyl triacetate (135) with p-toluenesulfonic acid monohydrate. The reaction is typically performed at ambient temperature in a solvent such as, but not limited to methanol. (2S,3R,4S,5S,6S)-2-(((2-(2-(2-Azidoethoxy)ethoxy)-4-(hydroxymethyl)phenyl)carbamoyl)oxy)-6-(methoxycarbonyl)tetrahydro-2H-pyran-3,4,5-triyl triacetate (136) can be reacted with bis(4-nitrophenyl)carbonate in the presence of a base such as, but not limited to, N,N-diisopropylethylamine, to provide (2S,3R,4S,5S,6S)-2-(((2-(2-(2-azidoethoxy)ethoxy)-4-((((4-nitrophenoxy)carbonyl)oxy)methyl)phenyl)carbamoyl)oxy)-6-(methoxycarbonyl)tetrahydro-2H-pyran-3,4,5-triyl triacetate (137). The reaction is typically performed at ambient temperature in a solvent such as, but not limited to, N,N-dimethylformamide. (2S,3R,4S,5S,6S)-2-(((2-(2-(2-Azidoethoxy)ethoxy)-4-((((4-nitrophenoxy)carbonyl)oxy)methyl)phenyl)carbamoyl)oxy)-6-(methoxycarbonyl)tetrahydro-2H-pyran-3,4,5-triyl triacetate (137) can be reacted with compound in the presence of a base such as, but not limited to, N,N-diisopropylethylamine, followed by treatment with aqueous lithium hydroxide, to provide compound (138). The first step is typically conducted at ambient temperature in a solvent such as, but not limited to N,N-dimethylformamide, and the second step is typically conducted at low temperature in a solvent such as but not limited to methanol. Compound (138) can be treated with tris(2-carboxyethyl))phosphine hydrochloride, followed by reaction with compound (84) in the presence of a base such as, but not limited to, N,N-diisopropylethylamine, to provide compound (139). The reaction with tris(2-carboxyethyl))phosphine hydrochloride is typically performed at ambient temperature in a solvent such as, but not limited to, tetrahydrofuran, water, or mixtures thereof, and the reaction with N-succinimidyl 6-maleimidohexanoate is typically performed at ambient temperature in a solvent such as, but not limited to, N,N-dimethylformamide.

5.2.8. Synthesis of Compound (149)

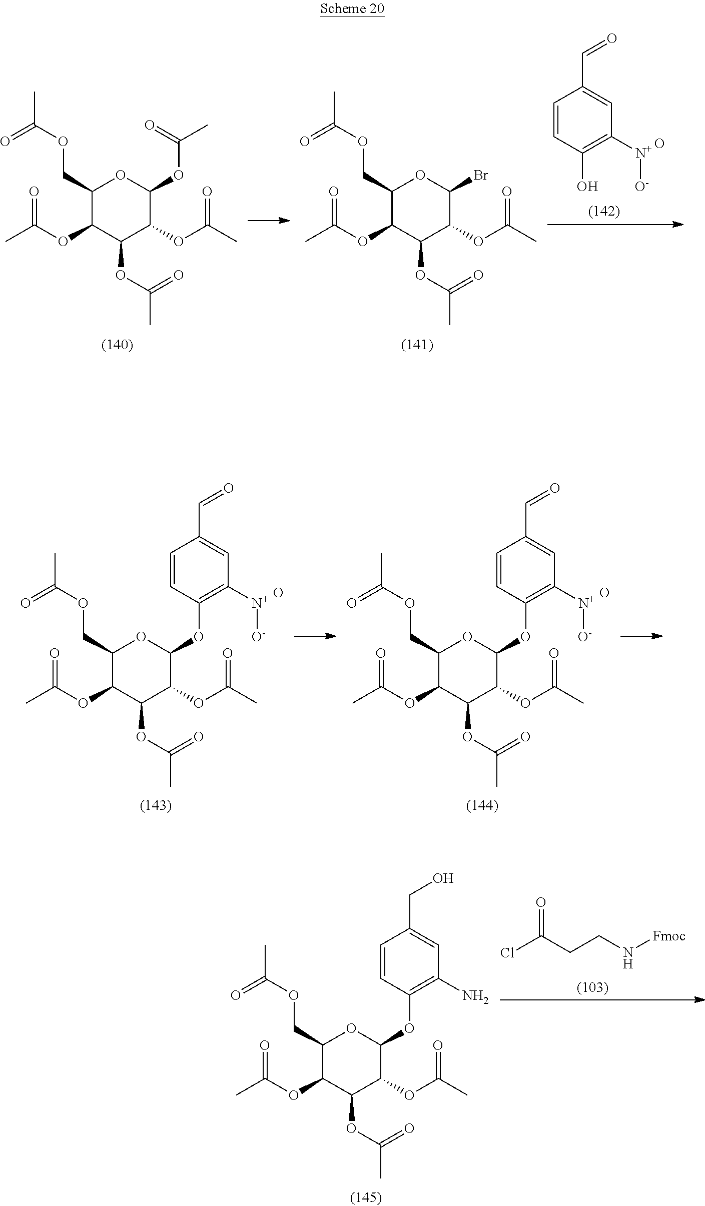

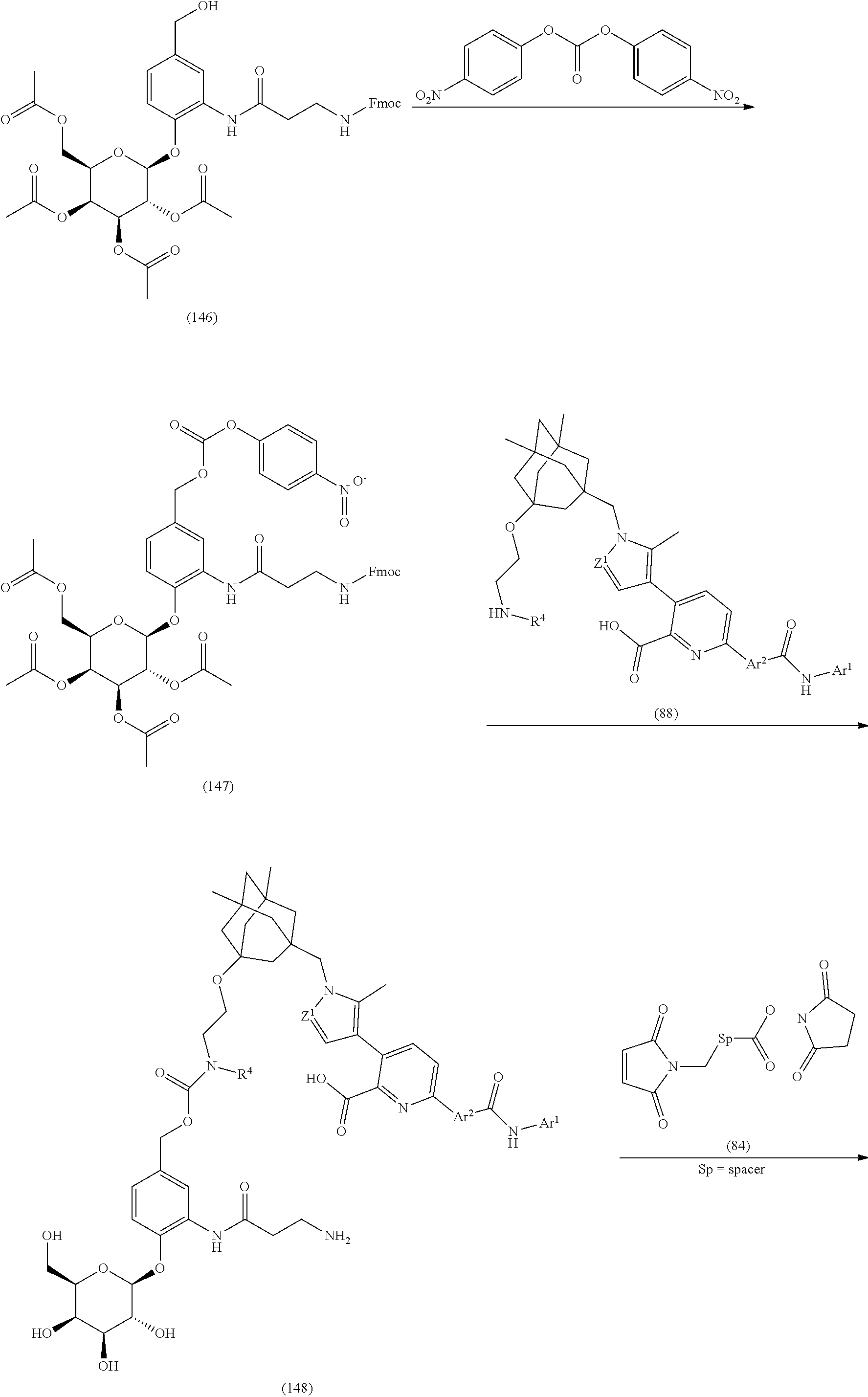

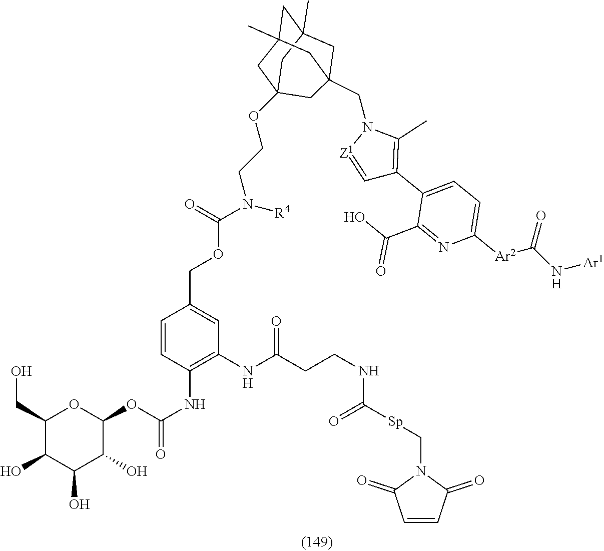

Scheme 20 describes the synthesis of galactoside linker intermediates and synthons. (2S,3R,4S,5S,6R)-6-(Acetoxymethyl)tetrahydro-2H-pyran-2,3,4,5-tetrayl tetraacetate (140) can be treated with HBr in acetic acid to provide (2R,3S,4S,5R,6S)-2-(acetoxymethyl)-6-bromotetrahydro-2H-pyran-3,4,5-triyl triacetate (141). The reaction is typically performed at ambient temperature under a nitrogen atmosphere. (2R,3S,4S,5R,6S)-2-(Acetoxymethyl)-6-(4-formyl-2-nitrophenoxy)tetrahydro-2H-pyran-3,4,5-triyl triacetate (143) can be prepared by treating (2R,3S,4S,5R,6S)-2-(acetoxymethyl)-6-bromotetrahydro-2H-pyran-3,4,5-triyl triacetate (141) with silver(I) oxide in the presence of 4-hydroxy-3-nitrobenzaldehyde (142). The reaction is typically performed at ambient temperature in a solvent such as, but not limited to, acetonitrile. (2R,3S,4S,5R,6S)-2-(Acetoxymethyl)-6-(4-formyl-2-nitrophenoxy)tetrahydro-2H-pyran-3,4,5-triyl triacetate (143) can be treated with sodium borohydride to provide (2R,3S,4S,5R,6S)-2-(acetoxymethyl)-6-(4-(hydroxymethyl)-2-nitrophenoxy)tetrahydro-2H-pyran-3,4,5-triyl triacetate (144). The reaction is typically performed at low temperature in a solvent such as but not limited to tetrahydrofuran, methanol, or mixtures thereof. (2R,3S,4S,5R,6S)-2-(Acetoxymethyl)-6-(2-amino-4-(hydroxymethyl)phenoxy)tetrahydro-2H-pyran-3,4,5-triyl triacetate (145) can be prepared by treating (2R,3S,4S,5R,6S)-2-(acetoxymethyl)-6-(4-(hydroxymethyl)-2-nitrophenoxy)tetrahydro-2H-pyran-3,4,5-triyl triacetate (144) with zinc in the presence of hydrochloric acid. The reaction is typically performed at low temperature, under a nitrogen atmosphere, in a solvent such as, but not limited to, tetrahydrofuran. (2S,3R,4S,5S,6R)-2-(2-(3-((((9H-Fluoren-9-yl)methoxy)carbonyl)amino)propanamido)-4-(hydroxymethyl)phenoxy)-6-(acetoxymethyl)tetrahydro-2H-pyran-3,4,5-triyl triacetate (146) can be prepared by reacting (2R,3S,4S,5R,6S)-2-(acetoxymethyl)-6-(2-amino-4-(hydroxymethyl)phenoxy)tetrahydro-2H-pyran-3,4,5-triyl triacetate (145) with (9H-fluoren-9-yl)methyl (3-chloro-3-oxopropyl)carbamate (103) in the presence of a base such as, but not limited to, N,N-diisopropylethylamine. The reaction is typically performed at low temperature, in a solvent such as, but not limited to, dichloromethane. (2S,3R,4S,5S,6R)-2-(2-(3-((((9H-Fluoren-9-yl)methoxy)carbonyl)amino)propanamido)-4-(hydroxymethyl)phenoxy)-6-(acetoxymethyl)tetrahydro-2H-pyran-3,4,5-triyl triacetate (146) can be reacted with bis(4-nitrophenyl)carbonate in the presence of a base such as, but not limited to, N,N-diisopropylethylamine, to provide (2S,3R,4S,5S,6R)-2-(2-(3-((((9H-fluoren-9-yl)methoxy)carbonyl)amino)propanamido)-4-((((4-nitrophenoxy)carbonyl)oxy)methyl)phenoxy)-6-(acetoxymethyl)tetrahydro-2H-pyran-3,4,5-triyl triacetate (147). The reaction is typically performed at low temperature, in a solvent such as, but not limited to, N,N-dimethylformamide. (2S,3R,4S,5S,6R)-2-(2-(3-((((9H-Fluoren-9-yl)methoxy)carbonyl)amino)propanamido)-4-((((4-nitrophenoxy)carbonyl)oxy)methyl)phenoxy)-6-(acetoxymethyl)tetrahydro-2H-pyran-3,4,5-triyl triacetate (147) can be reacted with compound (88) in the presence of a base such as, but not limited to N,N-diisopropylethylamine, followed by treatment with lithium hydroxide, to provide compound (148). The first step is typically performed at low temperature, in a solvent such as, but not limited to, N,N-dimethylformamide, and the second step is typically performed at ambient temperature, in a solvent such as, but not limited to, methanol. Compound (148) can be treated with compound (84), wherein Sp is a spacer, in the presence of a base, such as, but not limited to N,N-diisopropylethylamine, to provide compound (149). The reaction is typically performed at ambient temperature, in a solvent such as, but not limited to, N,N-dimethylformamide.

### III.6 General Methods for Synthesizing Anti-CD98 ADCs

The present invention also discloses a process to prepare an anti-CD98 ADC according to structural formula (I):

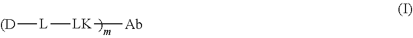

wherein D, L, LK, Ab and m are as defined in the Detailed Description section. The process comprises:

treating an antibody in an aqueous solution with an effective amount of a disulfide reducing agent at 30-40° C. for at least 15 minutes, and then cooling the antibody solution to 20-27° C.;

adding to the reduced antibody solution a solution of water/dimethyl sulfoxide comprising a synthon selected from the group of 2.1 to 2.31 and 2.34 to 2.72 (Table 5);

adjusting the pH of the solution to a pH of 7.5 to 8.5; and

allowing the reaction to run for 48 to 80 hours to form the ADC;

wherein the mass is shifted by 18±2 amu for each hydrolysis of a succinimide to a succinamide as measured by electron spray mass spectrometry; and

wherein the ADC is optionally purified by hydrophobic interaction chromatography.

In certain embodiments, Ab is a CD98 antibody, wherein the CD98 antibody comprises the heavy and light chain CDRs of huAb102, huAb014, huAb105, or huAb110;

The present invention is also directed to an anti-CD98 ADC prepared by the above-described process.

In one embodiment, the anti-CD98 ADC disclosed in the present application is formed by contacting an antibody that binds an hCD98 cell surface receptor or tumor associated antigen expressed on a tumor cell with a drug-linker synthon under conditions in which the drug-linker synthon covalently links to the antibody through a maleimide moiety as shown in formula (lid) or (IIe).

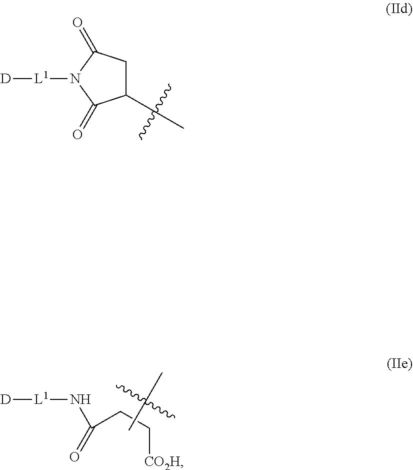

wherein D is the Bcl-xL inhibitor drug according to structural formula (IIa) or (IIb) as described above and L¹ is the portion of the linker not formed from the maleimide upon attachment of the synthon to the antibody; and wherein the drug-linker synthon is selected from the group consisting of synthon examples 2.1 to 2.31 and 2.34 to 2.72 (Table 5), or a pharmaceutically acceptable salt thereof.

In certain embodiments, the contacting step is carried out under conditions such that the anti-CD98 ADC has a DAR of 2, 3 or 4.

### III.B. Anti-CP98 APCs: Other Exemplary Drugs for Conjugation

Anti-CD98 antibodies may be used in ADCs to target one or more drug(s) to a cell of interest e.g., a cancer cell expressing CD98. The anti-CD98 ADCs of the invention provide a targeted therapy that may, for example, reduce the side effects often seen with anti-cancer therapies, as the one or more drug(s) is delivered to a specific cell.

### Auristatins

Anti-CD98 antibodies of the invention, e.g., the huAb102, huAb104, huAb105, or huAb110 antibody, may be conjugated to at least one auristatin. Auristatins represent a group of dolastatin analogs that have generally been shown to possess anticancer activity by interfering with microtubule dynamics and GTP hydrolysis, thereby inhibiting cellular division. For example, auristatin E (U.S. Pat. No. 5,635,483) is a synthetic analogue of the marine natural product dolastatin 10, a compound that inhibits tubulin polymerization by binding to the same site on tubulin as the anticancer drug vincristine (G. R. Pettit, Prog. Chem. Org. Nat. Prod, 70: 1-79 (1997)). Dolastatin 10, auristatin PE, and auristatin E are linear peptides having four amino acids, three of which are unique to the dolastatin class of compounds. Exemplary embodiments of the auristatin subclass of mitotic inhibitors include, but are not limited to, monomethyl auristatin D (MMAD or auristatin D derivative), monomethyl auristatin E (MMAE or auristatin E derivative), monomethyl auristatin F (MMAF or auristatin F derivative), auristatin F phenylenediamine (AFP), auristatin EB (AEB), auristatin EFP (AEFP), and 5-benzoylvaleric acid-AE ester (AEVB). The synthesis and structure of auristatin derivatives are described in U.S. Patent Application Publication Nos. 2003-0083263, 2005-0238649 and 2005-009751; International Patent Publication No. WO 04/010957. International Patent Publication No. WO 02/088172, and U.S. Pat. Nos. 6,323,315; 6.239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, each of which is incorporated by reference herein.

In one embodiment anti-CD98 antibodies of the invention, e.g., huAb102, huAb104, huAb105, or huAb110, are conjugated to at least one MMAE (mono-methyl auristatin E). Monomethyl auristatin E (MMAE, vedotin) inhibits cell division by blocking the polymerization of tubulin. However, due to its super toxicity, auristatin E cannot be used as a drug itself. Auristatin E can be linked to a monoclonal antibody (mAb) that recognizes a specific marker expression in cancer cells and directs MMAE to the cancer cells. In one embodiment the linker linking MMAE to the anti-CD98 antibody is stable in extracellular fluid (i.e., the medium or environment that is external to cells), but is cleaved by cathepsin once the ADC has bound to the specific cancer cell antigen and entered the cancer cell, thus releasing the toxic MMAE and activating the potent anti-mitotic mechanism.

In one embodiment an anti-CD98 antibody described herein, e.g., huAb102, huAb104, huAb108, or huAb110, is conjugated to at least one MMAE (monomethylauristatin F). Monomethyl auristatin F (MMAF) inhibits cell division by blocking the polymerization of tubulin. It has a charged C-terminal phenylalanine residue that attenuates its cytotoxic activity compared to its uncharged counterpart MMAE. However, due to its super toxicity, auristatin F cannot be used as a drug itself, but can be linked to a monoclonal antibody (mAb) that directs it to the cancer cells. In one embodiment, the linker to the anti-CD98 antibody is stable in extracellular fluid, but is cleaved by cathepsin once the conjugate has entered a tumor cell, thus activating the anti-mitotic mechanism.

The structures of MMAF and MMAE are provided below.

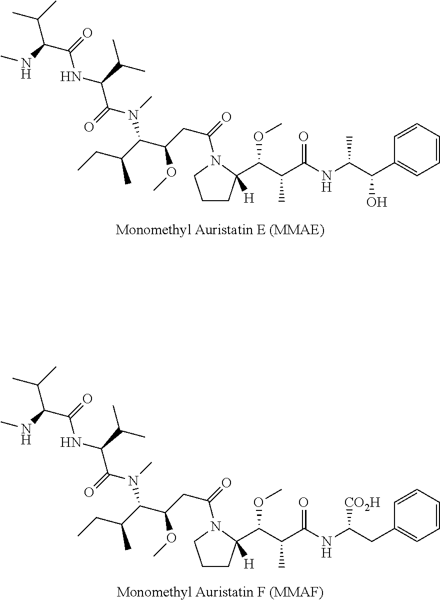 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C ; CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C]

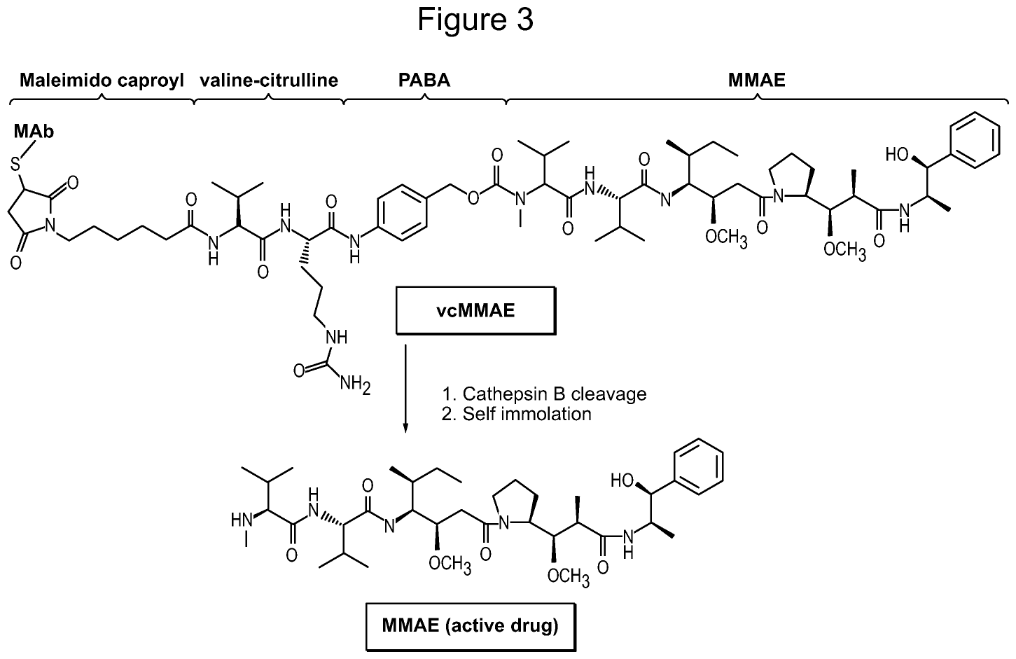

An example of huAb102, huAb104, huAb108, or huAb110-vcMMAE is also provided in FIG. 3. Notably, FIG. 3 describes a situation where the antibody (e.g., huAb102, huAb104, huAb108, or huAb110) is coupled to a single drug and, therefore, has a DAR of 1. In certain embodiments, the ADC will have a DAR of 2 to 8, or, alternatively, 2 to 4.

### Other Drugs for Conjugation

Examples of drugs that may be used in ADCs, i.e., drugs that may be conjugated to the anti-CD98 antibodies of the invention, are provided below, and include mitotic inhibitors, antitumor antibiotics, immunomodulating agents, gene therapy vectors, alkylating agents, antiangiogenic agents, antimetabolites, boron-containing agents, chemoprotective agents, hormone agents, glucocorticoids, photoactive therapeutic agents, oligonucleotides, radioactive isotopes, radiosensitizers, topoisomerase inhibitors, kinase inhibitors, and combinations thereof.

### 1. Mitotic Inhibitors

In one aspect anti-CD98 antibodies may be conjugated to one or more mitotic inhibitors) to form an ADC for the treatment of cancer. The term “mitotic inhibitor”, as used herein, refers to a cytotoxic and/or therapeutic agent that blocks mitosis or cell division, a biological process particularly important to cancer cells. A mitotic inhibitor disrupts microtubules such that cell division is prevented, often by effecting microtubule polymerization (e.g., inhibiting microtubule polymerization) or microtubule depolymerization (e.g., stabilizing the microtubule cytoskeleton against depolymerization). Thus, in one embodiment, an anti-CD98 antibody of the invention is conjugated to one or more mitotic inhibitor(s) that disrupts microtubule formation by inhibiting tubulin polymerization. In another embodiment an anti-CD98 antibody of the invention is conjugated to one or more mitotic inhibitor(s) that stabilizes the microtubule cytoskeleton from depolymerization. In one embodiment, the mitotic inhibitor used in the ADCs of the invention is Ixempra (ixabepilone). Examples of mitotic inhibitors that may be used in the anti-CD98 ADCs of the invention are provided below. Included in the genus of mitotic inhibitors are auristatins, described above.

a. Dolastatins

The anti-CD98 antibodies of the invention may be conjugated to at least one dolastatin to form an ADC. Dolastatins are short peptidic compounds isolated from the Indian Ocean sea hare Dolabella auricularia (see Pettit et al., J. Am. Chem. Soc., 1976, 98, 4677). Examples of dolastatins include dolastatin 10 and dolastatin 15. Dolastatin 15, a seven-subunit depsipeptide derived from Dolabella auricularia, and is a potent antimitotic agent structurally related to the antitubulin agent dolastatin 10, a five-subunit peptide obtained from the same organism. Thus, in one embodiment, the anti-CD98 ADC of the invention comprises an anti-CD98 antibody, as described herein, and at least one dolastatin. Auristatins, described above, are synthetic derivatives of dolastatin 10.

b. Maytansinoids

The anti-CD98 antibodies of the invention may be conjugated to at least one maytansinoid to form an ADC. Maytansinoids are potent antitumor agents that were originally isolated from members of the higher plant families Celastraceae, Rhamnaceae, and Euphorbiaceae, as well as some species of mosses (Kupchan et al, J. Am. Chem. Soc. 94:1354-1356 [1972]; Wani et al, J. Chem. Soc. Chem. Commun. 390: [1973]; Powell et al, J. Nat. Prod. 46:660-666 [1983]; Sakai et al. J. Nat. Prod. 51:845-850 [1988]; and Suwanborirux et al, Experientia 46:117-120 [1990]). Evidence suggests that maytansinoids inhibit mitosis by inhibiting polymerization of the microtubule protein tubulin, thereby preventing formation of microtubules (see, e.g., U.S. Pat. No. 6,441,163 and Remillard et al., Science, 189, 1002-1005 (1975)). Maytansinoids have been shown to inhibit tumor cell growth in vitro using cell culture models, and in vivo using laboratory animal systems. Moreover, the cytotoxicity of maytansinoids is 1,000-fold greater than conventional chemotherapeutic agents, such as, for example, methotrexate, daunorubicin, and vincristine (see, e.g., U.S. Pat. No. 5,208,020).

Maytansinoids to include maytansine, maytansinol, C-3 esters of maytansinol, and other maytansinol analogues and derivatives (see, e.g., U.S. Pat. Nos. 5,208,020 and 6,441,163, each of which is incorporated by reference herein). C-3 esters of maytansinol can be naturally occurring or synthetically derived. Moreover, both naturally occurring and synthetic C-3 maytansinol esters can be classified as a C-3 ester with simple carboxylic acids, or a C-3 ester with derivatives of N-methyl-L-alanine, the latter being more cytotoxic than the former. Synthetic maytansinoid analogues are described in, for example, Kupchan et al, J. Med. Chem., 21, 31-37 (1978).

Suitable maytansinoids for use in ADCs of the invention can be isolated from natural sources, synthetically produced, or semi-synthetically produced. Moreover, the maytansinoid can be modified in any suitable manner, so long as sufficient cytotoxicity is preserved in the ultimate conjugate molecule. In this regard, maytansinoids lack suitable functional groups to which antibodies can be linked. A linking moiety desirably is utilized to link the maytansinoid to the antibody to form the conjugate, and is described in more detail in the linker section below. The structure of an exemplary maytansinoid, mertansine (DM1), is provided below.

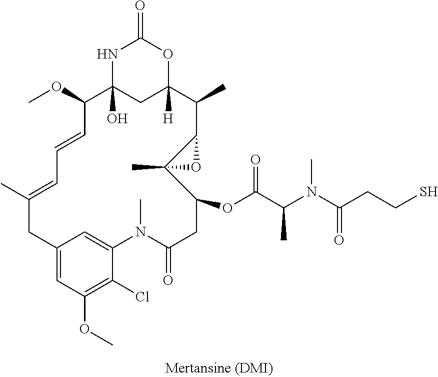

Representative examples of maytansinoids include, but are not limited, to DM1 (N^{2′}-deacetyl-N^{2′}-(3-mercapto-1-oxopropyl)-maytansine; also referred to as mertansine, drug maytansinoid 1; ImmunoGen, Inc.; see also Chari et al. (1992) Cancer Res 52:127), DM2. DM3 (N^{2′}-deacetyl-N^{2′}-(4-mercapto-1-oxopentyl)-maytansine), DM4 (4-methyl-4-mercapto-1-oxopentyl)-maytansine), and maytansinol (a synthetic maytansinoid analog). Other examples of maytansinoids are described in U.S. Pat. No. 8,142,784, incorporated by reference herein.

Ansamitocins are a group of maytansinoid antibiotics that have been isolated from various bacterial sources. These compounds have potent antitumor activities. Representative examples include, but are not limited to ansamitocin P1, ansamitocin P2, ansamitocin P3, and ansamitocin P4.

In one embodiment of the invention, an anti-CD98 antibody is conjugated to at least one DM1. In one embodiment, an anti-CD98 antibody is conjugated to at least one DM2. In one embodiment, an anti-CD98 antibody is conjugated to at least one DM3. In one embodiment, an anti-CD98 antibody is conjugated to at least one DM4.

d. Plant Alkaloids

The anti-CD98 antibodies of the invention may be conjugated to at least one plant alkaloid, e.g., a taxane or vinca alkaloid. Plant alkaloids are chemotherapy treatments derived made from certain types of plants. The vinca alkaloids are made from the periwinkle plant (catharanthus rosea), whereas the taxanes are made from the bark of the Pacific Yew tree (taxus). Both the vinca alkaloids and taxanes are also known as antimicrotubule agents, and are described in more detail below.

### Taxanes

Anti-CD98 antibodies described herein may be conjugated to at least one taxane. The term “taxane” as used herein refers to the class of antineoplastic agents having a mechanism of microtubule action and having a structure that includes the taxane ring structure and a stereospecific side chain that is required for cytostatic activity. Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869, each of which is incorporated by reference herein. Taxane compounds have also previously been described in U.S. Pat. Nos. 5,641,803, 5,665,671, 5,380,751, 5,728,687, 5,415,869, 5,407,683, 5,399,363, 5,424,073, 5,157,049, 5,773,464, 5,821,263, 5,840,929, 4,814,470, 5,438,072, 5,403,858, 4,960,790, 5,433,364, 4,942,184, 5,362,831, 5,705,503, and 5,278,324, all of which are expressly incorporated by reference. Further examples of taxanes include, but are not limited to, docetaxel (Taxotere; Sanofi Aventis), paclitaxel (Abraxane or Taxol; Abraxis Oncology), carbazitaxel, tesctaxel, opaxio, larotaxel, taxoprexin, BMS-184476, hongdoushan A, hongdoushan B, and hongdoushan C, and nanoparticle paclitaxel (ABI-007/Abraxene; Abraxis Bioscience).

In one embodiment, the anti-CD98 antibody of the invention is conjugated to at least one docetaxel molecule. In one embodiment, the anti-CD98 antibody of the invention is conjugated to at least one paclitaxel molecule.

### Vinca Alkaloids

In one embodiment, the anti-CD98 antibody is conjugated to at least one vinca alkaloid. Vinca alkaloids are a class of cell-cycle-specific drugs that work by inhibiting the ability of cancer cells to divide by acting upon tubulin and preventing the formation of microtubules. Examples of vinca alkaloids that may be used in the ADCs of the invention include, but are not limited to, vindesine sulfate, vincristine, vinblastine, and vinorelbine.

### 2. Antitumor Antibiotics

Anti-CD98 antibodies of the invention may be conjugated to one or more antitumor antibiotic(s) for the treatment of cancer. As used herein, the term “antitumor antibiotic” means an anti neoplastic drug that blocks cell growth by interfering with DNA and is made from a microorganism. Often, antitumor antibiotics either break up DNA strands or slow down or stop DNA synthesis. Examples of antitumor antibiotics that may be included in the anti-CD98 ADCs of the invention include, but are not limited to, actinomycines (e.g., pyrrolo[2,1-c][1,4]benzodiazepines), anthracyclines, calicheamicins, and duocarmycins, described in more detail below.

a. Actinomycins

The anti-CD98 antibodies of the invention may be conjugated to at least one actinomycin. Actinomycins are a subclass of antitumor antibiotics isolated from bacteria of the genus Streptomyces. Representative examples actinomycins include, but are not limited to, actinomycin D (Cosmegen [also known as actinomycin, dactinomycin, actinomycin IV, actinomycin C1]. Lundbeck, Inc.), anthramycin, chicamycin A, DC-81, mazethramycin, neothramvein A, neothramycin B, porothramvein, prothracarcin B, SG2285, sibanomicin, sibiromycin, and tomaymycin. In one embodiment, the anti-CD98 antibody of the invention is conjugated to at least one pyrrolobenzodiazepine (PBD). Examples of PBDs include, but are not limited to, anthramycin, chicamycin A, DC-81, mazethramycin, neothramycin A, neothramycin B, porothramycin, prothracarcin B, SG2000 (SJG-136), SG2202 (ZC-207), SG2285 (ZC-423), sibanomicin, sibiromycin and tomaymycin. Thus, in one embodiment, anti-CD98 antibodies of the invention are conjugated to at least one actinomycin, e.g., actinomycin D, or at least one PBD, e.g., a pyrrolobenzodiazepine (PBD) dimer.

The structures of PBDs can be found, for example, in U.S. Patent Application Pub. Nos. 2013/0028917 and 2013/0028919, and in WO 2011/130598 A1, each of which are incorporated herein by reference in their entirety. The generic structure of a PBD is provided below.

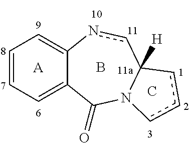

PBDs differ in the number, type and position of substituents, in both their aromatic A rings and pyrrolo C rings, and in the degree of saturation of the C ring. In the B-ring, there is generally an imine (N═C), a carbinolamine (NH—CH(OH)), or a carbinolamine methyl ether (NH—CH(OMe)) at the N10-C11 position which is the electrophilic center responsible for alkylating DNA. All of the known natural products have an (S)-configuration at the chiral C11α position which provides them with a right-handed twist when viewed from the C ring towards the A ring. The PBD examples provided herein may be conjugated to the anti-CD98 antibodies of the invention. Further examples of PBDs which may be conjugated to the anti-CD98 antibodies of the invention can be found, for example, in U.S. Patent Application Publication Nos. 2013/0028917 A1 and 2013/0028919 A1, in U.S. Pat. No. 7,741,319 B2, and in WO 2011/130598 A1 and WO 2006/111759 A1, each of which are incorporated herein by reference in their entirety.

A representative PBD dimer having the following formula XXX may be conjugated to the anti-CD98 antibodies of the invention:

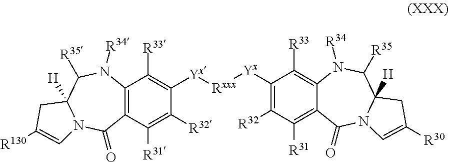

wherein:

R³⁰ is of formula XXXI:

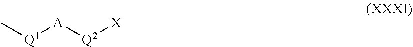

where A is a C₅₋₇ aryl group. X is a group conjugated to the Linker unit selected from the group consisting of —O—, —S—, —C(o)O—, —C(O)—, —NH(C═O)—, and —N(R^{N})—, wherein R^{N} is selected from the group consisting of H, C₁₋₄ alkyl and (C₂H₄O)ₘCH₃, where s is 1 to 3, and either:

(i) Q¹ is a single bond, and Q² is selected from the group consisting of a single bond and —Z—(CH₂)ₙ—, where Z is selected from the group consisting of a single bond. O, S and NH and n is from 1 to 3; or

(ii) Q¹ is —CH═CH—, and Q² is a single bond;

R¹³⁰ is a C₅₋₁₀ aryl group, optically substituted by one or more substituents selected from the group consisting of halo, nitro, cyano, C₁₋₁₂ alkoxy, C₃₋₂₀ heterocycloalkoxy, C₅₋₂₀ aryloxy, heteroaryloxy, alkylalkoxy, alkylalkoxy, arylalkoxy, alkylaryloxy, heteroarylalkoxy, alkylheteroaryloxy, C₁₋₇ alkyl, C₃₋₇ heterocyclyl and bis-oxy-C₁₋₃ alkylene;

R³¹ and R³³ are independently selected from the group consisting of H, R^{x}, OH, OR^{x}, SH, SR^{x}, NH₂, NHR^{x}, NR^{x}R^{xx′}, nitro, Me₃Sn and halo;

where R and R′ are independently selected from the group consisting of optionally substituted C₁₋₁₂ alkyl, C₃₋₂₀ heterocyclyl and C₅₋₂₀ aryl groups;

R³² is selected from the group consisting of H, R^{x}, OH, OR^{x}, SH, SR^{x}, NH₂, NHR^{x}, NHR^{x}R^{xx}, nitro, Me₃Sn and halo;

either:

(a) R³⁴ is H, and R¹¹ is OH, OR^{xA}, where R^{xA} is C₁₋₄ alkyl;

(b) R³⁴ and R³⁵ form a nitrogen-carbon double bond between the nitrogen and carbon atoms to which they are bound; or

(c) R³⁴ is H and R³³ is SO_{z}M, where z is 2 or 3;

R^{xxx} is a C₃₋₁₂ alkylene group, which chain may be interrupted by one or more heteroatoms, selected from the group consisting of O, S, NH, and an aromatic ring;

Y^{x} and Y^{x′} are is selected from the group consisting of O, S, and NH;

R^{31′}, R^{32′}, R^{33′} are selected from the same groups as R³¹, R³² and R³³ respectively and R^{34′} and R³⁵ are the same as R³⁴ and R³⁵, and each M is a monovalent pharmaceutically acceptable cation or both M groups together are a divalent pharmaceutically acceptable cation.

C₁₋₁₂ alkyl: The term “C₁₋₁₂ alkyl” as used herein, pertains to a monovalent moiety obtained by removing a hydrogen atom from a carbon atom of a hydrocarbon compound having from 1 to 12 carbon atoms, which may be aliphatic or alicyclic, and which may be saturated or unsaturated (e.g. partially unsaturated, fully unsaturated). Thus, the term “alkyl” includes the sub-classes alkenyl, alkynyl, cycloalkyl, etc., discussed below.

Examples of saturated alkyl groups include, but are not limited to, methyl (C₁), ethyl (C₂), propyl (C₃), butyl (C₄), pentyl (C₅), hexyl (C₆) and heptyl (C₇).

Examples of saturated linear alkyl groups include, but are not limited to, methyl (C₁), ethyl (C₂), n-propyl (C₃), n-butyl (C₄), n-pentyl (amyl) (C₅), n-hexyl (C₆) and n-heptyl (C₇).

Examples of saturated branched alkyl groups include iso-propyl (C₃), iso-butyl (C₄), sec-butyl (C₄), tert-butyl (C₄), iso-pentyl (C₅), and neo-pentyl (C₅).

C₃₋₂₀ heterocyclyl: The term “C₃₋₂₀ heterocyclyl” as used herein, pertains to a monovalent moiety obtained by removing a hydrogen atom from a ring atom of a heterocyclic compound, which moiety has from 3 to 20 ring atoms, of which from 1 to 10 are ring heteroatoms. Preferably, each ring has from 3 to 7 ring atoms, of which from 1 to 4 are ring heteroatoms.

In this context, the prefixes (e.g. C₃₋₂₀, C₃₋₇, C₅₋₆, etc.) denote the number of ring atoms, or range of number of ring atoms, whether carbon atoms or heteroatoms. For example, the term “C₅₋₆heterocyclyl”, as used herein, pertains to a heterocyclyl group having 5 or 6 ring atoms.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇); O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇); S₁: thiirane (C₃), thietane (C₄), thiolane (tetrahydrothiophene) (C₅), thiane (tetrahydrothiopyran) (C₆), thiepane (C₇); O₂: dioxolane (C₅), dioxane (C₆), and dioxepane (C₇); O₃: trioxane (C₆); N₂: imidazolidine (C₅), pyrazolidine (diazolidine) (C₅), imidazoline (C₅), pyrazoline (dihydropyrazole) (C₅), piperazine (C₆); N₁O₁: tetrahydrooxazole (C₅), dihydrooxazole (C₅), tetrahydroisoxazole (C₅), dihydroisoxazole (C₅), morpholine (C₅), tetrahydrooxazine (C₅), dihydrooxazine (C₆), oxazine (C₆); N1S₁: thiazoline (C₅), thiazolidine (C₅), thiomorpholine (C₆); N₂O₁: oxadiazine (C₆); O₁S₁: oxathiole (C₅) and oxathiane (thioxane) (C₆); and, N₁O₁S₁: oxathiazine (C₆).

Examples of substituted monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

C₅₋₂₀ aryl: The term “C₅₋₂₀ aryl”, as used herein, pertains to a monovalent moiety obtained by removing a hydrogen atom from an aromatic ring atom of an aromatic compound, which moiety has from 3 to 20 ring atoms. Preferably, each ring has from 5 to 7 ring atoms.

In this context, the prefixes (e.g. C₃₋₂₀, C₅₋₇, C₅₋₆, etc.) denote the number of ring atoms, or range of number of ring atoms, whether carbon atoms or heteroatoms. For example, the term “C₅₋₆ aryl” as used herein, pertains to an aryl group having 5 or 6 ring atoms.

In one embodiment, the anti-CD98 antibodies of the invention may be conjugated to a PBD dimer having the following formula XXXIa:

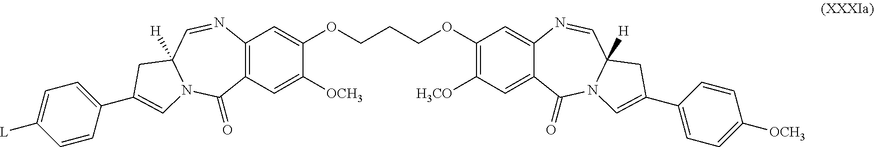

wherein the above structure describes the PBD dimer SG2202 (ZC-207) and is conjugated to the anti-CD98 antibody of the invention via a linker L. SG2202 (ZC-207) is disclosed in, for example, U.S. Patent App. Pub. No. 2007/0173497, which is incorporated herein by reference in its entirety.

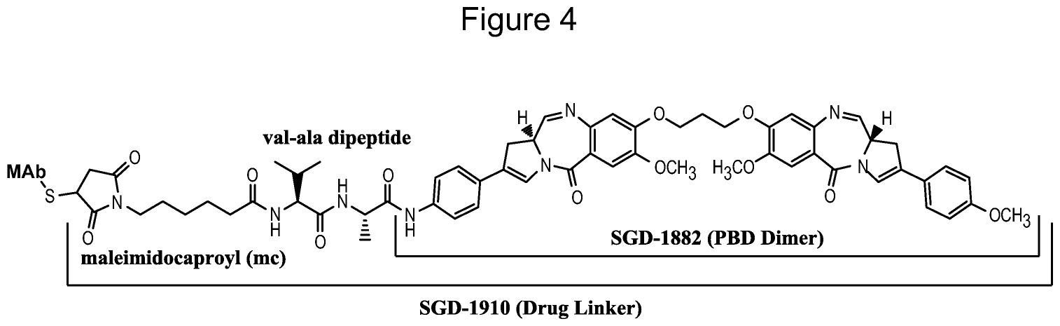

In another embodiment, a PBD dimer, SGD-1882, is conjugated to anti-CD98 antibody of the invention via a drug linker, as depicted in FIG. 4. SGD-1882 is disclosed in Sutherland et al. (2013) Blood 122(8): 1455 and in U.S Patent App. Pub. No. 2013/0028919, which is incorporated herein by reference in its entirety. As described in FIG. 4, the PBD dimer SGD-1882 may be conjugated to an antibody via an mc-val-ala-dipeptide linker (collectively referred to as SGD-1910 in FIG. 4). In a certain embodiment, an anti-CD98 antibody, as disclosed herein, is conjugated to the PBD dimer described in FIG. 4. Thus, in a further embodiment, the invention includes an anti-CD98 antibody, as disclosed herein, conjugated to a PBD dimer via a mc-val-ala-dipeptide linker, as described in FIG. 4. In certain embodiments, the invention includes an anti-CD98 antibody comprising a heavy chain variable region comprising a CDR3 domain comprising the amino acid sequence of SEQ ID NO: 12, a CDR2 domain comprising the amino acid sequence of SEQ ID NO: 11, and a CDR1 domain comprising the amino acid sequence of SEQ ID NO: 10, and a light chain variable region comprising a CDR3 domain comprising the amino acid sequence of SEQ ID NO: 8, a CDR2 domain comprising the amino acid sequence of SEQ ID NO: 7, and a CDR1 domain comprising the amino acid sequence of SEQ ID NO: 6, conjugated to a PBD, including, but not limited to, the PBD dimer described in FIG. 4. In certain embodiments, the invention includes an anti-CD98 antibody-comprising the heavy chain variable region of huAb102, huAbKH, huAb105, or huAb110 as defined by the amino acid sequence set forth in SEQ ID NO: 108, 110, 115, or 118, respectively, and a light chain variable region comprising the amino acid sequence of SEQ ID NO: 107 (huAb102 and huAb04), or SEQ ID NO: 112 (huAb108 and huAb110), wherein the antibody is conjugated to a PBD, such as, but not limited to, the exemplary PBD dimer of FIG. 4.

b. Anthracyclines

Anti-CD98 antibodies of the invention may be conjugated to at least one anthracycline. Anthracyclines are a subclass of antitumor antibiotics isolated from bacteria of the genus Streptomyces. Representative examples include, but are not limited to daunorubicin (Cerubidine, Bedford Laboratories), doxorubicin (Adriamycin, Bedford Laboratories; also referred to as doxorubicin hydrochloride, hydroxydaunorubicin, and Rubex), epirubicin (Ellence, Pfizer), and idarubicin (Idamycin; Pfizer Inc.). Thus, in one embodiment, the anti-CD98 antibody of the invention is conjugated to at least one anthracycline, e.g., doxorubicin.

c. Calicheamicins

The anti-CD98 antibodies of the invention may be conjugated to at least one calicheamicin. Calicheamicins are a family of enediyne antibiotics derived from the soil organism Micromonospora echinospora. Calicheamicins bind the minor groove of DNA and induce double-stranded DNA breaks, resulting in cell death with a 100 fold increase over other chemotherapeutics (Damle et al. (2003) Curr Opin Pharmacol 3:386). Preparation of calicheamicins that may be used as drug conjugates in the invention have been described, sec U.S. Pat. Nos. 5,712,374; 5,714,586; 5,739.116; 5,767,285; 5,770.701; 5.770,710; 5,773.001; and 5.877,2%. Structural analogues of calicheamicin which may be used include, but are not limited to, γᵢ¹, α₂¹, α₃¹, N-acetyl-γ₁¹, PSAG and θ¹₁ (Hinman et al., Cancer Research 53:3336-3342 (1993). Lode et al., Cancer Research 58:2925-2928 (1998) and the aforementioned U.S. Pat. Nos. 5,712,374; 5,714,586; 5,739,116; 5,767,285; 5.770,701; 5,770.710; 5.773,001; and 5,877,296). Thus, in one embodiment, the anti-CD98 antibody of the invention is conjugated to at least one calicheamicin.

d. Duocarmycins

Anti-CD98 antibodies of the invention may be conjugated to at least one duocarmycin. Duocarmycins are a subclass of antitumor antibiotics isolated from bacteria of the genus Streptomyces. (see Nagamura and Saito (1998) Chemistry of Heterocyclic Compounds, Vol. 34, No. 12). Duocarmycins bind to the minor groove of DNA and alkylate the nucleobase adenine at the N3 position (Boger (1993) Pure and Appl Chem 65(6):1123; and Boger and Johnson (1995) PNAS USA 92:3642). Synthetic analogs of duocarmycins include, but are not limited to, adozclcsin, bizelcsin, and carzelesin. Thus, in one embodiment, the anti-CD98 antibody of the invention is conjugated to at least one duocarmycin.

e. Other Antitumor Antibiotics

In addition to the foregoing, additional antitumor antibiotics that may be used in the anti-CD98 ADCs of the invention include bleomycin (Blenoxane, Bristol-Myers Squibb), mitomycin, and plicamycin (also known as mithramycin).

### 3. Immunomodulating Agents

In one aspect, anti-CD98 antibodies of the invention may be conjugated to at least one immunomodulating agent. As used herein, the term “immunomodulating agent” refers to an agent that can stimulate or modify an immune response. In one embodiment, an immunomodulating agent is an immunostimulator that enhances a subject's immune response. In another embodiment, an immunomodulating agent is an immunosuppressant that prevents or decreases a subject's immune response. An immunomodulating agent may modulate myeloid cells (monocytes, macrophages, dendritic cells, megakaryocytes and granulocytes) or lymphoid cells (T cells, B cells and natural killer (NK) cells) and any further differentiated cell thereof. Representative examples include, but are not limited to, bacillus Calmette-Guerin (BCG) and levamisole (Ergamisol). Other examples of immunomodulating agents that may be used in the ADCs of the invention include, but are not limited to, cancer vaccines, cytokines, and immunomodulating gene therapy.

a. Cancer Vaccines

Anti-CD98 antibodies of the invention may be conjugated to a cancer vaccine. As used herein, the term “cancer vaccine” refers to a composition (e.g., a tumor antigen and a cytokine) that elicits a tumor-specific immune response. The response is elicited from the subject's own immune system by administering the cancer vaccine, or, in the case of the instant invention, administering an ADC comprising an anti-CD98 antibody and a cancer vaccine. In preferred embodiments, the immune response results in the eradication of tumor cells in the body (e.g., primary or metastatic tumor cells). The use of cancer vaccines generally involves the administration of a particular antigen or group of antigens that are, for example, present on the surface a particular cancer cell, or present on the surface of a particular infectious agent shown to facilitate cancer formation. In some embodiments, the use of cancer vaccines is for prophylactic purposes, while in other embodiments, the use is for therapeutic purposes. Non-limiting examples of cancer vaccines that may be used in the anti-CD98 ADCs of the invention include, recombinant bivalent human papillomavirus (HPV) vaccine types 16 and 18 vaccine (Cervarix, GlaxoSmithKline), recombinant quadrivalent human papillomavirus (HPV) types 6, 11, 16, and 18 vaccine (Gardasil, Merck & Company), and sipuleucel-T (Provenge, Dendreon). Thus, in one embodiment, the anti-CD98 antibody of the invention is conjugated to at least one cancer vaccine that is either an immunostimulator or is an immunosuppressant.

b. Cytokines

The anti-CD98 antibodies of the invention may be conjugated to at least one cytokine. The term “cytokine” generally refers to proteins released by one cell population which act on another cell as intercellular mediators. Cytokines directly stimulate immune effector cells and stromal cells at the tumor site and enhance tumor cell recognition by cytotoxic effector cells (Lee and Margolin (2011) Cancers 3:3856). Numerous animal tumor model studies have demonstrated that cytokines have broad anti-tumor activity and this has been translated into a number of cytokine-based approaches for cancer therapy (Lee and Margoli, supra). Recent years have seen a number of cytokines, including GM-CSF, IL-7, IL-12, IL-15, IL-18 and IL-21, enter clinical trials for patients with advanced cancer (Lee and Margoli, supra).

Examples of cytokines that may be used in the ADCs of the invention include, but are not limited to, parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor; mullerian-inhibiting substance; mouse gonadotropin-associated peptide; inhibin; activin; vascular endothelial growth factor; integrin; thrombopoietin (TPO); nerve growth factors such as NGF; platelet-growth factor; transforming growth factors (TGFs); insulin-like growth factor-I and -II; erythropoietin (EPO); osteoinductive factors; interferons such as interferon α, β, and γ, colony stimulating factors (CSFs); granulocyte-macrophage-C—SF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-11, IL-12; tumor necrosis factor, and other polypeptide factors including LIF and kit ligand (KL). As used herein, the term cytokine includes proteins from natural sources or from recombinant cell culture and biologically active equivalents of the native sequence cytokines. Thus, in one embodiment, the invention provides an ADC comprising an anti-CD98 antibody described herein and a cytokine.

c. Colony-Stimulating Factors (CSFs)

The anti-CD98 antibodies of the invention may be conjugated to at least one colony-stimulating factor (CSF). Colony stimulating factors (CSFs) are growth factors that assist the bone marrow in making white blood cells. Some cancer treatments (e.g., chemotherapy) can affect white blood cells (which help fight infection); therefore, colony-stimulating factors may be introduced to help support white blood cell levels and strengthen the immune system. Colony-stimulating factors may also be used following a bone marrow transplant to help the new marrow start producing white blood cells. Representative examples of CSFs that may be used in the anti-CD98 ADCs of the invention include, but are not limited to erythropoietin (Epoetin), filgrastim (Neopogen (also known as granulocyte colony-stimulating factor (G-CSF); Amgen, Inc.), sargramostim (leukine (granulocyte-macrophage colony-stimulating factor and GM-CSF); Genzyme Corporation), promegapoietin, and Optelvekin (recombinant IL-11; Pfizer, Inc.). Thus, in one embodiment, the invention provides an ADC comprising an anti-CD98 antibody described herein and a CSF.

### 4. Gene Therapy

The anti-CD98 antibody of the invention may be conjugated to at least one nucleic acid (directly or indirectly via a carrier) for gene therapy. Gene therapy generally refers to the introduction of genetic material into a cell whereby the genetic material is designed to treat a disease. As it pertains to immunomodulatory agents, gene therapy is used to stimulate a subject's natural ability to inhibit cancer cell proliferation or kill cancer cells. In one embodiment, the anti-CD98 ADC of the invention comprises a nucleic acid encoding a functional, therapeutic gene that is used to replace a mutated or otherwise dysfunctional (e.g, truncated) gene associated with cancer. In other embodiments, the anti-CD98 ADC of the invention comprises a nucleic acid that encodes for or otherwise provides for the production of a therapeutic protein to treat cancer. The nucleic acid that encodes the therapeutic gene may be directly conjugated to the anti-CD98 antibody, or alternatively, may be conjugated to the anti-CD98 antibody through a carrier. Examples of carriers that may be used to deliver a nucleic acid for gene therapy include, but are not limited to, viral vectors or liposomes.

### 5. Alkylating Agents

The anti-CD98 antibodies of the invention may be conjugated to one or more alkylating agent(s). Alkylating agents are a class of antineoplastic compounds that attaches an alkyl group to DNA. Examples of alkylating agents that may be used in the ADCs of the invention include, but are not limited to, alkyl sulfonates, ethylenimimes, methylamine derivatives, epoxides, nitrogen mustards, nitrosoureas, triazines, and hydrazines.

a. Alkyl Sulfonates

The anti-CD98 antibodies of the invention may be conjugated to at least one alkyl sulfonate. Alkyl sulfonates are a subclass of alkylating agents with a general formula: R—SO₂—O—R¹, wherein R and R¹ are typically alkyl or aryl groups. A representative example of an alkyl sulfonate includes, but is not limited to, busulfan (Myleran, GlaxoSmithKline; Busulfex IV, PDL BioPharma, Inc.).

b. Nitrogen Mustards

The anti-CD98 antibodies of the invention may be conjugated to at least one nitrogen mustard. Representative examples of this subclass of anti-cancer compounds include, but are not limited to chlorambucil (Leukeran, GlaxoSmithKline), cyclophosphamide (Cytoxan, Bristol-Myers Squibb; Neosar, Pfizer. Inc.), estramustine (estramustine phosphate sodium or Estracyt), Pfizer, Inc.), ifosfamide (Ifex, Bristol-Myers Squibb), mechlorethamine (Mustargen, Lundbeck Inc.), and melphalan (Alkeran or L-Pam or phenylalanine mustard; GlaxoSmithKline).

c. Nitrosoureas

The anti-CD98 antibody of the invention may be conjugated to at least one nitrosourea. Nitrosoureas are a subclass of alkylating agents that are lipid soluble. Representative examples include, but are not limited to, carmustine (BCNU [also known as BiCNU, N,N-Bis(2-chloroethyl)-N-nitrosourea, or 1,3-bis (2-chloroethyl)-/-nitrosourea], Bristol-Myers Squibb), fotemustine (also known as Muphoran), lomustine (CCNU or 1-(2-chloro-ethyl)-3-cyclohexyl-1-nitrosourea, Bristol-Myers Squibb), nimustine (also known as ACNU), and streptozocin (Zanosar, Teva Pharmaceuticals).

d. Triazines and Hydrazines

The anti-CD98 antibody of the invention may be conjugated to at least one triazine or hydrazine. Triazines and hydrazines are a subclass of nitrogen-containing alkylating agents. In some embodiments, these compounds spontaneously decompose or can be metabolized to produce alkyl diazonium intermediates that facilitate the transfer of an alkyl group to nucleic acids, peptides, and/or polypeptides, thereby causing mutagenic, carcinogenic, or cytotoxic effects. Representative examples include, but are not limited to dacarbazine (DTIC-Dome. Bayer Healthcare Pharmaceuticals Inc.), procarbazine (Mutalane, Sigma-Tau Pharmaceuticals, Inc.), and temozolomide (Temodar, Sobering Plough).

e. Other Alkylating Agents

The anti-CD98 antibodies of the invention may be conjugated to at least one ethylenimine, methylamine derivative, or epoxide. Ethylenimines are a subclass of alkylating agents that typically containing at least one aziridine ring. Epoxides represent a subclass of alkylating agents that are characterized as cyclic ethers with only three ring atoms.

Representatives examples of ethylenimines include, but are not limited to thiopeta (Thioplex, Amgen), diaziquone (also known as aziridinyl benzoquinone (AZQ)), and mitomycin C. Mitomycin C is a natural product that contains an aziridine ring and appears to induce cytotoxicity through crosslinking DNA (Dorr R T, et al. Cancer Res. 1985; 45:3510; Kennedy K A, et al Cancer Res. 1985; 45:3541). Representative examples of methylamine derivatives and their analogs include, but are not limited to, altretamine (Hexalen, MGI Pharma, Inc.), which is also known as hexamethylamine and hexastat. Representative examples of epoxides of this class of anti-cancer compound include, but are not limited to dianhydrogalactitol. Dianliydrogalactitol (1,2:5,6-dianhydrodulcitol) is chemically related to the aziridines and generally facilitate the transfer of an alkyl group through a similar mechanism as described above. Dibromodulcitol is hydrolyzed to dianhydrogalactitol and thus is a pro-drug to an epoxide (Sellei C, et al. Cancer Chemother Rep. 1969; 53:377).

### 6. Antiangiogenic Agents

In one aspect, the anti-CD98 antibodies described herein are conjugated to at least one antiangiogenic agent. Antiangiogenic agents inhibit the growth of new blood vessels. Antiangiogenic agents exert their effects in a variety of way s. In some embodiments, these agents interfere with the ability of a growth factor to reach its target. For example, vascular endothelial growth factor (VEGF) is one of the primary proteins involved in initiating angiogenesis by binding to particular receptors on a cell surface. Thus, certain antiangiogenic agents, that prevent the interaction of VEGF with its cognate receptor, prevent VEGF from initiating angiogenesis. In other embodiments, these agents interfere with intracellular signaling cascades. For example, once a particular receptor on a cell surface has been triggered, a cascade of other chemical signals is initiated to promote the growth of blood vessels. Thus, certain enzymes, for example, some tyrosine kinases, that are known to facilitate intracellular signaling cascades that contribute to, for example, cell proliferation, are targets for cancer treatment. In other embodiments, these agents interfere with intercellular signaling cascades. Yet, in other embodiments, these agents disable specific targets that activate and promote cell growth or by directly interfering with the growth of blood vessel cells. Angiogenesis inhibitory properties have been discovered in more than 300 substances with numerous direct and indirect inhibitory effects.

Representative examples of antiangiogenic agents that may be used in the ADCs of the invention include, but are not limited to, angiostatin, ABX EGF, C1-1033, PKI-166, EGF vaccine. EKB-569, GW2016, ICR-62, EMD 55900, CP358, PD153035, AG1478, IMC-C225 (Erbitux, ZD1839 (Iressa), OSI-774, Erlotinib (tarceva), angiostatin, arrestin, endostatin, BAY 12-9566 and w/fluorouracil or doxorubicin, canstatin, carboxyamidotriozole and with paclitaxel, EMD 121974, S-24, vitaxin, dimethylxanthenone acetic acid, IM862, Interleukin-12, Interleukin-2, NM-3, HuMV833, PTK787, RhuMab, angiozvmc (ribozyme), IMC-1C11, Neovastat, marimstat, prinomastat, BMS-275291.COL-3, MM1270, SU101, SU6668, SU11248, SU5416, with paclitaxel, with gemcitabine and cisplatin, and with irinotecan and cisplatin and with radiation, tecogalan, temozolomide and PEG interferon α2b, tetrathiomolybdate, TNP-470, thalidomide, CC-5013 and with taxotere, tumstatin, 2-methoxyestradiol, VEGF trap, mTOR inhibitors (deforolimus, everolimus (Afinitor, Novartis Pharmaceutical Corporation), and temsirolimus (Torisel, Pfizer, Inc.)), kinase inhibitors (e.g., erlotinib (Tarceva, Genentech, Inc.), imatinib (Gleevec, Novartis Pharmaceutical Corporation), gefitinib (Iressa, AstraZeneca Pharmaceuticals), dasatinib (Sprycel, Bristol-Myers Squibb), sunitinib (Sutent, Pfizer, Inc.), nilotinib (Tasigna, Novartis Pharmaceutical Corporation), lapatinib (Tykerb, GlaxoSmithKline Pharmaceuticals), sorafenib (Nexavar, Bayer and Onyx), phosphoinositide 3-kinases (PI3K). Osimcrtinib, Cobimetinib, Trametinib, Dabrafenib, Dinaciclib).

### 7. Antimetabolites

The anti-CD98 antibodies of the invention may be conjugated to at least one antimetabolite. Antimetabolites are types of chemotherapy treatments that are very similar to normal substances within the cell. When the cells incorporate an antimetabolite into the cellular metabolism, the result is negative for the cell, e.g., the cell is unable to divide. Antimetabolites are classified according to the substances with which they interfere. Examples of antimetabolites that may be used in the ADCs of the invention include, but are not limited to, a folic acid antagonist (e.g., methotrexate), a pyrimidine antagonist (e.g., 5-Fluorouracil, Foxuridine, Cytarabine, Capecitabine, and Gemcitabine), a purine antagonist (e.g., 6-Mercaptopurine and 6-Thioguanine) and an adenosine deaminase inhibitor (e.g., Cladribine, Fludarabine, Nelarabine and Pentostatin), as described in more detail below.

a. Antifolates

The anti-CD98 antibodies of the invention may be conjugated to at least one antifolate. Antifolates are a subclass of antimetabolites that are structurally similar to folate. Representative examples include, but are not limited to, methotrexate, 4-amino-folic acid (also known as aminopterin and 4-aminopteroic acid), lometrexol (LMTX), pemetrexed (Alimpta, Eli Lilly and Company), and trimetrexate (Neutrexin, Ben Venue Laboratories, Inc.)

b. Purine Antagonists

The anti-CD98 antibodies of the invention may be conjugated to at least one purine antagonist. Purine analogs are a subclass of antimetabolites that are structurally similar to the group of compounds known as purines. Representative examples of purine antagonists include, but are not limited to, azathioprine (Azasan, Salix; Imuran, GlaxoSmithKline), cladribine (Leustatin [also known as 2-CdA], Janssen Biotech, Inc.), mercaptopurine (Purinethol [also known as 6-mercaptoethanol], GlaxoSmithKline), fludarabine (Fludara, Genzyme Corporation), pentostatin (Nipent, also known as 2′-deoxycoformycin (DCF)), 6-thioguanine (Lanvis [also known as thioguanine]. GlaxoSmithKline).

c. Pyrimidine Antagonists

The anti-CD98 antibodies of the invention may be conjugated to at least one pyrimidine antagonist. Pyrimidine antagonists are a subclass of antimetabolites that are structurally similar to the group of compounds known as purines. Representative examples of pyrimidine antagonists include, but are not limited to azacitidine (Vidaza, Celgene Corporation), capecitabine (Xeloda, Roche Laboratories), Cytarabine (also known as cytosine arabinoside and arabinosylcytosine. Bedford Laboratories), decitabine (Dacogen, Eisai Pharmaceuticals), 5-fluorouracil (Adrucil, Teva Pharmaceuticals; Efudex, Valeant Pharmaceuticals, Inc), 5-fluoro-2′-deoxyuridine 5′-phosphate (FdUMP), 5-fluorouridine triphosphate, and gemcitabine (Gemzar, Eli Lilly and Company).

### 8. Boron-Containing Agents

The anti-CD98 antibody of the invention may be conjugated to at least one boron containing agent. Boron-containing agents comprise a class of cancer therapeutic compounds which interfere with cell proliferation. Representative examples of boron containing agents include, but are not limited, to borophycin and bortezomib (Velcade, Millenium Pharmaceuticals).

### 9. Chemoprotective Agents

The anti-CD98 antibodies of the invention may be conjugated to at least one chemoprotective agent. Chemoprotective drugs are a class of compounds, which help protect the body against specific toxic effects of chemotherapy. Chemoprotective agents may be administered with various chemotherapies in order to protect healthy cells from the toxic effects of chemotherapy drugs, while simultaneously allowing the cancer cells to be treated with the administered chemotherapeutic. Representative chemoprotective agents include, but are not limited to amifostine (Ethyol, Medimmune, Inc.), which is used to reduce renal toxicity associated with cumulative doses of cisplatin, dexrazoxane (Totect, Apricus Pharma: Zinecard), for the treatment of extravasation caused by the administration of anthracycline (Totect), and for the treatment of cardiac-related complications caused by the administration of the antitumor antibiotic doxorubicin (Zinecard), and mesna (Mesnex, Bristol-Myers Squibb), which is used to prevent hemorrhagic cystitis during chemotherapy treatment with ifocfamide.

### 10. Hormone Agents

The anti-CD98 antibody of the invention may be conjugated to at least one hormone agent. A hormone agent (including synthetic hormones) is a compound that interferes with the production or activity of endogenously produced hormones of the endocrine system. In some embodiments, these compounds interfere with cell growth or produce a cytotoxic effect. Non-limiting examples include androgens, estrogens, medroxyprogesterone acetate (Provera, Pfizer, Inc.), and progestins.

### 11. Antihormone Agents

The anti-CD98 antibodies of the invention may be conjugated to at least one antihormone agent. An “antihormone” agent is an agent that suppresses the production of and/or prevents the function of certain endogenous hormones. In one embodiment, the antihormone agent interferes with the activity of a hormone selected from the group comprising androgens, estrogens, progesterone, and gonadotropin-releasing hormone, thereby interfering with the growth of various cancer cells. Representative examples of antihormone agents include, but are not limited to, aminoglutethimide, anastrozole (Arimidex, AstraZeneca Pharmaceuticals), bicalutamide (Casodex, AstraZeneca Pharmaceuticals), cyproterone acetate (Cyprostat, Bayer PLC), degarelix (Firmagon, Ferring Pharmaceuticals), exemestane (Aromasin, Pfizer Inc.), flutamide (Drogcnil, Schering-Plough Ltd), fulvestrant (Faslodex, AstraZeneca Pharmaceuticals), goserelin (Zolodex, AstraZeneca Pharmaceuticals), letrozole (Femara, Novartis Pharmaceuticals Corporation), leuprolide (Prostap), lupron, medroxyprogesterone acetate (Provera, Pfizer Inc.), Megestrol acetate (Megace, Bristol-Myers Squibb Company), tamoxifen (Nolvadex, AstraZeneca Pharmaceuticals), and triptorelin (Decapetyl, Ferring).

### 12. Corticosteroids

The anti-CD98 antibodies of the invention may be conjugated to at least one corticosteroid. Corticosteroids may be used in the ADCs of the invention to decrease inflammation. An example of a corticosteroid includes, but is not limited to, a glucocorticoid, for example, prednisone (Deltasone, Pharmacia & Upjohn Company, a division of Pfizer. Inc.).

### 13. Photoactive Therapeutic Agents

The anti-CD98 antibodies of the invention may be conjugated to at least one photoactive therapeutic agent. Photoactive therapeutic agents include compounds that can be deployed to kill treated cells upon exposure to electromagnetic radiation of a particular wavelength. Therapeutically relevant compounds absorb electromagnetic radiation at wavelengths which penetrate tissue. In preferred embodiments, the compound is administered in a non-toxic form that is capable of producing a photochemical effect that is toxic to cells or tissue upon sufficient activation. In other preferred embodiments, these compounds are retained by cancerous tissue and are readily cleared from normal tissues. Non-limiting examples include various chromagens and dyes.

### 14. Oligonucleotides

The anti-CD98 antibodies of the invention may be conjugated to at least one oligonucleotide. Oligonucleotides are made of short nucleic acid chains that work by interfering with the processing of genetic information. In some embodiments, the oligonucleotides for use in ADCs are unmodified single-stranded and/or double-stranded DNA or RNA molecules, while in other embodiments, these therapeutic oligonucleotides are chemically-modified single-stranded and/or double-stranded DNA or RNA molecules. In one embodiment, the oligonulceotides used in the ADCs are relatively short (19-25 nucleotides) and hybridize to a unique nucleic acid sequence in the total pool of nucleic acid targets present in cells. Some of the important oligonucleotide technologies include the antisense oligonucleotides (including RNA interference (RNAi)), aptamers, CpG oligonucleotides, and ribozymes.

a. Antisense Oligonucleotides

The anti-CD98 antibody of the invention may be conjugated to at least one antisense oligonucleotide. Antisense oligonucleotides are designed to bind to RNA through Watson-Crick hybridization. In some embodiments the antisense oligonucleotide is complementary to a nucleotide encoding a region, domain, portion, or segment of CD98. In some embodiments, the antisense oligonucleotide comprises from about 5 to about 100 nucleotides, from about 10 to about 50 nucleotides, from about 12 to about 35, and from about 18 to about 25 nucleotides. In some embodiments, the oligonucleotide is at least 50%, at least 60%, at least 70%, at least 80%, at least 90%, at least 95%, at least 96%, at least 97%, at least 98%, at least 99%, or at least 100% homologous to a region, portion, domain, or segment of the CD98 gene. In some embodiments there is substantial sequence homology over at least 15, 20, 25, 30, 35,40,50, or 100 consecutive nucleotides of the CD98 gene. In preferred embodiments, the size of these antisense oligonucleotides ranges from 12 to nucleotides in length, with the majority of antisense oligonucleotides being 18 to 21 nucleotides in length. There are multiple mechanisms that can be exploited to inhibit the function of the RNA once the oligonucleotide binds to the target RNA (Crooke S T. (1999). Biochim. Biophys. Acta, 1489, 30-42). The best-characterized antisense mechanism results in cleavage of the targeted RNA by endogenous cellular nucleases, such as RNase H or the nuclease associated with the RNA interference mechanism. However, oligonucleotides that inhibit expression of the target gene by non-catalytic mechanisms, such as modulation of splicing or translation arrest, can also be potent and selective modulators of gene function.

Another RNase-dependent antisense mechanism that has recently received much attention is RNAi (Fire et at. (1998). Nature. 391, 806-811; Zamore P D. (2002). Science, 296, 1265-1269.). RNA interference (RNAi) is a post-transcriptional process where a double stranded RNA inhibits gene expression in a sequence specific fashion. In some embodiments, the RNAi effect is achieved through the introduction of relatively longer double-stranded RNA (dsRNA), while in preferred embodiments, this RNAi effect is achieved by the introduction of shorter double-stranded RNAs, e.g. small interfering RNA (siRNA) and/or microRNA (miRNA). In yet another embodiment RNAi can also be achieved by introducing of plasmid that generates dsRNA complementary to target gene. In each of the foregoing embodiments, the double-stranded RNA is designed to interfere with the gene expression of a particular the target sequence within cells. Generally, the mechanism involves conversion of dsRNA into short RNAs that direct ribonucleases to homologous mRNA targets (summarized, Ruvkun, Science 2294:797 (2001)), which then degrades the corresponding endogenous mRNA, thereby resulting in the modulation of gene expression. Notably, dsRNA has been reported to have anti-proliferative properties, which makes it possible also to envisage therapeutic applications (Aubel et al, Proc. Natl. Acad Sci., USA 88:906 (1991)). For example, synthetic dsRNA has been shown to inhibit tumor growth in mice (Levy et al. Proc. Nat. Acad. Sci. USA, 62:357-361 (1969)), is active in the treatment of leukemic mice (Zeleznick et al., Proc. Soc. Exp. Biol. Med 130:126-128 (1969)), and inhibits chemically induced tumorigenesis in mouse skin (Gelboin et al, Science 167:205-207 (1970)). Thus, in a preferred embodiment, the invention provides for the use of antisense oligonucleotides in ADCs for the treatment of breast cancer. In other embodiments, the invention provides compositions and methods for initiating antisense oligonucleotide treatment, wherein dsRNA interferes with target cell expression of CD98 at the mRNA level, dsRNA, as used above, refers to naturally-occurring RNA, partially purified RNA, recombinantly produced RNA, synthetic RNA, as well as altered RNA that differs from naturally-occurring RNA by the inclusion of non-standard nucleotides, non-nucleotide material, nucleotide analogs (e.g. locked nucleic acid (LNA)), deoxyribonucleotides, and any combination thereof. RNA of the invention need only be sufficiently similar to natural RNA that it has the ability to mediate the antisense oligonucleotide-based modulation described herein.

b. Aptamers

The anti-CD98 antibodies of the invention may be conjugated to at least one aptamer. An aptamer is a nucleic acid molecule that has been selected from random pools based on its ability to bind other molecules. Like antibodies, aptamers can bind target molecules with extraordinary affinity and specificity. In many embodiments, aptamers assume complex, sequence-dependent, three-dimensional shapes that allow them to interact with a target protein, resulting in a tightly bound complex analogous to an antibody-antigen interaction, thereby interfering with the function of said protein. The particular capacity of aptamers to bind tightly and specifically to their target protein underlines their potential as targeted molecular therapies.

c. CpG oligonucleotides

The anti-CD98 antibodies of the invention may be conjugated to at least one CpG oligonucleotide. Bacterial and viral DNA are known to be a strong activators of both the innate and specific immunity in humans. These immunologic characteristics have been associated with unmethylated CpG dinucleotide motifs found in bacterial DNA. Owing to the fact that these motifs are rare in humans, the human immune system has evolved the ability to recognize these motifs as an early indication of infection and subsequently initiate immune responses. Therefore, oligonucleotides containing this CpG motif can be exploited to initiate an antitumor immune response.

d. Ribozymes

The anti-CD98 antibody of the invention may be conjugated to at least one ribozyme. Ribozymes are catalytic RNA molecules ranging from about 40 to 155 nucleotides in length. The ability of ribozymes to recognize and cut specific RNA molecules makes them potential candidates for therapeutics. A representative example includes angiozvmc.

### 15. Radionuclide Agents (Radioactive Isotopes)

The anti-CD98 antibodies of the invention may be conjugated to at least one radionuclide agent. Radionuclide agents comprise agents that are characterized by an unstable nucleus that is capable of undergoing radioactive decay. The basis for successful radionuclide treatment depends on sufficient concentration and prolonged retention of the radionuclide by the cancer cell. Other factors to consider include the radionuclide half-life, the energy of the emitted particles, and the maximum range that the emitted particle can travel. In preferred embodiments, the therapeutic agent is a radionuclide selected from the group consisting of ¹¹¹In, ¹⁷⁷Lu, ²¹²Bi, ²¹³Bi, ²¹¹At, ⁶²Cu, ⁶⁴Cu, ⁶⁷Cu, ⁹⁰Y, ¹²⁵I, ¹³¹I, ³²P, ³³P, ⁴⁷Sc, ¹¹¹Ag, ⁶⁷Ga, ¹⁴²Pr, ¹⁵³Sm, ¹⁶¹Tb, ¹⁶⁶Dy, ¹⁸⁶Ho, ¹⁸⁶Re, ¹⁸⁸Re, ¹⁸⁹Re, ²¹²Pb, ²²³Ra, ²²⁵Ac, ⁵⁹Fe, ⁷⁵Se, ⁷⁷As, ⁸⁹Sr, ⁹⁹Mo, ¹⁰⁵Rh, ¹⁰⁹Pd, ¹⁴³Pr, ¹⁴⁹Pm, ¹⁶⁹Er, ¹⁹⁴Ir, ¹⁹⁸Au, ¹⁹⁹Au, and ²¹¹Pb. Also preferred are radionuclides that substantially decay with Auger-emitting particles. For example. Co-58, Ga-67, Br-80m. Tc-99m, Rh-103m, Pt-109, In-111 1, Sb-119, I-125, Ho-161, Os-189m and Ir-192. Decay energies of useful beta-particle-emitting nuclides are preferably Dy-152, At-211, Bi-212, Ra-223, Rn-219, Po-215, Bi-211, Ac-225, Fr-221, At-217, Bi-213 and Fm-255. Decay energies of useful alpha-particle-emitting radionuclides are preferably 2.000-10,000 keV, more preferably 3,000-8,000 ke V, and most preferably 4,000-7,000 ke V. Additional potential radioisotopes of use include ¹¹C, ¹³N, ¹⁵0, ⁷⁵Br, ¹⁹⁸AU, ²²⁴AC, ¹²⁶I, ¹³³I, ⁷⁷Br, ^{113m}In, ⁹⁵Ru, ⁹⁷Ru, ¹⁰³Ru, ¹⁰⁵Ru, ¹⁰⁷Hg, ²⁰³Hg, ^{121m}Te, ^{122m}Te, ^{125m}Te·¹⁶⁵Tm, ¹⁶⁷Tm, ¹⁶⁸Tm, ¹⁹⁷Pt, ¹⁰⁹Pd, ¹⁰⁵Rh, ¹⁴²Pr, ¹⁶¹Pr, ¹⁶¹Tb, ¹⁶⁶Ho, ¹⁹⁹Au, ⁵⁷Co, ⁵⁸Co, ⁵¹Cr, ⁵⁹Fe, ⁷⁵Se, ²⁰¹Tl, ²²⁵Ac, ⁷⁶Br, ¹⁶⁹Yb, and the like.

### 16. Radiosensitizers

The anti-CD98 antibodies of the invention may be conjugated to at least one radiosensitizer. The term “radiosensitizer.” as used herein, is defined as a molecule, preferably a low molecular weight molecule, administered to animals in therapeutically effective amounts to increase the sensitivity of the cells to be radiosensitized to electromagnetic radiation and/or to promote the treatment of diseases that are treatable with electromagnetic radiation. Radiosensitizers are agents that make cancer cells more sensitive to radiation therapy, while typically having much less of an effect on normal cells. Thus, the radiosensitizer can be used in combination with a radiolabeled antibody or ADC. The addition of the radiosensitizer can result in enhanced efficacy when compared to treatment with the radiolabeled antibody or antibody fragment alone. Radiosensitizers are described in D. M. Goldberg (ed.). Cancer Therapy with Radiolabeled Antibodies. CRC Press (1995). Examples of radiosensitizers include gemcitabine, 5-fluorouracil, taxane, and cisplatin.

Radiosensitizers may be activated by the electromagnetic radiation of X-rays. Representative examples of X-ray activated radiosensitizers include, but are not limited to, the following: metronidazole, misonidazole, desmethylmisonidazole, pimonidazole, etanidazole, nimorazole, mitomycin C, RSU 1069, SR 4233, E09, RB 6145, nicotinamide, 5-bromodeoxyuridine (BUdR), 5-iododeoxyuridine (IUdR), bromodeoxycytidine, fluorodeoxyuridine (FUdR), hydroxyurea, cisplatin, and therapeutically effective analogs and derivatives of the same. Alternatively, radiosensitizers may be activated using photodynamic therapy (PDT). Representative examples of photodynamic radiosensitizers include, but are not limited to, hematoporphyrin derivatives, Photofrin(r), benzoporphyrin derivatives, NPe6, tin etioporphyrin (SnET2), pheoborbide a, bacteriochlorophyll a, naphthalocyanines, phthalocyanines, zinc phthalocyanine, and therapeutically effective analogs and derivatives of the same.

### 16. Topoisomerase Inhibitors

The anti-CD98 antibodies of the invention may be conjugated to at least one topoisomerase inhibitor. Topoisomerase inhibitors are chemotherapy agents designed to interfere with the action of topoisomerase enzymes (topoisomerase I and II), which are enzymes that control the changes in DNA structure by catalyzing then breaking and rejoining of the phosphodiester backbone of DNA strands during the normal cell cycle. Representative examples of DNA topoisomerase I inhibitors include, but are not limited to, camptothecins and its derivatives irinotecan (CPT-11, Camptosar, Pfizer. Inc.) and topotecan (Hycamtin, GlaxoSmithKline Pharmaceuticals). Representative examples of DNA topoisomerase II inhibitors include, but are not limited to, amsacrine, daunorubicin, doxotrubicin, epipodophyllotoxins, ellipticines, epirubicin, etoposide, razoxane, and teniposide.

### 17. Kinase Inhibitors

The anti-CD98 antibodies of the invention may be conjugated to at least one kinase inhibitor. By blocking the ability of protein kinases to function, tumor growth may be inhibited. Examples of kinase inhibitors that may be used in the ADCs of the invention include, but are not limited to, Axitinib, Bosutinib, Cediranib, Dasatinib, Erlotinib, Gefitinib, Imatinib, Lapatinib, Lestaurtinib, Nilotinib, Semaxanib, Sunitinib, Osimertinib, Cobimetinib, Trametinib, Dabrafenib, Dinaciclib, and Vandetanib.

### 18. Other Agents

Examples of other agents that may be used in the ADCs of the invention include, but are not limited to, abrin (e.g., abrin A chain), alpha toxin. Aleurites fordii proteins, amatoxin, crotin, curcin, dianthin proteins, diptheria toxin (e.g, diphtheria A chain and nonbinding active fragments of diphtheria toxin), deoxyribonuclease (Dnase), gelonin, mitogellin, modeccin A chain, Momordica charantia inhibitor, neomycin, onconase, phenomycin, Phytolaca americana proteins (PAPI, PAPII, and PAP-S), pokeweed antiviral protein, Pseudomonas endotoxin, Pseudomonas exotoxin (e.g. exotoxin A chain (from Pseudomonas aeruginosa)), restrictocin, ricin A chain, ribonuclease (Rnase), Sapaonaria officinalis inhibitor, saporin, alpha-sarcin, Staphylcoccal enterotoxin-A, tetanus toxin, cisplatin, carboplatin, and oxaliplatin (Eloxatin, Sanofi Aventis), proteasome inhibitors (e.g. PS-341 [bortezomib or Velcade]), HD AC inhibitors (vorinostat (Zolinza, Merck & Company, Inc.)), belinostat, entinostat, mocetinostat, and panobinostat). COX-2 inhibitors, substituted ureas, heat shock protein inhibitors (e g. Geldanamycin and its numerous analogs), adrenocortical suppressants, and the tricothecenes. (See, for example, WO 93/21232). Other agents also include asparaginase (Espar, Lundbcck Inc.), hydroxy-urea, levamisole, mitotane (Lysodren, Bristol-Myers Squibb), and tretinoin (Renova, Valeant Pharmaceuticals Inc.).

### III.C. Anti-CD98 ADCs: Other Exemplary Linkers

In addition to the linkers mentioned above, other exemplary linkers include, but are not limited to, 6-maleimidocaproyl, maleimidopropanoyl (“MP”), valine-citrulline (“val-cit” or “vc”), alanine-phenylalanine (“ala-phe”), p-aminobenzyloxycarbonyl (a “PAB”), N-Succinimidyl 4-(2-pyridylthio) pentanoate C(“SPP”), and 4-(N-maleimidomethyl)cyclohexane-1 carboxylate (“MCC”).

In one aspect, an anti-CD98 antibody is conjugated to a drug, (such as auristatin, e.g., MMAE), via a linker comprising maleimidocaproyl (“mc”), valine citrulline (val-cit or “vc”), and PABA (referred to as a “mc-vc-PABA linker”). Maleimidocaproyl acts as a linker to the anti-CD98 antibody and is not cleavable. Val-cit is a dipeptide that is an amino acid unit of the linker and allows for cleavage of the linker by a protease, specifically the protease cathepsin B. Thus, the val-cit component of the linker provides a means for releasing the auristatin from the ADC upon exposure to the intracellular environment. Within the linker, p-aminobenzylalcohol (PABA) acts as a spacer and is self immolative, allowing for the release of the MMAE. The structure of the mc-vc-PABA-MMAE linker is provided in FIG. 3.

As described above, suitable linkers include, for example, cleavable and non-cleavable linkers. A linker may be a “cleavable linker.” facilitating release of a drug. Nonlimiting exemplary cleavable linkers include acid-labile linkers (e.g., comprising hydrazone), protease-sensitive (e.g., peptidase-sensitive) linkers, photolabile linkers, or disulfide-containing linkers (Chari et al., Cancer Research 52:127-131 (1992): U.S. Pat. No. 5,208,020). A cleavable linker is typically susceptible to cleavage under intracellular conditions. Suitable cleavable linkers include, for example, a peptide linker cleavable by an intracellular protease, such as lysosomal protease or an endosomal protease. In exemplary embodiments, the linker can be a dipeptide linker, such as a valine-citrulline (val-cit) or a phenylalanine-lysine (phe-lys) linker.

Linkers are preferably stable extracellularly in a sufficient manner to be therapeutically effective. Before transport or delivery into a cell, the ADC is preferably stable and remains intact, i.e. the antibody remains conjugated to the drug moiety. Linkers that are stable outside the target cell may be cleaved at some efficacious rate once inside the cell. Thus, an effective linker will: (i) maintain the specific binding properties of the antibody; (ii) allow delivery, e.g., intracellular delivery, of the drug moiety; and (iii) maintain the therapeutic effect, e.g., cytotoxic effect, of a drug moiety.

In one embodiment, the linker is cleavable under intracellular conditions, such that cleavage of the linker sufficiently releases the drug from the antibody in the intracellular environment to be therapeutically effective. In some embodiments, the cleavable linker is pH-sensitive, i.e., sensitive to hydrolysis at certain pH values. Typically, the pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond (see, e.g., U.S. Pat. No. 5,622,929).

In other embodiments, the linker is cleavable under reducing conditions (e.g., a disulfide linker). A variety of disulfide linkers are known in the art, including, for example, those that can be formed using SATA (N-succinimidyl-5-acetylthioacetate), SPDP (N-succinimidyl-3-(2-pyridyldithio)propionate), SPDB (N-succinimidyl-3-(2-pyridyldithio)butyrate) and SMPT (N-succinimidyloxycarbonyl-alpha-methyl-alpha-(2-pyridyl-dithio)toluene), SPDB and SMPT. (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.).

In some embodiments, the linker is cleavable by a cleaving agent, e.g., an enzyme, that is present in the intracellular environment (e.g., within a lysosome or endosome or caveolea). The linker can be, e.g., a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin, all of which are known to hydrolyze dipeptide drug derivatives resulting in the release of active drug inside target cells (see, e.g., Dubowchik and Walker, 1999. Pharm. Therapeutics 83:67-123). Most typical are peptidyl linkers that are cleavable by enzymes that are present in CD98-expressing cells. Examples of such linkers are described, e.g., in U.S. Pat. No. 6,214,345, incorporated herein by reference in its entirety and for all purposes. In a specific embodiment, the peptidyl linker cleavable by an intracellular protease is a Val-Cit linker or a Phe-Lys linker (see, e.g., U.S. Pat. No. 6,214,345, which describes the synthesis of doxorubicin with the val-cit linker). One advantage of using intracellular proteolytic release of the therapeutic agent is that the agent is typically attenuated when conjugated and the serum stabilities of the conjugates are typically high.

In other embodiments, the linker is a malonate linker (Johnson et al., 1995. Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10): 1305-12).

In yet other embodiments, the linker unit is not cleavable and the drug is released, for example, by antibody degradation. See U.S. Publication No. 20050238649 incorporated by reference herein in its entirety. An ADC comprising a non-cleavable linker may be designed such that the ADC remains substantially outside the cell and interacts with certain receptors on a target cell surface such that the binding of the ADC initiates (or prevents) a particular cellular signaling pathway.

In some embodiments, the linker is substantially hydrophilic linker (e.g., PEG4Mal and sulfo-SPDB). A hydrophilic linker may be used to reduce the extent to which the drug may be pumped out of resistant cancer cells through MDR (multiple drug resistance) or functionally similar transporters.

In other embodiments, upon cleavage, the linker functions to directly or indirectly inhibit cell growth and/or cell proliferation. For example, in some embodiments, the linker, upon cleavage, can function as an intercalating agent, thereby inhibiting macromolecular biosynthesis (e.g. DNA replication, RNA transcription, and/or protein synthesis).

In other embodiments, the linker is designed to facilitate bystander killing (the killing of neighboring cells) through diffusion of the linker-drug and/or the drug alone to neighboring cells. In other, embodiments, the linker promotes cellular internalization.

The presence of a sterically hindered disulfide can increase the stability of a particular disulfide bond, enhancing the potency of the ADC. Thus, in one embodiment, the linker includes a sterically hindered disulfide linkage. A sterically hindered disulfide refers to a disulfide bond present within a particular molecular environment, wherein the environment is characterized by a particular spatial arrangement or orientation of atoms, typically within the same molecule or compound, which prevents or at least partially inhibits the reduction of the disulfide bond. Thus, the presence of bulky (or sterically hindering) chemical moieties and/or bulky amino acid side chains proximal to the disulfide bond prevents or at least partially inhibits the disulfide bond from potential interactions that would result in the reduction of the disulfide bond.

Notably, the aforementioned linker types are not mutually exclusive. For example, in one embodiment, the linker used in the anti-CD98 ADCs described herein is a non-cleavable linker that promotes cellular internalization.

In some embodiments, a linker component comprises a “stretcher unit” that links an antibody to another linker component or to a drug moiety. An illustrative stretcher unit described in U.S. Pat. No. 8,309,093, incorporated by reference herein. In certain embodiments, the stretcher unit is linked to the anti-CD98 antibody via a disulfide bond between a sulfur atom of the anti-CD98 antibody unit and a sulfur atom of the stretcher unit. A representative stretcher unit of this embodiment is depicted in U.S. Pat. No. 8,309,093, incorporated by reference herein. In yet other embodiments, the stretcher contains a reactive site that can form a bond with a primary or secondary amino group of an antibody. Examples of these reactive sites include but are not limited to, activated esters such as succinimide esters, 4 nitrophenyl esters, pentafluorophenyl esters, tetrafluorophenyl esters, anhydrides, acid chlorides, sulfonyl chlorides, isocyanates and isothiocyanates. Representative stretcher units of this embodiment are depicted in U.S. Pat. No. 8,309,093, incorporated by reference herein.

In some embodiments, the stretcher contains a reactive site that is reactive to a modified carbohydrate's (—CHO) group that can be present on an antibody. For example, a carbohydrate can be mildly oxidized using a reagent such as sodium periodate and the resulting (—CHO) unit of the oxidized carbohydrate can be condensed with a Stretcher that contains a functionality such as a hydrazide, an oxime, a primary or secondary amine, a hydrazine, a thiosemicarbazone, a hydrazine carboxylate, and an arylhydrazide such as those described by Kaneko et al., 1991, Bioconjugate Chem. 2:133-41. Representative Stretcher units of this embodiment are depicted in U.S. Pat. No. 8,309,093, incorporated by reference herein.

In some embodiments, a linker component comprises an “amino acid unit”. In some such embodiments, the amino acid unit allows for cleavage of the linker by a protease, thereby facilitating release of the drug from the immunoconjugate upon exposure to intracellular proteases, such as lysosomal enzymes (Doronina et al. (2003) Nat. Biotechnol. 21:778-784). Exemplary amino acid units include, but are not limited to, dipeptides, tripeptides, tetrapeptides, and pentapeptides. Exemplary dipeptides include, but are not limited to, valine-citrulline (vc or val-cit), alanine-phenylalanine (af or ala-phe); phenylalanine-lysine (fk or phe-lys); phenylalanine-homolysine (phe-homolys); and N-methyl-valine-citrulline (Me-val-cit). Exemplary tripeptides include, but are not limited to, glycine-valine-citrulline (gly-val-cit) and glycine-glycine-glycine (gly-gly-gly). An amino acid unit may comprise amino acid residues that occur naturally and/or minor amino acids and/or non-naturally occurring amino acid analogs, such as citrulline Amino acid units can be designed and optimized for enzymatic cleavage by a particular enzyme, for example, a tumor-associated protease, cathepsin B, C and D, or a plasmin protease.

In one embodiment, the amino acid unit is valine-citrulline (vc or val-cit). In another aspect, the amino acid unit is phenylalanine-lysine (i.e., fk). In yet another aspect of the amino acid unit, the amino acid unit is N-methylvaline-citrulline. In yet another aspect, the amino acid unit is 5-aminovaleric acid, homo phenylalanine lysine, tetraisoquinolinecarboxylate lysine, cyclohexylalanine lysine, isonipecotic acid lysine, beta-alanine lysine, glycine serine valine glutamine and isonipecotic acid.

Alternatively, in some embodiments, the amino acid unit is replaced by a glucuronide unit that links a stretcher unit to a spacer unit if the stretcher and spacer units are present, links a stretcher unit to the drug moiety if the spacer unit is absent, and links the linker unit to the drug if the stretcher and spacer units are absent. The glucuronide unit includes a site that can be cleaved by a β-glucuronidase enzyme (See also US 2012/0107332, incorporated by reference herein). In some embodiments, the glucuronide unit comprises a sugar moiety (Su) linked via a glycoside bond (—O′—) to a self-immolative group (Z) of the formula as depicted below (See also US 2012/0107332, incorporated by reference herein).

Su-O′—Z

The glycosidic bond (—O′—) is typically a β-glucuronidase-cleavage site, such as a bond cleavable by human, lysosomal β-glucuronidase. In the context of a glucuronide unit, the term “self-immolative group” refers to a di- or tri-functional chemical moiety that is capable of covalently linking together two or three spaced chemical moieties (i.e., the sugar moiety (via a glycosidic bond), a drug moiety (directly or indirectly via a spacer unit), and, in some embodiments, a linker (directly or indirectly via a stretcher unit) into a stable molecule. The self-immolative group will spontaneously separate from the first chemical moiety (e.g., the spacer or drug unit) if its bond to the sugar moiety is cleaved.

In some embodiments, the sugar moiety (Su) is cyclic hexose, such as a pyranose, or a cyclic pentose, such as a furanose. In some embodiments, the pyranose is a glucuronide or hexose. The sugar moiety is usually in the β-D conformation. In a specific embodiment, the pyranose is a β-D-glucuronide moiety (i.e., β-D-glucuronic acid linked to the self-immolative group —Z— via a glycosidic bond that is cleavable by β-glucuronidase). In some embodiments, the sugar moiety is unsubstituted (e.g., a naturally occurring cyclic hexose or cyclic pentose). In other embodiments, the sugar moiety can be a substituted β-D-glucuronide (i.e., glucuronic acid substituted with one or more group, such hydrogen, hydroxyl, halogen, sulfur, nitrogen or lower alkyl. In some embodiments, the glucuronide unit has one of the formulas as described in US 2012/0107332, incorporated by reference herein.

In some embodiments, the linker comprises a spacer unit (—Y—), which, when present, links an amino acid unit (or Glucuronide unit, see also US 2012/0107332, incorporated by reference herein) to the drug moiety when an amino acid unit is present. Alternately, the spacer unit links the stretcher unit to the drug moiety when the amino acid unit is absent. The spacer unit may also links the drug unit to the antibody unit when both the amino acid unit and stretcher unit are absent.

Spacer units are of two general types: non self-immolative or self-immolative. A non self-immolative spacer unit is one in which part or all of the spacer unit remains bound to the drug moiety after cleavage, particularly enzymatic, of an amino acid unit (or glucuronide unit) from the antibody-drug conjugate. Examples of a non self-immolative spacer unit include, but are not limited to a (glycine-glycine) spacer unit and a glycine spacer unit (see U.S. Pat. No. 8,309,093, incorporated by reference herein)). Other examples of self-immolative spacers include, but are not limited to, aromatic compounds that are electronically similar to the PAB group such as 2-aminoimidazol-5-methanol derivatives (Hay et al., 1999. Bioorg. Med. Chem. Lett. 9:2237) and ortho or para-aminobenzylacetals. Spacers can be used that undergo cyclization upon amide bond hydrolysis, such as substituted and unsubstituted 4-aminobutyric acid amides (Rodrigues et al., 1995. Chemistry Biology 2:223), appropriately substituted bicyclo[2.2.11 ] and bicyclo[2.2.2] ring systems (Storm et al., 1972, J. Amer. Chem. Soc. 94:5815) and 2-aminophenylpropionic acid amides (Amsberry et al., 1990. J. Org. Chem. 55:5867). Elimination of amine-containing drugs that are substituted at the α-position of glycine (Kingsbury et al., 1984, J. Med. Chem. 27:1447) are also examples of self-immolative spacers.

Other examples of self-immolative spacers include, but are not limited to, aromatic compounds that are electronically similar to the PAB group such as 2-aminoimidazol-5-methanol derivatives (see, e.g., Hay et al., 1999. Bioorg. Med Chem. Lett. 9:2237) and ortho or para-aminobenzylacetals. Spacers can be used that undergo cyclization upon amide bond hydrolysis, such as substituted and unsubstituted 4-aminobutyric acid amides (see, e.g., Rodrigues et al., 1995. Chemistry Biology 2:223), appropriately substituted bicyclo[2.2.1] and bicyclo[2.2.2] ring systems (see, e.g., Storm et al., 1972, J. Amer. Chem. Soc. 94:5815) and 2-aminophenylpropionic acid amides (see, e.g., Amsberry et al., 1990. J. Org. Chem. 55:5867). Elimination of amine-containing drugs that are substituted at the a-position of glycine (see, e.g., Kingsbury et al., 1984, J. Med. Chem. 27:1447) are also examples of self-immolative spacers.

Other suitable spacer units are disclosed in Published U.S. Patent Application No. 2005-0238649, the disclosure of which is incorporated by reference herein.

Another approach for the generation of ADCs involves the use of heterobifunctional cross-linkers which link the anti-CD98 antibody to the drug moiety. Examples of cross-linkers that may be used include N-succinimidyl 4-(5-nitro-2-pyridyldithio)-pentanoate or the highly water-soluble analog N-sulfosuccinimidyl 4-(5-nitro-2-pyridyldithio)-pentanoate, N-succinimidyl-4-(2-pyridyldithio) butyrate (SPDB), N-succinimidyl-4-(5-nitro-2-pyridyldithio) butyrate (SNPB), and N-sulfosuccinimidyl-4-(5-nitro-2-pyridyldithio) butyrate (SSNPB). N-succinimidyl-4-methyl-4-(5-nitro-2-pyridyldithio)pentanoate (SMNP), N-succinimidyl-4-(5-N,N-dimethylcarboxamido-2-pyridyldithio) butyrate (SCPB) or N-sulfosuccinimidyl4-(5-N,N-dimethylcarboxamido-2-pyridyldithio) butyrate (SSCPB)). The antibodies of the invention may be modified with the cross-linkers N-succinimidyl 4-(5-nitro-2-pyridyldithio)-pentanoate. N-sulfosuccinimidyl 4-(5-nitro-2-pyridyldithio)-pentanoate, SPDB, SNPB, SSNPB, SMNP, SCPB, or SSCPB can then react with a small excess of a particular drug that contains a thiol moiety to give excellent yields of an ADC. Preferably, the cross-linkers are compounds of the formula as depicted in U.S. Pat. No. 6,913,748, incorporated by reference herein.

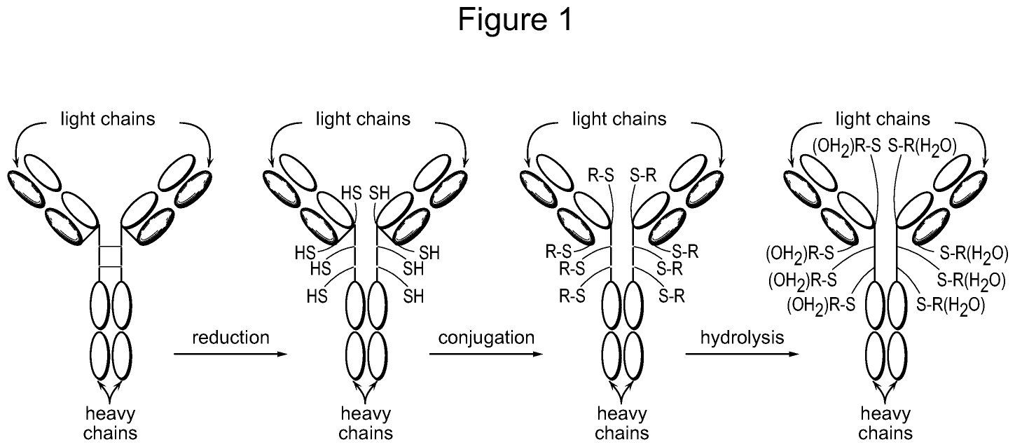

In one embodiment, charged linkers (also referred to as pro-charged linkers) are used to conjugate anti-CD98 antibodies to drugs to form ADCs. Charged linkers include linkers that become charged after cell processing. The presence of a charged group(s) in the linker of a particular ADC or on the drug after cellular processing provides several advantages, such as (i) greater water solubility of the ADC, (ii) ability to operate at a higher concentration in aqueous solutions, (iii) ability to link a greater number of drug molecules per antibody, potentially resulting in higher potency, (iv) potential for the charged conjugate species to be retained inside the target cell, resulting in higher potency, and (v) improved sensitivity of multidrug resistant cells, which would be unable to export the charged drug species from the cell. Examples of some suitable charged or pro-charged cross-linkers and their synthesis are shown in FIGS. 1 to 10 of U.S. Pat. No. 8,236,319, and are incorporated by reference herein. Preferably, the charged or pro-charged cross-linkers are those containing sulfonate, phosphate, carboxyl or quaternary amine substituents that significantly increase the solubility of the ADCs, especially for ADCs with 2 to 20 conjugated drugs. Conjugates prepared from linkers containing a pro-charged moiety would produce one or more charged moieties after the conjugate is metabolized in a cell.

Additional examples of linkers that can be used with the compositions and methods include valine-citrulline; maleimidocaproyl; amino benzoic acids; p-aminobenzylcarbamoyl (PAB); lysosomal enzyme-cleavable linkers: maleimidocaproyl-polyethylene glycol (MC(PEG)6-OH); N-methyl-valine citrulline; N-succinimidyl 4-(N-maleimidomethyl)cyclohexane-1-carboxylate (SMCC); N-Succinimidyl 4-(2-pyridyldithio)butanoate (SPDB); and N-Succinimidyl 4-(2-pyridylthio)pentanoate (SPP) (See also US 2011/0076232). Another linker for use in the invention includes an avidin-biotin linkage to provide an avidin-biotin-containing ADC (See also U.S. Pat. No. 4,676,980, PCT publication Nos. WO1992/022332A2, WO1994/016729A1, WO1995/015770A1, WO1997/031655A2, WO1998/035704A1, WO1999/019500A1, WO2001/09785A2. WO2001/090198A1 WO2003/093793A2, WO2004/050016A2, WO2005/081898A2, WO2006/083562A2, WO2006/089668A1. WO2007/150020A1, WO2008/135237A1, WO2010/11198A1, WO2011/057216A1, WO2011/058321A1, WO2012/027494A1, and EP77671B1), wherein some such linkers are resistant to biotinidase cleavage. Additional linkers that may be used in the invention include a cohesin/dockerin pair to provide a cohesion-dockerin-containing ADC (See PCT publication Nos. WO2008/097866A2, WO2008/097870A2, WO2008/103947A2, and WO2008/103953A2).

Additional linkers for use in the invention may contain non-peptide polymers (examples include, but are not limited to, polyethylene glycol, polypropylene glycol, polyoxyethylated polyols, polyvinyl alcohol, polysaccharides, dextran, polyvinyl ethyl ether. PLA (poly(lactic acid)). PLGA (poly(lactic acid-glycolic acid)), and combinations thereof, wherein a preferred polymer is polyethylene glycol) (See also PCT publication No. WO2011/000370). Additional linkers are also described in WO 20(4-010957, U.S. Publication No. 20060074008, U.S. Publication No. 20050238649, and U.S. Publication No. 20060024317, each of which is incorporated by reference herein in its entirety).

For an ADC comprising a maytansinoid, many positions on maytansinoids can serve as the position to chemically link the linking moiety. In one embodiment, maytansinoids comprise a linking moiety that contains a reactive chemical group are C-3 esters of maytansinol and its analogs where the linking moiety contains a disulfide bond and the chemical reactive group comprises a N-succinimidyl or N-sulfosuccinimidyl ester. For example, the C-3 position having a hydroxyl group, the C-14 position modified with hydroxymethyl, the C-15 position modified with hydroxy and the C-20 position having a hydroxy group are all useful. The linking moiety most preferably is linked to the C-3 position of maytansinol.

The conjugation of the drug to the antibody via a linker can be accomplished by any technique known in the art. A number of different reactions are available for covalent attachment of drugs and linkers to antibodies. This may be accomplished by reaction of the amino acid residues of the antibody, including the amine groups of lysine, the free carboxylic acid groups of glutamic and aspartic acid, the sulfhydryl groups of cysteine and the various moieties of the aromatic amino acids. One of the most commonly used non-specific methods of covalent attachment is the carbodiimide reaction to link a carboxy (or amino) group of a compound to amino (or carboxy) groups of the antibody. Additionally, bifunctional agents such as dialdehydes or imidoesters have been used to link the amino group of a compound to amino groups of an antibody. Also available for attachment of drugs to antibodies is the Schiff base reaction. This method involves the periodate oxidation of a drug that contains glycol or hydroxy groups, thus forming an aldehyde which is then reacted with the binding agent. Attachment occurs via formation of a Schiff base with amino groups of the antibody. Isothiocyanates can also be used as coupling agents for covalently attaching drugs to antibodies. Other techniques are known to the skilled artisan and within the scope of the invention.

In certain embodiments, an intermediate, which is the precursor of the linker, is reacted with the drug under appropriate conditions. In certain embodiments, reactive groups are used on the drug or the intermediate. The product of the reaction between the drug and the intermediate, or the derivatized drug, is subsequently reacted with the anti-CD98 antibody under appropriate conditions. The synthesis and structure of exemplary linkers, stretcher units, amino acid units, self-immolative spacer units are described in U.S. Patent Application Publication Nos. 20030083263, 20050238649 and 20060009751, each if which is incorporated herein by reference.

Stability of the ADC may be measured by standard analytical techniques such as mass spectroscopy, HPLC, and the separation/analysis technique LC/MS.

### IV. Purification of Anti-CD98 ADCs

Purification of the ADCs may be achieved in such a way that ADCs having certain DARs are collected. For example, HIC resin may be used to separate high drug loaded ADCs from ADCs having optimal drug to antibody ratios (DARs), e.g., a DAR of 4 or less. In one embodiment, a hydrophobic resin is added to an ADC mixture such that undesired ADCs, i.e., higher drug loaded ADCs, bind the resin and can be selectively removed from the mixture. In certain embodiments, separation of the ADCs may be achieved by contacting an ADC mixture (e.g., a mixture comprising a drug loaded species of ADC of 4 or less and a drug loaded species of ADC of 6 or more) with a hydrophobic resin, wherein the amount of resin is sufficient to allow binding of the drug loaded species which is being removed from the ADC mixture. The resin and ADC mixture are mixed together, such that the ADC species being removed (e.g., a drug loaded species of 6 or more) binds to the resin and can be separated from the other ADC species in the ADC mixture. The amount of resin used in the method is based on a weight ratio between the species to be removed and the resin, where the amount of resin used does not allow for significant binding of the drug loaded species that is desired. Thus, methods may be used to reduce the average DAR to less than 4. Further, the purification methods described herein may be used to isolate ADCs having any desired range of drug loaded species, e.g., a drug loaded species of 4 or less, a drug loaded species of 3 or less, a drug loaded species of 2 or less, a drug loaded species of 1 or less.

Certain species of molecule(s) binds to a surface based on hydrophobic interactions between the species and a hydrophobic resin. In one embodiment, method of the invention refers to a purification process that relies upon the intermixing of a hydrophobic resin and a mixture of ADCs, wherein the amount of resin added to the mixture determines which species (e.g., ADCs with a DAR of 6 or more) will bind. Following production and purification of an antibody from an expression system (e.g., a mammalian expression system), the antibody is reduced and coupled to a drug through a conjugation reaction. The resulting ADC mixture often contains ADCs having a range of DARs, e.g., 1 to 8. In one embodiment, the ADC mixture comprises a drug loaded species of 4 or less and a drug loaded species of 6 or more. According to the methods of the invention, the ADC mixture may be purified using a process, such as, but not limited to, a batch process, such that ADCs having a drug loaded species of 4 or less are selected and separated from ADCs having a higher drug load (e.g., ADCs having a drug loaded species of 6 or more). Notably, the purification methods described herein may be used to isolate ADCs having any desired range of DAR, e.g., a DAR of 4 or less, a DAR of 3 or less, and a DAR of 2 or less.

Thus, in one embodiment, an ADC mixture comprising a drug loaded species of 4 or less and a drug loaded species of 6 or more may be contacted with a hydrophobic resin to form a resin mixture, wherein the amount of hydrophobic resin contacted with the ADC mixture is sufficient to allow binding of the drug loaded species of 6 or more to the resin but does not allow significant binding of the drug load species of 4 or less; and removing the hydrophobic resin from the ADC mixture, such that the composition comprising ADCs is obtained, wherein the composition comprises less than 15% of the drug loaded species of 6 or more, and wherein the ADC comprises an antibody conjugated to a Bcl-xL inhibitor. In a separate embodiment, the method of the invention comprises contacting an ADC mixture comprising a drug loaded species of 4 or less and a drug loaded species of 6 or more with a hydrophobic resin to form a resin mixture, wherein the amount of hydrophobic resin contacted with the ADC mixture is sufficient to allow binding of the drug loaded species of 6 or more to the resin but does not allow significant binding of the drug load species of 4 or less: and removing the hydrophobic resin from the ADC mixture, such that the composition comprising ADCs is obtained, wherein the composition comprises less than 15% of the drug loaded species of 6 or more, and wherein the ADC comprises an antibody conjugated to a Bcl-xL inhibitor, wherein the hydrophobic resin weight is 3 to 12 times the weight of the drug loaded species of 6 or more in the ADC mixture.

The ADC separation method described herein method may be performed using a batch purification method. The batch purification process generally includes adding the ADC mixture to the hydrophobic resin in a vessel, mixing, and subsequently separating the resin from the supernatant. For example, in the context of batch purification, a hydrophobic resin may be prepared in or equilibrated to the desired equilibration buffer. A slurry of the hydrophobic resin may thus be obtained. The ADC mixture may then be contacted with the slurry to adsorb the specific species of ADC(s) to be separated by the hydrophobic resin. The solution comprising the desired ADCs that do not bind to the hydrophobic resin material may then be separated from the slurry, e.g., by filtration or by allowing the slurry to settle and removing the supernatant. The resulting slurry can be subjected to one or more washing steps. In order to elute bound ADCs, the salt concentration can be decreased. In one embodiment, the process used in the invention includes no more than 50 g of hydrophobic resin.

Thus, a batch method may be used to contact an ADC mixture comprising a drug loaded species of 4 or less and a drug loaded species of 6 or more with a hydrophobic resin to form a resin mixture, wherein the amount of hydrophobic resin contacted with the ADC mixture is sufficient to allow binding of the drug loaded species of 6 or more to the resin but does not allow significant binding of the drug load species of 4 or less; and removing the hydrophobic resin from the ADC mixture, such that the composition comprising ADCs is obtained, wherein the composition comprises less than 15% of the drug loaded species of 6 or more, and wherein the ADC comprises an antibody conjugated to a Bcl-xL inhibitor. In a separate embodiment, a batch method is used to contact an ADC mixture comprising a drug loaded species of 4 or less and a drug loaded species of 6 or more with a hydrophobic resin to form a resin mixture, wherein the amount of hydrophobic resin contacted with the ADC mixture is sufficient to allow binding of the drug loaded species of 6 or more to the resin but does not allow significant binding of the drug load species of 4 or less: and removing the hydrophobic resin from the ADC mixture, such that the composition comprising ADCs is obtained, wherein the composition comprises less than 15% of the drug loaded species of 6 or more, and wherein the ADC comprises an antibody conjugated to a Bcl-xL inhibitor, wherein the hydrophobic resin weight is 3 to 12 times the weight of the drug loaded species of 6 or more in the ADC mixture.

Alternatively, in a separate embodiment, purification may be performed using a circulation process, whereby the resin is packed in a container and the ADC mixture is passed over the hydrophobic resin bed until the specific species of ADC(s) to be separated have been removed. The supernatant (containing the desired ADC species) is then pumped from the container and the resin bed may be subjected to washing steps.

A circulation process may be used to contact an ADC mixture comprising a drug loaded species of 4 or less and a drug loaded species of 6 or more with a hydrophobic resin to form a resin mixture, wherein the amount of hydrophobic resin contacted with the ADC mixture is sufficient to allow binding of the drug loaded species of 6 or more to the resin but does not allow significant binding of the drug load species of 4 or less, and removing the hydrophobic resin from the ADC mixture, such that the composition comprising ADCs is obtained, wherein the composition comprises less than 15% of the drug loaded species of 6 or more, and wherein the ADC comprises an antibody conjugated to a Bcl-xL inhibitor. In a separate embodiment, a circulation process is used to contact an ADC mixture comprising a drug loaded species of 4 or less and a drug loaded species of 6 or more with a hydrophobic resin to form a resin mixture, wherein the amount of hydrophobic resin contacted with the ADC mixture is sufficient to allow binding of the drug loaded species of 6 or more to the resin but does not allow significant binding of the drug load species of 4 or less; and removing the hydrophobic resin from the ADC mixture, such that the composition comprising ADCs is obtained, wherein the composition comprises less than 15% of the drug loaded species of 6 or more, and wherein the ADC comprises an antibody conjugated to a Bcl-xL inhibitor, wherein the hydrophobic resin weight is 3 to 12 times the weight of the drug loaded species of 6 or more in the ADC mixture.

Alternatively, a flow through process may be used to purify an ADC mixture to arrive at a composition comprising a majority of ADCs having a certain desired DAR. In a flow through process, resin is packed in a container, e.g., a column, and the ADC mixture is passed over the packed resin such that the desired ADC species does not substantially bind to the resin and flows through the resin, and the undesired ADC species is bound to the resin. A flow through process may be performed in a single pass mode (where the ADC species of interest are obtained as a result of a single pass through the resin of the container) or in a multi-pass mode (where the ADC species of interest are obtained as a result of multiple passes through the resin of the container). The flow through process is performed such that the weight of resin selected binds to the undesired ADC population, and the desired ADCs (e.g., DAR 2-4) flow over the resin and are collected in the flow through after one or multiple passes.

A flow through process may be used to contact an ADC mixture comprising a drug loaded species of 4 or less and a drug loaded species of 6 or more with a hydrophobic resin, wherein the amount of hydrophobic resin contacted with the ADC mixture is sufficient to allow binding of the drug loaded species of 6 or more to the resin but does not allow significant binding of the drug load species of 4 or less, where the drug load species of 4 or less passes over the resin and is subsequently collected after one or multiple passes, such that the composition comprising the desired ADCs (e.g. DAR 2-4) is obtained, wherein the composition comprises less than 15% of the drug loaded species of 6 or more, and wherein the ADC comprises an antibody conjugated to a Bcl-xL inhibitor. In a separate embodiment, a flow through process is used to contact an ADC mixture comprising a drug loaded species of 4 or less and a drug loaded species of 6 or more with a hydrophobic resin by passing the ADC mixture over the resin, wherein the amount of hydrophobic resin contacted with the ADC mixture is sufficient to allow binding of the drug loaded species of 6 or more to the resin but does not allow significant binding of the drug load species of 4 or less, where the drug load species of 4 or less passes over the resin and is subsequently collected, such that the composition comprising ADCs is obtained, wherein the composition comprises less than 15% of the drug loaded species of 6 or more, and wherein the ADC comprises an antibody conjugated to a Bcl-xL inhibitor, wherein the amount of hydrophobic resin weight is 3 to 12 times the weight of the drug loaded species of 6 or more in the ADC mixture.

Following a flow through process, the resin may be washed with a one or more washes following in order to further recover ADCs having the desired DAR range (found in the wash filtrate). For example, a plurality of washes having decreasing conductivity may be used to further recover ADCs having the DAR of interest. The elution material obtained from the washing of the resin may be subsequently combined with the filtrate resulting from the flow through process for improved recovery of ADCs having the DAR of interest.

The aforementioned batch, circulation, and flow through process purification methods are based on the use of a hydrophobic resin to separate high vs. low drug loaded species of ADC. Hydrophobic resin comprises hydrophobic groups which interact with the hydrophobic properties of the ADCs. Hydrophobic groups on the ADC interact with hydrophobic groups within the hydrophobic resin. The more hydrophobic a protein is the stronger it will interact with the hydrophobic resin.

Hydrophobic resin normally comprises a base matrix (e.g., cross-linked agarose or synthetic copolymer material) to which hydrophobic ligands (e.g., alkyl or aryl groups) are coupled. Many hydrophobic resins are available commercially. Examples include, but are not limited to, Phenyl Sepharose™ 6 Fast Flow with low or high substitution (Pharmacia LKB Biotechnology, AB, Sweden); Phenyl Sepharose™ High Performance (Pharmacia LKB Biotechnology, AB, Sweden); Octyl Sepharose™ High Performance (Pharmacia LKB Biotechnology. AB. Sweden); Fractogel™ EMD Propyl or Fractogel™ EMD Phenyl columns (E. Merck, Germany); Macro-Prep™ Methyl or Macro-Prep™, t-Butyl Supports (Bio-Rad, California): WP HI-Propyl (C₃)™ (J. T. Baker, New Jersey); and Toyopearl™ ether, hexyl, phenyl or butyl (TosoHaas, PA). In one embodiment the hydrophobic resin is a butyl hydrophobic resin. In another embodiment, the hydrophobic resin is a phenyl hydrophobic resin. In another embodiment, the hydrophobic resin is a hexyl hydrophobic resin, an octyl hydrophobic resin, or a decyl hydrophobic resin. In one embodiment, the hydrophobic resin is a methacrylic polymer having n-butyl ligands (e.g. TOYOPEARL® Butyl-600M).

Further methods for purifying ADC mixtures to obtain a composition having a desired DAR are described in U.S. application Ser. No. 14/210,602 (U.S. Patent Appln. Publication No. US 2014/0286968), incorporated by reference in its entirety.

In certain embodiments of the invention, ADCs described herein having a DAR2 are purified from ADCs having higher or lower DARs. Such purified DAR2 ADCs are referred to herein as “E2”.

In certain embodiments of the invention, ADCs described herein having a DAR2 are purified from ADCs having higher or lower DARs. Such purified DAR2 ADCs are referred to herein as “E2”.

In one embodiment, the invention provides a composition comprising an ADC mixture, wherein at least 75% of the ADCs are anti-CD98 ADCs (like those described herein) having a DAR2. In another embodiment, the invention provides a composition comprising an ADC mixture, wherein at least 80% of the ADCs are anti-CD98 ADCs (like those described herein) having a DAR2. In another embodiment, the invention provides a composition comprising an ADC mixture, wherein at least 85% of the ADCs are anti-CD98 ADCs (like those described herein) having a DAR2. In another embodiment, the invention provides a composition comprising an ADC mixture, wherein at least 90% of the ADCs are anti-CD98 ADCs (like those described herein) having a DAR2.

### V. Uses of Anti-CD98 Antibodies and Anti-CD98 ADCs

The antibodies and antibody portions (and ADCs) of the invention preferably are capable of neutralizing human CD98 activity both in vivo. Accordingly, such antibodies and antibody portions of the invention can be used to inhibit hCD98 activity, e.g., in a cell culture containing hCD98, in human subjects or in other mammalian subjects having CD98 with which an antibody of the invention cross-reacts. In one embodiment, the invention provides a method for inhibiting hCD98 activity comprising contacting hCD98 with an antibody or antibody portion of the invention such that hCD98 activity is inhibited. For example, in a cell culture containing, or suspected of containing hCD98, an antibody or antibody portion of the invention can be added to the culture medium to inhibit hCD98 activity in the culture.

In another embodiment, of the invention a method for reducing hCD98 activity in a subject, advantageously from a subject suffering from a disease or disorder in which CD98 activity is detrimental. The invention provides methods for reducing CD98 activity in a subject suffering from such a disease or disorder, which method comprises administering to the subject an antibody or antibody portion of the invention such that CD98 activity in the subject is reduced. Preferably, the CD98 is human CD98, and the subject is a human subject. Alternatively, the subject can be a mammal expressing a CD98 to which antibodies of the invention are capable of binding. Still further the subject can be a mammal into which CD98 has been introduced (e.g., by administration of CD98 or by expression of a CD98 transgene). Antibodies of the invention can be administered to a human subject for therapeutic purposes. Moreover, antibodies of the invention can be administered to a non-human mammal expressing a CD98 with which the antibody is capable of binding for veterinary purposes or as an animal model of human disease. Regarding the latter, such animal models may be useful for evaluating the therapeutic efficacy of antibodies of the invention (e.g., testing of dosages and time courses of administration).

As used herein, the term “a disorder in which CD98 activity is detrimental” is intended to include diseases and other disorders in which the presence of CD98 in a subject suffering from the disorder has been shown to be or is suspected of being either responsible for the pathophysiology of the disorder or a factor that contributes to a worsening of the disorder. Accordingly, a disorder in which CD98 activity is detrimental is a disorder in which reduction of CD98 activity is expected to alleviate the symptoms and/or progression of the disorder. Such disorders may be evidenced, for example, by an increase in the concentration of CD98 in a biological fluid of a subject suffering from the disorder (e.g., an increase in the concentration of CD98 in a tumor, serum, plasma, synovial fluid, etc. of the subject), which can be detected, for example, using an anti-CD98 antibody as described above. Non-limiting examples of disorders that can be treated with the antibodies of the invention, for example, huAb102, huAb104, huAb108, or huAb110, or antigen binding fragments thereof, include those disorders discussed below. For example, suitable disorders include, but are not limited to, a variety of cancers including, but not limited to, breast cancer, lung cancer, a glioma, prostate cancer, pancreatic cancer, colon cancer, head and neck cancer, and kidney cancer. Other examples of cancer that may be treated using the compositions and methods disclosed herein include squamous cell carcinoma (e.g., squamous lung cancer or squamous head and neck cancer), triple negative breast cancer, non-small cell lung cancer, colorectal cancer, and mesothelioma. In one embodiment, the antibodies and ADCs disclosed herein are used to treat a solid tumor, e.g., inhibit growth of or decrease size of a solid tumor, overexpressing CD98 or which is CD98 positive. In one embodiment, the invention is directed to the treatment of CD98 amplified squamous lung cancer. In one embodiment, the antibodies and ADCs disclosed herein are used to treat CD98 amplified squamous head and neck cancer. In another embodiment, the antibodies and ADCs disclosed herein are used to treat triple negative breast cancer (TNBC). Diseases and disorders described herein may be treated by anti-CD98 antibodies or ADCs of the invention, as well as pharmaceutical compositions comprising such anti-CD98 antibodies or ADCs.

In certain embodiments, the antibodies and ADCs disclosed herein are administered to a subject in need thereof in order to treat advanced solid tumor types likely to exhibit elevated levels of CD98. Examples of such tumors include, but are not limited to, head and neck squamous cell carcinoma, non-small cell lung cancer, triple negative breast cancer, colorectal carcinoma, and glioblastoma multiforme.

In certain embodiments, the invention includes a method for inhibiting or decreasing solid tumor growth in a subject having a solid tumor, said method comprising administering an anti-CD98 antibody or ADC described herein, to the subject having the solid tumor, such that the solid tumor growth is inhibited or decreased. In certain embodiments, the solid tumor is a non-small cell lung carcinoma or a glioblastoma. In further embodiments, the solid tumor is an CD98 positive tumor or an CD98-expressing solid tumors. In further embodiments, the solid tumor is an CD98 amplified solid tumor or an CD98 overexpressing solid tumors. In certain embodiments the anti-CD98 antibodies or ADCs described herein are administered to a subject having glioblastoma multiforme, alone or in combination with an additional agent, e.g., radiation and/or temozolomide.

In certain embodiments, the invention includes a method for inhibiting or decreasing solid tumor growth in a subject having a solid tumor which was identified as an CD98 expressing or CD98 overexpressing tumor, said method comprising administering an anti-CD98 antibody or ADC described herein, to the subject having the solid tumor, such that the solid tumor growth is inhibited or decreased. Methods for identifying CD98 expressing tumors (e.g., CD98 overexpressing tumors) are known in the art, and include FDA-approved tests and validation assays. In addition. PCR-based assays may also be used for identifying CD98 overexpressing tumors. The amplified PCR products may be subsequently analyzed, for example, by gel electrophoresis using standard methods known in the art to determine the size of the PCR products. Such tests may be used to identify tumors that may be treated with the methods and compositions described herein.

Any of the methods for gene therapy available in the art can be used according to the invention. For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596: Mulligan, Science 260:926-932 (1993); and Morgan and Anderson, 1993, Ann. Rev. Biochem. 62:191-217: May, 1993, TIBITECH 11(5):155-215. Methods commonly known in the art of recombinant DNA technology which can be used are described in Ausubel et al. (eds.), Current Protocols in Molecular Biology, John Wiley & Sons, N Y (1993); and Kriegler, Gene Transfer and Expression, A Laboratory Manual, Stockton Press, NY (1990). Detailed description of various methods of gene therapy is provided in US20050042664 A1 which is incorporated herein by reference.

In another aspect, this application features a method of treating (e.g., curing, suppressing, ameliorating, delaying or preventing the onset of, or preventing recurrence or relapse of) or preventing a CD98-associated disorder, in a subject. The method includes: administering to the subject an CD98 binding agent (particularly an antagonist), e.g., an anti-CD98 antibody or fragment thereof as described herein, in an amount sufficient to treat or prevent the CD98-associated disorder. The CD98 antagonist, e.g., the anti-CD98 antibody or fragment thereof, can be administered to the subject, alone or in combination with other therapeutic modalities as described herein.

Antibodies or ADCs of the invention, or antigen binding portions thereof can be used alone or in combination to treat such diseases. It should be understood that the antibodies of the invention or antigen binding portion thereof can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the antibody of the invention. The additional agent also can be an agent that imparts a beneficial attribute to the therapeutic composition, e.g., an agent which affects the viscosity of the composition.

It should further be understood that the combinations which are to be included within this invention are those combinations useful for their intended purpose. The agents set forth below are illustrative for purposes and not intended to be limited. The combinations, which are part of this invention, can be the antibodies of the invention and at least one additional agent selected from the lists below. The combination can also include more than one additional agent, e.g., two or three additional agents if the combination is such that the formed composition can perform its intended function.

The combination therapy can include one or more CD98 antagonists, e.g., anti-CD98 antibodies or fragments thereof, formulated with, and/or co-administered with, one or more additional therapeutic agents, e.g., one or more cytokine and growth factor inhibitors, immunosuppressants, anti-inflammatory agents (e.g., systemic anti-inflammatory agents), anti-fibrotic agents, metabolic inhibitors, enzyme inhibitors, and/or cytotoxic or cytostatic agents, mitotic inhibitors, antitumor antibiotics, immunomodulating agents, vectors for gene therapy, alkylating agents, antiangiogenic agents, antimetabolites, boron-containing agents, chemoprotective agents, hormones, antihormone agents, corticosteroids, photoactive therapeutic agents, oligonucleotides, radionuclide agents, topoisomerase inhibitors, kinase inhibitors, or radiosensitizers, as described in more herein.

In a particular embodiment, the anti-CD98 binding proteins described herein, for example, anti-CD98 antibodies, are used in combination with an anti-cancer agent or an antineoplastic agent. The terms “anti-cancer agent” and “antineoplastic agent” refer to drugs used to treat malignancies, such as cancerous growths. Drug therapy may be used alone, or in combination with other treatments such as surgery or radiation therapy. Several classes of drugs may be used in cancer treatment, depending on the nature of the organ involved. For example, breast cancers are commonly stimulated by estrogens, and may be treated with drugs which inactive the sex hormones. Similarly, prostate cancer may be treated with drugs that inactivate androgens, the male sex hormone. Anti-cancer agents that may be used in conjunction with the anti-CD98 antibodies or ADCs of the invention include, among others, the following agents:

| Anti-Cancer Agent | Comments | Examples |
|---|---|---|
| Antibodies | Antibodies which bind IGF- | A12 (fully humanized mAb) |
| (a) antibodies other | 1R (insulin-like growth | 19D12 (fully humanized mAb) |
| than anti-CD98 | factor type 1 receptor), | Cp751-871 (fully humanized mAb) |
| antibodies | which is expressed on the | H7C10 (humanized mAb) |
| | cell surface of most human | alphaIR3 (mouse) |
| | cancers | ScFV/FC (mouse/human chimera) |
| | | EM/164 (mouse) |
| | Antibodies which bind | Matuzumab (EMD72000) |
| | CD98 (epidermal growth | Erbitux ®/Cetuximab (Imclone) |
| | factor receptor); Mutations | Vectibix ®/Pan itumumab (Amgen) |
| | affecting CD98 expression | mAb 806 |
| | or activity could result in | Nimotuxumab (TheraCIM) |
| | cancer | |
| | Antibodies which bind | AVEO (AV299) (AVEO) |
| | cMET (Mesechymal | AMG102 (Amgen) |
| | epithelial transition factor); | 5D5 (OA-5d5) (Genentech) |
| | a member of the MET | H244G11 (Pierre Fabre) |
| | family of receptor tyrosine | |
| | kinases) | |
| | Anti-ErbB3 | Ab #14 (MM 121-14) |
| | | Herceptin ® (Trastuzumab; Genentech) |
| | | 1B4C3; 2D1D12 (U3 Pharma AG) |
| Small Molecules | Insulin-like growth factor | NVP-AEW541-A |
| Targeting IGF1R | type 1 receptor which is | BMS-536,924 (1H-benzoimidazol-2-yl)-1H- |
| | expressed on the cell | pyridin-2-one) |
| | surface of many human | BMS-554,417 |
| | cancers | Cycloligan |
| | | TAE226 |
| | | PQ401 |
| Small Molecules | cMET (Mesenchymal | PHA665752 |
| Targeting cMET | epithelial transition factor); | ARQ 197 |
| | a member of the MET | |
| | family of receptor tyrosine | |
| | kinases) | |
| Antimetabolites | | Flourouracil (5-FU) |
| | | Capecitabine/XELODA ® (HLR Roche) |
| | | 5-Trifluoromethyl-2′-deoxyuridine |
| | | Methotrexate sodium (Trexall) (Barr) |
| | | Raltitrexed/Tomudex ® (AstraZeneca) |
| | | Pemetrexed/Alimta ® (Lilly) |
| | | Tegafur |
| | | Cytosine Arabinoside (Cytarabine, Ara-C)/ |
| | | Thioguanine ® (GlaxoSmithKline) |
| | | 5-azacytidine |
| | | 6-mercaptopurine (Mercaptopurine, 6-MP) |
| | | Azathioprine/Azasan ® (AAIPHARMA LLC) |
| | | 6-thioguanine (6-TG)/Purinethol ® (TEVA) |
| | | Pentostatin/Nipent ® (Hospira Inc.) |
| | | Fludarabine phosphate/Fludara ® (Bayer |
| | | Health Care) |
| | | Cladribine (2-CdA, 2-chlorodeoxyadenosine)/ |
| | | Leustatin ® (Ortho Biotech) |
| Alkylating agents | An alkydating antineoplastic | Ribonucleotide Reductase Inhibitor (RNR) |
| | agent is an alkylating agent | Cyclophosphamide/Cytoxan (BMS) |
| | that attaches an alkyl group | Neosar (TEVA) |
| | to DNA. Since cancer cells | Ifosfamide/Mitoxana ® (ASTA Medica) |
| | generally proliferate | Thiotepa (Bedford, Abraxis, Teva) |
| | unrestrictively more than do | BCNU→ 1,3-bis(2-chloroethyl)-1-nitosourea |
| | healthy cells they are more | CCNU→ 1,-(2-chloroethyl)-3-cyclohexyl-1- |
| | sensitive to DNA damage, | nitrosourea (methyl CCNU) |
| | and alkylating agents are | Hexamethylmelamine (Altretamine, HMM)/ |
| | used clinically to treat a | Hexalen ® (MGI Pharma Inc.) |
| | variety of tumors. | Busulfan/Myleran (GlaxoSmithKline) |
| | | Procarbazine HCL/Matulane (Sigma Tau |
| | | Pharmaceuticals, Inc.) |
| | | Dacarbazine (DTIC) |
| | | Chlorambucil/Leukara ® (SmithKline |
| | | Beecham) |
| | | Melphalan/Alkeran ® (GlaxoSmithKline) |
| | | Cisplatin (Cisplatinum, CDDP)/Platinol |
| | | (Bristol Myers) |
| | | Carboplatin/Paraplatin (BMS) |
| | | Oxaliplatin/Eloxitan ® (Sanofi-Aventis US) |
| Topoisomerase | Topoisomerase inhibitors | Doxorubicin HCL/Doxil ® (Alza) |
| inhibitors | are chemotherapy agents | Daunorubicin citrate/Daunoxome ® (Gilead) |
| | designed to interfere with | Mitoxantrone HCL/Novantrone (EMD |
| | the action of topoisomerase | Serono) |
| | enzymes (topoisomerase I | Actinomycin D |
| | and II), which are enzymes | Etoposide/Vepesid ® (BMS)/Etopophos ® |
| | that control the changes in | (Hospira, Bedford, Teva Parenteral, Etc.) |
| | DNA structure by | Topotecan HCL/Hycamtin ® |
| | catalyzing the breaking and | (GlaxoSmithKline) |
| | rejoining of the | Teniposide (VM-26)/Vumon ® (BMS) |
| | phosphodiester backbone of | Irinotecan HCL(CPT-ll)/Camptosar ® |
| | DNA strands during the | (Pharmacia & Upjohn) |
| | normal cell cycle. | |
| Microtubule | Microtubules are one of the | Vincristine/Oncovin ® (Lilly) |
| targeting agents | components of the | Vinblastine sulfate/Velban ®(discontinued) |
| | cytoskeleton. They have | (Lilly) |
| | diameter of ~24 nm and | Vinorelbine tartrate/Navelbine ®) |
| | length varying from several | (PierreFabre) |
| | micrometers to possibly | Vindesine sulphate/Eldisine ® (Lilly) |
| | millimeters in axons of | Paclitaxel/Taxol ® (BMS) |
| | nerve cells. Microtubules | Docetaxel/Taxotere ® (Sanofi Aventis US) |
| | serve as structural | Nanoparticle paclitaxel (ABI-007)/ |
| | components within cells and | Abraxane ® (Abraxis BioScience, Inc.) |
| | are involved in many | Ixabepilone/IXEMPRA ™ (BMS) |
| | cellular processes including | |
| | mitosis, cytokinesis, and | |
| | vesicular transport. | |
| Kinase inhibitors | Kinases are enzymes that | Imatinib mesylate/Gleevec (Novartis) |
| | catalyzes the transfer of | Sunitinib malate/Sutent ® (Pfizer) |
| | phosphate groups from | Sorafenib tosylate/Nexavar ® (Bayer) |
| | high-energy, phosphate- | Nilotinib hydrochloride monohydrate/ |
| | donating molecules to | Tasigna ® (Novartis), Osimertinib, |
| | specific substrates, and are | Cobimetinib, Trametinib, Dabrafenib, |
| | utilized to transmit signals | Dinaciclib |
| | and regulate complex | |
| | processes in cells. | |
| Protein synthesis | Induces cell apoptosis | L-asparaginase/Elspar ® (Merck & Co.) |
| inhibitors | | |
| Immunotherapeutic | Induces cancer patients to | Alpha interferon |
| agents | exhibit immune | Angiogenesis Inhibitor/Avastin ® |
| | responsiveness | (Genentech) |
| | | IL-2→ Interleukin 2 (Aldesleukin)/Proleukin ® |
| | | (Chiron) |
| | | IL-12→ Interleukin 12 |
| | Antibody/small molecule | Anti-CTLA-4 and PR-1 therapies |
| | immune checkpoint | Yervoy ® (ipilimumab; Bristol-Myers Squibb) |
| | modulators | Opdivo ® (nivolumab; Bristol-Myers Squibb) |
| | | Keytrada ® (pembrolizumab; Merck) |
| Hormones | Hormone therapies | Toremifene citrate/Fareston ® (GTX, Inc.) |
| | associated with menopause | Fulvestrant/Faslodex ® (AstraZeneca) |
| | and aging seek to increase | Raloxifene HCL/Evista ® (Lilly) |
| | the amount of certain | Anastrazole/Arimidex ® (AstraZeneca) |
| | hormones in your body to | Letrozole/Femara ® (Novartis) |
| | compensate for age- or | Fadrozole (CGS 16949A) |
| | disease-related hormonal | Exemestane/Aromasin ® (Pharmacia & |
| | declines. Hormone therapy | Upjohn) |
| | as a cancer treatment either | Leuprolide acetate/Eligard ® (QTL USA) |
| | reduces the level of specific | Lupron ® (TAP Pharm) |
| | hormones or alters the | Goserelin acetate/Zoladex ® (AstraZeneca) |
| | cancer's ability to use these | Triptorelin pamoate/Trelstar ® (Watson Labs) |
| | hormones to grow and | Buserelin/Suprefact ® (Sanofi Aventis) |
| | spread. | Nafarelin/Synarel ® (Pfizer) |
| | | Cetrorelix/Cetrotide ® (EMD Serono) |
| | | Bicalutamide/Casodex ® (AstraZeneca) |
| | | Nilutamide/Nilandron ® (Aventis Pharm.) |
| | | Megestrol acetate/Megace ® (BMS) |
| | | Somatostatin Analogs (Octreotide acetate/ |
| | | Sandostatin ® (Novartis) |
| Glucocorticoids | Anti-inflammatory drugs | Prednisolone |
| | used to reduce swelling that | Dexamethasone/Decadron ® (Wyeth) |
| | causes cancer pain. | |
| Aromatose inhibitors | Includes imidazoles | Ketoconazole |
| mTOR inhibitors | the mTOR signaling | Sirolimus (Rapamycin)/Rapamune ® (Wyeth) |
| | pathway was originally | Temsirolimus (CCI-779)/Torisel ® (Wyeth) |
| | discovered during studies of | Deforolimus (AP23573)/(Ariad Pharm.) |
| | the immunosuppressive | Everolimus (RAD00I)/Certican ® (Novartis) |
| | agent rapamycin. This | |
| | highly conserved pathway | |
| | regulates cell proliferation | |
| | and metabolism in response | |
| | to environmental factors, | |
| | linking cell growth factor | |
| | receptor signaling via | |
| | phosphoinositide-3- | |
| | kinase(PI-3K) to cell | |
| | growth, proliferation, and | |
| | angiogenesis. | |

In addition to the above anti-cancer agents, the anti-CD98 antibodies and ADCs described herein may be administered in combination with the agents described herein. Further, the aforementioned anti-cancer agents may also be used in the ADCs of the invention.

In particular embodiments, the anti-CD98 antibodies or ADCs can be administered alone or with another anti-cancer agent which acts in conjunction with or synergistically with the antibody to treat the disease associated with CD98 activity. Such anti-cancer agents include, for example, agents well known in the art (e.g., cytotoxins, chemotherapeutic agents, small molecules and radiation). Examples of anti-cancer agents include, but are not limited to, Panorex (Glaxo-Welcome), Rituxan (IDEC/Genentech/Hoffman la Roche), Mylotarg (Wyeth), Campath (Millennium), Zevalin (IDEC and Schering AG), Bexxar (Corixa/GSK), Erbitux (Imclone/BMS), Avastin (Genentech) and Herceptin (Genentech/Hoffman la Roche). Other anti-cancer agents include, but are not limited to, those disclosed in U.S. Pat. No. 7,598,028 and International Publication No, WO2008/100624, the contents of which are hereby incorporated by reference. One or more anti-cancer agents may be administered either simultaneously or before or after administration of an antibody or antigen binding portion thereof of the invention.

In particular embodiments of the invention, the anti-CD98 antibodies or ADCs described herein can be used in a combination therapy with an apoptotic agent, such as a Bcl-xL inhibitor or a Bcl-2 (B-cell lymphoma 2) inhibitor (e.g., ABT-199 (venetoclax)) to treat cancer, such as leukemia, in a subject. In one embodiment, the anti-CD98 antibodies or ADCs described herein can be used in a combination therapy with a Bcl-xL inhibitor for treating cancer. In one embodiment, the anti-CD98 antibodies or ADCs described herein can be used in a combination therapy with venetoclax for treating cancer.

In particular embodiments of the invention, the anti-CD98 antibodies or ADCs described herein can be used in a combination therapy with an inhibitor of NAMPT (see examples of inhibitors in US 2013/0303509; AbbVie. Inc., incorporated by reference herein) to treat a subject in need thereof. NAMPT (also known as pre-B-cell-colony-enhancing factor (PBEF) and visfatin) is an enzyme that catalyzes the phosphoribosylation of nicotinamide and is the rate-limiting enzyme in one of two pathways that salvage NAD. In one embodiment of the invention, anti-CD98 antibodies and ADCs described herein are administered in combination with a NAMPT inhibitor for the treatment of cancer in a subject.

In particular embodiments of the invention, the anti-CD98 antibodies or ADCs described herein can be used in a combination therapy with SN-38, which is the active metabolite of the topoisomerase inhibitor irinotecan.

In other embodiments of the invention, the anti-CD98 antibodies or ADCs described herein can be used in a combination therapy with a PARP (poly ADP ribose polymerase) inhibitor. e.g., veliparib, to treat cancer, including breast, ovarian and non-small cell lung cancers.

Further examples of additional therapeutic agents that can be co-administered and/or formulated with anti-CD98 antibodies or anti-CD98 ADCs described herein, include, but are not limited to, one or more of: inhaled steroids; beta-agonists, e.g., short-acting or long-acting beta-agonists; antagonists of leukotrienes or leukotriene receptors; combination drugs such as ADVAIR; IgE inhibitors, e.g., anti-IgE antibodies (e.g., XOLAIR®, omalizumnab); phosphodiesterase inhibitors (e.g., PDE4 inhibitors); xanthines; anticholinergic drugs; mast cell-stabilizing agents such as cromolyn; IL-4 inhibitors: IL-5 inhibitors; eotaxin/CCR3 inhibitors; antagonists of histamine or its receptors including H1, H2, H3, and H4, and antagonists of prostaglandin D or its receptors (DPI and CRTH2). Such combinations can be used to treat, for example, asthma and other respiratory disorders. Other examples of additional therapeutic agents that can be co-administered and/or formulated with anti-CD98 antibodies or anti-CD98 ADCs described herein, include, but are not limited to, one or more of, temozolomide, ibrutinib, duvelisib, and idelalisib. Additional examples of therapeutic agents that can be co-administered and/or formulated with one or more anti-CD98 antibodies or fragments thereof include one or more of: TNF antagonists (e.g., a soluble fragment of a TNF receptor, e.g., p55 or p75 human TNF receptor or derivatives thereof, e.g., 75 kD TNFR-IgG (75 kD TNF receptor-IgG fusion protein, ENBREL)); TNF enzyme antagonists, e.g., TNF converting enzyme (TACE) inhibitors; muscarinic receptor antagonists; TGF-beta antagonists; interferon gamma: perfenidone; chemotherapeutic agents, e.g., methotrexate, leflunomide, or a sirolimus (rapamycin) or an analog thereof, e.g., CCI-779; COX2 and cPLA2 inhibitors; NSAIDs; immunomodulators: p38 inhibitors, TPL-2, MK-2 and NFkB inhibitors, among others.

Other preferred combinations are cytokine suppressive anti-inflammatory drug(s) (CSAIDs); antibodies to or antagonists of other human cytokines or growth factors, for example, IL-, IL-2, IL-3. IL-4, IL-5, IL-6, IL-7, IL-8, IL-15, IL-16, IL-18, IL-21, IL-31, interferons, EMAP-II, GM-CSF, FGF. EGF, PDGF, and edothelin-1, as well as the receptors of these cytokines and growth factors. Antibodies of the invention, or antigen binding portions thereof, can be combined with antibodies to cell surface molecules such as CD2, CD3, CD4, CD8, CD25, CD28, CD30, CD40, CD45, CD69, CD80 (B7.1), CD86 (B7.2), CD90, CTLA, CTLA-4, PD-1, or their ligands including CD154 (gp39 or CD40L).

Preferred combinations of therapeutic agents may interfere at different points in the inflammatory cascade; preferred examples include TNF antagonists like chimeric, humanized or human TNF antibodies, adalimumab, (HUMIRA; D2E7; PCT Publication No, WO 97/29131 and U.S. Pat. No. 6,090,382, incorporated by reference herein), CA2 (REMICADE), CDP 571, and soluble p55 or p75 TNF receptors, derivatives, thereof, (p75TNFR1gG (ENBREL) or p55TNFR1gG (Lenercept), and also TNF converting enzyme (TACE) inhibitors; similarly IL-1 inhibitors (Interleukin-1-converting enzyme inhibitors, IL-1RA etc.) may be effective for the same reason. Other preferred combinations include Interleukin 4.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the antibody or antibody portion may be determined by a person skilled in the art and may vary according to factors such as the disease state, age, sex, and weight of the individual, and the ability of the antibody or antibody portion to elicit a desired response in the individual. A therapeutically effective amount is also one in which any toxic or detrimental effects of the antibody, or antibody portion, are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of an ADC, an antibody or antibody portion of the invention is 0.1-20 mg/kg, more preferably-10 mg/kg. In one embodiment, the dose of the antibodies and ADCs described herein is 1 to 6 mg/kg, including the individual doses recited therein, e.g., 1 mg/kg, 2 mg/kg, 3 mg/kg, 4 mg/kg, 5 mg/kg, and 6 mg/kg. In another embodiment, the dose of the antibodies and ADCs described herein is 1 to 200 g/kg, including the individual doses recited therein, e.g., 1 μg/kg, 2 μg/kg, 3 μg/kg, 4 μg/kg, 5 μg/kg, 10 μg/kg, 20 μg/kg, 30 μg/kg, 40 μg/kg, 50 μg/kg, 60 μg/kg, 80 μg/kg, 100 μg/kg, 120 μg/kg, 140 μg/kg, 160 μg/kg, 180 μg/kg and 200 μg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

In one embodiment, an anti-CD98 antibody described herein, e.g., huAb102, huAb104, huAb108, or huAb110, or an antigen binding portion thereof, is administered to a subject in need thereof, e.g., a subject having cancer, as an ADC at a dose of 0.1 to 30 mg/kg. In another embodiment, the anti-CD98 antibody, e.g., huAb102, huAb104, huAb108, or huAb110, or an antigen binding portion thereof, is administered to a subject in need thereof, e.g., a subject having cancer, as an ADC at a dose of 1 to 15 mg/kg. In another embodiment, the anti-CD98 antibody, e.g., huAb102, huAb104, huAb108, or huAb110, or an antigen binding portion thereof, is administered to a subject in need thereof, e.g., a subject having cancer, as an ADC at a dose of 1 to 10 mg/kg. In another embodiment, the anti-CD98 antibody, e.g., huAb102, huAb104, huAb108, or huAb110, or an antigen binding portion thereof, is administered to a subject in need thereof, e.g., a subject having cancer, as an ADC at a dose of 2 to 3. In another embodiment, the anti-CD98 antibody, e.g., HuAb102, huAb104, huAb108, or huAb110, or an antigen binding portion thereof, is administered to a subject in need thereof, e.g., a subject having cancer, as an ADC at a dose of 1 to 4 mg/kg.

In one embodiment, an anti-CD98 antibody described herein, e.g., huAb102, huAb104, huAb108, or huAb110, or an antigen binding portion thereof, is administered to a subject in need thereof, e.g., a subject having cancer, as an ADC at a dose of 1 to 200 μg/kg. In another embodiment, the anti-CD98 antibody, e.g., huAb102, huAb104, huAb108, or huAb110, or an antigen binding portion thereof, is administered to a subject in need thereof, e.g., a subject having cancer, as an ADC at a dose of 5 to 150 μg/kg. In another embodiment, the anti-CD98 antibody, e.g., huAb102, huAb104, huAb108, or huAb110, or an antigen binding portion thereof, is administered to a subject in need thereof, e.g., a subject having cancer, as an ADC at a dose of 5 to 100 μg/kg. In another embodiment, the anti-CD98 antibody, e.g., huAb102, huAb104, huAb108, or huAb110, or an antigen binding portion thereof, is administered to a subject in need thereof, e.g., a subject having cancer, as an ADC at a dose of 5 to 90 μg/kg. In another embodiment, the anti-CD98 antibody, e.g., huAb102, huAb104, huAb108, or huAb110, or an antigen binding portion thereof, is administered to a subject in need thereof, e.g., a subject having cancer, as an ADC at a dose of 5 to 80 μg/kg. In another embodiment, the anti-CD98 antibody, e.g., huAb102, huAb104, huAb108, or huAb110, or an antigen binding portion thereof, is administered to a subject in need thereof, e.g., a subject having cancer, as an ADC at a dose of 5 to 70 μg/kg. In another embodiment, the anti-CD98 antibody, e.g., huAb102, huAb104, huAb108, or huAb110, or an antigen binding portion thereof, is administered to a subject in need thereof, e.g., a subject having cancer, as an ADC at a dose of 5 to 60 μg/kg. In another embodiment, the anti-CD98 antibody, e.g., huAb102, huAb104, huAb108, or huAb110, or an antigen binding portion thereof, is administered to a subject in need thereof, e.g., a subject having cancer, as an ADC at a dose of 10 to 80 μg/kg.

In one embodiment, an anti-CD98 ADC described herein, e.g., huAb102-, huAb104-, huAb108-, or huAb110-vc-MMAE, is administered to a subject in need thereof, e.g., a subject having cancer, at a dose of 0.1 to 6 mg/kg. In another embodiment, an anti-CD98 ADC described herein, e.g., huAb102-, huAb104-, huAb108-, or huAb110-vc-MMAE, is administered to a subject in need thereof, e.g., a subject having cancer, at a dose of 0.5 to 4 mg/kg. In another embodiment, an anti-CD98 ADC described herein, e.g., huAb102-, huAb104-, huAb108-, or huAb110-vc-MMAE, is administered to a subject in need thereof, e.g., a subject having cancer, at a dose of 1.8 to 2.4 mg/kg. In another embodiment, an anti-CD98 ADC described herein, e.g., huAb102-, huAb104-, huAb108-, or huAb110-vc-MMAE, is administered to a subject in need thereof, e.g., a subject having cancer, at a dose of 1 to 4 mg/kg. In another embodiment, an anti-CD98 ADC described herein, e.g., huAb102-, huAb104-, huAb108-, or huAb110-vc-MMAE, is administered to a subject in need thereof, e.g., a subject having cancer, at a dose of about 1 mg/kg. In another embodiment, an anti-CD98 ADC described herein, e.g., huAb102-, huAb104-, huAb108-, or huAb110-vc-MMAE, is administered to a subject in need thereof, e.g., a subject having cancer, at a dose of 3 to 6 mg/kg. In another embodiment, an anti-CD98 ADC described herein, e.g., huAb102-, huAb104-, huAb108-, or huAb110-vc-MMAE, is administered to a subject in need thereof, e.g., a subject having cancer, at a dose of 3 mg/kg. In another embodiment, an anti-CD98 ADC described herein, e.g., huAb102-, huAb104-, huAb108-, or huAb10-vc-MMAE, is administered to a subject in need thereof, e.g., a subject having cancer, at a dose of 2 to 3 mg/kg. In another embodiment, an anti-CD98 ADC described herein, e.g., huAb102, huAb104, huAb108, or huAb110-vc-MMAE, is administered to a subject in need thereof, e.g., a subject having cancer, at a dose of 6 mg/kg.

In another embodiment, an anti-CD98 antibody described herein, conjugated to a drug, e.g., a PBD, (an ADC) is administered to a subject in need thereof, e.g., a subject having cancer, at a dose of 1 to 200 μg/kg. In another embodiment, an anti-CD98 ADC described herein, is administered to a subject in need thereof, e.g., a subject having cancer, at a dose of 5 to 100 μg/kg. In another embodiment, an anti-CD98 ADC described herein, is administered to a subject in need thereof, e.g., a subject having cancer, at a dose of 5 to 90 μg/kg. In another embodiment, an anti-CD98 ADC described herein, is administered to a subject in need thereof, e.g., a subject having cancer, at a dose of 5 to 80 μg/kg. In another embodiment, an anti-CD98 ADC described herein, is administered to a subject in need thereof, e.g., a subject having cancer, at a dose of 5 to 70 μg/kg. In another embodiment, an anti-CD98 ADC described herein, is administered to a subject in need thereof, e.g., a subject having cancer, at a dose of 5 to 60 μg/kg.

Doses described above may be useful for the administration of either anti-CD98 ADCs or antibodies disclosed herein.

In another aspect, this application provides a method for detecting the presence of CD98 in a sample in vitro (e.g., a biological sample, such as serum, plasma, tissue, biopsy). The subject method can be used to diagnose a disorder, e.g., a cancer. The method includes: (i) contacting the sample or a control sample with the anti-CD98 antibody or fragment thereof as described herein: and (ii) detecting formation of a complex between the anti-CD98 antibody or fragment thereof, and the sample or the control sample, wherein a statistically significant change in the formation of the complex in the sample relative to the control sample is indicative of the presence of CD98 in the sample.

Given their ability to bind to human CD98, the anti-human CD98 antibodies, or portions thereof, of the invention, (as well as ADCs thereof) can be used to detect human CD98 (e.g., in a biological sample, such as serum or plasma), using a conventional immunoassay, such as an enzyme linked immunosorbent assays (ELISA), an radioimmunoassay (RIA) or tissue immunohistochemistry. In one aspect, the invention provides a method for detecting human CD98 in a biological sample comprising contacting a biological sample with an antibody, or antibody portion, of the invention and detecting either the antibody (or antibody portion) bound to human CD98 or unbound antibody (or antibody portion), to thereby detect human CD98 in the biological sample. The antibody is directly or indirectly labeled with a detectable substance to facilitate detection of the bound or unbound antibody. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin: examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm.

Alternative to labeling the antibody, human CD98 can be assayed in biological fluids by a competition immunoassay utilizing rhCD98 standards labeled with a detectable substance and an unlabeled anti-human CD98 antibody. In this assay, the biological sample, the labeled rhCD98 standards and the anti-human CD98 antibody are combined and the amount of labeled rhCD98 standard bound to the unlabeled antibody is determined. The amount of human CD98 in the biological sample is inversely proportional to the amount of labeled rhCD98 standard bound to the anti-CD98 antibody. Similarly, human CD98 can also be assayed in biological fluids by a competition immunoassay utilizing rhCD98 standards labeled with a detectable substance and an unlabeled anti-human CD98 antibody.

In yet another aspect, this application provides a method for detecting the presence of CD98 in vivo (e.g., in vivo imaging in a subject). The subject method can be used to diagnose a disorder, e.g., a CD98-associated disorder. The method includes: (i) administering the anti-CD98 antibody or fragment thereof as described herein to a subject or a control subject under conditions that allow binding of the antibody or fragment to CD98; and (ii) detecting formation of a complex between the antibody or fragment and CD98, wherein a statistically significant change in the formation of the complex in the subject relative to the control subject is indicative of the presence of CD98

### VI. Pharmaceutical Compositions

The invention also provides pharmaceutical compositions comprising an antibody, or antigen binding portion thereof, or ADC of the invention and a pharmaceutically acceptable carrier. The pharmaceutical compositions comprising antibodies or ADCs of the invention are for use in, but not limited to, diagnosing, detecting, or monitoring a disorder, in preventing, treating, managing, or ameliorating of a disorder or one or more symptoms thereof, and/or in research. In a specific embodiment, a composition comprises one or more antibodies of the invention. In another embodiment, the pharmaceutical composition comprises one or more antibodies or ADCs of the invention and one or more prophylactic or therapeutic agents other than antibodies or ADCs of the invention for treating a disorder in which CD98 activity is detrimental. Preferably, the prophylactic or therapeutic agents known to be useful for or having been or currently being used in the prevention, treatment, management, or amelioration of a disorder or one or more symptoms thereof. In accordance with these embodiments, the composition may further comprise of a carrier, diluent or excipient.

The antibodies and antibody-portions or ADCs of the invention can be incorporated into pharmaceutical compositions suitable for administration to a subject. Typically, the pharmaceutical composition comprises an antibody or antibody portion of the invention and a pharmaceutically acceptable carrier. As used herein. “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody portion or ADC.

Various delivery systems are known and can be used to administer one or more antibodies or ADCs of the invention or the combination of one or more antibodies of the invention and a prophylactic agent or therapeutic agent useful for preventing, managing, treating, or ameliorating a disorder or one or more symptoms thereof, e.g., encapsulation in liposomes, microparticles, microcapsules, recombinant cells capable of expressing the antibody or antibody fragment, receptor-mediated endocytosis (see, e.g., Wu and Wu, J. Biol. Chem. 262:4429-4432 (1987)), construction of a nucleic acid as part of a retroviral or other vector, etc. Methods of administering a prophylactic or therapeutic agent of the invention include, but are not limited to, parenteral administration (e.g., intradermal, intramuscular, intraperitoneal, intravenous and subcutaneous), epidural administration, intratumoral administration, and mucosal administration (e.g., intranasal and oral routes). In addition, pulmonary administration can be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078: and PCT Publication Nos. WO 92/19244, WO 97/32572, WO 97/44013, WO 98/31346, and WO 99/66903, each of which is incorporated herein by reference their entireties. In one embodiment, an antibody of the invention, combination therapy, or a composition of the invention is administered using Alkermes AIR® pulmonary drug delivery technology (Alkermes, Inc., Cambridge. Mass.). In a specific embodiment, prophylactic or therapeutic agents of the invention are administered intramuscularly, intravenously, intratumorally, orally, intranasally, pulmonary, or subcutaneously. The prophylactic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the prophylactic or therapeutic agents of the invention locally to the area in need of treatment; this may be achieved by, for example, and not by way of limitation, local infusion, by injection, or by means of an implant, said implant being of a porous or non-porous material, including membranes and matrices, such as sialastic membranes, polymers, fibrous matrices (e.g., Tissuel®), or collagen matrices. In one embodiment, an effective amount of one or more antibodies of the invention antagonists is administered locally to the affected area to a subject to prevent, treat, manage, and/or ameliorate a disorder or a symptom thereof. In another embodiment, an effective amount of one or more antibodies of the invention is administered locally to the affected area in combination with an effective amount of one or more therapies (e.g., one or more prophylactic or therapeutic agents) other than an antibody of the invention of a subject to prevent, treat, manage, and/or ameliorate a disorder or one or more symptoms thereof.

In another embodiment, the prophylactic or therapeutic agent of the invention can be delivered in a controlled release or sustained release system. In one embodiment, a pump may be used to achieve controlled or sustained release (see Langer, supra; Sefton, 1987. CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the therapies of the invention (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. Nos. 5,679,377; 5,916,597; 5,912,015; 5,989,463; 5,128,326; PCT Publication No, WO 99/15154: and PCT Publication No, WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more therapeutic agents of the invention. See, e.g., U.S. Pat. No. 4,526,938, PCT publication WO 91/05548. PCT publication WO 96/20698. Ning et al., 1996, “Intratumoral Radioimmunotheraphy of a Human Colon Cancer Xenograft Using a Sustained-Release Gel.” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Sjmp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entireties.

In a specific embodiment, where the composition of the invention is a nucleic acid encoding a prophylactic or therapeutic agent, the nucleic acid can be administered in vive to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see, e.g., Joliot et al., 1991, Proc. Natl. Acad Sci. USA 88:1864-1868). Alternatively, a nucleic acid can be introduced intracellularly and incorporated within host cell DNA for expression by homologous recombination.

A pharmaceutical composition of the invention is formulated to be compatible with its intended route of administration. Examples of routes of administration include, but are not limited to, parenteral, e.g., intravenous, intradermal, subcutaneous, oral, intranasal (e.g., inhalation), transdermal (e.g., topical), transmucosal, and rectal administration. In a specific embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer. Where necessary, the composition may also include a solubilizing agent and a local anesthetic such as lignocaine to ease pain at the site of the injection.

If the method of the invention comprises intranasal administration of a composition, the composition can be formulated in an aerosol form, spray, mist or in the form of drops. In particular, prophylactic or therapeutic agents for use according to the invention can be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

If the method of the invention comprises oral administration, compositions can be formulated orally in the form of tablets, capsules, cachets, gel caps, solutions, suspensions, and the like. Tablets or capsules can be prepared by conventional means with pharmaceutically acceptable excipients such as binding agents (e.g., pregelatinized maize starch, poly vinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets may be coated by methods well-known in the art. Liquid preparations for oral administration may take the form of, but not limited to, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

The method of the invention may comprise pulmonary administration, e.g., by use of an inhaler or nebulizer, of a composition formulated with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entireties. In a specific embodiment, an antibody of the invention, combination therapy, and/or composition of the invention is administered using Alkermes AIR® pulmonary drug delivery technology (Alkermes, Inc., Cambridge, Mass.).

The method of the invention may comprise administration of a composition formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

The methods of the invention may additionally comprise of administration of compositions formulated as depot preparations. Such long acting formulations may be administered by implantation (e.g., subcutaneously or intramuscularly) or by intramuscular injection. Thus, for example, the compositions may be formulated with suitable polymeric or hydrophobic materials (e.g., as an emulsion in an acceptable oil) or ion exchange resins, or as sparingly soluble derivatives (e.g., as a sparingly soluble salt).

The methods of the invention encompass administration of compositions formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the mode of administration is infusion, composition can be dispensed with an infusion bottle containing sterile pharmaceutical grade water or saline. Where the mode of administration is by injection, an ampoule of sterile water for injection or saline can be provided so that the ingredients may be mixed prior to administration.

In particular, the invention also provides that one or more of the prophylactic or therapeutic agents, or pharmaceutical compositions of the invention is packaged in a hermetically sealed container such as an ampoule or sachette indicating the quantity of the agent. In one embodiment, one or more of the prophylactic or therapeutic agents, or pharmaceutical compositions of the invention is supplied as a dry sterilized lyophilized powder or water free concentrate in a hermetically sealed container and can be reconstituted (e.g., with water or saline) to the appropriate concentration for administration to a subject. Preferably, one or more of the prophylactic or therapeutic agents or pharmaceutical compositions of the invention is supplied as a dry sterile lyophilized powder in a hermetically scaled container at a unit dosage of at least 5 mg, at least 10 mg, at least 15 mg, at least 25 mg, at least 35 mg, at least 45 mg, at least 50 mg, at least 75 mg, or at least 100 mg. The lyophilized prophylactic or therapeutic agents or pharmaceutical compositions of the invention should be stored at between 2° C., and 8° C., in its original container and the prophylactic or therapeutic agents, or pharmaceutical compositions of the invention should be administered within 1 week, within 5 days, within 72 hours, within 48 hours, within 24 hours, within 12 hours, within 6 hours, within 5 hours, within 3 hours, or within 1 hour after being reconstituted. In an alternative embodiment, one or more of the prophylactic or therapeutic agents or pharmaceutical compositions of the invention is supplied in liquid form in a hermetically sealed container indicating the quantity and concentration of the agent. Preferably, the liquid form of the administered composition is supplied in a hermetically sealed container at least 0.25 mg/ml, at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml or at least 100 mg/ml. The liquid form should be stored at between 2° C., and 8° C., in its original container.

The antibodies and antibody-portions of the invention can be incorporated into a pharmaceutical composition suitable for parenteral administration. Preferably, the antibody or antibody-portions will be prepared as an injectable solution containing 0.1-250 mg/ml antibody. The injectable solution can be composed of either a liquid or lyophilized dosage form in a flint or amber vial, ampule or pre-filled syringe. The buffer can be L-histidine (1-50 mM), optimally 5-10 mM, at pH 5.0 to 7.0 (optimally pH 6.0). Other suitable buffers include but are not limited to, sodium succinate, sodium citrate, sodium phosphate or potassium phosphate. Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-methionine (optimally 5-10 mM). Other suitable bulking agents include glycine, arginine, can be included as 0-0.05% polysorbate-80 (optimally 0.005-0.01%). Additional surfactants include but are not limited to polysorbate 20 and BRIJ surfactants. The pharmaceutical composition comprising the antibodies and antibody-portions of the invention prepared as an injectable solution for parenteral administration, can further comprise an agent useful as an adjuvant, such as those used to increase the absorption, or dispersion of a therapeutic protein (e.g., antibody). A particularly useful adjuvant is hyaluronidase, such as Hylenex® (recombinant human hyaluronidase). Addition of hyaluronidase in the injectable solution improves human bioavailability following parenteral administration, particularly subcutaneous administration. It also allows for greater injection site volumes (i.e. greater than 1 ml) with less pain and discomfort, and minimum incidence of injection site reactions. (see WO2004078140, US2006104968 incorporated herein by reference).

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile, lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including, in the composition, an agent that delays absorption, for example, monostearate salts and gelatin.

The antibodies and antibody-portions or ADCs of the invention can be administered by a variety of methods known in the art, although for many therapeutic applications, the preferred route/mode of administration is subcutaneous injection, intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an antibody or antibody portion or ADC of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

In other embodiments, an antibody or antibody portion or ADC of the invention may be conjugated to a polymer-based species such that said polymer-based species may confer a sufficient size upon said antibody or antibody portion of the invention such that said antibody or antibody portion of the invention benefits from the enhanced permeability and retension effect (EPR effect) (See also PCT Publication No. WO2006/042146A2 and U.S. Publication Nos. 2004/0028687A1, 2009/0285757A1, and 2011/0217363A1, and U.S. Pat. No. 7,695,719 (each of which is incorporated by reference herein in its entirety and for all purposes).

Supplementary active compounds can also be incorporated into the compositions. In certain embodiments, an antibody or antibody portion or ADC of the invention is formulated with and/or co-administered with one or more additional therapeutic agents that are useful for treating disorders in which CD98 activity is detrimental. For example, an anti-hCD98 antibody or antibody portion or ADC of the invention may be formulated and/or co-administered with one or more additional antibodies that bind other targets (e.g., antibodies that bind cytokines or that bind cell surface molecules). Furthermore, one or more antibodies of the invention may be used in combination with two or more of the foregoing therapeutic agents. Such combination therapies may advantageously utilize lower dosages of the administered therapeutic agents, thus avoiding possible toxicities or complications associated with the various monotherapies.

In certain embodiments, an antibody or ADC to CD98 or fragment thereof is linked to a half-life extending vehicle known in the art. Such vehicles include, but are not limited to, the Fc domain, polyethylene glycol, and dextran. Such vehicles are described, e.g., in U.S. application Ser. No. 09/428,082 and published PCT Application No, WO 99/25044, which are hereby incorporated by reference for any purpose.

It will be readily apparent to those skilled in the art that other suitable modifications and adaptations of the methods of the invention described herein are obvious and may be made using suitable equivalents without departing from the scope of the invention or the embodiments disclosed herein. Having now described the invention in detail, the same will be more clearly understood by reference to the following examples, which are included for purposes of illustration only and are not intended to be limiting

### EXAMPLES

### Example 1. Synthesis of Exemplary Bcl-xL Inhibitors

This Example provides synthetic methods for exemplary Bcl-xL inhibitory compounds W3.01-W3.42. Bcl-xL inhibitors (W3.01-W3.43) and synthons (Examples 2.1-2.72) were named using ACD/Name 2012 release (Build 56084, 5 Apr. 2012, Advanced Chemistry Development Inc. Toronto, Ontario). ACD/Name 2014 release (Build 66687, 25 Oct. 2013. Advanced Chemistry Development Inc. Toronto. Ontario). ChemDraw® Ver. 9.0.7 (CambridgeSoft, Cambridge, Mass.), ChemDraw® Ultra Ver. 12.0 (CambridgeSoft, Cambridge, Mass.), or ChemDraw® Professional Ver. 15.0.0.106. Bcl-xL inhibitor and synthon intermediates were named with ACD/Name 2012 release (Build 56084, 5 Apr. 2012, Advanced Chemistry Development Inc. Toronto. Ontario). ACD/Name 2014 release (Build 66687, 25 Oct. 2013, Advanced Chemistry Development Inc., Toronto, Ontario). ChemDraw® Ver. 9.0.7 (CambridgeSoft, Cambridge, Mass.), ChemDraw® Ultra Ver. 12.0 (CambridgeSoft, Cambridge, Mass.), or ChemDraw® Professional Ver. 15.0.0.106.

### 1.1. Synthesis of 6-[1-(1,3-benzothiazol-2-yl)carbamoyl)-1,2,3,4-tetrahydroquinolin-7-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid (Compound W3.01)

### 1.1.1. 3-bromo-5,7-dimethyladamantanecarboxylic acid

To a 50 mL round-bottomed flask at 0° C., was added bromine (16 mL). Iron powder (7 g) was added, and the reaction was stirred at 0° C. for 30 minutes. 3,5-Dimethyladamantane-1-carboxylic acid (12 g) was then added. The mixture was then warmed to room temperature and stirred for 3 days. An ice/concentrated HCl mixture was poured into the reaction mixture. The resulting suspension was treated twice with Na₂SO₃ (50 g in 200 mL water) and extracted three times with dichloromethane. The combined organic layers were washed with 1N aqueous HCl, dried over Na₂SO₄, filtered, and concentrated to give the crude title compound.

### 1.1.2. 3-bromo-5,7-dimethyladamantanemethanol

To a solution of Example 1.1.1 (15.4 g) in tetrahydrofuran (200 mL) was added BH3 (1M in tetrahydrofuran, 150 mL). The mixture was stirred at room temperature overnight. The reaction mixture was then carefully quenched via dropwise addition of methanol. The mixture was then concentrated under vacuum and the residue was partitioned between ethyl acetate (500 mL) and 2N aqueous HCl (100 mL). The aqueous layer was further extracted twice with ethyl acetate and the combined organic extracts were combined and washed with water and brine, and dried over Na₂SO₄. Filtration and evaporation of the solvent gave the title compound.

### 1.1.3. 1-((3-bromo-5,7-dimethyltricyclo[3.3.1.1^{3,7}]dec-1-yl)methyl)-1H-pyrazole

To a solution of Example 1.1.2 (8.0 g) in toluene (60 mL) was added 1H-pyrazole (1.55 g) and cyanomethylenetributylphosphorane (2.0 g). The mixture was stirred at 90° C. overnight. The reaction mixture was then concentrated and the residue was purified by silica gel column chromatography (10:1 hexane:ethyl acetate) to provide the title compound. MS (ESI) m/e 324.2 (M+H)⁺.

### 1.1.4. 2-{[3,5-dimethyl-7-(1H-pyrazol-1-ylmethyl)tricyclo[3.3.1.1^{3,7}]dec-1-yl]oxy}ethanol

To a solution of Example 1.1.3 (4.0 g) in ethane-1,2-diol (12 mL) was added triethylamine (3 mL). The mixture was stirred at 150° C. under microwave conditions (Biotage) for 45 minutes. The mixture was poured into water (100 mL) and extracted three times with ethyl acetate. The combined organic extracts were washed with water and brine, and dried over Na₂SO₄. Filtration and evaporation of the solvent gave the crude title compound which was purified via column chromatography, eluting with 20% ethyl acetate in hexane followed by 5% methanol in dichloromethane, to provide the title compound. MS (ESI) m/e 305.2 (M+H)+.

### 1.1.5. 2-({3,5-dimethyl-7-[(5-methyl-1H-pyrazol-1-yl)methyl]tricyclo[3.3.1.1^{3,7}]dec-1-yl}oxy)ethanol

To a cooled (−78° C.) solution of Example 1.1.4 (6.05 g) in tetrahydrofuran (100 mL) was added n-BuLi (40 mL, 2.5M in hexane). The mixture was stirred at −78° C. for 1.5 hours. Then, iodomethane (10 mL) was added through a syringe and the mixture was stirred at −78° C. for 3 hours. The reaction mixture was then quenched with aqueous NH₄Cl and extracted twice with ethyl acetate, and the combined organic extracts were washed with water and brine. After drying over Na₂SO₄, the solution was filtered and concentrated and the residue was purified by silica gel column chromatography (5% methanol in dichloromethane) to provide the title compound. MS (ESI) m/e 319.5 (M+H)+.

### 1.1.6. 1-({3,5-dimethyl-7-[2-(hydroxy)ethoxy]tricyclo[3.3.1.1^{3,7}]dec-1-yl}methyl)-4-iodo-5-methyl-1H-pyrazole

To a solution of Example 1.1.5 (3.5 g) in N,N-dimethylformamide (30 mL) was added N-iodosuccinimide (3.2 g). The mixture was stirred at room temperature for 1.5 hours. The reaction mixture was then diluted with ethyl acetate (600 mL) and washed with aqueous NaHSO₃, water, and brine. After drying over Na2SO4, the solution was filtered and concentrated and the residue was purified by silica gel chromatography (20% ethyl acetate in dichloromethane) to give the title compound. MS (ESI) m/e 445.3 (M+H)+.

### 1.1.7. 2-((3-((4-iodo-5-methyl-1H-pyrazol-1-yl)methyl)-5,7-dimethyladamantan-1-yl)oxy)ethyl methanesulfonate

To a cooled solution (0° C.) of Example 1.1.6 (5.45 g) in dichloromethane (100 mL) was added triethylamine (5.13 mL) and methanesulfonyl chloride (0.956 mL). The mixture was stirred at room temperature for 1.5 hours, diluted with ethyl acetate (600) mL) and washed with water (120 mL) and brine (120 mL). The organic layer was dried over Na₂SO₄, filtered, and concentrated to provide the title compound. MS (ESI) m/e 523.4 (M+H)+.

### 1.1.8. 2-((3-((4-iodo-5-methyl-1H-pyrazol-1-yl)methyl)-5,7-dimethyladamantan-1-yl)oxy)-N-methylethanamine

A solution of Example 1.1.7 (6.41 g) in 2M methylamine in ethanol (15 mL) was stirred at overnight and concentrated. The residue was diluted with ethyl acetate and washed with aqueous NaHCO₃, water and brine. The organic layer was dried over Na₂SO₄, filtered, and concentrated to provide the title compound. MS (ESI) m/e 458.4 (M+H)+.

### 1.1.9, tert-butyl [2-({3-[(4-iodo-5-methyl-1H-pyrazol-1-yl)methyl]-5,7-dimethyltricyclo[3.3.1.1^{3,7}]dec-1-yl}oxy)ethyl]methylcarbamate

To a solution of Example 1.1.8 (2.2 g) in tetrahydrofuran (30 mL) was added di-tert-butyl dicarbonate (1.26 g) and a catalytic amount of 4-dimethylaminopyridine. The mixture was stirred at room temperature for 1.5 hours and then diluted with ethyl acetate (300 mL). The solution was washed with saturated aqueous NaHCO₃, water (60 mL) and brine (60 mL). The organic layer was dried with Na₂SO₄, filtered and concentrated. The residue was purified by silica gel chromatography, eluting with 20% ethyl acetate in dichloromethane, to provide the title compound. MS (ESI) m/e 558.5 (M+H)⁺.

### 1.1.10, tert-butyl (2-((3,5-dimethyl-7-((5-methyl-4-(4,4,5,5-tetramethyl-1,3,2-dioxaborolan-2-yl)-1H-pyrazol-1-yl)methyl)adamantan-1-yl)oxy)ethyl)(methyl)carbamate

To a solution of Example 1.1.9 (1.2 g) in dioxane was added bis(benzonitrile)palladium(II) chloride (0.04 g), 4,4,5,5-tetramethyl-1,3,2-dioxaborolane (0.937 mL) and triethylamine (0.9 mL). The mixture was heated at reflux overnight, diluted with ethyl acetate and washed with water (60 mL) and brine (60 mL). The organic layer was dried over Na₂SO₄, filtered and concentrated to provide the title compound. MS (ESI) m/e 558.5 (M+H)⁺.

### 1.1.11, tert-butyl 3-(1-((3-(2-((tert-butoxycarbonyl)(methyl)amino) ethoxy)-5,7-dimethyladamantan-1-yl)methyl)-5-methyl-1H-pyrazol-4-yl)-6-chloropicolinate

To Example 1.1.10 (100 mg) and tert-butyl 3-bromo-6-chloropicolinate (52.5 mg) in dioxane (2 mL) was added tris(dibenzylideneacetone)dipalladium(0) (8.2 mg), K₃PO₄ (114 mg), 1,3,5,7-tetramethyl-8-phenyl-2,4,6-trioxa-8-phosphaadamantane (5.24 mg) and water (0.8 mL). The mixture was stirred at 95° C. for 4 hours, diluted with ethyl acetate and washed with water and brine. The organic layer was dried over Na₂SO₄, filtered, concentrated and purified by flash chromatography, eluting with 20% ethyl acetate in heptanes and then with 5% methanol in dichloromethane, to provide the title compound. MS (ESI) m/e 643.3 (M+H)⁺.

### 1.1.12, tert-butyl 3-(1-((3-(2-((tert-butoxycarbonyl)(methyl)amino)ethoxy)-5,7-dimethyladamantan-1-yl)methyl)-5-methyl-1H-pyrazol-4-yl)-6-(1,2,3,4-tetrahydroquinolin-7-yl)picolinate

A mixture of Example 1.1.11 (480 mg), 7-(4,4,5,5-tetramethyl-1,3,2-dioxaborolan-2-yl)-1,2,3,4-tetrahydroquinoline (387 mg), dichlorobis(triphenylphosphine)-palladium(II) (78 mg) and CsF (340 mg) in dioxane (12 mL) and water (5 mL) was heated at 100° C. for 5 hours. After this time the reaction mixture was allowed to cool to room temperature and then diluted with ethyl acetate. The resulting mixture was washed with water and brine, and the organic layer was dried over Na₂SO₄, filtered, and concentrated. The residue was purified by flash chromatography, eluting with 50% ethyl acetate in heptanes to provide the title compound. MS (APCI) m/e 740.4 (M+H)⁺.

### 1.1.13, tert-butyl 6-(1-(benzo[d]thiazol-2-ylcarbamoyl)-1,2,3,4-tetrahydroquinolin-7-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

To a solution of benzo[d]thiazol-2-amine (114 mg) in acetonitrile (5 mL) was added bis(2,5-dioxopyrrolidin-1-yl) carbonate (194 mg). The mixture was stirred for 1 hour, and Example 1.1.12 (432 mg) in acetonitrile (5 mL) was added. The mixture was stirred overnight diluted with ethyl acetate, washed with water and brine, and the organic layer was dried over Na₂SO₄, filtered, and concentrated. The residue was purified by flash chromatography, eluting with 50% ethyl acetate in heptanes to provide the title compound.

### 1.1.14. 6-(1-(benzo[d]thiazol-2-ylcarbamoyl)-1,2,3,4-tetrahydroquinolin-7-yl)-3-(1-((3,5-dimethyl-7-(2-(methylamino)ethoxy)adamantan-1-yl)methyl)-5-methyl-1H-pyrazol-4-yl)picolinic acid

Example 1.1.13 (200 mg) in dichloromethane (5 mL) was treated with trifluoroacetic acid (2.5 mL) overnight. The mixture was concentrated to provide the title compound. ¹H NMR (400 MHz, dimethyl sulfoxide-d₆) δ ppm 8.40 (s, 1H), 8.30 (s, 2H), 8.02 (d, 1H), 7.85 (d, 1H), 7.74-7.83 (m, 2H), 7.42-7.53 (m, 2H), 7.38 (t, 1H), 7.30 (d, 1H), 7.23 (t, 1H), 3.93-4.05 (m, 2H), 3.52-3.62 (m, 2H), 2.97-3.10 (m, 2H), 2.84 (t, 2H), 2.56 (t, 2H), 2.23 (s, 3H), 1.88-2.00 (m, 2H), 1.45 (s, 2H), 1.25-1.39 (m, 4H), 1.12-1.22 (m, 4H), 1.00-1.09 (m, 2H), 0.89 (s, 6H). MS (ESI) m/e 760.1 (M+H)⁺.

### 1.2. Synthesis of 6-[4-(1,3-benzothiazol-2-ylcarbamoyl)-3,4-dihydro-2H-1,4-benzoxazin-6-yl]-3-[1-((3,5-dimethyl-7-[2-(methylamino)ethoxy]tricyclo[3.3.1.1^{3,7}]dec-1-yl)methyl)-5-methyl-1H-pyrazol-4-yl]pyridine-2-carboxylic acid (Compound W3.02)

### 1.2.1, tert-butyl 3-(1-(((-3-(2-((tert-butoxycarbonyl)(methyl) amino)ethoxy)-5,7-dimethyladamantan-1-yl)methyl)-5-methyl-1H-pyrazol-4-yl)-6-(3,4-dihydro-2H-benzo[b] [1,4]oxazin-6-yl)picolinate

To a solution of 6-(4.4,5,5-tetramethyl-1,3,2-dioxaborolan-2-yl)-3,4-dihydro-2H-benzo[b][1,4]oxazine (122 mg) in dioxane (4 mL) and water (1 mL) was added Example 1.1.11 (300 mg), bis(triphenylphosphine)palladium(II) dichloride (32.7 mg), and CsF (212 mg). The mixture was stirred at reflux overnight. The mixture was diluted with ethyl acetate (500 mL) and washed with water, brine and dried over Na₂SO₄. Filtration and evaporation of the solvents gave crude material which was purified via column chromatography (20% ethyl acetate in heptane followed by 5% methanol in dichloromethane) to provide the title compound. MS (ESI) m e 742.4 (M+H)⁺.

### 1.2.2. 6-[4-(1,3-benzothiazol-2-ylcarbamoyl)-3,4-dihydro-2H-1,4-benzoxazin-6-yl]-3-[1-((3,5-dimethyl-7-[2-(methylamino)ethoxy]tricyclo[3.3.1.1^{3,7}]dec-1-yl)methyl)-5-methyl-1H-pyrazol-4-yl]pyridine-2-carboxylic acid

To an ambient suspension of bis(2,5-dioxopyrrolidin-1-yl) carbonate (70.4 mg) in acetonitrile (4 mL) was added benzo[d]thiazol-2-amine (41.3 mg) and the mixture was stirred for one hour. A solution of Example 1.2.1 (170 mg) in acetonitrile (1 mL) and water (10 mL) was added, and the suspension was stirred vigorously overnight. The mixture was diluted with ethyl acetate (500 mL) and washed with water, brine and dried over Na₂SO₄. Filtration and evaporation of the solvents afforded a residue which was loaded on a column and eluted with 20% ethyl acetate in heptane followed by 5% methanol in dichloromethane. The resultant material was treated with 20% TFA in dichloromethane overnight. After evaporation of the solvent, the residue was purified via HPLC (Gilson system, eluting with 10-85% acetonitrile in 0.1% TFA in water) to provide the title compound. ¹H NMR (400 MHz, dimethyl sulfoxide-d₆) δ ppm 8.76 (s, 1H), 8.24-8.46 (m, 2H), 7.97 (d, 1H), 7.70-7.89 (m, 3H), 7.47 (s, 1H), 7.35-7.47 (m, 2H), 7.24 (t, 1H), 7.02 (d, 1H), 4.32-4.42 (m, 3H), 4.14-4.23 (m, 3H), 3.90 (s, 3H), 3.57 (t, 3H), 2.93-3.11 (m, 2H), 2.57 (t, 3H), 2.23 (s, 3H), 1.46 (s, 2H), 1.24-1.39 (m, 4H), 0.98-1.25 (m, 5H), 0.89 (s, 6H). MS (ESI) m/e 760.4 (M+H)⁺.

### 1.3. Synthesis of 6-[4-(1,3-benzothiazol-2-ylcarbamoyl)-1-methyl-1,2,3,4-tetrahydroquinoxalin-6-yl]-3-[1-((3,5-dimethyl-7-[2-(methylamino)ethoxy]tricyclo[3.3.1.1^{3,7}]dec-1-yl)methyl)-5-methyl-1H-pyrazol-4-yl]pyridine-2-carboxylic acid (Compound W3.03)

### 1.3.1, tert-butyl 3-(1-((3-(2-((tert-butoxycarbonyl)(methyl)amino) ethoxy)-5,7-dimethyladamantan-1-yl)methyl)-5-methyl-1H-pyrazol-4-yl)-6-(1-methyl-1,2,3,4-tetrahydroquinoxalin-6-yl)picolinate

To a solution of 1-methyl-6-(4,4,5,5-tetramethyl-1,3,2-dioxaborolan-2-yl)-1,2,3,4-tetrahydroquinoxaline (140 mg) in dioxane (4 mL) and water (1 mL) was added Example 1.1.11 (328 mg), bis(triphenylphosphine)palladium(II) dichloride (35.8 mg), and CsF (232 mg). The mixture was stirred at reflux overnight. The mixture was diluted with ethyl acetate (500 mL) and washed with water, brine and dried over Na₂SO₄. Filtration and evaporation of the solvent gave crude material which was purified via column chromatography, eluting with 20% ethyl acetate in heptane followed by 5% methanol in dichloromethane, to provide the title compound. MS (ESI) m/e 755.5 (M+H)⁺.

### 1.3.2. 6-[4-(1,3-benzothiazol-2-ylcarbamoyl)-1-methyl-1,2,3,4-tetrahydroquinoxalin-6-yl]-3-[1-(3,5-dimethyl-7-[2-(methylamino)ethoxy]tricyclo[3.3.1.1^{3,7}]dec-1-yl)methyl)-5-methyl-1H-pyrazol-4-yl]pyridine-2-carboxylic acid

To an ambient suspension of bis(2,5-dioxopyrrolidin-1-yl) carbonate (307 mg) in acetonitrile (10 mL) was added benzo[d]thiazol-2-amine (180 mg) and the mixture was stirred for one hour. A solution of Example 1.3.1 (600 mg) in acetonitrile (3 mL) was added, and the suspension was vigorously stirred overnight. The mixture was diluted with ethyl acetate (500 mL) and washed with water and brine and dried over Na₂SO₄. Filtration and evaporation of the solvents afforded a residue which was loaded on a column and eluted with 20% ethyl acetate in heptane (1 L) followed by 5% methanol in dichloromethane. The resultant material was treated with 20% TFA in dichloromethane overnight. After evaporation of solvent, the residue was purified on an HPLC (Gilson system, eluting with 10-85% acetonitrile in 0.1% TFA in water) to give the title compound. ¹H NMR (400 MHz, dimethyl sulfoxide-d₆) δ ppm 8.17-8.44 (m, 3H), 7.90 (d, 1H), 7.68-7.84 (m, 3H), 7.45 (s, 2H), 7.37 (t, 1H), 7.22 (t, 1H), 6.83 (d, 1H), 3.96-4.12 (m, 2H), 3.89 (s, 3H), 3.57 (t, 2H), 3.44 (t, 2H), 2.93-3.09 (m, 4H), 2.56 (t, 3H), 2.21 (s, 3H), 1.45 (s, 2H), 1.25-1.39 (m, 4H), 0.99-1.22 (m, 7H), 0.89 (s, 6H)⁺. MS (ESI) m/e 760.4 (M+H)⁺.

### 1.4. Synthesis of 3-(1-{[3-(2-aminoethoxy)-5,7-dimethyltricyclo[3.3.1.1^{3,7}]dec-1-yl]methyl}-5-methyl-1H-pyrazol-4-yl)-6-[1-(1,3-benzothiazol-2-ylcarbamoyl)-5,6-dihydroimidazo[1,5-a]pyrazin-7(8H)-yl]pyridine-2-carboxylic acid (Compound W3.04)

### 1.4.1. 2-((3-((4-iodo-5-methyl-1H-pyrazol-1-yl)methyl)-5,7-dimethyladamantan-1-yl)oxy)ethanamine

A solution of Example 1.1.7 (4.5 g) in 7N ammonium in methanol (15 mL) was stirred at 100° C. for 20 minutes under microwave conditions (Biotage Initiator). The reaction mixture was concentrated under vacuum. The residue was diluted with ethyl acetate (400 mL) and washed with aqueous NaHCO₃, water (60 mL) and brine (60 mL). The organic layer was dried (anhydrous Na₂SO₄), the solution was filtered and concentrated, and the residue was used in the next reaction without further purification. MS (ESI) m/e 444.2 (M+H)⁺.

### 1.4.2, tert-butyl (2-((3-((4-iodo-5-methyl-1H-pyrazol-1-yl)methyl)-5,7-dimethyladamantan-1-yl)oxy)ethyl)carbamate

To a solution of Example 1.4.1 (4.4 g) in tetrahydrofuran (100 mL) was added di-tert-butyl dicarbonate (2.6 g) and N,N-dimethyl-4-aminopyridine (100 mg). The mixture was stirred for 1.5 hours. The reaction mixture was diluted with ethyl acetate (300 mL) and washed with aqueous NaHCO₃, water (60 mL) and brine (60 mL). After drying (anhydrous Na₂SO₄), the solution was filtered and concentrated, and the residue was purified by silica gel column chromatography (20% ethyl acetate in dichloromethane) to give the title compound. MS (ESI) m/e 544.2 (M+H)⁺.

### 1.4.3. 6-fluoro-3-bromopicolinic acid

A slurry of 6-amino-3-bromopicolinic acid (25 g) in 400 mL 1:1 dichloromethane/chloroform was added to nitrosonium tetrafluoroborate (18.2 g) in dichloromethane (100 mL) at 5° C. over 1 hour. The resulting mixture was stirred for another 30 minutes, warmed to 35° C., and stirred overnight. The reaction mixture was cooled to room temperature and adjusted to pH 4 with a NaH₂PO₄ solution. The resulting solution was extracted three times with dichloromethane, and the combined extracts were washed with brine, dried over sodium sulfate, filtered and concentrated to provide the title compound.

### 1.4.4. Tert-butyl 3-bromo-6-fluoropicolinate

Para-toluenesulfonyl chloride (27.6 g) was added to a solution of Example 1.4.3 (14.5 g), pyridine (26.7 mL) and tert-butanol (80 mL) in dichloromethane (100 mL) at 0° C. The reaction was stirred for 15 minutes, warmed to room temperature, and stirred overnight. The solution was concentrated and partitioned between ethyl acetate and Na₂CO₃ solution. The layers were separated, and the aqueous layer was extracted with ethyl acetate. The organic layers were combined, rinsed with Na₂CO₃ solution and brine, dried over sodium sulfate, filtered, and concentrated to provide the title compound.

### 1.4.5. Ethyl 7-(5-bromo-6-(tert-butoxycarbonyl)pyridin-2-yl)-5,6,7,8-tetrahydroimidazo[1,5-a]pyrazine-1-carboxylate

Ethyl 5,6,7,8-tetrahydroimidazo[1,5-a]pyrazine-1-carboxylate hydrochloride (692 mg) and Example 1.4.4 (750 mg) were dissolved in dimethyl sulfoxide (6 mL). N,N-Diisopropylethylamine (1.2 mL) was added, and the solution was heated at 50° C. for 16 hours. The solution was cooled, diluted with water (20 mL), and extracted with ethyl acetate (50 mL). The organic portion was washed with brine and dried on anhydrous sodium sulfate. The solution was concentrated and, upon standing for 16 hours, solid crystals formed. The crystals were washed with diethyl ether to yield the title compound. MS (ESI) m/e 451. 453 (M+H)⁺, 395, 397 (M-tert-butyl)⁺.

### 1.4.6. Ethyl 7-(6-(tert-butoxycarbonyl)-5-(4,4,5,5-tetramethyl-1,3,2-dioxaborolan-2-yl)pyridin-2-yl)-5,6,7,8-tetrahydroimidazo[1,5-a]pyrazine-1-carboxylate

The title compound was prepared by substituting Example 1.4.5 for Example 1.1.9 in Example 1.1.10. MS (ESI) m/e 499 (M+H)⁺, 443 (M-tert-butyl)+, 529 (M+MeOH−H)⁻.

### 1.4.7. Ethyl 7-(6-(tert-butoxycarbonyl)-5-(1-((3-(2-((tert-butoxycarbonyl)amino)ethoxy)-5,7-dimethyladamantan-1-yl)methyl)-5-methyl-1H-pyrazol-4-yl)pyridin-2-yl)-5,6,7,8-tetrahydroimidazo[1,5-a]pyrazine-1-carboxylate

Example 1.4.6 (136 mg) and Example 1.4.2 (148 mg) were dissolved in 1,4-dioxane (3 mL) and water (0.85 mL). Tripotassium phosphate (290 mg) was added, and the solution was degassed and flushed with nitrogen three times. Tris(dibenzylideneacetone)dipalladium(0) (13 mg) and 1,3,5,7-tetramethyl-8-tetradecyl-2,4,6-trioxa-8-phosphaadamantane (12 mg) were added. The solution was degassed, flushed with nitrogen once, and heated to 70° C. for 16 hours. The reaction was cooled and diluted with ethyl acetate (10 mL) and water (3 mL). The layers were separated, and the organic layer was washed with brine and dried on anhydrous sodium sulfate. After filtration, the filtrate was concentrated and purified by flash column chromatography on silica gel, eluting with 5% methanol in ethyl acetate. The solvent was removed under reduced pressure to give the title compound. MS (ESI) m/e 760 (M+H)⁺, 758 (M−H)⁻.

### 1.4.8. 7-(6-(tert-butoxycarbonyl)-5-(1-((3-(2-((tert-butoxycarbonyl)amino)ethoxy)-5,7-dimethyladamantan-1-yl)methyl)-5-methyl-1H-pyrazol-4-yl)pyridin-2-yl)-5,6,7,8-tetrahydroimidazo[1,5-a]pyrazine-1-carboxylic acid

Example 1.4.7 (200 mg) was dissolved in tetrahydrofuran (0.7 mL), methanol (0.35 mL), and water (0.35 mL). Lithium hydroxide monohydrate (21 mg) was added, and the solution was stirred at room temperature for 16 hours. HCl (1M, 0.48 mL) was added and the water was removed by azeotroping twice with ethyl acetate (20 mL). The solvent was removed under reduced pressure, and the material was dried under vacuum. The material was dissolved in dichloromethane (5 mL) and ethyl acetate (1 mL) and dried over anhydrous sodium sulfate. After filtration, the solvent was removed under reduced pressure to give the title compound. MS (EST) me 760 (M+H)⁺, 758 (M−H)⁻.

### 1.4.9. Tert-butyl 6-(1-(benzo[d]thiazol-2-ylcarbamoyl)-5,6-dihydroimidazo[1,5-a]pyrazin-7(8H)-yl)-3-(1-((3-(2-((tert-butoxycarbonyl)amino)ethoxy)-5,7-dimethyladamantan-1-yl)methyl)-5-methyl-1H-pyrazol-4-yl)picolinate

Example 1.4.8 (160 mg) and benzo[d]thiazol-2-amine (35 mg) were dissolved in dichloromethane (1.5 mL). 1-Ethyl-3-[3-(dimethylamino)propyl]-carbodiimide hydrochloride (85 mg) and 4-(dimethylamino)pyridine (54 mg) were added, and the solution was stirred at room temperature for 16 hours. The material was purified by flash column chromatography on silica gel, eluting with 2.5-5% methanol in ethyl acetate. The solvent was removed under reduced pressure to give the title compound. MS (ESI) m/e 892 (M+H)⁺, 890 (M−H)⁻.

### 1.4.10. 3-(1-{[3-(2-aminoethoxy)-5,7-dimethyltricyclo[3.3.1.1^{3,7}]dec-1-yl]methyl}-5-methyl-1H-pyrazol-4-yl)-6-[1-(1,3-benzothiazol-2-ylcarbamoyl)-5,6-dihydroimidazo[1,5-a]pyrazin-7(8H)-yl]pyridine-2-carboxylic acid

The title compound was prepared by substituting Example 1.4.9 for Example 1.1.13 in Example 1.1.14. ¹H NMR (400 MHz, dimethyl sulfoxide-d₆) δ ppm 11.50 (bs, 1H), 8.21 (d, 1H), 7.98 (d, 1H), 7.93 (s, 1H), 7.76 (d, 1H), 7.66 (bs, 3H), 7.58 (d, 1H), 7.44 (t, 1H), 7.33 (s, 1H), 7.31 (t, 1H), 7.15 (d, 1H), 6.97 (d, 1H), 5.10 (s, 2H), 4.26 (m, 2H), 4.08 (t, 2H), 3.84 (s, 2H), 2.90 (m, 4H), 2.13 (s, 3H), 1.42 (s, 2H), 1.30 (q, 4H), 1.15 (m, 2H), 1.04 (q, 4H), 0.87 (s, 6H). MS (ESI) m/e 736 (M+H)⁺, 734 (M−H)⁻.

### 1.5. Synthesis of 3-(1-{[3-(2-aminoethoxy)-5,7-dimethyltricyclo[3.3.1.1′⁷]dec-1-yl]methyl}-5-methyl-1H-pyrazol-4-yl)-6-[8-(1,3-benzothiazol-2-ylcarbamoyl)-5-hydroxy-3,4-dihydroisoquinolin-2(1H)-yl]pyridine-2-carboxylic acid (Compound W3.05)

### 1.5.1, tert-butyldiphenyl(vinyl)silane

The title compound was prepared as described in J Org Chem. 70(4), 1467 (2005).

### 1.5.2. 2-(tert-butyldiphenylsilyl)ethanol

Example 1.5.1 (8.2 g) was dissolved in tetrahydrofuran (30 mL), then a 0.5M solution of 9-borabicyclo[3.3.1]nonane in tetrahydrofuran (63 mL) was added and the reaction was stirred at room temperature for 2.5 hours. The reaction was warmed to 37° C., then 3.0N aqueous NaOH (11 mL) was added, followed by the very careful dropwise addition of 30% aqueous H₂O₂(11 mL). Once the peroxide addition was completed, the reaction was stirred for one hour, and water (200 mL) and diethyl ether (200 mL) were added. The organic layer was washed with brine and dried over sodium sulfate. After filtration and concentration, purification by silica gel chromatography, eluting with heptanes/ethyl acetate (3/1), gave the title compound.

### 1.5.3. 5-(2-(tert-butyldiphenylsilyl)ethoxy)isoquinolne

Triphenylphosphine (262 mg) was dissolved in tetrahydrofuran (2 mL). Example 1.5.2 (285 mg), isoquinolin-5-ol (121 mg), and diisopropyl azodicarboxylate (203 mg) were added. The reaction was stirred at room temperature for 30 minutes, then more isoquinolin-5-ol (41 mg) was added and the reaction was stirred overnight. The reaction was then concentrated and purification by flash chromatography, eluting with heptanes/ethyl acetate (83/17), gave the title compound. MS (DCI) m/e 412.2 (M+H)⁺.

### 1.5.4. 8-bromo-5-(2-(tert-butyldiphenylsilyl)ethoxy)isoquinoline

Example 1.5.3 (6.2 g) was dissolved in acetic acid (40 mL), and sodium acetate (2.2 g) was added. A solution of bromine (0.70 mL) in acetic acid (13 mL) was added slowly. The reaction was stirred at room temperature overnight. The reaction was carefully added to 2M aqueous Na₂CO₃ and extracted with ethyl acetate. The organic layer was washed with brine and dried over sodium sulfate. After filtration and concentration, purification by silica gel chromatography, eluting with heptanes/ethyl acetate (9/1), gave the title compound. MS (DCI) m/e 490.1, 492.1 (M+H)⁺.

### 1.5.5. 8-bromo-5-(2-(tert-butyldiphenylsilyl)ethoxy)-1,2,3,4-tetrahydroisoquinoline

Example 1.5.4 (4.46 g) was dissolved in methanol (45 mL). Sodium cyanoborohydride (2.0 g) was added followed by trifluoroborane etherate (4.0 mL, 31.6 mmol). The mixture was heated under reflux for two hours and then cooled to room temperature. Additional sodium cyanoborohydride (2.0 g) and trifluoroborane etherate (4.0 mL) were added, and the mixture was heated under reflux for two more hours. The reaction was cooled, then added to 1/1 water/2M aqueous Na₂CO₃ (150 mL). The mixture was extracted with dichloromethane (twice with 100 mL). The organic layer was dried over sodium sulfate. Filtration and concentration provided the title compound that was used in the next step with no further purification. MS (DCI) m/e 494.1. 496.1 (M+H)⁺.

### 1.5.6, tert-butyl 8-bromo-5-(2-(tert-butyldiphenylsilyl)ethoxy)-3,4-dihydroisoquinoline-2(1H)-carboxylate

Example 1.5.5 (3.9 g) was dissolved in dichloromethane (25 mL), and triethylamine (3.3 mL) and di-tert-butyl dicarbonate (1.9 g) were added. The reaction mixture was stirred at room temperature for three hours. The reaction was then concentrated and purified by flash chromatography, eluting with heptanes/ethyl acetate (96/4), to provide the title compound.

### 1.5.7. 2-tert-butyl 8-methyl 5-(2-(tert-butyldiphenylsilyl)ethoxy)-3,4-dihydroisoquinoline-2,8(1H)-dicarboxylate

Example 1.5.6 (3.6 g) and [1,1′-bis(diphenylphosphino)ferrocene]dichloropalladium(II) dichloromethane (0.025 g) were placed in a 250 mL SS pressure bottle, and methanol (10 mL) and triethylamine (0.469 mL) were added. After degassing the reactor with argon several times, the flask was charged with carbon monoxide and heated to 100° C. for 16 hours at 40 psi. The reaction mixture was cooled, concentrated, and purified by flash silica gel chromatography, eluting heptanes/ethyl acetate (88/12), to provide the title compound.

### 1.5.8, methyl 5-(2-(tert-butyldiphenylsilyl)ethoxy)-1,2,3,4-tetrahydroisoquinoline-8-carboxylate

Example 1.5.7 (1.8 g) was dissolved in 4N HCl in dioxane (25 mL) and stirred at room temperature for 45 minutes. The reaction was then concentrated to provide the title compound as a hydrochloride salt. MS (DCI) m/e 474.2 (M+H)⁺.

### 1.5.9, methyl 2-(5-bromo-6-(tert-butoxycarbonyl)pyridin-2-yl)-5-(2-(tert-butyldiphenylsilyl)ethoxy)-1,2,3,4-tetrahydroisoquinoline-8-carboxylate

To a solution of Example 1.5.8 (1.6 g) and Example 1.4.4 (1.0 g) in dimethyl sulfoxide (6 mL) was added N,N-diisopropylethylamine (1.4 mL). The mixture was stirred at 50° C. for 24 hours. The mixture was then diluted with diethyl ether and washed with water and brine, and dried over Na₂SO₄. Filtration and evaporation of the solvent and silica gel column purification (eluting with 5% ethyl acetate in hexane) gave the title compound.

### 1.5.10. 1-((3-(2-azidoethoxy)-5,7-dimethyladamantan-1-yl)methyl)-4-iodo-5-methyl-1H-pyrazole

Example 1.1.6 (2 g) was dissolved in dichloromethane (20 mL), and triethylamine (0.84 mL) was added. After cooling the reaction solution to 5° C., mesyl chloride (0.46 mL) was added dropwise. The cooling bath was removed and the reaction was stirred at room temperature for two hours. Saturated NaHCO₃ was added, the layers were separated, and the organic layer was washed with brine, and dried over Na₂SO₄. After filtration and concentration, the residue was dissolved in N,N dimethylformamide (15 mL) and sodium azide (0.88 g) was added, and the reaction was heated to 80° C. for two hours. The reaction was then cooled to room temperature and poured into diethyl ether and water. The organic layer was separated and washed with brine and dried over Na₂SO₄. After filtration and concentration, purification by silica gel chromatography, eluting with heptanes/ethyl acetate (4/1), gave the title compound. MS (DCI) m/e 470.0 (M+H)⁺.

### 1.5.11, methyl 2-(6-(tert-butoxycarbonyl)-5-(4,4,5,5-tetramethyl-1,3,2-dioxaborolan-2-yl)pyridin-2-yl)-5-(2-(tert-butyldiphenylsilyl)ethoxy)-1,2,3,4-tetrahydroisoquinoline-8-carboxylate

Example 1.5.9 (1.5 g), 4,4,5,5-tetramethyl-1,3,2-dioxaborolane (0.46 mL), [1,1′-bis(diphenylphosphino)ferrocene]dichloropalladium(II) dichloromethane (86 mg), and triethylamine (0.59 mL) were dissolved in acetonitrile (6.5 mL) under a nitrogen atmosphere, then the reaction was heated under reflux overnight. The reaction was then cooled to room temperature and ethyl acetate and water were added. The organic layer was washed with brine and dried over Na₂SO₄. After filtration and concentration, purification by silica gel chromatography, using a gradient of 10-20% ethyl acetate in heptanes, gave the title compound. MS (ESI) m/e 777.1 (M+H)⁺.

### 1.5.12, methyl 2-(5-(1-((3-(2-azidoethoxy)-5,7-dimethyladamantan-1-yl)methyl)-5-methyl-1H-pyrazol-4-yl)-6-(tert-butoxycarbonyl)pyridin-2-yl)-5-(2-(tert-butyldiphenylsilyl) ethoxy)-1,2,3,4-tetrahydroisoquinoline-8-carboxylate

Example 1.5.11 (1.22 g) and Example 1.5.10 (0.74 g) were dissolved in tetrahydrofuran (16 mL) under a nitrogen atmosphere, and tripotassium phosphate (4.5 g) and water (5 mL) were added. Tris(dibenzylideneacetone)dipalladium(0) (70 mg) and 1.3,5,7-tetramethyl-8-tetradecyl-2,4,6-trioxa-8-phosphaadamantane (66 mg) were then added, the reaction was heated at reflux overnight, and then allowed to cool to room temperature. Ethyl acetate and water were then added, and the organic layer washed with brine and dried over Na₂SO₄. After filtration and concentration, the crude material was purified by silica gel chromatography, eluting with heptanes/ethyl acetate (7/3), gave the title compound. MS (DCI) m/e 992.3 (M+H)⁺.

### 1.5.13. 2-(5-(1-((3-(2-azidoethoxy)-5,7-dimethyladamantan-1-yl)methyl)-5-methyl-1H-pyrazol-4-yl)-6-(tert-butoxycarbonyl)pyridin-2-yl)-5-(2-(tert-butyldiphenylsilyl)ethoxy)-1,2,3,4-tetrahydroisoquinoline-8-carboxylic acid

Example 1.5.12 (1.15 g) was dissolved in tetrahydrofuran (4.5 mL), and methanol (2.2 mL), water (2.2 mL), and lithium hydroxide monohydrate (96 mg) were added. The reaction mixture was stirred at room temperature for five days. Water (20 mL) and 2N aqueous HCl (1.1 mL) were added. The mixture was extracted with ethyl acetate, and the organic layer was washed with brine and dried over Na₂SO₄. After filtration and concentration, purification by silica gel chromatography, eluting with dichloromethane/ethyl acetate (70/30) followed by dichloromethane/ethyl acetate/acetic acid (70/30/1), gave the title compound.

### 1.5.14, tert-butyl 3-(1-((3-(2-azidoethoxy)-5,7-dimethyladamantan-1-yl)methyl)-5-methyl-1H-pyrazol-4-yl)-6-(8-(benzo[d]thiazol-2-ylcarbamoyl)-5-(2-(tert-butyldiphenylsilyl)ethoxy)-3,4-dihydroisoquinolin-2(1H)-yl)picolinate

Example 1.5.13 (80 mg) and benzo[d]thiazol-2-amine (14 mg) were dissolved in dichloromethane (1.2 mL). N,N-Dimethylpyridin-4-amine (17 mg) and N-ethyl-N′-(3-dimethylaminopropyl)carbodiimide hydrochloride (27 mg) were added and the reaction was stirred at room temperature overnight. The reaction was concentrated and the crude residue was purified by silica gel chromatography, eluting with dichloromethane/ethyl acetate (90/10), to provide the title compound. MS (ESI) m/e 1110.3 (M+H)⁺.

### 1.5.15, tert-butyl 3-(1-((3-(2-azidoethoxy)-5,7-dimethyladamantan-1-yl)methyl)-5-methyl-1H-pyrazol-4-yl)-6-(8-(benzo[d]thiazol-2-ylcarbamoyl)-5-hydroxy-3,4-dihydroisoquinolin-2(1H)-yl)picolinate

Example 1.5.14 (160 mg) was dissolved in a 1.0M solution of tetrabutylammonium fluoride in 95/5 tetrahydrofuran/water (1.15 mL) and the reaction was heated at 60° C. for two days. Powdered 4 Å molecular sieves were added, and the mixture was heated at 60° C. for another day. The reaction was cooled, then concentrated and the crude residue was purified by silica gel chromatography, eluting with 70/30/1 dichloromethane/ethyl acetate/acetic acid, to provide the title compound. MS (ESI) m/e 844.2 (M+H)⁺.

### 1.5.16, tert-butyl 3-(1-((3-(2-aminoethoxy)-5,7-dimethyladamantan-1-yl)methyl)-5-methyl-1H-pyrazol-4-yl)-6-(8-(benzo[d]thiazol-2-ylcarbamoyl)-5-hydroxy-3,4-dihydroisoquinolin-2(H)-yl)picolinate

Example 1.5.15 (70 mg) was dissolved in tetrahydrofuran (2 mL), 10% palladium on carbon (20 mg) was added, and the mixture was stirred under a hydrogen balloon overnight. After filtration through diatomaceous earth and evaporation of the solvent, the crude title compound was purified by reverse phase chromatography (C18 column), eluting with 10-90% acetonitrile in 0.1% TFA water, to provide the title compound as a trifluoroacetic acid salt.

### 1.5.17. 3-(1-((3-(2-aminoethoxy)-5,7-dimethyladamantan-1-yl)methyl)-5-methyl-1H-pyrazol-4-yl)-6-(8-(benzo[d]thiazol-2-ylcarbamoyl)-5-hydroxy-3,4-dihydroisoquinolin-2(l H)-yl)picolinic acid

Example 1.5.16 (11 mg) was dissolved in 4N HCl in dioxane (0.5 mL) and stirred at room temperature overnight. The solids were filtered off and washed with dioxane to provide the title compound as a hydrochloride salt. ¹H NMR (500 MHz, dimethyl sulfoxide-d₆) δ ppm 12.60 (v br s, 1H), 10.40 (br s, 1H), 8.00 (d, 1H) 7.76 (d, 1H), 7.75 (br s, 3H), 7.60 (d, 1H), 7.51 (d, 1H), 7.46 (t, 1H), 7.33 (t, 1H), 7.30 (s, 1H), 6.98 (d, 1H), 6.82 (d, 1H), 4.99 (s, 2H), 3.89 (m, 2H), 3.83 (s, 2H), 3.50 (m, 2H), 2.88 (m, 2H), 2.79 (m, 2H), 2.11 (s, 3H), 1.41 (s, 2H), 1.29 (m, 4H), 1.14 (m, 41H), 1.04 (m, 2H), 0.87 (s, 6H). MS (ESI) m/e 762.2 (M+H)⁺.

### 1.6. Synthesis of 6-[8-(1,3-benzothiazol-2-ylcarbamoyl)naphthalen-2-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid (Compound W3.06)

### 1.6.1, tert-butyl 3-(1-((3-(2-((tert-butoxycarbonyl)(methyl)amino) ethoxy)-5,7-dimethyladamantan-1-yl)methyl)-5-methyl-1H-pyrazol-4-yl)-6-(8-(methoxycarbonyl)naphthalen-2-yl)picolinate

To a solution of methyl 7-(4,4,5,5-tetramethyl-1,3,2-dioxaborolan-2-yl)-1-naphthoate (2.47 g) in dioxane (40 mL) and water (20 mL) was added Example 1.1.11 (4.2 g), bis(triphenylphosphine)palladium(II) dichloride (556 mg), and CsF (3.61 g). The mixture was stirred at reflux overnight. The mixture was diluted with ethyl acetate (400 mL) and washed with water and brine, and dried over Na₂SO₄. After filtration and evaporation of the solvent, the crude material was purified via column chromatography, eluting with 20% ethyl acetate in heptane followed by 5% methanol in dichloromethane, to provide the title compound. MS (ESI) m/e 793.4 (M+H)⁺.

### 1.6.2. 7-(6-(tert-butoxycarbonyl)-5-(1-((3-(2-((tert-butoxycarbonyl) (methyl)amino)ethoxy)-5,7-dimethyladamantan-1-yl)methyl)-5-methyl-1H-pyrazol-4-yl)pyridin-2-yl)-1-naphthoic acid

To a solution of Example 1.6.1 (500 mg) in tetrahydrofuran (4 mL), methanol (2 mL) and water (2 mL) was added lithium hydroxide monohydrate (500 mg). The mixture was stirred for 3 hours. The mixture was then acidified with 1N aqueous HCl and diluted with ethyl acetate (200 mL). The organic layer was washed with water and brine, and dried over Na₂SO₄. Filtration and evaporation of the solvent gave the crude title compound which was used in the next reaction without further purification. MS (ESI) m/e 779.4 (M+H)⁺.

### 1.6.3. 6-[8-(1,3-benzothiazol-2-ylcarbamoyl)naphthalen-2-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid

To a solution of Example 1.6.2 (79 mg) in N,N-dimethylformamide (2 mL) was added benzo[d]thiazol-2-amine (23 mg), fluoro-N,N,N′,N′-tetramethylformamidinium hexafluorophosphate (41 mg) and N,N-diisopropylethylamine (150 mg). The mixture was stirred at 60° C. for 3 hours. The reaction mixture was diluted with ethyl acetate (200 mL) and washed with water and brine, and dried over Na₂SO₄. Filtration and evaporation of the solvent gave a crude intermediate which was dissolved in dichloromethane/TFA (1:1, 6 mL) and left to sit overnight. Evaporation of the solvent gave a residue which was dissolved in dimethyl sulfoxide/methanol (1:1, 9 mL) and purified by HPLC (Gilson system, eluting with 10-85% acetonitrile in 0.1% TFA in water) to give the pure title compound. ¹H NMR (501 MHz, dimethyl sulfoxide-d₆) δ ppm 13.11 (s, 1H), 9.02 (s, 1H), 8.38 (dd, 1H), 8.26-8.34 (m, 2H), 8.13-8.27 (m, 3H), 8.07 (d, 1H), 8.02 (d, 1H), 7.93 (d, 1H), 7.82 (d, 1H), 7.67-7.75 (m, 1H), 7.44-7.53 (m, 2H), 7.30-7.41 (m, 1H), 3.90 (s, 3H), 2.94-3.12 (m, 3H), 2.53-2.60 (m, 4H), 2.20-2.31 (m, 3H), 1.45 (s, 2H), 1.25-1.39 (m, 4H), 0.99-1.23 (m, 4H), 0.89 (s, 6H). MS (ESI) m/e 755.4 (M+H)⁺.

### 1.7. Synthesis of 3-[1-({3,5-dimethyl-7-[2-(methylamino)ethoxy]tricyclo[3.3.1.1^{3,7}]dec-1-yl}methyl)-5-methyl-1H-pyrazol-4-yl]-6-[8-([1,3]thiazolo[5,4-b]pyridin-2-ylcarbamoyl)naphthalen-2-yl]pyridine-2-carboxylic acid (Compound W3.07)

The title compound was prepared by substituting thiazolo[5,4-b]pyridin-2-amine for benzo[d]thiazol-2-amine in Example 1.6.3. ¹H NMR (400 MHz, dimethyl sulfoxide-d₆) δ ppm 13.25 (s, 1H), 9.02 (s, 1H), 8.54 (dd, 1H), 8.39 (dd, 1H), 8.14-8.35 (m, 6H), 8.04 (d, 1H), 7.93 (d, 1H), 7.66-7.75 (m, 1H), 7.55 (dd, 1H), 7.49 (s, 1H), 3.57 (t, 3H), 2.95-3.10 (m, 2H), 2.51-2.62 (m, 3H), 2.19-2.28 (m, 3H), 1.45 (s, 2H), 1.24-1.38 (m, 4H), 0.98-1.24 (m, 6H), 0.89 (s, 6H). MS (ESI) m/e 756.3 (M+H)⁺.

### 1.8. Synthesis of 3-[1-((3,5-dimethyl-7-[2-(methylamino)ethoxy]tricyclo[3.3.1.1^{3,7}]dec-1-yl)methyl)-5-methyl-1H-pyrazol-4-yl]-6-[8-([1,3]thiazolo[4,5-b]pyridin-2-ylcarbamoyl)naphthalen-2-yl]pyridine-2-carboxylic acid (Compound W3.08)

The title compound was prepared by substituting thiazolo[4,5-c]pyridin-2-amine for benzo[d]thiazol-2-amine in Example 1.6.3. ¹H NMR (501 MHz, dimethyl sulfoxide-d₆) δ ppm 13.40 (s, 1H), 9.04 (s, 1H), 8.62 (dd, 1H), 8.56 (dd, 1H), 8.39 (dd, 1H), 8.13-8.34 (m, 5H), 8.06 (d, 1H), 7.94 (d, 1H), 7.68-7.79 (m, 1H), 7.45-7.54 (m, 1H), 7.39 (dd, 1H), 3.90 (s, 3H), 3.54-3.60 (m, 3H), 2.94-3.08 (m, 2H), 2.51-2.60 (m, 4H), 2.18-2.31 (m, 3H), 1.46 (s, 2H), 1.24-1.40 (m, 4H), 1.01-1.21 (m, 6H), 0.83-0.89 (m, 5H). MS (ESI) m/e 756.3 (M+H)⁺.

### 1.9. Synthesis of 6-[8-(1,3-benzothiazol-2-ylcarbamoyl)-5-methoxy-3,4-dihydroisoquinolin-2(1H)-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid (Compound W3.09)

### 1.9.1, tert-butyl 8-bromo-5-hydroxy-3,4-dihydroisoquinoline-2(1H)-carboxylate

To a solution of tert-butyl 5-hydroxy-3,4-dihydroisoquinoline-2(1H)-carboxylate (9 g) in N,N-dimethylformamide (150 mL) was added N-bromosuccinimide (6.43 g). The mixture was stirred overnight and quenched with water (200 mL). The mixture was diluted with ethyl acetate (500 mL) and washed with water and brine, and dried over sodium sulfate. Filtration and evaporation of the solvent gave crude title compound which was used in the next reaction without further purification. MS(ESI) m/e 329.2 (M+H)⁺.

### 1.9.2, tert-butyl 5-(benzyloxy)-8-bromo-3,4-dihydroisoquinoline-2(1H)-carboxylate

To a solution of Example 1.9.1 (11.8 g) in acetone (200 mL) was added benzyl bromide (7.42 g) and K₂CO₃ (5 g). The mixture was stirred at reflux overnight. The mixture was concentrated and the residue was partitioned between ethyl acetate (600 mL) and water (200 mL). The organic layer was washed with water and brine, and dried over sodium sulfate. Filtration and evaporation of the solvent gave crude title compound which was purified on a silica gel column and eluted with 10% ethyl acetate in heptane to provide the title compound. MS (ESI) m/e 418.1 (M+H)⁺.

### 1.93. 2-tert-butyl 8-methyl 5-(benzyloxy)-3,4-dihydroisoquinoline-2,8(1H)-dicarboxylate

Methanol (100 mL) and triethylamine (9.15 mL) were added to Example 1.9.2 (10.8 g) and [1,1′-bis(diphenylphosphino)ferrocene]dichloropalladium(II) (0.48 g) in a 500 mL stainless steel pressure reactor. The vessel was sparged with argon several times. The reactor was pressurized with carbon monoxide and stirred for 2 hours at 100° C. under 60 psi of carbon monoxide. After cooling, the crude reaction mixture was concentrated under vacuum. The residue was partitioned between ethyl acetate (500 mL) and water (200 mL). The organic layer was further washed with water and brine, and dried over sodium sulfate. After filtration and evaporation of the solvent, the residue was purified on a 330 g silica gel column, eluting with 10-20% ethyl acetate in heptane, to provide the title compound. MS (ESI) m/e 398.1 (M+H)⁺.

### 1.9.4, methyl 5-(benzyloxy)-1,2,3,4-tetrahydroisoquinoline-8-carboxylate hydrochloride

To a solution of Example 1.9.3 (3.78 g) in tetrahydrofuran (20 mL) was added 4N HCl in dioxane (20 mL). The mixture was stirred overnight and the mixture was concentrated under vacuum and the crude title compound was used in the next reaction without further purification. MS (ESI) me 298.1 (M+H)⁺.

### 1.9.5. methyl 5-(benzyloxy)-2-(5-bromo-6-(tert-butoxycarbonyl)pyridin-2-yl)-1,2,3,4-tetrahydroisoquinoline-8-carboxylate

To a solution of Example 1.9.4 (3.03 g) in dimethyl sulfoxide (50 mL) was added Example 1.4.4 (2.52 g) and triethylamine (3.8 mL). The mixture was stirred at 60° C. overnight under nitrogen. The reaction mixture was diluted with ethyl acetate (500 mL) and washed with water and brine, and dried over sodium sulfate. After filtration and evaporation of the solvent, the crude material was purified on a silica gel column, eluting with 20% ethyl acetate in heptane, to give the title compound. MS (ESI) m/e 553.1 (M+H)⁺.

### 1.9.6, methyl 5-(benzyloxy)-2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-1,2,3,4-tetrahydroisoquinoline-8-carboxylate

To a solution of Example 1.9.5 (2.58 g) in tetrahydrofuran (40 mL) and water (20 mL) was added Example 1.1.10 (2.66 g), 1,3,5,7-tetramethyl-6-phenyl-2,4,8-trioxa-6-phosphaadamante (341 mg), tris(dibenzylideneacetone)dipalladium(0) (214 mg), and K₃PO₄ (4.95 g). The mixture was stirred at reflux for 4 hours. The mixture was diluted with ethyl acetate (500 mL) and washed with water and brine, and dried over sodium sulfate. After filtration and evaporation of the solvent, the crude material was purified on a silica gel column, eluting with 20% ethyl acetate in dichloromethane, to give the title compound. MS (ESI) m/e 904.5 (M+H)⁺.

### 1.9.7, methyl 2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-5-hydroxy-1,2,3,4-tetrahydroisoquinoline-8-carboxylate

Example 1.9.6 (3.0 g) in tetrahydrofuran (60 mL) was added to Pd(OH)₂ (0.6 g, Degussa #E101NE/W, 20% on carbon, 49% water content) in a 250 mL SS pressure bottle. The mixture was agitated for 16 hours under 30 psi of hydrogen gas at 50° C. The mixture was then filtered through a nylon membrane, and the solvent concentrated under vacuum to provide the title compound. MS (ESI) m/e 815.1(M+H)⁺.

### 1.9.8, methyl 2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-5-methoxy-1,2,3,4-tetrahydroisoquinoline-8-carboxylate

Example 1.9.7 (170 mg) was dissolved in dichloromethane (0.8 mL) and methanol (0.2 mL). To the mixture was added a 2.0M solution of (trimethylsilyl)diazomethane in diethyl ether (0.17 mL) and the reaction was stirred at room temperature overnight. Additional 2.0M (trimethylsilyl)diazomethane in diethyl ether (0.10 mL) was added, and the reaction was allowed to stir for 24 hours. The reaction mixture was then concentrated and the title compound was used without further purification. MS (ESI) m/e 828.2 (M+H)⁺.

### 1.9.9. 2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-5-methoxy-1,2,3,4-tetrahydroisoquinoline-8-carboxylic acid

The title compound was prepared by substituting Example 1.9.8 for Example 1.5.12 in Example 1.5.13. MS (ESI) m/e 814.1 (M+H)⁺.

### 1.9.10, tert-butyl 6-(8-(benzo[d]thiazol-2-ylcarbamoyl)-5-methoxy-3,4-dihydroisoquinolin-2(1H)-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

The title compound was prepared by substituting Example 1.9.9 for Example 1.5.13 in Example 1.5.14. MS (ESI) m/e 946.1 (M+H)⁺.

### 1.9.11. 6-(8-(benzo[d]thiazol-2-ylcarbamoyl)-5-methoxy-3,4-dihydroisoquinolin-2(1H)-yl)-3-(1-((3,5-dimethyl-7-(2-(methylamino)ethoxy)adamantan-1-yl)methyl)-5-methyl-1H-pyrazol-4-yl)picolinic acid

The title compound was prepared by substituting Example 1.9.10 for Example 1.5. 16 in Example 1.5.17. ¹H NMR (500 MHz, dimethyl sulfoxide-d₆) δ ppm 8.74 (br s, 2H), 8.02 (d, 1H) 7.77 (m, 2H), 7.54 (d, 1H), 7.47 (t, 1H), 7.34 (m, 2H), 7.01 (d, 2H), 5.01 (s, 2H), 3.90 (m, 2H), 3.89 (s, 3H), 3.85 (s, 21H), 3.58 (m, 2H), 3.57 (s, 3H), 2.98 (m, 2H), 2.82 (m, 21H), 2.12 (s, 3H), 1.41 (s, 2H), 1.30 (m, 4H), 1.14 (m, 4H), 1.04 (m, 2H), 0.87 (s, 6H). MS (ESI) m/e 790.2 (M+H)⁺.

### 1.10. Synthesis of 6-[5-(1,3-benzothiazol-2-ylcarbamoyl)quinolin-3-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid (Compound W3.10)

### 1.10.1. 3-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)quinoline-5-carboxylic acid

A mixture of 3-bromoquinoline-5-carboxylic acid (300 mg), 4,4,4′,4′,5,5,5′,5′-octamethyl-2,2′-bi(1,3,2-dioxaborolane) (363 mg), and potassium acetate (350 mg) in dioxane (5 mL) was purged with nitrogen gas for 5 minutes, and PdCl₂(dppf)-CH₂Cl₂ adduct (58.3 mg) was added. The mixture was heated at 100° C. overnight and cooled. To this mixture was added Example 1.1.11 (510 mg), dichlorobis(triphenylphosphine)-palladium(II) (83 mg), CsF (362 mg), and water (3 mL). The resulting mixture was heated at 100° C. overnight and filtered through diatomaceous earth. The filtrate was concentrated, and the residue was dissolved in dimethyl sulfoxide, loaded onto a C18 column (300 g), and eluted with a gradient of 50-100% acetonitrile in a 0.1% TFA/water solution to provide the title compound. MS (ESI) m/e 780.5 (M+H)⁺.

### 1.10.2, tert-butyl 6-(5-(benzo[d]thiazol-2-ylcarbamoyl)quinolin-3-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

To a mixture of Example 1.10.1 (120 mg), benzo[d]thiazol-2-amine (46.2 mg), and O-(7-azabenzotriazol-1-yl)-N,N,N′,N′-tetramethyluronium hexafluorophosphate (HATU, 117 mg) in N,N-dimethylformamide (0.5 mL) was added N,N-diisopropvlethylamine (134 μL). The mixture was stirred overnight and loaded onto a C18 column (300 g), eluting with a gradient of 50-100% acetonitrile in 0.1% TFA/water solution to provide the title compound. MS (ESI) m/e 913.4 (M+H)⁺.

### 1.10.3. 6-[5-(1,3-benzothiazol-2-ylcarbamoyl)quinolin-3-yl]-3-[1-((3,5-dimethyl-7-[2-(methylamino)ethoxy]tricyclo[3.3.1.1^{3,7}]dec-1-yl)methyl)-5-methyl-1H-pyrazol-4-yl]pyridine-2-carboxylic acid

Example 1.10.2 (50 mg) in dichloromethane (3 mL) was treated with trifluoroacetic acid (2 mL) overnight and concentrated. The residue was dissolved in a mixture of dimethyl sulfoxide (5 mL), loaded onto a C18 column (300 g), and eluted with a gradient of 10-70% acetonitrile in 0. 1% TFA water solution to provide the title compound. ¹H NMR (400 MHz, dimethyl sulfoxide-d₆) δ ppm 13.22 (s, 1H), 9.73 (d, 1H), 9.41 (s, 1H), 8.34 (dd, 2H), 8.27 (s, 3H), 8.18 (d, 1H), 8.08 (d, 1H), 8.02-7.93 (m, 2H), 7.82 (d, 1H), 7.55-7.46 (m, 2H), 7.38 (t, 1H), 3.91 (s, 2H), 3.03 (p, 2H), 2.59-2.53 (m, 4H), 2.25 (s, 3H), 1.46 (s, 2H), 1.38-1.25 (m, 4H), 1.18 (s, 4H), 1.11-1.01 (m, 2H), 0.89 (s, 6H). MS (ESI) m/e 756.2 (M+H)⁺.

### 1.11. Synthesis of 6-[4-(1,3-benzothiazol-2-ylcarbamoyl)quinolin-6-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid (Compound W3.11)

### 1.11.1, ethyl 6-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)quinoline-4-carboxylate

The title compound was prepared as described in Example 1.10.1, replacing 3-bromoquinoline-5-carboxylic acid with ethyl 6-bromoquinoline-4-carboxylate. MS (ESI) m/e 808.4 (M+H)⁺.

### 1.11.2. 6-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)quinoline-4-carboxylic acid

To a solution of Example 1.11.1 (100 mg) in dimethyl sulfoxide (2 mL) was added methanol (2 mL) and 1M lithium hydroxide (248 μL). The mixture was stirred for 30 minutes, acidified to pH 4 with 10% HCl, diluted with ethyl acetate and washed with water and brine to provide the title compound. MS (ESI) m/e 780.4 (M+H)⁺.

### 1.11.3, tert-butyl 6-(4-(benzo[d]thiazol-2-ylcarbamoyl)quinolin-6-yl)-3-(1-((3-(2-((tert-butoxycarbonyl)(amino)ethoxy)-5,7-dimethyladamantan-1-yl)methyl)-5-methyl-1H-pyrazol-4-yl)picolinate

The title compound was prepared as described in Example 1.10.2, replacing Example 1.10.1 with Example 1.11.2. MS (ESI) m/e 912.3 (M+H)⁺.

### 1.11.4. 6-[4-(1,3-benzothiazol-2-ylcarbamoyl)quinolin-6-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic acid

The title compound was prepared as described in Example 1.10.3, replacing Example 1.10.2 with Example 1.11.3. ¹H NMR (400 MHz, dimethyl sulfoxide-d₆) δ ppm 13.34 (s, 2H), 9.14 (d, 1H), 8.94 (s, 1H), 8.63 (dd, 1H), 8.27 (dd, 4H), 8.09 (d, 1H), 8.00-7.90 (m, 2H), 7.83 (d, 1H), 7.50 (d, 2H), 7.40 (t, 1H), 3.90 (s, 2H), 3.03 (p, 2H), 2.56 (t, 4H), 2.23 (s, 3H), 1.45 (s, 2H), 1.32 (d, 3H), 1.18 (s, 4H), 1.11-0.98 (m, 2H), 0.89 (s, 6H). MS (ESI) m/e 756.2 (M+H)⁺.

### 1.12. Synthesis of 6-[8-(1,3-benzothiazol-2-ylcarbamoyl)-5-methoxy-3,4-dihydroisoquinolin-2(1H)-yl]-3-{1-[(3-{2-[(2-methoxyethyl)amino]ethoxy}-5,7-dimethyltricyclo[3.3.1.1^{3,7}]dec-1-yl)methyl]-5-methyl-1H-pyrazol-4-yl}pyridine-2-carboxylic acid (Compound W3.12)

### 1.12.1, methyl 5-(benzyloxy)-2-(6-(tert-butoxycarbonyl)-5-(4,4,5,5-tetramethyl-1,3,2-dioxaborolan-2-yl)pyridin-2-yl)-1,2,3,4-tetrahydroisoquinoline-8-carboxylate

The title compound was prepared by substituting Example 1.9.5 for Example 1.5.9 in Example 1.5.11. MS (DCT) m/e 601.0 (M+H)⁺.

### 1.12.2. 2-((3-((4-iodo-5-methyl-1H-pyrazol-1-yl)methyl)-5,7-dimethyladamantan-1-yl)oxy)acetaldehyde

Dimethyl sulfoxide (4.8 mL) was dissolved in dichloromethane (150 mL). The mixture was cooled to −75° C., and oxalyl chloride (2.6 mL) was added dropwise. The reaction mixture was stirred at −75° C. for 45 minutes, and a solution of Example 1.1.6 (7.1 g) in dichloromethane (45 mL) was added dropwise. The reaction mixture was stirred at −75° C. for 30 minutes, and triethylamine (5.0 mL) was added. The reaction was warmed to room temperature, poured into water, and extracted with diethyl ether. The organic layer was washed with brine and dried over Na₂SO₄. After filtration and concentration, purification by silica gel chromatography, eluting with dichloromethane/ethyl acetate 85/15, gave the title compound. MS (DCI) m/e 443.0 (M+H)⁺.

### 1.12.3. 2-((3-((4-iodo-5-methyl-1H-pyrazol-1-yl)methyl)-5,7-dimethyladamantan-1-yl)oxy)-N-(2-methoxyethyl)ethanamine

Example 1.12.2 (4.0 g) and 2-methoxyethanamine (0.90 mL) were dissolved in dichloromethane (40 mL) and the mixture was stirred at room temperature for two hours. A suspension of sodium borohydride (500 mg) in methanol (7 mL) was added and the resulting mixture was stirred for 45 minutes. The reaction was then added to saturated aqueous NaHCO₃ and resultant mixture extracted with ethyl acetate. The organic layer was washed with brine and dried over Na₂SO₄. The title compound was obtained after filtration and concentration and was used without purification. MS (DCI) m/e 502.1 (M+H)⁺.

### 1.12.4, tert-butyl (2-((3-((4-iodo-5-methyl-1H-pyrazol-1-yl)methyl)-5,7-dimethyladamantan-1-yl)oxy)ethyl)(2-methoxyethyl)carbamate

Example 1.12.3 (4.4 g) was dissolved in tetrahydrofuran (60 mL), and di-tert-butyl dicarbonate (3.0 g) and N,N-dimethylpyridin-4-amine (0.15 g) were added. The reaction was stirred at room temperature overnight. The reaction was then concentrated and purified by flash chromatography, eluting with dichloromethane/ethyl acetate (3/1), to provide the title compound.

### 1.12.5, methyl 5-(benzyloxy)-2-(6-(tert-butoxycarbonyl)-5-(1-((3-(2-((tert-butoxycarbonyl)(2-methoxyethyl)amino)ethoxy)-5,7-dimethyladamantan-1-yl)methyl)-5-methyl-1H-pyrazol-4-yl)pyridin-2-yl)-1,2,3,4-tetrahydroisoquinoline-8-carboxylate

The title compound was prepared by substituting Example 1.12.1 for Example 1.5.11 and Example 1.12.4 for Example 1.5.10 in Example 1.5.12. MS (ESI) m/e 948.2 (M+H)⁺.

### 1.12.6, methyl 2-(6-(tert-butoxycarbonyl)-5-(1-((3-(2-((tert-butoxycarbonyl)(2-methoxyethyl)amino)ethoxy)-5,7-dimethyladamantan-1-yl)methyl)-5-methyl-1H-pyrazol-4-yl)pyridin-2-yl)-5-hydroxy-1,2,3,4-tetrahydroisoquinoline-8-carboxylate

Example 1.12.5 (5.2 g) was dissolved in tetrahydrofuran (100 mL). 20% Palladium hydroxide on activated charcoal (1.0 g) was then added, and the reaction mixture agitated on a Parr rector under a hydrogen atmosphere at 30 psi and 50° C. for 3 hours. After filtration and concentration, purification by silica gel chromatography, eluting with heptanes/ethyl acetate (2/3), gave the title compound. MS (ESI) m/e 858.1 (M+H)⁺.

### 1.12.7, methyl 2-(6-(tert-butoxycarbonyl)-5-(l-((3-(2-((tert-butoxycarbonyl)(2-methoxyethyl)amino)ethoxy)-5,7-dimethyladamantan-1-yl)methyl)-5-methyl-1H-pyrazol-4-yl)pyridin-2-yl)-5-methoxy-1,2,3,4-tetrahydroisoquinoline-8-carboxylate

The title compound was prepared by substituting Example 1.12.6 for Example 1.9.7 in Example 1.9.8. MS (ESI) m/e 872.2 (M+H)⁺.

### 1.12.8. 2-(6-(tert-butoxycarbonyl)-5-(1-((3-(2-((tert-butoxycarbonyl)(2-methoxyethyl)amino)ethoxy)-5,7-dimethyladamantan-1-yl)methyl)-5-methyl-1H-pyrazol-4-yl)pyridin-2-yl)-5-methoxy-1,2,34-tetrahydroisoquinoline-8-carboxylic acid

The title compound was prepared by substituting Example 1.12.7 for Example 1.5.12 in Example 1.5.13. MS (ESI) m/e 858.1 (M+H)⁺.

### 1.12.9, tert-butyl 6-(8-(benzo[d]thiazol-2-ylcarbamoyl)-5-methoxy-3,4-dihydroisoquinolin-2(1H)-yl)-3-(1-((3-(2-((tert-butoxycarbonyl)(2-methoxyethyl)amino)ethoxy)-5,7-dimethyladamantan-1-yl)methyl)-5-methyl-1H-pyrazol-4-yl)picolinate

The title compound was prepared by substituting Example 1.12.8 for Example 1.5.13 in Example 1.5.14. MS (ESI) m/e 990.1 (M+H)⁺.

### 1.12.10. 6-(8-(benzo[d]thiazol-2-ylcarbamoyl)-5-methoxy-3,4-dihydrisoquinolin-2(1H)-yl)-3-(1-(((1r,3s,5R,7S)-3-(2-((2-methoxyethyl)amino)ethoxy)-5,7-dimethyladamantan-1-yl)methyl)-5-methyl-1H-pyrazol-4-yl)picolinic acid

Example 1.12.9 (2.6 g) was dissolved in dioxane (20 mL), then 4N HCl in dioxane (100 mL) was added, and the reaction was stirred at room temperature overnight. The precipitants were allowed to settle and the supernatant was drawn off. The remaining solids were purified by reverse phase chromatography (C18 column), eluting with 10-90% acetonitrile in 0.1% TFA/water, to provide the title compound as a trifluoroacetic acid salt. ¹H NMR (500 MHz, dimethyl sulfoxide-d₆) δ ppm 8.41 (v br s, 2H), 8.01 (d, 1H) 7.77 (m, 2H), 7.50 (d, 1H), 7.47 (m, 1H), 7.34 (t, 1H), 7.29 (s, 1H), 7.01 (dd, 2H), 5.00 (s, 2H), 3.90 (m, 2H), 3.89 (s, 3H), 3.83 (s, 2H), 3.56 (m, 4H), 3.29 (s, 3H), 3.12 (m, 2H), 3.05 (m, 2H), 2.81 (m, 2H), 2.11 (s, 3H), 1.41 (s, 2H), 1.30 (m, 4H), 1.14 (m, 4H), 1.04 (m, 2H), 0.87 (s, 6H). MS (ESI) m/e 834.3 (M+H)⁺.

### 1.13. Synthesis of 3-(1-{[3-(2-aminoethoxy)-5,7-dimethyltricyclo[3.3.1.1′⁷]dec-1-yl]methyl}-5-methyl-1H-pyrazol-4-yl)-6-[8-(1,3-benzothiazol-2-ylcarbamoyl)-5-cyano-3,4-dihydroisoquinolin-2(1H)-yl]pyridine-2-carboxylic acid (Compound W3.13)

### 1.13.1. 4-Bromo-3-cyanomethyl-benzoic acid methyl ester

Trimethylsilanecarbonitrile (3.59 mL) was added to tetrahydrofuran (6 mL). 1M Tetrabutylammonium fluoride (26.8 mL) was added dropwise over 30 minutes. The solution was then stirred at room temperature for 30 minutes. Methyl 4-bromo-3-(bromomethyl)benzoate (7.50 g) was dissolved in acetonitrile (30 mL) and the resultant solution added to the first solution dropwise over 30 minutes. The solution was then heated to 80° C. for 30 minutes and then allowed to cool to room temperature. The solution was concentrated under reduced pressure and purified by flash column chromatography on silica gel, eluting with 20-30% ethyl acetate in heptanes. The solvent was evaporated under reduced pressure to provide the title compound.

### 1.13.2. 3-(2-Aminoethyl)-4-bromobenzoic acid methyl ester

Example 1.13.1 (5.69 g) was dissolved in tetrahydrofuran (135 mL), and 1 M borane (in tetrahydrofuran, 24.6 mL) was added. The solution was stirred at room temperature for 16 hours and then slowly quenched with methanol and 1M HCL. 4M HCl (150 mL) was added, and the solution was stirred at room temperature for 16 hours. The mixture was concentrated was reduced under reduced pressure, and the pH adjusted to between 11 and 12 using solid potassium carbonate. The solution was then extracted with dichloromethane (3×1(00) mL). The organic extracts were combined and dried over anhydrous sodium sulfate. The solution was filtered and concentrated under reduced pressure, and the material was purified by flash column chromatography on silica gel, eluting with 10-20% methanol in dichloromethane. The solvent was evaporated under reduced pressure to provide the title compound. MS (ESI) m/e 258, 260 (M+H)⁺.

### 1.13.3. 4-Bromo-3-[2-(2,2,2-trifluoroacetylamino)-ethyl]-benzoic acid methyl ester

Example 1.13.2 (3.21 g) was dissolved in dichloromethane (60 mL). The solution was cooled to 0° C., and triethylamine (2.1 mL) was added. Trifluoroacetic anhydride (2.6 mL) was then added dropwise. The solution was stirred at 0° C. for ten minutes and then allowed to warm to room temperature while stirring for one hour. Water (50 mL) was added and the solution was diluted with ethyl acetate (100 mL). 1M HCl was added (50 mL) and the organic layer was separated, washed with 1M HCl, and then washed with brine. The organic layer was then dried on anhydrous sodium sulfate. After filtration, the solvent was evaporated under reduced pressure to provide the title compound. MS (ESI) m/e 371, 373 (M+H)⁺.

### 1.13.4. 5-Bromo-2-(2,2,2-trifluoroacetyl)-1,2,3,4-tetrahydroisoquinoline-8-carboxylic acid methyl ester

Example 1.13.3 (4.40 g) and paraformaldehyde (1.865 g) were placed in a flask and concentrated sulfuric acid (32 mL) was added. The solution was stirred at room temperature for one hour. Cold water (120 mL) was added. The solution was extracted with ethyl acetate (3×100 mL). The extracts were combined, washed with saturated aqueous sodium bicarbonate (100 mL), washed with water (100 mL), and dried over anhydrous sodium sulfate. The solution was concentrated under reduced pressure, and the material was purified by flash column chromatography on silica gel, eluting with 20-30% ethyl acetate in heptanes. The solvent was evaporated under reduced pressure to provide the title compound. MS (ESI) m/e 366, 368 (M+H)⁺.

### 1.13.5. 5-Cyano-2-(2,2,2-trifluoroacetyl)-1,2,3,4-tetrahydroisoquinoline-8-carboxylic acid methyl ester

Example 1.13.4 (5(00) mg) and dicyanozinc (88 mg) were added to N,N-dimethylformamide (4 mL). The solution was degassed and flushed with nitrogen three times. Tetrakis(triphenylphosphine)palladium(0) (79 mg) was added, and the solution was degassed and flushed with nitrogen once. The solution was then stirred at 80° C. for 16 hours. The solution was cooled, diluted with 50% ethyl acetate in heptanes (20 mL), and washed with 1 M hydrochloric acid (15 mL) twice. The organic layer was washed with brine and dried over anhydrous sodium sulfate. The solution was filtered and concentrated under reduced pressure, and the material was purified by flash column chromatography on silica gel, eluting with 20-30% ethyl acetate in heptanes. The solvent was evaporated under reduced pressure to provide the title compound.

### 1.13.6. 5Cyano-1,2,3,4-tetrahydroisoquinoline-8-carboxylic acid methyl ester

Example 1.13.5 (2.00 g) was dissolved in methanol (18 mL) and tetrahydrofuran (18 mL). Water (9 mL) was added followed by potassium carbonate (1.064 g). The reaction was stirred at room temperature for 135 minutes and then diluted with ethyl acetate (100 mL). The solution was washed with saturated aqueous sodium bicarbonate and dried on anhydrous sodium sulfate. The solvent was filtered and evaporated under reduced pressure to provide the title compound. MS (ESI) m/e 217 (M+H)⁺.

### 1.13.7. 2-(5-Bromo-6-tert-butoxycarbonylpyridin-2-yl)-5-cyano-1,2,3,4-tetrahydroisoquinoline-8-carboxylic acid methyl ester

Example 1.13.6 (1.424 g) and Example 1.4.4 (1.827 g) were dissolved in dimethyl sulfoxide (13 mL). N,N-Diisopropylethylamine (1.73 mL) was added, and the solution was heated to 50° C. for 16 hours. Additional Example 1.4.4 (0.600 g) was added, and the solution was heated at 50° C. for another 16 hours. The solution was allowed to cool to room temperature, diluted with ethyl acetate (50 mL), washed with water (25 mL) twice, washed with brine, and then dried on anhydrous sodium sulfate. The solution was filtered and concentrated under reduced pressure, and the material was purified by flash column chromatography on silica gel, eluting with 20-50% ethyl acetate in heptanes. The solvent was evaporated under reduced pressure to provide the title compound. MS (ESI) m/e 472, 474 (M+H)⁺.

### 1.13.8. 2-[6-tert-Butoxycarbonyl-5-(4,4,5,5-tetramethyl-[1,3,2]dioxaborolan-2-yl)-pyridin-2-yl]-5-cyano-1,2,3,4-tetrahydroisoquinoline-8-carboxylic acid methyl ester

Example 1.13.7 (2.267 g) and triethylamine (1.34 mL) were added to acetonitrile (15 mL). The solution was degassed and flushed with nitrogen three times. 4.4,5,5-Tetramethyl-1,3,2-dioxaborolane (1.05 mL) was added followed by dichloro[1,1′-bis(diphenylphosphino)ferrocene]palladium(II) (196 mg). The solution was degassed and flushed with nitrogen once and heated to reflux for 16 hours. The solution was cooled, diluted with ethyl acetate (50 mL), washed with water (10 mL), washed with brine, and dried on anhydrous sodium sulfate. The solution was concentrated under reduced pressure, and the material was purified by flash column chromatography on silica gel, eluting with 20-30% ethyl acetate in heptanes. The solvent was evaporated under reduced pressure to provide the title compound. MS (ESI) m/e 520 (M+H)⁺.

### 1.13.9. 2-(6-tert-Butoxycarbonyl-5-(1-[5-(2-tert-butoxycarbonylamino-ethoxy)-3,7-dimethyl-adamantan-1-ylmethyl]-5-methyl-1H-pyrazol-4-yl)-pyridin-2-yl)-5-cyano-1,2,3,4-tetrahydro-isoquinoline-8-carboxylic acid methyl ester

Example 1.13.8 (140 mg) and Example 1.4.2 (146 mg) were dissolved in tetrahydrofuran (3 mL). Potassium phosphate (286 mg) and water (0.85 mL) were added. The solution was degassed and flushed with nitrogen three times. (1S,3R,5R 7S)-1,3,5,7-Tetramethyl-8-tetradecyl-2,4,6-trioxa-8-phosphaadamantane (11 mg) and tris(dibenzylideneacetone)dipalladium(0) (12 mg) were added, and the solution was degassed and flushed with nitrogen once. The solution was heated to 62° C. for 16 hours. The solution was cooled, then diluted with water (5 mL) and ethyl acetate (25 mL). The organic layer was separated and washed with brine and dried on anhydrous sodium sulfate. The solution was filtered and concentrated under reduced pressure, and the material was purified by flash column chromatography on silica gel, eluting with 30-50% ethyl acetate in heptanes. The solvent was evaporated under reduced pressure to provide the title compound. MS (ESI) m/e 809 (M+H)⁺.

### 1.13.10. 2-(6-tert-Butoxycarbonyl-5-(1-[5-(2-tert-butoxycarbonylamino-ethoxy)-3,7-dimethyl-adamantan-1-ylmethyl]-5-methyl-1H-pyrazol-4-yl)-pyridin-2-yl)-5-cyano-1,2,3,4-tetrahydro-isoquinoline-8-carboxylic acid

Example 1.13.9 (114 mg) was dissolved in tetrahydrofuran (0.7 mL) and methanol (0.35 mL). Water (0.35 mL) was added followed by lithium hydroxide monohydrate (11 mg). The solution was stirred at room temperature for 16 hours, and 1 M hydrochloric acid (0.27 mL) was added. Water (1 mL) was added and the solution was extracted with ethyl acetate (5 mL) three times. The extracts were combined and dried on anhydrous sodium sulfate and filtered. The solvent was evaporated under reduced pressure to provide the title compound. MS (ESI) m/e 795 (M+H)⁺.

### 1.13.11. 6-[8-(Benzothiazol-2-ylcarbamoyl)-5-cyano-3,4-dihydro-1H-isoquinolin-2-yl]-3-{1-[5-(2-tert-butoxycarbonylamino-ethoxy)-3,7-dimethyl-adamantan-1-ylmethyl]-5-methyl-1H-pyrazol-4-yl}-pyridine-2-carboxylic acid tert-butyl ester

Example 1.13.10 (89 mg) and benzo[d]thiazol-2-amine (18 mg) were dissolved in dichloromethane (1.2 mL). N-(3-Dimethylaminopropyl)-N′-ethylcarbodiimide hydrochloride (39 mg) and N,N-dimethylpyridin-4-amine (25 mg) were added, and the solution was stirred at room temperature for 16 hours. The material was purified by flash column chromatography on silica gel, eluting with 50% ethyl acetate in heptanes. The solvent was evaporated under reduced pressure to provide the title compound. MS (ESI) m/e 927 (M+H)⁺.

### 1.13.12. 3-(1-{([3-(2-aminoethoxy)-5,7-dimethyltricyclo[3.3.1^{3,7}]dec-1 yl]methyl}-5-methyl-1H-pyrazol-4-yl)-6-[8-(1,3-benzothiazol-2-ylcarbamoyl)-5-cyano-3,4-dihydroisoquinolin-2(1H)-yl]pyridine-2-carboxylic acid

Example 1.13.11 (44 mg) was dissolved in dichloromethane (1 mL). Trifluoroacetic acid (0.144 mL) was added and the solution stirred at room temperature for 16 hours. The solvents were then evaporated under reduced pressure, the residue was dissolved in dichloromethane (1 mL), and the solvent removed under reduced pressure. Diethyl ether was added (2 mL) and was removed under reduced pressure. Diethyl ether (2 mL) was added again and removed under reduced pressure to provide the title compound as the trifluoroacetic acid salt. ¹H NMR (400 MHz, dimethyl sulfoxide-d₆) δ ppm 8.52 (bs, 1H), 8.05 (d, 1H), 7.92 (d, 1H), 7.82-7.75 (m, 2H), 7.63 (m, 2H), 7.50 (dd, 2H), 7.42-7.28 (m, 3H), 7.16 (t, 1H), 7.04 (d, 1H), 4.98 (s, 2H), 3.96 (t, 2H), 3.83 (s, 2H), 3.49 (t, 2H), 3.15 (t, 2H), 2.90 (q, 2H), 2.10 (s, 3H), 1.41 (s, 2H), 1.35-1.22 (m, 4H), 1.18-0.99 (m, 6H), 0.87 (bs, 6H). MS (ESI) m/e 771 (M+H)⁺.

### 1.14. Synthesis of 6-[1-(1,3-benzothiazol-2-ylcarbamoyl)-1,2,3,4-tetrahydroquinolin-7-yl]-3-{1-[(3-{2-[(2-methoxyethyl)amino]ethoxy}-5,7-dimethyltricyclo[3.3.1.1^{3,7}]dec-1-yl)methyl]-5-methyl-1H-pyrazol-4-yl}pyridine-2-carboxylic acid (Compound W3.14)

### 1.14.1. 2-((3,5-dimethyl-7-((5-methyl-4-(4,4,5,5-tetramethyl-1,3,2-dioxaborolan-2-yl)-1H-pyrazol-1-yl)methyl)adamantan-1-yl)oxy)ethanol

To a solution of Example 1.1.6 (4.45 g) and PdCl₂(dppf)-CH₂Cl₂ adduct (409 mg) in acetonitrile (60 mL) was added triethylamine (5 mL) and pinacolborane (6.4 mL). The mixture was refluxed overnight. The mixture was used directly in the next step without work up. MS (ESI) m/e 444.80 (M+H)⁺.

### 1.14.2, tert-butyl 6-chloro-3-(1-((3-(2-hydroxyethoxy)-5,7-dimethyladamantan-1-yl)methyl)-5-methyl-1H-pyrazol-4-yl)picolinate

To a solution of tert-butyl 3-bromo-6-chloropicolinate (3.06 g) in tetrahydrofuran (50 mL) and water (20 mL) was added Example 1.14.1 (4.45 g), 1,3,5,7-tetramethyl-8-tetradecyl-2,4,6-trioxa-8-phosphaadamantane (0.732 g), Pd₂(dba)₃ (0.479 g), and K₃PO₄ (11 g). The mixture was stirred at reflux overnight and concentrated. The residue was dissolved in ethyl acetate (500 mL) and washed with water and brine. The organic layer was dried over Na₂SO₄, filtered, and concentrated. The residue was purified by flash chromatography, eluting with a gradient of 20-40% ethyl acetate in dichloromethane, to provide the title compound. MS (ESI) m/e 530.23 (M+H)⁺.

### 1.14.3, tert-butyl 6-chloro-3-(1-((3,5-dimethyl-7-(2-((methylsulfonyl)oxy)ethoxy)adamantan-1-yl)methyl)-5-methyl-1H-pyrazol-4-yl)picolinate

To a cooled (0° C.) stirring solution of Example 1.14.2 (3.88 g) in dichloromethane (30 mL) and triethylamine (6 mL) was added methanesulfonyl chloride (2.52 g). The mixture was stirred at room temperature for 4 hours, diluted with ethyl acetate (400 mL), and washed with water and brine. The organic layer was dried over Na₂SO₄. Filtration and evaporation of the solvents afforded the title compound. MS (ESI) m/e 608.20 (M+H)⁺.

### 1.14.4, tert-butyl 3-(1-((3-(2-aminoethoxy)-5,7-dimethyladamantan-1-yl)methyl)-5-methyl-1H-pyrazol-4-yl)-6-chloropicolinate

A solution of Example 1.14.3 (2.2 g) in 7N ammonium in CH₃OH (20 mL) was heated at 100° C. under microwave conditions (Biotage Initiator) for 45 minutes and concentrated to dryness. The residue was dissolved in ethyl acetate and washed with water and brine. The organic layer was dried over Na₂SO₄, filtered, and concentrated to provide the title compound. MS (ESI) m/e 529.33 (M+H)⁺.

### 1.14.5, tert-butyl 6-chloro-3-(1-((3,5-dimethyl-7-(2-(2-(trimethylsilyl)ethylsulfonamido)ethoxy)adamantan-1-yl)methyl)-5-methyl-1H-pyrazol-4-yl)picolinate

To a cooled (0° C.) solution of Example 1.14.4 (3.0 g) in dichloromethane (30 mL) was added triethylamine (3 mL), followed by 2-(trimethylsilyl)ethanesulfonyl chloride (2.3 g). The mixture was stirred at room temperature for 3 hours and concentrated to dryness. The residue was dissolved in ethyl acetate (400 mL) and washed with aqueous NaHCO₃, water, and brine. The residue was dried over Na₂SO₄, filtered, concentrated, and purified by flash chromatography, eluting with 20% ethyl acetate in heptane, to provide the title compound. MS (ESI) m/e 693.04 (M+H)⁺.

### 1.14.6, tert-butyl 6-chloro-3-(1-((3-(2-(N-(2-methoxyethyl)-2-(trimethylsilyl)ethylsulfonamido)ethoxy)-5,7-dimethyladamantan-1-yl)methyl)-5-methyl-1H-pyrazol-4-yl)picolinate

To a solution of Example 1.14.5 (415 mg) in toluene (15 mL) was added 2-methoxyethanol (91 mg), followed by cyanomethylenetributylphospsphorane (289 mg). The mixture was stirred at 70° C. for 3 hours and concentrated to dryness. The residue was purified by flash chromatography, eluting with 20% ethyl acetate in heptane, to provide the title compound. MS (ESI) m/e 751.04 (M+H)⁺.

### 1.14.7, tert-butyl 3-(1-((3-(2-(N-(2-methoxyethyl)-2-(trimethylsilyl)ethylsulfonamido)ethoxy)-5,7-dimethyladamantan-1-yl)methyl)-5-methyl-1H-pyrazol-4-yl)-6-(1,2,3,4-tetrahydroquinolin-7-yl)picolinate

To a solution of 7-(4,4,5,5-tetramethyl-1,3,2-dioxaborolan-2-yl)-1,2,3,4-tetrahydroquinoline (172 mg) in dioxane (10 mL) and water (5 mL) was added Example 1.14.6 (500 mg), (Ph₃P)₂PdCl₂ (45.6 mg) and CsF (296 mg). The mixture was stirred at 120° C. for 30 minutes under microwave conditions (Biotage Initiator), diluted with ethyl acetate (200 mL) and washed with water and brine. The organic layer was dried over Na₂SO₄, filtered, and concentrated. The residue was purified by flash chromatography, eluting with 20% ethyl acetate in dichloromethane, to provide the title compound. MS (ESI) m/e 848.09 (M+H)⁺.

### 1.14.8, tert-butyl 6-(1-(benzo[d]thiazol-2-ylcarbamoyl)-1,2,3,4-tetrahydroquinolin-7-yl)-3-(1-((3-(2-(N-(2-methoxyethyl)-2-(trimethylsilyl)ethylsulfonamido)ethoxy)-5,7-dimethyladamantan-1-yl)methyl)-5-methyl-1H-pyrazol-4-yl)picolinate

To a suspension of bis(2,5-dioxopyrrolidin-1-yl) carbonate (63 mg) in acetonitrile (10 mL) was added benzo[d]thiazol-2-amine (37.2 mg). The mixture was stirred for 1 hour. A solution of Example 1.14.7 (210 mg) in acetonitrile (2 mL) was added, and the suspension was vigorously stirred overnight, diluted with ethyl acetate, and washed with water and brine. The organic layer was dried over Na₂SO₄, filtered, and concentrated to provide the title compound. MS (ESI) m/e 1024.50 (M+H)⁺.

### 1.14.9. 6-[1-(1,3-benzothiazol-2-ylcarbamoyl)-1,2,3,4-tetrahydroquinolin-7-yl]-3-{1-[(3-{2-[(2-methoxyethyl)amino]ethoxy}-5,7-dimethyltricyclo[3.3.1.1^{3,7}]dec-1-yl)methyl]-5-methyl-1H-pyrazol-4-yl}pyridine-2-carboxylic acid

To a solution of Example 1.14.8 (230 mg) in tetrahydrofuran (10 mL) was added tetrabutyl ammonium fluoride (TBAF 10 mL, 1M in tetrahydrofuran). The mixture was stirred at room temperature overnight, diluted with ethyl acetate, and washed with water and brine. The organic layer was dried over Na₂SO₄, filtered, and concentrated. The residue was dissolved in dichloromethane (5 mL) and treated with trifluoroacetic acid (5 mL) overnight. The mixture was concentrated, and the residue was purified by reverse HPLC (Gilson), eluting with 10-85% acetonitrile in 0.1% TFA/water to provide the title compound. ¹H NMR (400 MHz, dimethyl sulfoxide-d₆) δ ppm 8.40 (d, 3H), 8.00 (d, 1H), 7.90-7.72 (m, 3H), 7.46 (s, 1H), 7.40-7.32 (m, 1H), 7.28 (d, 1H), 7.24-7.17 (m, 1H), 3.95 (d, 3H), 3.88 (s, 16H), 3.56 (dt, 5H), 3.28 (s, 3H), 3.18-2.96 (m, 5H), 2.82 (t, 2H), 2.21 (s, 3H), 1.93 (p, 2H), 1.43 (s, 2H), 1.30 (q, 5H), 1.21-0.97 (m, 7H), 0.86 (s, 6H) MS (ESI) m/e 804.3 (M+H)⁺.

### 1.15. Synthesis of 6-[8-(1,3-benzothiazol-2-ylcarbamoyl)naphthalen-2-yl]-3-{1-1[3-{2-[(2-methoxyethyl)amino]ethoxy}-5,7-dimethyltricyclo[3.3.1.1^{3,7}]dec-1-yl)methyl]-5-methyl-1H-pyrazol-4-yl}pyridine-2-carboxylic acid (Compound W3.15)

### 1.15.1. 7-(6-(tert-butoxycarbonyl)-5-(1-((3-(2-(N-(2-methoxyethyl)-2-(trimethylsilyl)ethylsulfonamido)ethoxy)-5,7-dimethyladamantan-1-yl)methyl)-5-methyl-1H-pyrazol-4-yl)pyridin-2-yl)-1-naphthoic acid

To a solution of methyl 7-(4,4,5,5-tetramethyl-1,3,2-dioxaborolan-2-yl)-1-naphthoate (208 mg) in dioxane (10 mL) and water (5 mL) was added Example 1.14.6 (500 mg). (Ph₃P)₂PdCl₂ (45.6 mg) and CsF (296 mg). The mixture was stirred at 120° C. for 30 minutes under microwave conditions (Biotage Initiator), diluted with ethyl acetate and washed with water and brine. The organic layer was dried over Na₂SO₄, filtered, and concentrated. The residue was purified by flash chromatography, eluting with 20% ethyl acetate in dichloromethane, to give the ester intermediate. The ester was dissolved in a mixture of tetrahydrofuran (10 mL), methanol (5 mL) and H₂O (5 mL) and treated with lithium hydroxide monohydrate (200 mg). The mixture was stirred at room temperature for 4 hours, acidified with 1N aqueous HCl solution and diluted with ethyl acetate (300 mL). After washing with water ad brine, the organic layer was dried over Na₂SO₄. After filtration, evaporation of the solvent afforded the title compound. MS (ESI) m/e 888.20 (M+H)⁺.

### 1.15.2. 6-[8-(1,3-benzothiazol-2-ylcarbamoyl)naphthalen-2-yl]-3-{1-[3-{2-[(2-methoxyethyl)amino]ethoxy}-5,7-dimethyltricyclo[3.3.1.1]dec-1-yl)methyl]-5-methyl-1H-pyrazol-4-yl}pyridine-2-carboxylic acid

To a solution of Example 1.15.1 (500 mg) in dichloromethane (10 mL) was added benzo[d]thiazol-2-amine (85 mg). 1-ethyl-3-[3-(dimethylamino)propy l]-carbodiimide hydrochloride (216 mg) and 4-(dimethylamino)pyridine (138 mg). The mixture was stirred at room temperature overnight, diluted with ethyl acetate, and washed with water and brine. The organic layer was then dried over Na2SO₄, filtered, and concentrated to dryness. The residue was dissolved in tetrahydrofuran (10 mL) and treated with tetrabutyl ammonium fluoride (10 mL, 1M in tetrahydrofuran) overnight. The reaction mixture was diluted with ethyl acetate and washed with water and brine. The organic layer was dried over Na₂SO₄, filtered, and concentrated to dryness. The residue was dissolved in dichloromethane (5 mL) and treated with trifluoroacetic acid (5 mL) overnight. The mixture was then concentrated and the residue was purified by reverse HPLC (Gilson), eluting with 10-85% acetonitrile in 0.1% TFA in water, to give the title compound. ¹H NMR (400 MHz, dimethyl sulfoxide-d₆) δ ppm 13.11 (s, 1H), 9.00 (s, 1H), 8.60-8.29 (m, 3H), 8.26-8.13 (m, 3H), 8.03 (ddd, 2H), 7.92 (d, 1H), 7.80 (d, 1H), 7.74-7.62 (m, 1H), 7.51-7.42 (m, 2H), 7.36 (td, 1H), 3.88 (s, 2H), 3.61-3.52 (m, 2H), 3.27 (s, 3H), 3.17-2.95 (m, 4H), 2.22 (s, 3H), 1.43 (s, 2H), 1.30 (q, 4H), 1.23-0.96 (m, 6H), 0.86 (s, 6H). MS (ESI) m/e 799.2 (M+H)⁺.

### 1.16. Synthesis of 6-[8-(1,3-benzothiazol-2-ylcarbamoyl)-3,4-dihydroisoquinolin-2(1H)-yl]-3-[1-({3,5-dimethyl-7-[2-(oxetan-3-ylamino)ethoxy]tricyclo[3.3.1.1^{3,7}]dec-1-yl}methyl)-5-methyl-1H-pyrazol-4-yl]pyridine-2-carboxylic acid (Compound W3.16)

### 1.16.1, methyl 2-(5-bromo-6-(tert-butoxycarbonyl)pyridin-2-yl)-1,2,3,4-tetrahydroisoquinoline-8-carboxylate

To a solution of methyl 1,2,3,4-tetrahydroisoquinoline-8-carboxylate hydrochloride (12.37 g) and Example 1.4.4 (15 g) in dimethyl sulfoxide (100 mL) was added N,N-diisopropylethylamine(12 mL). The mixture was stirred at 50° C. for 24 hours. The mixture was diluted with ethyl acetate (500 mL), washed with water and brine, and dried over Na₂SO₄. After filtration and evaporation of the solvent, the crude material was purified via silica gel column chromatography, eluting with 20% ethyl acetate in hexane, to give the title compound. MS (ESI) m/e 448.4 (M+H)⁺.

### 1.16.2, methyl 2-(6-(tert-butoxycarbonyl)-5-(4,4,5,5-tetramethyl-1,3,2-dioxaborolan-2-yl)pyridin-2-yl)-1,2,3,4-tetrahydroisoquinoline-8-carboxylate

To a solution of Example 1.16.1 (2.25 g) and [1,1′-bis(diphenylphosphino)ferrocene]dichloropalladium(II) (205 mg) in acetonitrile (30 mL) was added triethylamine (3 mL) and pinacolborane (2 mL). The mixture was stirred at reflux for 3 hours. The mixture was diluted with ethyl acetate (200 mL), washed with water and brine, and dried over Na₂SO₄. Filtration, evaporation of the solvent, and silica gel chromatography (eluting with 20% ethyl acetate in hexane) gave the title compound. MS (ESI) m/e 495.4 (M+H)⁺.

### 1.163, methyl 2-(6-(tert-butoxycarbonyl)-5-(1-((3-(2-((tert-butoxycarbonyl)amino)ethoxy)-5,7-dimethyladamantan-1-yl)methyl)-5-methyl-1H-pyrazol-4-yl)pyridin-2-yl)-1,2,3,4-tetrahydroisoquinoline-8-carboxylate

To a solution of Example 1.16.2 (4.94 g) in tetrahydrofuran (60 mL) and water (20 mL) was added Example 1.4.2 (5.57 g), 1,3,5,7-tetramethyl-8-tetradecyl-2,4,6-trioxa-8-phosphaadamantane (412 mg), tris(dibenzylideneacetone)dipalladium(0) (457 mg), and K₃PO₄ (11 g). The mixture was stirred at reflux overnight. The reaction mixture was diluted with ethyl acetate (500 mL), washed with water and brine, and dried over Na₂SO₄. After filtration and evaporation of the solvent the crude material was purified via column chromatography, eluting with 20% ethyl acetate in heptane, to give the title compound. MS (ESI) m/e 784.4 (M+H)⁺.

### 1.16.4. 2-(6-(tert-butoxycarbonyl)-5-(1-((3-(2-((tert-butoxycarbonyl)amino)ethoxy)-5,7-dimethyladamantan-1-yl)methyl)-5-methyl-1H-pyrazol-4-yl)pyridin-2-yl)-1,2,3,4-tetrahydroisoquinoline-8-carboxylic acid

To a solution of Example 1.16.3 (10 g) in tetrahydrofuran (60 mL), methanol (30 mL) and water (30 mL), was added lithium hydroxide monohydrate (1.2 g). The mixture was stirred at room temperature for 24 hours. The reaction mixture was neutralized with 2% aqueous HCl and concentrated under vacuum. The residue was diluted with ethyl acetate (800 mL), washed with water and brine, and dried over Na₂SO₄. Filtration and evaporation of the solvent gave the title compound. MS (ESI) m/e 770.4 (M+H)⁺.

### 1.16.5, tert-butyl 6(8-(benzo[d]thiazol-2-ylcarbamoyl)-3,4-dihydroisoquinolin-2(1H)-yl)-3-(1-((3-(2-((tert-butoxycarbonyl)amino)ethoxy)-5,7-dimethyladamantan-1-yl)methyl)-5-methyl-1H-pyrazol-4-yl)picolinate

To a solution of Example 1.16.4 (3.69 g) in N,N-dimethylformamide (20 mL) was added benzo[d]thiazol-2-amine (1.1 g), fluoro-N,N,N′,N′-tetramethylformamidinium hexafluorophosphate (1.9 g) and N,N diisopropylethylamine (1.86 g). The mixture was stirred at 60° C. for 3 hours. The reaction mixture was diluted with ethyl acetate (500 mL), washed with water and brine, and dried over Na₂SO₄. Filtration, evaporation of the solvent, and column purification (20% ethyl acetate in heptane) gave the title compound. MS (ESI) m/e 902.2(M+H)⁺.

### 1.16.6. 3-(1-((3-(2-aminoethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid

Example 1.16.5 (2 g) was dissolved in 5(0% TFA in dichloromethane (20 mL) and stirred overnight. The solvents were removed under vacuum and the residue was loaded on a reverse-phase column and eluted with 20-80% acetonitrile in water (0.1% TFA) to give the title compound. MS (ESI) m/e 746.3 (M+H)⁺.

### 1.16.7. 6-[8-(1,3-benzothiazol-2-ylcarbamoyl)-3,4-dihydroisoquinolin-2(1H)-yl]-3-[1-({3,5-dimethyl-7-[2-(oxetan-3-ylamino)ethoxy]tricyclo[3.3.1.1^{3,7}]dec-1-yl}methyl)-5-methyl-1H-pyrazol-4-yl]pyridine-2-carboxylic acid

A solution of Example 1.16.6 (0.050 g), oxetan-3-one (5 mg) and sodium triacetoxyborohydride (0.018 g) was stirred together in dichloromethane (1 mL) at room temperature. After stirring for 1 hour, additional oxetan-3-one (5 mg) and sodium triacetoxyborohydride (0.018 g) were added and the reaction was stirred overnight. The reaction was concentrated, dissolved in a 1:1 mixture of dimethyl sulfoxide/methanol (2 mL) and purified by HPLC using a Gilson system (20-60% acetonitrile in water containing 0.1% v/v trifluoroacetic acid). The desired fractions were combined and freeze-dried to provide the title compound. ¹H NMR (400 MHz, dimethyl sulfoxide-d₆) δ ppm 12.95 (s, 1H), 9.26 (s, 2H), 8.12 (d, 1H), 7.88 (d, 1H), 7.71 (d, 1H), 7.63-7.50 (m, 3H), 7.50-7.41 (m, 2H), 7.38 (s, 1H), 7.05 (d, 1H), 5.05 (s, 2H), 4.79 (t, 2H), 4.68 (dd, 2H), 4.54-4.41 (m, 1H), 3.98 (t, 2H), 3.92 (s, 2H), 3.63 (t, 2H), 3.16-3.04 (m, 4H), 2.20 (s, 3H), 1.52 (s, 2H), 1.47-1.06 (m, 10H), 0.96 (s, 61f). MS (ESI) m/e 802.2 (M+H)⁺.

### 1.17. Synthesis of 6-[6-(3-aminopyrrolidin-1-yl)-8-(1,3-benzothiazol-2-ylcarbamoyl)-3,4-dihydroisoquinolin-2(1H)-yl]-3-(1-{[3-(2-methoxyethoxy)-5,7-dimethyltricyclo[3.3.1.1^{3,7}]dec-1-yl]methyl}-5-methyl-1H-pyrazol-4-yl)pyridine-2-carboxylic acid (Compound W3.17)

### 1.17.1. 4-iodo-1-((3-(2-methoxyethoxy)-5,7-dimethyladamantan-1-yl)methyl)-5-methyl-1H-pyrazole

Example 1.1.6 (3.00 g) was dissolved in 1,4-dioxane (40 mL), and sodium hydride (60% in mineral oil, 568 mg) was added. The solution was mixed at room temperature for 15 minutes, and methyl iodide (1.64 mL) was added. The solution was stirred at room temperature for three days, and then 0.01 M aqueous HCl solution (50 mL) was added. The solution was extracted with diethyl ether three times. The combined organic extracts were washed with brine and dried on anhydrous sodium sulfate. After filtration, the solvent was removed under reduced pressure and then under high vacuum to yield the title compound. MS (ESI) m/e 459 (M+H)⁺.

### 1.17.2, benzyl 4-oxopent-2-ynoate

Benzyl 4-hydroxypent-2-ynoate (40.5 g) and Dess-Martin Periodinane (93.0 g) in dichloromethane (500 mL) were stirred for 1 hour at 0° C. The solution was poured into diethyl ether (IL), and the combined organics were washed three times with 1M aqueous NaOH and brine, dried over Na₂SO₄, filtered, and concentrated. The residue was chromatographed on silica gel using 5% ethyl acetate in heptanes to give the title compound.

### 1.17.3. (S)-benzyl 6-(3-((tert-butoxycarbonyl)amino)pyrrolidin-1-yl)-2-(2,2,2-trifluoroacetyl)-1,2,3,4-tetrahydroisoquinoline-8-carboxylate

A solution of 1-(2,2,2-trifluoroacetyl)piperidin-4-one (6.29 g), (S)-tert-butyl pyrrolidin-3-ylcarbamate (6.0 g), and p-toluenesulfonic acid monohydrate (0.613 g) in ethanol (80 mL) was stirred for 1 hour at room temperature. Example 1.17.2 (6.51 g) was then added and the reaction was stirred for 24 hours at room temperature, and heated to 45° C. for 3 days. The reaction was then cooled and poured into diethyl ether (600 mL). The resulting solution was washed twice with water and brine, dried over Na₂SO₄, filtered, and concentrated. The residue was chromatographed on silica gel using 5-50% ethyl acetate in heptanes to give the product.

### 1.17.4. (S)-benzyl 6-(3-((tert-butoxycarbonyl)amino)pyrrolidin-1-yl)-1,2,3,4-tetrahydroisoquinoline-8-carboxylate

A solution of Example 1.17.3 (3.1 g) and potassium carbonate (1.8 g) in a mixture of tetrahydrofuran (30 mL), methanol (10 mL), and water (25 mL) was stirred for 48 hours at 45° C. The reaction was then cooled and diluted with dichloromethane (3(00) mL). The layers were separated and the organic layer was dried over Na₂SO₄, filtered, and concentrated to give the title compound.

### 1.17.5. (S)-benzyl 2-(5-bromo-6-(tert-butoxycarbonyl)pyridin-2-yl)-6-(3-((tert-butoxycarbonyl)amino)pyrrolidin-1-yl)-1,2,3,4-tetrahydroisoquinoline-8-carboxylate

A solution of Example 1.17.4 (1.6 g). Example 1.4.4 (1.08 g), and triethylamine (0.59 mL) in N,N-dimethylformamide (10 mL) was heated to 50° C. for 24 hours. The reaction was cooled and poured into ethyl acetate (400 mL). The resulting solution was washed three times with water and brine, dried over Na₂SO₄, filtered, and concentrated. The residue was chromatographed on silica gel using 5-50% ethyl acetate in heptanes to give the product.

### 1.17.6. (S)-benzyl 2-(6-(tert-butoxycarbonyl)-5-(4,4,5,5-tetramethyl-1,3,2-dioxaborolan-yl)pyridin-2-yl)-6-(3-((tert-butoxycarbonyl)amino)pyrrolidin-1-yl)-1,2,3,4-tetrahydroisoquinoline-8-carboxylate

A solution of Example 1.17.5 (500 mg), 4,4,5,5-tetramethyl-1,3,2-dioxaborolane (136 mg), and triethylamine (0.200 mL) in acetonitrile (5 mL) was heated to 75° C. for 24 hours. The reaction was allowed to cool to room temperature and concentrated to dryness. The crude material was then purified via column chromatography, eluting with 5-50% ethyl acetate in heptanes, to give the title compound.

### 1.17.7, benzyl 2-(6-(tert-butoxycarbonyl)-5-(1-((3-(2-methoxyethoxy)-5,7-dimethyladamantan-1-yl)methyl)-5-methyl-1H-pyrazol-4-yl)pyridin-2-yl)-6-((S)-3-((tert-butoxycarbonyl)amino)pyrrolidin-1-yl)-1,2,3,4-tetrahydroisoquinoline-8-carboxylate

A solution of Example 1.17.6 (240 mg), Example 1.17.1 (146 mg), 1.3,5,7-tetramethyl-8-tetradecyl-2,4,6-trioxa-8-phosphaadamantane (13 mg), palladium (II)acetate (14.6 mg), and tripotassium phosphate (270 mg) in dioxane (7 mL) and water (3 mL) was heated to 70° C. for 24 hours. The reaction was allowed to cool to room temperature and was concentrated to dryness. The crude material was then purified via column chromatography, eluting with 5-25% ethyl acetate in heptanes, to give the title compound.

### 1.17.8. 2-(6-(tert-butoxycarbonyl)-5-(1-((3-(2-methoxyethoxy)-5,7-dimethyladamantan-1-yl)methyl)-5-methyl-1H-pyrazol-4-yl)pyridin-2-yl)-6-((S)-3-((tert-butoxycarbonyl)amino)pyrrolidin-1-yl)-1,2,3,4-tetrahydroisoquinoline-8-carboxylic acid

A solution of Example 1.17.7 (1.6 g) and lithium hydroxide monohydrate (5 mg) in a 3:1:1 mixture of tetrahydrofuran/methanol/water (10 mL) was stirred for 4 days. The reaction was acidified with 1M aqueous HCl solution and poured into ethyl acetate (150 mL). The resulting solution was washed with brine, dried over Na₂SO₄, filtered, and concentrated to give the title compound.

### 1.17.9, tert-butyl 6-(8-(benzo[d]thiazol-2-ylcarbamoyl)-6-((S)-3-((tert-butoxycarbonyl)amino)pyrrolidin-1-yl)-3,4-dihydroisoquinolin-2(1H)-yl)-3-(1-((3-(2-methoxyethoxy)-5,7-dimethyladamantan-1-yl)methyl)-5-methyl-1H-pyrazol-4-yl)picolinate

A solution of Example 1.17.8 (78 mg), benzo[d]thiazol-2-amine (16 mg), 0-(7-azabenzotriazol-1-yl)-N,N,N′,N′-tetramethyluronium hexafluorophosphate (48 mg), and diisopropylethylamine (0.024 mL) in N,N-dimethylformamide (3 mL) was heated to 50° C. for 48 hours. The reaction was then cooled and poured into ethyl acetate (100 mL). The resulting solution was washed three times with water and brine, dried over Na₂SO₄, filtered, and concentrated. The residue was purified via column chromatography, eluting with 20-100% ethyl acetate in heptanes, to give the title compound.

### 1.17.10. 6-[6-(3-aminopyrrolidin-1-yl)-8-(1,3-benzothiazol-2-ylcarbamoyl)-3,4-dihydroisoquinolin-2(1H)-yl]-3-(1-{[3-(2-methoxyethoxy)-5,7-dimethyltricyclo[3.3.1.1^{3,7}]dec-1-yl]methyl}-5-methyl-1H-pyrazol-4-yl)pyridine-2-carboxylic acid

Example 1.17.9 (40 mg) in dichloromethane (3 mL) was treated with trifluoroacetic acid (2 mL) overnight. The mixture was concentrated to provide the title compound as a TFA salt. MS (ESI) m/e 845.7 (M+H)⁺.

### 1.18. Synthesis of 6-[8-(1,3-benzothiazol-2-ylcarbamoyl)-3,4-dihydroisoquinolin-2(1H)-yl]-3-{1-[(3,5-dimethyl-7-{2-[(2-sulfamoylethyl)amino]ethoxy}tricyclo[3.3.1.1^{3,7}]dec-1-yl)methyl]-5-methyl-1H-pyrazol-4-yl}pyridine-2-carboxylic acid (Compound W3.18)

### 1.18.1. 3-bromo-5,7-dimethyladamantanecarboxylic acid

Into a 50 mL round-bottomed flask at 0° C., was added bromine (16 mL). Iron powder (7 g) was added, and the reaction was stirred at 0° C. for 30 minutes. 3,5-Dimethyladamantane-1-carboxylic acid (12 g) was added. The mixture was warmed up to room temperature and stirred for 3 days. A mixture of ice and concentrated HCl was poured into the reaction mixture. The resulting suspension was treated twice with Na₂SO₃ (50 g in 200 mL water) and extracted three times with dichloromethane. The combined organics were washed with 1N aqueous HCl, dried over sodium sulfate, filtered, and concentrated to give the title compound.

### 1.18.2. 3-bromo-5,7-dimethyladamantanemethanol

To a solution of Example 1.18.1 (15.4 g) in tetrahydrofuran (200 mL) was added BH₃ (1M in tetrahydrofuran, 150 mL), and the mixture was stirred at room temperature overnight. The reaction mixture was then carefully quenched by adding methanol dropwise. The mixture was then concentrated under vacuum, and the residue was balanced between ethyl acetate (500 mL) and 2N aqueous HCl (100 mL). The aqueous layer was further extracted twice with ethyl acetate, and the combined organic extracts were washed with water and brine, dried over sodium sulfate, and filtered. Evaporation of the solvent gave the title compound.

### 1.18.3. 1-((3-bromo-5,7-dimethyltricyclo[3.3.1.1^{3,7}]dec-1-yl)methyl)-1H-pyrazole

To a solution of Example 1.18.2 (8.0 g) in toluene (60 mL) was added 1H-pyrazole (1.55 g) and cyanomethylenetributylphosphorane (2.0 g), and the mixture was stirred at 90° C. overnight. The reaction mixture was concentrated, and the residue was purified by silica gel column chromatography (10:1 heptane:ethyl acetate) to give the title compound. MS (ESI) m/e 324.2 (M+H)⁺.

### 1.18.4. 2-{[3,5-dimethyl-7-(1H-pyrazol-1-ylmethyl)tricyclo[3.3.1.1^{3,7}]dec-1-yl]oxy}ethanol

To a solution of Example 1.18.3 (4.0 g) in ethane-1,2-diol (12 mL) was added triethylamine (3 mL). The mixture was stirred at 150° C. under microwave conditions (Biotage Initiator) for 45 minutes. The mixture was poured into water (100 mL) and extracted three times with ethyl acetate. The combined organic extracts were washed with water and brine, dried over sodium sulfate, and filtered. Evaporation of the solvent gave a residue that was purified by silica gel chromatography, eluting with 20% ethyl acetate in heptane, followed by 5% methanol in dichloromethane, to give the title compound. MS (ESI) m/e 305.2 (M+H)⁺.

### 1.18.5. 2-({3,5-dimethyl-7-[5-methyl-1H-pyrazol-1-yl)methyl]tricyclo[3.3.1.1^{3,7}]dec-1-yl}oxy)ethanol

To a cooled (−78° C.) solution of Example 1.18.4 (6.05 g) in tetrahydrofuran (100 mL) was added n-BuLi (40 mL, 2.5M in hexane), and the mixture was stirred at −78° C. for 1.5 hours. Iodomethane (10 mL) was added through a syringe, and the mixture was stirred at −78° C. for 3 hours. The reaction mixture was then quenched with aqueous NH₄Cl and extracted twice with ethyl acetate, and the combined organic extracts were washed with water and brine. After drying over sodium sulfate, the solution was filtered and concentrated, and the residue was purified by silica gel column chromatography, eluting with 5% methanol in dichloromethane, to give the title compound. MS (ESI) m/e 319.5 (M+H)⁺.

### 1.18.6. 1-({3,5-dimethyl-7-[2-(hydroxy)ethoxy]tricyclo[3.3.1.1^{3,7}]dec-1-yl}methyl)-4-iodo-5-methyl-1H-pyrazole

To a solution of Example 1.18.5 (3.5 g) in N,N-dimethylformamide (30 mL) was added N-iodosuccinimide (3.2 g), and the mixture was stirred at room temperature for 1.5 hours. The reaction mixture was diluted with ethyl acetate (600 mL) and washed with aqueous NaHSO₃, water and brine. The organic layer was dried over sodium sulfate, filtered and concentrated under reduced pressure. The residue was purified by silica gel chromatography, eluting with 20% ethyl acetate in dichloromethane, to give the title compound. MS (ESI) m/e 445.3 (M+H)⁺.

### 1.18.7. 1-((3-(2-((tert-butyldimethylsilyl)oxy)ethoxy)-5,7-dimethyladamantan-1-yl)methyl)-4-iodo-5-methyl-1H-pyrazole

Tert-butyldimethylsilyl trifluoromethanesulfonate (5.34 mL) was added to a solution of Example 1.18.6 (8.6 g) and 2,6-lutidine (3.16 mL) in dichloromethane (125 mL) at −40° C., and the reaction was allowed to warm to room temperature overnight. The mixture was concentrated, and the residue was purified by silica gel chromatography, eluting with 5-20% ethyl acetate in heptanes, to give the title compound. MS (ESI) m/e 523.4 (M+H)⁺.

### 1.18.8. 1-((3-(2-((tert-butyldimethylsilyl)oxy)ethoxy)-5,7-dimethyladamantan-1-yl)methyl)-5-methyl-4-(4,4,5,5-tetramethyl-1,3,2-dioxaborolan-2-yl)-1H-pyrazole

n-Butyllithium (8.42 mL, 2.5M in hexanes) was added to Example 1.18.7 (9.8 g) in 120 mL tetrahydrofuran at −78° C., and the reaction was stirred for 1 minute. Trimethyl borate (3.92 mL) was added, and the reaction stirred for 5 minutes. Pinacol (6.22 g) was added, and the reaction was allowed to warm to room temperature and was stirred 2 hours. The reaction was quenched with pH 7 buffer, and the mixture was poured into ether. The layers were separated, and the organic layer was concentrated under reduced pressure. The residue was purified by silica gel chromatography, eluting with 1-25% ethyl acetate in heptanes, to give the title compound.

### 1.18.9. 6-fluoro-3-bromopicolinic acid

A slurry of 6-amino-3-bromopicolinic acid (25 g) in 400 mL 1:1 dichloromethane/chloroform was added to nitrosonium tetrafluoroborate (18.2 g) in dichloromethane (100 mL) at 5° C. over 1 hour. The resulting mixture was stirred for another 30 minutes, then warmed to 35° C., and stirred overnight. The reaction was cooled to room temperature, and then adjusted to pH 4 with aqueous NaH₂PO₄ solution. The resulting solution was extracted three times with dichloromethane, and the combined extracts were washed with brine, dried over sodium sulfate, filtered and concentrated to provide the title compound.

### 1.18.10. Tert-butyl 3-bromo-6-fluoropicolinate

Para-toluenesulfonyl chloride (27.6 g) was added to a solution of Example 1.18.9 (14.5 g) and pyridine (26.7 mL) in dichloromethane (100 mL) and tert-butanol (80 mL) at 0° C. The reaction was stirred for 15 minutes, and then warmed to room temperature, and stirred overnight. The solution was concentrated and partitioned between ethyl acetate and aqueous Na₂CO₃ solution. The layers were separated, and the aqueous layer extracted with ethyl acetate. The organic layers were combined, rinsed with aqueous Na₂CO₃ solution and brine, dried over sodium sulfate, filtered, and concentrated to provide the title compound.

### 1.18.11, methyl 2-(5-bromo-6-(tert-butoxycarbonyl)pyridin-2-yl)-1,2,3,4-tetrahydroisoquinoline-8-carboxylate

To a solution of methyl 1,2,3,4-tetrahydroisoquinoline-8-carboxylate hydrochloride (12.37 g) and Example 1.18.10 (15 g) in dimethyl sulfoxide (100 mL) was added N,N-diisopropylethylamine (12 mL), and the mixture was stirred at 50 (C for 24 hours. The mixture was then diluted with ethyl acetate (500 mL) and washed with water and brine. The organic layer was dried over sodium sulfate, filtered and concentrated under reduced pressure. The residue was purified by silica gel chromatography, eluting with 20% ethyl acetate in hexane, to give the title compound. MS (ESI) m/e 448.4 (M+H)⁺.

### 1.18.12, methyl 2-(6-(tert-butoxycarbonyl)-5-(4,4,5,5-tetramethyl-1,3,2-dioxaborolan-2-yl)pyridin-2-yl)-1,2,3,4-tetrahydroisoquinoline-8-carboxylate

To a solution of Example 1.18.11 (2.25 g) and [1,1′-bis(diphenylphosphino)ferrocene]dichloropalladium(II) (205 mg) in acetonitrile (30 mL) was added triethylamine (3 mL) and pinacolborane (2 mL), and the mixture was stirred at reflux for 3 hours. The mixture was diluted with ethyl acetate (200 mL) and washed with water and brine. The organic layer was dried over sodium sulfate, filtered and concentrated under reduced pressure. Purification of the residue by silica gel chromatography, eluting with 20% ethyl acetate in hexane, provided the title compound.

### 1.18.13, methyl 2-(6-(tert-butoxycarbonyl)-5-(1-((3-(2-hydroxyethoxy)-5,7-dimethyladamantan-1-yl)methyl)-5-methyl-1H-pyrazol-4-yl)pyridin-2-yl)-1,2,3,4-tetrahydroisoquinoline-8-carboxylate

To a solution of Example 1.18.12 (2.25 g) in tetrahydrofuran (30 mL) and water (10 mL) was added Example 1.18.6 (2.0 g), 1,3,5,7-tetramethyl-6-phenyl-2,4,8-trioxa-6-phosphaadamantane (329 mg), tris(dibenzylideneacetone)dipalladium(0) (206 mg) and potassium phosphate tribasic (4.78 g). The mixture was refluxed overnight, cooled and diluted with ethyl acetate (500 mL). The resulting mixture was washed with water and brine, and the organic layer was dried over sodium sulfate, filtered and concentrated. The residue was purified by flash chromatography, eluting with 20% ethyl acetate in heptanes followed by 5% methanol in dichloromethane, to provide the title compound.

### 1.18.14, methyl 2-(6-(tert-butoxycarbonyl)-5-(1-((3,5-dimethyl-7-(2-((methylsulfonyl)oxy)ethoxy)adamantan-1-yl)methyl)-5-methyl-1H-pyrazol-4-yl)pyridin-2-yl)-1,2,3,4-tetrahydroisoquinoline-8-carboxylate

To a cold solution of Example 1.18.13 (3.32 g) in dichloromethane (100 mL) in an ice-bath was sequentially added triethylamine (3 mL) and methanesulfonyl chloride (1.1 g). The reaction mixture was stirred at room temperature for 1.5 hours and diluted with ethyl acetate, and washed with water and brine. The organic layer was dried over sodium sulfate, filtered, and concentrated to provide the title compound.

### 1.18.15, methyl 2-(5-(1-((3-(2-azidoethoxy)-5,7-dimethyladamantan-1-yl)methyl)-5-methyl-1H-pyrazol-4-yl)-6-(tert-butoxycarbonyl)pyridin-2-yl)-1,2,3,4-tetrahydroisoquinoline-8-carboxylate

To a solution of Example 1.18.14 (16.5 g) in N,N-dimethylformamide (120 mL) was added sodium azide (4.22 g). The mixture was heated at 80° C. for 3 hours, cooled, diluted with ethyl acetate and washed with water and brine. The organic layer was dried over sodium sulfate, filtered, and concentrated. The residue was purified by flash chromatography, eluting with 20% ethyl acetate in heptanes, to provide the title compound.

### 1.18.16. 2-(5-(1-((3-(2-azidoethoxy)-5,7-dimethyladamantan-1-yl)methyl)-5-methyl-1H-pyrazol-4-yl)-6-(tert-butoxycarbonyl)pyridin-2-yl)-1,2,3,4-tetrahydroisoquinoline-8-carboxylic acid

To a solution of Example 1.18.15 (10 g) in a mixture of tetrahydrofuran (60 mL), methanol (30 mL) and water (30 mL) was added lithium hydroxide monohydrate (1.2 g). The mixture was stirred at room temperature overnight and neutralized with 2% aqueous HCl. The resulting mixture was concentrated, and the residue was dissolved in ethyl acetate (800 mL), and washed with brine. The organic layer was dried over sodium sulfate, filtered, and concentrated to provide the title compound.

### 1.18.17, tert-butyl 3-(1-((3-(2-azidoethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinate

A mixture of Example 1.18.16 (10 g), benzo[d]thiazol-2-amine (3.24 g), fluoro-N,N,N′,N′-tetramethylformamidinium hexafluorophosphate (5.69 g) and N,N-diisopropylethylamine (5.57 g) in N,N-dimethylformamide (20 mL) was heated at 60° C. for 3 hours, cooled and diluted with ethyl acetate. The resulting mixture was washed with water and brine. The organic layer was dried over sodium sulfate, filtered, and concentrated. The residue was purified by flash chromatography, eluting with 20% ethyl acetate in dichloromethane to give the title compound.

### 1.18.18, tert-butyl 3-(1-(((3-(2-aminoethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinate

To a solution of Example 1.18.17 (2.0 g) in tetrahydrofuran (30 mL) was added Pd/C (10%, 200 mg). The mixture was stirred under a hydrogen atmosphere overnight. The insoluble material was filtered off and the filtrate was concentrated to provide the title compound.

### 1.18.19. 3-(1-((3-(2-aminoethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin—2(1H)-yl)picolinic acid

Example 1.18.18 (200 mg) in dichloromethane (2.5 mL) was treated with trifluoroacetic acid (2.5 mL) overnight. The reaction mixture was concentrated, and the residue was purified by reverse phase chromatography (C18 column), eluting with 20-60% acetonitrile in water containing 0.1% v/v trifluoroacetic acid, to provide the title compound. MS (ESI) m/e 746.2 (M+H)⁺.

### 1.18.20. 6-[8-(1,3-benzothiazol-2-ylcarbamoyl)-3,4-dihydroisoquinolin-2(1H)-yl]-3-{1-[(3,5-dimethyl-7-{2-[(2-sulfamoylethyl)amino]ethoxy}tricyclo[3.3.1.1^{3,7}]dec-1-yl)methyl]-5-methyl-1H-pyrazol-4-yl}pyridine-2-carboxylic acid

A mixture of Example 1.18.19 (18 mg) and ethenesulfonamide (5.2 mg) in N,N-dimethylformamide (I mL) and water (0.3 mL) was stirred for one week. The mixture was purified by reverse phase chromatography (C18 column), eluting with 20-60% acetonitrile in water containing 0.1% v/v trifluoroacetic acid, to provide the title compound. ¹H NMR (500 MHz, dimethyl sulfoxide-d₆) δ ppm 8.03 (d, 1H), 7.79 (d, 1H), 7.61 (d, 1H), 7.45-7.50 (m, 1H), 7.41-7.44 (m, 1H), 7.33-7.39 (m, 3H), 7.23 (s, 1H), 6.73 (d, 1H), 4.87 (s, 2H), 3.89 (t, 2H), 3.79 (s, 2H), 3.12-3.20 (m, 2H), 2.99 (t, 2H), 2.85 (s, 2H), 2.09 (s, 3H), 1.32 (dd, 4H), 1.08-1.19 (m, 5H), 1.04 (d, 4H), 0.86 (s, 6H). MS (ESI) m/e 853.2(M+H)⁺.

### 1.19 Synthesis of 3-(1-[3-(2-aminoethoxy)-5,7-dimethyltricyclo[3.3.1.1^{3,7}]dec-1-yl]methyl)-5-methyl-1H-pyrazol-4-yl)-6-[3-(1,3-benzothiazol-2-ylcarbamoyl)-6,7-dihydrothieno[3,2-c]pyridin-5(4H)-yl]pyridine-2-carboxylic acid (W3.19)

### 1.19.1 6,7-dihydro-4H-thieno[3,2-c]pyridine-3,5-dicarboxylic acid 5-tert-butyl ester 3-methyl ester

Tert-butyl 3-bromo-6,7-dihydrothieno[3,2-c]pyridine-5(4H)-carboxylate (1000 mg) and dichloro[1,1′-bis(diphenylphosphino)ferrocene]palladium(II) (69 mg) were placed in a 50 mL pressure bottle, and methanol (20 mL) was added, followed by trimethylamine (636 mg). The solution was degassed and flushed with argon three times. The solution was then degassed and flushed with carbon monoxide and heated to 100° C. for 18 hours under 60 psi of carbon monoxide. The solvent was removed under reduced pressure, and the residue was purified by flash column chromatography on silica gel, eluting with 50% ethyl acetate in heptanes. The solvent was removed under reduced pressure to yield the title compound.

### 1.19.2 4,5,6,7-tetrahydro-thieno[3,2-c]pyridine-3-carboxylic acid methyl ester

Example 1.19.1 (940 mg) was dissolved in dichloromethane (12 mL). Trifluoroacetic acid (2220 mg) was added, and the solution was stirred for three hours. The solvent was removed under reduced pressure to yield the title compound as the trifluoroacetic acid salt, which was used without further purification.

### 1.19.3 5-(5-bromo-6-tert-butoxycarbonyl-pyridin-2-yl)-4,5,6,7-tetrahydro-thieno[3,2-c]pyridine-3-carboxylic acid methyl ester

The title compound was prepared by substituting Example 1.19.2 for ethyl 5,6,7,8-tetrahydroimidazo[1,5-a]pyrazine-1-carboxylate hydrochloride in Example 1.4.5. MS (ESI) m/e 452, 450 (M+H)⁺.

### 1.19.4 5-[6-tert-butoxycarbonyl-5-(4,4,5,5-tetramethyl-[1,3,2]dioxaborolan-2-yl)-pyridin-2-yl]-4,5,6,7-tetrahydro-thieno[3,2-c]pyridine-3-carboxylic acid methyl ester

The title compound was prepared by substituting Example 1.19.3 for Example 1.1.9 in Example 1.1.10. MS (ESI) m/e 500 (M+H)⁺, 531 (M+CH₃OH—H)⁻.

### 1.19.5 5-(6-tert-butoxycarbonyl-5-{1-[5-(2-tert-butoxycarbonylamino-ethoxy)-3,7-dimethyl-adamantan-1-ylmethyl]-5-methyl-1H-pyrazol-4-yl}-pyridin-2-yl)-4),6,7-tetrahydro-thieno[3,2-c]pyridine-3-carboxylic acid methyl ester

The title compound was prepared by substituting Example 1, 19.4 for Example 1.4.6 in Example 1.4.7.

### 1.19.6 5-(6-tert-butoxycarbonyl-5-(1-[5-(2-tert-butoxycarbonylamino-ethoxy)-3,7-dimethyl-adamantan-1-ylmethyl]-5-methyl-1H-pyrazol-4-yl)-pyridin-2-yl)-4,5,6,7-tetrahydro-thieno[3,2-c]pyridine-3-carboxylic acid

The title compound was prepared by substituting Example 1.19.5 for Example 1.4.7 in Example 1.4.8. MS (ESI) m/e 776 (M+H)⁺, 774 (M−H)⁻.

### 1.19.7 6-[3-(benzothiazol-2-ylcarbamoyl)-6,7-dihydro-4H-thieno[3,2-c]pyridin-5-yl]-3-(1-[5-(2-tert-butoxycarbonylamino-ethoxy)-3,7-dimethyl-adamantan-1-ylmethyl]-5-methyl-1H-pyrazol-4-yl)-pyridine-2-carboxylic acid tert-butyl ester

The title compound was prepared by substituting Example 1.19.6 for Example 1.4.8 in Example 1.4.9. MS (ESI) m/e 892 (M+H)⁺, 890 (M−H)⁻.

### 1.19.8 3-(1-{[3-(2-aminoethoxy)-5,7-dimethyltricyclo[3.3.1.13,7]dec-1-yl]methyl}-5-methyl-1H-pyrazol-4-yl)-6-[3-(1,3-benzothiazol-2-ylcarbamoyl)-6,7-dihydrothieno[3,2-c]pyridin-5(4H)-yl]pyridine-2-carboxylic acid

The title compound was prepared by substituting Example 1.19.7 for Example 1.1.13 in Example 1.1.14. ¹H NMR (400 MHz, dimethyl sulfoxide-d_{d}) δ ppm 8.11 (bs, 1H), 8.00 (d, 1H), 7.77 (d, 1H), 7.68 (bs, 3H), 7.53 (d, 1H), 7.47 (t, 1H), 7.36-7.31 (m, 2H), 7.14 (d, 1H), 4.71 (s, 2H), 3.99 (t, 2H), 3.85 (s, 2H), 3.52 (m, 2H), 3.00 (t, 2H), 2.91 (q, 2H), 2.13 (s, 3H), 1.44 (s, 2H), 1.31 (q, 4H), 1.16 (m, 4H), 1.05 (q, 2H), 0.88 (s, 6H). MS (ESI) m/e 752 (M+H)⁺, 750 (M−H)⁻.

### 1.20 Synthesis of 3-(1-{([3-(2-aminoethoxy)-5,7-dimethyltricyclo[3.3.1.13,7]dec-1-yl]methyl}-5-methyl-1H-pyrazol-4-yl)-6-[1-(1,3-benzothiazol-2-ylcarbamoyl)-3-(trifluoromethyl)-5,6-dihydroimidazo[1,5-a]pyrazin-7(8H)-yl]pyridine-2-carboxylic acid (W3.20)

### 1.20.1 7-(5-bromo-6-tert-butoxycarbonyl-pyridin-2-yl)-3-trifluoromethyl-5,6,7,8-tetrahydro-imidazo[1,5-a]pyrazine-1-carboxylic acid methyl ester

The title compound was prepared by substituting methyl 3-(trifluoromethyl)-5,6,7,8-tetrahydroimidazo[1,5-a]pyrazine-1-carboxylate for ethyl 5,6,7,8-tetrahydroimidazo[1,5-a]pyrazine-1-carboxylate hydrochloride in Example 1.4.5. MS (ESI) m/e 449 (M-tBu+H)⁺, 503 (M−H)⁻.

### 1.20.2 7-[6-tert-butoxycarbonyl-5-(4,4,5,5-tetramethyl-[1,3,2]dioxaborolan-2-yl)-pyridin-2-yl]-3-trifluoromethyl-5,6,7,8-tetrahydro-imidazo[1,5-a]pyrazine-1-carboxylic acid methyl ester

The title compound was prepared by substituting Example 1.20.1 for Example 1, 1.9 in Example 1.1.10. MS (ESI) m/e 553 (M+H)⁺.

### 1.20.3 di-tert-butyl[2-({3-[(4-iodo-5-methyl-1H-pyrazol-1-yl)methyl]-5,7-dimethyltricyclo[3.3.1.13,7]decan-1-yl}oxy)ethyl]-2-imidodicarbonate

Example 1.1.6 (5.000 g) was dissolved in dichloromethane (50 mL). Triethylamine (1.543 g) was added, and the solution was cooled on an ice bath. Methanesulfonyl chloride (1.691 g) was added dropwise. The solution was allowed to warm to room temperature and stir for 30 minutes. Saturated aqueous sodium bicarbonate solution (50 mL) was added. The layers were separated, and the organic layer was washed with brine (50 mL). The aqueous portions were then combined and back extracted with dichloromethane (50 mL). The organic portions were combined, dried over anhydrous sodium sulfate, filtered, and concentrated. The residue was dissolved in acetonitrile (50 mL). Di-tert-butyl iminodicarboxylate (2.689 g) and cesium carbonate (7.332 g) were added, and the solution was refluxed for 16 hours. The solution was cooled and added to diethyl ether (100 mL) and water (100 mL). The layers were separated. The organic portion was washed with brine (50 mL). The aqueous portions were then combined and back extracted with diethyl ether (100 mL). The organic portions were combined, dried over anhydrous sodium sulfate, filtered, and concentrated under reduced pressure. The material was purified by flash column chromatography on silica gel, eluting with 20% ethyl acetate in heptanes. The solvent was evaporated under reduced pressure to provide the title compound. MS (ESI) m/e 666 (M+Na)⁺.

### 1.20.4 methyl 7-(6-(tert-butoxycarbonyl)-5-(1-((3-(2-(di-(tert-butoxycarbonyl)amino)ethoxy)-5,7-dimethyladamantan-1-yl)methyl)-5-methyl-1H-pyrazol-4-yl)pyridin-2-yl)-3-(trifluoromethyl)-5,6,7,8-tetrahydroimidazo[1,5-a]pyrazine-1-carboxylate

The title compound was prepared by substituting Example 1.20.2 for Example 1.4.6 and Example 1.20.3 for Example 1.4.2 in Example 1.4.7. MS (ESI) m/e 964 (M+Na)⁺, 940 (M−H)⁻.

### 1.20.5 7-(6-(tert-butoxycarbonyl)-5-(1-((3-(2-(di-(tert-butoxycarbonyl)amino)ethoxy)-5,7-dimethyladamantan-1-yl)methyl)-5-methyl-1H-pyrazol-4-yl)pyridin-2-yl)-3-(trifluoromethyl)-5,6,7,8-tetrahydroimidazo[1,5-a]pyrazine-1-carboxylic acid

The title compound was prepared by substituting Example 1.20.4 for Example 1.4.7 in Example 1.4.8. MS (ESI) m/e 828 (M+H)⁺, 826 (M−H)⁻.

### 1.20.6 tert-butyl 6-(1-(benzo[d]thiazol-2-ylcarbamoyl)-3-(trifluoromethyl)-5,6-dihydroimidazo[1,5-a]pyrazin-7(8H)-yl)-3-(1-((3-(2-(di-(tert-butoxycarbonyl)amino)ethoxy)-5,7-dimethyladamantan-1-yl)methyl)-5-methyl-1H-pyrazol-4-yl)picolinate

The title compound was prepared by substituting Example 1.20.5 for Example 1.4.8 in Example 1.4.9. MS (ESI) m/e 1058 (M−H)⁻.

### 1.20.7 3-(1-{[3-(2-aminoethoxy)-5,7-dimethyltricyclo[3.3.1.13,7]dec-1-yl]methyl}-5-methyl-1H-pyrazol-4-yl)-6-[1-(1,3-benzothiazol-2-ylcarbamoyl)-3-(trifluoromethyl)-5,6-dihydroimidazo[1,5-a]pyrazin-7(8H)-yl]pyridine-2-carboxylic acid

The title compound was prepared by substituting Example 1.20.6 for Example 1.1.13 in Example 1.1.14. ¹H NMR (400 MHz, dimethyl sulfoxide-d₆) δ ppm 11.99 (bs, 1H), 8.00 (d, 1H), 7.79 (d, 1H), 7.66 (bs, 3H), 7.61 (d, 1H), 7.47 (t, 1H), 7.35 (t, 2H), 7.19 (d, 1H), 5.20 (s, 2H), 4.37 (t, 2H), 4.16 (t, 2H), 3.86 (s, 2H), 3.51 (t, 2H), 2.91 (q, 2H), 2.14 (s, 3H), 1.44 (s, 2H), 1.36-1.24 (m, 4H), 1.19-1.02 (m, 6H), 0.88 (s, 6H). MS (ESI) m/e 804 (M+H)⁺, 802 (M−H)⁻.

### 1.21 Synthesis of 6-[8-(1,3-benzothiazol-2-ylcarbamoyl)-6-{methyl[2-(methylamino)ethyl]amino}-3,4-dihydroisoquinolin-2(1H)-yl]-3-(1-{[3-(2-methoxyethoxy)-5,7-dimethyltricyclo[3.3.1.13,7]dec-1-yl]methyl}-5-methyl-1H-pyrazol-4-yl)pyridine-2-carboxylic acid (W3.21)

### 1.21.1 methyl 3-bromo-5-(bromomethyl)benzoate

AIBN (2,2′-azobis(2-methylpropionitrile)) (1.79 g) was added to methyl 3-bromo-5-methylbenzoate (50 g) and N-bromosuccinimide (44.7 g) in 350 mL acetonitrile, and the mixture was refluxed overnight. An additional 11 g of N-bromosuccinimide and 0.5 g of AIBN (2,2′-azobis(2-methylpropionitrile)) was added, and the refluxing was continued for 3 hours. The mixture was concentrated, and then taken up in 500 mL ether, and stirred for 30 minutes. The mixture was then filtered, and the resulting solution was concentrated. The crude product was chromatographed on silica gel using 10% ethyl acetate in heptane to give the title compound.

### 1.21.2 methyl 3-bromo-5-(cyanomethyl)benzoate

Tetrabutylammonium cyanide (50 g) was added to Example 1.21.1 (67.1 g) in 300 mL acetonitrile, and the mixture was heated to 70° C. overnight. The mixture was cooled, poured into diethyl ether, and rinsed with water and brine. The mixture was concentrated and chromatographed on silica gel using 2-20% ethyl acetate in heptane to give the title compound.

### 1.21.3 methyl 3-(2-aminoethyl)-5-bromobenzoate

Borane-tetrahydrofuran complex (126 mL, 1M solution) was added to a solution of Example 1.21.2 (16 g) in 200 mL tetrahydrofuran, and the mixture was stirred overnight. The reaction was carefully quenched with methanol (50 mL), and then concentrated to 50 mL volume. The mixture was then taken up in 120 mL methanol/120 mL 4M HCl/120 mL dioxane, and stirred overnight. The organics were removed by evaporation under reduced pressure, and the residue was extracted with diethyl ether (2×). The organic extracts were discarded. The aqueous layer was basified with solid K₂CO₃, and then extracted with ethyl acetate, and dichloromethane (2×). The extracts were combined, dried over Na₂SO₄, filtered and concentrated to give the title compound.

### 1.21.4 methyl 3-bromo-5-(2(2-(2,2,2-trifluoroacetamido)ethyl)benzoate

Trifluoroacetic anhydride (9.52 mL) was added dropwise to a mixture of Example 1.21.3 (14.5 g) and triethylamine (11.74 mL) in 200 mL dichloromethane at 0° C. Upon addition, the mixture was allowed to warm to room temperature and was stirred for three days. The mixture was poured into diethyl ether, and washed with NaHCO₃ solution and brine. The mixture was concentrated and chromatographed on silica gel using 5-30% ethyl acetate in heptanes to give the title compound.

### 1.21.5 methyl 6-bromo-2-(2,2,2-trifluoroacetyl)-1,2,3,4-tetrahydroisoquinoline-8-carboxylate

Sulfuric acid was added to Example 1.21.4 (10 g) until it went into solution (40 mL), at which time paraformaldehyde (4.24 g) was added, and the mixture was stirred for 2 hours. The solution was then poured onto 400 mL ice, and stirred 10 minutes. It was then extracted with ethyl acetate (3×), and the combined extracts were washed with NaHCO₃ solution and brine, and then concentrated The crude product was chromatographed on silica gel using 2-15% ethyl acetate in heptanes to give the title compound.

### 1.21.6 methyl 6-((2-((tert-butoxycarbonyl)(methyl)amino)ethyl)(methyl)amino)-2-(22,2-trifluoroacetyl)-1,2,3,4-tetrahydroisoquinoline-8-carboxylate

Example 1.21.5 (2.25 g), tert-butyl methyl(2-(methylamino)ethyl)carbamate (1.27 g), palladium (II) acetate (0.083 g), 4,5-bis(diphenylphosphino)-9,9-dimethylxanthene (0.213 g) and cesium carbonate (4.00 g) were stirred in 40 mL dioxane at 80° C. overnight. The mixture was concentrated and chromatographed on silica gel using 5-50% ethyl acetate in heptanes to give the title compound.

### 1.21.7 methyl 2-(5-bromo-6-(tert-butoxycarbonyl)pyridin-2-yl)-6-((2-((tert-butoxycarbonyl)(methyl)amino)ethyl)(methyl)amino)-1,2,3,4-tetrahydroisoquinoline-8-carboxylate

Example 1.21.6 (3 g) and potassium carbonate (2.63 g) were stirred in 30 mL tetrahydrofuran, 20 mL methanol, and 25 mL water overnight. The mixture was concentrated and 60 mL N,N-dimethylformamide was added. To this was then added Example 1.4.4 (1.08 g) and triethylamine (0.6 mL), and the reaction was stirred at 50° C. overnight. The mixture was cooled to room temperature and poured into ethyl acetate (200 mL). The solution was washed with water (3×) and brine, then dried over Na₂SO₄, filtered, and concentrated. The residue was chromatographed on silica gel using 5-50% ethyl acetate in heptanes to give the title compound. MS (ESI) m/e 635 (M+H)⁺.

### 1.21.8 methyl 6-((2-((tert-butoxycarbonyl)(methyl)amino)ethyl)methyl)amino)-2-(6-(tert-butoxycarbonyl)-5-(4,4,5,5-tetramethyl-1,3,2-dioxaborolan-2-yl)pyridin-2-yl)-1,2,3,4-tetrahydroisoquinoline-8-carboxylate

The title compound was prepared by substituting Example 1.21.7 for Example 1.1.9 in Example 1.1.10.

### 1.21.9 methyl 6-((2-((tert-butoxycarbonyl)(methyl)amino)ethyl)(methyl)amino)-2-(6-(tert-butoxycarbonyl)-5-(1-((3-(2-methoxyethoxy)-5,7-dimethyladamantan-1-yl)methyl)-5-methyl-1H-pyrazol-4-yl)pyridin-2-yl)-1,2,3,4-tetrahydroisoquinoline-8-carboxylate

The title compound was prepared by substituting Example 1.21.8 for Example 1.5.11 and Example 1.17.1 for Example 1.5.10 in Example 1.5.12. MS (ESI) m/e 885.6 (M+H)⁺.

### 1.21.10 6-((2-((tert-butoxycarbonylmethyl)amino)ethyl)(methyl)amino)-2-(6-(tert-butoxycarbonyl)-5-(1-((3-(2-methoxyethoxy)-5,7-dimethyladamantan-1-yl)methyl)-5-methyl-1H-pyrazol-4-yl)pyridin-2-yl)-1,2,3,4-tetrahydroisoquinoline-8-carboxylic acid

The title compound was prepared by substituting Example 1.21.9 for Example 1.4.7 in Example 1.4.8.

### 1.21.11 tert-butyl 6-(8-(benzo[d]thiazol-2-ylcarbamoyl)-6-((2-((tert-butoxycarbonyl)(methyl)amino)ethyl)(methyl)amino)-3,4-dihydroisoquinolin-2(1H)-yl)-3-(1-((3-(2-methoxyethoxy)-5,7-dimethyladamantan-1-yl)methyl)-5-methyl-1H-pyrazol-4-yl)picolinate

The title compound was prepared by substituting Example 1.21.10 for Example 1.4.8 in Example 1.4.9. MS (ESI) m/e 1003.6 (M+H)⁺.

### 1.21.12 6-[8-(1,3-benzothiazol-2-ylcarbamoyl)-6-(methyl[2-(methylamino)ethyl]amino)-3,4-dihydroisoquinolin-2(l H)-yl]-3-(1-{[3-(2-methoxyethoxy)-5,7-dimethyltricyclo[3.3.1.13,7] dec-1-yl]methyl}-5-methyl-1H-pyrazol-4-yl)pyridine-2-carboxylic acid

Example 1.21.11 (40 mg) was stirred in 2 mL trifluoroacetic acid and 3 mL dichloromethane overnight. After evaporation of the solvent, the residue was purified on an HPLC (Gilson system, eluting with 10-85% acetonitrile in 0.1% trifluoroacetic acid in water) to give the title compound. ¹H NMR (400 MHz, dimethyl sulfoxide-d₆) δ ppm 12.75 (bs, 1H), 12.50 (br s, 1H), 8.40 (m, 2H), 8.01 (d, 1H), 7.76 (d, 1H), 7.45 (m, 2H), 7.32 (t, 1H), 7.24 (s, 1H), 6.99 (d, 1H), 6.86 (d, 1H), 6.78 (d, 1H), 4.72 (m, 2H), 3.98 (m, 2H), 3.80 (m, 4H), 3.76 (s, 2H), 3.55 (m, 2H), 3.29 (d, 3H), 3.20 (s, 3H), 3.15 (m, 2H), 2.90 (s, 3H), 2.58 (t, 2H), 2.05 (s, 3H), 1.30 (s, 2H), 1.21 (m, 4H), 1.08 (m, 4H), 0.98 (m, 2H), 0.85 (s, 6H). MS (ESI) m/e 847.5 (M+H)⁺.

### 1.22 Synthesis of 6-[8-(1,3-benzothiazol-2-ylcarbamoyl)-6-methoxy-3,4-dihydroisoquinolin-2(1H)-yl]-3-[1-({3,5-dimethyl-7-[2-(methylamino)ethoxy]tricyclo[0.3.1.13,7]dec-1-yl}methyl)-5-methyl-1H-pyrazol-4-yl]pyridine-2-carboxylic acid (W3.22)

### 1.22.1 methyl 6-(4,4,5,5-tetramethyl-1,32-dioxaborolan-2-yl)-2-(2,2,2-trifluoroacetyl)-1,2,3,4-tetrahydroisoquinoline-8-carboxylate

A mixture of Example 1.21.5 (4.5 g), 4,4,4′,4′,5,5,5′,5′-octamethyl-2,2′-bi(1,3,2-dioxaborolane) (3.75 g), [1,1′-bis(diphenylphosphino)ferrocene]dichloropalladium(II) dichloromethane (0.4 g), and potassium acetate (3.62 g) was stirred in 60 mL dioxane at 70° C. for 24 hours. The mixture was then diluted with ethyl acetate, and rinsed with water and brine. The mixture was concentrated and chromatographed on silica gel using 5-50% ethyl acetate in heptanes to give the title compound.

### 1.22.2 methyl 6-hydroxy-2-(2,2,2-trifluoroacetyl)-1,2,3,4-tetrahydroisoquinoline-8-carboxylate

Hydrogen peroxide (30%, 1.1 mL) was added to a mixture of Example 1.22.1 (4 g) and 1M aqueous NaOH solution (9.86 mL) in 40 mL tetrahydrofuran and 40 mL water, and the mixture was stirred for 90 minutes. The solution was acidified with concentrated HCl, and extracted twice with ethyl acetate. The combined extracts were washed with brine. The mixture was then concentrated and chromatographed on silica gel using 5-50% ethyl acetate in heptanes to give the title compound. MS (ESI) m/e 304.2 (M+H)⁺.

### 1.22.3 methyl 6-methoxy-2-(2,2,2-trifluoroacetyl)-1,2,3,4-tetrahydroisoquinoline-8-carboxylate

Trimethylsilyldiazomethane (2.6 mL, 2M solution in diethyl ether) was added to Example 1.22.2 (8(00) mg) in 10 mL methanol, and the reaction was stirred for 24 hours. The mixture was then concentrated and chromatographed on silica gel using 5-25% ethyl acetate in heptanes to give the title compound. MS (ESI) m/e 318.2 (M+H)⁺.

### 1.22.4 methyl 2-(5-brom-6-(tert-butoxcarbonyl)pyridin-2-yl)-6-methoxy-1,2,3,4-tetrahydroisoquinoline-8-carboxylate

The title compound was prepared by substituting Example 1.22.3 for Example 1.21.6 in Example 1.21.7. MS (ESI) m/e 479.1 (M+H)⁺.

### 1.22.5 methyl 2-(6-(tert-butoxycarbonyl)-5-(4,4,5,5-tetramethyl-1,3,2-dioxaborolan-2-yl)pyridin-2-yl)-6-methoxy-1,2,3,4-tetrahydroisoquinoline-8-carboxylate

The title compound was prepared by substituting Example 1.22.4 for Example 1.1.9 in Example 1.1.10. MS (ESI) m/e 525.1 (M+H)⁺.

### 1.22.6 methyl 2-(6-(tert-butoxycarbonyl)-5-(1-((-(2-((tert-butoxycarbonyl)(methyl)amino)ethoxy)-5,7-dimethyladamantan-1-yl)methyl)-5-methyl-1H-pyrazol-4-yl)pyridin-2-yl)-6-methoxy-1,2,3,4-tetrahydroisoquinoline-8-carboxylate

The title compound was prepared by substituting Example 1.22.5 for Example 1.5.11 and Example 1.1.9 for Example 1.5.10 in Example 1.5.12. MS (ESI) m/e 829.6 (M+H)⁺.

### 1.22.7 2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-6-methoxy-1,2,3,4-tetrahydroisoquinoline-8-carboxylic acid

The title compound was prepared by substituting Example 1.22.6 for Example 1.4.7 in Example 1.4.8. MS (ESI) m/e 814.6 (M+H)⁺.

### 1.22.8 tert-butyl 6-(8-(benzo[d]thiazol-2-ylcarbamoyl)-6-methoxy-3,4-dihydroisoquinolin-2(1H)-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

The title compound was prepared by substituting Example 1.22.7 for Example 1.4.8 in Example 1.4.9. MS (ESI) m/e 946.5 (M+H)⁺.

### 1.22.9 6-[8-(1,3-benzothiazol-2-ylcarbamoyl)-6-methoxy-3,4-dihydroisoquinolin-2(1H)-yl]-3-[1-((3,5-dimethyl-7-[2-(methylamino)ethoxy]tricyclo[3.3.1.13,7]dec-1-yl)methyl)-5-methyl-1H-pyrazol-4-yl]pyridine-2-carboxylic acid

The title compound was prepared by substituting Example 1.22.8 for Example 1.21.11 in Example 1.21.12. ¹H NMR (400 MHz, dimethyl sulfoxide-d₆) δ ppm 12.75 (bs, 1H), 12.50 (br s, 1H), 8.21 (m, 2H), 8.01 (d, 1H), 7.76 (d, 1H), 7.44 (m, 2H), 7.32 (t, 1H), 7.25 (s, 1H), 7.20 (d, 1H), 6.99 (d, 1H), 6.90 (d, 1H), 4.72 (m, 2H), 3.80 (m, 4H), 3.55 (s, 3H), 3.50 (d, 3H), 2.98 (m, 4H), 2.51 (t, 2H), 2.05 (s, 3H), 1.35 (s, 2H), 1.26 (m, 4H), 1.10 (m, 4H), 1.00 (m, 2H), 0.85 (s, 6H). MS (ESI) m/e 790.4 (M+H)⁺.

### 1.23 Synthesis of 3-(1-{([3-(2-aminoethoxy)-5,7-dimethyltricyclo[3.3.1.13,7]dec-1-y]methyl}-5-methyl-1H-pyrazol-4-yl)-6-[4-(1,3-benzothiazol-2-ylcarbamoyl)quinolin-6-yl]pyridine-2-carboxylic acid (W3.23)

### 1.23.1 ethyl 6-(4,4,5,5-tetramethyl-1,3,2-dioxaborolan-2-yl)quinoline-4-carboxylate

To a solution of ethyl 6-bromoquinoline-4-carboxylate (140 mg) in N,N-dimethylformamide (2 mL) was added [1,1′-bis(diphenylphosphino)ferrocene]dichloropalladium(II) dichloromethane (20 mg), potassium acetate (147 mg) and bis(pinacolato)diboron (190 mg). The mixture was stirred at 60° C. overnight. The mixture was cooled to room temperature and used in the next reaction directly. MS (ESI) m/e 328.1 (M+H)⁺.

### 1.23.2di-tert-butyl (2-[(3,5-dimethyl-7-{[5-methyl-4-(4,4,5,5-tetramethyl-1,3,2-dioxaborolan-2-yl)-1H-pyrazol-1-yl]methyl)tricyclo[3.3.1.13,7]decan-1-yl)oxy]ethyl}-2-imidodicarbonate

To a solution of Example 1.20.3 (13 g) in dioxane (100 mL) was added dicyclohexyl(2′,6′-dimethoxy-[1.1′-biphenyl]-2-yl)phosphine (S-Phos) (1.0 g) and bis(benzonitrile)palladium(II) chloride (0.23 g) and the reaction was purged with several house vacuum/N₂ refills. 4,4,5,5-Tetramethyl-1,3,2-dioxaborolane (8.8 mL) and triethylamine (8.4 mL) was added followed by a couple more house vacuum/nitrogen refills and then the reaction was heated to 85° C. under nitrogen for 90 minutes. The reaction was cooled, filtered through diatomaceous earth and rinsed with methyl tert-butyl ether. The solution was then concentrated and chromatographed on silica gel using 25% ethyl acetate in heptanes to give the title compound.

### 1.23.3 tert-butyl 3-(1-[(3-{2-[bis(tert-butoxycarbonyl)amino]ethoxy}-5,7-dimethyltricyclo[3.3.1.13,7]decan-1-yl)methyl]-5-methyl-1H-pyrazol-4-yl)-6-chloropyridine-2-carboxylate

To a solution of Example 1.23.2 (12.3 g) and tert-butyl 3-bromo-6-chloropicolinate (5.9 g) in dioxane (50 mL) was added (1S,3R,5R,7S)-1,3,5,7-tetramethyl-8-phenyl-2,4,6-trioxa-8-phosphaadamantane(CyTop) (0.52 g) and bis(dibenzylideneacetone)palladium(0) (0.66 g). After several house vacuum/nitrogen refills, potassium phosphate (4.06 g) and water (25 mL) were added and the reaction was heated at 80° C. under nitrogen for 30 minutes. The reaction was cooled and then water and ethyl acetate were added. The organic layer was separated and washed with brine. The combined aqueous layers were extracted with ethyl acetate, and dried over sodium sulfate. The solution was filtered, concentrated and chromatographed on silica gel using 33% ethyl acetate in heptanes to give the title compound.

### 1.23.4 ethyl 6-[5-(1-[(3-(2-[bis(tert-butoxycarbonyl)amino]ethoxy)-5,7-dimethyltricyclo[3.3.1.13,7]dec-1-yl)methyl]-5-methyl-1H-pyrazol-4-yl-6-(tert-butoxycarbonyl)pyridin-2-yl]quinoline-4-carboxylate

To a solution of Example 1.23.1 (164 mg) in 1,4-dioxane (10 mL) and water (5 mL) was added Example 1.23.3 (365 mg), bis(triphenylphosphine)palladium(II) dichloride (35 mg), and CsF (228 mg). The mixture was stirred at 120° C. for 30 minutes under microwave conditions (Biotage Initiator). The mixture was diluted with ethyl acetate (200 mL) and washed with water and brine and dried over anhydrous sodium sulfate. Filtration and evaporation of the solvent gave a residue that purified by silica gel chromatography, eluting with 20% ethyl acetate in heptane, to give the title compound. MS (ESI) m/e 894.3(M+H)⁺.

### 1.23.5 6-(6-(tert-butoxycarbonyl)-5-(1-((3-(2-((tert-butoxycarbonyl)amino)ethoxy)-5,7-dimethyladamantan-1-yl)methyl)-5-methyl-1H-pyrazol-4-yl)pyridin-2-yl)quinoline-4-carboxylic acid

To a solution of Example 1.23.4 (3.1 g) in tetrahydrofuran (20 mL), methanol (10 mL) and water (10 mL) was added LiOH H₂O (240 mg). The mixture was stirred at room temperature overnight. The mixture was acidified with aqueous 2N HCl, diluted with ethyl acetate (400 mL), washed with water and brine, and dried over anhydrous sodium sulfate. Filtration and evaporation of the solvent gave the title compound, which was used without further purification. MS (ESI) m/e 766.3(M+H)⁺.

### 1.23.6 3-(1-{[3-(2-aminoethoxy)-5,7-dimethyltricyclo[3.3.1.13,7]dec-1-yl]methyl}-5-methyl-1H-pyrazol-4-yl)-6-[4-(1,3-benzothiazol-2-ylcarbamoyl)quinolin-6-yl]pyridine-2-carboxylic acid

To a solution of Example 1.23.5 (4.2 g) in dichloromethane (30 mL) was added benzo[d]thiazol-2-amine (728 mg), 1-ethyl-3-[3-(dimethylamino)propyl]-carbodiimide hydrochloride (1.40 g) and 4-(dimethylamino)pyridine (890 mg). The mixture was stirred at room temperature overnight. The reaction mixture was diluted with ethyl acetate (500 mL), washed with water and brine, dried over anhydrous sodium sulfate, filtered and concentrated under reduced pressure. The residue was dissolved in dichloromethane and trifluoroacetic acid (10 mL, 1:1) and stirred overnight. The solvents were removed under reduced pressure. The residue was diluted with N,N-dimethylformamide (2 mL), filtered and purified by reverse-phase HPLC on a Gilson system (C18 column), eluting with 20-80% acetonitrile in water containing 0.1% trifluoroacetic acid, to give the title compound. ¹H NMR (400 MHz, dimethyl sulfoxide-d₆) δ ppm 9.12 (dd, 1H), 8.92 (s, 1H), 8.61 (dt, 1H), 8.35-8.16 (m, 2H), 8.07 (d, 1H), 7.97-7.87 (m, 2H), 7.81 (d, 1H), 7.66 (s, 3H), 7.53-7.44 (m, 2H), 7.38 (t, 1H), 3.88 (s, 2H), 3.49 (t, 2H), 2.89 (q, 2H), 2.22 (s, 4H), 1.43 (s, 2H), 1.29 (q, 4H), 1.15 (s, 4H), 1.09-0.96 (m, 2H), 0.86 (s, 7H). MS (ESI) m/e 742.2 (M+H)⁺.

### 1.24 Synthesis of 6-[5-amino-8-(1,3-benzothiazol-2-ylcarbamoyl)-3,4-dihydroisoquinolin-2(1H)-yl]-3-[1-({3,5-dimethyl-7-[2-(methylamino)ethoxy]tricyclo[3.3.1.13,7]dec-1-yl}methyl)-5-methyl-1H-pyrazol-4-yl]pyridine-2-carboxylic acid (W3.24)

### 1.24.1 5-tert-butoxycarbonylamino-2-(2,2,2-trifluoro-acetyl)-1,2,3,4-tetrahydr-isoquinoline-8-carboxylic acid methyl ester

Example 1.13.4 (5000 mg), tert-butyl carbamate (1920 mg), and cesium carbonate (6674 mg) were added to 1,4-dioxane (80 mL). The solution was degassed and flushed with nitrogen three times. Diacetoxypalladium (307 mg) and (9,9-dimethyl-9H-xanthene-4,5-diyl)bis(diphenylphosphine) (1580 mg) were added, and the solution was degassed and flushed with nitrogen once. The solution was heated to 80° C. for 16 hours. The solution was cooled, and 1 M aqueous HCl (150 mL) was added. The solution was extracted with 50% ethyl acetate in heptanes. The organic portion was washed with brine and dried on anhydrous sodium sulfate. The solution was filtered, concentrated and purified by flash column chromatography on silica gel, eluting with 30% ethyl acetate in heptanes. The solvent was removed under reduced pressure to yield the title compound. MS (ESI) m/e 420 (M+NH₄)⁺, 401 (M−H)⁻.

### 1.24.2 5-tert-butoxycarbonylamino-1,2,3,4-tetrahydro-isoquinoline-8-carboxylic acid methyl ester

The title compound was prepared by substituting Example 1.24.1 for Example 1.13.5 in Example 1.13.6. MS (ESI) m/e 307 (M+H)⁺, 305 (M−H)⁻.

### 1.24.3 2-(5-bromo-6-tert-butoxycarbonyl-pyridin-2-yl)-5-tert-butoxycarbonylamino-1,2,3,4-tetrahydro-isoquinoline-8-carboxylic acid methyl ester

The title compound was prepared by substituting Example 1.24.2 for Example 1.13.6 in Example 1.13.7. MS (ESI) m/e 562, 560 (M+H)⁺, 560, 558 (M−H)⁻.

### 1.24.4 5-tert-butoxycarbonylamino-2-[6-tert-butoxycarbonyl-5-(4,4,5,5-tetramethyl-[1,3,2]dioxaborolan-2-yl)-pyridin-2-yl]-1,2,3,4-tetrahydro-isoquinoline-8-carboxylic acid methyl ester

The title compound was prepared by substituting Example 1.24.3 for Example 1.13.7 in Example 1.13.8. MS (ESI) m/e 610 (M+H)⁺, 608 (M−H)⁻.

### 1.24.5 methyl 2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-5-((tert-butoxycarbonyl)amino)-1,2,3,4-tetrahydroisoquinoline-8-carboxylate

The title compound was prepared by substituting Example 1.24.4 for Example 1.13.8 and Example 1.1.9 for Example 1.4.2 in Example 1.13.9. MS (ESI) m/e 913 (M+H)⁺, 911 (M−H)⁻.

### 1.24.6 2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-5-((tert-butoxycarbonyl)amino)-1,2,3,4-tetrahydroisoquinoline-8-carboxylic acid

The title compound was prepared by substituting Example 1.24.5 for Example 1.13.9 in Example 1.13.10. MS (ESI) m/e 899 (M+H)⁺, 897 (M−H)⁻.

### 1.24.7 tert-butyl 6-(8-(benzo[d]thiazol-2-ylcarbamoyl)-5-((tert-butoxycarbonyl)amino)-3,4-dihydroisoquinolin-2(1H)-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

The title compound was prepared by substituting Example 1.24.6 for Example 1.13.10 in Example 1.13.11. MS (EST) m/e 1031 (M+H)⁺, 1029 (M−H)⁻.

### 1.24.8 6-[5-amino-8-(1,3-benzothiazol-2-ylcarbamoyl)-3,4-dihydroisoquinolin-2(1H)-yl]-3-[1-((3,5-dimethyl-7-[2-(methylamino)ethoxyl]tricyclo[3.3.1.13,7]dec-1-yl)methyl)-5-methyl-1H-pyrazol-4-yl]pyridine-2-carboxylic acid

The title compound was prepared by substituting Example 1.24.7 for Example 1.13.11 in Example 1.13.12. ¹H NMR (400 MHz, dimethyl sulfoxide-d₆) δ ppm 11.42 (s, 1H), 7.98 (d, 1H), 7.75 (d, 1H), 7.55 (d, 1H), 7.44 (t, 2H), 7.31 (t, 1H), 7.27 (s, 1H), 6.92 (d, 1H), 6.58 (d, 1H), 5.74 (s, 2H), 4.99 (s, 2H), 3.93 (t, 2H), 3.82 (s, 2H), 3.57 (s, 3H), 3.54 (m, 2H), 3.09 (q, 2H), 2.98 (bs, 2H), 2.11 (s, 3H), 1.35-1.04 (m, 12H), 0.87 (s, 6H). MS (ESI) m/e 775 (M+H)⁺.

### 1.25 Synthesis of 6-[8-(1,3-benzothiazol-2-ylcarbamoyl)-6-[3-(methylamino)prop-1-yn-1-yl]-3,4-dihydroisoquinolin-2(1H)-yl]-3(1-([3-(2-methoxyethoxy)-5,7-dimethyltricyclo[3.3.1.13,7]dec-1-yl]methyl)-5-methyl-1H-pyrazol-4-yl)pyridine-2-carboxylic acid (W3.25)

### 1.25.1 methyl 6-(3-((tert-butoxycarbonyl)(methyl)amino)prop-1-yn-1-yl)-2-(2,2,2-trifluoroacetyl)-1,2,3,4-tetrahydroisoquinoline-8-carboxylate

A solution of Example 1.21.5 (1.97 g), tert-butyl methyl(prop-2-yn-1-yl)carbamate (1 g), bis(triphenylphosphine)palladium(II) dichloride (0.19 g), CuI (0.041 g), and triethylamine (2.25 mL) in 20 mL dioxane was stirred at 50° C. overnight. The mixture was then concentrated and chromatographed on silica gel using 10-50% ethyl acetate in heptanes to give the title compound.

### 1.25.2 methyl 2-(5-bromo-6-(tert-butoxycarbonyl)pyridin-2-yl)-6-(3-((tert-butoxycarbonyl)(methyl)amino)prop-1-yn-1-yl)-1,2,3,4-tetrahydroisoquinoline-8-carboxylate

The title compound was prepared by substituting Example 1.25.1 for Example 1.21.6 in Example 1.21.7. MS (ESI) m/e 616 (M+H)⁺.

### 1.25.3 methyl 6-(3-((tert-butoxycarbonyl)(methyl)amino)prop-1-yn-1-yl)-2-(6-(tert-butoxycarbonyl)-5-(4,4,5,5-tetramethyl-1,3,2-dioxaborolan-2-yl)pyridin-2-yl)-1,2,3,4-tetrahydroisoquinoline-8-carboxylate

The title compound was prepared by substituting Example 1.25.2 for Example 1.1.9 in Example 1.1.10. MS (ESI) m/e 662.3 (M+H)⁺.

### 1.25.4 methyl 6-(3-((tert-butoxycarbonyl)(methyl)amino)prop-1-yn-1-yl)-2-(6-(tert-butoxycarbonyl)-5-(1-((3-(2-methoxyethoxy)-5,7-dimethyladamantan-1-yl)methyl)-5-methyl-1H-pyrazol-4-yl)pyridin-2-yl)-1,2,3,4-tetrahydroisoquinoline-8-carboxylate

The title compound was prepared by substituting Example 1.25.3 for Example 1.5.11 and Example 1.17.1 for Example 1.5.10 in Example 1.5.12.

### 1.25.5 6-(3-((tert-butoxycarbonyl)(methyl)amino)prop-1-yn-1-yl)-2-(6-(tert-butoxycarbonyl)-5-(1-((3-(2-methoxyethoxy)-5,7-dimethyladamantan-1-yl)methyl)-5-methyl-1H-pyrazol-4-yl)pyridin-2-yl)-1,2,3,4-tetrahydroisoquinoline-8-carboxylic acid

The title compound was prepared by substituting Example 1.25.4 for Example 1.4.7 in Example 1.4.8.

### 1.25.6 tert-butyl 6-(8-(benzo[d]thiazol-2-ylcarbamoyl)-6-(3-((tert-butoxycarbonyl)(methyl)amino)prop-1-yn-1-yl)-3,4-dihydroisoquinolin-2(1H)-yl)-3-(1-((3-(2-methoxyethoxy)-5,7-dimethyladamantan-1-yl)methyl)-5-methyl-1H-pyrazol-4-yl)picolinate

The title compound was prepared by substituting Example 1.25.5 for Example 1.4.8 in Example 1.4.9.

### 1.25.7 6-[8-(1,3-benzothiazol-2-ylcarbamoyl)-6-[3-(methylamino)prop-1-yn-1-yl]-3,4-dihydroisoquinolin-2(1H)-yl]-3-(1-([3-(2-methoxyethoxy)-5,7-dimethyltricyclo[3.3.1.13,7]dec-1-yl]methyl)-5-methyl-1H-pyrazol-4-yl)pyridine-2-carboxylic acid

The title compound was prepared by substituting Example 1.25.6 for Example 1.21.11 in Example 1.21.12. ¹H NMR (400 MHz, dimethyl sulfoxide-d₆) δ ppm 12.95 (bs, 1H), 8.70 (m, 1H), 8.02 (d, 1H), 7.77 (d, 1H), 7.74 (m, 1H), 7.47 (m, 2H), 7.34 (m, 2H), 7.24 (s, 1H), 6.95 (m, 1H), 6.78 (m, 1H), 4.92 (s, 2H), 4.28 (t, 21H), 3.95 (t, 2H), 3.40 (s, 3H), 3.30 (m, 2H), 3.20 (s, 3H), 3.00 (m, 2H), 2.57 (t, 2H), 2.07 (s, 3H), 1.85 (m, 2H), 1.29 (d, 2H), 1.10-1.24 (m, 10H), 0.85 (s, 6H),

### 1.26 Synthesis of 6-[4-(1,3-benzothiazol-2-ylcarbamoyl)isoquinolin-6-yl]-3-[1-({3,5-dimethyl-7-[2-(methylamino)ethoxy]tricyclo[3.3.1.13,7]dec-1-yl}methyl)-5-methyl-1H-pyrazol-4-yl]pyridine-2-carboxylic acid (W3.26)

### 1.26.1 methyl 2-(3-bromophenyl)-2-cyanoacetate

To a solution of 2-(3-bromophenyl)acetonitrile (5 g) in tetrahydrofuran (50 mL) was added sodium hydride (3.00 g) portion wise at 23° C. The mixture was heated to 50° C. for 20 minutes. Dimethyl carbonate (8.60 mL) was added dropwise. The mixture was heated at reflux for 2 hours. The mixture was poured into cold and slightly acidic water. The aqueous layer was extracted with ethyl acetate (2×200 mL). The combined organic layers were washed with brine, dried over anhydrous sodium sulfate, filtered through a Büchner funnel and concentrated to give a residue, which was purified by silica gel column chromatography, eluting with 0%-25% dichloromethane/petroleum ether to afford the title compound. MS (LC-MS) m/e 256.0 (M+H)⁺

### 1.26.2 methyl 3-amino-2-(3-bromophenyl)propanoate

Sodium borohydride (14.89 g, 394 mmol) was added portionwise to a solution of Example 1.26.1 (10 g) and cobalt(II) chloride hexahydrate (18.73 g) in methanol (200 mL) at −20° C. The mixture was stirred for 1 hour and the pH was adjusted to 3 with 2N aqueous HCl. The mixture was concentrated. The residue was basified with 2 M aqueous sodium hydroxide and extracted with ethyl acetate. The combined organic layers were dried over anhydrous sodium sulfate, filtered and concentrated to provide the title compound. MS (LC-MS) m/e 260.0 (M+H)⁺.

### 1.26.3 methyl 2-(3-bromophenyl)-3-formamidopropanoate

A solution of Example 1.26.2 (3.6 g) in ethyl formate (54 mL) was heated at 80° C. for 5 hours. The solvent was removed, and the residue was purified by silica gel column chromatography eluting with petroleum/ethyl acetate (2:1-1:2) to give the title compound. MS (LC-MS) m/e 288.0 (M+H)⁺.

### 1.26.4 methyl 8-bromo-2,3-dioxo-3,5,6,10b-tetrahydro-2H-oxazolo[2,3-a]isoquinoline-6-carboxylate

Oxalyl chloride (1.901 mL) was slowly added to a solution of Example 1.26.3 (5.65 g) in dichloromethane (190 mL). The resulting mixture was stirred at 20° C. for 2 hours. The mixture was cooled to −20° C., and iron(III) chloride (3.84 g) was added. The resulting mixture was stirred at 20° C. for 3 hours. Aqueous hydrochloric acid (2M, 45 mL) was added in one portion, and the resulting biphasic mixture was vigorously stirred for 0.5 hours at room temperature. The biphasic mixture was poured into a separatory funnel, and the phases were separated. The organic layer was washed with brine, dried with sodium sulfate, and filtered. The solvent was evaporated under reduced pressure to provide the title compound. The crude product was directly used in subsequent step without purification. MS (LC-MS) me 342.0 (M+H)⁺.

### 1.26.5 methyl 6-bromo-3,4-dihydroisoquinoline-4-carboxylate

Example 1.26.4 (13.0 g) in methanol (345 mL) and sulfuric acid (23 mL) was heated at 80° C. for 16 hours. The mixture was concentrated, and the residue was diluted with water, basified with saturated aqueous sodium bicarbonate solution and extracted with ethyl acetate. The combined organic layers were washed with brine, dried over anhydrous sodium sulfate, filtered and concentrated. The residue was purified by silica gel column chromatography, eluting with petroleum ether/ethyl acetate (2:1-1:2) to give the title compound. MS (LC-MS) me 268.0 (M+H)⁺.

### 1.26.6 methyl 6-bromoisoquinoline-4-carboxylate

To a solution of Example 1.26.5 (5.25 g) in 1,4-dioxane (200 mL) at 60° C., was added manganese(IV) dioxide (8.5 g). The mixture was heated to 110° C. for 3 hours. The reaction mixture was filtered through a pad of diatomaceous earth and washed with dichloromethane and ethyl acetate. The filtrate was concentrated to dryness. The crude material was adsorbed onto silica gel and purified by silica gel chromatography, eluting with 5-30% ethyl acetate in dichloromethane to give the title compound. MS (LC-MS) m/e 267.9 (M+H)⁺.

### 1.26.7 methyl 6-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)isoquinoline-4-carboxylate

Example 1.26.6 (229 mg), 4,4,4′,4′,5,5,5′,5′-octamethyl-2,2′-bi(1,3,2-dioxaborolane) (328 mg) and potassium acetate (253 mg) in N,N-dimethylformamide (5 mL) was purged with N₂ for 5 minutes and [1,1′-bis(diphenylphosphino)ferrocene]dichloropalladium(II) dichloromethane (42.2 mg) was added. The mixture was heated at 100° C. overnight and cooled. To the mixture was added Example 1.1.11 (0.369 g), dichlorobis(triphenylphosphine)palladium(II) (0.060 g), cesium fluoride (0.261 g) and water (2 mL). The resulting mixture was heated at 100° C. for 10 hours and filtered. The filtrate was concentrated. The residue was dissolved in dimethyl sulfoxide and purified by reverse-phase HPLC on a Gilson system (C18 column), eluting with 20-80% acetonitrile in water containing 0.1% trifluoroacetic acid, to give the title compound. MS (ESI) m/e 794.5 (M+H)⁺.

### 1.26.8 6-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)isoquinoline-4-carboxylic acid

Example 1.26.7 (220 mg) in tetrahydrofuran-methanol was treated with 1 M aqueous sodium hydroxide (1.66 mL) for 2 days. The mixture was neutralized with acetic acid and concentrated. The residue was dissolved in dimethyl sulfoxide and purified by reverse-phase HPLC on a Gilson system (C18 column), eluting with 20-80% acetonitrile in water containing 0.1% trifluoroacetic acid, to give the title compound. MS (ESI) m/e 780.5 (M+H)⁺.

### 1.26.9 tert-butyl 6-(4-(benzo[d]thiazol-2-ylcarbamoyl)isoquinolin-6-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

To a mixture of Example 1.26.8 (122 mg), benzo[d]thiazol-2-amine (47.0 mg), 0-(7-azabenzotriazol-1-yl)-N,N,N′,N′-tetramethyluronium hexafluorophosphate (119 mg) in N,N-dimethylformamide (0.5 mL) was added N,N-diisopropylethylamine (273 μL). The mixture was stirred overnight and loaded onto an 80 g silica gel column, eluting with 5-100% heptanes in ethyl acetate to provide the title compound. MS (ESI) m/e 912.5 (M+H)⁺.

### 1.26.10 6-[4-(1,3-benzothiazol-2-ylcarbamoyl)isoquinolin-6-yl]-3-[1-((3,5-dimethyl-7-[2-(methylamino)ethoxy]tricyclo[3.3.1.13,7]dec-1-yl)methyl)-5-methyl-1H-pyrazol-4-yl]pyridine-2-carboxylic acid

Example 1.26.9 (100 mg) in dichloromethane (4 mL) was treated with trifluoroacetic acid (2 mL) for 3 hours and the mixture was concentrated. The residue was dissolved in dimethyl sulfoxide (5 mL) and purified by reverse-phase HPLC on a Gilson system (C18 column), eluting with 20-80/o acetonitrile in water containing 0.1° % trifluoroacetic acid, to give the title compound. ¹H NMR (400 MHz, dimethyl sulfoxide-d₆) δ ppm 13.27 (s, 1H), 9.58 (s, 1H), 9.03 (d, 2H), 8.53 (dd, 1H), 8.42 (d, 1H), 8.25 (t, 3H), 8.06 (d, 1H), 7.97 (d, 1H), 7.81 (d, 1H), 7.56-7.45 (m, 2H), 7.37 (t, 1H), 3.89 (s, 2H), 3.55 (t, 2H), 3.01 (t, 2H), 2.54 (t, 4H), 2.23 (s, 3H), 1.44 (s, 2H), 1.36-1.23 (m, 4H), 1.16 (s, 4H), 0.87 (s, 6H). MS (ESI) m/e 756.1 (M+H)⁺.

### 1.27 Synthesis of 6-[7-(1,3-benzothiazol-2-ylcarbamoyl)-1H-indol-2-yl]-3-[I-({3,5-dimethyl-7-[2-(methylamino)ethoxy]tricyclo[3.3.1.13,7]dec-1-yl}methyl)-5-methyl-1H-pyrazol-4-yl]pyridine-2-carboxylic acid (W3.27)

### 1.27.1 methyl 2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-1H-indole-7-carboxylate

To a stirred solution of methyl 2-(4.4,5,5-tetramethyl-1,3,2-dioxaborolan-2-yl)-1H-indole-7-carboxylate (370 mg), tris(dibenzylideneacetone)dipalladium(0) (30 mg), 1.2.3,4,5-pentaphenyl-1′-(di-tert-butylphosphino)ferrocene (30 mg) and potassium phosphate (550 mg) in tetrahydrofuran (2 mL) was added Example 1.1.11 (735 mg). The mixture was purged with nitrogen and stirred at 70° C. for 3 hours. The reaction was diluted with ethyl acetate and washed with water and brine. The aqueous layer was back extracted by ethyl acetate. The combined organic layers were dried over sodium sulfate, filtered and concentrated. The residue was purified via silica gel chromatography, eluting with 0-20% ethyl acetate in heptanes, to give the title compound. MS (ESI) m/e 780.4 (M−H)⁻.

### 1.27.2 2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-1H-indole-7-carboxylic acid

The title compound was prepared as described in Example 1.4.8, replacing Example 1.4.7 with Example 1.27.1. MS (ESI) m/e 766.4 (M−H)⁻.

### 1.27.3 tert-butyl 6-(7-(benzo[d]thiazol-2-ylcarbamoyl)-1H-indol-2-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

The title compound was prepared as described in Example 1.4.9, replacing Example 1.4.8 with Example 1.27.2. MS (ESI) m/e 898.4 (M−H)⁻.

### 1.27.4 6-[7-(1,3-benzothiazol-2-ylcarbamoyl)-1H-indol-2-yl]-3-[1-({3,5-dimethyl-7-[2-(methylamino)ethoxy]tricyclo[3.3.13,7]dec-1-yl}methyl)-5-methyl-1H-pyrazol-4-yl]pyridine-2-carboxylic acid

The title compound was prepared by substituting Example 1.27.3 for Example 1.1.13 in Example 1.1.14. ¹H NMR (501 MHz, dimethyl sulfoxide-d₆) δ ppm 13.01 (s, 1H), 11.19 (s, 1H), 8.27 (dd, 4H), 8.04 (d, 1H), 7.99 (d, 1H), 7.91 (d, 1H), 7.53-7.45 (m, 3H), 7.36 (t, 1H), 7.27 (t, 1H), 3.91 (s, 2H), 3.57 (t, 3H), 3.03 (t, 3H), 2.58-2.54 (m, 4H) 2.24 (s, 3H), 1.46 (s, 2H), 1.38-1.27 (m, 4H), 1.24-1.01 (m, 6H), 0.89 (s, 6H). MS (ESI) m/e 744.2 (M+H)⁺.

### 1.28 Synthesis of 3-(1-[3-(2-aminoethoxy)-5,7-dimethyltricyclo[3.3.1.13,7]dec-1-yl]methyl)-5-methyl-1H-pyrazol-4-yl)-6-[7-(1,3-benzothiazol-2-ylcarbamoyl)-1H-indol-2-yl]pyridine-2-carboxylic acid (W3.28)

### 1.28.1 methyl 2-[5-{1-[(3-{2-[bis(tert-butoxycarbonyl)amino]ethoxy}-5,7-dimethyltricyclo[3.3.1.13,7]dec-1-yl)methyl]-5-methyl-1H-pyrazol-4-yl}-6-(tert-butoxycarbonyl)pyridin-2-yl]-1H-indole-7-carboxylate

The title compound was prepared by substituting Example 1.23.3 for Example 1.1.11 in Example 1.27.1. MS (ESI) m/e 866.3 (M−H)⁻.

### 1.28.2 2-(6-(tert-butoxycarbonyl)-5-(1-((3-(2-((tert-butoxycarbonyl)amino)ethoxy)-5,7-dimethyladamantan-1-yl)methyl)-5-methyl-1H-pyrazol-4-yl)pyridin-2-yl)-1H-indole-7-carboxylic acid

The title compound was prepared as described in Example 1.4.8, replacing Example 1.4.7 with Example 1.28.1. MS (ESI) m/e 754.4 (M+H)⁺.

### 1.28.3 tert-butyl 6-(7-(benzo[d]thiazol-2-ylcarbamoyl)-1H-indol-2-yl)-3-(1-((3-(2-((tert-butoxycarbonyl)amino)ethoxy)-5,7-dimethyladamantan-1-yl)methyl)-5-methyl-1H-pyrazol-4-yl)picolinate

The title compound was prepared as described in Example 1.4.9, replacing Example 1.4.8 with Example 1.28.2. MS (ESI) m/e 886.5 (M+H)⁺.

### 1.28.4 3-(1-{[3-(2-aminoethoxy)-5,7-dimethyltricyclo[3.3.1.13,7]dec-1-yl]methyl}-5-methyl-1H-pyrazol-4-yl)-6-[7-(1,3-benzothiazol-2-ylcarbamoyl)-1H-indol-2-yl]pyridine-2-carboxylic acid

The title compound was prepared by substituting Example 1.28.3 for Example 1.1.13 in Example 1.1.14. ¹H NMR (501 MHz, dimethyl sulfoxide-d₆) δ ppm 13.00 (s, 1H), 11.19 (s, 1H), 8.29 (d, 1H), 8.23 (d, 1H), 8.03 (d, 1H), 7.98 (d, 1H), 7.90 (d, 1H), 7.80 (s, 1H), 7.63 (s, 3H), 7.50 (s, 1H), 7.49-7.44 (m, 2H), 7.39-7.32 (m, 1H), 7.25 (t, 1H), 3.90 (s, 2H), 2.90 (q, 2H), 2.23 (s, 3H), 1.45 (s, 2H), 1.31 (q, 4H), 1.23-1.00 (m, 7H), 0.88 (s, 6H). MS (ESI) m/e 730.2 (M+H)⁺.

### 1.29 Synthesis of 6-[7-(1,3-benzothiazol-2-ylcarbamoyl)-3-methyl-1H-indol-2-yl]-3-[l-({3,5-dimethyl-7-[2-(methylamino)ethoxy]tricyclo[3.3.1.13,7]dec-1-yl}methyl)-5-methyl-1H-pyrazol-4-yl]pyridine-2-carboxylic acid (W3.29) 1.29.1 methyl 3-methyl-1H-indole-7-carboxylate

To 7-bromo-3-methyl-1H-indole (1 g), dichloro[1,1′-bis(diphenylphosphino)ferrocene]palladium(II) dichloromethane adduct (0.070 g) in a 50 mL pressure bottle was added methanol (20 mL) and trimethylamine (1.327 mL). The reactor was purged with inert gas, followed by carbon monoxide. The reaction was heated to 100° C. for 20 hours at 60 psi. The solution was filtered and concentrated. The residue was purified by silica gel chromatography, eluting with a gradient of 5-30% ethyl acetate in heptanes, to give the title compound. MS (ESI) m/e 189.9 (M+H)⁺.

### 1.29.2 methyl 2-bromo-3-methyl-1H-indole-7-carboxylate

To a stirred suspension of Example 1.29.1 (70 mg) and 70 mg silica gel in dichloromethane (2 mL) was added 1-bromopyrrolidine-2,5-dione (70 mg). The mixture was protected from light by with aluminum foil and was stirred at room temperature under nitrogen for 30 minutes. The reaction mixture was filtered, washed with dichloromethane and purified via silica gel chromatography, eluting with 10-50% ethyl acetate in heptane, to provide the title compound. MS (ESI) m/e 267.6 (M+H)⁺.

### 1.293 methyl 3-methyl-2-(4,4,5,5-tetramethyl-1,3,2-dioxaborolan-2-yl)-1H-indole-7-carboxylate

To a stirred suspension of Example 1.29.2 (398 mg), 4,4,4′,4′,5,5,5′,5′-octamethyl-2,2′-bi(1,3,2-dioxaborolane) (1.2 g,) and potassium acetate (450 mg) in 1,4-dioxane (2 mL) was added bis(triphenylphosphine)palladium(II) dichloride (55 mg). The mixture was purged with nitrogen and heated at 115° C. under microwave conditions (Biotage Initiator) for 3 hours. The reaction was diluted with ethyl acetate and washed with water and brine. The aqueous layer was back extracted with ethyl acetate. The combined organic layer was dried over sodium sulfate, filtered and concentrated. The residue was purified via silica gel chromatography, eluting with 5-50% ethyl acetate in heptane, to give the title compound. MS (ESI) m/e 315.9 (M+H)⁺.

### 1.29.4 methyl 2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-3-methyl-1H-indole-7-carboxylate

Example 1.29.4 was prepared by substituting Example 1.29.3 for methyl 2-(4.4,5,5-tetramethyl-1,3,2-dioxaborolan-2-yl)-1H-indole-7-carboxylate in Example 1.27.1. MS (ESI) m/e 794.4 (M−H)⁻.

### 1.29.5 2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-3-methyl-1H-indole-7-carboxylic acid

Example 1.29.5 was prepared by substituting Example 1.29.4 for Example 1.4.7 in Example 1.4.8. MS (ESI) m/e 780.4 (M−H)⁻.

### 1.29.6 tert-butyl 6-(7-(benzo[d]thiazol-2-ylcarbamoyl)-3-methyl-1H-indol-2-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

Example 1.29.6 was prepared by substituting Example 1.29.5 for Example 1.4.8 in Example 1.4.9. MS (ESI) m/e 912.4 (M−H)⁻.

### 1.29.7 6-[7-(1,3-benzothiazol-2-ylcarbamoyl)-3-methyl-1H-indol-2-yl]-3-[1-({3,5-dimethyl-7-[2-(methylamino)ethoxy]tricyclo[3.3.1.13,7]dec-1-yl}methyl)-5-methyl-1H-pyrazol-4-yl]pyridine-2-carboxylic acid

The title compound was prepared by substituting Example 1.29.6 for Example 1.1.13 in Example 1.1.14. ¹H NMR (501 MHz, dimethyl sulfoxide-d₆) δ ppm 12.97 (s, 1H), 11.04 (s, 1), 8.34-8.23 (m, 3H), 8.06 (d, 1H), 8.02 (dd, 2H), 7.93 (d, 1H), 7.79 (d, 1H), 7.51 (s, 1H), 7.48 (ddd, 1H), 7.38-7.32 (m, 1H), 7.25 (t, 1H), 3.91 (s, 2H), 3.56 (t, 2H), 3.03 (p, 2H), 2.67 (s, 3H), 2.56 (t, 3H), 2.25 (s, 3H), 1.46 (s, 2H), 1.38-1.26 (m, 4H), 1.24-1.13 (m, 4H), 1.06 (q, 2H), 0.89 (s, 6H). MS (ESI) m/e 758.2 (M+H)⁺.

### 1.30 Synthesis of 6-[8-(1,3-benzothiazol-2-ylcarbamoyl)-3,4-dihydroisoquinolin-2(1H)-yl]-3-(1-{[3,5-dimethyl-7-(2-{[-(methylsulfonyl)piperidin-4-yl]amino}ethoxy)tricyclo[3.3.1.13,7]dec-1-yl]methyl}-5-methyl-1H-pyrazol-4-yl)pyridine-2-carboxylic acid (W3.30)

### 1.30.1 tert-butyl 6-(8-(benzo[d]thiazol-2-ylcarbamoyl)-3,4-dihydroisoquinolin-2(1H)-yl)-3-(1-(((1r,7r)-3,5-dimethyl-7-(2-((1-(methylsulfonyl)piperidin-4-yl)amino)ethoxy)adamantan-1-yl)methyl)-5-methyl-1H-pyrazol-4-yl)picolinate

A solution of Example 1.18.18 (0.060 g), 1-(methylsulfonyl)piperidin-4-one (0.015 g) and sodium triacetoxyborohydride (0.024 g) was stirred in dichloromethane (0.5 mL) at room temperature. After 30 minutes, the reaction mixture was concentrated. The crude material was dissolved in N,N-dimethylformamide (1.5 mL) and water (0.5 mL) and purified by preparatory reverse-phase HPLC on a Gilson 2020 system using a gradient of 5% to 85% acetonitrile/water. The product-containing fractions were lyophilized to give the title compound as a trifluoroacetic acid salt. MS (ESI) m/e 963.9 (M+H)⁺.

### 1.30.2 6-[8-(1,3-benzothiazol-2-ylcarbamoyl)-3,4-dihydroisoquinolin-2(1H)-yl]-3-(1-{[3,5-dimethyl-7-(2-({[1-(methylsulfonyl)piperidin-4-yl]amino}ethoxy)tricyclo[3.3.1.13,7]dec-1-yl]methyl}-5-methyl-1H-pyrazol-4-yl)pyridine-2-carboxylic acid

A solution of Example 1.30.1 (0.060 g) was dissolved in dichloromethane (0.5 mL) and treated with trifluoroacetic acid (0.5 mL) overnight. The reaction mixture was concentrated. The residue was dissolved in N,N-dimethylformamide (1.5 mL) and water (0.5 mL) and was purified by preparatory reverse-phase HPLC on a Gilson 2020 system using a gradient of 5% to 85% acetonitrile/water. The product-containing fractions were lyophilized to give the title compound. ¹H NMR (400 MHz, dimethyl sulfoxide-d₆) δ ppm 12.90 (s, 1H), 8.53 (d, 2H), 8.08 (d, 1H), 7.84 (d, 1H), 7.66 (d, 1H), 7.58-7.45 (m, 4H), 7.41 (td, 2H), 7.33 (s, 1H), 7.00 (d, 1H), 5.00 (s, 2H), 3.93 (s, 2H), 3.88 (s, 2H), 3.62 (d, 4H), 3.22 (h, 2H), 3.12, 3.06 (s, 2H), 2.93 (s, 3H), 2.79 (d, 2H), 2.15 (s, 3H), 2.11 (s, 1H), 1.61 (qd, 2H), 1.48 (s, 2H), 1.37 (s, 2H), 1.19 (s, 4H), 1.10 (s, 2H), 0.91 (s, 8H). MS (ESI) m/e 907.2 (M+H)⁺.

### 1.31 Synthesis of 6-[8-(1,3-benzothiazol-2-ylcarbamoyl)-3,4-dihydroisoquinolin-2(1H)-yl]-3-(1-{([3,5-dimethyl-7-(2-{[1-(methylsulfonyl)azetidin-3-yl]amino}ethoxy)tricyclo[3.3.1.13,7]dec-1-yl]methyl}-5-methyl-1H-pyrazol-4-yl)pyridine-2-carboxylic acid (W3.31)

A solution of Example 1.18.18 (0.050 g), 1-(methylsulfonyl)azetidin-3-one (0.014 g) and sodium triacetoxyborohydride (0.020 g) was stirred in dichloromethane (0.50 mL) at room temperature. After 30 minutes, acetic acid (5.35 μL) was added and stirring was continued at room temperature overnight. Trifluoroacetic acid (0.5 mL) was added to the reaction and was stirring continued overnight. The reaction mixture was concentrated. The residue was dissolved in a mixture of N,N-dimethylformamide (2 mL) and water (0.5 mL) and was purified by preparatory reverse-phase HPLC on a Gilson 2020 system using a gradient of 5% to 70% acetonitrile/water. The product-containing fractions were lyophilized to give the title compound. ¹H NMR (400 MHz, dimethyl sulfoxide-d₆) δ ppm 12.86 (s, 1H), 9.13 (s, 2H), 8.03 (d, 1H), 7.79 (d, 1H), 7.62 (d, 1H), 7.54-7.41 (m, 3H), 7.36 (td, 2H), 7.29 (s, 1H), 6.96 (d, 1H), 4.96 (s, 2H), 4.09 (s, 2H), 4.08 (s, 1H), 3.98 (s, 2H), 3.89 (s, 2H), 3.84 (s, 2H), 3.56 (s, 2H), 3.05 (s, 3H), 3.03 (s, 2H), 3.02 (s, 1H), 2.11 (s, 2H), 1.44 (s, 2H), 1.31 (q, 4H), 1.14 (s, 4H), 1.06 (s, 2H), 0.87 (s, 6H). MS (ESI) m/e 879.7 (M+H)⁺.

### 1.32 Synthesis of 3-{1-[(3-(2-[(3-amino-3-oxopropyl)amino]ethoxy}-5,7-dimethyltricyclo[3.3.1.13,7]dec-1-yl)methyl]-5-methyl-1H-pyrazol-4-yl)-6-[8-(1,3-benzothiazol-2-ylcarbamoyl)-3,4-dihydroisoquinolin-2(1H)-yl]pyridine-2-carboxylic acid (W3.32)

### 1.32.1 tert-butyl 3-(1-((3-(2-((3-amino-3-oxopropyl)amino)ethoxy)-5,7-dimethyladamantan-1-yl)methyl)-5-methyl-1H-pyrazol-4-yl)-6-(8-(benzo[d]thiazol-2-ylcarbamol)-3,4-dihydroisoquinolin-2(1H)-yl)picolinate

A mixture of Example 1.18.18 (245 mg) and acrylamide (217 mg) in N,N-dimethylformnnamide (5 mL) was heated at 50° C. for 3 days and was purified by reverse phase HPLC, eluted with 30%-80% acetonitrile in 0.1% trifluoroacetic acid in water solution, to provide the title compound. ¹H NMR (400 MHz, dimethyl sulfoxide-d₆) δ ppm 12.83 (s, 1H), 8.30 (s, 2H), 8.00 (dd, 1H), 7.76 (d, 1H), 7.57 (d, 2H), 7.44 (ddd, 3H), 7.39-7.29 (m, 2H), 7.21 (s, 1H), 7.13 (s, 1H), 6.91 (d, 1H), 4.95 (s, 2H), 3.81 (d, 4H), 3.53 (t, 2H), 3.05 (dq, 6H), 2.06 (s, 3H), 1.43 (s, 2H), 1.27 (q, 4H), 1.13 (d, 15H), 0.82 (s, 6H). MS (ESI) m e 873.8 (M+H)⁺.

### 1.32.2 3-{1-[(3-(2-[(3-amino-3-oxopropyl)amino]ethoxy)-5,7-dimethyltricyclo[3.3.1.13,7]dec-1-yl)methyl]-5-methyl-1H-pyrazol-4-yl}-6-[8-(1,3-benzothiazol-2-ylcarbamoyl)-3,4-dihydroisoquinolin-2(1H)-yl]pyridine-2-carboxylic acid

The title compound was prepared using the procedure in Example 1.26.10, replacing Example 1.26.9 with Example 1.32.1. ¹H NMR (400 MHz, dimethyl sulfoxide-d₆) δ ppm 8.29 (s, 2H), 8.00 (dd, 1H), 7.76 (d, 1H), 7.63-7.52 (m, 2H), 7.49-7.38 (m, 3H), 7.37-7.29 (m, 2H), 7.25 (s, 1H), 7.11 (s, 1H), 6.92 (d, 1H), 4.92 (s, 2H), 3.53 (t, 2H), 3.04 (ddt, 6H), 2.07 (s, 3H), 1.39 (s, 2H), 1.26 (q, 4H), 1.16-0.93 (m, 6H), 0.83 (s, 6H). MS (ESI) m/e 817.2 (M+H)⁺.

### 1.33 Synthesis of 6-[3-(1,3-benzothiazol-2-ylcarbamoyl)-1H-indazol-5-yl]-3-[1-({3,5-dimethyl-7-[2-(methylamino)ethoxy]tricyclo[3.3.1.13,7]dec-1-yl}methyl)-5-methyl-1H-pyrazol-4-yl]pyridine-2-carboxylic acid (W3.33)

### 1.33.1 5-(4,4,5,5-tetramethyl-[1,3,2 ]dioxaborolan-2-yl)-1-(2-trimethylsilanyl-ethoxymethyl)-1H-indazole-3-carboxylic acid ethyl ester

Ethyl 5-(4.4,5,5-tetramethyl-1,3,2-dioxaborolan-2-yl)-1H-indazole-3-carboxylate (1000 mg) was dissolved in N,N-dimethylformamide (30 mL). Sodium hydride (60% in mineral oil, 83 mg) was added, and the solution and was stirred at room temperature for 20 minutes. (2-(Chloromethoxy)ethyl)trimethylsilane (580 mg) was added, and the solution was stirred at room temperature for 90 minutes. The reaction was quenched with saturated aqueous ammonium chloride (10 mL) and diluted with water (90 mL). The solution was extracted with 70% ethyl acetate in heptanes (50 mL) twice. The combined organic portions were washed with water (25 mL) and then brine (25 mL). The solution was dried on anhydrous sodium sulfate, filtered and concentrated under reduced pressure. The residue was purified by flash column chromatography on silica gel, eluting with 10-30% ethyl acetate in heptanes. The solvent was removed under reduced pressure to yield the title compound. MS (ESI) m/e 447 (M+H)⁺.

### 1.33.2 ethyl 5-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-1-((2-(trimethylsilyl)ethoxy)methyl)-1H-indazole-3-carboxylate

Example 1.33.1 (335 mg) and Example 1.1.11 (483 mg) were dissolved in 1,4-dioxane (3 mL). 2 M aqueous sodium carbonate (1.13 mL) was added, and the solution was degassed and flushed with nitrogen three times. Dichloro[1,1′-bis(diphenylphosphino)ferrocene]palladium(II) (61 mg) was added, and the solution was degassed and flushed with nitrogen once. The solution was heated at 75° C. for 16 hours. The solution was cooled, and 0.1 M aqueous HCl (25 mL) was added. The solution was extracted with ethyl acetate (50 mL) twice. The combined organic portions were washed with brine (25 mL) and dried on anhydrous sodium sulfate. The solution was filtered, concentrated under reduced pressure and purified by flash column chromatography on silica gel, eluting with 50% ethyl acetate in heptanes. The solvent was removed under reduced pressure to yield the title compound. MS (ESI) m/e 927 (M+NH₄—H₂O)⁺.

### 1.33.3 5-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-1-((2-(trimethylsilyl)ethoxy)methyl)-1H-indazole-3-carboxylic acid

The title compound was prepared by substituting Example 1.33.2 for Example 1.13.9 in Example 1.13.10. MS (ESI) m/e 899 (M+H)⁺, 897 (M−H)⁻.

### 1.33.4 tert-butyl 6-(3-(benzo[d]thiazol-2-ylcarbamoyl)-1-((2-(trimethylsilyl)ethoxy)methyl)-1H-indazol-5-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

The title compound was prepared by substituting Example 1.33.3 for Example 1.13.10 in Example 1.13.11. MS (ESI) m/e 1030 (M+NH₄—H₂O)⁺, 1029 (M−H)⁻.

### 1.33.5 6-[3-(1,3-benzothiazol-2-ylcarbamoyl)-1H-indazol-5-yl]-3-[1-({3,5-dimethyl-7-[2-(methylamino)ethoxy]tricyclo[3.3.1.13,7]dec-1-yl}methyl)-5-methyl-1H-pyrazol-4-yl]pyridine-2-carboxylic acid

Example 1.33.4 (83 mg) was dissolved in dichloromethane (0.5 mL). Trifluoroacetic acid (740 mg) was added, and the solution was stirred at room temperature for 16 hours. The solvents were removed under reduced pressure. The residue was dissolved in 1,4-dioxane (1 mL), and 1 M aqueous sodium hydroxide (0.5 mL) was added. The solution was stirred at room temperature for 60 minutes. The reaction was quenched with trifluoroacetic acid (0.1 mL) and purified by reverse-phase HPLC using 10-85% acetonitrile in water (w/0.1% trifluoroacetic acid) over 30 minutes on a Grace Reveleris® equipped with a Phenomenex® Luna® column: C18(2), 100 A, 150×30 mm. Product fractions were combined, frozen, and lyophilized to yield the title compound as the bis trifluoroacetic acid salt. ¹H NMR (400 MHz, dimethyl sulfoxide-d₆) δ ppm 14.23 (s, 1H), 12.58 (bs, 1H), 8.97 (s, 1H), 8.34-8.29 (m, 3H), 8.22 (d, 1H), 8.04 (d, 1H), 7.91 (d, 1H), 7.87-7.81 (m, 2H), 7.51-7.45 (m, 2H), 7.36 (t, 1H), 3.92 (s, 3H), 3.58 (m, 2H), 3.04 (m, 2H), 2.58-2.56 (m, 2H), 2.26 (s, 3H), 1.47 (s, 2H), 1.34 (q, 4H), 1.22-1.14 (m, 4H), 1.07 (q, 2H), 0.89 (m, 6H). MS (ESI) m/e 745 (M+H)⁺, 743 (M−H)⁻.

### 1.34 Synthesis of 6-[3-(1,3-benzothiazol-2-ylcarbamoyl)-1H-indol-5-yl]-3-[1-({3,5-dimethyl-7-[2-(methylamino)ethoxy]tricyclo[3.3.1.13,7]dec-1-yl}methyl)-5-methyl-1H-pyrazol-4-yl]pyridine-2-carboxylic acid (W3.34)

### 1.34.1 5-(4,4,5,5-tetramethyl-[1,3,2]dioxaborolan-2-yl)-1-(2-trimethylsilanyl-ethoxymethyl)-1H-indole-3-carboxylic acid methyl ester

The title compound was prepared by substituting methyl 5-(4.4,5,5-tetramethyl-1,3,2-dioxaborolan-2-yl)-1H-indole-3-carboxylate for ethyl 5-(4,4,5,5-tetramethyl-1,3,2-dioxaborolan-2-yl)-1H-indazole-3-carboxylate in Example 1.33.1. MS (ESI) m/e 432 (M+H)⁺.

### 1.34.2 methyl 5-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-1-((2-(trimethylsilyl)ethoxy)methyl)-1H-indole-3-carboxylate

The title compound was prepared by substituting Example 1.34.1 for Example 1.33.1 in Example 1.33.2. MS (ESI) m/e 912 (M+H)⁺.

### 1.34.3 5-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-1-((2-(trimethylsilyl)ethoxy)methyl)-1H-indole-3-carboxylic acid

The title compound was prepared by substituting Example 1.34.2 for Example 1.13.9 in Example 1.13.10. MS (ESI) m/e 898 (M+H)⁺, 8% (M−H)⁻.

### 1.34.4 tert-butyl 6-(3-(benzo[d]thiazol-2-ylcarbamoyl)-1-((2-(trimethylsilyl)ethoxy)methyl)-1H-indol-5-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

The title compound was prepared by substituting Example 1.34.3 for Example 1.13.10 in Example 1.13.11. MS (ESI) m/e 1030 (M+H)⁺, 1028 (M−H)⁻,

### 1.34.5 6-[3-(1,3-benzothiazol-2-ylcarbamoyl)-1H-indol-5-yl]-3-[1-((3,5-dimethyl-7-[2-(methylamino)ethoxy]tricyclo[3.3.1.13,7]dec-1-yl)methyl)-5-methyl-1H-pyrazol-4-yl]pyridine-2-carboxylic acid

The title compound was prepared by substituting Example 1.34.4 for Example 1.33.4 in Example 1.33.5. ¹H NMR (400 MHz, dimethyl sulfoxide-d₆) δ ppm 12.47 (bs, 1H), 12.18 (s, 1H), 9.01 (s, 1H), 8.70 (d, 1H), 8.28 (bs, 3H), 8.12 (d, 1H), 8.05 (dd, 1H), 7.99 (d, 1H), 7.86 (d, 1H), 7.76 (d, 1H), 7.64 (d, 1H), 7.50 (s, 1H), 7.46 (td, 1H), 7.32 (t, 1H), 3.92 (s, 3H), 3.58 (m, 2H), 3.04 (m, 2H), 2.57 (m, 2H), 2.26 (s, 3H), 1.47 (s, 2H), 1.34 (q, 4H), 1.24-1.14 (m, 4H), 1.08 (m, 2H), 0.90 (s, 61f). MS (ESI) m/e 744 (M+H)⁺, 742 (M−H)⁻.

### 1.35 Synthesis of 6-[3-(1,3-benzothiazol-2-ylcarbamoyl)-1H-pyrrolo[2,3-b]pyridin-5-yl]-3-[1-({3,5-dimethyl-7-[2-(methylamino)ethoxy]tricyclo[3.3.1.13,7]dec-1-yl}methyl)-5-methyl-1H-pyrazol-4-yl]pyridine-2-carboxylic acid (W3.35)

### 1.35.1 5-bromo-1-(2-trimethylsilanyl-ethoxymethyl)-1H-pyrrolo[2,3-b]pyridine-3-carboxylic acid methyl ester

The title compound was prepared by substituting methyl 5-bromo-1H-pyrrolo[2,3-b]pyridine-3-carboxylate for ethyl 5-(4,4,5,5-tetramethyl-1,3,2-dioxaborolan-2-yl)-1H-indazole-3-carboxylate in Example 1.33.1. MS (ESI) m/e 385, 387 (M+H)⁺.

### 1.35.2 5-(4,4,5,5-tetramethyl-[1,3,2]dioxaborolan-2-yl)-1-(2-trimethylsilanyl-ethoxymethyl)-1H-pyrrolo[2,3-b]pyridine-3-carboxylic acid methyl ester

The title compound was prepared by substituting Example 1.35.1 for Example 1.13.7 in Example 1.13.8. MS (ESI) m/e 433(M+H)⁺.

### 1.35.3 methyl 5-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-1-((2-(trimethylsilyl)ethoxy)methyl)-1H-pyrrolo[2,3-h]pyridine-3-carboxylate

The title compound was prepared by substituting Example 1.35.2 for Example 1.33.1 in Example 1.33.2. MS (ESI) m/e 913 (M+H)⁺.

### 1.35.4 5-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-1-((2-(trimethylsilyl)ethoxy)methyl)-1H-pyrrolo[2,3-h]pyridine-3-carboxylic acid

The title compound was prepared by substituting Example 1.35.3 for Example 1.13.9 in Example 1.13.10. MS (ESI) m/e 899 (M+H)⁺, 897 (M−H)⁻.

### 1.35.5 tert-butyl 6-(3-(benzo[d]thiazol-2-ylcarbamoyl)-1-((2-(trimethylsilyl)ethoxy)methyl)-1H-pyrrolo[2,3-b]pyridin-5-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

The title compound was prepared by substituting Example 1.35.4 for Example 1.13.10 in Example 1.13.11. MS (ESI) m e 1031 (M+H)⁺, 1029 (M−H)⁻.

### 1.35.6 6-[3-(1,3-benzothiazol-2-ylcarbamoyl)-1H-pyrrolo[2,3-b]pyridin-5-yl]-3-[1-((3,5-dimethyl-7-[2-(methylamino)ethoxy]tricyclo[3.3.1.13,7]dec-1-yl)methyl)-5-methyl-1H-pyrazol-4-yl]pyridine-2-carboxylic acid

The title compound was prepared by substituting Example 1.35.5 for Example 1.33.4 in Example 1.33.5. ¹H NMR (400 MHz, dimethyl sulfoxide-4) δ ppm 12.74 (d, 1H), 12.62 (bs, 1H), 9.26 (d, 1H), 9.13 (d, 1H), 8.83 (d, 1H), 8.28 (bs, 2H), 8.25 (d, 1H), 7.99 (d, 1H), 7.91 (d, 1H), 7.78 (d, 1H), 7.51 (s, 1H), 7.47 (t, 1H), 7.33 (t, 1H), 3.92 (s, 3H), 3.58 (t, 2H), 3.04 (m, 2H), 2.57 (t, 2H), 2.26 (s, 3H), 1.47 (s, 2H), 1.34 (q, 4H), 1.20 (t 4H), 1.08 (q, 2H), 0.90 (s, 6H). MS (ESI) m/e 745 (M+H)⁺, 743 (M−H)⁻.

### 1.36 Synthesis of 6-(8-(benzo[d]thiazol-2-ylcarbamoyl)-3,4-dihydroisoquinolin-2(1H)-yl)-3-(1-((3-(2-((2-(N,N-dimethylsulfamoyl)ethyl)amino)ethoxy)-5,7-dimethyladamantan-1-yl)methyl)-5-methyl-1H-pyrazol-4-yl)picolinic acid (W3.36)

To a solution of Example 1.18.18 (69.8 mg) in N,N-dimethylformamide (6 mL) was added N,N-dimethylethenesulfonamide (118 mg), N,N-diisopropylethylamine (0.2 mL) and H₂O (0.2 mL). The mixture was stirred at room temperature 4 days. The reaction mixture was diluted with ethyl acetate (200 mL), washed with water and brine, and dried over anhydrous sodium sulfate. After evaporation of the solvent, the residue was dissolved in dichloromethane and trifluoroacetic acid (10 mL, 1:1), and the resulting solution was stirred overnight. The solvents were removed under reduced pressure. The residue was diluted with N,N-dimethylformamide (2 mL), filtered and purified by reverse-phase HPLC on a Gilson system (C18 column), eluting with 20-80% acetonitrile in water containing 0.1% trifluoroacetic acid, to give the title compound. ¹H NMR (400 MHz, dimethyl sulfoxide-d₆) δ ppm 12.82 (s, 1H), 8.53 (s, 2H), 8.00 (dd, 1H), 7.76 (d, 1H), 7.59 (dd, 1H), 7.53-7.37 (m, 4H), 7.37-7.28 (m, 2H), 7.26 (s, 1H), 6.92 (d, 1H), 4.92 (s, 2H), 3.80 (s, 2H), 3.54 (t, 2H), 3.44-3.34 (m, 2H), 3.30 (s, 2H), 3.11 (s, 2H), 2.98 (t, 2H), 2.77 (s, 6H), 2.07 (s, 3H), 1.39 (s, 2H), 1.27 (q, 4H), 1.11 (s, 4H), 1.06-0.93 (m, 2H), 0.83 (s, 7H). MS (ESI) m/e 881.2 (M+H)⁺.

### 1.37 Synthesis of 6-[8-(1,3-benzothiazol-2-ylcarbamoyl)naphthalen-2-yl]-3-{1-[(3-{2-[(3-hydroxypropyl)amino]ethoxy}-5,7-dimethyltricyclo[3.3.1.13,7]dec-1-yl)methyl]-5-methyl-1H-pyrazol-4-yl}pyridine-2-carboxylic acid (W3.37)

### 1.37.1 2-((3,5-dimethyl-7-((5-methyl-4-(4,4,5,5-tetramethyl-1,3,2-dioxaborolan-2-yl)-1H-pyrazol-1-yl)methyl)adamantan-1-yl)oxy)ethanol

To a solution of Example 1.1.6 (8.9 g) and 11,1′-bis(diphenylphosphino)ferroceneldichloropalladium(II) dichloromethane (818 mg) in acetonitrile (120 mL) was added triethylamine (10 mL) and pinacolborane (12.8 mL). The mixture was stirred at reflux overnight. The mixture was cooled to room temperature and used in the next reaction directly. MS (ESI) m/e 467.3 (M+Na)⁺.

### 1.37.2 tert-butyl 6-chloro-3-(1-((3-(2-hydroxyethoxy)-5,7-dimethyladamantan-1-yl)methyl)-5-methyl-1H-pyrazol-4-yl)picolinate

To a solution of tert-butyl 3-bromo-6-chloropicolinate (6.52 g) in tetrahydrofuran (100 mL) and water (20 mL) was added Example 1.37.1 (9.90 g), (1S,3R,5R,7S)-1.3,5,7-tetramethyl-8-tetradecyl-2,4,6-trioxa-8-phosphaadamantane (0.732 g), tris(dibenzylideneacetone)dipalladium(0) (1.02 g), and potassium phosphate (23.64 g), and the mixture was stirred at reflux overnight. The solvents were removed under vacuum. The residue was dissolved in ethyl acetate (500 mL), washed with water and brine, and dried over anhydrous sodium sulfate. Filtration and evaporation of the solvent gave a residue that purified by silica gel chromatography, eluting with 20% ethyl acetate in heptane, to give the title compound. MS (ESI) m/e 530.3 (M+H)⁺.

### 1.37.3 tert-butyl 3-{1-[(3-{2-[bis(tert-butoxycarbonyl)amino]ethoxy}-5,7-dimethyltricyclo[3.3.1.13,7]decan-1-yl)methyl]-5-methyl-1H-pyrazol-4-yl}-6-chloropyridine-2-carboxylatetert-butyl 6-chloro-3-(l-((3,5-dimethyl-7-(2-((methylsulfonyl)oxy)ethoxy)adamantan-1-yl)methyl)-5-methyl-1H-pyrazol-4-yl)picolinate

To a cooled (0° C.) stirring solution of Example 1.37.2 (3.88 g) in dichloromethane (30 mL) and triethylamine (6 mL) was added methanesulfonyl chloride (2.52 g). The mixture was stirred at room temperature for 4 hours. The reaction mixture was diluted with ethyl acetate (400 mL), washed with water and brine, and dried over anhydrous sodium sulfate. Filtration and evaporation of the solvent gave the title compound, which was used in the next reaction without further purification. MS (ESI) m/e 608.1 (M+H)⁺.

### 1.37.4 tert-butyl 3-{1-[(3-(2-[bis(tert-butoxycarbonyl)amino]ethoxy)-5,7-dimethyltricyclo[3.3.1.13,7]dec-1-yl)methyl]-5-methyl-1H-pyrazol-4-yl}-6-chloropyridine-2-carboxylate

To a solution of Example 1.37.3 (151 mg) in N,N-dimethylformamide (3 mL) was added di-t-butyl iminodicarboxylate (54 mg). The mixture was stirred at room temperature overnight. The reaction mixture was diluted with ethyl acetate (200 mL), washed with water and brine, and dried over anhydrous sodium sulfate. Filtration and evaporation of the solvent gave the title compound, which was used in the next step without further purification. MS (ESI) m/e 729.4 (M+H)⁺.

### 1.37.5 7-(6-(tert-butoxycarbonyl)-5-(1-((3-(2-((tert-butoxycarbonyl)amino)ethoxy)-5,7-dimethyladamantan-1-yl)methyl)-5-methyl-1H-pyrazol-4-yl)pyridin-2-yl)-1-naphthoic acid

To a solution of methyl 7-(4,4,5,5-tetramethyl-1,3,2-dioxaborolan-2-yl)-1-naphthoate (257 mg) in 1,4-dioxane (10 mL) and water (5 mL) was added Example 1.37.4 (600 mg), bis(triphenylphosphine)palladium(II) dichloride (57.8 mg), and cesium fluoride (375 mg). The mixture was stirred at 120° C. for 30 minutes under microwave conditions (Biotage Initiator). The mixture was diluted with ethyl acetate (200 mL), washed with water and brine, dried over anhydrous sodium sulfate, filtered and concentrated. Evaporation of the solvent gave a residue that purified by silica gel chromatography, eluting with 20% ethyl acetate in heptane, to give an intermediate di-ester. The residue was dissolved in tetrahydrofuran (10 mL), methanol (5 mL) and water (5 mL) and LiOH H₂O (500 mg) was added. The mixture was stirred at room temperature overnight. The mixture was acidified with aqueous 2N HCl, dissolved in 400 mL of ethyl acetate, washed with water and brine and dried over anhydrous sodium sulfate. Filtration and evaporation of the solvent gave the title compound. MS (APCI) m/e 765.3 (M+H)⁺.

### 1.37.6 3-(l-((3-(2-aminoethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic acid

To a solution of Example 1.37.5 (500 mg) in dichloromethane (10 mL) was added benzo[d]thiazol-2-amine (98 mg), 1-ethyl-3-[3-(dimethylamino)propy l]-carbodiimide hydrochloride (251 mg) and 4-(dimethylamino)pyridine (160 mg). The mixture was stirred at room temperature overnight. The reaction mixture was diluted with ethyl acetate (400 mL), washed with water and brine, dried over anhydrous sodium sulfate, filtered and concentrated. The residue was dissolved in dichloromethane and trifluoroacetic acid (10 mL, 1:1), and the solution was stirred overnight. The solvents were removed, and the residue was dissolved in N,N-dimethylformamide (12 mL) and purified by reverse-phase HPLC on a Gilson system (C18 column), eluting with 20-80% acetonitrile in water containing 0.1% trifluoroacetic acid, to give the title compound. MS (ESI) m/e 741.2 (M+H)⁺.

### 1.37.7 3-((tert-butyldimethylsilyl)oxy)propanal

To a solution of dimethyl sulfoxide (2.5 mL) in dichloromethane (40 mL) at −78° C. was added oxalyl chloride (1.5 mL). The mixture was stirred 20 minutes at −78° C., and a solution of (3-((tert-butyldimethylsilyl)oxy)propan-1-ol (1.9 g) in dichloromethane (10 mL) was added by syringe. After 1 hour, triethylamine (5 mL) was added. The cooling bath was removed, and the reaction was stirred overnight. The reaction mixture was diluted with ethyl acetate (300 mL), washed with water and brine, and dried over anhydrous sodium sulfate. Filtration and evaporation of solvent gave the title compound. MS (DCI) m/e 206.0(M+NH₄)⁺.

### 1.37.8 6-[8-(1,3-benzothiazol-2-ylcarbamoyl)naphthalen-2-yl]-3-{1-[(3-{2-[(3-hydroxypropyl)amino]ethoxy}-5,7-dimethyltricyclo[3.3.1.13,7]dec-1-yl)methyl]-5-methyl-1H-pyrazol-4-yl}pyridine-2-carboxylic acid

To a solution of Example 1.37.6 (125 mg) in dichloromethane (10 mL) was added Example 1.37.7 (32 mg). The mixture was stirred at room temperature for 1 hour, and NaBH(OAc)₃ (107 mg) was added to the reaction mixture. The mixture was stirred at room temperature overnight. To the reaction mixture was added 2N aqueous sodium hydroxide (5 mL), and the reaction stirred for 4 hours. The mixture was neutralized with aqueous 2N HCl and extracted with ethyl acetate (100 mL×3). The combined organic layers were washed with aqueous 2% HCl, water and brine and dried over anhydrous sodium sulfate. Filtration and evaporation of the solvent gave a residue that was purified by reverse-phase HPLC on a Gilson system (C18 column), eluting with 20-80% acetonitrile in water containing 0.1% trifluoroacetic acid, to give a solid. The residue was dissolved in tetrahydrofuran (6 mL) and tetrabutyl ammonium fluoride (1 M in tetrahydrofuran, 4 mL) was added. The mixture was stirred at room temperature for 2 hours, and the solvents were removed under vacuum. The residue was dissolved in dimethyl sulfoxide/methanol (1:1, 12 mL) and was purified by reverse-phase HPLC on a Gilson system (C18 column), eluting with 20-80% acetonitrile in water containing 0.1% trifluoroacetic acid, to give the title compound. ¹H NMR (501 MHz, dimethyl sulfoxide-d₆) δ ppm 13.09 (s, 1H), 9.01 (s, 1H), 8.36 (dd, 1H), 8.20 (ddd, 5H), 8.09-8.02 (m, 1H), 8.03-7.95 (m, 1H), 7.92 (d, 1H), 7.80 (d, 1H), 7.69 (dd, 1H), 7.53-7.43 (m, 2H1), 7.36 (ddd, 1H), 3.89 (s, 2H), 3.56 (t, 2H), 3.47 (t, 2H), 3.10-2.93 (m, 4H), 2.22 (s, 3H), 1.78-1.68 (m, 2H), 1.44 (s, 2H), 1.30 (q, 4H), 1.20-1.11 (m, 4H), 1.04 (q, 2H), 0.87 (s, 7H). MS (ESI) m/e 799.2 (M+H)⁺.

### 1.38 Synthesis of 6-[8-(1,3-benzothiazol-2-ylcarbamoyl)-3,4-dihydroisoquinolin-2(1H)-yl]-3-(1-{[3-(2-{[3-(dimethylamino)-3-oxopropyl]amino}ethoxy)-5,7-dimethyltricyclo[3.3.1.13,7]dec-1-yl]methyl}-5-methyl-1H-pyrazol-4-yl)pyridine-2-carboxylic acid (W3.38)

To a solution of Example 1.18.18 (55 mg) in N,N-dimethylformamide (6 mL) was added N,N-dimethylacrylamide (73.4 mg), N,N-diisopropylethylamine (0.2 mL) and water (0.2 mL). The mixture was stirred at room temperature 4 days. The reaction mixture was diluted with ethyl acetate (200 mL), washed with water and brine, and dried over anhydrous sodium sulfate. After filtration and evaporation of the solvent, the residue was dissolved in dichloromethane and trifluoroacetic acid (10 mL, 1:1). After stirring for 16 hours, the mixture was concentrated under reduced pressure. The residue was dissolved in N,N-dimethylformamide (8 mL) and purified by reverse-phase HPLC on a Gilson system (C18 column), eluting with 20-80% acetonitrile in water containing 0.1% trifluoroacetic acid, to give the title compound. ¹H NMR (400 MHz, dimethyl sulfoxide-d₆) δ ppm 12.84 (s, 1H), 8.22 (s, 3H), 8.02 (d, 1H), 7.78 (d, 1H), 7.60 (d, 1H), 7.55-7.39 (m, 3H), 7.39-7.30 (m, 2H), 7.27 (s, 1H), 6.94 (d, 1H), 4.94 (s, 2H), 3.87 (t, 2H), 3.81 (s, 2H), 3.55 (t, 2H), 3.20-2.95 (m, 6H), 2.92 (s, 3H), 2.82 (s, 3H), 2.69 (q, 3H), 2.09 (s, 3H), 1.40 (s, 2H), 1.28 (q, 4H), 1.14 (d, 4H), 1.07-0.94 (m, 2H), 0.85 (s, 8H). MS (ESI) m/e 845.3 (M+H)⁺.

### 1.39 Synthesis of 6-[8-(1,3-benzothiazol-2-ylcarbamoyl)-3,4-dihydroisoquinolin-2(1H)-yl]-3-(1-{[3,5-dimethyl-7-(2-[3-(methylamino)-3-oxopropyl]amino}ethoxy)tricyclo[3.3.1.13,7]dec-1-yl]methyl)-5-methyl-1H-pyrazol-4-yl)pyridine-2-carboxylic acid (W3.39)

The title compound was prepared as described in Example 1.38, by replacing N,N-dimethylacrylamide with N-methylacrylamide. ¹H NMR (501 MHz, dimethyl sulfoxide-d₆) δ ppm 12.84 (s, 1H), 8.32 (s, 2H), 8.08-7.96 (m, 2H), 7.78 (d, 1H), 7.60 (d, 1H), 7.52-7.40 (m, 3H), 7.39-7.30 (m, 2H), 7.27 (s, 1H), 6.94 (d, 1H), 4.94 (s, 2H), 3.87 (t, 2H), 3.81 (s, 2H), 3.12 (p, 2H), 3.01 (dt, 4H), 2.57 (d, 3H), 2.09 (s, 3H), 1.40 (s, 2H), 1.28 (q, 5H), 1.18-1.07 (m, 4H), 1.02 (q, 2H), 0.85 (s, 7H). MS (ESI) m/e 831.3 (M+H)⁺.

### 1.40 Synthesis of 3-(1-{([3-(2-aminoacetamido)-5,7-dimethyltricyclo[3.3.1.13,7]decan-1-yl]methyl}-5-methyl-1H-pyrazol-4-yl)-6-(8-[(1,3-benzothiazol-2-yl)carbamoyl]-3,4-dihydroisoquinolin-2(1H)-yl)pyridine-2-carboxylic acid (W3.40) 1.40.1 1-((3-bromo-5,7-dimethyladamantan-1-yl)methyl)-5-methyl-1H-pyrazole

To a cooled (−30° C.) solution of Example 1.1.3 (500 mg) in tetrahydrofuran (30 mL) was added n-butyllithium (9.67 mL), and the mixture was stirred at −30° C. for 2 hours. Methyl iodide (1.934 mL) was added dropwise at −30° C. After completion of the addition, the mixture was stirred at −30° C. for additional 2 hours. 1N aqueous HCl in ice water was added slowly, such that the temperature was maintained below 0° C., until the pH reached 6. The mixture was stirred at room temperature for 10 minutes, and was diluted with ice-water (10 mL) and ethyl acetate (20 mL). The layers were separated, and the aqueous was extracted twice with ethyl acetate. The combined organic phases were washed with brine, dried over MgSO₄, filtered and concentrated. The residue was purified by flash silica gel chromatography, eluting with 15/1 to 10/1 petroleum/ethyl acetate, to give the title compound. MS (LC-MS) me 337, 339 (M+H)⁺.

### 1.40.2 1-(3,5-dimethyl-7-((5-methyl-1H-pyrazol-1-yl)methyl)adamantan-1-yl)urea

Example 1.40.1 (2.7 g) and urea (4.81 g) were mixed and stirred at 140° C. for 16 hours. The mixture was cooled to room temperature and suspended in methanol (200 mL×2). The insoluble material was removed by filtration. The filtrate was concentrated to give the title compound. MS (LC-MS) m/e 317.3 (M+H)⁺.

### 1.40.3 3,5-dimethyl-7-((5-methyl-1H-pyrazol-1-yl)methyl)adamantan-1-amine

To a solution of Example 1.40.2 (2.53 g) in 20% ethanol in water (20 mL) was added sodium hydroxide (12.79 g). The mixture was stirred at 120° C. for 16 hours and at 140° C. for another 16 hours. 6N Aqueous HCl was added until the pH reached 6. The mixture was concentrated, and the residue was suspended in methanol (200 mL). The insoluble material was filtered off. The filtrate was concentrated to give the title compound as an HCl salt. MS (LC-MS) m/e 273.9 (M+H)⁺.

### 1.40.4 tert-butyl (2-((3,5-dimethyl-7-((5-methyl-1H-pyrazol-1-yl)methyl)adamantan-1-yl)amino)-2-oxoethyl)carbamate

To a solution of Example 1.40.3 (2.16 g) in N,N-dimethylformamide (100 mL) was added triethylamine (3.30 mL), 2-((tert-butoxycarbonyl)amino)acetic acid (1.799 g) and O-(7-azabenzotriazol-1-yl)-N,N,N′,N′-tetramethyluronium hexafluorophosphate (3.90 g). The mixture was stirred at room temperature for 2 hours. Water (40 mL) was added, and the mixture was extracted with ethyl acetate (70 mL×2). The combined organic phases were washed with brine, dried over sodium sulfate, filtered and concentrated. The residue was purified by silica gel chromatography, eluting with 3/1 to 2′1 petroleum/ethyl acetate, to give the title compound. MS (LC-MS) m/e 430.8 (M+H)⁺.

### 1.40.5 tert-butyl (2-((3-((4-iodo-5-methyl-1H-pyrazol-1-yl)methyl)-5,7-dimethyladamantan-1-yl)amino)-2-oxoethyl)carbamate

To an ambient solution of Example 1.40.4 (1.7 g) in N,N-dimethylformamide (20 mL) was added N-iodosuccinimide (1.066 g) in portions, and the mixture was stirred at room temperature for 16 hours. Ice-water (10 mL) and saturated aqueous Na₂S₂O₃ solution (10 mL) were added. The mixture was extracted with ethyl acetate (30 mL×2). The combined organic phases were washed with brine, dried over sodium sulfate, filtered and concentrated. The residue was purified by silica gel chromatography, eluting with 3/1 to 2/1 petroleum/ethyl acetate, to give the title compound. MS (LC-MS) m/e 556.6 (M+H)⁺.

### 1.40.6 methyl 2-(5-bromo-6-(tert-butoxycarbonyl)pyridin-2-yl)-1,2,3,4-tetrahydroisoquinoline-8-carboxylate

To a solution of methyl 1,2,3,4-tetrahydroisoquinoline-8-carboxylate hydrochloride (12.37 g) and Example 1.4.4 (15 g) in dimethyl sulfoxide (100 mL) was added N,N-diisopropylethylamine (12 mL), and the mixture was stirred at 50° C. for 24 hours. The mixture was then diluted with ethyl acetate (500 mL) and washed with water and brine. The organic layer was dried over sodium sulfate, filtered and concentrated under reduced pressure. The residue was purified by silica gel chromatography, eluting with 20% ethyl acetate in hexane, to give the title compound. MS (ESI) m/e 448.4 (M+H)⁺.

### 1.40.7 methyl 2-(6-(tert-butoxycarbonyl)-5-(4,4,5,5-tetramethyl-1,3,2-dioxaborolan-2-yl)pyridin-2-yl)-1,2,3,4-tetrahydroisoquinoline-8-carboxylate

To a solution of Example 1.40.6 (2.25 g) and [1,1′-bis(diphenylphosphino)ferrocene]dichloropalladium(II) (205 mg) in acetonitrile (30 mL) was added triethylamine (3 mL) and pinacolborane (2 mL), and the mixture was stirred at reflux for 3 hours. The mixture was diluted with ethyl acetate (200 mL) and washed with water and brine. The organic layer was dried over sodium sulfate, filtered and concentrated under reduced pressure. Purification of the residue by flash chromatography, eluting with 20% ethyl acetate in hexane, provided the title compound.

### 1.40.8 methyl 2-(6-(tert-butoxycarbonyl)-5-(1-((3-(2-((tert-butoxycarbonyl)amino)acetamido)-5,7-dimethyladamantan-1-yl)methyl)-5-methyl-1H-pyrazol-4-yl)pyridin-2-yl)-1,2,3,4-tetrahydroisoquinoline-8-carboxylate

The title compound was prepared using the procedure in Example 1.4.7, replacing Example 1.4.6 and Example 1.4.2 with Example 1.40.7 and Example 1.40.5, respectively. MS (ESI) m/e 797.4 (M+H)⁺.

### 1.40.9 2-(6-(tert-butoxycarbonyl)-5-(1-((3-(2-((tert-butoxycarbonyl)amino)acetamido)-5,7-dimethyladamantan-1-yl)methyl)-5-methyl-1H-pyrazol-4-yl)pyridin-2-yl)-1,2,3,4-tetrahydroisoquinoline-8-carboxylic acid

The title compound was prepared using the procedure in Example 1.26.8, replacing Example 1.26.7 with Example 1.40.8. MS (ESI) m/e 783.4 (M+H)⁺.

### 1.40.10 tert-butyl 6-(8-(benzo[d]thiazol-2-ylcarbamoyl)-3,4-dihydroisoquinolin-2(1H)-yl)-3-(1-((3-(2-((tert-butoxycarbonyl)amino)acetamido)-5,7-dimethyladamantan-1-yl)methyl)-5-methyl-1H-pyrazol-4-yl)picolinate

The title compound was prepared using the procedure in Example 1.26.9, replacing Example 1.26.8 with Example 1.40.9. MS (ESI) m/e 915.3 (M+H)⁺.

### 1.40.11 3-(1-{[3-(2-aminoacetamido)-5,7-dimethyltricyclo[3.3.1.13,7]decan-1-yl]methyl}-5-methyl-1H-pyrazol-4-yl)-6-{8-[(1,3-benzothiazol-2-yl)carbamoyl]-3,4-dihydroisoquininolin-2(1H)-yl}pyridine-2-carboxylic acid

The title compound was prepared using the procedure in Example 1.26.10, replacing Example 1.26.9 with Example 1.40.10. ¹H NMR (400 MHz, dimethyl sulfoxide-d₆) δ ppm 12.82 (s, 1H), 8.00 (dd, 1H), 7.90-7.79 (m, 4H), 7.76 (d, 1H), 7.59 (dd, 1H), 7.49-7.38 (m, 3H), 7.37-7.29 (m, 2H), 7.25 (s, 1H), 6.92 (d, 1H), 4.92 (s, 2H), 3.85 (t, 2H), 3.77 (s, 2H), 3.40 (q, 2H), 2.98 (t, 2H), 2.07 (s, 3H), 1.63 (s, 2H), 1.57-1.38 (m, 4H), 1.15-0.93 (m, 6H), 0.80 (s, 6H). MS (ESI) m/c 759.2 (M+H)⁺.

### 1.41 Synthesis of 3-[1-({3-[(2-aminoethyl)sulfanyl]-5,7-dimethyltricyclo[3.3.1.13,7]dec-1-yl}methyl)-5-methyl-1H-pyrazol-4-yl]-6-[8-(1,3-benzothiazol-2-ylcarbamoyl)-3,4-dihydroisoquinolin-2(1H)-yl]pyridine-2-carboxylic acid (W3.41)

### 1.41.1 3-bromo-5,7-dimethyladamantane-1-carboxylic acid

To a solution of bromine (18.75 mL) was added iron (10.19 g) at 0° C., and the mixture was stirred for 30 minutes. 3,5-Dimethyladamantane-1-carboxylic acid (19 g) was added to the above mixture portionwise. The mixture was stirred at room temperature for 36 hours. After adding ice-water (50 mL) and 6N aqueous HCl (100 mL), the mixture was treated with Na₂SO₃ (100 g dissolved in 500 mL water). The aqueous layer was extracted with dichloromethane (300 mL×4). The combined organic layers were washed with 1N aqueous HCl (300 mL) and brine, dried over magnesium sulfate, filtered and concentrated to give the title compound, which was used in the next step without additional purification. ¹H NMR: (400 MHz, CDCl₃) δ ppm 2.23 (s, 2H), 2.01-1.74 (m, 4H), 1.61-1.47 (m, 6H), 0.93 (s, 6H). LC-MS (ESI) m/e 285.0 (M+H)⁺.

### 1.41.2 3-bromo-5,7-dimethyladamantan-1-yl)methanol

To a solution of Example 1.41.1 (10 g) in tetrahydrofuran (20 mL) was added BH3.THF (69.6 mL). The mixture was stirred at room temperature for 16 hours. Upon the completion of the reaction, methanol (20 mL) was added dropwise, and the resulting mixture was stirred for 30 minutes. The mixture was concentrated under reduced pressure. The residue was purified by column chromatography on silica gel, eluting with petroleum ether/ethyl acetate (from 8/1 to 5/1), to give the title compound. ¹H NMR: (400 MHz, CDCl₃) δ ppm 3.28 (s, 2H), 1.98-1.95 (m, 6H), 1.38-1.18 (m, 7H), 0.93 (s, 6H),

### 1.41.3 1-((3-bromo-5,7-dimethyladamantan-1-yl)methyl)-1H-pyrazole

A mixture of 2-(tributylphosphoranylidene)acetonitrile (919 mg), 1H-pyrazole (259 mg) and Example 1.41.2 (800 mg) in toluene (8 mL) was stirred at 90° C. for 16 hours. The mixture was concentrated, and the residue was diluted with ethyl acetate (50 mL). The mixture was washed with brine, dried over magnesium sulfate, filtered and concentrated. The residue was purified by silica gel chromatography, eluting with petroleum ether/ethyl acetate, to give the title compound. LC-MS (ESI) m/e 325.1 (M+H)⁺.

### 1.41.4 3-((1H-pyrazol-1-yl)methyl)-5,7-dimethyladamantane-1-thiol

A mixture of Example 1.41.3 (2.8 g) and thiourea (15.82 g) in 33% (w/w) HBr in acetic acid (50 mL) was stirred at 110° C. for 16 hours and concentrated under reduced pressure to give a residue. The residue was dissolved in 20% ethanol in water (v/v: 200 mL), and sodium hydroxide (19.06 g) was added. The resulting solution was stirred at room temperature for 16 hours and concentrated. The residue was dissolved in water (60 mL), and acidified with 6 N aqueous HCl to pH 5-pH 6. The mixture was extracted with ethyl acetate (200 mL×2). The combined organic layers were washed with brine, dried over MgSO₄, filtered and concentrated to give the title compound. MS (ESI) m/e 319.1 (M+H)⁺.

### 1.41.5 2-((-3-((1H-pyrazol-1-yl)methyl)-5,7-dimethyladamantan-1-yl)thio)ethanol

To a solution of Example 1.41.4 (3.3 g) in ethanol (120 mL) was added sodium ethoxide (2.437 g). The mixture was stirred for 10 minutes, and 2-chloroethanol (1.80 mL) was added dropwise. The mixture was stirred at room temperature for 6 hours and neutralized with 1 N aqueous HCl to pH 7. The mixture was concentrated, and the residue was extracted with ethyl acetate (200 mL×2). The combined organic layers were washed with brine, dried over MgSO₄, filtered and concentrated. The residue was purified by column chromatography on silica gel, eluting with petroleum ether/ethyl acetate from 6/1 to 2/1, to give the title compound. MS (ESI) m/e 321.2 (M+H)⁺.

### 1.41.6 2-((-3,5-dimethyl-7-((5-methyl-1H-pyrazol-1-yl)methyl)adamantan-1-yl)thio)ethanol

To a solution of Example 1.41.5 (2.3 g) in tetrahydrofuran (60 mL) was added n-butyllithium (14.35 mL, 2M in hexane) at −20° C., dropwise under nitrogen. The mixture was stirred for 2 hours. Methyl iodide (4.49 mL) was added to the resulting mixture at −20° C., and the mixture was stirred at −20° C. for 2 hours. The reaction was quenched by the dropwise addition of saturated aqueous NH₄Cl solution at −20° C. The resulting mixture was stirred for 10 minutes and acidified with 1 N aqueous HCl to pH 5. The mixture was extracted with ethyl acetate twice. The combined organic layers were washed with brine, dried over MgSO₄, filtered and concentrated to give the title compound. MS (ESI) m/e 335.3 (M+H)⁺.

### 1.41.7 2-((-3-((4-iodo-5-methyl-1H-pyrazol-1-yl)methyl)-5,7-dimethyladamantan-1-yl)thio)ethanol

To a solution of Example 1.41.6 (3.65 g) in N,N-dimethylformnamide (90 mL) was added N-iodosuccinimide (3.68 g). The mixture was stirred at room temperature for 16 hours. The reaction was quenched by the addition of ice-water (8 mL) and saturated aqueous NaS₂O₃ solution (8 mL). The mixture was stirred for an additional 10 minutes and extracted with ethyl acetate (30 mL×2). The combined organic layers were washed with brine, dried over MgSO₄, filtered and concentrated under reduced pressure. The residue was purified by silica gel chromatography, eluting with petroleum ether/ethyl acetate (6/1 to 3/1), to give the title compound. MS (ESI) m/e 461.2 (M+H)⁺.

### 1.41.8 di-tert-butyl[2-({3-[(4-iodo-5-methyl-1H-pyrazol-1-yl)methyl]-5,7-dimethyltricyclo[3.3.1.13,7]decan-1-yl}sulfanyl)ethyl]-2-imidodicarbonate

To a cold solution (0° C., bath) of Example 1.41.7 (3 g) in dichloromethane (100 mL) was added triethylamine (1.181 mL) and mesyl chloride (0.559 mL). The mixture was stirred at room temperature for 4 hours, and the reaction was quenched by the addition of ice-water (30 mL). The mixture was stirred for an additional 10 minutes and was extracted with dichloromethane (50 mL×2). The combined organic layers were washed with brine, dried over MgSO₄, filtered and concentrated under reduced pressure. The residue was dissolved in acetonitrile (100 mL) and NH(Boc)₂ (1.695 g) and Cs₂CO₃ (4.24 g) were added. The mixture was stirred at 85° C. for 16 hours, and the reaction was quenched by the addition of water (20 mL). The mixture was stirred for 10 minutes and was extracted with ethyl acetate (40 mL×2). The combined organic layers were washed with brine, dried over MgSO₄, filtered and concentrated. The residue was purified by silica gel chromatography, eluting with petroleum ether/ethyl acetate from 10/1 to 6/1, to give the title compound. MS (ESI) m/e 660.1 (M+H)⁺.

### 1.41.9 methyl 2-[5-(1-{[3-({2-[bis(tert-butoxycarbonyl)amino]ethyl}sulfanyl)-5,7-dimethyltricyclo[3.3.1.13,7]decan-1-yl]methyl}-5-methyl-1H-pyrazol-4-yl)-6-(tert-butoxycarbonyl)pyridin-2-yl]-1,2,3,4-tetrahydroisoquinoline-8-carboxylate

The title compound was prepared using the procedure in Example 1.4.7, replacing Example 1.4.6 and Example 1.4.2 with Example 1.40.7 and Example 1.41.8, respectively. LC-MS (ESI) m/e 900.6 (M+H)⁺.

### 1.41.10 2-(6-(tert-butoxycarbonyl)-5-(1-((3-((2-((tert-butoxycarbonyl)amino)ethyl)thio)-5,7-dimethyladamantan-1-yl)methyl)-5-methyl-1H-pyrazol-4-yl)pyridin-2-yl)-1,2,3,4-tetrahydroisoquinoline-8-carboxylic acid

A slurry of lithium hydroxide (553 mg) in water (4.03 mL) and methanol (4 mL) was cooled to 15° C. A solution of Example 1.41.9 (800 mg) in tetrahydrofuran (3.23 mL) and methanol (4 mL) was added slowly, and the reaction was stirred at room temperature. After 18 hours the reaction was cooled in an ice-bath and 1.8 g of phosphoric acid in water (4 mL) was added. The biphasic mixture was transferred to a separatory funnel and extracted with ethyl acetate to give the title compound. LC-MS (ESI) m/e 786.2 (M+H)⁺.

### 1.41.11 tert-butyl 6-(8-(benzo[d]thiazol-2-ylcarbamoyl)-3,4-dihydroisoquinolin-2(1H)-yl)-3-(1-((3-((2-((tert-butoxycarbonyl)amino)ethyl)thio)-5,7-dimethyladamantan-1-yl)methyl)-5-methyl-1H-pyrazol-4-yl)picolinate

A 4 mL amber vial containing Example 1.41.10 (699 mg) was charged with ethyl acetate (5 mL) and 1,1′-carbonyldiimidazole (231 mg) and was stirred for 7 hours at room temperature. A solution of benzo[d]thiazol-2-amine (227 mg) and 1,8-diazabicyclo[5.4.0]undec-7-ene (0.228 mL) in acetonitrile (3 mL) was added, and the reaction was heated to 70° C. After stirring for 18 hours, the reaction was quenched by the addition of 10 mL 1N aqueous HCl and was extracted with ethyl acetate to give the title compound, which was used in the subsequent step without further purification. MS (ESI) m/e 818.2 (M+H)⁺.

### 1.41.12 3-[1-({3-[(2-aminoethyl)sulfanyl]-5,7-dimethyltricyclo[3.3.1.13,7]dec-1-yl}methyl)-5-methyl-1H-pyrazol-4-yl]-6-[8-(1,3-benzothiazol-2-ylcarbamoyl)-3,4-dihydroisoquinolin-2(1H)-yl]pyridine-2-carboxylic acid

To a solution of Example 1.41.11 (510 mg) in dichloromethane (10 mL) was added trifluoroacetic acid (10 mL), and the reaction was stirred at room temperature for 30 minutes. The reaction was quenched with aqueous saturated NaHCO₃ solution and extracted with dichloromethane. The product was purified by reverse-phase HPLC on a Gilson system (C18 column), eluting with 5-80% acetonitrile in water containing 0.1% trifluoroacetic acid, to give the title compound. ¹H NMR (400 MHz, DMSO-d₆) δ ppm 12.86 (bs, 1H), 8.03 (d, 1H), 7.76 (m, 2H), 7.62 (d, 1H), 7.39 (m, 6H), 6.95 (t, 1H), 5.07 (s, 1H), 4.96 (s, 1H), 3.85 (m, 4H), 3.01 (t, 2H), 2.97 (t, 2H), 2.90 (m, 2H), 2.69 (m, 2H), 2.11 (s, 3H), 1.54 (s, 2H), 1.36, (m, 4H), 1.17 (m, 4H), 1.08 (m, 2H), 0.84 (s, 6H). MS (ESI) m/e 762.2 (M+H)⁺.

### 1.42 Synthesis of 3-(1-{[3-(3-aminopropyl)-5,7-dimethyltricyclo[3.3.1.13,7]dec-1-yl]methyl}-5-methyl-1H-pyrazol-4-yl)-6-[8-(1,3-benzothiazol-2-ylcarbamoyl)-3,4-dihydroisoquinolin-2(1H)-yl]pyridine-2-carboxylic acid (W3.42)

### 1.42.1 1-((3-allyl-5,7-dimethyladamantan-1-yl)methyl)-1H-pyrazole

To a solution of Example 1.41.3 (0.825 g) in toluene (5 mL) was added N, N′-azoisobutyronitrile (AIBN, 0.419 g) and allyltributylstannane (2.039 mL). The mixture was purged with N₂ stream for 15 minutes, heated at 80° C. for 8 hours and concentrated. The residue was purified by silica gel chromatography, eluting with 5% ethyl acetate in petroleum ether, to provide the title compound. MS (ESI) m/e 285.2 (M+H)⁺.

### 1.42.2 1-((3-allyl-5,7-dimethyladamantan-1-yl)methyl)-5-methyl-1H-pyrazole

To a solution of Example 1.42.1 (200 mg) in tetrahydrofuran (5 mL) at −78° C. under N₂ was added n-butyllithium (2.81 mL, 2.5 M in hexane). The mixture was stirred for 2 hours while the temperature increased to −20° C. and was stirred at −20° C. for 1 hour. Iodomethane (0.659 mL) was added, and the resulting mixture was stirred for 0.5 hour at −20° C. The reaction was quenched with saturated aqueous NH₄Cl solution and extracted with ethyl acetate twice. The organic layer was washed with brine to give the title compound. MS (ESI) m/e 299.2 (M+H)⁺.

### 1.423 3-(3,5-dimethyl-7-((5-methyl-1H-pyrazol-1-yl)methyl)adamantan-1-yl)propan-1-ol

Under a nitrogen atmosphere, a solution of Example 1.42.2 (2.175 g, 7.29 mmol) in anhydrous tetrahydrofuran (42.5 mL) was cooled to 0° C. BH3-THF (15.30 mL) was added dropwise. The reaction mixture was stirred at room temperature for 2 hours and cooled to 0° C. To the reaction mixture was added 10 N aqueous NaOH (5.03 mL) dropwise, followed by 30 percent H₂O₂(16.52 mL) water solution. The resulting mixture was warmed to room temperature and stirred for 90 minutes. The reaction was quenched with 10 percent aqueous hydrochloric acid (35 mL). The organic layer was separated, and the aqueous layer was extracted with ethyl acetate (2×60 mL). The combined organic layers were washed with brine (3×60 mL) and cooled in an ice bath. A saturated aqueous solution of sodium sulfite (15 mL) was carefully added and the mixture was stirred for a few minutes. The organic layer was dried over sodium sulfate, filtered, and concentrated in vacuo. The residue was purified by silica gel chromatography, eluting with petroleum ether/ethyl acetate (3:1 to 1:1), to provide the title compound. MS (ESI) m/e 317.3 (M+H)⁺.

### 1.42.4 3-(3-((4-iodo-5-methyl-1H-pyrazol-1-yl)methyl)-5,7-dimethyladamantan-1-yl)propan-1-ol

A mixture of Example 1.42.3 (1.19 g) and 1-iodopyrrolidine-2, 5-dione (1.015 g) in N,N-dimethylformamide (7.5 mL) was stirred for 16 hours at room temperature. The reaction was quenched with saturated aqueous Na₂SO₃ solution The mixture was diluted with ethyl acetate and washed with saturated aqueous Na₂SO₃, saturated aqueous Na2CO₃ solution, water and brine. The organic layer was dried over anhydrous Na₂SO₄, filtered, and concentrated. The residue was purified by silica gel chromatography, eluting with petroleum ether/ethyl acetate (3:1 to 1:1), to provide the title compound. MS (ESI) m/e 443.1 (M+H)⁺.

### 1.42.5 3-(3-((4-iodo-5-methyl-1H-pyrazol-1-yl)methyl)-5,7-dimethyladamantan-1-yl)propyl methanesulfonate

To a solution of Example 1.42.4 (1.55 g, 3.50 mmol) in diclioromethane (20 mL) at 0° C. were added triethylamine (0.693 mL) and mesyl chloride (0.374 mL) slowly. The mixture was stirred for 3.5 hours at 20° C., and was diluted with dichloromethane. The organic layer was washed with saturated aqueous NH₄Cl, saturated aqueous NaHCO₃ solution and brine. The organic layer was dried over Na₂SO₄, filtered and concentrated to provide the title compound. MS (ESI) m/e 521.1 (M+H)⁺.

### 1.42.6 di-tert-butyl (3-{3-[(4-iodo-5-methyl-1H-pyrazol-1-yl)methyl]-5,7-dimethyltricyclo[3.3.1.13,7]decan-1-yl}propyl)-2-imidodicarbonate

To a solution of Example 1.42.5 (1.92 g) in acetonitrile (40 mL) at 20° C., were added di-tert-butyl iminodicarbonate (0.962 g) and Cs₂CO₃ (2.404 g). The mixture was stirred for 16 hours at 80° C., and diluted with ethyl acetate, washed with water and brine. The organic layer was dried over Na₂SO₄, filtered and concentrated. The residue was purified by silica gel chromatography, eluting with petroleum ether/ethyl acetate (10:1), to provide the title compound. MS (ESI) m/e 642.3 (M+H)⁺.

### 1.42.7 methyl 2-[5-{1-(3-{3-[bis(tert-butoxycarbonyl)amino]propyl}-5,7-dimethyltricyclo[3.3.1.13,7]decan-1-yl)methyl]-5-methyl-1H-pyrazol-4-yl}-6-(tert-butoxycarbonyl)pyridin-2-yl-1,2,3,4-tetrahydroisoquinoline-8-carboxylate

The title compound was prepared using the procedure in Example 1.4.7, replacing Example 1.4.6 and Example 1.4.2 with Example 1.40.7 and Example 1.42.6, respectively. LC-MS (ESI) m/e 882.6 (M+H)⁺.

### 1.42.8 2-(6-(tert-butoxycarbonyl)-5-(1-((3-(3-((tert-butoxycarbonyl)amino)propyl)-5,7-dimethyladamantan-1-yl)methyl)-5-methyl-1H-pyrazol-4-yl)pyridin-2-yl)-1,2,3,4-tetrahydroisoquinoline-8-carboxylic acid

The title compound was prepared using the procedure in Example 1.41.10 substituting Example 1.42.7 for Example 1.41.9. LC-MS (ESI) m/e 468.5 (M+H)⁺.

### 1.42.9 tert-butyl 6-(8-(benzo[d]thiazol-2-ylcarbamoyl)-3,4-dihydroisoquinolin-2(1H)-yl)-3-(1-((3-(3-((tert-butoxycarbonyl)amino)propyl)-5,7-dimethyladamantan-1-yl)methyl)-5-methyl-1H-pyrazol-4-yl)picolinate

The title compound was prepared using the procedure in Example 1.41.11 substituting Example 1.42.8 for Example 1.41.10.

### 1.42.10 3(1-{[3-(3-aminopropyl)-5,7-dimethyltricyclo[3.3.1.13,7]dec-1-yl]methyl}-5-methyl-1H-pyrazol-4-yl)-6-[8-(1,3-benzothiazol-2-ylcarbamoyl)-3,4-dihydroisoquinolin-2(1H)-yl]pyridine-2-carboxylic acid

The title compound was prepared using the procedure in Example 1.41.12 substituting Example 1.42.9 for Example 1.41.11. ¹H NMR (500 MHz, DMSO-d₆) δ ppm 12.86 (s, 1H), 8.03 (d, 1H), 7.79 (d, 1H), 7.62 (d, 4H), 7.47 (dt, 3H), 7.36 (q, 2H), 7.27 (s, 1H), 6.95 (d, 1H), 4.95 (s, 2H), 3.77 (s, 2H), 3.01 (t, 2H), 2.72 (q, 2H), 2.09 (s, 3H), 1.45 (t, 2H), 1.18-1.05 (m, 9H), 1.00 (d, 6H), 0.80 (s, 6H). MS (ESI) m/e 468.5 (M+H)⁺.

### 1.43 Synthesis of 3-(1-{[3-(2-aminoethoxy)-5,7-dimethyltricyclo[3.3.1.13,7]decan-1-yl]methyl}-5-methyl-1H-pyrazol-4-yl)-6-{5-[(1,3-benzothiazol-2-yl)carbamoyl]quinolin-3-yl}pyridine-2-carboxylic acid (W3.43)

### 1.43.1 methyl 3-bromoquinoline-5-carboxylate

To a solution of 3-bromoquinoline-5-carboxylic acid (2 g) in methanol (30 mL) was added concentrated H₂SO₄ (5 mL). The solution was stirred at reflux overnight. The mixture was concentrated under reduced pressure. The residue was dissolved in ethyl acetate (300 mL) and washed with aqueous Na₂CO₃ solution, water and brine. After drying over anhydrous sodium sulfate, filtration and evaporation of the solvent gave the title product. MS (ESI) m/e 266 (M+H)⁺.

### 1.43.2 methyl 3-(4,4,5,5-tetramethyl-1,3,2-dioxaborolan-2-yl)quinoline-5-carboxylate

To a solution of Example 1.43.1 (356 mg) in N,N-dimethylformamide (5 mL) was added [1,1′-bis(diphenylphosphino)ferrocene]dichloropalladium(II) (55 mg), potassium acetate (197 mg) and bis(pinacolato)diboron (510 mg). The mixture was stirred at 60° C. overnight. The mixture was cooled to room temperature and used in the next reaction without further work up. MS (ESI) m/e 339.2 (M+Na)⁺.

### 1.433 methyl 3-[5-(1-[(3-(2-[bis(tert-butoxycarbonyl)amino]ethoxy)-5,7-dimethyltricyclo[3.3.1.13,7]decan-1-yl)methyl]-5-methyl-1H-pyrazol-4-yl)-6-(tert-butoxycarbonyl)pyridin-2-yl]quinoline-5-carboxylate

To a solution of Example 1.43.2 (626 mg) in 1,4-dioxane (10 mL) and water (5 mL) was added Example 1.23.3 (1.46 g), bis(triphenylphosphine)palladium(II) dichloride (140 mg), and CsF (911 mg). The mixture was stirred at 120° C. for 30 minutes under microwave conditions (Biotage Initiator). The mixture was diluted with ethyl acetate (200 mL), washed with water and brine, dried over anhydrous sodium sulfate, filtered and concentrated. The residue was purified by silica gel chromatography, eluting with 20% ethyl acetate in heptane (1 L) to give the title product. MS (ESI) m/e 880.3 (M+H)⁺.

### 1.43.4 3-(6-(tert-butoxycarbonyl)-5-(1-((3-(2-((tert-butoxycarbonyl)amino)ethoxy)-5,7-dimethyladamantan-1-yl)methyl)-5-methyl-1H-pyrazol-4-yl)pyridin-2-yl)quinoline-5-carboxylic acid

To a solution of Example 1.43.3 (1.34 g) in tetrahydrofuran (10 mL), methanol (5 mL) and water (5 mL) was added LiOH H₂O (120 mg), and the mixture was stirred at room temperature overnight. The mixture was acidified with 2N aqueous HCl, diluted with ethyl acetate (400 mL), washed with water and brine and dried over anhydrous sodium sulfate. Filtration and evaporation of solvent gave the title product. MS (APCI) me 766.3 (M+H)⁺.

### 1.43.5 3-(1-{[3-(2-aminoethoxy)-5,7-dimethyltricyclo[3.3.1.13,7]decan-1-yl]methyl}-5-methyl-1H-pyrazol-4-yl)-6-{5-1[1,3-benzothiazol-2-yl)carbamoyl]quinolin-3-yl}pyridine-2-carboxylic acid

To a solution of Example 1.43.4 (200 mg) in dichloromethane (10 mL) was added benzo[d]thiazol-2-amine (39.2 mg), 1-ethyl-3-[3-(dimethylamino)propyl]-carbodiimide hydrochloride (50 mg) and 4-dimethylaminopyridine (32 mg). The mixture was stirred at room temperature overnight. The reaction mixture was diluted with ethyl acetate (200 mL), washed with water and brine, dried over anhydrous sodium sulfate, filtered and concentrated. The residue was dissolved in dichloromethane and trifluoroacetic acid (10 mL, 1:1), and the reaction was stirred overnight. The mixture was concentrated, and the residue was dissolved in N,N-dimethylformamide (12 mL) and purified by reverse-phase HPLC on a Gilson system (C18 column), eluting with 20-80% acetonitrile in water containing 0.1% trifluoroacetic acid, to give the title product. MS (ESI) m/e 742.1 (M+H)⁺.

### 1.44 Synthesis of 6-[8-(1,3-benzothiazol-2-ylcarbamoyl)-3,4-dihydroisoquinolin-2(1H)-yl]-3-{1-[(3,5-dimethyl-7-{2-[(2-sulfoethyl)amino]ethoxy}tricyclo[3.3.1.13,7]dec-1-yl)methyl]-5-methyl-1H-pyrazol-4-yl}pyridine-2-carboxylic acid (W3.44)

### 1.44.1 tert-butyl 6-[8-(1,3-benzothiazol-2-ylcarbamoyl)-3,4-dihydroisoquinolin-2(1H)-yl]-3-[1-({3,5-dimethyl-7-[(2,2,7,7-tetramethyl-10,10-dioxido-3,3-diphenyl-4,9-dioxa-10□6-thia-13-aza-3-silapentadecan-15-yl)oxy]tricyclo[3.3.1.13,7]dec-1-yl}methyl)-5-methyl-1H-pyrazol-4-yl]pyridine-2-carboxylate

To a solution of Example 1.18.18 (500 mg) in N,N-dimethylformamide (8 mL) was added 4-((tert-butyldiphenylsilyl)oxy)-2,2-dimethylbutyl ethenesulfonate (334 mg). The reaction was stirred at room temperature overnight and methylamine (0.3 mL) was added to quench the reaction. The resulting mixture was stirred for 20 minutes and purified by reverse-phase chromatography using an Analogix system (C18 column), eluting with 50-100% acetonitrile in water containing 0.1% v/v trifluoroacetic acid, to provide the title compound.

### 1.44.2 6-[8-(1,3-benzothiazol-2-ylcarbamoyl)-3,4-dihydroisoquinolin-2(1H)-yl]-3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}pyridine-2-carboxylic acid

Example 1.44.1 (200 mg) in dichloromethane (5 mL) was treated with trifluoroacetic acid (2.5 mL) overnight. The reaction mixture was concentrated and purified by reverse phase chromatography (C18 column), eluting with 20-60% acetonitrile in water containing 0.1% v/v trifluoroacetic acid, to provide the title compound. ¹H NMR (500 MHz, dimethylsulfoxide-d₆) δ ppm 12.86 (s, 1H), 8.32 (s, 2H), 8.02 (d, 1H), 7.78 (d, 1H), 7.60 (d, 1H), 7.51 (d, 1H), 7.40-7.49 (m, 2H), 7.31-7.39 (m, 2H), 7.27 (s, 1H), 6.95 (d, 1H), 4.94 (s, 2H), 3.87 (t, 2H), 3.81 (s, 2H), 3.15-3.25 (m, 2H), 3.03-3.13 (m, 2H), 3.00 (t, 2H), 2.79 (t, 2H), 2.09 (s, 3H), 1.39 (s, 2H), 1.22-1.34 (m, 4H), 0.94-1.18 (m, 6H), 0.85 (s, 6H). MS (ESI) m/e 854.1 (M+H)⁺.

### Example 2. Synthesis of Exemplary Synthons

This example provides synthetic methods for exemplary synthons useful more making ADCs.

### 2.1 Synthesis of N-[6-(2,5-dioxo-2,5-dihydro-1H-pyrrol-1-yl)hexanoyl]-L-valyl-N-{4-[({[2-((3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]phenyl)-N⁵-carbamoyl-L-ornithinamide (Synthon BS)

Example 1.1.14 (72 mg) and 4-((S)-2-((S)-2-(6-(2,5-dioxo-2,5-dihydro-1H-pyrrol-1-yl)hexanamido)-3-methylbutanamido)-5-ureidopentanamido)benzyl (4-nitrophenyl) carbonate (91 mg) in N,N-dimethylformamide (3 mL) was cooled in a water-ice bath and N,N-diisopropylethylamine (0.12 mL) was added. The mixture was stirred at 0° C. for 2 hours and acetic acid (0.057 mL) was added. After concentration of the solvents, the residue was purified via HPLC (20-80% acetonitrile in 0.1% TFA/water) to provide the title compound. ¹H NMR (400 MHz, dimethyl sulfoxide-d₆) δ ppm 9.98 (s, 1H), 8.40 (s, 1H), 8.06 (d, 1H), 8.00 (d, 1H), 7.74-7.89 (m, 4H), 7.59 (d, 2H), 7.46 (s, 2H), 7.37 (t, 1H), 7.18-7.32 (m, 4H), 6.99 (s, 2H), 6.01 (s, 1H), 4.98 (s, 3H), 4.38 (d, 2H), 3.47 (d, 2H), 3.36 (t, 2H), 3.28 (t, 2H), 2.91-3.10 (m, 2H), 2.79-2.91 (m, 4H), 2.19-2.25 (m, 3H), 2.06-2.20 (m, 2H), 1.89-2.02 (m, 3H), 1.53-1.74 (m, 2H), 1.30-1.55 (m, 8H), 1.06-1.29 (m, 10H), 0.91-1.06 (m, 2H), 0.76-0.89 (m, 12H). MS (ESI) m/e 1356.3 (M+H)⁺.

### 2.2 Synthesis of N-[6-(2,5-dioxo-2,5-dihydro-1H-pyrrol-1-yl)hexanoyl]-L-valyl-N-(4-[({[2-((3-[(4-(6-[4-(1,3-benzothiazol-2-ylcarbamoyl)-3,4-dihydro-2H-1,4-benzoxazin-6-yl]-2-carboxypyridin-3-yl}-5-methyl-1H-pyrazol-1-yl)methyl]-5,7-dimethyltricyclo[3.3.1.13,7]dec-1-yl oxy)ethyl](methyl)carbamoyl)oxy)methyl]phenyl)-N5-carbamoyl-L-ornithinamide (Synthon DK)

To a solution of 4-((S)-2-((S)-2-(6-(2,5-dioxo-2,5-dihydro-1H-pyrrol-1-yl)hexanamido)-3-methylbutanamido)-5-ureidopentanamido)benzyl 4-nitrophenyl carbonate (57 mg) and Example 1.2.2 (57 mg) in N,N-dimethylformamide (6 mL) was added N,N-diisopropylethylamine (0.5 mL). The mixture was stirred overnight. The mixture was concentrated under vacuum and the residue was diluted with methanol (3 mL) and acetic acid (0.3 mL), loaded onto a 300 g reverse-phase column, and eluted with 30-70% acetonitrile in 0.1% aqueous TFA solution to provide the title compound. ¹H NMR (400 MHz, dimethyl sulfoxide-d₆) δ ppm 9.97 (s, 1H), 8.73 (d, 1H), 8.07 (d, 1H), 7.90-7.98 (m, 1H), 7.71-7.87 (m, 4H), 7.54-7.63 (m, 2H), 7.45 (d, 1H), 7.32-7.42 (m, 2H), 7.17-7.31 (m, 3H), 6.92-7.03 (m, 3H), 5.88-6.08 (m, 1H), 4.97 (s, 3H), 4.29-4.46 (m, 4H), 4.12-4.26 (m, 4H), 3.86 (s, 3H), 3.21-3.41 (m, 8H), 2.78-3.10 (m, 6H), 2.20 (s, 3H), 1.90-2.18 (m, 3H), 0.92-1.77 (m, 24H), 0.75-0.88 (m, 6H). MS (ESI) m/e 1360.2 (M+H)⁺.

### 2.3 Synthesis of N-[6-(2,5-dioxo-2,5-dihydro-1H-pyrrol-1-yl)hexanoyl]-L-valyl-N-{4-[({[2-{3-[(4-{6-[4-(1,3-benzothiazol-2-ylcarbamoyl)--methyl-1,2,3,4-tetrahydroquinoxalin-6-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)methyl]phenyl}-N5-carbamoyl-L-ornithinamide (Synthon DQ)

The title compound was prepared by substituting Example 1.3.2 for Example 1.2.2 in Example 2.2. ¹H NMR (500 MHz, dimethyl sulfoxide-d₆) δ ppm 9.99 (s, 1H), 8.17-8.35 (m, 1H), 8.07 (d, 1H), 7.89 (d, 1H), 7.71-7.84 (m, 4H), 7.55-7.65 (m, 2H), 7.43 (s, 1H), 7.36 (t, 1H), 7.28 (d, 2H), 7.21 (t, 1H), 6.99 (s, 2H), 6.83 (d, 1H), 5.97 (s, 1H), 5.28-5.51 (m, 2H), 4.98 (s, 2H), 4.32-4.44 (m, 1H), 4.19 (dd, 1H), 3.97-4.13 (m, 2H), 3.85 (s, 2H), 3.29 (d, 3H), 3.00 (s, 3H), 2.80-2.98 (m, 4H), 2.18-2.26 (m, 3H), 1.88-2.17 (m, 3H), 0.91-1.73 (m, 23H), 0.74-0.92 (m, 12H). MS (ESI) m/e 1373.3 (M+H)⁺.

### 2.4 Synthesis of 4-[(1E)-3-({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl beta-D-glucopyranosiduronic acid (Synthon DJ)

### 2.4.1 (E)-tert-butyldimethyl((3-(4,4,5,5-tetramethyl-1,3,2-dioxaborolan-2-yl)allyl)oxy)silane

To a flask charged with tert-butyldimethyl(prop-2-yn-1-yloxy)silane (5 g) and dichloromethane (14.7 mL) under nitrogen atmosphere was added dropwise 4,4,5,5-tetramethyl-1,3,2-dioxaborolane (3.94 g). The mixture was stirred at room temperature for one minute then transferred via cannula to a nitrogen-sparged flask containing Cp₂ZrClH (chloridobis(η5-cyclopentadienyl)hydridozirconium, Schwartz's Reagent) (379 mg). The resulting reaction mixture was stirred at room temperature for 16 hours. The mixture was carefully quenched with water (15 mL), and then extracted with diethyl ether (3×30 mL). The combined organic phases were washed with water (15 mL), dried over MgSO₄, filtered, concentrated and purified by silica gel chromatography, eluting with a gradient from 0-8% ethyl acetate/heptanes to give the title compound. MS (ESI) m/z 316.0 (M+NH₄)⁺.

### 2.4.2 (2S,3R,4S,5S,6S)-2-(4-bromo-2-nitrophenoxy)-6-(methoxycarbonyl)tetrahydro-2H-pyran-3,4,5-triyl triacetate

(2R,3R,4S,5S,6S)-2-Bromo-6-(methoxycarbonyl)tetrahydro-2H-pyran-3,4,5-triyl triacetate (5 g) was dissolved in acetonitrile (100 mL). Ag₂O (2.92 g) was added to the solution and the reaction was stirred for 5 minutes at room temperature. 4-Bromo-2-nitrophenol (2.74 g) was added and the reaction mixture was stirred at room temperature for 4 hours. The silver salt residue was filtered through diatomaceous earth and the filtrate was concentrated under reduced pressure. The residue was purified by silica gel chromatography eluting with a gradient of 10-70% ethyl acetate in heptanes to provide the title compound. MS (ESI+) m/z 550.9 (M+NH₄)⁺.

### 2.4.3 (2S,3R,4S,5S,6S)-2-(4-((E)-3-((tert-butyldimethylsilyl)oxy)prop-1-en-1-yl)-2-nitrophenoxy)-6-(methoxycarbonyl)tetrahydro-2H-pyran-3,4,5-triyl triacetate

Example 2.4.2 (1 g), sodium carbonate (0.595 g), tris(dibenzylideneacetone)dipalladium (Pd₂(dba)₃) (0.086 g), and 1,3,5,7-tetramethyl-6-phenyl-2,4,8-trioxa-6-phosphaadamantane (0.055 g) were combined in a 3-neck 50-mL round bottom flask equipped with a reflux condenser and the system was degassed with nitrogen. Separately, a solution of Example 2.4.1 (0.726 g) in tetrahydrofuran (15 mL) was degassed with nitrogen for 30 minutes. This latter solution was transferred via cannula into the flask containing the solid reagents, followed by addition of degassed water (3 mL) via syringe. The reaction was heated to 60° C. for two hours. The reaction mixture was partitioned between ethyl acetate (3×30 mL) and water (30 mL). The combined organic phases were dried (Na₂SO₄), filtered, and concentrated. The residue was purified by silica gel chromatography, eluting with a gradient from 0-35% ethyl acetate/heptanes to provide the title compound. MS (ESI+) m/z 643.1 (M+NH₄)⁺.

### 2.4.4 (2S,3R,4S,5S,6S)-2-(2-amino-4-((E)-3-hydroxyprop-1-en-1-yl)phenoxy)-6-(methoxycarbonyl)tetrahydro-2H-pyran-3,4,5-triyl triacetate

A 500-mL three-neck, nitrogen-flushed flask equipped with a pressure-equalizing addition funnel was charged with zinc dust (8.77 g). A degassed solution of Example 2.4.3 (8.39 g) in tetrahydrofuran (67 mL) was added via cannula. The resulting suspension was chilled in an ice bath and then 6N aqueous HCl (22.3 mL) was added dropwise via addition funnel at such a rate that the internal temperature of the reaction did not exceed 35° C. After the addition was complete, the reaction mixture was stirred for two hours at room temperature and then filtered through a pad of diatomaceous earth, rinsing with water and ethyl acetate. The filtrate was treated with saturated aqueous NaHCO₃ solution until the water layer was no longer acidic, and the mixture was filtered to remove the resulting solids. The filtrate was transferred to a separatory funnel and the layers were separated. The aqueous layer was extracted with ethyl acetate (3×75 mL) and the combined organic layers were washed with water (100 mL), dried over Na₂SO₄, filtered, and concentrated. The residue was triturated with diethyl ether and the solid was collected by filtration to give the title compound. MS (ESI+) m/z 482.0 (M+H)⁺.

### 2.4.5 (9H-fluoren-9-yl)methyl (3-chloro-3-oxopropyl)carbamate

To a solution of 3-((((9H-fluoren-9-yl)methoxy)carbonyl)amino)propanoic acid (5.0 g) in dichloromethane (53.5 mL) was added sulfurous dichloride (0.703 mL). The mixture was stirred at 60° C. for one hour. The mixture was cooled and concentrated to provide the title compound which was used in the next step without further purification.

### 2.4.6 (2S,3R,4S,5S,6S)-2-(2-(3-((((9H-fluoren-9-yl)methoxy)carbonyl)amino)propanamido)-4-((E)-3-hydroxyprop-1l-en-1-yl)phenoxy)-6-(methoxycarbonyl)tetrahydro-2H-pyran-3,4,5-triyl triacetate

Example 2.4.4 (6.78 g) was dissolved in dichloromethane (50 mL) and the solution was chilled to 0° C., in an ice bath. N,N-Diisopropylethylamine (3.64 g) was added, followed by dropwise addition of a solution of Example 2.4.5 (4.88 g) in dichloromethane (50 mL). The reaction was stirred for 16 hours allowing the ice bath to come to room temperature. Saturated aqueous NaHCO₃ solution (100 mL) was added and the layers were separated. The aqueous layer was further extracted with dichloromethane (2×50 mL). The extracts were dried over Na₂SO₄, filtered, concentrated and then purified by silica gel chromatography, eluting with a gradient of 5-95% ethyl acetate/heptane, to give an inseparable mixture of starting aniline and desired title compound. This mixture was partitioned between 1N aqueous HCl (40 mL) and a 1:1 mixture of diethyl ether and ethyl acetate (40 mL), and then the aqueous phase was further extracted with ethyl acetate (2×25 mL). The organic phases were combined, washed with water (2×25 mL), dried over Na₂SO₄, filtered, and concentrated to give the title compound. MS (ESI+) m/z 774.9 (M+H)⁺.

### 2.4.7 (2S,3R,4S,5S,6S)-2-(2-(3-((((9H-fluoren-9-yl)methoxy)carbonyl)amino)propanamido)-4-((E)-3-(((4-nitrophenoxy)carbonyl)oxy)prop-1-en-1-yl)phenoxy)-6-(methoxycarbonyl)tetrahydro-2H-pyran-3,4,5-triyl triacetate

Example 2.4.6 (3.57 g) was dissolved in dichloromethane (45 mL) and bis(4-nitrophenyl)carbonate (2.80 g) was added, followed by dropwise addition of N,N-diisopropylethylamine (0.896 g). The reaction was stirred at room temperature for two hours. Silica gel (20 g) was then added to the reaction solution and the mixture was concentrated to dryness under reduced pressure, keeping the bath temperature at or below 25° C. The silica residue was loaded atop a column and the crude material was purified by silica gel chromatography, eluting with a gradient from 0-100% ethyl acetate-heptane, providing partially purified title compound which was contaminated with nitrophenol. This material was triturated with methyl tert-butyl ether (250 mL) and the resulting slurry was allowed to sit for 1 hour. The title compound was collected by filtration. Three successive crops were collected in a similar fashion to give the title compound. MS (ESI+) m/z 939.8 (M+H)⁺.

### 2.4.8 3-(1-((3-(2-(((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)(methyl)amino)ethoxy)-5,7-dimethyladamantan-1-yl)methyl)-5-methyl-1H-pyrazol-4-yl)-6-(1-(benzo[d]thiazol-2-ylcarbamoyl)-1,2,3,4-tetrahydroquinolin-7-yl)picolinic acid

Example 1.1.14 (31 mg) and Example 2.4.7 (33.3 mg) in N,N-dimethylformamide (3 mL) at 0° C., was added N,N-diisopropylethylamine (25 μL). The mixture was stirred overnight, diluted with ethyl acetate and washed with water and brine. The organic layer was dried over Na₂SO₄, filtered, and concentrated. The residue was dissolved in methanol (2 mL) and tetrahydrofuran (1 mL), cooled to 0 (C, and 3 M lithium hydroxide aqueous solution (0.35 mL) was added. The mixture was stirred at 0° C. for 4 hours, concentrated and purified by a Gilson HPLC system (C18 column), eluting with 0-60% acetonitrile in 0.1% TFA % water to provide the title compound.

### 2.4.9 4-[(1E)-3-({[12-({3-[(4-6-[1-(1,3-benzothiazol-2-ylcarbamoyl)-1,2,3,4-tetrahydroquinolin-7-yl]-2-carboxypyridin-3-yl)-5-methyl-1H-pyrazol-1-yl)methyl]-5,7-dimethyltricyclo[3.3.1.13,7]dec-1-yl}oxy)ethyl](methyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl beta-D-glucopyranosiduronic acid

To a solution of Example 2.4.8 (19 mg) in N,N-dimethylformamide (2.5 mL) at 0° C., was added 2,5-dioxopyrrolidin-1-yl 6-(2,5-dioxo-2,5-dihydro-1H-pyrrol-1-yl)hexanoate (10 mg) and N,N-diisopropylethylamine (11.08 μL). The mixture was stirred at 0° C. for 15 minutes and a few drops of acetic acid were added. The mixture was purified by a Gilson HPLC system (C18 column), eluting with 20-60% acetonitrile in 0.1% TFA/water to provide the title compound. ¹H NMR (500 MHz, dimethyl sulfoxide-d₆) δ ppm 9.03 (s, 1H), 8.40 (s, 1H), 8.25 (d, 1H), 8.00 (d, 1H), 7.73-7.91 (m, 4H), 7.46 (s, 2H), 7.37 (t, 1H), 7.29 (d, 1H), 7.22 (t, 1H), 7.08-7.13 (m, 1H), 7.04 (d, 1H), 6.98 (s, 2H), 6.56 (d, 1H), 6.10-6.25 (m, 1H), 4.86 (s, 1H), 4.64 (d, 2H), 3.95 (d, 2H), 3.86 (d, 4H), 3.24-3.41 (m, 4H), 2.79-2.96 (m, 6H), 2.54 (t, 2H), 2.21 (s, 3H), 2.03 (t, 2H), 1.90-1.98 (m, 2H), 1.34-1.52 (m, 6H), 1.20-1.30 (m, 5H), 0.89-1.20 (m, 8H), 0.82 (d, 6H). MS (ESI) m/e 1391.2 (M+H)⁺.

### 2.5 Synthesis of 4-[(1E)-3-({[2-((3-[(4-{6-[4-(1,3-benzothiazol-2-ylcarbamoyl)-1-methyl-1,2,3,4-tetrahydroquinoxalin-6-yl]-2-carboxypyridin-3-yl)-5-methyl-1H-pyrazol-1-yl)methyl]-5,7-dimethyltricyclo[3.3.1.13,7]dec-1-yl}oxy)ethyl](methyl)carbamoyl}oxy)prop-1-en-1-yl]-2-(N-[6-(2,5-dioxo-2,5-dihydro-1H-pyrrol-1-yl)hexanoyl]-beta-alanylamino)phenyl beta-D-glucopyranosiduronic acid (Synthon DO)

### 2.5.1 3-(1-((3-(2-((E)-4-(3-(3-((((9H-fluoren-9-yl)methoxy)carbonyl)amino)propanamido)-4-(((2S,3R,4S,5S,6S)-3,4,5-triacetoxy-6-(methoxycarbonyl)tetrahydro-2H-pyran-2-yl)oxy)phenyl)-N-methylbut-3-enamido)ethoxy)-5,7-dimethyladamantan-1-yl)methyl)-5-methyl-1H-pyrazol-4-yl)-6-(4-(benzo[d]thiazol-2-ylcarbamoyl)-1-methyl-1,2,3,4-tetrahydroquinoxalin-6-yl)picolinic acid

To a cold (0° C.) solution of Example 2.4.7 (98 mg) and Example 1.3.2 (91 mg) was added N-ethyl-N-isopropylpropan-2-amine (0.054 mL). The reaction was slowly warmed to room temperature and stirred overnight. The reaction was quenched by the addition of water and ethyl acetate. The layers were separated, and the aqueous was extracted with additional ethyl acetate (2×). The combined organics were dried with anhydrous sodium sulfate, filtered and concentrated under reduced pressure. The residue was used in the subsequent step without further purification. MS (ESI) m/e 1576.8 (M+H)⁺.

### 2.5.2 3-(1-((3-(2-(((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)(methyl)amino)ethoxy)-5,7-dimethyladamantan-1-yl)methyl)-5-methyl-1H-pyrazol-4-yl)-6-(4-(benzo[d]thiazol-2-ylcarbamoyl)-1-methyl-1,2,3,4-tetrahydroquinoxalin-6-yl)picolinic acid

To a solution of Example 2.5.1 (158 mg) in tetrahydrofuran/methanol/water (2:1:1, 4 mL) was added lithium hydroxide monohydrate (20 mg). The reaction mixture was stirred overnight. The mixture was concentrated under vacuum, acidified with TFA, and dissolved in dimethyl sulfoxide/methanol (9 mL) and loaded on an HPLC (Gilson system, eluting with 10-85% acetonitrile in 0.1% TFA in water) for purification to give the pure title compound. MS (ESI) m/e 1228.2 (M+NH)⁺.

### 2.5.3 4-[(1E)-3-({[2-({3-[(4-{6-[4-(1,3-benzothiazol-2-ylcarbamoyl)-1-methyl-1,2,3,4-tetrahydroquinoxalin-6-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl beta-D-glucopyranosiduronic acid

To a solution of Example 2.5.2 (20 mg) and 2,5-dioxopyrrolidin-1-yl 6-(2,5-dioxo-2,5-dihydro-1H-pyrrol-1-yl)hexanoate (6.5 mg) in N,N-dimethylformamide (2 mL) was added N,N-diisopropylethylamine (0.054 mL). The reaction was stirred overnight. The reaction mixture was diluted with methanol (2 mL) and acidified with TFA. The mixture was concentrated and purified on HPLC (Gilson system, eluting with 10-85% acetonitrile in 0.1% TFA in water) to give the pure title compound. ¹H NMR (500 MHz, dimethyl sulfoxide-d₆) δ ppm 9.03 (s, 1H), 8.25 (s, 2H), 7.85-7.95 (m, 2H), 7.72-7.83 (m, 3H), 7.43 (s, 2H), 7.32-7.37 (m, 1H), 7.17-7.25 (m, 1H), 7.08-7.14 (m, 1H), 7.04 (d, 1H), 6.98 (s, 2H), 6.82 (d, 1H), 6.56 (d, 1H), 6.08-6.25 (m, 1H), 4.82-4.92 (m, 1H), 4.64 (d, 3H), 4.00-4.11 (m, 4H), 3.81-3.94 (m, 6H), 3.27-3.50 (m, 17H), 3.00 (s, 3H), 2.83-2.96 (m, 3H), 2.53-2.59 (m, 2H), 2.20 (s, 3H), 2.03 (t, 2H), 1.37-1.55 (m, 4H), 0.90-1.29 (m, 10H), 0.82 (d, 6H). MS (ESI) m/e 1406.2 (M+H)⁺.

### 2.6 Synthesis of 4-[(1E)-3-({[2-({3-[(4-{6-[4-(1,3-benzothiazol-2-ylcarbamoyl)-3,4-dihydro-2H-1,4-benzoxazin-6-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)prop-1-en-1-yl]-2-((N-[6-(2,5-dioxo-2,5-dihydro-1H-pyrrol-1-yl)hexanoyl]-beta-alanylamino)phenyl beta-D-glucopyranosiduronic acid (Synthon DP)

### 2.6.1 3-(1-((3-(2-((E)-4-(3-(3-((((9H-fluoren-9-yl)methoxy)carbonyl)amino)propanamido)-4-(((2S,3R,4S,5S,6S)-3,4,5-triacetoxy-6-(methoxycarbonyl)tetrahydro-2H-pyran-2-yl)oxy)phenyl)-N-methylbut-3-enamido)ethoxy)-5,7-dimethyladamantan-1-yl)methyl)-5-methyl-1H-pyrazol-4-yl)-6-(4-(benzo[d]thiazol-2-ylcarbamoyl)-3,4-dihydro-2H-benzo[b] [1,4]oxazin-6-yl)picolinic acid

To a cold (0° C.) solution of Example 2.4.7 (98 mg) and Example 1.2.2 (91 mg) was added N-ethyl-N-isopropylpropan-2-amine (0.054 mL). The reaction was slowly warmed to room temperature and was stirred overnight. The reaction was quenched by the addition of water and ethyl acetate. The layers were separated, and the aqueous layer was extracted twice with additional ethyl acetate. The combined organics were dried with anhydrous sodium sulfate, filtered and concentrated under reduced pressure. The residue was used in the subsequent step without further purification. MS (ESI) m/e 1547.7 (M+H)⁺.

### 2.6.2 3-(1-((3-(2-(((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)(methyl)amino)ethoxy)-5,7-dimethyladamantan-1-yl)methyl)-5-methyl-1H-pyrazol-4-yl)-6-(4-(benzo[d]thiazol-2-ylcarbamoyl)-3,4-dihydro-2H-benzo[b][1,4]oxazin-6-yl)picolinic acid

The title compound was prepared by substituting Example 2.6.1 for Example 2.5.1 in Example 2.5.2. MS (ESI) m/e 1200.1 (M+NH₄)⁺.

### 2.6.3 4-[(1E)-3-({[2-({3-[(4-{6-[4-(1,3-benzothiazol-2-ylcarbamoyl)-3,4-dihydro-2H-1,4-benzoxazin-6-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl beta-D-glucopyranosiduronic acid

The title compound was prepared by substituting Example 2.6.2 for Example 2.5.2 in Example 2.5.3. ¹H NMR (500 MHz, dimethyl sulfoxide-d₆) δ ppm 9.04 (s, 1H), 8.74 (s, 1H), 8.26 (s, 1H), 7.96 (d, 1H), 7.71-7.92 (m, 4H), 7.35-7.48 (m, 3H), 7.23 (t, 1H), 7.11 (d, 1H), 6.96-7.07 (m, 4H), 6.57 (d, 1H), 6.11-6.24 (m, 1H), 4.81-4.93 (m, 1H), 4.65 (d, 2H), 4.32-4.40 (m, 2H), 4.17 (s, 3H), 3.23-3.51 (m, 14H), 2.83-2.98 (m, 3H), 2.54 (t, 2H), 2.21 (s, 3H), 2.03 (t, 2H), 1.34-1.55 (m, 6H), 0.92-1.31 (m, 13H), 0.82 (d, 6H). MS (ESI) m/e 1415.2 (M+Na)⁺.

### 2.7 Synthesis of 4-[(1E)-3-({[2-((3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl)oxy)prop-1-en-1-yl]-2-({N-[6-(2,5-dioxo-2,5-dihydro-1H-pyrrol-1-yl)hexanoyl]-beta-alanyl}amino)phenyl beta-D-glucopyranosiduronic acid (Synthon HO)

### 2.7.1 3-(1-((3-(2-(((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)(methyl)amino)ethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic acid

To a cold (0° C.) solution of Example 2.4.7 (22 mg) and Example 1.6.3 (20 mg) was added N-ethyl-N-isopropylpropan-2-amine (0.054 mL). The reaction was slowly warmed to room temperature and stirred overnight. The reaction was quenched by the addition of water and ethyl acetate. The layers were separated, and the aqueous layer was extracted twice with additional ethyl acetate. The combined organics were dried with anhydrous sodium sulfate, filtered and concentrated under reduced pressure to give the crude title compound which was dissolved in tetrahydrofuran/methanol/water (2:1:1, 4 mL). Lithium hydroxide monohydrate (40 mg) was added, and the reaction mixture stirred overnight. The mixture was then concentrated under vacuum, acidified with TFA, dissolved in dimethyl sulfoxide/methanol and purified on an HPLC (Gilson system, eluting with 10-85% acetonitrile in 0.1% TFA in water) to give the title compound.

### 2.7.2 4-[(1E)-3-({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl beta-D-glucopyranosiduronic acid

The title compound was prepared by substituting Example 2.7.1 for Example 2.5.2 in Example 2.5.3. ¹H NMR (400 MHz, dimethyl sulfoxide-d₆) δ ppm 13.09 (s, 1H), 9.02 (s, 2H), 8.37 (d, 1H), 8.12-8.29 (m, 4H), 8.06 (s, 1H), 8.02 (d, 1H), 7.93 (d, 1H), 7.76-7.89 (m, 2H), 7.70 (t, 1H), 7.43-7.54 (m, 2H), 7.37 (t, 1H), 7.00-7.13 (m, 2H), 6.98 (s, 2H), 6.56 (d, 1H), 6.08-6.25 (m, 1H), 4.86 (s, 1H), 4.64 (d, 2H), 3.81-3.94 (m, 6H), 3.18-3.51 (m, 12H), 2.78-2.96 (m, 4H), 2.49-2.59 (m, 2H), 2.22 (s, 3H), 2.03 (t, 2H), 1.33-1.54 (m, 6H), 0.93-1.30 (m, 12H), 0.82 (d, 6H). MS (ESI) m/e 1408.3 (M+Na)⁺.

### 2.8 Synthesis of 4-[(1E)-3-({([2-(3-[(4-(6-[8-(1,3-benzothiazol-2-ylcarbamoyl)-3,4-dihydroisoquinolin-2(l H)-yl]-2-carboxypyridin-3-yl)-5-methyl-1H-pyrazol-1-yl)methyl]-5,7-dimethyltricyclo[3.3.1.1˜3,7˜]dec-1-yl}oxy)ethyl](oxetan-3-yl)carbamoyl)oxy)prop-1-en-1-yl]-2-({N-[6-(2,5-dioxo-2,5-dihydro-1H-pyrrol-1-yl)hexanoyl]-beta-alanyl}amino)phenyl beta-D-glucopyranosiduronic acid (Synthon IT)

### 2.8.1 3-(1-(((3-(2-(((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)(oxetan-3-yl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid, Trifluoroacetic Acid

To a solution of Example 1.16.7 (0.039 g) and Example 2.4.7 (0.048 g) in N,N-dimethylformamide (1 mL) was added N,N-diisopropylethylamine (0.037 mL), and the reaction was stirred at room temperature for 2 days. The reaction was concentrated, the residue was re-dissolved in a mixture of methanol (0.5 mL) and tetrahydrofuran (0.5 mL) and treated with lithium hydroxide monohydrate (0.027 g) in water (0.5 mL), and the solution was stirred at room temperature. After stirring for 1 hour, the reaction was quenched with trifluoroacetic acid (0.066 mL), diluted with N,N-dimethylformamide (1 mL), and purified by HPLC using a Gilson system eluting with 10-60% acetonitrile in water containing 0.1% v/v trifluoroacetic acid. The desired fractions were combined and freeze-dried to provide the title compound.

### 2.8.2 4-[(1E)-3-({[2-({3-[(4-(6-[8-(1,3-benzothiazol-2-ylcarbamoyl)-3,4-dihydroisoquinolin-2(1H)-yl]-2-carboxypyridin-3-yl)-5-methyl-1H-pyrazol-1-yl)methyl]-5,7-dimethyltricyclo[3.3.1.13,7]dec-1-yl}oxy)ethyl](oxetan-3-yl)carbamoyl)oxy)prop-1-en-1-yl]-2-((N-[6-(2,5-dioxo-2,5-dihydro-1H-pyrrol-1-yl)hexanoyl]-beta-alanyl}amino)phenyl beta-D-glucopyranosiduronic acid

To a solution of Example 2.8.1 (0.024 g) and 2,5-dioxopyrrolidin-1-yl 6-(2,5-dioxo-2,5-dihydro-1H-pyrrol-1-yl)hexanoate (8.95 mg) in N,N-dimethylformamide (0.5 mL) was added N-ethyl-N-isopropylpropan-2-amine (0.017 mL), and the reaction was stirred at room temperature for 2 hours. The reaction was diluted with N,N-dimethylformamide (1 mL) and water (1 mL) and was purified by HPLC using a Gilson system eluting with 10-60% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.83 (s, 1H), 9.02 (s, 1H), 8.22 (d, 1H), 8.02 (d, 1H), 7.86 (t, 1H), 7.78 (d, 1H), 7.60 (d, 1H), 7.56-7.39 (m, 3H), 7.39-7.30 (m, 2H), 7.27 (s, 1H), 7.14-6.89 (m, 5H), 6.56 (d, 1H), 4.94 (s, 2H), 4.83 (t, 1H), 4.63 (t, 2H), 4.54 (t, 1H), 3.93-3.83 (m, 6H), 3.83-3.75 (m, 4H), 3.33 (dt, 10H), 2.99 (t, 2H), 2.54 (d, 2H), 2.08 (d, 3H), 2.02 (t, 2H), 1.54-0.72 (m, 26H). MS (ESI) m/e 1433.3 (M+H)⁺.

### 2.9 Synthesis of 4-[(1E)-3-({[2-((3-1[(4-6-[8-(1,3-benzothiazol-2-ylcarbamoyl)-5-methoxy-3,4-dihydroisoquinolin-2(1H)-yl]-2-carboxypyridin-3-yl)-5-methyl-1H-pyrazol-1-yl)methyl]-5,7-dimethyltricyclo[3.3.1.13,7]dec-1-yl}oxy)ethyl](2-methoxyethyl)carbamoyl)oxy)prop-1-en-1-yl]-2-({N-[6-(2,5-dioxo-2,5-dihydro-1H-pyrrol-1-yl)hexanoyl]-beta-alanyl}amino)phenyl beta-D-glucopyranosiduronic acid (Synthon KA)

### 2.9.1 3-(1-((3-(2-(((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

Example 1.12.10 (150 mg) was dissolved in N,N-dimethylformamide (0.5 mL), and Example 2.4.7 (190 mg) and N-ethyl-N-isopropylpropan-2-amine (0.30 mL) was added. The reaction was stirred at room temperature overnight. Additional Example 2.4.7 (70 mg) and N,N-diisopropylethylamine (0.10 mL) were added and the reaction was allowed to stir another day. The reaction was then concentrated and the residue was dissolved in tetrahydrofuran (2 mL) and methanol (2 mL), then 1.94N aqueous lithium hydroxide monohydrate (1.0 mL) was added and the mixture was stirred at room temperature for one hour. Purification by reverse phase chromatography (C18 column), eluting with 10-90% acetonitrile in 0.1% TFA/water, provided the title compound as a trifluoroacetic acid salt. MS (ESI) m/e 1270.4 (M−H)⁻.

### 2.9.2 6-(8-(benzo[d]thiazol-2-ylcarbamoyl)-5-methoxy-3,4-dihydroisoquinolin-2(1H)-yl)-3-(1-((3-(2-(((((E)-3-(4-(((2S,3R,4S,5S,6S)-6-carboxy-3,4,5-trihydroxytetrahydro-2H-pyran-2-yl)oxy)-3-(S(6-(2,5-dioxo-2,5-dihydro-1H-pyrrol-1-yl)hexanamido)propanamido)phenyl)allyl)oxy)carbonyl)(2-methoxyethyl)amino)ethoxy)-5,7-dimethyladamantan-1-yl)methyl)-5-methyl-1H-pyrazol-4-yl)picolinic acid

Example 2.9.1 (16 mg) was dissolved in N,N-dimethylformamide (0.3 mL), then 2,5-dioxopyrrolidin-1-yl 6-(2,5-dioxo-2,5-dihydro-1H-pyrrol-1-yl)hexanoate (5 mg) and N-ethyl-N-isopropylpropan-2-amine (11 ILL) were added. The reaction mixture was stirred for three hours at room temperature, and purification by reverse phase chromatography (C18 column), eluting with 10-90% acetonitrile in 0.1% TFA/water, provided the title compound. ¹H NMR (400 MHz, dimethyl sulfoxide-d₆) δ ppm 12.71 (v br s, 1H), 9.03 (s, 1H), 8.25 (s, 1H), 8.01 (d, 1H), 7.87 (br m, 1H), 7.76 (t, 2H), 7.50 (d, 1H), 7.46 (t, 1H), 7.33 (t, 1H), 7.28 (s, 1H), 7.08 (d, 1H), 7.03 (m, 2H), 6.98 (s, 2H), 6.56 (d, 1H), 6.17 (m, 1H), 5.00 (s, 2H), 4.86 (br m, 1H), 4.64 (d, 2H), 3.88 (m, 6H), 3.79 (br m, 2H), 3.43, 3.35 (m, m, total 16H), 3.22 (s, 3H), 2.80 (m, 2H), 2.54 (m, 2H), 2.09 (s, 3H), 2.03 (t, 2H), 1.45 (m, 6H), 1.37 (br m, 2H), 1.28-0.90 (m, 10H), 0.77-0.82 (m, 6H). MS (ESI) m/e 1463.5 (M−H)⁻.

### 2.10 Synthesis of 4-[(1E)-3-({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.13,7]dec-1-yl}oxy)ethyl](2-methoxyethyl)carbamoyl}oxy)prop-1-en-1-yl]-2-({N-[(2,5-dioxo-2,5-dihydro-1H-pyrrol-1-yl)acetyl]-beta-alanyl}amino)phenyl beta-D-glucopyranosiduronic acid (Synthon KB)

Example 2.9.1 (16 mg) was dissolved in N,N-dimethylformamide (0.3 mL), then 2,5-dioxopyrrolidin-1-yl 2-(2,5-dioxo-2,5-dihydro-1H-pyrrol-1-yl)acetate (4 mg) and N-ethyl-N-isopropylpropan-2-amine (11 μL) were added. The reaction mixture was stirred for three hours at room temperature, and purification by reverse phase chromatography (C18 column), eluting with 10-90% acetonitrile in 0.1% TFA/water, provided the title compound. 1H NMR (400 MHz, dimethyl sulfoxide-d6) δ ppm 9.06 (s, 1H), 8.25 (br m, 2H), 8.01 (d, 1H), 7.76 (t, 2H), 7.49 (d, 1H), 7.47 (t, 1H), 7.33 (t, 1H), 7.28 (s, 1H), 7.11 (d, 1H), 7.08 (s, 2H), 7.03 (m, 2H), 6.56 (d, 1H), 6.17 (m, 1H), 5.00 (s, 2H), 4.86 (br m, 1H), 4.64 (d, 2H), 4.02 (s, 2H), 3.88 (m, 6H), 3.79 (br m, 2H), 3.43, 3.35 (m, m, total 14H), 3.22 (s, 3H), 2.80 (m, 2H), 2.57 (m, 2H), 2.09 (s, 3H), 1.37 (br m, 2H), 1.28-0.90 (m, 10H), 0.77-0.82 (m, 6H). MS (ESI) m/e 1407.4 (M−1)−.

### 2.11 Synthesis of 4-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[33, 1.13,7]dec-1-yl}oxy)ethyl](2-methoxyethyl)carbamoyl}oxy)methyl]-3-[2-(2-{[3-(2,5-dioxo-2,5-dihydro-1H-pyrrol-1-yl)propanoyl]amino}ethoxy)ethoxy]phenyl beta-D-glucopyranosiduronic acid (Synthon KT)

### 2.11.1 (2S,3R,4S,5S,6S)-2-(4-formyl-3-hydroxyphenoxy)-6-(methoxycarbonyl)tetrahydro-2H-pyran-3,4,5-triyl triacetate

2,4-Dihydroxybenzaldehyde (15 g) and (2S,3R,4S,5S,6S)-2-bromo-6-(methoxycarbonyl)tetrahydro-2H-pyran-3,4,5-triyl triacetate (10 g) were dissolved in acetonitrile followed by the addition of silver carbonate (10 g) and the reaction was heated to 49° C. After stirring for 4 hours, the reaction was cooled, filtered and concentrated. The crude title compound was suspended in dichloromethane and was filtered through diatomaceous earth and concentrated. The residue was purified by silica gel chromatography, eluting with ethyl acetate/heptane, to provide the title compound.

### 2.11.2 (2S,3R,4S,5S,6S)-2-(3-hydroxy-4-(hydroxymethyl)phenoxy)-6-(methoxycarbonyl)tetrahydro-2H-pyran-3,4,5-triyl triacetate

A solution of Example 2.11.1 (16.12 g) in tetrahydrofuran (200 mL) and methanol (200 mL) was cooled to 0° C., and sodium borohydride (1.476 g) was added portionwise. The reaction was stirred for 20 minutes and quenched with a 1:1 mixture of water:aqueous saturated sodium bicarbonate solution (400 mL). The resulting solids were filtered off and rinsed with ethyl acetate. The phases were separated and the aqueous layer extracted four times with ethyl acetate. The combined organic layers were dried over magnesium sulfate, filtered, and concentrated. The crude title compound was purified via silica gel chromatography eluting with heptane/ethyl acetate to provide the title compound. MS (ESI) m/e 473.9 (M+NH₄)⁺.

### 2.11.3 (2S,3R,4S,5S,6S)-2-(4-(((tert-butyldimethylsilyl)oxy)methyl)-3-hydroxyphenoxy)-6-(methoxycarbonyl)tetrahydro-2H-pyran-3,4,5-triyl triacetate

To Example 2.11.2 (7.66 g) and tert-buty dimethylsilyl chloride (2.78 g) in dichloromethane (168 mL) at −5° C. was added imidazole (2.63 g) and the reaction was stirred overnight allowing the internal temperature of the reaction to warm to 12° C. The reaction mixture was poured into saturated aqueous ammonium chloride and extracted four times with dichloromethane. The combined organics were washed with brine, dried over magnesium sulfate, filtered and concentrated. The crude title compound was purified via silica gel chromatography eluting with heptane/ethyl acetate to provide the title compound. MS (ESI) m/e 593.0 (M+Na)⁺.

### 2.11.4 (2S,3R,4S,5S,6S)-2-(3-(2-(2-((((9H-fluoren-9-yl)methoxy)carbonyl)amino)ethoxy)ethoxy)-4-(((tert-butyldimethylsilyl)oxy)methyl)phenoxy)-6-(methoxycarbonyl)tetrahydro-2H-pyran-3,4,5-triyl triacetate

To Example 2.11.3 (5.03 g) and triphenylphosphine (4.62 g) in toluene (88 mL) was added di-tert-butyl-azodicarboxylate (4.06 g) and the reaction was stirred for 30 minutes. (9H-Fluoren-9-yl)methyl (2-(2-hydroxyethoxy)ethyl)carbamate was added and the reaction was stirred for an additional 1.5 hours. The reaction was loaded directly onto silica gel and was eluted with heptane/ethyl acetate to provide the title compound.

### 2.11.5 (2S,3R,4S,5S,6S)-2-(3-(2-(2-((((9H-fluoren-9-yl)methoxy)carbonyl)amino)ethoxy)ethoxy)-4-(hydroxymethyl)phenoxy)-6-(methoxycarbonyl)tetrahydro-2H-pyran-3,4,5-triyl triacetate

Example 2.11.4 (4.29 g) was stirred in a 3:1:1 solution of acetic acid:water:tetrahydrofuran (100 mL) overnight. The reaction was poured into saturated aqueous sodium bicarbonate and extracted with ethyl acetate. The organic layer was dried over magnesium sulfate, filtered and concentrated. The crude title compound was purified via silica gel chromatography, eluting with heptane/ethyl acetate, to provide the title compound.

### 2.11.6 (2S,3R,4S,5S,6S)-2-(3-(2-(2-((((9H-fluoren-9-yl)methoxy)carbonyl)amino)ethoxy)ethoxy)-4-((((4-nitrophenoxy)carbonyl)oxy)methyl)phenoxy)-6-(methoxycarbonyl)tetrahydro-2H-pyran-3,4,5-triyl triacetate

To a solution of Example 2.11.5 (0.595 g) and bis(4-nitrophenyl) carbonate (0.492 g) in N,N-dimethylformamide (4 mL) was added N-ethyl-N-isopropylpropan-2-amine (0.212 mL). After 1.5 hours, the reaction was concentrated under high vacuum. The reaction was loaded directly onto silica gel and eluted using heptane/ethyl acetate to provide the title compound. MS (ESI) m/e 922.9 (M+Na)⁺.

### 2.11.7 3-(1-((3-(2-((((2-(2-(2-aminoethoxy)ethoxy)-4-(((2S,3R,4S,5S,6S)-6-carboxy-3,4,5-trihydroxytetrahydro-2H-pyran-2-yl)oxy)benzyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

Example 1.12.10 (150 mg) was dissolved in dimethylformamide (0.5 mL). Example 2.11.6 (190 mg) and N,N-diisopropylethylamine (0.30 mL) were added. The reaction was stirred at room temperature overnight. Then more Example 2.11.6 (70 mg) and more N,N-diisopropylethylamine (0.10 mL) were added and the reaction was allowed to stir for another 24 hours. The reaction was then concentrated and the residue was dissolved in tetrahydrofuran (2 mL) and methanol (2 mL), then 1.94N aqueous lithium hydroxide monohydrate (1.0 mL) was added and the mixture stirred at room temperature for one hour. Purification by reverse phase chromatography (C18 column), eluting with 10-90% acetonitrile in 0.1% TFA/water, provided the title compound as a trifluoroacetic acid salt. MS (ESI) m/e 1261.4 (M−H)⁻.

### 2.11.8 6-(8-(benzo[d]thiazol-2-ylcarbamoyl)-5-methoxy-3,4-dihydroisoquinolin-2(l H)-yl)-3-(1-((3-(2-((((4-(((2S,3R,4S,5S,6S)-6-carboxy-3,4,5-trihydroxytetrahydro-2H-pyran-2-yl)oxy)-2-(2-(2-(3-(2,5-dioxo-2,5-dihydro-1H-pyrrol-1-yl)propanamido)ethoxy)ethoxy)benzyl)oxy)carbonyl)(2-methoxyethyl)amino)ethoxy)-5,7-dimethyladamantan-1-yl)methyl)-5-methyl-1H-pyrazol-4-yl)picolinic acid

Example 2.11.7 (19 mg) was dissolved in dimethylformamide (0.3 mL), then 2,5-dioxopyrrolidin-1-yl 3-(2,5-dioxo-2,5-dihydro-1H-pyrrol-1-yl)propanoate (6 mg) and N-ethyl-N-isopropylpropan-2-amine (13 μL) were added. The reaction was stirred for three hours at room temperature, then purification by reverse phase chromatography (C18 column), eluting with 10-90% acetonitrile in 0.1% TFA/water, provided the title compound. ¹H NMR (400 MHz, dimethyl sulfoxide-d₆) δ ppm 12.70 (v br s, 1H), 8.00 (m, 2H), 7.76 (t, 2H), 7.50 (d, 1H), 7.46 (t, 1H), 7.34 (t, 1H), 7.28 (s, 1H), 7.19 (d, 1H), 7.00 (m, 2H), 6.97 (s, 2H), 6.66 (d, 1H), 6.60 (dd, 1H), 5.06 (br m, 1H), 5.00 (s, 2H), 4.96 (s, 2H), 4.09 (m, 2H), 3.88 (m, 6H), 3.80 (br m, 3H), 3.71 (m, 2H), 3.59 (t, 2H), 3.44, 3.38 (both m, total 81f), 3.28 (m, 4H), 3.18 (m, 4H), 2.82 (br m, 2H), 2.33 (t, 2H), 2.09 (s, 3H), 1.33 (br m, 2H), 1.28-0.90 (m, 10H), 0.82 (m, 6H). MS (ESI) m/e 1412.4 (M−H)⁻.

### 2.12 Synthesis of 6-[8-(1,3-benzothiazol-2-ylcarbamoyl)-5-methoxy-3,4-dihydroisoquinolin-2(1H)-yl]-3-(1-{[3-(2-{[({(2E)-3-[4-{[(2S,3R,4S,5S,6S)-6-carboxy-3,4,5-trihydroxytetrahydro-2H-pyran-2-yl]oxy}-3-({3-[({[(2E)-3-(4-{[(2S,3R,4S,5S,6S)-6-carboxy-3,4,5-trihydroxytetrahydro-2H-pyran-2-yl]oxy}-3-[(3-{[3-(2,5-dioxo-2,5-dihydro-1H-pyrrol-1-yl)propanoyl]amino}propanoyl)amino]phenyl)prop-2-en-1-yl]oxy}carbonyl)amino]propanoyl}amino)phenyl]prop-2-en-1-yl}oxy)carbonyl](2-methoxyethyl)amino}ethoxy)-5,7-dimethyltricyclo[3.3.1.13,7]dec-1-yl]methyl}-5-methyl-1H-pyrazol-4-yl)pyridine-2-carboxylic acid (Synthon KU)

### 2.12.1 3-(1-((3-(2-(((((E)-3-(3-(3-(((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)amino)propanamido)-4-(((2S,3R,4S,5S,6S)-6-carboxy-3,4,5-trihydroxytetrahydro-2H-pyran-2-yl)oxy)phenyl)allyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

The title compound was isolated as a by-product during the synthesis of Example 2.9.1. MS (ESI) m/e 1708.5 (M−H)⁻.

### 2.12.2 6-(8-(benzo[d]thiazol-2-ylcarbamoyl)-5-methoxy-3,4-dihydroisoquinolin-2(1H)-yl)-3-(1-((3-(2-(((((E)-3-(4-(((2S,3R,4S,5S,6S)-6-carboxy-3,4,5-trihydroxytetrahydro-2H-pyran-2-yl)oxy)-3-(3-(((((E)-3-(4-(((2S,3R,4S,5S,6S)-6-carboxy-3,4,5-trihydroxytetrahydro-2H-pyran-2-yl)oxy)-3-(3-(3-(2,5-dioxo-2,5-dihydro-1H-pyrrol-1-yl)propanamido)propanamido)phenyl)allyl)oxy)carbonyl)amin o)propanamido)phenyl)allyl)oxy)carbonyl)(2-methoxyethyl)amino)ethoxy)-5,7-dimethyladamantan-1-yl)methyl)-5-methyl-1H-pyrazol-4-yl)picolinic acid

The title compound was prepared by substituting Example 2.12.1 for Example 2.11.7 in Example 2.11.8. ¹H NMR (400 MHz, dimethyl sulfoxide-d₆) δ ppm 8.99 (s, 1H), 8.97 (s, 1H), 8.17 (br s, 2H), 8.00 (br t, 1H), 7.94 (d, 1H), 7.70 (dd, 2H), 7.41 (m, 2H), 7.27 (t, 1H), 7.04 (br d, 2H), 6.97 (d, 2H), 6.93 (m, 2H), 6.89 (s, 2H), 6.52 (d, 1H), 6.49 (d, 1H), 6.11 (m, 2H), 4.93 (s, 2H), 4.80 (in, 2H), 4.56 (m, 4H), 3.83 (m, 7H), 3.72 (br d, 2H), 3.53 (m, 2H), 3.45-3.28 (m, 28H), 3.15 (s, 3H), 2.74 (m, 2H), 2.48 (m, 4H), 2.26 (t, 2H), 2.02 (s, 3H), 1.28 (br d, 2H), 1.17 (m, 4H), 1.02 (m, 4H), 0.89 (in, 2H), 0.2 (m, 6H). MS (ESI−) m/e 1859.5 (M−H)⁻.

### 2.13 Synthesis of 4-[({[2-(2-{2-[({[2-({3-[(4-{6-[8-(1,3-benzothiazol-2-ylcarbainoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.13,7]dec-1-yl}oxy)ethyl](2-methoxyethyl)carbamoyl}oxy)methyl]-5-(beta-D-glucopyranuronosyloxy)phenoxy}ethoxy)ethyl]carbamoyl}oxy)methyl]-3-[2-(2-{[3-(2,5-dioxo-2,5-dihydro-1H-pyrrol-1-yl)propanoyl]amino}ethoxy)ethoxy]phenyl beta-D-glucopyranosiduronic acid (Synthon K(V)

### 2.13.1 3-(1-((3-(2-((((2-(2-(2-((((2-(2-(2-aminoethoxy)ethoxy)-4-(((2S,3R,4S,5S,6S)-6-carboxy-3,4,5-trihydroxytetrahydro-2H-pyran-2-yl)oxy)benzyl)oxy)carbonyl)amino)ethoxy)ethoxy)-4-(((2S,3R,4S,5S,6S)-6-carboxy-3,4,5-trihydroxytetrahydro-2H-pyran-2-yl)oxy)benzyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

The title compound was isolated as a by-product during the synthesis of Example 2.11.7. MS (ESI) m/e 1690.5 (M−H)⁻.

### 2.13.2 6-(8-(benzo[d]thiazol-2-ylcarbamoyl)-5-methoxy-3,4-dihydroisoquinolin-2(1H)-yl)-3-(1-((3-(2-((((4-(((2S,3R,4S,5S,6,S)-6-carboxy-3,4,5-tihydroxytetrahydro-2H-pyran-2-yl)oxy)-2-(2-(2-((((4-(((2S,3R,4S,5S,6S)-6-carboxy-3,4,5-trihydroxytetrahydro-2H-pyran-2-yl)oxy)-2-(2-(2-(3-(2,5-dioxo-2,5-dihydro-1H-pyrrol-1-yl)propanamido)ethoxy)ethoxy)benzyl)oxy)carbonyl)amino)eth oxy)ethoxy)benzyl)oxy)carbonyl)(2-methoxyethyl)amino)ethoxy)-5,7-dimethyladamantan-1-yl)methyl)-:5-methyl-1H-pyrazol-4-yl)picolinic acid

The title compound was prepared by substituting Example 2.13.1 for Example 2.11.7 in Example 2.11.8. ¹H NMR (400 MHz, dimethyl sulfoxide-d₆) δ ppm 8.00 (m, 2H), 7.76 (t, 2H), 7.50 (d, 1H), 7.46 (m, 1H), 7.34 (m, 1H), 7.28 (s, 1H), 7.19 (m, 3H), 6.99 (m, 2H), 6.97 (s, 2H), 6.66 (m, 2H), 6.60 (m, 2H), 5.07 (m, 2H) 5.00 (s, 2H), 4.96 (s, 2H), 4.93 (s, 2H), 4.09 (m, 4H), 3.90 (m, 71f), 3.80 (br d, 4H), 3.71 (m, 4H), 3.59 (t, 2H), 3.48, 3.44, 3.38 (all m, total 14H), 3.28 (m, 7H), 3.16 (m, 7H), 2.81 (br m, 2H), 2.33 (t, 2H), 2.09 (s, 3H), 1.35 (br d, 2H), 1.28-0.90 (m, 10H), 0.82 (m, 6H). MS (ESI) m/c 1842.5 (M−H)⁻.

### 2.14 Synthesis of 4-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.13,7]dec-1-yl}oxy)ethyl](2-methoxyethyl)carbamoyl}oxy)methy]-3-[2-(2-{[(2,5-dioxo-2,5-dihydro-1H-pyrrol-1-yl)acetyl]amino}ethoxy)ethoxy]phenyl beta-D-glucopyranosiduronic acid (Synthon KW)

The title compound was prepared by substituting 2,5-dioxopyrrolidin-1-yl 2-(2,5-dioxo-2,5-dihydro-1H-pyrrol-1-yl)acetate for 2,5-dioxopyrrolidin-1-yl 3-(2,5-dioxo-2,5-dihydro-1H-pyrrol-1-yl)propanoate in Example 2.11.8. ¹H NMR (4(00) MHz, dimethyl sulfoxide-d₆) δ ppm 12.73 (v br s, 1H), 8.21 (br t, 1H), 8.01 (d, 1H), 7.76 (t, 2H), 7.50 (d, 1H), 7.46 (t, 1H), 7.34 (t, 1H), 7.28 (s, 1H), 7.19 (d, 1H), 7.07 (s, 2H), 6.99 (t, 2H), 6.66 (d, 1H), 6.60 (dd, 1H), 5.06 (br m, 1H), 5.00 (s, 2H), 4.96 (s, 2H), 4.09 (m, 2H), 4.02 (s, 2H), 3.88 (m, 6H), 3.80 (br m, 3H), 3.71 (m, 2H), 3.48 (t, 2H), 3.39 (m, 6H), 3.28, 3.21 (both m, 8H), 2.82 (br m, 2H), 2.09 (s, 3H), 1.33 (br m, 2H), 1.28-0.90 (m, 10H), 0.831 (m, 6H). MS (ESI) m/e 1398.4 (M−H)⁻.

### 2.15 Synthesis of 6-[1-(1,3-benzothiazol-2-ylcarbamoyl)-1,2,3,4-tetrahydroquinolin-7-yl]-3-{1-[(3-{[34-(2,5-dioxo-2,5-dihydro-1H-pyrrol-1-yl)-3-methyl-4,32-dioxo-7,10,13,16,19,22,25,28-octaoxa-3,31-diazatetratriacont-1-yl]oxy}-5,7-dimethyltricyclo[3.3.1.13,7]dec-1-yl)methyl]-5-methyl-1H-pyrazol-4-yl}pyridine-2-carboxylic acid (Synthon DC)

To a mixture of Example 1.1.14 (30 mg) and 2,5-dioxopyrrolidin-1-yl 1-(2,5-dioxo-2,5-dihydro-1H-pyrrol-1-yl)-3-oxo-7,10,13,16,19,22,25,28-octaoxa-4-azahentriacontan-3-oate (MAL-dPEG8-NHS-Ester) (40.8 mg) in N,N-dimethylformamide (3 mL) at 0° C., was added N,N-diisopropylethylamine (48 μL). The mixture was stirred at 0° C. for 20 minutes and at room temperature for 10 minutes. Acetic acid (23 μL) was added and the mixture was purified by reverse phase chromatography (C18 column), eluting with 20-60% acetonitrile in 0.1% TFA/water, to provide the title compound. MS (ESI) m/e 1332.5 (M+H)⁺.

### 2.16 Synthesis of 4-1[({[2-({3-[(4-{6-[8-(1,3-benzothiazol-2-ylcarbamoyl)-5-cyano-3,4-dihydroisoquinolin-2(1H)-yl]-2-carboxypyridin-3-yl}-5-methyl-1H-pyrazol-1-yl)methyl]-5,7-dimethyltricyclo[3.3.1.13,7]dec-1-yl}oxy)ethyl]carbamoyl}oxy)methyl]-3-[2-(2-{[3-(2,5-dioxo-2,5-dihydro-1H-pyrrol-1-yl)propanoyl]amino}ethoxy)ethoxy]phenyl beta-D-glucopyranosiduronic acid (Synthon KZ)

### 2.16.1 3-(1-((3-(2-((((2-(2-(2-aminoethoxy)ethoxy)-4-(((2S,3R,4S,5S,6S)-6-carboxy-3,4,5-trihydroxytetrahydro-2H-pyran-2-yl)oxy)benzyl)oxy)carbonyl)amino)ethoxy)-5,7-dimethyladamantan-1-yl)methyl)-5-methyl-1H-pyrazol-4-yl)-6-(8-(benzo[d]thiazol-2-ylcarbamoyl)-5-cyano-3,4-dihydroisoquinolin-2(1H)-yl)picolinic acid

The title compound was prepared by substituting Example 1.13.12 for Example 1.12.10 in Example 2.11.7. MS (ESI) m/e 1200 (M+H)⁺, 1198 (M−H)⁻.

### 2.16.2 4-[({[2-({3-[(4-{6-[8-(1,3-benzothiazol-2-ylcarbamoyl)-5-cyano-3,4-dihydroisoquinolin-2(1H)-yl]-2-carboxypyridin-3-yl}-5-methyl-1H-pyrazol-1-yl)methyl]-5,7-dimethyltricyclo[3.3.1.13,7]dec-1-yl}oxy)ethyl]carbamoyl}oxy)methyl]-3-[2-(2-{[3-(2,5-dioxo-2,5-dihydro-1H-pyrrol-1-yl)propanoyl]amino}ethoxy)ethoxy]phenyl beta-D-glucopyranosiduronic acid

The title compound was prepared by substituting Example 2.16.1 for Example 2.11.7 in Example 2.11.8. ¹H NMR (400 MHz, dimethyl sulfoxide-d₆) δ ppm 13.06 (bs, 2H), 8.04 (d, 1H), 8.01 (t, 1H), 7.92 (d, 1H), 7.78 (dd, 2H), 7.53 (d, 1H), 7.48 (t, 1H), 7.37 (t, 1H), 7.29 (s, 1H), 7.19 (d, 1H), 7.06 (t, 1H), 7.03 (d, 1H), 6.98 (s, 1H), 6.65 (d, 1H), 6.59 (dd, 1H), 5.07 (d, 1H), 4.98 (s, 1H), 4.92 (1H), 4.09 (m, 2H), 3.96 (t, 2H), 3.90 (d, 2H), 3.80 (s, 2H), 3.70 (m, 6H), 3.60 (m, 6H), 3.43 (t, 2H), 3.39 (t, 2H), 3.33 (t, 1H), 3.28 (dd, 1H), 3.16 (m, 4H), 3.03 (q, 2H), 2.33 (t, 2H), 2.09 (s, 3H), 1.37 (s, 2H), 1.25 (q, 4H), 1.11 (q, 4H), 1.00 (dd, 2H), 0.83 (s, 6H). MS (ESI) m/e 1351 (M+H)⁺, 1349 (M−H)⁻.

### 2.17 Synthesis of 4-[(1E)-3-({[2-({3-[(4-(6-[8-(1,3-benzothiazol-2-ylcarbamoyl)-5-methoxy-3,4-dihydroisoquinolin-2(H)-yl]-2-carboxypyridin-3-yl)-5-methyl-1H-pyrazol-1-yl)methyl]-5,7-dimethyltricyclo[3.3.1.13,7]dec-1-yl}oxy)ethyl](2-methoxyethyl)carbamoyl}oxyl)prop-1-en-1-yl]-2-({N-[3-(2,5-dioxo-2,5-dihydro-1H-pyrrol-1-yl)propanoyl]-beta-alanyl}amino)phenyl beta-D-glucopyranosiduronic acid (Synthon LW)

The title compound was prepared by substituting Example 2.9.1 for Example 2.11.7 in Example 2.11.8. ¹H NMR (400 MHz, dimethyl sulfoxide-d₆) δ ppm 9.03 (s, 1H), 8.25 (br m, 1H), 8.05 (br t, 1H), 8.01 (d, 1H), 7.76 (t, 2H), 7.49 (d, 1H), 7.47 (t, 1H), 7.33 (t, 1H), 7.28 (s, 1H), 7.10 (d, 1H), 7.05 (m, 1H), 7.00 (m, 2H), 6.96 (s, 2H), 6.56 (d, 1H), 6.17 (m, 1H), 5.00 (s, 2H), 4.86 (br m, 1H), 4.64 (d, 2H), 3.88 (m, 6H), 3.79 (br m, 2H), 3.60 (t, 2H), 3.43, 3.35 (m, m, total 14H), 3.22 (s, 3H), 2.80 (m, 2H), 2.53 (m, 2H), 2.33 (t, 2H), 2.09 (s, 3H), 1.37 (br m, 2H), 1.28-0.90 (m, 10H), 0.82, 0.77 (both s, total 6H). MS (ESI−) m/e 1421.5 (M−H)⁻.

### 2.18 Synthesis of N-[(2,5-dioxo-2,5-dihydro-1H-pyrrol-1-yl)acetyl]-3-sulfo-L-alanyl-N-{5-[(1E)-3-({[2-(3-[(4-{6-[8-(1,3-benzothiazol-2-ylcarbamoyl)-5-methoxy-3,4-dihydroisoquinolin-2(1H)-yl]-2-carboxypyridin-3-yl-5-methyl-1H-pyrazol-1-yl)methyl]-5,7-dimethyltricyclo[3.3.13,7]dec-1-yl}oxy)ethyl](2-methoxyethyl)carbamoyl}oxy)prop-1-en-1-yl]-2-(beta-D-glucopyranuronosyloxy)phenyl}-beta-alaninamide (Synthon LY)

### 2.18.1 3-(1-((3-(2-(((((E)-3-(3-(3-((R)-2-amino-3-sulfopropanamido)propanamido)-4-(((2S,3R,4S,5S,6S)-6-carboxy-3,4,5-trihydroxytetrahydro-2H-pyran-2-yl)oxy)phenyl)allyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

To a solution of (R)-2-((((9H-fluoren-9-yl)methoxy)carbonyl)amino)-3-sulfopropanoic acid (29 mg) and 2-(3H-[1,2,3]triazolo[4,5-b]pyridin-3-yl)-1.1,3,3-tetramethylisouronium hexafluorophosphate(V) (28 mg) in N,N-dimethylformamide (0.7 mL) was added N,N-diisopropylethylamine (0.013 mL). After stirring for 2 minutes, the reaction was added to a solution of Example 2.9.1 (70 mg) and N-ethyl-N-isopropylpropan-2-amine (0.035 mL) in N,N-dimethylformamide (0.5 mL) at room temperature, and the mixture was stirred for 3 hours. Diethylamine (0.035 mL) was added to the reaction and stirring was continued for an additional 2 hours. The reaction was diluted with water (1 mL), and purified by prep HPLC using a Gilson system eluting with 10-85% acetonitrile in water containing 0.1% v/v trifluoroacetic acid. The desired fractions were combined and freeze-dried to provide the title compound. MS (ESI) m/e 1421.4 (M−H).

### 2.18.2 6-(8-(benzo[d]thiazol-2-ylcarbamoyl)-5-methoxy-3,4-dihydroisoquinolin-2(1H)-yl)-3-(1-((3-(2-(((((E)-3-(4-(((2S,3R,4S,5S,6S)-6-carboxy-3,4,5-trihydroxytetrahydro-2H-pyran-2-yl)oxy)-3-(3-((R)-2-(2-(2,5-dioxo-2,5-dihydro-1H-pyrrol-1-yl)acetamido)-3-sulfopropanamido)propanamido)phenyl)allyl)oxy)carbonyl)(2-methoxyethyl)amino)ethoxy)-5,7-dimethyladamantan-1-yl)methyl)-5-methyl-1H-pyrazol-4-yl)picolinic acid

The title compound was prepared by substituting Example 2.18.1 for Example 2.9.1 in Example 2.10. ¹H NMR (400 MHz, dimethyl sulfoxide-d₆) δ ppm 9.12 (s, 1H), 8.32 (d, 1H), 8.22 (br m, 1H), 8.01 (d, 1H), 7.97 (br t, 1H), 7.76 (t, 2H), 7.49 (d, 1H), 7.47 (t, 1H), 7.33 (t, 1H), 7.28 (s, 1H), 7.10 (d, 1H), 7.07 (s, 2H), 7.05 (m, 1H), 7.00 (m, 2H), 6.56 (d, 1H), 6.17 (m, 1H), 5.00 (s, 2H), 4.86 (br m, 1H), 4.64 (d, 2H), 4.32 (m, 1H), 4.07 (s, 2H), 3.88 (m, 6H), 3.79 (br m, 2H), 3.43, 3.35 (m, m, total 14H), 3.22 (s, 3H), 2.80 (m, 4H), 2.53 (m, 2H), 2.09 (s, 3H), 1.37 (br m, 2H), 1.28-0.90 (m, 10H), 0.82, 0.77 (both s, total 6H). MS (ESI−) m/e 1558.4 (M−H)⁻.

### 2.19 Synthesis of N-[3-(2,5-dioxo-2,5-dihydro-1H-pyrrol-1-yl)propanoyl]-3-sulfo-L-alanyl-N-{5-[(1E)-3-(([2-({3-1(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.13,7 dec-1-yl}oxy)ethyl](2-methoxyethyl)carbamoyl}oxy)prop-1-en-1-yl]-2-(beta-D-glucopyranuronosyloxy)phenyl)-beta-alaninamide (Synthon LZ)

The title compound was prepared by substituting Example 2.18.1 for Example 2.11.7 in Example 2.11.8. ¹H NMR (400 MHz, dimethyl sulfoxide-d₆) δ ppm 9.12 (s, 1H), 8.22 (br m, 1H), 8.07 (br d, 1H), 8.01 (d, 1H), 7.89 (br t, 1H), 7.76 (t, 2H), 7.49 (d, 1H), 7.47 (t, 1H), 7.33 (t, 1H), 7.28 (s, 1H), 7.10 (d, 1H), 7.05 (m, 1H), 7.00 (m, 2H), 6.96 (s, 2H), 6.56 (d, 1H), 6.17 (m, 1H), 5.00 (s, 2H), 4.86 (br m, 1H), 4.64 (d, 2H), 4.32 (m, 1H), 3.88 (m, 6H), 3.79 (br m, 2H), 3.60 (t, 2H), 3.43, 3.35 (m, m, total 14H), 3.22 (s, 3H), 2.80 (m, 4H), 2.53 (m, 2H), 2.37 (m, 2H), 2.09 (s, 3H), 1.37 (br m, 2H), 1.28-0.90 (m, 10H), 0.82, 0.77 (both s, total 6H). MS (ESI−) m/e 1572.5 (M−H)⁻.

### 2.20 Synthesis of N-[(2,5-dioxo-2,5-dihydro-1H-pyrrol-1-yl)acetyl]-beta-alanyl-N-{5-[(1E)-3-({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.13,7]dec-1-yl}oxy)ethyl](2-methoxyethyl)carbamoyl}oxy)prop-1-en-1-yl]-2-(beta-D-glucopyranuronosyloxy)phenyl}-beta-alaninamide (Synthon MB)

### 2.20.1 3-(1-((3-(2-(((((E)-3-(3-(3-(3-aminopropanamido)propanamido)-4-(((2S,3R,4S,5S,6S)-6-carboxy-3,4,5-trihydroxytetrahydro-2H-pyran-2-yl)oxy)phenyl)allyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

The title compound was prepared by substituting 3-((((9H-fluoren-9-yl)methoxy)carbonyl)amino)propanoic acid for (R)-2-((((9H-fluoren-9-yl)methoxy)carbonyl)amino)-3-sulfopropanoic acid in Example 2.18.1. MS (ESI−) m/e 1341.5 (M−H)⁻.

### 2.20.2 6-(8-(benzo[d]thiazol-2-ylcarbamoyl)-5-methoxy-3,4-dihydroisoquinolin-2(1H)-yl)-3-(1-((3-(2-(((((E)-3-(4-(((2S,3R,4S,5S,6S)-6-carboxy-3,4,5-trihydroxytetrahydro-2H-pyran-2-yl)oxy)-3-(3-(3-(2-(2,5-dioxo-2,5-dihydro-1H-pyrrol-1-yl)acetamido)propanamido)propanamido)phenyl)allyl)oxy)car bonyl)(2-methoxyethyl)amino)ethoxy)-5,7-dimethyladamantan-1-yl)methyl)-5-methyl-1H-pyrazol-4-yl)picolinic acid

The title compound was prepared by substituting Example 2.20.1 for Example 2.9.1 in Example 2.10. ¹H NMR (400 MHz, dimethyl sulfoxide-d₆) δ ppm 9.06 (s, 1H), 8.25 (br m, 1H), 8.14 (br t 1H), 8.01 (d, 1H), 7.99 (br m, 1H), 7.76 (t, 2H), 7.49 (d, 1H), 7.47 (t, 1H), 7.33 (t, 1H), 7.28 (s, 1H), 7.10 (d, 1H), 7.07 (s, 2H), 7.05 (m, 1H), 7.00 (m, 2H), 6.56 (d, 1H), 6.17 (m, 1H), 5.00 (s, 2H), 4.86 (br m, 1H), 4.64 (d, 2H), 3.99 (s, 2H), 3.88 (m, 6H), 3.79 (br m, 2H), 3.43, 3.35 (m, m, total 14H), 3.25 (m, 2H), 3.22 (s, 3H), 2.80 (m, 2H), 2.55 (m, 2H), 2.23 (t, 2H), 2.09 (s, 3H), 1.37 (br m, 2H), 1.28-0.90 (m, 10H), 0.82, 0.77 (both s, total 6H). MS (ESI−) m/e 1478.5 (M−H)⁻.

### 2.21 Synthesis of N-[3-(2,5-dioxo-2,5-dihydro-1H-pyrrol-1-yl)propanoyl]-beta-alanyl-N-{5-[(1E)-3-({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.13,7]dec-1-yl}oxy)ethyl](2-methoxyethyl)carbamoyl}oxy)prop-1-en-1-yl]-2-(beta-D-glucopyranuronosyloxy)phenyl}-beta-alaninamide (Synthon MC)

The title compound was prepared by substituting Example 2.20.1 for Example 2.11.7 in Example 2.11.8. ¹H NMR (400 MHz, dimethyl sulfoxide-d₆) δ ppm 9.06 (s, 1H), 8.25 (br m, 1H), 8.01 (d, 1H), 7.94 (br m, 2H), 7.76 (t, 2H), 7.49(d, 1H), 7.47 (t, 1H), 7.33 (t, 1H), 7.28 (s, 1H), 7.10 (d, 1H), 7.05 (m, 1H), 7.00 (m, 2H), 6.97 (s, 2H), 6.56 (d, 1H), 6.17 (m, 1H), 5.00 (s, 2H), 4.86 (br m, 1H), 4.64 (d, 2H), 3.88 (m, 6H), 3.79 (br m, 2H), 3.60 (t, 2H), 3.43, 3.35 (m, m, total 14H), 3.22 (s, 3H), 3.18 (m, 2H), 2.80 (m, 2H), 2.55 (m, 2H), 2.29 (t, 2H), 2.20 (t, 2H), 2.09 (s, 3H), 1.37 (br m, 2H), 1.28-0.90 (m, 10H), 0.82, 0.77 (both s, total 6H). MS (ESI−) m/e 1492.5 (M−H)⁻.

### 2.22 Synthesis of 4-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.13,7]dec-1-yl}oxy)ethyl](2-methoxyethyl)carbamoyl}oxy)methyl]-3-{2-[2-({N-[(2,5-dioxo-2,5-dihydro-1H-pyrrol-1-yl)acetyl]-3-sulfo-L-alanyl}amino)ethoxy]ethoxy}phenyl beta-D-glucopyranosiduronic acid (Synthon ME)

### 2.22.1 3-(1-((3-(2-((((2-(2-(2-((R)-2-amino-3-sulfopropanamido)ethoxy)ethoxy)-4-(((2S,3R,4S,5S,6S)-6-carboxy-3,4,5-trihydroxytetrahydro-2H-pyran-2-yl)oxy)benzyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

The title compound was prepared by substituting Example 2.11.7 for Example 2.9.1 in Example 2.18.1. MS (ESI−) me 1412.4 (M−H)⁻.

### 2.22.2 6-(8-(benzo[d]thiazol-2-ylcarbamoyl)-5-methoxy-3,4-dihydroisoquinolin-2(1H)-yl)-3-(1-((3-(2-((((4-(((2S,3R,4S,5S,6S)-6-carboxy-3,4,5-trihydroxytetrahydro-2H-pyran-2-yl)oxy)-2-(2-(2-((R)-2-(2-(2,5-dioxo-2,5-dihydro-1H-pyrrol-1-yl)acetamido)-3-sulfopropanamido)ethoxy)ethoxy)benzyl)oxy)carbonyl)(2-methoxyethyl)amino)ethoxy)-5,7-dimethyladamantan-1-yl)methyl)-5-methyl-1H-pyrazol-4-yl)picolinic acid

The title compound was prepared by substituting Example 2.22.1 for Example 2.9.1 in Example 2.10. ¹H NMR (400 MHz, dimethyl sulfoxide-d₆) δ ppm 8.32 (d, 1H), 8.02 (d, 1H), 7.76 (m, 3H), 7.52 (d, 1H), 7.46 (t, 1H), 7.34 (t, 1H), 7.30 (s, 1H), 7.19 (d, 1H), 7.06 (s, 2H), 7.00 (m, 2H), 6.66 (d, 1H), 6.58 (dd, 1H), 5.06 (br m, 1H), 5.00 (s, 2H), 4.96 (s, 2H), 4.31 (m, 1H), 4.09 (m, 2H), 4.08 (s, 2H), 3.88 (m, 6H), 3.80 (br m, 4H), 3.71 (m, 2H), 3.44, 3.38 (both m, total 8H), 3.28 (m, 4H), 3.18 (m, 4H), 2.82 (br m, 3H), 2.72 (m, 1H), 2.09 (s, 3H), 1.33 (br m, 2H), 1.28-0.90 (m, 10H), 0.84, 0.81 (both s, total 6H). MS (ESI−) m/e 1549.5 (M−H)⁻.

### 2.23 Synthesis of 4-[({[2-((3-[(4-{6-[8-(1,3-benzothiazol-2-ylcarbamoyl)-5-methoxy-3,4-dihydroisoquinolin-2(1H)-yl]-2-carboxypyridin-3-yl-5-methyl-1H-pyrazol-1-yl)methyl]-5,7-dimethyltricyclo[3.3.1.13,7]dec-1-yl}oxy)ethyl](2-methoxyethyl)carbamoyl}oxy)methyl]-3-{2-[2-({N-[3-(2,5-dioxo-2,5-dihydro-1H-pyrrol-1-yl)propanoyl]-3-sulfo-L-alanyl}amino)ethoxy]ethoxy}phenyl beta-D-glucopyranosiduronic acid (Synthon MF)

The title compound was prepared by substituting Example 2.22.1 for Example 2.11.7 in Example 2.11.8. ¹H NMR (400 MHz, dimethyl sulfoxide-d₆) δ ppm 12.70 (v br s, 1H), 8.06 (d, 1H), 8.02 (d, 1H), 7.76 (m, 3H), 7.52 (d, 1H), 7.46 (t, 1H), 7.34 (t, 1H), 7.30 (s, 1H), 7.19 (d, 1H), 7.00 (m, 2H), 6.95 (s, 2H), 6.66 (d, 1H), 6.58 (dd, 1H), 5.06 (br m, 1H), 5.00 (s, 2H), 4.96 (s, 2H), 4.31 (m, 1H), 4.09 (m, 2H), 3.88 (m, 6H), 3.80 (br m, 4H), 3.71 (m, 2H), 3.59 (t, 2H), 3.44, 3.38 (both m, total 8H), 3.28 (m, 41f), 3.18 (m, 4H), 2.82 (br m, 3H), 2.72 (m, 1H), 2.33 (m, 21f), 2.09 (s, 3H), 1.33 (br m, 2H), 1.28-0.90 (m, 10H), 0.84, 0.81 (both s, total 6H). MS (ESI−) m/e 1563.5 (M−H)⁻,

### 2.24 Synthesis of 4-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.13,7]dec-1-yl}oxy)ethyl](2-methoxyethyl)carbamoyl}oxy)methyl]-3-(2-[2-({N-[(2,5-dioxo-2,5-dihydro-1H-pyrrol-1-yl)acetyl]-beta-alanyl}amino)ethoxy]ethoxy]phenyl beta-D-glucopyranosiduronic acid (Synthon MH)

### 2.24.1 3-(1-((3-(2-((((2-(2-(2-(3-aminopropanamido)ethoxy)ethoxy)-4-(((2S,3R,4S,5S,6S)-6-carboxy-3,4,5-trihydroxytetrahydro-2H-pyran-2-yl)oxy)benzyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

The title compound was prepared by substituting 3-((((9H-fluoren-9-yl)methoxy)carbonyl)amino)propanoic acid for (R)-2-((((9H-fluoren-9-yl)methoxy)carbonyl)amino)-3-sulfopropanoic acid and Example 2.11.7 for Example 2.9.1 in Example 2.18.1. MS (ESI−) m/e 1332.5 (M−H)⁻.

### 2.24.2 6-(8-(benzo[d]thiazol-2-ylcarbamoyl)-5-methoxy-3,4-dihydroisoquinolin-2(1H)-yl)-3-(1-((3-(2-((((4-(((2S,3R,4S,5S,6S)-6-carboxy-3,4,5-trihydroxytetrahydro-2H-pyran-2-yl)oxy)-2-(2-(2-(3-(2-(2,5-dioxo-2,5-dihydro-1H-pyrrol-1-yl)acetamido)propanamido)ethoxy)ethoxy)benzyl)oxy)carbonyl)(2-methoxyethyl)amino)ethoxy)-5,7-dimethyladamantan-1-yl)methyl)-5-methyl-1H-pyrazol-4-yl)picolinic acid

The title compound was prepared by substituting Example 2.24.1 for Example 2.9.1 in Example 2.10. ¹H NMR (400 MHz, dimethyl sulfoxide-d₆) δ ppm 12.70 (v br s, 1H), 8.14 (t, 1H), 8.02 (d, 1H), 7.92 (t, 1H), 7.76 (t, 2H), 7.52 (d, 1H), 7.46 (t, 1H), 7.34 (t, 1H), 7.28 (s, 1H), 7.19 (d, 1H), 7.06 (s, 2H), 7.00 (m, 2H), 6.66 (d, 1H), 6.58 (dd, 1H), 5.06 (br m, 1H), 5.00 (s, 2H), 4.96 (s, 2H), 4.09 (m, 2H), 3.98 (s, 2H), 3.88 (m, 6H), 3.80 (br m, 4H), 3.71 (m, 2H), 3.44, 3.38 (both m, total 81H), 3.28 (m, 4H), 3.18 (m, 6H), 2.82 (br m, 2H), 2.24 (t, 2H), 2.09 (s, 3H), 1.33 (br m, 2H), 1.28-0.90 (m, 10H), 0.84, 0.81 (both s, total 6H). MS (ESI−) m/e 1469.5 (M−H)⁻.

### 2.25 Synthesis of 4-[({[2-({3-[(4-{6-[8-(1,3-benzothiazol-2-ylcarbamoyl)-5-methoxy-3,4-dihydroisoquinolin-2(l H)-yl]-2-carboxypyridin-3-yl}-5-methyl-1H-pyrazol-1-yl)methyl]-5,7-dimethyltricyclo[3.3.1.13,7]dec-1-yl}oxy)ethyl](2-methoxyethyl)carbamoyl}oxy)methyl]-3-{2-[2-((N-[3-(2,5-dioxo-2,5-dihydro-1H-pyrrol-1-yl)propanoyl]-beta-alanyl}amino)ethoxy]ethoxy)phenyl beta-D-glucopyranosiduronic acid (Synthon MI)

The title compound was prepared by substituting Example 2.24.1 for Example 2.11.7 in Example 2.11.8. ¹H NMR (400 MHz, dimethyl sulfoxide-d₆) δ ppm 12.70 (v br s, 1H), 8.02 (d, 1H), 7.94 (t, 1H), 7.88 (t, 1H), 7.76 (t, 2H), 7.52 (d, 1H), 7.46 (t, 1H), 7.34 (t, 1H), 7.28 (s, 1H), 7.19 (d, 1H), 7.00 (m, 2H), 6.95 (s, 2H), 6.66 (d, 1H), 6.58 (dd, 1H), 5.06 (br m, 1H), 5.00 (s, 2H), 4.96 (s, 2H), 4.09 (m, 2H), 3.88 (m, 6H), 3.80 (br m, 4H), 3.71 (m, 2H), 3.59 (t, 2H), 3.44, 3.38 (both m, total 8H), 3.28 (m, 4H), 3.18 (m, 6H), 2.82 (br m, 2H), 2.30 (t, 2H), 2.20 (t, 2H), 2.09 (s, 3H), 1.33 (br m, 2H), 1.28-0.90 (m, 10H), 0.84, 0.81 (both s, total 6H). MS (ESI−) m/e 1483.5 (M−H)⁻.

### 2.26 Synthesis of 2-[({[2-({3-(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.13,7]dec-1-yl}oxy)ethyl](2-methoxyethyl)carbamoyl}oxy)methyl-5-{2-[2-({N-[3-(2,5-dioxo-2,5-dihydro-1H-pyrrol-1-yl)propanoyl]-3-sulfo-L-alanyl}amino)ethoxy]ethoxy}phenyl beta-D-glucopyranosiduronic acid (Synthon NJ)

### 2.26.1 4-(2-(2-bromoethoxy)ethoxy)-2-hydroxybenzaldehyde

A solution of 2,4-dihydroxybenzaldehyde (1.0 g), 1-bromo-2-(2-bromoethoxy)ethane (3.4 g) and potassium carbonate (1.0 g) were stirred together in acetonitrile (30 mL) and heated to 75° C. After stirring for 2 days, the reaction was cooled, diluted with ethyl acetate (100 mL), washed with water (50 mL) and brine (50 mL), dried over magnesium sulfate, filtered and concentrated. Purification via silica gel chromatography, eluting using a gradient of 5-30% ethyl acetate/heptane, provided the title compound. MS (ELSD) m/e 290.4 (M+H)⁺.

### 2.26.2 4-(2-(2-azidoethoxy)ethoxy)-2-hydroxybenzaldehyde

To a solution of Example 2.26.1 (1.26 g) in N,N-dimethylformamide (10 mL) was added sodium azide (0.43 g) and the reaction was stirred at room temperature overnight. The reaction was diluted with diethyl ether (100 mL), washed with water (50 mL) and brine (50 mL), dried over magnesium sulfate, filtered, and concentrated. Purification via silica gel chromatography, eluting with a gradient of 5-30P/o ethyl acetate/heptane, gave the title compound. MS (ELSD) m/c 251.4 (M+H)⁺.

### 2.26.3 (2S,3R,4S,5S,6S)-2-(5-(2-(2-azidoethoxy)ethoxy)-2-formylphenoxy)-6-(methoxycarbonyl)tetrahydro-2H-pyran-3,4,5-triyl triacetate

A solution of Example 2.26.2 (0.84 g), (3R,4S,5S,6S)-2-bromo-6-(methoxycarbonyl)tetrahydro-2H-pyran-3,4,5-triyl triacetate (1.99 g) and silver (I) oxide (1.16 g) were stirred together in acetonitrile (15 mL). After stirring overnight, the reaction was diluted with dichloromethane (20 mL), diatomaceous earth was added and the reaction filtered and concentrated. Purification via silica gel chromatography, eluting with a gradient of 5-75% ethyl acetate/heptane, gave the title compound.

### 2.26.4 (2S,3R,4S,5S,6S)-2-(5-(2-(2-azidoethoxy)ethoxy)-2-(hydroxymethyl)phenoxy)-6-(methoxycarbonyl)tetrahydro-2H-pyran-3,4,5-triyl triacetate

A solution of Example 2.26.3 (0.695 g) in methanol (5 mL) and tetrahydrofuran (2 mL) was cooled to 0° C. Sodium borohydride (0.023 g) was added, and the reaction was warmed to room temperature. After stirring for a total of 1 hour, the reaction was poured into a mixture of ethyl acetate (75 mL) and water (25 mL) and saturated aqueous sodium bicarbonate (10 mL) was added. The organic layer was separated, washed with brine (50 mL), dried over magnesium sulfate, filtered, and concentrated. Purification via silica gel chromatography, eluting with a gradient of 5-85% ethyl acetate/heptane, gave the title compound. MS (ELSD) me 551.8 (M−H₂O)⁻.

### 2.26.5 (2S,3R,4S,5S,6S)-2-(5-(2-(2-aminoethoxy)ethoxy)-2-(hydroxymethyl)phenoxy)-6-(methoxycarbonyl)tetrahydro-2H-pyran-3,4,5-triyl triacetate

To Example 2.26.4 (0.465 g) in tetrahydrofuran (20 mL) was added 5% Pd/C (0.1 g) in a 50 mL pressure bottle and the mixture shaken for 16 hours at 30 psi hydrogen. The reaction was then filtered and concentrated to give the title compound which was used without further purification. MS (ELSD) m/e 544.1 (M+H)⁺.

### 2.26.6 (2S,3R,4S,5S,6S)-2-(5-(2-(2-((((9H-fluoren-9-yl)methoxy)carbonyl)amino)ethoxy)ethoxy)-2-(hydroxymethyl)phenoxy)-6-(methoxycarbonyl)tetrahydro-2H-pyran-3,4,5-triyl triacetate

A solution of Example 2.26.5 (0.443 g) in dichloromethane (8 mL) was cooled to 0° C., then N,N-diisopropylethylamine (0.214 mL) and (9H-fluoren-9-yl)methyl carbonochloridate (0.190 g) were added. After 1 hour, the reaction was concentrated and purified via column chromatography, eluting with 5-95% ethyl acetate:heptane, to give the title compound. MS (ELSD) m/e 748.15 (M-OH)⁻.

### 2.26.7 (2S,3R,4S,5S,6S)-2-(5-(2-(2-((((9H-fluoren-9-yl)methoxy)carbonyl)amino)ethoxy)ethoxy)-2-((((4-nitrophenoxy)carbonyl)oxy)methyl)phenoxy)-6-(methoxycarbonyl)tetrahydro-2H-pyran-3,4,5-triyl triacetate

To a solution of Example 2.26.6 (0.444 g) in N,N-dimethylformamide (5 mL) was added N,N-diisopropylethylamine (0.152 mL) and bis(4-nitrophenyl) carbonate (0.353 g) and the reaction was stirred at room temperature. After 5 hours, the reaction was concentrated and the residue was purified via column chromatography, eluting with 5-90% ethyl acetate/heptane, to give the title compound.

### 2.26.8 3-(1-((3-(2-((((4-(2-(2-aminoethoxy)ethoxy)-2-(((2S,3R,4S,5S,6S)-6-carboxy-3,4,5-trihydroxytetrahydro-2H-pyran-2-yl)oxy)benzyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

Example 1.12.10 (360 mg) was dissolved in dimethylformamide (2.5 mL). Example 2.26.7 (450 mg) and N,N-diisopropylethylamine (0.35 mL) were added. The reaction was stirred at room temperature overnight. The reaction was then concentrated and the residue dissolved in tetrahydrofuran (2.5 mL) and methanol (2.5 mL). Aqueous lithium hydroxide monohydrate (1.94N, 2.2 mL) was added, and the mixture was stirred at room temperature for one hour. Purification by reverse phase chromatography (C18 column), eluting with 10-90% acetonitrile in 0.1% TFA/water, provided the title compound as a trifluoroacetic acid salt. MS (ESI) m/e 1261.4 (M−H)⁻.

### 2.26.9 3-(1-((3-(2-((((4-(2-(2-((R)-2-amino-3-sulfopropanamido)ethoxy)ethoxy)-2-(((2S,3R,4S,5S,6S)-6-carboxy-3,4,5-trihydroxytetrahydro-2H-pyran-2-yl)oxy)benzyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

The title compound was prepared by substituting Example 2.26.8 for Example 2.9.1 in Example 2.18.1. MS (ESI−) m/e 1412.4 (M−H)⁻,

### 2.26.10 6-(8-(benzo[d]thiazol-2-ylcarbamoyl)-5-methoxy-3,4-dihydroisoquinolin-2(1H)-yl)-3-(1-((3-(2-((((2-(((2S,3R,4S,5S,6S)-6-carboxy-3,4,5-trihydroxytetrahydro-2H-pyran-2-yl)oxy)-4-(2-(2-((R)-2-(3-(2,5-dioxo-2,5-dihydro-1H-pyrrol-1-yl)propanamido)-3-sulfopropanamido)ethoxy)ethoxy)benzyl)oxy)carbonyl)(2-methoxyethyl)amino)ethoxy)-5,7-dimethyladamantan-1-yl)methyl)-5-methyl-1H-pyrazol-4-yl)picolinic acid

The title compound was prepared by substituting Example 2.26.9 for Example 2.11.7 in Example 2.11.8. ¹H NMR (400 MHz, dimethyl sulfoxide-d₆) δ ppm 12.70 (v br s, 1H), 8.06 (d, 1H), 8.02 (d, 1H), 7.76 (t, 3H), 7.52 (d, 1H), 7.46 (t, 1H), 7.34 (t, 1H), 7.30 (s, 1H), 7.19 (d, 1H), 7.00 (m, 2H), 6.95 (s, 2H), 6.70 (d, 1H), 6.58 (dd, 1H), 5.06 (br m, 1H), 5.00 (s, 2H), 4.96 (s, 2H), 4.31 (m, 1H), 4.09 (m, 2H), 3.88 (m, 6H), 3.80 (br m, 4H), 3.71 (m, 2H), 3.59 (t, 2H), 3.44, 3.38 (both m, total 8H), 3.28 (m, 4H), 3.18 (m, 4H), 2.82 (br m, 3H), 2.72 (m, 1H), 2.33 (m, 2H), 2.09 (s, 3H), 1.33 (br m, 2H), 1.28-0.90 (m, 10H), 0.84, 0.81 (both s, total 6H). MS (ESI−) m/e 1563.5 (M−H)⁻.

### 2.27 Synthesis of 2-[({[2-({3-[(4-{6-[8-(1,3-benzothiazol-2-ylcarbamoyl)-5-methoxy-3,4-dihydroisoquinolin-2(l H)-yl]-2-carboxypyridin-3-yl}-5-methyl-1H-pyrazol-1-yl)methyl]-5,7-dimethyltricyclo[3.3.1.13,7]dec-1-yl}oxy)ethyl](2-methoxyethyl)carbamoyl}oxy)methyl]-5-{(2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid (Synthon NK)

The title compound was prepared by substituting Example 2.26.9 for Example 2.9.1 in Example 2.9.2. ¹H NMR (400 MHz, dimethyl sulfoxide-d₆) δ ppm 12.70 (v br s, 1H), 8.06 (d, 1H), 8.02 (d, 1H), 7.76 (t, 3H), 7.52 (d, 1H), 7.46 (t, 1H), 7.34 (t, 1H), 7.30 (s, 1H), 7.19 (d, 1H), 7.00 (m, 2H), 6.95 (s, 2H), 6.70 (d, 1H), 6.58 (dd, 1H), 5.06 (br m, 1H), 5.00 (s, 2H), 4.96 (s, 2H), 4.31 (m, 1H), 4.09 (m, 2H), 3.88 (m, 6H), 3.80 (br m, 4H), 3.71 (m, 2H), 3.59 (t, 2H), 3.44, 3.38 (both m, total 8H), 3.28 (m, 4H), 3.18 (m, 4H), 2.82 (br m, 3H), 2.72 (m, 1H), 2.33 (m, 2H), 2.09 (s, 3H), 1.46 (br m, 4H) 1.33 (br m, 2H), 1.28-0.90 (m, 12H), 0.84, 0.81 (both s, total 6H). MS (ESI−) m/e 1605.4 (M−H)⁻.

### 2.28 Synthesis of 4-[({[2-({3-[(4-6-[8-(1,3-benzothiazol-2-ylcarbamoyl)-5-methoxy-3,4-dihydroisoquinolin-2(1H)-yl]-2-carboxypyridin-3-yl)-5-methyl-1H-pyrazol-1-yl)methyl]-5,7-dimethyltricyclo[3.3.13,7]dec-1-yl}oxy)ethyl](2-methoxyethyl)carbamoyl}oxy)methyl]-3-[3-({N-[6-(2,5-dioxo-2,5-dihydro-1H-pyrrol-1-yl)hexanoyl]-3-sulfo-L-alanyl}amino)propoxy]phenyl beta-D-glucopyranosiduronic acid (Synthon NL)

### 2.28.1 (2S,3R,4S,5S,6S)-2-(3-(3-((((9H-fluoren-9-yl)methoxy)carbonyl)amino)propoxy)-4-formylphenoxy)-6-(methoxycarbonyl)tetrahydro-2H-pyran-3,4,5-triyl triacetate

To a solution of (9H-fluoren-9-yl)methyl (3-hydroxypropyl)carbamate (0.245 g) and triphenylphosphine (0.216 g) in tetrahydrofuran (2 mL) at 0° C., was added diisopropyl azodicarboxylate (0.160 mL) dropwise. After stirring for 15 minutes, Example 2.11.1 (0.250 g) was added, the ice bath was removed, and the reaction was allowed to warm to room temperature. After 2 hours, the reaction was concentrated, loaded onto silica gel, and eluted using a gradient of 5-70% ethyl acetate/hexanes to give the title compound. MS (APCI) m/e 512.0 (M-FMOC)⁻.

### 2.28.2 (2S,3R,4S,5S,6S)-2-(3-(3-((((9H-fluoren-9-yl)methoxy)carbonyl)amino)propoxy)-4-(hydroxymethyl)phenoxy)-6-(methoxycarbonyl)tetrahydro-2H-pyran-3,4,5-triyl triacetate

To a suspension of Example 2.28.1 (0.233 g) in methanol (3 mL) and tetrahydrofuran (1 mL) was added sodium borohydride (6 mg). After 30 minutes, the reaction was poured into ethyl acetate (50 mL) and water (25 mL), followed by the addition of sodium bicarbonate (5 mL). The organic layer was separated, washed with brine (25 mL), dried over magnesium sulfate, filtered, and concentrated. Silica gel chromatography, eluting with a gradient of 5-80% ethyl acetate/heptane, gave the title compound. MS (APCI) m/e 718.1 (M−OH)⁻.

### 2.28.3 (2S,3R,4S,5S,6S)-2-(3-(3-((((9H-fluoren-9-yl)methoxy)carbonyl)amino)propoxy)-4-((((4-nitrophenoxy)carbonyl)oxy)methyl)phenoxy)-6-(methoxycarbonyl)tetrahydro-2H-pyran-3,4,5-triyl triacetate

To a solution of Example 2.28.2 (0.140 g) and bis(4-nitrophenyl) carbonate (0.116 g) in N,N-dimethylformamide (1 mL) was added N-ethyl-N-isopropylpropan-2-amine (0.050 mL). After 1.5 hours, the reaction was concentrated under high vacuum, loaded onto silica gel, and eluted using a gradient of 10-70% ethyl acetate/heptane to give the title compound.

### 2.28.4 3-(1-((3-(2-((((2-(3-aminopropoxy)-4-(((2S,3R,4S,5S,6S)-6-carboxy-3,4,5-trihydroxytetrahydro-2H-pyran-2-yl)oxy)benzyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

The title compound was prepared by substituting Example 2.28.3 for Example 2.26.7 in Example 2.26.8. MS (ESI−) m/e 1231.3 (M−H)⁻.

### 2.28.5 3-(1-((3-(2-((((2-(3-((R)-2-amino-3-sulfopropanamido)propoxy)-4-(((2S,3R,4S,5S,6S)-6-carboxy-3,4,5-trihydroxytetrahydro-2H-pyran-2-yl)oxy)benzyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

The title compound was prepared by substituting Example 2.28.4 for Example 2.9.1 in Example 2.18.1. MS (ESI−) m/e 1382.4 (M−H)⁻.

### 2.28.6 6-(8-(benzo[d]thiazol-2-ylcarbamoyl)-5-methoxy-3,4-dihydroisoquinolin-2(1H)-yl)-3-(1-((3-(2-((((4-(((2S,3R,4S,5S,6S)-6-carboxy-3,4,5-trihydroxytetrahydro-2H-pyran-2-yl)oxy)-2-(3-((R)-2-(6-(2,5-dioxo-2,5-dihydro-1H-pyrrol-1-yl)hexanamido)-3-sulfopropanamido)propoxy)benzyl)oxy)carbonyl)(2-methoxyethyl)amino)ethoxy)-5,7-dimethyladamantan-1-yl)methyl)-5-methyl-1H-pyrazol-4-yl)picolinic acid

The title compound was prepared by substituting Example 2.28.5 for Example 2.9.1 in Example 2.9.2. ¹H NMR (400 MHz, dimethyl sulfoxide-d₆) δ ppm 8.01 (d, 1H), 7.85 (m, 2H), 7.76 (m, 2H), 7.52 (d, 1H), 7.46 (t, 1H), 7.34 (m, 1H), 7.30 (s, 1H), 7.16 (d, 1H), 7.00 (m, 3H), 6.97 (s, 2H), 6.64 (d, 1H), 6.56 (dd, 1H), 5.04 (br m, 1H), 5.00 (s, 2H), 4.96 (s, 2H), 4.28 (m, 1H), 3.97 (m, 2H), 3.88 (m, 6H), 3.80 (m, 2H), 3.71 (m, 2H), 3.37 (m, 8H), 3.27 (m, 4H), 3.17 (m, 4H), 2.90-2.65 (m, 4H), 2.09 (s, 3H), 2.05 (t, 2H), 1.81 (m, 2H), 1.46 (br m, 4H), 1.33 (br m, 2H), 1.28-0.90 (m, 12H), 0.84, 0.81 (both s, total 6H). MS (ESI−) me 1575.5 (M−H)⁻.

### 2.29 Synthesis of 4-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)methyl]-3-[3-({N-[6-(2,5-dioxo-2,5-dihydro-1H-pyrrol-1-yl)hexanoyl]-3-sulfo-L-alanyl}amino)propoxy]phenyl beta-D-glucopyranosiduronic acid (Synthon NM)

### 2.29.1 3-(1-((3-(2-((((2-(3-aminopropoxy)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

The title compound was prepared by substituting Example 2.28.3 for Example 2.26.7 and Example 1.9.11 for Example 1.12.10 in Example 2.26.8. MS (ESI−) m/e 1187.4 (M−H)⁻.

### 2.29.2 3-(1-((3-(2-((((2-(3-((R)-2-amino-3-sulfopropanamido)propoxy)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

The title compound was prepared by substituting Example 2.29.1 for Example 2.9.1 in Example 2.18.1. MS (ESI−) m/e 1338.3 (M−H)⁻, 2.293 6-(8-(benzo[d]thiazol-2-ylcarbamol)-5-methoxy-3,4-dihydroisoquinolin-2(1H)-yl)-3-(1-((3-(2-((((4-(((2S,3R,4S,5S,6S)-6-carboxy-3,4,5-trihydroxytetrahydro-2H-pyran-2-yl)oxy)-2-(3-((R)-2-(6-(2,5-dioxo-2,5-dihydro-1H-pyrrol-1-yl)hexanamido)-3-sulfopropanamido)propoxy)benzyl)oxy)carbonyl)(methyl)amin o)ethoxy)-5,7-dimethyladamantantan-1-yl)methyl)-5-methyl-1H-pyrazol-4-yl)picolinic acid

The title compound was prepared by substituting Example 2.29.2 for Example 2.9.1 in Example 2.9.2. ¹H NMR (400 MHz, dimethyl sulfoxide-d₆) δ ppm 8.01 (d, 1H), 7.85 (m, 2H), 7.76 (m, 2H), 7.52 (d, 1H), 7.46 (t, 1H), 7.34 (m, 1H), 7.30 (s, 1H), 7.16 (d, 1H), 7.00 (m, 3H), 6.97 (s, 2H), 6.64 (d, 1H), 6.56 (dd, 1H), 5.04 (br m, 1H), 5.00 (s, 2H), 4.96 (s, 2H), 4.28 (m, 1H), 3.97 (m, 2H), 3.88 (m, 6H), 3.80 (m, 2H), 3.44 (m, 6H), 3.28 (m, 4H), 3.17 (m, 2H), 2.90-2.65 (m, 4H), 2.09 (s, 3H), 2.05 (t, 2H), 1.81 (m, 2H), 1.46 (br m, 4H), 1.33 (br m, 2H), 1.28-0.90 (m, 12H), 0.84, 0.81 (both s, total 6H). MS (ESI−) m/e 1531.5 (M−H)⁻.

### 2.30 Synthesis of N-[6-(2,5-dioxo-2,5-dihydro-1H-pyrrol-1-yl)hexanoyl]-L-valyl-N-{4-[{([(3S)-1-{8-(1,3-benzothiazol-2-ylcarbamoyl)-2-[6-carboxy-5-(1-{[3-(2-methoxyethoxy)-5,7-dimethyltricyclo[3.3.1.13,7]dec-1-yl]methyl}-5-methyl-1H-pyrazol-4-yl)pyridin-2-yl]-1,2,3,4-tetrahydroisoquinolin-6-yl)} pyrrolidin-3-yl]carbamoyl}oxy)methyl]phenyl}-L-alaninamide (Synthon NR)

Example 1.17.10 (40 mg) was dissolved in dimethyl sulfoxide (0.3 mL), and 4-((S)-2-((S)-2-(6-(2,5-dioxo-2,5-dihydro-1H-pyrrol-1-yl)hexanamido)-3-methylbutanamido)propanamido)benzyl (4-nitrophenyl) carbonate (31 mg) and triethylamine (33 μL) were added. The reaction mixture was stirred for 72 hours at room temperature, and purification by reverse phase chromatography (C18 column), eluting with 10-90% acetonitrile in 0.1% TFA water, provided the title compound. MS (ESI) m/e 1357.4 (M+H)⁺, 1355.5 (M−H)⁻.

### 2.31 Synthesis of N-[6-(2,5-dioxo-2,5-dihydro-1H-pyrrol-1-yl)hexanoyl]-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.13,7]dec-1-yl}oxy)ethyl](2-sulfamoylethyl)carbamoyl}oxy)methyl]phenyl}-N5-carbamoyl-L-ornithinamide (Synthon EB)

The title compound was prepared as described in previous examples. ¹H NMR (500 MHz, dimethyl sulfoxide-d₆) δ ppm 12.85 (s, 1H), 9.98 (s, 1H), 8.00-8.09 (m, 2H), 7.78 (t, 2H), 7.61 (t, 3H), 7.40-7.53 (m, 3H), 7.33-7.39 (m, 2H), 7.25-7.30 (m, 3H), 6.86-7.00 (m, 5H), 5.99 (s, 1H), 4.86-5.10 (m, 4H), 4.38 (s, 1H), 4.10-4.26 (m, 1H), 3.88 (t, 2H), 3.80 (d, 2H), 3.33-3.39 (m, 2H), 3.30 (d, 2H), 3.18-3.26 (m, 2H), 2.88-3.06 (m, 5H), 2.04-2.24 (m, 5H), 1.87-2.00 (m, 1H), 1.28-1.74 (m, 10H), 0.89-1.27 (m, 12H), 0.74-0.87 (m, 12H). MS (ESI) m/e 1451.3 (M+H)⁺.

### 2.32 Synthesis of Control Synthon 4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)methyl]-2-({N-[6-(2,5-dioxo-2,5-dihydro-1H-pyrrol-1-yl)hexanoyl]-beta-alanyl}amino)phenyl beta-D-glucopyranosiduronic acid (Synthon H)

### 2.32.1 (2S,3R,4S,5S,6S)-2-(4-formyl-2-nitrophenoxy)-6-(methoxycarbonyl)tetrahydro-2H-pyran-3,4,5-triyl triacetate

To a solution of (2R,3R,4S,5S,6S)-2-bromo-6-(methoxycarbonyl)tetrahydro-2H-pyran-3,4,5-triyl triacetate (4 g) in acetonitrile (100 mL)) was added silver(I) oxide (10.04 g) and 4-hydroxy-3-nitrobenzaldehyde (1.683 g). The reaction mixture was stirred for 4 hours at room temperature and filtered. The filtrate was concentrated, and the residue was purified by silica gel chromatography, eluting with 5-50% ethyl acetate in heptanes, to provide the title compound. MS (ESI) m/e (M+18)⁺.

### 2.32.2 (2S,3R,4S,5S,6S)-2-(4-(hydroxymethyl)-2-nitrophenoxy)-6-(methoxycarbonyl)tetrahydro-2H-pyran-3,4,5-triyl triacetate

To a solution of Example 2.32.1 (6 g) in a mixture of chloroform (75 mL) and isopropanol (18.75 mL) was added 0.87 g of silica gel. The resulting mixture was cooled to 0° C., NaBH₄ (0.470 g) was added, and the resulting suspension was stirred at 0° C. for 45 minutes. The reaction mixture was diluted with dichloromethane (100 mL) and filtered through diatomaceous earth. The filtrate was washed with water and brine and concentrated to give the crude product, which was used without further purification. MS (ESI) m/e (M+NH₄)⁺:

### 2.32.3 (2S,3R,4S,5S,6S)-2-(2-amino-4-(hydroxymethyl)phenoxy)-6-(methoxycarbonyl)tetrahydro-2H-pyran-3,4,5-triyl triacetate

A stirred solution of Example 2.32.2 (7 g) in ethyl acetate (81 mL) was hydrogenated at 20° C. under 1 atmosphere H₂, using 10% Pd/C (1.535 g) as a catalyst for 12 hours. The reaction mixture was filtered through diatomaceous earth, and the solvent was evaporated under reduced pressure. The residue was purified by silica gel chromatography, eluting with 95/5 dichloromethane/methanol, to give the title compound.

### 2.32.4 3-((((9H-fluoren-9-yl)methoxy)carbonyl)amino)propanoic acid

3-Aminopropanoic acid (4.99 g) was dissolved in 10% aqueous Na₂CO₃ solution (120 mL) in a 500 mL flask and cooled with an ice bath. To the resulting solution. (9H-fluoren-9-yl)methyl carbonochloridate (14.5 g) in 1,4-dioxane (100 mL) was gradually added. The reaction mixture was stirred at room temperature for 4 hours, and water (800 mL) was then added. The aqueous phase layer was separated from the reaction mixture and washed with diethyl ether (3×750 mL). The aqueous layer was acidified with 2N HCl aqueous solution to a pH value of 2 and extracted with ethyl acetate (3×750 mL). The organic layers were combined and concentrated to obtain crude product. The crude product was recrystallized in a mixed solvent of ethyl acetate:hexane 1:2 (300 mL) to give the title compound.

### 2.32.5 (9H-fluoren-9-yl)methyl (3-chloro-3-oxopropyl)carbamate

To a solution of Example 2.32.4 in dichloromethane (160 mL) was added sulfurous dichloride (50 mL). The mixture was stirred at 60° C. for 1 hour. The mixture was cooled and concentrated to give the title compound.

### 2.32.6 (2S,3R,4S,5S,6S)-2-(2-(3-((((9H-fluoren-9-yl)methoxy)carbonyl)amino)propanamido)-4-(hydroxymethyl)phenoxy)-6-(methoxycarbonyl)tetrahydro-2H-pyran-3,4,5-triyl triacetate

To a solution of Example 2.32.3 (6 g) in dichloromethane (480 mL) was added N,N-diisopropylethylamine (4.60 mL). Example 2.32.5 (5.34 g) was added, and the mixture was stirred at room temperature for 30 minutes. The mixture was poured into saturated aqueous sodium bicarbonate and was extracted with ethyl acetate. The combined extracts were washed with water and brine and were dried over sodium sulfate. Filtration and concentration gave a residue that was purified via radial chromatography, using 0-100% ethyl acetate in petroleum ether as mobile phase, to give the title compound.

### 2.32.7 (2S,3R,4S,5S,6S)-2-(2-(3-((((9H-fluoren-9-yl)methoxy)carbonyl)amino)propanamido)-4-((((4-nitrophenoxy)carbonyl)oxy)methyl)phenoxy)-6-(methoxycarbonyl)tetrahydro-2H-pyran-3,4,5-triyl triacetate

To a mixture of Example 2.32.6 (5.1 g) in N,N-dimethylformamide (200 mL) was added bis(4-nitrophenyl) carbonate (4.14 g) and N,N-diisopropylethylamine (1.784 mL). The mixture was stirred for 16 hours at room temperature and concentrated under reduced pressure. The crude material was dissolved in dichloromethane and aspirated directly onto a 1 mm radial Chromatotron plate and eluted with 50-1(00)% ethyl acetate in hexanes to give the title compound. MS (ESI) m/e (M+H)⁺.

### 2.32.8 3-bromo-5,7-dimethyladamantanecarboxylic acid

In a 50 mL round-bottomed flask at 0° C., was added bromine (16 mL). Iron powder (7 g) was then added, and the reaction was stirred at 0° C. for 30 minutes. 3,5-Dimethyladamantane-1-carboxylic acid (12 g) was then added. The mixture was warmed up to room temperature and stirred for 3 days. A mixture of ice and concentrated HCl was poured into the reaction mixture. The resulting suspension was treated twice with Na₂SO₃ (50 g in 200 mL water) to destroy bromine and was extracted three times with dichloromethane. The combined organics were washed with 1N aqueous HCl, dried over Na₂SO₄, filtered, and concentrated to give the crude title compound.

### 2.32.9 3-bromo-5,7-dimethyladamantanemethanol

To a solution of Example 2.32.8 (15.4 g) in tetrahydrofuran (200 mL) was added BH₃ (1M in tetrahydrofuran, 150 mL). The mixture was stirred at room temperature overnight. The reaction mixture was then carefully quenched by adding methanol dropwise. The mixture was then concentrated under vacuum, and the residue was balanced between ethyl acetate (500 mL) and 2N aqueous HCl (100 mL). The aqueous layer was further extracted twice with ethyl acetate, and the combined organic extracts were washed with water and brine, dried over Na₂SO₄, and filtered. Evaporation of the solvent gave the title compound.

### 2.32.10 1-((3-bromo-5,7-dimethyltricyclo[3.3.1.13,7]dec-1-yl)methyl)-1H-pyrazole

To a solution of Example 2.32.9 (8.0 g) in toluene (60 mL) was added 1H-pyrazole (1.55 g) and cyanomethylenetributylphosphohorane (2.0 g). The mixture was stirred at 90° C. overnight. The reaction mixture was then concentrated and the residue was purified by silica gel column chromatography (10:1 heptane:ethyl acetate) to give the title compound. MS (ESI) m/e 324.2 (M+H)⁺.

### 2.32.11 2-{[13,5-dimethyl-7-(1H-pyrazol-1-ylmethyl)tricyclo[3.3.1.13,7]dec-1-yl]oxy}ethanol

To a solution of Example 2.32.10 (4.0 g) in ethane-1,2-diol (12 mL) was added triethylamine (3 mL). The mixture was stirred at 150° C. under microwave conditions (Biotage Initiator) for 45 minutes. The mixture was poured into water (100 mL) and extracted three times with ethyl acetate. The combined organic extracts were washed with water and brine, dried over Na₂SO₄, and filtered. Evaporation of the solvent gave the crude product, which was purified by silica gel chromatography, eluting with 20P/o ethyl acetate in heptane, followed by 5% methanol in dichloromethane, to give the title compound. MS (ESI) m/e 305.2 (M+H)⁺.

### 2.32.12 2-({3,5-dimethyl-7-[(5-methyl-1H-pyrazol-1-yl)methyl]tricyclo[3.3.1.13,7]dec-1-yl}oxy)ethanol

To a cooled (−78° C.) solution of Example 2.32.11 (6.05 g) in tetrahydrofuran (100 mL) was added n-BuLi (40 mL, 2.5M in hexane). The mixture was stirred at −78° C. for 1.5 hours. Iodomethane (10 mL) was added through a syringe, and the mixture was stirred at −78° C. for 3 hours. The reaction mixture was then quenched with aqueous NH₄Cl and extracted twice with ethyl acetate, and the combined organic extracts were washed with water and brine. After drying over Na₂SO₄, the solution was filtered and concentrated, and the residue was purified by silica gel column chromatography, eluting with 5% methanol in dichloromethane, to give the title compound. MS (ESI) m/e 319.5 (M+H)⁺.

### 2.32.13 1-({3,5-dimethyl-7-[2-(hydroxy)ethoxy]tricyclo[3.3.1.13,7]dec-1-yl}methyl)-4-iodo-5-methyl-1H-pyrazole

To a solution of Example 2.32.12 (3.5 g) in N,N-dimethylformamide (30 mL) was added N-iodosuccinimide (3.2 g). The mixture was stirred at room temperature for 1.5 hours. The reaction mixture was then diluted with ethyl acetate (600 mL) and washed with aqueous NaHSO₃, water, and brine. After drying over Na₂SO₄, the solution was filtered and concentrated and the residue was purified by silica gel chromatography (20% ethyl acetate in dichloromethane) to give the title compound. MS (ESI) m/e 445.3 (M+H)⁺.

### 2.32.14 2-({3-[(4-iodo-5-methyl-1H-pyrazol-1-yl)methyl]-5,7-dimethyltricyclo[3.3.1.13,7]dec-1-yl}oxy)ethyl methanesulfonate

To a cooled solution of Example 2.32.13 (6.16 g) in dichloromethane (100 mL) was added triethylamine (4.21 g) followed by methanesulfonyl chloride (1.6 g). The mixture was stirred at room temperature for 1.5 hours. The reaction mixture was then diluted with ethyl acetate (600 mL) and washed with water and brine. After drying over Na₂SO₄, the solution was filtered and concentrated, and the residue was used in the next reaction without further purification. MS (ESI) m/e 523.4 (M+H)⁺.

### 2.32.15 1-{3,5-dimethyl-7-[2-(methylamino)ethoxy]tricyclo[3.3.1.13,7]dec-1-yl}methyl)-4-iodo-5-methyl-1H-pyrazole

A solution of Example 2.32.14 (2.5 g) in 2M methylamine in methanol (15 mL) was stirred at 100° C. for 20 minutes under microwave conditions (Biotage Initiator). The reaction mixture was concentrated under vacuum. The residue was then diluted with ethyl acetate (400 mL) and washed with aqueous NaHCO₃, water and brine. After drying over Na₂SO₄, the solution was filtered and concentrated, and the residue was used in the next reaction without further purification. MS (ESI) m/e 458.4 (M+H)⁺.

### 2.32.16 tert-butyl [2-({3-[(4-iodo-5-methyl-1H-pyrazol-1-yl)methyl]-5,7-dimethyltricyclo[3.3.1.13,7]dec-1-yl}oxy)ethyl]methylcarbamate

To a solution of Example 2.32.15 (2.2 g) in tetrahydrofuran (30 mL) was added di-tert-butyl dicarbonate (1.26 g) and a catalytic amount of 4-dimethylaminopyridine. The mixture was stirred at room temperature for 1.5 hours and diluted with ethyl acetate (300 mL). The solution was washed with saturated aqueous NaHCO₃, water (60 mL), and brine (60 mL). The organic layer was dried with Na₂SO₄, filtered, and concentrated. The residue was purified by silica gel chromatography, eluting with 20% ethyl acetate in dichloromethane, to give the title compound. MS (ESI) m/e 558.5 (M+H)⁺.

### 2.32.17 6-fluoro-3-bromopicolinic acid

A slurry of 6-amino-3-bromopicolinic acid (25 g) in 400 mL 1:1 dichloromethane/chloroform was added to nitrosonium tetrafluoroborate (18.2 g) in dichloromethane (100 mL) at 5° C. over 1 hour, and the resulting mixture was stirred for another 30 minutes, then warmed to 35° C., and stirred overnight. The reaction was cooled to room temperature, and then adjusted to pH 4 with aqueous NaH₂PO₄ solution. The resulting solution was extracted three times with dichloromethane, and the combined extracts were washed with brine, dried over sodium sulfate, filtered and concentrated to provide the title compound.

### 2.32.18 Tert-butyl 3-bromo-6-fluoropicolinate

Para-toluenesulfonyl chloride (27.6 g) was added to a solution of Example 2.32.17 (14.5 g) and pyridine (26.7 mL) in dichloromethane (100 mL) and tert-butanol (80 mL) at 0° C. The reaction was stirred for 15 minutes, warmed to room temperature, and stirred overnight. The solution was concentrated and partitioned between ethyl acetate and aqueous Na₂CO₃ solution. The layers were separated, and the aqueous layer extracted with ethyl acetate. The organic layers were combined, rinsed with aqueous Na₂CO₃ solution and brine, dried over sodium sulfate, filtered, and concentrated to provide the title compound.

### 2.32.19 methyl 2-(5-bromo-6-(tert-butoxycarbonyl)pyridin-2-yl)-1,2,3,4-tetrahydroisoquinoline-8-carboxylate

To a solution of methyl 1,2,3,4-tetrahydroisoquinoline-8-carboxylate hydrochloride (12.37 g) and Example 2.32.18 (15 g) in dimethyl sulfoxide (100 mL) was added N,N-diisopropylethylamine (12 mL). The mixture was stirred at 50° C. for 24 hours. The mixture was then diluted with ethyl acetate (500 mL), washed with water and brine, and dried over Na₂SO₄. Filtration and evaporation of the solvent gave a residue that was purified by silica gel chromatography, eluting with 20% ethyl acetate in heptane, to give the title compound. MS (ESI) m/e 448.4 (M+H)⁺.

### 232.20 methyl 2-(6-(tert-butoxycarbonyl)-5-(4,4,5,5-tetramethyl-1,3,2-dioxaborolan-2-yl)pyridin-2-yl)-1,2,3,4-tetrahydroisoquinoline-8-carboxylate

To a solution of Example 2.32.19 (2.25 g) and [1,1′-bis(diphenylphosphino)ferrocene]dichloropalladium(II) (205 mg) in acetonitrile (30 mL) was added triethylamine (3 mL) and pinacolborane (2 mL). The mixture was stirred at reflux for 3 hours. The mixture was diluted with ethyl acetate (200 mL) and washed with water and brine, and dried over Na₂SO₄. Filtration, evaporation of the solvent, and silica gel chromatography (eluted with 20% ethyl acetate in heptane) gave the title compound. MS (ESI) m/e 495.4 (M+H)⁺.

### 232.21 methyl 2-(6-(tert-butoxycarbonyl)-5-(1-((3-(2-((tert-butoxycarbonyl)(methyl)amino)ethoxy)-5,7-dimethyltricyclo[3.3.1.13,7]dec-1-yl)methyl)-5-methyl-1H-pyrazol-4-yl)pyridin-2-yl)-1,2,3,4-tetrahydroisoquinoline-8-carboxylate

To a solution of Example 2.32.20 (4.94 g) in tetrahydrofuran (60 mL) and water (20 mL) was added Example 2.32.16 (5.57 g), 1,3,5,7-tetramethyl-8-tetradecyl-2,4,6-trioxa-8-phosphaadamantane (412 mg), tris(dibenzylideneacetone)dipalladium(0) (457 mg), and K₃PO₄ (11 g). The mixture was stirred at reflux for 24 hours. The reaction mixture was cooled, diluted with ethyl acetate (5(00) mL), washed with water and brine, and dried over Na₂SO₄. Filtration and evaporation of the solvent gave a residue that was purified by silica gel chromatography, eluting with 2(P % ethyl acetate in heptane, to give the title compound. MS (ESI) m e 799.1 (M+H)⁺.

### 2.32.22 2-(6-(tert-butoxycarbonyl)-5-(1-((3-(2-((tert-butoxycarbonyl)(methyl)amino)ethoxy)-5,7-dimethyltricyclo[3.3.1.13,7]dec-1-yl)methyl)-5-methyl-1H-pyrazol-4-yl)pyridin-2-yl)-1,2,3,4-tetrahydroisoquinoline-8-carboxylic acid

To a solution of Example 2.32.21 (10 g) in tetrahydrofuran (60 mL), methanol (30 mL) and water (30 mL) was added lithium hydroxide monohydrate (1.2 g). The mixture was stirred at room temperature for 24 hours. The reaction mixture was neutralized with 2% aqueous HCl and concentrated under vacuum. The residue was diluted with ethyl acetate (800 mL) and washed with water and brine, and dried over Na₂SO₄. Filtration and evaporation of the solvent gave the title compound. MS (ESI) m/e 785.1 (M+H)⁺.

### 232.23 tert-butyl 6-[8-(1,3-benzothiazol-2-ylcarbamoyl)-3,4-dihydroisoquinolin-2(1H)-yl]-3-(1-[(3-{2-[(tert-butoxycarbonyl)(methyl)amino]ethoxy)-5,7-dimethyltricyclo[3.3.1.13,7]dec-1-yl)methyl]-5-methyl-1H-pyrazol-4-yl}pyridine-2-carboxylate

To a solution of Example 2.32.22 (10 g) in N,N-dimethylformamide (20 mL) was added benzo[d]thiazol-2-amine (3.24 g), fluoro-N,N,N′,N′-tetramethylformamidinium hexafluorophosphate (5.69 g) and N,N-diisopropylethylamine (5.57 g). The mixture was stirred at 60° C. for 3 hours. The reaction mixture was diluted with ethyl acetate (800 mL) and washed with water and brine, and dried over Na₂SO₄. Filtration and evaporation of the solvent gave a residue that was purified by silica gel chromatography, eluting with 20% ethyl acetate in dichloromethane, to give the title compound. MS (ESI) m/e 915.5 (M+H)⁺.

### 2.32.24 6-[8-(1,3-benzothiazol-2-ylcarbamoyl)-3,4-dihydroisoquinolin-2(1H)-yl]-3-[1-({3,5-dimethyl-7-[2-(methylamino)ethoxyl]tricyclo[3.3.1.13,7]dec-1-yl}methyl)-5-methyl-1H-pyrazol-4-yl]pyridine-2-carboxylic acid

To a solution of Example 2.32.23 (5 g) in dichloromethane (20 mL) was added trifluoroacetic acid (10 mL). The mixture was stirred overnight. The solvent was evaporated under vacuum, and the residue was dissolved in dimethyl sulfoxide/methanol (1:1, 10 mL), and chromatographed via reverse-phase using an Analogix system and a C18 cartridge (300 g), eluting with 10-85% acetonitrile and 0.1% trifluoroacetic acid in water, to give the title compound as a TFA salt. ¹H NMR (300 MHz, dimethyl sulfoxide d₆) δ ppm 12.85 (s, 1H), 8.13-8.30 (m, 2H), 8.03 (d, 1H), 7.79 (d, 1H), 7.62 (d, 1H), 7.32-7.54 (m, 3H), 7.28 (d, 1H), 6.96 (d, 1H), 4.96 (dd, 1H), 3.80-3.92 (m, 4H), 3.48-3.59 (m, 1H), 2.91-3.11 (m, 2H), 2.51-2.59 (m, 4H), 2.03-2.16 (m, 2H), 1.21-1.49 (m, 6H), 0.97-1.20 (m, 4H), 0.87 (s, 6H). MS (ESI) m/e 760.4 (M+H)⁺.

### 2.32.25 3-(1-((3-(2-((((3-(3-aminopropanamido)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid

To a solution of Example 2.32.24 (325 mg) and Example 2.32.7 (382 mg) in N,N-dimethylformamide (9 mL) at 0° C., was added N,N-diisopropylamine (49.1 mg). The reaction mixture was stirred at 0° C. for 5 hours, and acetic acid (22.8 mg) was added. The resulting mixture was diluted with ethyl acetate and washed with water and brine. The organic layer was dried over Na₂SO₄, filtered and concentrated. The residue was dissolved in a mixture of tetrahydrofuran (10 mL) and methanol (5 mL). To this solution at 0° C., was added 1 M aqueous lithium hydroxide solution (3.8 mL). The resulting mixture was stirred at 0° C. for 1 hour, acidified with acetic acid and concentrated.

The concentrate was lyophilized to provide a powder. The powder was dissolved in N,N-dimethylformamide (10 mL), cooled in an ice-bath, and piperidine (1 mL) at 0° C., was added. The mixture was stirred at 0° C. for 15 minutes and 1.5 mL of acetic acid was added. The solution was purified by reverse-phase HPLC using a Gilson system, eluting with 30-80% acetonitrile in water containing 0.1% v/v trifluoroacetic acid, to provide the title compound. MS (ESI) m/e 1172.2 (M+H)⁺.

### 2.32.26 4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)methyl]-2-((N-[6-(2,5-dioxo-2,5-dihydro-1H-pyrrol-1-yl)hexanoyl]-beta-alanyl)amino)phenyl beta-D-glucopyranosiduronic acid

To Example 2.32.25 (200 mg) in N,N-dimethylformamide (5 mL) at 0° C., was added 2,5-dioxopyrrolidin-1-yl 6-(2,5-dioxo-2,5-dihydro-1H-pyrrol-1-yl)hexanoate (105 mg) and N,N-diisopropylethylamine (0.12 mL). The mixture was stirred at 0° C. for 15 minutes, warmed to room temperature and purified by reverse-phase HPLC on a Gilson system using a 100 g C18 column, eluting with 30-80% acetonitrile in water containing 0.1% v/v trifluoroacetic acid, to provide the title compound. ¹H NMR (500 MHz, dimethyl sulfoxide-d₆) δ ppm 12.85 (s, 2H) 9.07 (s, 1H) 8.18 (s, 1H) 8.03 (d, 1H) 7.87 (t, 1H) 7.79 (d, 1H) 7.61 (d, 1H) 7.41-7.53 (m, 3H) 7.36 (q, 2H) 7.28 (s, 1H) 7.03-7.09 (m, 1H) 6.96-7.03 (m, 3H) 6.94 (d, 1H) 4.95 (s, 4H) 4.82 (t, 1H) 3.88 (t, 3H) 3.80 (d, 2H) 3.01 (t, 2H) 2.86 (d, 3H) 2.54 (t, 2H) 2.08 (s, 3H) 2.03 (t, 2H) 1.40-1.53 (m, 4H) 1.34 (d, 2H) 0.90-1.28 (m, 12H) 0.82 (d, 6H). MS (ESI) m/e 1365.3 (M+H)⁺.

### 2.33 Synthesis of Control Synthon 4-[({[2-({3-[(4-{6-[8-(1,3-benzothiazol-2-ylcarbamoyl)-3,4-dihydroisoquinolin-2(l H)-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)methyl]-2-({N-[19-(2,5-dioxo-2,5-dihydro-1H-pyrrol-1-yl)-17-oxo-4,7,10,13-tetraoxa-16-azanonadecan-1-oyl]-beta-alanyl}amino)phenyl beta-D-glucopyranosiduronic acid (Synthon I)

The title compound was prepared using the procedure in Example 2.32.26, replacing 2,5-dioxopyrrolidin-1-yl 6-(2,5-dioxo-2,5-dihydro-1H-pyrrol-1-yl)hexanoate with 2,5-dioxopyrrolidin-1-yl 1-(2,5-dioxo-2,5-dihydro-1H-pyrrol-1-yl)-3-oxo-7,10,13,16-tetraoxa-4-azanonadecan-19-oate. ¹H NMR (500 MHz, dimethyl sulfoxide-d₆) δ ppm 8.95 (s, 1H) 8.16 (s, 1H) 7.99 (d, 1H) 7.57-7.81 (m, 4H) 7.38-7.50 (m, 3H) 7.34 (q, 2H) 7.27 (s, 1H) 7.10 (d, 1H) 7.00 (d, 1H) 6.88-6.95 (m, 2H) 4.97 (d, 4H) 4.76 (d, 2H) 3.89 (t, 2H) 3.84 (d, 2H) 3.80 (s, 2H) 3.57-3.63 (m, 4H) 3.44-3.50 (m, 4H) 3.32-3.43 (m, 6H) 3.29 (t, 2H) 3.16 (q, 2H) 3.02 (t, 2H) 2.87 (s, 3H) 2.52-2.60 (m, 2H) 2.29-2.39 (m, 3H) 2.09 (s, 3H) 1.37 (s, 2H) 1.20-1.29 (m, 4H) 1.06-1.18 (m, 4H) 0.92-1.05 (m, 2H) 0.83 (s, 6H). MS (ESI) m/e 1568.6 (M−H)⁻.

### 2.34 Synthesis of 4-[({[2-({3-[(4-(6-[1-(1,3-benzothiazol-2-ylcarbamoyl)-1,2,3,4-tetrahydroquinolin-7-yl]-2-carboxypyridin-3-yl)-5-methyl-1H-pyrazol-1-yl)methyl]-5,7-dimethyltricyclo[3.3.1.13,7]dec-1-yl}oxy)ethyl](2-methoxyethyl)carbamoyl}oxy)methyl]-3-(2-[2-{N-[6-(2,5-dioxo-2,5-dihydro-1H-pyrrol-1-yl)hexanoyl]-3-sulfo-L-alanyl)amino)ethoxy]ethoxy}phenyl beta-D-glucopyranosiduronic acid (Synthon OG)

### 2.34.1 3-(1-((3-(2-((((2-(2-(2-aminoethoxy)ethoxy)-4-(((2R,3S,4R,5R,6R)-6-carboxy-3,4,5-trihydroxytetrahydro-2H-pyran-2-yl)oxy)benzyl)oxy)carbonyl)(2-methoxyethyl)amino)ethoxy)-5,7-dimethyladamantan-1-yl)methyl)-5-methyl-1H-pyrazol-4-yl)-6-(1-(benzo[d]thiazol-2-ylcarbamoyl)-1,2,3,4-tetrahydroquinolin-7-yl)picolinic acid

To a cold (0° C.) solution of Example 2.11.6 (279 mg) and Example 1.14.9 (240 mg) in N,N-dimethylformamide (10 mL) was added N,N-diisopropylethylamine (0.157 mL). The reaction was slowly warmed to room temperature and was stirred overnight. To the reaction was added water (2 mL) and LiOH H₂O (50 mg), and the mixture was stirred at room temperature for 3 hours. The mixture was acidified with trifluoroacetic acid, filtered and purified by reverse-phase HPLC on a Gilson system (C18 column), eluting with 20-80% acetonitrile in water containing 0.1% trifluoroacetic acid, to provide the title compound. MS (ESI) m/e 1233.0 (M−H)⁻.

### 2.34.2 3-(1-((3-(2-((((2-(2-(2-((R)-2-amino-3-sulfopropanamido)ethoxy)ethoxy)-4-(((2R,3S,4R,5R,6R)-6-carboxy-3,4,5-trihydroxytetrahydro-2H-pyran-2-yl)oxy)benzyl)oxy)carbonyl)(2-methoxyethyl)amino)ethoxy)-5,7-dimethyladamantan-1-yl)methyl)-5-methyl-1H-pyrazol-4-yl)-6-(1-(benzo[d]thiazol-2-ylcarbamoyl)-1,2,3,4-tetrahydroquinolin-7-yl)picolinic acid

To a solution of (R)-2-((((9H-fluoren-9-yl)methoxy)carbonyl)amino)-3-sulfopropanoic acid (45.7 mg) in N,N-dimethylformamide (1 mL) was added O-(7-azabenzotriazol-1-yl)-N,N,N′,N′-tetramethyluronium hexafluorophosphate (45 mg) and N,N-diisopropylethylamine (0.02 mL). The mixture was stirred at room temperature for 10 minutes, and a solution of Example 2.34.1 (96 mg) and N,N-diisopropylethylamine (0.1 mL) in N,N-dimethylformamide (2 mL) was added. The reaction mixture was stirred at room temperature for 3 hours. To the reaction mixture was added diethylamine (0.1 mL), and the reaction was stirred at room temperature overnight. The mixture was diluted with N,N-dimethylformamide (2 mL), filtered and purified by reverse-phase HPLC on a Gilson system (C18 column), eluting with 20-80% acetonitrile in water containing 0.1% trifluoroacetic acid, to provide the title compound. MS (ESI) m/e 1382.2 (M−H)⁻.

### 2.34.3 4-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.13,7]dec-1-yl}oxy)ethyl](2-methoxyethyl)carbamoyl}oxy)methyl]-3-(2-[2-({N-[6-(2,5-dioxo-2,5-dihydro-1H-pyrrol-1-yl)hexanoyl]-3-sulfo-L-alanyl}amino)ethoxy]ethoxy)phenyl beta-D-glucopyranosiduronic acid

The title compound was prepared as described in Example 2.5.3, substituting Example 2.5.2 with Example 2.34.2. ¹H NMR (400 MHz, dimethyl sulfoxide-d₆) δ ppm 8.38 (s, 1H), 7.99 (d, 1H), 7.90-7.70 (m, 6H), 7.44 (s, 1H), 7.35 (t, 1H), 7.28 (d, 1H), 7.24-7.14 (m, 2H), 6.96 (s, 1H), 6.66 (s, 1H), 5.04 (s, 1H), 4.95 (s, 2H), 4.28 (q, 1H), 4.07 (d, 2H), 3.89 (dd, 3H), 3.22 (ddd, 6H), 2.87-2.61 (m, 4H), 2.20 (s, 3H), 2.04 (t, 2H), 1.93 (p, 2H), 1.54-0.90 (m, 20H), 0.83 (d, 7H). MS (ESI) m/e 1575.2 (M−H)⁻.

### 235 Synthesis of 2-[({[2-({3-1(4-6-[5-(1,3-benzothiazol-2-ylcarbamoyl)quinolin-3-)yl-2-carboxypyridin-3-yl)-5-methyl-1H-pyrazol-1-yl)methyl]-5,7-dimethyltricyclo[3.3.1.13,7]dec-1-yl}oxy)ethyl](methyl)carbamoyl}oxy)methyl]-5-{2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid (Synthon OH)

### 2.35.1 3-(1-((3-(2-((((4-(2-(2-aminoethoxy)ethoxy)-2-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(5-(benzo[d]thiazol-2-ylcarbamoyl)quinolin-3-yl)picolinic acid

To a cold (0° C.) solution of Example 2.26.7 (76 mg) and 6-(5-(benzo[d]thiazol-2-ylcarbamoyl)quinolin-3-yl)-3-(1-((3,5-dimethyl-7-(2-(methylamino)ethoxy)adamantan-1-yl)methyl)-5-methyl-1H-pyrazol-4-yl)picolinic acid (62 mg) in N,N-dimethylformamide (2 mL) was added N,N-diisopropylethylamine (0.043 mL). The reaction was slowly warmed to room temperature and stirred overnight. To the reaction was added water (2 mL) and LiOH H₂O (50 mg), and the mixture was stirred at room temperature for 3 hours. The mixture was acidified with trifluoroacetic acid, filtered and purified by reverse-phase HPLC on a Gilson system (C18 column), eluting with 20-80% acetonitrile in water containing 0.1% trifluoroacetic acid, to provide the title compound. MS (ESI) me 1183.3 (M−H)⁻.

### 2.35.2 3-(1-((3-(2-((((4-(2-(2-((R)-2-amino-3-sulfopropanamido)ethoxy)ethoxy)-2-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(5-(benzo[d]thiazol-2-ylcarbamoyl)quinolin-3-yl)picolinic acid

To a solution of (R)-2-((((9H-fluoren-9-yl)methoxy)carbonyl)amino)-3-sulfopropanoic acid (22.3 mg) in N,N-dimethylformamide (1 mL) was added O-(7-azabenzotriazol-1-yl)-N,N,N′,N′-tetramethyluronium hexafluorophosphate (22 mg) and N,N-diisopropylethylamine (0.02 mL). The mixture was stirred at room temperature for 10 minutes, and a solution of Example 2.35.1 (45 mg) and N,N-diisopropylethylamine (0.1 mL) in N,N-dimethylformamide (2 mL) was added. The reaction was stirred at room temperature for 3 hours. To the reaction mixture was added diethylamine (0.1 mL), and the reaction was stirred at room temperature overnight. The mixture was diluted with N,N-dimethylformamide (2 mL), filtered and purified by reverse-phase HPLC on a Gilson system (C18 column), eluting with 20-80% acetonitrile in water containing 0.1% trifluoroacetic acid, to provide the title compound. MS (ESI) m/e 1334.5 (M−H)⁻.

### 2.35.3 2-[({[2-({3-[(4-{6-[5-(1,3-benzothiazol-2-ylcarbamoyl)quinolin-3-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)methyl]-5-{2-[2-{N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid

The title compound was prepared as described in Example 2.34.1, substituting Example 2.5.2 with Example 2.35.2. ¹H NMR (400 MHz, dimethyl sulfoxide-d₆) δ ppm 9.72 (d, 1H), 9.43 (s, 1H), 8.32 (dd, 2H), 8.17 (d, 1H), 8.06 (d, 1H), 8.02-7.92 (m, 2H), 7.86 (d, 1H), 7.82-7.71 (m, 2H), 7.52-7.43 (m, 2H), 7.36 (t, 1H), 7.17 (d, 1H), 6.96 (s, 2H), 6.69 (d, 1H), 6.58 (dd, 1H), 5.03 (dd, 3H), 4.28 (q, 1H), 4.02 (d, 3H), 3.93 (d, 1H), 3.47-3.21 (m, 8H), 3.16 (p, 1H), 2.85 (d, 3H), 2.80-2.63 (m, 2H), 2.22 (s, 3H), 2.04 (t, 2H), 1.53-1.30 (m, 6H), 1.32-0.90 (m, 12H), 0.83 (d, 6H). MS (ESI) m/e 1527.4 (M−H)⁻.

### 2.36 Synthesis of 2-[({[2-({3-[(4-{6-[11-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)methyl]-5-[2-(2-{[6-(2,5-dioxo-2,5-dihydro-1H-pyrrol-1-yl)hexanoyl]amino}ethoxy)ethoxy]phenyl beta-D-glucopyranosiduronic acid (Synthon ON)

### 2.36.1 3-(1-((3-(2-((((4-(2-(2-aminoethoxy)ethoxy)-2-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(1-(benzo[d]thiazol-2-ylcarbamoyl)-1,2,3,4-tetrahydroquinolin-7-yl)picolinic acid, Trifluoroacetic Acid

To a solution of Example 1.1.14 (157 mg) and Example 2.26.7 (167 mg) in N,N-dimethylformamide (3 mL) at 0° C., was added N,N-diisopropylethylamine (188 L). The mixture was warmed to room temperature, stirred overnight and concentrated. The residue was dissolved in methanol (2 mL) and tetrahydrofuran (3 mL). The solution was cooled in an ice water bath and 1M aqueous lithium hydroxide solution (1.14 mL) was added. The mixture was stirred 0° C., at room temperature for 2 hours and concentrated. The residue was dissolved in dimethyl sulfoxide and purified by reverse-phase HPLC on a Gilson system (C18 column), eluting with 20-80% acetonitrile in water containing 0.1% trifluoroacetic acid, to provide the title compound.

### 2.36.2 2-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)methyl]-5-[2-(2-{[6-(2,5-dioxo-2,5-dihydro-1H-pyrrol-1-yl)hexanoyl]amino}ethoxy)ethoxy]phenyl beta-D-glucopyranosiduronic acid

To a solution of Example 2.36.1 (18 mg) and 2,5-dioxopyrrolidin-1-yl 6-(2,5-dioxo-2,5-dihydro-1H-pyrrol-1-yl)hexanoate (6.39 mg) in N,N-dimethylformamide (3 mL) was added N,N-diisopropylethylamine (24 μL). The resulting mixture was stirred for 1 hour and was purified by reverse-phase HPLC on a Gilson system (C18 column), eluting with 20-75% acetonitrile in water containing 0.1% trifluoroacetic acid, to provide the title compound. ¹H NMR (400 MHz, dimethyl sulfoxide-d₆) δ ppm 8.36 (s, 1H), 7.97 (d, 1H), 7.85-7.70 (m, 4H), 7.43 (s, 1H), 7.38-7.30 (m, 1H), 7.26 (d, 1H), 7.23-7.10 (m, 2H), 6.95 (s, 2H), 6.65 (d, 1H), 6.56 (dd, 1H), 5.08-4.94 (m, 3H), 4.02 (dd, 2H), 3.92 (dd, 3H), 3.84 (s, 2H), 3.67 (t, 2H), 3.31-3.20 (m, 2H), 3.16 (q, 2H), 2.91-2.74 (m, 6H), 2.18 (s, 3H), 1.99 (t, 2H), 1.91 (p, 2H), 1.51-1.29 (m, 5H), 1.29-0.88 (m, 9H), 0.81 (d, 6H). MS (ESI) m/e 1380.2 (M−H)⁻.

### 2.37 Synthesis of 4-[({[2-({3-[(4-(6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl)oxy)methyl]-3-{2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid (Synthon OT)

### 2.37.1 3-(1-((3-(2-((((2-(2-(2-aminoethoxy)ethoxy)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic acid

The title compound was prepared by substituting Example 1.6.3 for Example 1.12.10 and Example 2.11.6 for Example 2.26.7 in Example 2.26.8. MS (ESI) m/e 1182.3 (M−H)⁻.

### 2.37.2 3-(1-((3-(2-((((2-(2-(2-((R)-2-amino-3-sulfopropanamido)ethoxy)ethoxy)-4-(((2S,3R,4S,5S,6S)-6-carboxy-3,4,5-trihydroxytetrahydro-2H-pyran-2-yl)oxy)benzyl)oxy)carbonyl)methyl)amino)ethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic acid

The title compound was prepared by substituting Example 2.37.1 for Example 2.9.1 in Example 2.18.1. MS (ESI) m/e 1333.3 (M−H)⁻.

### 2.37.3 4-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)methyl]-3-{2-[2-({N-[6-(2,5-dioxo-2,5-dihydro-1H-pyrrol-1-yl)hexanoyl]-3-sulfo-L-alanyl}amino}ethoxy]ethoxy}phenyl beta-D-glucopyranosiduronic acid

The title compound was prepared by substituting Example 2.37.2 for Example 2.9.1 in Example 2.9.2. ¹H NMR (400 MHz, dimethyl sulfoxide-d₆) δ ppm 9.02 (s, 1H), 8.37 (d, 1H), 8.23 (d, 1H), 8.20 (d, 1H), 8.18 (d, 1H), 8.06 (d, 1H), 8.01 (d, 1H), 7.94 (d, 1H), 7.87 (br d, 1H), 7.81 (d, 1H), 7.77 (br t, 1H), 7.70 (dd, 1H), 7.48 (dd, 1H), 7.48 (s, 1H), 7.37 (dd, 1H), 7.19 (d, 1H), 6.97 (s, 2H), 6.68 (d, 1H), 6.59 (dd, 1H), 5.06 (br m, 1H), 4.97 (s, 2H), 4.31 (m, 1H), 4.09 (m, 2H), 3.90 (m, 5H), 3.71 (m, 2H), 3.45 (m, 5H), 3.36 (m, 3H), 3.28 (m, 4H), 3.19 (m, 2H), 2.82 (br d, 2H), 2.76 (dd, 2H), 2.23 (s, 3H), 2.06 (t, 2H), 1.52-1.32 (m, 6H), 1.32-0.92 (m, 10H), 0.85 (br s, 6H). MS (ESI) m/e 1526.4 (M−H)⁻.

### 2.38 Synthesis of 2-[({[2-({3-[(4-{6-[4-(1,3-benzothiazol-2-ylcarbamoyl)quinolin-6-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)methyl]-5-[2-(2-{[6-(2,5-dioxo-2,5-dihydro-1H-pyrrol-1-yl)hexanoyl]amino}ethoxy)ethoxy]phenyl beta-D-glucopyranosiduronic acid (Synthon OP)

### 2.38.1 3-(1-((3-(2-((((4-(2-(2-aminoethoxy)ethoxy)-2-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(4-(benzo[d]thiazol-2-ylcarbamoyl)quinolin-6-yl)picolinic acid

The title compound was prepared as described in Example 2.36.1, substituting Example 1.1.14 with Example 1.11.4.

### 2.38.2 2-[({[2-({3-[(4-{6-[4-(1,3-benzothiazol-2-ylcarbamoyl)quinolin-6-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)methyl]-5-[2-(2-{[6-(2,5-dioxo-2,5-dihydro-1H-pyrrol-1-yl)hexanoyl]amino}ethoxy)ethoxy]phenyl beta-D-glucopyranosiduronic acid

The title compound was prepared as described in Example 2.36.2, substituting Example 2.36.1 with Example 2.38.1. ¹H NMR (400 MHz, dimethyl sulfoxide-d₆) δ ppm 9.12 (d, 1H), 8.93 (s, 1H), 8.60 (dd, 1H), 8.27 (d, 1H), 8.21 (d, 1H), 8.07 (d, 1H), 7.97-7.90 (m, 2H), 7.81 (d, 2H), 7.47 (d, 2H), 7.37 (t, 1H), 7.17 (d, 1H), 6.96 (s, 2H), 6.67 (d, 1H), 6.58 (dd, 1H), 5.11-4.96 (m, 3H), 4.04 (dd, 2H), 3.92 (d, 1H), 3.86 (s, 2H), 3.40 (q, 5H), 3.34 (t, 2H), 3.31-3.22 (m, 4H), 3.17 (q, 2H), 2.85 (d, 3H), 2.20 (s, 3H), 2.00 (t, 2H), 1.51-1.31 (m, 6H), 1.30-0.88 (m, 13H), 0.82 (d, 6H). MS (ESI) m/e 1400.3 (M+Na)⁺.

### 239 Synthesis of 4-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)methyl]-3-{2-[2-((N-[6-(2,5-dioxo-2,5-dihydro-1H-pyrrol-1-yl)hexanoyl]-3-sulfo-L-alanyl}amino)ethoxy]ethoxy)phenyl beta-D-glucopyranosiduronic acid (Synthon OU)

### 2.39.1 3-(1-((3-(2-((((2-(2-(2-aminoethoxy)ethoxy)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(1-(benzol[d]thiazol-2-ylcarbamoyl)-1,2,3,4-tetrahydroquinolin-7-yl)picolinic acid

The title compound was prepared by substituting Example 1.1.14 for Example 1.12.10 and Example 2.11.6 for Example 2.26.7 in Example 2.26.8. MS (ESI−) m/e 1187.2 (M−H)⁻.

### 2.39.2 3-(1-((3-(2-((((2-(2-(2-((R)-2-amino-3-sulfopropanamido)ethoxy)ethoxy)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(1-(benzo[d]thiazol-2-ylcarbamoyl)-1,2,3,4-tetrahydroquinolin-7-yl)picolinic acid

The title compound was prepared by substituting Example 2.39.1 for Example 2.9.1 in Example 2.18.1. MS (ESI−) m/e 1338.2 (M−H)⁻.

### 239.3 4-[({[2-({3-[(4-{6-[1-(1,3-benzothiazol-2-ylcarbamoyl)-1,2,3,4-tetrahydroquinolin-7-yl]-2-carboxypyridin-3-yl)-5-methyl-1H-pyrazol-1-yl)methyl]-5,7-dimethyltricyclo[3.3.1.13,7]dec-1-yl}oxy)ethyl](methyl)carbamoyloxy)methyl]-3-(2-[2-((N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid

The title compound was prepared by substituting Example 2.39.2 for Example 2.9.1 in Example 2.9.2. ¹H NMR (400 MHz, dimethyl sulfoxide-d₆) δ ppm 8.39 (br s 1H), 8.00 (d, 1H), 7.86 (d, 2H), 7.81 (d, 1H), 7.77 (d, 2H), 7.48 (v br s, 1H), 7.46 (s, 1H), 7.37 (t, 1H), 7.29 (d, 1H), 7.23 (d, 1H), 7.19 (d, 1H), 6.92 (s, 2H), 6.68 (d, 1H), 6.59 (dd, 1H), 5.06 (br m, 1H), 4.97 (s, 2H), 4.31 (m, 1H), 4.09 (m, 2H), 3.96 (br t, 2H), 3.88 (br m, 2H), 3.71 (m, 2H), 3.45 (m, 5H), 3.37 (m, 3H), 3.28 (m, 4H), 3.18 (m, 2H), 2.86 (br m, 5H), 2.75 (dd, 2H), 2.22 (s, 3H), 2.06 (t, 2H), 1.95 (m, 2H), 1.52-1.32 (m, 6H), 1.32-0.92 (m, 12H), 0.85 (br s, 6H). MS (ESI−) m/e 1531.2 (M−H)⁻.

### 2.40 Synthesis of 4-[({[2-({3-[(4-6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl)oxy)methyl]-3-(3-{[6-(2,5-dioxo-2,5-dihydro-1H-pyrrol-1-yl)hexanoyl]amino}propoxy)phenyl beta-D-glucopyranosiduronic acid (Synthon 00)

### 2.40.1 3-(1-((3-(2-((((2-(3-aminopropoxy)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(1-(benzo[d]thiazol-2-ylcarbamoyl)-1,2,34-tetrahydroquinolin-7-yl)picolinic acid

The title compound was prepared as described in Example 2.36.1, substituting Example 2.26.7 with Example 2.28.3. MS (ESI) m/e 1159.2 (M+H)⁺.

### 2.40.2 4-[({[2-({3-[(4-{6-([1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)methyl]-3-(3-{[6-(2,5-dioxo-2,5-dihydro-1H-pyrrol-1-yl)hexanoyl]amino}propoxy)phenyl beta-D-glucopyranosiduronic acid

The title compound was prepared as described in Example 2.36.2, substituting Example 2.36.1 with Example 2.40.1. ¹H NMR (400 MHz, dimethyl sulfoxide-d₆) δ ppm 8.38 (s, 1H), 7.98 (d, 1H), 7.87-7.72 (m, 2H), 7.44 (s, 1H), 7.35 (t, 1H), 7.28 (d, 1H), 7.19 (dd, 2H), 6.96 (s, 2H), 6.62 (d, 1H), 6.57 (dd, 1H), 5.03 (s, 1H), 4.95 (s, 2H), 4.03-3.81 (m, 8H), 3.42-3.20 (m, 7H), 3.16 (q, 2H), 2.90-2.75 (m, 5H), 2.20 (s, 3H), 2.01 (t, 2H), 1.97-1.87 (m, 2H), 1.80 (t, 2H), 1.45 (td, 4H), 1.13 (d, 8H), 0.83 (d, 6H). MS (ESI) m/e 1350.2 (M−H)⁻.

### 2.41 Synthesis of 4-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl)oxy)methyl]-3-[3-(N-[6-(2,5-dioxo-2,5-dihydro-1H-pyrrol-1-yl)hexanoyl]-3-sulfo-L-alanyl}amino)propoxy]phenyl beta-D-glucopyranosiduronic acid (Synthon OQ)

### 2.41.1 3-(1-((3-(2-((((2-(3-((R)-2-amino-3-sulfopropanamido)propoxy)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(1-(benzo[d]thiazol-2-ylcarbamoyl)-1,2,3,4-tetrahydroquinolin-7-yl)picolinic acid

To a solution of (R)-2-((((9H-fluoren-9-yl)methoxy)carbonyl)amino)-3-sulfopropanoic acid (35.4 mg) and O-(7-azabenzotriazol-1-yl)-N,N,N′,N′-tetramethyluronium hexafluorophosphate (29.8 mg) in N,N-dimethylformamide (1 mL) at 0° C., was added N,N-diisopropylethylamine (30 μL). The resulting mixture was stirred for 15 minutes and added to a mixture of Example 2.40.1 (70 mg) and N,N-diisopropylethylamine (80 μL) in N,N-dimethylformamide (2 mL). The resulting mixture was stirred for 1 hour. Diethylamine (62.2 μL) was added, and the mixture was stirred for 1 hour. The reaction was cooled in ice-bath and trifluoroacetic acid (93 μL) was added. The mixture was diluted with dimethyl sulfoxide (5.5 mL) and purified by reverse-phase HPLC on a Gilson system (C18 column), eluting with 20-75% acetonitrile in water containing 0.1% trifluoroacetic acid, to provide the title compound.

### 2.41.2 4-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)methyl]-3-[3-(N-[6-(2,5-dioxo-2,5-dihydro-1H-pyrrol-1-yl)hexanoyl]-3-sulfo-L-alanylamino)propoxy]phenyl beta-D-glucopyranosiduronic acid

The title compound was prepared as described in Example 2.36.2, substituting Example 2.36.1 with Example 2.41.1. ¹H NMR (501 MHz, dimethyl sulfoxide-d₆) δ ppm 8.37 (s, 1H), 7.98 (d, 1H), 7.87-7.72 (m, 5H), 7.44 (s, 1H), 7.35 (t, 1H), 7.27 (d, 1H), 7.20 (t, 1H), 7.16 (d, 1H), 6.96 (s, 2H), 6.63 (d, 1H), 6.55 (dd, 1H), 5.02 (s, 1H), 4.95 (s, 2H), 4.26 (q, 1H), 4.04-3.79 (m, 8H), 3.32-3.08 (m, 4H), 2.89-2.66 (m, 7H), 2.35 (q. OH), 2.20 (s, 3H), 2.03 (t, 2H), 1.93 (p, 2H), 1.80 (t, 2H), 1.52-1.30 (m, 6H), 1.30-0.89 (m, 13H), 0.83 (d, 6H). MS (ESI) m/e 1502.2 (M−H)⁻.

### 2.42 Synthesis of 2-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl}methyl]-5,7-dimethyltricyclo[3.3.1.13,7]dec-1-yloxy)ethyl](methyl)carbamoyl)oxy)methyl]-5-12-[2-((N-[6-(2,5-dioxo-2,5-dihydro-1H-pyrrol-1-yl)hexanoyl]-3-sulfo-L-alanyl}amino)ethoxy]ethoxy)phenyl beta-D-glucopyranosiduronic acid (Synthon OR)

### 2.42.1 3(1-((3-(2-((((4-(2-(2-((R)-2-amino-3-sulfopropanamido)ethoxy)ethoxy)-2-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(1-(benzo[d]thiazol-2-ylcarbamoyl)-1,2,3,4-tetrahydroquinolin-7-yl)picolinic acid

The title compound was prepared as described in Example 2.41.1, substituting Example 2.40.1 with Example 2.36.1. MS (ESI) m/e 1338.2 (M−H)⁻.

### 2.42.2 2-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)methyl]-5-{2-[2-((N-[6-(2,5-dioxo-2,5-dihydro-1H-pyrrol-1-yl)hexanoyl]-3-sulfo-L-alanyl)amino)ethoxy]ethoxy}phenyl beta-D-glucopyranosiduronic acid

The title compound was prepared as described in Example 2.36.2, substituting Example 2.36.1 with Example 2.42.1. ¹H NMR (500 MHz, dimethyl sulfoxide-d₆) δ ppm 8.39 (s, 1H), 8.00 (d, 1H), 7.86 (t, 2H), 7.83-7.73 (m, 3H), 7.45 (s, 1H), 7.40-7.32 (m, 1H), 7.29 (d, 1H), 7.26-7.13 (m, 2H), 6.97 (s, 2H), 6.70 (d, 1H), 6.59 (dd, 1H), 5.11-4.94 (m, 3H), 4.29 (dt, 1H), 4.04 (dd, 2H), 3.99-3.91 (m, 3H), 3.87 (d, 2H), 3.69 (t, 2H), 3.40-3.07 (m, 7H), 2.91-2.74 (m, 6H), 2.69 (dd, 1H), 2.21 (s, 3H), 2.05 (t, 2H), 1.94 (p, 2H), 1.53-1.32 (m, 5H), 1.31-0.90 (m, 7H), 0.84 (d, 6H). MS (ESI) m/e 1531.2 (M−H)⁻.

### 2.43 Synthesis of 4-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.13,7]dec-1-yl}oxy)ethyl](2-methoxyethyl)carbamoyl}oxy)methyl]-3-{2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid (Synthon OS)

### 2.43.1 3-(1-((3-(2-((((2-(2-(2-aminoethoxy)ethoxy)-4-(((2S,3R,4S,5S,6S-6-carboxy-3,4,5-trihydroxytetrahydro-2H-pyran-2-yl)oxy)benzyl)oxy)carbonyl(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic acid

The title compound was prepared as described in Example 2.34.1, substituting Example 2.5.2 with Example 1.15.1. MS (ESI) m/e 1228.1 (M−H)⁻.

### 2.43.2 3-(1-((3-(2-((((2-(2-(2-((R)-2-amino-3-sulfopropanamido)ethoxy)-4-(((2S,3R,4S,5S,6S)-6-carboxy-3,4,5-trihydroxytetrahydro-2H-pyran-2-yl)oxy)benzyl)oxy)carbonyl)(2-methyl)amino)ethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic acid

The title compound was prepared as described in Example 2.34.2, substituting Example 2.34.1 with Example 2.43.2. MS (ESI) m/e 1379.1.1 (M+H)⁺.

### 2.43.3 4-[({[2-({3-[(4-6-[8-(1,3-benzothiazol-2-ylcarbamoyl)naphthalen-2-yl]-2-carboxypyridin-3-yl)-5-methyl-1H-pyrazol-1-yl)methyl]-5,7-dimethyltricyclo[3.3.1.13,7]dec-1-yl}oxy)ethyl](2-methoxyethyl)carbamoyl)oxy)methyl]-3-(2-[2-((N-[6-(2,5-dioxo-2,5-dihydro-1H-pyrrol-1-yl)hexanoyl]-3-sulfo-L-alanyl}amino)ethoxy]ethoxy)phenyl beta-D-glucopyranosiduronic acid

The title compound was prepared as described in Example 2.34, substituting Example 2.34.2 with Example 2.43.2. ¹H NMR (400 MHz, dimethyl sulfoxide-d₆) δ ppm 9.00 (s, 1H), 8.36 (d, 1H), 8.27-8.12 (m, 3H), 8.05 (d, 1H), 8.00 (d, 1H), 7.92 (d, 1H), 7.85 (d, 1H), 7.79 (d, 1H), 7.75 (t, 1H), 7.69 (t, 1H), 7.52-7.43 (m, 2H), 7.35 (t, 1H), 7.24-7.12 (m, 1H), 6.95 (s, 2H), 6.66 (s, 1H), 6.57 (d, 1H), 5.04 (d, 1H), 4.95 (s, 2H), 4.29 (q, 1H), 4.15-4.01 (m, 2H), 3.86 (d, 3H), 3.46-3.11 (m, 16H), 2.84-2.62 (m, 2H), 2.21 (d, 3H), 2.04 (t, 2H), 1.53-1.30 (m, 6H), 1.28-0.89 (m, 6H), 0.82 (d, 7H). MS (ESI) m/e 1570.4 (M−H)⁻.

### 2.44 Synthesis of N-[6-(2,5-dioxo-2,5-dihydro-1H-pyrrol-1-yl)hexanoyl]-L-valyl-N-[4-({[{2-[{8-(1,3-benzothiazol-2-ylcarbamoyl)-2-[6-carboxy-5(1-{[3-(2-methoxyethoxy)-5,7-dimethyltricyclo[3.3.1.13,7]dec-1-yl]methyl}-5-methyl-1H-pyrazol-4-yl)pyridin-2-yl]-1,2,3,4-tetrahydroisoquinolin-6-yl}(methyl)amino]ethyl}(methyl)carbamoyl]oxy}methyl)phenyl]-L-alaninamide (Synthon OX)

The title compound was prepared as described in Example 2.30, substituting Example 1.17.10 with Example 1.21.12. MS (ESI) m/e 1359.5 (M+H)⁺, 1357.5 (M−H)⁻.

### 2.45 Synthesis of N-[6-(2,5-dioxo-2,5-dihydro-1H-pyrrol-1-yl)hexanoyl]-L-valyl-N-({4-[({[2-((3-[(4-6-[8-(1,3-benzothiazol-2-ylcarbamoyl)-6-methoxy-3,4-dihydroisoquinolin-2(1H)-yl]-2-carboxypyridin-3-yl-5-methyl-1H-pyrazol-1-yl)methyl]-5,7-dimethyltricyclo[3.3.1.13,7]dec-1-yl}oxy)ethyl](methyl)carbamoyl}oxy)methyl]phenyl)-L-alaninamide (Synthon OZ)

The title compound was prepared as described in Example 2.30, substituting Example 1.17.10 with Example 1.22.9. MS (ESI) m/e 1302.5 (M+H)⁺, 1300.5 (M−H)⁻.

### 2.46 Synthesis of 2-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.13,7]dec-1-yl}oxy)ethyl](2-methoxyethyl)carbamoyl}oxy)methyl]-5-{2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid (Synthon PA)

### 2.46.1 3-(1-((3-(2-((((4-(2-(2-aminoethoxy)ethoxy)-2-(((2S,3R,4S,5S,6S)-6-carboxy-3,4,5-trihydroxytetrahydro-2H-pyran-2-yl)oxy)benzyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic acid

The title compound was prepared as described in Example 2.43.1, substituting Example 2.11.6 with Example 2.26.7. MS (ESI) m/e 1228.1 (M−H)⁻.

### 2.46.2 3-(1-((3-(2-((((4-(2-(2-((R)-2-amino-3-sulfopropanamido)ethoxy)ethoxy(((2S,3R,4S,5S,6S)-6-carboxy-3,4,5-trihydroxytetrahydro-2H-pyran-2-yl)oxy)benzyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic acid

The title compound was prepared as described in Example 2.34.2, substituting Example 2.34.1 with Example 2.46.1. MS (ESI) m/e 1377.5 (M−H)⁻.

### 2.46.3 2-[({[2-({3-[(4-{6-[8-(1,3-benzothiazol-2-ylcarbainoyl)naphthalen-2-yl]-2-carboxypyridin-3-yl}-5-methyl-1H-pyrazol-1-yl)methyl]-5,7-dimethyltricyclo[3.3.1.13,7]dec-1-yl}oxy)ethyl](2-methoxyethyl)carbamoyl}oxy)methyl]-5-{2-[2-({N-[6-(2,5-dioxo-2,5-dihydro-1H-pyrrol-1-yl)hexanoyl]-3-sulfo-L-alanyl}amino)ethoxy]ethoxy}phenyl beta-D-glucopyranosiduronic acid

The title compound was prepared as described in Example 2.34, substituting Example 2.34.2 with Example 2.46.2. ¹H NMR (501 MHz, dimethyl sulfoxide-d₆) δ ppm 13.08 (s, 1H), 9.00 (s, 1H), 8.36 (d, 1H), 8.25-8.12 (m, 3H), 8.05 (d, 1H), 8.00 (d, 1H), 7.92 (d, 1H), 7.85 (d, 1H), 7.78 (dd, 2H), 7.72-7.65 (m, 1H), 7.50-7.43 (m, 2H), 7.35 (t, 1H), 7.21-7.14 (m, 1H), 6.96 (s, 2H), 6.69 (d, 1H), 6.58 (d, 1H), 5.13-4.93 (m, 3H), 4.28 (q, 1H), 4.03 (dd, 2H), 3.94 (d, 1H), 3.86 (d, 2H), 3.67 (t, 2H), 3.31-3.08 (m, 8H), 2.83-2.64 (m, 2H), 2.21 (d, 3H), 2.04 (t, 2H), 1.53-1.30 (m, 5H), 1.30-0.89 (m, 11H), 0.89-0.75 (m, 6H). MS (ESI) m/e 1570.5 (M−H)⁻.

### 2.47 Synthesis of 2-[({[2-({3-[(4-6-[5-(1,3-benzothiazol-2-ylcarbamoyl)quinolin-3-yl]-2-carboxypyridin-3-yl)-5-methyl-1H-pyrazol-1-yl)methyl]-5,7-dimethyltricyclo[3.3.1.13,7]dec-1-yl}oxy)ethyl](methyl)carbamoyl}oxy)methyl]-5-[2-(2-{[6-(2,5-dioxo-2,5-dihydro-1H-pyrrol-1-yl)hexanoyl]amino}ethoxy)ethoxy]phenyl beta-D-glucopyranosiduronic acid (Synthon QL)

### 2.47.1 3-(1-((3-(2-((((4-(2-(2-aminoethoxy)ethoxy)-2-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(5-(benzo[d]thiazol-2-ylcarbamoyl)quinolin-3-yl)picolinic acid

The title compound was prepared as described in Example 2.36.1, substituting Example 1.1.14 with Example 1.10.3.

### 2.47.2 2-[({[2-({3-[(4-6-[5-(1,3-benzothiazol-2-ylcarbamoyl)quinolin-3-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl)oxy)methyl]-5-[2-(2-{[6-(2,5-dioxo-2,5-dihydro-1H-pyrrol-1-yl)hexanoyl]amino}ethoxy)ethoxy]phenyl beta-D-glucopyranosiduronic acid

The title compound was prepared as described in Example 2.36., substituting Example 2.36.1 with Example 2.47.1. ¹H NMR (501 MHz, dimethyl sulfoxide-d₆) δ ppm 13.17 (s, 1H), 9.70 (d, 1H), 9.39 (s, 1H), 8.31 (dd, 2H), 8.16 (d, 1H), 8.06 (dd, 1H), 8.01-7.90 (m, 2H), 7.83-7.71 (m, 2H), 7.52-7.43 (m, 2H), 7.39-7.31 (m, 1H), 7.18 (d, 1H), 6.96 (s, 2H), 6.65 (d, 1H), 6.58 (dd, 1H), 5.04 (s, 1H), 4.96 (s, 2H), 4.09 (dtd, 2H), 3.87 (s, 2H), 3.70 (t, 2H), 3.40-3.14 (m, 7H), 2.85 (d, 3H), 2.22 (s, 3H), 2.01 (t, 2H), 1.49-1.30 (m, 6H), 1.30-0.90 (m, 10H), 0.90-0.74 (m, 6H). MS (ESI) m/e 1400.4 (M+Na)⁺.

### 2.48 Synthesis of 4-[({[2-((3-[(4-6-[5(1,3-benzothiazol-2-ylcarbamoyl)quinolin-3-yl]-2-carboxypyridin-3-yl{-5-methyl-1H-pyrazol-1-yl)methyl]-5,7-dimethyltricyclo[3.3.1.13,7]dec-1-yl}oxy)ethyl](methyl)carbamoyl}oxy)methyl]-3-[2-(2-{[6-(2,5-dioxo-2,5-dihydro-1H-pyrrol-1-yl)hexanoyl]amino}ethoxy)ethoxy]phenyl beta-D-glucopyranosiduronic acid (Synthon QM)

### 2.48.1 3-(1-((3-(2-((((2-(2-(2-aminoethoxy)ethoxy)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(5-(benzo[d]thiazol-2-ylcarbamoyl)quinolin-3-yl)picolinic acid

To a solution of Example 1.10.3 (208 mg) and Example 2.11.6 (267 mg) in N,N-dimethylformamide (2 mL) at 0° C., was added N,N-diisopropylethylamine (251 L). The resulting mixture was stirred at room temperature overnight and concentrated. The residue was dissolved in methanol (3 mL) and tetrahydrofuran (5 mL). The solution was cooled in an ice water bath and 1M aqueous lithium hydroxide solution was added (2.87 mL). The mixture was stirred at 0° C. for 2 hours and was acidified with trifluoroacetic acid. The reaction mixture was concentrated under reduced pressure. The residue was diluted with dimethyl sulfoxide and purified by reverse-phase HPLC on a Gilson system (C18 column), eluting with 20-75% acetonitrile in water containing 0.1% trifluoroacetic acid, to provide the title compound. MS (ESI) m/e 1185.1 (M+H)⁺.

### 2.48.2 4-[({[2-({3-[(4-{6-[5-(1,3-benzothiazol-2-ylcarbamoyl)quinolin-3-yl]-2-carboxypyridin-3-yl}-5-methyl-1H-pyrazol-1-yl)methyl]-5,7-dimethyltricyclo[3.3.1.13,7]dec-1-yl}oxy)ethyl(methyl]carbamoyl}oxy)methyl]-3-[2-(2-{[6-(2,5-dioxo-2,5-dihydro-1H-pyrrol-1-yl)hexanoyl]amino}ethoxy)ethoxy]phenyl beta-D-glucopyranosiduronic acid

The title compound was prepared as described in Example 2.36.2, substituting Example 2.36.1 with Example 2.48.1. ¹H NMR (400 MHz, dimethyl sulfoxide-d₆) δ ppm 13.18 (s, 1H), 9.70 (d, 1H), 9.39 (s, 1H), 8.31 (dd, 2H), 8.16 (d, 1H), 8.06 (d, 1H), 8.01-7.90 (m, 2H), 7.80 (d, 2H), 7.52-7.43 (m, 2H), 7.39-7.32 (m, 1H), 7.18 (d, 1H), 6.96 (s, 2H), 6.67 (d, 1H), 6.58 (dd, 1H), 5.11-4.90 (m, 3H), 4.03 (d, 2H), 3.95-3.82 (m, 3H), 3.68 (t, 2H), 3.48-3.23 (m, 10H), 3.18 (t, 2H), 2.85 (d, 3H), 2.22 (s, 3), 2.00 (t, 2H), 1.51-1.31 (m, 5H), 1.19 (dd, 10H), 0.83 (d, 6H). MS (ESI) m/e 1376.4 (M−H)⁻.

### 2.49 Synthesis of 6-[5-(1,3-benzothiazol-2-ylcarbamoyl)quinolin-3-yl]-3-(1-{[3-(2-{[6-(2,5-dioxo-2,5-dihydro-1H-pyrrol-1-yl)hexanoyl](methyl)amino}ethoxy)-5,7-dimethyltricyclo[3.3.1.13,7]dec-1-yl]methyl}-5-methyl-1H-pyrazol-4-yl)pyridine-2-carboxylic acid (Synthon QN)

The title compound was prepared as described in Example 2.36.2, substituting Example 2.36.1 with Example 1.10.3. ¹H NMR (400 MHz, dimethyl sulfoxide-d₆) δ ppm 13.21 (s, 1H), 9.70 (d, 1H), 9.40 (s, 1H), 8.42-8.27 (m, 2H), 8.16 (d, 1H), 8.06 (d, 1H), 8.04-7.90 (m, 2H), 7.80 (d, 1H), 7.56-7.44 (m, 2H), 7.42-7.31 (m, 1H), 6.95 (d, 2H), 3.87 (s, 2H), 3.55-3.18 (m, 5H), 2.95 (s, 1H), 2.76 (s, 2H), 2.28 (t, 1H), 2.22 (s, 4H), 1.53-1.29 (m, 6H), 1.28-0.91 (m, 10H), 0.84 (s, 6H). MS (ESI) m/e 949.1 (M+H)⁺.

### 2.50 Synthesis of 4-[({[2-({3-[(4-{6-[7-(1,3-benzothiazol-2-ylcarbamoyl)-1H-indol-2-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)methyl]-2-({N-[3-(2,5-dioxo-2,5-dihydro-1H-pyrrol-1-yl)propanoyl]-beta-alanyl}amino)phenyl beta-D-glucopyranosiduronic acid (Synthon QT)

### 2.50.1 3-(1-((3-(2-((((3-(3-aminopropanamido)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(7-(benzo[d]thiazol-2-ylcarbamoyl)-1H-indol-2-yl)picolinic acid

The title compound was prepared by substituting Example 1.27.4 for Example 2.32.24 in Example 2.32.25. MS (ESI) m/e: 1156.6 (M+H)⁺.

### 2.50.2 4-[({[2-({3-[(4-{6-[7-(1,3-benzothiazol-2-ylcarbamoyl)-1H-indol-2-yl]-2-carboxypyridin-3-yl}-5-methyl-1H-pyrazol-1-yl)methyl]-5,7-dimethytricyclo[3.3.1.13,7]dec-1-yl}oxy)ethyl](methyl)carbamoyl}oxy)methyl]-2-((N-[3-(2,5-dioxo-2,5-dihydro-1H-pyrrol-1-yl)propanoyl]-beta-alanyl)amino)phenyl beta-D-glucopyranosiduronic acid

The title compound was prepared by substituting Example 2.50.1 for Example 2.11.7 in Example 2.11.8. ¹H NMR (501 MHz, dimethyl sulfoxide-d₆) δ ppm 13.00 (s, 2H): 9.06 (s, 1H), 8.29 (dd, 1H), 8.22 (d, 1H), 8.18 (s, 1H), 8.04 (t, 2H), 7.97 (d, 1H), 7.90 (d, 1H), 7.79 (d, 1H), 7.50-7.43 (m, 3H), 7.35 (ddd, 1H), 7.25 (t, 1H), 7.06 (d, 1H), 7.01 (dd, 1H), 6.94 (s, 2H), 4.96 (s, 2H), 4.81 (s, 1H), 3.33-3.25 (m, 6H), 2.87 (d, 3H), 2.50 (d, 3H), 2.31 (dd, 2H), 2.21 (s, 3H), 1.38 (d, 2H), 1.30-0.77 (m, 18H). MS (ESI) m e 1305.2 (M−H)⁻.

### 2.51 Synthesis of 4-[({[2-({3-[(4-(6-[7-(1,3-benzothiazol-2-ylcarbamoyl)-1H-indol-2-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl)oxy)methyl]-3-[2-(2-{[3-(2,5-dioxo-2,5-dihydro-1H-pyrrol-1-yl)propanoyl]amino}ethoxy)ethoxy]phenyl beta-D-glucopyranosiduronic acid (Synthon RF)

### 2.51.1 3-(1-((3-(2-((((2-(2-(2-aminoethoxy)ethoxy)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(7-(benzo[d]thiazol-2-ylcarbamoyl)-1H-indol-2-yl)picolinic acid

The title compound was prepared by substituting Example 1.27.4 for Example 1.12.10 in Example 2.11.7. MS (ESI) m/e: 1172.9 (M+H)⁺.

### 2.51.2 4-[({[2-({3-[(4-{6-[7-(1,3-benzothiazol-2-ylcarbamoyl)-1H-indol-2-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)methyl]-3-[2-(2-{[3-(2,5-dioxo-2,5-dihydro-1H-pyrrol-1-yl)propanoyl]amino}ethoxy)ethoxy]phenyl beta-D-glucopyranosiduronic acid

The title compound was prepared by substituting Example 2.51.1 for Example 2.11.7 in Example 2.11.8. ¹H NMR (501 MHz, dimethyl sulfoxide-d₆) δ ppm 11.16 (s, 2H), 8.27 (d, 1H), 8.19 (d, 1H), 8.06-7.94 (m, 3H), 7.88 (d, 1H), 7.77 (d, 1H), 7.50-7.39 (m, 3H), 7.33 (t, 1H), 7.26-7.13 (m, 2H), 6.93 (s, 2H), 6.63 (d, 1H), 6.57 (dd, 1H), 5.03 (d, 1H), 4.94 (s, 2H), 4.13-4.00 (m, 2H), 3.86 (d, 3H), 3.14 (q, 2H), 2.83 (d, 3H), 2.29 (t, 2H), 2.20 (s, 3H), 1.36 (d, 2H), 1.28-0.73 (m, 16H). MS (ESI) mile 1322.4 (M−H)⁻.

### 2.52 Synthesis of 4-[({[2-({3-[(4-{6-[7-(1,3-benzothiazol-2-ylcarbamoyl)-1H-indol-2-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)methyl]-3-{2-[2-({N-[3-(2,5-dioxo-2,5-dihydro-1H-pyrrol-1-yl)propanoyl]-3-sulfo-L-alanyl}amino)ethoxy]ethoxy}phenyl beta-D-glucopyranosiduronic acid (Synthon RG)

### 2.52.1 3-(1-((3-(2-((((2-(2-(2-((R)-2-amino-3-sulfopropanamido)ethoxy)ethoxy)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(7-(benzo[d]thiazol-2-ylcarbamoyl)-1H-indol-2-yl)picolinic acid

The title compound was prepared by substituting Example 2.51.1 for Example 2.9.1 in Example 2.18.1. MS (ESI) m/e:1325.5 (M+H)⁺.

### 2.52.2 4-[({[2-({3-1(4-(6-[7-(1,3-benzothiazol-2-ylcarbamoyl)-1H-indol-2-yl]-2-carboxypyridin-3-yl}-5-methyl-1H-pyrazol-1-yl)methyl]-5,7-dimethyltricyclo[3.3.1.13,7]dec-1-yl}oxy)ethyl(methyl)carbamoyl)oxy)methyl]-3-{2-[2-((N-[3-(2,5-dioxo-2,5-dihydro-1H-pyrrol-1-yl)propanoyl]-3-sulfo-L-alanyl}amino)ethoxy]ethoxy)phenyl beta-D-glucopyranosiduronic acid

The title compound was prepared by substituting Example 2.52.1 for Example 2.11.7 in Example 2.11.8. ¹H NMR (501 MHz, dimethyl sulfoxide-d₆) δ ppm 11.17 (s, 2H), 8.27 (d, 1H), 8.20 (d, 1H), 8.03 (dd, 2H), 7.96 (d, 1H), 7.89 (d, 1H), 7.82-7.75 (m, 2H), 7.50 (s, 1H), 7.48-7.41 (m, 2H), 7.34 (t, 1H), 7.24 (t, 1H), 7.18 (d, 1H), 6.93 (s, 2H), 6.66 (d, 1H), 6.58 (dd, 1H), 5.04 (d, 1H), 4.95 (s, 2H), 3.70 (t, 2H), 3.58 (t, 2H), 3.48-3.14 (m, 11H), 2.89-2.79 (m, 4H), 2.73 (dd, 1H), 2.37 (m, 2H), 2.21 (s, 3H), 1.45-0.73 (m, 19H). MS (ESI) m/e 1473.3 (M−H)⁻.

### 2.53 Synthesis of 4-[({[2-({3-[(4-6-[7-(1,3-benzothiazol-2-ylcarbamoyl)-3-methyl-1H-indol-2-yl]-2-carboxypyridin-3-yl)-5-methyl-1H-pyrazol-1-yl)methyl]-5,7-dimethyltricyclo[3.3.1.13,7]dec-1-yl}oxy)ethyl](methyl)carbamoyl}oxy)methyl]-3-[2-(2-{[3-(2,5-dioxo-2,5-dihydro-1H-pyrrol-1-yl)propanoyl]amino}ethoxy)ethoxy]phenyl beta-D-glucopyranosiduronic acid (Synthon SF)

### 2.53.1 3-(1-((3-(2-((((2-(2-(2-aminoethoxy)ethoxy)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(7-(benzo[d]thiazol-2-ylcarbamoyl)-3-methyl-1H-indol-2-yl)picolinic acid

The title compound was prepared by substituting Example 1.29.7 for Example 1.12.10 in Example 2.11.7. MS (ESI) m/e:1187.1 (M+H)⁺.

### 2.53.2 4-[([2-({3-[(4-{6-[7-(1,3-benzothiazol-2-ylcarbamoyl)-3-methyl-1H-indol-2-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl)oxy)methyl]-3-[2-(2-{[3-(2,5-dioxo-2,5-dihydro-1H-pyrrol-1-yl)propanoyl]amino}ethoxy)ethoxy]phenyl beta-D-glucopyranosiduronic acid

The title compound was prepared by substituting Example 2.53.1 for Example 2.11.7 in Example 2.11.8. ¹H NMR (501 MHz, dimethyl sulfoxide-d₆) δ ppm 11.01 (s, 1H), 8.28 (d, 1H), 8.06-7.94 (m, 4H), 7.91 (d, 1H), 7.76 (d, 1H), 7.50-7.42 (m, 2H), 7.32 (td, 1H), 7.26-7.15 (m, 2H), 6.93 (s, 2H), 6.64 (d, 1H), 6.58 (dd, 1H), 5.03 (d, 1H), 4.95 (s, 2H), 4.11-3.99 (m, 2H), 3.87 (d, 3H), 3.68 (t, 2H), 3.56 (dd, 2H), 3.47-3.33 (m, 5H), 3.33-3.19 (m, 4H), 3.14 (q, 2H), 2.84 (d, 3H), 2.63 (s, 3H), 2.30 (dd, 2H), 2.21 (s, 3H), 1.42-0.72 (m, 21H). MS (ESI) m/e 1336.3 (M−H)⁻.

### 2.54 Synthesis of N-[6-(2,5-dioxo-2,5-dihydro-1H-pyrrol-1-yl)hexanoyl]-L-valyl-N-{4-[({[2-{3-[(4-{6-[4-(1,3-benzothiazol-2-ylcarbamoyl)isoquinolin-6-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)methyl]phenyl}-N5-carbamoyl-L-ornithinamide (Synthon SR)

The title compound was prepared as described in Example 2.2, substituting Example 1.3.2 with Example 1.26.10. ¹H NMR (400 MHz, dimethyl sulfoxide-d₆) δ ppm 13.28 (s, 2H), 9.96 (s, 1H), 9.59 (s, 1H), 9.03 (d, 2H), 8.53 (d, 1H), 8.42 (d, 1H), 8.25 (d, 1H), 8.05 (t, 2H), 7.97 (d, 1H), 7.78 (dd, 2H), 7.58 (d, 2H), 7.47 (d, 2H), 7.36 (t, 1H), 7.26 (d, 2H), 6.97 (s, 2H), 5.96 (s, 1H), 4.96 (s, 2H), 4.45-4.29 (m, 1H), 4.17 (t, 1H), 3.51-3.18 (m, 6H), 3.07-2.75 (m, 4H), 2.22 (s, 3H), 2.11 (dq, 1H), 2.02-1.82 (m, 1H), 1.76-0.88 (m, 18H), 0.81 (dd, 14H). MS (ESI) m/e 1352.4 (M−H)⁻.

### 2.55 Synthesis of 4-[({[2-((3-[(4-6-[1-(1,3-benzothiazol-2-ylcarbamoyl)-5,6-dihydroimidazo[1,5-a]pyrazin-7(8H)-yl]-2-carboxypyridin-3-yl)-5-methyl-1H-pyrazol-1-yl)methyl]-5,7-dimethyltricyclo[3.3.1.13,7]dec-1-yl}oxy)ethyl]carbamoyl)oxy)methyl]-3-[2-(2-[(2,5-dioxo-2,5-dihydro-1H-pyrrol-1-yl)acetyl]amino)ethoxy)e)ethoxy]phenyl beta-D-glucopyranosiduronic acid (Synthon YZ)

### 2.55.1 3-(1-((3-(2-((((2-(2-(2-aminoethoxy)ethoxy)-4-(((2S,3R,4S,5S,6S)-6-carboxy-3,4,5-trihydroxytetrahydro-2H-pyran-2-yl)oxy)benzyl)oxy)carbonyl)amino)ethoxy)-5,7-dimethyladamantan-1-yl)methyl)-5-methyl-1H-pyrazol-4-yl)-6-(1-(benzo[d]thiazol-2-ylcarbamoyl)-5,6-dihydroimidazo[1,5-a]pyrazin-7(8H)-yl)picolinic acid

The title compound was prepared by substituting Example 1.4.10 for Example 1.12.10 in Example 2.11.7. MS (ESI) m/e 1165 (M+H)⁺, 1163 (M−H)⁻.

### 2.55.2 4-[({[2-({3-[(4-(6-[1-(1,3-benzothiazol-2-ylcarbamoyl)-5,6-dihydroimidazo[1,5-a]pyrazin-7(8H)-yl]-2-carboxypyridin-3-yl)-5-methyl-1H-pyrazol-1-yl)methyl]-5,7-dimethyltricyclo[3.3.1.13,7]dec-1-yl}oxy)ethyl]carbamoyl}oxy)methyl]-3-[2-(2-{[(2,5-dioxo-2,5-dihydro-1H-pyrrol-1-yl)acetyl]amino}ethoxy)ethoxy]phenyl beta-D-glucopyranosiduronic acid

The title compound was prepared by substituting Example 2.55.1 for Example 2.9.1 in Example 2.10. ¹H NMR (300 MHz, dimethyl sulfoxide-d₆) δ ppm 8.22 (t, 1H), 8.05 (s, 1H), 7.99 (d, 1H), 7.76 (d, 1H), 7.61 (d, 1H), 7.46 (t, 1H), 7.35-7.31 (m, 2H), 7.20 (d, 1H), 7.15 (d, 1H), 7.07 (s, 2H), 6.66 (d, 1H), 6.61 (dd, 1H), 5.12 (s, 2H), 5.08 (d, 1H), 4.94 (s, 2H), 4.28 (t, 2H), 4.09 (m, 4H), 4.03 (s, 2H), 3.91 (m, 3H), 3.84 (m, 4H), 3.73 (t, 2H), 3.49 (t, 2H), 3.40 (t, 2H), 3.34 (m, 2H), 3.30 (dd, 2H), 3.26 (m, 2H), 3.06 (q, 2H), 2.13 (s, 3H), 1.39 (bs, 2H), 1.26 (q, 4H), 1.13 (q, 4H), 1.02 (q, 2H), 0.85 (s, 6H). MS (ESI) m/e 1302 (M+H)⁺.

### 2.56 Synthesis of 2-[({[2-(3-[(4-{6-[5-(1,3-benzothiazol-2-ylcarbamoyl)quinolin-3-yl]-2-carboxypyridin-3-yl-5-methyl-1H-pyrazol-1-yl)methyl]-5,7-dimethyltricyclo[3.3.1.13,7]dec-1-yl}oxy)ethyl]carbamoyl}oxy)methyl]-4-[19-(2,5-dioxo-2,5-dioxo-2, hydro-1H-pyrrol-1-yl)-14-oxo-4,7,10-trioxa-13-azanonadec-1-yl]phenyl beta-D-glucopyranosiduronic acid (Synthon QR)

### 2.56.1 3-(1-((3-(2-((((5-(3-(2-(2-(2-aminoethoxy)ethoxy)ethoxy)propyl)-2-(((3R,4S,5S,6S)-6-carboxy-3,45,-trihydroxytetrahydro-2H-pyran-2-yl)oxy)benzyl)oxy)carbonyl)amino)ethoxy)-5,7-dimethyladamantan-1-yl)methyl)-5-methyl-1H-pyrazol-4-yl)-6-(5-(benzo[d]thiazol-2-ylcarbamoyl)quinolin-3-yl)picolinic acid

To a cold (0° C.) solution of (3R,4S,5S,6S)-2-(4-(1-(9H-fluoren-9-yl)-3-oxo-2,7,10,13-tetraoxa-4-azahexadecan-16-yl)-2-((((4-nitrophenoxy)carbonyl)oxy)methyl)phenoxy)-6-(methoxycarbonyl)tetrahydro-2H-pyran-3,4,5-triyl triacetate (56 mg) and Example 1.43.5 (47 mg) in N,N-dimethylformamide (2 mL) was added N,N-diisopropylethylamine (0.026 mL). The reaction was slowly warmed to room temperature and stirred overnight. To the reaction was added water (2 mL) and LiOH H₂O (50 mg), and the mixture was stirred at room temperature for 3 hours. The mixture was acidified with trifluoroacetic acid, filtered and purified by reverse-phase HPLC on a Gilson system (C18 column), eluting with 20-80% acetonitrile in water containing 0.1% trifluoroacetic acid, to provide the title compound.

MS (ESI) m/e 1255.4 (M−H)⁻.

### 2.56.2 2-[({[2-({3-[(4-{6-[5-(1,3-benzothiazol-2-ylcarbamoyl)quinolin-3-yl]-2-carboxypyridin-3-yl}-5-methyl-1H-pyrazol-1-yl)methyl]-5,7-dimethyltricyclo[3.3.1.13,7]dec-1-yl}oxy)ethyl]carbamoyl}oxy)methyl]-4-[19-(2,5-dioxo-2,5-dihydro-1H-pyrrol-1-yl)-14-oxo-4,7,10-trioxa-13-azanonadec-1-yl]phenyl beta-D-glucopyranosiduronic acid

To a solution of Example 2.56.1 (21 mg) in N,N-dimethylformamide (2 mL) was added 2,5-dioxopyrrolidin-1-yl 6-(2,5-dioxo-2,5-dihydro-1H-pyrrol-1-yl)hexanoate (5.24 mg) and N,N-diisopropylethylamine (0.012 mL). The reaction mixture was stirred at room temperature overnight. The mixture was diluted with N,N-dimethylformamide (2 mL), filtered and purified by reverse-phase HPLC on a Gilson system (C18 column), eluting with 20-80% acetonitrile in water containing 0.1% trifluoroacetic acid, to provide the title compound. ¹H NMR (400 MHz, dimethyl sulfoxide-d₆) δ ppm 13.17 (s, 2H), 9.68 (d, 1H), 9.37 (s, 1H), 8.29 (dd, 2H), 8.14 (d, 1H), 8.04 (d, 1H), 8.01-7.88 (m, 2H), 7.82-7.69 (m, 2H), 7.51-7.40 (m, 2H), 7.38-7.29 (m, 1H), 7.17 (t, 1H), 7.13-7.01 (m, 2H), 6.95 (s, 3H), 5.02 (s, 2H), 4.94-4.86 (m, 1H), 3.91-3.79 (m, 4H), 3.33 (td, 9H), 3.29-3.22 (m, 2H), 3.12 (q, 2H), 3.04 (d, 2H), 2.20 (s, 3H), 1.98 (t, 2H), 1.70 (p, 2H), 1.42 (dt, 7H), 1.31-0.89 (m, 13H), 0.82 (s, 7H). MS (ESI) m/e 1448.3 (M−H)⁻.

### 2.57 Synthesis of 4-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.13,7]dec-1-yl}oxy)ethyl](methyl)carbamoyl}oxy)methyl]-3-[4-({N-[6-(2,5-dioxo-2,5-dihydro-1H-pyrrol-1-yl)hexanoyl]-3-sulfo-L-alanyl}amino)butyl]phenyl beta-D-glucopyranosiduronic acid (Synthon SE)

### 2.57.1 (2S,3R,4S,5S,6S)-2-(3-bromo-4-formylphenoxy)-6-(methoxycarbonyl)tetrahydro-2H-pyran-3,4,5-triyl triacetate

A mixture of (3R,4S,5S,6S)-2-bromo-6-(methoxycarbonyl)tetrahydro-2H-pyran-3,4,5-triyl triacetate (2.67 g), 2-bromo-4-hydroxybenzaldehyde (0.90 g) and silver oxide (1.56 g) was stirred in acetonitrile (20 mL) at room temperature protected from light. After 3 hours, the reaction was diluted with dichloromethane (20 mL), filtered through diatomaceous earth, washed with additional dichloromethane (40 mL) and concentrated. The residue was purified by silica gel chromatography, eluting with a gradient of 5% to 50% hexanes/ethyl acetate over 30 minutes, to provide the title compound. MS (ESI) m/e 517.1 (M+H)⁺.

### 2.57.2 (9H-fluoren-9-yl)methyl but-3-yn-1-ylcarbamate

A solution of but-3-yn-1-amine hydrochloride (9 g) and N-ethyl-N-isopropylpropan-2-amine (44.7 mL) was stirred in dichloromethane (70 mL) and the mixture was cooled to 0° C. A solution of (9H-fluoren-9-yl)methyl carbonochloridate (22.06 g) in dichloromethane (35 mL) was added, and the reaction was stirred for 2 hours. The reaction mixture was concentrated. The crude material was deposited onto silica gel, loaded onto a silica gel column and eluted with petroleum diethyl ether/ethyl acetate (10%-25%) to provide the title compound. MS (ESI) m/e 314 (M+Na)⁺.

### 2.57.3 (2S,3R,4S,5S,6S)-2-(3-(4-((((9H-fluoren-9-yl)methoxy)carbonyl)amino)but-1-yn-1-yl)-4-formylphenoxy)-6-(methoxycarbonyl)tetrahydro-2H-pyran-3,4,5-triyl triacetate

Example 2.57.1 (0.389 g), Example 2.57.2 (0.285 g), bis(triphenylphosphine)palladium(II) dichloride (0.053 g), and copper(I) iodide (0.014 g) were weighed into a vial and the vial was flushed with a stream of nitrogen. N,N-diisopropylethylamine (0.263 mL) and N,N-dimethylformamide (1.5 mL) were added, and the reaction was stirred at room temperature overnight. The reaction mixture was diluted with diethyl ether (50 mL) and washed with water (30 mL) and brine (30 mL). The organic layer was dried over magnesium sulfate, filtered, and concentrated. The residue was purified by silica gel chromatography, eluting with a gradient of 5% to 60% ethyl acetate/heptanes over 30 minutes, to provide the title compound. MS (ESI) m/e 728.4 (M+H)⁺.

### 2.57.4 (2S,3R,4S,5S,6S)-2-(3-(4-((((9H-fluoren-9-yl)methoxy)carbonyl)amino)butyl)-4-formylphenoxy)-6-(methoxycarbonyl)tetrahydro-2H-pyran-3,4,5-triyl triacetate

Example 2.57.3 (262 mg) and tetrahydrofuran (10 mL) were added to 10% palladium/C (50 mg) in a 50 mL pressure bottle and the mixture was shaken for 2 hours at room temperature under 30 psi H₂. The reaction mixture was filtered and concentrated to provide the title compound. MS (ESI) m/e 732.5 (M+H)⁺.

### 2.57.5 (2S,3R,4S,5S,6S)-2-(3-(4-((((9H-fluoren-9-yl)methoxy)carbonyl)amino)butyl)-4-(hydroxymethyl)phenoxy)-6-(methoxycarbonyl)tetrahydro-2H-pyran-3,4,5-triyl triacetate

A solution of Example 2.57.4 (0.235 g) in tetrahydrofuran (1.0 mL) and methanol (1.0 mL) was cooled to 0° C., and sodium borohydride (6.07 mg) was added in one portion. The reaction was stirred for 15 minutes and was diluted with ethyl acetate (75 mL) and water (50 mL). The organic layer was separated, washed with brine (50 mL), dried over magnesium sulfate, filtered, and concentrated. The residue was purified by silica gel chromatography, eluting with a gradient of 10% to 70% ethyl acetate/heptanes, to provide the title compound. MS (ESI) m/e 734.5 (M+H)⁺, 2.57.6 (2S,3R,4S,5S,6S)-2-(3-(4-((((9H-fluoren-9-yl)methoxy)carbonyl)amino)butyl)-4-((((4-nitrophenoxy)carbonyl)oxy)methyl)phenoxy)-6-(methoxycarbonyl)tetrahydro-2H-pyran-3,4,5-triyl triacetate

To an ambient solution of Example 2.57.5 (0.148 g) and bis(4-nitrophenyl) carbonate (0.123 g) in N,N-dimethylformamide (1.5 mL) was added N,N-diisopropylethylamine (0.053 mL). After 3 hours, the reaction mixture was concentrated. The residue was purified by silica gel chromatography, eluting with a gradient of 10% to 60% ethyl acetate/hexanes, to provide the title compound. MS (ESI) m/e 899.5 (M+H)⁺.

### 2.57.7 3-(1-((3-(2-((((2-(4-aminobutyl)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic acid

To a solution of Example 1.6.3 (0.101 g) and Example 2.57.6 (0.095 g) in N,N-dimethylformamide (1.0 mL) was added N,N-diisopropvlethylamine (0.055 mL), and the reaction was stirred at room temperature for 3 hours. The reaction was quenched with a mixture of 2,2,2-trifluoroacetic acid (0.204 mL), water (1 mL) and N,N-dimethylformamide (1 mL) and was purified by preparatory reverse-phase HPLC on a Gilson 2020 system using a gradient of 5% to 50% acetonitrile water over 30 minutes. The product-containing fractions were lyophilized to provide the title compound. MS (ESI) m/e 1152.7 (M+H)⁺.

### 2.57.8 3-(l-((3-(2-((((2-(4-((R)-2-amino-3-sulfopropanamido)butyl)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic acid

To a stirred solution of (R)-2-((((9H-fluoren-9-yl)methoxy)carbonyl)amino)-3-sulfopropanoic acid (0.058 g) and O-(7-azabenzotriazol-1-yl)-N,N,N′,N′-tetramethyluronium hexafluorophosphate (0.054 g,) in N,N-dimethylformamide (0.5 mL) was added N,N-diisopropylethylamine (0.051 mL). After stirring for 5 minutes, the mixture was added to a mixture of Example 2.57.7 (0.113 g) and N,N-diisopropylethylamine (0.051 mL) in N,N-dimethylformamide (0.5 mL). After stirring for 2 hours, diethylamine (0.102 mL) was added, and the reaction mixture was stirred for 30 minutes. The reaction mixture was diluted with a solution of 2,2,2-trifluoroacetic acid (0.189 mL) in water (1 mL) and was purified by preparatory reverse-phase HPLC on a Gilson 2020 system using a gradient of 5% to 85% acetonitrile water over 30 minutes. The product-containing fractions were lyophilized to provide the title compound. MS (ESI) m/e 1303.1 (M+H)⁺.

### 2.57.9 4-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)methyl]-3-[4-({N-[6-(2,5-dioxo-2,5-dihydro-1H-pyrrol-1-yl)hexanoyl]-3-sulfo-L-alanyl}amino)butyl]phenyl beta-D-glucopyranosiduronic acid

To a solution of Example 2.57.8 (0.044 g) and 2,5-dioxopyrrolidin-1-yl 6-(2,5-dioxo-2,5-dihydro-1H-pyrrol-1-yl)hexanoate (0.012 g) in N,N-dimethylformamide (0.4 mL) was added N,N-diisopropylethylamine (0.027 mL), and the reaction mixture was stirred for 2 hours at room temperature. The reaction mixture was quenched with a mixture of 2,2,2-trifluoroacetic acid (0.060 mL), water (1 mL) and N,N-dimethylformamide (1 mL) and purified by preparatory reverse-phase HPLC on a Gilson 2020 system using a gradient of 5% to 50% acetonitrile water over 30 minutes. The product-containing fractions were lyophilized to provide the title compound. ¹H NMR (400 MHz, dimethyl sulfoxide-d₆) δ ppm 13.10 (s, 1H), 9.02 (s, 1H), 8.38 (dd, 1H), 8.27-8.14 (m, 3H), 8.07 (d, 1H), 8.02 (d, 1H), 7.94 (d, 1H), 7.82 (dd, 2H), 7.79-7.66 (m, 2H), 7.53-7.44 (m, 1f), 7.48 (s, 1H), 7.37 (t, 1H), 7.23 (d, 1H), 6.98 (s, 2H), 6.88 (d, 1H), 6.82 (dd, 1H), 5.04 (d, 1H), 5.00 (s, 2H), 4.29 (q, 2H), 3.57 (s, 2H), 3.44 (s, 4H), 3.41 (d, 1H), 3.40-3.27 (m, 3H), 3.30-3.21 (m, 2H), 3.03 (t, 2H), 2.85 (s, 3H), 2.79 (dd, 1H), 2.70 (dd, 1H), 2.58 (s, 2H), 2.23 (s, 3H), 2.06 (t, 2H), 1.53-1.41 (m, 5H), 1.42 (s, 6H), 1.26 (s, 2H), 1.25-1.07 (m, 8H), 0.85 (s, 6H). MS (ESI) m/e 1494.1 (M−H)⁻.

### 2.58 Synthesis of 2-6-[2-({3-[(4-6-[8-(1,3-benzothiazol-2-ylcarbamoyl)-3,4-dihydroisoquinolin-2(1H)-yl]-2-carboxypyridin-3-yl)-5-methyl-1H-pyrazol-1-yl)methyl]-5,7-dimethyltricyclo[3.3.1.13,7]dec-1-yl)oxy)ethyl]-2-methyl-3,3-dioxido-7-oxo-8-oxa-3lambda6-thia-2,6-diazanonan-9-yl}-5-(4-([(2,5dioxo-2,5-dihydro-1H-pyrrol-1-yl)acetyl]amino)butyl)phenyl beta-D-glucopyranosiduronic acid (Synthon UH)

### 2.58.1 (9H-fluoren-9-yl)methyl but-3-yn-1-ylcarbamate

A solution of but-3-yn-1-amine hydrochloride (9 g) and N,N-diisopropylethylamine (44.7 mL) was stirred in dichloromethane (70 mL) and the mixture was cooled to 0° C. A solution of (9H-fluoren-9-yl)methyl carbonochloridate (22.06 g) in dichloromethane (35 mL) was added, and the reaction mixture was stirred for 2 hours. The reaction mixture was concentrated, and the residue was purified by silica gel chromatography, eluting with petroleum ether in ethyl acetate (10%-25%) to provide the title compound. MS (ESI) m/e 314 (M+Na)⁺.

### 2.58.2 (2S,3S,4S,5R,6S)-methyl 6-(5-(4-(((9H-fluoren-9-yl)methoxy)carbonylamino)but-1-ynyl)-2-formylphenoxy)-3,4,5-triacetoxy-tetrahydro-2H-pyran-2-carboxylate

Example 2.58.3 (2.7 g), Example 2.58.1 (2.091 g), bis(triphenylphosphine)palladium(II) chloride (0.336 g) and copper(I) iodide (0.091 g) were weighed into a vial and flushed with a stream of nitrogen. Triethylamine (2.001 mL) and tetrahydrofuran (45 mL) were added, and the reaction was stirred at room temperature. After stirring for 16 hours, the reaction mixture was diluted with ethyl acetate (200 mL) and washed with water (100 mL) and brine (100 mL). The organic layer was dried over magnesium sulfate, filtered, and concentrated. The residue was purified by silica gel chromatography, eluting with petroleum ether in ethyl acetate (10%-50%), to provide the title compound. MS (ESI) m/e 750 (M+Na)⁺.

### 2.583 (2S,3S,4S,5R,6S)-methyl 6-(5-(4-(((9H-fluoren-9-yl)methoxy)carbonylamino)butyl)-2-formylphenoxy)-3,4,5-triacetoxy-tetrahydro-2H-pyran-2-carboxylate

Example 2.58.2 (1.5 g) and tetrahydrofuran (45 mL) were added to 10% Pd—C(0.483 g) in a 100 mL pressure bottle and the mixture was stirred for 16 hours under 1 atm H₂ at room temperature. The reaction mixture was filtered and concentrated to provide the title compound. MS (ESI) m/e 754 (M+Na)⁺.

### 2.58.4 (2S,3S,4S,5R,6S)-methyl 6-(5-(4-(((9H-fluoren-9-yl)methoxy)carbonylamino)butyl)-2-(hydroxymethyl)phenoxy)-3,4,5-triacetoxy-tetrahydro-2H-pyran-2-carboxylate

A solution of Example 2.58.3 (2.0 g) in tetrahydrofuran (7.00 mL) and methanol (7 mL) was cooled to 0° C., and NaBH₄ (0.052 g) was added in one portion. After 30 minutes, the reaction mixture was diluted with ethyl acetate (150 mL) and water (100 mL). The organic layer was separated, washed with brine (100 mL), dried over magnesium suflate, filtered, and concentrated. The residue was purified by silica gel chromatography, eluting with petroleum ether in ethyl acetate (10%-40%), to provide the title compound. MS (ESI) m/e 756 (M+Na)⁺.

### 2.58.5 (2S,3S,4S,5R,6S)-methyl 6-(5-(4-(((9H-fluoren-9-yl)methoxy)carbonylamino)butyl)-2-(((4-nitrophenoxy)carbonyloxy)methyl)phenoxy)-3,4,5-triacetoxy-tetrahydro-2H-pyran-2-carboxylate

To a solution of Example 2.58.4 (3.0 g) and bis(4-nitrophenyl) carbonate (2.488 g) in dry acetonitrile (70 mL) at 0° C., was added N,N-diisopropylethylamine (1.07 mL). After stirring at room temperature for 16 hours, the reaction mixture was concentrated to give a residue, which was purified by silica gel chromatography, eluting with petroleum ether in ethyl acetate (10%-50%), to provide the title compound. MS (ESI) m/e 921 (M+Na)⁺.

### 2.58.6 3-(1-((3-(2-((((4-(4-aminobutyl)-2-(((2R,3S,4R,5R,6R)-6-carboxy-3,4,5-trihydroxytetrahydro-2H-pyran-2-yl)oxy)benzyl)oxy)carbonyl)(2-(N,N-dimethylsulfamoyl)ethyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid

To a cold (0° C.) solution of Example 2.58.5 (40.8 mg) and Example 1.36 (40 mg) in N,N-dimethylformamide (4 mL) was added N,N-diisopropvlethylamine (0.026 mL). The reaction mixture was slowly warmed to room temperature and stirred overnight. To the reaction mixture was added water (2 mL) and LiOH H₂O (50 mg), and the mixture was stirred at room temperature for 3 hours. The mixture was acidified with trifluoroacetic acid, filtered and purified by reverse-phase HPLC on a Gilson system (C18 column), eluting with 20-80% acetonitrile in water containing 0.1% trifluoroacetic acid, to provide the title compound.

MS (ESI) m/e 1278.7 (M−H)⁻,

### 2.58.7 2-(6-[2-({3-[(4-6-[8-(1,3-benzothiazol-2-ylcarbamoyl)-3,4-dihydroisoquinolin-2(1H)-yl]-2-carboxypyridin-3-yl)-5-methyl-1H-pyrazol-1-yl)methyl]-5,7-dimethyltricyclo[3.3.1.13,7]dec-1-yl}oxy)ethyl]-2-methyl-3,3-dioxido-7-oxo-8-oxa-3lambda6-thia-2,6-diazanonan-9-yl)-5-(4-{[(2,5-dioxo-2,5-dihydro-1H-pyrrol-1-yl)acetyl]amino}butyl)phenyl beta-D-glucopyranosiduronic acid

To a solution of Example 2.58.6 (35.1 mg) in N,N-dimethylformamide (4 mL) was added 2,5-dioxopyrrolidin-1-yl 2-(2,5-dioxo-2,5-dihydro-1H-pyrrol-1-yl)acetate (6.93 mg) and N,N-diisopropylethylamine (0.026 mL). The reaction mixture was stirred at room temperature overnight. The mixture was diluted with N,N-dimethylformamide (2 mL), filtered and purified by reverse-phase HPLC on a Gilson system (C18 column), eluting with 20-80% acetonitrile in water containing 0.1% trifluoroacetic acid, to provide the title compound. ¹H NMR (400 MHz, dimethyl sulfoxide-d₆) δ ppm 12.85 (s, 1H), 8.02 (dd, 2H), 7.76 (d, 1H), 7.58 (d, 1H), 7.53-7.37 (m, 3H), 7.32 (td, 2H), 7.24 (s, 1H), 7.16 (dd, 1H), 7.04 (s, 2H), 6.99-6.87 (m, 2H), 6.81 (d, 1H), 5.08 (d, 2H), 4.99 (d, 1H), 4.92 (s, 2H), 3.95 (s, 2H), 3.86 (q, 3H), 3.47-3.14 (m, 9H), 2.99 (dt, 4H), 2.72 (s, 3H), 2.60 (s, 3H), 2.06 (s, 3H), 1.49 (p, 2H), 1.41-1.27 (m, 4H), 1.29-0.86 (m, 10H), 0.80 (d, 7H). MS (ESI) m/e 1413.4 (M−H)⁻.

### 2.59 Synthesis of 6-[8-(1,3-benzothiazol-2-ylcarbamoyl)-3,4-dihydroisoquinolin-2(1H)-yl]-3-(1-{[3-(2-{({[12-{[(2S,3R,4S,5S,6S)-6-carboxy-3,4,5-trihydroxytetrahydro-2H-pyran-2-yl]oxy}-4-(4-{[(2,5-dioxo-2,5-dihydro-1H-pyrrol-1-yl)acetyl]amino}butyl)benzyl]oxy}carbonyl) [3-(dimethylamino)-3-oxopropyl]amino}ethoxy)-5,7-dimethyltricyclo[3.3.1.13,7]dec-1-yl]methyl}-5-methyl-1H-pyrazol-4-yl)pyridine-2-carboxylic acid (Synthon UI)

### 2.59.1 3-(1-((3-(2-((((4-(4-aminobutyl)-2-(((2R,3S,4R,5R,6R)-6-carboxy-3,4,5-trihydroxytetrahydro-2H-pyran-2-yl)oxy)benzyl)oxy)carbonyl)(3-(dimethylamino)-3-oxopropyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid

The title compound was prepared as described in Example 2.58.6, substituting Example 1.36 with Example 1.38. MS (ESI) m/e 1243.7 (M+H)⁺.

### 2.59.2 6-[8-(1,3-benzothiazol-2-ylcarbamoyl)-3,4-dihydroisoquinolin-2(1H)-yl]-3-(1-({[3-(2-{[[2-([(2S,3R,4S,5S,6S)-6-carboxy-3,4,5-trihydroxytetrahydro-2H-pyran-2-yl]oxy}-4-(4-{[(2,5-dioxo-2,5-dihydro-1H-pyrrol-1-yl)acetyl]amino}butyl)benzyl]oxy}carbonyl)[3-(dimethylamino)-3-oxopropyl] amino}ethoxy)-5,7-dimethyltricyclo[0.3.1.13,7]dec-1-yl]methyl}-5-methyl-1H-pyrazol-4-yl)pyridine-2-carboxylic acid

The title compound was prepared as described in Example 2.58.7, substituting Example 2.58.6 with Example 2.59.1. ¹H NMR (400 MHz, dimethyl sulfoxide-d₆) δ ppm 8.02 (dd, 2H), 7.76 (d, 1H), 7.58 (d, 1H), 7.44 (ddd, 3H), 7.32 (td, 2H), 7.24 (s, 1H), 7.13 (dd, 1H), 7.04 (s, 2H), 6.99-6.86 (m, 2H), 6.81 (d, 1H), 5.06 (d, 2H), 4.98 (d, 1H), 4.92 (s, 2H), 3.95 (s, 2H), 3.85 (q, 3H), 3.77 (d, 2H), 3.39 (q, 5H), 3.27 (q, 4H), 2.99 (dt, 4H), 2.88 (s, 2H), 2.81-2.66 (m, 5H), 2.06 (d, 3H), 1.50 (p, 2H), 1.34 (dd, 4H), 1.27-0.85 (m, 9H), 0.79 (d, 6H). MS (ESI) m/e 1401.3 (M+H)⁺.

### 2.60 Synthesis of 2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.13,7]dec-1-yl}oxy)ethyl](2-sulfamoylethyl)carbamoyl}oxy)methyl]-5-(4-({[2,5-dioxo-2,5-dihydro-1H-pyrrol-1-yl)acetyl]amino}butyl)phenyl beta-D-glucopyranosiduronic acid (Synthon US)

### 2.60.1 3-(1-((3-(2-((((4-(4-aminobutyl)-2-(((2R,3S,4R,5R,6R)-6-carboxy-3,4,5-trihydroxytetrahydro-2H-pyran-2-yl)oxy)benzyl)oxy)carbonyl)(2-sulfamoylethyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid

The title compound was prepared as described in Example 2.58.6, substituting Example 1.36 with Example 1.18.20. MS (ESI) m/e 1251.2 (M+H)⁺.

### 2.60.2 2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.13,7]dec-1-yl}oxy)ethyl](2-sulfamoylethyl)carbamoyl}oxy)methyl]-5-(4-{[(2,5-dioxo-2,5-dihydro-1H-pyrrol-1-yl)acetyl]amino}butyl)phenyl beta-D-glucopyranosiduronic acid

The title compound was prepared as described in Example 2.58.7, substituting Example 2.58.6 with Example 2.60.1. ¹H NMR (400 MHz, dimethyl sulfoxide-d₆) δ ppm 12.84 (s, 2H), 8.04 (dd, 2H), 7.77 (d, 1H), 7.60 (d, 1H), 7.53-7.38 (m, 3H), 7.38-7.30 (m, 2H), 7.26 (s, 1H), 7.16 (d, 1H), 7.05 (s, 2H), 6.96-6.77 (m, 5H), 5.09 (s, 2H), 5.00 (d, 1H), 4.94 (s, 2H), 3.97 (s, 2H), 3.87 (q, 3H), 3.48-3.16 (m, 5H), 3.09-2.94 (m, 4H), 2.07 (s, 3H), 1.50 (d, 2H), 1.36 (d, 3H), 1.29-0.88 (m, 9H), 0.81 (d, 7H). MS (ESI) m/e 1385.5 (M−H)⁻.

### 2.61 Synthesis of 6-[8-(1,3-benzothiazol-2-ylcarbamoyl)-3,4-dihydroisoquinoin-2(1H)-yl-3-(1-{13-(2-{({[2-{[(2S,3R,4S,5S,6S)-6-carboxy-3,4,5-trihydroxytetrahydro-2H-pyran-2-yl]oxy}-4-(4-{[(2,5-dioxo-2,5-dihydro-1H-pyrrol-1-yl)acetyl]amino}butyl)benzyl]oxy}carbonyl)[3-(methylamino)-3-oxopropyl]amino}ethoxy)-5,7-dimethyltricyclo[3.3.1.13,7]dec-1-yl]methyl}-5-methyl-1H-pyrazol-4-yl)pyridine-2-carboxylic acid (Synthon UY)

### 2.61.1 3-(1-((3-(2-((((4-(4-aminobutyl)-2-(((2R,3S,4R,5R,6R)-6-carboxy-3,4,5-trihydroxytetrahydro-2H-pyran-2-yl)oxy)benzyl)oxy)carbonyl)(3-(methylamino)-3-oxopropyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid

The title compound was prepared as described in Example 2.58.6, substituting Example 1.36 with Example 1.39. MS (EST) m/e 1228.8 (M+H)⁺,

### 2.61.2 6-[8-(1,3-benzothiazol-2-ylcarbamoyl)-3,4-dihydroisoquinolin-2(1H)-yl]-3-(1-{[3-(2-{([2-{[(2S,3R,4S,5S,6S)-6-carboxy-3,4,5-trihydroxytetrahydro-2H-pyran-2-yl]oxy}-4-(4-{[(2,5-dioxo-2,5-dihydro-1H-pyrrol-1-yl)acetyl]amino}butyl)benzyl]oxy}carbonyl)[3-(methylamino)-3-oxopropyl]amino}ethoxy)-5,7-dimethyltricyclo[3.3.1.13,7]dec-1-yl]methyl)-5-methyl-1H-pyrazol-4-yl)pyridine-2-carboxylic acid

The title compound was prepared as described in Example 2.58.7, substituting Example 2.58.6 with Example 2.61.1. ¹H NMR (501 MHz, dimethyl sulfoxide-d₆) δ ppm 12.83 (s, 1H), 8.06 (s, 1H), 8.01 (dd, 1H), 7.77 (d, 1H), 7.71 (d, OH), 7.60 (d, 1H1), 7.45 (tdd, 3H), 7.38-7.29 (m, 2H), 7.26 (s, 1H), 7.15 (d, 1H), 7.05 (d, 1H), 6.96-6.90) (m, 2H), 6.82 (d, 1H), 5.07 (s, 2H), 5.01 (t, 1H), 4.94 (s, 2H), 3.97 (s, 2H), 3.87 (q, 3H), 3.79 (d, 2H), 3.28 (p, 2H1), 3.09-2.93 (m, 3H1), 2.52 (d, 3H1), 2.35-2.26 (m, 2H), 2.07 (d, 2H), 1.60-1.44 (m, 2H), 1.34 (d, 3H), 1.29-0.88 (m, 6H), 0.81 (d, 5H). MS (ESI) in/c 1363.5 (M−H)⁻.

### 2.62 Synthesis of 3-{1-[(3-(2-[(3-amino-3-oxopropyl)({[12{[(2S,3R,4S,5S,6S)-6-carboxy-3,4,5-trihydroxytetrahydro-2H-pyran-2-yl]oxy}-4-(4-{[2,5-dioxo-2,5-dihydro-1H-pyrrol-1-yl)acetyl]amino}butyl)benzyl]oxy}carbonyl)amino]ethoxy)-5,7-dimethyltricyclo[3.3.1.1 3,7]dec-1-yl)methyl]-5-methyl-1H-pyrazol-4-yl}-6-[8-(1,3-benzothiazol-2-ylcarbamoyl)-3,4-dihydroisoquinolin-2(1H)-yl]pyridine-2-carboxylic acid (Synthon UX)

### 2.62.1 3-(1-((3-(2-((3-amino-3-oxopropyl)(((4-(4-aminobutyl)-2-(((2R,3S,4R,5R,6R)-6-carboxy-3,45,-trihydroxytetrahydro-2H-pyran-2-yl)oxy)benzyl)oxy)carbonyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid

The title compound was prepared as described in Example 2.58.6, substituting Example 1.36 with Example 1.32.2. MS (ESI) m/e 1214.6 (M+H)⁺.

### 2.62.2 3-{1-[(3-{2-[(3-amino-3-oxopropyl)({[2-{[(2S,3R,4S,5S,6S)-6-carboxy-3,4,5-trihydroxytetrahydro-2H-pyran-2-yl]oxy}-4-(4-{[(2,5-dioxo-2,5-dihydro-1H-pyrrol-1-yl)acetyl]amino}butyl)benzyl]oxy}carbonyl)amino]ethoxy}-5,7-dimethyltricyclo[3.3.1.13,7]dec-1-yl)methyl]-5-methyl-1H-pyrazol-4-yl}-6-[8-(1,3-benzothiazol-2-ylcarbamoyl)-3,4-dihydroisoquinolin-2(1H)-yl]pyridine-2-carboxylic acid

The title compound was prepared as described in Example 2.58.7, substituting Example 2.58.6 with Example 2.62.1. ¹H NMR (501 MHz, dimethyl sulfoxide-d₆) δ ppm 12.83 (s, 2H), 8.06 (s, 1H), 8.01 (d, 1H), 7.77 (d, 1H), 7.60 (d, 1H), 7.53-7.38 (m, 3H), 7.34 (q, 2H), 7.26 (s, 1H), 7.15 (d, 1H), 7.05 (s, 2H), 6.93 (d, 2H), 6.87-6.73 (m, 2H), 5.07 (d, 2H), 5.04-4.97 (m, 1H1), 4.94 (s, 2H), 3.97 (s, 2H), 3.87 (q, 3H), 3.79 (d, 2H), 3.29 (t, 3H), 3.10-2.95 (m, 4H), 2.32 (p, 2H), 2.07 (d, 3H), 1.51 (dd, 2H), 1.36 (dd, 5H), 1.30-0.86 (m, 8H), 0.81 (d, 6H). MS (ESI) m/e 1349.5 (M−H)⁻.

### 2.63 Synthesis of 2-[({[2-({3-[(4-{6-[3-(1,3-benzothiazol-2-ylcarbamoyl)-1H-indol-5-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)methyl]-5-(4-{[(2,5-dioxo-2,5-dihydro-1H-pyrrol-1-yl)acetyl]amino}butyl)phenyl beta-D-glucopyranosiduronic acid (Synthon WZ)

### 2.63.1 3-(1-((3-(2-((((4-(4-aminobutyl)-2-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(3-(benzo[d]thiazol-2-ylcarbamoyl)-1H-indol-5-yl)picolinic acid

The title compound was prepared by substituting Example 1.34.5 for Example 1.12.10 and Example 2.58.5 for Example 2.11.6 in Example 2.11.7.

### 2.63.2 2-[({[2-({3-[(4-{6-[3-(1,3-benzothiazol-2-ylcarbamoyl)-1H-indol-5-yl]-2-carboxypyridin-3-yl}-5-methyl-1H-pyrazol-1-yl)methyl]-5,7-dimethyltricyclo[3.3.1.13,7]dec-1-yl}oxy)ethyl](methyl)carbamoyl}oxy)methyl]-5-(4-{[(2,5-dioxo-2,5-dihydro-1H-pyrrol-1-yl))acetyl]amino}butyl)phenyl beta-D-glucopyranosiduronic acid

The title compound was prepared by substituting Example 2.63.1 for Example 2.9.1 in Example 2.10. ¹H NMR (400 MHz, dimethyl sulfoxide-d₆) δ ppm 12.47 (bs, 1H), 12.16 (d, 1H), 9.01 (s, 1H), 8.69 (d, 1H), 8.11-8.04 (m, 4H), 7.99 (d, 1H), 7.76 (d, 1H), 7.64 (d, 1H), 7.48 (s, 1H), 7.45 (t, 1H), 7.31 (t, 1H), 7.19 (t, 1H), 7.07 (s, 1H), 6.94 (s, 1H), 6.86 (d, 1H), 5.10 (s, 2H), 5.03 (d, 1H), 3.99 (s, 2H), 3.90 (m, 3H), 3.48 (m, 3H), 3.28 (m, 2H), 3.05 (m, 4H), 2.93 (s, 2H), 2.88 (s, 2H), 2.54-2.53 (m, 2H), 2.24 (s, 3H), 1.54 (m, 2H), 1.40 (m, 4H), 1.30-1.22 (m, 6H), 1.20-1.14 (m, 6H), 1.11-0.96 (m, 2H), 0.87 (d, 6H). MS (ESI) m/e 1300 (M+Na), 1276 (M−H)⁻.

### 2.64 Synthesis of 2-[({[2-({3-[(4-{6-[1-(1,3-benzothiazol-2-ylcarbamoyl)-5,6-dihydroimidazo[1,5-a]pyrazin-7(8H)-yl]-2-carboxypyridin-3-yl}-5-methyl-1H-pyrazol-1-yl)methyl]-5,7-dimethyltricyclo[3.3.1.13,7]dec-1-yl}oxy)ethyl]carbamoyl}oxy)methyl]-5-(4-{[(2,5-dioxo-2,5-dihydro-1H-pyrrol-1-yl)acetyl]amino}butyl)phenyl beta-D-glucopyranosiduronic acid (Synthon XO)

### 2.64.1 3-(1-((3-(2-((((4-(4-aminobutyl)-2-(((2S,3R,4S,5S,6S)-6-carboxy-3,4,5-trihydroxytetrahydro-2H-pyran-2-yl)oxy)benzyl)oxy)carbonyl)amino)ethoxy)-5,7-dimethyladamantan-1-yl)methyl)-5-methyl-1H-pyrazol-4-yl)-6-(1-(benzo[d]thiazol-2-ylcarbamoyl)-5,6-dihydroimidazo[1,5-a]pyrazin-7(8H)-yl)picolinic acid

The title compound was prepared by substituting Example 1.4.10 for Example 1.12.10 and Example 2.58.5 for Example 2.11.6 in Example 2.11.7. MS (ESI) m/e 1133 (M+H)⁺, 1131 (M−H)⁻.

### 2.64.2 2-[({[2-({3-[4-{6-[1-(1,3-benothiazol-2-ylcarbamoyl)-5,6-dihydroimidazo[1,5-a]pyrazin-7(8H)-yl]-2-carboxypyridin-3-yl}-5-methyl-1H-pyrazol-1-yl)methyl]-5,7-dimethyltricyclo[3.3.1.13,7]dec-1-yl}oxy)ethyl]carbamoyl}oxy)methyl]-5-(4-([(2,5-dioxo-2,5-dihydro-1H-pyrrol-1-yl)acetyl]amino)butyl)phenyl beta-D-glucopyranosiduronic acid

The title compound was prepared by substituting Example 2.64.1 for Example 2.9.1 in Example 2.10. ¹H NMR (400 MHz, dimethyl sulfoxide-d₆) δ ppm 8.08 (t, 1H), 8.01 (s, 1H), 7.99 (d, 1H), 7.76 (d, 1H), 7.61 (d, 1H), 7.46 (t, 1H), 7.34 (s, 1H), 7.33 (t, 1H), 7.17 (m, 3H), 7.08 (s, 2H), 6.92 (s, 1H), 6.84 (d, 1H), 5.12 (s, 2H), 5.05 (s, 2H), 5.02 (d, 1H), 4.27 (m, 2H), 4.10 (m, 2H), 3.99 (s, 2H), 3.91 (m, 2H), 3.84 (s, 2H), 3.70 (m, 2H), 3.42 (t, 2H), 3.35 (t, 2H), 3.30 (t, 2H), 3.06 (m, 5H), 2.53 (m, 2H), 2.14 (s, 3H), 1.53 (m, 2H), 1.43-1.35 (m, 4H), 1.27 (m, 4H), 1.14 (q, 4H), 1.03 (dd, 2H), 0.86 (s, 6H). MS (ESI) m/e 1270 (M+H)⁺, 1268 (M−H)⁻,

### 2.65 Synthesis of (6S)-2,6-anhydro-6-(2-{2-[({[12-({3-[(4-6-[1-(1,3-benzothiazol-2-ylcarbamoyl)-5,6-dihydroimidazo[1,5-a]pyrazin-7(8H)-yl]-2-carboxypyridin-3-yl})-5-methyl-1H-pyrazol-1-yl)methyl]-5,7-dimethyltricyclo[3.3.1.13,7]dec-1-yl}oxy)ethyl]carbamoyl}oxy)methyl]-5-((N-[(2,5-dioxo-2,5-dihydro-1H-pyrrol-1-yl)acetyl]-L-valyl-L-alanyl)amino)phenyl)ethyl)-L-gulonic acid (Synthon XW)

### 2.65.1 (3R,4S,5R,6R)-3,4,5-tris(benzyloxy)-6-(benzyloxymethyl)-tetrahydropyran-2-one

To a solution of (3R,4S,5R,6R)-3,4,5-tris(benzyloxy)-6-((benzyloxy)methyl)tetrahydro-2H-pyran-2-ol (75 g) in dimethyl sulfoxide (400 mL) at 0° C., was added acetic anhydride (225 mL). The mixture was stirred for 16 hours at room temperature before cooled to 0° C. A large volume of water was added, and the stirring was stopped and the reaction mixture was allowed to settle for 3 hours (the crude lactone was at the bottom of the flask). The supernatant was removed, and the crude mixture was diluted with ethyl acetate, washed 3 times with water, neutralized with saturated aqueous solution of NaHCO₃, and washed again twice with water. The organic layer was then dried over magnesium sulfate, filtered and concentrated to provide the title compound. MS (ESI) m % e 561 (M+Na)⁺.

### 2.65.2 (3R,4S,5R,6R)-3,4,5-tris(benzyloxy)-6-(benzyloxymethyl)-2-ethynyl-tetrahydro-2H-pyran-2-ol

To a solution of ethynyltrimethylsilane (18.23 g) in tetrahydrofuran (400 mL) under nitrogen and chilled in a dry ice/acetone bath (internal temp −65° C.) was added 2.5M BuLi in hexane (55.7 mL) dropwise, keeping the temperature below −60° C. The mixture was stirred in a cold bath for 40 minutes, followed by an ice-water bath (internal temp rose to 0.4° C.) for 40 minutes, and finally cooled to −75° C., again. A solution of Example 2.55.1 (50 g) in tetrahydrofuran (50 mL) was added dropwise, keeping the internal temperature below −70° C. The mixture was stirred in a dry ice/acetone bath for an additional 3 hours. The reaction mixture was quenched with saturated aqueous NaHCO₃ solution (250 mL). The mixture was allowed to warm to room temperature, extracted with ethyl acetate (3×300 mL), dried over MgSO₄, filtered, and concentrated in vacuo to provide the title compound. MS (ESI) m/e 659 (M+Na)⁺.

### 2.65.3 trimethyl(((3S,4R,5R,6R)-3,4,5-tris(benzyloxy)-6-(benzyloxymethyl)-tetrahydro-2H-pyran-2-yl)ethynyl)silane

To a mixture of Example 2.65.2 (60 g) in acetonitrile (450 mL) and dichloromethane (150 mL) at −15° C. in an ice-salt bath was added triethylsilane (81 mL) dropwise, followed by addition of boron trifluoride diethyl ether complex (40.6 mL) at such a rate that the internal temperature did not exceed −10° C. The mixture was stirred between −15° C. and −10 (C for 2 hours. The reaction mixture was quenched with saturated aqueous NaHCO₃ solution (275 mL) and stirred for 1 hour at room temperature. The mixture was extracted with ethyl acetate (3×550 mL). The combined extracts were dried over MgSO₄, filtered, and concentrated. The residue was purified by flash chromatography eluting with a gradient of 0% to 7% ethyl acetate/petroleum ether to provide the title compound. MS (ESI) m/e 643 (M+Na).

### 2.65.4 (2R,3R,4R,5S)-3,4,5-tris(benzyloxy)-2-(benzyloxymethyl)-6-ethynyl-tetrahydro-2H-pyran

To a mixed solution of Example 2.65.3 (80 g) in dichloromethane (200 mL) and methanol (1000 mL) was added 1N aqueous NaOH solution (258 mL). The mixture was stirred at room temperature for 2 hours. The solvent was removed. The residue was then partitioned between water and dichloromethane. The extracts were washed with brine, dried over Na₂SO₄, filtered, and concentrated to provide the title compound. MS (ESI) m/e 571 (M+Na)⁺.

### 2.65.5 (2R,3R,4R,5S)-2-(acetoxymethyl)-6-ethynyl-tetrahydro-2H-pyran-3,4,5-triyl triacetate

To a solution of Example 2.65.4 (66 g) in acetic anhydride (500 mL) cooled by an ice/water bath was added boron trifluoride diethyl ether complex (152 mL) dropwise. The mixture was stirred at room temperature for 16 hours, cooled with an ice/water bath and neutralized with saturated aqueous NaHCO₃ solution. The mixture was extracted with ethyl acetate (3×500 mL), dried over Na₂SO₄ and concentrated in vacuo. The residue was purified by flash chromatography eluting with a gradient of 0% to 30% ethyl acetate/petroleum ether to provide the title compound. MS (ESI) m/e 357 (M+H)⁺.

### 2.65.6 (3R,4R,5S,6R)-2-ethynyl-6-(hydroxymethyl)-tetrahydro-2H-pyran-3,4,5-triol

To a solution of Example 2.65.5 (25 g) in methanol (440 mL) was added sodium methanolate (2.1 g). The mixture was stirred at room temperature for 2 hours, then neutralized with 4M HCl in dioxane. The solvent was removed, and the residue was adsorbed onto silica gel and loaded onto a silica gel column. The column was eluted with a gradient of 0 to 100% ethyl acetate/petroleum ether then 0% to 12% methanol/ethyl acetate to provide the title compound. MS (ESI) m/e 211 (M+Na)*.

### 2.65.7 (2S,3S,4R,5R)-6-ethynyl-3,4,5-trihydroxy-tetrahydro-2H-pyran-2-carboxylic acid

A three-necked round bottom flask was charged with Example 2.65.6 (6.00 g), KBr (0.30 g), tetrabutylamnnonium bromide (0.41 g) and 60 mL of saturated aqueous NaHCO₃ solution. (2.2,6,6-Tetramethylpiperidin-1-yl)oxidanyl (0.15 g) in 60 mL dichloromethane was added. The mixture was stirred vigorously and cooled in an ice-salt bath to −2° C., internal temperature. A solution of brine (12 mL), aqueous NaHCO₃ solution (24 mL) and NaOCl (154 mL) was added dropwise such that the internal temperature was maintained below 2° C. The pH of the reaction mixture was maintained in the 8.2-8.4 range with the addition of solid Na₂CO₃. After a total of 6 hours the reaction was cooled to 3° C., internal temperature and ethanol (˜20 mL) was added dropwise and was stirred for ˜30 minutes. The mixture was transferred to a separatory funnel, and the dichloromethane layer was discarded. The pH of the aqueous layer was adjusted to 2-3 using 1 M aqueous HCl. The aqueous layer was then concentrated to dryness. Methanol (100 mL was) added to the dry solid, and the slurry was stirred for ˜30 minutes. The mixture was filtered over a pad of diatomaceous earth, and the residue in the funnel was washed with ˜100 mL of methanol. The filtrate was concentrated under reduced pressure to obtain the title compound.

### 2.65.8 (2S,3S,4R,5R)-methyl 6-ethynyl-3,4,5-trihydroxytetrahydro-2H-pyran-2-carboxylate

A 500 mL three-necked round bottom flask was charged with a suspension of Example 2.65.7 (6.45 g) in methanol (96 mL) and was cooled in an ice-salt-bath with internal temperature of −1° C. Neat thionyl chloride (2.79 mL) was carefully added. The internal temperature kept rising throughout the addition but did not exceed 10° C. The reaction was allowed to slowly warm up to 15-20° C. over 2.5 hours. After 2.5 hours, the reaction was concentrated to provide the title compound.

### 2.65.9 (3S,4R,5S,6S)-2-ethynyl-6-(methoxycarbonyl)tetrahydro-2H-pyran-3,4,5-triyl triacetate

Example 2.65.8 (6.9 g) as a solution in N,N-dimethylformamide (75 mL) was added 4-dimethylaminopyridine (0.17 g) and acetic anhydride (36.1 mL). The suspension was cooled in an ice-bath and pyridine (18.04 mL) was added via syringe over 15 minutes. The reaction was allowed to warm to room temperature overnight. Additional acetic anhydride (12 mL) and pyridine (6 mL) were added and stirring was continued for an additional 6 hours. The reaction was cooled in an ice-bath and 250 mL of saturated aqueous NaHCO₃ solution was added and stirred for 1 hour. Water (100 mL) was added, and the mixture was extracted with ethyl acetate. The organic extract was washed twice with saturated CuSO₄ solution, dried and concentrated. The residue was purified by flash chromatography, eluting with 50% ethyl acetate/petroleum ether to provide the title compound. ¹H NMR (500 MHz, methanol-d₄) δ ppm 5.29 (t, 1H), 5.08 (td, 2H), 4.48 (dd, 1H), 4.23 (d, 1H), 3.71 (s, 3H), 3.04 (d, 1H), 2.03 (s, 3H), 1.99 (s, 3H), 1.98 (s, 4H). MS (ESI) m/e 359.9 (M+NH₄)⁺.

### 2.65.10 2-iodo-4-nitrobenzoic acid

A 3 L fully jacketed flask equipped with a mechanical stirrer, temperature probe and an addition funnel under a nitrogen atmosphere, was charged with 2-amino-4-nitrobenzoic acid (69.1 g, Combi-Blocks) and sulfuric acid, 1.5 M aqueous (696 mL). The resulting suspension was cooled to 0° C., internal temperature, and a solution of sodium nitrite (28.8 g) in water (250 mL) was added dropwise over 43 minutes with the temperature kept below 1° C. The reaction mixture was stirred at ca. 0° C. for 1 hour. A solution of potassium iodide (107 g) in water (250 mL) was added dropwise over 44 minutes with the internal temperature kept below 1° C. (Initially addition was exothermic and there was gas evolution). The reaction mixture was stirred 1 hour at 0° C. The temperature was raised to 20° C., and then stirred at ambient temperature overnight. The reaction mixture became a suspension. The reaction mixture was filtered, and the collected solid was washed with water. The wet solid (˜108 g) was stirred in 10% sodium sulfite (350 mL, with ˜200 mL water used to wash in the solid) for 30 minutes. The suspension was acidified with concentrated hydrochloric acid (35 mL), and the solid was collected by filtration and washed with water. The solid was slurried in water (1 L) and re-filtered, and the solid was left to dry in the funnel overnight. The solid was then dried in a vacuum oven for 2 hours at 60° C. The resulting solid was triturated with dichloromethane (500 mL), and the suspension was filtered and washed with additional dichloromethane. The solid was air-dried to provide the title compound. MS (ESI) m/e 291.8 (M−H)⁻.

### 2.65.11 (2-iodo-4-nitrophenyl)methanol

A flame-dried 3 L 3-necked flask was charged with Example 2.65.10 (51.9 g) and tetrahydrofuran (700 mL). The solution was cooled in an ice bath to 0.5° C., and borane-tetrahydrofuran complex (443 mL, 1M in THF) was added dropwise (gas evolution) over 50 minutes, reaching a final internal temperature of 1.3° C. The reaction mixture was stirred for 15 minutes, and the ice bath was removed. The reaction left to come to ambient temperature over 30 minutes. A heating mantle was installed, and the reaction was heated to an internal temperature of 65.5° C. for 3 hours, and then allowed to cool to room temperature while stirring overnight. The reaction mixture was cooled in an ice bath to 0° C., and quenched by dropwise addition of methanol (400 mL). After a brief incubation period, the temperature rose quickly to 2.5° C., with gas evolution. After the first 100 mL are added over ˜30 minutes, the addition was no longer exothermic, and the gas evolution ceased. The ice bath was removed, and the mixture was stirred at ambient temperature under nitrogen overnight. The mixture was concentrated to a solid, dissolved in dichloromethane/methanol and adsorbed on to silica gel (˜150 g). The residue was loaded on a plug of silica gel (3000 mL) and eluted with dichloromethane to provide the title compound. MS (DCI) m/e 296.8 (M+NH₄)⁺.

### 2.65.12 (4-amino-2-iodophenyl)methanol

A 5 L flask equipped with a mechanical stirrer, heating mantle controlled by a JKEM temperature probe and condenser was charged with Example 2.65.11 (98.83 g) and ethanol (2 L). The reaction was stirred rapidly, and iron (99 g) was added, followed by a solution of ammonium chloride (20.84 g) in water (500 mL). The reaction was heated over the course of 20 minutes to an internal temperature of 80.3° C., when it began to reflux vigorously. The mantle was dropped until the reflux calmed. Thereafter, the mixture was heated to 80° C. for 1.5 hour. The reaction was filtered hot through a membrane filter, and the iron residue was washed with hot 50% ethyl acetate/methanol (800 mL). The eluent was passed through a diatomaceous earth pad, and the filtrate was concentrated. The residue was partitioned between 50% brine (1500 mL) and ethyl acetate (1500 mL). The layers were separated, and the aqueous layer was extracted with ethyl acetate (400 mL×3). The combined organic layers were dried over sodium sulfate, filtered and concentrated to provide the title compound, which was used without further purification. MS (DCI) m/e 266.9 (M+NH₄)⁺.

### 2.65.13 4-(((tert-butyldimethylsilyl)oxy)methyl)-3-iodoaniline

A 5 L flask with a mechanical stirrer was charged with Example 2.65.12 (88 g) and dichloromethane (2 L). The suspension was cooled in an ice bath to an internal temperature of 2.5° C., and tert-butylchlorodimethylsilane (53.3 g) was added portion-wise over 8 minutes. After 10 minutes, 1H-imidazole (33.7 g) was added portionwise to the cold reaction. The reaction was stirred 90 minutes while the internal temperature rose to 15° C. The reaction mixture was diluted with water (3 L) and dichloromethane (1 L). The layers were separated, and the organic layer was dried over sodium sulfate, filtered, and concentrated to an oil. The residue was purified by silica gel chromatography (1600 g silica gel), eluting a gradient of 0-25% ethyl acetate in heptane, to provide the title compound.

### 2.65.14 (S)-2-((S)-2-((((9H-fluoren-9-yl)methoxy)carbonyl)amino)-3-methylbutanamido)propanoic acid

To a solution of (S)-2-((((9H-fluoren-9-yl)methoxy)carbonyl)amino)-3-methylbutanoic acid (6.5 g) in dimethoxyethane (40 mL) was added (S)-2-aminopropanoic acid (1.393 g) and sodium bicarbonate (1.314 g) in water (40 mL). Tetrahydrofuran (20 mL) was added to aid solubility. The resulting mixture was stirred at room temperature for 16 hours. Aqueous citric acid (15%, 75 mL) was added, and the mixture was extracted with 10% 2-propanol in ethyl acetate (2×100 mL). A precipitate formed in the organic layer. The combined organic layers were washed with water (2×150 mL). The organic layer was concentrated under reduced pressure and then triturated with diethyl ether (80 mL). After brief sonication, the title compound was collected by filtration. MS (ESI) m/e 411 (M+H)⁺.

### 2.65.15 (9H-fluoren-9-yl)methyl ((S)-1-(((S)-1-((4-(((tert-butyldimethylsilyl)oxy)methyl)-3-iodophenyl)amino)-1-oxopropan-2-yl)amino)-3-methyl-1-oxobutan-2-yl)carbamate

A solution of Example 2.65.13 (5.44 g) and Example 2.65.14 (6.15 g) in a mixture of dichloromethane (70 mL) and methanol (35.0 mL) was added ethyl 2-ethoxyquinoline-1(2H)-carboxylate (4.08 g), and the reaction was stirred overnight. The reaction mixture was concentrated and the residue was loaded onto silica gel, eluting with a gradient of 10% to 95% heptane in ethyl acetate followed by 5% methanol in dichloromethane. The product-containing fractions were concentrated, dissolved in 0.2% methanol in dichloromethane (50 mL), loaded onto silica gel and eluted with a gradient of 0.2% to 2% methanol in dichloromethane. The product containing fractions were collected to provide the title compound. MS (ESI) m/e 756.0 (M+H)⁺.

### 2.65.16 (2S,3S,4R,5S,6S)-2-((5-((S)-2-((S)-2-((((9H-fluoren-9-yl)methoxy)carbonyl)amino)-3-methylbutanamido)propanamido)-2-(((tert-butyldimethylsilyl)oxy)methyl)phenyl)ethynyl)-6-(methoxycarbonyl)tetrahydro-2H-pyran-3,4,5-triyl triacetate

A solution of Example 2.65.9 (4.500 g), Example 2.65.15 (6.62 g), copper(I) iodide (0.083 g) and bis(triphenylphosphine)palladium(II) dichloride (0.308 g) were combined in vial and degassed. N,N-dimethylformamide (45 mL) and N-ethyl-N-isopropylpropan-2-amine (4.55 mL) were added, and the reaction vessel was flushed with nitrogen and stirred at room temperature overnight. The reaction was partitioned between water (100 mL) and ethyl acetate (250 mL). The layers were separated, and the organic layer was dried over magnesium sulfate, filtered, and concentrated. The residue was purified by silica gel chromatography, eluting with a gradient of 5% to 95% ethyl acetate in heptane. The product containing fractions were collected, concentrated and purified by silica gel chromatography, eluting with a gradient of 0.25% to 2.5% methanol in dichloromethane to provide the title compound. MS (ESI) m/e 970.4 (M+H)⁺.

### 2.65.17 (2S,3S,4R,5S,6S)-2-(5-((S)-2-((S)-2-((((9H-fluoren-9-yl)methoxy)carbonyl)amino)-3-methylbutanamido)propanamido)-2-(((tert-butyldimethylsilyl)oxy)methyl)phenethyl)-6-(methoxycarbonyl)tetrahydro-2H-pyran-3,4,5-triyl triacetate

Example 2.65.16 (4.7 g) and tetrahydrofuran (95 mL) were added to 5% Pt/C (2.42 g, wet) in a 50 mL pressure bottle and the reaction was shaken for 90 minutes at room temperature under 50 psi of hydrogen. The reaction mixture was filtered and concentrated to provide the title compound. MS (ESI) m/e 974.6 (M+H)⁺.

### 2.65.18 (2S,3S,4R, S,6S)-2-(5-((S)-2-((S)-2-((((9H-fluoren-9-yl)methoxy)carbonyl)amino)-3-methylbutanamido)propanamido)-2-(hydroxymethyl)phenethyl)-6-(methoxycarbonyl)tetrahydro-2H-pyran-3,4,5-triyl triacetate

A solution of Example 2.65.17 (5.4 g) in tetrahydrofuran (7 mL), water (7 mL) and glacial acetic acid (21 mL) was stirred overnight at room temperature. The reaction mixture was diluted with ethyl acetate (200 mL) and was washed with water (100 mL), saturated aqueous NaHCO₃ solution (100 mL), and brine (100 mL), dried over magnesium sulfate, filtered, and concentrated. The residue was purified by silica gel chromatography, eluting with a gradient of 0.5% to 5% methanol in dichloromethane, to provide the title compound. MS (ESI) m/e 860.4 (M+H)⁺.

### 2.65.19 (2S,3S,4R,5S,6S)-2-(5-((S)-2-((S)-2-((((9H-fluoren-9-yl)methoxy)carbonyl)amino)-3-methylbutanamido)propanamido)-2-((((4-nitrophenoxy)carbonyl)oxy)methyl)phenethyl)-6-(methoxycarbonyl)tetrahydro-2H-pyran-3,4,5-triyl triacetate

To a solution of Example 2.65.18 (4.00 g) and bis(4-nitrophenyl) carbonate (2.83 g) in acetonitrile (80 mL) was added N-ethyl-N-isopropylpropan-2-amine (1.22 mL) at room temperature. After stirring overnight, the reaction mixture was concentrated, dissolved in dichloromethane (250 mL) and washed with saturated aqueous NaHCO₃ solution (4×150 mL). The organic layer was dried over magnesium sulfate, filtered, and concentrated. The resulting foam was purified by silica gel chromatography, eluting with a gradient of 5% to 75% ethyl acetate in hexanes to provide the title compound. MS (ESI) m/e 1025.5 (M+H)⁺.

### 2.65.20 3-(1-((3-(2-((((4-((S)-2-((S)-2-amino-3-methylbutanamido)propanamido)-2-(2-((2S,3R,4R,5S,6S)-6-carboxy-3,4,5-trihydroxytetrahydro-2H-pyran-2-yl)ethyl)benzyl)oxy)carbonyl)amino)ethoxy)-5,7-dimethyladamantan-1-yl)methyl)-5-methyl-1H-pyrazol-4-yl)-6-(1-(benzo[d]thiazol-2-ylcarbamoyl)-5,6-dihydroimidazo[1,5-a]pyrazin-7(8H)-yl)picolinic acid

The title compound was prepared by substituting Example 1.4.10 for Example 1.12.10 and Example 2.65.19 for Example 2.11.6 in Example 2.11.7. MS (ESI) m/e 1257 (M−H)⁻.

### 2.65.21 (6S)-2,6-anhydro-6-(2-{2-[({[2-((3-[(4-6-[1-(1,3-benzothiazol-2-ylcarbamoyl)-5,6-dihydroimidazo[1,5-a]pyrazin-7(8H)-yl]-2-carboxypyridin-3-yl)-5-methyl-1H-pyrazol-1-yl)methyl]-5,7-dimethyltricyclo[3.3.1.13,7]dec-1-yl}oxy)ethyl]carbamoyl}oxy)methyl]-5-((N-[(2,5-dioxo-2,5-dihydro-1H-pyrrol-1-yl)acetyl]-L-valyl-L-alanyl)amino)phenyl)ethyl)-L-gulonic acid

The title compound was prepared by substituting Example 2.65.20 for Example 2.9.1 in Example 2.10. ¹H NMR (400 MHz, dimethyl sulfoxide-d₆) δ ppm 9.88 (s, 1H), 8.26 (t, 2H), 8.00 (m, 2H), 7.76 (d, 1H), 7.61 (d, 1H), 7.46 (m, 2H), 7.38-7.30 (m, 3H), 7.21 (d, 1H), 7.15 (d, 1H), 7.07 (s, 2H), 7.04 (t, 1H), 5.12 (s, 2H), 4.97 (s, 2H), 4.39 (m, 1H), 4.28 (m, 2H), 4.22 (m, 2H), 4.12 (s, 2H), 4.09 (m, 2H), 3.84 (s, 2H), 3.58 (m, 4H), 3.33 (m, 4H), 3.18-3.00 (m, 4H), 2.94 (t, 2H), 2.80-2.55 (m, 2H), 2.13 (s, 3H), 2.08-1.91 (m, 2H), 1.56 (m, 1H), 1.39 (s, 2H), 1.30-1.20 (m, 6H), 1.26-0.95 (m, 6H), 0.85 (m, 12H). MS (ESI) m/e 1395 (M−H)⁻.

### 2.66 Synthesis of (6S)-2,6-anhydro-6-[2-(2-[({[2-({3-[(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.13,7]dec-1-yl}oxy)ethyl](2-methoxyethyl)carbamoyl}oxy)methyl]-5-{[N-({(3S,5S)-3-(2,5-dioxo-2,5-dihydro-1H-pyrrol-1-yl)-2-oxo-5-[(2-sulfoethoxy)methyl]pyrrolidin-1-yl}acetyl)-L-valyl-L-alanyl]amino}phenyl)ethyl]-L-gulonic acid (Synthon YG)

2.66.1 (3R,7aS)-3-phenyltetrahydropyrrolo[1,2-c]oxazol-5(3H)-one

A solution of (S)-5-(hydroxymethyl)pyrrolidin-2-one (25 g), benzaldehyde (25.5 g) and para-toluenesulfonic acid monohydrate (0.50 g) in toluene (300 mL) was heated to reflux using a Dean-Stark trap under a drying tube for 16 hours. The reaction was cooled to room temperature, and the solvent was decanted from the insoluble materials. The organic layer was washed with saturated aqueous sodium bicarbonate solution (2×) and brine (1×). The organic layer was dried over sodium sulfate, filtered and concentrated under reduced pressure. The residue was purified by flash chromatography on silica gel, eluting with 35/65 heptane/ethyl acetate, to provide the title compound. MS (DCI) m/e 204.0 (M+H)⁺.

### 2.66.2 (3R,6R,7aS)-6-bromo-3-phenyltetrahydropyrrolo[1,2-c]oxazol-5(3H)-one

To a cold (−77° C.) solution of Example 2.66.1 (44.6 g) in tetrahydrofuran (670 mL) was added lithium bis(trimethylsilyl)amide (1.0M in hexanes) (250 mL) dropwise over 40 minutes, keeping Tᵣₓₙ<−73° C. The reaction mixture was stirred at −77° C. for 2 hours, and bromine (12.5 mL) was added dropwise over 20 minutes, keeping Tᵣₓₙ<−64° C. The reaction mixture was stirred at −77° C. for 75 minutes and was quenched by the addition of 150 mL cold 10% aqueous sodium thiosulfate solution to the −77° C. reaction. The reaction mixture was warmed to room temperature and partitioned between half-saturated aqueous ammonium chloride solution and ethyl acetate. The layers were separated, and the organic was washed with water and brine, dried over sodium sulfate, filtered and concentrated under reduced pressure. The residue was purified by silica gel chromatography, eluting with a gradient of 80/20, 75/25, and 70/30 heptane/ethyl acetate to provide the title compound. MS (DCI) m/e 299.0 and 301.0 (M+NH₃+H)⁺.

### 2.66.3 (3R,6S,7aS)-6-bromo-3-phenyltetrahydropyrrolo[1,2-c]oxazol-5(3H)-one

The title compound was isolated as a by-product during the synthesis of Example 2.66.2. MS (DCI) m/e 299.0 and 301.0 (M+NH₃+H)⁺.

### 2.66.4 (3R,6S,7aS)-6-azido-3-phenyltetrahydropyrrolo[1,2-c]oxazol-5(3H)-one

To a solution of Example 2.66.2 (19.3 g) in N,N-dimethylformamide (100 mL) was added sodium azide (13.5 g). The reaction mixture was heated to 60° C. for 2.5 hours. The reaction mixture was cooled to room temperature and quenched by the addition of water (500 mL) and ethyl acetate (200 mL). The layers were separated, and the organic layer was washed brine. The combined aqueous layers were back-extracted with ethyl acetate (50 mL). The combined organic layers were dried with sodium sulfate, filtered and concentrated under reduced pressure. The residue was purified by silica gel chromatography, eluting with 78/22 heptane/ethyl acetate, to provide the title compound. MS (DCI) m/e 262.0 (M+NH₃+H)⁺.

### 2.66.5 (3R,6S,7aS)-6-amino-3-phenyltetrahydropyrrolo[1,2-c]oxazol-5(3H)-one

To a solution of Example 2.66.4 (13.5 g) in tetrahydrofuran (500 mL) and water (50 mL) was added polymer-supported triphenylphosphine (55 g). The reaction was mechanically stirred overnight at room temperature. The reaction mixture was filtered through diatomaceous earth, eluting with ethyl acetate and toluene. The solution was concentrated under reduced pressure, dissolved in dichloromethane (100 mL), dried with sodium sulfate, then filtered and concentrated to provide the title compound, which was used in the subsequent step without further purification. MS (DCI) m/e 219.0 (M+H)⁺.

### 2.66.6 (3R,6S,7aS)-6-(dibenzylamino)-3-phenyltetrahydropyrrolo[1,2-c]oxazol-5(3H)-one

To a solution of Example 2.66.5 (11.3 g) in N,N-dimethylformamide (100 mL) was added potassium carbonate (7.0 g), potassium iodide (4.2 g), and benzyl bromide (14.5 mL). The reaction was stirred at room temperature overnight and quenched by the addition of water and ethyl acetate.

The layers were separated, and the organic layer was washed brine. The combined aqueous layers were back-extracted with ethyl acetate. The combined organic layers were dried with sodium sulfate, filtered and concentrated under reduced pressure. The residue was purified by silica gel chromatography, eluting with a gradient of 10 to 15% ethyl acetate in heptane to give a solid that was triturated with heptane to provide the title compound. MS (DCI) m/e 399.1 (M+H)⁺.

### 2.66.7 (3S,5S)-3-(dibenzylamino)-5-(hydroxymethyl)pyrrolidin-2-one

To a solution of Example 2.66.6 (13 g) in tetrahydrofuran (130 mL) was added para-toluene sulfonic acid monohydrate (12.4 g) and water (50 mL), and the reaction was heated to 65° C. for 6 days. The reaction mixture was cooled to room temperature and was quenched by the addition of saturated aqueous sodium bicarbonate and ethyl acetate. The layers were separated, and the organic layer was washed with brine. The combined aqueous layers were back-extracted with ethyl acetate. The combined organic layers were dried with sodium sulfate, filtered and concentrated under reduced pressure. The waxy solids were triturated with heptane (150 mL) to provide the title compound. MS (DCI) m/e 311.1 (M+H)⁺.

### 2.66.8 (3S,5S)-5-(((tert-butyldimethylsilyl)oxy)methyl)-3-(dibenzylamino)pyrrolidin-2-one

To a solution of Example 2.66.7 (9.3 g) and 1H-imidazole (2.2 g) in N,N-dimethylformamide was added tert-butylchlorodimethylsilane (11.2 mL, 50 weight % in toluene), and the reaction was stirred overnight. The reaction mixture was quenched by the addition of water and diethyl ether. The layers were separated, and the organic layer was washed with brine. The combined aqueous layers were back-extracted with diethyl ether. The combined organic layers were dried with sodium sulfate, filtered and concentrated under reduced pressure. The residue was purified by silica gel chromatography, eluting with 35% ethyl acetate in heptane, to provide the title compound. MS (DCI) m/e 425.1 (M+H)⁺.

### 2.66.9 tert-butyl 2-((3S,5S)-5-(((tert-butyldimethylsilyl)oxy)methyl)-3-(dibenzylamino)-2-oxopyrrolidin-1-yl)acetate

To a cold (0° C.) solution of Example 2.66.8 (4.5 g) in tetrahydrofuran (45 mL) was added 95% sodium hydride (320 mg) in two portions. The cold solution was stirred for 40 minutes, and tert-butyl 2-bromoacetate (3.2 mL) was added. The reaction mixture was warmed to room temperature and stirred overnight. The reaction mixture was quenched by the addition of water and ethyl acetate. The layers were separated, and the organic layer was washed with brine. The combined aqueous layers were back-extracted with ethyl acetate. The combined organic layers were dried with sodium sulfate, filtered and concentrated under reduced pressure. The residue was purified by silica gel chromatography, eluting with a gradient of 5-12% ethyl acetate in heptane, to provide the title compound. MS (DCI) m/e 539.2 (M+H)⁺.

### 2.66.10 tert-butyl 2-((3S,5S)-3-(dibenzylamino)-5-(hydroxymethyl)-2-oxopyrrolidin-1-yl)acetate

To a solution of Example 2.66.9 (5.3 g) in tetrahydrofuran (25 mL) was added tetrabutylammonium fluoride (11 mL, 1.0M in 95/5 tetrahydrofuran/water). The reaction mixture was stirred at room temperature for one hour and was quenched by the addition of saturated aqueous ammonium chloride solution, water and ethyl acetate. The layers were separated, and the organic layer was washed with brine. The combined aqueous layers were back-extracted with ethyl acetate. The combined organic layers were dried with sodium sulfate, filtered and concentrated under reduced pressure. The residue was purified by silica gel chromatography, eluting with 35% ethyl acetate in heptane, to provide the title compound. MS (DCI) m/e 425.1 (M+H)⁺.

### 2.66.11 tert-butyl 2-((3S,5S)-5-((2-((4-((tert-butyldiphenylsilyl)oxy)-2,2-dimethylbutoxy)sulfonyl)ethoxy)methyl)-3-(dibenzylamino)-2-oxopyrrolidin-1-yl)acetate

To a solution of Example 2.66.10 (4.7 g) in dimethyl sulfoxide (14 mL) was added a solution of 4-((tert-butyldiphenylsilyl)oxy)-2,2-dimethylbutyl ethenesulfonate (14.5 g) in dimethyl sulfoxide (14 mL). Potassium carbonate (2.6 g) and water (28 μL) were added, and the reaction was heated at 60° C. under nitrogen for one day. The reaction was cooled to room temperature, and quenched by the addition of brine solution, water and diethyl ether. The layers were separated, and the organic layer was washed with brine. The combined aqueous layers were back-extracted with diethyl ether. The combined organic layers were dried with sodium sulfate, filtered and concentrated under reduced pressure. The residue was purified by silica gel chromatography, eluting with a gradient of 15-25% ethyl acetate in heptane, to provide the title compound. MS (ESI+) me 871.2 (M+H)⁺.

### 2.66.12 tert-butyl 2-((3S,5S)-3-amino-5-((2-((4-((tert-butyldiphenylsilyl)oxy)-2,2-dimethylbutoxy)sulfonyl)ethoxy)methyl)-2-oxopyrrolidin-1-yl)acetate

Example 2.66.11 (873 mg) was dissolved in ethyl acetate (5 mL) and methanol (15 mL), and palladium hydroxide on carbon, 20% by wt (180 mg) was added. The reaction mixture was stirred under a hydrogen atmosphere (30 psi) at room temperature for 30 hours, then at 50° C. for one hour. The reaction was cooled to room temperature, filtered, and concentrated to give the desired product. MS (ESI+) m/e 691.0 (M+H)⁺.

### 2.66.13 4-(((3S,5S)-1-(2-(tert-butoxy)-2-oxoethyl)-5-((2-((4-((tert-butyldiphenylsilyl)oxy)-2,2-dimethylbutoxy)sulfonyl) eth yl)-2-oxopyrrolidin-3-yl)amino)-4-oxobut-2-enoic acid

Maleic anhydride (100 mg) was dissolved in dichloromethane (0.90 mL), and a solution of Example 2.66.12 (650 mg) in dichloromethane (0.90 mL) was added dropwise, and then heated at 40° C. for 2 hours. The reaction mixture was directly purified by silica gel chromatography, eluting with a gradient of 1.0-2.5% methanol in diclioromethane containing 0.2% acetic acid. After concentrating the product-bearing fractions, toluene (10 mL) was added and the mixture was concentrated again to provide the title compound. MS (ESI−) m/e 787.3 (M−H)⁻.

### 2.66.14 tert-butyl 2-((3S,5S)-5-((2-((4-((tert-butyldiphenylsilyl)oxy)-2,2-dimethylbutoxy)sulfonyl)ethoxy)methyl)-3-(2,5-dioxo-2,5-dihydro-1H-pyrrol-1-yl)-2-oxopyrrolidin-1-yl)acetate

Example 2.66.13 (560 mg) was slurried in toluene (7 mL), and triethylamine (220 μL) and sodium sulfate (525 mg) were added. The reaction mixture was heated at reflux under a nitrogen atmosphere for 6 hours, and the reaction mixture stirred at room temperature overnight. The reaction was filtered, and the solids rinsed with ethyl acetate. The eluent was concentrated under reduced pressure, and the residue was purified by silica gel chromatography, eluting with 45/55 heptane/ethyl acetate, ethyl acetate, and then 97.5/2.5/0.2 dichloromethane/methanol/acetic acid to provide the title compound.

### 2.66.15 2-((3S,5S)-3-(2,5-dioxo-2,5-dihydro-1H-pyrrol-1-yl)-2-oxo-5-((2-sulfoethoxy)methyl)pyrrolidin-1-yl)acetic acid

Example 2.66.14 (1.2 g) was dissolved in trifluoroacetic acid (15 mL) and heated to 65-70° C. under nitrogen overnight. The trifluoroacetic acid was removed under reduced pressure. The residue was dissolved in acetonitrile (2.5 mL) and purified by preparative reverse-phase liquid chromatography on a Luna C18(2) AXIA column (250×50 mm, 10p particle size) using a gradient of 5-75% acetonitrile containing 0.1% trifluoroacetic acid in water over 30 minutes, to provide the title compound. MS (ESI−) m/e 375.2 (M−H)⁻.

### 2.66.16 3-(l-((3-(2-((((4-((S)-2-((S)-2-amino-3-methylbutanamido)propanamido)-2-(2-((2S,3R,4R,5S,6S)-6-carboxy-3,4,5-trihydroxytetrahydro-2H-pyran-2-yl)ethyl)benzyl)oxy)carbonyl)(2-methoxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-5-methoxy-3,4-dihydroisoquinolin-2(1H)-yl)picolinic acid

Example 1.12.10 (75 mg) and Example 2.65.19 (100 mg) were dissolved in N,N-dimethylformamide (0.3 mL), 1-Hydroxybenzotriazole (13 mg) and N-ethyl-N-isopropylpropan-2-amine (50 μL) were added, and the reaction was stirred at room temperature for two hours. The reaction mixture was concentrated under reduced pressure. The residue was dissolved in tetrahydrofuran and methanol (0.3 mL each), and lithium hydroxide hydrate (55 mg) in water (0.6 mL) was added. The reaction mixture was stirred at room temperature for one hour and quenched by the addition of N,N-dimethylformamide/water 1/1 (1.5 mL) with trifluoroacetic acid (0.15 mL). The solution was washed with heptane (1 mL), then purified by reverse-phase chromatography (C18 column), eluting with 20-70% acetonitrile in 0.1% trifluoroacetic acid water, to provide the title compound as a trifluoroacetic acid salt. MS (ESI−) m/e 1355.6 (M−H)⁻.

### 2.66.17 (6S)-2,6-anhydro-6-[2-(2-[({[2-({3-(4-{6-[8-(1,3-benzothiazol-2-ylcarbamoyl)-5-methoxy-3,4-dihydroisoquinolin-2(1H)-yl]-2-carboxypyridin-3-yl}-5-methyl-1H-pyrazol-1-yl)methyl]-5,7-dimethyltricyclo[3.3.1.13,7]dec-1-yl}oxy)ethyl](2-methoxyethyl)carbamoyl}oxy)methyl]-5-{[N-({(3S,5S)-3-(2,5-dioxo-2,5-dihydro-1H-pyrrol-1-yl)-[2-oxo-5-[(2-sulfoethoxy)methyl]pyrrolidin-1-yl}acetyl)-L-valyl-L-alanyl]amino}phenyl)ethyl]-L-gulonic acid

To a solution of Example 2.66.15 (20 mg) in N,N-dimethylformamide (0.2 mL) was added O-(7-azabenzotriazol-1-yl)-N,N,N′,N′-tetramethyluronium hexafluorophosphate (20 mg) and N,N-diisopropylethylamine (18 μL). The reaction mixture was stirred for 3 minutes at room temperature and was then added to a solution of Example 2.66.16 (57 mg) and N,N-diisopropylethylamine (30 μL) in N,N-dimethylformamide (0.7 mL). The reaction mixture was stirred at room temperature for 1 hour and diluted with N,N-dimethylformamide/water 1/1 (1.0 mL). The solution was purified by reverse-phase chromatography (C18 column), eluting with 20-70% acetonitrile in 0.10% trifluoroacetic acid water, to provide the title compound. ¹H NMR (400 MHz, dimethyl sulfoxide-d₆) δ ppm 9.84 (br d, 1H), 8.18 (br d, 1H), 8.04 (m, 1H), 8.01 (d, 1H), 7.77 (dd, 2H), 7.50 (d, 1H), 7.46 (m, 3H), 7.34 (t, 1H), 7.29 (s, 1H), 7.21 (br d, 1H), 7.07 (s, 2H), 7.01 (d, 1H), 6.99 (d, 1H), 5.00 (s, 4H), 4.64 (t, 1H), 4.37 (m, 1H), 4.18 (m, 2H), 4.01 (d, 1H), 3.88 (s, 3H), 3.87 (m, 2H), 3.81 (br d, 2H), 3.73 (br m, 1H), 3.63 (m, 2H), 3.55 (m, 2H), 3.49 (m, 2H), 3.36 (br m, 6H), 3.31 (m, 2H), 3.26 (br m, 2H), 3.19 (m, 2H), 3.14 (m, 1H), 3.10 (br m, 1H), 2.94 (t, 1H), 2.81 (m, 3H), 2.74 (m, 2H), 2.60 (br m, 1H), 2.36 (m, 1H), 2.09 (s, 3H), 2.00 (m, 2H), 1.85 (m, 1H), 1.55 (br m, 1H), 1.40-0.92 (m, 14H), 0.88, 0.86, 0.83, 0.79 (d, d, s, s, total 12H). MS (ESI−) me 1713.7 (M−1).

### 2.67 Synthesis of 8-[2-({[(3-amino-3-oxopropyl){2-[(3-{[4-(6-8-[(1,3-benzothiazol-2-yl)carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.1^{3,7}]decan-1-yl)oxy]ethyl}carbamoyl]oxy)methyl)-5-{[(2S)-2({(2S)-2-[2-(2,5-dioxo-2,5-dihydro-1H-pyrrol-1-yl)acetamido]-3-methylbutanoyl}amino)propanoyl]amino}phenyl]-2,6-anhydro-7,8-dideoxy-L-glycero-L-gulo-octonic acid (Synthon ZT)

### 2.67.1 3-(1-((3-(2-((((4-((S)-2-((S)-2-amino-3-methylbutanamido)propanamido)-2-(2-((3R,4R,5S,6S)-6-carboxy-3,4,5-trihydroxytetrahydro-2H-pyran-2-yl)ethyl)benzyl)oxy)carbonyl)(3-amino-3-oxopropyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid

To a cold (0° C.) solution of Example 2.65.19 (66 mg) and Example 1.32.2 (60 mg) in N,N-dimethylformamide (6 mL) was added N,N-diisopropylethylamine (0.026 mL) and 1-hydroxybenzotriazole hydrate (16.23 mg). The reaction mixture was slowly warmed to room temperature and stirred overnight. To the reaction mixture was added water (1 mL) and LiOH H₂O (20 mg). The mixture was stirred at room temperature for 3 hours. The mixture was acidified with trifluoroacetic acid, filtered and purified by reverse-phase HPLC on a Gilson system (C18 column), eluting with 20-80% acetonitrile in water containing 0.1% trifluoroacetic acid, to provide the title compound. MS (ESI) m/e 1338.5 (M−H)⁻.

### 2.67.2 8-[2-({[(3-amino-3-oxopropyl){2-[(3-{([4-(6-8-[(1,3-benzothiazol-2-yl)carbamoyl]-3,4-dihydroisoquinolin-2(1H)-yl}-2-carboxypyridin-3-yl)-5-methyl-1H-pyrazol-1-yl]methyl)-5,7-dimethyltricyclo[3.3.1.13,7]decan-1-yl)oxy]ethyl)}carbamoyl]oxy}methyl)-5-{[(2S)-2-({(2S)-2-[2-(2,5-dioxo-2,5-dihydro-1H-pyrrol-1-yl)acetamido]-3-methylbutanoyl}amino)propanoyl]amino}phenyl]-2,6-anhydro-7,8-dideoxy-L-glycero-L-gulo-octonic acid

The title compound was prepared as described in Example 2.58.7, substituting Example 2.58.6 with Example 2.67.1. ¹H NMR (501 MHz, dimethyl sulfoxide-d₆) δ ppm 9.91 (d, 1H), 8.25 (dd, 2H), 8.03 (d, 1H), 7.79 (d, 1H), 7.61 (d, 6H), 7.55-7.30 (m, 7H), 7.28 (s, 1H), 7.22 (d, 1H), 7.07 (s, 2H), 6.94 (d, 1H), 6.89-6.74 (m, 1H), 5.01 (s, 3H), 4.96 (s, 2H), 4.38 (t, 1H), 4.27-4.17 (m, 1H), 4.12 (d, 2H), 3.88 (t, 2H), 3.79 (d, 1H), 3.41-3.30 (m, 3H), 3.24 (s, 2H), 3.12 (dt, 2H), 3.01 (t, 2H), 2.94 (t, 1H), 2.74 (d, 1H), 2.67-2.56 (m, 1H), 2.29 (t, 2H), 2.08 (d, 3H), 1.99 (d, 3H), 1.55 (d, 1H), 1.42-0.99 (m, 15H), 0.99-0.70 (m, 12H). MS (ESI) m/e 1477.2 (M+H)⁺.

### 2.68 Synthesis of 4-{[({2-[(3-{[4-(6-(8-[(1,3-benzothiazol-2-yl)carbamoyl]-3,4-dihydroisoquinolin-2(1H)-yl)-2-carboxypyridin-3-yl)-5-methyl-1H-pyrazol-1-yl]methyl}-5,7-dimethytricyclo[3.3.1.13,7]decan-1-yl)oxy]ethyl) [3-(methylamino)-3-oxopropyl]carbamoyl)oxy]methyl}-3-3-[2-(2,5-dioxo-2,5-dihydro-1H-pyrrol-1-yl)acetamido]propoxy}phenyl beta-D-glucopyranosiduronic acid (Synthon AAN)

### 2.68.1 3-(1-((3-(2-((((2-(3-aminopropoxy)-4-(((2S,3R,4S,5S,6S)-6-carboxy-3,4,5-trihydroxytetrahydro-2H-pyran-2-yl)oxy)benzyl)oxy)carbonyl)(3-(methylamino)-3-oxopropyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquino-2(1H)-yl)picolinic acid

To a cold (0° C.) solution of Example 2.28.3 (38.7 mg) and Example 1.39 (39.3 mg) in N,N-dimethylformamide (6 mL) was added N,N-diisopropylethylamine (0.026 mL) and 1-hydroxybenzotriazole hydrate (6.58 mg). The reaction was slowly warmed to room temperature and stirred overnight. To the reaction was added water (2 mL) and LiOH H₂O (50 mg), and the mixture was stirred at room temperature for 3 hours. The mixture was acidified with trifluoroacetic acid, filtered and purified by reverse-phase HPLC on a Gilson system (C18 column), eluting with 20-80% acetonitrile in water containing 0.1% trifluoroacetic acid, to provide the title compound. MS (ESI) m/e 1230.2 (M−H)⁻.

### 2.68.2 4-{[({2-[(3-{[4-(6-(8-[(1,3-benzothiazol-2-yl)carbamoyl]-3,4-dihydroisoquinolin-2(1H)-yl}-2-carboxypyridin-3-yl)-5-methyl-1H-pyrazol-1-yl]methyl)-5,7-dimethyltricyclo[3.3.1.13,7]decan-1-yl)oxy]ethyl)} [3-(methylamino)-3-oxopropyl]carbamoyl)oxy]methyl)-3-(3-[2-(2,5-dioxo-2,5-dihydro-1H-pyrrol-1-yl)acetamido]propoxy}phenyl beta-D-glucopyranosiduronic acid

The title compound was prepared as described in Example 2.58.7, substituting Example 2.58.6 with Example 2.68.1 ¹H NMR (501 MHz, dimethyl sulfoxide-d₆) δ ppm 12.88 (s, 2H), 9.93 (d, 1H), 8.36-8.22 (m, 2H), 8.04 (d, 1H), 7.80 (d, 2H), 7.76 (d, OH), 7.62 (d, 1H), 7.56-7.42 (m, 5H), 7.41-7.33 (m, 3H), 7.28 (s, 1H), 7.22 (d, 1H), 7.08 (s, 2H), 6.95 (d, 1H), 5.01 (d, 3H), 4.96 (s, 2H), 4.39 (p, 1H), 4.22 (dd, 1H), 4.12 (d, 2H), 3.89 (t, 2H), 3.80 (d, 2H), 3.34 (t, 2H), 3.22 (d, 2H), 3.13 (dt, 2H), 3.02 (t, 2H), 2.94 (t, 1H), 2.86-2.71 (m, 1H), 2.60 (s, 2H), 2.54 (d, 4H), 2.29 (q, 2H), 2.09 (d, 3H), 2.07-1.90 (m, 3H), 1.60-1.48 (m, 1H), 1.39-1.00 (m, 17H), 0.97-0.74 (m, 15H). (ESI) m/e 1489.5 (M−H)⁻.

### 2.69 Synthesis of 2,6-anhydro-8-(2-{[({2-[(3-{[4-(6-{8-[(1,3-benzothiazol-2-yl)carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.13,7]decan-1-yl)oxy]ethyl}[3-(methylamino)-3-oxopropyl]carbamoyl)oxy]methyl}-5-{[(2S-2-({(2S)-2-[2-(2,5-dioxo-2,5-dihydro-1H-pyrrol-1-yl)acetamido]-3-methylbutanoyl}amino)propanoyl]amino}phenyl)-7,8-dideoxy-L-glycero-L-gulo-octonic acid (Synthon AAO)

### 2.69.1 3(1-((3-(2-((((4-((S)-2-((S)-2-amino-3-methylbutanamido)propanamido)-2-(2-((2,3R,4R,5S,6S)-6-carboxy-3,4,5-trihydroxytetrahydro-2H-pyran-2-yl)ethyl)benzyl)oxy)carbonyl)(3-(methylamino)-3-oxopropyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid

The title compound was prepared as described in Example 2.67.1, substituting Example 1.32.2 with Example 1.39. MS (ESI) n/e 1352.6 (M−H)⁻.

### 2.69.2 2,6-anhydro-8-(2-{[({2-[(3-{[4-(6-{8-[(1,3-benzothiazol-2-yl)carbamoyl]-3,4-dihydroisoquinolin-2(1H)-yl}-2-carboxypyridin-3-yl-5-methyl-1H-pyrazol-1-yl]methyl}-5,7-dimethyltricyclo[3.3.1.13,7]decan-1-yl)oxy]ethyl}[3-(methylamino)-3-oxopropyl]carbamoyl)oxy]methyl}-5-{[(2S)-2-({(2S)-2-[2-(2,5-dioxo-2,5-dihydro-1H-pyrrol-1-yl)acetamido]-3-methylbutanoyl}amino)propanoyl]amino}phenyl)-7,8-dideoxy-L-glycero-L-gulo-octonic acid

The title compound was prepared as described in Example 2.58.7, substituting Example 2.58.6 with Example 2.67.1. ¹H NMR (501 MHz, dimethyl sulfoxide-d₆) δ ppm 12.88 (s, 2H), 9.93 (d, 1H), 8.36-8.22 (m, 2H), 8.04 (d, 1H), 7.80 (d, 2H), 7.76 (d, OH), 7.62 (d, 1H), 7.56-7.42 (in, 5H), 7.41-7.33 (m, 3H), 7.28 (s, 1H), 7.22 (d, 1H), 7.08 (s, 2H), 6.95 (d, 1H), 5.01 (d, 3H), 4.96 (s, 2H), 4.39 (p, 1H), 4.22 (dd, 1H), 4.12 (d, 2H), 3.89 (t, 2H), 3.80 (d, 2H), 3.34 (t, 2H), 3.22 (d, 2H), 3.13 (dt, 2H), 3.02 (t, 2H), 2.94 (t, 1H), 2.86-2.71 (m, 1H), 2.60 (s, 2H), 2.54 (d, 4H), 2.29 (q, 2H), 2.09 (d, 3H), 2.07-1.90 (m, 3H), 1.60-1.48 (m, 1H), 1.39-1.00 (m, 17H), 0.97-0.74 (m, 15H). MS (ESI) m/e 1489.5 (M−H)⁻.

### 2.70 Synthesis of 2,6-anhydro-8-(2-{[({2-[(3-{[4-(6-{8-[(1,3-benzothiazol-2-yl)carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.13,7]decan-1-yl)oxy]ethyl}[3-(methylamino)-3-oxopropyl]carbamoyl)oxy]methyl}-5-{[(2S)-2-{[(2S)-2-(2-{(3S,5S)-3-(2,5-dioxo-2,5-dihydro-1H-pyrrol-1-yl)-2-oxo-5-[(2-sulfoethoxy)methyl]pyrrolidin-1-yl}acetamido)-3-methylbutanoyl]amino}propanoyl]amino}phenyl)-7,8-dideoxy-L-glycero-L-gulo-octonic acid (Synthon AAP)

To a solution of Example 2.66.15 (17 mg) in N,N-dimethylformamide (320 μL) was added O-(7-azabenzotriazol-1-yl)-N,N,N′,N′-tetramethyluronium hexafluorophosphate (19 mg) and N,N-diisopropylethylamine (17 μL). The reaction mixture was stirred for 5 minutes and was added to a solution of Example 2.69.1 (39 mg) and N,N-diisopropylethylamine (36 μL) in N,N-dimethylformamide (320 μL). The reaction mixture was stirred for 2 hours and was diluted with N,N-dimethylformamide (2 mL). The solution was filtered and purified by reverse-phase HPLC on a Gilson system (C18 column), eluting with 20-80% acetonitrile in water containing 0.1% trifluoroacetic acid, to provide the title compound. ¹H NMR (501 MHz, dimethyl sulfoxide-d₆) δ ppm 9.82 (s, 1H), 8.15 (d, 1H), 8.00 (dd, 2H), 7.75 (d, 1H), 7.58 (d, 1H), 7.44 (ddd, 5H), 7.32 (td, 2H), 7.25 (s, 1H), 7.18 (d, 1H), 7.03 (s, 2H), 6.92 (d, 1H), 6.76 (s, 1H), 4.97 (s, 2H), 4.92 (s, 2H), 4.61 (t, 1H), 4.33 (p, 1H), 4.21-4.08 (m, 2H), 3.98 (d, 1H), 3.84 (t, 2H), 3.40-3.27 (m, 3H), 3.21 (s, 1H), 3.14-3.03 (m, 2H), 2.98 (t, 2H), 2.90 (t, 1H), 2.81-2.50 (m, 4H), 2.38-2.20 (m, 3H), 2.05 (s, 3H), 2.01-1.90) (m, 2H), 1.88-1.74 (m, 1H), 1.60-1.43 (m, 1H), 1.36-0.95 (m, 14H), 0.95-0.62 (m, 13H). MS (ESI) m/e 1710.5(M−H)⁻.

### 2.71 Synthesis of 6-{8-[(1,3-benzothiazol-2-yl)carbamoyl]-3,4-dihydroisoquinolin-2(1H)-yl}-3-[1-({3-[2-({[(4-{(2S)-5-(carbamoylamino)-2-{[(2S)-2-{[6-(2,5-dioxo-2,5-dihydro-1H-pyrrol-1-yl)hexanoyl]amino}-3-methylbutanoyl]amino) pentanoyl] amino}phenyl)methoxy]carbonyl}ami no)acetamido]-5,7-dimethyltricyclo[3.3.1.13,7]decan-1-yl}methyl)-5-methyl-1H-pyrazol-4-yl]pyridine-2-carboxylic acid (Synthon ABF)

The title compound was prepared as described in Example 2.2, substituting Example 1.3.2 with Example 1.40.11. ¹H NMR (501 MHz, dimethyl sulfoxide-d₆) δ ppm 9.96 (s, 1H), 8.03 (dd, 2H), 7.78 (d, 2H), 7.59 (dd, 3H), 7.53-7.39 (m, 3H), 7.35 (q, 2H), 7.30-7.23 (m, 3H), 7.20 (d, 1H), 6.98 (s, 2H), 6.94 (d, 1H), 4.94 (d, 4H), 4.38 (t, 1H), 4.17 (dd, 1H), 3.87 (t, 2H), 3.78 (s, 2H), 3.35 (t, 2H), 3.00 (t, 3H), 2.94 (s, OH), 2.16 (d, 1H), 2.09 (s, 3H), 1.95 (d, 1H), 1.74-1.27 (m, 10H), 1.13 (dq, 5H), 0.87-0.71 (m, 12H). MS (ESI) m/e 1355.5(M−H)⁻.

### 2.72 Synthesis of 8-[2-({[(3-amino-3-oxopropyl){2-[(3-{[4-(6-8-[(1,3-benzothiazol-2-yl)carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.13,7]decan-1-yl)oxy]ethyl}carbamoyl]oxy)methyl)-5-{[(2S)-2-{[(2S)-2-(2-((3S,5S)-3-(2,5-dioxo-2,5-dihydro-1H-pyrrol-1-yl)-2-oxo-5-[(2-sulfoethoxy)methyl]pyrrolidin-1-yl)acetamido)-3-methylbutanoyl] amino}propanoyl]amino}phenyl]-2,6-anhydro-7,8-dideoxy-L-glycero-L-gulo-octonic acid (Synthon ZZ)

### 2.72.1 3-(1-((3-(2-((((4-((S)-2-((S)-2-amino-3-methylbutanamido)propanamido)-2-(2-((3R,4R,5S,6S)-6-carboxy-3,4,5-trihydroxytetrahydro-2H-pyran-2-yl)ethyl)benzyl)oxy)carbonyl)(3-amino-3-oxopropyl)amino)ethoxy)-5,7-dimethyladamnantan-1-yl)methyl)-5-methyl-1H-pyrazol-4-yl)-6-(8-(benzo[d]thiazol-2-ylcarbamoyl)-3,4-dihydroisoquinolin-2(l H)-yl)picolinic acid

To a cold (0° C.) solution of Example 2.65.19 (66 mg) and Example 1.32.2 (6 mL) were added N,N-diisopropylamine (0.026 mL) and 1-hydroxybenzotriazole hydrate (16.23 mg). The reaction mixture was slowly warmed to room temperature and stirred overnight. To the reaction mixture was added water (1 mL) and LiOH H₂O (20 mg), and the mixture was stirred at room temperature for 3 hours. The mixture was acidified with trifluoroacetic acid, filtered and was purified by reverse-phase HPLC on a Gilson system (C18 column), eluting with 20-80% acetonitrile in water containing 0.1% trifluoroacetic acid, to provide the title compound. MS (ESI) m/e 1338.5 (M−H)⁻.

### 2.72.2 8-[2-({[(3-amino-3-oxopropyl){2-[(3-{[4-(6-{8-[(1,3-benzothiazol-2-yl)carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.1^{3,7}]decan-1-yl)oxy]ethyl}carbamoyl]oxy}methyl)-5-{[(2S)-2-{[(2S)-2-(2-{(3S,5S)-3-(2,5-dioxo-2,5-dioxo-2,5-dihydro-1H-pyrrol-1-yl)-2-oxo-5-[(2-sulfoethoxy)methyl]pyrrolidin-1-yl}acetamido)-3-methylbutanoyl]amino}propanoyl]amino}phenyl]-2,6-anhydro-7,8-dideoxy-L-glycero-L-gulo-octonic acid

To a solution of 2-((3S,5S)-3-(2,5-dioxo-2,5-dihydro-1H-pyrrol-1-yl)-2-oxo-5-((2-sulfoethoxy)methyl)pyrrolidin-1-yl)acetic acid (17 mg) in N,N-dimethylformamide (320 μL), was added O-(7-azabenzotriazol-1-yl)-N,N,N′,N′-tetramethyluronium hexafluorophosphate (19 mg) and N-ethyl-N-isopropylpropan-2-amine (17 μL). The reaction mixture was stirred for 5 minutes and was added to a solution of Example 2.72.1 (50 mg) and N-ethyl-N-isopropylpropan-2-amine (36 μL) in N,N-dimethylformamide (320 μL). The reaction mixture was stirred for 2 hours. The reaction mixture was diluted with N,N-dimethylformamide/water (1/1, 1 mL) and purified by reverse-phase HPLC on a Gilson system (C18 column), eluting with 20-80% acetonitrile in water containing 0.1% trifluoroacetic acid, to provide the title compound. ¹H NMR (501 MHz, dimethyl sulfoxide-d₆) δ ppm 9.82 (s, 1H), 8.15 (d, 1H), 8.00 (dd, 2H), 7.75 (d, 1H), 7.58 (d, 1H), 7.44 (ddd, 5H), 7.32 (td, 2H), 7.25 (s, 1H), 7.18 (d, 1H), 7.03 (s, 2H), 6.92 (d, 1H), 6.76 (s, 1H), 4.97 (s, 2H), 4.92 (s, 2H), 4.61 (t, 1H), 4.33 (p, 1H), 4.21-4.08 (m, 2H), 3.98 (d, 1H), 3.84 (t, 2H), 3.40-3.27 (m, 3H), 3.21 (s, 1H), 3.14-3.03 (m, 2H), 2.98 (t, 2H), 2.90 (t, 1H), 2.81-2.50 (m, 4H), 2.38-2.20 (m, 3H), 2.05 (s, 3H), 2.01-1.90 (m, 2H), 1.88-1.74 (m, 1H), 1.60-1.43 (m, 1H), 1.36-0.95 (m, 14H), 0.95-0.62 (m, 13H). MS (ESI) m/e 1697.5 (M−H)⁻.

### 2.73 Synthesis of N-[6-(2,5-dioxo-2,5-dihydro-1H-pyrrol-1-yl)hexanoyl]-L-valyl-N- {4-[({[2-((3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.13,7]dec-1-yl}oxy)ethyl](2-sulfoethyl)carbamoyl}oxy)methyl]phenyl)-N5-carbamoyl-L-ornithinamide (Synthon CZ)

Example 1.44.2 (100 mg) and 4-((S)-2-((S)-2-(6-(2,5-dioxo-2,5-dihydro-1H-pyrrol-1-yl)hexanamido)-3-methylbutanamido)-5-ureidopentanamido)benzyl (4-nitrophenyl) carbonate (purchased from Synchem, 114 mg) in N,N-dimethylformamide (7 mL) was cooled in an water-ice bath, and N,N-diisopropylethylamine (0.15 mL) was added. The mixture was stirred at 0° C. for 30 minutes and then at room temperature overnight. The reaction was purified by a reverse phase HPLC using a Gilson system, eluting with 20-60% acetonitrile in water containing 0.1% v/v trifluoroacetic acid, to provide the title compound. ¹H NMR (400 MHz, dimethylsulfoxide-d₆) δ ppm 12.85 (s, 1H), 9.99 (s, 1H), 8.04 (t, 2H), 7.75-7.82 (m, 2H), 7.40-7.63 (m, 6H), 7.32-7.39 (m, 2H), 7.24-7.29 (m, 3H), 6.99 (s, 2H), 6.95 (d, 1H), 6.01 (s, 1H), 4.83-5.08 (m, 4H), 4.29-4.48 (m, 1H), 4.19 (t, 1H), 3.84-3.94 (m, 2H), 3.80 (d, 2H), 3.14-3.29 (m, 2H), 2.87-3.06 (m, 4H), 2.57-2.69 (m, 2H), 2.03-2.24 (m, 5H), 1.89-2.02 (m, 1H), 1.53-1.78 (m, 2H), 1.26-1.53 (m, 8H), 0.89-1.27 (m, 12H), 0.75-0.88 (m, 12H). MS (ESI) m/e 1452.2 (M+H)⁺.

### 2.74 Synthesis of 6-(8-(benzo[d]thiazol-2-ylcarbamoyl)-3,4-dihydroisoquinolin-2(1H)-yl)-3-(1-((3-(2-((((2-(2-((2S,3R,4R,5S,6S)-6-carboxy-3,4,5-trihydroxytetrahydro-2H-pyran-2-yl)ethyl)-4-((S)-2-((S)-2-(2-((3S,5S)-3-(2,5-dioxo-2,5-dihydro-1H-pyrrol-1-yl)-2-oxo-5-((2-sulfoethoxy)methyl)pyrrolidin-1-yl)acetamido)-3-methylbutanamido)propanamido)benzyl)oxy)carbonyl)(2-sulfoethyl)amino)ethoxy)-5,7-dimethyladamantan-1-yl)meth--et)-methyl-1H-pyrazol-4-yl)picolinic acid (Synthon TX)

### 2.74.1 3-(1-(((1r,3s,5R,7S)-3-(2-((((4-((R)-2-((R)-2-amino-3-methylbutanamido)propanamido)-2-(2-((2S,3R,4R,5S,6S)-6-carboxy-3,4,5-trihydroxytetrahydro-2H-pyran-2-yl)ethyl)benzyl)oxy)carbonyl)(2-sulfoethyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid

To a cold (0° C.) solution of Example 2.65.19 (70 mg) and Example 1.44.2 (58.1 mg) in N,N-dimethylformamide (4 mL) was added N-ethyl-N-isopropylpropan-2-amine (0.026 mL). The reaction was slowly warmed to room temperature and stirred overnight. To the reaction mixture was added water (1 mL) and LiOH H₂O (20 mg). The mixture was stirred at room temperature for 3 hours. The mixture was acidified with trifluoroacetic acid, filtered and purified by reverse-phase HPLC on a Gilson system (C18 column), eluting with 20-80% acetonitrile in water containing 0.1% trifluoroacetic acid, to give the title product.

MS (ESI) m/e 1564.4 (M−H)⁻.

### 2.74.2 6-(8-(benzo[d]thiazol-2-ylcarbamoyl)-3,4-dihydroisoquinolin-2(1H)-yl)-3-(1-((3-(2-((((2-(2-((2S,3R,4R,5S,6S)-6-carboxy-3,4,5-trihydroxytetrahydro-2H-pyran-2-yl)ethyl)-4-((S)-2-((S)-2-(2-((3S,5S)-3-(2,5-dioxo-2,5-dihydro-1H-pyrrol-1-yl)-2-oxo-5-((2-sulfoethoxy)methyl)pyrrolidin-1-yl)acetamido)-3-methylbutanamido)propanamido)benzyl)oxy)carbonyl)(2-sulfoethyl)amino)ethoxy)-5,7-dimethyladamantan-1-yl)methyl)-5-methyl-1H-pyrazol-4-yl)picolinic acid

The title compound was prepared by substituting Example 2.74.1 for Example 2.66.16 in Example 2.66.17. ¹H NMR (400 MHz, dimethylsulfoxide-d₆) δ ppm 9.85 (s, 1H), 8.17 (br d, 1H), 8.01 (d, 2H), 7.77 (d, 1H), 7.59 (d, 1H), 7.53 (d, 1H), 7.43 (m, 4H), 7.34 (m, 3H), 7.19 (d, 1H), 7.06 (s, 2H), 6.96 (d, 1H), 4.99 (m, 2H), 4.95 (s, 2H), 4.63 (t, 1H), 4.36 (t, 1H), 4.19 (br m, 1H), 4.16 (d, 1H), 3.98 (d, 1H), 3.87 (br t, 2H), 3.81 (br d, 2H), 3.73 (brm, 1H), 3.63 (t, 2H), 3.53 (m, 2H), 3.44 (m, 4H), 3.31 (t, 2H), 3.21 (br m, 2H), 3.17 (m, 2H), 3.00 (m, 2H), 2.92 (br m, 1H), 2.75 (m, 3H), 2.65 (br m, 3H), 2.35 (br m, 1H), 2.07 (s, 3H), 1.98 (br m, 2H), 1.85 (m, 1H), 1.55 (br m, 1H), 1.34 (br m, 1H), 1.26 (br m, 6H), 1.09 (br m, 7H), 0.93 (br m, 1H), 0.87, 0.83, 0.79 (all d, total 12H). MS (ESI) m/e 1733.4 (M−H)⁻.

### Example 3: Generation of Rat and Mouse Anti-CD98 Monoclonal Antibodies by Murine Hybridoma Technology

In order to identify CD98 specific antibodies, hybridoma technology was used to isolate murine monoclonal anti-CD98 antibodies.

Rats and mice were immunized by hock immunizations (Kamala et al., Hock immunization: A humane alternative to mouse footpad injections J Immunol Method 2007, 328: 204-214. Recombinant extracellular domain (ECD) of human CD98 was used as an immunogen. Sera titers were determined by binding to recombinant hCD98-ECD (ELISA) or to MCF7 cells (Flow Cytometry). Immunizing dosages each contained 20 μg of recombinant hCD98-ECD (Table 1) for both primary and boost immunizations. GerbuMM adjuvant (GERBU Biotechnik GmbH Cat #3001.6001) was mixed with antigen to induce immune response. Briefly, 20 μg of antigen was diluted in PBS and mixed with an equal volume of adjuvant by robust vortexing. The adjuvant-antigen solution in a volume of 20-25 μl was drawn into the proper syringe for animal injection and was injected at mouse leg hock. Each animal received a primary immunization followed by and boosts every three days for total of 5 to 6 immunizations.

TABLE 1
Amino Acid Sequences of Recombinant CD98 Extracellular Domain (ECD)
of Human and Cynomolgus Monkey Used in Hybridoma Generation
and Screening

| | | |
|---|---|---|
| Human CD98 ECD | SEQ ID | GGSGGHHHHHHRAPRCRELPAQKWWHTGALYRIGDLQA |
| with N- | NO: 126 | FQGHGAGNLAGLKGRLDYLSSLKVKGLVLGPIHKNQKD |
| terminal His- | | DVAQTDLLQIDPNFGSKEDFDSLLQSAKKKSIRVILDL |
| tag | | TPNYRGENSWFSTQVDTVATKVKDALEFWLQAGVDGFQ |
| | | VRDIENLKDASSFLAEWQNITKGFSEDRLLIAGTNSSD |
| | | LQQILSLLESNKDLLLTSSYLSDSGSTGEHTKSLVTQY |
| | | LNATGNRWCSWSLSQARLLTSFLPAQLLRLYQLMLFTL |
| | | PGTPVFSYGDEIGLDAAALPGQPMEAPVMLWDESSFPD |
| | | IPGAVSANMTVKGQSEDPGSLLSLFRRLSDQRSKERSL |
| | | LHGDFHAFSAGPGLFSYIRHWDQNERFLVVLNFGDVGL |
| | | SAGLQASDLPASASLPAKADLLLSTQPGREEGSPLELE |
| | | RLKLEPHEGLLLRFPYAAAAA |
| Cynomolgus | SEQ ID | RAPRCRELPAQKWWHTGALYRIGDLQAFQGHGSGNLAG |
| monkey CD98 | NO: 127 | LKGRLDYLSSLKVKGLVLGPLHKNQKDDVAQTDLLQID |
| ECD with C- | | PNFGSKEDFDNLLQSAKKKSIRVILDLTPNYRGENLWF |
| terminal His- | | STQVDSVATKVKDALEFWLQAGVDGFQVRDIENLKDAS |
| tag | | SFLAEWENITKGFSEDRLLIAGTNSSDLQQIVSLLESN |
| | | KDLLLTSSYLSDSSFTGEHTKSLVTQYLNATGNRWCSW |
| | | SLSQAGLLTSFLPAQLLRLYQLMLFTLPGTPVFSYGDE |
| | | IGLKAAALPGQPVEAPVMLWDESSFPDIPGAVSANMTV |
| | | KGQSEDPGSLLSLFRQLSDQRSKERSLLHGDFHTFSSG |
| | | PGLFSYIRHWDQNERFLVVLNFGDVGLSAGLQASDLPA |
| | | SASLPTKADLVLSTQPGREEGSPLELERLKLEPHEGLL |
| | | LRFPYVAAAAHHHHHH |

Note:
Polvhistidine-tag and linker sequences arc underlined and bold.

### Hybridoma Fusion and Screening.

Cells of murine myeloma cell line (NSO-Mouse Myeloma. PTA-4796) were cultured to reach the log phase stage prior to fusion. Lymph node cells were isolated from immunized animals and enriched for IgG producing cells using RoboSep. Enriched cells were fused with myeloma cells using an electrofusion technique (see WO2014/093786). Fused “hybrid cells” were dispensed into 96-well plates and cultured in selective media. Surviving hybridoma colonies were observed macroscopically seven to ten days post-fusion. Once colonies had reached sufficient size, seven to ten days post-fusion, the supernatant from each well was tested by ELISA-based screening using recombinant human and cynomolgus monkey CD98-ECD (Table 1).

ELISA plates were coated with human or cynomolgus monkey CD98-ECD at 2 μg/ml in Carbonate/Bicarbonate buffer at 4° C. overnight, blocked with 2% milk in PBS for one hour at room temperature, washed three times with PBS+0.05% Tween-20 (PBST). Hybridoma supernatants diluted 1:3 in PBS+0.1% BSA (bovine serum albumin) were added to the plates and incubated for one hour at room temperature. ELISA plates were washed three times with PBST. Goat anti-mouse (or anti-rat) IgG conjugated to HRP (horse radish peroxidase) diluted 1:5000 in PBST+10% Superblock; 50 μL/well was added to the plates and incubated for one hour at room temperature. Plates were washed three times with PBST. TMB solution (InVitrogen) was added to each well, 50 μL/well, at room temperature. The reaction was stopped by the addition of hydrochloric acid. Plates were read spectrophotometrically at a wavelength of 450 nm.

Selected supernatants from positive hybridoma hits were tested for binding to cell surface human or cynomolgus monkey CD98. Two cell lines were used for flow cytometry based screening: MCF7 cells endogenously expressing human CD98 and 3T12 cells stably transfected to express cynomolgus monkey CD98.

Screening cell lines were dispensed into 96-well (round bottom) plates at 1×10⁶ cells/well and incubated with diluted hybridoma supernatant at 4° C. for 20 min. Cells were then washed three times with FACS buffer (PBS+2% FBS). Goat anti-mouse (or anti-rat) Ig-PE (phycoerythrin) was used for detection. Hybridomas secreting antibody which bound to either human or cyno cell surface CD98 were transferred to 24-well plates and subcloned by single cell sort to ensure the clonality of the cell line. The isotype of each monoclonal antibody was determined using the BD Pharmingen Rat Immunoglobulin Isotyping ELISA Kit (Cat: 557081) or Thermo Scientific Pierce Rapid ELISA Mouse mAb Isotyping Kit

### (Cat: 37503).

Hybridoma clones producing antibodies that showed high specific binding activity were subcloned, scaled up and purified for further characterization. In total, five mouse anti-CD98 hybridoma mAbs (Ab1-Ab5) and ten rat anti-CD98 hybridoma mAbs (Ab6-Ab15) were selected for further study (Table 2).

TABLE 2
Anti-CD98 Murine Hybridoma Antibodies

| | | | Heavy | Light |
|---|---|---|---|---|
| Hybridoma | | | Chain | Chain |
| Name | Species | Isotype | MW (Da)* | MW (Da) |
| Ab1 | Mouse | IgG1 Kappa | 50671.88 | 24469.49 |
| Ab2 | Mouse | IgG1 Kappa | 52708.74 | 24484.37 |
| Ab3 | Mouse | IgG1 Kappa | 50392.84 | 24457.34 |
| Ab4 | Mouse | IgG1 Kappa | 50674.12 | 24374.32 |
| Ab5 | Mouse | IgG1 Kappa | 50641.84 | 24414.12 |
| Ab6 | Rat | IgG1 Kappa | 52652.76 | 24492.9 |
| Ab7 | Rat | IgG1 Kappa | 51060.64 | 24546.19 |
| Ab8 | Rat | IgG1 Kappa | 49560.96 | 24190.68 |
| Ab9 | Rat | IgG2a Kappa | 50478.33 | 24541.73 |
| Ab10 | Rat | IgG2a Kappa | 50265.94 | 24243.51 |
| Ab11 | Rat | IgG2a Kappa | 50277.42 | 24230.57 |
| Ab12 | Rat | IgG2a Kappa | 50307.99 | 24188.45 |
| Ab13 | Rat | IgG2a Kappa | 49492.23 | 23625.91 |
| Ab14 | Rat | IgG1 Kappa | 49461.6 | 24445.9 |
| Ab15 | Rat | IgG1 Kappa | 49710.18 | 24331.81 |

MW = Molecular weight observed in mass spectroscopy;
*= MW of the agalactosylated (G0) heavy chain peak.

### Example 4. Binding Affinity of Anti-CD98 Murine Hybridoma Monoclonal Antibodies

The binding kinetics of these purified mouse and rat monoclonal anti-CD98 antibodies for purified recombinant CD98 protein (extracellular domain. ECD) were determined by surface plasmon resonance-based measurements made on Biacore T100/T200 instruments (GE Healthcare, Piscataway, N.J.) at 25° C. using an anti-Fc capture assay approach. Binding kinetic measurements were made in the assay buffer HBS-EP+: 10 mM Hepes, pH 7.4, 150 mM NaCl, 3 mM EDTA, 0.05% Tween 20). For example, approximately 8000 RU of anti-Fc (species specific) polyclonal antibody (Thermo Fisher Scientific Inc. Rockford, Ill.) diluted in 10 mM sodium acetate (pH 4.5) was directly immobilized across a CM5 research grade biosensor chip using a standard amine coupling kit according to manufacturer's instructions and procedures at 25 μg/ml. Unreacted moieties on the biosensor surface were blocked with ethanolamine. The test antibody to be captured as a ligand was diluted in running buffer to ˜0.5 μg/mL and injected over anti-Fc surface at a flow rate of 10 μL/min, CD98 binding and dissociation were observed under a continuous flow rate of 80 μL/min. Human and cynomolgus monkey CD98 ECDs, both with a C-terminal His-tag, are used for this study (Table 3). After each cycle the anti-Fc capture surface was regenerated using 10 mM Glycine-HCl, pH 1.5. During the assay, all measurements were referenced against the capture surface alone (i.e., with no captured test antibody) and buffer-only injections (no antigen) were used for double referencing. For kinetic analysis, rate equations derived from the 1:1 Langmuir binding model were fitted simultaneously, globally and with mass transfer term included to multiple referenced antigen binding curves using Biacore T100T200 Evaluation software. The association and dissociation rate constants for CD98 binding, kₐ(M⁻¹s⁻¹) and k_{d} (s⁻¹), and the equilibrium dissociation constant K_{D} (M) of the interaction between antibodies and the target antigen were derived by making kinetic binding measurements at different antigen concentrations ranging from 3.7-900 nM, as a 3-fold dilution series. Results are shown in Table 4 below.

TABLE 3
Amino Acid Sequences of Recombinant CD98 Extracellular Domain
(ECD for Binding Affinity Determination

| | | |
|---|---|---|
| Human CD98 ECD | SEQ ID | RAPRCRELPAQKWWHTGALYRIGDLQAFQGHGAGNLAG |
| with C- | NO: 128 | LKGRLDYLSSLKVKGLVLGPIHKNQKDDVAQTDLLQID |
| terminal His- | | PNFGSKEDFDSLLQSAKKKSIRVILDLTPNYRGENSWF |
| tag | | STQVDTVATKVKDALEFWLQAGVDGFQVRDIENLKDAS |
| | | SFLAEWQNITKGFSEDRLLIAGTNSSDLQQILSLLESN |
| | | KDLLLTSSYLSDSGSTGEHTKSLVTQYLNATGNRWCSW |
| | | SLSQARLLTSFLPAQLLRLYQLMLFTLPGTPVFSYGDE |
| | | IGLDAAALPGQPMEAPVMLWDESSFPDIPGAVSANMTV |
| | | KGQSEDPGSLLSLFRRLSDQRSKERSLLHGDFHAFSAG |
| | | PGLFSYIRHWDQNERFLWLNFGDVGLSAGLQASDLPA |
| | | SASLPAKADLLLSTQPGREEGSPLELERLKLEPHEGLL |
| | | LRFPYAAAAAHHHHHH |
| Cynomolgus | SEQ ID | RAPRCRELPAQKWWHTGALYRIGDLQAFQGHGSGNLAG |
| monkey CD98 | NO: 129 | LKGRLDYLSSLKVKGLVLGPLHKNQKDDVAQTDLLQID |
| ECD with C- | | PNFGSKEDFDNLLQSAKKKSIRVILDLTPNYRGENLWF |
| terminal His- | | STQVDSVATKVKDALEFWLQAGVDGFQVRDIENLKDAS |
| tag | | SFLAEWENITKGFSEDRLLIAGTNSSDLQQIVSLLESN |
| | | KDLLLTSSYLSDSSFTGEHTKSLVTQYLNATGNRWCSW |
| | | SLSQAGLLTSFLPAQLLRLYQLMLFTLPGTPVFSYGDE |
| | | IGLKAAALPGQPVEAPVMLWDESSFPDIPGAVSANMTV |
| | | KGQSEDPGSLLSLFRQLSDQRSKERSLLHGDFHTFSSG |
| | | PGLFSYIRHWDQNERFLWLNFGDVGLSAGLQASDLPA |
| | | SASLPTKADLVLSTQPGREEGSPLELERLKLEPHEGLL |
| | | LRFPYVAAAAHHHHHH |

Note:
Polyhistidine-tag and linker sequences arc underlined and bold.

TABLE 4
Biacore Kinetics of Anti-CD98 Murine Hybridoma Antibodies
Binding to Human and Cynomolgus Monkey CD98

| | Kinetics on Biacore | | | | | |
|---|---|---|---|---|---|---|
| Anti-CD98 | huCD98 ECD | | | cyCD98 ECD | | |
| Hybridoma | kₐ | k_{d} | K_{D} | kₐ | k_{d} | K_{D} |
| mAb | (M⁻¹s⁻¹) | (s⁻¹) | (M) | (M⁻¹s⁻¹) | (s⁻¹) | (M) |
| Ab1 | 8.5E+04 | ≤5.0E−06* | ≤5.9E−11 | 4.3E+04 | ≤5.0E−06* | ≤1.2E−10 |
| Ab2 | 7.3E+04 | 8.1E−05 | 1.1E−09 | 2.4E+04 | 8.6E−04 | 3.7E−08 |
| Ab3 | 2.0E+05 | 3.0E−03 | 1.5E−08 | 3.8E+04 | 1.2E−03 | 3.0E−08 |
| Ab4 | 4.4E+04 | 3.7E−05 | 8.3E−10 | 9.7E+03 | 1.7E−05 | 1.8E−09 |
| Ab5 | 5.2E+04 | 1.2E−04 | 2.2E−09 | 2.1E+04 | 2.2E−04 | 1.1E−08 |
| Ab6 | 1.5E+05 | 1.6E−04 | 1.1E−09 | 6.2E+04 | 3.7E−04 | 5.9E−09 |
| Ab7 | 2.0E+05 | 1.5E−04 | 7.6E−10 | 9.1E+04 | 2.7E−04 | 3.0E−09 |
| Ab8 | 8.5E+04 | 1.2E−04 | 1.4E−09 | 3.6E+04 | 1.3E−04 | 3.7E−09 |
| Ab9 | 1.3E+05 | 1.5E−04 | 1.2E−09 | 5.1E+04 | 5.1E−04 | 9.9E−09 |
| Ab10 | 1.3E+05 | 2.4E−04 | 2.0E−09 | 6.2E+04 | 4.9E−04 | 7.8E−09 |
| Ab11 | 6.5E+04 | 1.2E−03 | 1.8E−08 | 2.4E+04 | 1.7E−03 | 7.2E−08 |
| Ab12 | 9.3E+04 | 2.1E−04 | 2.3E−09 | 3.8E+04 | 4.2E−04 | 1.1E−08 |
| Ab13 | 2.9E+04 | 5.5E−05 | 1.9E−09 | 1.9E+04 | 8.8E−04 | 4.7E−08 |
| Ab14 | 1.9E+05 | 2.1E−04 | 1.1E−09 | 9.1E+04 | 4.5E−04 | 5.0E−09 |
| Ab15 | 7.7E+04 | 5.3E−05 | 6.8E−10 | 2.9E+04 | 9.6E−05 | 3.3E−09 |

hu = human;
cy = cynomolgus monkey;
ECD = extracellular domain;
*= k_{d} manually set to 5E−06 s⁻¹ which was the lower limit of detection for the assay;
E + Y = ×10^{Y};
E − Y = ×10^{-Y}

### Example 5. In vitro Potency of Bcl-xL Inhibitor Antibody-Drug Conjugates (ADCs) Derived from Anti-CD98 Murine Hybridoma Monoclonal Antibodies

### Conjugation of Bcl-xL Inhibitory ADCs

Exemplary ADCs were synthesized using one of nine exemplary methods, described below.

### Materials and Methods

Method A. A solution of BOND-BREAKER tris(2-carboxyethyl)phosphine (TCEP) solution (10 mM, 0.017 mL) was added to a solution of antibody (10 mg/mL, 1 mL) preheated to 37° C. The reaction mixture was kept at 37° C. for 1 hour. The solution of reduced antibody was added to a solution of synthon (3.3 mM, 0.160 mL in DMSO) and gently mixed for 30 minutes. The reaction solution was loaded onto a desalting column (PD10, washed with DPBS 3× before use), followed by DPBS (1.6 mL) and eluted with additional DPBS (3 mL). The purified ADC solution was filtered through a 0.2 micron, low protein-binding 13 mm syringe-filter and stored at 4° C.

Method B. A solution of BOND-BREAKER tris(2-carboxyethyl)phosphine (TCEP) solution (10 mM, 0.017 mL) was added to the solution of antibody (10 mg/mL, 1 mL) preheated to 37° C. The reaction mixture was kept at 37° C. for 1 hour. The solution of reduced antibody was adjusted to pH=8 by adding boric buffer (0.05 mL, 0.5 M, pH8), added to a solution of synthon (3.3 mM, 0.160 mL in DMSO) and gently mixed for 4 hours. The reaction solution was loaded onto a desalting column (PD10, washed with DPBS 3× before use), followed by DPBS (1.6 mL) and eluted with additional DPBS (3 mL). The purified ADC solution was filtered through a 0.2 micron, low protein-binding 13 mm syringe-filter and stored at 4° C.

Method C. Conjugations were performed using a PerkinElmer Janus (part AJL8M01) robotic liquid handling system equipped with an 1235/96 tip ModuLar Dispense Technology (MDT), disposable head (part 70243540) containing a gripper arm (part 7400358), and an 8-tip Varispan pipetting arm (part 7002357) on an expanded deck. The PerkinElmer Janus system was controlled using the WinPREP version 4.8.3.315 Software.

A Pall Filter plate 5052 was pre-wet with 100 μL 1×DPBS using the MDT. Vacuum was applied to the filter plate for 10 seconds and was followed by a 5 second vent to remove DPBS from filter plate. A 50% slurry of Protein A resin (GE MabSelect Sure) in DPBS was poured into an 8 well reservoir equipped with a magnetic ball, and the resin was mixed by passing a traveling magnet underneath the reservoir plate. The 8 tip Varispan arm, equipped with 1 mL conductive tips, was used to aspirate the resin (250 μL) and transfer to a 96-well filter plate. A vacuum was applied for 2 cycles to remove most of the buffer. Using the MDT, 150 μL of 1×PBS was aspirated and dispensed to the 96-well filter plate holding the resin. A vacuum was applied, removing the buffer from the resin. The rinse/vacuum cycle was repeated 3 times. A 2 mL, 96-well collection plate was mounted on the Janus deck, and the MDT transferred 450 μL of 5×DPBS to the collection plate for later use. Reduced antibody (2 mg) as a solution in (200 μL) DPBS was prepared as described above for Conditions A and preloaded into a 96 well plate. The solutions of reduced antibody were transferred to the filter plate wells containing the resin, and the mixture was mixed with the MDT by repeated aspiration/dispensation of a 100 μL volume within the well for 45 seconds per cycle. The aspiration/dispensation cycle was repeated for a total of 5 times over the course of 5 minutes. A vacuum was applied to the filter plate for 2 cycles, thereby removing excess antibody. The MDT tips were rinsed with water for 5 cycles (200 μL, 1 mL total volume). The MDT aspirated and dispensed 150 μL of DPBS to the filter plate wells containing resin-bound antibody, and a vacuum was applied for two cycles. The wash and vacuum sequence was repeated two more times. After the last vacuum cycle, 100 μL of 1×DPBS was dispensed to the wells containing the resin-bound antibody. The MDT then collected 30 μL each of 3.3 mM dimethyl sulfoxide solutions of synthons plated in a 96-well format and dispensed it to the filter plate containing resin-bound antibody in DPBS. The wells containing the conjugation mixture were mixed with the MDT by repeated aspiration/dispensation of a 100 μL volume within the well for 45 seconds per cycle. The aspiration/dispensation sequence was repeated for a total of 5 times over the course of 5 minutes. A vacuum was applied for 2 cycles to remove excess synthon to waste. The MDT tips were rinsed with water for 5 cycles (200 μL, 1 mL total volume). The MDT aspirated and dispensed DPBS (150 μL) to the conjugation mixture, and a vacuum was applied for two cycles. The wash and vacuum sequence was repeated two more times. The MDT gripper then moved the filter plate and collar to a holding station. The MDT placed the 2 mL collection plate containing 450 μL of 10×DPBS inside the vacuum manifold. The MDT reassembled the vacuum manifold by placement of the filter plate and collar. The MDT tips were rinsed with water for 5 cycles (200 μL, 1 mL total volume). The MDT aspirated and dispensed 100 μL of IgG Elution Buffer 3.75 (Pierce) to the conjugation mixture. After one minute, a vacuum was applied for 2 cycles, and the eluent was captured in the receiving plate containing 450 μL of 5×DPBS. The aspiration/dispensation sequence was repeated 3 additional times to deliver ADCsamples with concentrations in the range of 1.5-2.5 mg/mL at pH 7.4 in DPBS.

Method D. Conjugations were performed using a PerkinElmer Janus (part AJL8M01) robotic liquid handling system equipped with an 1235/96 tip ModuLar Dispense Technology (MDT), disposable head (part 70243540) containing a gripper arm (part 7400358), and an 8-tip Varispan pipetting arm (part 7002357) on an expanded deck. The PerkinElmer Janus system was controlled using the WinPREP version 4.8.3.315 Software.

A Pall Filter plate 5052 was prewet with 100 μL 1×DPBS using the MDT. Vacuum was applied to the filter plate for 10 seconds and was followed by a 5 second vent to remove DPBS from filter plate. A 50% slurry of Protein A resin (GE MabSelect Sure) in DPBS was poured into an 8-well reservoir equipped with a magnetic ball, and the resin was mixed by passing a traveling magnet underneath the reservoir plate. The 8 tip Varispan arm, equipped with 1 mL conductive tips, was used to aspirate the resin (250 μL) and transfer to a 96-well filter plate. A vacuum was applied to the filter plate for 2 cycles to remove most of the buffer. The MDT aspirated and dispensed 150 μl of DPBS to the filter plate wells containing the resin. The wash and vacuum sequence was repeated two more times. A 2 mL, 96-well collection plate was mounted on the Janus deck, and the MDT transferred 450 μL of 5×DPBS to the collection plate for later use. Reduced antibody (2 mg) as a solution in (200 μL) DPBS was prepared as described above for Conditions A and dispensed into the 96-well plate. The MDT then collected 30 μL each of 3.3 mM dimethyl sulfoxidesolutions of synthons plated in a 96-well format and dispensed it to the plate loaded with reduced antibody in DPBS. The mixture was mixed with the MDT by twice repeated aspiration/dispensation of a 100 μL volume within the well. After five minutes, the conjugation reaction mixture (230 ML) was transferred to the 96-well filter plate containing the resin. The wells containing the conjugation mixture and resin were mixed with the MDT by repeated aspiration/dispensation of a 100 μL volume within the well for 45 seconds per cycle. The aspiration/dispensation sequence was repeated for a total of 5 times over the course of 5 minutes. A vacuum was applied for 2 cycles to remove excess synthon and protein to waste. The MDT tips were rinsed with water for 5 cycles (200 μL, 1 mL total volume). The MDT aspirated and dispensed DPBS (150 μL) to the conjugation mixture, and a vacuum was applied for two cycles. The wash and vacuum sequence was repeated two more times. The MDT gripper then moved the filter plate and collar to a holding station. The MDT placed the 2 mL collection plate containing 450 μL of 10×DPBS inside the vacuum manifold. The MDT reassembled the vacuum manifold by placement of the filter plate and collar. The MDT tips were rinsed with water for 5 cycles (200 μL, 1 mL total volume). The MDT aspirated and dispensed 100 μL of IgG Elution Buffer 3.75 (P) to the conjugation mixture. After one minute, a vacuum was applied for 2 cycles, and the eluent was captured in the receiving plate containing 450 μL of 5×DPBS. The aspiration/dispensation sequence was repeated 3 additional times to deliver ADC samples with concentrations in the range of 1.5-2.5 mg/mL at pH 7.4 in DPBS.

Method E. A solution of BOND-BREAKER tris(2-carboxyethyl)phosphine (TCEP) solution (10 mM, 0.017 mL) was added to the solution of antibody (10 mg/mL, 1 mL) at room temperature. The reaction mixture was heated to 37° C. for 75 minutes. The solution of reduced antibody cooled to room temperature and was added to a solution of synthon (10 mM, 0.040 mL in DMSO) followed by addition of boric buffer (0.1 mL, 1M, pH 8). The reaction solution was let to stand for 3 days at room temperature, loaded onto a desalting column (PD10, washed with DPBS 3×5 mL before use), followed by DPBS (1.6 mL) and eluted with additional DPBS (3 mL). The purified ADC solution was filtered through a 0.2 micron, low protein-binding 13 mm syringe-filter and stored at 4° C.

Method F. Conjugations were performed using a Tecan Freedom Evo robotic liquid handling system. The solution of antibody (10 mg/mL) was preheated to 37° C., and aliquoted to a heated 96 deep-well plate in amounts of 3 mg per well (0.3 mL) and kept at 37° C. A solution of BOND-BREAKER tris(2-carboxyethyl)phosphine (TCEP) solution (1 mM, 0.051 mL/well) was added to antibodies, and the reaction mixture was kept at 37° C. for 75 minutes. The solution of reduced antibody was transferred to an unheated 96 deep-well plate. Corresponding solutions of synthons (5 mM, 0.024 mL in DMSO) were added to the wells with reduced antibodies and treated for 15 minutes. The reaction solutions were loaded onto a platform (8×12) of desalting columns (NAPS, washed with DPBS 4× before use), followed by DPBS (0.3 mL) and eluted with additional DPBS (0.8 mL). The purified ADC solutions were further aliquoted for analytics and stored at 4° C.

Method G. Conjugations were performed using a Tecan Freedom Evo robotic liquid handling system. The solution of antibody (10 mg/mL) was preheated to 37° C., and aliquoted onto a heated 96 deep-well plate in amounts of 3 mg per well (0.3 mL) and kept at 37 C. A solution of BOND-BREAKER tris(2-carboxyethyl)phosphine (TCEP) solution (1 mM, 0.051 mL/well) was added to antibodies, and the reaction mixture was kept at 37° C. for 75 minutes. The solutions of reduced antibody were transferred to an unheated 96 deep-well plate. Corresponding solutions of synthons (5 mM, 0.024 mL/well in DMSO) were added to the wells with reduced antibodies followed by addition of boric buffer (pH=8, 0.03 mL/well) and treated for 3 days. The reaction solutions were loaded onto a platform (8×12) of desalting columns (NAPS, washed with DPBS 4× before use), followed by DPBS (0.3 mL) and eluted with additional DPBS (0.8 mL). The purified ADC solutions were further aliquoted for analytics and stored at 4° C.

Method H. A solution of BOND-BREAKER tris(2-carboxyethyl)phosphine (TCEP) solution (10 mM, 0.17 mL) was added to the solution of antibody (10 mg/mL, 10 mL) at room temperature. The reaction mixture was heated to 37° C. for 75 minutes. The solution of synthon (10 mM, 0.40 mL in DMSO) was added to a solution of reduced antibody cooled to room temperature. The reaction solution was let to stand for 30 minutes at room temperature. The solution of ADC was treated with saturated ammonium sulfate solution (˜2-2.5 mL) until a slightly cloudy solution formed. This solution was loaded onto butyl sepharose column (5 mL of butyl sepharose) equilibrated with 30% phase B in phase A (phase A: 1.5 M ammonium sulfate, 25 mM phosphate: phase B: 25 mM phosphate, 25% isopropanol v/v). Individual fractions with DAR2 (also referred to as “E2”) and DAR4 (also referred to as “E4”) eluted upon applying gradient A/B up to 75% phase B. Each ADC solution was concentrated and buffer switched using centrifuge concentrators or TFF for larger scales. The purified ADC solutions were filtered through a 0.2 micron, low protein-binding 13 mm syringe-filter and stored at 4° C.

Method I. A solution of BOND-BREAKER tris(2-carboxyethyl)phosphine (TCEP) solution (10 mM, 0.17 mL) was added to the solution of antibody (10 mg/mL, 10 mL) at room temperature. The reaction mixture was heated to 37° C. for 75 minutes. The solution of synthon (10 mM, 0.40 mL in DMSO) was added to a solution of reduced antibody cooled to room temperature. The reaction solution was let to stand for 30 minutes at room temperature. The solution of ADC was treated with saturated ammonium sulfate solution (˜2-2.5 mL) until a slightly cloudy solution formed. This solution was loaded onto a butyl sepharose column (5 mL of butyl sepharose) equilibrated with 30% phase B in Phase A (phase A: 1.5 M ammonium sulfate, 25 mM phosphate: phase B: 25 mM phosphate, 25% isopropanol v/v). Individual fractions with DAR2 (also referred to as “E2”) and DAR 4 (also referred to as “E4”) eluted upon applying a gradient A/B up to 75% phase B. Each ADC solution was concentrated and buffer switched using centrifuge concentrators or TFF for larger scales.

The ADC solutions were treated with boric buffer (0.1 mL, 1M, pH8). The reaction solution was let stand for 3 days at room temperature, then loaded onto a desalting column (PD10, washed with DPBS 3×5 mL before use), followed by DPBS (1.6 mL) and eluted with additional DPBS (3 mL). The purified ADC solution was filtered through a 0.2 micron, low protein-binding 13 mm syringe-filter and stored at 4° C.

The DAR and percentage aggregation of exemplary ADCs synthesized as described above, were determined by LC-MS and size exclusion chromatography (SEC), respectively.

LC-MS General Methodology

LC-MS analysis was performed using an Agilent 1100 HPLC system interfaced to an Agilent LC/MSD TOF 6220 ESI mass spectrometer. The ADC was reduced with 5 mM (final concentration) BOND-BREAKER TCEP solution (Thermo Scientific, Rockford, Ill.), loaded onto a Protein Microtrap (Michrom Bioresorces, Auburn, Calif.) desalting cartridge, and eluted with a gradient of 10% B to 75% B in 0.2 minutes at ambient temperature. Mobile phase A was H₂0 with 0.1% formic acid (FA), mobile phase B was acetonitrile with 0.1% FA, and the flow rate was 0.2 ml/min. Electrospray-ionization time-of-flight mass spectra of the co-eluting light and heavy chains were acquired using Agilent MassHunter™ acquisition software. The extracted intensity vs, m/z spectrum was deconvoluted using the Maximum Entropy feature of MassHunter software to determine the mass of each reduced antibody fragment. DAR was calculated from the deconvoluted spectrum by summing intensities of the naked and modified peaks for the light chain and heavy chain, normalized by multiplying intensity by the number of drugs attached. The summed, normalized intensities were divided by the sum of the intensities, and the summing results for two light chains and two heavy chains produced a final average DAR value for the full ADC.

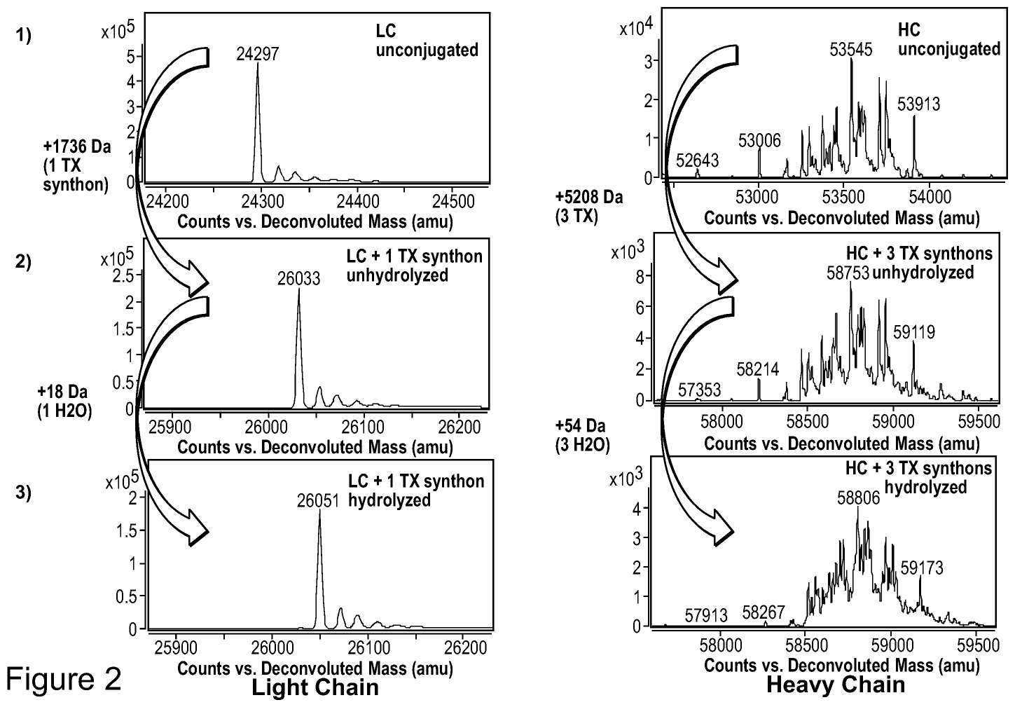

Thiosuccinimide hydrolysis of a bioconjugate can be monitored by electrospray mass spectrometry, since the addition of water to the conjugate results in an increase of 18 Daltons to the observable molecular weight of the conjugate. When a conjugate is prepared by fully reducing the interchain disulfides of a human IgG1 antibody and conjugating the maleimide derivative to each of the resulting cysteines, each light chain of the antibody will contain a single maleimide modification and each heavy chain will contain three maleimide modifications (see FIG. 1). Upon complete hydrolysis of the resulting thiosuccinimides, the mass of the light chain will therefore increase by 18 Daltons, while the mass of each heavy chain will increase by 54 Daltons. This is illustrated in FIG. 2, with the conjugation and subsequent hydrolysis of an exemplary maleimide drug-linker (synthon TX, molecular weight X1736 Da) to the fully reduced antibody huAb108. The presence of multiple glycosylation sites on the heavy chain results in the heterogeneity of mass observed.

### Size Exclusion Chromatography General Methodology

Size exclusion chromatography was performed using a Shodex KW802.5 column in 0.2M potassium phosphate pH 6.2 with 0.25 mM potassium chloride and 15% IPA at a flow rate of 0.75 ml/min. The peak area absorbance at 280 nm was determined for each of the high molecular weight and monomeric eluents by integration of the area under the curve. The % aggregate fraction of the conjugate sample was determined by dividing the peak area absorbance at 280 nM for the high molecular weight eluent by the sum of the peak area absorbances at 280 nM of the high molecular weight and monomeric eluents multiplied by 100%.

### Conjugation of Murine Anti-CD98 Antibodies

The above fifteen purified murine anti-CD98 mAbs were first conjugated with the Bcl-xL inhibitor payload CZ according to Method A, as set forth above. The activity of these ADCs were tested in growth inhibition assays in three human cancer cell lines expressing endogenous CD98: HCC38 breast cancer cell line, Molt-4 human acute lymphoblastic leukemia cell line, and Jurkat acute T cell leukemia cell line. Briefly, 3000 cells per well were plated into 96-well plates, and were treated with ADCs in serial dilutions for either 2-days (Molt-4 cells), 4-days (HCC38 cells), or 5-days (Jurkat cells). The number of viable cells was determined by the CellTiter-Glor® reagent (Promega G7572) as instructed by the manufacturer. Data was analyzed using Graphpad Prism software and IC₅₀ values were reported as the concentration of ADC to achieve 50% inhibition of cell proliferation (Table 5).

TABLE 5
In vitro Potency of Bcl-xL Inhibitor ADCs Conjugated
to Anti-CD98 Murine Hybridoma Antibodies.

| | DAR | % | | | |
|---|---|---|---|---|---|
| | by | Aggregates | ADC potency, IC₅₀ (nM) | | |
| ADC | MS | by SEC | HCC38 | Molt-4 | Jurkat |
| Ab1-CZ | 2.3 | 6.9 | 0.998 | 0.053 | 0.050 |
| Ab2-CZ | 2.9 | 7.6 | 0.791 | 0.050 | 0.065 |
| Ab3-CZ | 2.2 | 7.2 | 0.561 | 0.031 | 0.071 |
| Ab4-CZ | 1.6 | 5.1 | 0.879 | 0.127 | 0.371 |
| Ab5-CZ | 2.6 | 8.7 | 0.728 | 0.022 | 0.137 |
| Ab6-CZ | 2.0 | 11.2 | 0.438 | 0.057 | 0.086 |
| Ab7-CZ | 1.4 | 4.7 | 1.149 | 0.079 | 0.134 |
| Ab8-CZ | 2.3 | 4.1 | 0.963 | 0.079 | 0.121 |
| Ab9-CZ | 2.7 | 4.2 | 1.091 | 0.030 | 0.092 |
| Ab10-CZ | 2.8 | 4.7 | 1.158 | 0.047 | 0.148 |
| Ab11-CZ | 3.6 | 4.2 | 1.139 | 0.279 | 0.311 |
| Ab12-CZ | 3.0 | 2.9 | 0.685 | 0.087 | 0.094 |
| Ab13-CZ | 2.3 | 4.7 | 0.787 | 0.380 | 0.149 |
| Ab14-CZ | 2.0 | 8.8 | 0.798 | 0.025 | 0.063 |
| Ab15-CZ | 2.3 | 5.7 | 1.056 | 0.071 | 0.076 |
| *MSL109-CZ | 3.3 | 3 | >100 | >100 | >100 |

DAR = drugs/antibody ratio;
MS = Mass spectrometry;
SEC = Size exclusion chromatography;

*MSLIO9 is a humanized IgG1 antibody that binds to cytomegalovirus (CMV) glycoprotein H. It is used as a negative control mAb.

### Example 6. In Vivo Potency of Bcl-xL Inhibitor Antibody-Drug Conjugates (ADCs) Derived from Anti-CD98 Murine Hybridoma Monoclonal Antibodies

The in vivo efficacy of anti-CD98 hybridoma mAb conjugates were tested using Ab3-CZ and Ab5-CZ as examples in NCI—H146 (human small cell lung cancer) xenograft model. The two anti-CD98 hybridoma mAb, Ab3-CZ and Ab5-CZ, were conjugated to the Bcl-xL inhibitor synthon CZ according to Method A. NCI—H146 was obtained from the American Type Culture Collection (ATCC, Manassas, Va.). The cells were cultured as monolayers in RPMI-1640 culture media (Invitrogen, Carlsbad, Calif.) that was supplemented with 10% Fetal Bovine Serum (FBS, Hyclone, Logan, Utah). To generate xenografts, 5×10⁶ (NCI—H146) viable cells were inoculated subcutaneously into the right flank of immune deficient female SCID-bg mice (Charles River Laboratories, Wilmington, Mass.). The injection volume was 0.2 mL and composed of Matrigel (BD, Franklin Lakes, N.J.). Tumors were size matched at approximately 212 mm³. Antibodies and conjugates were formulated in phosphate buffered saline, pH 7.2 and injected intraperitoneally. Injection volume did not exceed 200 μL. Therapy began within 24 hours after size matching of the tumors. Mice weighed approximately 21 g at the onset of therapy. Tumor volume was estimated two to three times weekly. Measurements of the length (L) and width (W) of the tumor were taken via electronic caliper and the volume was calculated according to the following equation: V=L×W²/2. Mice were euthanized when tumor volume reached 3,000 mm³ or skin ulcerations occurred. Eight mice were housed per cage. Food and water were available ad libittun. Mice were acclimated to the animal facilities for a period of at least one week prior to commencement of experiments. Animals were tested in the light phase of a 12-hour light: 12-hour dark schedule (lights on at 06:00 hours). Anti-CD98 conjugates (10 mg/kg) were administered as a single dose (QDx1) intraperitoneally. A human IgG control antibody (MSL109, a humanized IgG1 antibody that binds to cytomegalovirus (CMV) glycoprotein H) was used as a negative control agent.

To refer to efficacy of therapeutic agents, parameters of amplitude (TGIₘₐₓ), durability (TGD) and response frequency (CR, PR, OR) of therapeutic response are used. The efficacy of inhibition of NCI—H146 xenografts growth with CD98-targeted ADCs is illustrated by Table 6, below. In the tables, to refer to efficacy, parameters of amplitude (TGIₘₐₓ) and durability (TGD) of therapeutic response are used. TGIₘₐₓ is the maximum tumor growth inhibition during the experiment. Tumor growth inhibition is calculated by 100*(1−Tᵥ/Cᵥ) where Tᵥ and Cᵥ are the mean tumor volumes of the treated and control groups, respectively. TGD or tumor growth delay is the extended time of a treated tumor needed to reach a volume of 1 cm³ relative to the control group. TGD is calculated by 100*(Tₜ/Cₜ−1) where Tₜ and Cₜ are the median time periods to reach 1 cm³ of the treated and control groups, respectively. Distribution of the response amplitude in a specific group is given by the frequency of complete responders (CR), partial responders (PR), and overall responders (OR). CR is the percentage of mice within a group with a tumor burden of 25 mm³ for at least three measurements. PR is the percentage of mice within a group with a tumor burden larger than 25 mm³ but less than one-half of the volume at onset of treatment for at least three measurements. OR is the sum of CR and PR.

TABLE 6
Inhibition of NCI-H146 Xenograft Tumor Growth after
Treatment with a Single Dose of CD98-targeting Bcl-xLi ADC

| | | Dose^{[a]}/ | Growth Inhibition | | Response Frequency | | |
|---|---|---|---|---|---|---|---|
| | *ADC | route/ | TGIₘₐₓ | TGD | CR | PR | OR |
| Treatment | DAR | regimen | (%) | (%) | (%) | (%) | (%) |
| MSL109** | | 10/IP/QDx1 | 0 | 0 | 0 | 0 | 0 |
| MSL109-CZ† | 4.2 | 10/IP/QDxl | 34 | 10 | 0 | 0 | 0 |
| Ab3-CZ | 3.3 | 10/IP/QDx1 | 79* | 81* | 0 | 50 | 50 |
| Ab5-CZ | 3.2 | 10/IP/QDx1 | 76* | 76* | 0 | 0 | 0 |

*DAR = drugs/antibody ratio as determined by mass spectrometry;
**Negative control IgG1 mAb that binds to cytomegalovirus (CMV) glycoprotein H;
†Non-targeting antibody drug conjugate;
^{[a]}dose is given in mg/kg/day;
*= p < 0.05 as compared to control treatment.

### Example 7. Generation of Recombinant Anti-CD98 Chimeric Antibodies

The heavy and light chain variable regions (VH and VL) corresponding to the anti-CD98 murine hybridoma antibodies were rescued from hybridoma cells by reverse transcriptase-polymerase chain reaction (RT-PCR). The identified variable regions were expressed in mammalian host cells, as chimeric antibodies, in the context of a human IgG1(L234A, L235A) heavy chain and kappa light chain constant regions, respectively. Table 7 lists these anti-CD98 chimera mAbs generated and their corresponding hybridoma origin. The variable region sequences of these chimera mAbs are summarized in Tables8 and 9.

TABLE 7
A List of Recombinant anti-CD98 Chimera Antibodies

| Chimera | Source Hybridoma |
|---|---|
| mAb | mAb |
| ChAb1 | Ab1 |
| ChAb2 | Ab2 |
| ChAb3 | Ab3 |
| ChAb4 | Ab4 |
| ChAb5 | Ab5 |
| ChAb6 | Ab6 |
| ChAb7 | Ab7 |
| ChAb8 | Ab8 |
| ChAb9 | Ab9 |
| ChAb10 | Ab10 |
| ChAb11 | Ab11 |
| ChAb12 | Ab12 |
| ChAb13 | Ab13 |
| ChAb14 | Ab14 |
| ChAb15 | Ab15 |

TABLE 8
Variable Region Sequences of Chimeric Anti-CD98 Antibodies from
Mouse Hybridomas

| | | Protein | | |
|---|---|---|---|---|
| SEQ ID NO: | Clone | Region | Residues | V Region |
| 1 | chAb1 | VH | | EVKLVESGGGLVQPGGSLRLS |
| | | | | CATSGFTFTDYYMSWVRQPPG |
| | | | | KALEWLGFIRNPANVYTTEYS |
| | | | | ASVKGRFTISRDNSQSILYLQ |
| | | | | MNTLRAEDSATYYCARASYGN |
| | | | | SEGWFAYWGQGTLVTVSA |
| 2 | chAb1 | CDR-H1 | Residues 26-35 | GFTFTDYYMS |
| | | | of SEQ ID NO.: 1 | |
| 3 | chAb1 | CDR-H2 | Residues 50-68 | FIRNPANVYTTEYSASVKG |
| | | | of SEQ ID NO.: 1 | |
| 4 | chAb1 | CDR-H3 | Residues 101-112 | ASYGNSEGWFAY |
| | | | of SEQ ID NO.: 1 | |
| 5 | chAb1 | VL | | DIVMSQSPSSLAVSVGEKVTM |
| | | | | SCKSSQNLLYNNNQKNYLAWY |
| | | | | QQKPGQSPKLLIYWASTRESG |
| | | | | VPDRFTGSGSGTDFTLTISSV |
| | | | | KAEDLAVYYCQQYYSYPRTFG |
| | | | | GGTKLEIK |
| 6 | chAb1 | CDR-L1 | Residues 24-40 | KSSQNLLYNNNQKNYLA |
| | | | of SEQ ID NO.: 5 | |
| 7 | chAb1 | CDR-L2 | Residues 56-62 | WASTRES |
| | | | of SEQ ID NO.: 5 | |
| 8 | chAb1 | CDR-L3 | Residues 95-103 | QQYYSYPRT |
| | | | of SEQ ID NO.: 5 | |
| 9 | chAb2 | VH | | EVKLVESGGGLVQPGGSLRLS |
| | | | | CATSGFNFTDYYMSWVRQPPG |
| | | | | KALEWLGFIRNKANGYTTEYS |
| | | | | ASVKGRFTISRDDSQSILYLQ |
| | | | | MNTLRAEDSATYYCARASYGN |
| | | | | SEGWFAYWGQGTLVTVSA |
| 10 | chAb2 | CDR-H1 | Residues 26-35 | GFNFTDYYMS |
| | | | of SEQ ID NO.: 9 | |
| 11 | chAb2 | CDR-H2 | Residues 50-68 | FIRNKANGYTTEYSASVKG |
| | | | of SEQ ID NO.: 9 | |
| 4 | chAb2 | CDR-H3 | Residues 101-112 | ASYGNSEGWFAY |
| | | | of SEQ ID NO.: 9 | |
| 12 | chAb2 | VL | | DIVMSQSPSSLAVSVGEKVTM |
| | | | | NCKSSQSLLYSSNQKNYLAWY |
| | | | | QQKPGQSPKLLIYWASTRESG |
| | | | | VPDRFTGSGSGTDFTLTISSV |
| | | | | KAEDLAVYYCQQYYRYPRTFG |
| | | | | GGTKLEIK |
| 13 | chAb2 | CDR-L1 | Residues 24-40 | KSSQSLLYSSNQKNYLA |
| | | | of SEQ ID NO.: 12 | |
| 7 | chAb2 | CDR-L2 | Residues 56-62 | WASTRES |
| | | | of SEQ ID NO.: 12 | |
| 14 | chAb2 | CDR-L3 | Residues 95-103 | QQYYRYPRT |
| | | | of SEQ ID NO.: 12 | |
| 15 | chAb3 | VH | | EVKLVESGGGLVQPGNSLRLS |
| | | | | CATSGFTFIDYYMSKVRQSPG |
| | | | | KALEWLGFIRNKANGYTTEYS |
| | | | | ASVKGRFTISRDNSQSILYLQ |
| | | | | MDTLRAEDSATYYCTRDRPAW |
| | | | | FVYWGQGTLVTVSA |
| 16 | chAb3 | CDR-H1 | Residues 26-35 | GFTFIDYYMS |
| | | | of SEQ ID NO.: 15 | |
| 11 | chAb3 | CDR-H2 | Residues 50-68 | FIRNKANGYTTEYSASVKG |
| | | | of SEQ ID NO.: 15 | |
| 17 | chAb3 | CDR-H3 | Residues 101-108 | DRPAWFVY |
| | | | of SEQ ID NO.: 15 | |
| 18 | chAb3 | VL | | DIVMSQSPSSLAVSVGEKVTM |
| | | | | SCKSSQSLLYSSNQKNYLAWY |
| | | | | QQKPGQSPKLLIYWASTRESG |
| | | | | VPDRFTGSGSGTDFTLTFSSV |
| | | | | RAEDLAVYYCQQYYSYPYTFG |
| | | | | GGTKLEIK |
| 13 | chAb3 | CDR-L1 | Residues 24-40 | KSSQSLLYSSNQKNYLA |
| | | | of SEQ ID NO.: 18 | |
| 7 | chAb3 | CDR-L2 | Residues 56-62 | WASTRES |
| | | | of SEQ ID NO.: 18 | |
| 19 | chAb3 | CDR-L3 | Residues 95-103 | QQYYSYPYT |
| | | | of SEQ ID NO.: 18 | |
| 20 | chAb4 | VH | | EVKLVESGGGLVQPGGSLRLS |
| | | | | CTTSGFTFTDYYMSWVRQPPG |
| | | | | KALEWLGFIRNKATIYTTEYS |
| | | | | ASVKGRFTISRDNSQSILYLQ |
| | | | | MNTLRAEDSATYYCARASYGN |
| | | | | SEGWFAYWGQGTLVTVSA |
| 2 | chAb4 | CDR-H1 | Residues 26-35 | GFTFTDYYMS |
| | | | of SEQ ID NO.: 20 | |
| 21 | chAb4 | CDR-H2 | Residues 50-68 | FIRNKATIYTTEYSASVKG |
| | | | of SEQ ID NO.: 20 | |
| 4 | chAb4 | CDR-H3 | Residues 101-112 | ASYGNSEGWFAY |
| | | | of SEQ ID NO.: 20 | |
| 22 | chAb4 | VL | | DIVMSQSPSSLAVSVGEKVTM |
| | | | | SCKSSQSLLYSSNQKNYLAWY |
| | | | | QQKPGQSPKVLIYWASTRESG |
| | | | | VPDRFTGSGSGTDFTLTISSV |
| | | | | KAEDLAVYYCQQYYSYPRTFG |
| | | | | GGTKLEIK |
| 13 | chAb4 | CDR-L1 | Residues 24-40 | KSSQSLLYSSNQKNYLA |
| | | | of SEQ ID NO.: 22 | |
| 7 | chAb4 | CDR-L2 | Residues 56-62 | WASTRES |
| | | | of SEQ ID NO.: 22 | |
| 8 | chAb4 | CDR-L3 | Residues 95-103 | QQYYSYPRT |
| | | | of SEQ ID NO.: 22 | |
| 23 | chAb5 | VH | | EVKLVESGGGLVQPGGSLRLS |
| | | | | CATSGFTFTDYYMTKVRQPPG |
| | | | | KALEWLGFIRNKANGYTTEYS |
| | | | | ASVKGRFTISRDNSLSILYLQ |
| | | | | MNTLRAEDSATYYCARASYVN |
| | | | | SEGWFAYWGQGTLVTVSA |
| 24 | chAb5 | CDR-H1 | Residues 26-35 | GFTFTDYYMT |
| | | | of SEQ ID NO.: 23 | |
| 11 | chAb5 | CDR-H2 | Residues 50-68 | FIRNKANGYTTEYSASVKG |
| | | | of SEQ ID NO.: 23 | |
| 25 | chAb5 | CDR-H3 | Residues 101-112 | ASYVNSEGWFAY |
| | | | of SEQ ID NO.: 23 | |
| 26 | chAb5 | VL | | DIVMSQSPSSLAVSVGEKVTM |
| | | | | SCKSSQSLLYSSNQKNYLAWY |
| | | | | QQKLGQSPKLLIYWASTRESG |
| | | | | VPDRFTGSGSGTDFTLTISSV |
| | | | | KAEDLAVYYCQHYYSYPRTFG |
| | | | | GGTKLEIK |
| 13 | chAb5 | CDR-L1 | Residues 24-40 | KSSQSLLYSSNQKNYLA |
| | | | of SEQ ID NO.: 26 | |
| 7 | chAb5 | CDR-L2 | Residues 56-62 | WASTRES |
| | | | of SEQ ID NO.: 26 | |
| 27 | chAb5 | CDR-L3 | Residues 95-103 | QHYYSYPRT |
| | | | of SEQ ID NO.: 26 | |

TABLE 9
Variable Region Sequences of Anti-CD98 Antibodies from Rat Hybridomas

| SEQ ID | | Protein | | |
|---|---|---|---|---|
| NO: | Clone | Region | Residues | V Region |
| 28 | chAb6 | VH | | QVQLKESGPGLAQPSQTLSL |
| | | | | TCTVSGFSLSTYGVIWLRQP |
| | | | | PGKGLEWMGVIWTNGNTNYN |
| | | | | STLKSRLSISRDTSESQVYL |
| | | | | QMNSLQTEDTATYYCARHYY |
| | | | | DGAYYYGYFDYWGQGVMVTV |
| | | | | SS |
| 29 | chAb6 | CDR-H1 | Residues 26-35 | GFSLSTYGVI |
| | | | of SEQ ID NO.: 28 | |
| 30 | chAb6 | CDR-H2 | Residues 50-65 | VIWTNGNTNYNSTLKS |
| | | | of SEQ ID NO.: 28 | |
| 31 | chAb6 | CDR-H3 | Residues 98-3111 | HYYDGAYYYGYFDY |
| | | | of SEQ ID NO.: 28 | |
| 32 | chAb6 | | | DIVMTQTPSSQAVSAGEKVT |
| | | | | MSCKSSQSLLYSENKKNYLA |
| | | | | WYQQKPGQSPKLLIYWASTR |
| | | | | ESGVPDRFIGSGSGTDFTLT |
| | | | | ISSVQAEDLAVYYCQQYYYF |
| | | | | PYTFGAGTKLELK |
| 33 | chAb6 | CDR-L1 | Residues 24-40 | KSSQSLLYSENKKNYLA |
| | | | of SEQ ID NO.: 32 | |
| 7 | chAb6 | CDR-L2 | Residues 56-62 | WASTRES |
| | | | of SEQ ID NO.: 32 | |
| 34 | chAb6 | CDR-L3 | Residues 95-103 | QQYYYFPYT |
| | | | of SEQ ID NO.: 32 | |
| 35 | chAb7 | VH | | QVQLKESGPGLVQPSQTLSL |
| | | | | TCTVSGFSLSTYGVIWVRQP |
| | | | | PGKGLEKMGVIWANGNTNYN |
| | | | | STLKSRLSISRDTSKSQVYL |
| | | | | KMNSLQTEDTATYYCARHYY |
| | | | | DGTYYYGYFDYWGQGVMVTV |
| | | | | SS |
| 29 | chAb7 | CDR-H1 | Residues 26-35 | GFSLSTYGVI |
| | | | of SEQ ID NO.: 35 | |
| 36 | chAb7 | CDR-H2 | Residues 50-65 | VIWANGNTNYNSTLKS |
| | | | of SEQ ID NO.: 35 | |
| 37 | chAb7 | CDR-H3 | Residues 98-111 | HYYDGTYYYGYFDY |
| | | | of SEQ ID NO.: 35 | |
| 38 | chAb7 | VL | | DIVMTQTPSSQAVSAGEKVT |
| | | | | MNCKSSQSLLYSENKKNYLA |
| | | | | WYQQKPGQSPKLLIYWASTR |
| | | | | ESGVPDRFIGSGSGTDFTLT |
| | | | | ISSVQAEDLAVYYCQQYYYF |
| | | | | PYTFGPGTKLELK |
| 33 | chAb7 | CDR-L1 | Residues 24-40 | KSSQSLLYSENKKNYLA |
| | | | of SEQ ID NO.: 38 | |
| 7 | chAb7 | CDR-L2 | Residues 56-62 | WASTRES |
| | | | of SEQ ID NO.: 38 | |
| 34 | chAb7 | CDR-L3 | Residues 95-103 | QQYYYFPYT |
| | | | of SEQ ID NO.: 38 | |
| 39 | chAb8 | VH | | EVQLVESGGGLVQPGRSLKL |
| | | | | SCAASGFTFSDYAMAWVRQA |
| | | | | PKKGLEWVASIIYDGRGTYY |
| | | | | RDSVKGRFTISRDNAKSTLY |
| | | | | LQMDSLRSEDTATYYCARQG |
| | | | | DGTYYYWGYFDYWGQGVMVT |
| | | | | VSS |
| 40 | chAb8 | CDR-H1 | Residues 26-35 | GFTFSDYAMA |
| | | | of SEQ ID NO.: 39 | |
| 41 | chAb8 | CDR-H2 | Residues 50-66 | SIIYDGRGTYYRDSVKG |
| | | | of SEQ ID NO.: 39 | |
| 42 | chAb8 | CDR-H3 | Residues 99-112 | QGDGTYYYWGYFDY |
| | | | of SEQ ID NO.: 39 | |
| 43 | chAb8 | VL | | DIVMTQSPSSLAVSAGETVT |
| | | | | INCKSSQSLLSSGNQKNYLA |
| | | | | WYQQKPGQSPKLLIYWASTR |
| | | | | QSGVPDRFIGSGSGTDFTLT |
| | | | | INSVQAEDLAIYYCQQYYDT |
| | | | | PYTFGAGTKLELK |
| 44 | chAb8 | CDR-L1 | Residues 24-40 | KSSQSLLSSGNQKNYLA |
| | | | of SEQ ID NO.: 43 | |
| 45 | chAb8 | CDR-L2 | Residues 56-62 | WASTRQS |
| | | | of SEQ ID NO.: 43 | |
| 46 | chAb8 | CDR-L3 | Residues 95-103 | QQYYDTPYT |
| | | | of SEQ ID NO.: 43 | |
| 47 | chAb9 | VH | | QVQLKESGPGLVQPSQTLSL |
| | | | | TCAVSGFSLSNYGVIWVRQP |
| | | | | PGKGLEWMAVIWTNGNTNYN |
| | | | | STLKSRLSISRDTSKSQVYL |
| | | | | KMNSLQTEDTATYYCARHYY |
| | | | | DGTYYYGYFDYWGQGVMVTV |
| | | | | SS |
| 48 | chAb9 | CDR-H1 | Residues 26-35 | GFSLSNYGVI |
| | | | of SEQ ID NO.: 47 | |
| 30 | chAb9 | CDR-H2 | Residues 50-65 | VIWTNGNTNYNSTLKS |
| | | | of SEQ ID NO.: 47 | |
| 37 | chAb9 | CDR-H3 | Residues 98-111 | HYYDGTYYYGYFDY |
| | | | of SEQ ID NO.: 47 | |
| 49 | chAb9 | VL | | DIVMTQTPSSQAVSAGEKVT |
| | | | | MSCKSSQSLLYTENKKNYLA |
| | | | | WYQQKPGQSPKLLIYWASTR |
| | | | | ESGVPDRFMGSGSGTDFTLT |
| | | | | ISSVQAEDLAVYYCQQYYYF |
| | | | | PYMFGAGTKLELK |
| 50 | chAb9 | CDR-L1 | Residues 24-40 | KSSQSLLYTENKKNYLA |
| | | | of SEQ ID NO.: 49 | |
| 7 | chAb9 | CDR-L2 | Residues 56-62 | WASTRES |
| | | | of SEQ ID NO.: 49 | |
| 51 | chAb9 | CDR-L3 | Residues 95-103 | QQYYYFPYM |
| | | | of SEQ ID NO.: 49 | |
| 52 | chAb10 | VH | | EVQLVESGGGLVQPGRSLKL |
| | | | | SCAASGFTFSDYAMAWVRQA |
| | | | | PKKSLEWVATIIYDGRGTYC |
| | | | | RDSVKGRFTISRDNAKSTLY |
| | | | | LQMDSLRSEDTATYYCARQG |
| | | | | DGTYHYWGYFDYWGQGVMVT |
| | | | | VSS |
| 40 | chAb10 | CDR-H1 | Residues 26-35 | GFTFSDYAMA |
| | | | of SEQ ID NO.: 52 | |
| 53 | chAb10 | CDR-H2 | Residues 50-66 | TIIYDGRGTYCRDSVKG |
| | | | of SEQ ID NO.: 52 | |
| 54 | chAb10 | CDR-H3 | Residues 99-112 | QGDGTYHYWGYFDY |
| | | | of SEQ ID NO.: 52 | |
| 55 | chAb10 | VL | | DIVMTQSPSSLAVSAGETVT |
| | | | | INCKSSQSLLSSGNQKNYLA |
| | | | | WYQQKPGQSPKLLIYWASTR |
| | | | | QSGVPDRFIGSGSGTDFTLT |
| | | | | ISSVQAEDLAIYYCQQYYDT |
| | | | | PYTFGAGTKVDLK |
| 44 | chAb10 | CDR-L1 | Residues 24-40 | KSSQSLLSSGNQKNYLA |
| | | | of SEQ ID NO.: 55 | |
| 45 | chAb10 | CDR-L2 | Residues 56-62 | WASTRQS |
| | | | of SEQ ID NO.: 55 | |
| 46 | chAb10 | CDR-L3 | Residues 95-103 | QQYYDTPYT |
| | | | of SEQ ID NO.: 55 | |
| 56 | chAb11 | VH | | EVQLVESGGGLVQPGRSLKL |
| | | | | SCAASGFTFSDYAMAWVRQA |
| | | | | PKKGLEWVAGIIYDGRGTYY |
| | | | | RDSVKGRFTISRDNAKSTLY |
| | | | | LQMDSLRSEDTATYYCARQG |
| | | | | DGTYYYWGYFDYWGQGVMVT |
| | | | | VSS |
| 40 | chAb11 | CDR-H1 | Residues 26-35 | GFTFSDYAMA |
| | | | of SEQ ID NO.: 56 | |
| 57 | chAb11 | CDR-H2 | Residues 50-66 | GIIYDGRGTYYRDSVKG |
| | | | of SEQ ID NO.: 56 | |
| 42 | chAb11 | CDR-H3 | Residues 99-112 | QGDGTYYYWGYFDY |
| | | | of SEQ ID NO.: 56 | |
| 58 | chAb11 | VL | | DIVMTQSPSSLAVSAGETVT |
| | | | | INCRSSQSLLSSGNQKNYLA |
| | | | | WYQQKPGQSPKLLIYWASTR |
| | | | | QSGVPDRFIGSGSGTDFTLT |
| | | | | ISSVQAEDLAIYYCQQYYDT |
| | | | | PYTFGAGTKLELK |
| 59 | chAb11 | CDR-L1 | Residues 24-40 | RSSQSLLSSGNQKNYLA |
| | | | of SEQ ID NO.: 58 | |
| 45 | chAb11 | CDR-L2 | Residues 56-62 | WASTRQS |
| | | | of SEQ ID NO.: 58 | |
| 46 | chAb11 | CDR-L3 | Residues 95-103 | QQYYDTPYT |
| | | | of SEQ ID NO.: 58 | |
| 60 | chAb12 | VH | | EVQLVESGGGLVQPGRSLKL |
| | | | | SCAASGFTFSDYAMAWVRQA |
| | | | | PKKGLEWVASIIYDGRGTYY |
| | | | | RDSVKGRFTISRDNAKSTLY |
| | | | | LQMDSLRSEDTATYYCARQG |
| | | | | DGTYYYWGSFDYWGQGVMVT |
| | | | | VSS |
| 40 | chAb12 | CDR-H1 | Residues 26-35 | GFTFSDYAMA |
| | | | of SEQ ID NO.: 60 | |
| 41 | chAb12 | CDR-H2 | Residues 50-66 | SIIYDGRGTYYRDSVKG |
| | | | of SEQ ID NO.: 60 | |
| 61 | chAb12 | CDR-H3 | Residues 99-112 | QGDGTYYYWGSFDY |
| | | | of SEQ ID NO.: 60 | |
| 62 | chAb12 | VL | | DIVMTQSPSSLAVSAGETVT |
| | | | | INCKSSQSLLSSGNQKNYLA |
| | | | | WYQQKPGQSPKLLIYWASTR |
| | | | | QSGVPDRFIGSGSGTDFTLT |
| | | | | ISSVQAEDLAIYHCQQYYDT |
| | | | | PYTFGAGTKLELK |
| 44 | chAb12 | CDR-L1 | Residues 24-40 | KSSQSLLSSGNQKNYLA |
| | | | of SEQ ID NO.: 62 | |
| 45 | chAb12 | CDR-L2 | Residues 56-62 | WASTRQS |
| | | | of SEQ ID NO.: 62 | |
| 46 | chAb12 | CDR-L3 | Residues 95-103 | QQYYDTPYT |
| | | | of SEQ ID NO.: 62 | |
| 63 | chAb13 | VH | | QVQLKESGPGLVQPSQTLSL |
| | | | | TCTVSGFSLSSYGVIWVRQP |
| | | | | PGKGLEWMGIIWANGNTNYN |
| | | | | SALKSRLSISRDTSKSQVYL |
| | | | | KMNSLQTEDTATYYCARHYY |
| | | | | DGTHYYGYFDYWGQGVMVTV |
| | | | | SS |
| 64 | chAb13 | CDR-H1 | Residues 26-35 | GFSLSSYGVI |
| | | | of SEQ ID NO.: 63 | |
| 65 | chAb13 | CDR-H2 | Residues 50-65 | IIWANGNTNYNSALKS |
| | | | of SEQ ID NO.: 63 | |
| 66 | chAb13 | CDR-H3 | Residues 98-111 | HYYDGTHYYGYFDY |
| | | | of SEQ ID NO.: 63 | |
| 67 | chAb13 | VL | | DTVMTQTPSSQAVSAGEKVT |
| | | | | MSCKSSQSLLYSENKKKYLA |
| | | | | WYQQKPGQSPKLLIYWASTR |
| | | | | ESGVPDRFIGSGSGTDFTLT |
| | | | | ISSVQAEDLAVYYCQQYYNF |
| | | | | PYTFGAGTKLELK |
| 68 | chAb13 | CDR-L1 | Residues 24-40 | KSSQSLLYSENKKKYLA |
| | | | of SEQ ID NO.: 67 | |
| 7 | chAb13 | CDR-L2 | Residues 56-62 | WASTRES |
| | | | of SEQ ID NO.: 67 | |
| 69 | chAb13 | CDR-L3 | Residues 95-103 | QQYYNFPYT |
| | | | of SEQ ID NO.: 67 | |
| 70 | chAb14 | VH | | EVKLQQSGDELVRPGASVKI |
| | | | | SCKASGYTFTSYSMHWVKER |
| | | | | PGQGLEWIGAIFPIIGTTEY |
| | | | | NQKFKGKATLTADKSSNTAN |
| | | | | MELSRLTSEDSAVYYCARVY |
| | | | | LSYFDYWGQGVMVTVSS |
| 71 | chAb14 | CDR-H1 | Residues 26-35 | GYTFTSYSMH |
| | | | of SEQ ID NO.: 70 | |
| 72 | chAb14 | CDR-H2 | Residues 50-66 | AIFPIIGTTEYNQKFKG |
| | | | of SEQ ID NO.: 70 | |
| 73 | chAb14 | CDR-H3 | Residues 99-106 | VYLSYFDY |
| | | | of SEQ ID NO.: 70 | |
| 74 | chAb14 | VL | | DIQMTQSPSFLSASVGDRVT |
| | | | | INCKASQNINKYLDKYQRKH |
| | | | | GEAPKLLIYNTNNLQTGIPS |
| | | | | RFSGSGSGTDYTLTISSLQP |
| | | | | EDVATYFCLQHSSRYTFGAG |
| | | | | TKLELK |
| 75 | chAb14 | CDR-L1 | Residues 24-34 | KASQNINKYLD |
| | | | of SEQ ID NO.: 74 | |
| 76 | chAb14 | CDR-L2 | Residues 50-56 | NTNNLQT |
| | | | of SEQ ID NO.: 74 | |
| 77 | chAb14 | CDR-L3 | Residues 89-96 | LQHSSRYT |
| | | | of SEQ ID NO.: 74 | |
| 78 | chAb15 | | | EVQLVESGGGLVQPGRSLKL |
| | | | | SCAASGFTFSDYTMAWVRQA |
| | | | | PKKGLEWVATIIYDGRGTYY |
| | | | | RDSVKGRFTISRDNAKSTLY |
| | | | | LQMDSLRSEDTATYYCARQS |
| | | | | DGTYYYWGYFDYWGQGVMVT |
| | | | | VSS |
| 79 | chAb15 | CDR-H1 | Residues 26-35 | GFTFSDYTMA |
| | | | of SEQ ID NO.: 78 | |
| 80 | chAb15 | CDR-H2 | Residues 50-66 | TIIYDGRGTYYRDSVKG |
| | | | of SEQ ID NO.: 78 | |
| 81 | chAb15 | CDR-H3 | Residues 99-112 | QSDGTYYYWGYFDY |
| | | | of SEQ ID NO.: 78 | |
| 82 | chAb15 | VL | | DIVMTQSPSSLAVSAGETVT |
| | | | | INCKSSQSLLFSGNQKNYLA |
| | | | | WYQQKPGQSPKLLIYWASTR |
| | | | | QSGVPDRFIGSGSGTDFTLT |
| | | | | IRSVQAEDLAIYYCQQYYDS |
| | | | | PYTFGAGTKLELK |
| 83 | chAb15 | CDR-L1 | Residues 24-40 | KSSQSLLFS |
| | | | of SEQ ID NO.: 82 | GNQKNYLA |
| 45 | chAb15 | CDR-L2 | Residues 56-62 | WASTRQS |
| | | | of SEQ ID NO.: 82 | |
| 84 | chAb15 | CDR-L3 | Residues 95-103 | QQYYDSPYT |
| | | | of SEQ ID NO.: 82 | |

### Example 8. In vitro Binding Activity of Recombinant Anti-CD98 Chimeric Antibodies

The in vitro binding activities of the recombinant anti-CD98 chimeric mAbs were measured against both recombinant CD98 extracellular domain proteins (ECDs) and CD98-expressing cells.

Briefly, binding kinetics of anti-CD98 chimera mAbs for human and cynomolgus CD98 ECDs were determined by surface plasmon resonance-based measurements as described in Example 4. Table 10 reported the association and dissociation rate constants for CD98 binding, kₐ (M⁻¹s⁻¹) and k_{d} (s⁻¹), and the equilibrium dissociation constant K_{D} (M) of the interaction between antibodies and the target antigen. Binding of anti-CD98 chimera mAbs for CD98 on cell surface was evaluated by flow cytometry against CHO-K1 cell line stably transfected to express human CD98 and 3T12 cell line stably transfected to express cynomolgus monkey CD98. Data was analyzed using Graphpad Prism software and EC₅₀ values were reported as the concentration of antibody to achieve 50% of maximal binding to CD98-expressing cells (Table 10).

TABLE 10
Binding of Anti-CD98 Chimera mAbs to Human and Cynomolgus Monkey CD98.

| | Kinetics on Biacore | | | | | | Flow cytometry | |
|---|---|---|---|---|---|---|---|---|
| | huCD98 ECD | | | cyCD98 ECD | | | huCD98 | cyCD98 |
| Chimera | kₐ | k_{d} | K_{D} | kₐ | k_{d} | K_{D} | EC₅₀ | EC₅₀ |
| mAb | (M⁻¹s⁻¹) | (s⁻¹) | (M) | (M⁻¹s⁻¹) | (s⁻¹) | (M) | (nM) | (nM) |
| chAb1 | 6.8E+04 | 1.5E−05 | 2.1E−10 | 2.8E+04 | 2.2E−05 | 8.0E−10 | 1.67 | 9.86 |
| chAb2 | 5.9E+04 | 9.6E−05 | 1.6E−09 | 1.9E+04 | 1.1E−03 | 5.8E−08 | 3.84 | 12.68 |
| chAb3 | 1.6E+05 | 3.4E−03 | 2.1E−08 | 3.9E+04 | 1.6E−03 | 4.0E−08 | 2.02 | 9.87 |
| chAb4 | 2.8E+04 | 7.8E−05 | 2.8E−09 | 8.8E+03 | 7.3E−05 | 8.3E−09 | 5.41 | 17.21 |
| chAb5 | 4.0E+04 | 3.0E−04 | 7.5E−09 | 1.8E+04 | 4.4E−04 | 2.5E−08 | 3.25 | 24.23 |
| chAb6 | 1.1E+05 | 1.4E−04 | 1.3E−09 | 4.7E+04 | 3.7E−04 | 7.9E−09 | 2.08 | 5.99 |
| chAb7 | 1.4E+05 | 6.2E−05 | 4.5E−10 | 6.3E+04 | 2.2E−04 | 3.5E−09 | 2.56 | 6.49 |
| chAb8 | 6.7E+04 | 2.8E−04 | 4.2E−09 | 2.8E+04 | 5.0E−04 | 1.7E−08 | 2.61 | 10.13 |
| chAb9 | 7.3E+04 | 2.4E−04 | 3.3E−09 | 3.3E+04 | 6.3E−04 | 1 9E−08 | 8.55 | 11.12 |
| chAb10 | 1.0E+05 | 3.3E−04 | 3.3E−09 | 5.9E+04 | 6.4E−04 | 1.1E−08 | 6.45 | 10.79 |
| chAb11 | 4.5E+04 | 1.3E−03 | 2.8E−08 | 1.8E+04 | 2.0E−03 | 1.1E−07 | 4.57 | 16.95 |
| chAb12 | 5.4E+04 | 6.3E−05 | 1.2E−09 | 2.4E+04 | 9.3E−05 | 3.8E−09 | 2.89 | 14.91 |
| chAbl 3 | 1.3E+05 | 1.6E−04 | 1.2E−09 | 6.2E+04 | 4.4E−04 | 7.1E−09 | 3.24 | 2.98 |
| chAb14 | 2.2E+04 | 8.4E−05 | 3.9E−09 | 1.2E+04 | 1.4E−03 | 1.1E−07 | 3.75 | 17.99 |
| chAb15 | 4.3E+04 | 4.6E−05 | 1.1E−09 | 2.0E+04 | 7.4E−05 | 3.7E−09 | 6.63 | 20.3 |

hu = human;
cy = cynomolgus monkey;
ECD = extracellular domain;
E + Y = ×10^{Y};
E − Y = ×10^{-Y}.

### Example 9. In Vitro Potency of Bcl-xL Inhibitor ADCs Derived from Anti-CD98 Chimeric Antibodies

Ten selected anti-CD98 chimeric mAbs were first conjugated in a small-scale (ranging from 0.5 to 2 mg) with the Bcl-xL inhibitor synthon CZ according to Method A, as described in Example 5. The activity of these ADCs were tested in growth inhibition assays in three human cancer cell lines expressing endogenous CD98, NCI—H146 small cell lung cancer line, H2170 non-small cell lung cancer line, and Molt-4 human acute lymphoblastic leukemia cell line. Briefly, 3000 cells per well were plated into 96-well plates and were treated with ADC in serial dilution. After 4 days, the number of viable cells was determined by the CellTiter-Glo® reagent (Promega G7572) as instructed by the manufacturer. Data was analyzed using Graphpad Prism software and IC₅₀ values were reported as the concentration of ADC to achieve 50% inhibition of cell proliferation (Table 11).

TABLE 11
In vitro Potency of Bcl-xL inhibitor ADCs Conjugated
from Anti-CD98 Chimeric Antibodies.

| | Dar | % | ADC potency, IC₅₀ (nM) | | |
|---|---|---|---|---|---|
| | by | Aggregates | H146 | H2170 | MOLT4 |
| ADC | MS | by SEC | (SCLC) | (NSCLC) | (leukemia) |
| ChAb1 | 1.21 | 2.75 | ~0.196 | 0.276 | 0.071 |
| ChAb3 | 3.08 | 3.95 | 0.255 | ~0.171 | 0.026 |
| ChAb4 | 2.88 | 4.21 | 0.043 | 0.120 | 0.030 |
| ChAb5 | 2.44 | 3.58 | ~0.188 | 0.161 | 0.029 |
| ChAb8 | 3.93 | 9.29 | 0.094 | 0.112 | 0.038 |
| ChAb10 | 3.09 | 3.02 | 0.209 | 0.090 | 0.035 |
| ChAb11 | 3.30 | 5.08 | 0.527 | 0.195 | 0.199 |
| ChAb12 | 3.54 | 4.05 | 0.242 | 0.118 | 0.034 |
| ChAb13 | 2.74 | 1.89 | 0.115 | 0.321 | 0.052 |
| ChAb15 | 3.62 | 3.6 | 0.194 | 0.137 | 0.048 |
| *MSL109-CZ | 4.5 | 2.7 | >200 | >200 | >200 |

DAR = drugs/antibody ratio;
MS = Mass spectrometry;
SEC = Size exclusion chromatography
*MSL109 is a humanized IgG1 antibody that binds to cytomegalovirus (CMV) glycoprotein H. It is used as a negative control mAb.

### Example 10. In Vitro Potency of Bcl-xL Inhibitor ADC Purified to Contain Homogenous DAR2 or DAR4 Species

To evaluate the potency of Bcl-xL inhibitor ADC containing homogenous DAR2 (also referred to as “E2”) and DAR4 (also referred to as “E4”) species, anti-CD98 chimeric chAb3 was conjugated with the Bcl-xL inhibitor payload CZ to a broad DAR4.1, followed by hydrophobic interaction chromatography (HIC) purification to prepare DAR2 and DAR4 species according to the Methods noted in Table 10. The activity of these HIC purified DAR species were tested in growth inhibition assays in three human cancer cell lines expressing CD98, EBC-1 non-small cell lung cancer line, H2170 non-small cell lung cancer line, and Molt-4 human acute lymphoblastic leukemia cell line. After 3-4 days, the number of viable cells was determined by the CellTiter-Glo® reagent (Promega G7572) as instructed by the manufacturer. Data was analyzed using Graphpad Prism software and IC₅₀ values were reported as the concentration of ADC to achieve 50% inhibition of cell proliferation (Table 12).

TABLE 12
In vitro Potency of Bcl-xL inhibitor ADCs Conjugated
from Anti-CD98 Chimeric Antibodies

| | | | % | ADC potency, IC₅₀ (nM) | | |
|---|---|---|---|---|---|---|
| | Synthetic | Dar by | Aggregates | EBC | H2170 | MOLT4 |
| ADC | Method | MS | by SEC | (NSCLC) | (NSCLC) | (leukemia) |
| ChAb3-CZ-DAR2 | H | 2.0 | 0.5 | 271 | 0.273 | 0.046 |
| ChAb3-CZ-DAR4 | H | 4.0 | 13.8 | 10.39 | 0.225 | 0.021 |
| ChAb3-CZ-Broad | A | 4.1 | 12.2 | 7.15 | 0.150 | 0.034 |
| DAR | | | | | | |
| *MSL109-CZ | A | 4.5 | 2.7 | >200 | >200 | >200 |

DAR = drugs/antibody ratio;
MS = Mass spectrometry;
SEC = Size exclusion chromatography
*MSL109 is a humanized IgG1 antibody that binds to cytomegalovirus (CMV) glycoprotein H. It is used as a negative control mAb.

### Example 11. In vivo Efficacy of Anti-CD98 Chimera mAb ADCs

The anti-CD98 chimera mAbs were then conjugated to the Bcl-xL inhibitor synthon CZ according to the Methods noted in Table 13, and described in Example 5, and their in vivo efficacy was evaluated in EBC-1 (human lung squamous cell carcinoma) xenograft model. EBC-1 was obtained from the Japanese Collection of Research Bioresources Cell Bank (JCRB, Osaka, Japan) and were cultured using MEM culture media with 10% FBS. To generate xenografts, 5×10⁶ EBC-1 viable cells were inoculated subcutaneously into the right flank of immune deficient female SCID-bg mice (Charles River Laboratories. Wilmington, Mass.). The injection volume was 0.2 mL and the inoculum was composed of 1:1 mixture of S-MEM and Matrigel. Tumors were size matched at approximately 200 mm³. Antibodies and conjugates were formulated in phosphate buffered saline. pH 7.2 and injected intraperitoneally. Injection volume did not exceed 200 μl. Therapy began within 24 hours after size matching of the tumors. Mice weighed approximately 21-22 g at the onset of therapy. Tumor volume was estimated two to three times weekly. Measurements of the length (L) and width (W) of the tumor were taken via electronic caliper and the volume was calculated according to the following equation: V=L×W²/2. Mice were euthanized when tumor volume reached 3,000 mm³ or skin ulcerations occurred. Eight mice were housed per cage. Food and water were available ad libitum. Mice were acclimated to the animal facilities for a period of at least one week prior to commencement of experiments. Animals were tested in the light phase of a 12-hour light: 12-hour dark schedule (lights on at 06:00 hours). Anti-CD98 conjugates (10 mg/kg) were administered as a single dose (QDx1) intraperitoneally. A human IgG control antibody (AB095, a human IgG1 antibody recognizing tetanus toxoid See Larrick et al., 1992, Immunological Reviews 69-85) was used as a negative control agent.

To refer to efficacy of therapeutic agents, parameters of amplitude (TGIₘₐₓ), durability (TGD) and response frequency (CR, PR, OR) of therapeutic response are used as described in the Example 6. The efficacy of inhibition of EBC-1 xenografts growth with CD98-targeted ADCs is illustrated by Table 13, below.

TABLE 13
Inhibition of EBC-1 xenograft tumor growth after treatment
with a single dose of CD98-targeting Bcl-xLi ADC

| *ADC | | Dose^{[a]}/ | Growth Inhibition | | Response Frequency | | |
|---|---|---|---|---|---|---|---|
| DAR/ | | route/ | TGIₘₐₓ | TGD | CR | PR | OR |
| Method | Treatment | regimen | (%) | (%) | (%) | (%) | (%) |
| | AB095** | 10/IP/QDx1 | 0 | 0 | 0 | 0 | 0 |
| 4.0/A | ChAb8-CZ | 10/IP/QDx1 | 56* | 42* | 0 | 0 | 0 |
| 3.8/A | ChAb11-CZ | 10/IP/QDx1 | 55* | 37* | 0 | 0 | 0 |
| 4.1/A | ChAb12-CZ | 10/IP/QDx1 | 76* | 74* | 0 | 25 | 25 |
| 4.0/A | ChAb13-CZ | 10/IP/QDx1 | 61* | 47* | 0 | 0 | 0 |
| 4.4/A | ChAb15-CZ | 10/IP/QDx1 | 70* | 74* | 0 | 0 | 0 |
| 4.1/A | ChAb3-CZ | 10/IP/QDx1 | 54* | 37* | 0 | 0 | 0 |
| 4.1/A | ChAb4-CZ | 10/IP/QDx1 | 63* | 37* | 0 | 0 | 0 |
| 4.5/A | ChAb5-CZ | 10/IP/QDx1 | 70* | 61* | 0 | 0 | 0 |
| 2/H | ChAb3-CZ DAR2 | 10/IP/QDx1 | 55* | 32* | 0 | 0 | 0 |

*DAR = drugs/antibody ratio as determined by mass spectrometry; Method refers to protocol used for the generation of the ADC sample (see Example 5, above).
**Negative control IgG1 mAb binding to tetanus toxin.
^{[a]}dose is given in mg/kg/day.
*= p < 005 as compared to control treatment (AB095).

### Example 12. Humanization of ChAb3 and ChAb15 Anti-CD98 Chimera mAbs

Antibodies chAb3 and chAb15 were chosen for humanization and additional modification based on their favorable properties as Bcl-xL inhibitor conjugates.

ChAb3 and chAb15 were humanized by applying the method of CDR-grafting. Humanized antibodies were generated based on the variable heavy (VH) and variable light (VL) CDR sequences of chAb3 and chAb15. Specifically, human germline sequences were selected for constructing CDR-grafted, humanized chAb3 and chAb15 antibodies where the CDR domains of the VH and VL chains of chAb3 and chAb15 were grafted onto different human heavy and light chain acceptor sequences:

1, chAb3 Humanization

Based on the alignments with the VH and VL sequences of monoclonal antibody chAb3 of the present invention, the following known human sequences are selected:
IGHV3-49*03 and IGHJ1*01 for constructing heavy chain acceptor sequences
IGHV3-15*01 and IGHJ1*01 as backup acceptor for constructing heavy chain
IGHV3-72*01 and IGHJ1*01 as backup acceptor for constructing heavy chain
IGKV4-1*01 and IGKJ4*01 for constructing light chain acceptor sequences
IGKV2-40*01 and IGKJ4*01 as backup acceptor for constructing light chain

By grafting the corresponding VH and VL CDRs of chAb3 into corresponding acceptor sequences, the CDR-grafted, humanized, and modified VH and VL sequences were prepared. Furthermore, to generate humanized antibody with potential framework back-mutations, the mutations were identified and introduced into the CDR-grafted antibody sequences by de novo synthesis of the variable domain, or mutagenic oligonucleotide primers and polymerase chain reactions, or both by methods well known in the art. Different combinations of back mutations and other mutations are constructed for each of the CDR-grafts as follows. Residue numbers for these mutations are based on the Kabat numbering system.

For heavy chains hCL-chAb3VH.1, one or more of the following Vernier and VH/VL interfacing residues were back mutated as follows: Q3->K. F37->V, V48->L, A78->L. Additional mutations include the following: A24->T, D73->N.

For heavy chains hCL-chAb3VH.2, one or more of the following Vernier and VH/VL interfacing residues were back mutated as follows: Q3->K. V48->L. Additional mutations include the following: A24->T, D73->N. N76->S, T77->I, T94->R.

For heavy chains hCL-chAb3VH.3, one or more of the following Vernier and VH/VL interfacing residues were back mutated as follows: Q3->K. V48->L, A93->T. Additional mutations include the following: A24->T. D73->N, N76->S, S77->I.

For light chains hCL-chAb3VL.1, one or more of the following Vernier and VH/VL interfacing residues were back mutated as follows: P43->S

For light chains hCL-chAb3VL.2, no residues were back mutated.

The following humanized variable regions of the murine monoclonal chAb3 antibodies were cloned into IgG expression vectors for functional characterization (Table 14).

TABLE 14
Sequences of chAb3 humanized variable regions.

| | Protein | |
|---|---|---|
| SEQ ID NO: | Sequence | region |
| 130 | hCL-Ab3VH.1 | EVQLVESGGGLVQPGRSLRLSCTASGFTFIDYYMSWF |
| | | RQAPGKGLEWVGFIRNKANGYTTEYSASVKGRFTISR |
| | | DDSKSIAYLQMNSLKTEDTAVYYCTRDRPAWFVYWGQ |
| | | GTLVTVSS |
| 5 | hCL-Ab3VH.1a | EVQLVESGGGLVQPGRSLRLSCTTSGFTFIDYYMSWV |
| | | RQAPGKGLEWLGFIRNKANGYTTEYSASVKGRFTISR |
| | | DNSKSILYLQMNSLKTEDTAVYYCTRDRPAWFVYWGQ |
| | | GTLVTVSS |
| 131 | hCL-Ab3VH.1b | EVKLVESGGGLVQPGRSLRLSCTASGFTFIDYYMSWV |
| | | RQAPGKGLEWLGFIRNKANGYTTEYSASVKGRFTISR |
| | | DDSKSILYLQMNSLKTEDTAVYYCTRDRPAWFVYWGQ |
| | | GTLVTVSS |
| 132 | hCL-Ab3VH.1c | EVKLVESGGGLVQPGRSLRLSCTASGFTFIDYYMSWF |
| | | RQAPGKGLEWLGFIRNKANGYTTEYSASVKGRFTISR |
| | | DDSKSIAYLQMNSLKTEDTAVYYCTRDRPAWFVYWGQ |
| | | GTLVTVSS |
| 133 | hCL-Ab3VH.2 | EVQLVESGGGLVKPGGSLRLSCAASGFTFIDYYMSWV |
| | | RQAPGKGLEWVGFIRNKANGYTTEYSASVKGRFTISR |
| | | DDSKNTLYLQMNSLKTEDTAVYYCTTDRPAWFVYWGQ |
| | | GTLVTVSS |
| 134 | hCL-Ab3VH.2a | EVQLVESGGGLVKPGGSLRLSCATSGFTFIDYYMSWV |
| | | RQAPGKGLEWLGFIRNKANGYTTEYSASVKGRFTISR |
| | | DNSKSILYLQMNSLKTEDTAVYYCTRDRPAWFVYWGQ |
| | | GTLVTVSS |
| 135 | hCL-Ab3VH.2b | EVKLVESGGGLVKPGGSLRLSCAASGFTFIDYYMSWV |
| | | RQAPGKGLEWLGFIRNKANGYTTEYSASVKGRFTISR |
| | | DDSKNTLYLQMNSLKTEDTAVYYCTTDRPAWFVYWGQ |
| | | GTLVTVSS |
| 136 | hCL-Ab3VH.3 | EVQLVESGGGLVQPGGSLRLSCAASGFTFIDYYMSWV |
| | | RQAPGKGLEWVGFIRNKANGYTTEYSASVKGRFTISR |
| | | DDSKNSLYLQMNSLKTEDTAVYYCARDRPAWFVYWGQ |
| | | GTLVTVSS |
| 137 | hCL-Ab3VH.3a | EVQLVESGGGLVQPGGSLRLSCATSGFTFIDYYMSWV |
| | | RQAPGKGLEWLGFIRNKANGYTTEYSASVKGRFTISR |
| | | DNSKSILYLQMNSLKTEDTAVYYCTRDRPAWFVYWGQ |
| | | GTLVTVSS |
| 138 | hCL-Ab3VH.3b | EVKLVESGGGLVQPGGSLRLSCAASGFTFIDYYMSWV |
| | | RQAPGKGLEWLGFIRNKANGYTTEYSASVKGRFTISR |
| | | DDSKNSLYLQMNSLKTEDTAVYYCTRDRPAWFVYWGQ |
| | | GTLVTVSS |
| 139 | hCL-Ab3VH.3c | EVKLVESGGGLVQPGGSLRLSCAASGFTFIDYYMSWV |
| | | RQAPGKGLEWLGFIRNKANGYTTEYSASVKGRFTISR |
| | | DDSKNSLYLQMNSLKTEDTAVYYCARDRPAWFVYWGQ |
| | | GTLVTVSS |
| 140 | hCL-Ab3VL.1 | DIVMTQSPDSLAVSLGERATINCKSSQSLLYSSNQKN |
| | | YLAWYQQKPGQPPKLLIYWASTRESGVPDRFSGSGSG |
| | | TDFTLTISSLQAEDVAVYYCQQYYSYPYTFGGGTKVE |
| | | IK |
| 141 | hCL-Ab3VL.la | DIVMTQSPDSLAVSLGERATINCKSSQSLLYSSNQKN |
| | | YLAWYQQKPGQSPKLLIYWASTRESGVPDRFSGSGSG |
| | | TDFTLTISSLQAEDVAVYYCQQYYSYPYTFGGGTKVE |
| | | IK |
| 142 | hCL-Ab3VL.2 | DIVMTQTPLSLPVTPGEPASISCKSSQSLLYSSNQKN |
| | | YLAWYLQKPGQSPQLLIYWASTRESGVPDRFSGSGSG |
| | | TDFTLKISRVEAEDVGVYYCQQYYSYPYTFGGGTKVE |
| | | IK |

*hCL-Ab3VH.1 is a CDR-grafted, humanized chAb3 VH containing IGHV3-49*03 and IGHJ1*01 framework sequences.
*hCL-Ab3VH.1a is a humanized design based on .1 and contains 5 proposed framework back-mutation(s): A24T, F37V, V48L, D73N, A78L.
*hCL-Ab3VH.1b is an intermediate design between on .1 and 1a and contains 4 proposed framework back-mutation(s): Q3K, F37V, V48L, A78L.
*hCL-Ab3VH.1c is a design based on .1b with the elimination of Carter residue back-mutations. It contains 2 proposed framework back-mutation(s): Q3K, V48L.
*hCL-Ab3VH.2 is a CDR-grafted, humanized chAb3 VH containing IGHV3-15*01 and IGHJ1*01 framework sequences.
*hCL-Ab3VH.2a is a humanized design based on .2 and contains 6 proposed framework back-mutation(s): A24T, V48L, D73N, N76S, T77I, T94R.
*hCL-Ab3VH.2b is an intermediate design between on .2 and .2a and contains 2 proposed framework back-mutation(s): Q3K, V48L.
*hCL-Ab3VH.3 is a CDR-grafted, humanized chAb3 VH containing IGHV3-72*01 and IGHJ1*01 framework sequences.
*hCL-Ab3VH.3a is a humanized design based on .3 and contains 6 proposed framework back-mutation(s): A24T, V48L, D73N, N76S, S77I, A93T.
*hCL-Ab3VH.3b is an intermediate design between on .3 and .3a and contains 3 proposed framework back-mutation(s): Q3K, V48L, A93T.
*hCL-Ab3VH.3c is a design based on .3b with the elimination of Carter residue back-mutations. It contains 2 proposed framework back-mutation(s): Q3K, V48L.
*hCL-Ab3VL.1 is a CDR-grafted, humanized chAb3 VL containing IGKV4-1*01 and IGKJ4*01 framework sequences.
*hCL-Ab3VL.1a is a humanized design based on .1 and contains 1 proposed framework back-mutation(s): P43S.
*hCL-Ab3VL.2 is a CDR-grafted, humanized chAb3 VL containing IGKV2-40*01 and IGKJ4*01 framework sequences.

2, chAb15S humanization

Based on the alignments with the VH and VL sequences of monoclonal antibody chAb15 of the present invention, the following known human sequences were selected:
IGHV3-30*01(0-1) and IGHJ3*01 for constructing heavy chain acceptor sequences
IGHV3-7*01 and IGHJ3*01 as backup acceptor for constructing heavy chain
IGHV1-46*01 and IGHJ3*01 as backup acceptor for constructing heavy chain
IGKV4-1*01 and IGKJ2*01 for constructing light chain acceptor sequences
IGKV2-40*01 and IGKJ2*01 as backup acceptor for constructing light chain

By grafting the corresponding VH and VL CDRs of chAb15 into corresponding acceptor sequences, the CDR-grafted, humanized, and modified VH and VL sequences were prepared. Furthermore, to generate humanized antibody with potential framework back-mutations, the mutations were identified and introduced into the CDR-grafted antibody sequences by de novo synthesis of the variable domain, or mutagenic oligonucleotide primers and polymerase chain reactions, or both by methods well known in the art. Different combinations of back mutations and other mutations are constructed for each of the CDR-grafts as follows. Residue numbers for these mutations are based on the Kabat numbering system.

For heavy chains hCL-Ab15VH.1, one or more of the following Vernier and VH/VL interfacing residues were back mutated as follows: S77->T

For heavy chains hCL-Ab15VH.2, one or more of the following Vernier and VH/VL interfacing residues were back mutated as follows: M48->V. V67->F, M69->T, T73->N, V78->L. Additional mutations include the following: Q1->E, G49->A. M80->L.

For light chains hCL-Ab15VL.1, one or more of the following Vernier and VH/VL interfacing residues were back mutated as follows: P43->S, V85->I

For light chains hCL-Ab15VL.2, one or more of the following Vernier and VH/VL interfacing residues were back mutated as follows: S22->N, V85->I

The following humanized variable regions of the murine monoclonal chAb15 antibodies were cloned into TgG expression vectors for functional characterization (Table 15).

TABLE 15
Sequences of chAb15 humanized variable regions.

| | Protein | |
|---|---|---|
| SEQ ID NO: | Sequence | region |
| 143 | hCL-Ab15VH.1z | QVQLVESGGGVVQPGRSLRLSCAASgftfsdytmaW |
| | | VRQAPGKGLEWVAtiiydgrgtyyrdsvkgRFTISR |
| | | DNSKNTLYLQMNSLRAEDTAVYYCARqsdgtyyywg |
| | | yfdyWGQGTMVTVSS |
| 144 | hCL-Ab15VH.1 | EVQLVESGGGVVQPGRSLRLSCAASGFTFSDYTMAW |
| | | VRQAPGKGLEWVATIIYDGRGTYYRDSVKGRFTISR |
| | | DNSKNTLYLQMNSLRAEDTAVYYCARQSDGTYYYWG |
| | | YFDYWGQGTMVTVSS |
| 122 | hCL-Ab15VH.1a | EVQLVESGGGVVQPGRSLRLSCAASgftfsdytmaW |
| | | VRQAPGKGLEWVAtiiydgrgtyyrdsvkgRFTISR |
| | | DNSKSTLYLQMNSLRAEDTAVYYCARqsdgtyyywg |
| | | yfdyWGQGTMVTVSS |
| 145 | hCL-Ab15VH.2 | EVQLVESGGGLVQPGGSLRLSCAASgftfsdytmaW |
| | | VRQAPGKGLEWVAtiiydgrgtyyrdsvkgRFTISR |
| | | DNAKNSLYLQMNSLRAEDTAVYYCARqsdgtyyywg |
| | | yfdyWGQGTMVTVSS |
| 146 | hCL-Ab15VH.2a | EVQLVESGGGLVQPGGSLRLSCAASgftfsdytmaW |
| | | VRQAPGKGLEWVAtiiydgrgtyyrdsvkgRFTISR |
| | | DNAKNTLYLQMNSLRAEDTAVYYCARqsdgtyyywg |
| | | yfdyWGQGTMVTVSS |
| 147 | hCL-Ab15VH.3z | QVQLVQSGAEVKKPGASVKVSCKASgftfsdytmaW |
| | | VRQAPGQGLEWMGtiiydgrgtyyrdsvkgRVTMTR |
| | | DTSTSTVYMELSSLRSEDTAVYYCARqsdgtyyywg |
| | | yfdyWGQGTMVTVSS |
| 148 | hCL-Ab15VH.3 | EVQLVQSGAEVKKPGASVKVSCKASgftfsdytmaW |
| | | VRQAPGQGLEWMGtiiydgrgtyyrdsvkgRVTMTR |
| | | DTSTSTVYMELSSLRSEDTAVYYCARqsdgtyyywg |
| | | yfdyWGQGTMVTVSS |
| 149 | hCL-Ab15VH.3a | EVQLVQSGAEVKKPGASVKVSCKASgftfsdytmaW |
| | | VRQAPGQGLEWVAtiiydgrgtyyrdsvkgRFTITR |
| | | DNSTSTLYLELSSLRSEDTAVYYCARqsdgtyyywg |
| | | yfdyWGQGTMVTVSS |
| 150 | hCL-Ab15VH.3b | EVQLVQSGAEVKKPGASVKVSCKASgftfsdytmaW |
| | | VRQAPGQGLEWVGtiiydgrgtyyrdsvkgRFTITR |
| | | DNSTSTLYMELSSLRSEDTAVYYCARqsdgtyyywg |
| | | yfdyWGQGTMVTVSS |
| 151 | hCL-Ab15VL.1 | DIVMTQSPDSLAVSLGERATINCkssqsllfsgnqk |
| | | nylaWYQQKPGQPPKLLIYwastrqsGVPDRFSGSG |
| | | SGTDFTLTISSLQAEDVAVYYCqqyydspytFGQGT |
| | | KLEIK |
| 123 | hCL-Ab15VL.1a | DIVMTQSPDSLAVSLGERATINCkssqsllfsgnqk |
| | | nylaWYQQKPGQSPKLLIYwastrqsGVPDRFSGSG |
| | | SGTDFTLTIRSLQAEDVAIYYCqqyydspytFGQGT |
| | | KLEIK |
| 152 | hCL-Ab15VL.2 | DIVMTQTPLSLPVTPGEPASISCkssqsllfsgnqk |
| | | nylaWYLQKPGQSPQLLIYwastrqsGVPDRFSGSG |
| | | SGTDFTLKISRVEAEDVGVYYCqqyydspytFGQGT |
| | | KLEIK |
| 153 | hCL-Ab15VL.2a | DIVMTQTPLSLPVTPGEPASINCkssqsllfsgnqk |
| | | nylaWYLQKPGQSPQLLIYwastrqsGVPDRFSGSG |
| | | SGTDFTLKISRVEAEDVGIYYCqqyydspytFGQGT |
| | | KLEIK |

*hCLAb15VH.1z is a CDR-grafted, humanized chAb15 VH containing IGHV3-30*01(0-1) and IGHJ3*01 framework sequences.
*hCLAb15VH.1 is based on 1z with a Q1E change to prevent pyroglutamate formation.
*hCLAb15VH.1a is a humanized design based on .1 and contains 1 proposed framework back-mutation(s): N76S.
*hCL-Ab15VH.2 is a CDR-grafted, humanized chAb15 VH containing IGHV3-7*01 and IGHJ3*01 framework sequences.
*hCL-Ab15VH.2a is a humanized design based on .1 and contains 1 proposed framework back-mutation(s): S77T.
*hCL-Ab15VH.3z is a CDR-grafted, humanized chAb15 VH containing IGHV1-46*01 and IGHJ3*01 framework sequences.
*hCL-Ab15VH.3 is based on .3z with a Q1E change to prevent pyroglutamate formation.
*hCL-Ab15VH.3a is a humanized design based on .3 and contains 7 proposed framework back-mutation(s): M48V, G49A, V67F, M69I, T73N, V78L, M80L.
*hCL-Ab15VH.3b is an intermediate design between on .3 and .3a and contains 5 proposed framework back-mutation(s): M48V, V67F, M691, T73N, V78L.
*hCLAb15VL.1 is a CDR-grafted, humanized chAb15 VL containing IGKV4-1*01 and IGKJ2*01 framework sequences.
*hCLAb15VL.1a is a humanized design based on .1 and contains 3 proposed framework back-mutation(s): P43S, S76R, V85I.
*hCL-Ab15VL.2 is a CDR-grafted, humanized chAb15 VL containing IGKV2-40*01 and IGKJ2*01 framework sequences.
*hCL-Ab15VL.2a is a humanized design based on .2 and contains 2 proposed framework back-mutation(s): S22N, V85I.

Humanized chAb3 and humanized chAb15 are referred to herein as huAb3 and huAb15, respectively, and are set forth below in Table 16.

TABLE 16
Variable region sequences of huAb3 and huAb15

| SEQ | | | | |
|---|---|---|---|---|
| ID | | Protein | | |
| NO: | Clone | Region | Residues | V Region |
| 85 | huAb3 | VH | | EVQLVESGGGLVQPGRSLRLSCTTS |
| | | | | GETFIDYYMSWVRQAPGKGLEWLGF |
| | | | | IRNKANGYTTEYSASVKGRFTISRD |
| | | | | NSNSILYLQMNSLKTEDTAVYYCTR |
| | | | | DRPAWFVYWGQGTLVTVSS |
| 10 | huAb3 | CDR-H1 | Residues 26-35 | GFTFIDYYMS |
| | | | of SEQ ID NO.: 85 | |
| 11 | huAb3 | CDR-H2 | Residues 50-66 | FIRNKANGYTTEYSASVKG |
| | | | of SEQ ID NO.: 85 | |
| 17 | huAb3 | CDR-H3 | Residues 99-112 | DRPAWFVY |
| | | | of SEQ ID NO.: 85 | |
| 88 | huAb3 | VL | | DIVNTQSPDSLAVSLGERATINCKS |
| | | | | SQSLLYSSNQKNYLAWYQQKPGQSP |
| | | | | KLLIYWASTRESGVPDRFSGSGSGT |
| | | | | DFTLTISSLQAEDVAVYYCQQYYSY |
| | | | | PYTFGGGTKVEIK |
| 13 | huAb3 | CDR-L1 | Residues 24-40 | KSSQSLLYSSNQKNYLA |
| | | | of SEQ ID NO.: 88 | |
| 7 | huAb3 | CDR-12 | Residues 56-62 | WASTRES |
| | | | of SEQ ID NO.: 88 | |
| 19 | huAb3 | CDR-13 | Residues 95-103 | QQYYSYPYT |
| | | | of SEQ ID NO.: 88 | |
| 122 | huAb15 | VH | | EVQLVESGGGVVQPGRSLRLSCAAS |
| | | | | GETFSDYTMAWVRQAPGKGLEWVAT |
| | | | | IIYDGRGTYYRDSVKGRFTISRDNS |
| | | | | KSTLYLQMNSLRAEDTAVYYCARQS |
| | | | | DGTYYYWGYFDYWGQGTMVTVSS |
| 79 | huAb15 | CDR-H1 | Residues 26-35 | GFTESDYTMA |
| | | | of SEQ ID NO.: 122 | |
| 80 | huAb15 | CDR-H2 | Residues 50-66 | TIIYDGRGTYYRDSVKG |
| | | | of SEQ ID NO.: 122 | |
| 81 | huAb15 | CDR-H3 | Residues 99-112 | QSDGTYYYWGYTDY |
| | | | of SEQ ID NO.: 122 | |
| 123 | huAb15 | VL | | DIVMTQSPDSLAVSLGERATINCKS |
| | | | | SQSLLFSGNQKNYLAWYQQKPGQSP |
| | | | | KLLTYWASTRQSGVPDPTSGSGSGT |
| | | | | DFTLTIRSLQAEDVAIYYCQQYYDS |
| | | | | PYTITGQGTKLEIK |
| 83 | huAb15 | CDR-L1 | Residues 24-40 | KSSQSLLFSGNQKNYLA |
| | | | of SEQ ID NO.: 123 | |
| 45 | huAb15 | CDR-L2 | Residues 56-62 | WASTRQS |
| | | | of SEQ ID NO.: 123 | |
| 84 | huAb15 | CDR-L3 | Residues 95-103 | QQYYDSPYT |
| | | | of SEQ ID NO.: 123 | |

Additional Engineering of huAb3 and huAb5S

Further engineering of huAb3 and huAb15 was performed in order to identify and remove post-translational modifications that have the potential to reduce affinity, potency, stability and homogeneity of an antibody. These amino acid residues are identified below by bold, underlining in the variable regions of huAb3 and huAb15. The residues were removed by PCR. Variants were generated containing point mutations in of the identified amino acid including all possible amino acids except M, C, N, D, G, S, or P. All variants were expressed as full-length antibodies, and evaluated for CD98 binding. Humanized antibodies with these potentially adverse residues removed that maintained binding to human CD98 are listed in Table 17.

Humanized chAb3 (huAb3)
VH sequence: hCLAb3VH.1a
(SEQ ID NO: 85)
EVQINESGGGLVQPGRSLRLSCTTSgftfidyymsWVRQAPGNGLEWLGf
irnkangytteysasvkgRFTISRDNSKSILYLQMNSLKTEDTAVYYCTR
drpawfvyWGQGTLVTVSS
VL sequence: hCLAb3VL.1a
(SEQ ID NO: 88)
DIVMTQSPDSLAVSLGERATINCkssqsllyssnqknylaWYQQKPGQSP
RLLIYwastresGVPDRFSGSGSGTDFTLTISSLQAEDVAVYYCTqqyys
ypytFGGGTKVEIK
Humanized chAb15 (huAb15)
VH sequence: hCLAb15VE.1a
(SEQ ID NO: 122)
EVQLVESGGGVVQPGRSLRLSCAASgftfsdytmaMVRQAPGKGLEWVAt
iiydgrgtyyrdsvkgRFTISRDNSKSTLYLQMNSLRAEDTAVYYCARqs
dgtyyywgyfdyWGQGTMVTVSS
VL sequence: hCLAb15VL.1a
(SEQ ID NO: 123)
DIVMTQSPDSLAVSLGERATINCkssqsllfsgnqknylaWYQQKPGQSP
KLLIYwastrqsGVPDRFSGSGSGTDFTLTIRSLQAEDVAIYYCqqyyds
pytFGQGTKLEIK

TABLE 17
Humanized Clones Derived from
Chimera mAb chAb3 and chAb15

| Humanized | Parental | VH | VL |
|---|---|---|---|
| clone | chimera | framework | framework |
| huAb3v1 | chAb3 | IGHV3-49 | IGKV4-1 |
| huAb3v2 | chAb3 | IGHV3-49 | IGKV4-1 |
| huAb15v1 | chAb15 | IGHV3-30 | IGKV4-1 |
| huAb15v2 | chAb15 | IGHV3-30 | IGKV4-1 |
| huAb15v3 | chAb15 | IGHV3-30 | IGKV4-1 |
| huAb15v4 | chAb15 | IGHV3-30 | IGKV4-1 |
| huAb15v5 | chAb15 | IGHV3-30 | IGKV4-1 |
| huAb15v6 | chAb15 | IGHV3-30 | IGKV4-1 |
| huAb15v7 | chAb15 | IGHV3-30 | IGKV4-1 |

The VH and VL sequences of these further engineered humanized anti-CD98 mAbs are listed in Table 18.

TABLE 18
Variable region sequences of humanized and further engineered chAb3
and chAb15 clones converted to IgG

| SEQ | | | | |
|---|---|---|---|---|
| ID | | Protein | | |
| NO: | Clone | Region | Residues | V Region |
| 86 | huAb3v1 | VH | | EVQLVESGGGLVQPGRSLRLSCTTSG |
| | | | | FTFIDYYMSWVRQAPGKGLEWLGFIR |
| | | | | NKANRYTTEYSASVKGRFTISRDNSK |
| | | | | SILYLQMNSLKTEDTAVYYCTRDRPA |
| | | | | WFVYWGQGTLVTVSS |
| 16 | huAb3v1 | CDR-H1 | Residues 26-35 | GFTFIDYYMS |
| | | | of SEQ ID NO.: 86 | |
| 87 | huAb3v1 | CDR-H2 | Residues 50-68 | FIRNKANRYTTEYSASVKG |
| | | | of SEQ ID NO.: 86 | |
| 17 | huAb3v1 | CDR-H3 | Residues 101-108 | DRPAWFVY |
| | | | of SEQ ID NO.: 86 | |
| 88 | huAb3v1 | VL | | DIVMTQSPDSLAVSLGERATINCKSS |
| | | | | QSLLYSSNQKNYLAWYQQKPGQSPKL |
| | | | | LIYWASTRESGVPDRFSGSGSGTDFT |
| | | | | LTISSLQAEDVAVYYCQQYYSYPYTF |
| | | | | GGGTKVEIK |
| 13 | huAb3v1 | CDR-L1 | Residues 24-40 | KSSQSLLYSSNQYKNLY |
| | | | of SEQ ID NO.: 88 | |
| 7 | huAb3v1 | CDR-L2 | Residues56-62 | WASTRES |
| | | | of SEQ ID NO.: 88 | |
| 19 | huAb3v1 | CDR-L3 | Residues 95-103 | QQYYSYPYT |
| | | | of SEQ ID NO.: 88 | |
| 89 | huAb3v2 | VH | | EVQLVESGGGINQPGRSLRLSCTTSG |
| | | | | FTFIDYYMSWVRQAPGKGLEWLGFIR |
| | | | | NKAYGYTTEYSASVKGRFTISRDNSK |
| | | | | SILYLQMNSLKTEDTAVYYCTRDRPA |
| | | | | WFVYWGQGTINTVSS |
| 16 | huAb3v2 | CDR-H1 | Residues 26-35 | GFTFIDYYMS |
| | | | of SEQ ID NO.: 89 | |
| 90 | huAb3v2 | CDR-H2 | Residues 50-68 | FIRNKAYGYTTEYSASVKG |
| | | | of SEQ ID NO.: 89 | |
| 17 | huAb3v2 | CDR-H3 | Residues 101-108 | DRPAWFVY |
| | | | of SEQ ID NO.: 89 | |
| 88 | huAb3v2 | VL | | DIVMTQSPDSLAVSLGERATINCKSS |
| | | | | QSLLYSSNQKNYLAWYQQKPGQSPKL |
| | | | | LIYWASTRESGVPDRFSGSGSGIDFT |
| | | | | LTISSLQAEDVAVYYCQQYYSYPYTF |
| | | | | GGGTKVEIK |
| 13 | huAb3v2 | CDR-L1 | Residues 24-40 | KSSQSLLYSSNQYLA |
| | | | of SEQ ID NO.: 88 | |
| 7 | huAb3v2 | CDR-L2 | Residues 56-62 | WASTRES |
| | | | of SEQ ID NO.: 88 | |
| 19 | huAb3v2 | CDR-L3 | Residues 95-103 | QQYYSYPYT |
| | | | of SEQ ID NO.: 88 | |
| 91 | huAb15v1 | VH | | EVQLVESGGGVVQPGRSLRLSCAASG |
| | | | | FTFSDYTMAWVRQAPGKGLEWVATII |
| | | | | YSGRGTYYRDAVKGRFTISRDNSKST |
| | | | | LYLQMNSLRAEDIAVYYCARQSDHTY |
| | | | | YYWGYFDYWGQGTMVTVSS |
| 79 | huAb15v1 | CDR-H1 | Residues 26-35 | GFTFSDYTMA |
| | | | of SEQ ID NO.: 91 | |
| 92 | huAb15v1 | CDR-H2 | Residues 50-66 | TIIYSGRGTYYDAVKG |
| | | | of SEQ ID NO.: 91 | |
| 93 | huAb15v1 | CDR-H3 | Residues 99-112 | QSDHTYYGYFDY |
| | | | of SEQ ID NO.: 91 | |
| 94 | huAb15v1 | VL | | DIVMTQSPDSLAVSLGERATINCKSS |
| | | | | QSLLFSGNQKNYLAWYQQKPGQSPKL |
| | | | | LIYWASTRQSGVPDRFSGSGSGTDFT |
| | | | | LTIRSLQAEDVAIYYCQQYYDVPYTF |
| | | | | GQGTKLEIK |
| 83 | huab15v1 | CDR-L1 | Residues 24-40 | KSSQSLLFSGNQKNYLA |
| | | | of SEQ ID NO.: 94 | |
| 45 | huab15v1 | CDR-L2 | Residues 56-62 | WASTRQS |
| | | | of SEQ ID NO.: 94 | |
| 95 | huab15v1 | CDR-L3 | Residues 95-103 | QQYYDVPYT |
| | | | of SEQ ID NO.: 94 | |
| 96 | huAb15v2 | VH | | EVQLVESGGGVVQPGRSLRLSCAASG |
| | | | | FTFSDYTMAWVRQAPGKGLEWVATII |
| | | | | YSGRGTYYRDAVKGRFTISRDNSKST |
| | | | | LYLQMNSLRAEDTAVYYCARQSDDTY |
| | | | | YYWGYFDYWGQGTMVTVSS |
| 79 | huAb15v21 | CDR-H1 | Residues 26-35 | GFTFSDYTMA |
| | | | of SEQ ID NO.: 96 | |
| 92 | huAb15v2 | CDR-H2 | Residues 50-66 | TIIYSGRGTYYRDAVKG |
| | | | of SEQ ID NO.: 96 | |
| 97 | huAb15v2 | CDR-H3 | Residues 99-112 | QSDDTYYYWGYFDY |
| | | | of SEQ ID NO.: 96 | |
| 94 | huAb15v2 | VL | | DIVMTQSPDSLAVSLGERATINCKSS |
| | | | | QSLLFSGNQKNYLAWYQQKPGQSPKL |
| | | | | LIYWASTRQSGVPDRFSGSGSGTDFT |
| | | | | LTIRSLQAEDVAIYYCQQYYDVPYTF |
| | | | | GQGTKLEIK |
| 83 | huAb15v2 | CDR L2 | Residues 24-40 | KSSQSLLFSGNQKNYLA |
| | | | of SEQ ID NO.: 94 | |
| 45 | huAb15v2 | CDR-L2 | Residues 56-62 | WASTRQS |
| | | | of SEQ ID NO.: 94 | |
| 95 | huAb15v2 | CDR-L3 | Residues 95-103 | QQYYDVPYT |
| | | | of SEQ ID NO.: 94 | |
| 96 | huAb15v3 | VH | | EVQLVESGGGVVQPGRSLRLSCAASG |
| | | | | FTFSDYTMAWVRQAPGKGLEWVATII |
| | | | | YSGRGTYYRDAVKGRFTISRDNSKST |
| | | | | LYLQMNSLRAEDTAVYYCARQSDDTY |
| | | | | YYWGYFDYWGQGTMVTVSS |
| 79 | huAb15v3 | CDR-H1 | Residues 26-35 | GFTFSDYTMA |
| | | | of SEQ ID NO.: 96 | |
| 92 | huAb15v3 | CDR-H2 | Residues 50-66 | TIIYSGRGTYYRDAVKG |
| | | | of SEQ ID NO.: 96 | |
| 97 | huAh15v3 | CDR-H3 | Residues 99-112 | QSDDTYYYWGYFDY |
| | | | of SEQ ID NO.: 96 | |
| 98 | huAb15v3 | VL | | DIVMTQSPDSLAVSLGERATINCKSS |
| | | | | QSLLFSGNQKNYLAWYQQKPGQSPKL |
| | | | | LIYWASTRQSGVPDRESGSGSGTDET |
| | | | | LTIRSLQAEDVAIYYCQQYYGSPYTF |
| | | | | GQGTKLEIK |
| 82 | huAb15v3 | CDR-L1 | Residues 24-40 | KSSQSLLFSGNQKNYLA |
| | | | of SEQ ID NO.: 98 | |
| 45 | huAb15v3 | CDR-L2 | Residues 56-62 | WASTRQS |
| | | | of SEQ ID NO.: 98 | |
| 105 | huAb15v3 | CDR-L3 | Residues 95-103 | QQYYGSPYT |
| | | | of SEQ ID NO.: 98 | |
| 99 | huAb15v4 | VH | | EVQLVESGGGVVQPGRSLRLSCALSG |
| | | | | FTFSDYTMAWVRQAPGKGLEWVATII |
| | | | | YTGRGTYYRDAVKGRFTISRDNSKST |
| | | | | LYLQMNSLRAEDTAVYYCARQSDDTY |
| | | | | YYWGYFDYWGQGTMVTVSS |
| 79 | huAb15v4 | CDR-H1 | Residues 26-35 | GFTFSDYTMA |
| | | | of SEQ ID NO.: 99 | |
| 100 | huAb15v4 | CDR-H2 | Residues 50-66 | TIIYTGRGTYYRDAVKG |
| | | | of SEQ ID NO.: 99 | |
| 97 | huAb15v4 | CDR-H3 | Residues 99-112 | QSDDTYYYWGYFDY |
| | | | of SEQ ID NO.: 99 | |
| 94 | huAb15v4 | VL | | DIVMTQSPDSLAVSLGERATINCKSS |
| | | | | QSLLFSGNQKNYLAWYQQKPGQSPKL |
| | | | | LIYWASTRQSGVPDRFSGSGSGTDET |
| | | | | LTIRSLQAEDVAIYYCQQYYDVPYTF |
| | | | | GQGTKLEIK |
| 83 | huAb15v4 | CDR-L1 | Residues 24-40 | KSSQSLLFSGNQKNYLA |
| | | | of SEQ ID NO.: 94 | |
| 45 | huAb15v4 | CDR-L2 | Residues 56-62 | WASTRQS |
| | | | of SEQ ID NO.: 94 | |
| 95 | huAb15v4 | CDR-L3 | Residues 95-103 | QQYYDVPYT |
| | | | of SEQ ID NO.: 94 | |
| 99 | huAb15v5 | VH | | EVQLVESGGGVVQPGRSLRLSCAASG |
| | | | | FTFSDYTMAWVRQAPGKGLEWVATII |
| | | | | YTGRGTYYRDAVKGRFTISRDNSKST |
| | | | | LYLQMNSLRAEDTAVYYCARQSDDTY |
| | | | | YYWGYFDYWGQGTMVTVSS |
| 79 | huAb15v5 | CDR-H1 | Residues 26-5 | GFTFSDYTMA |
| | | | of SEQ ID NO.: 99 | |
| 100 | huAb15v5 | COR-H2 | Residues 50-66 | TIIYTGRGTYYRDAVKG |
| | | | of SEQ ID NO.: 99 | |
| 97 | huAb15v5 | CDR-H3 | Residues 99-112 | QSDDTYYYWGYFDY |
| | | | of SEQ ID NO.: 99 | |
| 101 | huAb15v5 | VL | | DIVMTQSPDSLAVSLGERATINCKSS |
| | | | | QSLLFSGNQKNYLAWYQQKPGQSPKL |
| | | | | LIYWASTRQSGVPDRFSGSGSGTDFT |
| | | | | LTIRSLQAEDVAIYYCQQYYSSPYTF |
| | | | | GQGTKLEIK |
| 83 | huAb15v5 | CDR-L1 | Residues 24-40 | KSSQSLLFSGNQKNYLA |
| | | | of SEQ ID NO.: 101 | |
| 45 | hukb15v5 | CDR-L2 | Residues 56-62 | WASTRQS |
| | | | of SEQ ID NO.: 101 | |
| 102 | huAb15v5 | CDR-L3 | Residues 95-103 | QQYYSSPYT |
| | | | of SEQ ID NO.: 101 | |
| 103 | huAb15v6 | VH | | EVQLVESGGGVVQPGRSLRLSCAASG |
| | | | | FTFSDYTMAWVRQAPGKGLEWVATII |
| | | | | YDARGTYYRDAVKGRFTISRDNSKST |
| | | | | LYLQMNSLRAEDTAVYYCARQSDDTY |
| | | | | YYWGYFDYWGQGTMVTVSS |
| 79 | hukb15v6 | CDR-H1 | Residues 26-35 | GFTFSDYTMA |
| | | | of SEQ ID NO.: 103 | |
| 104 | huAb15v6 | CDR-H2 | Residues 50-66 | TIIYDARGTYYRDAVKG |
| | | | of SEQ ID NO.: 103 | |
| 97 | huAb15v6 | CDR-H3 | Residues 99-112 | QSDDTYYYWGYFDY |
| | | | of SEQ ID NO.: 103 | |
| 101 | huAb15v6 | VL | | DIVMTQSPDSLAVSLGERATINCKSS |
| | | | | QSLLFSGNQKNYLAWYQQKPGQSPKL |
| | | | | LIYWASTRQSGVPDRFSGSGSGTDFT |
| | | | | LTIRSLQAEDVAITYCQQYYSSPYTF |
| | | | | GQGTKLEIK |
| 83 | huAb15v6 | CDR-L1 | Residues 24-40 | KSSQSLLFSGNQKNYLA |
| | | | of SEQ ID NO.: 101 | |
| 45 | huAb15v6 | CDR-L2 | Residues 56-62 | WASTRQS |
| | | | of SEQ ID NO. | |
| 102 | huAb15v6 | CDR-L3 | Residues 95-103 | QQYYSSPYT |
| | | | of SEQ ID NO.: 101 | |
| 103 | huAb15v7 | VH | | EVQLVESGGGVVQPGRSLRLSCAASG |
| | | | | FTFSDYTMAWVRQAPGKGLEWVATII |
| | | | | YDARGTYYRDAVKGRFTISRDNSKST |
| | | | | LYLQMNSLRAEDTAVYYCARQSDDTY |
| | | | | YYWGYFDYWGQGTMVTVSS |
| 79 | huAb15v7 | CDR-H1 | Residues 26-35 | GFTFSDYTMA |
| | | | of SEQ ID NO.: 103 | |
| 104 | huAb15v7 | CDR-H2 | Residues 50-66 | TIIYDARGTYYDAVKG |
| | | | of SEQ ID NO.: 103 | |
| 97 | huAb15v7 | CDR-H3 | Residues 99-112 | QSDDTYYYNGYFDY |
| | | | of SEQ ID NO.: 103 | |
| 98 | huAb15v7 | VL | | DIVMTQSPDSLAVSLGERATINCKSS |
| | | | | QSLLFSGNQKNYLAWYQQKPGQSPKL |
| | | | | LIYWASTRQSGVPDRFSGSGSGTDFT |
| | | | | LTIRSLQAEDVAIYYCQQYYGSPYTF |
| | | | | GQGTKLEIK |
| 83 | huAb15v7 | CDR-L1 | Residues 24-40 | KSSQSLLFSGNQYLA |
| | | | of SEQ ID NO.: 98 | |
| 45 | huAb15v7 | CDR-L2 | Residues 56-62 | WASTRQS |
| | | | of SEQ ID NO.: 98 | |
| 105 | huAb15v7 | CDR-L3 | Residues 95-103 | QQYYGSPYT |
| | | | of SEQ ID NO.: 98 | |

The binding kinetics of the recombinant anti-CD98 chimeric antibodies for purified recombinant CD98 protein (extracellular domain, ECD) were determined by surface plasmon resonance-based measurements, as described in the Example 4. Results are shown in Table 19.

TABLE 19
Biacore Kinetics of Anti-CD98 Humanized Antibodies
Binding to Human and Cynomolgus Monkey CD98.

| | Kinetics on Biacore | | | | | |
|---|---|---|---|---|---|---|
| | huCD98 ECD | | | cyCD98 ECD | | |
| Humanized | kₐ | k_{d} | K_{D} | kₐ | k_{d} | K_{D} |
| Clone | (M⁻¹s⁻¹) | (s⁻¹) | (M) | (M⁻¹s⁻¹) | (s⁻¹) | (M) |
| huAb3v1 | 1.8E+05 | 2.3E−03 | 1.3E−08 | 4.0E+04 | 9.2E−04 | 2.3E−08 |
| huAb3v2 | 2.6E+05 | 1.3E−03 | 4.9E−09 | 2.9E+04 | 4.9E−04 | 1.7E−08 |
| huAb15v1 | 5.3E+04 | 2.3E−05 | 4.3E−10 | 2.6E+04 | 2.6E−05 | 1.0E−09 |
| huAb15v2 | 4.2E+04 | 4.6E−05 | 1.1E−09 | 2.1E+04 | 4.8E−05 | 2.3E−09 |
| huAb15v3 | 4.4E+04 | 8.5E−05 | 2.0E−09 | 1.8E+04 | 1.4E−04 | 7.6E−09 |
| huAb15v4 | 3.5E+04 | 2.8E−05 | 8.1E−10 | 2.0E+04 | 5.0E−05 | 2.6E−09 |
| huAb15v5 | 4.1E+04 | 1.3E−04 | 3.1E−09 | 1.7E+04 | 1.8E−04 | 1.1E−08 |
| huAb15v6 | 3.8E+04 | 2.6E−04 | 6.7E−09 | 1.4E+04 | 4.9E−04 | 3.4E−08 |
| huAb15v7 | 3.5E+04 | 2.1E−04 | 5.9E−09 | 1.4E+04 | 4.3E−04 | 3.0E−08 |

hu = human;
cy = cynomolgus monkey;
ECD = extracellular domain;
E + Y = ×10^{Y};
E − Y = ×10^{-Y}

### Example 13. Bcl-xL Inhibitor Conjugation with Humanized Anti-CD98 mAbs

The above nine humanized anti-CD98 mAbs were tested for conjugation with the Bcl-xL inhibitor synthon CZ according to Method A, as described in Example 5. Precipitation was observed for nine anti-CD98 mAbs as set out in Table 20.

TABLE 20
Humanized Anti-CD98 mAbs Conjugated
with Bcl-xL Inhibitor CZ Payload

| Human- | DAR | |
|---|---|---|
| ized | by | Key observation for ADC solution during and after |
| clone | MS | conjugation |
| huAb3v1 | 3.4 | Cloudy during conjugation; precipitated at 4° C. |
| | | storage |
| huAb3v2 | 3.0 | Precipitated during and after conjugation; aggregation |
| | | level 16.52% |
| huAb15v1 | 1.0 | Precipitated during conjugation; inefficient conjugation |
| huAb15v2 | 2.1 | Precipitated during conjugation; inefficient conjugation |
| huAb15v3 | 1.2 | Precipitated during conjugation; inefficient conjugation |
| huAb15v4 | 1.9 | Precipitated during conjugation; inefficient conjugation |
| huAb15v5 | 1.0 | Precipitated during conjugation; inefficient conjugation |
| huAb15v6 | 2.0 | Precipitated during conjugation; inefficient conjugation |
| huAb15v7 | 2.2 | Precipitated during conjugation; inefficient conjugation |

### Example 14. Antibody Framework Re-Engineering of Humanized anti-CD9 mAbs to Improve Conjugation Efficiency with Bcl-L Inhibitor

In order to evaluate whether different antibody framework could impact the conjugation properties of the anti-CD98 mAbs to Bcl-xL inhibitor on synthons different iteration of humanized variants for chAb3 and chAb15 using alternative frameworks compared to antibodies listed in Table 14 and 15, were expressed as full-length IgGH and evaluated for human CD98 binding. Humanized framework engineered antibodies that maintained binding to human CD98 are listed in Table 21.

TABLE 21
Framework Engineering of Humanized Anti-CD98 mAbs

| Re-engineered | Parental | | |
|---|---|---|---|
| Humanized | humanized | VH | VL |
| clone | clone | framework | framework |
| huAb101 | huAb3v1 | IGHV3-15 | IGKV2-40 |
| huAb102 | huAb3v1 | IGHV3-72 | IGKV2-40 |
| huAb103 | huAb3v2 | IGHV3-15 | IGKV2-40 |
| huAb104 | huAb3v2 | IGHV3-72 | IGKV2-40 |
| huAb105 | huAb15v1 | IGHV3-7 | IGKV2-40 |
| huAb106 | huAb15v1 | IGHV1-46 | IGKV2-40 |
| huAb107 | huAb15v2 | IGHV3-7 | IGKV2-40 |
| huAb108 | huAb15v2 | IGHV1-46 | IGKV2-40 |
| huAb109 | huAb15v6 | IGHV3-7 | IGKV2-40 |
| huAb110 | huAb15v6 | IGHV1-46 | IGKV2-40 |

The VH and VL sequences of these re-engineered anti-CD98 mAbs are listed in Table 22.

TABLE 22
Variable region sequences of humanized and framework engineered
chAb3 and chAb15 clones converted to IgG

| SEQ | | | | |
|---|---|---|---|---|
| ID | | Protein | | |
| NO: | Clone | Regioin | Residues | V Region |
| 106 | huAb101 | VH | | EVQLVESGGGLVKPGGSLRLSCATSGFT |
| | | | | FIDYYMSWVRQAPGKGLEWLGFIRNKAN |
| | | | | RYTTEYSASVKGRFTQSRDNSKSILYLQ |
| | | | | MNSLKTEDTAVYYCTRDRPAWFVYWGQG |
| | | | | TLVTVSS |
| 16 | huAb101 | CDR-H1 | Residues 26-35 | GFTFIDYYMS |
| | | | of SEQ ID NO.: 106 | |
| 87 | huAb101 | CDR-H2 | Residues 50-68 | FIRNKANRYTTEYSASVKG |
| | | | of SEQ ID NO.: 106 | |
| 17 | huAb101 | CDR-H3 | Residues 101-108 | DRPAWFVY |
| | | | of SEQ ID NO.: 106 | |
| 107 | huAb101 | VL | | DIVMTQTPLSLPVTPGEPASISCKSSQ |
| | | | | SLLYSSNQKNYLAWYLQKPGQSPQLLI |
| | | | | YWASTRESGVPDRFSGSGSGTDFTLKI |
| | | | | SRVEAEDVGVYYCQQYYSYPYTFGGGT |
| | | | | KVEIK |
| 13 | huAb101 | CDR-L1 | Residues 24-40 | KSSQSLLYSSNQKNYLA |
| | | | of SEQ ID NO.: 107 | |
| 7 | huAb101 | CDR-L2 | Residues 56-62 | WASTRES |
| | | | of SEQ ID NO.: 107 | |
| 19 | huAb101 | CDR-L3 | Residues 95-103 | QQYYSYPYT |
| | | | of SEQ ID NO.: 107 | |
| 108 | huAb102 | VH | | EVQLVESGGGLVQPGGSLRLSCATSGF |
| | | | | TFIDYYMSWVRQAPGKGLEWLGFIRNK |
| | | | | ANRYTTEYSASVKGRFTISRDNSKSIL |
| | | | | YLQMNSLKTEDTAVYYCTRDRPAWFVY |
| | | | | WGQGTLVTVSS |
| 16 | huAb102 | CDR-H1 | Residues 26-35 | GFTFIDYYMS |
| | | | of SEQ ID NO.: 108 | |
| 87 | huAb102 | CDR-H2 | Residues 50-68 | FIRNKANRYTTEYSASVKG |
| | | | of SEQ ID NO.: 108 | |
| 17 | huAb102 | CDR-H3 | Residues 101-108 | DRPAWFVY |
| | | | of SEQ ID NO.: 108 | |
| 107 | huAb102 | VL | | DIVMTQTPLSLPVTPGEPASISCKSSQ |
| | | | | SLLYSSNQKNYLAWYLQKPGQSPQLLI |
| | | | | YWASTRESGVPDRFSGSGSGTDFTLKI |
| | | | | SRVEAEDVGVYYCQQYYSYPYTFGGGT |
| | | | | KVEIK |
| 13 | huAb102 | CDR-L1 | Residues 24-40 | KSSQSLLYSSNQKNYLA |
| | | | of SEQ ID NO.: 107 | |
| 7 | huAb102 | CDR-L2 | Residues 56-62 | WASTRES |
| | | | of SEQ ID NO.: 107 | |
| 19 | huAb102 | CDR-L3 | Residues 95-103 | QQYYSYPYT |
| | | | of SEQ ID NO.: 107 | |
| 109 | huAb103 | VH | | EVQLVESGGGLVQPGGSLRLSCATSGF |
| | | | | TFIDYYMSWVRQAPGKGLEWLGFIRNK |
| | | | | ANRYTTEYSASVKGRFTISRDNSKSIL |
| | | | | YLQMNSLKTEDTAVYYCTRDRPAWFVY |
| | | | | WGQGTLVTVSS |
| 16 | huAb103 | CDR-H1 | Residues 26-35 | GFTFIDYYMS |
| | | | of SEQ ID NO.: 109 | |
| 90 | huAb103 | CDR-H2 | Residues 50-68 | FIRNKAYGYTTEYSASVKG |
| | | | of SEQ ID NO.: 109 | |
| 17 | huAb103 | CDR-H3 | Residues 101-108 | DRPAWFVY |
| | | | of SEQ ID NO.: 109 | |
| 107 | huAb103 | VL | | DIVMTQTPLSLPVTPGEPASISCKSSQ |
| | | | | SLLYSSNQKNYLAWYLQKPGQSPQLLI |
| | | | | YWASTRESGVPDRFSGSGSGTDFTLKI |
| | | | | SRVEAEDVGVYYCQQYYSYPYTFGGGT |
| | | | | KVEIK |
| 13 | huAb103 | CDR-L1 | Residues 24-40 | KSSQSLLYSSNQKNYLA |
| | | | of SEQ ID NO.: 107 | |
| 7 | huAb103 | CDR-L2 | Residues 56-62 | WASTRES |
| | | | of SEQ ID NO.: 107 | |
| 19 | huAb103 | CDR-L3 | Residues 95-103 | QQYYSYPYT |
| | | | of SEQ ID NO.: 107 | |
| 110 | huAb104 | VH | | EVQLVESGGGLVQPGGSLRLSCATSGF |
| | | | | TFIDYYMSWVRQAPGKGLEWLGFIRNK |
| | | | | AYGYTTEYSASVKGRFTISRDNSKSIL |
| | | | | YLQMNSLKTEDTAVYYCTRDRPAWFVY |
| | | | | WGQGTLVTVSS |
| 16 | huAb104 | CDR-H1 | Residues 26-35 | GFTFIDYYMS |
| | | | of SEQ ID NO.: 110 | |
| 90 | huAb104 | CDR-H2 | Residues 50-68 | FIRNKAYGYTTEYSASVKG |
| | | | of SEQ ID NO.: 110 | |
| 17 | huAb104 | CDR-H3 | Residues 101-108 | DRPAWFVY |
| | | | of SEQ ID NO.: 110 | |
| 107 | huAb104 | VL | | DIVMTQTPLSLPVTPGEPASISCKSSQ |
| | | | | SLLYSSNQKNYLAWYLQKPGQSPQLLI |
| | | | | YWASTRESGVPDRFSGSGSGTDFTLKI |
| | | | | SRVEAEDVGVYYCQQYYSYPYTFGGGT |
| | | | | KVEIK |
| 13 | huAb104 | CDR-L1 | Residues 24-40 | KSSQSLLYSSNQKNYLA |
| | | | of SEQ ID NO.: 107 | |
| 7 | huAb104 | CDR-L2 | Residues 56-62 | WASTRES |
| | | | of SEQ ID NO.: 107 | |
| 19 | huAb104 | CDR-L3 | Residues 95-103 | QQYYSYPYT |
| | | | of SEQ ID NO.: 107 | |
| 111 | huAb105 | VH | | EVQLVESGGGLVQPGGSLRLSCAASGF |
| | | | | TFSDYTMSWVRQAPGKGLEWVATIIYS |
| | | | | GRGTYYRDAVKGRFTISRDNAKNTLYL |
| | | | | QMNSLKTEDTAVYYCARQSDHTYYYWG |
| | | | | YFDYWGQGTMVTVSS |
| 79 | huAb105 | CDR-H1 | Residues 26-35 | GFTFSDYTMA |
| | | | of SEQ ID NO.: 111 | |
| 92 | huAb105 | CDR-H2 | Residues 50-66 | TIIYSGRGTYYRDAVKG |
| | | | of SEQ ID NO.: 111 | |
| 93 | huAb105 | CDR-H3 | Residues 99-112 | QSDHTYYYWGYFDY |
| | | | of SEQ ID NO.: 111 | |
| 112 | huAb105 | VL | | DIVMTQTPLSLPVTPGEPASISCKSSQ |
| | | | | SLLYSSNQKNYLAWYLQKPGQSPQLLI |
| | | | | YWASTRQSGVPDRFSGSGSGTDFTLKI |
| | | | | SRVEAEDVGVYYCQQYYSYPYTFGQGT |
| | | | | KVEIK |
| 83 | huAb105 | CDR-L1 | Residues 24-40 | KSSQSLLFSGNQKNYLA |
| | | | of SEQ ID NO.: 112 | |
| 45 | huAb105 | CDR-L2 | Residues 56-62 | WASTRQS |
| | | | of SEQ ID NO.: 112 | |
| 95 | huAb105 | CDR-L3 | Residues 95-103 | QQYYDVPYT |
| | | | of SEQ ID NO.: 112 | |
| 113 | huAb106 | VH | | EVQLVESGGGLVQPGGSLRLSCAASGF |
| | | | | TFSDYTMAWVRQAPGKGLEWVATIIYS |
| | | | | GRGTYYRDAVKGRFTISRDNAKNTLYL |
| | | | | ELSSLRSEDTAVYYCARQSDHTYYYWG |
| | | | | YFDYWGQGTMVTVSS |
| 79 | huAb106 | CDR-H1 | Residues 26-35 | GFTFSDYTMA |
| | | | of SEQ ID NO.: 113 | |
| 92 | huAb106 | CDR-H2 | Residues 50-66 | TIIYSGRGTYYRDAVKG |
| | | | of SEQ ID NO.: 113 | |
| 93 | huAb106 | CDR-H3 | Residues 99-112 | QSDHTYYYWGYFDY |
| | | | of SEQ ID NO.: 113 | |
| 112 | huAb106 | VL | | DIVMTQTPLSLPVTPGEPASISCKSSQ |
| | | | | SLLYSSNQKNYLAWYLQKPGQSPQLLI |
| | | | | YWASTRQSGVPDRFSGSGSGTDFTLKI |
| | | | | SRVEAEDVGVYYCQQYYSYPYTFGQGT |
| | | | | KVEIK |
| 83 | huAb106 | CDR-L1 | Residues 24-40 | KSSQSLLFSGNQKNYLA |
| | | | of SEQ ID NO.: 112 | |
| 45 | huAb106 | CDR-L2 | Residues 56-62 | WASTRQS |
| | | | of SEQ ID NO.: 112 | |
| 95 | huAb106 | CDR-L3 | Residues 95-103 | QQYYDVPYT |
| | | | of SEQ ID NO.: 112 | |
| 114 | huAb107 | VH | | EVQLVESGGGLVQPGGSLRLSCAASGF |
| | | | | TFSDYTMSWVRQAPGKGLEWVATIIYS |
| | | | | GRGTYYRDAVKGRFTISRDNAKNTLYL |
| | | | | QMNSLKTEDTAVYYCARQSDHTYYYWG |
| | | | | YFDYWGQGTMVTVSS |
| 79 | huAb107 | CDR-H1 | Residues 26-35 | GFTFSDYTMA |
| | | | of SEQ ID NO.: 114 | |
| 92 | huAb107 | CDR-H2 | Residues 50-66 | TIIYSGRGTYYRDAVKG |
| | | | of SEQ ID NO.: 114 | |
| 97 | huAb107 | CDR-H3 | Residues 99-112 | QSDHTYYYWGYFDY |
| | | | of SEQ ID NO.: 114 | DIVMTQTPLSLPVTPGEPASISCKSSQ |
| 112 | huAb107 | VL | | SLLFSGNQKNYLAWYLQKPGQSPQLLI |
| | | | | YWASTRQSGVPDRFSGSGSGTDFTLKI |
| | | | | SRVEAEDVGVYYCQQYYSYPYTFGQGT |
| | | | | KVEIK |
| 83 | huAb106 | CDR-L1 | Residues 24-40 | KSSQSLLFSGNQKNYLA |
| | | | of SEQ ID NO.: 112 | |
| 45 | huAb106 | CDR-L2 | Residues 56-62 | WASTRQS |
| | | | of SEQ ID NO.: 112 | |
| 95 | huAb106 | CDR-L3 | Residues 95-103 | QQYYDVPYT |
| | | | of SEQ ID NO.: 112 | |
| 115 | huAb108 | VH | | EVQLVESGGGLVQPGGSLRLSCAASGF |
| | | | | TFSDYTMSWVRQAPGKGLEWVATIIYS |
| | | | | GRGTYYRDAVKGRFTISRDNAKNTLYL |
| | | | | QMNSLKTEDTAVYYCARQSDHTYYYWG |
| | | | | YFDYWGQGTMVTVSS |
| 79 | huAb108 | CDR-H1 | Residues 26-35 | GFTFSDYTMA |
| | | | of SEQ ID NO.: 115 | |
| 92 | huAb108 | CDR-H2 | Residues 50-66 | TIIYSGRGTYYRDAVKG |
| | | | of SEQ ID NO.: 115 | |
| 97 | huAb108 | CDR-H3 | Residues 99-112 | QSDDTYYYWGYFDY |
| | | | of SEQ ID NO.: 115 | |
| 112 | huAb108 | VL | | DIVMTQTPLSLPVTPGEPASISCKSSQ |
| | | | | SLLFSGNQKNYLAWYLQKPGQSPQLLI |
| | | | | YWASTRQSGVPDRFSGSGSGTDFTLKI |
| | | | | SRVEAEDVGVYYCQQYYSYPYTFGQGT |
| | | | | KVEIK |
| 83 | huAb108 | CDR-L1 | Residues 24-40 | KSSQSLLFSGNQKNYLA |
| | | | of SEQ ID NO.: 112 | |
| 45 | huAb108 | CDR-L2 | Residues 56-62 | WASTRQS |
| | | | of SEQ ID NO.: 112 | |
| 95 | huAb108 | CDR-L3 | Residues 95-103 | QQYYDVPYT |
| | | | of SEQ ID NO.: 112 | |
| 116 | huAb109 | VH | | EVQLVESGGGLVQPGGSLRLSCAASGF |
| | | | | TFSDYTMAWVRQAPGKGLEWVATIIYD |
| | | | | ARGTYYRDAVKGRFTISRDNAKNTLYL |
| | | | | QMNSLKTEDTAVYYCARQSDHTYYYWG |
| | | | | YFDYWGQGTMVTVSS |
| 79 | huAb109 | CDR-H1 | Residues 26-35 | GFTFSDYTMA |
| | | | of SEQ ID NO.: 116 | |
| 104 | huAb109 | CDR-H2 | Residues 50-66 | TIIYSGRGTYYRDAVKG |
| | | | of SEQ ID NO.: 116 | |
| 97 | huAb109 | CDR-H3 | Residues 99-112 | QSDDTYYYWGYFDY |
| | | | of SEQ ID NO.: 116 | |
| 117 | huAb109 | VL | | DIVMTQTPLSLPVTPGEPASISCKSSQ |
| | | | | SLLFSGNQKNYLAWYLQKPGQSPQLLI |
| | | | | YWASTRQSGVPDRFSGSGSGTDFTLKI |
| | | | | SRVEAEDVGVYYCQQYYSYPYTFGQGT |
| | | | | KVEIK |
| 83 | huAb109 | CDR-L1 | Residues 24-40 | KSSQSLLFSGNQKNYLA |
| | | | of SEQ ID NO.: 117 | |
| 45 | huAb109 | CDR-L2 | Residues 56-62 | WASTRQS |
| | | | of SEQ ID NO.: 117 | |
| 102 | huAb109 | CDR-L3 | Residues 95-103 | QQYYDVPYT |
| | | | of SEQ ID NO.: 117 | |
| 118 | huAb110 | VH | | EVQLVESGGGLVQPGGSLRLSCAASGF |
| | | | | TFSDYTMAWVRQAPGKGLEWVATIIYD |
| | | | | ARGTYYRDAVKGRFTISRDNAKNTLYL |
| | | | | ELSSLRSEDTAVYYCARQSDDTYYYWG |
| | | | | YFDYWGQGTMVTVSS |
| 79 | huAb110 | CDR-H1 | Residues 26-35 | GFTFSDYTMA |
| | | | of SEQ ID NO.: 118 | |
| 104 | huAb110 | CDR-H2 | Residues 50-66 | TIIYSGRGTYYRDAVKG |
| | | | of SEQ ID NO.: 118 | |
| 97 | huAb110 | CDR-H3 | Residues 99-112 | QSDDTYYYWGYFDY |
| | | | of SEQ ID NO.: 118 | |
| 117 | huAb110 | VL | | DIVMTQTPLSLPVTPGEPASISCKSSQ |
| | | | | SLLFSGNQKNYLAWYLQKPGQSPQLLI |
| | | | | YWASTRQSGVPDRFSGSGSGTDFTLKI |
| | | | | SRVEAEDVGVYYCQQYYSYPYTFGQGT |
| | | | | KVEIK |
| 83 | huAb110 | CDR-L1 | Residues 24-40 | KSSQSLLFSGNQKNYLA |
| | | | of SEQ ID NO.: 117 | |
| 45 | huAb110 | CDR-L2 | Residues 56-62 | WASTRQS |
| | | | of SEQ ID NO.: 117 | |
| 102 | huAb101 | CDR-L3 | Residues 95-103 | QQYYSSPYT |
| | | | of SEQ ID NO.: 117 | |
| 119 | huAb106v1 | VH | | EVQLVESGGGLVQPGGSLRLSCAASGF |
| | | | | TFSDYTMAWVRQAPGKGLEWVATIIYD |
| | | | | GRGTYYRDAVKGRFTISRDNAKNTLYL |
| | | | | ELSSLRSEDTAVYYCARQSDHTYYYWG |
| | | | | YFDYWGQGTMVTVSS |
| 79 | huAb1106v1 | CDR-H1 | Residues 26-35 | GFTFSDYTMA |
| | | | of SEQ ID NO.: 119 | |
| 92 | huAb106v1 | CDR-H2 | Residues 50-66 | TIIYSGRGTYYRDAVKG |
| | | | of SEQ ID NO.: 119 | |
| 93 | huAb106v1 | CDR-H3 | Residues 99-112 | QSDHTYYYWGYFDY |
| | | | of SEQ ID NO.: 119 | |
| 112 | huAb106v1 | VL | | DIVMTQTPLSLPVTPGEPASINCKSSQ |
| | | | | SLLFSGNQKNYLAWYLQKPGQSPQLLI |
| | | | | YWASTRQSGVPDRFSGSGSGTDFTLKI |
| | | | | SRVEAEDVGVYYCQQYYDVPYTFGQGT |
| | | | | KVEIK |
| 83 | huAb106v1 | CDR-L1 | Residues 24-40 | KSSQSLLFSGNQKNYLA |
| | | | of SEQ ID NO.: 112 | |
| 45 | huAb106v1 | CDR-L2 | Residues 56-62 | WASTRQS |
| | | | of SEQ ID NO.: 112 | |
| 95 | huAb106v1 | CDR-L3 | Residues 95-103 | QQYYDVPYT |
| | | | of SEQ ID NO.: 112 | |
| 120 | huAb108v1 | VH | | EVQLVQSGGGLVQPGGSLRLSCKASGF |
| | | | | TFSDYTMAWVRQAPGKGLEWVATIIYS |
| | | | | GRGTYYRDAVKGRFTISRDNAKNTLYL |
| | | | | ELSSLRSEDTAVYYCARQSDDTYYYWG |
| | | | | YFDYWGQGTMVTVSS |
| 79 | huAb108v1 | CDR-H1 | Residues 26-35 | GFTFSDYTMA |
| | | | of SEQ ID NO.: 120 | |
| 92 | huAb108v1 | CDR-H2 | Residues 50-66 | TIIYSGRGTYYRDAVKG |
| | | | of SEQ ID NO.: 120 | |
| 97 | huAb108v1 | CDR-H3 | Residues 99-112 | QSDDTYYYWGYFDY |
| | | | of SEQ ID NO.: 120 | |
| 112 | huAb108v1 | VL | | DIVMTQTPLSLPVTPGEPASINCKSSQ |
| | | | | SLLFSGNQKNYLAWYLQKPGQSPQLLI |
| | | | | YWASTRQSGVPDRFSGSGSGTDFTLKI |
| | | | | SRVEAEDVGVYYCQQYYSYPYTFGQGT |
| | | | | KVEIK |
| 83 | huAb108v1 | CDR-L1 | Residues 24-40 | KSSQSLLFSGNQKNYLA |
| | | | of SEQ ID NO.: 120 | |
| 45 | huAb108v1 | CDR-L2 | Residues 56-62 | WASTRQS |
| | | | of SEQ ID NO.: 120 | |
| 95 | huAb108v1 | CDR-L3 | Residues 95-103 | QQYYDVPYT |
| | | | of SEQ ID NO.: 120 | |
| 121 | huAb110v1 | VH | | EVQLVESGGGLVQPGGSLRLSCKASGF |
| | | | | TFSDYTMAWVRQAPGKGLEWVATIIYS |
| | | | | ARGTYYRDAVKGRFTISRDNAKNTLYL |
| | | | | ELSSLRSEDTAVYYCARQSDDTYYYWG |
| | | | | YFDYWGQGTMVTVSS |
| 79 | huAb110v1 | CDR-H1 | Residues 26-35 | GFTFSDYTMA |
| | | | of SEQ ID NO.: 121 | |
| 104 | huAb110v1 | CDR-H2 | Residues 50-66 | TIIYDSRGTYYRDAVKG |
| | | | of SEQ ID NO.: 121 | |
| 97 | huAb110v1 | CDR-H3 | Residues 99-112 | QSDDTYYYWGYFDY |
| | | | of SEQ ID NO.: 121 | |
| 117 | huAb110v1 | VL | | DIVMTQTPLSLPVTPGEPASISCKSSQ |
| | | | | SLLFSGNQKNYLAWYLQKPGQSPQLLI |
| | | | | YWASTRQSGVPDRFSGSGSGTDFTLKI |
| | | | | SRVEAEDVGVYYCQQYYSSPYTFGQGT |
| | | | | KVEIK |
| 83 | huAb110v1 | CDR-L1 | Residues 24-40 | KSSQSLLFSGNQKNYLA |
| | | | of SEQ ID NO.: 117 | |
| 45 | huAb110v1 | CDR-L2 | Residues 56-62 | WASTRQS |
| | | | of SEQ ID NO.: 117 | |
| 102 | huAb110v1 | CDR-L3 | Residues 95-103 | QQYYSYPYT |
| | | | of SEQ ID NO.: 117 | |

TABLE 23
Heavy Chain and Light Chain sequences of Humanized
anti-CD98 Antibodies

| | | HC | | LC |
|---|---|---|---|---|
| | | SEQ | | SEQ |
| | | ID | | ID |
| Ab | Heavy Chain Sequence | NO: | Light Chain Sequence | NO: |
| huAb102 | EVQLVESGGGLVQPGGSLRLSCAT | 158 | DIVMTQTPLSLPVTPGEPASISCKSS | 159 |
| | SGFTFIDYYMSWVRQAPGKGLEWL | | QSLLYSSNQKNYLAWYLQKPGQSPQL | |
| | GFIRNEANRYTTEYSASVKGRFTI | | LIYWASTRESGVPDRFSGSGSGTDFT | |
| | SRDNSKSILYLQMNSLKTEDTAVY | | LKISRVEAEDVGVYYCQQYYSYPYTF | |
| | YCTRDRPAWFVYWGQGTLVTVSSA | | GGGTKVEIKRTVAAPSVFIFPPSDEQ | |
| | STKGPSVFPLAPSSKSTSGGTAAL | | LKSGTASVVCLLNNFYPREAKVQWKV | |
| | GCLVKDYFPEPVTVSWNSGALTSG | | DNALQSGNSQESVTEQDSKDSTYS | |
| | VHTFPAVLQSSGLYSLSSVVTVPS | | LSSTLTLSKADYEKHKVYACEVTHQG | |
| | SSLGTQTYICNVNHKPSNTKVDKN | | LSSPVTKSFNRGEC | |
| | VEPKSCDKTHTCPPCPAPEAAGGP | | | |
| | SVFLFPPKPKDTLMISRTPEVTCV | | | |
| | VVDVSHEDPEVKFNMYVDGVEVHN | | | |
| | AKTKPREEQYNSTYRVVSVLTVLH | | | |
| | QDWLNGKEYKCKVSNKAIPAPIEK | | | |
| | TISKAKGQPREPQVYTLPPSREEM | | | |
| | TKNQVSLTCLVKGFYPSDIAVEWE | | | |
| | SNGQPENNYKTTPPVLDSDGSFFL | | | |
| | YSKLTVDKSRWQQGNVFSCSVMHE | | | |
| | ALHNHYTQKSLSLSPGK | | | |
| huAb104 | EVQLVESGGGLVQPGGSLRLSCAT | 160 | DIVMTQTPLSLPVTPGEPASISCKSS | 161 |
| | SGFTFIDYYMSWVRQAPGKGLEWL | | QSLLYSSNQKNYLAWYLQKPGQSPQL | |
| | GFIRNKAYGYTTEYSASVKGRFTI | | LIYWASTRESGVPDRFSGSGSGTDFT | |
| | SRDNSKSILYLQMNSLKTEDTAVY | | LKISRVEAEDVGVYYCQQYYSYPYTF | |
| | YCTRDRPAWFVYWGQGTLVTVSSA | | GGGTKVEIKRTVAAPSVFIFPPSDEQ | |
| | STKGPSVEPLAPSSKSTSGGTAAL | | LKSGTASVVCLLNNFYPREAKVQWKV | |
| | GCLVKDYFPEPVTVSWNSGALTSG | | DNALQSGNSQESVTEQDSKDSTYS | |
| | VHTFPAVLQSSGLYSLSSVVTVPS | | LSSTLTLSKADYEKHKVYACEVTHQG | |
| | SSLGTQTYICNVNHKPSNTKVDKK | | LSSPVTKSFNRGEC | |
| | VEPKSCDKTHTCPPCPAPEAAGGP | | | |
| | SVFLFPPKPKDTLMISRTPEVTCV | | | |
| | VVDVSHEDPEVKFNWYVDGVEVHN | | | |
| | AKTKPREEQYNSTYRVVSVLTVLH | | | |
| | QDWLNGKEYKCKVSNKALPAPIEK | | | |
| | TISKAKGQPREPQVYTLPPSREEM | | | |
| | TKNQVSLTCLVKGFYPSDIAVEWE | | | |
| | SNGQPENNYKTTPPVLDSDGSFFL | | | |
| | YSKLTVDKSRWQQGNVFSCSVMHE | | | |
| | ALHNHYTQKSLSLSPGK | | | |
| huAb108 | EVQLVQSGAEVKKPGASVKVSCKA | 162 | DIVMTQTPLSLPVTPGEPASINCKSS | 163 |
| | SGFTFSDYTMAWVRQAPGQGLEWV | | QSLLFSGNQKNYLAWYLQKPGQSPQL | |
| | ATIIYSGRGTYYRDAVKGRFTITR | | LIYWASTRQSGVPDRFSGSGSGTDFT | |
| | DNSTSTLYLELSSLRSEDTAVYYC | | LKISRVEAEDVGIYYCQQYYDVPYTF | |
| | ARQSDDTYYYWGYFDYWGQGTMVT | | GQGTKLEIKRTVAAPSVFIFPPSDEQ | |
| | VSSASTKGPSVFPLAPSSKSTSGG | | LKSGTASVVCLLNNFYPREAKVQWKV | |
| | TAALGCLVKDYFPEPVTVSWNSGA | | DNALQSGNSQESVTEQDSKDSTYS | |
| | LTSGVHTFPAVLQSSGLYSLSSVV | | LSSTLTLSKADYEKHKVYACEVTHQG | |
| | TVPSSSLGTQTYICNVNHKPSNTK | | LSSPVTKSFNRGEC | |
| | VDKKVEPKSCDKTHTCPPCPAPEA | | | |
| | AGGPSVFLFPPKPKDTLMISRTPE | | | |
| | VTCVVVDVSHEDPEVKFNWYVDGV | | | |
| | EVHNAKTKPREEQYNSTYRVVSVL | | | |
| | TVLHQDWLNGKEYKCKVSNKALPA | | | |
| | PIEKTISKAKGQPREPQVYTLPPS | | | |
| | REEMTKNQVSLTCLVKGFYPSDIA | | | |
| | VEWESNGQPENNYKTTPPVLDSDG | | | |
| | SFFLYSKLTVDKSRWQQGNVFSCS | | | |
| | VMHEALHNHYTQKSLSLSPGK | | | |
| huAb110 | EVQLVQSGAEVKKPGASVKVSCKA | 164 | DIVMTQTPLSLPVTPGEPASINCKSS | 165 |
| | SGFTFSDYTMAWVRQAPGQGLEWV | | QSLLFSGNQKNYLAWYLQKPGQSPQL | |
| | ATIIYDARGTYYRDAVKGRFTITR | | LIYWASTRQSGVPDRFSGSGSGTDFT | |
| | DNSTSTLYLELSSLRSEDTAVYYC | | LKISRVEAEDVGIYYCQQYYSSPYTF | |
| | ARQSDDTYYYWGYFDYWGQGTMVT | | GQGTKLEIKRTVAAPSVFIFPPSDEQ | |
| | VSSASTKGPSVFPLAPSSKSTSGG | | LKSGTASVVCLLNNFYPREAKVQWKV | |
| | TAALGCLVKDYFPEPVTVSWNSGA | | DNALQSGNSQESVTEQDSKDSTYS | |
| | LTSGVHTFPAVLQSSGLYSLSSVV | | LSSTLTLSKADYEKHKVYACEVTHQG | |
| | TVPSSSLGTQTYICNVNHKPSNTK | | LSSPVTKSFNRGEC | |
| | VDKKVEPKSCDKTHTCPPCPAPEA | | | |
| | AGGPSVFLFPPKPKDTLMISKTPE | | | |
| | VTCVVVDVSHEDPEVKENWYVDGV | | | |
| | EVHNAKTKPREEDYNSTYRVVSVL | | | |
| | TVLHQDWLNGKEYKCKVSNKALPA | | | |
| | PIEKTISKAKGQPREPQVYTLPPS | | | |
| | REEMTKNQVSLTCLVKGFYPSDTA | | | |
| | VEWESNGQPENNYKTTPPVLDSDG | | | |
| | SFFLYSKLTVDKSRWQQGNVFSCS | | | |
| | VMHEALHNHYTQKSLSLSPGK | | | |

The binding kinetics of the recombinant anti-CD98 chimeric antibodies for purified recombinant CD98 protein (extracellular domain, ECD; SEQ ID NO: 126 and 127)), as described in Example 3, were determined by surface plasmon resonance-based measurements, results are shown in Table 24.

TABLE 24
Biacore Kinetics of Anti-CD98 Humanized Antibodies
Binding to Human and Cynomolgus Monkey CD98

| | Kinetics on Biacore | | | | | |
|---|---|---|---|---|---|---|
| | huCD98 ECD | | | cyCD98 ECD | | |
| Humanized | kₐ | k_{d} | K_{D} | kₐ | k_{d} | K_{D} |
| Clone | (M⁻¹s⁻¹) | (s⁻¹) | (M) | (M⁻¹s⁻¹) | (s⁻¹) | (M) |
| huAb101 | 2.9E+05 | 1.8E−03 | 6.1E−09 | 3.0E+04 | 6.7E−04 | 2.3E−08 |
| huAb102 | 3.3E+05 | 1.8E−03 | 5.5E−09 | 3.2E+04 | 6.8E−04 | 2.1E−08 |
| haAb103 | 1.8E+05 | 5.9E−04 | 3.4E−09 | 3.1E+04 | 4.4E−04 | 1.4E−08 |
| huAb104 | 4.5E+05 | 9.1E−04 | 2.0E−09 | 3.4E+04 | 4.8E−04 | 1.4E−08 |
| huAb105 | 1.4E+05 | 2.4E−05 | 1.7E−10 | 3.8E+04 | 5.3E−05 | 1.4E−09 |
| huAb106 | 1.4E+05 | 2.2E−05 | 1.6E−10 | 4.3E+04 | 9.5E−05 | 2.2E−09 |
| huAb107 | 1.1E+05 | 3.8E−05 | 3.3E−10 | 3.3E+04 | 8.7E−05 | 2.7E−09 |
| huAb108 | 7.5E+04 | 4.1E−05 | 5.5E−00 | 2.9E+04 | 1.4E−04 | 4.6E−09 |
| huAb109 | 1.6E+05 | 1.7E−04 | 1.1E−09 | 2.6E+04 | 3.1E−04 | 1.2E−08 |
| huAb110 | 1.3E+05 | 3.0E−04 | 2.3E−09 | 2.3E+04 | 5.1E−04 | 2.3E−08 |

hu = human;
cyno = cynomolgus monkey;
ECD = extracellular domain;
E + Y = ×10^{Y};
E − Y = ×10^{-Y}

### Example 15. Some Framework Re-Engineered Anti-CD98 mAbs Have Improved Conjugation Properties with Bcl-xL Inhibitor

These re-engineered humanized anti-CD98 mAbs were tested for conjugation with Bcl-xL inhibitor payload CZ and TX according to Method E, as set forth in Example 5 (Table 25 and 26), huAb108 and huAb110 behave the best for conjugation with CZ and TX payloads, in terms of conjugation efficiency as reflected by DAR (drugs/antibody ratio), estimated recovery based on concentration, and low level of aggregation as measured by size exclusion chromatography. Procedures for DAR and percent aggregate determination are described above in Example 5.

TABLE 25
Synthon CZ Conjugation of Re-engineered
Humanized Anti-CD98 mAbs

| Engineered | DAR | % | Estimated |
|---|---|---|---|
| Humanized | by | Aggregates | Recovery |
| clone | MS | by SEC | % |
| huAb101 | 4.5 | 1.8 | 71 |
| huAb102 | 4.5 | 3.9 | 73 |
| huAb103 | 4.4 | 15.9 | 79 |
| huAb104 | 3.6 | 17.6 | 97 |
| huAb105 | 1.2 | 1.4 | 30 |
| huAb106 | 3.1 | 3.3 | 48 |
| huAb107 | 2.2 | 1.9 | 39 |
| huAb108 | 3.6 | 3.9 | 94 |
| huAb109 | 2.1 | 2.5 | 48 |
| huAb110 | 3.6 | 4.6 | 88 |

TABLE 26
Synthon TX Conjugation of Re-engineered
Humanized Anti-CD98 mAbs

| Engineered | DAR | % | Estimated | |
|---|---|---|---|---|
| Humanized | by | Aggregates | Recovery | |
| clone | MS | by SEC | % | Observed Issues |
| huAb101 | 3.5 | 0.3 | 46 | Low recovery |
| huAb102 | 3.5 | 0.5 | 43 | Low recovery |
| huAb103 | 3.8 | 0.9 | 54 | Low recovery |
| huAb104 | 3.5 | 1.1 | 57 | Low recovery |
| huAb105 | 1.7 | 0.6 | 18 | Inefficient |
| | | | | conjugation; |
| | | | | very low recovery |
| huAb106 | 3.1 | 1.4 | 51 | Low recovery |
| huAb107 | 3.1 | 1.5 | 51 | Low recovery |
| huAb108 | 3.0 | 1.3 | 81 | |
| huAb109 | 2.9 | 1.0 | 45 | Low recovery |
| huAb110 | 3.2 | 1.4 | 84 | |

Note that the VH region of huAb106, huAb108 and huAb110 both contain an Asparagine (N) at the position of residue 74 (Table 19) that results in additional N-glycosylation of these two mAbs. This Asparagine (N) in the VH of huAb106, huAb108 and huAb110 was mutated to Threonine (T), resulting in mAbs huAbv106v1, huAb108v1 and huAb110v1, respectively (Table 22), huAb108v1 and huAb110 v1 are no longer optimal for conjugation with the Bcl-xL inhibitor synthons CZ and TX according to Method E, as described in Example 5.

### Example 16. In vitro Potency of Bcl-xL Inhibitor ADCs Derived from Selected Re-Engineered Anti-CD98 mAbs

huAb102, huAb104, huAb108, huAb110 anti-CD98 mAbs were selected to be conjugated with several Bcl-xL Inhibitor synthons according to Method G, as described in Example 5. The activities of these ADCs were tested in growth inhibition assays in the Molt-4 human acute lymphoblastic leukemia cell line. Briefly, 5000 Molt-4 cells per well in 96-well plates were treated with ADCs in serial dilution for 72 hours. The number of viable cells was determined by the ATPlite 1step reagent (PerkinElmer 6016739) as instructed by the manufacturer. Data was analyzed using Graphpad Prism software and IC₅₀ values were reported as the concentration of ADC to achieve 50% inhibition of cell proliferation (Table 27).

TABLE 27
In vitro Potency of Bcl-xL Inhibitor ADCs Derived from
Re-engineered Humanized Anti-CD98 mAbs

| | | | | | % Cell | |
|---|---|---|---|---|---|---|
| | Synthetic | DAR by | % agg by | Molt4 | Viability at | |
| ADC | Method | MS | SEC | IC₅₀ (nM) | 50 nM | |
| CC98 (CL-huAb102)-CZ | G | 3.3 | 5.2 | 0.015 | 1.0 | |
| CD98 (CL-huAb104)-CZ | G | 3.3 | 12.7 | 0.023 | 0.9 | |
| CD98 (CL-huAb108)-CZ | G | 2.9 | 4.8 | 0.068 | 1.1 | |
| CD98 (CL-huAb110)-CZ | G | 4.1 | 4.9 | 0.064 | 0.8 | |
| MSL109-CZ | G | 3.2 | 0.5 | >50 | 90.1 | |
| CD98 (CL-huAb102)-TX | G | 2.0 | 1.0 | 0.05 | 2.8 | |
| CD98 (CL-huAb104)-TX | G | 2.6 | 0.7 | 0.06 | 4.3 | |
| CD98 (CL-huAb108)-TX | G | 2.9 | 2.3 | 0.15 | 3.0 | |
| CD98 (CL-huAb110)-TX | G | 2.0 | 2.4 | 0.14 | 2.5 | |
| MSL109-TX | G | 2.7 | 0 | >50 | 91.1 | |
| CD98 (CL-huAb102)-TV | G | 3.9 | 1.7 | 0.02 | 1.5 | |
| CD98 (CL-huAb104)-TV | G | 4.1 | 2.6 | 0.03 | 1.4 | |
| CD98 (CL-huAb108)-TV | G | 3.3 | 1.6 | 0.08 | 1.1 | |
| CD98 (CL-huAb110)-TV | G | 3.0 | 2 | 0.09 | 1.0 | |
| MSL109-TV | G | 3.6 | 0 | >50 | 91.4 | |
| CD98 (CL-huAb102)-YY | G | 3.4 | 6.4 | 0.05 | 3.1 | |
| CD98 (CL-huAb104)-YY | G | 2.1 | 12.6 | 0.03 | 2.7 | |
| CD98 (CL-huAb108)-YY | G | 1.8 | 15.5 | 0.14 | 2.9 | |
| CD98 (CL-huAb110)-YY | G | 1.9 | 15.4 | 0.13 | 1.9 | |
| MSL109-YY | G | 2.9 | 0 | >50 | 92.1 | |
| CD98 (CL-huAb102)-AAA | G | 1.4 | 7.4 | 0.05 | 2.4 | |
| CD98 (CL-huAb104)-AAA | G | 1.9 | 11.7 | 0.04 | 2.2 | |
| CD98 (CL-huAb108)-AAA | G | 1.3 | 17.9 | 0.18 | 2.9 | |
| CD98 (CL-huAb110)-AAA | G | 1.0 | 15.2 | 0.16 | 1.9 | |
| MSL109-AAA | G | 1.9 | 13.7 | >50 | 96.5 | |
| CD98 (CL-huAb102)-AAD | G | 3.0 | 1.3 | 0.024 | 2.2 | |
| CD98 (CL-huAb104)-AAD | G | 3.0 | 2.3 | 0.028 | 2.5 | |
| CD98 (CL-huAb108)-AAD | G | 2.6 | 3.3 | 0.091 | 1.8 | |
| CD98 (CL-huAb110)-AAD | G | 3.2 | 2.9 | 0.074 | 1.5 | |
| MSL109-AAD | G | 3.0 | 0.4 | >50 | 97.4 | |
| CD98 (CL-huAb102)-LB | A | 2 | 4.5 | 0.053 | 14.8 | |
| CD98 (CL-huAb104)-LB | A | 2.2 | 13.6 | 0.062 | 3.5 | |
| CD98 (CL-huAb110)-LB | A | 2.1 | 18 | 0.208 | 1.9 | |
| MSL109-LB | A | 1.8 | 0 | 6.146 | 2.2 | |
| CD98 (CL-huAb102)-WD | E | 1.4 | 0 | 0.109 | 14.3 | |
| CD98 (CL-huAb104)-WD | E | 2 | 0 | 0.067 | 16.4 | |
| CD98 (CL-huAb110)-WD | E | 1.8 | 4.8 | 0.226 | 10.7 | |
| MSL109-WD | E | 2.9 | 0 | 19.1 | 6.9 | |
| CD98 (CL-huAb102)-ZT | G | 1.5 | 7.8 | 0.040 | 3.4 | |
| CD98 (CL-huAb104)-ZT | G | 1.7 | 12.1 | 0.042 | 3.3 | |
| CD98 (CL-huAb108)-ZT | G | 1.8 | 13.5 | 0.144 | 4.5 | |
| CD98 (CL-huAb110)-ZT | G | 0.6 | 13 | 0.156 | 3.5 | |
| MSL109-ZT | G | 2.3 | 7.5 | >50 | 96.3 | |
| CD98 (CL-huAb102)-ZZ | G | 0.8 | 7.8 | 0.072 | 6.3 | |
| CD98 (CL-huAb104)-ZZ | G | 1.1 | 10.4 | 0.061 | 6.5 | |
| CD98 (CL-huAb108-ZZ | G | 0.5 | 19.4 | 0.199 | 5.8 | |
| CD98 (CL-huAb110)-ZZ | G | 1.0 | 15 | 0.252 | 4.3 | |
| MSL09-ZZ | G | 1.4 | 15 | >50 | 99.7 | |
| CD98 (CL-huAb102)-XW | G | 2.8 | 2.4 | 0.074 | 32.0 | |
| CD98 (CL-huAb104)-XW | G | 3.1 | 3.1 | 0.102 | 31.1 | |
| CD98 (CL-huAb108)-XW | G | 3.5 | 6.8 | 0.281 | 20.9 | |
| CD98 (CL-huAb110)-XW | G | 3.2 | 7 | 0.308 | 19.6 | |
| MSL109-XW | G | 3.3 | 3.7 | >50 | 97.3 | |
| CD98 (CL-huAb102)-SE | A | 2.2 | 0 | 0.107 | 19.7 | |
| CD98 (CL-huAb104)-SE | A | 2.4 | 0 | 0.149 | 17.1 | |
| CD98 (CL-huAb110)-SE | A | 1.9 | 3.5 | 0.502 | 5.7 | |
| MSL109-SE | A | 3.6 | 33.4 | 23.69 | 10.6 | |
| CD98 (CL-huAb102)-SR | A | 2.1 | 12.2 | 0.030 | 12.7 | |
| CD98 (CL-huAb104)-SR | A | 2.2 | 31.4 | 0.045 | 2.7 | |
| CD98 (CL-huAb110)-SR | A | .0.7 | 17.1 | 0.332 | 11.0 | |
| MSL109-SR | A | 1.8 | 2.3 | 44.300 | 44.2 | |
| CD98 (CL-huAb102)-YG | E | 1.1 | 0 | 1.210 | 31.9 | |
| CD98 (CL-huAb104)-YG | E | 1.3 | 0 | 2.667 | 25.3 | |
| CD98 (CL-huAb110)-YG | E | 2.8 | 2.4 | 0.493 | 10.0 | |
| MSL109-YG | E | 3.1 | 13.2 | 22.43 | 14.6 | |
| | General | DAR | Dose | Regimen/ | | TGIₘₐₓ |
| Drug | Method | by MS | (mg/kg/day) | Route | N | (%) |
| [symbol] | [symbol] | [symbol] | [symbol] | [symbol] | [symbol] | [symbol] |

| | | | | | % Cell |
|---|---|---|---|---|---|
| | Synthetic | DAR by | % agg by | Molt4 | Viability at |
| ADC | Method | MS | SEC | IC₅₀ (nM) | 50 nM |
| CD98 (CL-huAb102)-KZ | A | 2.8 | 0.7 | 0.062 | 20.7 |
| CD98 (CL-huAb104)-KZ | A | 2.6 | 6.3 | 0.089 | 21.2 |
| CD98 (CL-huAb102)-KZ | A | 2.2 | 4.4 | 0.350 | 16.2 |
| MSL109-KZ | A | 2.5 | 18 | >50 | 91.9 |

MSL109 is a humanized IgG1 antibody that binds to cytomegalovirus (CMV) glycoprotein H. It is used as a negative control mAb. [symbol]
indicates data missing or illegible when filed

### Example 17. In Vivo Potency of Bcl-xL Inhibitor ADCs Derived from Selected Re-Engineered Anti-CD98 mAbs

The in vivo anti-tumor efficacy of selected humanized anti-CD98 mAb conjugates were tested in NCI—H146 (human small cell lung cancer) xenograft model, as described in Example 6. Tumor growth inhibition was reported as TGIₘₐₓ in Table 28.

TABLE 28
Inhibition of NCI-H146 Xenograft Tumor Growth after
Treatment with a Single Dose of CD98-targeting Bcl-xLi ADC

| | | | | | | |
|---|---|---|---|---|---|---|
| Ab095 | | | 10 | QDx1/IP | 8 | 0 |
| CD98 (CL-huAb102)-CZ | A | 2.9 | 10 | QDx1/IP | 8 | 93 |
| CD98 (CL-huAb102)-TX | E | 1.8 | 10 | QDx1/IP | 8 | 92 |
| CD98 (CL-huAb102)-XW | E | 2.6 | 10 | QDx1/IP | 8 | 55 |
| CD98 (CL-huAb102)-AAA | E | 1.8 | 10 | QDx1/IP | 8 | 69 |
| CD98 (CL-huAb104)-CZ | A | 2.6 | 10 | QDx1/IP | 8 | 90 |
| CD98 (CL-huAb104)-AAA | E | 2.2 | 10 | QDx1/IP | 8 | 61 |
| CD98 (CL-huAb108)-CZ | A | 3.2 | 10 | QDx1/IP | 8 | 93 |
| CD98 (CL-huAb108)-AAA | E | 3.0 | 10 | QDx1/IP | 8 | 61 |
| CD98 (CL-huAb110)-CZ | A | 2.9 | 10 | QDx1/IP | 8 | 92 |

### Example 18. In Vivo Potency of Bcl-xL Inhibitor ADCs Derived from Selected Re-Engineered Anti-CD98 mAbs

The in vivo efficacy of anti-CD98 huAb108 conjugated to synthon TX, prepared according to General Method E with a DAR 2.3, was determined in the xenografted human lung carcinoma models A549 and NCI—H460. The cell lines A549 and NCI—H460 were obtained from the American Type Culture Collection (ATCC, Manassas, Va.). A549 cell line was further passaged in mice as flank xenograft to improve xenograft tumor growth, resulting in the A549-FP3 line. Cells were cultured as monolayers in RPMI-1640 culture media (Invitrogen, Carlsbad, Calif.) supplemented with 10% Fetal Bovine Serum (FBS, Hyclone, Logan, Utah). To generate xenografts, 5×10⁶ (A549 and NCI—H460) viable cells were inoculated subcutaneously into the right flank of immune deficient female SCID-bg mice (Charles River Laboratories, Wilmington, Mass.). The injection volume was 0.2 ml and composed of 1:1 S-MEM:Matrigel Matrigel (BD, Franklin Lakes, N.J.). Tumors were size matched at approximately 223 mm³. Antibodies, conjugates, and docetaxel were formulated in 0.9% sodium chloride for injection. Injection volume did not exceed 200 μl. Therapy began within 24 hours after size matching of the tumors. Mice weighed approximately 21 g at the onset of therapy. Tumor volume was estimated two to three times weekly. Measurements of the length (L) and width (W) of the tumor were taken via electronic caliper and the volume was calculated according to the following equation: V=L×W²/2. Mice were euthanized when tumor volume reached 3,000 mm³ or skin ulcerations occurred. Eight mice were housed per cage. Food and water were available ad libitum. Mice were acclimated to the animal facilities for a period of at least one week prior to commencement of experiments. Animals were tested in the light phase of a 12-hour light: 12-hour dark schedule (lights on at 06:00 hours). Anti-CD98 conjugates (10 mg/kg) were administered as a single dose (QDx1) intraperitoneally. Docetaxel (7.5 mg/kg) was administered as a single dose (QDx1) intravenously. A human IgG control antibody (Ab095) was used as a negative control agent.

To refer to efficacy of therapeutic agents, parameters of amplitude (TGIₘₐₓ), durability (TGD) are used. The efficacy of inhibition of A549 and NCI—H460 xenograft growth with CD98-targeted ADCs is illustrated by Table 29 and 30. In the tables, to refer to efficacy, parameters of amplitude (TGIₘₐₓ) and durability (TGD) of therapeutic response are used. TGIₘₐₓ is the maximum tumor growth inhibition during the experiment. Tumor growth inhibition is calculated by 100*(1−T_{c}/Cᵥ) where T, and Cᵥ are the mean tumor volumes of the treated and control groups, respectively. TGD or tumor growth delay is the extended time of a treated tumor needed to reach a volume of 1 cm³ relative to the control group. TGD is calculated by 100*(Tₜ/Cᵣ−1) where Tₜ and Cₜ are the median time periods to reach 1 cm³ of the treated and control groups, respectively.

TABLE 29
Inhibition of A549 FP3 Xenograft Tumor Growth by a CD98-targeting
Bcl-xLi ADC With or Without the Combination of Docetaxel

| | Dose | | | TGIₘₐₓ | TGD |
|---|---|---|---|---|---|
| Drug | (mg/kg/day) | Regimen | N | (%) | (%) |
| Ab095 | 8 | QDx1/IP | 8 | 0 | 0 |
| Docetaxel | 7.5 | QDx1/IV | 8 | 62 | 80 |
| CD98 (CL- | 10 | QDx1/IP | 8 | 57 | 60 |
| huAb108)-TX | | | | | |
| Docetaxel + CD98 | 7.5 + 10 | QDx1/IV + | 8 | 87 | 127 |
| (CL-huAb108)-TX | | QDx1/IP | | | |

TABLE 30
Inhibition of NCI-H460 Xenograft Tumor Growth by a CD98-targeting
Bcl-xLi ADC With or Without the Combination of Docetaxel

| | Dose | | | TGIₘₐₓ | TGD |
|---|---|---|---|---|---|
| Drug | (mg/kg/day) | Regimen | N | (%) | (%) |
| Ab095 | 8 | QDx1/IP | 8 | 0 | 0 |
| Docetaxel | 7.5 | QDx1/IV | 8 | 35 | 30 |
| CD98 (CL- | 10 | QDx1/IP | 8 | 35 | 22 |
| huAb108)-TX | | | | | |
| Docetaxel + CD98 | 7.5 + 10 | QDx1/IV + | 8 | 66 | 61 |
| (CL-huAb108)-TX | | QDx1/IP | | | |

SEQUENCE SUMMARY

| SEQ ID | |
|---|---|
| NO: | Description |
| 1 | chAb1 VH amino acid sequence |
| 2 | chAb1, chAb4 VH CDR1 amino acid sequence |
| 3 | chAb1 VH CDR2 amino acid sequence |
| 4 | chAb1, chAb2, chAb4 VH CDR3 amino acid sequence |
| 5 | chAb1 VL amino acid sequence |
| 6 | chAb1 VL CDR1 amino acid sequence |
| 7 | chAb1, chAb2, chAb3, chAb4, chAb5, chAb6, chAb7, chAb9, chAb13, huAb3, huAb3v1, |
| | huAb3v2, huAb101, huAb102, huAb103, huAb104 VL CDR2 amino acid sequence |
| 8 | chAb1, chAb4 VL CDR3 amino acid sequence |
| 9 | chAb2 VH amino acid sequence |
| 10 | chAb2 VH CDR1 amino acid sequence |
| 11 | chAb2, chAb3, chAb5, huAb3 VH CDR2 amino acid sequence |
| 12 | chAb2 VL amino acid sequence |
| 13 | chAb2, chAb3, chAb4, chAb5, huAb3, huAb3v1, huAb3v2, huAb101, huAb102, huAb103, |
| | huAb104 VL CDR1 amino acid sequence |
| 14 | chAb2 VL CDR3 amino acid sequence |
| 15 | cbAb3 VH amino acid sequence |
| 16 | chAb3, huAb3, huAb3v1, huAb3v2, huAb101, huAb102, huAb103, huAb104 VH CDR1 |
| | amino acid sequence |
| 17 | chAb3, huAb3, huAb3v1, chAb3v2, huAb101, huAb102, huAb103, huAb104 VH CDR3 |
| | amino acid sequence |
| 18 | chAb3 VL amino acid sequence |
| 19 | chAb3, huAb3, huAb3v1, chAb3v2, huAb102, huAb102, huAb103, buAb104 VL CDR3 |
| | amino acid sequence |
| 20 | chAb4 VH amino acid sequence |
| 21 | chAb4 VH CDR2 amino acid sequence |
| 22 | chAb4 VL amino acid sequence |
| 23 | chAb5 VH amino acid sequence |
| 24 | chAb5 VH CDR1 amino acid sequence |
| 25 | chAb5 VH CDR3 amino acid sequence |
| 26 | chAb5 VL amino acid sequence |
| 27 | cbAb5 VL CDR3 amino acid sequence |
| 28 | cbAb6 VH amino acid sequence |
| 29 | chAb6, cbAb7 VH CDR1 amino acid sequence |
| 30 | chAb6, chAb9 VH CDR2 amino acid sequence |
| 31 | chAb6 VH CDR3 amino acid sequence |
| 32 | chAb6 VL amino acid sequence |
| 33 | chAb6, chAb7 VL CDR1 amino acid sequence |
| 34 | chAb6, chAb7 VL CDR3 amino acid sequence |
| 35 | chAb7 VH amino acid sequence |
| 36 | chAb7 VH CDR2 amino acid sequence |
| 37 | chAb7, chAb9 VH CDR3 amino acid sequence |
| 38 | chAb7 VL amino acid sequence |
| 39 | chAb8 VH amino acid sequence |
| 40 | chAb8, chAb10, chAb11, chAb12 VH CDR1 amino acid sequence |
| 41 | chAb8, chAb12 VH CDR2 amino acid sequence |
| 42 | chAb8, chAb11 VH CDR3 amino acid sequence |
| 43 | chAb8 VL amino acid sequence |
| 44 | chAb8, chAb10, chAb12 VL CDR1 amino acid sequence |
| 45 | chAb8, chAb10, chAb11, chAb12, chAb15, huAb15, huAb15v1, huAb15v2, hAb15v3, |
| | hAb15v4, hAb15v5, huAb15v6, buAb15v7, huAb105, huAb106, huAb107, huAb108, |
| | huAb109, huAb110, huAb106v1, huAb108v1, huAb110v1 VL CDR2 amino acid sequence |
| 46 | chAb8, chAb10, chAb11, chAb12 VL CDR3 amino acid sequence |
| 47 | chAb9 VH amino acid sequence |
| 48 | chAb9 VH CDR1 amino acid sequence |
| 49 | chAb9 VL amino acid sequence |
| 50 | chAb9 VL CDR1 ammo acid sequence |
| 51 | chAb9 VL CDR3 amino acid sequence |
| 52 | chAb10 VH amino acid sequence |
| 53 | chAb10 VH CDR2 amino acid sequence |
| 54 | chAb10 VH CDR3 amino acid sequence |
| 55 | chAb10 VL amino acid sequence |
| 56 | chAb11 VH amino acid sequence |
| 57 | chAb11 VH CDR2 amino acid sequence |
| 58 | chAb11 VL amino acid sequence |
| 59 | chAb11 VL CDR1 amino acid sequence |
| 60 | chAb12 VH amino acid sequence |
| 61 | chAb12 VH CDR3 amino acid sequence |
| 62 | chAb12 VL amino acid sequence |
| 63 | chAb13 VH amino acid sequence |
| 64 | chAb13 VH CDR1 amino acid sequence |
| 65 | chAb13 VH CDR2 amino acid sequence |
| 66 | chAb13 VH CDR3 amino acid sequence |
| 67 | chAb13 VL amino acid sequence |
| 68 | chAb13 VL CDR1 amino acid sequence |
| 69 | chAb13 VL CDR3 amino acid sequence |
| 70 | chAb14 VH amino acid sequence |
| 71 | chAb14 VH CDR1 amino acid sequence |
| 72 | chAb14 VH CDR2 amino acid sequence |
| 73 | chAb14 VH CDR3 amino acid sequence |
| 74 | chAb14 VL amino acid sequence |
| 75 | chAb14 VL CDR1 amino acid sequence |
| 76 | chAb14 VL CDR2 amino acid sequence |
| 77 | chAb14 VL CDR3 amino acid sequence |
| 78 | chAb15 VH amino acid sequence |
| 79 | chAb15, huAb15, huAb15v1, huAb15v2, huAb15v3, huAb15v4, huAb15v5, huAb15v6, |
| | huAb15v7, huAb105, huAb106, huAb107, huAb108, huAb109, huAb110, huAb106v1, |
| | huAb108v1, huAb110v1 VH CDR1 amino acid sentience |
| 80 | chAb15, huAb15 VH CDR2 amino acid sequence |
| 81 | chAb15, huAb15 VH CDR3 amino acid sequence |
| 82 | chAb15 VL amino acid sequence |
| 83 | chAb15, huAb15, huAb15v1, huAb15v2, huAb15v3, huAb15v4, huAb15v5, huAb15v6, |
| | huAb15v7, huAb105, huAb106, huAb107, huAb108, buAb109, huAb110, huAb106v1, |
| | huAb108v1, huAb110v1 VL CDR1 amino acid sequence |
| 84 | chAb15, huAb15 VL CDR3 amino acid sequence |
| 85 | huAb3 VH amino acid sequence; bCL-Ab3VH.1a amino acid sequence |
| 86 | huAb3v1 VH amino acid sequence |
| 87 | huAb3v1, huAb101, huAb102 VH CDR2 amino acid sequence |
| 88 | huAb3, huAb3v1, huAb3v2 VL amino acid sequence |
| 89 | huAb3v2 VH amino acid sequence |
| 90 | huAb3v2, huAb103, huAb104 VH CDR2 amino acid sequence |
| 91 | huAb15v1 VH amino acid sequence |
| 92 | huAb15v1, huAb15v2, huAb15v3, huAb105, huAb106, huAb107, huAb108, huAb106v1, |
| | huAb108v1 VH CDR2 amino acid sequence |
| 93 | huAb15v1, huAb105, huAb106, huAb106v1 VH CDR3 amino acid sequence |
| 94 | huAb15v1, buAb15v2, huAbv4 VL amino acid sequence |
| 95 | huAb15v1, huAb15v2, huAb15v4, huAb105, huAb106, huAb107, huAb108, huAb106v1, |
| | huAb108v1 VL CDR3 amino acid sequence |
| 96 | huAb15v2, hAb15v3 VH amino acid sequence |
| 97 | huAb15v2, huAb15v3, huAb15v4, huAb15v5, huAb15v6, huAb15v7, huAb107, huAb108, |
| | huAb109, huAb110, huAb108v1, huAb110v1 VH CDR3 amino acid sequence |
| 98 | huAb15v3, hAb15v7 VL amino acid sequence |
| 99 | huAb15v4, huAb15v5 VH amino acid sequence |
| 100 | huAb15v4, huAb15v5VH CDR2 amino acid sequence |
| 101 | huAb15v5, huAb15v6 VL amino acid sequence |
| 102 | huAb15v5, huAb15v6, huAb109, huAb110, huAb110v1 VL CDR3 amino acid sequence |
| 103 | huAb15v6, huAb15v7 VH amino acid sequence |
| 104 | huAb15v6, huAB15v7, huAb109, huAb110, huAb110v1 VH CDR2 amino acid sequence |
| 105 | huAb15v3, huAb15v7 VL CDR3 amino acid sequence |
| 106 | huAb101 VH amino acid sequence |
| 107 | huAb101, huAb102, huAb103, huAb104 VL amino acid sequence |
| 108 | huAb102 VH amino acid sequence |
| 109 | huAb103 VH amino acid sequence |
| 110 | huAb104 VH amino acid sequence |
| 111 | huAb105 VH amino acid sequence |
| 112 | huAb105, huAb106, huAb107, huAb108, huAb106v1, huAb108v1 VL amino acid |
| 113 | huAb106 VH amino acid sequence |
| 114 | huAb107 VH amino acid sequence |
| 115 | huAb108 VH amino acid sequence |
| 116 | huAb109 VH amino acid sequence |
| 117 | huAb109 VL, huAb110, huAb110v1 VL amino acid sequence |
| 118 | huAb110 VH amino acid sequence |
| 119 | huAb106v1 VH amino acid sequence |
| 120 | huAb108v1 VH amino acid sequence |
| 121 | huAb110v1 VH amino acid sequence |
| 122 | huAb15 VH amino acid sequence; hCL-Ab15VH.1a amino acid sequence |
| 123 | huAb15 VL amino acid sequence; hCL-Ab15VL.1a amino acid sequence |
| 124 | Amino acid sequence of CD98 |
| 125 | Amino acid sequence of the extracellular domain of CD98 (amino acids |
| | 206-630 of SEQ ID NO: 124) |
| 126 | Human CD98 ECD with N-terminal His-tag |
| 127 | Cynomolgus monkey CD98 ECD with C-terminal His-tag |
| 128 | Human CD98 ECD with C-terminal His-tag |
| 129 | Cynomolgus monkey CD98 ECD with C-terminal His-tag |
| 130 | hCL-Ab3VH.1 amino acid sequence |
| 131 | hCL-Ab3VH.1b amino acid sequence |
| 132 | hCL-Ab3VH.1c amino acid sequence |
| 133 | hCL-Ab3VH.2 ammo acid sequence |
| 134 | hCL-Ab3VH.2a amino acid sequence |
| 135 | hCL-Ab3VH.2b amino acid sequence |
| 136 | hCL-Ab3VH.3 amino acid sequence |
| 137 | hCL-Ab3VH.3a amino acid sequence |
| 138 | hCL-Ab3VH.3b amino acid sequence |
| 139 | hCL-Ab3VH.3c amino acid sequence |
| 140 | hCL-Ab3VL.1 amino acid sequence |
| 141 | hCL-Ab3VL.1a amino acid sequence |
| 142 | hCL-Ab3VL.2 amino acid sequence |
| 143 | hCL-Ab15VH.1z amino acid sequence |
| 144 | hCL-Ab15VH.1 amino acid sequence |
| 145 | hCL-Ab15VH.2 amino acid sequence |
| 146 | hCL-Ab15VH.2a amino acid sequence |
| 147 | hCL-Ab15VH.3z amino acid sequence |
| 148 | hCL-Ab15VH.3 amino acid sequence |
| 149 | hCL-Ab15VH.3a amino acid sequence |
| 150 | hCL-Ab15VH.3b amino acid sequence |
| 151 | hCL-Ab15VL.1 amino acid sequence |
| 152 | hCL-Ab15VL.2 amino acid sequence |
| 153 | hCL-Ab15VL.2a amino acid sequence |
| 154 | Ig gamma-1 constant region |
| 155 | Ig gamma-1 constant region mutant |
| 156 | Ig Kappa constant region |
| 157 | Ig Lambda constant region |
| 158 | huAb102 Heavy Chain amino acid sequence |
| 159 | huAb102 Light Chain amino acid sequence |
| 160 | huAb104 Heavy Chain amino acid sequence |
| 161 | huAb104 Light Chain amino acid sequence |
| 162 | huAb108 Heavy Chain amino acid sequence |
| 163 | huAb108 Light Chain amino acid sequence |
| 164 | huAb110 Heavy Chain amino acid sequence |
| 165 | huAb110 Heavy Chain amino acid sequence |
| 166 | Cleavable peptide Gly-Phe-Leu-Gly |
| 167 | Cleavable peptide Ala-Leu-Ala-Leu |

### INCORPORATION BY REFERENCE

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. An isolated anti-CD98 antibody, wherein the antibody comprises a heavy chain variable region comprising
a CDR1 having the amino acid sequence of SEQ ID NO: 16 or SEQ ID NO: 79,
a CDR2 having the amino acid sequence of SEQ ID NO: 87, SEQ ID NO: 90, SEQ ID NO: 92, or SEQ ID NO: 104;
a CDR3 having the amino acid sequence of SEQ ID NO: 17 or SEQ ID NO: 97; and a light chain variable region comprising
a CDR1 having the amino acid sequence of SEQ ID NO: 13 or SEQ ID NO: 83;
a CDR2 having the amino acid sequence of SEQ ID NO: 7 or SEQ ID NO: 45;
a CDR3 having the amino acid sequence of SEQ ID NO: 19, SEQ ID NO: 95, or SEQ ID NO: 102.

2. The anti-CD98 antibody according to claim 1, comprising a heavy chain variable domain comprising an amino acid sequence set forth in SEQ ID NO: 108, SEQ ID NO: 110, SEQ ID NO: 115, or SEQ ID NO: 118, and a light chain variable domain comprising an amino acid sequence set forth in SEQ ID NO: 107, SEQ ID NO: 112, or SEQ ID NO: 117.

3. The anti-CD98 antibody according to claim 1, wherein the antibody comprises a heavy chain immunoglobulin constant domain of a human IgG constant domain, wherein the human IgG1 constant domain comprises an amino acid sequence of SEQ ID NO:154 or SEQ ID NO:155.

4. The anti-CD98 antibody according to claim 1, wherein the antibody comprises a heavy chain comprising the amino acid sequence set forth in SEQ ID NO: 158, SEQ ID NO: 160, SEQ ID NO: 162, or SEQ ID NO: 164, and a light chain comprising the amino acid sequence set forth in SEQ ID NO: 159, SEQ ID NO: 161, SEQ ID NO: 163, or SEQ ID NO: 165.

5. A pharmaceutical composition comprising the anti-CD98 antibody of claim 1, and a pharmaceutically acceptable carrier.

6. An anti-CD98 Antibody Drug Conjugate (ADC) comprising an anti-CD98 antibody of claim 1 conjugated to a drug via a linker.

7. The ADC of claim 6, wherein the drug is an auristatin or a pyrrolobenzodiazepine (PBD).

8. The ADC of claim 6, wherein the drug is a Bcl-xL inhibitor.

9. An anti-human CD98 (hCD98) antibody drug conjugate (ADC) comprising a drug linked to an anti-human CD98 (hCD98) antibody via a linker, wherein the drug is a Bcl-xL inhibitor according to structural formula (IIa) or (IIb): 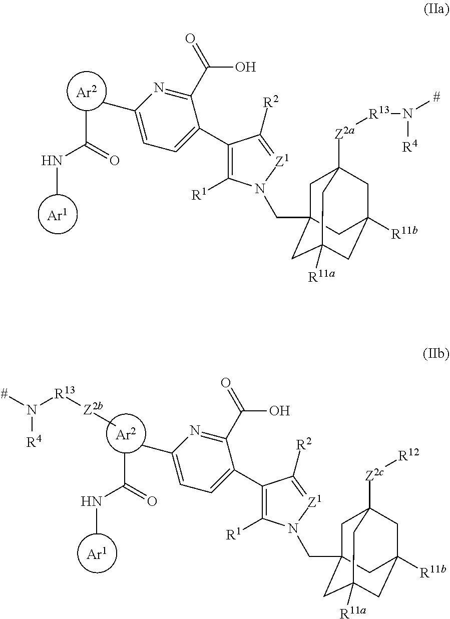
wherein:
Ar¹ is selected from 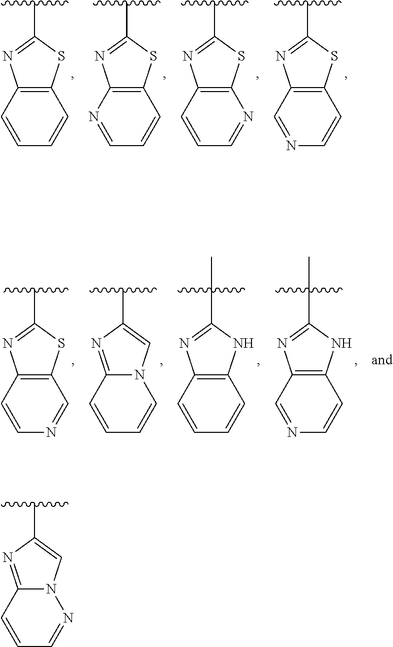
and is optionally substituted with one or more substituents independently selected from halo, hydroxy, nitro, lower alkyl, lower heteroalkyl, C₁₋₄alkoxy, amino, cyano and halomethyl;
Ar² is selected from 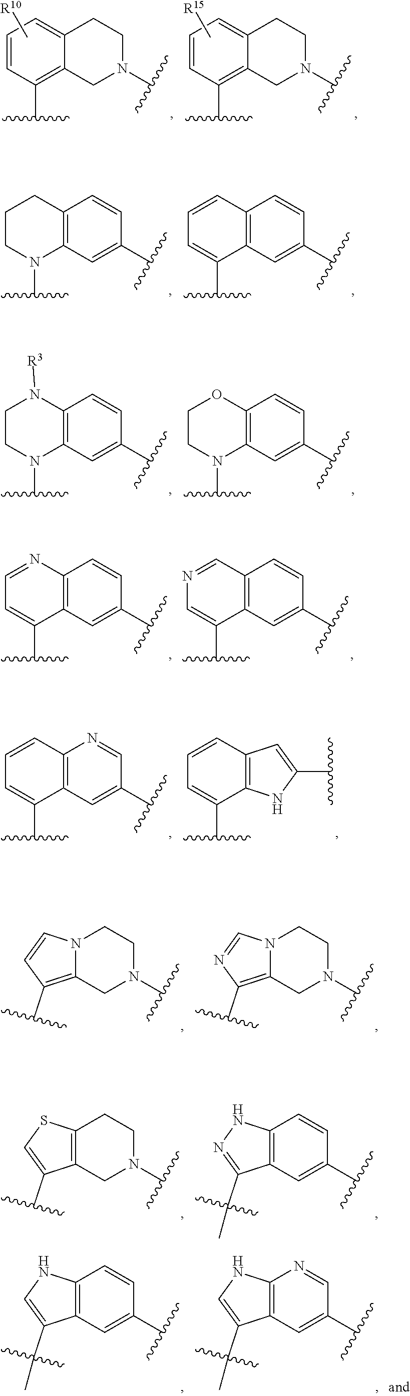 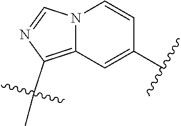 [SMILES: *c1ccn2cnc(*)c2c1]
and is optionally substituted with one or more substituents independently selected from halo, hydroxy, nitro, lower alkyl, lower heteroalkyl, C₁₋₄alkoxy, amino, cyano and halomethyl, wherein the #—N(R⁴)—R¹³—Z^{2b}— substituent of formula (IIb) is attached to Ar² at any Ar² atom capable of being substituted;
Z¹ is selected from N, CH, C-halo and C—CN;
Z^{2a}, Z^{2b}, and Z^{2c} are each, independent from one another, selected from a bond, NR⁶, CR^{6a}R^{6b}, O, S, S(O), SO₂, NR⁶C(O), NR^{6a}C(O)NR^{6b}, and NR⁶C(O)O;
R¹ is selected from hydrogen, methyl, halo, halomethyl, ethyl and cyano;
R² is selected from hydrogen, methyl, halo, halomethyl and cyano;
R³ is selected from hydrogen, lower alkyl and lower heteroalkyl;
R⁴ is selected from hydrogen, lower alkyl, monocyclic cycloalkyl, monocyclic heterocyclyl, and lower heteroalkyl or is taken together with an atom of R¹³ to form a cycloalkyl or heterocyclyl ring having between 3 and 7 ring atoms, wherein the lower alkyl, monocyclic cycloalkyl, monocyclic heterocyclyl, and lower heteroalkyl are optionally substituted with one or more halo, cyano, hydroxy, C₁₋₄alkoxy, monocyclic cycloalkyl, monocyclic heterocyclyl, C(O)NR^{6a}R^{6b}, S(O)₂NR^{6a}R^{6b}, NHC(O)CHR^{6a}R^{6b}, NHS(O)CHR^{6a}R^{b}, NHS(O)₂CHR^{6a}R^{6b}, S(O)₂CHR^{6a}R^{6b} or S(O)₂NH₂ groups;
R⁶, R^{6a} and R^{6b} are each, independent from one another, selected from hydrogen,
lower alkyl, lower heteroalkyl, optionally substituted monocyclic cycloalkyl and monocyclic heterocyclyl, or are taken together with an atom from R¹³ to form a cycloalkyl or heterocyclyl ring having between 3 and 7 ring atoms;
R¹⁰ is selected from cyano, OR¹⁴, SR¹⁴, SOR¹⁴, SO₂R¹⁴, SO₂NR^{14a}R^{14b}, NR^{14a}R^{14b}, NHC(O)R¹⁴ and NHSO₂R¹⁴;
R^{11a} and R^{11b} are each, independently of one another, selected from hydrogen, halo, methyl, ethyl, halomethyl, hydroxyl, methoxy, CN, and SCH₃;
R¹² is selected from hydrogen, halo, cyano, lower alkyl, lower heteroalkyl, cycloalkyl, and heterocyclyl, wherein the alkyl, heteroalkyl, cycloalkyl, and heterocyclyl are optionally substituted with one or more halo, cyano, C₁₋₄alkoxy, monocyclic cycloalkyl, monocyclic heterocyclyl, NHC(O)CHR^{6a}R^{6b}, NHS(O)CHR^{6a}R^{6b}, NHS(O)₂CHR^{6a}R^{6b} or S(O)₂CHR^{6a}R^{6b} groups;
R¹³ is selected from a bond, optionally substituted lower alkylene, optionally substituted lower heteroalkylene, optionally substituted cycloalkyl or optionally substituted heterocyclyl;
R¹⁴ is selected from hydrogen, optionally substituted lower alkyl and optionally substituted lower heteroalkyl;
R^{14a} and R^{14b} are each, independently of one another, selected from hydrogen, optionally substituted lower alkyl, and optionally substituted lower heteroalkyl, or are taken together with the nitrogen atom to which they are bonded to form an optionally substituted monocyclic cycloalkyl or monocyclic heterocyclyl ring;
R¹⁵ is selected from hydrogen, halo, C₁₋₆ alkanyl, C₂₋₄ alkenyl, C₂₋₄ alkynyl, and C₁₋₄ haloalkyl and C₁₋₄ hydroxyalkyl, with the proviso that when R¹⁵ is present, R⁴ is not C₁₋₄ alkyl, C₂₋₄ alkenyl, C₂₋₄ alkynyl, C₁₋₄ haloalkyl or C₁₋₄ hydroxyalkyl, wherein the R⁴ C₁₋₆ alkanyl, C₂₋₄ alkenyl, C₂₋₄ alkynyl, C₁₋₄ haloalkyl and C₁₋₄ hydroxyalkyl are optionally substituted with one or more substituents independently selected from OCH₃, OCH₂CH₂OCH₃, and OCH₂CH₂NHCH₃; and
# represents a point of attachment to a linker.

10. The ADC of claim 9, which is a compound according to structural formula (I): 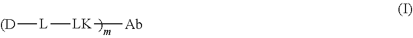
wherein:
D is the Bcl-xL inhibitor drug of formula (IIa) or (IIb);
L is the linker;
Ab is the anti-hCD98 antibody;
LK represents a covalent linkage linking the linker (L) to the anti-hCD98 antibody (Ab); and
m is an integer ranging from 1 to 20.

11. The ADC of claim 10, wherein the Bcl-xL inhibitor is selected from the group consisting of the following compounds modified in that the hydrogen corresponding to the # position of structural formula (IIa) or (IIb) is not present forming a monoradical:
6-[1-(1,3-benzothiazol-2-ylcarbamoyl)-1,2,3,4-tetrahydroquinolin-7-yl]-3-[1-({3,5-dimethyl-7-[2-(methylamino)ethoxy]tricyclo[3.3.1.13,7]dec-1-yl}methyl)-5-methyl-1H-pyrazol-4-yl]pyridine-2-carboxylic acid;
6-[4-(1,3-benzothiazol-2-ylcarbamoyl)-3,4-dihydro-2H-1,4-benzoxazin-6-yl]-3-[1-({3,5-dimethyl-7-[2-(methylamino)ethoxy]tricyclo[3.3.1.13,7]dec-1-yl}methyl)-5-methyl-1H-pyrazol-4-yl]pyridine-2-carboxylic acid;
6-[4-(1,3-benzothiazol-2-ylcarbamoyl)-1-methyl-1,2,3,4-tetrahydroquinoxalin-6-yl]-3-[1-(({3,5-dimethyl-7-[2-(methylamino)ethoxy]tricyclo[3.3.1.13,7]dec-1-yl}methyl)-5-methyl-1H-pyrazol-4-yl]pyridine-2-carboxylic acid;
3-(1-{[3-(2-aminoethoxy)-5,7-dimethyltricyclo[3.3.1.13,7]dec-1-yl]methyl}-5-methyl-1H-pyrazol-4-yl)-6-[-(1,3-benzothiazol-2-ylcarbamoyl)-5,6-dihydroimidazo[1,5-a]pyrazin-7(8H)-yl]pyridine-2-carboxylic acid;
3-(1-{[3-(2-aminoethoxy)-5,7-dimethyltricyclo[3.3.1.13,7]dec-1-yl]methyl}-5-methyl-1H-pyrazol-4-yl)-6-[8-(1,3-benzothiazol-2-ylcarbamoyl)-5-hydroxy-3,4-dihydroisoquinolin-2(1H)-yl]pyridine-2-carboxylic acid;
6-[8-(1,3-benzothiazol-2-ylcarbamoyl)naphthalen-2-yl]-3-[1-({3,5-dimethyl-7-[2-(methylamino)ethoxy]tricyclo[3.3.1.13,7]dec-1-yl}methyl)-5-methyl-1H-pyrazol-4-yl]pyridine-2-carboxylic acid;
3-[1-({3,5-dimethyl-7-[2-(methylamino)ethoxy]tricyclo[3.3.1.13,7]dec-1-yl}methyl)-5-methyl-1H-pyrazol-4-yl]-6-[8-([1,3]thiazolo[5,4-b]pyridin-2-ylcarbamoyl)naphthalen-2-yl]pyridine-2-carboxylic acid;
3-[1-({3,5-dimethyl-7-[2-(methylamino)ethoxy]tricyclo[3.3.1.13,7]dec-1-yl}methyl)-5-methyl-1H-pyrazol-4-yl]-6-[8-([1,3]thiazolo[4,5-b]pyridin-2-ylcarbamoyl)naphthalen-2-yl]pyridine-2-carboxylic acid;
6-[8-(1,3-benzothiazol-2-ylcarbamoyl)-5-methoxy-3,4-dihydroisoquinolin-2(1H)-yl]-3-[1-(({3,5-dimethyl-7-[2-(methylamino)ethoxy]tricyclo[3.3.1.13,7]dec-1-yl}methyl)-5-methyl-1H-pyrazol-4-yl]pyridine-2-carboxylic acid;
6-[5-(1,3-benzothiazol-2-ylcarbamoyl)quinolin-3-yl]-3-[1-({3,5-dimethyl-7-[2-(methylamino)ethoxy]tricyclo[3.3.1.13,7]dec-1-yl}methyl)-5-methyl-1H-pyrazol-4-yl]pyridine-2-carboxylic acid;
6-[4-(1,3-benzothiazol-2-ylcarbamoyl)quinolin-6-yl]-3-[1-({3,5-dimethyl-7-[2-(methylamino)ethoxy]tricyclo[3.3.1.13,7]dec-1-yl}methyl)-5-methyl-1H-pyrazol-4-yl]pyridine-2-carboxylic acid;
6-[8-(1,3-benzothiazol-2-ylcarbamoyl)-5-methoxy-3,4-dihydroisoquinolin-2(1H)-yl]-3-{1-[(3-{2-[(2-methoxyethyl)amino]ethoxy}-5,7-dimethyltricyclo[3.3.1.13,7]dec-1-yl)methyl]-5-methyl-1H-pyrazol-4-yl}pyridine-2-carboxylic acid;
3-(1-{[3-(2-aminoethoxy)-5,7-dimethyltricyclo[3.3.1.13,7]dec-1-yl]methyl}-5-methyl-1H-pyrazol-4-yl)-6-[8-(1,3-benzothiazol-2-ylcarbamoyl)-5-cyano-3,4-dihydroisoquinolin-2(1H)-yl]pyridine-2-carboxylic acid;
6-[1-(1,3-benzothiazol-2-ylcarbamoyl)-1,2,3,4-tetrahydroquinolin-7-yl]-3-{1-[(3-{2-[(2-methoxyethyl)amino]ethoxy}-5,7-dimethyltricyclo[3.3.1.13,7]dec-1-yl)methyl]-5-methyl-1H-pyrazol-4-yl}pyridine-2-carboxylic acid;
6-[8-(1,3-benzothiazol-2-ylcarbamoyl)naphthalen-2-yl]-3-{1-[(3-{2-[(2-methoxyethyl)amino]ethoxy}-5,7-dimethyltricyclo[3.3.1.13,7]dec-1-yl)methyl]-5-methyl-1H-pyrazol-4-yl}pyridine-2-carboxylic acid;
6-[8-(1,3-benzothiazol-2-ylcarbamoyl)-3,4-dihydroisoquinolin-2(1H)-yl]-3-[1-({3,5-dimethyl-7-[2-(oxetan-3-ylamino)ethoxy]tricyclo[3.3.1.13,7]dec-1-yl}methyl)-5-methyl-1H-pyrazol-4-yl]pyridine-2-carboxylic acid;
6-[6-(3-aminopyrrolidin-1-yl)-8-(1,3-benzothiazol-2-ylcarbamoyl)-3,4-dihydroisoquinolin-2(1H)-yl]-3-(1-{[3-(2-methoxyethoxy)-5,7-dimethyltricyclo[3.3.1.1^{3,7}]dec-1-yl]methyl}-5-methyl-1H-pyrazol-4-yl)pyridine-2-carboxylic acid;
6-[8-(1,3-benzothiazol-2-ylcarbamoyl)-3,4-dihydroisoquinolin-2(1H)-yl]-3-{1-[(3,5-dimethyl-7-{2-[(2-sulfamoylethyl)amino]ethoxy}tricyclo[3.3.1.13,7]dec-1-yl)methyl]-5-methyl-1H-pyrazol-4-yl}pyridine-2-carboxylic acid;
3-(1-{[3-(2-aminoethoxy)-5,7-dimethyltricyclo[3.3.1.13,7]dec-1-yl]methyl}-5-methyl-1H-pyrazol-4-yl)-6-[3-(1,3-benzothiazol-2-ylcarbamoyl)-6,7-dihydrothieno[3,2-c]pyridin-5(4H)-yl]pyridine-2-carboxylic acid;
3-(1-{[3-(2-aminoethoxy)-5,7-dimethyltricyclo[3 0.3.1.1 3,7]dec-1-yl]methyl}-5-methyl-1H-pyrazol-4-yl)-6-[1-(1,3-benzothiazol-2-ylcarbamoyl)-3-(trifluoromethyl)-5,6-dihydroimidazo[1,5-a]pyrazin-7(8H)-yl]pyridine-2-carboxylic acid;
6-[8-(1,3-benzothiazol-2-ylcarbamoyl)-6-{methyl[2-(methylamino)ethyl]amino}-3,4-dihydroisoquinolin-2(1H)-yl]-3-(1-{[3-(2-methoxyethoxy)-5,7-dimethyltricyclo[3 0.3.1.1 3,7]dec-1-yl]methyl}-5-methyl-1H-pyrazol-4-yl)pyridine-2-carboxylic acid;
6-[8-(1,3-benzothiazol-2-ylcarbamoyl)-6-methoxy-3,4-dihydroisoquinolin-2(1H)-yl]-3-[1-({3,5-dimethyl-7-[2-(methylamino)ethoxy]tricyclo[3 0.3.1.1 3,7]dec-1l-yl}methyl)-5-methyl-1H-pyrazol-4-yl]pyridine-2-carboxylic acid;
3-(1-{[3-(2-aminoethoxy)-5,7-dimethyltricyclo[3 0.3.1.1 3,7]dec-1-yl]methyl}-5-methyl-1H-pyrazol-4-yl)-6-[4-(1,3-benzothiazol-2-ylcarbamoyl)quinolin-6-yl]pyridine-2-carboxylic acid;
6-[5-amino-8-(1,3-benzothiazol-2-ylcarbamoyl)-3,4-dihydroisoquinolin-2(1H)-yl]-3-[1-({3,5-dimethyl-7-[2-(methylamino)ethoxy]tricyclo[3 0.3.1.1 3,7]dec-1l-yl}methyl)-5-methyl-1H-pyrazol-4-yl]pyridine-2-carboxylic acid;
6-[8-(1,3-benzothiazol-2-ylcarbamoyl)-6-[3-(methylamino)prop-1-yn-1l-yl]-3,4-dihydroisoquinolin-2(1H)-yl]-3-(1-{[3-(2-methoxyethoxy)-5,7-dimethyltricyclo[3 0.3.1.1 3,7]dec-1-yl]methyl}-5-methyl-1H-pyrazol-4-yl)pyridine-2-carboxylic acid;
6-[4-(1,3-benzothiazol-2-ylcarbamoyl)isoquinolin-6-yl]-3-[1-({3,5-dimethyl-7-[2-(methylamino)ethoxy]tricyclo[3 0.3.1.1 3,7]dec-1-yl}methyl)-5-methyl-1H-pyrazol-4-yl]pyridine-2-carboxylic acid;
6-[7-(1,3-benzothiazol-2-ylcarbamoyl)-1H-indol-2-yl]-3-[1-({3,5-dimethyl-7-[2-(methylamino)ethoxy]tricyclo[3 0.3.1.1 3,7]dec-1-yl}methyl)-5-methyl-1H-pyrazol-4-yl]pyridine-2-carboxylic acid;
3-(1-{[3-(2-aminoethoxy)-5,7-dimethyltricyclo[3 0.3.1.1 3,7]dec-1-yl]methyl}-5-methyl-1H-pyrazol-4-yl)-6-[7-(1,3-benzothiazol-2-ylcarbamoyl)-1H-indol-2-yl]pyridine-2-carboxylic acid;
6-[7-(1,3-benzothiazol-2-ylcarbamoyl)-3-methyl-1H-indol-2-yl]-3-[1-({3,5-dimethyl-7-[2-(methylamino)ethoxy]tricyclo[3 0.3.1.1 3,7]dec-1-yl}methyl)-5-methyl-1H-pyrazol-4-yl]pyridine-2-carboxylic acid;
6-[8-(1,3-benzothiazol-2-ylcarbamoyl)-3,4-dihydroisoquinolin-2(1H)-yl]-3-(1-{[3,5-dimethyl-7-(2-{[1-(methylsulfonyl)piperidin-4-yl]amino}ethoxy)tricyclo[3.3.1.13,7]dec-1-yl]methyl}-5-methyl-1H-pyrazol-4-yl)pyridine-2-carboxylic acid;
6-[8-(1,3-benzothiazol-2-ylcarbamoyl)-3,4-dihydroisoquinolin-2(1H)-yl]-3-(1-{[3,5-dimethyl-7-(2-{[-(methylsulfonyl)azetidin-3-yl]amino}ethoxy)tricyclo[3.3.1.13,7]dec-1-yl]methyl}-5-methyl-1H-pyrazol-4-yl)pyridine-2-carboxylic acid;
3-{1-[(3-{2-[(3-amino-3-oxopropyl)amino]ethoxy}-5,7-dimethyltricyclo[3.3.1.13,7]dec-1-yl)methyl]-5-methyl-1H-pyrazol-4-yl}-6-[8-(1,3-benzothiazol-2-ylcarbamoyl)-3,4-dihydroisoquinolin-2(1H)-yl]pyridine-2-carboxylic acid;
6-[3-(1,3-benzothiazol-2-ylcarbamoyl)-1H-indazol-5-yl]-3-[1-({3,5-dimethyl-7-[2-(methylamino)ethoxy]tricyclo[3.3.1.13,7]dec-1-yl}methyl)-5-methyl-1H-pyrazol-4-yl]pyridine-2-carboxylic acid;
6-[3-(1,3-benzothiazol-2-ylcarbamoyl)-1H-indol-5-yl]-3-[1-({3,5-dimethyl-7-[2-(methylamino)ethoxy]tricyclo[3.3.1.13,7]dec-1-yl}methyl)-5-methyl-1H-pyrazol-4-yl]pyridine-2-carboxylic acid;
6-[3-(1,3-benzothiazol-2-ylcarbamoyl)-1H-pyrrolo[2,3-b]pyridin-5-yl]-3-[1-({3,5-dimethyl-7-[2-(methylamino)ethoxy]tricyclo[3.3.1.13,7]dec-1-yl}methyl)-5-methyl-1H-pyrazol-4-yl]pyridine-2-carboxylic acid;
6-(8-(benzo[d]thiazol-2-ylcarbamoyl)-3,4-dihydroisoquinolin-2(1H)-yl)-3-(1-((3-(2-((2-(N,N-dimethylsulfamoyl)ethyl)amino)ethoxy)-5,7-dimethyladamantan-1-yl)methyl)-5-methyl-1H-pyrazol-4-yl)picolinic acid;
6-[8-(1,3-benzothiazol-2-ylcarbamoyl)naphthalen-2-yl]-3-{1-[(3-{2-[(3-hydroxypropyl)amino]ethoxy}-5,7-dimethyltricyclo[3.3.1.13,7]dec-1-yl)methyl]-5-methyl-1H-pyrazol-4-yl}pyridine-2-carboxylic acid;
6-[8-(1,3-benzothiazol-2-ylcarbamoyl)-3,4-dihydroisoquinolin-2(1H)-yl]-3-(1-{[3-(2-{[3-(dimethylamino)-3-oxopropyl]amino}ethoxy)-5,7-dimethyltricyclo[3.3.1.13,7]dec-1-yl]methyl}-5-methyl-1H-pyrazol-4-yl)pyridine-2-carboxylic acid;
6-[8-(1,3-benzothiazol-2-ylcarbamoyl)-3,4-dihydroisoquinolin-2(1H)-yl]-3-(1-{[3,5-dimethyl-7-(2-{[3-(methylamino)-3-oxopropyl]amino}ethoxy)tricyclo[3.3.1.13,7]dec-1-yl]methyl}-5-methyl-1H-pyrazol-4-yl)pyridine-2-carboxylic acid;
3-(1-{[3-(2-aminoacetamido)-5,7-dimethyltricyclo[3.3.1.13,7]decan-1-yl]methyl}-5-methyl-1H-pyrazol-4-yl)-6-{8-[(1,3-benzothiazol-2-yl)carbamoyl]-3,4-dihydroisoquinolin-2(1H)-yl}pyridine-2-carboxylic acid;
3-[1-({3-[(2-aminoethyl)sulfanyl]-5,7-dimethyltricyclo[3.3.1.13,7]dec-1-yl}methyl)-5-methyl-1H-pyrazol-4-yl]-6-[8-(1,3-benzothiazol-2-ylcarbamoyl)-3,4-dihydroisoquinolin-2(1H)-yl]pyridine-2-carboxylic acid;
3-(1-{[3-(3-aminopropyl)-5,7-dimethyltricyclo[3.3.1.13,7]dec-1-yl]methyl}-5-methyl-1H-pyrazol-4-yl)-6-[8-(1,3-benzothiazol-2-ylcarbamoyl)-3,4-dihydroisoquinolin-2(1H)-yl]pyridine-2-carboxylic acid; and
3-(1-{[3-(2-aminoethoxy)-5,7-dimethyltricyclo[3.3.1.13,7]decan-1-yl]methyl}-5-methyl-1H-pyrazol-4-yl)-6-5-[(1,3-benzothiazol-2-yl)carbamoyl]quinolin-3-yl)pyridine-2-carboxylic acid.

12. The ADC of claim 9, wherein the anti-hCD98 antibody comprises a heavy chain variable region comprising the amino acid sequence set forth in SEQ ID NO: 108, SEQ ID NO: 110, SEQ ID NO: 115, or SEQ ID NO: 118, and a light chain variable region comprising the amino acid sequence set forth in SEQ ID NO: 107, SEQ ID NO: 112, or SEQ ID NO: 117.

13. The ADC of claim 9, wherein the anti-hCD98 antibody is selected from the group consisting of
an anti-hCD98 antibody comprising a heavy chain CDR3 domain comprising the amino acid sequence set forth in SEQ ID NO: 17, a heavy chain CDR2 domain comprising the amino acid sequence set forth in SEQ ID NO: 87, and a heavy chain CDR1 domain comprising the amino acid sequence set forth in SEQ ID NO: 16; a light chain CDR3 domain comprising the amino acid sequence set forth in SEQ ID NO: 19, a light chain CDR2 domain comprising the amino acid sequence set forth in SEQ ID NO: 7, and a light chain CDR1 domain comprising the amino acid sequence set forth in SEQ ID NO: 13;
an anti-hCD98 antibody comprising a heavy chain CDR3 domain comprising the amino acid sequence set forth in SEQ ID NO: 17, a heavy chain CDR2 domain comprising the amino acid sequence set forth in SEQ ID NO: 90, and a heavy chain CDR1 domain comprising the amino acid sequence set forth in SEQ ID NO: 16; a light chain CDR3 domain comprising the amino acid sequence set forth in SEQ ID NO: 19, a light chain CDR2 domain comprising the amino acid sequence set forth in SEQ ID NO: 7, and a light chain CDR1 domain comprising the amino acid sequence set forth in SEQ ID NO: 13;
an anti-hCD98 antibody comprising a heavy chain CDR3 domain comprising the amino acid sequence set forth in SEQ ID NO: 97, a heavy chain CDR2 domain comprising the amino acid sequence set forth in SEQ ID NO: 92, and a heavy chain CDR1 domain comprising the amino acid sequence set forth in SEQ ID NO: 79; a light chain CDR3 domain comprising the amino acid sequence set forth in SEQ ID NO: 95, a light chain CDR2 domain comprising the amino acid sequence set forth in SEQ ID NO: 45, and a light chain CDR1 domain comprising the amino acid sequence set forth in SEQ ID NO: 83; and
an anti-hCD98 antibody comprising a heavy chain CDR3 domain comprising the amino acid sequence set forth in SEQ ID NO: 97, a heavy chain CDR2 domain comprising the amino acid sequence set forth in SEQ ID NO: 104, and a heavy chain CDR1 domain comprising the amino acid sequence set forth in SEQ ID NO: 79; a light chain CDR3 domain comprising the amino acid sequence set forth in SEQ ID NO: 102, a light chain CDR2 domain comprising the amino acid sequence set forth in SEQ ID NO: 45, and a light chain CDR1 domain comprising the amino acid sequence set forth in SEQ ID NO: 83.

14. A pharmaceutical composition comprising an effective amount of an ADC according to claim 9, and a pharmaceutically acceptable carrier.

15. A pharmaceutical composition comprising an ADC mixture comprising a plurality of the ADC of claim 9, and a pharmaceutically acceptable carrier.

16. A method for treating cancer, comprising administering a therapeutically effective amount of the ADC of claim 9 to a subject in need thereof.

17. A method for inhibiting or decreasing solid tumor growth in a subject having a solid tumor, said method comprising administering an effective amount of the ADC of claim 9 to the subject having the solid tumor, such that the solid tumor growth is inhibited or decreased.

18. The method of claim 16, wherein the ADC is administered in combination with an additional agent or an additional therapy.

19. A process for the preparation of an ADC according to claim 10, wherein
the CD98 antibody comprises the heavy and light chain CDRs of huAb102, huAb014, huAb108, or huAb110;
the process comprising:
treating an antibody in an aqueous solution with an effective amount of a disulfide reducing agent at 30-40° C. for at least 15 minutes, and then cooling the antibody solution to 20-27° C.;
adding to the reduced antibody solution a solution of water/dimethyl sulfoxide comprising a synthon selected from the group of 2.1 to 2.31 and 2.34 to 2.72 (Table 5);
adjusting the pH of the solution to a pH of 7.5 to 8.5;
allowing the reaction to run for 48 to 80 hours to form the ADC;
wherein the mass is shifted by 18±2 amu for each hydrolysis of a succinimide to a succinamide as measured by electron spray mass spectrometry; and
wherein the ADC is optionally purified by hydrophobic interaction chromatography.

20. An anti-CD98 antibody drug conjugate (ADC) selected from the group consisting of formulae (i) or (ii): 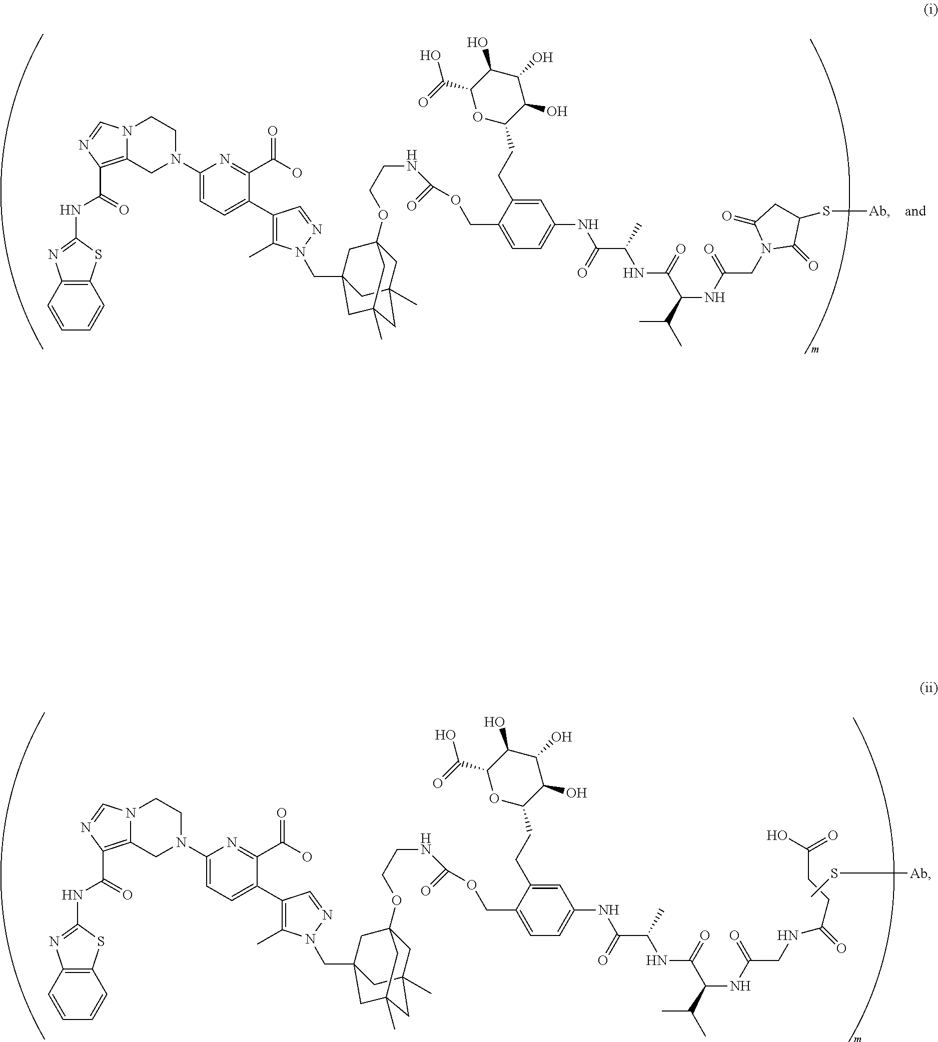
wherein m is an integer from 1 to 6, optionally from 2 to 6; and
wherein Ab is an anti-CD98 antibody comprising a heavy chain variable region and a light chain variable region selected from the group consisting of
a) a heavy chain CDR3 domain comprising the amino acid sequence set forth in SEQ ID NO: 17, a heavy chain CDR2 domain comprising the amino acid sequence set forth in SEQ ID NO: 87, and a heavy chain CDR1 domain comprising the amino acid sequence set forth in SEQ ID NO: 16; a light chain CDR3 domain comprising the amino acid sequence set forth in SEQ ID NO: 19, a light chain CDR2 domain comprising the amino acid sequence set forth in SEQ ID NO: 7, and a light chain CDR1 domain comprising the amino acid sequence set forth in SEQ ID NO: 13;
b) a heavy chain variable region comprising the amino acid sequence set forth in SEQ ID NO: 108, and a light chain variable region comprising the amino acid sequence set forth in SEQ ID NO: 107;
c) a heavy chain CDR3 domain comprising the amino acid sequence set forth in SEQ ID NO: 17, a heavy chain CDR2 domain comprising the amino acid sequence set forth in SEQ ID NO: 90, and a heavy chain CDR1 domain comprising the amino acid sequence set forth in SEQ ID NO: 16; a light chain CDR3 domain comprising the amino acid sequence set forth in SEQ ID NO: 19, a light chain CDR2 domain comprising the amino acid sequence set forth in SEQ ID NO: 7, and a light chain CDR1 domain comprising the amino acid sequence set forth in SEQ ID NO: 13;
d) a heavy chain variable region comprising the amino acid sequence set forth in SEQ ID NO: 110, and a light chain variable region comprising the amino acid sequence set forth in SEQ ID NO: 107;
e) a heavy chain CDR3 domain comprising the amino acid sequence set forth in SEQ ID NO: 97, a heavy chain CDR2 domain comprising the amino acid sequence set forth in SEQ ID NO: 92, and a heavy chain CDR1 domain comprising the amino acid sequence set forth in SEQ ID NO: 79; a light chain CDR3 domain comprising the amino acid sequence set forth in SEQ ID NO: 95, a light chain CDR2 domain comprising the amino acid sequence set forth in SEQ ID NO: 45, and a light chain CDR1 domain comprising the amino acid sequence set forth in SEQ ID NO: 83;
f) a heavy chain variable region comprising the amino acid sequence set forth in SEQ ID NO: 115, and a light chain variable region comprising the amino acid sequence set forth in SEQ ID NO: 112;
g) a heavy chain CDR3 domain comprising the amino acid sequence set forth in SEQ ID NO: 97, a heavy chain CDR2 domain comprising the amino acid sequence set forth in SEQ ID NO: 104, and a heavy chain CDR1 domain comprising the amino acid sequence set forth in SEQ ID NO: 79; a light chain CDR3 domain comprising the amino acid sequence set forth in SEQ ID NO: 102, a light chain CDR2 domain comprising the amino acid sequence set forth in SEQ ID NO: 45, and a light chain CDR1 domain comprising the amino acid sequence set forth in SEQ ID NO: 83; and
h) a heavy chain variable region comprising the amino acid sequence set forth in SEQ ID NO: 118, and a light chain variable region comprising the amino acid sequence set forth in SEQ ID NO: 117.


SEQUENCE LISTING (167 sequences)

SEQ ID NO: 1 - protein, 123 aa, Artificial Sequence
  source: /note=“Description of Artificial Sequence Synthetic polypeptide”
EVKLVESGGGLVQPGGSLRLSCATSGFTFTDYYMSWVRQPPGKALEWLGFIRNPANVYTTEYSASVKGRFTISRDNSQSILYLQMNTLRAEDSATYYCARASYGNSEGWFAYWGQGTLVTVSA

SEQ ID NO: 2 - protein, 10 aa, Artificial Sequence
  source: /note=“Description of Artificial Sequence Synthetic peptide”
GFTFTDYYMS

SEQ ID NO: 3 - protein, 19 aa, Artificial Sequence
  source: /note=“Description of Artificial Sequence Synthetic peptide”
FIRNPANVYTTEYSASVKG

SEQ ID NO: 4 - protein, 12 aa, Artificial Sequence
  source: /note=“Description of Artificial Sequence Synthetic peptide”
ASYGNSEGWFAY

SEQ ID NO: 5 - protein, 113 aa, Artificial Sequence
  source: /note=“Description of Artificial Sequence Synthetic polypeptide”
DIVMSQSPSSLAVSVGEKVTMSCKSSQNLLYNNNQKNYLAWYQQKPGQSPKLLIYWASTRESGVPDRFTGSGSGTDFTLTISSVKAEDLAVYYCQQYYSYPRTFGGGTKLEIK

SEQ ID NO: 6 - protein, 17 aa, Artificial Sequence
  source: /note=“Description of Artificial Sequence Synthetic peptide”
KSSQNLLYNNNQKNYLA

SEQ ID NO: 7 - protein, 7 aa, Artificial Sequence
  source: /note=“Description of Artificial Sequence Synthetic peptide”
WASTRES

SEQ ID NO: 8 - protein, 9 aa, Artificial Sequence
  source: /note=“Description of Artificial Sequence Synthetic peptide”
QQYYSYPRT

SEQ ID NO: 9 - protein, 123 aa, Artificial Sequence
  source: /note=“Description of Artificial Sequence Synthetic polypeptide”
EVKLVESGGGLVQPGGSLRLSCATSGFNFTDYYMSWVRQPPGKALEWLGFIRNKANGYTTEYSASVKGRFTISRDDSQSILYLQMNTLRAEDSATYYCARASYGNSEGWFAYWGQGTLVTVSA

SEQ ID NO: 10 - protein, 10 aa, Artificial Sequence
  source: /note=“Description of Artificial Sequence Synthetic peptide”
GFNFTDYYMS

SEQ ID NO: 11 - protein, 19 aa, Artificial Sequence
  source: /note=“Description of Artificial Sequence Synthetic peptide”
FIRNKANGYTTEYSASVKG

SEQ ID NO: 12 - protein, 113 aa, Artificial Sequence
  source: /note=“Description of Artificial Sequence Synthetic polypeptide”
DIVMSQSPSSLAVSVGEKVTMNCKSSQSLLYSSNQKNYLAWYQQKPGQSPKLLIYWASTRESGVPDRFTGSGSGTDFTLTISSVKAEDLAVYYCQQYYRYPRTFGGGTKLEIK

SEQ ID NO: 13 - protein, 17 aa, Artificial Sequence
  source: /note=“Description of Artificial Sequence Synthetic peptide”
KSSQSLLYSSNQKNYLA

SEQ ID NO: 14 - protein, 9 aa, Artificial Sequence
  source: /note=“Description of Artificial Sequence Synthetic peptide”
QQYYRYPRT

SEQ ID NO: 15 - protein, 119 aa, Artificial Sequence
  source: /note=“Description of Artificial Sequence Synthetic polypeptide”
EVKLVESGGGLVQPGNSLRLSCATSGFTFIDYYMSWVRQSPGKALEWLGFIRNKANGYTTEYSASVKGRFTISRDNSQSILYLQMDTLRAEDSATYYCTRDRPAWFVYWGQGTLVTVSA

SEQ ID NO: 16 - protein, 10 aa, Artificial Sequence
  source: /note=“Description of Artificial Sequence Synthetic peptide”
GFTFIDYYMS

SEQ ID NO: 17 - protein, 8 aa, Artificial Sequence
  source: /note=“Description of Artificial Sequence Synthetic peptide”
DRPAWFVY

SEQ ID NO: 18 - protein, 113 aa, Artificial Sequence
  source: /note=“Description of Artificial Sequence Synthetic polypeptide”
DIVMSQSPSSLAVSVGEKVTMSCKSSQSLLYSSNQKNYLAWYQQKPGQSPKLLIYWASTRESGVPDRFTGSGSGTDFTLTFSSVRAEDLAVYYCQQYYSYPYTFGGGTKLEIK

SEQ ID NO: 19 - protein, 9 aa, Artificial Sequence
  source: /note=“Description of Artificial Sequence Synthetic peptide”
QQYYSYPYT

SEQ ID NO: 20 - protein, 123 aa, Artificial Sequence
  source: /note=“Description of Artificial Sequence Synthetic polypeptide”
EVKLVESGGGLVQPGGSLRLSCTTSGFTFTDYYMSWVRQPPGKALEWLGFIRNKATIYTTEYSASVKGRFTISRDNSQSILYLQMNTLRAEDSATYYCARASYGNSEGWFAYWGQGTLVTVSA

SEQ ID NO: 21 - protein, 19 aa, Artificial Sequence
  source: /note=“Description of Artificial Sequence Synthetic peptide”
FIRNKATIYTTEYSASVKG

SEQ ID NO: 22 - protein, 113 aa, Artificial Sequence
  source: /note=“Description of Artificial Sequence Synthetic polypeptide”
DIVMSQSPSSLAVSVGEKVTMSCKSSQSLLYSSNQKNYLAWYQQKPGQSPKVLIYWASTRESGVPDRFTGSGSGTDFTLTISSVKAEDLAVYYCQQYYSYPRTFGGGTKLEIK

SEQ ID NO: 23 - protein, 123 aa, Artificial Sequence
  source: /note=“Description of Artificial Sequence Synthetic polypeptide”
EVKLVESGGGLVQPGGSLRLSCATSGFTFTDYYMTWVRQPPGKALEWLGFIRNKANGYTTEYSASVKGRFTISRDNSLSILYLQMNTLRAEDSATYYCARASYVNSEGWFAYWGQGTLVTVSA

SEQ ID NO: 24 - protein, 10 aa, Artificial Sequence
  source: /note=“Description of Artificial Sequence Synthetic peptide”
GFTFTDYYMT

SEQ ID NO: 25 - protein, 12 aa, Artificial Sequence
  source: /note=“Description of Artificial Sequence Synthetic peptide”
ASYVNSEGWFAY

SEQ ID NO: 26 - protein, 113 aa, Artificial Sequence
  source: /note=“Description of Artificial Sequence Synthetic polypeptide”
DIVMSQSPSSLAVSVGEKVTMSCKSSQSLLYSSNQKNYLAWYQQKLGQSPKLLIYWASTRESGVPDRFTGSGSGTDFTLTISSVKAEDLAVYYCQHYYSYPRTFGGGTKLEIK

SEQ ID NO: 27 - protein, 9 aa, Artificial Sequence
  source: /note=“Description of Artificial Sequence Synthetic peptide”
QHYYSYPRT

SEQ ID NO: 28 - protein, 122 aa, Artificial Sequence
  source: /note=“Description of Artificial Sequence Synthetic polypeptide”
QVQLKESGPGLAQPSQTLSLTCTVSGFSLSTYGVIWLRQPPGKGLEWMGVIWTNGNTNYNSTLKSRLSISRDTSESQVYLQMNSLQTEDTATYYCARHYYDGAYYYGYFDYWGQGVMVTVSS

SEQ ID NO: 29 - protein, 10 aa, Artificial Sequence
  source: /note=“Description of Artificial Sequence Synthetic peptide”
GFSLSTYGVI

SEQ ID NO: 30 - protein, 16 aa, Artificial Sequence
  source: /note=“Description of Artificial Sequence Synthetic peptide”
VIWTNGNTNYNSTLKS

SEQ ID NO: 31 - protein, 14 aa, Artificial Sequence
  source: /note=“Description of Artificial Sequence Synthetic peptide”
HYYDGAYYYGYFDY

SEQ ID NO: 32 - protein, 113 aa, Artificial Sequence
  source: /note=“Description of Artificial Sequence Synthetic polypeptide”
DIVMTQTPSSQAVSAGEKVTMSCKSSQSLLYSENKKNYLAWYQQKPGQSPKLLIYWASTRESGVPDRFIGSGSGTDFTLTISSVQAEDLAVYYCQQYYYFPYTFGAGTKLELK

SEQ ID NO: 33 - protein, 17 aa, Artificial Sequence
  source: /note=“Description of Artificial Sequence Synthetic peptide”
KSSQSLLYSENKKNYLA

SEQ ID NO: 34 - protein, 9 aa, Artificial Sequence
  source: /note=“Description of Artificial Sequence Synthetic peptide”
QQYYYFPYT

SEQ ID NO: 35 - protein, 122 aa, Artificial Sequence
  source: /note=“Description of Artificial Sequence Synthetic polypeptide”
QVQLKESGPGLVQPSQTLSLTCTVSGFSLSTYGVIWVRQPPGKGLEWMGVIWANGNTNYNSTLKSRLSISRDTSKSQVYLKMNSLQTEDTATYYCARHYYDGTYYYGYFDYWGQGVMVTVSS

SEQ ID NO: 36 - protein, 16 aa, Artificial Sequence
  source: /note=“Description of Artificial Sequence Synthetic peptide”
VIWANGNTNYNSTLKS

SEQ ID NO: 37 - protein, 14 aa, Artificial Sequence
  source: /note=“Description of Artificial Sequence Synthetic peptide”
HYYDGTYYYGYFDY

SEQ ID NO: 38 - protein, 113 aa, Artificial Sequence
  source: /note=“Description of Artificial Sequence Synthetic polypeptide”
DIVMTQTPSSQAVSAGEKVTMNCKSSQSLLYSENKKNYLAWYQQKPGQSPKLLIYWASTRESGVPDRFIGSGSGTDFTLTISSVQAEDLAVYYCQQYYYFPYTFGPGTKLELK

SEQ ID NO: 39 - protein, 123 aa, Artificial Sequence
  source: /note=“Description of Artificial Sequence Synthetic polypeptide”
EVQLVESGGGLVQPGRSLKLSCAASGFTFSDYAMAWVRQAPKKGLEWVASIIYDGRGTYYRDSVKGRFTISRDNAKSTLYLQMDSLRSEDTATYYCARQGDGTYYYWGYFDYWGQGVMVTVSS

SEQ ID NO: 40 - protein, 10 aa, Artificial Sequence
  source: /note=“Description of Artificial Sequence Synthetic peptide”
GFTFSDYAMA

SEQ ID NO: 41 - protein, 17 aa, Artificial Sequence
  source: /note=“Description of Artificial Sequence Synthetic peptide”
SIIYDGRGTYYRDSVKG

SEQ ID NO: 42 - protein, 14 aa, Artificial Sequence
  source: /note=“Description of Artificial Sequence Synthetic peptide”
QGDGTYYYWGYFDY

SEQ ID NO: 43 - protein, 113 aa, Artificial Sequence
  source: /note=“Description of Artificial Sequence Synthetic polypeptide”
DIVMTQSPSSLAVSAGETVTINCKSSQSLLSSGNQKNYLAWYQQKPGQSPKLLIYWASTRQSGVPDRFIGSGSGTDFTLTINSVQAEDLAIYYCQQYYDTPYTFGAGTKLELK

SEQ ID NO: 44 - protein, 17 aa, Artificial Sequence
  source: /note=“Description of Artificial Sequence Synthetic peptide”
KSSQSLLSSGNQKNYLA

SEQ ID NO: 45 - protein, 7 aa, Artificial Sequence
  source: /note=“Description of Artificial Sequence Synthetic peptide”
WASTRQS

SEQ ID NO: 46 - protein, 9 aa, Artificial Sequence
  source: /note=“Description of Artificial Sequence Synthetic peptide”
QQYYDTPYT

SEQ ID NO: 47 - protein, 122 aa, Artificial Sequence
  source: /note=“Description of Artificial Sequence Synthetic polypeptide”
QVQLKESGPGLVQPSQTLSLTCAVSGFSLSNYGVIWVRQPPGKGLEWMAVIWTNGNTNYNSTLKSRLSISRDTSKSQVYLKMNSLQTEDTATYYCARHYYDGTYYYGYFDYWGQGVMVTVSS

SEQ ID NO: 48 - protein, 10 aa, Artificial Sequence
  source: /note=“Description of Artificial Sequence Synthetic peptide”
GFSLSNYGVI

SEQ ID NO: 49 - protein, 113 aa, Artificial Sequence
  source: /note=“Description of Artificial Sequence Synthetic polypeptide”
DIVMTQTPSSQAVSAGEKVTMSCKSSQSLLYTENKKNYLAWYQQKPGQSPKLLIYWASTRESGVPDRFMGSGSGTDFTLTISSVQAEDLAVYYCQQYYYFPYMFGAGTKLELK

SEQ ID NO: 50 - protein, 17 aa, Artificial Sequence
  source: /note=“Description of Artificial Sequence Synthetic peptide”
KSSQSLLYTENKKNYLA

SEQ ID NO: 51 - protein, 9 aa, Artificial Sequence
  source: /note=“Description of Artificial Sequence Synthetic peptide”
QQYYYFPYM

SEQ ID NO: 52 - protein, 123 aa, Artificial Sequence
  source: /note=“Description of Artificial Sequence Synthetic polypeptide”
EVQLVESGGGLVQPGRSLKLSCAASGFTFSDYAMAWVRQAPKKSLEWVATIIYDGRGTYCRDSVKGRFTISRDNAKSTLYLQMDSLRSEDTATYYCARQGDGTYHYWGYFDYWGQGVMVTVSS

SEQ ID NO: 53 - protein, 17 aa, Artificial Sequence
  source: /note=“Description of Artificial Sequence Synthetic peptide”
TIIYDGRGTYCRDSVKG

SEQ ID NO: 54 - protein, 14 aa, Artificial Sequence
  source: /note=“Description of Artificial Sequence Synthetic peptide”
QGDGTYHYWGYFDY

SEQ ID NO: 55 - protein, 113 aa, Artificial Sequence
  source: /note=“Description of Artificial Sequence Synthetic polypeptide”
DIVMTQSPSSLAVSAGETVTINCKSSQSLLSSGNQKNYLAWYQQKPGQSPKLLIYWASTRQSGVPDRFIGSGSGTDFTLTISSVQAEDLAIYYCQQYYDTPYTFGAGTKVDLK

SEQ ID NO: 56 - protein, 123 aa, Artificial Sequence
  source: /note=“Description of Artificial Sequence Synthetic polypeptide”
EVQLVESGGGLVQPGRSLKLSCAASGFTFSDYAMAWVRQAPKKGLEWVAGIIYDGRGTYYRDSVKGRFTISRDNAKSTLYLQMDSLRSEDTATYYCARQGDGTYYYWGYFDYWGQGVMVTVSS

SEQ ID NO: 57 - protein, 17 aa, Artificial Sequence
  source: /note=“Description of Artificial Sequence Synthetic peptide”
GIIYDGRGTYYRDSVKG

SEQ ID NO: 58 - protein, 113 aa, Artificial Sequence
  source: /note=“Description of Artificial Sequence Synthetic polypeptide”
DIVMTQSPSSLAVSAGETVTINCRSSQSLLSSGNQKNYLAWYQQKPGQSPKLLIYWASTRQSGVPDRFIGSGSGTDFTLTISSVQAEDLAIYYCQQYYDTPYTFGAGTKLELK

SEQ ID NO: 59 - protein, 17 aa, Artificial Sequence
  source: /note=“Description of Artificial Sequence Synthetic peptide”
RSSQSLLSSGNQKNYLA

SEQ ID NO: 60 - protein, 123 aa, Artificial Sequence
  source: /note=“Description of Artificial Sequence Synthetic polypeptide”
EVQLVESGGGLVQPGRSLKLSCAASGFTFSDYAMAWVRQAPKKGLEWVASIIYDGRGTYYRDSVKGRFTISRDNAKSTLYLQMDSLRSEDTATYYCARQGDGTYYYWGSFDYWGQGVMVTVSS

SEQ ID NO: 61 - protein, 14 aa, Artificial Sequence
  source: /note=“Description of Artificial Sequence Synthetic peptide”
QGDGTYYYWGSFDY

SEQ ID NO: 62 - protein, 113 aa, Artificial Sequence
  source: /note=“Description of Artificial Sequence Synthetic polypeptide”
DIVMTQSPSSLAVSAGETVTINCKSSQSLLSSGNQKNYLAWYQQKPGQSPKLLIYWASTRQSGVPDRFIGSGSGTDFTLTISSVQAEDLAIYHCQQYYDTPYTFGAGTKLELK

SEQ ID NO: 63 - protein, 122 aa, Artificial Sequence
  source: /note=“Description of Artificial Sequence Synthetic polypeptide”
QVQLKESGPGLVQPSQTLSLTCTVSGFSLSSYGVIWVRQPPGKGLEWMGIIWANGNTNYNSALKSRLSISRDTSKSQVYLKMNSLQTEDTATYYCARHYYDGTHYYGYFDYWGQGVMVTVSS

SEQ ID NO: 64 - protein, 10 aa, Artificial Sequence
  source: /note=“Description of Artificial Sequence Synthetic peptide”
GFSLSSYGVI

SEQ ID NO: 65 - protein, 16 aa, Artificial Sequence
  source: /note=“Description of Artificial Sequence Synthetic peptide”
IIWANGNTNYNSALKS

SEQ ID NO: 66 - protein, 14 aa, Artificial Sequence
  source: /note=“Description of Artificial Sequence Synthetic peptide”
HYYDGTHYYGYFDY

SEQ ID NO: 67 - protein, 113 aa, Artificial Sequence
  source: /note=“Description of Artificial Sequence Synthetic polypeptide”
DTVMTQTPSSQAVSAGEKVTMSCKSSQSLLYSENKKKYLAWYQQKPGQSPKLLIYWASTRESGVPDRFIGSGSGTDFTLTISSVQAEDLAVYYCQQYYNFPYTFGAGTKLELK

SEQ ID NO: 68 - protein, 17 aa, Artificial Sequence
  source: /note=“Description of Artificial Sequence Synthetic peptide”
KSSQSLLYSENKKKYLA

SEQ ID NO: 69 - protein, 9 aa, Artificial Sequence
  source: /note=“Description of Artificial Sequence Synthetic peptide”
QQYYNFPYT

SEQ ID NO: 70 - protein, 117 aa, Artificial Sequence
  source: /note=“Description of Artificial Sequence Synthetic polypeptide”
EVKLQQSGDELVRPGASVKISCKASGYTFTSYSMHWVKERPGQGLEWIGAIFPIIGTTEYNQKFKGKATLTADKSSNTANMELSRLTSEDSAVYYCARVYLSYFDYWGQGVMVTVSS

SEQ ID NO: 71 - protein, 10 aa, Artificial Sequence
  source: /note=“Description of Artificial Sequence Synthetic peptide”
GYTFTSYSMH

SEQ ID NO: 72 - protein, 17 aa, Artificial Sequence
  source: /note=“Description of Artificial Sequence Synthetic peptide”
AIFPIIGTTEYNQKFKG

SEQ ID NO: 73 - protein, 8 aa, Artificial Sequence
  source: /note=“Description of Artificial Sequence Synthetic peptide”
VYLSYFDY

SEQ ID NO: 74 - protein, 106 aa, Artificial Sequence
  source: /note=“Description of Artificial Sequence Synthetic polypeptide”
DIQMTQSPSFLSASVGDRVTINCKASQNINKYLDWYQRKHGEAPKLLIYNTNNLQTGIPSRFSGSGSGTDYTLTISSLQPEDVATYFCLQHSSRYTFGAGTKLELK

SEQ ID NO: 75 - protein, 11 aa, Artificial Sequence
  source: /note=“Description of Artificial Sequence Synthetic peptide”
KASQNINKYLD

SEQ ID NO: 76 - protein, 7 aa, Artificial Sequence
  source: /note=“Description of Artificial Sequence Synthetic peptide”
NTNNLQT

SEQ ID NO: 77 - protein, 8 aa, Artificial Sequence
  source: /note=“Description of Artificial Sequence Synthetic peptide”
LQHSSRYT

SEQ ID NO: 78 - protein, 123 aa, Artificial Sequence
  source: /note=“Description of Artificial Sequence Synthetic polypeptide”
EVQLVESGGGLVQPGRSLKLSCAASGFTFSDYTMAWVRQAPKKGLEWVATIIYDGRGTYYRDSVKGRFTISRDNAKSTLYLQMDSLRSEDTATYYCARQSDGTYYYWGYFDYWGQGVMVTVSS

SEQ ID NO: 79 - protein, 10 aa, Artificial Sequence
  source: /note=“Description of Artificial Sequence Synthetic peptide”
GFTFSDYTMA

SEQ ID NO: 80 - protein, 17 aa, Artificial Sequence
  source: /note=“Description of Artificial Sequence Synthetic peptide”
TIIYDGRGTYYRDSVKG

SEQ ID NO: 81 - protein, 14 aa, Artificial Sequence
  source: /note=“Description of Artificial Sequence Synthetic peptide”
QSDGTYYYWGYFDY

SEQ ID NO: 82 - protein, 113 aa, Artificial Sequence
  source: /note=“Description of Artificial Sequence Synthetic polypeptide”
DIVMTQSPSSLAVSAGETVTINCKSSQSLLFSGNQKNYLAWYQQKPGQSPKLLIYWASTRQSGVPDRFIGSGSGTDFTLTIRSVQAEDLAIYYCQQYYDSPYTFGAGTKLELK

SEQ ID NO: 83 - protein, 17 aa, Artificial Sequence
  source: /note=“Description of Artificial Sequence Synthetic peptide”
KSSQSLLFSGNQKNYLA

SEQ ID NO: 84 - protein, 9 aa, Artificial Sequence
  source: /note=“Description of Artificial Sequence Synthetic peptide”
QQYYDSPYT

SEQ ID NO: 85 - protein, 119 aa, Artificial Sequence
  source: /note=“Description of Artificial Sequence Synthetic polypeptide”
EVQLVESGGGLVQPGRSLRLSCTTSGFTFIDYYMSWVRQAPGKGLEWLGFIRNKANGYTTEYSASVKGRFTISRDNSKSILYLQMNSLKTEDTAVYYCTRDRPAWFVYWGQGTLVTVSS

SEQ ID NO: 86 - protein, 119 aa, Artificial Sequence
  source: /note=“Description of Artificial Sequence Synthetic polypeptide”
EVQLVESGGGLVQPGRSLRLSCTTSGFTFIDYYMSWVRQAPGKGLEWLGFIRNKANRYTTEYSASVKGRFTISRDNSKSILYLQMNSLKTEDTAVYYCTRDRPAWFVYWGQGTLVTVSS

SEQ ID NO: 87 - protein, 19 aa, Artificial Sequence
  source: /note=“Description of Artificial Sequence Synthetic peptide”
FIRNKANRYTTEYSASVKG

SEQ ID NO: 88 - protein, 113 aa, Artificial Sequence
  source: /note=“Description of Artificial Sequence Synthetic polypeptide”
DIVMTQSPDSLAVSLGERATINCKSSQSLLYSSNQKNYLAWYQQKPGQSPKLLIYWASTRESGVPDRFSGSGSGTDFTLTISSLQAEDVAVYYCQQYYSYPYTFGGGTKVEIK

SEQ ID NO: 89 - protein, 119 aa, Artificial Sequence
  source: /note=“Description of Artificial Sequence Synthetic polypeptide”
EVQLVESGGGLVQPGRSLRLSCTTSGFTFIDYYMSWVRQAPGKGLEWLGFIRNKAYGYTTEYSASVKGRFTISRDNSKSILYLQMNSLKTEDTAVYYCTRDRPAWFVYWGQGTLVTVSS

SEQ ID NO: 90 - protein, 19 aa, Artificial Sequence
  source: /note=“Description of Artificial Sequence Synthetic peptide”
FIRNKAYGYTTEYSASVKG

SEQ ID NO: 91 - protein, 123 aa, Artificial Sequence
  source: /note=“Description of Artificial Sequence Synthetic polypeptide”
EVQLVESGGGVVQPGRSLRLSCAASGFTFSDYTMAWVRQAPGKGLEWVATIIYSGRGTYYRDAVKGRFTISRDNSKSTLYLQMNSLRAEDTAVYYCARQSDHTYYYWGYFDYWGQGTMVTVSS

SEQ ID NO: 92 - protein, 17 aa, Artificial Sequence
  source: /note=“Description of Artificial Sequence Synthetic peptide”
TIIYSGRGTYYRDAVKG

SEQ ID NO: 93 - protein, 14 aa, Artificial Sequence
  source: /note=“Description of Artificial Sequence Synthetic peptide”
QSDHTYYYWGYFDY

SEQ ID NO: 94 - protein, 113 aa, Artificial Sequence
  source: /note=“Description of Artificial Sequence Synthetic polypeptide”
DIVMTQSPDSLAVSLGERATINCKSSQSLLFSGNQKNYLAWYQQKPGQSPKLLIYWASTRQSGVPDRFSGSGSGTDFTLTIRSLQAEDVAIYYCQQYYDVPYTFGQGTKLEIK

SEQ ID NO: 95 - protein, 9 aa, Artificial Sequence
  source: /note=“Description of Artificial Sequence Synthetic peptide”
QQYYDVPYT

SEQ ID NO: 96 - protein, 123 aa, Artificial Sequence
  source: /note=“Description of Artificial Sequence Synthetic polypeptide”
EVQLVESGGGVVQPGRSLRLSCAASGFTFSDYTMAWVRQAPGKGLEWVATIIYSGRGTYYRDAVKGRFTISRDNSKSTLYLQMNSLRAEDTAVYYCARQSDDTYYYWGYFDYWGQGTMVTVSS

SEQ ID NO: 97 - protein, 14 aa, Artificial Sequence
  source: /note=“Description of Artificial Sequence Synthetic peptide”
QSDDTYYYWGYFDY

SEQ ID NO: 98 - protein, 113 aa, Artificial Sequence
  source: /note=“Description of Artificial Sequence Synthetic polypeptide”
DIVMTQSPDSLAVSLGERATINCKSSQSLLFSGNQKNYLAWYQQKPGQSPKLLIYWASTRQSGVPDRFSGSGSGTDFTLTIRSLQAEDVAIYYCQQYYGSPYTFGQGTKLEIK

SEQ ID NO: 99 - protein, 123 aa, Artificial Sequence
  source: /note=“Description of Artificial Sequence Synthetic polypeptide”
EVQLVESGGGVVQPGRSLRLSCAASGFTFSDYTMAWVRQAPGKGLEWVATIIYTGRGTYYRDAVKGRFTISRDNSKSTLYLQMNSLRAEDTAVYYCARQSDDTYYYWGYFDYWGQGTMVTVSS

SEQ ID NO: 100 - protein, 17 aa, Artificial Sequence
  source: /note=“Description of Artificial Sequence Synthetic peptide”
TIIYTGRGTYYRDAVKG

SEQ ID NO: 101 - protein, 113 aa, Artificial Sequence
  source: /note=“Description of Artificial Sequence Synthetic polypeptide”
DIVMTQSPDSLAVSLGERATINCKSSQSLLFSGNQKNYLAWYQQKPGQSPKLLIYWASTRQSGVPDRFSGSGSGTDFTLTIRSLQAEDVAIYYCQQYYSSPYTFGQGTKLEIK

SEQ ID NO: 102 - protein, 9 aa, Artificial Sequence
  source: /note=“Description of Artificial Sequence Synthetic peptide”
QQYYSSPYT

SEQ ID NO: 103 - protein, 123 aa, Artificial Sequence
  source: /note=“Description of Artificial Sequence Synthetic polypeptide”
EVQLVESGGGVVQPGRSLRLSCAASGFTFSDYTMAWVRQAPGKGLEWVATIIYDARGTYYRDAVKGRFTISRDNSKSTLYLQMNSLRAEDTAVYYCARQSDDTYYYWGYFDYWGQGTMVTVSS

SEQ ID NO: 104 - protein, 17 aa, Artificial Sequence
  source: /note=“Description of Artificial Sequence Synthetic peptide”
TIIYDARGTYYRDAVKG

SEQ ID NO: 105 - protein, 9 aa, Artificial Sequence
  source: /note=“Description of Artificial Sequence Synthetic peptide”
QQYYGSPYT

SEQ ID NO: 106 - protein, 119 aa, Artificial Sequence
  source: /note=“Description of Artificial Sequence Synthetic polypeptide”
EVQLVESGGGLVKPGGSLRLSCATSGFTFIDYYMSWVRQAPGKGLEWLGFIRNKANRYTTEYSASVKGRFTISRDNSKSILYLQMNSLKTEDTAVYYCTRDRPAWFVYWGQGTLVTVSS

SEQ ID NO: 107 - protein, 113 aa, Artificial Sequence
  source: /note=“Description of Artificial Sequence Synthetic polypeptide”
DIVMTQTPLSLPVTPGEPASISCKSSQSLLYSSNQKNYLAWYLQKPGQSPQLLIYWASTRESGVPDRFSGSGSGTDFTLKISRVEAEDVGVYYCQQYYSYPYTFGGGTKVEIK

SEQ ID NO: 108 - protein, 119 aa, Artificial Sequence
  source: /note=“Description of Artificial Sequence Synthetic polypeptide”
EVQLVESGGGLVQPGGSLRLSCATSGFTFIDYYMSWVRQAPGKGLEWLGFIRNKANRYTTEYSASVKGRFTISRDNSKSILYLQMNSLKTEDTAVYYCTRDRPAWFVYWGQGTLVTVSS

SEQ ID NO: 109 - protein, 119 aa, Artificial Sequence
  source: /note=“Description of Artificial Sequence Synthetic polypeptide”
EVQLVESGGGLVKPGGSLRLSCATSGFTFIDYYMSWVRQAPGKGLEWLGFIRNKAYGYTTEYSASVKGRFTISRDNSKSILYLQMNSLKTEDTAVYYCTRDRPAWFVYWGQGTLVTVSS

SEQ ID NO: 110 - protein, 119 aa, Artificial Sequence
  source: /note=“Description of Artificial Sequence Synthetic polypeptide”
EVQLVESGGGLVQPGGSLRLSCATSGFTFIDYYMSWVRQAPGKGLEWLGFIRNKAYGYTTEYSASVKGRFTISRDNSKSILYLQMNSLKTEDTAVYYCTRDRPAWFVYWGQGTLVTVSS

SEQ ID NO: 111 - protein, 123 aa, Artificial Sequence
  source: /note=“Description of Artificial Sequence Synthetic polypeptide”
EVQLVESGGGLVQPGGSLRLSCAASGFTFSDYTMAWVRQAPGKGLEWVATIIYSGRGTYYRDAVKGRFTISRDNAKNTLYLQMNSLRAEDTAVYYCARQSDHTYYYWGYFDYWGQGTMVTVSS

SEQ ID NO: 112 - protein, 113 aa, Artificial Sequence
  source: /note=“Description of Artificial Sequence Synthetic polypeptide”
DIVMTQTPLSLPVTPGEPASINCKSSQSLLFSGNQKNYLAWYLQKPGQSPQLLIYWASTRQSGVPDRFSGSGSGTDFTLKISRVEAEDVGIYYCQQYYDVPYTFGQGTKLEIK

SEQ ID NO: 113 - protein, 123 aa, Artificial Sequence
  source: /note=“Description of Artificial Sequence Synthetic polypeptide”
EVQLVQSGAEVKKPGASVKVSCKASGFTFSDYTMAWVRQAPGQGLEWVATIIYSGRGTYYRDAVKGRFTITRDNSTSTLYLELSSLRSEDTAVYYCARQSDHTYYYWGYFDYWGQGTMVTVSS

SEQ ID NO: 114 - protein, 123 aa, Artificial Sequence
  source: /note=“Description of Artificial Sequence Synthetic polypeptide”
EVQLVESGGGLVQPGGSLRLSCAASGFTFSDYTMAWVRQAPGKGLEWVATIIYSGRGTYYRDAVKGRFTISRDNAKNTLYLQMNSLRAEDTAVYYCARQSDDTYYYWGYFDYWGQGTMVTVSS

SEQ ID NO: 115 - protein, 123 aa, Artificial Sequence
  source: /note=“Description of Artificial Sequence Synthetic polypeptide”
EVQLVQSGAEVKKPGASVKVSCKASGFTFSDYTMAWVRQAPGQGLEWVATIIYSGRGTYYRDAVKGRFTITRDNSTSTLYLELSSLRSEDTAVYYCARQSDDTYYYWGYFDYWGQGTMVTVSS

SEQ ID NO: 116 - protein, 123 aa, Artificial Sequence
  source: /note=“Description of Artificial Sequence Synthetic polypeptide”
EVQLVESGGGLVQPGGSLRLSCAASGFTFSDYTMAWVRQAPGKGLEWVATIIYDARGTYYRDAVKGRFTISRDNAKNTLYLQMNSLRAEDTAVYYCARQSDDTYYYWGYFDYWGQGTMVTVSS

SEQ ID NO: 117 - protein, 113 aa, Artificial Sequence
  source: /note=“Description of Artificial Sequence Synthetic polypeptide”
DIVMTQTPLSLPVTPGEPASINCKSSQSLLFSGNQKNYLAWYLQKPGQSPQLLIYWASTRQSGVPDRFSGSGSGTDFTLKISRVEAEDVGIYYCQQYYSSPYTFGQGTKLEIK

SEQ ID NO: 118 - protein, 123 aa, Artificial Sequence
  source: /note=“Description of Artificial Sequence Synthetic polypeptide”
EVQLVQSGAEVKKPGASVKVSCKASGFTFSDYTMAWVRQAPGQGLEWVATIIYDARGTYYRDAVKGRFTITRDNSTSTLYLELSSLRSEDTAVYYCARQSDDTYYYWGYFDYWGQGTMVTVSS

SEQ ID NO: 119 - protein, 123 aa, Artificial Sequence
  source: /note=“Description of Artificial Sequence Synthetic polypeptide”
EVQLVQSGAEVKKPGASVKVSCKASGFTFSDYTMAWVRQAPGQGLEWVATIIYSGRGTYYRDAVKGRFTITRDTSTSTLYLELSSLRSEDTAVYYCARQSDHTYYYWGYFDYWGQGTMVTVSS

SEQ ID NO: 120 - protein, 123 aa, Artificial Sequence
  source: /note=“Description of Artificial Sequence Synthetic polypeptide”
EVQLVQSGAEVKKPGASVKVSCKASGFTFSDYTMAWVRQAPGQGLEWVATIIYSGRGTYYRDAVKGRFTITRDTSTSTLYLELSSLRSEDTAVYYCARQSDDTYYYWGYFDYWGQGTMVTVSS

SEQ ID NO: 121 - protein, 123 aa, Artificial Sequence
  source: /note=“Description of Artificial Sequence Synthetic polypeptide”
EVQLVQSGAEVKKPGASVKVSCKASGFTFSDYTMAWVRQAPGQGLEWVATIIYDARGTYYRDAVKGRFTITRDTSTSTLYLELSSLRSEDTAVYYCARQSDDTYYYWGYFDYWGQGTMVTVSS

SEQ ID NO: 122 - protein, 123 aa, Artificial Sequence
  source: /note=“Description of Artificial Sequence Synthetic polypeptide”
EVQLVESGGGVVQPGRSLRLSCAASGFTFSDYTMAWVRQAPGKGLEWVATIIYDGRGTYYRDSVKGRFTISRDNSKSTLYLQMNSLRAEDTAVYYCARQSDGTYYYWGYFDYWGQGTMVTVSS

SEQ ID NO: 123 - protein, 113 aa, Artificial Sequence
  source: /note=“Description of Artificial Sequence Synthetic polypeptide”
DIVMTQSPDSLAVSLGERATINCKSSQSLLFSGNQKNYLAWYQQKPGQSPKLLIYWASTRQSGVPDRFSGSGSGTDFTLTIRSLQAEDVAIYYCQQYYDSPYTFGQGTKLEIK

SEQ ID NO: 124 - protein, 630 aa, Homo sapiens
MELQPPEASIAVVSIPRQLPGSHSEAGVQGLSAGDDSELGSHCVAQTGLELLASGDPLPSASQNAEMIETGSDCVTQAGLQLLASSDPPALASKNAEVTGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 125 - protein, 425 aa, Homo sapiens
RAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 126 - protein, 439 aa, Artificial Sequence
  source: /note=“Description of Artificial Sequence Synthetic polypeptide”
GGSGGHHHHHHRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAAAAA

SEQ ID NO: 127 - protein, 434 aa, Artificial Sequence
  source: /note=“Description of Artificial Sequence Synthetic polypeptide”
RAPRCRELPAQKWWHTGALYRIGDLQAFQGHGSGNLAGLKGRLDYLSSLKVKGLVLGPLHKNQKDDVAQTDLLQIDPNFGSKEDFDNLLQSAKKKSIRVILDLTPNYRGENLWFSTQVDSVATKVKDALEFWLQAGVDGFQVRDIENLKDASSFLAEWENITKGFSEDRLLIAGTNSSDLQQIVSLLESNKDLLLTSSYLSDSSFTGEHTKSLVTQYLNATGNRWCSWSLSQAGLLTSFLPAQLLRLYQLMLFTLPGTPVFSYGDEIGLKAAALPGQPVEAPVMLWDESSFPDIPGAVSANMTVKGQSEDPGSLLSLFRQLSDQRSKERSLLHGDFHTFSSGPGLFSYIRHWDQNERFLVVLNFGDVGLSAGLQASDLPASASLPTKADLVLSTQPGREEGSPLELERLKLEPHEGLLLRFPYVAAAAHHHHHH

SEQ ID NO: 128 - protein, 434 aa, Artificial Sequence
  source: /note=“Description of Artificial Sequence Synthetic polypeptide”
RAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAAAAAHHHHHH

SEQ ID NO: 129 - protein, 434 aa, Artificial Sequence; identical to SEQ ID NO: 127
  source: /note=“Description of Artificial Sequence Synthetic polypeptide”

SEQ ID NO: 130 - protein, 119 aa, Artificial Sequence
  source: /note=“Description of Artificial Sequence Synthetic polypeptide”
EVQLVESGGGLVQPGRSLRLSCTASGFTFIDYYMSWFRQAPGKGLEWVGFIRNKANGYTTEYSASVKGRFTISRDDSKSIAYLQMNSLKTEDTAVYYCTRDRPAWFVYWGQGTLVTVSS

SEQ ID NO: 131 - protein, 119 aa, Artificial Sequence
  source: /note=“Description of Artificial Sequence Synthetic polypeptide”
EVKLVESGGGLVQPGRSLRLSCTASGFTFIDYYMSWVRQAPGKGLEWLGFIRNKANGYTTEYSASVKGRFTISRDDSKSILYLQMNSLKTEDTAVYYCTRDRPAWFVYWGQGTLVTVSS

SEQ ID NO: 132 - protein, 119 aa, Artificial Sequence
  source: /note=“Description of Artificial Sequence Synthetic polypeptide”
EVKLVESGGGLVQPGRSLRLSCTASGFTFIDYYMSWFRQAPGKGLEWLGFIRNKANGYTTEYSASVKGRFTISRDDSKSIAYLQMNSLKTEDTAVYYCTRDRPAWFVYWGQGTLVTVSS

SEQ ID NO: 133 - protein, 119 aa, Artificial Sequence
  source: /note=“Description of Artificial Sequence Synthetic polypeptide”
EVQLVESGGGLVKPGGSLRLSCAASGFTFIDYYMSWVRQAPGKGLEWVGFIRNKANGYTTEYSASVKGRFTISRDDSKNTLYLQMNSLKTEDTAVYYCTTDRPAWFVYWGQGTLVTVSS

SEQ ID NO: 134 - protein, 119 aa, Artificial Sequence
  source: /note=“Description of Artificial Sequence Synthetic polypeptide”
EVQLVESGGGLVKPGGSLRLSCATSGFTFIDYYMSWVRQAPGKGLEWLGFIRNKANGYTTEYSASVKGRFTISRDNSKSILYLQMNSLKTEDTAVYYCTRDRPAWFVYWGQGTLVTVSS

SEQ ID NO: 135 - protein, 119 aa, Artificial Sequence
  source: /note=“Description of Artificial Sequence Synthetic polypeptide”
EVKLVESGGGLVKPGGSLRLSCAASGFTFIDYYMSWVRQAPGKGLEWLGFIRNKANGYTTEYSASVKGRFTISRDDSKNTLYLQMNSLKTEDTAVYYCTTDRPAWFVYWGQGTLVTVSS

SEQ ID NO: 136 - protein, 119 aa, Artificial Sequence
  source: /note=“Description of Artificial Sequence Synthetic polypeptide”
EVQLVESGGGLVQPGGSLRLSCAASGFTFIDYYMSWVRQAPGKGLEWVGFIRNKANGYTTEYSASVKGRFTISRDDSKNSLYLQMNSLKTEDTAVYYCARDRPAWFVYWGQGTLVTVSS

SEQ ID NO: 137 - protein, 119 aa, Artificial Sequence
  source: /note=“Description of Artificial Sequence Synthetic polypeptide”
EVQLVESGGGLVQPGGSLRLSCATSGFTFIDYYMSWVRQAPGKGLEWLGFIRNKANGYTTEYSASVKGRFTISRDNSKSILYLQMNSLKTEDTAVYYCTRDRPAWFVYWGQGTLVTVSS

SEQ ID NO: 138 - protein, 119 aa, Artificial Sequence
  source: /note=“Description of Artificial Sequence Synthetic polypeptide”
EVKLVESGGGLVQPGGSLRLSCAASGFTFIDYYMSWVRQAPGKGLEWLGFIRNKANGYTTEYSASVKGRFTISRDDSKNSLYLQMNSLKTEDTAVYYCTRDRPAWFVYWGQGTLVTVSS

SEQ ID NO: 139 - protein, 119 aa, Artificial Sequence
  source: /note=“Description of Artificial Sequence Synthetic polypeptide”
EVKLVESGGGLVQPGGSLRLSCAASGFTFIDYYMSWVRQAPGKGLEWLGFIRNKANGYTTEYSASVKGRFTISRDDSKNSLYLQMNSLKTEDTAVYYCARDRPAWFVYWGQGTLVTVSS

SEQ ID NO: 140 - protein, 113 aa, Artificial Sequence
  source: /note=“Description of Artificial Sequence Synthetic polypeptide”
DIVMTQSPDSLAVSLGERATINCKSSQSLLYSSNQKNYLAWYQQKPGQPPKLLIYWASTRESGVPDRFSGSGSGTDFTLTISSLQAEDVAVYYCQQYYSYPYTFGGGTKVEIK

SEQ ID NO: 141 - protein, 113 aa, Artificial Sequence; identical to SEQ ID NO: 88
  source: /note=“Description of Artificial Sequence Synthetic polypeptide”

SEQ ID NO: 142 - protein, 113 aa, Artificial Sequence; identical to SEQ ID NO: 107
  source: /note=“Description of Artificial Sequence Synthetic polypeptide”

SEQ ID NO: 143 - protein, 123 aa, Artificial Sequence
  source: /note=“Description of Artificial Sequence Synthetic polypeptide”
QVQLVESGGGVVQPGRSLRLSCAASGFTFSDYTMAWVRQAPGKGLEWVATIIYDGRGTYYRDSVKGRFTISRDNSKNTLYLQMNSLRAEDTAVYYCARQSDGTYYYWGYFDYWGQGTMVTVSS

SEQ ID NO: 144 - protein, 123 aa, Artificial Sequence
  source: /note=“Description of Artificial Sequence Synthetic polypeptide”
EVQLVESGGGVVQPGRSLRLSCAASGFTFSDYTMAWVRQAPGKGLEWVATIIYDGRGTYYRDSVKGRFTISRDNSKNTLYLQMNSLRAEDTAVYYCARQSDGTYYYWGYFDYWGQGTMVTVSS

SEQ ID NO: 145 - protein, 123 aa, Artificial Sequence
  source: /note=“Description of Artificial Sequence Synthetic polypeptide”
EVQLVESGGGLVQPGGSLRLSCAASGFTFSDYTMAWVRQAPGKGLEWVATIIYDGRGTYYRDSVKGRFTISRDNAKNSLYLQMNSLRAEDTAVYYCARQSDGTYYYWGYFDYWGQGTMVTVSS

SEQ ID NO: 146 - protein, 123 aa, Artificial Sequence
  source: /note=“Description of Artificial Sequence Synthetic polypeptide”
EVQLVESGGGLVQPGGSLRLSCAASGFTFSDYTMAWVRQAPGKGLEWVATIIYDGRGTYYRDSVKGRFTISRDNAKNTLYLQMNSLRAEDTAVYYCARQSDGTYYYWGYFDYWGQGTMVTVSS

SEQ ID NO: 147 - protein, 123 aa, Artificial Sequence
  source: /note=“Description of Artificial Sequence Synthetic polypeptide”
QVQLVQSGAEVKKPGASVKVSCKASGFTFSDYTMAWVRQAPGQGLEWMGTIIYDGRGTYYRDSVKGRVTMTRDTSTSTVYMELSSLRSEDTAVYYCARQSDGTYYYWGYFDYWGQGTMVTVSS

SEQ ID NO: 148 - protein, 123 aa, Artificial Sequence
  source: /note=“Description of Artificial Sequence Synthetic polypeptide”
EVQLVQSGAEVKKPGASVKVSCKASGFTFSDYTMAWVRQAPGQGLEWMGTIIYDGRGTYYRDSVKGRVTMTRDTSTSTVYMELSSLRSEDTAVYYCARQSDGTYYYWGYFDYWGQGTMVTVSS

SEQ ID NO: 149 - protein, 123 aa, Artificial Sequence
  source: /note=“Description of Artificial Sequence Synthetic polypeptide”
EVQLVQSGAEVKKPGASVKVSCKASGFTFSDYTMAWVRQAPGQGLEWVATIIYDGRGTYYRDSVKGRFTITRDNSTSTLYLELSSLRSEDTAVYYCARQSDGTYYYWGYFDYWGQGTMVTVSS

SEQ ID NO: 150 - protein, 123 aa, Artificial Sequence
  source: /note=“Description of Artificial Sequence Synthetic polypeptide”
EVQLVQSGAEVKKPGASVKVSCKASGFTFSDYTMAWVRQAPGQGLEWVGTIIYDGRGTYYRDSVKGRFTITRDNSTSTLYMELSSLRSEDTAVYYCARQSDGTYYYWGYFDYWGQGTMVTVSS

SEQ ID NO: 151 - protein, 113 aa, Artificial Sequence
  source: /note=“Description of Artificial Sequence Synthetic polypeptide”
DIVMTQSPDSLAVSLGERATINCKSSQSLLFSGNQKNYLAWYQQKPGQPPKLLIYWASTRQSGVPDRFSGSGSGTDFTLTISSLQAEDVAVYYCQQYYDSPYTFGQGTKLEIK

SEQ ID NO: 152 - protein, 113 aa, Artificial Sequence
  source: /note=“Description of Artificial Sequence Synthetic polypeptide”
DIVMTQTPLSLPVTPGEPASISCKSSQSLLFSGNQKNYLAWYLQKPGQSPQLLIYWASTRQSGVPDRFSGSGSGTDFTLKISRVEAEDVGVYYCQQYYDSPYTFGQGTKLEIK

SEQ ID NO: 153 - protein, 113 aa, Artificial Sequence
  source: /note=“Description of Artificial Sequence Synthetic polypeptide”
DIVMTQTPLSLPVTPGEPASINCKSSQSLLFSGNQKNYLAWYLQKPGQSPQLLIYWASTRQSGVPDRFSGSGSGTDFTLKISRVEAEDVGIYYCQQYYDSPYTFGQGTKLEIK

SEQ ID NO: 154 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 107 aa, Homo sapiens [sequence omitted]

SEQ ID NO: 157 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 158 - protein, 449 aa, Artificial Sequence
  source: /note=“Description of Artificial Sequence Synthetic polypeptide”
EVQLVESGGGLVQPGGSLRLSCATSGFTFIDYYMSWVRQAPGKGLEWLGFIRNKANRYTTEYSASVKGRFTISRDNSKSILYLQMNSLKTEDTAVYYCTRDRPAWFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 220 aa, Artificial Sequence
  source: /note=“Description of Artificial Sequence Synthetic polypeptide”
DIVMTQTPLSLPVTPGEPASISCKSSQSLLYSSNQKNYLAWYLQKPGQSPQLLIYWASTRESGVPDRFSGSGSGTDFTLKISRVEAEDVGVYYCQQYYSYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - protein, 449 aa, Artificial Sequence
  source: /note=“Description of Artificial Sequence Synthetic polypeptide”
EVQLVESGGGLVQPGGSLRLSCATSGFTFIDYYMSWVRQAPGKGLEWLGFIRNKAYGYTTEYSASVKGRFTISRDNSKSILYLQMNSLKTEDTAVYYCTRDRPAWFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 220 aa, Artificial Sequence; identical to SEQ ID NO: 159
  source: /note=“Description of Artificial Sequence Synthetic polypeptide”

SEQ ID NO: 162 - protein, 453 aa, Artificial Sequence
  source: /note=“Description of Artificial Sequence Synthetic polypeptide”
EVQLVQSGAEVKKPGASVKVSCKASGFTFSDYTMAWVRQAPGQGLEWVATIIYSGRGTYYRDAVKGRFTITRDNSTSTLYLELSSLRSEDTAVYYCARQSDDTYYYWGYFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 220 aa, Artificial Sequence
  source: /note=“Description of Artificial Sequence Synthetic polypeptide”
DIVMTQTPLSLPVTPGEPASINCKSSQSLLFSGNQKNYLAWYLQKPGQSPQLLIYWASTRQSGVPDRFSGSGSGTDFTLKISRVEAEDVGIYYCQQYYDVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 453 aa, Artificial Sequence
  source: /note=“Description of Artificial Sequence Synthetic polypeptide”
EVQLVQSGAEVKKPGASVKVSCKASGFTFSDYTMAWVRQAPGQGLEWVATIIYDARGTYYRDAVKGRFTITRDNSTSTLYLELSSLRSEDTAVYYCARQSDDTYYYWGYFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 220 aa, Artificial Sequence
  source: /note=“Description of Artificial Sequence Synthetic polypeptide”
DIVMTQTPLSLPVTPGEPASINCKSSQSLLFSGNQKNYLAWYLQKPGQSPQLLIYWASTRQSGVPDRFSGSGSGTDFTLKISRVEAEDVGIYYCQQYYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 166 - protein, 4 aa, Artificial Sequence
  source: /note=“Description of Artificial Sequence Synthetic peptide”
GFLG

SEQ ID NO: 167 - protein, 4 aa, Artificial Sequence
  source: /note=“Description of Artificial Sequence Synthetic peptide”
ALAL